(19) United States
(12) Patent Application Publication
(10) Pub. No.: US 2019/0134005 A1
(43) Pub. Date: May 9, 2019

(54) CERAMIDE GALACTOSYLTRANSFERASE INHIBITORS FOR THE TREATMENT OF DISEASE

(71) Applicant: BioMarin Pharmaceutical Inc., Novato, CA (US)

(72) Inventor: Bing Wang, San Jose, CA (US)

(21) Appl. No.: 16/308,414

(22) PCT Filed: Jun. 9, 2017
(86) PCT No.: PCT/US2017/036756

§ 371 Date: Dec. 7, 2018

Related U.S. Application Data
(60) Provisional application No. 62/348,826, filed on Jun. 10, 2016.
(60) Provisional application No. 62/477,962, filed on Mar. 28, 2017.

(51) Int. Cl. A61K 31/4166; A61K 31/4178

(52) U.S. Cl. CPC A61K 31/4166; A61P 25/00; A61K 31/4178

(57) ABSTRACT

Described herein are compounds, methods of making such compounds, pharmaceutical compositions and medicaments containing such compounds, and methods of using such compounds to treat or prevent diseases or disorders associated with the enzyme ceramide galactosyltransferase (CGT), such as, for example, lysosomal storage diseases. Examples of lysosomal storage diseases include, for example, Krabbe disease and Metachromatic Leukodystrophy.

### CROSS REFERENCE

This application claims the benefit of priority of U.S. Provisional Patent Application No. 62/348,826, filed Jun. 10, 2016, and U.S. Provisional Patent Application No. 62/477,962, filed Mar. 28, 2017, the content of each of which is incorporated herein by reference in its entirety.

### FIELD

Described herein are compounds, methods of making such compounds, pharmaceutical compositions and medicaments containing such compounds, and methods to treat or prevent diseases or disorders associated with the enzyme ceramide galactosyltransferase (CGT). Also described herein is that such compounds are for use in said methods for treating or preventing diseases or disorders. Such diseases or disorders include, for example, lysosomal storage diseases (LSDs). Examples of lysosomal storage diseases include Krabbe disease and Metachromatic Leukodystrophy.

### BACKGROUND

Ceramide galactosyltransferase (CGT) is a key enzyme in glycosphingolipid (GSL) biosynthesis in eukaryotic cells. Glycosphingolipids (GSLs) are believed to be integral in many cell membrane events, including cellular interactions, signaling, and trafficking. Ceramides play a central role in sphingolipid metabolism, and CGT facilitates conversion of ceramides to galactosylceramides. Galactosylceramides can be further modified by the enzyme cerebroside sulfotransferase (CST) to form sulfatides. Galactosylceramides and sulfatides are primarily produced by the myelin generating cells of the central and peripheral nervous systems, oligodendrocytes and Schwann cells respectively, where these glycolipids make up a large proportion of the lipids in the myelin sheath. Galactosylceramide and sulfatide are also found on the extracellular leaflet of the plasma membrane of other cells in eukaryotic organisms where they have been reported to be involved in a diverse range of functions.

Degradation of galactosylceramides is catalyzed in the lysosome by galactosylceramidase (GALC). Insufficient degradation of galactosylceramides, caused by deficiency of GALC, can lead to an accumulation of galactosylceramides and its partially degraded product psychosine (also called galactosylsphingosine). In humans, deficiency of GALC results in Krabbe disease (also known as globoid cell leukodystrophy or galactosylceramide lipidosis). See, e.g., Ezoe et al., J. Neurosci. Res. 59:170-178 (2000); Ezoe et al., J. Neurosci. Res. 59:179-187 (2000). Increased psychosine levels are believed to be the primary toxic molecule in Krabbe disease leading to widespread destruction of oligodendrocytes in the CNS and Schwann cells in the PNS and subsequent demyelination. See, e.g., Suzuki et al., Proc. Natl. Acad. Sci. U.S.A. 66(2):302-9 (1970); Graziano et al., Gene 555(1):2-13 (2015). Similarly, turnover of sulfatides occurs in lysosome via the enzyme arylsulfatase A (ASA) and defects in ASA can lead to the accumulation of sulfatides and its partially degraded product lyso-sulfatides. ASA deficiency can lead to the development of an autosomal recessive disease called metachromatic leukodystrophy (MLD). See, e.g., Kohlschitter, Handb. Clin. Neurol. 113:1611-1618 (2013).

Krabbe disease detrimentally affects the myelin sheath, which protects nerves and facilitates the sending and receiving of nerve signals. The accumulation of unmetabolized galactosylceramides and psychosine detrimentally affects the growth and development of the myelin sheath. Damage to the myelin sheath can lead to a severe degeneration of motor skills, cognitive deficits, and seizures, and is often fatal. Similarly, in MLD, accumulation of sulfatides and lyso-sulfatides detrimentally affects the myelin sheath, disrupting neuronal functions and leads to seizures, progressive coordination and speech problems, and other behavioral disturbances.

An approach to treatment of such diseases resulting from an abnormal accumulation of galactosylceramides, psychosine, sulfatides, lyso-sulfatides and related GSLs is to inhibit the CGT enzyme to reduce the synthesis of galactosylceramides and other downstream molecules. Accordingly, molecules that inhibit the activity of CGT are useful as therapeutic agents in the treatment of lysosomal storage diseases relating to defects in sphingolipid metabolism, such as Krabbe disease and MLD. In addition, abnormal metabolism of galactosylceramides and sulfatides has been associated with other pathological conditions such as Parkinson's Disease. See, e.g., Marshall and Bongarzone, J. Neurosci. Res., 94:1328 (2016); Smith et al., J. Pathol. 232:509 (2014). Overexpression of sulfatide has also been linked to epilepsy and audiogenic seizures as well as other pathological states in the nervous system. Accordingly, molecules that inhibit the activity of CGT may be used to treat Parkinson's disease, epilepsy and audiogenic seizures that are associate with overexpression or accumulation of galactosylceramides/sulfatides.

### SUMMARY

In one aspect, provided herein is a compound of Formula I:

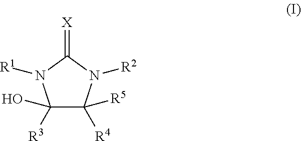

wherein:
X is O or S;
R¹ is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, cycloalkyl, heterocycloalkyl, phenyl, or thienyl; wherein alkyl is optionally substituted with halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino;
   or a single stereoisomer, mixture of stereoisomers, or pharmaceutically acceptable salt thereof.

In certain embodiments, provided herein is a compound of Formula II:

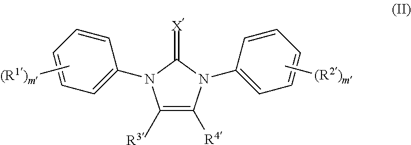

wherein:
X′ is O or S;
R^{1′} is halo, cyano, alkyl, haloalkyl, or alkoxy;
R^{2′} is halo, cyano, alkyl, haloalkyl, or alkoxy;
R^{3′} is alkyl, phenyl, heteroaryl with 5-6 ring atoms, or phenylcarbonyl, wherein the phenyl, heteroaryl, or phenylcarbonyl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl;
R^{4′} is hydrogen, alkyl, alkoxyalkyl, C₃₋₅ cycloalkyl, or 3-6 membered heterocycloalkyl, wherein the cycloalkyl and heterocycloalkyl groups are each independently optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R^{3′} and R^{4′} and the carbons to which they are attached combine to form a 5-6 membered cycloalkylene;
m′ is 1 or 2;
n′ is 1 or 2;
with the proviso that when R^{1′} and R^{2′} are each methoxy, then R^{3′} cannot be methoxy-substituted phenyl;
   or a pharmaceutically acceptable salt thereof.

In another aspect, provided herein is a compound of Formula VII:

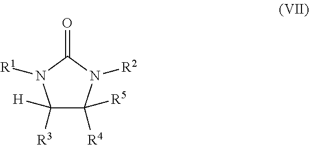

wherein:
R¹ is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is aryl optionally substituted with a group selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, cycloalkyl, or aryl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 groups selected from halo, cyano, hydroxy, C₁₋₃ alkyl, alkoxy, amino, alkylamino, and dialkylamino; and
provided that R⁴ and R⁵ are not both hydrogen; and
provided that the compound is not (4S,5S)-4-(tert-butyl)-1,3-bis(4-methoxyphenyl)-5-phenylimidazolidin-2-one; and
   optionally a single stereoisomer or mixture of stereoisomers thereof and additionally optionally a pharmaceutically acceptable salt thereof.

In certain embodiments, provided herein is a pharmaceutical composition comprising a compound disclosed herein. Such a compound is, for example, a compound of Formula I or II, or a compound of Formula VII, optionally as a single stereoisomer, mixture of stereoisomers, and additionally optionally as a pharmaceutically acceptable salt thereof, and a pharmaceutically acceptable excipient.

In another aspect, provided herein is a method of treating a lysosomal storage disease with a compound disclosed herein. Thus, a compound disclosed herein is for use in a method of treating a lysosomal storage disease. Such a compound is, for example, a compound of Formula I or II, or a compound of Formula VII, optionally as a single stereoisomer, mixture of stereoisomers, and additionally optionally as a pharmaceutically acceptable salt thereof, or the pharmaceutical composition thereof additionally comprising a pharmaceutically acceptable excipient.

In certain embodiments, the lysosomal storage disease is Krabbe disease or Metachromatic Leukodystrophy.

### DETAILED DESCRIPTION

Abbreviations

| Abbreviation | Meaning |
|---|---|
| ASA | arylsulfatase A |
| BAST | bis(2-methoxyethyl)aminosulfur trifluoride |
| BF₃•Et₂O | boron trifluoride diethyl etherate |
| Boc₂O | di-tert-butyl dicarbonate |
| CbzCl | benzyl chloroformate |
| CDCl₃ | deuterated chloroform |
| CHAPS | 3-[(3-cholamidopropyl)dimethylammonio]-1- |
| | propanesulfonate |
| CH₃CN | methylcyanide |
| CGT | ceramide galactosyltransferase |
| m-CPBA | meta-chloroperoxybenzoic acid |
| DCM | dichloromethane |
| DEA | diethylamine |
| DEAD | diethyl azodicarboxylate |
| DIAD | diisopropyl azodicarboxylate |
| DIPEA | diisoproylethylamine |
| DMF | dimethylformamide |
| DMSO | dimethyl sulfoxide |
| DMP | Dess-Martin periodinane |
| EDCI | 1-ethyl-3-(3-dimethylaminopropyl)carbodiimide |
| EGTA | ethylene glycol-bis(β-aminoethyl ether)-N,N,N′,N′- |
| | tetraacetic acid |
| ESI | electrospray ionization |
| EtOH | ethanol |
| Et₂O | diethyl ether |
| Et₃N | triethylamine |
| GALC | galactosylceramidase |
| GSL | glycosphingolipids |
| H₂SO₄ | sulfuric acid |
| HATU | O-(7-azabenzotriazol-1-yl)-N,N,N′,N′- |
| | tetramethyluronium hexafluorophosphate |
| HPLC | high performance liquid chromatography |
| Hz | Hertz (s⁻¹) |
| KCl | potassium chloride |
| KOH | potassium hydroxide |
| LC-MS | liquid chromatography-mass spetrometry |
| LDA | lithium diisopropylamide |
| mg | milligram |
| MeOH | methanol |
| M-PER | Mammalian Protein Extraction Reagent |
| MHz | MegaHertz |
| mL | milliliter |
| mm | millimeter |
| mmol | millimole |
| MLD | metachromatic leukodystrophy |
| MsCl | methanesulfonyl chloride (mesyl chloride) |
| μL | microliter |
| mM | millimolar |
| μM | micromole |
| n-BuLi | n-butyllithium |
| NaOAc | sodium acetate |
| Na₂SO₄ | sodium sulfate |
| NBS | N-bromosuccinimide |
| NMP | N-methyl pyrrolidone |
| NMR | Nuclear Magnetic Resonance |
| ppm | parts per million |
| PdCl₂(PPh₃)₂ | bis(triphenylphosphine)palladium(II) dichloride |
| POCl₃ | phosphoryl chloride |
| PPTS | pyridinium p-toluenesulfonate |
| TBAF | tetra-n-butylammonium fluoride |
| TEA | triethylamine |
| TFA | trifluoroacetic acid |
| THF | tetrahydrofuran |
| TMS | trimethylsilane |
| TMSCHN₂ | trimethylsilyldiazomethane |
| TMSI | trimethylsilyl |
| UDP- | uridine diphosphate galactose |
| galactose | |

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art.

“About” preceding a numerical value refers to a range of values ±10% of the value specified.

“Acceptable” with respect to a formulation, composition or ingredient, as used herein, means having no persistent detrimental effect on the general health of the subject being treated.

Whenever a group is described as being “optionally substituted,” it is meant that the referenced group can be “unsubstituted or substituted.”

“Alkenyl” means a straight or branched hydrocarbon radical having from 2 to 8 carbon atoms and at least one double bond and in certain embodiments include ethenyl, propenyl, 1-but-3-enyl, 1-pent-3-enyl, or 1-hex-5-enyl. As used herein, the terms “radical” and “group” are used interchangeably and are understood to have the same meaning.

“Alkoxy” means a group of the formula —OR, where R is alkyl. In certain embodiments, alkoxy includes methoxy, ethoxy, propoxy, 2-propoxy, butoxy, tert-butoxy, pentyloxy, or hexyloxy.

“Alkylcarbonylamino” means a group of the formula —NHC(O)R, where R is alkyl, as defined herein.

“Alkoxyalkyl” means a group of the formula —R—O—R′, where R and R′ are independently alkyl as defined herein.

“Alkoxyalkylene” means a group of the formula —R—O—R′, where R is alkylene and R′ is alkyl as defined herein.

“Alkoxycarbonyl” means a group of the formula —C(O)R, where R is alkoxy, as defined herein.

“Alkyl” means a straight or branched saturated hydrocarbon radical containing from 1-10 carbon atoms, and in certain embodiments includes 1-6 carbon atoms.

In certain embodiments, alkyl includes 1-4 carbon atoms, and in certain embodiments includes 1-3 carbon atoms.

When an alkyl group contains from 1-10 carbon atoms, it may be referred to herein as C₁₋₁₀ alkyl. When an alkyl group contains from 1-6 carbon atoms, it may be referred to herein as C₁₋₆ alkyl. When an alkyl group contains from 1-4 carbon atoms, it may be referred to herein as C₁₋₄ alkyl. When an alkyl group contains from 1-3 carbon atoms, it may be referred to herein as C₁₋₃ alkyl.

In certain embodiments, alkyl includes methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

“Alkylamino” means a group of the formula —NHR, where R is alkyl as defined herein, or an N-oxide derivative thereof, e.g., methylamino, ethylamino, n-, iso-propylamino, n-, iso-, tert-butylamino, or methylamino-N-oxide, and the like.

“Alkylaminocarbonyl” means a group of the formula —C(O)R, where R is alkylamino, as defined herein.

“Alkylcarbonyl” means a group of the formula —C(O)R, where R is alkyl, as defined herein.

“Alkylene” means a divalent radical formed by removal of a hydrogen atom from alkyl.

“Alkynyl” means a straight or branched hydrocarbon radical having from 2 to 8 carbon atoms and at least one triple bond and includes ethynyl, propynyl, 1-but-3-ynyl, 1-pent-3-ynyl, 1-hex-5-ynyl and the like.

“Amino” means an —NH₂ group.

“Aminocarbonyl” means an —C(O)NH₂ group.

“Aryl” means a monovalent six- to fourteen-membered, mono-, bi-, or tri-carbocyclic ring, wherein the monocyclic ring is aromatic and at least one of the rings in the bicyclic or tricyclic ring is aromatic. In certain embodiments, aryl includes phenyl, naphthyl, indanyl, or anthracenyl.

“Carbonyl” means an —C═(O) group.

“Carboxy” means an —C(O)OH group.

“Cyano” means an —CN group.

“Cyanoalkyl” means and alkyl group substituted with a cyano group, as defined herein.

“Cycloalkyl” means a monocyclic or bicyclic, saturated or partially unsaturated (but not aromatic), hydrocarbon radical of three to ten carbon ring atoms.

In certain embodiments, one or two ring carbon atoms of the cycloalkyl group atoms may be replaced by a C(O)—, —C(S)—, or C(═NH)— group.

When a cycloalkyl group contains from 3-10 carbon atoms, it may be referred to herein as C₃₋₁₀ cycloalkyl. When a cycloalkyl group contains from 5-6 carbon atoms, it may be referred to herein as C₅₋₆ cycloalkyl.

Cycloalkyl groups include fused, bridged and spirocycloalkyl bicyclic rings. For example, when fused, the cycloalkyl group may comprise two rings that share adjacent atoms (e.g., one covalent bond). When bridged, the cycloalkyl group may comprise two rings that share three or more atoms, separating the two bridgehead atoms by a bridge containing at least one atom. When spiro, the cycloalkyl group may comprise two rings that share only one single atom, the spiro atom, which may be, for example, a quaternary carbon.

In certain embodiments, cycloalkyl groups include cyclopropyl, cyclobutyl, cyclopentyl, or cyclohexyl.

In certain embodiments, cycloalkyl groups include:

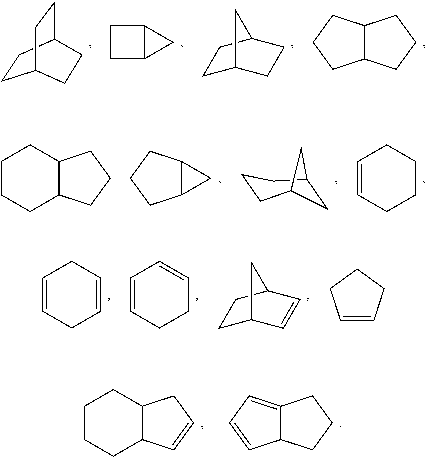

“Cycloalkylene” means a divalent radical formed by removal of a hydrogen atom from cycloalkyl. When a cycloalkylene group contains from 5-6 carbon atoms, it may be referred to herein as C₅₋₆ cycloalkylene.

“(Cycloalkyl)alkyl” means an alkyl group, as defined herein, substituted with at least one, in another example 1 or 2, cycloalkyl groups as defined herein.

“(Cycloalkyl)alkoxy” means a group of the formula —OR, where R is a (cycloalkyl)alkyl group as defined herein.

“Cycloalkyloxy” means a group of the formula —OR, where R is cycloalkyl, as defined herein.

“Dialkylamino” means a group of the formula —NRR′, where R and R′ are independently alkyl as defined herein, or an N-oxide derivative, or a protected derivative thereof, e.g., dimethylamino, diethylamino, N,N-methylpropylamino or N,N-methylethylamino, and the like.

“Dialkylaminocarbonyl” means a group of the formula —C(O)R, where R is dialkylamino, as defined herein.

“Halo” means a fluoro, chloro, bromo, or iodo group.

“Haloalkoxy” means an alkoxy group, substituted with one or more halo atoms, and in some embodiments by 1, 2, or 3 halo atoms.

Certain embodiments of haloalkoxy include difluoromethoxy, trifluoromethoxy, or 1,1,1-trifluoroethoxy.

“Haloalkyl” means an alkyl group substituted with one or more halo atoms, and in certain embodiments by 1, 2, 3, 4, 5, or 6 halo atoms, and in certain embodiments by 1, 2, or 3 halo atoms. Examples include, but are not limited to, trifluoromethyl, chloromethyl, and the like.

In certain other embodiments, haloalkyl is substituted with 2 halo atoms, and in certain embodiments by 1 halo atom.

Certain other embodiments of haloalkyl include chloromethyl, fluoromethyl, difluoromethyl, trifluoromethyl, or 1,1,1-trifluoroethanyl.

“Heteroaryl” means monocyclic, fused bicyclic, or fused tricyclic, radical of 5 to 14 ring atoms containing one or more, in another example one, two, three, or four ring heteroatoms independently selected from —O—, —S(O)ₙ— (n is 0, 1, or 2), —N=(trivalent nitrogen), —N(H)—, and N-oxide, and the remaining ring atoms being carbon, wherein the ring comprising a monocyclic radical is aromatic and wherein at least one of the fused rings comprising a bicyclic or tricyclic radical is aromatic (but does not have to be a ring which contains a heteroatom, e.g. 2,3-dihydrobenzo[b][1,4]dioxin-6-yl). One or two ring carbon atoms of any nonaromatic rings comprising a bicyclic or tricyclic radical may be replaced by a —C(O)—, —C(S)—, or —C(═NH)— group. Fused bicyclic radical includes bridged ring systems. Unless stated otherwise, the valency may be located on any atom of any ring of the heteroaryl group, valency rules permitting.

In certain embodiments, heteroaryl includes, but is not limited to, triazolyl, tetrazolyl, pyrrolyl, imidazolyl, thienyl, furanyl, pyrazolyl, oxazolyl, isooxazolyl, oxadiazolyl, thiadiazolyl, indolyl, 2,3-dihydro-1H-indolyl (including, for example, 2,3-dihydro-1H-indol-2-yl or 2,3-dihydro-1H-indol-5-yl, and the like), indazolyl, phthalimidyl, benzimidazolyl, benzoxazolyl, benzofuranyl, benzothienyl, benzopyranyl, benzothiazolyl, pyridinyl, pyrazinyl, pyrimidinyl, pyridazinyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrrolo[1,2-b]pyridazinyl, imidazo[1,2-a]pyridinyl, thiazolyl, benzo[d][1,3]dioxolyl, 2,3-dihydrobenzo[b][1,4]dioxinyl, furo[2,3-d]thiazolyl, thieno[2,3-d]oxazolyl, thieno[3,2-b]furanyl, furo[2,3-d]pyrimidinyl, furo[3,2-b]pyridinyl, furo[3,2-c]pyridinyl, 6,7-dihydro-5H-cyclopenta[b]pyridinyl, and 7,8-dihydro-6H-cyclopenta[g]quinoxalinyl; and N-oxide and protected derivatives thereof.

“Heterocycloalkyl” means a saturated or partially unsaturated (but not aromatic) monovalent monocyclic group of 3 to 9 ring atoms or a saturated or partially unsaturated (but not aromatic) monovalent or bicyclic group of 5 to 12 ring atoms in which one or more ring atoms is a heteroatom independently selected from —O—, —S(O)ₙ— (n is 0, 1, or 2), —N=(trivalent nitrogen), or —NH—, and the remaining ring atoms are carbon. In certain embodiments, the heterocycloalkyl group comprises one, two, three, or four ring heteroatoms, independently selected from —O—, —S(O)ₙ— (n is 0, 1, or 2), —N=(trivalent nitrogen), or —NH—.

In certain embodiments, the heterocycloalkyl group comprises two, three, or four ring heteroatoms, independently selected from —O—, —S(O)ₙ— (n is 0, 1, or 2), —N═, —NH—, and N-oxide, the remaining ring atoms being carbon. One or two ring carbon atoms may be replaced by a C(O)—, —C(S)—, or C(═NH)— group.

When a heterocycloalkyl group contains from 5 to 12 ring atoms, it may be referred to herein as 5-12 membered heterocycloalkyl. When a heterocycloalkyl group contains from 5 to 6 ring atoms, it may be referred to herein as 5-6 membered heterocycloalkyl.

Heterocycloalkyl groups include fused, bridged and spiro heterocycloalkyl bicyclic rings. For example, when fused, the heterocycloalkyl group may comprise two rings that share adjacent atoms (e.g., one covalent bond). When bridged, the heterocycloalkyl group may comprise two rings that share three or more atoms, separating the two bridgehead atoms by a bridge containing at least one atom. When spiro, the heterocycloalkyl group may comprise two rings that share only one single atom, the spiro atom, which may be, for example, a quaternary carbon.

In certain embodiments, heterocycloalkyl includes, but is not limited to, azetidinyl, pyrrolidinyl, 2-oxopyrrolidinyl, 2,5-dihydro-1H-pyrrolinyl, 2,5-dioxo-1H-pyrrolyl, 2,5-dioxo-pyrrolidinyl, 2,5-dihydro-1H-pyrrolyl, piperidinyl, 2-oxopiperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, 2-azaspiro[3.3]heptanyl, 7-azabicyclo[2.2.1]heptanyl, and 8-azabicyclo[3.2.1]octanyl, and the N-oxide (for example 1-oxido-pyrrolidin-1-yl) or a protected derivative thereof.

In certain embodiments, heterocycloalkyl includes, but is not limited to, azetidinyl, pyrrolidinyl, 2-oxopyrrolidinyl, 2,5-dihydro-1H-pyrrolinyl, 2,5-dioxo-1H-pyrrolyl, 2,5-dioxo-pyrrolidinyl, 2,5-dihydro-1H-pyrrolyl, piperidinyl, 2-oxopiperidinyl, 4-piperidonyl, morpholinyl, piperazinyl, 2-oxopiperazinyl, dioxopiperazinyl, pyranyl, tetrahydropyranyl, tetrahydrothiopyranyl, 1,3-dioxinyl, 1,3-dioxanyl, 1,4-dioxinyl, 1,4-dioxanyl, thiomorpholinyl, thiamorpholinyl, perhydroazepinyl, pyrazolidinyl, imidazolinyl, imidazolidinyl, 2,4-dioxo-imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, octahydropyrrolo[3,4-c]pyrrolinyl, decahydroisoquinolyl, tetrahydrofuryl, 2-azaspiro[3.3]heptanyl, 4,7-diazaspiro[2.5]octane, 1,6-diazaspiro[3.3]heptanyl, 7-azabicyclo[2.2.1]heptanyl, and 8-azabicyclo[3.2.1]octanyl.

“Hydroxyalkyl” means an alkyl group, as defined herein, substituted with at least one, or in other embodiments 1, 2, or 3 hydroxy groups.

“Hydroxyalkoxy” means an alkoxy group, as defined herein, substituted with at least one, or in other embodiments 1, 2, or 3 hydroxy groups.

“Hydroxy” means an —OH group.

The terms “hydroxy” and “hydroxyl” are used interchangeably and mean an —OH group.

“Nitro” means an —NO₂ group.

“Phenylcarbonyl” means an —C(O)-phenyl group.

“Spirocycloalkyl” means alkylene, where both ends of which are attached to the same carbon atom to form a ring.

In certain embodiments, spirocycloalkyl includes C₃-spirocycloalkyl (i.e., spirocyclopropyl), C₄-spirocycloalkyl, C₅-spirocycloalkyl, C₆-spirocycloalkyl, C₇-spirocycloalkyl, or C₅-spirocycloalkyl. Certain embodiments of spirocycloalkyl include spirocyclobutyl or spirocyclopentyl.

“Spiroheterocycloalkyl” means spirocycloalkyl, as defined herein, having one or two CH₂ moieties replaced with independently selected O, C(O), S, S(O), SO₂ or NH and one or two CH moieties unreplaced or replaced with N.

Certain embodiments of spiroheterocycloalkyl include

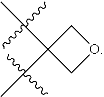 [SMILES: *C1(*)COC1]

Certain embodiments of spiroheterocycloalkyl include

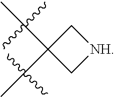 [SMILES: *C1(*)CNC1]

In some embodiments, compounds of the described herein exist as stereoisomers, wherein asymmetric or chiral centers are present. Stereoisomers are designated (R) or (S) depending on the configuration of substituents around the chiral carbon atom. The term (R) and (S) used herein are configurations as defined in IUPAC 1974 Recommendations for Section E, Fundamental Stereochemistry, Pure Appl. Chem., (1976), 45:13-30, hereby incorporated by reference. The embodiments described herein specifically includes the various stereoisomers and mixtures thereof.

“Stereoisomers” include (but are not limited to) geometric isomers, enantiomers, diastereomers, and mixtures of geometric isomers, enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic column.

“Amelioration” of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The terms “effective amount” or “therapeutically effective amount,” refer to a sufficient amount of an agent or a compound being administered which will relieve to some extent one or more of the symptoms of the disease or disorder being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

“Excipient” or “pharmaceutically acceptable excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

“Pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts are not specifically limited as far as it can be used in medicaments. Examples of a salt that the compound forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components, such as an excipient. The pharmaceutical composition facilitates administration of the compound to an organism. Multiple techniques of administering a compound exist in the art including, but not limited to: intravenous, oral, aerosol, parenteral, ophthalmic, pulmonary and topical administration.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

In certain embodiments, the subject is a mammal. In certain embodiments, the subject is a human. In certain embodiments, the subject is an adult human. In certain embodiments, the subject is a human child.

“Treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

### EMBODIMENTS

The following paragraphs present a number of embodiments of the compounds disclosed herein. In each instance the embodiment includes both the recited compound(s) as well as a single stereoisomer or mixture of stereoisomers thereof, as well as a pharmaceutically acceptable salt thereof.

### Compounds of Formulas I and II

In one aspect, provided is a compound of Formula I:

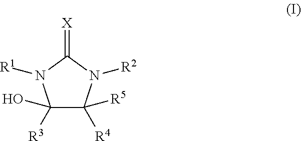

wherein:
X is O or S;
R¹ is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino;
   or a single stereoisomer, mixture of stereoisomers, or pharmaceutically acceptable salt thereof.

In certain embodiments, provided is a compound of Formula I:

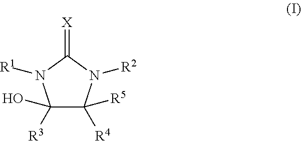

wherein:
X is O or S;
R¹ is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, haloalkyl, cycloalkyl, heterocycloalkyl, phenyl, or thienyl; wherein the alkyl is optionally substituted with 1 or 2 groups selected from cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino;
   or a single stereoisomer, mixture of stereoisomers, or pharmaceutically acceptable salt thereof.

In certain embodiments, provided is a compound of Formula I:

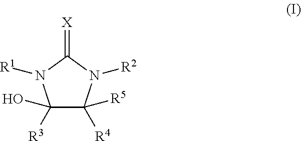

wherein:
X is O or S;
R¹ is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, haloalkyl, cycloalkyl, heterocycloalkyl, phenyl, or thienyl; wherein the alkyl is optionally substituted with 1 or 2 groups selected from cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino;
provided that the compound of formula I is not:

| No. | Structure | Name |
|---|---|---|
| 1. | 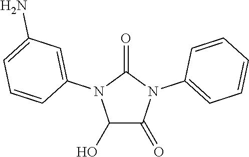 [SMILES: Nc1cccc(N2C(=O)N(c3ccccc3)C(=O)C2O)c1] | 1-(3-aminophenyl)-5- hydroxy-3- phenylimidazolidine- 2,4-dione; or |
| 2. | 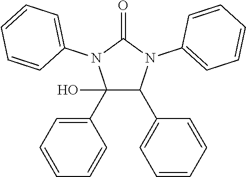 [SMILES: O=C1N(c2ccccc2)C(c2ccccc2)C(O)(c2ccccc2)N1c1ccccc1] | 4-hydroxy-1,3,4,5- tetraphenylimidazolidin- 2-one; or |

provided that the compound of Formula I is that wherein when R¹ and R² are both unsubstituted phenyl and R⁴ and R⁵ and the carbon to which they are attached combine to form carbonyl, then R³ is not methyl, ethyl, tert-butyl, trifluoromethyl, pentafluoroethyl, unsubstituted phenyl, unsubstituted naphthyl, or unsubstituted anthracenyl;
or a single stereoisomer, mixture of stereoisomers, or pharmaceutically acceptable salt thereof.

In certain embodiments, the compound of Formula I is that wherein:
X is O or S;
R¹ is aryl optionally substituted with 1 or 2 groups selected from halo, cyano, alkyl, haloalkyl, alkoxy, cycloalkyloxy, amino, alkylamino, and dialkylamino;
R² is aryl optionally substituted with 1 or 2 groups selected from halo, cyano, alkyl, haloalkyl, alkoxy, cycloalkyloxy, amino, alkylamino, and dialkylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, hydroxy, alkyl, alkenyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo;
R⁴ is hydrogen, alkyl, cycloalkyl, heterocycloalkyl, phenyl, or thienyl; wherein the alkyl is optionally substituted with 1 or 2 groups selected from halo, alkoxy, amino, alkylamino, dialkylamino; and
R⁵ is hydrogen, alkyl, or cycloalkyl;
or R⁴ and R⁵ and the carbon to which they are attached combine to form carbonyl, spirocycloalkyl, or spiroheterocycloalkyl optionally substituted with halo;
   or a single stereoisomer, mixture of stereoisomers, or pharmaceutically acceptable salt thereof.

In certain embodiments, the compound of Formula I is that wherein:
X is O or S;
R¹ is aryl optionally substituted with 1 or 2 groups selected from halo, cyano, alkyl, haloalkyl, alkoxy, cycloalkyloxy, amino, alkylamino, and dialkylamino;
R² is aryl optionally substituted with 1 or 2 groups selected from halo, cyano, alkyl, haloalkyl, alkoxy, cycloalkyloxy, amino, alkylamino, and dialkylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, hydroxy, alkyl, alkenyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo;
R⁴ is hydrogen, alkyl, haloalkyl, cycloalkyl, heterocycloalkyl, phenyl, or thienyl; wherein the alkyl is optionally substituted with 1 or 2 groups selected from alkoxy, amino, alkylamino, dialkylamino; and
R⁵ is hydrogen, alkyl, or cycloalkyl;
or R⁴ and R⁵ and the carbon to which they are attached combine to form carbonyl, spirocycloalkyl, or spiroheterocycloalkyl optionally substituted with halo; or a single stereoisomer, mixture of stereoisomers, or pharmaceutically acceptable salt thereof.

In certain embodiments, the compound of Formula I is that wherein: X is O; R¹ and R² are each independently phenyl optionally substituted with halo, cyano, alkyl, alkoxy, or haloalkoxy; R³ is alkyl, phenyl, or heteroaryl, wherein the phenyl is optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, alkyl, cycloalkyl, heterocycloalkyl, phenyl, or thienyl; R⁵ is hydrogen or alkyl; or R⁴ and R⁵ and the carbon to which they are attached combine to form spirocycloalkyl.

In certain embodiments, the compound of Formula I is that wherein: X is O; R¹ and R² are each independently phenyl optionally substituted with halo, cyano, alkyl, alkoxy, or haloalkoxy; R³ is alkyl, phenyl, or heteroaryl, wherein the phenyl is optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, alkyl, haloalkyl, cycloalkyl, heterocycloalkyl, phenyl, or thienyl; R⁵ is hydrogen or alkyl; or R⁴ and R⁵ and the carbon to which they are attached combine to form spirocycloalkyl.

In certain embodiments, the compound of Formula I is that wherein: X is O; R¹ and R² are each independently phenyl optionally substituted with halo, cyano, alkyl, alkoxy, or haloalkoxy; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, alkyl, or phenyl; R⁵ is hydrogen or alkyl; or R⁴ and R⁵ and the carbon to which they are attached combine to form spirocycloalkyl. In certain embodiments, X is O; R¹ and R² are each independently selected from phenyl optionally substituted with halo, cyano, alkyl, alkoxy, or haloalkoxy; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, alkyl, or phenyl; R⁵ is hydrogen or alkyl; or R⁴ and R⁵ and the carbon to which they are attached combine to form spirocycloalkyl.

In certain embodiments, the compound of Formula I is that wherein: X is O; R¹ and R² are each independently selected from phenyl optionally substituted with halo, cyano, alkyl, alkoxy, or haloalkoxy; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, methyl, ethyl or propyl; and R⁵ is hydrogen or methyl. In certain embodiments, X is O; R¹ and R² are each independently selected from phenyl optionally substituted with chloro or bromo; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, methyl, ethyl or propyl; and R⁵ is hydrogen or methyl.

In certain embodiments, the compound of Formula I is that wherein: X is O; R¹ and R² are each independently selected from phenyl optionally substituted with halo, cyano, alkyl, alkoxy, or haloalkoxy; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen or trifluoromethyl; and R⁵ is hydrogen or methyl. In certain embodiments, X is O; R¹ and R² are each independently selected from phenyl optionally substituted with chloro or bromo; R³ is phenyl optionally substituted with halo, cyano, hydroxy, alkyl, alkenyl, alkynyl, haloalkyl, hydroxyalkyl, alkoxy, haloalkoxy, alkylcarbonyl, or cycloalkyl; R⁴ is hydrogen, trifluoromethyl; and R⁵ is hydrogen or methyl. In certain embodiments, X is O; R¹ and R² are each independently selected from phenyl optionally substituted with chloro or bromo; R³ is phenyl optionally substituted with 2 groups independently selected from halo, cyano, hydroxy, alkyl, alkenyl, alkynyl, haloalkyl, hydroxyalkyl, alkoxy, haloalkoxy, alkylcarbonyl, and cycloalkyl; R⁴ is hydrogen or trifluoromethyl; and R⁵ is hydrogen or methyl.

In certain embodiments, the compound of Formula I is that wherein: X is O; R³ is hydrogen, R⁴ is alkyl or cycloalkyl, and R⁵ is optionally substituted phenyl. In certain embodiments, X is O; R³ is hydrogen, R⁴ is alkyl, and R⁵ is optionally substituted phenyl. In certain embodiments, X is O; R³ is hydrogen, R⁴ is methyl, and R⁵ is optionally substituted phenyl. In certain embodiments, X is O; R³ is hydrogen, R⁴ is methyl, and R⁵ is phenyl optionally substituted with halo, alkyl, haloalkyl, or alkoxy.

In certain embodiments, the compound of Formula I is that wherein: X is O; R³ is hydrogen, R¹ and R² are each independently selected from phenyl substituted with chloro or bromo, R⁴ is alkyl or cycloalkyl, and R⁵ is optionally substituted phenyl. In certain embodiments, X is O; R³ is hydrogen, R¹ and R² are each independently selected from phenyl substituted with chloro or bromo, R⁴ is alkyl, and R⁵ is optionally substituted phenyl. In certain embodiments, X is O; R³ is hydrogen, R¹ and R² are each independently selected from phenyl substituted with chloro or bromo, R⁴ is methyl, and R⁵ is optionally substituted phenyl. In certain embodiments, X is O; R³ is hydrogen, R¹ and R² are each independently selected from phenyl substituted with chloro or bromo, R⁴ is methyl, and R⁵ is phenyl optionally substituted with halo, alkyl, haloalkyl, or alkoxy.

In certain embodiments, the compound of Formula I is that wherein: X is O; R⁴ and R⁵ and the carbon to which they are attached form a carbonyl, and R³ is alkyl, haloalkyl, heterocycloalkyl, aryl, or heteroaryl, wherein the aryl and heteroaryl are each optionally substituted. In certain embodiments, X is O; R⁴ and R⁵ and the carbon to which they are attached form a carbonyl, and R³ is alkyl, haloalkyl, heterocycloalkyl, phenyl, or heteroaryl, wherein the aryl and heteroaryl are each optionally substituted.

In certain embodiments, the compound of Formula I is that wherein: X is O; R⁴ and R⁵ and the carbon to which they are attached form a carbonyl, and R³ is optionally substituted phenyl. In certain embodiments, X is O; R⁴ and R⁵ and the carbon to which they are attached form a carbonyl, and R³ is phenyl optionally substituted with 1 or 2 substituents selected from halo, alkyl, alkoxy, alkoxycarbonyl, (cycloalkyl)alkyl, cycloalkyloxy, (cycloalkyl)alkoxy, phenyl, halo-substituted and phenyl. In certain embodiments, X is O; R⁴ and R⁵ and the carbon to which they are attached form a carbonyl, and R³ is phenyl optionally substituted with halo, alkyl, alkoxy, alkoxycarbonyl, (cycloalkyl)alkyl, cycloalkyloxy, (cycloalkyl)alkoxy, phenyl, or halo-substituted phenyl.

In certain embodiments, the compound of Formula I is that wherein: X is O; R⁴ and R⁵ and the carbon to which they are attached form a carbonyl, and R¹ is optionally substituted phenyl. In certain embodiments, X is O; R⁴ and R⁵ and the carbon to which they are attached form a carbonyl, and R¹ is phenyl optionally substituted with 1 or 2 substituents selected from halo, alkyl, alkoxy, cycloalkyloxy, amino, alkylamino, dialkylamino, and alkoxycarbonylamino.

In certain embodiments, the compound of Formula I is that wherein: X is O; R⁴ and R⁵ and the carbon to which they are attached form a carbonyl; R¹ is phenyl optionally substituted with halo, alkyl, alkoxy, or cycloalkyloxy; and R³ is phenyl optionally substituted with halo, alkyl, alkoxy, alkoxycarbonyl, (cycloalkyl)alkyl, cycloalkyloxy, (cycloalkyl)alkoxy, phenyl, or halo-substituted phenyl.

In certain embodiments, the compound of Formula I is that wherein R¹, R² and R³ are each optionally substituted phenyl. In certain embodiments, R¹ and R² are each optionally substituted phenyl, and R³ is optionally substituted naphthyl.

In certain embodiments, the compound of Formula I is that wherein R¹ and R² are each optionally substituted phenyl, and R³ is optionally substituted pyridinyl (e.g., pyridin-2-yl, pyridin-3-yl, and pyridin-4-yl). In certain embodiments, R¹ and R² are each optionally substituted phenyl, and R³ is optionally substituted pyrrolyl. In certain embodiments, R¹ and R² are each optionally substituted phenyl, and R³ is optionally substituted furanyl. In certain embodiments, R¹ and R² are each optionally substituted phenyl, and R³ is optionally substituted thienyl (e.g., thiophen-2-yl and thiophen-3-yl). In certain embodiments, R¹ and R² are each optionally substituted phenyl, and R³ is optionally substituted thiazolyl. In certain embodiments, R¹ and R² are each optionally substituted phenyl, and R³ is optionally substituted oxazolyl. In certain embodiments, R¹ and R² are each optionally substituted phenyl, and R³ is optionally substituted indolyl (e.g., 1H-indol-2-yl). In certain embodiments, R¹ and R² are each optionally substituted phenyl, and R³ is optionally substituted benzofuranyl (e.g., benzofuran-2-yl). In certain embodiments, R¹ and R² are each optionally substituted phenyl, and R³ is optionally substituted benzothienyl (e.g., benzo[b]thiophen-2-yl). In certain embodiments, R¹ and R² are each optionally substituted phenyl, and R³ is optionally substituted benzothiazolyl (e.g., 1H-benzothiazol-2-yl). In certain embodiments, R¹ and R² are each optionally substituted phenyl, and R³ is optionally substituted benzimidazolyl (e.g., 1H-benzimidazol-2-yl).

In certain embodiments, the compound of Formula I is that wherein R¹ and R² are each optionally substituted phenyl, and R³ is optionally substituted pyrrolidinyl.

In certain embodiments, the compound of Formula I is that wherein R¹, R² and R⁴ are each optionally substituted phenyl.

In certain embodiments, the compound of Formula I is that wherein R¹ and R² are each optionally substituted phenyl, and R⁴ is optionally substituted thienyl.

In certain embodiments, the compound of Formula I is that wherein R¹ and R² are each optionally substituted phenyl, and R⁴ is optionally substituted cyclohexyl. In certain embodiments, R¹ and R² are each optionally substituted phenyl, and R⁴ is optionally substituted cyclopentyl. In certain embodiments, R¹ and R² are each optionally substituted phenyl, and R⁴ is optionally substituted cyclobutyl. In certain embodiments, R¹ and R² are each optionally substituted phenyl, and R⁴ is optionally substituted cyclopropyl.

In certain embodiments, the compound of Formula I is that where X is S.

In certain embodiments, the compound of Formula I is that where X is O.

In certain embodiments, the compound of Formula I is that where R¹ is optionally substituted phenyl.

In certain embodiments, the compound of Formula I is that where R¹ is optionally substituted with 1 or 2 groups selected from halo, cyano, alkyl, haloalkyl, alkoxy, cycloalkyloxy, amino, alkylamino, and dialkylamino.

In certain embodiments, the compound of Formula I is that where R¹ is phenyl substituted with 1 or 2 groups selected from halo, cyano, alkyl, haloalkyl, alkoxy, and cycloalkyloxy. In certain embodiments, R¹ is phenyl substituted with 1 or 2 groups selected from halo, cyano, alkyl, haloalkyl, and alkoxy. In certain embodiments, R¹ is phenyl substituted with 1 or 2 groups selected from halo and alkyl. In certain embodiments, R¹ is phenyl substituted with 1 or 2 groups selected from chloro, bromo, methyl, and ethyl. In certain embodiments, R¹ is phenyl substituted with 1 or 2 groups selected from chloro and methyl. In certain embodiments, R¹ is phenyl substituted with chloro, bromo, methyl, or ethyl. In certain embodiments, R¹ is phenyl substituted with chloro or methyl. In certain embodiments, R¹ is phenyl substituted with an alkyl group. In certain embodiments, R¹ is phenyl substituted with methyl or ethyl. In certain embodiments, R¹ is phenyl substituted with halo. In certain embodiments, R¹ is phenyl substituted with chloro, bromo, or fluoro. In certain embodiments, R¹ is phenyl substituted with fluoro. In certain embodiments, R¹ is phenyl substituted with chloro or bromo. In certain embodiments, R¹ is phenyl substituted with chloro. In certain embodiments, R¹ is phenyl substituted with bromo.

In certain embodiments, the compound of Formula I is that where R² is optionally substituted phenyl.

In certain embodiments, the compound of Formula I is that where R² is optionally substituted with 1 or 2 groups selected from halo, cyano, alkyl, haloalkyl, alkoxy, cycloalkyloxy, amino, alkylamino, and dialkylamino.

In certain embodiments, the compound of Formula I is that where R² is phenyl substituted with 1 or 2 groups selected from halo, cyano, alkyl, haloalkyl, alkoxy, and cycloalkyloxy. In certain embodiments, R² is phenyl substituted with 1 or 2 groups selected from halo and alkyl. In certain embodiments, R² is phenyl substituted with 1 or 2 groups selected from chloro, bromo, methyl, and ethyl. In certain embodiments, R² is phenyl substituted with 1 or 2 groups selected from chloro and methyl. In certain embodiments, R² is phenyl substituted with chloro, bromo, methyl, or ethyl. In certain embodiments, R² is phenyl substituted with chloro or methyl. In certain embodiments, R² is phenyl substituted with an alkyl group. In certain embodiments, R² is phenyl substituted with methyl or ethyl. In certain embodiments, R² is phenyl substituted with halo. In certain embodiments, R² is phenyl substituted with chloro, bromo, or fluoro. In certain embodiments, R² is phenyl substituted with fluoro. In certain embodiments, R² is phenyl substituted with chloro or bromo. In certain embodiments, R² is phenyl substituted with chloro. In certain embodiments, R² is phenyl substituted with bromo.

In certain embodiments, the compound of Formula I is that where R¹ and R² are the same.

In certain embodiments, the compound of Formula I is that where R¹ and R² are each substituted with one substituent in the ortho position. In certain embodiments, R¹ and R² are each substituted with one substituent in the meta position. In certain embodiments, R¹ and R² are each substituted with one substituent in the para position. In certain embodiments, R¹ and R² are each di-substituted in the ortho positions. In certain embodiments, R¹ and R² are each di-substituted in the meta positions. In certain embodiments, R¹ and R² are each di-substituted in the ortho and meta positions. In certain embodiments, R¹ and R² are each di-substituted in the ortho and para positions. In certain embodiments, R¹ and R² are each di-substituted in the meta and para positions. In certain embodiments, one of R¹ or R² is mono-substituted in the ortho position and the other is di-substituted in the ortho positions. In certain embodiments, one of R¹ or R² is mono-substituted in the ortho position and the other is di-substituted in the meta positions. In certain embodiments, one of R¹ or R² is mono-substituted in the meta position and the other is di-substituted in the ortho positions. In certain embodiments, one of R¹ or R² is mono-substituted in the meta position and the other is di-substituted in the meta positions. In certain embodiments, one of R¹ or R² is mono-substituted in the para position and the other is di-substituted in the ortho positions. In certain embodiments, one of R¹ or R² is mono-substituted in the para position and the other is di-substituted in the meta positions.

In certain embodiments, the compound of Formula I is that where R³ is alkyl, haloalkyl, hydroxyalkyl, alkoxycarbonyl, heterocycloalkyl, aryl, or heteroaryl; wherein the heterocycloalkyl, aryl, and heteroaryl groups are each optionally substituted. In certain embodiments, R³ is alkyl, haloalkyl, hydroxyalkyl, heterocycloalkyl, aryl, or heteroaryl; wherein the heterocycloalkyl, aryl, and heteroaryl groups are each optionally substituted. In certain embodiments, R³ is heterocycloalkyl, aryl, or heteroaryl; wherein each group is optionally substituted. In certain embodiments, R³ is alkyl, haloalkyl, hydroxyalkyl, alkoxycarbonyl, heterocycloalkyl, aryl, or heteroaryl; wherein the heterocycloalkyl, aryl, and heteroaryl groups are each optionally substituted. In certain embodiments, R³ is alkyl.

In certain embodiments, the compound of Formula I is that where R³ is aryl or heteroaryl; wherein each group is optionally substituted. In certain embodiments, R³ is optionally substituted phenyl. In certain embodiments, R³ is substituted phenyl. In certain embodiments, R³ is optionally substituted heteroaryl. In certain embodiments, R³ is pyridinyl, pyrrolyl, furanyl, thienyl, thiazolyl, oxazolyl, indolyl, benzofuranyl, benzothienyl, benzothiazolyl, or benzimidazoly; wherein each group is optionally substituted. In certain embodiments, R³ is pyridinyl, pyrrolyl, furanyl, thienyl, thiazolyl, oxazolyl, indolyl, benzofuranyl, benzothienyl, benzothiazolyl, or benzimidazoly; wherein each group is substituted. In certain embodiments, R³ is pyrrolidinyl, furanyl, thienyl, thiazolyl, pyridinyl, benzimidazolyl, benzofuranyl, benzothienyl, or benzothiazolyl; wherein each group is optionally substituted. In certain embodiments, R³ is pyrrolidinyl, furanyl, thienyl, thiazolyl, pyridinyl, benzimidazolyl, benzofuranyl, benzothienyl, or benzothiazolyl; wherein each group is substituted.

In certain embodiments, the compound of Formula I is that where R³ is optionally substituted with 1 or 2 groups selected from halo, cyano, hydroxy, alkyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo. In certain embodiments, R³ is optionally substituted with 1 or 2 groups selected from halo, cyano, alkyl, alkynyl, alkoxy, and haloalkoxy. In certain embodiments, R³ is optionally substituted with 1 or 2 groups selected from chloro, cyano, methyl, ethyl, methoxy, difluoromethoxy, and trifluoromethoxy.

In certain embodiments, the compound of Formula I is that where R³ is substituted with one substituent in the ortho position. In certain embodiments, R³ is substituted with one substituent in the meta position. In certain embodiments, R³ is substituted with one substituent in the para position. In certain embodiments, R³ is di-substituted in the ortho positions. In certain embodiments, R³ is di-substituted in the meta positions. In certain embodiments, R³ is di-substituted in the ortho and meta positions. In certain embodiments, R³ is di-substituted in the ortho and para positions. In certain embodiments, R³ is di-substituted in the meta and para positions.

In certain embodiments, the compound of Formula I is that where R³ is alkyl, haloalkyl, hydroxyalkyl, or alkoxycarbonyl. In certain embodiments, R³ is alkyl, haloalkyl, or hydroxyalkyl. In certain embodiments, R³ is hydrogen.

In certain embodiments, the compound of Formula I is that where R⁴ is hydrogen, alkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl. In certain embodiments, R⁴ is hydrogen, alkyl, cycloalkyl, heterocycloalkyl, phenyl, or thienyl. In certain embodiments, R⁴ is alkyl. In certain embodiments, R⁴ is alkyl, cycloalkyl, or phenyl. In certain embodiments, R⁴ is hydrogen or alkyl. In certain embodiments, R⁴ is cycloalkyl, heterocycloalkyl, phenyl, or thienyl. In certain embodiments, R⁴ is methyl, ethyl, propyl, or cyclopropyl. In certain embodiments, R⁴ is methyl, ethyl, or propyl. In certain embodiments, R⁴ is methyl or ethyl. In certain embodiments, R⁴ is methyl. In certain embodiments, R⁴ is hydrogen.

In certain embodiments, the compound of Formula I is that where R⁴ is hydrogen, alkyl, haloalkyl, cycloalkyl, heterocycloalkyl, phenyl, or thienyl; wherein the alkyl is optionally substituted with 1 or 2 groups selected from cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl. In certain embodiments, R⁴ is hydrogen, alkyl, haloalkyl, cycloalkyl, heterocycloalkyl, phenyl, or thienyl. In certain embodiments, R⁴ is haloalkyl. In certain embodiments, R⁴ is alkyl or haloalkyl. In certain embodiments, R⁴ is trifluoromethyl.

In certain embodiments, the compound of Formula I is that where R⁴ is substituted phenyl. In certain embodiments, R⁴ is phenyl substituted with one substituent in the ortho position. In certain embodiments, R⁴ is phenyl substituted with one substituent in the meta position. In certain embodiments, R⁴ is phenyl substituted with one substituent in the para position. In certain embodiments, R⁴ is phenyl di-substituted in the ortho positions. In certain embodiments, R⁴ is phenyl di-substituted in the meta positions. In certain embodiments, R⁴ is phenyl di-substituted in the ortho and meta positions. In certain embodiments, R⁴ is phenyl di-substituted in the ortho and para positions. In certain embodiments, R⁴ is phenyl di-substituted in the meta and para positions.

In certain embodiments, the compound of Formula I is that where R⁴ and R⁵ and the carbon to which they are attached combine to form spirocycloalkyl or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino. In certain embodiments, the compound of Formula I is that where R⁴ and R⁵ and the carbon to which they are attached combine to form spirocyclopropyl, spirocyclobutyl, spirocyclopentyl, spirooxetane, spirotetrahydrofuran, spiroazetidine, or spiropyrrolidine group, each optionally substituted with 1 or 2 groups selected from halo, cyano, hydroxy, C₁₋₃ alkyl, alkoxy, amino, alkylamino, and dialkylamino.

In certain embodiments, the compound of Formula I is that where R⁵ is hydrogen, alkyl, alkoxy, or cycloalkyl. In certain embodiments, R⁵ is hydrogen or alkyl. In certain embodiments, R⁵ is alkyl or cycloalkyl. In certain embodiments, R⁵ is hydrogen. In certain embodiments, R⁵ is alkyl. In certain embodiments, R⁵ is cycloalkyl.

In certain embodiments, the compound of Formula I is that where R⁴ is hydrogen, alkyl, cycloalkyl, heterocycloalkyl, phenyl, or thienyl; wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and R⁵ is hydrogen, alkyl, or cycloalkyl. In certain embodiments, R⁴ is hydrogen, alkyl, cycloalkyl, heterocycloalkyl, phenyl, or thienyl; wherein each is optionally substituted; and R⁵ is hydrogen or alkyl. In certain embodiments, R⁴ is alkyl and R⁵ is alkyl. In certain embodiments, R⁴ and R⁵ are hydrogen.

In certain embodiments, the compound of Formula I is that where R⁴ and R⁵ and the carbon to which they are attached combine to form carbonyl, spirocycloalkyl, or spiroheterocycloalkyl. In certain embodiments, R⁴ and R⁵ and the carbon to which they are attached, combine to form carbonyl or spirocycloalkyl. In certain embodiments, R⁴ and R⁵ and the carbon to which they are attached, combine to form carbonyl. In certain embodiments, R⁴ and R⁵ and the carbon to which they are attached, combine to form spirocycloalkyl. In certain embodiments, R⁴ and R⁵ and the carbon to which they are attached, combine to form spirocyclopropyl.

In certain embodiments, the compound of Formula I is not 1-(3-aminophenyl)-5-hydroxy-3-phenylimidazolidine-2,4-dione or 4-hydroxy-1,3,4,5-tetraphenylimidazolidin-2-one.

In certain embodiments, the compound of Formula I is that wherein R¹, R², R³ and R⁴ cannot all be unsubstituted phenyl. In certain embodiments, the compound of Formula I is that wherein when R⁴ and R⁵ and the carbon to which they are attached combine to form a carbonyl, and R¹ is phenyl substituted with amino, then R³ is not hydrogen

In certain embodiments, the compound of Formula I is not 1-(3-aminophenyl)-5-hydroxy-3-phenylimidazolidine-2,4-dione. In certain embodiments, when R¹ is 3-aminophenyl and R² is unsubstituted phenyl, then R³ is not H. In certain embodiments, when R¹ is 3-aminophenyl, R² is unsubstituted phenyl, and R⁴ and R⁵ and the carbon to which they are attached combine to form carbonyl, then R³ is not H. In certain embodiments, when R¹ is 3-aminophenyl and R⁴ and R⁵ and the carbon to which they are attached combine to form carbonyl, R² is substituted phenyl. In certain embodiments, when R⁴ and R⁵ and the carbon to which they are attached combine to form carbonyl, both R¹ and, R² are substituted phenyl. In certain embodiments, R¹ is not an amino-substituted phenyl.

In certain embodiments, the compound of Formula I is not 4-hydroxy-1,3,4,5-tetraphenylimidazolidin-2-one. In certain embodiments, when R¹ and R² are both unsubstituted phenyl, then neither R³ nor R⁴ is unsubstituted phenyl. In certain embodiments, when R¹ and R² are both unsubstituted phenyl, then R³ is not unsubstituted phenyl. In certain embodiments, when R¹ and R² are both unsubstituted phenyl, then R⁴ is not unsubstituted phenyl. In certain embodiments, when R¹ and R² are both unsubstituted phenyl and R⁵ is H, then neither R³ nor R⁴ is unsubstituted phenyl. In certain embodiments, when R¹ and R² are both unsubstituted phenyl and R⁵ is H, then R³ is not unsubstituted phenyl. In certain embodiments, when R¹ and R² are both unsubstituted phenyl and R⁵ is H, then R⁴ is not unsubstituted phenyl. In certain embodiments, when R³ and R⁴ are unsubstituted phenyl, at least one of R¹ and R² is substituted phenyl.

In certain embodiments, the compound of Formula I is that wherein when R⁴ and R⁵ and the carbon to which they are attached combine to form a carbonyl, and R¹ and R² are both unsubstituted phenyl, then R³ is not alkyl, haloalkyl, or aryl. In certain embodiments, the compound of Formula I is that wherein when R⁴ and R⁵ and the carbon to which they are attached combine to form carbonyl, and R¹ and R² are both unsubstituted phenyl, then R³ is not methyl, ethyl, tert-butyl, trifluoromethyl, pentafluoroethyl, unsubstituted phenyl, unsubstituted naphthyl, or unsubstituted anthracenyl. In certain embodiments, the compound of Formula I is that wherein when R¹ and R² are both unsubstituted phenyl, then R³ is not methyl, ethyl, tert-butyl, trifluoromethyl, pentafluoroethyl, unsubstituted phenyl, unsubstituted naphthyl, or unsubstituted anthracenyl. In certain embodiments, wherein when R⁴ and R⁵ and the carbon to which they are attached combine to form a carbonyl, and R³ is alkyl, haloalkyl, or aryl, then at least one of R¹ and R² is a substituted phenyl.

In certain embodiments, the compound of Formula I is that wherein when when R¹ and R² are both unsubstituted phenyl, R³ and R⁴ are both H, then R⁵ is not alkoxy.

In certain embodiments, the compound of Formula I is that wherein when when R¹ and R² are both unsubstituted or C₁-C₄ alkyl-substituted C₆-C₁₀ aryl, and R⁴ and R⁵ and the carbon to which they are attached combine to form carbonyl, then R³ is not H. In certain embodiments, when R¹ and R² are both unsubstituted or C₁-C₄ alkyl-substituted C₆-C₁₀ aryl, and R³ and R⁴ are both H, then R⁵ is not hydrogen, C₁-C₄ alkyl, C₃-C₁₂ cycloalkyl, C₁-C₄ alkoxy or unsubstituted or C₁-C₄ alkyl-substituted C₆-C₁₀ aryl.

In certain embodiments, the compound of Formula I is that according to Formula Ia:

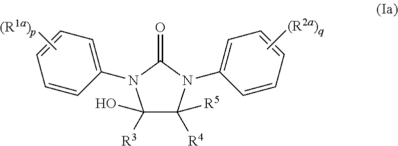

where all groups are as defined in the Summary or as defined in any of the embodiments described herein.

In certain embodiments, the compound of Formula I is that according to Formula Ia, wherein:
R^{1a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R^{2a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
p is 0, 1, or 2;
q is 0, 1, or 2; and
   where all other groups are as defined in the Summary or as defined in any of the embodiments described herein.

In certain embodiments, the compound of Formula I is that according to Formula Ib:

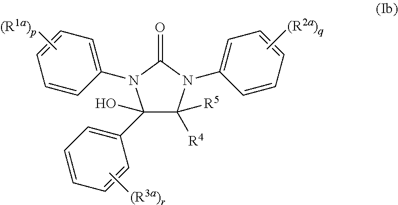

where all groups are as defined in the Summary or as defined in any of the embodiments described herein.

In certain embodiments, the compound of Formula I is that according to Formula Ib, wherein:
R^{3a} is selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
r is 0, 1, or 2; and
   where all other groups are as defined in the Summary or as defined in any of the embodiments described herein.

In certain embodiments, the compound of Formula I is that according to Formula Ic:

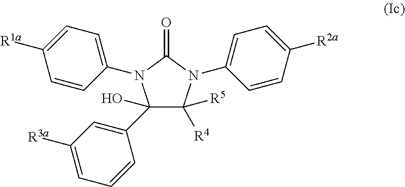

where all groups are as defined in the Summary or as defined in any of the embodiments described herein.

In certain embodiments, the compound of Formula I is that according to Formula Ic, wherein:
R^{1a} is selected from halo, cyano, alkyl, haloalkyl, alkoxy, cycloalkyloxy, amino, alkylamino, and dialkylamino;
R² is selected from halo, cyano, alkyl, haloalkyl, alkoxy, cycloalkyloxy, amino, alkylamino, and dialkylamino;
R³ is selected from halo, cyano, hydroxy, alkyl, alkenyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo;
R⁴ is hydrogen, alkyl, cycloalkyl, heterocycloalkyl, phenyl, or thienyl; and
R⁵ is hydrogen, alkyl, or cycloalkyl;
or R⁴ and R⁵ and the carbon to which they are attached combine to form carbonyl, or spirocycloalkyl.

In certain embodiments, the compound of Formula I is that according to Formula Ic, wherein:
R^{1a} is selected from halo, cyano, alkyl, haloalkyl, alkoxy, cycloalkyloxy, amino, alkylamino, and dialkylamino;
R² is selected from halo, cyano, alkyl, haloalkyl, alkoxy, cycloalkyloxy, amino, alkylamino, and dialkylamino;
R³ is selected from halo, cyano, hydroxy, alkyl, alkenyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo;
R⁴ is hydrogen, alkyl, haloalkyl, cycloalkyl, heterocycloalkyl, phenyl, or thienyl; and
R⁵ is hydrogen, alkyl, or cycloalkyl;
or R⁴ and R⁵ and the carbon to which they are attached combine to form carbonyl, or spirocycloalkyl.

In certain embodiments, the compound of Formula I is that according to Formula Ic, where R^{1a} is halo, cyano, alkyl, haloalkyl, or alkoxy; R^{2a} is halo, cyano, alkyl, haloalkyl, or alkoxy; R³ is halo, cyano, alkyl, haloalkyl, alkoxy, haloalkoxy, or phenyl optionally substituted with halo; R⁴ is hydrogen, alkyl, cycloalkyl, heterocycloalkyl, phenyl, or thienyl; and R⁵ is hydrogen, alkyl, or cycloalkyl; or R⁴ and R⁵ and the carbon to which they are attached combine to form carbonyl, or spirocycloalkyl.

In certain embodiments, the compound of Formula I is that according to Formula Ic, where R^{1a} is halo, cyano, alkyl, haloalkyl, or alkoxy; R^{2a} is halo, cyano, alkyl, haloalkyl, or alkoxy; R³ is halo, cyano, alkyl, haloalkyl, alkoxy, haloalkoxy, or phenyl optionally substituted with halo; R⁴ is hydrogen alkyl, or cycloalkyl; and R⁵ is hydrogen or alkyl; or R⁴ and R⁵ and the carbon to which they are attached combine to form spirocycloalkyl.

In certain embodiments, the compound of Formula I is that according to Formula Ic, where R^{1a} is halo, cyano, alkyl, or haloalkyl; R^{2a} is halo, cyano, alkyl, or haloalkyl; R³ is halo, cyano, alkyl, haloalkyl, haloalkoxy, or phenyl optionally substituted with halo; R⁴ is hydrogen alkyl, or cycloalkyl; and R⁵ is hydrogen or alkyl; or R⁴ and R⁵ and the carbon to which they are attached combine to form spirocycloalkyl.

In certain embodiments, the compound of Formula I is that according to Formula Ic, where R^{1a} is halo, cyano, alkyl, or haloalkyl; R^{2a} is halo, cyano, alkyl, or haloalkyl; R³ is halo, cyano, alkyl, or haloalkoxy; R⁴ is hydrogen alkyl, or cycloalkyl; and R⁵ is hydrogen or alkyl; or R⁴ and R⁵ and the carbon to which they are attached combine to form spirocycloalkyl.

In certain embodiments, the compound of Formula I is that according to Formula Ic, where R^{1a} is halo, cyano, alkyl, or haloalkyl; R^{2a} is halo, cyano, alkyl, or haloalkyl; R³ is halo, cyano, alkyl, or haloalkoxy; R⁴ is hydrogen alkyl, haloalkyl, or cycloalkyl; and R⁵ is hydrogen or alkyl; or R⁴ and R⁵ and the carbon to which they are attached combine to form spirocycloalkyl.

In certain embodiments, the compound of Formula I is that according to Formula Id:

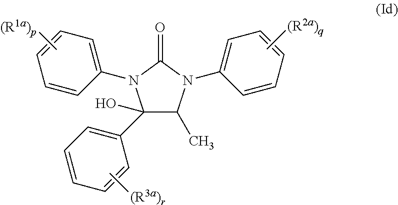

where all groups are as defined in the Summary or as defined in any of the embodiments described herein.

In certain embodiments, the compound of Formula I is that according to Formula Ie:

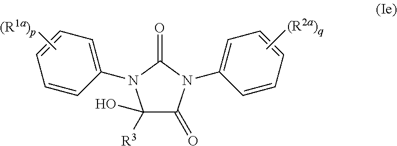

where all groups are as defined in the Summary or as defined in any of the embodiments described herein.

In certain embodiments, the compound of Formula I is that according to Formula If:

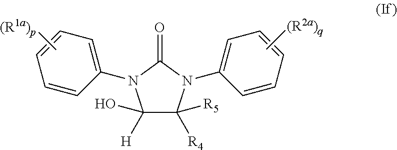

where all groups are as defined in the Summary or as defined in any of the embodiments described herein.

In certain embodiments, the compound of Formula I is that according to one of the following formulas:

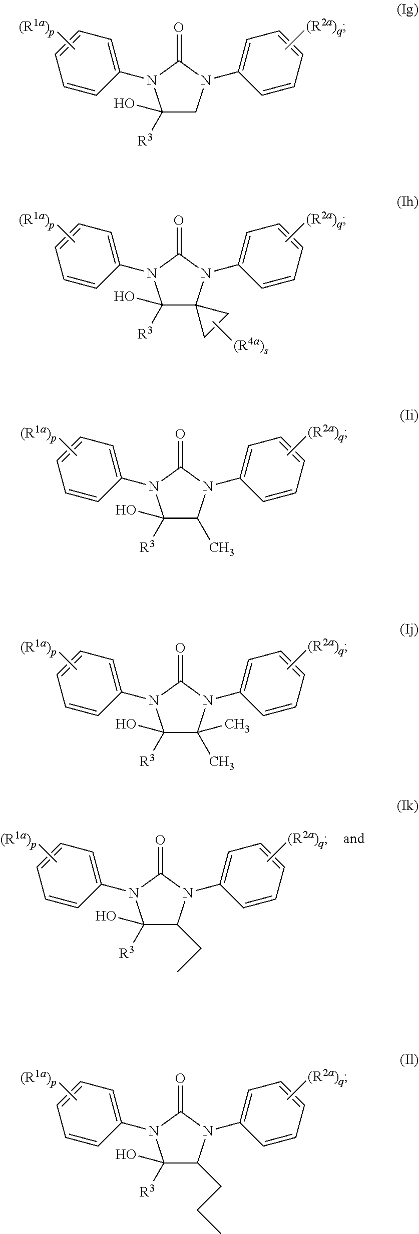

wherein:
R^{4a} is selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
s is 0, 1, or 2; and
   where all other groups are as defined in the Summary or as defined in any of the embodiments described herein.

In certain embodiments, the compound of Formula I is that according to one of the following formulas:

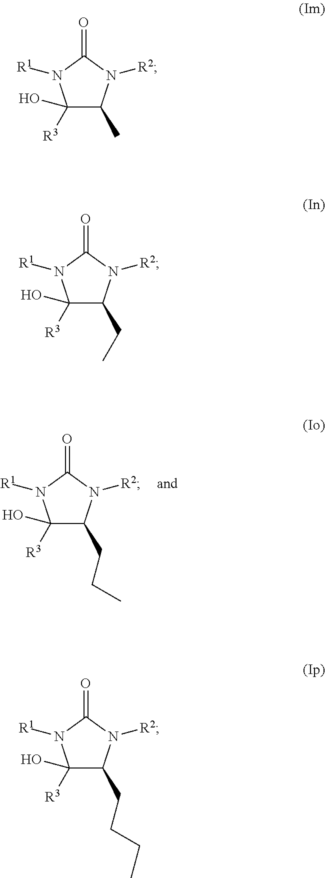

where all groups are as defined in the Summary or as defined and/substituted in any of the embodiments described herein.

In certain embodiments, the compound of Formula I is that according to one of the following formulas:

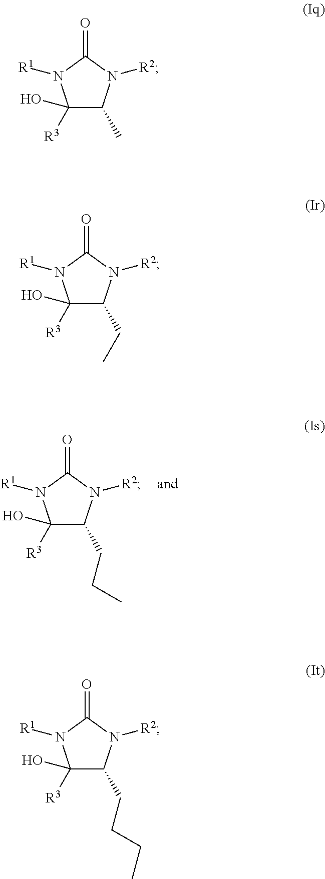

where all groups are as defined in the Summary or as defined and/substituted in any of the embodiments described herein.

In certain embodiments, the compound of Formula I is that according to one of the following formulas:

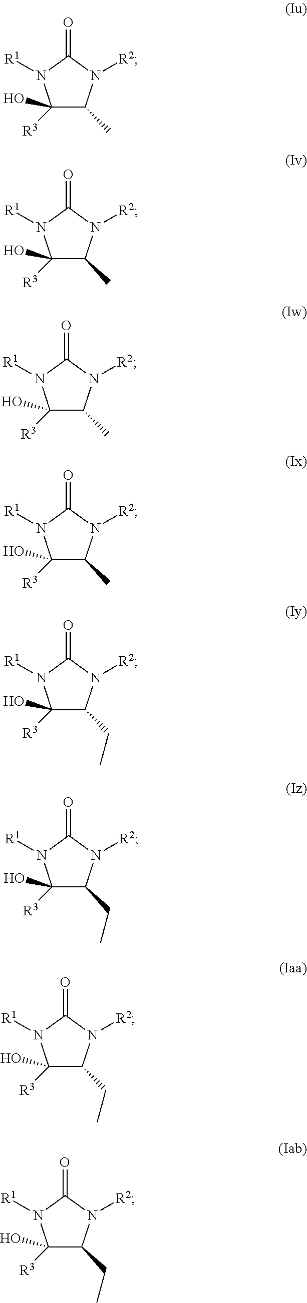

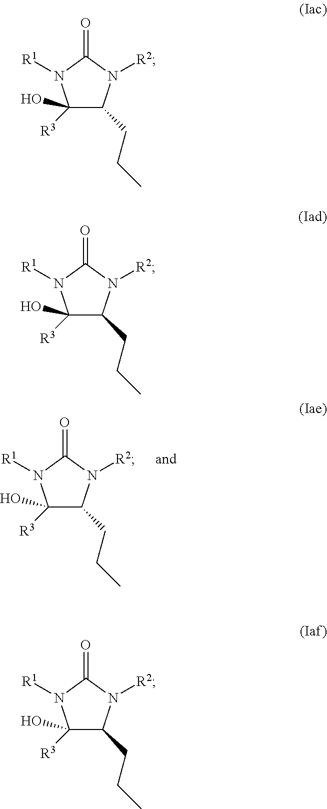

where all groups are as defined in the Summary or as defined and/substituted in any of the embodiments described herein.

In another embodiment, provided is a compound of Formula II:

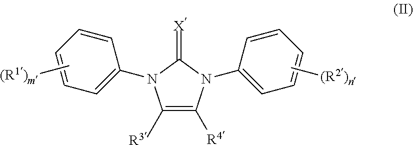

wherein:
X′ is O or S;
R^{1′} is halo, cyano, alkyl, haloalkyl, or alkoxy;
R^{2′} is halo, cyano, alkyl, haloalkyl, or alkoxy;
R^{3′} is alkyl, phenyl, heteroaryl with 5-6 ring atoms, or phenylcarbonyl, wherein the phenyl, heteroaryl, or phenylcarbonyl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl;
R^{4′} is hydrogen, alkyl, alkoxyalkyl, C₃₋₅ cycloalkyl, or 3-6 membered heterocycloalkyl, wherein the cycloalkyl and heterocycloalkyl groups are each independently optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R^{3′} and R^{4′} and the carbons to which they are attached combine to form a 5-6 membered cycloalkylene;
m′ is 1 or 2; and
n′ is 1 or 2;
with the proviso that when R^{1′} and R^{2′} are each methoxy, then R^{3′} cannot be methoxy-substituted phenyl;
   or a pharmaceutically acceptable salt thereof.

In certain embodiments, the compound of Formula II is that wherein:
X′ is O;
R^{1′} and R^{2′} are each independently selected from halo;
R^{3′} is alkyl, phenyl, heteroaryl with 5 ring atoms, or phenylcarbonyl, wherein the phenyl, heteroaryl, or phenylcarbonyl are each optionally substituted with a group selected from halo, cyano, alkyl, haloalkyl, hydroxyalkyl, haloalkoxy, and alkylcarbonyl.
R^{4′} is hydrogen, alkyl, alkoxyalkyl, C₃₋₅ cycloalkyl, or 3-6 membered heterocycloalkyl;
or R^{3′} and R^{4′} and the carbons to which they are attached combine to form a 5-6 membered cycloalkylene; and
m′ and n′ are 1.

In certain embodiments, the compound of Formula II is that where X′ is S.

In certain embodiments, the compound of Formula II is that where X′ is O.

In certain embodiments, the compound of Formula II is that where R^{1′} and R^{2′} are each independently selected from halo. In certain embodiments, R^{1′} and R^{2′} are each independently selected from chloro and bromo. In certain embodiments, R^{1′} and R^{2′} are each chloro. In certain embodiments, R^{1′} and R^{2′} are each bromo.

In certain embodiments, the compound of Formula II is that where R^{1′} and R^{2′} are each independently selected from halo and alkyl. In certain embodiments, R^{1′} and R^{2′} are each independently selected from halo and methyl.

In certain embodiments, the compound of Formula II is that where R^{1′×} and R^{2′} are each independently selected from halo, cyano, haloalkyl, and alkoxy. In certain embodiments, R^{1′} and R^{2′} are each independently selected from halo, cyano, and haloalkyl. In certain embodiments, R^{1′} and R^{2′} are each independently selected from halo and cyano.

In certain embodiments, the compound of Formula II is that where R^{1′} and R^{2′} are at the para position.

In certain embodiments, the compound of Formula II is that where R^{3′} is alkyl, optionally substituted phenyl, or optionally substituted heteroaryl with 5-6 ring atoms. In certain embodiments, R^{3′} is optionally substituted phenyl, or optionally substituted heteroaryl with 5-6 ring atoms. In certain embodiments, R^{3′} is optionally substituted phenyl. In certain embodiments, R^{3′} is phenyl substited with halo, cyano, alkyl, haloalkyl, hydroxyalkyl, haloalkoxy, or alkylcarbonyl. In certain embodiments, R^{3′} is phenyl. The compound of claim 1, wherein R^{3′} is optionally substituted heteroaryl.

In certain embodiments, the compound of Formula II is that where R^{4′} is alkyl, alkoxyalkyl, C₃₋₅ cycloalkyl, or 3-6 membered heterocycloalkyl.

In certain embodiments, the compound of Formula II is that where R^{4′} is hydrogen, alkyl, or C₃₋₅ cycloalkyl. In certain embodiments, R^{4′} is alkyl or C₃₋₅ cycloalkyl. In certain embodiments, the R^{4′} is hydrogen.

In certain embodiments, the compound of Formula II is that wherein when R^{1′} and R^{2′} are each methoxy, and R^{4′} is methyl, then R^{3′} is not methoxy-substituted phenyl. In certain embodiments, the compound of Formula II is that wherein when R^{1′} and R^{2′} are each methoxy, and R^{4′} is methyl, then R^{3′} is not mono substituted phenyl. In certain embodiments, the compound of Formula II is that wherein when R^{1′} and R^{2′} are each methoxy, and R^{4′} is methyl, then R^{3′} is not substituted phenyl. In certain embodiments, the compound of Formula II is that wherein when R^{3′} is methoxy-substituted phenyl, then at least one of R^{1′} and R^{2′} is not methoxy. In certain embodiments, the compound of Formula II is that wherein when R^{3′} is methoxy-substituted phenyl, then at least one of R^{1′} and R^{2′} is not alkoxy.

In certain embodiments, the compound of Formula II is that according to Formula IIa:

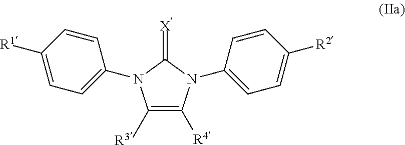

where all groups are as defined in the Summary or as defined in any of the embodiments described herein.

In certain embodiments, the compound of Formula II is that according to Formula IIb:

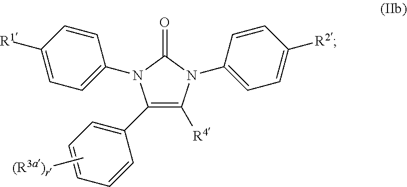

wherein:
R^{3a′} is selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl;
r′ is 0, 1, or 2; and
   where all other groups are as defined in the Summary or as defined in any of the embodiments described herein.

In certain embodiments, the compound of Formula II is that according to one of the following formulas:

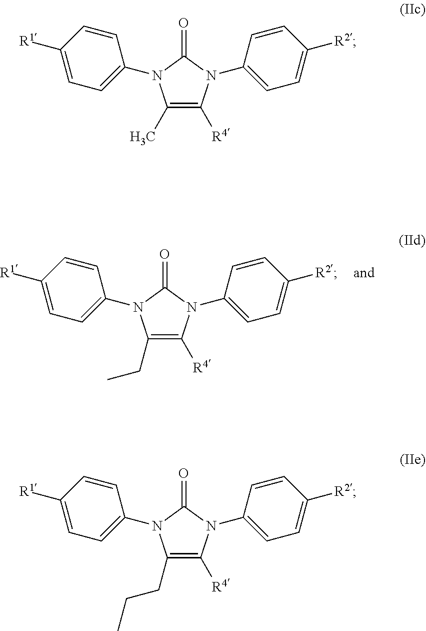

where all groups are as defined in the Summary or as defined in any of the embodiments described herein.

In certain embodiments, the compound is selected from Table 1.

In certain embodiments, the compound is selected from the group consisting of the compounds of Table 1 consisting of Examples 1-267, or a single stereoisomer or mixture of stereoisomers thereof.

In certain embodiments, the compound is selected from the group consisting of the compounds of Table 1 consisting of Examples 1-141 and 143-267, or a single stereoisomer or mixture of stereoisomers thereof.

In certain embodiments, the compound is selected from the group consisting of the compounds of Table 1 consisting of Examples 268-338, or a single stereoisomer or mixture of stereoisomers thereof.

In certain embodiments, the compound is a compound of Formula I, or a single stereoisomer or mixture of stereoisomers thereof, as defined herein.

In certain embodiments, the compound is a compound of Formula Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Iab, lac, lad, Iae, or Iaf, or a single stereoisomer or mixture of stereoisomers thereof, as defined herein. In certain embodiments, the compound is selected from the group consisting of the compounds of Table 1 consisting of Examples of 1-12, 13, 13-1, 13-2, 14-24, 25, 25-1, 25-2, 26-28, 29-1, 29-2, 30-35, 36, 36-1, 36-2, 37-41, 42, 42-1, 42-2, 43, 44, 45, 45-1, 45-2, 46-48, 49, 49-1, 49-2, 50, 51, 51-1, 51-2, 52, 53-1, 53-2, 54-61, 62, 62-1, 62-2, 63-65, 66, 66-1, 66-2, 67, 67-1, 67-2, 68, 68-1, 68-2, 69, 69-1, 69-2, 70, 71-1, 71-2, 72, 73, 73-1, 73-2, 74-1, 74-2, 75-1, 75-2, 76-1, 76-2, 77-1, 77-2, 78, 79, 80-1, 80-2, 81, 81-1, 81-2, 82, 82-1, 82-2, 83, 83-1, 83-2, 84-1, 84-2, 85, 86-1, 86-2, 87, 87-1, 87-2, 88, 88-1, 88-2, 89, 89-1, 89-2, 90, 90-1, 90-2, 91-1, 91-2, 92-1, 92-2, 93, 93-1, 93-2, 94, 94-1, 94-2, 95, 96, 96-1, 96-2, 97, 97-1, 97-2, 98, 99, 99-1, 99-2, 100-1, 100-2, 101, 101-1, 101-2, 102, 103, 103-1, 103-2, 104, 104-1, 104-2, 105, 105-1, 105-2, 106, 106-1, 106-2, 107, 108, 109, 110, 111, 112-1, 112-2, 113, 114, 114-1, 114-2, 115, 115-1, 115-2, 116, 116-1, 116-2, 117-1, 117-2, 118, 118-1, 118-2, 119, 119-1, 119-2, 120, 121, 121-1, 121-2, 121-3, 121-4, 122, 123-1, 123-2, 124, 125, 126, 126-1, 126-2, 127, 128, 129, 129-1, 129-2, 130, 130-1, 130-2, 131, 132, 132-1, 132-2, 133-135, 136-1, 136-2, 137, 138, 139, 139-1, 139-2, 140, 140-1, 140-2, 141, 143-148, 149, 149-1, 149-2, 150-158, 159, 159-1, 159-2, 160-162, 161, 163-1, 163-2, 164, 165, 166, 166-1, 166-2, 167-169, 170, 170-1, 170-2, 171-176, 177, 177-1, 177-2, 178, 179, 180, 180-1, 180-2, 181-184, 185-1, 185-2, 186, 187-1, 187-2, 188, 189, 190, 190-1, 190-2, 191, 191-1, 191-2, 192, 193, 194, 194-1, 194-2, 195, 196, 196-1, 196-2, 197-208, 209-1, 209-2, 210-217, 218, 218-1, 218-2, 219-1, 219-2, 220, 221, 221-2, 221-1, 222, 222-1, 222-2, 223, 223-1, 223-2, 224-229, 230, 230-1, 230-2, 231, 232, 233, 233-1, 233-2, 234, 234-1, 234-2, 234-3, 234-4, 235, 235-1, 235-2, 236, 237, 238-1, 238-2, and 239-267, or a single stereoisomer or mixture of stereoisomers thereof.

In certain embodiments, the compound is selected from the group consisting of the compounds of Table 1 consisting of Examples 268-295, 296, 296-1, 296-2, 296-3, 296-4, 297, 297-1, 297-2, 298-318, 319-1, 319-2, 320-1, 320-2, 321-323, 324-1, 324-2, 325-328, 329, 329-1, 329-2, 330, 331, 331-1, 331-2, and 332-338, or a single stereoisomer or mixture of stereoisomers thereof.

In certain embodiments, the compound is selected from the group consisting of the compounds of Table 1 consisting of Examples 272B, 290B, 291A, 305A, 306H, 319, 320, 321E, 324A, 324B, 331A, 332A, and 336B, or a single stereoisomer or mixture of stereoisomers thereof.

In certain embodiments, the compound is selected from the group consisting of the compounds of Table 1 consisting of Examples 1-2, 4-17, 19-21, 23-26, 28-36, 38-39, 42-43, 45, 47, 49, 51-64, 66-69, 71-242, or a single stereoisomer or mixture of stereoisomers thereof.

In certain embodiments, the compound is selected from the group consisting of the compounds of Table 1 consisting of Examples 1-2, 4-17, 19-21, 23-26, 28-36, 38-39, 42-43, 45, 47, 49, 51-64, 66-69, 71-141, and 143-242, or a single stereoisomer or mixture of stereoisomers thereof.

In certain embodiments, the compound is selected from the group consisting of the compounds of Table 1 consisting of Examples 1-2, 4-17, 19-21, 23-26, 28-36, 38-39, 42-43, 45, 47, 49, 51-64, 66-69, 71-125, 127-140, 146, 150-151, 158-159, 163, 165-166, 170, 176-177, 180, 185-189, 191-192, 194, 196, 212, 217-218, 221, 224-230, and 233-242, or a single stereoisomer or mixture of stereoisomers thereof.

In certain embodiments, the compound is selected from the group consisting of the compounds of Table 1 consisting of Examples 126, 141-145, 147-149, 152-157, 160-162, 164, 167-169, 171-175, 178-179, 181-184, 190, 193, 195, 197-211, 213-216, 219-220, 222-223, and 231-232, or a single stereoisomer or mixture of stereoisomers thereof.

In certain embodiments, the compound is selected from the group consisting of the compounds of Table 1 consisting of Examples 126, 141, 143-145, 147-149, 152-157, 160-162, 164, 167-169, 171-175, 178-179, 181-184, 190, 193, 195, 197-211, 213-216, 219-220, 222-223, and 231-232, or a single stereoisomer or mixture of stereoisomers thereof.

In certain embodiments, the compound is selected from the group consisting of the compounds of Table 1 consisting of Examples 3, 13, 19, 25, 31, 36, 39, 40, 41, 42, 44, 45, 46, 48, 50, 51, 53, 58, 60, 62, 63, 65, 66, 67, 68, 69, 70, 71, 72, 73, 74, 75, 76, 77, 80, 81, 82, 83, 87, 88, 89, 90, 91, 92, 93, 94, 96, 99, 100, 101, 103, 104, 105, 106, 107, 110, 114, 115, 116, 118, 119, 120, 123, 124, 126, 129, 130, 131, 132, 133, 134, 135, 136, 137, 138, 139, 146, 149, 150, 151, 158, 159, 163, 165, 166, 170, 176, 177, 180, 185, 186, 187, 188, 189, 190, 191, 191, 192, 194, 196, 209, 212, 219, 220, 221, 222, 222, 223, 224, 225, 226, 228, 229, 233, 234, 235, 236, 237, 238, 239, 240, 241, 242, 245, 246, 247, 248, 249, 250, 257, 258, 264, and 267, or a single stereoisomer or mixture of stereoisomers thereof.

In certain embodiments, the compound is a compound of Formula II, as defined herein.

In certain embodiments, the compound is a compound of Formula IIa, IIb, IIc, IId, or IIe, as defined herein.

In certain embodiments, the compound is selected from the group consisting of the compounds of Table 1 consisting of Examples 3, 18, 22, 27, 37, 40, 41, 44, 46, 48, 50, 65, 70, and 243-267.

In certain embodiments, the compound is selected from the group consisting of the compounds of Table 1 consisting of Examples 3, 40, 41, 44, 46, 48, 50, 65, 70, 245, 246, 247, 248, 249, 250, 257, 258, 264, and 267.

### Compound of Formula VII

In another aspect, provided is a compound of Formula VII:

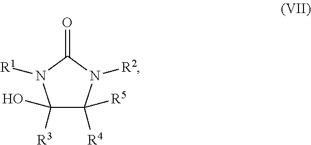

wherein:
R¹ is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is aryl optionally substituted with a group selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, cycloalkyl, or aryl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 groups selected from halo, cyano, hydroxy, C₁₋₃ alkyl, alkoxy, amino, alkylamino, and dialkylamino; and
provided that R⁴ and R⁵ are not both hydrogen; and
provided that the compound is not (4S,5S)-4-(tert-butyl)-1,3-bis(4-methoxyphenyl)-5-phenylimidazolidin-2-one; and
   optionally a single stereoisomer or mixture of stereoisomers thereof and additionally optionally a pharmaceutically acceptable salt thereof.

In certain embodiments, provided is a compound of Formula VII:

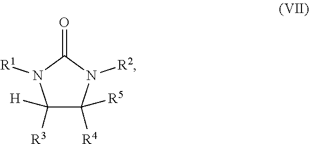

wherein:
R¹ is aryl optionally substituted with a group selected from halo, cyano, nitro, alkyl, alkenyl, haloalkoxy, haloalkyl, hydroxyalkyl, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with a group selected from halo, cyano, nitro, alkyl, alkenyl, haloalkoxy, haloalkyl, hydroxyalkyl, amino, alkylamino, dialkylamino, alkylcarbonyl, alkoxycarbonyl, aminocarbonyl, alkylaminocarbonyl, dialkylaminocarbonyl, and alkylcarbonylamino;
R³ is aryl optionally substituted with a group selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, cycloalkyl, or aryl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 groups selected from halo, cyano, hydroxy, C₁₋₃ alkyl, alkoxy, amino, alkylamino, and dialkylamino; and
provided that R⁴ and R⁵ are not both hydrogen; or provided that the compound is not (4S,S5S)-4-(tert-butyl)-1,3-bis(4-methoxyphenyl)-5-phenylimidazolidin-2-one; and
   optionally a single stereoisomer or mixture of stereoisomers thereof and additionally optionally a pharmaceutically acceptable salt thereof.

In certain embodiments, provided is a compound of Formula VII:

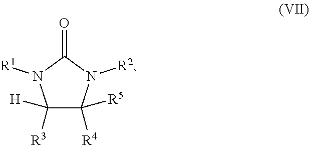

wherein:
R¹ is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with a group selected from halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is aryl optionally substituted with a group selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, cycloalkyl;
or R⁵ is aryl, when R³ and R⁴, together with the carbon atoms to which they are attached combine to form a C₅₋₆ cycloalkyl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 groups selected from halo, cyano, hydroxy, C₁₋₃ alkyl, alkoxy, amino, alkylamino, and dialkylamino; and
provided that R⁴ and R⁵ are not both hydrogen; or
provided that the compound of Formula VII is not (4S,5S)-4-(tert-butyl)-1,3-bis(4-methoxyphenyl)-5-phenylimidazolidin-2-one; or
provided that the compound of Formula VII is that wherein when R³ and R⁴, together with the carbon atoms to which they are attached combine to form a C₅₋₆ cycloalkyl or a 5-6 membered heterocycloalkyl, then the C₅₋₆ cycloalkyl or the 5-6 membered heterocycloalkyl ring atoms are not substituted with an oxo (═O) group; or
provided that the compound of Formula VII is that wherein when R³ and R⁴, together with the carbon atoms to which they are attached combine to form a 5-6 membered heterocycloalkyl, then the 5-6 membered heterocycloalkyl ring does not comprise —S(O)ₙ— (n is 0, 1, or 2); or
provided that the compound of Formula VII is that wherein when R³ and R⁴, together with the carbon atoms to which they are attached combine to form a C₆ cycloalkyl, then the cycloalkyl is saturated; or
provided that the compound of Formula VII is that wherein when R³ and R⁴, together with the carbon atoms to which they are attached combine to form a C₆ cycloalkyl or a 6-membered heterocycloalkyl, then R¹ and R² are both mono-substituted aryl; or
provided that the compound of Formula VII is that wherein when R³ and R⁴, together with the carbon atoms to which they are attached combine to form a C₆ cycloalkyl, or a 6-membered heterocycloalkyl, then the C₆ cycloalkyl or the 6-membered heterocycloalkyl is saturated and R¹ and R² are both mono-substituted aryl; and
   optionally a single stereoisomer or mixture of stereoisomers thereof and additionally optionally a pharmaceutically acceptable salt thereof.

In certain embodiments, the compound of Formula VII or pharmaceutically acceptable salt thereof is that wherein R¹ is aryl optionally substituted with a group selected from alkyl, alkenyl, cyano, halo, and haloalkyl;
R² is aryl optionally substituted with a group selected from alkyl, alkenyl, cyano, halo, and haloalkyl;
R³ is aryl optionally substituted with a group selected from alkyl, alkenyl, cyano, halo, haloalkyl, alkoxy, and haloalkoxy;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, or aryl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and
provided that R⁴ and R⁵ are not both hydrogen; and optionally a single stereoisomer or mixture of stereoisomers thereof and additionally optionally a pharmaceutically acceptable salt thereof.

In certain embodiments, the compound or salt of Formula VII is that wherein:
R¹ is aryl optionally substituted with a group selected from alkyl, alkenyl, cyano, halo, and haloalkyl;
R² is aryl optionally substituted with a group selected from alkyl, alkenyl, cyano, halo, and haloalkyl;
R³ is aryl optionally substituted with a group selected from alkyl, alkenyl, cyano, halo, haloalkyl, alkoxy, and haloalkoxy;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, or alkoxyalkylene, or aryl, wherein the aryl is optionally substituted with a group selected from alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and
provided that R⁴ and R⁵ are not both hydrogen; and
   optionally a single stereoisomer or mixture of stereoisomers thereof and additionally optionally a pharmaceutically acceptable salt thereof.

In certain embodiments, the compound or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ is aryl optionally substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen, alkyl, haloalkyl, or alkoxyalkylene; R⁵ is hydrogen, alkyl, haloalkyl, alkoxyalkylene, or aryl, wherein the aryl is optionally substituted with a group selected from cyano, haloalkyl, hydroxy, and haloalkoxy; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and provided that R⁴ and R⁵ are not both hydrogen; and optionally a single stereoisomer or mixture of stereoisomers thereof and additionally optionally a pharmaceutically acceptable salt thereof.

In certain embodiments, the compound or salt of Formula VII is that wherein: R¹ is aryl substituted with a group selected from alkyl, cyano, halo, and haloalkyl; R² is aryl substituted with a group selected from alkyl, cyano, halo, and haloalkyl; R³ is aryl substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen, alkyl, haloalkyl, or alkoxyalkylene; R⁵ is hydrogen, alkyl, haloalkyl, alkoxyalkylene, or aryl, wherein the aryl is substituted with a group selected from cyano, haloalkyl, hydroxy, and haloalkoxy; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and provided that R⁴ and R⁵ are not both hydrogen; and optionally a single stereoisomer or mixture of stereoisomers thereof and additionally optionally a pharmaceutically acceptable salt thereof.

In certain embodiments, the compound or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ is aryl, wherein the aryl group is optionally substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen, alkyl, haloalkyl, or alkoxyalkylene; R⁵ is hydrogen, alkyl, haloalkyl, or alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and provided that R⁴ and R⁵ are not both hydrogen; and optionally a single stereoisomer or mixture of stereoisomers thereof and additionally optionally a pharmaceutically acceptable salt thereof.

In certain embodiments, the compound or salt of Formula VII is that wherein: R¹ is aryl substituted with a group selected from alkyl, cyano, halo, and haloalkyl; R² is aryl substituted with a group selected from alkyl, cyano, halo, and haloalkyl; R³ is aryl, wherein the aryl group is substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen, alkyl, haloalkyl, or alkoxyalkylene; R⁵ is hydrogen, alkyl, haloalkyl, or alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and provided that R⁴ and R⁵ are not both hydrogen; and optionally a single stereoisomer or mixture of stereoisomers thereof and additionally optionally a pharmaceutically acceptable salt thereof.

In certain embodiments, the compound or salt or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ is aryl, wherein the aryl group is optionally substituted with a group selected from alkyl, cyano, halo, haloalkyl, and haloalkoxy; R⁴ is hydrogen, alkyl, haloalkyl, or alkoxyalkylene; R⁵ is hydrogen, alkyl, haloalkyl, or alkoxyalkylene; and provided that R⁴ and R⁵ are not both hydrogen.

In certain embodiments, the compound or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ is aryl, wherein the aryl group is optionally substituted with a group selected from alkyl, cyano, halo, haloalkyl, and haloalkoxy; and R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s).

In certain embodiments, the compound or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ and R⁴, together with the carbon atoms to which they are attached combine to form a C₅₋₆ cycloalkyl or a 5-6 membered heterocycloalkyl; and R⁵ is hydrogen or aryl, wherein the aryl is optionally substituted with a group selected from cyano, haloalkyl, hydroxy, and haloalkoxy.

In certain embodiments, the compound or salt of Formula VII is that wherein: R¹ is aryl optionally substituted with a group selected from alkyl, cyano, halo, and haloalkyl; R² is aryl optionally substituted with a group selected from alkyl, cyano, halo, and haloalkyl; R³ and R⁴, together with the carbon atoms to which they are attached combine to form a C₅₋₆ cycloalkyl or a 5-6 membered heterocycloalkyl; and R⁵ is aryl, wherein the aryl is optionally substituted with a group selected from cyano, haloalkyl, hydroxy, and haloalkoxy. In certain embodiments, R¹ is aryl optionally substituted with a group selected from alkyl, cyano, halo, and haloalkyl; R² is aryl optionally substituted with a group selected from alkyl, cyano, halo, and haloalkyl; R³ and R⁴, together with the carbon atoms to which they are attached combine to form a C₅₋₆ cycloalkyl or a 5-6 membered heterocycloalkyl; and R⁵ is aryl, wherein the aryl is optionally substituted with a group selected from cyano, haloalkyl, hydroxy, and haloalkoxy, provided that R¹ and R² are same.

In certain embodiments, the compound or salt of Formula VII is that wherein: R¹ is phenyl optionally substituted with a group selected from alkyl, cyano, halo, and haloalkyl; R² is phenyl optionally substituted with a group selected from alkyl, cyano, halo, and haloalkyl; R³ is phenyl optionally substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen, alkyl, haloalkyl, or alkoxyalkylene; R⁵ is hydrogen, alkyl, haloalkyl, alkoxyalkylene, or phenyl, wherein the phenyl is optionally substituted with a group selected from cyano, haloalkyl, hydroxy, and haloalkoxy; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s); and provided that R⁴ and R⁵ are not both hydrogen.

In certain embodiments, the compound or salt of Formula VII is that wherein: R¹ is phenyl optionally substituted with a group selected from alkyl, cyano, halo, and haloalkyl; R² is phenyl optionally substituted with a group selected from alkyl, cyano, halo, and haloalkyl; R³ is phenyl optionally substituted with a group selected from alkyl, cyano, halo, haloalkyl, and haloalkoxy; or R³ and R⁴, together with the carbon atoms to which they are attached combine to form a C₅₋₆ cycloalkyl or a 5-6 membered heterocycloalkyl; R⁴ is hydrogen or alkyl; and R⁵ is alkyl, haloalkyl, alkoxyalkylene, or phenyl, wherein the phenyl is optionally substituted with a group selected from cyano, haloalkyl, hydroxy, and haloalkoxy; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s).

In certain embodiments, the compound or salt of Formula VII is that wherein: R¹ and R², each independently, are phenyl optionally substituted with alkyl, cyano, halo, or haloalkyl; R³ is phenyl optionally substituted with alkyl, cyano, halo, haloalkyl, or haloalkoxy; R⁴ and R⁵, each independently, are selected from hydrogen, alkyl, haloalkyl, and alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups; and provided that R⁴ and R⁵ are not both hydrogen.

In certain embodiments, the compound or salt of Formula VII is that wherein: R¹ and R², each independently, are phenyl substituted with alkyl, cyano, halo, or haloalkyl; R³ is phenyl substituted with alkyl, cyano, halo, haloalkyl, or haloalkoxy; R⁴ and R⁵, each independently, are selected from hydrogen, alkyl, haloalkyl, and alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups; and provided that R⁴ and R⁵ are not both hydrogen. In certain embodiments, R¹ and R², each independently, are selected from phenyl substituted with alkyl, cyano, halo, or haloalkyl; R³ is unsubstituted phenyl; R⁴ and R⁵, each independently, are selected from hydrogen, alkyl, haloalkyl, and alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups; and provided that R⁴ and R⁵ are not both hydrogen. In certain embodiments, R¹ and R², each independently, are unsubstituted phenyl; R³ is phenyl substituted with alkyl, cyano, halo, haloalkyl, or haloalkoxy; R⁴ and R⁵, each independently, are selected from hydrogen, alkyl, haloalkyl, and alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups; and provided that R⁴ and R⁵ are not both hydrogen. In certain embodiments, R¹, R², R³, each independently, are unsubstituted phenyl; R⁴ and R⁵, each independently, are selected from hydrogen, alkyl, haloalkyl, and alkoxyalkylene; or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups; and provided that R⁴ and R⁵ are not both hydrogen. In certain embodiments, R¹ is substituted phenyl; R² is unsubstituted phenyl; and R³ is unsubstituted phenyl. In certain embodiments, the compound or salt of Formula VII is that wherein: R¹ is unsubstituted phenyl; R² is substituted phenyl; and R³ is unsubstituted phenyl. In certain embodiments, the compound or salt of Formula VII is that wherein: R¹ is substituted phenyl; R² is unsubstituted phenyl; and R³ is substituted phenyl. In certain embodiments, the compound or salt of Formula VII is that wherein: R¹ is unsubstituted phenyl; R² is substituted phenyl; and R³ is substituted phenyl.

In certain embodiments, the compound or salt of Formula VII is that wherein: R¹ and R², each independently, are selected from phenyl optionally substituted with alkyl, cyano, halo, or haloalkyl; R³ is phenyl optionally substituted with alkyl, cyano, halo, haloalkyl, or haloalkoxy; R⁴ and R⁵, each independently, are selected from hydrogen, alkyl, haloalkyl, and alkoxyalkylene; and provided that R⁴ and R⁵ are not both hydrogen. In certain embodiments, R¹ and R², each independently, are selected from phenyl optionally substituted with alkyl, cyano, halo, or haloalkyl; R³ is phenyl optionally substituted with alkyl, cyano, halo, haloalkyl, or haloalkoxy; R⁴ and R⁵, each independently, are selected from alkyl, haloalkyl, and alkoxyalkylene; and provided that R⁴ and R⁵ are not both hydrogen. In certain embodiments, R¹, R², R³, each independently, are optionally substituted phenyl; R⁴ and R⁵, each independently, are selected from hydrogen and alkyl; and provided that R⁴ and R⁵ are not both hydrogen. In certain embodiments, R¹, R², R³, each independently, are optionally substituted phenyl; and R⁴ and R⁵, each independently, are selected from alkyl and haloalkyl. In certain embodiments, R¹, R², R³, each independently, are optionally substituted phenyl; and R⁴ and R⁵, each independently, are selected from alkyl and haloalkoxy. In certain embodiments, R¹ and R², each independently, are selected from phenyl optionally substituted with alkyl, cyano, halo, or haloalkyl; R³ is phenyl optionally substituted with alkyl, cyano, halo, haloalkyl, or haloalkoxy; R⁴ is hydrogen or alkyl; and R⁵ is alkyl, haloalkyl, or alkoxyalkylene.

In certain embodiments, the compound or salt of Formula VII is that wherein: R¹ and R², each independently, are selected from phenyl optionally substituted with alkyl, cyano, halo, or haloalkyl; R³ is phenyl optionally substituted with alkyl, cyano, halo, haloalkyl, or haloalkoxy; R⁴ is hydrogen; and R⁵ is alkyl, haloalkyl, or alkoxyalkylene. In certain embodiments, R¹, R², R³, each independently, are optionally substituted phenyl; R⁴ is hydrogen; and R⁵ is alkyl. In certain embodiments, R¹, R², R³, each independently, are optionally substituted phenyl; R⁴ is hydrogen; and R⁵ is haloalkyl. In certain embodiments, R¹, R², R³, each independently, are optionally substituted phenyl; R⁴ is hydrogen; and R⁵ is alkoxyalkylene.

In certain embodiments, the compound or salt of Formula VII is that wherein: R¹ and R², each independently, are selected from phenyl optionally substituted with alkyl, cyano, halo, or haloalkyl; R³ is phenyl optionally substituted with alkyl, cyano, halo, haloalkyl, or haloalkoxy; R⁴ is alkyl, haloalkyl, or alkoxyalkylene; and R⁵ is hydrogen. In certain embodiments, R¹, R², R³, each independently, are optionally substituted phenyl; R⁵ is hydrogen; and R⁴ is alkyl. In certain embodiments, R¹, R², R³, each independently, are optionally substituted phenyl; R⁵ is hydrogen; and R⁴ is haloalkyl. In certain embodiments, R¹, R², R³, each independently, are optionally substituted phenyl; R⁵ is hydrogen; and R⁴ is alkoxyalkylene.

In certain embodiments, the compound or salt of Formula VII is that wherein: R¹ and R², each independently, are selected from phenyl optionally substituted with alkyl, cyano, halo, or haloalkyl; R³ is phenyl optionally substituted with alkyl, cyano, halo, haloalkyl, or haloalkoxy; and R⁴ and R⁵, together with the carbon atom to which they are attached, combine to form a C₃₋₆ spirocycloalkyl or a 3-6 membered spiroheterocycloalkyl, wherein the spirocycloalkyl is optionally substituted with 2 halo groups. In certain embodiments, R¹ and R², each independently, are selected from phenyl optionally substituted with alkyl, cyano, halo, or haloalkyl; R³ is phenyl optionally substituted with alkyl, cyano, halo, haloalkyl, or haloalkoxy; and R⁴ and R⁵, together with the carbon atom to which they are attached, combine to form a C₃₋₆ spirocycloalkyl that is substituted with 2 halo groups. In certain embodiments, R¹ and R², each independently, are selected from phenyl optionally substituted with alkyl, cyano, halo, or haloalkyl; R³ is phenyl optionally substituted with alkyl, cyano, halo, haloalkyl, or haloalkoxy; and R⁴ and R⁵, together with the carbon atom to which they are attached, combine to form a 3-6 membered spiroheterocycloalkyl.

In certain embodiments, the compound or salt of Formula VII is that wherein: R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl or a 5-6 membered heterocycloalkyl; and R⁵ is hydrogen, alkyl, haloalkyl, alkoxyalkylene or phenyl, wherein the phenyl is substituted with a group selected from cyano, haloalkyl, hydroxy, and haloalkoxy. In certain embodiments, R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl or a 5-6 membered heterocycloalkyl; and R⁵ is hydrogen or phenyl, wherein the phenyl is substituted with a group selected from cyano, haloalkyl, hydroxy, and haloalkoxy.

In certain embodiments, the compound or salt of Formula VII is that wherein: R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl or a 5-6 membered heterocycloalkyl; and R⁵ is hydrogen.

In certain embodiments, the compound or salt of Formula VII is that wherein: R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl or a 5-6 membered heterocycloalkyl; and R⁵ is phenyl, wherein the phenyl is substituted with a group selected from cyano, haloalkyl, hydroxy, and haloalkoxy. In certain embodiments, R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl or a 5-6 membered heterocycloalkyl; and R⁵ is phenyl, wherein the phenyl is substituted with a group selected from cyano, haloalkyl, hydroxy, and haloalkoxy, provided that R¹ and R² are same. In certain embodiments, R¹ and R², each independently, are selected from phenyl substituted with halo; R³ and R⁴, together with the carbon atoms to which they are attached, combine to form a C₅₋₆ cycloalkyl or a 5-6 membered heterocycloalkyl; and R⁵ is phenyl, wherein the phenyl is substituted with a group selected from cyano, haloalkyl, hydroxy, and haloalkoxy. In certain embodiments, R¹ and R², each independently, are selected from phenyl substituted with halo; R³ and R⁴, together with the carbon atoms to which they are attached, combine to form a C₅₋₆ cycloalkyl or a 5-6 membered heterocycloalkyl; and R⁵ is phenyl, wherein the phenyl is substituted with a group selected from cyano, haloalkyl, hydroxy, and haloalkoxy, provided that R¹ and R² are same.

In certain embodiments, the compound or salt of Formula VII is that wherein: R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl; and R⁵ is hydrogen or phenyl, wherein the phenyl is substituted with a group selected from cyano, haloalkyl, hydroxy, and haloalkoxy. In certain embodiments, R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl; and R⁵ is hydrogen. In certain embodiments, R¹ and R², each independently, are selected from phenyl optionally substituted with alkyl, cyano, halo, or haloalkyl; R³ and R⁴, together with the carbon atoms to which they are attached, combine to form a C₅₋₆ cycloalkyl; and R⁵ is phenyl substituted with a group selected from cyano, haloalkyl, hydroxy, and haloalkoxy.

In certain embodiments, the compound or salt of Formula VII is that wherein: R¹ and R², each independently, are selected from phenyl optionally substituted with alkyl, cyano, halo, or haloalkyl; R³ and R⁴, together with the carbon atoms to which they are attached, combine to form a 5-6 membered heterocycloalkyl; and R⁵ is hydrogen or phenyl, wherein the phenyl is substituted with a group selected from cyano, haloalkyl, hydroxy, and haloalkoxy. In certain embodiments, R¹ and R², each independently, are selected from phenyl optionally substituted with alkyl, cyano, halo, or haloalkyl; R³ and R⁴, together with the carbon atoms to which they are attached, combine to form a 5-6 membered heterocycloalkyl; and R⁵ is hydrogen. In certain embodiments, R¹ and R², each independently, are selected from phenyl optionally substituted with alkyl, cyano, halo, or haloalkyl; R³ and R⁴, together with the carbon atoms to which they are attached, combine to form a 5-6 membered heterocycloalkyl; and R⁵ is phenyl substituted with a group selected from cyano, haloalkyl, hydroxy, and haloalkoxy.

In certain embodiments, the compound or salt of Formula VII is that wherein: R¹ and R², each independently, are an optionally substituted aryl. In certain embodiments, R¹ and R², each independently, are substituted aryl. In certain embodiments, R¹ and R² are each an unsubstituted aryl. In certain embodiments, R¹ is substituted aryl and R² is an unsubstituted aryl. In certain embodiments, R¹ is an unsubstituted aryl and R² is substituted aryl.

In certain embodiments, the compound or salt of Formula VII is that wherein: R¹ and R², each independently, are aryl substituted with halo, cyano, nitro, alkyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino. In certain embodiments, R¹ and R², each independently, are aryl substituted with alkoxy, alkyl, cyano, halo, haloalkoxy, or haloalkyl. In certain embodiments, R¹ and R², each independently, are aryl substituted with alkoxy, alkyl, cyano, halo, or haloalkoxy. In certain embodiments, R¹ and R², each independently, are aryl substituted with alkoxy, alkyl, cyano, halo, or haloalkyl. In certain embodiments, R¹ and R², each independently, are aryl substituted with alkoxy, alkyl, cyano, haloalkoxy, or haloalkyl. In certain embodiments, R¹ and R², each independently, are aryl substituted with alkoxy, alkyl, halo, haloalkoxy, or haloalkyl. In certain embodiments, R¹ and R², each independently, are aryl substituted with alkoxy, cyano, halo, haloalkoxy, or haloalkyl. In certain embodiments, R¹ and R², each independently, are aryl substituted with alkyl, cyano, halo, haloalkoxy, or haloalkyl. In certain embodiments, R¹ and R², each independently, are aryl substituted with alkyl, alkenyl, cyano, halo, and haloalkyl. In certain embodiments, R¹ and R², each independently, are aryl substituted with alkyl, cyano, halo, or haloalkyl. In certain embodiments, R¹ and R², each independently, are aryl substituted with alkyl, cyano, or halo. In certain embodiments, R¹ and R², each independently, are aryl substituted with alkyl, cyano, or haloalkyl. In certain embodiments, R¹ and R², each independently, are aryl substituted with alkyl, halo, or haloalkyl. In certain embodiments, R¹ and R², each independently, are aryl substituted with cyano, halo, or haloalkyl. In certain embodiments, R¹ and R², each independently, are aryl substituted with alkyl or cyano. In certain embodiments, R¹ and R², each independently, are aryl substituted with alkyl or halo. In certain embodiments, R¹ and R², each independently, are aryl substituted with alkyl or haloalkyl. In certain embodiments, R¹ and R², each independently, are aryl substituted with a cyano or halo. In certain embodiments, R¹ and R², each independently, are aryl substituted with cyano or halolalkyl. In certain embodiments, R¹ and R², each independently, are aryl substituted with halo or haloalkyl. In certain embodiments, R¹ and R², each independently, are aryl substituted with alkoxy. In certain embodiments, R¹ and R², each independently, are aryl substituted with alkyl. In certain embodiments, R¹ and R², each independently, are aryl substituted with cyano. In certain embodiments, R¹ and R², each independently, are aryl substituted with halo. In certain embodiments, R¹ and R², each independently, are aryl substituted with haloalkoxy. In certain embodiments, R¹ and R², each independently, are aryl substituted with haloalkyl. In certain embodiments, R¹ and R² are same. In certain embodiments, R¹ and R² are different. In certain embodiments, R¹ and R², each independently, are substituted aryl, wherein the substituent is in ortho, meta, or para position on the aryl.

In certain embodiments, the compound or salt of Formula VII is that wherein R¹ and R², each independently, are optionally substituted with alkoxy, alkyl, cyano, halo, haloalkoxy, or haloalkyl. In certain embodiments, the compound or salt of Formula VII is that wherein R¹ and R², each independently, are optionally substituted with alkyl, cyano, halo, or haloalkyl.

In certain embodiments, the compound or salt of Formula VII is that wherein: R¹ and R², each independently, are an optionally substituted phenyl. In certain embodiments, R¹ and R², each independently, are substituted phenyl. In certain embodiments, R¹ and R² are each an unsubstituted phenyl. In certain embodiments, R¹ is substituted phenyl and R² is an unsubstituted phenyl. In certain embodiments, R¹ is an unsubstituted phenyl and R² is substituted phenyl.

In certain embodiments, the compound or salt of Formula VII is that wherein: R¹ and R², each independently, are phenyl substituted with halo, cyano, nitro, alkyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino. In certain embodiments, R¹ and R², each independently, are phenyl substituted with alkoxy, alkyl, cyano, halo, haloalkoxy, or haloalkyl. In certain embodiments, R¹ and R², each independently, are phenyl substituted with alkoxy, alkyl, cyano, halo, or haloalkoxy. In certain embodiments, R¹ and R², each independently, are phenyl substituted with alkoxy, alkyl, cyano, halo, or haloalkyl. In certain embodiments, R¹ and R², each independently, are phenyl substituted with alkoxy, alkyl, cyano, haloalkoxy, or haloalkyl. In certain embodiments, R¹ and R², each independently, are phenyl substituted with alkoxy, alkyl, halo, haloalkoxy, or haloalkyl. In certain embodiments, R¹ and R², each independently, are phenyl substituted with alkoxy, cyano, halo, haloalkoxy, or haloalkyl. In certain embodiments, R¹ and R², each independently, are phenyl substituted with alkyl, alkenyl, cyano, halo, and haloalkyl. In certain embodiments, R¹ and R², each independently, are phenyl substituted with alkyl, cyano, halo, haloalkoxy, or haloalkyl. In certain embodiments, R¹ and R², each independently, are phenyl substituted with alkyl, cyano, halo, or haloalkyl. In certain embodiments, R¹ and R², each independently, are phenyl substituted with alkyl, cyano, or halo. In certain embodiments, R¹ and R², each independently, are phenyl substituted with alkyl, cyano, or haloalkyl. In certain embodiments, R¹ and R², each independently, are phenyl substituted with alkyl, halo, or haloalkyl. In certain embodiments, R¹ and R², each independently, are phenyl substituted with cyano, halo, or haloalkyl. In certain embodiments, R¹ and R², each independently, are phenyl substituted with alkyl or cyano. In certain embodiments, R¹ and R², each independently, are phenyl substituted with methyl or cyano. In certain embodiments, R¹ and R², each independently, are phenyl substituted with alkyl or halo. In certain embodiments, R¹ and R², each independently, are phenyl substituted with methyl or chloro. In certain embodiments, R¹ and R², each independently, are phenyl substituted with alkyl or haloalkyl. In certain embodiments, R¹ and R², each independently, are phenyl substituted with a cyano or halo. In certain embodiments, R¹ and R², each independently, are phenyl substituted with a cyano, chloro, or bromo. In certain embodiments, R¹ and R², each independently, are phenyl substituted with cyano or halolalkyl. In certain embodiments, R¹ and R², each independently, are phenyl substituted with halo or haloalkyl. In certain embodiments, R¹ and R², each independently, are phenyl substituted with alkoxy. In certain embodiments, R¹ and R², each independently, are phenyl substituted with alkyl. In certain embodiments, R¹ and R², each independently, are phenyl substituted with cyano. In certain embodiments, R¹ and R², each independently, are phenyl substituted with halo. In certain embodiments, R¹ and R², each independently, are phenyl substituted with same halo group. In certain embodiments, R¹ and R², each independently, are phenyl substituted with chloro. In certain embodiments, R¹ and R², each independently, are phenyl substituted with haloalkoxy. In certain embodiments, R¹ and R², each independently, are phenyl substituted with haloalkyl. In certain embodiments, R¹ and R² are same. In certain embodiments, R¹ and R² are different. In certain embodiments, R¹ and R², each independently, are phenyl substituted with alkoxy, alkyl, cyano, halo, haloalkoxy, or haloalkyl, wherein the substituent is in ortho, meta, or para position on the phenyl. In certain embodiments, R¹ and R², each independently, are phenyl substituted with alkyl, cyano, halo, or haloalkyl, wherein the substituent is in ortho, meta, or para position on the phenyl. In certain embodiments, R¹ and R², each independently, are phenyl substituted with alkyl, cyano, halo, or haloalkyl, wherein the substituent is in meta position on the phenyl. In certain embodiments, R¹ and R², each independently, are phenyl substituted with alkyl, cyano, halo, or haloalkyl, wherein the substituent is in para position on the phenyl. In certain embodiments, R¹ and R², each independently, are phenyl substituted with alkyl, cyano, halo, or haloalkyl, wherein the substituent is in ortho position on the phenyl.

In certain embodiments, the compound or salt of Formula VII is that wherein: R¹ is phenyl substituted with halo, cyano, nitro, alkyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino. In certain embodiments, R¹ is phenyl substituted with alkoxy, alkyl, cyano, halo, haloalkoxy, or haloalkyl. In certain embodiments, R¹ is phenyl substituted with methoxy, ethoxy, methyl, ethyl, propyl, cyano, fluoro, chloro, bromo, iodo, trifluoromethoxy, difluoromethoxy, trifluoromethyl, or difluoromethyl. In certain embodiments, R¹ is phenyl substituted with alkyl, alkenyl, cyano, halo, and haloalkyl. In certain embodiments, R¹ is phenyl substituted with alkyl, cyano, halo, or haloalkyl. In certain embodiments, R¹ is phenyl substituted with methyl, ethyl, propyl, cyano, fluoro, chloro, bromo, iodo, trifluoromethyl, or difluoromethyl. In certain embodiments, R¹ is phenyl substituted with methyl, cyano, chloro, bromo, or trifluoromethyl. In certain embodiments, R¹ is phenyl substituted with alkyl, cyano, or halo. In certain embodiments, R¹ is phenyl substituted with alkyl, cyano, or haloalkyl. In certain embodiments, R¹ is phenyl substituted with methyl, cyano, or trifluoromethyl. In certain embodiments, R¹ is phenyl substituted with alkyl, halo, or haloalkyl. In certain embodiments, R¹ is phenyl substituted with methyl, chloro, bromo, or trifluoromethyl. In certain embodiments, R¹ is phenyl substituted with cyano, halo, or haloalkyl. In certain embodiments, R¹ is phenyl substituted with cyano, chloro, bromo, or trifluoromethyl. In certain embodiments, R¹ is phenyl substituted with alkyl or cyano. In certain embodiments, R¹ is phenyl substituted with methyl or cyano. In certain embodiments, R¹ is phenyl substituted with alkyl or halo. In certain embodiments, R¹ is phenyl substituted with methyl, chloro, or bromo. In certain embodiments, R¹ is phenyl substituted with alkyl or haloalkyl. In certain embodiments, R¹ is phenyl substituted with methyl or trifluoromethyl. In certain embodiments, R¹ is phenyl substituted with cyano or halo. In certain embodiments, R¹ is phenyl substituted with cyano, chloro, or bromo. In certain embodiments, R¹ is phenyl substituted with cyano or halolakyl. In certain embodiments, R¹ is phenyl substituted with cyano or trifluoromethyl. In certain embodiments, R¹ is phenyl substituted with halo or haloalkyl. In certain embodiments, R¹ is phenyl substituted with chloro, bromo, or trifluoromethyl. In certain embodiments, R¹ is phenyl substituted with alkoxy. In certain embodiments, R¹ is phenyl substituted with methoxy. In certain embodiments, R¹ is phenyl substituted with alkyl. In certain embodiments, R¹ is phenyl substituted with methyl. In certain embodiments, R¹ is phenyl substituted with cyano. In certain embodiments, R¹ is phenyl substituted with chloro or bromo. In certain embodiments, R¹ is phenyl substituted with chloro. In certain embodiments, R¹ is phenyl substituted with bromo. In certain embodiments, R¹ is phenyl substituted with haloalkoxy. In certain embodiments, R¹ is phenyl substituted with trifluoromethoxy. In certain embodiments, R¹ is phenyl substituted with trifluoromethyl. In certain embodiments, R¹ is phenyl substituted with alkoxy, alkyl, cyano, halo, haloalkoxy, or haloalkyl, wherein the substituent is in ortho, meta or, para position on the phenyl. In certain embodiments, R¹ is phenyl substituted with alkyl, cyano, halo, or haloalkyl, wherein the substituent is in ortho, meta or, para position on the phenyl. In certain embodiments, R¹ is phenyl substituted with alkyl, cyano, halo, or haloalkyl, wherein the substituent is in meta position on the phenyl. In certain embodiments, R¹ is phenyl substituted with alkyl, cyano, halo, or haloalkyl, wherein the substituent is in para position on the phenyl. In certain embodiments, R¹ is phenyl substituted with alkyl, cyano, halo, or haloalkyl, wherein the substituent is in ortho position on the phenyl.

In certain embodiments, the compound or salt of Formula VII is that wherein R¹ is an unsubstituted phenyl.

In certain embodiments, the compound or salt of Formula VII is that wherein: R² is phenyl substituted with halo, cyano, nitro, alkyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino. In certain embodiments, R² is phenyl substituted with alkoxy, alkyl, cyano, halo, haloalkoxy, or haloalkyl. In certain embodiments, R² is phenyl substituted with methoxy, ethoxy, methyl, ethyl, propyl, cyano, fluoro, chloro, bromo, iodo, trifluoromethoxy, difluoromethoxy, trifluoromethyl, or difluoromethyl. In certain embodiments, R² is phenyl substituted with alkyl, alkenyl, cyano, halo, and haloalkyl. In certain embodiments, R² is phenyl substituted with alkyl, cyano, halo, or haloalkyl. In certain embodiments, R² is phenyl substituted with methyl, ethyl, propyl, cyano, fluoro, chloro, bromo, iodo, trifluoromethyl, or difluoromethyl. In certain embodiments, R² is phenyl substituted with methyl, cyano, chloro, bromo, or trifluoromethyl. In certain embodiments, R² is phenyl substituted with alkyl, cyano, or halo. In certain embodiments, R² is phenyl substituted with alkyl, cyano, or haloalkyl. In certain embodiments, R² is phenyl substituted with methyl, cyano, or trifluoromethyl. In certain embodiments, R² is phenyl substituted with alkyl, halo, or haloalkyl. In certain embodiments, R² is phenyl substituted with methyl, chloro, bromo, or trifluoromethyl. In certain embodiments, R² is phenyl substituted with cyano, halo, or haloalkyl. In certain embodiments, R² is phenyl substituted with cyano, chloro, bromo, or trifluoromethyl. In certain embodiments, R² is phenyl substituted with alkyl or cyano. In certain embodiments, R² is phenyl substituted with methyl or cyano. In certain embodiments, R² is phenyl substituted with alkyl or halo. In certain embodiments, R² is phenyl substituted with methyl, chloro, or bromo. In certain embodiments, R² is phenyl substituted with alkyl or haloalkyl. In certain embodiments, R² is phenyl substituted with methyl or trifluoromethyl. In certain embodiments, R² is phenyl substituted with cyano or halo. In certain embodiments, R² is phenyl substituted with cyano, chloro, or bromo. In certain embodiments, R² is phenyl substituted with cyano or halolakyl. In certain embodiments, R² is phenyl substituted with cyano or trifluoromethyl. In certain embodiments, R² is phenyl substituted with halo or haloalkyl. In certain embodiments, R² is phenyl substituted with chloro, bromo, or trifluoromethyl. In certain embodiments, R² is phenyl substituted with alkoxy. In certain embodiments, R² is phenyl substituted with methoxy. In certain embodiments, R² is phenyl substituted with alkyl. In certain embodiments, R² is phenyl substituted with methyl. In certain embodiments, R² is phenyl substituted with cyano. In certain embodiments, R² is phenyl substituted with chloro or bromo. In certain embodiments, R² is phenyl substituted with chloro. In certain embodiments, R² is phenyl substituted with bromo. In certain embodiments, R² is phenyl substituted with haloalkoxy. In certain embodiments, R² is phenyl substituted with trifluoromethoxy. In certain embodiments, R² is phenyl substituted with trifluoromethyl. In certain embodiments, R² is phenyl substituted with alkoxy, alkyl, cyano, halo, haloalkoxy, or haloalkyl, wherein the substituent is in ortho, meta or, para position on the phenyl. In certain embodiments, R² is phenyl substituted with alkyl, cyano, halo, or haloalkyl, wherein the substituent is in ortho, meta, or para position on the phenyl. In certain embodiments, R² is phenyl substituted with alkyl, cyano, halo, or haloalkyl, wherein the substituent is in meta position on the phenyl. In certain embodiments, R² is phenyl substituted with alkyl, cyano, halo, or haloalkyl, wherein the substituent is in para position on the phenyl. In certain embodiments, R² is phenyl substituted with alkyl, cyano, halo, or haloalkyl, wherein the substituent is in ortho position on the phenyl.

In certain embodiments, the compound or salt of Formula VII is that wherein R² is an unsubstituted phenyl.

In certain embodiments, the compound or salt of Formula VII is that wherein R³ is an optionally substituted aryl. In certain embodiments, R³ is substituted aryl. In certain embodiments, R³ is an unsubstituted aryl.

In certain embodiments, the compound or salt of Formula VII is that wherein R³ is aryl substituted with halo, cyano, nitro, hydroxy, alkyl, alkenyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl. In certain embodiments, R³ is aryl substituted with alkyl, alkenyl, cyano, halo, haloalkyl, alkoxy, and haloalkoxy. In certain embodiments, the compound or salt of Formula VII is that wherein R³ is aryl substituted with alkyl, cyano, halo, haloalkyl, or haloalkoxy. In certain embodiments, R³ is aryl substituted with alkyl, halo, haloalkyl, or haloalkoxy. In certain embodiments, R³ is aryl substituted with alkyl, cyano, haloalkyl, or haloalkoxy. In certain embodiments, R³ is aryl substituted with alkyl, cyano, halo, or haloalkoxy. In certain embodiments, R³ is aryl substituted with alkyl, cyano, halo, or haloalkyl. In certain embodiments, R³ is aryl substituted with cyano, halo, haloalkyl, or haloalkoxy. In certain embodiments, R³ is aryl substituted with alkyl, cyano, or halo. In certain embodiments, R³ is aryl substituted with haloalkyl, or haloalkoxy. In certain embodiments, R³ is aryl substituted with cyano or haloalkoxy. In certain embodiments, R³ is aryl substituted with alkyl or halo. In certain embodiments, R³ is aryl substituted with alkyl or haloalkyl. In certain embodiments, R³ is aryl substituted with alkyl or cyano. In certain embodiments, R³ is aryl substituted with alkyl or haloalkoxy. In certain embodiments, R³ is aryl substituted with alkyl. In certain embodiments, R³ is aryl substituted with cyano. In certain embodiments, R³ is aryl substituted with halo. In certain embodiments, R³ is aryl substituted with haloalkyl. In certain embodiments, R³ is aryl substituted with haloalkoxy.

In certain embodiments, the compound or salt of Formula VII is that wherein R³ is an optionally substituted phenyl. In certain embodiments, R³ is substituted phenyl. In certain embodiments, R³ is an unsubstituted phenyl.

In certain embodiments, the compound or salt of Formula VII is that wherein R³ is phenyl substituted with halo, cyano, nitro, hydroxy, alkyl, alkenyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl. In certain embodiments, R³ is phenyl substituted with alkyl, alkenyl, cyano, halo, haloalkyl, alkoxy, and haloalkoxy. In certain embodiments, the compound or salt of Formula VII is that wherein R³ is phenyl substituted with alkyl, cyano, halo, haloalkyl, or haloalkoxy. In certain embodiments, R³ is phenyl substituted with fluoro, chloro, bromo, iodo, methyl, ethyl, propyl, trifluoromethyl, difluoromethyl, cyano, trifluoromethoxy, or difluoromethoxy. In certain embodiments, R³ is phenyl substituted with chloro, methyl, trifluoromethyl, cyano, trifluoromethoxy, or difluoromethoxy. In certain embodiments, R³ is phenyl substituted with alkyl, halo, haloalkyl, or haloalkoxy. In certain embodiments, R³ is phenyl substituted with alkyl, cyano, haloalkyl, or haloalkoxy. In certain embodiments, R³ is phenyl substituted with alkyl, cyano, halo, or haloalkoxy. In certain embodiments, R³ is phenyl substituted with alkyl, cyano, halo, or haloalkyl. In certain embodiments, R³ is phenyl substituted with cyano, halo, haloalkyl, or haloalkoxy. In certain embodiments, R³ is phenyl substituted with alkyl, cyano, or halo. In certain embodiments, R³ is phenyl substituted with haloalkyl, or haloalkoxy. In certain embodiments, R³ is phenyl substituted with cyano or haloalkoxy. In certain embodiments, R³ is phenyl substituted with alkyl or halo. In certain embodiments, R³ is phenyl substituted with alkyl or haloalkyl. In certain embodiments, R³ is phenyl substituted with alkyl or cyano. In certain embodiments, R³ is phenyl substituted with alkyl or haloalkoxy. In certain embodiments, R³ is phenyl substituted with alkyl. In certain embodiments, R³ is phenyl substituted with methyl. In certain embodiments, R³ is phenyl substituted with cyano. In certain embodiments, R³ is phenyl substituted with halo. In certain embodiments, R³ is phenyl substituted with chloro. In certain embodiments, R³ is phenyl substituted with fluoro, chloro, or bromo. In certain embodiments, R³ is phenyl substituted with haloalkyl. In certain embodiments, R³ is phenyl substituted with trifluoromethyl. In certain embodiments, R³ is phenyl substituted with trifluoromethoxy, or difluoromethoxy. In certain embodiments, R³ is phenyl substituted with trifluoromethoxy. In certain embodiments, R³ is phenyl substituted with difluoromethoxy. In certain embodiments, R³ is phenyl substituted with alkyl, cyano, halo, haloalkyl, or haloalkoxy, wherein the substituent is in ortho, meta, or para position on the phenyl. In certain embodiments, R³ is phenyl substituted with alkyl, cyano, halo, haloalkyl, or haloalkoxy, wherein the substituent is in ortho, position on the phenyl. In certain embodiments, R³ is phenyl substituted with alkyl, cyano, halo, haloalkyl, or haloalkoxy, wherein the substituent is in meta position on the phenyl. In certain embodiments, R³ is phenyl substituted with alkyl, cyano, halo, haloalkyl, or haloalkoxy, wherein the substituent is in para position on the phenyl.

In certain embodiments, the compound or salt of Formula VII is that wherein R³ is optionally substituted with alkyl, cyano, halo, haloalkyl, or haloalkoxy.

In certain embodiments, the compound or salt of Formula VII is that wherein R³ and R⁴, together with the carbon atoms to which they are attached combine to form a C₅₋₆ cycloalkyl or a 5-6 membered heterocycloalkyl. In certain embodiments, R³ and R⁴, together with the carbon atoms to which they are attached combine to form C₅₋₆ cycloalkyl. In certain embodiments, R³ and R⁴, together with the carbon atoms to which they are attached combine to form C₅₋₆ cycloalkyl, wherein the cycloalkyl is cyclohexyl or cyclopentyl. In certain embodiments, R³ and R⁴, together with the carbon atoms to which they are attached combine to form C₆ cycloalkyl. In certain embodiments, R³ and R⁴, together with the carbon atoms to which they are attached combine to form cyclohexyl. In certain embodiments, R³ and R⁴, together with the carbon atoms to which they are attached combine to form C₅ cycloalkyl. In certain embodiments, R³ and R⁴, together with the carbon atoms to which they are attached combine to form cyclopentyl. In certain embodiments, R³ and R⁴, together with the carbon atoms to which they are attached combine to form a 5-6 membered heterocycloalkyl. In certain embodiments, R³ and R⁴, together with the carbon atoms to which they are attached combine to form 6 membered heterocycloalkyl. In certain embodiments, R³ and R⁴, together with the carbon atoms to which they are attached combine to form

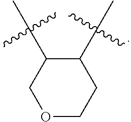 [SMILES: *C1CCOCC1*]

In certain embodiments, the compound or salt of Formula VII is that wherein R⁴ is hydrogen, alkyl, haloalkyl, or alkoxyalkylene. In certain embodiments, R⁴ is hydrogen, methyl, ethyl, propyl, trifluoromethyl, or methoxymethylene. In certain embodiments, R⁴ is alkyl, haloalkyl, or alkoxyalkylene. In certain embodiments, R⁴ is methyl, ethyl, propyl, trifluoromethyl, or methoxymethylene. In certain embodiments, R⁴ is hydrogen, alkyl or haloalkyl. In certain embodiments, the compound or salt of Formula VII is that wherein R⁴ is hydrogen, methyl, ethyl, propyl, or trifluoromethyl. In certain embodiments, R⁴ is alkyl or alkoxyalkylene. In certain embodiments, the compound or salt of Formula VII is that wherein R⁴ is methyl, ethyl, propyl, or methoxymethylene. In certain embodiments, R⁴ is haloalkyl or alkoxyalkylene. In certain embodiments, R⁴ is trifluoromethyl, or methoxymethylene. In certain embodiments, R⁴ is alkyl. In certain embodiments, R⁴ is methyl, ethyl, or propyl. In certain embodiments, R⁴ is methyl. In certain embodiments, R⁴ is ethyl. In certain embodiments, R⁴ is propyl. In certain embodiments, R⁴ is haloalkyl. In certain embodiments, R⁴ is trifluoromethyl. In certain embodiments, R⁴ is methoxymethylene.

In certain embodiments, the compound or salt of Formula VII is that wherein R⁴ is hydrogen.

In certain embodiments, the compound or salt of Formula VII is that wherein R⁵ is hydrogen, alkyl, haloalkyl, alkoxyalkylene, cycloalkyl, or aryl, wherein the aryl is optionally substituted with a group selected from alkyl, cyano, haloalkyl, hydroxy, alkoxy, and haloalkoxy. In certain embodiments, R⁵ is hydrogen, alkyl, alkenyl, haloalkyl, or alkoxyalkylene, or aryl, wherein the aryl is optionally substituted with a group selected from alkyl, cyano, haloalkyl, hydroxy, alkoxy, and haloalkoxy. In certain embodiments, R⁵ is hydrogen, alkyl, haloalkyl, or alkoxyalkylene. In certain embodiments, R⁵ is hydrogen, methyl, ethyl, propyl, trifluoromethyl, or methoxymethylene. In certain embodiments, R⁵ is alkyl, haloalkyl, or alkoxyalkylene. In certain embodiments, R⁵ is methyl, ethyl, propyl, trifluoromethyl, or methoxymethylene. In certain embodiments, R⁵ is hydrogen, alkyl or haloalkyl. In certain embodiments, the compound or salt of Formula VII is that wherein R⁵ is hydrogen, methyl, ethyl, propyl, or trifluoromethyl. In certain embodiments, R⁵ is alkyl or alkoxyalkylene. In certain embodiments, the compound or salt of Formula VII is that wherein R⁵ is methyl, ethyl, propyl, or methoxymethylene. In certain embodiments, R⁵ is haloalkyl or alkoxyalkylene. In certain embodiments, R⁵ is trifluoromethyl, or methoxymethylene. In certain embodiments, R⁵ is alkyl. In certain embodiments, R⁵ is methyl, ethyl, or propyl. In certain embodiments, R⁵ is methyl. In certain embodiments, R⁵ is ethyl. In certain embodiments, R⁵ is propyl. In certain embodiments, R⁵ is haloalkyl. In certain embodiments, R⁵ is trifluoromethyl. In certain embodiments, R⁵ is methoxymethylene.

In certain embodiments, when R¹ and R² are both 4-methoxyphenyl, neither R⁴ nor R⁵ is tert-butyl. In certain embodiments, when R¹ and R² are both 4-methoxyphenyl, R⁴ is not tert-butyl. In certain embodiments, when R¹ and R² are both 4-methoxyphenyl, R⁵ is not tert-butyl. In certain embodiments, when R¹ and R² are both 4-methoxyphenyl, R⁴ is not tert-butyl, and R⁵ is not hydrogen. In certain embodiments, when R¹ and R² are both 4-methoxyphenyl, R⁴ is not hydrogen, and R⁵ is not tert-butyl. In certain embodiments, when R¹ and R² are both 4-methoxyphenyl, R⁴ is hydrogen, and R⁵ is tert-butyl.

In certain embodiments, the compound of Formula VII is not (4S,5S)-4-(tert-butyl)-1,3-bis(4-methoxyphenyl)-5-phenylimidazolidin-2-one. In certain embodiments, the compound of Formula VII is not 4-(tert-butyl)-1,3-bis(4-methoxyphenyl)-5-phenylimidazolidin-2-one.

In certain embodiments, the compound of Formula VII is that wherein when R¹ and R², are each independently unsubstituted phenyl or phenyl substituted with methyl, methoxy, or chloro; and one of R³ and R⁵ is unsubstituted phenyl or phenyl substituted with methyl, methoxy, chloro, or trifluoromethyl; then the other of R³ and R⁵ is not unsubstituted phenyl or phenyl substituted with methyl, methoxy, chloro, or trifluoromethyl. In certain embodiments, the compound of Formula VII is that wherein R¹ and R² are not substituted with alkoxy.

In certain embodiments, the compound of Formula VII is that wherein R³ and R⁴, together with the carbon atoms to which they are attached combine to form a 5-6 membered heterocycloalkyl containing only O and N ring heteroatom. In certain embodiments, the compound of Formula VII is that wherein R³ and R⁴, together with the carbon atoms to which they are attached combine to form a 5-6 membered heterocycloalkyl containing only an O ring heteroatom. In certain embodiments, the compound of Formula VII is that wherein R³ and R⁴, together with the carbon atoms to which they are attached combine to form a 5-6 membered heterocycloalkyl containing only N ring heteroatom.

In certain embodiments, the compound of Formula VII is that wherein R³ and R⁴, together with the carbon atoms to which they are attached combine to form a saturated C₅. 6 cycloalkyl.

In certain embodiments, the compound of Formula VII is that wherein when R³ and R⁴, together with the carbon atoms to which they are attached combine to form a 6 membered heterocycloalkyl containing only an O ring heteroatom, then R¹ and R² are both mono-substituted aryl.

In certain embodiments, the compound of Formula VII is that wherein when R³ and R⁴, together with the carbon atoms to which they are attached combine to form a saturated cycloalkyl or a saturated heterocycloalkyl containing only an O ring heteroatom, then R¹ and R² are both mono-substituted aryl.

In certain embodiments, the compound or salt of Formula VII is that wherein R⁵ is hydrogen.

In certain embodiments, the compound or salt of Formula VII is that wherein R⁵ is aryl, wherein the aryl is substituted with a group selected from cyano, haloalkyl, hydroxy, and haloalkoxy. In certain embodiments, R⁵ is phenyl, wherein the phenyl is substituted with a group selected from cyano, haloalkyl, hydroxy, and haloalkoxy. In certain embodiments, R⁵ is phenyl, wherein the phenyl is substituted with a group selected from cyano, difluoromethyl, trifluoromethyl, hydroxy, trifluoromethoxy, and difluoromethoxy. In certain embodiments, R⁵ is phenyl, wherein the phenyl is substituted with a group selected from cyano, haloalkyl, and haloalkoxy. In certain embodiments, R⁵ is phenyl, wherein the phenyl is substituted with a group selected from cyano, difluoromethyl, trifluoromethyl, trifluoromethoxy, and difluoromethoxy. In certain embodiments, R⁵ is phenyl, wherein the phenyl is substituted with a group selected from cyano, haloalkyl, and hydroxy. In certain embodiments, R⁵ is phenyl, wherein the phenyl is substituted with a group selected from cyano, difluoromethyl, trifluoromethyl, and hydroxy. In certain embodiments, R⁵ is phenyl, wherein the phenyl is substituted with a group selected from cyano and haloalkyl. In certain embodiments, R⁵ is phenyl, wherein the phenyl is substituted with a group selected from cyano, difluoromethyl, and trifluoromethyl. In certain embodiments, R⁵ is phenyl, wherein the phenyl is substituted with a group selected from cyano and hydroxy. In certain embodiments, R⁵ is phenyl, wherein the phenyl is substituted with a group selected from cyano and hydroxy. In certain embodiments, R⁵ is phenyl, wherein the phenyl is substituted with a group selected from cyano and haloalkoxy. In certain embodiments, R⁵ is phenyl, wherein the phenyl is substituted with a group selected from cyano, trifluoromethoxy, and difluoromethoxy. In certain embodiments, R⁵ is phenyl, wherein the phenyl is substituted with a group selected from haloalkyl and haloalkoxy. In certain embodiments, R⁵ is phenyl, wherein the phenyl is substituted with a group selected from difluoromethyl, trifluoromethyl, trifluoromethoxy, and difluoromethoxy. In certain embodiments, R⁵ is phenyl substituted with a cyano. In certain embodiments, R⁵ is phenyl substituted with haloalkyl. In certain embodiments, R⁵ is phenyl substituted with a group selected from difluoromethyl and trifluoromethyl. In certain embodiments, R⁵ is phenyl substituted with hydroxy. In certain embodiments, R⁵ is phenyl substituted with haloalkoxy. In certain embodiments, R⁵ is phenyl substituted with a group selected from trifluoromethoxy, and difluoromethoxy.

In certain embodiments, the compound or salt of Formula VII is that wherein R¹ and R², each independently, are selected from phenyl substituted with alkyl, cyano, halo, or haloalkyl; and R⁵ is phenyl, wherein the phenyl is substituted with a group selected from cyano, haloalkyl, hydroxy, and haloalkoxy. In certain embodiments, R¹ and R², each independently, are selected from phenyl substituted with alkyl, cyano, halo, or haloalkyl; and R⁵ is phenyl, wherein the phenyl is substituted with a group selected from cyano, haloalkyl, hydroxy, and haloalkoxy, provided that R¹ and R² are same. In certain embodiments, R¹ and R², each independently, are selected from phenyl substituted with halo; and R⁵ is phenyl, wherein the phenyl is substituted with a group selected from cyano, haloalkyl, hydroxy, and haloalkoxy, provided that R¹ and R² are same. In certain embodiments, R¹ and R², each independently, are selected from phenyl substituted with chloro or bromo; and R⁵ is phenyl, wherein the phenyl is substituted with a group selected from cyano, haloalkyl, hydroxy, and haloalkoxy, provided that R¹ and R² are same.

In certain embodiments, the compound or salt of Formula VII is that wherein R⁴ and R⁵, each independently, are selected from hydrogen, alkyl, haloalkyl, and alkoxyalkylene; and provided that R⁴ and R⁵ are not both hydrogen. In certain embodiments, R⁴ and R⁵, each independently, are selected from hydrogen, methyl, ethyl, propyl, butyl, trifluoromethyl, and methoxymethylene; and provided that R⁴ and R⁵ are not both hydrogen. In certain embodiments, R⁴ and R⁵, each independently, are selected from hydrogen, methyl, ethyl, propyl, trifluoromethyl, and methoxymethylene; and provided that R⁴ and R⁵ are not both hydrogen. In certain embodiments, R⁴ and R⁵, each independently, are selected from alkyl, haloalkyl, and alkoxyalkylene. In certain embodiments, R⁴ and R⁵, each independently, are selected from methyl, ethyl, propyl, trifluoromethyl, and methoxymethylene. In certain embodiments, R⁴ and R⁵, each independently, are selected from hydrogen and alkyl; and provided that R⁴ and R⁵ are not both hydrogen. In certain embodiments, R⁴ and R⁵, each independently, are selected from hydrogen, methyl, ethyl, and propyl; and provided that R⁴ and R⁵ are not both hydrogen. In certain embodiments, R⁴ and R⁵, each independently, are selected from hydrogen and methyl; and provided that R⁴ and R⁵ are not both hydrogen. In certain embodiments, R⁴ and R⁵, each independently, are selected from alkyl and haloalkyl. In certain embodiments, R⁴ and R⁵, each independently, are selected from methyl, ethyl, propyl, and trifluoromethyl. In certain embodiments, R⁴ and R⁵, each independently, are selected from methyl and trifluoromethyl. In certain embodiments, R⁴ and R⁵, each independently, are selected from alkyl and haloalkoxy. In certain embodiments, R⁴ and R⁵, each independently, are selected from methyl, ethyl, propyl, and methoxymethylene. In certain embodiments, R⁴ and R⁵, each independently, are selected from methyl, and methoxymethylene. In certain embodiments, R⁴ and R⁵, each independently, are selected from alkyl. In certain embodiments, R⁴ and R⁵, each independently, are selected from methyl, ethyl and propyl. In certain embodiments, R⁴ and R⁵, each independently, are methyl. In certain embodiments, R⁴ and R⁵, each independently, are ethyl. In certain embodiments, R⁴ and R⁵, each independently, are selected from methyl and ethyl. In certain embodiments, R⁴ and R⁵, each independently, are selected from methyl and propyl.

In certain embodiments, R⁴ is hydrogen, alkyl, haloalkyl, or alkoxyalkylene; and R⁵ is alkyl, haloalkyl, or alkoxyalkylene. In certain embodiments, R⁴ is hydrogen; and R⁵ is alkyl, haloalkyl, or alkoxyalkylene. In certain embodiments, R⁴ is hydrogen; and R⁵ is methyl, ethyl, propyl, trifluoromethyl, or methoxymethylene. In certain embodiments, R⁴ is hydrogen; and R⁵ is alkyl. In certain embodiments, R⁴ is hydrogen; and R⁵ is haloalkyl. In certain embodiments, R⁴ is hydrogen; and R⁵ is alkoxyalkylene.

In certain embodiments, R⁴ is alkyl, haloalkyl, or alkoxyalkylene; and R⁵ is hydrogen, alkyl, haloalkyl, or alkoxyalkylene. In certain embodiments, R⁴ is alkyl, haloalkyl, or alkoxyalkylene; and R⁵ is hydrogen. In certain embodiments, R⁴ is methyl, ethyl, propyl, butyl, trifluoromethyl, or methoxymethylene; and R⁵ is hydrogen. In certain embodiments, R⁴ is alkyl, and R⁵ is hydrogen. In certain embodiments, R⁴ is haloalkyl, and R⁵ is hydrogen. In certain embodiments, R⁴ is alkoxyalkylene, and R⁵ is hydrogen.

In certain embodiments, the compound or salt of Formula VII is that wherein R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 halo group(s). In certain embodiments, the compound or salt of Formula VII is that wherein R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups. In certain embodiments, R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 halo group. In certain embodiments, R⁴ and R⁵, together with the carbon atom to which they are attached combine to form a C₃₋₆ spirocycloalkyl or a 3-6 membered spiroheterocycloalkyl, wherein the spirocycloalkyl is substituted with 2 halo groups. In certain embodiments, R⁴ and R⁵, together with the carbon atom to which they are attached combine to form a C₃₋₆ spirocycloalkyl or a 3-6 membered spiroheterocycloalkyl. In certain embodiments, R⁴ and R⁵, together with the carbon atom to which they are attached combine to form a C₃₋₆ spirocycloalkyl, wherein the spirocycloalkyl is optionally substituted with 2 halo groups. In certain embodiments, R⁴ and R⁵, together with the carbon atom to which they are attached combine to form C₃₋₆ spirocycloalkyl, wherein the spirocycloalkyl is spirocyclopentyl or spirocyclobutyl, wherein the spirocyclobutyl is substituted with 2 fluoro groups. In certain embodiments, R⁴ and R⁵, together with the carbon atom to which they are attached combine to form unsubstituted C₃₋₆ spirocycloalkyl. In certain embodiments, R⁴ and R⁵, together with the carbon atom to which they are attached combine to form a C₃₋₆ spirocycloalkyl substituted with 2 halo groups. In certain embodiments, R⁴ and R⁵, together with the carbon atom to which they are attached combine to form 3-6 membered spiroheterocycloalkyl.

In certain embodiments, the compound or salt of Formula VII is that wherein R⁴ and R⁵, together with the carbon atom to which they are attached combine to form a C₄ spirocycloalkyl substituted with 2 halo groups. In certain embodiments, R⁴ and R⁵, together with the carbon atom to which they are attached combine to form a spirocyclobutyl substituted with 2 fluoro groups. In certain embodiments, R⁴ and R⁵, together with the carbon atom to which they are attached combine to form a C₄ spirocycloalkyl. In certain embodiments, R⁴ and R⁵, together with the carbon atom to which they are attached combine to form a spirocyclobutyl. In certain embodiments, R⁴ and R⁵, together with the carbon atom to which they are attached combine to form a C₅ spirocycloalkyl. In certain embodiments, R⁴ and R⁵, together with the carbon atom to which they are attached combine to form a spirocyclopentyl.

In certain embodiments, the compound or salt of Formula VII is that wherein R⁴ and R⁵, together with the carbon atom to which they are attached combine to form a 4 membered spiroheterocycloalkyl. In certain embodiments, R⁴ and R⁵, together with the carbon atom to which they are attached combine to form

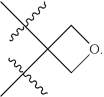 [SMILES: *C1(*)COC1]

In certain embodiments, the compound or salt of Formula VII is that according to one of the following formulas:

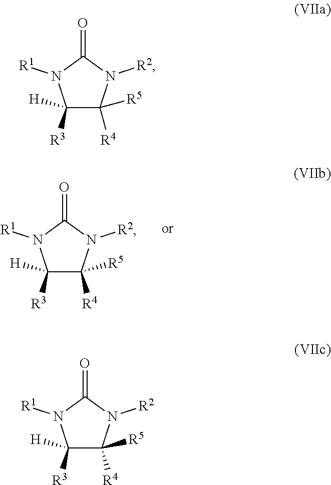

where all groups are as defined in the Summary or as defined and/substituted in any one of the embodiments described herein.

In certain embodiments, the compound or salt of Formula VII is that according to one of the following formulas:

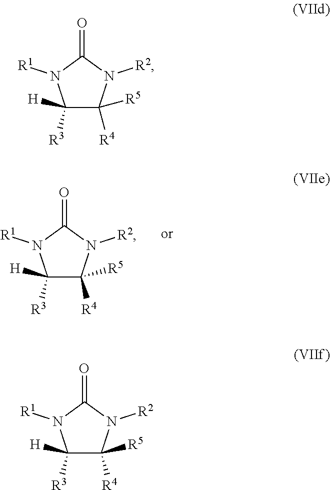

where all groups are as defined in the Summary or as defined and/substituted in any one of the embodiments described herein.

In certain embodiments, the compound or salt of Formula VII is that according to one of the following formulas:

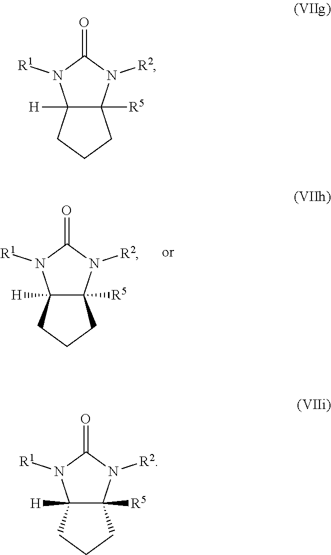

In certain embodiments, the compound or salt of Formula VIIg, VIIh, or VIi is that wherein R¹ and R² are phenyl substituted with halo; and R⁵ is substituted phenyl. In certain embodiments, the compound or salt of Formula VIIg, VIIh, or VIi is that wherein R¹ and R² are phenyl substituted with chloro; and R⁵ is substituted phenyl.

In certain embodiments, the compound or salt of Formula VII is that according to one of the following formulas:

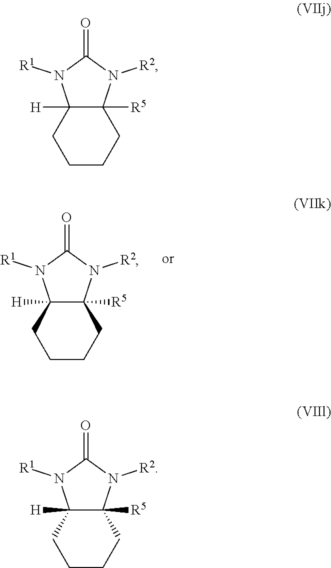

In certain embodiments, the compound or salt of Formula VIIj, VIIk, or VIIl is that wherein R¹ and R² are phenyl substituted with halo; and R⁵ is substituted phenyl. In certain embodiments, the compound or salt of Formula VIIj, VIIk, or VIIl is that wherein R¹ and R² are phenyl substituted with chloro; and R⁵ is substituted phenyl.

In certain embodiments, the compound or salt of Formula VII is that according to one of the following formulas:

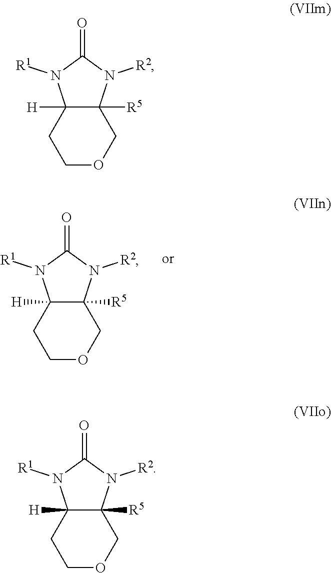

In certain embodiments, the compound or salt of Formula VIIm, VIIn, or VIIo is that wherein R¹ and R² are phenyl substituted with halo; and R⁵ is substituted phenyl. In certain embodiments, the compound or salt of Formula VIIm, VIIn, or VIIo is that wherein R¹ and R² are phenyl substituted with chloro; and R⁵ is substituted phenyl.

In certain embodiments, the compound or salt of Formula VII is that according to one of the following formulas:

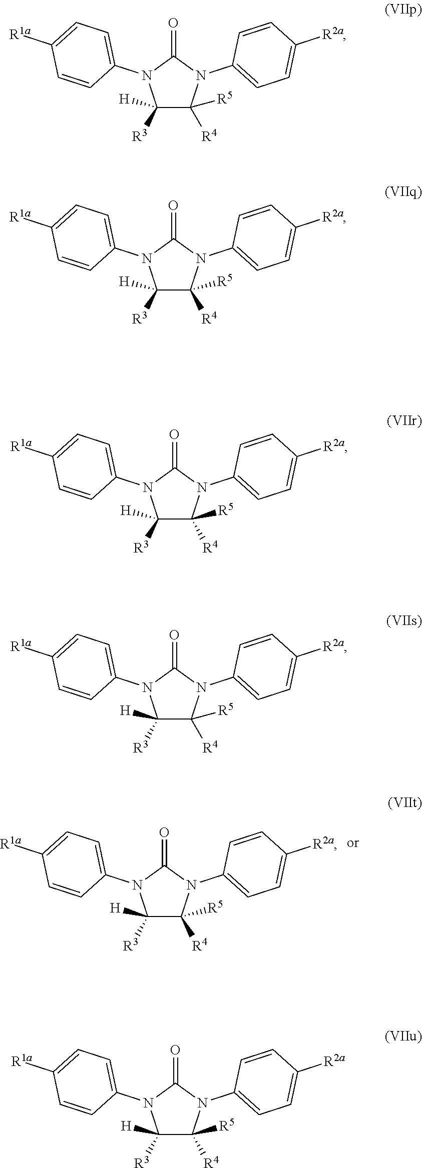

wherein R^{1a} and R^{2a}, each independently, are alkyl, cyano, halo or haloalkyl, wherein all groups are as defined in the Summary or as defined and/substituted in any one of the embodiments described herein.

In certain embodiments, the compound or salt of Formula VII is that according to one of the following formulas:

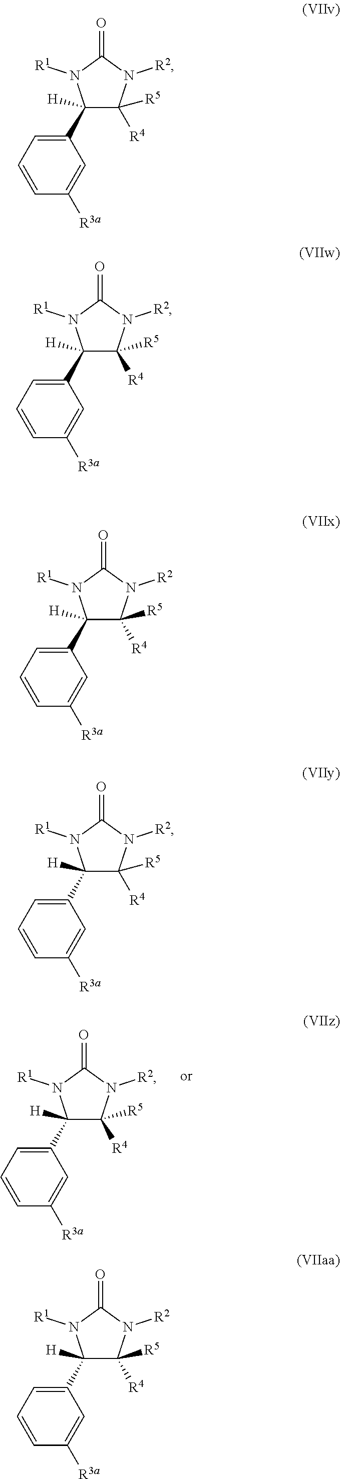

wherein R^{3a} is alkyl, cyano, halo or haloalkyl, wherein all groups are as defined in the Summary or as defined and/substituted in any one of the embodiments described herein.

In certain embodiments, the compound or salt of Formula VII is that according to one of the following formulas:

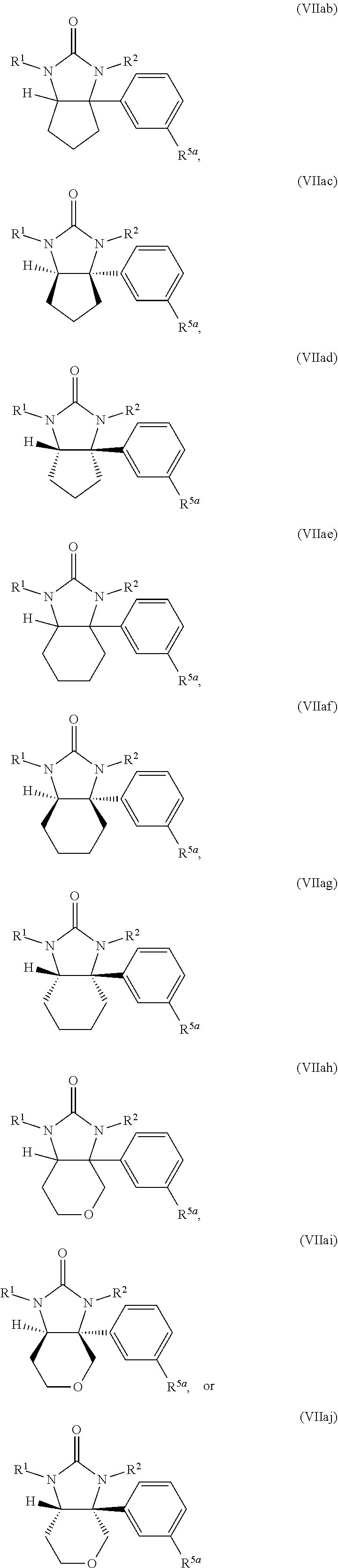

wherein R^{5a} is alkyl, cyano, halo or haloalkyl, wherein all groups are as defined in the Summary or as defined and/substituted in any one of the embodiments described herein.

In certain embodiments, the compound or salt thereof is a compound of Formula VII, VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, VIIaf, VIIag, VIIah, VIIai, or VIIaj, or a single stereoisomer or mixture of stereoisomers thereof, as defined herein.

In certain embodiments, the compound or salt thereof is selected from Table 2. In certain embodiments, the compound or salt thereof is selected from the group consisting of the compounds of Table 2 consisting of Examples 2-2, 6, 6-2, 7, 7-2, 8, 8-2, 9, 9-2, 10, 10-2, 11-2, 14, 15-2, 17, 17-1, 19-2, 19-3, 20-1, 21-2, 22-2, 22-3, 24-1, 25-1, 27-2, 28-2, 29-2, 30-2, 31-2, 32-2, 33-1, 33-4, 34-1, 34-3, 35-2, 36-2, 37-2, 38-2, 39-1, 40-2, 41-2, 42-1, 43-2, 44-2, 45-2, 46-1, 47-2, 48-2, 49-2, 50-1, 51-2, 52-2, 53-2, 54-2, 52-5, 56-2, and 57-2, or a single stereoisomer or mixture of stereoisomers thereof.

In certain embodiments, the compound or salt thereof is selected from the group consisting of the compounds of Table 2 consisting of Examples 1, 2-3, 2-1, 2-2, 3, 4-1, 4-2, 5-1, 5-2, 6, 7, 8, 9, 10, 8-1, 8-2, 10-1, 10-2, 7-1, 7-2, 6-1, 6-2, 9-1, 9-2, 11-1, 11-2, 12, 12-1, 12-2, 13-1, 13-2, 14, 15-1, 16-1, 16-2, 17, 18, 15-2, 15-1, 19-1, 19-2, 19-3, 19-4, 20-1, 20-2, 21-1, 21-2, 22-2, 22-3, 22-5, 23-1, 23-2, 24-1. 24-2, 25-1, 25-2, 17-1, 17-2, 26-1, 26-2, 14-1, 14-2, 27-1, 27-2, 28-1, 28-2, 29-1, 29-2, 30-1, 30-2, 31-1, 31-2, 32-1, 32-2, 33-3, 33-4, 34-1, 35-1, 35-2, 33-1, 33-2, 34-2, 36-1, 36-2, 37-1, 37-2, 34-3, 34-4, 38-1, 38-2, 39-1, 39-2, 40-1, 40-2, 41-1, 41-2, 42-1, 42-2, 43-1, 43-2, 44-1, 44-2, 45-1, 45-2, 46-1, 46-2, 47-1, 47-2, 48-1, 48-2, 49-1, 49-2, 50-1, 50-2, 51-2, 51-3, 51-6, 52-2, 52-3, 52-6, 53-1, 53-2, 54-1, 54-2, 51-5, 52-5, 55-1, 55-2, 56-1, 56-2, 57-1, and 57-2, or a single stereoisomer or mixture of stereoisomers thereof.

In certain embodiments, the compound or salt thereof is selected from the group consisting of the compounds of Table 2 consisting of Examples 2, 2-4, 4, 5, 11, 13, 15, 16, 19, 20, 21, 22-1, 22-4, 23, 24, 26, 28, 29, 30, 31, 32, 33, 34, 35, 36, 37, 38, 39, 40, 41, 42, 43, 44, 45, 46, 47, 48, 49, 50, 51-1, 51-4, 52-1, 52-4, 53, 54, 55, 56, and 57, or a single stereoisomer or mixture of stereoisomers thereof, as defined herein.

In certain embodiments, the compound or salt thereof is selected from the group consisting of the compounds of Table 2 consisting of Examples 12, 12-1, 12-2, 13, 13-1, 13-2, 14, 14-1, 14-2, 15-1, 16, 16-1, 16-2, 18, 23, 23-1, 23-2, 31, 31-1, and 31-2, or a single stereoisomer or mixture of stereoisomers thereof, as defined herein.

### Pharmaceutical Compositions

In certain embodiments, optionally in combination with any or all of the above various embodiments, provided herein is a pharmaceutical composition comprising of a compound disclosed hereinor a pharmaceutically acceptable salt thereof, and one or more pharmaceutically acceptable excipients.

In certain embodiments, the pharmaceutical composition comprises a compound of Formula I or II, or a compound of Table 1, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-338, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-141 and 143-338, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-267, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-141 and 143-267, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 268-338, or stereoisomers thereof, or a pharmaceutically acceptable salt thereof, and one or more pharmaceutically acceptable excipients.

In certain embodiments, the pharmaceutical composition comprises a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Iab, Iac, Iad, Iae, or Iaf, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-2, 4-17, 19-21, 23-26, 28-36, 38-39, 42-43, 45, 47, 49, 51-64, 66-69, 71-242, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 1 consisting of Examples 1-2, 4-17, 19-21, 23-26, 28-36, 38-39, 42-43, 45, 47, 49, 51-64, 66-69, 71-141, and 143-242, or stereoisomers thereof, or a pharmaceutically acceptable salt thereof, and one or more pharmaceutically acceptable excipients.

In certain embodiments, the pharmaceutical composition comprises a compound of Formula II, IIa, IIb, IIc, IId, or IIe, or a pharmaceutically acceptable salt thereof, and one or more pharmaceutically acceptable excipients. In certain embodiments, the pharmaceutical composition comprises a compound selected from the group consisting of the compounds of Table 1 consisting of Examples 3, 18, 22, 27, 37, 40, 41, 44, 46, 48, 50, 65, 70, and 243-267, or a pharmaceutically acceptable salt thereof, and one or more pharmaceutically acceptable excipients.

In certain embodiments, the pharmaceutical composition comprises a compound of Formula VII, or a compound of Table 2, or stereoisomers thereof, or a pharmaceutically acceptable salt thereof, and one or more pharmaceutically acceptable excipients. In certain embodiments, the pharmaceutical composition comprises a compound selected from the group consisting of the compounds of Table 2 consisting of Examples 1-57, or stereoisomers thereof, or a pharmaceutically acceptable salt thereof, and one or more pharmaceutically acceptable excipients.

In certain embodiments, the pharmaceutical composition comprises a compound of Formula VII, VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, VIIaf, VIIag, VIIah, VIIai, or VIIaj, or stereoisomers thereof, or a pharmaceutically acceptable salt thereof, and one or more pharmaceutically acceptable excipients.

In certain embodiments, the pharmaceutical composition comprises a compound selected from the group consisting of the compounds of Table 2 consisting of Examples 2-2, 6, 6-2, 7, 7-2, 8, 8-2, 9, 9-2, 10, 10-2, 11-2, 14, 15-2, 17, 17-1, 19-2, 19-3, 20-1, 21-2, 22-2, 22-3, 24-1, 25-1, 27-2, 28-2, 29-2, 30-2, 31-2, 32-2, 33-1, 33-4, 34-1, 34-3, 35-2, 36-2, 37-2, 38-2, 39-1, 40-2, 41-2, 42-1, 43-2, 44-2, 45-2, 46-1, 47-2, 48-2, 49-2, 50-1, 51-2, 52-2, 53-2, 54-2, 52-5, 56-2, and 57-2, or stereoisomers thereof, or a pharmaceutically acceptable salt thereof, and one or more pharmaceutically acceptable excipients.

Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. The suitability of a particular excipient may also depend on the specific active ingredients in the dosage form.

### Formulation and Administration

All the compounds and pharmaceutical compositions provided herein can be used in all the methods provided herein. For example, the compounds and pharmaceutical compositions provided herein can be used in all the methods for treatment of all diseases, disorders or conditions provided herein. Thus, the compounds and pharmaceutical compositions provided herein are for use as a medicament. The compounds and pharmaceutical compositions provided herein are for use in a method for the treatment of a disease or disorder that is mediated by the enzyme CGT. The compounds and pharmaceutical compositions provided herein are for use in a method for the treatment of a disease or disorder in which inhibition of the enzyme CGT ameliorates or treats the disease or disorder. A compound provided herein is a compound of Formula I, Ia, Ib, Ic, Id, Ie, If, Ig, Ih, Ii, Ij, Ik, II, Im, In, Io, Ip, Iq, Ir, Is, It, Iu, Iv, Iw, Ix, Iy, Iz, Iaa, Iab, Iac, Iad, Iae, or Iaf, or a compound of Formula II, IIa, IIb, IIc, IId, or IIe, or a compound of Table 1, or stereoisomers thereof, and additionally optionally a pharmaceutically acceptable salt thereof. For example, a compound provided herein is also a compound of Formula VII, VIIa, VIIb, VIIc, VIId, VIIe, VIIf, VIIg, VIIh, VIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, VIIaf, VIIag, VIIah, VIIai, or VIIaj, or a compound of Table 2, or stereoisomers thereof, and additionally optionally a pharmaceutically acceptable salt thereof. In certain embodiments, the compounds described herein are used in the preparation or manufacture of medicaments for the treatment of a disease or disorder that is mediated by the enzyme CGT or in which inhibition of the enzyme CGT ameliorates or treats the disease or disorder. In certain embodiments, provided is a method for treating any of the diseases or disorders described herein comprising administering to a subject having the disease or disorder a compound according to any of the various embodiments described herein or a pharmaceutical composition according to any of the various embodiments described herein. In certain embodiments, provided is a method for treating any of the diseases or disorders described herein comprising administering to a subject in need of treatment thereof a compound according to any of the various embodiments described herein or a pharmaceutical composition according to any of the various embodiments described herein.

In certain embodiments, provided herein is a method of treating a disease or disorder ameliorated by the inhibition of CGT comprising administering to a subject having the disease or disorder a therapeutically effective amount of a compound provided herein.

In certain embodiments, the method comprises administering to a subject having the disease or disorder a compound of Formula I or II, or a compound of Table 1, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-338, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-141 and 143-338, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-267, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-141 and 143-267, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 268-338, or stereoisomers thereof, and additionally optionally a pharmaceutically acceptable salt thereof.

In certain embodiments, the method comprises administering to a subject having the disease or disorder a therapeutically effective amount of a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Iab, Iac, Iad, Iae, or Iaf, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-2, 4-17, 19-21, 23-26, 28-36, 38-39, 42-43, 45, 47, 49, 51-64, 66-69, 71-242, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-2, 4-17, 19-21, 23-26, 28-36, 38-39, 42-43, 45, 47, 49, 51-64, 66-69, 71-141, and 143-242, or stereoisomers thereof, and additionally optionally a pharmaceutically acceptable salt thereof.

In certain embodiments, the method comprises administering to a subject having the disease or disorder a therapeutically effective amount of a compound of Formula II, IIa, IIb, IIc, IId, or IIe, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 3, 18, 22, 27, 37, 40, 41, 44, 46, 48, 50, 65, 70, and 243-267, and additionally optionally a pharmaceutically acceptable salt thereof.

In certain embodiments, provided herein is a method of treating a disease or disorder ameliorated by the inhibition of CGT comprising administering to a subject having the disease or disorder a pharmaceutical composition comprising a therapeutically effective amount of a compound provided herein.

In certain embodiments, the method comprises administering to a subject having the disease or disorder a pharmaceutical composition comprising a compound of Formula I or II, or a compound of Table 1, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-338, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-141 and 143-338, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-267, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-141 and 143-267, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 of Examples 238-338, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound of Formula I, Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Iab, Iac, Iad, Iae, or Iaf, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-2, 4-17, 19-21, 23-26, 28-36, 38-39, 42-43, 45, 47, 49, 51-64, 66-69, 71-242, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 1-2, 4-17, 19-21, 23-26, 28-36, 38-39, 42-43, 45, 47, 49, 51-64, 66-69, 71-141, and 143-242, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound of Formula II, IIa, IIb, IIc, IId, or IIe, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 1 consisting of Examples 3, 18, 22, 27, 37, 40, 41, 44, 46, 48, 50, 65, 70, and 243-267, and additionally optionally a pharmaceutically acceptable salt thereof, and a pharmaceutically acceptable excipient.

In certain embodiments, the method comprises administering to a subject having the disease or disorder a compound of Formula VII, or a compound of Table 2, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 1 consisting of Examples 1-57, or stereoisomers thereof, and additionally optionally a pharmaceutically acceptable salt thereof.

In certain embodiments, the method comprises administering to a subject having the disease or disorder a therapeutically effective amount of a compound of Formula VII, VIIa, VIIb, VIIc, VIId, VIIe, VIIf, VIIg, VIIh, VIIi, VIIj, VIIk, VII, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, VIIaf, VIIag, VIIah, VIIai, or VIIaj, or stereoisomers thereof, and additionally optionally a pharmaceutically acceptable salt thereof.

In certain embodiments, the method comprises administering to a subject having the disease or disorder a therapeutically effective amount of a compound selected from the group consisting of the compounds of Table 2 consisting of Examples 2-2, 6, 6-2, 7, 7-2, 8, 8-2, 9, 9-2, 10, 10-2, 11-2, 14, 15-2, 17, 17-1, 19-2, 19-3, 20-1, 21-2, 22-2, 22-3, 24-1, 25-1, 27-2, 28-2, 29-2, 30-2, 31-2, 32-2, 33-1, 33-4, 34-1, 34-3, 35-2, 36-2, 37-2, 38-2, 39-1, 40-2, 41-2, 42-1, 43-2, 44-2, 45-2, 46-1, 47-2, 48-2, 49-2, 50-1, 51-2, 52-2, 53-2, 54-2, 52-5, 56-2, and 57-2, or stereoisomers thereof, and additionally optionally a pharmaceutically acceptable salt thereof.

In certain embodiments, the method comprises administering to a subject having the disease or disorder a pharmaceutical composition comprising a therapeutically effective amount of a compound of Formula VII, or a compound of Table 2, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 2 consisting of Examples 1-57, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound of Formula VII, VIIa, VIIb, VIIc, VIId, VIIe, VIIf, VIIg, VIIh, VIIi, VIIj, VIIk, VIIl, VIIm, VIIn, VIIo, VIIp, VIIq, VIIr, VIIs, VIIt, VIIu, VIIv, VIIw, VIIx, VIIy, VIIz, VIIaa, VIIab, VIIac, VIIad, VIIae, VIIaf, VIIag, VIIah, VIIai, or VIIaj, or stereoisomers thereof, and additionally optionally a pharmaceutically acceptable salt thereof, and a pharmaceutically acceptable excipient.

In certain embodiments, the method comprises administering to a subject having the disease or disorder a pharmaceutical composition comprising a therapeutically effective amount of a compound selected from the group consisting of the compounds of Table 2 consisting of Examples 2-2, 6, 6-2, 7, 7-2, 8, 8-2, 9, 9-2, 10, 10-2, 11-2, 14, 15-2, 17, 17-1, 19-2, 19-3, 20-1, 21-2, 22-2, 22-3, 24-1, 25-1, 27-2, 28-2, 29-2, 30-2, 31-2, 32-2, 33-1, 33-4, 34-1, 34-3, 35-2, 36-2, 37-2, 38-2, 39-1, 40-2, 41-2, 42-1, 43-2, 44-2, 45-2, 46-1, 47-2, 48-2, 49-2, 50-1, 51-2, 52-2, 53-2, 54-2, 52-5, 56-2, and 57-2, and additionally optionally a pharmaceutically acceptable salt thereof, and a pharmaceutically acceptable excipient.

In certain embodiments, the disease or disorder is medicated by the enzyme ceramide galactosyltransferase (CGT).

In certain embodiments, the disease or disorder is a lysosomal storage disease. Those of skill in the art will understand that “lysosomal storage disease” and “lysosomal deficiency disease” refer to the same disease or disorder. Examples of lysosomal storage diseases include, for example, Krabbe disease and Metachromatic Leukodystrophy (MLD). In certain embodiments, the disease or disorder is Krabbe disease. In certain embodiments, the disease or disorder is MLD.

The compounds or compositions disclosed herein can be administered in therapeutically effective amounts via any of the usual and acceptable modes known in the art, either singly or in combination with another therapeutic agent. The compounds are typically administered as pharmaceutical compositions by any route which makes the compound bioavailable. In certain embodiments, the composition is a solid formulation adapted for oral administration. In certain embodiments, the composition is a tablet, powder, or capsule; or the composition is a tablet. In certain embodiments, the composition is a liquid formulation adapted for oral administration. In certain embodiments, the composition is a liquid formulation adapted for parenteral administration. In certain embodiments, the composition is a solution, suspension, or emulsion; or the composition is a solution. In certain embodiments, solid form compositions can be converted, shortly before use, to liquid form compositions for either oral or parenteral administration. These particular solid form compositions are provided in unit dose form and as such are used to provide a single liquid dosage unit. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

The dosages may be varied depending on the requirement of the patient, the severity of the disease or disorder being treating and the particular compound and/or composition being employed. Determination of the proper dosage can be determined by one skilled in the medical arts. The total daily dosage may be divided and administered in portions throughout the day or by means providing continuous delivery. In certain embodiments, the compounds are administered to a subject at a daily dosage of between 0.01 to about 50 mg/kg of body weight. In other embodiments, the dose is from 1 to 1000 mg/day. In certain embodiments, the daily dose is from 1 to 750 mg/day; or from 10 to 500 mg/day.

In certain embodiments, the pharmaceutical composition is in unit dosage form. The composition can be subdivided into unit doses containing appropriate quantities of the active component(s). The unit dosage form can be a tablet, capsule, or powder in a vial or ampule, or it may be the appropriate number of any of these in a packaged form. The unit dosage form can be a packaged form, the package containing discrete quantities of composition such as packeted tablets, capsules, or powders in vials or ampules. The quantity of active compound(s) in a unit dose of the composition may be varied or adjusted from about 1 mg to about 100 mg, or from about 1 mg to about 50 mg, or from about 1 mg to about 25 mg.

The compounds or pharmaceutical compositions disclosed herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Preparation of Compounds

The following are illustrative examples of how the compounds disclosed herein can be prepared and tested. Although the examples can represent only some embodiments, it should be understood that the following examples are illustrative and not limiting.

In a further aspect, it is provided a method of making a compound, comprising synthesizing a compound as any of the various embodiments described above or below. Examples of the method are further described in the Examples.

Compounds disclosed herein are commercially available or can be readily prepared from commercially available starting materials according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Synthesis of some of the compounds are exemplified in detail below.

In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column.

Materials were obtained from commercial suppliers and were used without further purification. Air or moisture sensitive reactions were conducted under argon atmosphere using oven-dried glassware and standard syringe/septa techniques. ¹H NMR spectra were measured at 400 MHz unless stated otherwise and data were reported as follows in ppm (δ) from the internal standard (TMS, 0.0 ppm): chemical shift (multiplicity, integration, coupling constant in Hz).

### Preparation of Compounds of Formulas I and II

The following are illustrative examples of how the compounds of Formula I (e.g., Formulas Ia, Ib, Ic, Id, Ie, If, Ig, Ih, Ii, Ij, Ik, Il, Im, In, Io, Ip, Iq, Ir, Is, It, Iu, Iv, Iw, Ix, Iy, Iz, Iaa, Iab, lac, lad, Iae, and Iaf) and compounds of Formula II (e.g., IIa, IIb, IIc, IId, and Ile), and/or stereoisomers thereof, can be prepared.

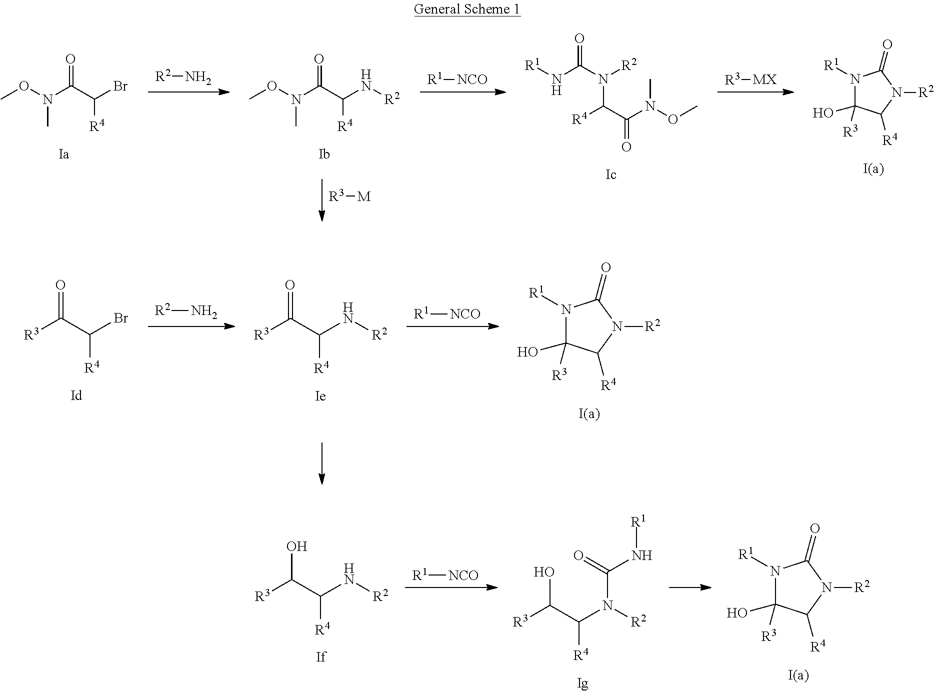

A compound of Formula I(a) (where R⁵ is hydrogen and all other groups are as defined in the Summary of the Invention for a compound of Formula I or according to any of the embodiments disclosed herein) can be prepared according to General Scheme 1.

An intermediate of formula Ib can be prepared using standard nucleophilic amino substitution conditions. More specifically, an intermediate of formula Ia, which can be prepared using procedures disclosed herein or are known to one of ordinary skill in the art, can be reacted with an amine of formula R²—NH₂ in a solvent such as DMF, EtOH, DMSO, or THF, optionally in the presence of NaI and a base such as NaOAc, and at ambient temperature or up to 100° C.

An intermediate of formula Ic can be prepared using standard urea formation conditions, which can include procedures disclosed herein or those known to one of ordinary skill in the art. More specifically, the intermediate of formula Ib can be reacted with an isocyanate of formula R¹—NCO in a solvent such as DCM, toluene, or pyridine, optionally in the presence of a base such as DIPEA or TEA, to yield the intermediate of formula Ic.

The intermediate of formula Ic can then be reacted with a Grignard reagent of formula R³-MX or an organolithium reagent of formula R³—Li, which are commercially available or can be made by using procedures disclosed herein or are known to one of ordinary skill in the art, in a solvent such as Et₂O, or THF to yield the compound of Formula I(a).

The intermediate of formula Ib can also be treated with a Grignard reagent of formula R³-MX or an organolithium reagent of formula R³—Li in a solvent such as Et₂O, or THF, optionally in the presence of a base such as DIPEA, or TEA to yield an intermediate of formula Ie.

The intermediate of formula Ie can also be prepared using standard nucleophilic amines substitution conditions. More specifically, an intermediate of formula Id, which can be prepared using procedures disclosed herein or are known to one of ordinary skill in the art, can be reacted with an amine of formula R²—NH₂ in a solvent such as EtOH, NMP, DMSO, or DMF, optionally in the presence of a base such as carbonate, bicarbonate, DIPEA, or TEA.

The intermediate of formula Ie can then be reacted with an isocyanate of formula R¹—NCO in a solvent such as DCM, toluene, or pyridine, optionally in the presence of a base such as DIPEA or TEA, to yield the Compound of Formula I(a).

The compound of Formula I(a) can also be made by an alternative route using intermediate of formula Ie through intermediates formula If and Ig.

The intermediate of formula Ie can be reduced to the corresponding alcohol by using standard reduction conditions for converting a ketone to an alcohol, which are disclosed herein or are known to one of ordinary skill in the art. More specifically, the intermediate of formula Ie can be treated with a reducing agent such as sodium borohydride, in a solvent such as EtOH, or THF to yield an intermediate of formula If.

The intermediate of formula If can then be reacted with an isocyanate of formula R¹—NCO in a solvent such as DCM, toluene, or pyridine, optionally in the presence of a base such as DIPEA or TEA, to yield an intermediate of formula Ig.

The compound of Formula I(a) can be prepared using standard oxidation conditions for converting an alcohol to a ketone, which are disclosed herein or are known to one of ordinary skill in the art. More specifically, the intermediate of formula Ig can be treated with an oxidizing reagent such as Dess-Martin periodinane (DMP) in a solvent such as DCM or CHCl₃, in the presence of a base such as sodium bicarbonate.

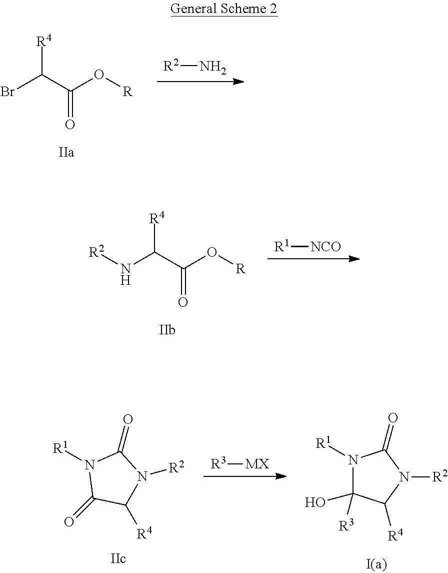

The compound of Formula I(a) (where R⁵ is hydrogen and all other groups are as defined in the Summary of the Invention for a compound of Formula I or according to any of the embodiments disclosed herein) can also be prepared according to General Scheme 2.

An intermediate of formula IIb can be prepared using standard nucleophilic amino substitution conditions. More specifically, an intermediate of formula IIa, which can be prepared using procedures disclosed herein or are known to one of ordinary skill in the art, can be reacted with an amine of formula R²—NH₂ in a solvent such as acetone, EtOH, or THF, optionally in the presence of NaI and a base such as NaHCO₃, K₂CO₃, or NaOAc.

An intermediate of formula IIc can be prepared using standard urea formation conditions, which can include procedures disclosed herein or those known to one of ordinary skill in the art. More specifically, the intermediate of formula IIb can be reacted with an isocyanate of formula R¹—NCO in a solvent such as DCM, toluene, or pyridine, optionally in the presence of a base such as DIPEA or TEA, to yield the intermediate of formula IIc.

The intermediate of formula IIc can then be reacted with a Grignard reagent of formula R³-MX or an organolithium reagent of formula R³—Li in a solvent such as Et₂O, or THF to yield the compound of Formula I(a).

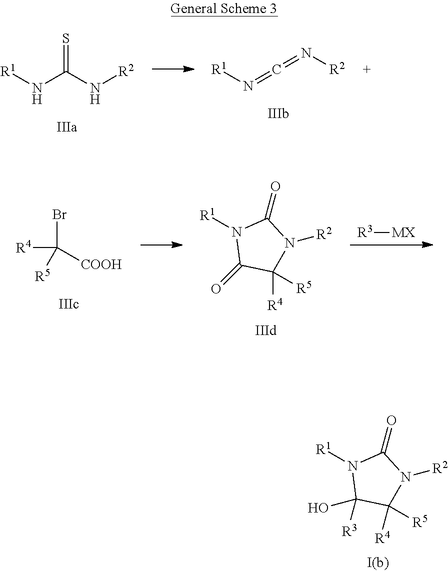

A compound of Formula I(b) (where R⁵ is alkyl, alkoxy, or cycloalkyl and all other groups are as defined in the Summary of the Invention for a compound of Formula I or according to any of the embodiments disclosed herein) can also be prepared according to General Scheme 3.

An intermediate of formula IIIb can be prepared using standard carbodiimide formation conditions. More specifically, an intermediate of formula IIIa, which can be prepared using procedures disclosed herein or are known to one of ordinary skill in the art, can be treated with iodine in a solvent such as EtOAc, or DCM, optionally in the presence of a base such as Et₃N, or DIPEA.

The intermediate of formula IIIb can be reacted with IIIc, which can be prepared using procedures disclosed herein or are known to one of ordinary skill in the art, in a solvent such as 1,4-dioxane, or DCM, optionally in the presence of a base such as s-collidine, or Et₃N, or DIPEA, to yield an intermediate of formula IIId.

The intermediate of formula IIId can then be reacted with an organolithium reagent of formula R³—Li in a solvent such as Et₂O, or THF to yield the compound of Formula I(b).

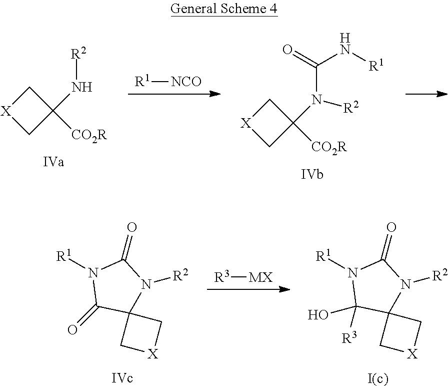

A compound of Formula I(c) (where R⁴ and R⁵ and the carbon to which they are attached combine to form spirocycloalkyl or spiroheterocycloalkyl and all other groups are as defined in the Summary of the Invention for a compound of Formula I or according to any of the embodiments disclosed herein) can be prepared according to General Scheme 4.

An intermediate of formula IVb can be prepared using standard urea formation conditions, which can include procedures disclosed herein or those known to one of ordinary skill in the art. More specifically, an intermediate of formula IVa, which can be prepared using procedures disclosed herein or are known to one of ordinary skill in the art, can be reacted with an isocyanate of formula R¹—NCO in a solvent such as DCM, toluene, or pyridine, optionally in the presence of a base such as DIPEA or TEA, to yield the intermediate of formula IVb.

The intermediate of formula IVb can then be treated with a base such as ammonia in a solvent such as MeOH, or EtOH to yield an intermediate of formula IVc.

The intermediate of formula IVc can then be reacted with a Grignard reagent of formula R³-MX or an organolithium reagent of formula R³—Li in a solvent such as Et₂O, or THF to yield the compound of Formula I(c).

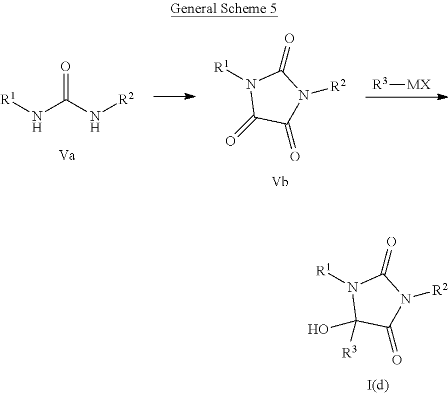

A compound of Formula I(d) (where R⁴ and R⁵ and the carbon to which they are attached combine to form carbonyl, and all other groups are as defined in the Summary of the Invention for a compound of Formula I or according to any of the embodiments disclosed herein) can be prepared according to General Scheme 5.

An intermediate of formula Vb can be prepared using procedures disclosed herein or are known to one of ordinary skill in the art. More specifically, an intermediate of formula Va, which can be prepared using procedures disclosed herein or are known to one of ordinary skill in the art, can be reacted with oxalyl chloride in a solvent such as DCM, toluene, or THF, optionally in the presence of a base such as DIPEA, or TEA, to yield the intermediate of formula Vb.

The intermediate of formula Vb can then be reacted with a Grignard reagent of formula R³-MX or an organolithium reagent of formula R³—Li in a solvent such as Et₂O, or THF to yield the compound of Formula I(d).

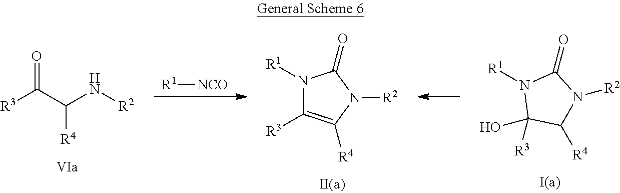

A compound of Formula II(a) (where all groups are as defined in the Summary of the Invention for a compound of Formula I or according to any of the embodiments disclosed herein) can be prepared according to General Scheme 6.

The compound of Formula II(a) can be prepared using standard urea formation conditions, which can include procedures disclosed herein or those known to one of ordinary skill in the art. More specifically, an intermediate of formula VIa, which can be prepared using procedures disclosed herein or are known to one of ordinary skill in the art, can be reacted with an isocyanate of formula R¹—NCO in a solvent such as DCM, toluene, or pyridine, optionally in the presence of a base such as DIPEA or TEA, and then treated with an acid such as TFA, HCl, or a Lewis acid, in a solvent such as TFA or DCM.

The compound of Formula II(a) can also be prepared using elimination of water conditions, which are disclosed herein or are known to one of ordinary skill in the art. More specifically, the intermediate of formula I(a) can be treated with an acid such as TFA, HCl, or a Lewis acid, in a solvent such as TFA or DCM.

### SYNTHETIC EXAMPLES (COMPOUNDS OF FORMULAS I AND II)

### Example 1

### Synthesis of 1,3-bis(4-bromophenyl)-4-hydroxy-4-phenylimidazolidin-2-one

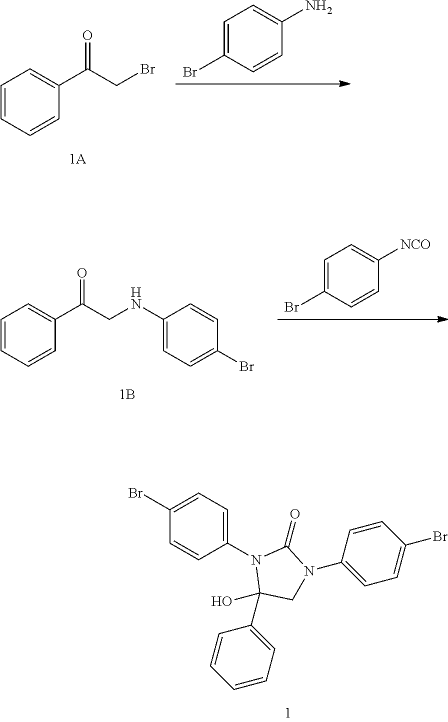

To a mixture of 4-bromoaniline (1.68 g, 20.0 mmol) and sodium bicarbonate (1.68 g, 20.0 mmol) in ethanol (100 mL) was added Compound 1A (3.98 g, 20.0 mmol) at 25° C. under nitrogen. The mixture was stirred vigorously at the same temperature for 6 hours, diluted with water (10 mL), and extracted with ethyl acetate (100 mL×3). The combined organic layers was washed with water (50 mL×2), dried over anhydrous sodium sulfate, filtered, and concentrated under reduced pressure to give Compound 1B: LC-MS (ESI) m/z: 290 [M+H]⁺.

To a solution of Compound 1B (290 mg, 1.0 mmol) in dichloromethane (15 mL) was added 1-bromo-4-isocyanatobenzene (198 mg, 1.0 mmol). The mixture was stirred at room temperature overnight. The resulting mixture was concentrated under reduced pressure to leave a crude product, which was purified by preparative HPLC to furnish Compound 1: LC-MS (ESI) m/z: 487 [M+H]+; ¹H-NMR (Acetone-d₆, 400 MHz): δ (ppm) 4.20 (dd, J=32.0 Hz, J₂=10.8 Hz, 2H), 6.72 (s, 1H), 7.28-7.36 (m, 5H), 7.44-7.53 (m, 4H), 7.67-7.71 (m, 4H).

### Example 2

### Synthesis of 1,3-bis(4-bromophenyl)-4-hydroxy-4-methylimidazolidin-2-one

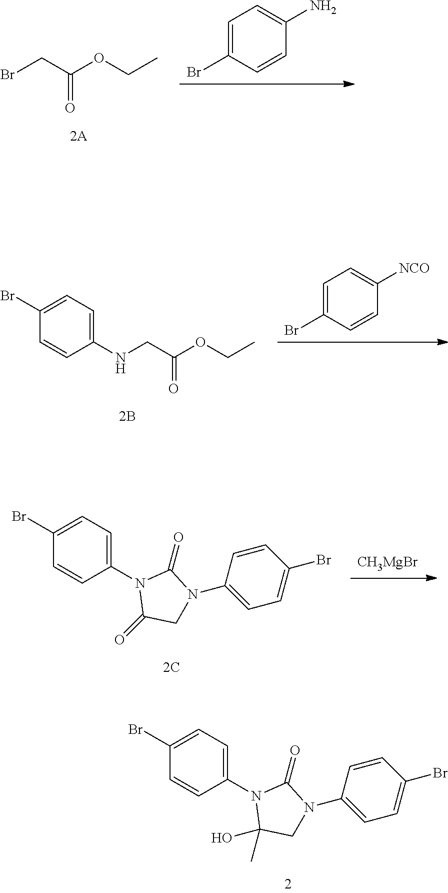

To a solution of Compound 2A (8.35 g, 50.0 mmol) in anhydrous ethanol (100 mL) was added 4-bromoaniline (8.60 g, 50.0 mmol), anhydrous NaOAc (4.1 g, 50.0 mmol), and NaI (7.5 g, 50.0 mmol). The mixture was heated at reflux under nitrogen for 4 hours. The reaction mixture was filtered while hot and the filtrate was cooled in an ice bath for 30 minutes. The resulting precipitate was collected and re-crystallized from ethanol-hexane to afford a pure Compound 2B: LC-MS (ESI) m/z: 258 [M+H]⁺; ¹H-NMR (DMSO-d₆, 400 MHz): δ (ppm) 1.18 (t, J=7.2 Hz, 3H), 3.87 (s, 2H), 4.10 (q, J=7.2 Hz, 2H), 6.22 (br, 1H), 6.51 (d, J=8.8 Hz, 2H), 7.20 (d, J=8.8 Hz, 2H).

To a solution of Compound 2B (2.58 g, 10.0 mmol) in toluene was added 1-bromo-4-isocyanatobenzene (1.98 g, 10.0 mmol). The mixture was heated at reflux overnight. The reaction mixture was concentrated under reduced pressure to give Compound 2C: LC-MS (ESI) m/z: 409 [M+H]⁺; ¹H-NMR (CDCl₃, 400 MHz): δ (ppm) 4.46 (s, 2H), 7.35-7.37 (m, 2H), 7.49-7.53 (m, 4H), 7.62-7.64 (m, 2H).

To a solution of Compound 2C (210 mg, 0.5 mmol) in THF (10 mL) was dropped a solution of methylmagnesium bromide in diethyl ether (3.0 M, 0.5 mL, 1.5 mmol) at 10° C. under nitrogen. The mixture was stirred at 10° C. overnight. The reaction mixture was quenched with saturated NH₄Cl solution (3 mL) and extracted with ethyl acetate (10 mL×3). The combined organic layers was washed with water (5 mL) and brine (5 mL), dried over anhydrous sodium sulfate, filtered, and concentrated to leave a crude product, which was purified by preparative HPLC to afford Compound 2: LC-MS (ESI) m/z: 425 [M+H]⁺; ¹H-NMR (Acetone-d₆, 400 MHz): δ (ppm) 1.59 (s, 3H), 4.02 (dd, J₁=48.4 Hz, J₂=10.0 Hz, 2H), 5.82 (s, 1H), 7.48-7.51 (m, 4H), 7.57-7.66 (m, 4H).

### Example 3

### Synthesis of 3-(1,3-bis(4-chlorophenyl)-5-methyl-2-oxo-2,3-dihydro-1H-imidazol-4-yl)benzonitrile

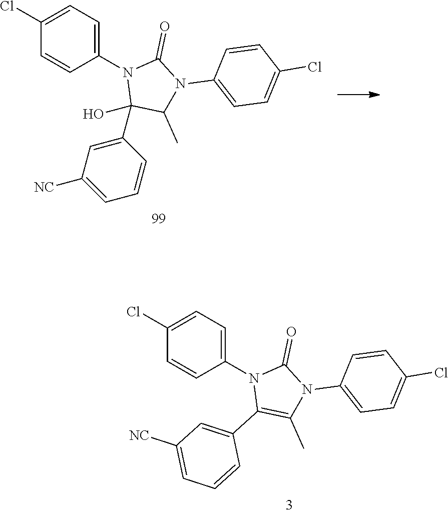 [SMILES: CC1N(c2ccc(Cl)cc2)C(=O)N(c2ccc(Cl)cc2)C1(O)c1cccc(C#N)c1 ; Cc1c(-c2cccc(C#N)c2)n(-c2ccc(Cl)cc2)c(=O)n1-c1ccc(Cl)cc1]

Compound 3 was synthesized by employing the procedure described for Compound 255 using Compound 99 lieu of Compound 1. LC-MS (ESI) m/z: 420 [M+H]⁺; ¹H-NMR (DMSO-d₆, 400 MHz): δ (ppm) 2.03 (s, 3H), 7.19-7.21 (m, 2H), 7.37-7.40 (m, 1H), 7.44-7.46 (m, 2H), 7.51-7.55 (m, 3H), 7.62-7.66 (m, 3H), 7.74-7.76 (m, 1H).

### Example 4

### Synthesis of 1,3-bis(4-bromophenyl)-4-ethyl-4-hydroxyimidazolidin-2-one

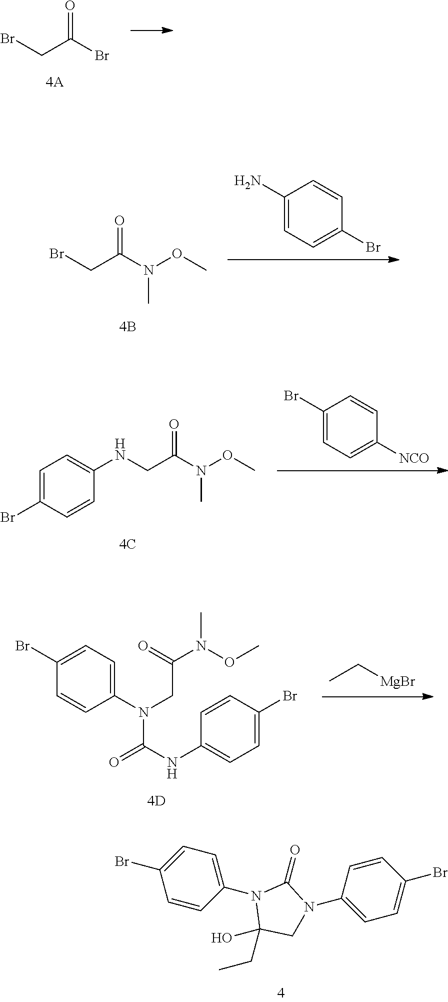

A mixture of Compound 4A (13.6 mL, 160 mmol), potassium carbonate (44 g, 320 mmol), and N,O-dimethylhydroxylamine hydrochloride (15.2 g, 160 mmol) in CH₃CN (100 mL) was stirred at 20° C. for 3 hours. The mixture was filtered and the filtrate was concentrated under reduced pressure. The residue was dissolved in dichloromethane (100 mL), washed with water (200 mL) and brine (200 mL), dried over anhydrous sodium sulfate, filtered, and concentrated to afford a crude Compound 4B: LC-MS (ESI) m/z: 182 [M+1]⁺.

Compounds 4C, 4D, and 4 were synthesized by employing the procedures described for Compounds 2B, 2C, and 2 using Compounds 4B, 4C, Compound 4D, ethylmagnesium chloride, and stirred at 0° C. in lieu of Compounds 2A, 2B, Compound 2C, methylmagnesium bromide, and stirred at 10° C. Compound 4C: LC-MS (ESI) m/z: 273 [M+1]⁺; ¹H-NMR (CDCl₃, 400 MHz): δ (ppm) 3.25 (s, 3H), 3.76 (s, 3H), 3.97 (s, 2H), 6.52 (d, J=8.8 Hz, 2H), 7.26 (d, J=8.8 Hz, 2H). Compound 4D: LC-MS (ESI) m/z: 470 [M+1]⁺; ¹H-NMR (CDCl₃, 400 MHz): δ (ppm) 3.20 (s, 3H), 3.75 (s, 3H), 4.55 (s, 2H), 6.29 (s, 1H), 7.19 (d, J=8.8 Hz, 2H), 7.33 (d, J=8.8 Hz, 2H), 7.43 (d, J=8.8 Hz, 2H), 7.59 (d, J=8.8 Hz, 2H). Compound 4: LC-MS (ESI) m/z: 439 [M+H]⁺, 899 [2M+Na]⁺; ¹H-NMR (CDCl₃, 400 MHz): δ (ppm) 0.88 (t, J=7.2 Hz, 3H), 1.79-1.85 (m, 2H), 3.47-3.53 (m, 1H), 3.73 (d, J=6.4 Hz, 1H), 3.89 (d, J=6.4 Hz, 1H), 7.30 (d, J=8.8 Hz, 2H), 7.41 (s, 4H), 7.45 (d, J=8.4 Hz, 2H).

### Example 5

### Synthesis of 1,3-bis(4-bromophenyl)-4-(5-chlorothiophen-2-yl)-4-hydroxyimidazolidin-2-one

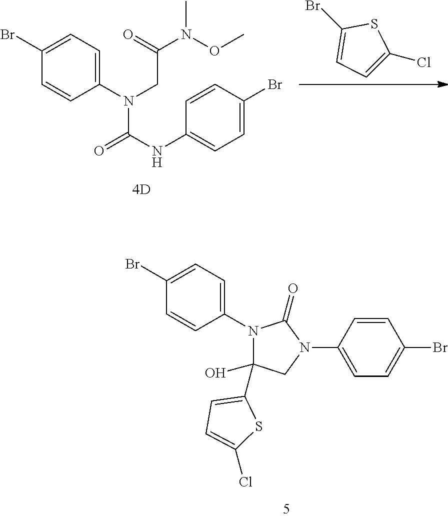 [SMILES: CON(C)C(=O)CN(C(=O)Nc1ccc(Br)cc1)c1ccc(Br)cc1 ; Clc1ccc(Br)s1 ; O=C1N(c2ccc(Br)cc2)CC(O)(c2ccc(Cl)s2)N1c1ccc(Br)cc1]

To a solution of 2-bromo-5-chlorothiophene (198 mg, 1 mmol) in dry THF (10 mL) at −78° C. was dropped a solution of n-BuLi in THF (2.5 M, 0.4 mL, 1 mmol) over 5 minutes. After stirred at −78° C. for 0.5 hour, to the mixture at −20° C. was dropped a solution of Compound 4D (118 mg, 0.25 mmol) in anhydrous THF (5 mL). The mixture was stirred at 0° C. for 2 hours, quenched with water (20 mL), and extracted with ethyl acetate (20 mL×3). The combined organic layers was washed with brine (40 mL), dried over anhydrous sodium sulfate, filtered, and concentrated to afford a crude product, which was purified with preparative HPLC to furnish Compound 5: LC-MS (ESI) m/z: 527 [M+1]⁺; ¹H-NMR (CDCl₃, 400 MHz): δ (ppm) 4.06 (s, 2H), 4.68 (s, 1H), 6.70 (d, J=4 Hz, 1H), 6.74 (d, J=4 Hz, 1H), 7.18 (d, J=8.8 Hz, 2H), 7.29-7.33 (m, 4H), 7.39 (d, J=8.8 Hz, 2H).

### Example 6

### Synthesis of 1,3-bis(4-bromophenyl)-4-butyl-4-hydroxyimidazolidin-2-one

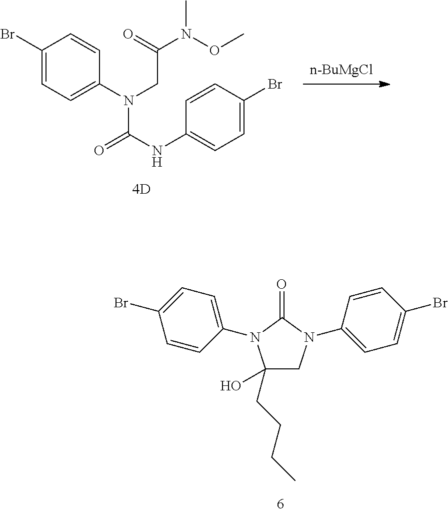 [SMILES: "n-BuMgCl" ; CON(C)C(=O)CN(C(=O)Nc1ccc(Br)cc1)c1ccc(Br)cc1 ; CCCCC1(O)CN(c2ccc(Br)cc2)C(=O)N1c1ccc(Br)cc1]

Compound 6 was synthesized by employing the procedure described for Compound 2 using Compound 4D, n-butylmagnesium chloride, and at 0° C. in lieu of Compound 2C, methylmagnesium chloride, and at 10° C. LC-MS (ESI) m/z: 467 [M+H]⁺; ¹H-NMR (CD₃OD, 400 MHz): δ (ppm) 0.83 (t, J=7.2 Hz, 3H), 1.18-1.54 (m, 4H), 1.76-1.94 (m, 2H), 3.88 (d, J=10.0 Hz, 1H), 4.17 (d, J=10.0 Hz, 1H), 5.87 (s, 1H), 7.47-7.61 (m, 6H), 7.65-7.71 (m, 2H).

### Example 7

### Synthesis of 1,3-bis(4-bromophenyl)-4-hydroxy-4-(thiophen-2-yl)imidazolidin-2-one

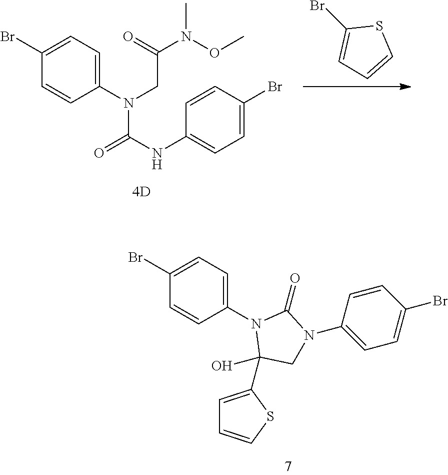 [SMILES: CON(C)C(=O)CN(C(=O)Nc1ccc(Br)cc1)c1ccc(Br)cc1 ; Brc1cccs1 ; O=C1N(c2ccc(Br)cc2)CC(O)(c2cccs2)N1c1ccc(Br)cc1]

Compound 7 was synthesized by employing the procedure described for Compound 5 using 2-bromothiophene in lieu of 2-bromo-5-chlorothiophene. LC-MS (ESI) m/z: 493; ¹H-NMR (DMSO-d₆, 400 MHz): δ (ppm) 4.13-4.30 (m, 2H), 6.92-6.95 (m, 1H), 7.15-7.16 (m, 1H), 7.30 (d, J=8.8 Hz, 2H), 7.41-7.45 (m, 3H), 7.53 (d, J=8.8 Hz, 2H), 7.64 (d, J=8.8 Hz, 2H), 7.87 (s, 1H).

### Example 8

### Synthesis of 4-(benzo[b]thiophen-2-yl)-1,3-bis(4-bromophenyl)-4-hydroxyimidazolidin-2-one

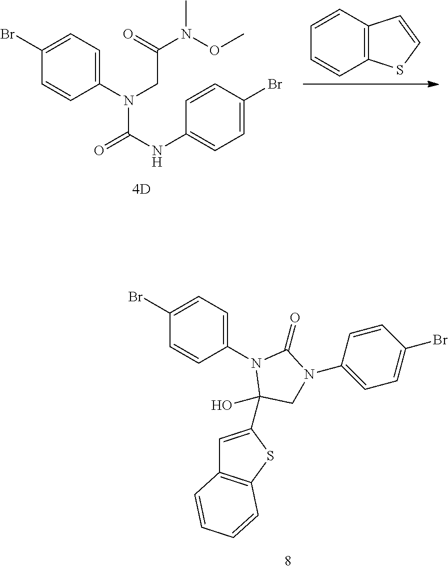

Compound 8 was synthesized by employing the procedure described for Compound 5 using benzo[b]thiophene in lieu of 2-bromo-5-chlorothiophene. LC-MS (ESI) m/z: 545 [M+H]⁺; ¹H-NMR (CDCl₃, 400 MHz): δ (ppm) 4.11 (s, 2H), 5.34 (brs, 1H), 7.18-7.36 (m, 11H), 7.64-7.73 (m, 2H).

### Example 9

### Synthesis of 1,3-bis(4-bromophenyl)-4-hydroxy-4-(pyridin-2-yl)imidazolidin-2-one

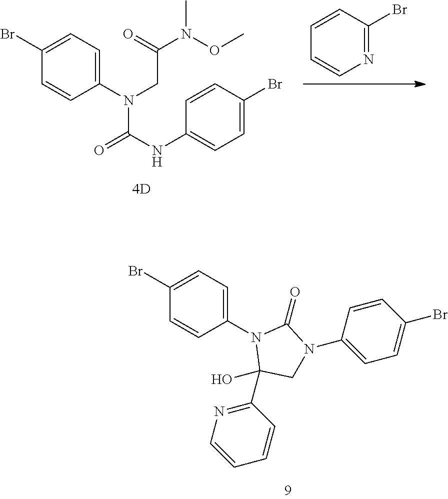

Compound 9 was synthesized by employing the procedure described for Compound 5 using 2-bromopyridine in lieu of 2-bromo-5-chlorothiophene. LC-MS (ESI) m/z: 470 [M-OH]⁺; ¹H-NMR (CDCl₃, 400 MHz): δ (ppm) 4.12-4.18 (m, 2H), 6.98-6.99 (m, 1H), 7.13-7.15 (m, 2H), 7.30-7.34 (m, 3H), 7.46-7.56 (m, 5H), 7.77-7.81 (m, 1H), 8.48-8.49 (m, 1H).

### Example 10

### Synthesis of 4-(benzo[d]thiazol-2-yl)-1,3-bis(4-bromophenyl)-4-hydroxyimidazolidin-2-one

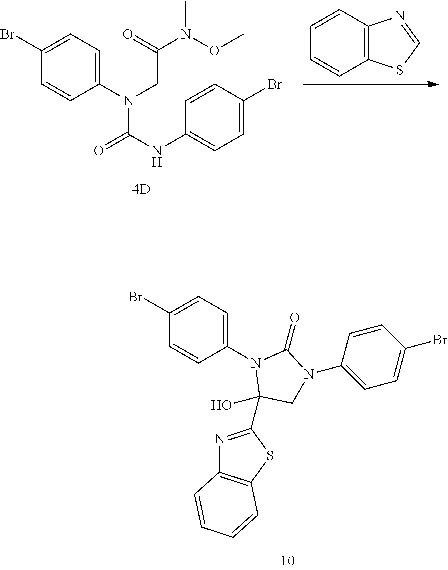

Compound 10 was synthesized by employing the procedure described for Compound 5 using benzo[d]thiazole in lieu of 2-bromo-5-chlorothiophene. LC-MS (ESI) m/z: 546 [M+H]⁺; ¹H-NMR (CDCl₃, 400 MHz): δ (ppm) 4.24 (d, J=10 Hz, 1H), 4.45 (d, J=10 Hz, 1H), 5.54 (brs, 1H), 7.26-7.38 (m, 4H), 7.42-7.53 (m, 6H), 7.86-7.99 (m, 2H).

### Example 11

### Synthesis of 1,3-bis(4-fluorophenyl)-4-hydroxy-4-phenylimidazolidin-2-one

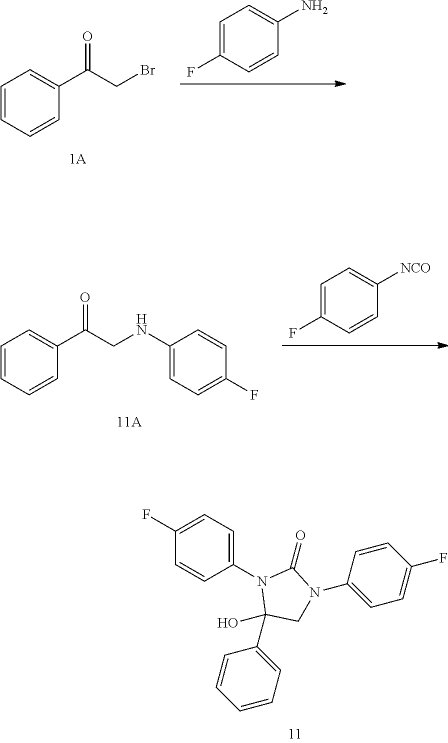

Compounds 11A and 11 were synthesized by employing the procedures described for Compounds 1B and 1 using 4-fluoroaniline, 1-fluoro-4-isocyanatobenzene, and Compound 11A in lieu of 4-bromoaniline, 1-bromo-4-isocyanatobenzene, and Compound 1B. Compound 11A: LC-MS (ESI) m/z: 230 [M+H]⁺. Compound 11: LC-MS (ESI) m/z: 367 [M+H]⁺; ¹H-NMR (DMSO-d₆, 400 MHz): δ (ppm) 4.05 (d, J=10.6 Hz, 1H), 4.16 (d, J=10.5 Hz, 1H), 7.04 (t, J=8.9 Hz, 2H), 7.15-7.42 (m, 7H), 7.52-7.62 (m, 3H), 7.64-7.77 (m, 2H).

### Example 12

### Synthesis of 1,3-bis(4-chlorophenyl)-4-hydroxy-4-phenylimidazolidin-2-one

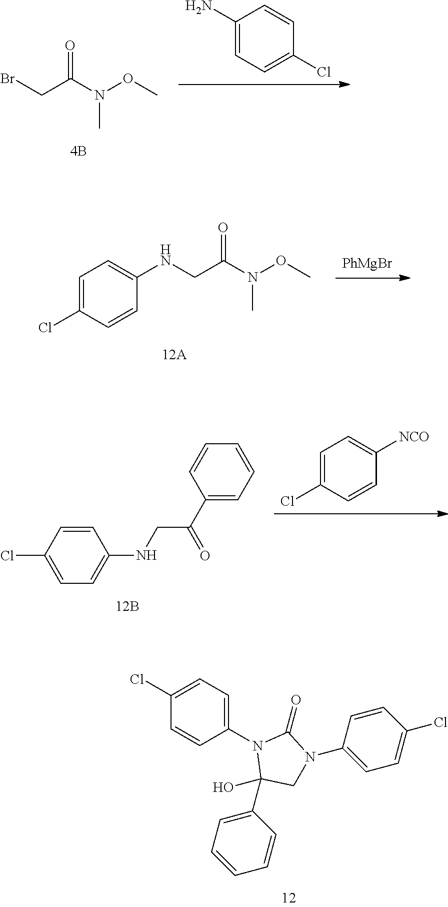

Compound 12A was synthesized by employing the procedure described for Compound 4C using 4-chloroaniline in lieu of 4-bromoaniline. LC-MS (ESI) m/z: 229 [M+H]⁺. ¹H-NMR (CDCl₃, 400 MHz): δ (ppm) 3.25 (s, 3H), 3.76 (s, 3H), 3.99 (s, 2H), 7.58 (d, J=9.2 Hz, 2H), 7.13 (d, J=8.8 Hz, 2H).

To a solution of PhMgBr (8.77 mL, 8.77 mmol) in THF (20 mL) was added a solution of Compound 12A (500 mg, 2.2 mmol) in THF (4 mL) at 0° C. The mixture was stirred at 0° C. for 2 hours, quenched with water (50 mL), and extracted with ethyl acetate (50 mL×2). The combined organic layers was washed with brine (50 mL), dried over Na₂SO₄, filtered, and concentrated under reduced pressure. The residue was slurried with Et₂O (10 mL) and filtered to afford Compound 12B: LC-MS (ESI) m/z: 246 [M+H]⁺.

A mixture of Compound 12B (100 mg, 0.41 mmol), 1-chloro-4-isocyanatobenzene (125 mg, 0.82 mmol), and DIPEA (158 mg, 1.22 mmol) in toluene (10 mL) was stirred at reflux for 3 hours. The mixture was cooled down to room temperature and filtered. The filtrate was concentrated under reduced pressure to give a crude product, which was purified with preparative HPLC to furnish Compound 12: LC-MS (ESI) m/z: 399 [M+H]⁺. ¹H-NMR (Acetone-d₆, 400 MHz): δ (ppm) 4.15 (d, J=10.8 Hz, 1H), 4.24 (d, J=10.8 Hz, 1H), 6.70 (s, 1H), 7.21 (d, J=8.8 Hz, 2H), 7.28-7.40 (m, 5H), 7.50 (d, J=8.8 Hz, 2H), 7.69-7.74 (m, 4H).

### Example 13

### Synthesis of 1,3-bis(4-bromophenyl)-4-hydroxy-5-methyl-4-phenylimidazolidin-2-one, (5S)-1,3-bis(4-bromophenyl)-4-hydroxy-5-methyl-4-phenylimidazolidin-2-one, and (5R)-1,3-bis(4-bromophenyl)-4-hydroxy-5-methyl-4-phenylimidazolidin-2-one

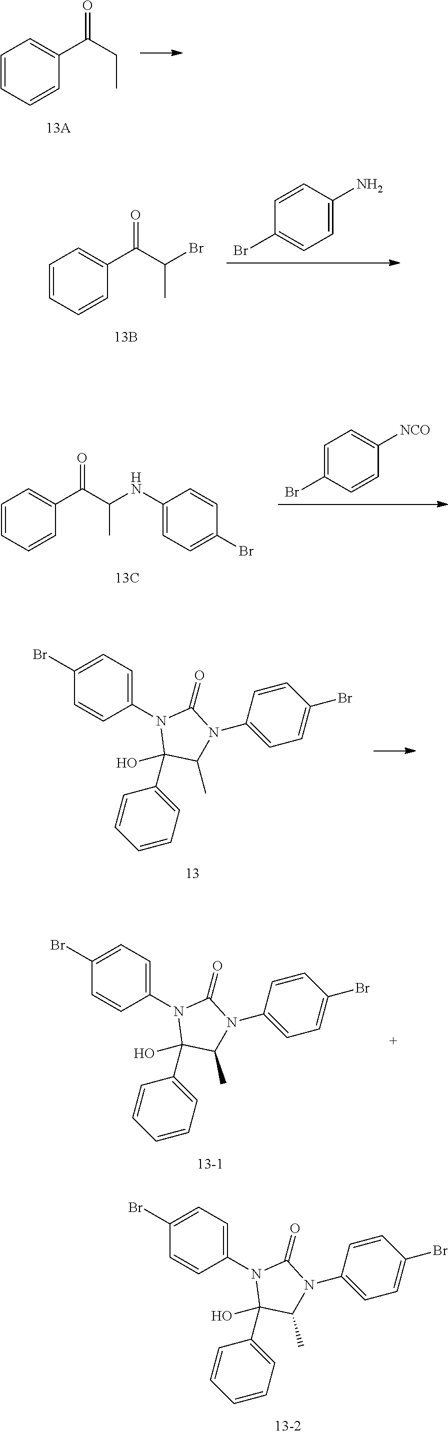

To a solution of Compound 13A (5.36 g, 40 mmol) in Et₂O (5 mL) was dropped neat Br₂ (2 mL, 40 mmol) at 0° C. It was stirred at 25° C. for 15 hours and quenched with a saturated Na₂S₂O₄ solution. It was stirred for 20 minutes and extracted with ethyl acetate (20 mL×3). The combined organic layers was washed with brine (40 mL), dried over anhydrous sodium sulfate, filtered, and concentrated. The crude product was purified with flash column chromatography on silica gel (ethyl acetate in petroleum ether, from 0% to 10% v/v) to yield Compound 13B: LC-MS (ESI) m/z: non-ionizable compound under routine conditions used.

To a solution of 4-bromoaniline (1.17 g, 5.5 mmol) and Et₃N (1.4 mL, 10 mmol) in THF (14 mL) was added Compound 13B (860 mg, 5 mmol). The mixture was refluxed for 48 hours and cooled down to room temperature. The precipitated white solid was filtered off and the filtrate was concentrated and purified with flash column chromatography on silica gel (ethyl acetate in petroleum ether, 10% v/v) to furnish Compound 13C: LC-MS (ESI) m/z: 304 [M+H]⁺; ¹H-NMR (CDCl₃, 400 MHz): δ (ppm) 1.48 (d, J=6.8 Hz, 3H), 4.77 (s, 1H), 5.06-5.13 (m, 1H), 6.55 (d, J=8.8 Hz, 2H), 7.27 (d, J=8.8 Hz, 2H), 7.53 (t, J=7.6 Hz, 2H), 7.62-7.64 (m, 1H), 8.02 (d, J=7.6 Hz, 2H).

Compound 13 was synthesized by employing the procedure described for Compound 12 using Compound 13C and 1-bromo-4-isocyanatobenzene in lieu of Compound 12B and 1-chloro-4-isocyanatobenzene. LC-MS: (ESI) m/z: 501 [M+H]⁺; ¹H-NMR (Acetone-d₆, 400 MHz): δ (ppm) 1.27 (d, J=4.8 Hz, 3H), 4.45 (m, 1H), 6.39 (s, 1H), 7.29-7.40 (m, 7H), 7.47 (d, J=5.2 Hz, 2H), 7.56 (d, J=5.2 Hz, 2H), 7.68 (d, J=5.2 Hz, 2H).

Compound 13 was separated by chiral HPLC to give Compound 13-1 and Compound 13-2. Compound 13-1: LC-MS (ESI) m/z: 501 [M+H]⁺; ¹H-NMR ((CD₃)₂CO, 400 MHz) δ (ppm) 1.26 (d, J=5.2 Hz, 3H), 4.42-4.46 (m, 1H), 6.35 (s, 1H), 7.27-7.39 (m, 7H), 7.46 (d, J=7.2 Hz, 2H), 7.56 (d, J=7.2 Hz, 2H), 7.67 (d, J=6 Hz, 2H). Chiral separation condition: MeOH contained 0.2% NH₄OH; IC (4.6×250 mm 5 μm); retention time: 1.42 minutes. Compound 13-2: LC-MS (ESI) m/z: 501 [M+H]⁺; ¹H-NMR ((CD₃)₂CO, 400 MHz) δ (ppm) 1.26 (d, J=5.2 Hz, 3H), 4.42-4.46 (m, 1H), 6.35 (s, 1H), 7.27-7.39 (m, 7H), 7.46 (d, J=7.2 Hz, 2H), 7.55 (d, J=7.2 Hz, 2H), 7.67 (d, J=6 Hz, 2H). Chiral separation condition: MeOH contained 0.2% NH₄OH; IC (4.6×250 mm 5 μm); retention time: 3.42 minutes.

### Example 14

### Synthesis of 1,3-bis(4-bromophenyl)-4-hydroxy-4-(o-tolyl)imidazolidin-2-one

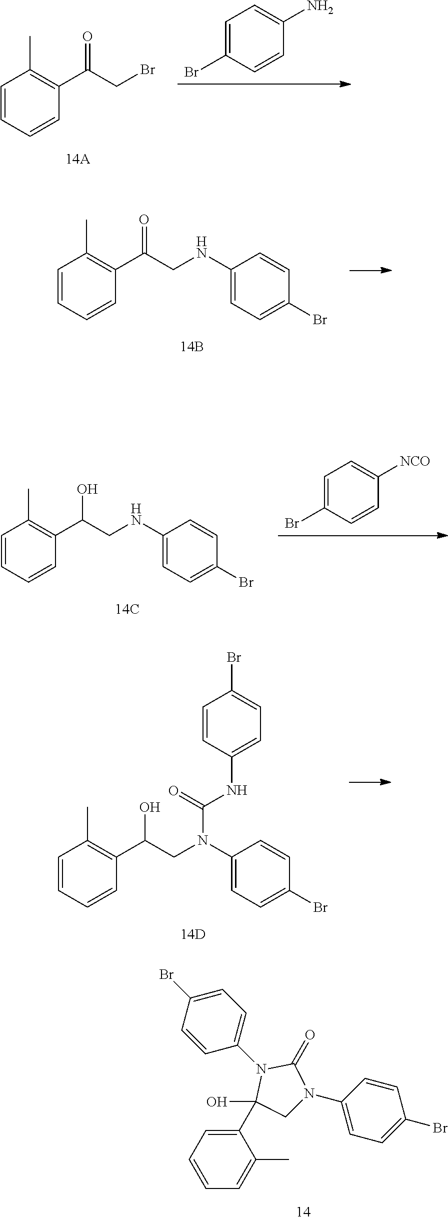

Compound 14B was synthesized by employing the procedure described for Compound 1B using Compound 14A in lieu of Compound 1A: LC-MS (ESI) m/z: 304 [M+H]⁺.

To a mixture of Compound 14B (150 mg, 0.49 mmol) in ethanol (5 mL) was added sodium boronhydride (54 mg, 1.47 mmol) in several small portions. The mixture was stirred at room temperature for 2 hours, quenched with a diluted HCl solution (0.5 M, 5 mL), and extracted with dichloromethane (20 mL×3). The combined organic layers was washed with water (50 mL×2) and brine (50 mL), dried over anhydrous sodium sulfate, filtered, and concentrated under reduced pressure to give Compound 14C: LC-MS (ESI) m/z: 306 [M+H]⁺.

Compound 14D was synthesized, by employing the procedure described for Compound 1 using Compound 14C in lieu of Compound 1B: LC-MS (ESI) m/z: 503 [M+H]⁺; ¹H-NMR (CDCl₃, 400 MHz): δ (ppm) 2.12 (s, 3H), 3.74-3.78 (m, 1H), 3.96-4.02 (m, 1H), 4.29 (d, J=8.4, 1H), 6.26 (s, 1H), 7.05-7.09 (m, 3H), 7.15-7.25 (m, 4H), 7.36-7.40 (m, 2H), 7.56-7.61 (m, 3H).

To a stirred solution of Compound 14D (50 mg, 0.1 mmol) in dichloromethane (5 mL) was added sodium bicarbonate (25 mg, 0.3 mmol) and Dess-Martin periodinane (42 mg, 0.1 mmol) at 0° C. The resulting mixture was stirred at room temperature for 2 hours, poured into a mixture of saturated Na₂S₂O₃ solution (10 mL) and saturated NaHCO₃ solution (10 mL), stirred vigorously for 30 minutes, and filtered. The filtrate was extracted with dichloromethane (20 mL×2). The combined organic layers was washed with water (100 mL×2) and brine (200 mL), dried over anhydrous sodium sulfate, filtered, and concentrated under reduced pressure to give a crude product, which was purified with preparative HPLC to afford Compound 14: LC-MS (ESI) m/z: 501 [M+H]⁺; ¹H-NMR (DMSO-d₆, 400 MHz): δ (ppm) 2.19 (s, 3H), 3.98 (d, J=9.2 Hz, 1H), 4.22 (d, J=9.2, 1H), 7.10-7.12 (m, 1H), 7.18-7.32 (m, 2H), 7.37-7.44 (m, 4H), 7.54-7.57 (m, 2H), 7.68-7.73 (m, 3H), 7.85 (d, J=4.0 Hz, 1H).

### Example 15

### Synthesis of 4-hydroxy-4-phenyl-1,3-bis(4-(trifluoromethyl)phenyl)imidazolidin-2-one

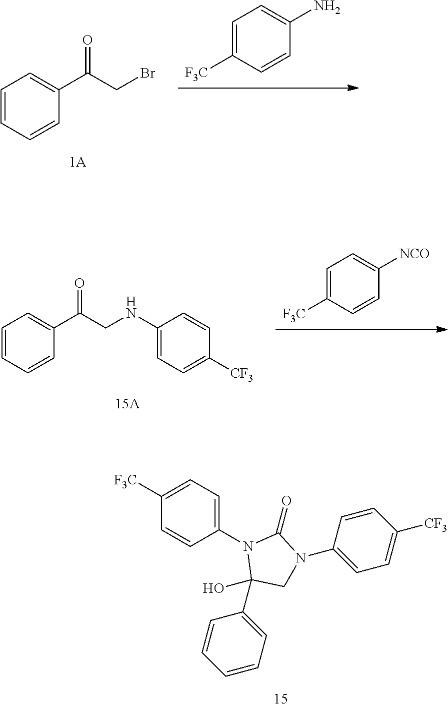

Compounds 15A and 15 were synthesized by employing the procedures described for Compounds 1B and 1 using 4-(trifluoromethyl)benzenamine, 1-isocyanato-4-(trifluoromethyl)benzene, and Compound 15A in lieu of 1-bromoaniline, 1-bromo-4-isocyanatobenzene, and Compound 1B. Compound 15A: LC-MS (ESI) m/z: 280 [M+H]⁺. Compound 15: LC-MS (ESI) m/z: 467 [M+H]⁺; ¹H-NMR (DMSO-d₆, 400 MHz): δ (ppm) 4.18 (q, J=10.7 Hz, 2H), 7.21-7.39 (m, 3H), 7.53-7.70 (m, 6H), 7.74 (d, J=8.8 Hz, 2H), 7.83-8.01 (m, 3H); ¹⁹F-NMR (376 MHz, DMSO-d₆): δ (ppm) −60.14 (s), −60.61 (s).

### Example 16

### Synthesis of 1,3-bis(4-bromophenyl)-4-hydroxy-4-(m-tolyl)imidazolidin-2-one

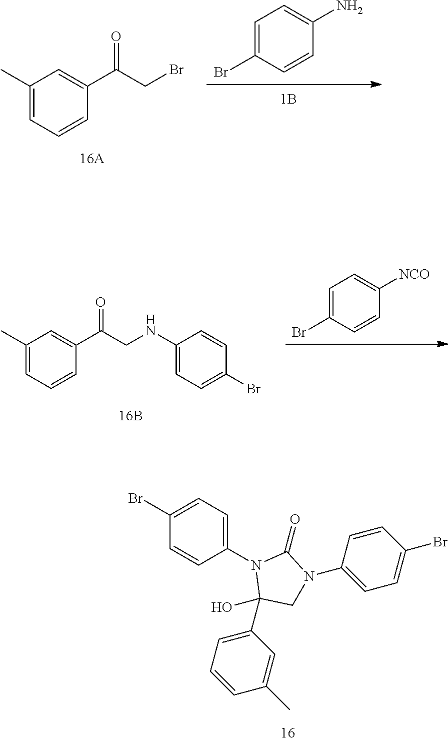

Compounds 16B and 16 were synthesized by employing the procedures described for Compounds 1B and 12 using Compounds 16A, 16B, and 1-bromo-4-isocyanatobenzene in lieu of Compounds 1A, 12B, and 1-chloro-4-isocyanatobenzene. Compound 16B: LC-MS (ESI) m/z: 304 [M+H]⁺; ¹H-NMR (DMSO-d₆, 400 MHz): δ (ppm) 2.40 (s, 3H), 4.65-4.66 (d, J=5.6 Hz, 2H), 6.11 (t, J=5.2 Hz, 1H), 6.65-6.67 (d, J=8.4 Hz, 2H), 7.20-7.22 (d, J=8.8 Hz, 2H), 7.42-7.50 (m, 2H), 7.85-7.89 (m, 2H). Compound 16: LC-MS (ESI) m/z: 501 [M+H]⁺; ¹H-NMR (DMSO-d₆, 400 MHz): δ (ppm) 2.26 (s, 3H), 4.00-4.12 (m, 2H), 7.05-7.07 (m, 1H), 7.17-7.22 (m, 1H), 7.35-7.41 (m, 5H), 7.53-7.55 (m, 2H), 7.64-7.67 (m, 3H).

### Example 17

### Synthesis of 4-hydroxy-4-phenyl-1,3-di-p-tolylimidazolidin-2-one

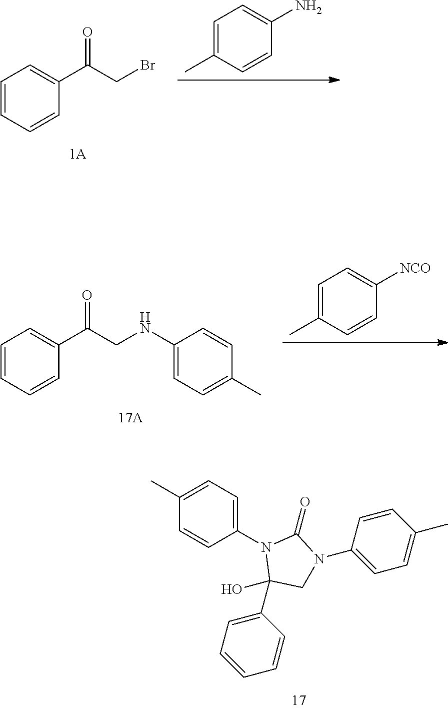

Compounds 17A and 17 were synthesized by employing the procedures described for Compounds 1B and 1 using p-toluidine, 1-isocyanato-4-methylbenzene, and Compound 17A in lieu of 4-bromoaniline, 1-bromo-4-isocyanatobenzene, and 1B. Compound 17A: LC-MS (ESI) m/z: 226 [M+H]⁺. Compound 17: LC-MS (ESI) m/z: 359 [M+H]⁺; ¹H-NMR (CD₃OD, 400 MHz): δ (ppm) 2.19 (s, 3H), 2.29 (s, 3H), 4.85 (d, J=10.8 Hz, 1H), 4.16 (d, J=10.8 Hz, 1H), 6.47 (s, 1H), 6.97 (d, J=4.0 Hz, 2H), 7.16 (d, J=8.4 Hz, 2H), 7.24-7.29 (m, 1H), 7.30-7.35 (m, 4H), 7.58 (d, J=8.8 Hz, 2H), 7.67-7.69 (m, 2H).

### Example 18

### Synthesis of 1,3-bis(4-bromophenyl)-1,3,4,5,6,7-hexahydro-2H-benzo[d]imidazol-2-one

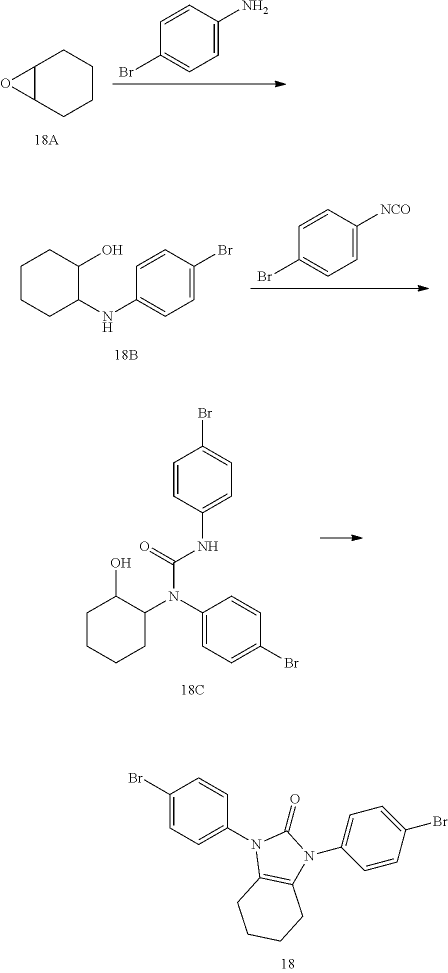

A mixture of Compound 18A (10 mmol) and 4-bromoaniline (1.71 g, 10 mmol) in methanol (100 mL) was heated to reflux overnight. The reaction mixture was concentrated under reduced pressure. The crude product was purified with flash column chromatography on silica gel (ethyl acetate in petroleum, 20% v/v) to furnish Compound 18B. LC-MS (ESI) m/z: 270 [M+H]⁺; ¹H-NMR (CDCl₃, 400 MHz): δ (ppm) 1.00-1.10 (m, 1H), 1.25-1.45 (m, 3H), 1.71-1.80 (m, 2H), 2.05-2.13 (m, 2H), 2.55 (s, 1H), 3.06-3.12 (m, 1H), 3.33-3.40 (m, 2H), 6.57-6.61 (m, 2H), 7.23-7.27 (m, 2H).

Compound 18C was synthesized by employing the procedure described for Compound 1 using Compound 18B in lieu of Compound 1B. Compound 18C. LC-MS (ESI) m/z: 467 [M+H]⁺; ¹H-NMR (CDCl₃, 400 MHz): δ (ppm) 0.93-1.10 (m, 2H), 1.24-1.44 (m, 2H), 1.65-1.70 (m, 2H), 1.80-1.84 (m, 1H), 2.08-2.11 (m, 1H), 2.56-2.60 (m, 1H), 3.21-3.29 (m, 1H), 4.37-4.44 (m, 1H), 5.91 (s, 1H), 7.12-7.15 (m, 2H), 7.24-7.28 (m, 2H), 7.31-7.35 (m, 2H), 7.63-7.65 (m, 2H).

A solution of Compound 18C (0.08 mmol), NaHCO₃ (34 mg, 0.4 mmol), and Dess-Martin Periodinane (53 mg, 0.12 mmol) in dichloromethane (5 mL) was stirred at room temperature overnight. The reaction mixture was concentrated under reduced pressure and the residue was purified with flash column chromatography on silica gel (ethyl acetate in petroleum ether, from 0% to 20% v/v) to yield Compound 18. LC-MS (ESI) m/z: 447 [M+H]⁺; ¹H-NMR (CDCl₃, 400 MHz): δ (ppm) 1.82 (s, 4H), 2.34 (s, 4H), 7.28 (d, J=8.4 Hz, 4H), 7.57 (d, J=8.4 Hz, 4H).

### Example 19

### Synthesis of 1,3-bis(4-bromophenyl)-4-(4-chlorophenyl)-4-hydroxyimidazolidin-2-one

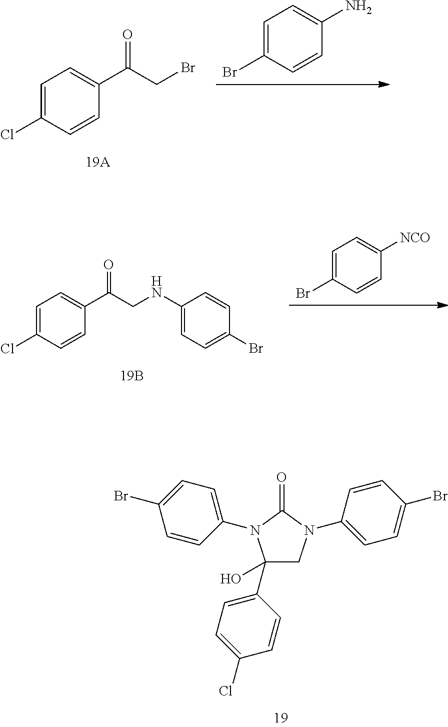

Compounds 19B and 19 were synthesized by employing the procedures described for Compounds 1B and 1 using Compounds 19A and 19B in lieu of Compounds 1A and 1B. Compound 19B: LC-MS (ESI) m/z: 324 [M+H]⁺; ¹H-NMR (DMSO-d₆, 400 MHz): δ (ppm) 4.67 (s, 2H), 6.12 (s, br, 1H), 6.63-6.67 (m, 2H), 7.19-7.22 (m, 2H), 7.62-7.65 (m, 2H), 8.06-8.08 (m, 2H). Compound 19: LC-MS (ESI) m/z: 521 [M+H]⁺; ¹H-NMR (DMSO-d₆, 400 MHz): δ (ppm) 4.04 (d, J=10.4 Hz, 1H), 4.13 (d, J=10.4 Hz, 1H), 7.30-7.45 (m, 6H), 7.54-7.66 (m, 6H), 7.80 (s, 1H).

### Example 20

### Synthesis of 3-(4-bromophenyl)-4-hydroxy-1,4-diphenylimidazolidin-2-one

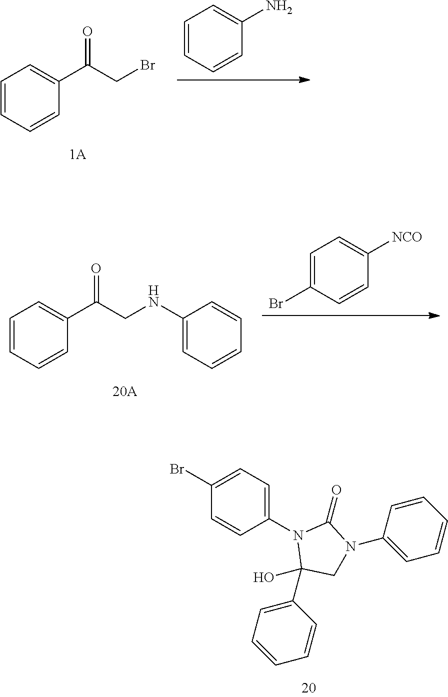

Compounds 20A and 20 were synthesized by employing the procedures described for Compounds 1B and 12 using aniline, Compound 20A, and 1-bromo-4-isocyanatobenzene in lieu of 4-bromoaniline, Compound 12B, and 1-chloro-4-isocyanatobenzene. Compound 20A: LC-MS (ESI) m/z: 212 [M+H]⁺. Compound 20: LC-MS (ESI) m/z: 409 [M+H]⁺. ¹H-NMR (Acetone-d₆, 400 MHz): δ (ppm) 4.21 (q, 2H), 6.70 (s, 1H), 7.07-7.09 (m, 1H), 7.33-7.38 (m, 7H), 7.46-7.48 (m, 2H), 7.69-7.71 (m, 4H).

### Example 21

### Synthesis of 4-hydroxy-1,3,4-triphenylimidazolidin-2-one

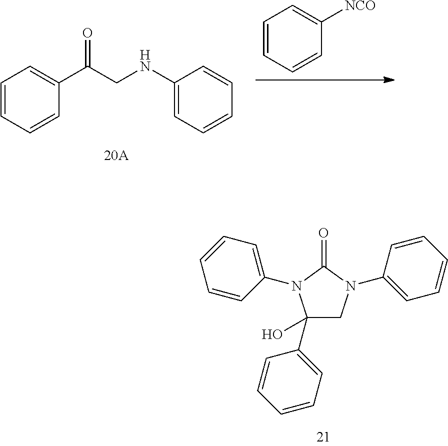

Compound 21 was synthesized by employing the procedure described for Compound 12 using isocyanatobenzene and Compound 20A in lieu of 1-chloro-4-isocyanatobenzene and Compound 12B: LC-MS (ESI) m/z: 331 [M+H]⁺. ¹H-NMR (CDCl₃, 400 MHz): δ (ppm) 4.07-4.16 (m, 2H), 5.63 (s, 1H), 7.07-7.12 (m, 2H), 7.18-7.22 (m, 2H), 7.29-7.46 (m, 7H), 7.56-7.60 (m, 4H).

### Example 22

### Synthesis of 1,3-bis(4-bromophenyl)-4,5-dipropyl-1,3-dihydro-2H-imidazol-2-one

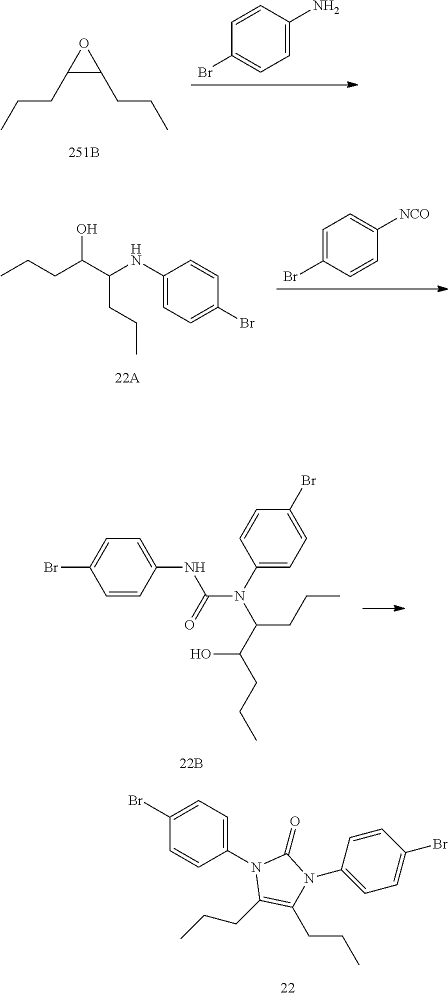

Compounds 22A, 22B, and 22 were synthesized by employing the procedures described for Compounds 251C, 1, and 18 using 4-bromoaniline, Compound 22A with 1,2-dichloroethane as the solvent and heated at 95° C., and Compound 22B in lieu of 4-chloroaniline, Compound 1B, and Compound 18C. Compound 22A. LC-MS (ESI) m/z: 300 [M+H]⁺; ¹H-NMR (CDCl₃, 400 MHz): δ (ppm) 0.88-0.94 (m, 6H), 1.30-1.63 (m, 8H), 1.97 (d, J=3.6 Hz, 1H), 3.22 (s, 1H), 3.61-3.67 (m, 2H), 6.49-6.52 (m, 2H), 7.20-7.24 (m, 2H). Compound 22B. LC-MS (ESI) m/z: 497 [M+H]⁺; ¹H-NMR (CDCl₃, 400 MHz): δ (ppm) 0.90-0.96 (m, 6H), 1.25-1.56 (m, 8H), 1.58-1.71 (m, 1H), 3.49 (s, 1H), 4.00 (br, 1H), 6.01 (s, 1H), 7.13-7.16 (m, 2H), 7.30-7.35 (m, 4H), 7.61-7.64 (m, 2H). Compound 22. LC-MS (ESI) m/z: 477 [M+H]⁺; ¹H-NMR (CD₃OD, 400 MHz): δ (ppm) 0.79 (t, J=7.6 Hz, 6H), 1.19-1.28 (m, 4H), 2.44 (t, J=7.6 Hz, 4H), 7.32 (d, J=8.4 Hz, 4H), 7.70 (d, J=8.4 Hz, 4H).

### Example 23

### Synthesis of 1,3-bis(4-bromophenyl)-4-hydroxy-4-(p-tolyl)imidazolidin-2-one

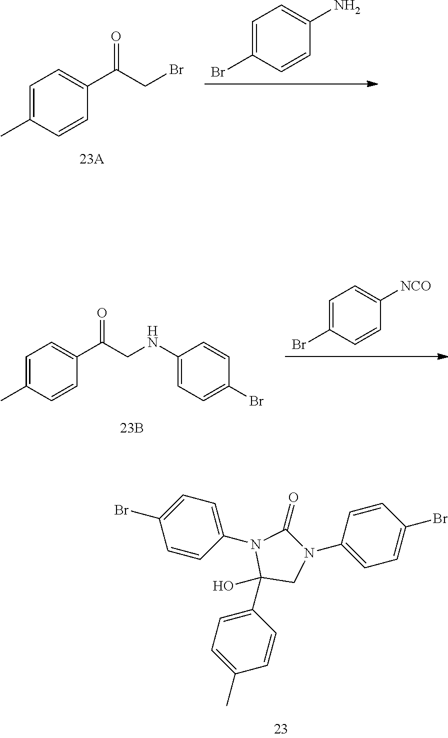

Compounds 23B and 23 were synthesized by employing the procedures described for Compounds 1B and 12 using Compounds 23A, 23B, and 1-bromo-4-isocyanatobenzene in lieu of Compounds 1A, 12B, and 1-chloro-4-isocyanatobenzene. Compound 23B: LC-MS (ESI) m/z: 304 [M+H]⁺; ¹H-NMR (CDCl₃, 400 MHz): δ (ppm) 2.44 (s, 3H), 4.54 (s, 2H), 6.58-6.61 (d, J=8.8 Hz, 2H), 7.30 (t, J=8.0 Hz, 4H), 7.89-7.91 (d, J=8.0 Hz, 2H). Compound 23: LC-MS (ESI) m/z: 501 [M+H]⁺; ¹H-NMR (DMSO-d₆, 400 MHz): δ (ppm) 2.24 (s, 3H), 4.00-4.10 (m, 2H), 7.10-7.12 (m, 2H), 7.32-7.40 (m, 4H), 7.42-7.45 (m, 2H), 7.53-7.55 (m, 2H), 7.61-7.66 (m, 3H).

### Example 24

### Synthesis of 1,3-bis(4-bromophenyl)-4-hydroxy-4-(naphthalen-2-yl)imidazolidin-2-one

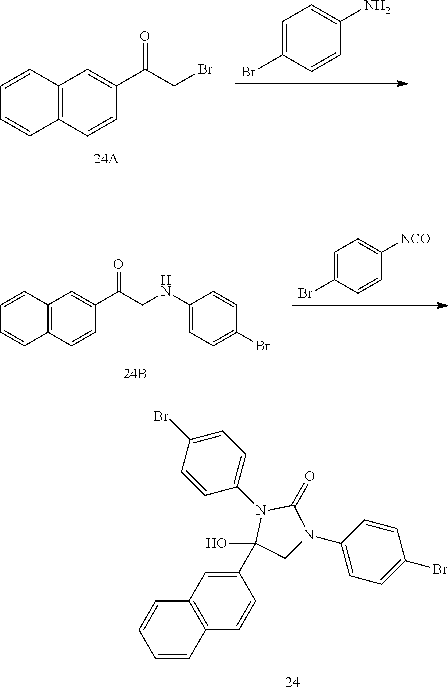

Compounds 24B and 24 were synthesized by employing the procedures described for Compounds 1B and 12 using Compounds 24A, 24B, and 1-bromo-4-isocyanatobenzene in lieu of Compounds 1A, 12, and 1-chloro-4-isocyanatobenzene. Compound 24B: LC-MS (ESI) m/z: 340 [M+H]⁺. Compound 24: LC-MS (ESI) m/z: 1095 [2M+Na]⁺. ¹H-NMR (Acetone-d₆, 400 MHz): δ (ppm) 4.22 (d, J=10.8 Hz, 1H), 4.36 (d, J=10.4 Hz, 1H), 6.87 (s, 1H), 7.32 (d, J=8.8 Hz, 2H), 7.49-7.55 (m, 6H), 7.70-7.74 (m, 3H), 7.85-7.88 (m, 2H), 7.94-7.95 (m, 1H), 8.33 (s, 1H).

### Example 25

### Synthesis of 1,3-bis(4-bromophenyl)-5-ethyl-4-hydroxy-4-phenylimidazolidin-2-one, (5R)-1,3-bis(4-bromophenyl)-5-ethyl-4-hydroxy-4-phenylimidazolidin-2-one, and (5S)-1,3-bis(4-bromophenyl)-5-ethyl-4-hydroxy-4-phenylimidazolidin-2-one

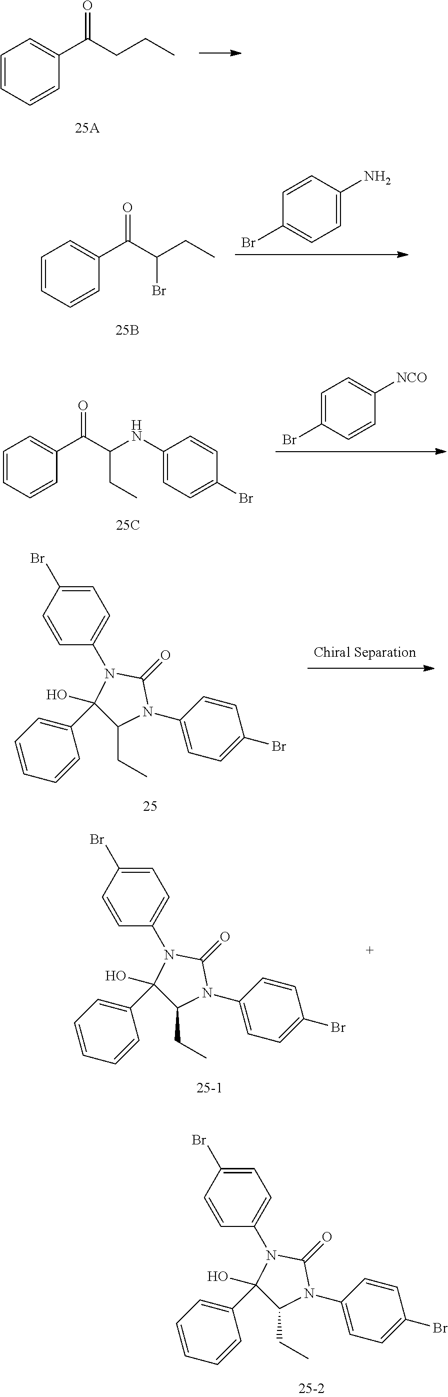

Compounds 25B, 25C, and 25 were synthesized by employing the procedures described for Compounds 13B, 13C, and 12 using Compounds 25A, 25B, 25C, and 1-bromo-4-isocyanatobenzene in lieu of Compounds 13A, 13B, 12B, and 1-chloro-4-isocyanatobenzene. Compound 25B. ¹H-NMR (CDCl₃, 400 MHz): δ (ppm) 1.09 (t, J=7.2 Hz, 3H), 2.12-2.26 (m, 2H), 5.06-5.09 (m, 1H), 7.47-7.51 (m, 2H), 7.57-7.61 (m, 1H), 8.01-8.03 (m, 2H). Compound 25C: LC-MS (ESI) m/z: 318 [M+H]⁺; ¹H-NMR (CDCl₃, 400 MHz): δ (ppm) 0.87 (t, J=7.2 Hz, 3H), 1.70-17.6 (m, 1H), 2.03-2.08 (m, 1H), 4.75-4.77 (m, 1H), 5.02-5.03 (m, 1H), 6.55-6.58 (m, 2H), 7.23-7.25 (m, 2H), 7.49-7.53 (m, 2H), 7.59-7.63 (m, 1H), 7.97-7.99 (m, 2H). Compound 25: LC-MS (ESI) m/z: 515 [M+H]⁺ was separated by chiral HPLC to give Compound 25-1 and Compound 25-2. Compound 25-1: LC-MS (ESI) m/z: 515 [M+H]⁺; ¹H-NMR (Acetone-d₆, 400 MHz): δ (ppm) 0.72 (t, J=6.4 Hz, 3H), 1.80-2.06 (m, 2H), 4.42-4.44 (m, 1H), 6.40 (br, 1H), 7.29-7.34 (m, 5H), 7.39-7.41 (m, 4H), 7.56-7.57 (m, 2H), 7.73-7.75 (m, 2H). Chiral separation condition: MeOH contained 0.2% NH₄OH; IC 250×4.6 mm 5 μm; retention time: 4.91 minutes. Compound 25-2: LC-MS (ESI) m/z: 515 [M+H]⁺; ¹H-NMR (Acetone-d₆, 400 MHz): δ (ppm) 0.72 (t, J=6.4 Hz, 3H), 1.80-2.08 (m, 2H), 4.42-4.44 (m, 1H), 6.40 (br, 1H), 7.26-7.35 (m, 5H), 7.39-7.41 (m, 4H), 7.55-7.57 (m, 2H), 7.73-7.75 (m, 2H). Chiral separation condition: MeOH contained 0.2% NH₄OH; IC 250×4.6 mm 5 μm; retention time: 1.82 minutes.

### Example 26

### Synthesis of 1,3-bis(4-bromophenyl)-4-hydroxy-4-phenylimidazolidine-2-thione

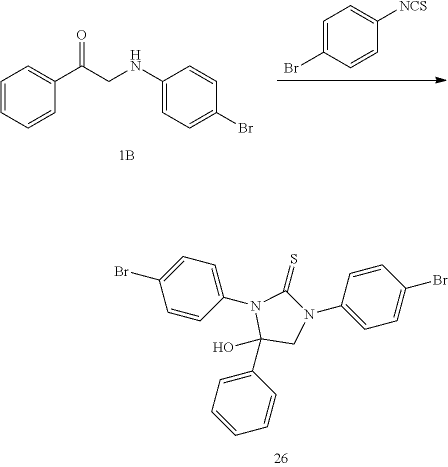

Compound 26 was synthesized by employing the procedure described for Compound 12 using Compound 1B and 1-bromo-4-isothiocyanatobenzene in lieu of Compound 12B and 1-chloro-4-isocyanatobenzene. LC-MS (ESI) m/z: 503 [M+H]⁺; ¹H-NMR (Acetone-d₆, 500 MHz): δ (ppm): 4.39 (d, J=12.0 Hz, 1H), 4.71 (d, J=11.5 Hz, 1H), 6.99 (s, 1H), 7.26-7.39 (m, 7H), 7.59 (d, J=9.0 Hz, 2H), 7.69 (d, J=7.5 Hz, 2H), 7.78 (d, J=9.0 Hz, 2H).

### Example 27

### Synthesis of 4-benzoyl-1,3-bis(4-chlorophenyl)-5-propyl-1,3-dihydro-2H-imidazol-2-one

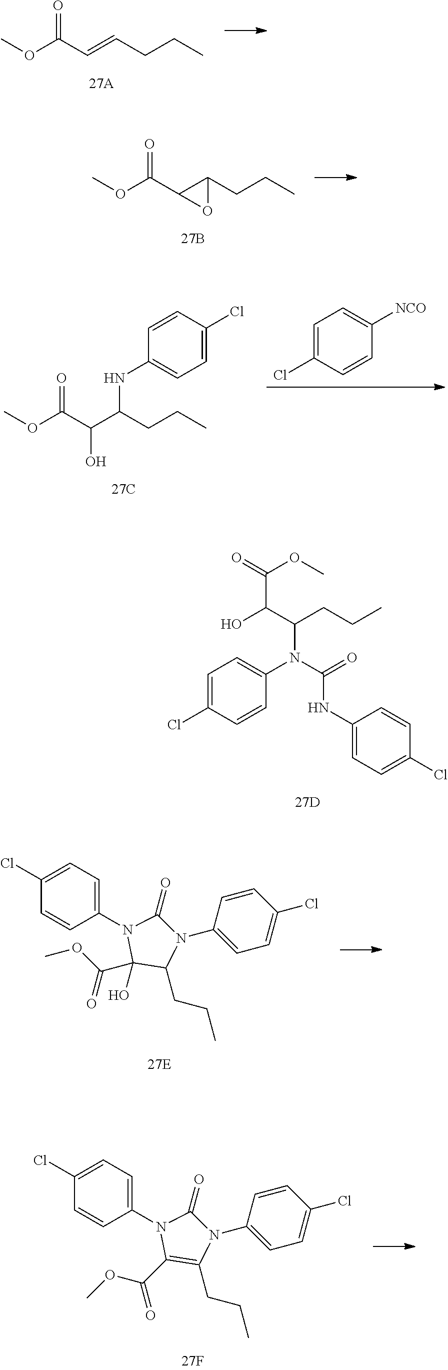

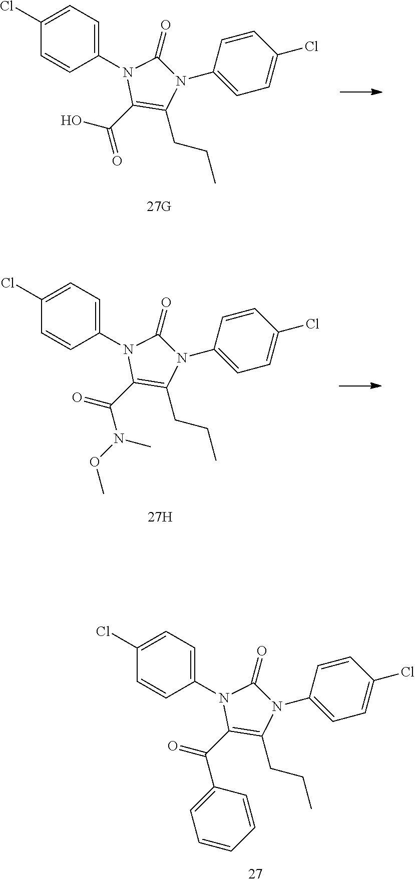 [SMILES: CCCc1c(C(=O)O)n(-c2ccc(Cl)cc2)c(=O)n1-c1ccc(Cl)cc1 ; CCCc1c(C(=O)N(C)OC)n(-c2ccc(Cl)cc2)c(=O)n1-c1ccc(Cl)cc1 ; CCCc1c(C(=O)c2ccccc2)n(-c2ccc(Cl)cc2)c(=O)n1-c1ccc(Cl)cc1]

Compounds 27B, 27C, 27D, 27E, and 27F were synthesized by employing the procedures described for Compounds 84B, 251C, 1, 14, and 255 using Compounds 27A, stirred at 45° C., 27B, 27C, 1-chloro-4-isocyanatobenzene, Compounds 27D and 27E in lieu of Compound 84A, stirred at room temperature, Compounds 251B, 1B, 1-bromo-4-isocyanatobenzene, Compounds 14D and 1. Compound 27B. LC-MS (ESI) m/z: non-ionizable compound under routine conditions used; ¹H-NMR (CDCl₃, 400 MHz): δ (ppm) 0.97 (t, J=7.2 Hz, 3H), 1.45-1.46 (m, 4H), 3.16 (t, J=6.4 Hz, 1H), 3.23 (t, J=2.4 Hz, 1H), 3.78 (s, 3H). Compound 27C. LC-MS (ESI) m/z: 272 [M+H]⁺; ¹H-NMR (CDCl₃, 400 MHz): δ (ppm) 0.82 (t, J=6.8 Hz, 3H), 1.22-1.26 (m, 2H), 1.39-1.45 (m, 2H), 2.77 (d, J=5.6 Hz, 1H), 3.65-3.74 (m, 2H), 3.76 (s, 3H), 4.29-4.32 (m, 1H), 6.53 (d, J=8.8 Hz, 2H), 7.05 (d, J=8.4 Hz, 2H). Compound 27D. LC-MS (ESI) m/z: 425 [M+H]⁺; ¹H-NMR (DMSO-d₆, 400 MHz): δ (ppm) 0.80 (t, J=7.2 Hz, 3H), 1.21-1.56 (m, 4H), 3.58 (s, 3H), 4.26-4.29 (m, 1H), 4.42 (s, 1H), 5.82 (d, J=6.4 Hz, 1H), 7.24 (d, J=9.2 Hz, 2H), 7.31 (d, J=8.4 Hz, 2H), 7.38 (d, J=8.4 Hz, 2H), 7.48 (d, J=8.8 Hz, 2H), 7.92 (s, 1H). Compound 27E. LC-MS (ESI) m/z: 423 [M+H]⁺. Compound 27F. LC-MS (ESI) m/z: 405 [M+H]⁺; ¹H-NMR (DMSO-d₆, 400 MHz): δ (ppm) 0.75 (t, J=7.6 Hz, 3H), 1.28-1.34 (m, 2H), 2.66 (t, J=7.6 Hz, 2H), 3.61 (s, 3H), 7.33-7.35 (m, 2H), 7.48-7.54 (m, 4H), 7.64 (d, J=8.4 Hz, 2H).

To a solution of Compound 27F (280 mg, 0.693 mmol) in EtOH (10 mL) was added a solution of NaOH (111 mg, 2.772 mmol) in water (1 mL). The mixture was stirred at 35° C. for 48 hours. To the reaction mixture was added a diluted aqueous HCl solution (3 N) until pH 3 and concentrated under reduced pressure. The residue was extracted with dichloromethane (15 mL×2). The combined organic layers was washed with brine (20 mL), dried over anhydrous sodium sulfate, filtered, and concentrated to give Compound 27G. LC-MS (ESI) m/z: 391 [M+H]⁺.

To a solution of Compound 27G (200 mg, 0.512 mmol) in dichloromethane (10 mL) at 0° C. was added N,O-dimethylhydroxylamine hydrochloride (300 mg, 3.07 mmol) and HATU (255 mg, 0.67 mmol), followed by N,N-diisopropylethylamine (529 mg, 4.1 mmol). The mixture was stirred at room temperature for 4 hours and heated at 35° C. overnight. The mixture was washed with water (20 mL) and brine (30 mL), dried over anhydrous sodium sulfate, filtered, and concentrated to give a crude product, which was purified with flash column chromatography on silica gel (ethyl acetate in petroleum ether, from 0% to 30% v/v) to yield Compound 27H. LC-MS (ESI) m/z: 434 [M+H]⁺; ¹H-NMR (CDCl₃, 400 MHz): δ (ppm) 0.79 (t, J=7.6 Hz, 3H), 1.23-1.33 (m, 2H), 2.54 (t, J=7.6 Hz, 2H), 3.11 (s, 3H), 3.47 (s, 3H), 7.29-7.39 (m, 6H), 7.48 (d, J=8.4 Hz, 2H).

To a solution of Compound 27H (115 mg, 0.265 mmol) in dry THF (10 mL) was dropped a solution of phenyllithium in di-n-butyl ether (1.9 M, 0.7 mL, 1.32 mmol) at −78° C. under nitrogen. The mixture was stirred at −78° C. under nitrogen for 1 hour, quenched with saturated aqueous NH₄Cl solution (2 mL), and extracted with ethyl acetate (15 mL×2). The combined extracts was washed with brine (10 mL), dried over anhydrous sodium sulfate, filtered, and concentrated under reduced pressure. The residue was purified with flash column chromatography on silica gel (ethyl acetate in petroleum ether, from 0% to 20% v/v) to yield Compound 27. LC-MS (ESI) m/z: 451 [M+H]⁺; ¹H-NMR (CD₃OD, 400 MHz): δ (ppm) 0.60 (t, J=7.6 Hz, 3H), 1.18-1.26 (m, 2H), 2.40 (t, J=7.6 Hz, 2H), 7.27-7.35 (m, 4H), 7.43-7.51 (m, 2H), 7.52-7.62 (m, 5H), 7.80 (d, J=7.2 Hz, 2H).

### Example 28

### Synthesis of 1-(4-bromophenyl)-4-hydroxy-3,4-diphenylimidazolidin-2-one

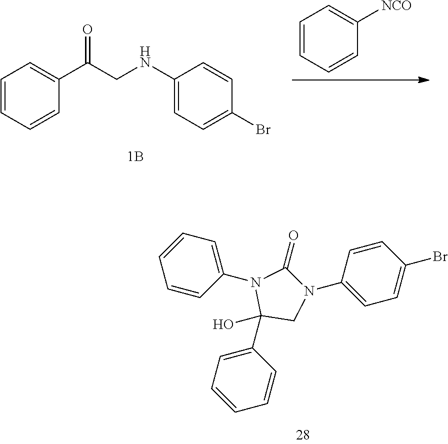

Compound 28 was synthesized by employing the procedure described for Compound 12 using Compound 1B and isocyanatobenzene in lieu of Compound 12B and 1-chloro-4-isocyanatobenzene. LC-MS (ESI) m/z: 409 [M+H]⁺; ¹H-NMR (Acetone-d₆, 500 MHz): δ (ppm): 4.14 (d, J=10.5 Hz, 1H), 4.22 (d, J=10.5 Hz, 1H), 6.62 (s, 1H), 7.03-7.05 (m, 1H), 7.16-7.19 (m, 2H), 7.26-7.26 (m, 1H), 7.31-7.34 (m, 2H), 7.46-7.52 (m, 4H), 7.68-7.70 (m, 4H).

### Example 29

### Synthesis of 1,3-bis(4-bromophenyl)-4-hydroxy-5-methyl-4-(m-tolyl)imidazolidin-2-one, (5S)-1,3-bis(4-bromophenyl)-4-hydroxy-5-methyl-4-(m-tolyl)imidazolidin-2-one, and (5R)-1,3-bis(4-bromophenyl)-4-hydroxy-5-methyl-4-(m-tolyl)imidazolidin-2-one

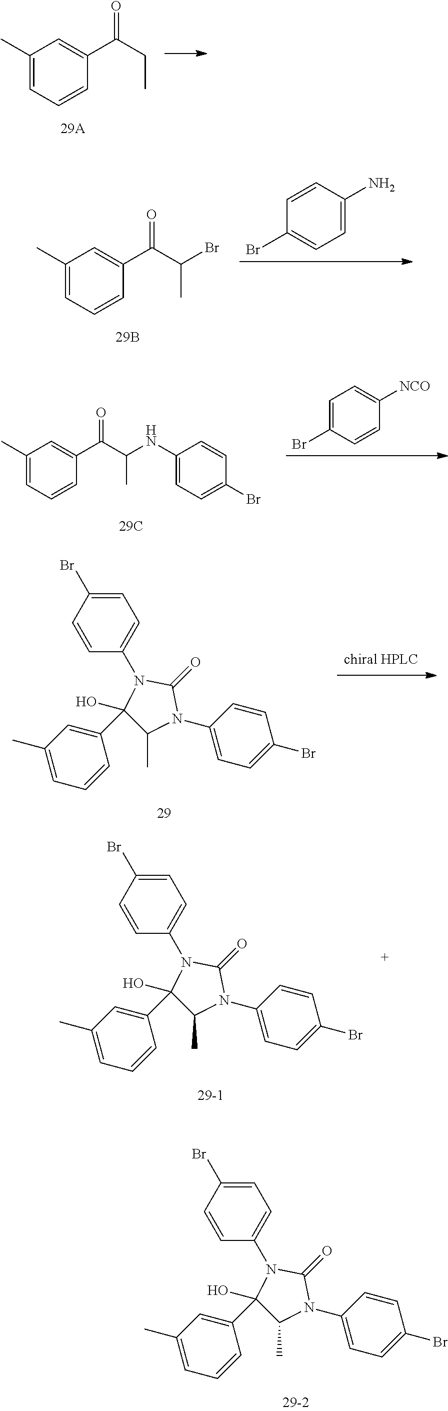

Compounds 29B, 29C, and 29 were synthesized by employing the procedures described for Compounds 13B, 13C, and 12 using Compounds 29A, 29B, 29C, and 1-bromo-4-isocyanatobenzene in lieu of Compounds 13A, 13B, 12B, and 1-chloro-4-isocyanatobenzene. Compound 29B: LC-MS (ESI) m/z: 227 [M+H]⁺. Compound 29C: LC-MS (ESI) m/z: 318 [M+H]⁺. Compound 29 was separated by chiral HPLC to furnish Compound 29-1 and Compound 29-2. Compound 29-1: LC-MS (ESI) m/z: 515.0 [M+H]⁺; ¹H-NMR (Acetone-d₆, 400 MHz): δ (ppm) 1.26 (d, J=6.4 Hz, 3H), 2.30 (s, 3H), 4.42-4.45 (m, 1H), 6.30 (s, 1H), 7.10 (d, J=7.2 Hz, 1H), 7.22 (t, J=7.2 Hz, 1H), 7.30-7.40 (m, 4H), 7.44-7.50 (m, 3H), 7.52-7.58 (m, 3H). Chiral separation condition: MeOH contained 0.2% NH₄OH; OJ-H (4.6*250 mm, 5 μm); retention time: 3.23 minutes. Compound 29-2: LC-MS (ESI) m/z: 515.0 [M+H]⁺; ¹H-NMR (Acetone-d₆, 400 MHz): δ (ppm) 1.26 (d, J=6.4 Hz, 3H), 2.30 (s, 3H), 4.42-4.45 (m, 1H), 6.30 (s, 1H), 7.10 (d, J=7.2 Hz, 1H), 7.22 (t, J=7.2 Hz, 1H), 7.30-7.40 (m, 4H), 7.44-7.50 (m, 3H), 7.52-7.58 (m, 3H). Chiral separation condition: MeOH contained 0.2% NH₄OH; OJ-H (4.6*250 mm, 5 μm); retention time: 3.78 minutes.

### Example 30

### Synthesis of 1,3-bis(4-bromophenyl)-4-hydroxy-4-(3-methoxyphenyl)imidazolidin-2-one

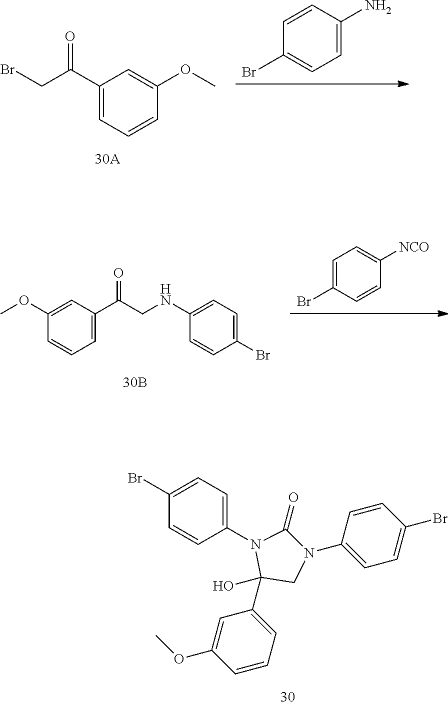

Compounds 30B and 30 were synthesized by employing the procedures described for Compounds 1B and 1C using Compounds 30A, 30B, and heated at 80° C. in lieu of Compounds 1A, 1B, and stirred at room temperature. Compound 30B: LC-MS (ESI) m/z: 320 [M+H]⁺. Compound 30: LC-MS (ESI) m/z: 517 [M+H]⁺; ¹H-NMR (DMSO-d₆, 400 MHz): δ (ppm) 3.69 (s, 3H), 4.02 (d, J=10.8 Hz, 1H), 4.12 (d, J=10.8 Hz, 1H), 6.80-6.82 (m, 1H), 7.1-7.14 (m, 2H), 7.20-7.24 (m, 1H), 7.33-7.35 (m, 2H), 7.39-7.41 (m, 2H), 7.53-7.55 (m, 2H), 7.63-7.68 (m, 3H).

### Example 31

### Synthesis of 1,3-bis(4-bromophenyl)-4-hydroxy-4-(3-(trifluoromethyl)phenyl)imidazolidin-2-one

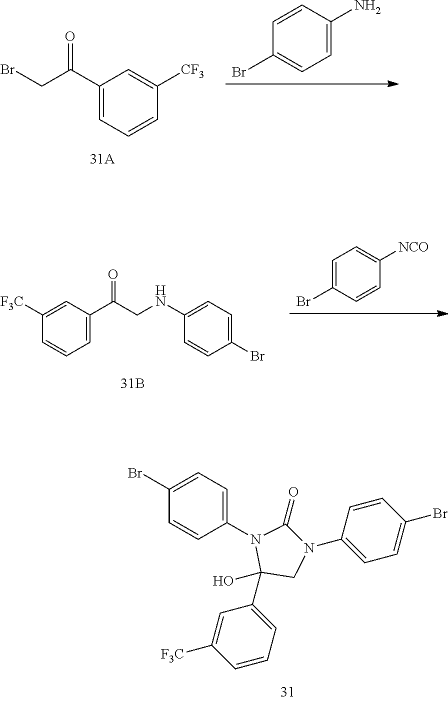

Compounds 31B and 31 were synthesized by employing the procedures described for Compounds 1B and 1C using Compounds 31A, 31B, and heated at 80° C. in lieu of Compounds 1A, 1B, and stirred at room temperature. Compound 31B: LC-MS (ESI) m/z: 358 [M+H]⁺. Compound 31: LC-MS (ESI) m/z: 555 [M+H]⁺; ¹H-NMR (DMSO-d₆, 400 MHz): δ (ppm) 4.07 (d, J=10.8 Hz, 1H), 4.19 (d, J=10.8 Hz, 1H), 7.29-7.32 (m, 2H), 7.39-7.42 (m, 2H), 7.54-7.57 (m, 3H), 7.63-7.66 (m, 3H), 7.88-7.90 (m, 1H), 7.96 (s, 2H).

### Example 32

### Synthesis of 1,3-bis(3,4-dichlorophenyl)-4-hydroxy-4-phenylimidazolidin-2-one

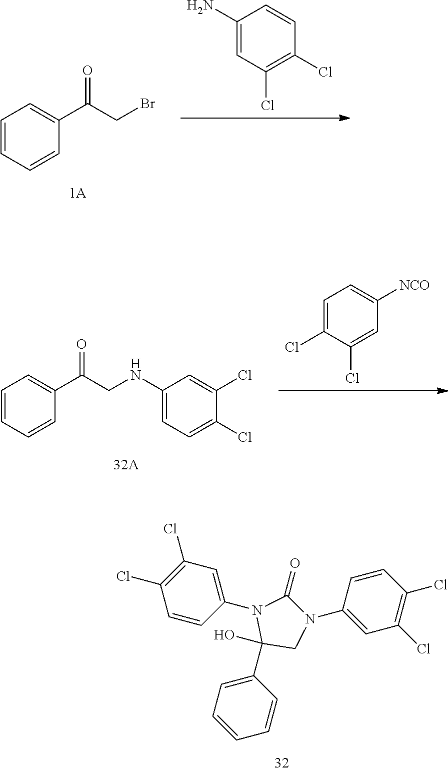

Compounds 32A and 32 were synthesized by employing the procedures described for Compounds 13C and 1 using Compounds 1A, 3,4-dichloroaniline, Compound 32A, and 1,2-dichloro-4-isocyanatobenzene in lieu of Compounds 13B, 4-bromoaniline, Compound 1B, and 1-bromo-4-isocyanatobenzene. Compound 32A: LC-MS (ESI) m/z: 280 [M+H]⁺. Compound 32: LC-MS: (ESI) m/z: 467 [M+H]⁺; ¹H-NMR (acetone-d₆, 400 MHz): δ (ppm) 4.22-4.29 (m, 2H), 6.88 (s, 1H), 7.30-7.32 (m, 1H), 7.35-7.39 (m, 3H), 7.47-7.49 (m, 1H), 7.55 (d, J=7.2 Hz, 1H), 7.62-7.64 (m, 1H), 7.71-7.73 (m, 2H), 7.77 (s, 1H), 8.05 (s, 1H).

### Example 33

### Synthesis of 1,3-bis(4-bromophenyl)-4-(3,5-dimethylphenyl)-4-hydroxyimidazolidin-2-one

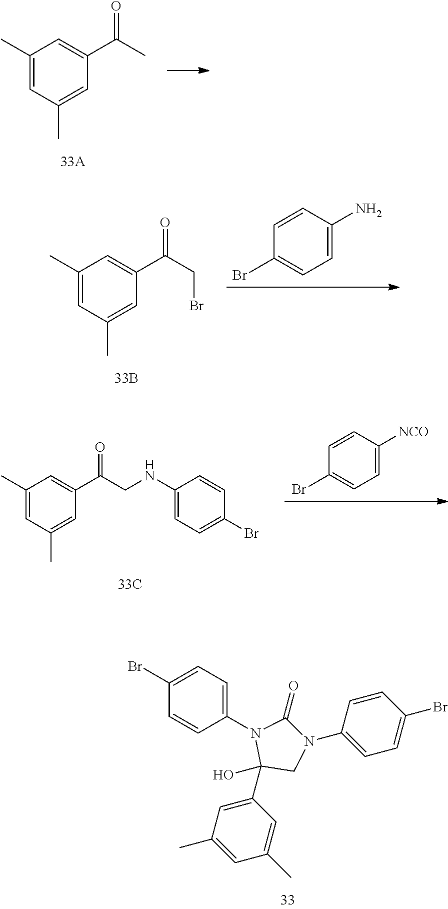

Compounds 33B, 33C, and 33 were synthesized by employing the procedures described for Compounds 13B, 1B, and 1 using Compounds 33A, 33B, and 33C in lieu of Compounds 13B, 1A, and 1B. Compound 33B: LC-MS (ESI) m/z: 227 [M+H]⁺; ¹H-NMR (CDCl₃, 400 MHz): δ (ppm) 2.38 (s, 6H), 4.45 (s, 2H), 7.25 (s, 1H), 7.59 (s, 2H). Compound 33C: LC-MS (ESI) m/z: 318 [M+H]⁺; ¹H-NMR (DMSO-d₆, 400 MHz): δ (ppm) 2.33 (s, 6H), 4.63 (d, J=5.2 Hz, 2H), 6.10 (t, J=5.2 Hz, 1H), 6.65 (d, J=8.8 Hz, 2H), 7.20 (d, J=9.2 Hz, 2H), 7.31 (s, 1H), 7.68 (s, 2H). Compound 33: LC-MS (ESI) m/z: 515 [M+H]⁺; ¹H-NMR (DMSO-d₆, 400 MHz): δ (ppm) 2.21 (s, 6H), 3.99-4.08 (m, 2H), 6.88 (s, 1H), 7.18 (s, 2H), 7.34 (d, J=9.2 Hz, 2H), 7.40 (d, J=8.8 Hz, 2H), 7.53 (d, J=9.2 Hz, 2H), 7.58 (s, 1H), 7.64 (d, J=8.8 Hz, 2H).

### Example 34

### Synthesis of 1,3-bis(4-chlorophenyl)-4-hydroxy-4-(m-tolyl)imidazolidin-2-one

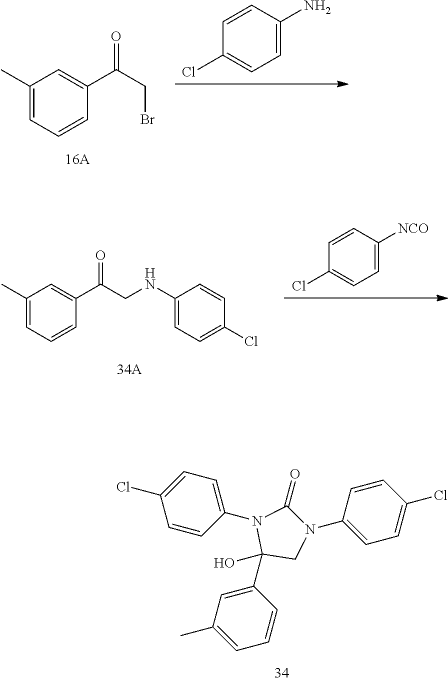

Compounds 34A and 34 were synthesized by employing the procedures described for Compounds 1B and 1 using Compounds 16A, 4-chloroanilline, 34A, and 1-chloro-4-isocyanatobenzene in lieu of Compounds 1A, 4-bromoanilline, 1B, and 1-bromo-4-isocyanatobenzene. Compound 34A: LC-MS (ESI) m/z: 260 [M+H]⁺. Compound 34: LC-MS: (ESI) m/z: 413 [M+H]⁺; ¹H-NMR (DMSO-d₆, 400 MHz): δ (ppm) 2.25 (s, 3H), 4.03 (q, 2H), 7.06 (d, J=7.6 Hz, 1H), 7.19 (dd, J=7.6, 7.6 Hz, 1H), 7.25-7.29 (m, 2H), 7.34 (d, J=7.6 Hz, 1H), 7.38-7.42 (m, 5H), 7.61 (s, 1H), 7.69-7.71 (m, 2H).

### Example 35

### Synthesis of 1,3-bis(4-bromophenyl)-4-(3-chlorophenyl)-4-hydroxyimidazolidin-2-one

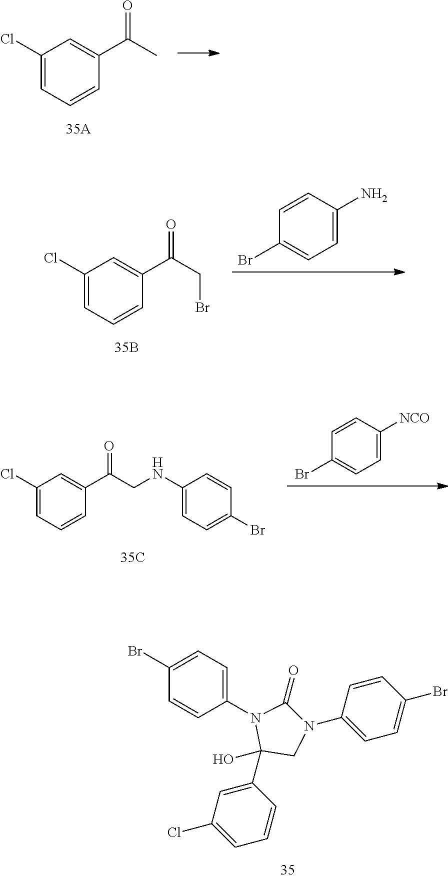

Compounds 35B, 35C, and 35 were synthesized by employing the procedures described for Compounds 13B, 1B, and 1 using Compounds 35A, 35B, and 35C in lieu of Compounds 13A, 1A and 1B. Compound 35B: LC-MS (ESI) m/z: non-ionizable compound under routine conditions used; ¹H-NMR (CDCl₃, 400 MHz): δ (ppm) 4.35 (s, 2H), 7.38 (t, J=8.0 Hz, 1H), 7.51 (d, J=8.4 Hz, 1H), 7.79 (d, J=8.4 Hz, 1H), 7.89 (s, 1H). Compound 35C: LC-MS (ESI) m/z: 324 [M+H]⁺; ¹H-NMR (DMSO-d₆, 400 MHz): δ (ppm) 4.70 (d, J=5.2 Hz, 2H), 6.16 (t, J=5.6 Hz, 1H), 6.66 (d, J=6.8 Hz, 2H), 7.20 (d, J=9.2 Hz, 2H), 7.60 (t, J=8.0 Hz, 1H), 7.74-7.77 (m, 1H), 7.99-8.02 (m, 1H), 8.09 (t, J=2.0 Hz, 1H). Compound 35: LC-MS (ESI) m/z: 521 [M+H]⁺; ¹H-NMR (CDCl₃, 400 MHz): δ (ppm) 3.84 (d, J=10.4 Hz, 1H), 3.99 (d, J=10.8 Hz, 1H), 5.45 (s, 1H), 7.10-7.26 (m, 9H), 7.31-7.33 (m, 2H), 7.49 (s, 1H).

### Example 36

### Synthesis of 1,3-bis(4-chlorophenyl)-4-hydroxy-5-methyl-4-(m-tolyl)imidazolidin-2-one, (5S)-1,3-bis(4-chlorophenyl)-4-hydroxy-5-methyl-4-(m-tolyl)imidazolidin-2-one, and (5R)-1,3-bis(4-chlorophenyl)-4-hydroxy-5-methyl-4-(m-tolyl)imidazolidin-2-one

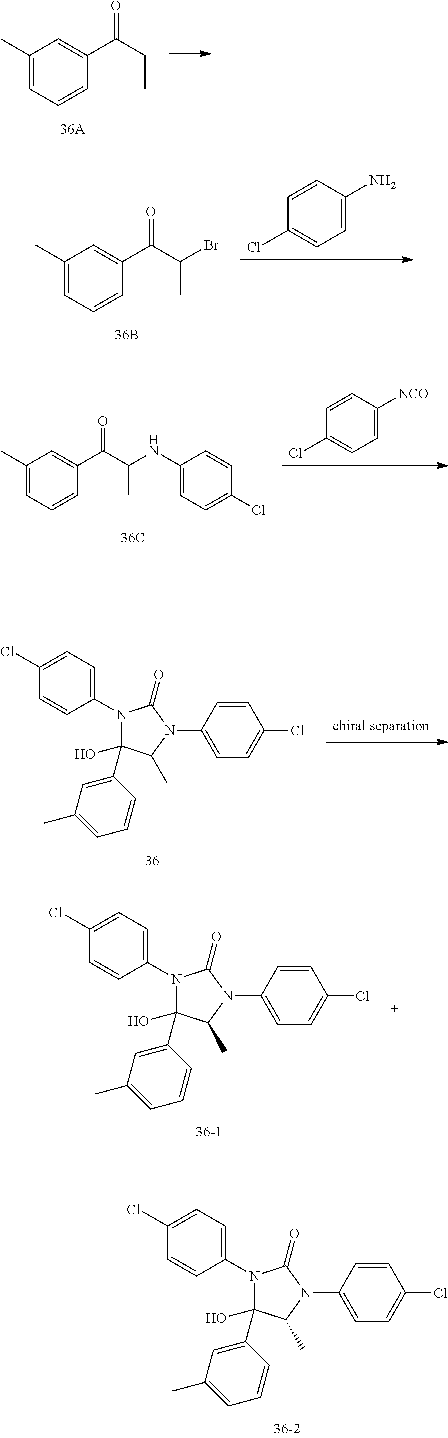

Compounds 36B, 36C, and 36 were synthesized by employing the procedures described for Compounds 13B, 13C, and 12 using Compounds 36A, 36B, 4-chloroanilline, and 36C in lieu of Compounds 13A, 13B, 4-bromoanilline, and 12B. Compound 36B: LC-MS (ESI) m/z: 227 [M+H]⁺. Compound 36C: LC-MS (ESI) m/z: 274 [M+H]⁺. Compound 36: LC-MS (ESI) m/z: 427 [M+H]⁺; ¹H-NMR (DMSO-d₆, 400 MHz): δ (ppm) 1.08 (d, J=6.4 Hz, 3H), 2.26 (s, 3H), 4.32-4.35 (m, 1H), 7.04-7.28 (m, 6H), 7.32-7.37 (m, 1H), 7.40-7.47 (m, 6H). Compound 36 was separated with chiral HPLC to give Compound 36-1 and Compound 36-2. Compound 36-1: LC-MS (ESI) m/z: 427 [M+H]⁺; ¹H-NMR (DMSO-d₆, 400 MHz): δ (ppm) 1.08 (d, J=6.4 Hz, 3H), 2.26 (s, 3H), 4.30-4.37 (m, 1H), 7.07 (d, J=6.8 Hz, 1H), 7.16-7.38 (m, 6H), 7.39-7.47 (m, 6H). Chiral separation condition: MeOH contained 0.2% Methanol ammonia; AS-H (4.6*250 mm, 5 μm); retention time: 2.64 minutes. Compound 36-2: LC-MS (ESI) m/z: 427 [M+H]⁺; ¹H-NMR (DMSO-d₆, 400 MHz): δ (ppm) 1.08 (d, J=6.4 Hz, 3H), 2.26 (s, 3H), 4.30-4.37 (m, 1H), 7.07 (d, J=6.8 Hz, 1H), 7.16-7.38 (m, 6H), 7.39-7.47 (m, 6H). Chiral separation condition: MeOH contained 0.2% Methanol ammonia; AS-H (4.6*250 mm, 5 μm); retention time: 3.32 minutes.

### Example 37

### Synthesis of 1,3-bis(4-chlorophenyl)-4-(3-(1-hydroxyethyl)phenyl)-5-methyl-1,3-dihydro-2H-imidazol-2-one

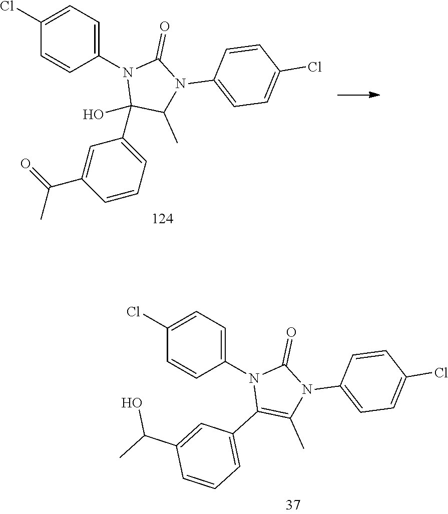 [SMILES: CC(=O)c1cccc(C2(O)C(C)N(c3ccc(Cl)cc3)C(=O)N2c2ccc(Cl)cc2)c1 ; Cc1c(-c2cccc(C(C)O)c2)n(-c2ccc(Cl)cc2)c(=O)n1-c1ccc(Cl)cc1]

To a solution of Compound 124 (30 mg, 0.066 mmol) in anhydrous methanol (5 mL) at 0° C. under nitrogen atmosphere was added sodium borohydride (5 mg, 0.132 mmol). The mixture was stirred at room temperature for 3 hours, quenched by addition of acetic acid (0.2 mL) dropwise, and concentrated under reduced pressure. The residue was purified with preparative HPLC to afford Compound 37. LC-MS (ESI) m/z: 439 [M+H]⁺; ¹H-NMR (acetine-d₆, 400 MHz): δ (ppm) 1.27 (d, J=6.8 Hz, 3H), 2.10 (s, 3H), 4.19 (d, J=3.2 Hz, 1H), 4.74-4.81 (m, 1H), 7.04-7.07 (m, 1H), 7.18-7.22 (m, 3H), 7.26-7.36 (m, 4H), 7.53-7.60 (m, 4H).

### Example 38

### Synthesis of 3-(4-bromophenyl)-1-(4-chlorophenyl)-4-hydroxy-4-phenylimidazolidin-2-one

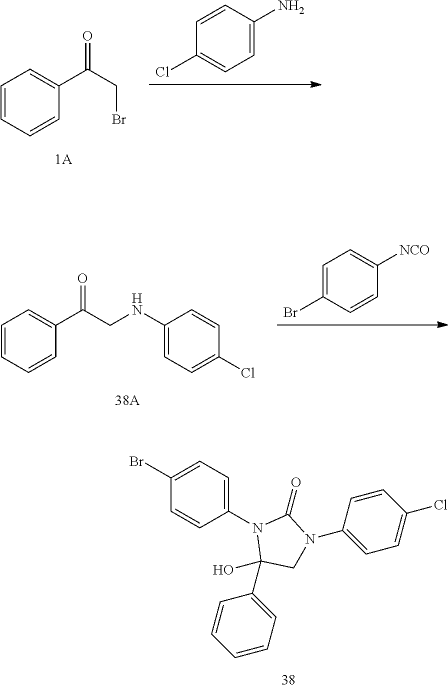

Compounds 38A and 38 were synthesized by employing the procedures described for Compounds 1B and 1 using 4-chloroaniline and Compounds 38B in lieu of 4-brommoaniline and 1B. Compound 38A: LC-MS (ESI) m/z: 246 [M+1]⁺. Compound 38: LC-MS (ESI) m/z: 443 [M+H]⁺; ¹H-NMR (DMSO-d₆, 400 MHz): δ (ppm) 4.04-4.15 (m, 2H), 7.23-7.43 (m, 9H), 7.57-7.58 (m, 2H), 7.68-7.72 (m, 3H).

### Example 39

### Synthesis of 4-(3-chlorophenyl)-1,3-bis(4-chlorophenyl)-4-hydroxyimidazolidin-2-one

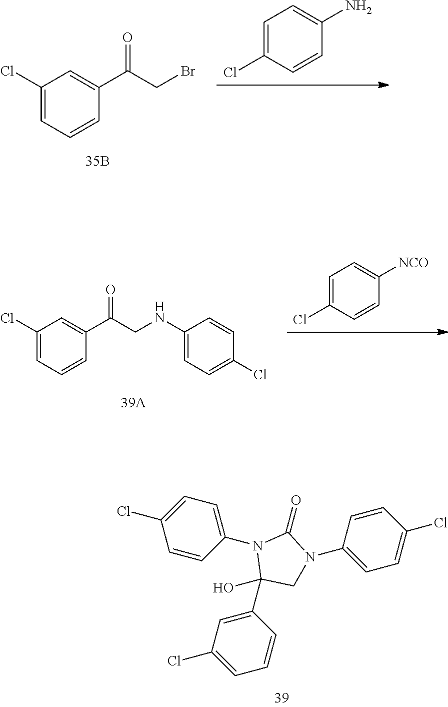

Compounds 39A and 39 were synthesized by employing the procedures described for Compounds 1B and 1 using Compound 35B, 4-chloroanilline, 39A, and 1-chloro-4-isocyanatobenzene in lieu of Compound 1A, 4-brommoanilline, 1B, and 1-bromo-4-isocyanatobenzene. Compound 39A: LC-MS (ESI) m/z: 280 [M+H]⁺. Compound 39: LC-MS (ESI) m/z: 433 [M+H]⁺; ¹H-NMR (DMSO-d₆, 400 MHz): δ (ppm): 4.04 (d, J=10.4 Hz, 1H), 4.17 (d, J=10.4 Hz, 1H), 7.28-7.44 (m, 8H), 7.48-7.53 (m, 1H), 7.65-7.71 (m, 3H), 7.83 (s, 1H).

### Example 40

### Synthesis of 1,3-bis(4-chlorophenyl)-4-ethyl-5-(3-(trifluoromethyl)phenyl)-1,3-dihydro-2H-imidazol-2-one

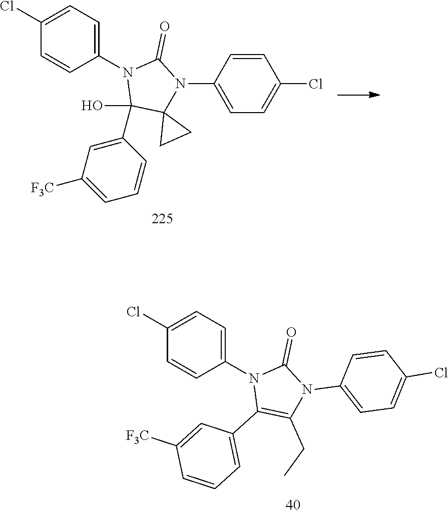 [SMILES: O=C1N(c2ccc(Cl)cc2)C2(CC2)C(O)(c2cccc(C(F)(F)F)c2)N1c1ccc(Cl)cc1 ; CCc1c(-c2cccc(C(F)(F)F)c2)n(-c2ccc(Cl)cc2)c(=O)n1-c1ccc(Cl)cc1]

To a stirred solution of Compound 225 (50 mg, 0.1 mmol) in dichloromethane (5 mL) was added diethylsilane (116 mg, 1 mmol) and boron trifluoride ether solution (28 mg, 0.2 mmol). The mixture was stirred at room temperature overnight. The mixture was diluted with dichloromethane (100 mL), washed with saturated NaHCO₃ solution (50 mL), dried over anhydrous Na₂SO₄, filtered, and concentrated under reduced pressure. The crude product was purified with preparative HPLC to give Compound 40. LC-MS (ESI) m/z: 477 [M+H]⁺. ¹H-NMR (400 MHz, CDCl₃): δ (ppm) 0.87 (t, J=7.2 Hz, 3H), 2.50 (q, J=7.2 Hz, 2H), 7.09-7.11 (m, 2H), 7.21-7.28 (m, 3H), 7.37-7.42 (m, 4H), 7.37-7.42 (m, 3H).

### Example 41

### Synthesis of 1,3-bis(4-chlorophenyl)-4-(methoxymethyl)-5-(3-(trifluoromethyl)phenyl)-1,3-dihydro-2H-imidazol-2-one

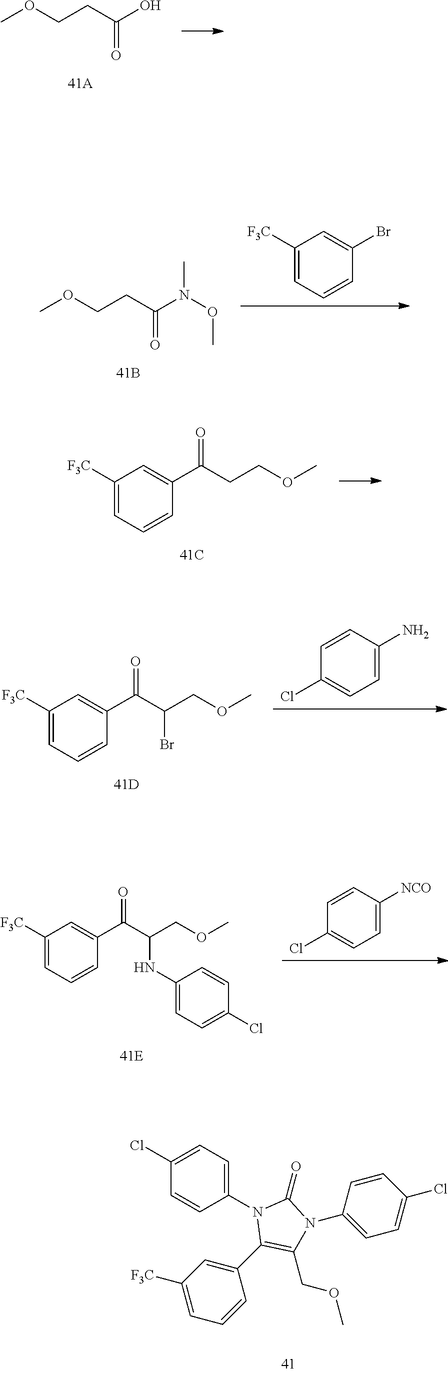

To a solution of 3-methoxypropanoic acid 41A (5.00 g, 48.08 mmol) in dichloromethane (50 mL) and DMF (0.5 mL) was dropped oxalyl dichloride (9.09 g, 72.12 mmol) at 0° C. The mixture was stirred at 25° C. for 16 hours and concentrated to give a crude product. The crude 3-methoxypropenoyl chloride (5.51 g, 45.20 mmol) was added dropwise to a suspension of N,O-dimethylhydroxylamine hydrochloride (6.58 g, 67.80 mmol) and Et₃N (9.94 g, 90.40 mmol) in dichloromethane (100 mL) at room temperature. The mixture was stirred at 25° C. for 3 hours and filtered. The filtrate was concentrated and the residue was purified with flash column chromatography in silica gel (ethyl acetate in petroleum ether, from 0% to 30% v/v) to give Compound 41B. LC-MS (ESI) m/z: non-ionizable compound under routine conditions used; ¹H-NMR (CDCl₃, 400 MHz): δ (ppm) 2.72 (d, J=6.4 Hz, 2H), 3.19 (s, 3H), 3.37 (s, 3H), 3.68-3.72 (m, 5H).

Compounds 41C, 41D, 41E, and 41 were synthesized by employing the procedures described for Compounds 59B, 13B, 1B, and 1 using 1-bromo-3-(trifluoromethyl)benzene, Compounds 41B, 41C, 41D, 4-chloroaniline using NMP as solvent at 55° C., 1-chloro-4-isocyanatobenzene, and Compound 41E in lieu of Compound 59A, N-methoxy-N-methylacetamide, Compounds 13A, 1A, 4-bromoaniline using EtOH as solvent at 25° C., 1-bromo-4-isocyanatobenzene, and Compound 1B. Compound 41C. LC-MS (ESI) m/z: 233 [M+H]⁺; ¹H-NMR (CDCl₃, 400 MHz): δ (ppm) 3.26 (t, J=6.4 Hz, 2H), 3.64 (s, 3H), 3.83 (t, J=6.4 Hz, 2H), 7.62 (t, J=8.0 Hz, 1H), 7.82 (t, J=8.0 Hz, 1H), 8.15 (d, J=8.0 Hz, 1H), 8.23 (s, 1H). Compound 41D. LC-MS (ESI) m/z: 311 [M+H]⁺; ¹H-NMR (CDCl₃, 400 MHz): δ (ppm) 3.44 (s, 3H), 3.86-3.90 (m, 1H), 4.11-4.16 (m, 1H), 5.16-5.20 (m, 1H), 7.65 (t, J=8.0 Hz, 1H), 7.86 (d, J=8.0 Hz, 1H), 8.19 (d, J=7.6 Hz, 1H), 8.27 (s, 1H). Compound 41E. LC-MS (ESI) m/z: 358 [M+H]⁺; ¹H-NMR (CDCl₃, 400 MHz): δ (ppm) 3.28 (s, 3H), 3.72-3.78 (m, 2H), 4.81 (d, J=8.0 Hz, 1H), 5.06-5.11 (m, 1H), 6.62 (d, J=8.8 Hz, 2H), 7.13 (d, J=8.8 Hz, 2H), 77.65 (t, J=8.0 Hz, 1H), 77.87 (d, J=7.6 Hz, 1H), 8.19 (d, J=8.0 Hz, 1H), 8.27 (s, 1H). Compound 41. LC-MS (ESI) m/z: 493 [M+H]⁺; ¹H-NMR (CDCl₃, 400 MHz): δ (ppm) 3.32 (s, 3H), 3.98 (s, 2H), 7.15 (d, J=9.2 Hz, 2H), 7.22-7.27 (m, 1H), 7.29-7.32 (m, 2H), 7.47 (t, J=8.8 Hz, 1H), 7.50-7.55 (m, 2H), 7.57-7.59 (m, 3H), 7.63 (s, 1H).

### Example 42

### Synthesis of 1,3-bis(4-bromophenyl)-4-(3,5-dimethylphenyl)-4-hydroxy-5-methylimidazolidin-2-one, (5S)-1,3-bis(4-bromophenyl)-4-(3,5-dimethylphenyl)-4-hydroxy-5-methylimidazolidin-2-one, and (5R)-1,3-bis(4-bromophenyl)-4-(3,5-dimethylphenyl)-4-hydroxy-5-methylimidazolidin-2-one

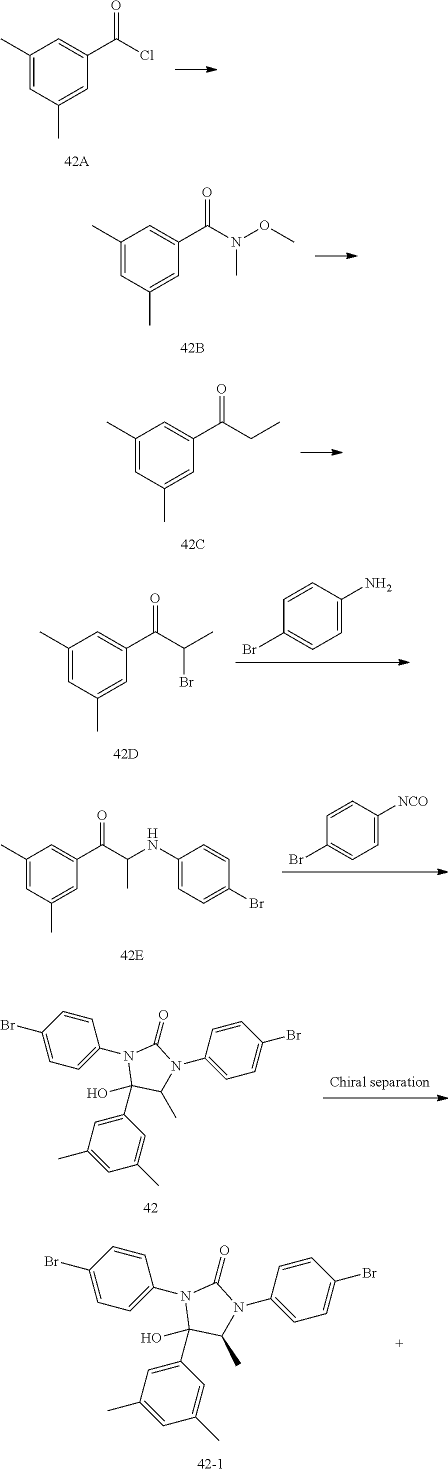

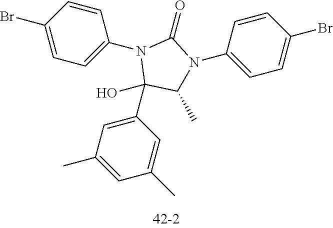 [SMILES: Cc1cc(C)cc(C2(O)[C@@H](C)N(c3ccc(Br)cc3)C(=O)N2c2ccc(Br)cc2)c1]

To a mixture of N,O-dimethylhydroxylamine hydrochloride (1.38 g, 14.28 mmol) and triethylamine (4 mL) in dichloromethane (30 mL) was dripped 3,5-dimethylbenzoyl chloride 42A (2.00 g, 11.90 mmol) at 0° C. The mixture was stirred at 25° C. for 2 hours, diluted with ethyl acetate (160 mL), and filtered. The filtrate was concentrated under reduced pressure. The residue was purified with flash column chromatography on silica gel (ethyl acetate in petroleum ether, from 0% to 20% v/v) to give Compound 42B: LC-MS (ESI) m/z: 194 [M+H]⁺; ¹H-NMR (CDCl₃, 400 MHz): δ (ppm) 2.34 (s, 6H), 3.34 (s, 3H), 3.58 (s, 3H), 7.08 (s, 1H), 7.24-7.26 (m, 2H).

To a solution of Compound 42B (1.00 g, 5.18 mmol) in THF (20 mL) was added dropwise EtMgBr solution (1 M in ether 21 mL, 20.72 mmol) at −40° C. under nitrogen. The mixture was stirred at −10° C. for 2 hours, quenched with saturated NH₄Cl solution (20 mL), and diluted with ethyl acetate (160 mL). The organic layer was washed with water (200 mL) and brine (200 mL), dried over anhydrous sulfate sodium, filtered, and concentrated under reduced pressure. The residue was purified with flash column chromatography on silica gel (ethyl acetate in petroleum ether, from 0% to 15% v/v) to afford Compound 42C: LC-MS (ESI) m/z: 163 [M+H]⁺; ¹H-NMR (CDCl₃, 400 MHz): δ (ppm) 1.21 (t, J=7.2 Hz, 3H), 2.37 (s, 6H), 2.95-3.00 (m, 2H), 7.19 (s, 1H), 7.57 (s, 2H).

Compounds 42D, 42E, and 42 were synthesized by employing the procedures described for Compounds 13B, 13C, and 1 using Compounds 42C, 42D, and 42E in lieu of Compounds 13A, 13B, and 1B. Compound 42D: LC-MS (ESI) m/z: 241 [M+H]⁺; ¹H-NMR (CDCl₃, 400 MHz): δ (ppm) 1.21 (d, J=7.2 Hz, 3H), 2.38 (s, 6H), 5.26-5.32 (m, 1H), 7.23 (s, 1H), 7.62 (s, 2H). Compound 42E: LC-MS (ESI) m/z: 332 [M+H]⁺; ¹H-NMR (CDCl₃, 400 MHz): δ (ppm) 1.45 (d, J=6.8 Hz, 3H), 2.40 (s, 6H), 4.75-4.77 (m, 1H), 5.00-5.08 (m, 1H), 6.55 (d, J=8.8 Hz, 3H), 7.23-7.26 (m, 3H), 7.59 (s, 1H). Compound 42: LC-MS (ESI) m/z: 529 [M+H]⁺; ¹H-NMR (DMSO-d₆, 400 MHz): δ (ppm) 1.06 (d, J=6.8 Hz, 3H), 2.21 (s, 6H), 4.29-4.34 (m, 1H), 6.88 (s, 1H), 7.18-7.21 (m, 3H), 7.36-7.43 (m, 6H), 7.56 (d, J=8.8 Hz, 2H).

Compound 42 was separated with chiral HPLC to give Compound 42-1 and Compound 42-2. Compound 42-1: LC-MS (ESI) m/z: 529 [M+H]⁺; ¹H-NMR (DMSO-d₆, 400 MHz): δ (ppm) 1.06 (d, J=6.8 Hz, 3H), 2.21 (s, 6H), 4.29-4.34 (m, 1H), 6.88 (s, 1H), 7.18-7.21 (m, 3H), 7.36-7.39 (m, 6H), 7.56 (d, J=8.8 Hz, 2H); Chiral separation condition: MeOH contained 0.2% Methanol ammonia; IC (250*4.6 mm, 5 μm); retention time: 1.81 minutes. Compound 42-2: LC-MS (ESI) m/z: 529 [M+H]⁺; ¹H-NMR (DMSO-d₆, 400 MHz): δ (ppm) 1.06 (d, J=6.8 Hz, 3H), 2.21 (s, 6H), 4.28-4.34 (m, 1H), 6.88 (s, 1H), 7.18-7.21 (m, 3H), 7.36-7.39 (m, 6H), 7.56 (d, J=8.8 Hz, 2H); Chiral separation condition: MeOH contained 0.2% Methanol ammonia; IC (250*4.6 mm, 5 μm); retention time: 4.90 minutes.

### Example 43

### Synthesis of 1-(4-bromophenyl)-3-(4-chlorophenyl)-4-hydroxy-4-phenylimidazolidin-2-one

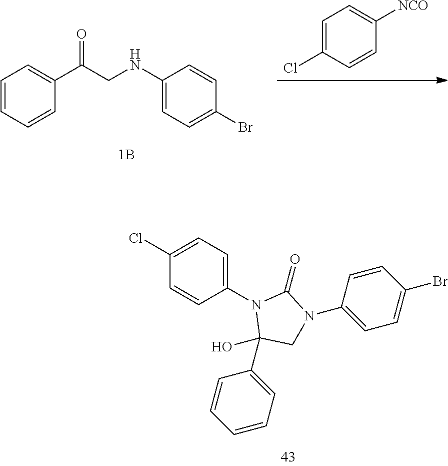

Compound 43 was synthesized by employing the procedure described for Compound 1 using 1-chloro-4-isocyanatobenzene in lieu of 1-bromo-4-isocyanatobenzene. LC-MS (ESI) m/z: 443 [M+H]⁺; ¹H-NMR (DMSO-d₆, 400 MHz): δ (ppm) 4.03-4.14 (m, 2H), 7.25-7.39 (m, 7H), 7.53-7.58 (m, 4H), 7.65-7.67 (m, 3H).

### Example 44

### Synthesis of 3-(1,3-bis(4-chlorophenyl)-5-isopropyl-2-oxo-2,3-dihydro-1H-imidazol-4-yl)benzonitrile

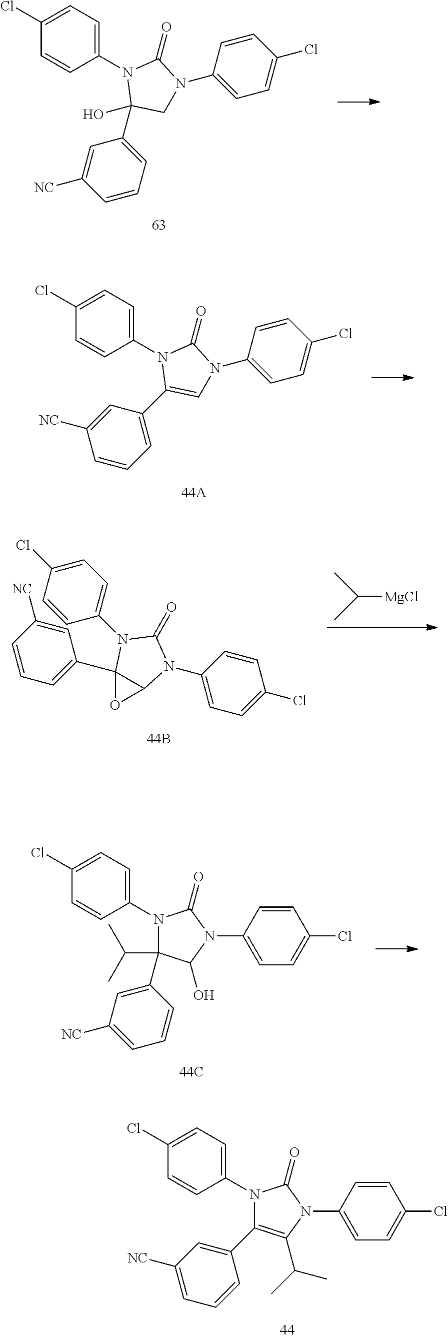

Compounds 44A, 44B, and 44C were synthesized by employing the procedures described for Compounds 255, 86B, and 86 using Compounds 63, 44A, 44B, and isopropylmagnesium chloride in lieu of Compounds 1, 86A, and 86B, and phenylmagnesium bromide. Compound 44A. LC-MS (ESI) m/z: 406 [M+H]⁺; ¹H-NMR (DMSO-d₆, 400 MHz): δ (ppm) 7.27-7.33 (m, 3H), 7.47-7.60 (m, 5H), 7.72-7.76 (m, 2H), 7.82 (s, 1H), 7.87-7.89 (m, 2H).

Compound 44B. LC-MS (ESI) m/z: 422 [M+H]⁺. Compound 44C. LC-MS (ESI) m/z: 466[M+H]⁺; ¹H-NMR (acetone-d₆, 400 MHz): δ (ppm) 1.06 (d, J=7.2 Hz, 3H), 1.19 (d, J=6.8 Hz, 3H), 2.19-2.26 (m, 1H), 5.14 (s, 1H), 5.74 (s, 1H), 7.24 (d, J=9.2 Hz, 2H), 7.43 (d, J=8.8 Hz, 2H), 7.56-7.69 (m, 5H), 7.71-7.82 (m, 1H), 7.84-7.88 (m, 2H).

To a solution of Compound 44C (33 mg, 0.07 mmol) in DCM (10 mL) was added TFA (2 mL) at 0° C. The mixture was stirred at room temperature for 2 hours and evaporated under reduced pressure. The residue was purified with preparative HPLC to yield Compound 44. LC-MS (ESI) m/z: 448 [M+H]⁺; ¹H-NMR (Acetone-d₆, 400 MHz): δ (ppm) 1.04 (d, J=7.2 Hz, 6H), 2.88-2.94 (m, 1H), 7.21 (d, J=8.8 Hz, 2H), 7.32 (d, J=8.8 Hz, 2H), 7.52-7.64 (m, 6H), 7.74-7.75 (m, 2H).

### Example 45

### Synthesis of 1,3-bis(4-bromophenyl)-4-ethyl-4-hydroxy-5-phenylimidazolidin-2-one, (5S)-1,3-bis(4-bromophenyl)-4-ethyl-4-hydroxy-5-phenylimidazolidin-2-one, and (5R)-1,3-bis(4-bromophenyl)-4-ethyl-4-hydroxy-5-phenylimidazolidin-2-one

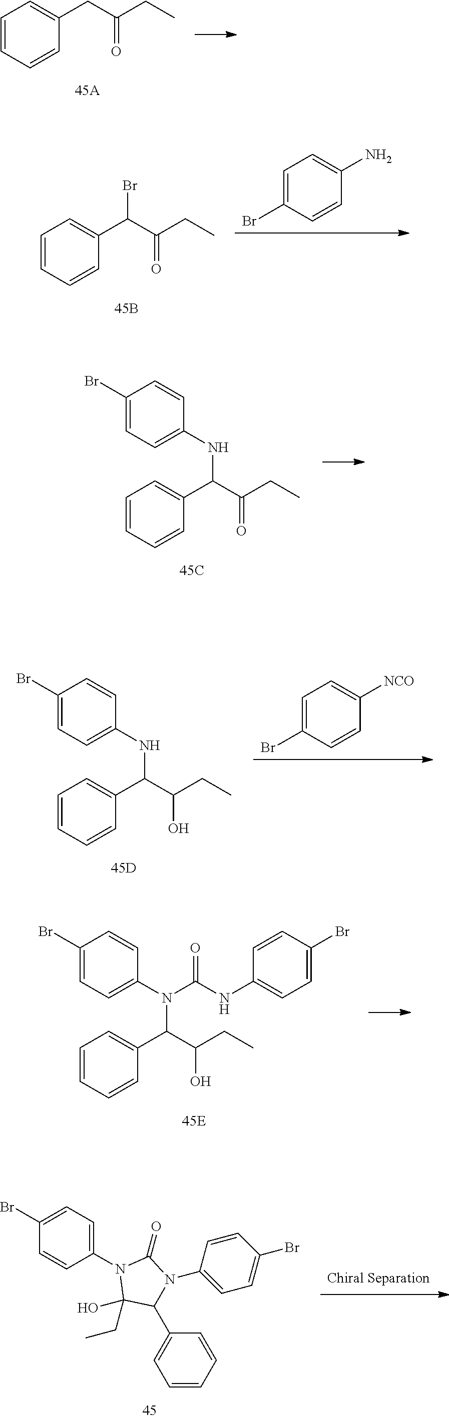

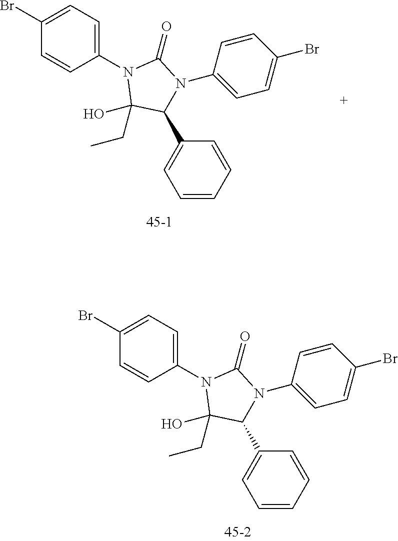 [SMILES: CCC1(O)[C@H](c2ccccc2)N(c2ccc(Br)cc2)C(=O)N1c1ccc(Br)cc1 ; CCC1(O)[C@@H](c2ccccc2)N(c2ccc(Br)cc2)C(=O)N1c1ccc(Br)cc1]

Compounds 45B, 45C, 45D, 45E, and 45 were synthesized by employing the procedures described for Compounds 13B, 1B, 14C, 1, and 14 using Compounds 45A, 45B, 45C, 45D, and 45E in lieu of Compounds 13A, 1A, 14B, 1B, and 14D. Compound 45B: LC-MS (ESI) m/z: 227 [M+H]; ¹H-NMR (CDCl₃, 400 MHz): δ (ppm) 1.06 (t, J=7.2 Hz, 3H), 2.60-2.66 (m, 2H), 5.45 (s, 1H), 7.30-7.38 (m, 5H). Compound 45C: LC-MS (ESI) m/z: 318 [M+H]⁺; ¹H-NMR (CDCl₃, 400 MHz): δ (ppm) 0.98 (t, J=7.6 Hz, 3H), 2.43-2.47 (m, 2H), 4.95 (d, J=4.4 Hz, 1H), 5.32-5.33 (m, 1H), 6.39-6.42 (m, 2H), 7.13-7.15 (m, 2H), 7.30-7.42 (m, 5H). Compound 45D: LC-MS (ESI) m/z: 320 [M+H]⁺. Compound 45E: LC-MS (ESI) m/z: 517 [M+H]⁺; ¹H-NMR (CDCl₃, 400 MHz): δ (ppm) 1.10 (t, J=7.2 Hz, 3H), 1.61-1.69 (m, 1H), 1.88-1.94 (m, 1H), 2.46 (d, J=3.6 Hz, 1H), 4.20-4.24 (m, 1H), 5.29 (d, J=7.2 Hz, 1H), 5.83 (s, 1H), 6.76 (d, J=8.4 Hz, 2H), 7.13-7.15 (m, 2H), 7.21-7.23 (m, 2H), 7.27-7.29 (m, 3H), 7.33-7.35 (m, 2H), 7.49 (d, J=8.4 Hz, 2H). Compound 45: LC-MS: (ESI) m/z: 515 [M+H]⁺.

Compound 45 was separated by chiral HPLC to give Compound 45-1 and Compound 45-2. Compound 45-1: LC-MS (ESI) m/z: 515 [M+H]⁺; ¹H-NMR (acetone-d₆, 500 MHz): δ (ppm) 1.05 (t, J=8.0 Hz, 3H), 1.92-2.01 (m, 2H), 4.94 (s, 1H), 5.57 (s, 1H), 7.34-7.38 (m, 5H), 7.41-7.45 (m, 4H), 7.58-7.61 (m, 4H). Chiral separation condition: co-solvent: MeOH contained 0.2% Methanol Ammonia; IC (4.6×50 mm, 5 μm); retention time: 2.89 minutes. Compound 45-2: LC-MS (ESI) m/z: 515 [M+H]⁺; ¹H-NMR acetone-d₆, 500 MHz): δ (ppm) 1.05 (t, J=8.0 Hz, 3H), 1.94-2.00 (m, 2H), 4.94 (s, 1H), 5.57 (s, 1H), 7.29-7.39 (m, 5H), 7.41-7.46 (m, 4H), 7.58-7.62 (m, 4H). Chiral separation condition: co-solvent: MeOH contained 0.2% Methanol Ammonia; IC (4.6×50 mm, 5 μm); retention time: 3.96 minutes.

### Example 46

### Synthesis of 3-(1,3-bis(4-bromophenyl)-5-isopropyl-2-oxo-2,3-dihydro-1H-imidazol-4-yl)benzonitrile

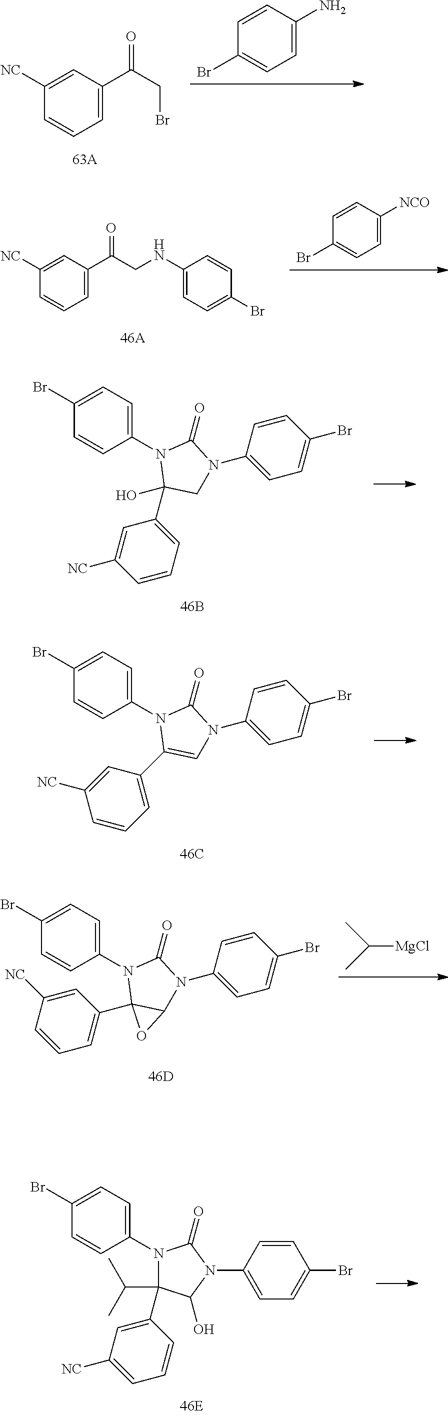

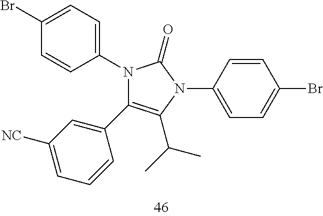 [SMILES: CC(C)c1c(-c2cccc(C#N)c2)n(-c2ccc(Br)cc2)c(=O)n1-c1ccc(Br)cc1]

Compounds 46A, 46B, 46C, 46D, and 46E were synthesized by employing the procedures described for Compounds 1B, 12, 255, 86B, and 86 using Compounds 63A using NMP as solvent, 46A using NaHCO₃ as base, 1-bromo-4-isocyanatobenzene, Compounds 46B, 46C, 46D, and isopropylmagnesium chloride in lieu of Compound 1A using EtOH as solvent, 12B using DIPEA as base, 1-chloro-4-isocyanatobenzene, Compounds 1, 86A, 86B, and phenylmagnesium bromide. Compound 46A. LC-MS (ESI) m/z: 315 [M+H]⁺. Compound 46B. LC-MS (ESI) m/z: 512 [M+H]⁺. Compound 46C. LC-MS (ESI) m/z: 494 [M+H]⁺; ¹H-NMR (CDCl₃, 400 MHz): δ (ppm) 6.87 (s, 1H), 7.10-7.12 (m, 3H), 7.38-7.59 (m, 9H). Compound 46D. LC-MS (ESI) m/z: 510 [M+H]⁺; ¹H-NMR (CDCl₃, 400 MHz): δ (ppm) 5.65 (s, 1H), 7.33-7.69 (m, 12H). Compound 46E. LC-MS (ESI) m/z: 554 [M+H]⁺; ¹H-NMR (acetone-d₆, 400 MHz): δ (ppm) 1.05 (d, J=7.2 Hz, 3H), 1.19 (d, J=6.4 Hz, 3H), 2.19-2.26 (m, 1H), 5.15 (s, 1H), 5.73 (s, 1H), 7.37-7.40 (m, 2H), 7.54-7.60 (m, 7H), 7.69-7.71 (m, 1H), 7.81-7.88 (m, 2H). Compound 46. LC-MS (ESI) m/z: 536 [M+H]⁺; ¹H-NMR (acetone-d₆, 400 MHz): δ (ppm) 1.04 (d, J=6.8 Hz, 6H), 2.88-2.92 (m, 1H), 7.14-7.17 (m, 2H), 7.45-7.49 (m, 4H), 7.55-7.64 (m, 2H), 7.74-7.77 (m, 4H).

### Example 47

### Synthesis of 1,3-bis(4-bromophenyl)-4-(3-ethylphenyl)-4-hydroxyimidazolidin-2-one

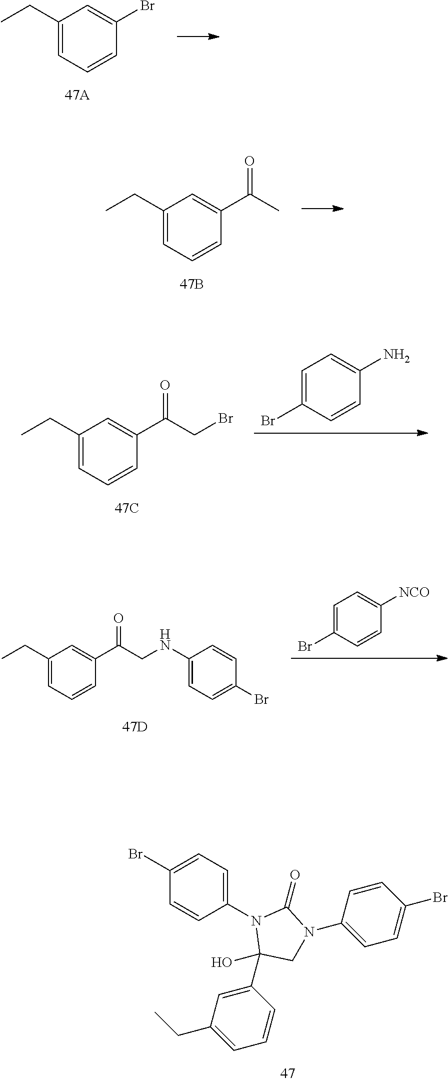

A mixture of tributyl(1-ethoxyvinyl)stannane (4.3 g, 12 mmol), 1-bromo-3-ethylbenzene 47A (2.0 g, 10.8 mmol), and dichlorobis(triphenylphosphine)palladium (100 mg, 0.12 mmol) in toluene (20 mL) was stirred at 100° C. under nitrogen for 18 hours. After cooled down to room temperature, the reaction mixture was quenched with hydrochloric acid (1 N) and extracted with ethyl acetate (50 mL×2). The combined organic layers was dried over sodium sulfate, filtered, and concentrated under reduced pressure. The residue was purified with flash column chromatography on silica gel (ethyl acetate in petroleum ether, 10% v/v) to furnish Compound 47B: LC-MS (ESI) m/z: 149 [M+H]⁺; ¹H-NMR (CDCl₃, 400 MHz): δ (ppm) 1.27 (t, J=7.6 Hz, 3H), 2.61 (s, 3H), 2.71 (q, J=7.6 Hz, 2H), 7.38-7.40 (m, 2H), 7.76-7.80 (m, 2H).

Compounds 47C, 47D, and 47 were synthesized by employing the procedures described for Compounds 13B, 1B, and 1 using Compounds 47B, 47C, and 47D in lieu of Compounds 13A, 1A, and 1B. Compound 47C: LC-MS (ESI) m/z: 227 [M+H]⁺; ¹H-NMR (CDCl₃, 400 MHz): δ (ppm) 1.27 (t, J=7.6 Hz, 3H), 2.72 (q, J=7.2 Hz, 2H), 4.46 (s, 2H), 7.38-7.49 (m, 2H), 7.78-7.90 (m, 2H). Compound 47D: LC-MS (ESI) m/z: 318 [M+H]⁺; ¹H-NMR (DMSO-d₆, 400 MHz): δ (ppm) 1.22 (t, J=7.6 Hz, 3H), 2.70 (q, J=7.6 Hz, 2H), 4.66 (d, J=5.6 Hz, 2H), 6.10 (t, J=5.2 Hz, 1H), 6.66 (d, J=9.2 Hz, 2H), 7.20 (d, J=8.8 Hz, 2H), 7.47-7.54 (m, 2H), 7.87-7.89 (m, 2H). Compound 47: LC-MS (ESI) m/z: 515 [M+H]⁺; ¹H-NMR (DMSO-d₆, 400 MHz): δ (ppm) 1.09 (t, J=7.6 Hz, 3H), 2.52 (q, J=7.2 Hz, 2H), 4.03 (d, J=10.8 Hz, 1H), 4.12 (d, J=10.4 Hz, 1H), 7.08 (d, J=7.6 Hz, 1H), 7.21 (t, J=7.6 Hz, 1H), 7.31-7.42 (m, 6H), 7.53 (d, J=8.8 Hz, 2H), 7.64-7.67 (m, 3H).

### Example 48

### Synthesis of 3-(1,3-bis(4-chlorophenyl)-5-cyclopropyl-2-oxo-2,3-dihydro-1H-imidazol-4-yl)benzonitrile

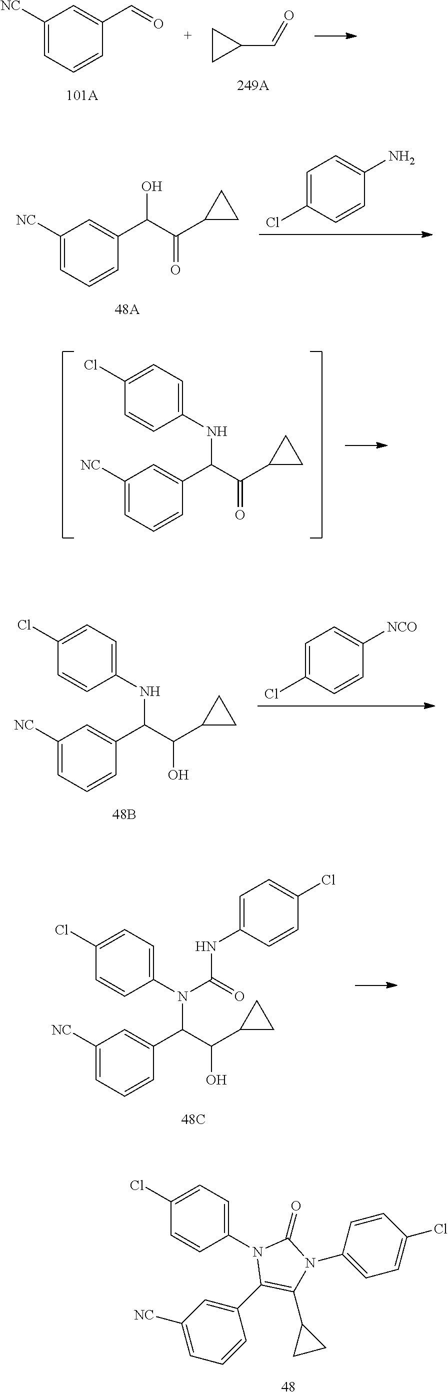

Compound 48A was synthesized by employing the procedure described for Compound 249C using Compound 101A in lieu of Compound 249B. LC-MS (ESI) m/z: 202 [M+H]⁺; ¹H-NMR (CDCl₃, 400 MHz): δ (ppm) 0.85-0.90 (m, 1H), 1.02-1.09 (m, 2H), 1.19-1.24 (m, 1H), 1.81-1.87 (m, 1H), 4.45 (brs., 1H), 5.30 (s, 1H), 7.51-7.53 (m, 1H), 7.54-7.61 (m, 2H), 7.67-7.69 (m, 1H).

A solution of Compound 48A (2.5 g, 12.4 mmol) and 4-chloroaniline (2.14 g, 13.7 mmol) in ethanol (10 mL) in a sealed tube was stirred at 130° C. for 16 hours. The mixture was cooled down to room temperature and sodium borohydride (1.38 g, 37.2 mmol) was added. The reaction mixture was stirred at 5° C. for 1 hour, quenched with aqueous HCl solution (1 N, 50 mL), and extracted with ethyl acetate (50 mL×3). The combined extracts was washed with water (30 mL×2) and brine (30 mL), dried over anhydrous sodium sulfate, filtered, and concentrated. The residue was purified with flash column chromatography on silica gel (ethyl acetate in petroleum ether, 20% v/v) to give Compound 48B. LC-MS (ESI) m/z: 313 [M+H]⁺; ¹H-NMR (CDCl₃, 400 MHz): δ (ppm) 0.06-0.63 (m, 5H), 3.02-3.27 (m, 1H), 4.46-4.48 (m, 1H), 6.39-6.45 (m, 2H), 6.39-6.45 (m, 2H), 7.01-7.06 (m, 2H), 7.42 (t, J=8.0 Hz, 1H), 7.55-7.73 (m, 3H).

Compounds 48C and 48 were synthesized by employing the procedures described for Compounds 1 and 14 using Compound 48B, 1-chloro-4-isocyanatobenzene, and Compound 48C in lieu of Compound 1B, 1-bromo-4-isocyanatobenzene, and Compound 14D. Compound 48C. LC-MS (ESI) m/z: 466 [M+H]⁺. Compound 48. LC-MS (ESI) m/z: 446 [M+H]⁺; ¹H-NMR (CD₃OD, 400 MHz): δ (ppm) 0.01-0.05 (m, 2H), 0.58-0.63 (m, 2H), 1.83-1.90 (m, 1H), 7.19-7.23 (m, 2H), 7.37-7.49 (m, 4H), 7.56 (s, 4H), 7.6-7.68 (m, 2H).

### Example 49

### Synthesis of 1,3-bis(4-bromophenyl)-4-hydroxy-5-methyl-4-(thiophen-3-yl)imidazolidin-2-one, (5S)-1,3-bis(4-bromophenyl)-4-hydroxy-5-methyl-4-(thiophen-2-yl)imidazolidin-2-one, and (5R)-1,3-bis(4-bromophenyl)-4-hydroxy-5-methyl-4-(thiophen-2-yl)imidazolidin-2-one

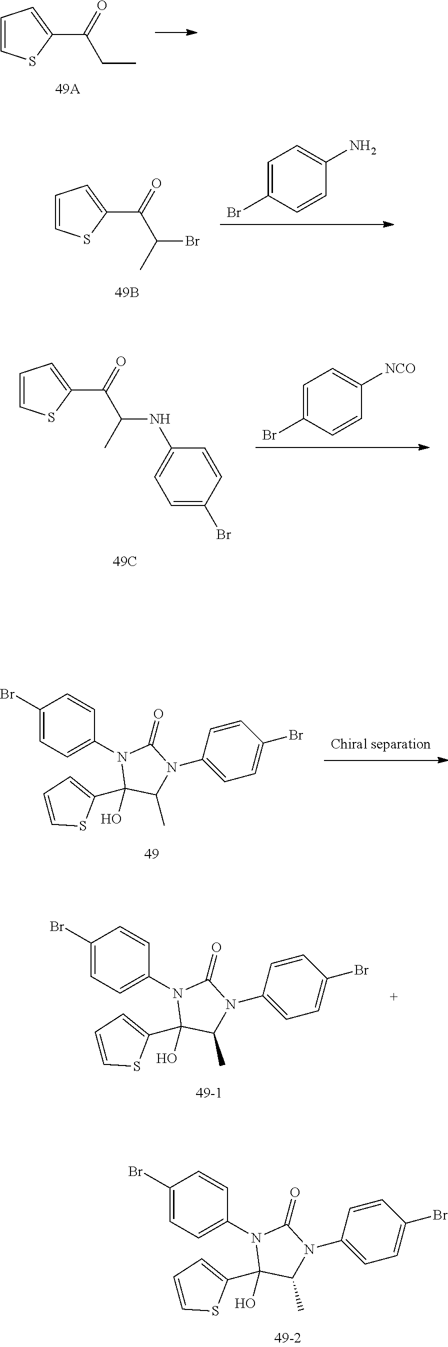

Compounds 49B, 49C, and 49 were synthesized by employing the procedures described for Compounds 13B, 13C, and 1 using Compounds 49A, 49B, and 49C in lieu of Compounds 13A, 13B, and 1B. Compound 49B: LC-MS (ESI) m/z: 219 [M+H]⁺; ¹H-NMR (CDCl₃, 400 MHz): δ (ppm) 1.21 (d, J=6.8 Hz, 3H), 5.12-5.17 (m, 1H), 7.15-7.17 (m, 1H), 7.70 (d, J=4.8 Hz, 1H), 7.85 (d, J=4.8 Hz, 1H). Compound 49C: LC-MS (ESI) m/z: 310 [M+H]⁺; ¹H-NMR (CDCl₃, 400 MHz): δ (ppm) 1.54 (d, J=6.8 Hz, 3H), 4.55 (d, J=7.6 Hz, 1H), 4.78-4.85 (m, 1H), 6.52 (d, J=8.4 Hz, 2H), 7.17-7.26 (m, 3H), 7.71 (d, J=4.8 Hz, 1H), 7.87 (d, J=4.8 Hz, 1H). Compound 49. LC-MS (ESI) m/z: 507 [M+H]⁺; ¹H-NMR (DMSO-d₆, 400 MHz): δ (ppm) 1.15 (d, J=6.4 Hz, 3H), 4.51-4.57 (m, 1H), 6.87-6.97 (m, 2H), 7.37-7.46 (m, 7H), 7.57-7.89 (m, 3H).

Compound 49 was separated by chiral HPLC to give Compound 49-1 and Compound 49-2. Compound 49-1: LC-MS (ESI) m/z: 507 [M+H]⁺; ¹H-NMR (Acetone-d₆, 400 MHz): δ (ppm) 0.94-1.34 (dd, J=6.4 Hz, 3H), 4.59-4.65 (m, 1H), 6.49-6.99 (m, 2H), 7.23-7.46 (m, 7H), 7.56-7.88 (m, 3H); Chiral separation condition: MeOH contained 0.2% Methanol ammonia; OZ-H (250*4.6 mm, 5 μm); retention time: 2.17 minutes (80%), 2.70 minutes (20%). Compound 49-2: LC-MS (ESI) m/z: 507 [M+H]⁺; ¹H-NMR (Acetone-d₆, 400 MHz): δ (ppm) 0.95-1.35 (dd, J=6.4 Hz, 3H), 4.60-4.65 (m, 1H), 6.49-6.99 (m, 2H), 7.23-7.46 (m, 7H), 7.53-7.88 (m, 3H); Chiral separation condition: MeOH contained 0.2% Methanol ammonia; OZ-H (250*4.6 mm, 5 μm); retention time: 2.86 minutes (20%), 3.44 minutes (80%).

### Example 50

### Synthesis of 1,3-bis(4-chlorophenyl)-4-isopropyl-5-(3-(trifluoromethoxy)phenyl)-1,3-dihydro-2H-imidazol-2-one

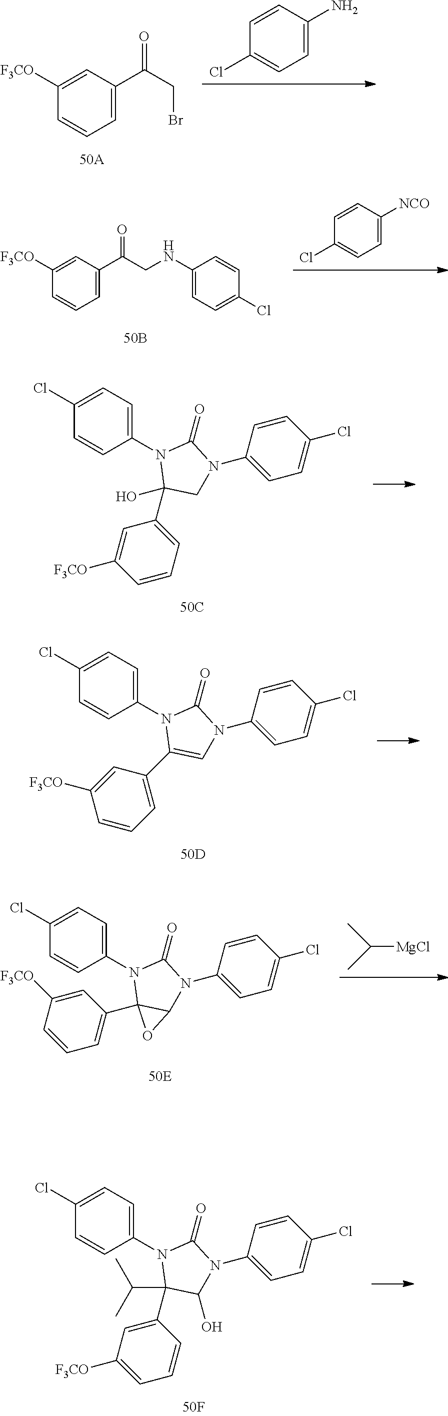

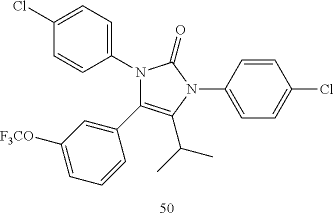 [SMILES: CC(C)c1c(-c2cccc(OC(F)(F)F)c2)n(-c2ccc(Cl)cc2)c(=O)n1-c1ccc(Cl)cc1]

Compounds 50B, 50C, 50D, 50E, and 50F were synthesized by employing the procedures described for Compounds 1B, 12, 255, 86B, and 86 using Compounds 50A, 4-chloroaniline, Compound 50B using NaHCO₃ as base, Compounds 50C, 50D, 50E, and isopropylmagnesium chloride in lieu of Compound 1A, 4-bromoaniline, Compound 12B using DIPEA as base, Compounds 1, 86A, 86B, and phenylmagnesium bromide. Compound 50B. LC-MS (ESI) m/z: 330 [M+H]⁺. Compound 50C. LC-MS: (ESI) m/z: 483 [M+H]⁺. Compound 50D. LC-MS (ESI) m/z: 465 [M+H]⁺; ¹H-NMR (Acetone-d₆, 400 MHz): δ (ppm) 7.14 (s, 1H), 7.24-7.28 (m, 2H), 7.28-7.34 (m, 2H), 7.45-7.56 (m, 6H), 7.94 (d, J=9.2 Hz, 2H). Compound 50E. LC-MS (ESI) m/z: 481 [M+H]⁺; ¹H-NMR (CDCl₃, 400 MHz): δ (ppm) 5.61 (s, 1H), 7.24-7.33 (m, 6H), 7.39-7.47 (m, 6H). Compound 50F. LC-MS (ESI) m/z: 525 [M+H]⁺; ¹H-NMR (Acetone-d₆, 400 MHz): δ (ppm) 1.05 (d, J=6.8 Hz, 3H), 1.18 (d, J=6.8 Hz, 3H), 2.19-2.25 (m, 1H), 5.71 (s, 1H), 7.23-7.25 (m, 3H), 7.42-7.50 (m, 5H), 7.60-7.64 (m, 4H). Compound 50. LC-MS (ESI) m/z: 507 [M+H]⁺; ¹H-NMR (Acetone-d₆, 400 MHz): δ (ppm) 1.04 (d, J=7.2 Hz, 6H), 2.86-2.93 (m, 1H), 1.90-7.23 (m, 3H), 7.29-7.37 (m, 4H), 7.49-7.54 (m, 3H), 7.60-7.62 (2H).

### Example 51

### Synthesis of 1,3-bis(4-bromophenyl)-4-hydroxy-4-phenyl-5-propylimidazolidin-2-one, (5S)-1,3-bis(4-bromophenyl)-4-hydroxy-4-phenyl-5-propylimidazolidin-2-one, and (5R)-1,3-bis(4-bromophenyl)-4-hydroxy-4-phenyl-5-propylimidazolidin-2-one

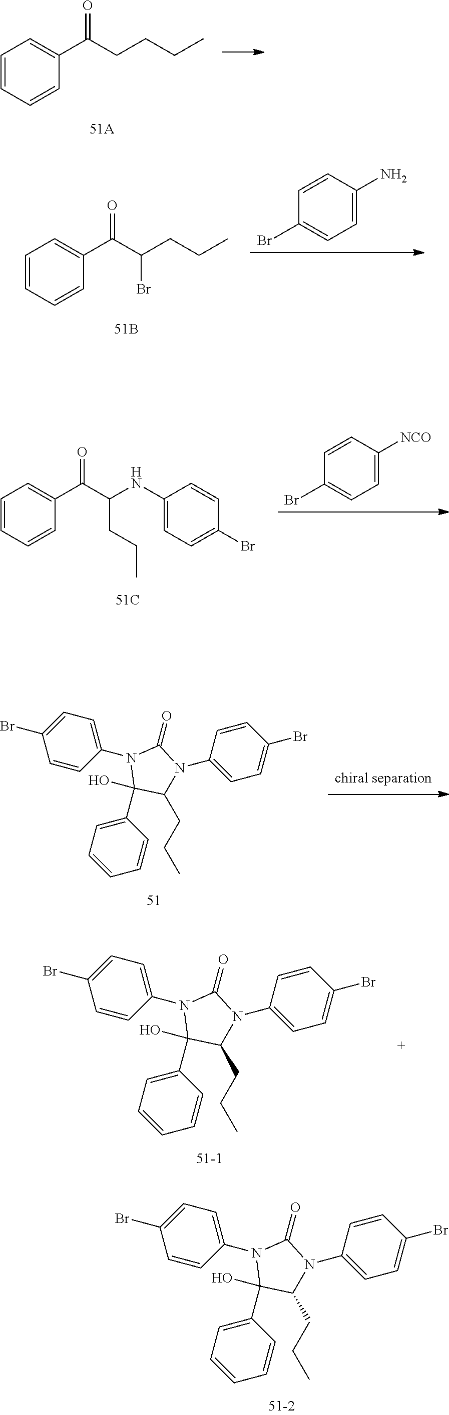

Compounds 51B, 51B, and 51 were synthesized by employing the procedures described for Compounds 13B, 1B, and 1 using Compounds 51A, 51B, heated at 60° C., and 51C in lieu of Compounds 13A, 1A, heated at room temperature, and 1B. Compound 51B: LC-MS (ESI) m/z: 241 [M+H]⁺; ¹H-NMR (CDCl₃, 400 MHz): δ (ppm) 1.00 (t, J=7.2 Hz, 3H), 1.43-1.58 (m, 2H), 2.11-2.23 (m, 2H), 5.14-5.18 (m, 1H), 7.48-7.50 (m, 2H), 7.61-7.63 (m, 1H), 8.03 (d, J=7.6 Hz, 2H). Compound 51C: LC-MS (ESI) m/z: 332 [M+H]⁺; ¹H-NMR (CDCl₃, 400 MHz): δ (ppm) 0.89 (t, J=7.2 Hz, 3H), 1.33-1.49 (m, 2H), 1.61-1.70 (m, 1H), 1.91-1.99 (m, 1H), 4.68 (d, J=8.0 Hz, 1H), 5.01-5.06 (m, 1H), 6.56 (d, J=8.8 Hz, 2H), 7.24 (d, J=8.8 Hz, 2H), 7.50-7.54 (m, 2H), 7.61-7.65 (m, 1H), 7.99 (d, J=7.6 Hz, 2H). Compound 51: LC-MS (ESI) m/z: 529 [M+H]⁺; ¹H-NMR (Acetone-d₆, 400 MHz): δ (ppm) 0.46-0.69 (m, 3H), 0.99-1.07 (m, 1H), 1.25-1.28 (m, 1H), 1.67-1.75 (m, 1H), 1.89-1.99 (m, 1H), 4.40-4.52 (m, 1H), 6.43-6.77 (m, 1H), 7.26-7.41 (m, 8H), 7.51-7.58 (m, 3H), 7.63-7.75 (m, 2H).

Compound 51 was separated by chiral HPLC to give Compound 51-1 and Compound 51-2. Compound 51-1: LC-MS (ESI) m/z: 529 [M+H]⁺; ¹H-NMR (CDCl₃, 400 MHz): δ (ppm) 0.72 (t, J=7.2 Hz, 3H), 0.91-1.01 (m, 1H), 1.18-1.24 (m, 1H), 1.60-1.69 (m, 1H), 1.80-1.90 (m, 1H), 4.22-4.25 (m, 1H), 4.26 (s, 1H), 7.12 (d, J=8.8 Hz, 2H), 7.17 (d, J=8.8 Hz, 2H), 7.20 (d, J=8.8 Hz, 2H), 7.24-7.30 (m, 3H), 7.46-7.51 (m, 4H). Chiral separation condition: MeOH contained 0.2% Methanol Ammonia; (R,R)-Whelk-ol (4.6*250 mm, 5 μm); retention time: 3.21 minutes (90%), 4.13 minutes (10%). Compound 51-2: LC-MS (ESI) m/z: 529 [M+H]⁺; ¹H-NMR (CDCl₃, 400 MHz): δ (ppm) 0.72 (t, J=7.2 Hz, 3H), 0.87-1.01 (m, 1H), 1.18-1.27 (m, 1H), 1.62-1.67 (m, 1H), 1.81-1.80 (m, 1H), 4.15 (s, 1H), 4.23-4.26 (m, 1H), 7.12 (d, J=8.8 Hz, 2H), 7.17 (d, J=8.8 Hz, 2H), 7.21 (d, J=8.8 Hz, 2H), 7.26-7.31 (m, 3H), 7.47-7.51 (m, 4H). Chiral separation condition: MeOH contained 0.2% Methanol Ammonia; (R,R)-Whelk-ol (4.6*250 mm, 5 μm); retention time: 4.63 minutes (5%), 9.2 minutes (95%).

### Example 52

### Synthesis of 1,3-bis(4-bromophenyl)-4-hydroxy-4-(pyridin-3-yl)imidazolidin-2-one

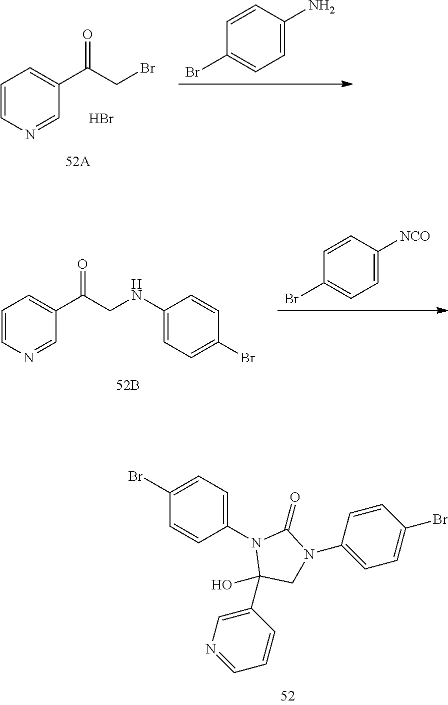

Compounds 52B and 52 were synthesized by employing the procedures described for Compounds 1B and 1 using Compounds 52A and 52B in lieu of Compounds 1A and 1B. Compound 52B: LC-MS (ESI) m/z: 291 [M+H]⁺. Compound 52: LC-MS (ESI) m/z: 488 [M+H]⁺; ¹H-NMR (DMSO-d₆, 400 MHz): δ (ppm) 4.05-4.24 (m, 2H), 7.30-7.45 (m, 5H), 7.54-7.67 (m, 4H), 7.91 (s, 1H), 7.95-8.00 (m, 1H), 8.44-8.48 (m, 1H), 8.78 (d, J=2.0 Hz, 1H).

### Example 53

### Synthesis of 1,3-bis(4-chlorophenyl)-4-hydroxy-5-methyl-4-phenylimidazolidin-2-one, (5S)-1,3-bis(4-chlorophenyl)-4-hydroxy-5-methyl-4-phenylimidazolidin-2-one, (5R)-1,3-bis(4-chlorophenyl)-4-hydroxy-5-methyl-4-phenylimidazolidin-2-one

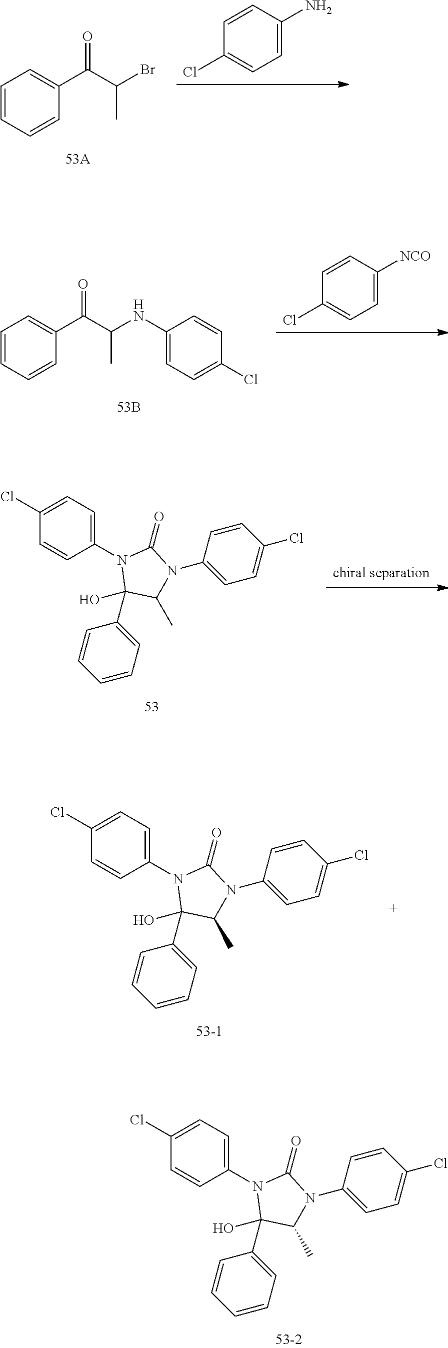

Compounds 53B and 53 were synthesized by employing the procedures described for Compounds 13C and 12 using Compounds 53A, 4-chloroaniline, and 53B in lieu of Compounds 13B, 4-bromoaniline, and 12B. Compound 53B: LC-MS (ESI) m/z: 260 [M+H]⁺; ¹H-NMR (CDCl₃, 400 MHz): δ (ppm) 1.47 (d, J=7.6 Hz, 3H), 4.73 (d, J=7.6 Hz, 1H), 5.04-5.11 (m, 1H), 6.59 (d, J=8.8 Hz, 2H), 7.12 (d, J=8.8 Hz, 2H), 7.52 (t, J=7.6 Hz, 2H), 7.62 (t, J=7.2 Hz, 1H), 7.80 (d, J=8.4 Hz, 2H). Compound 53: LC-MS: (ESI) m/z: 413 [M+H]⁺.

Compound 53 was separated by chiral HPLC to give Compound 53-1 and Compound 53-2. Compound 53-1: LC-MS (ESI) m/z: 413 [M+H]⁺; ¹H-NMR (DMSO-d₆, 400 MHz): δ (ppm) 1.08 (d, J=6.4 Hz, 3H), 4.32-4.37 (m, 1H), 7.23-7.27 (m, 3H), 7.30-7.34 (m, 3H), 7.43 (d, J=8 Hz, 6H), 7.57 (d, J=8.4 Hz, 2H). Chiral separation condition: MeOH; IC (4.6×250 mm, 5 μm); retention time: 1.67 minutes. Compound 53-2: LC-MS (ESI) m/z: 413 [M+H]⁺; ¹H-NMR (DMSO-d₆, 400 MHz): δ (ppm) 1.08 (d, J=6.4 Hz, 3H), 4.32-4.37 (m, 1H), 7.23-7.27 (m, 3H), 7.30-7.34 (m, 3H), 7.43 (d, J=8 Hz, 6H), 7.57 (d, J=7.2 Hz, 2H). Chiral separation condition: MeOH; IC (4.6×250 mm, 5 μm); retention time: 3.85 minutes.

### Example 54

### Synthesis of 1,3-bis(4-bromophenyl)-4-hydroxy-4-(6-methoxypyridin-3-yl)imidazolidin-2-one

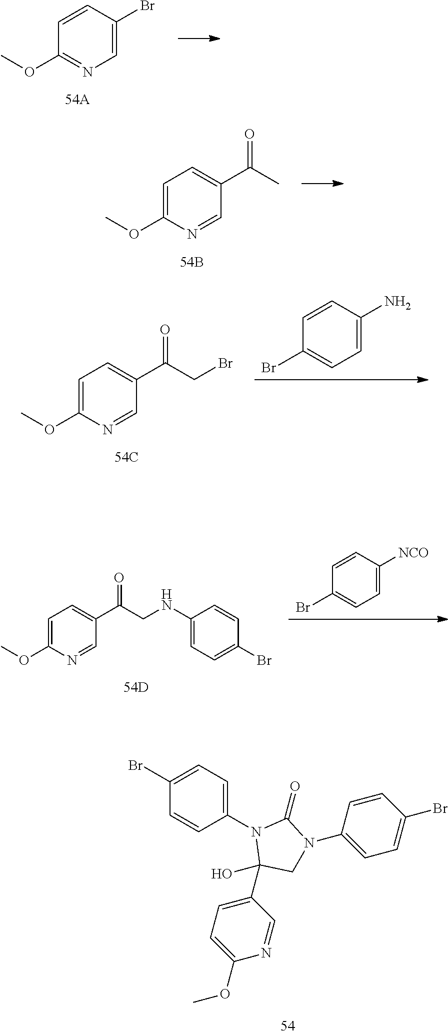

A mixture of tributyl(1-ethoxyvinyl)stannane (5.8 g, 16 mmol), 5-bromo-2-methoxypyridine 54A (2.0 g, 10.7 mmol), and dichlorobis(triphenylphosphine)palladium (100 mg, 0.12 mmol) in toluene (20 mL) was stirred at 100° C. under nitrogen for 18 hours. After cooled down to room temperature, the reaction mixture was quenched with hydrochloric acid (1 N, 50 mL) and extracted with ethyl acetate (50 mL×2). The combined organic layers was dried over sodium sulfate, filtered, and concentrated under reduced pressure. The residue was purified with flash column chromatography on silica gel (ethyl acetate in petroleum ether, 20% v/v) to furnish Compound 54B: LC-MS (ESI) m/z: 152 [M+H]⁺; ¹H-NMR (CDCl₃, 400 MHz): δ (ppm) 2.57 (s, 3H), 4.01 (s, 3H), 6.79 (d, J=8.8 Hz, 1H), 8.15 (d, J=9.2 Hz, 1H), 8.78 (s, 1H).

Compounds 54C, 54D, and 54 were synthesized by employing the procedures described for Compounds 13B, 1B, and 1 using Compounds 54B, 54C, and 54D in lieu of Compounds 13A, 1A, and 1B. Compound 54C: LC-MS (ESI) m/z: 230 [M+H]⁺; ¹H-NMR (CDCl₃, 400 MHz): δ (ppm) 4.03 (s, 3H), 4.37 (s, 2H), 6.82 (d, J=8.8 Hz, 1H), 8.16 (d, J=8.8 Hz, 1H), 8.84 (s, 1H). Compound 54D: LC-MS (ESI) m/z: 321 [M+H]⁺; ¹H-NMR (CDCl₃, 400 MHz): δ (ppm) 4.03 (s, 3H), 4.52 (d, J=4.4 Hz, 2H), 4.89 (s, 1H), 6.58 (d, J=8.8 Hz, 2H), 6.85 (d, J=8.8 Hz, 1H), 7.30 (d, J=8.8 Hz, 2H), 8.18 (d, J=10.8 Hz, 1H), 8.85 (s, 1H). Compound 54: LC-MS (ESI) m/z: 518 [M+H]⁺; ¹H-NMR (DMSO-d₆, 400 MHz): δ (ppm) 3.80 (s, 3H), 4.04 (d, J=10.8 Hz, 1H), 4.16 (d, J=10.8 Hz, 1H), 6.73 (d, J=8.8 Hz, 1H), 7.32-7.44 (m, 4H), 7.54-7.65 (m, 4H), 7.78-7.86 (m, 2H), 8.36 (m, 1H).

### Example 55

### Synthesis of 4-(3-bromophenyl)-1,3-bis(4-chlorophenyl)-4-hydroxyimidazolidin-2-one

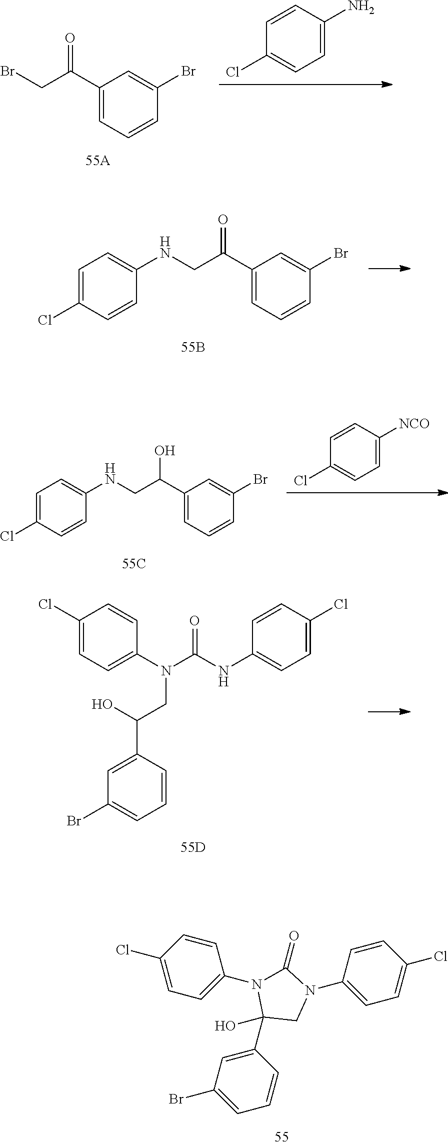

Compounds 55B, 55C, 55D, and 55 were synthesized by employing the procedures described for Compounds 1B, 14C, 1, and 14 using Compounds 55A, 4-chloroaniline, 55B, 55C, 1-chloro-4-isocyanatobenzene, and 55D in lieu of Compounds 1A, 4-bromoaniline, 1B, 1-bromo-4-isocyanatobenzene, and 14D. Compound 55B: LC-MS (ESI) m/z: 324 [M+H]⁺. Compound 55C: LC-MS (ESI) m/z: 326 [M+H]⁺. Compound 55D: LC-MS (ESI) m/z: 479 [M+H]⁺. Compound 55: LC-MS (ESI) m/z: 477 [M+H]⁺. ¹H-NMR (DMSO-d₆, 400 MHz): δ (ppm) 4.04 (d, J=10.8 Hz, 1H), 4.16 (d, J=10.4 Hz, 1H), 7.28-7.31 (m, 3H), 7.37-7.39 (m, 2H), 7.42-7.44 (m, 2H), 7.48 (s, 1H), 7.58 (d, J=8.0 Hz, 1H), 7.69 (d, J=8.8 Hz, 2H), 7.80 (s, 1H), 7.84 (s, 1H).

### Example 56

### Synthesis of 1,3-bis(4-chlorophenyl)-4-ethyl-4-hydroxyimidazolidin-2-one

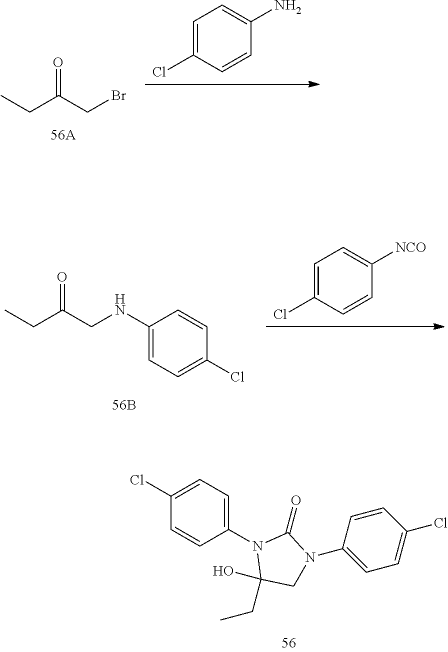

Compounds 56B and 56 were synthesized by employing the procedures described for Compounds 13C and 1 using Compounds 56A, 4-chloroaniline, 56B, and 1-chloro-4-isocyanatobenzene in lieu of Compounds 13B, 4-bromoaniline, 1B, and 1-bromo-4-isocyanatobenzene. Compound 56B: LC-MS (ESI) m/z: 198 [M+H]⁺. Compound 56: LC-MS (ESI) m/z: 351 [M+H]⁺; ¹H-NMR (DMSO-d₆, 400 MHz): δ (ppm) 0.94 (t, J=6.0 Hz, 3H), 1.80-1.95 (m, 2H), 3.84-4.18 (m, 2H), 6.0 (s, 1H), 7.36 (d, J=6.8 Hz, 2H), 7.43 (d, J=6.8 Hz, 2H), 7.59 (d, J=6.8 Hz, 2H), 7.72 (d, J=6.8 Hz, 2H).

### Example 57

### Synthesis of 1,3-bis(2,4-dichlorophenyl)-4-hydroxy-4-phenylimidazolidin-2-one

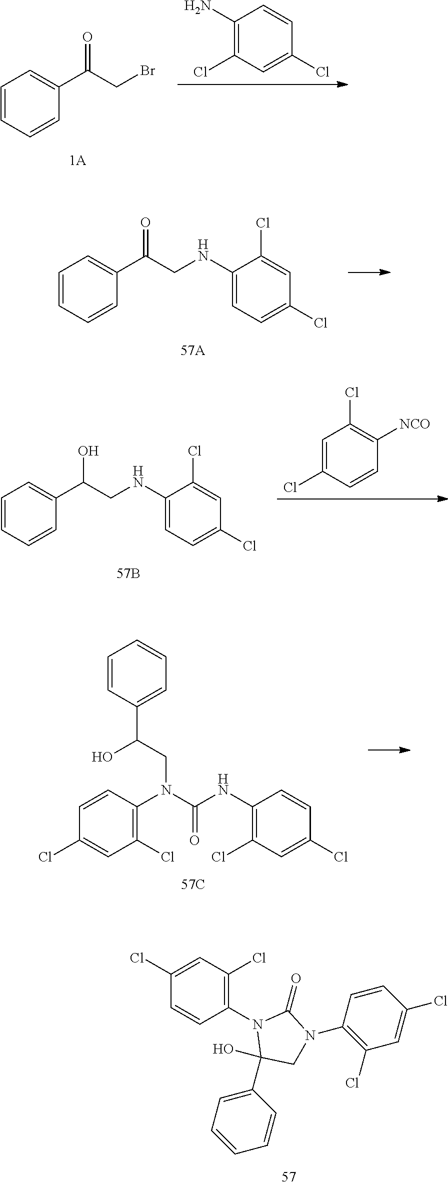

Compounds 57A, 57B, 57C, and 57 were synthesized by employing the procedures described for Compounds 1B, 14C, 1, and 14 using 2,4-dichloroaniline, Compounds 57A, 57B, 2,4-dichloro-1-isocyanatobenzene, and 57C in lieu of 4-bromoaniline, Compounds 14B, 1B, 1-bromo-4-isocyanatobenzene, and 14D. Compound 57A: LC-MS (ESI) m/z: 280 [M+H]⁺; ¹H-NMR (CDCl₃, 400 MHz): δ (ppm) 4.61 (s, 2H), 6.57 (d, J=8.4 Hz, 1H), 7.11-7.14 (m, 1H), 7.30 (s, 1H), 7.53 (t, J=8.0 Hz, 2H), 7.65 (t, J=7.2 Hz, 1H), 8.02 (d, J=8.0 Hz, 2H). Compound 57B: LC-MS (ESI) m/z: 282 [M+H]⁺. Compound 57C: LC-MS (ESI) m/z: 469 [M+H]⁺; ¹H-NMR (DMSO-d₆, 400 MHz): δ (ppm) 3.30 (s, 2H), 3.88-4.20 (m, 1H), 4.78-4.96 (m, 1H), 7.24-7.38 (m, 7H), 7.46 (s, 1H), 7.60 (s, 1H), 7.77-7.81 (m, 2H). Compound 57: LC-MS: (ESI) m/z: 467 [M+H]⁺; ¹H-NMR (DMSO-d₆, 400 MHz): δ (ppm) 3.94 (d, J=10 Hz, 1H), 4.29 (d, J=10 Hz, 1H), 7.21-7.36 (m, 4H), 7.54-7.79 (m, 8H).

### Example 58

### Synthesis of 1,3-bis(4-bromophenyl)-4-hydroxy-4-(3-(trifluoromethoxy)phenyl)imidazolidin-2-one

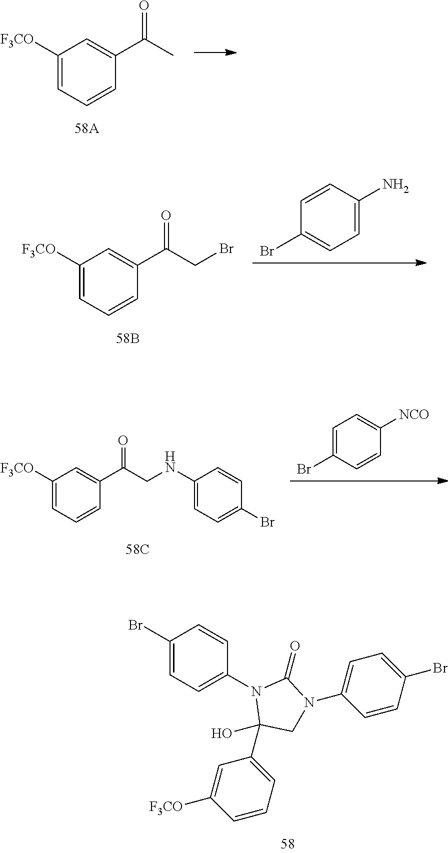

Compounds 58B, 58C, and 58 were synthesized by employing the procedures described for Compounds 13B, 1B, and 1 using Compounds 58A, 58B, and 58C in lieu of Compounds 13A, 1A, and 1B. Compound 58B: LC-MS (ESI) m/z: 283 [M+H]⁺; ¹H-NMR (CDCl₃, 400 MHz): δ (ppm) 4.43 (s, 2H), 7.46-7.58 (m, 2H), 7.84-7.93 (m, 2H). Compound 58C: LC-MS (ESI) m/z: 374 [M+H]⁺; ¹H-NMR (CDCl₃, 400 MHz): δ (ppm) 4.58 (d, J=4.4 Hz, 2H), 4.92 (s, 1H), 6.59 (d, J=8.8 Hz, 2H), 7.31 (d, J=8.8 Hz, 2H), 7.50 (d, J=7.6 Hz, 1H), 7.59 (t, J=8.0 Hz, 1H), 7.85 (s, 1H), 7.94 (d, J=7.6 Hz, 1H). Compound 58: LC-MS (ESI) m/z: 571 [M+H]⁺; ¹H-NMR (DMSO-d₆, 400 MHz): δ (ppm) 4.07 (d, J=10.8 Hz, 1H), 4.19 (d, J=10.4 Hz, 1H), 7.29-7.31 (m, 4H), 7.39-7.47 (m, 4H), 7.54-7.62 (m, 2H), 7.64-7.67 (m, 2H), 8.36 (m, 1H).

### Example 59

### Synthesis of 1,3-bis(4-bromophenyl)-4-hydroxy-4-(3-isopropylphenyl)imidazolidin-2-one

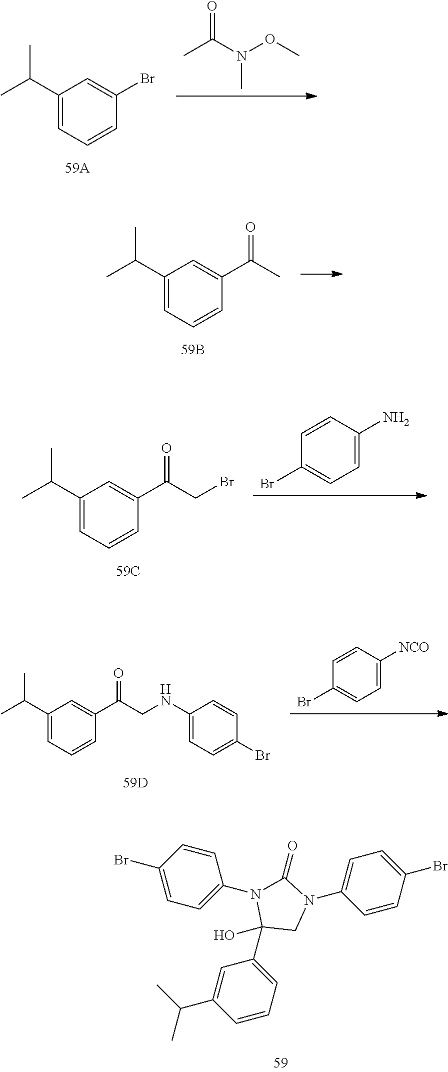

To a solution of Compound 59A (3.96 g, 20 mmol) in dry THF (20 mL) at −78° C. was dropped a solution of n-BuLi in THF (2.5 M, 8 mL, 18 mmol). After stirred at −78° C. for 1 hour, to the mixture was dropped N-methoxy-N-methylacetamide (1.87 g, 18 mmol). The mixture was stirred at −78° C. for 0.5 hour, quenched with water (10 mL), and extracted with ethyl acetate (20 mL×3). The combined organic layers was washed with brine (40 mL), dried over anhydrous sodium sulfate, filtered, and concentrated under reduced pressure. The residue was purified with flash column chromatography on silica gel (ethyl acetate in petroleum ether, from 0% to 8% v/v) to yield Compound 59B: LC-MS (ESI) m/z: 163 [M+H]⁺; ¹H-NMR (CDCl₃, 400 MHz): δ (ppm) 1.27 (d, J=7.2.4 Hz, 6H), 2.60 (s, 3H), 2.93-3.00 (m, 1H), 7.38 (t, J=7.6 Hz, 1H), 7.44 (t, J=7.2 Hz, 1H), 7.76 (d, J=7.2 Hz, 1H), 7.83 (s, 1H).

Compounds 59C, 59D, and 59 were synthesized by employing the procedures described for Compounds 13B, 1B, and 1 using Compounds 59B, 59C, and 59D in lieu of Compounds 13A, 1A and 1B. Compound 59C: LC-MS (ESI) m/z: 241 [M+H]⁺. Compound 59D: LC-MS (ESI) m/z: 332 [M+H]⁺. Compound 59: LC-MS: (ESI) m/z: 529 [M+H]⁺; ¹H-NMR (DMSO-d₆, 400 MHz): δ (ppm) 1.11 (d, J=8.4 Hz, 6H), 2.79-2.86 (m, 1H), 4.02 (d, J=10.8 Hz, 1H), 4.12 (d, J=10.8 Hz, 1H), 7.10-7.23 (m, 2H), 7.29-7.43 (m, 6H), 7.53 (d, J=8.4 Hz, 2H), 7.65 (t, J=8.4 Hz, 3H).

### Example 60

### Synthesis of 1,3-bis(4-chlorophenyl)-4-(3-ethynylphenyl)-4-hydroxyimidazolidin-2-one

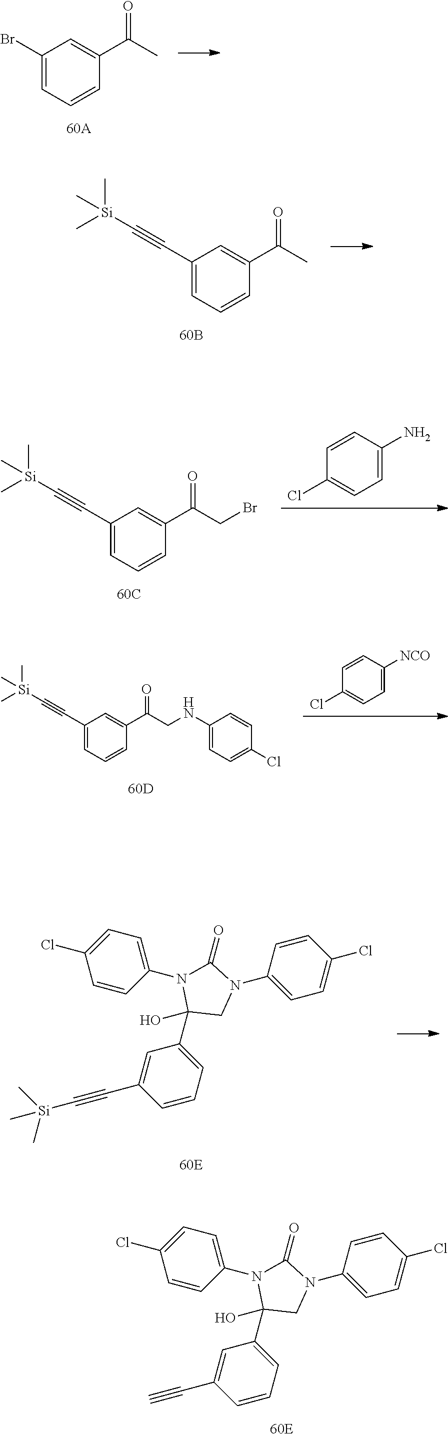

A mixture of 1-(3-bromophenyl)ethanone 60A (5.00 g, 25.12 mmol), ethynyltrimethylsilane (3.69 g, 37.69 mmol), PdCl₂(PPh₃)₂ (882 mg, 1.27 mmol), and CuI (119 mg, 0.63 mmol) in triethylamine (50 mL) was stirred at 50° C. under nitrogen for 16 hours. After cooled down to room temperature, the reaction mixture was diluted with ethyl acetate (160 mL) and filtered. The filtrate was concentrated under reduced pressure. The residue was purified with flash column chromatography on silica gel (ethyl acetate in petroleum ether, from 0% to 10% v/v) to give Compound 60B: LC-MS (ESI) m/z: 217 [M+H]⁺; ¹H-NMR (CDCl₃, 400 MHz): δ (ppm) 0.26 (s, 9H), 2.61 (s, 3H), 7.41 (t, J=8.0 Hz, 1H), 7.64 (d, J=8.4 Hz, 1H), 7.90 (d, J=8.0 Hz, 1H), 8.04 (s, 1H).

A mixture of Compound 60B (500 mg, 2.31 mmol) and CuBr₂ (1.03 g, 4.63 mmol) in ethyl acetate (10 mL) and dichloromethane (10 mL) was stirred at 70° C. for 5 hours After cooled down to room temperature, the reaction mixture was diluted with ethyl acetate (160 mL), washed with water (200 mL) and brine (200 mL), dried over anhydrous sulfate sodium, filtered, and concentrated under reduced pressure. The residue was purified with flash column chromatography on silica gel (ethyl acetate in petroleum ether, from 0% to 8% v/v) to furnish Compound 60C: LC-MS (ESI) m/z: non-ionizable compound under routine conditions used; ¹H-NMR (CDCl₃, 400 MHz): δ (ppm) 0.27 (s, 9H), 4.47 (s, 2H), 7.50 (t, J=8.0 Hz, 1H), 7.80 (d, J=8.4 Hz, 1H), 7.93 (d, J=8.0 Hz, 1H), 8.06 (s, 1H).

Compounds 60D and 60E were synthesized by employing the procedures described for Compounds 1B and 1 using Compounds 60C, 4-chloroaniline, 60D, and 1-chloro-4-isocyanatobenzene in lieu of Compounds 1A, 4-bromoaniline, 1B, and 1-bromo-4-isocyanatobenzene. Compound 60D: LC-MS (ESI) m/z: 342 [M+H]⁺; ¹H-NMR (CDCl₃, 400 MHz): δ (ppm) 0.28 (s, 9H), 4.57 (d, J=4.4 Hz, 2H), 4.92 (t, J=4.4 Hz, 1H), 5.24 (d, J=8.8 Hz, 2H), 7.17 (d, J=8.8 Hz, 2H), 7.47 (d, J=8.0 Hz, 1H), 7.70 (d, J=8.0 Hz, 1H), 7.95 (d, J=8.0 Hz, 1H), 8.07 (s, 1H). Compound 60E was used directly in next step without further purification. LC-MS (ESI) m/z: 495 [M+H]⁺.

A mixture of Compound 60E (33 mg, 0.07 mmol) and K₂CO₃ (29 mg, 0.21 mmol) in MeOH (2 mL) was stirred at 20° C. for 2 hours. The mixture was filtered and the filtrate was concentrated under reduced pressure. The residue was purified with preparative HPLC to afford Compound 60: LC-MS (ESI) m/z: 423 [M+H]⁺; ¹H-NMR (DMSO-d₆, 400 MHz): δ (ppm) 4.03 (d, J=10.8 Hz, 1H), 4.15 (d, J=10.8 Hz, 1H), 4.19 (s, 1H), 7.27-7.43 (m, 8H), 7.59-7.62 (m, 1H), 7.69-7.71 (m, 3H), 7.78 (s, 1H).

### Example 61

### Synthesis of 4-(1,3-bis(4-bromophenyl)-4-hydroxy-2-oxoimidazolidin-4-yl)benzonitrile

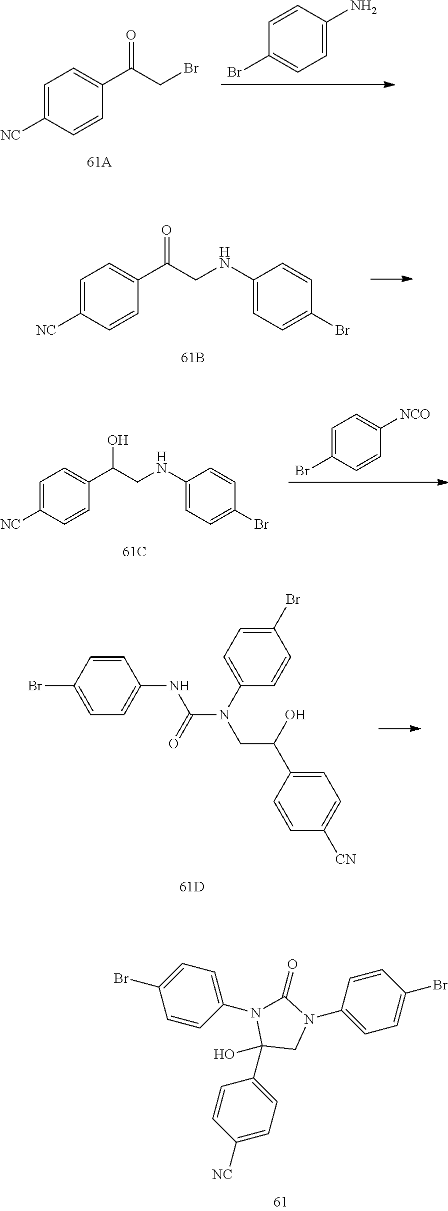

Compounds 61B, 61C, 61D, and 61 were synthesized by employing the procedures described for Compounds 1B, 14C, 1, and 14 using Compounds 61A, 61B, 61C, and 61D in lieu of Compounds 1A, 14B, 1B, and 14D. Compound 61B: LC-MS (ESI) m/z: 315 [M+1]⁺. Compound 61C: LC-MS (ESI) m/z: 317 [M+1]⁺. Compound 61D: LC-MS (ESI) m/z: 514 [M+1]⁺. Compound 61: LC-MS (ESI) m/z: 512 [M+H]⁺; ¹H-NMR (DMSO-d₆, 400 MHz): δ (ppm) 4.05-4.17 (m, 2H), 7.30-7.42 (m, 4H), 7.54-7.66 (m, 4H), 7.80 (s, 4H), 7.96 (s, 1H).

### Example 62

### Synthesis of 4-(3-chlorophenyl)-1,3-bis(4-chlorophenyl)-4-hydroxy-5-methylimidazolidin-2-one, (5S)-4-(3-chlorophenyl)-1,3-bis(4-chlorophenyl)-4-hydroxy-5-methylimidazolidin-2-one, and (5R)-4-(3-chlorophenyl)-1,3-bis(4-chlorophenyl)-4-hydroxy-5-methylimidazolidin-2-one

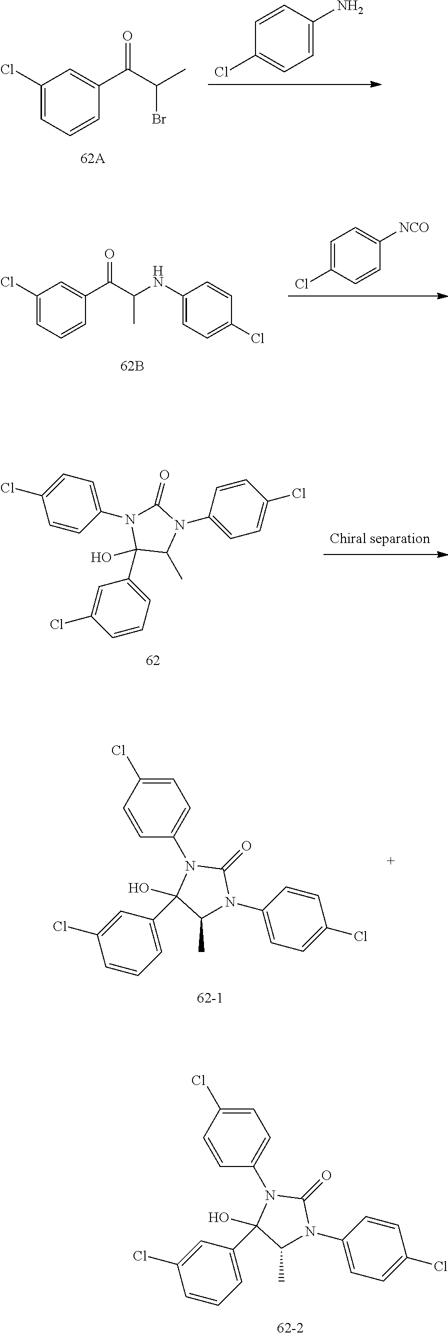

Compounds 62B and 62 were synthesized by employing the procedures described for Compounds 1B and 1 using Compounds 62A, 4-chloroaniline, 62B, and 1-chloro-4-isocyanatobenzene in lieu of Compounds 1A, 4-bromoaniline, 1B, and 1-bromo-4-isocyanatobenzene. Compound 62B: LC-MS (ESI) m/z: 294 [M+H]⁺.

Compound 62 was separated by chiral HPLC to give Compound 62-1 and Compound 62-2. Compound 62-1: LC-MS: (ESI) m/z: 447 [M+H]⁺; ¹H-NMR (DMSO-d₆, 400 MHz): δ (ppm) 1.29 (d, J=5.2 Hz, 3H), 4.52-4.53 (m, 1H), 6.94-7.77 (m, 12H); Chiral separation condition: MeOH contained 0.5% NH₄OH); OZ-H (4.6*250 mm, 5 μm); retention time: 1.86 minutes. Compound 62-2: LC-MS: (ESI) m/z: 447 [M+H]⁺; ¹H-NMR (DMSO-d₆, 400 MHz): δ (ppm) 1.29 (d, J=5.2 Hz, 3H), 4.52-4.53 (m, 1H), 6.94-7.77 (m, 12H). Chiral separation condition: MeOH contained 0.5% NH₄OH); OZ-H (4.6*250 mm, 5 μm); retention time: 3.08 minutes.

### Example 63

### Synthesis of 3-(1,3-bis(4-chlorophenyl)-4-hydroxy-2-oxoimidazolidin-4-yl)benzonitrile

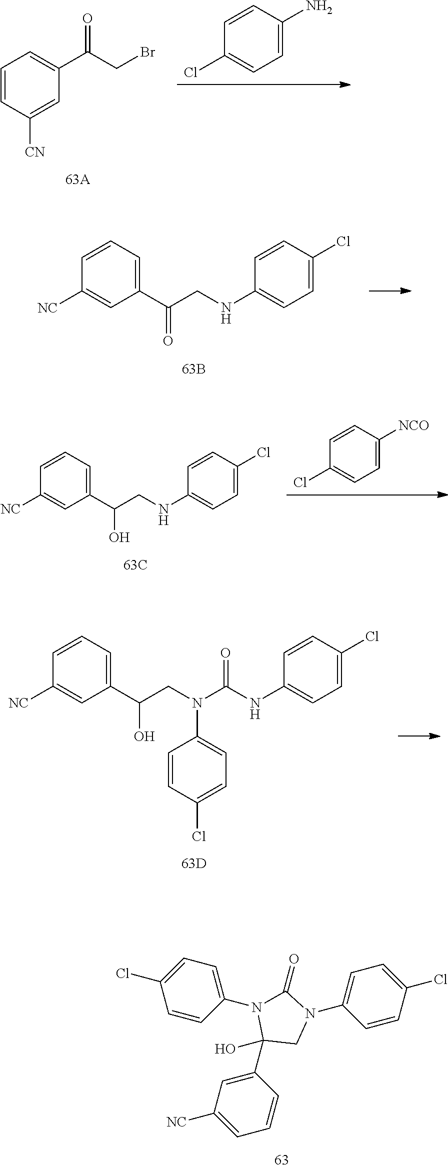

Compounds 63B, 63C, 63D, and 63 were synthesized by employing the procedures described for Compounds 1B, 14C, 1, and 14 using Compounds 63A, 4-chloroaniline, 63B, 63C, 1-chloro-4-isocyanatobenzene, and 63D in lieu of Compounds 1A, 4-bromoaniline, 14B, 1B, 1-bromo-4-isocyanatobenzene, and 14D. Compound 63B: LC-MS (ESI) m/z: 271 [M+H]⁺. Compound 63C: LC-MS (ESI) m/z: 273 [M+H]⁺; ¹H-NMR (CDCl₃, 400 MHz): δ (ppm) 3.20-3.26 (m, 1H), 3.37-3.41 (m, 1H), 4.93-4.96 (m, 1H), 6.57-6.61 (m, 2H), 7.12-7.16 (m, 2H), 7.47-7.51 (m, 1H), 7.59-7.65 (m, 2H), 7.72-7.73 (m, 1H). Compound 63D: LC-MS (ESI) m/z: 426 [M+H]⁺. Compound 63: LC-MS: (ESI) m/z: 424 [M+H]⁺. ¹H-NMR (DMSO-d₆, 400 MHz): δ (ppm) 4.06 (d, J=10.8 Hz, 1H), 4.21 (d, J=10.8 Hz, 1H), 7.27-7.30 (m, 2H), 7.36-7.38 (m, 2H), 7.42-7.44 (m, 2H), 7.53-7.55 (m, 1H), 7.69-7.75 (m, 3H), 7.93-7.95 (m, 2H), 8.07-8.08 (m, 1H).

### Example 64

### Synthesis of 1,3-bis(4-chlorophenyl)-4-(3-fluorophenyl)-4-hydroxyimidazolidin-2-one

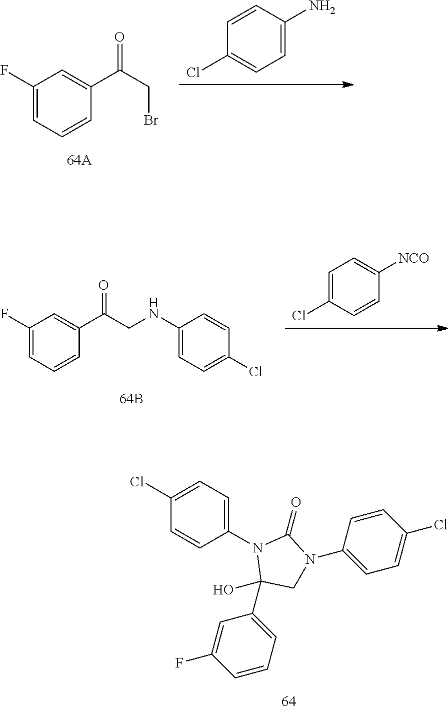

Compounds 64B and 64 were synthesized by employing the procedures described for Compounds 1B and 1 using Compounds 64A, 4-chloroaniline, 64B, and 1-chloro-4-isocyanatobenzene in lieu of Compounds 1A, 4-bromoaniline, 1B, and 1-bromo-4-isocyanatobenzene. Compound 64B: LC-MS (ESI) m/z: 264 [M+1]⁺. Compound 64: LC-MS (ESI) m/z: 417 [M+H]⁺; ¹H-NMR (DMSO-d₆, 400 MHz): δ (ppm) 4.04-4.19 (m, 2H), 7.07-7.15 (m, 1H), 7.27-7.44 (m, 8H), 7.61-7.65 (m, 1H), 7.70-7.73 (m, 2H), 7.81 (s, 1H).

### Example 65

### Synthesis of 1,3-bis(4-chlorophenyl)-4-cyclopropyl-5-(3-(trifluoromethoxy)phenyl)-1,3-dihydro-2H-imidazol-2-one

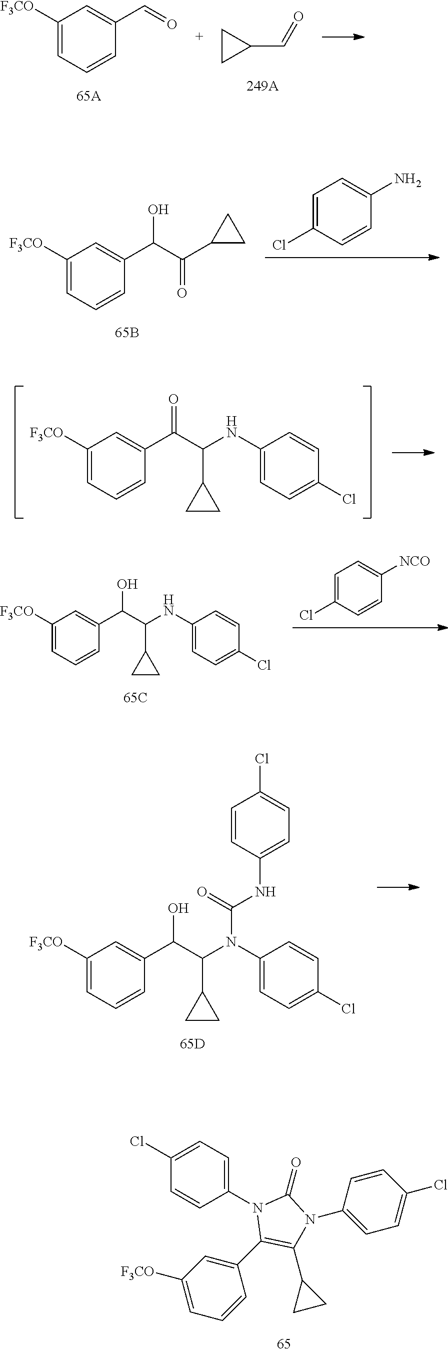

Compounds 65B, 65C, 65D, and 65 were synthesized by employing the procedures described for Compounds 249C, 48B, 1, and 18 using Compounds 65A, 65B, 65C, 1-chloro-4-isocyanatobenzene, and Compound 65D in lieu of Compounds 249B, 48A, 1B, 1-bromo-4-isocyanatobenzene, and Compound 18C. Compound 65B. LC-MS (ESI) m/z: 243 [M-OH]⁺. Compound 65C. LC-MS (ESI) m/z: 372 [M+H]⁺; ¹H-NMR (CDCl₃, 400 MHz): δ (ppm) −0.06-0.00 (m, 1H), 0.13-0.16 (m, 1H), 0.31-0.45 (m, 2H), 0.82-0.88 (m, 1H), 2.64 (d, J=4.8 Hz, 1H), 2.90-2.92 (m, 1H), 3.80 (brs, 1H), 5.04 (t, J=4.0 Hz, 1H), 6.62 (d, J=8.8 Hz, 2H), 7.13-7.17 (m, 3H), 7.28-7.40 (m, 3H). Compound 65D. LC-MS (ESI) m/z: 525 [M+H]⁺. Compound 65. LC-MS (ESI) m/z: 505 [M+H]⁺; ¹H-NMR (CDCl₃, 400 MHz): δ (ppm) 0.005-0.007 (m, 2H), 0.61-0.63 (m, 2H), 1.66-1.70 (m, 1H), 6.98 (d, J=7.6 Hz, 1H), 7.08-7.13 (m, 4H), 7.26-7.30 (m, 3H), 7.43-7.47 (m, 4H).

### Example 66

### Synthesis of 1,3-bis(4-bromophenyl)-4-hydroxy-5-methyl-4-(3-(trifluoromethoxy)phenyl)imidazolidin-2-one, (5S)-1,3-bis(4-bromophenyl)-4-hydroxy-5-methyl-4-(3-(trifluoromethoxy)phenyl)imidazolidin-2-one, and (5R)-1,3-bis(4-bromophenyl)-4-hydroxy-5-methyl-4-(3-(trifluoromethoxy)phenyl)imidazolidin-2-one

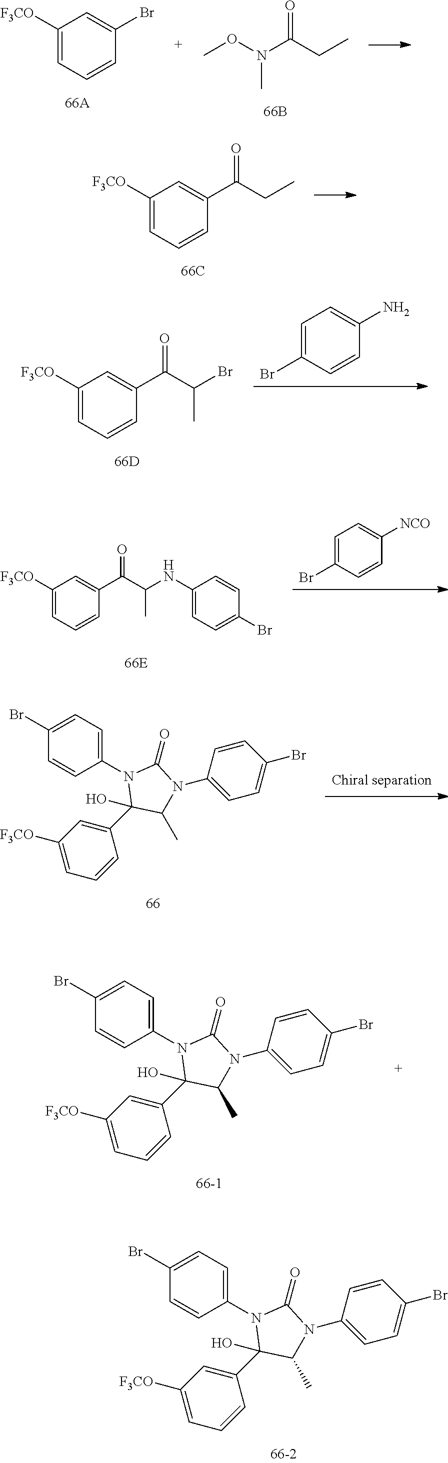

Compounds 66C, 66D, 66E, and 66 were synthesized by employing the procedures described for Compounds 59B, 13B, 13C, and 1 using Compounds 66A, 66B, 66C, 66D, and 66E in lieu of Compounds 59A, N-methoxy-N-methylacetamide, 13A, 13B, and 1B. Compound 66C: LC-MS (ESI) m/z: 219 [M+H]⁺. Compound 66D: LC-MS (ESI) m/z: 297 [M+H]⁺. Compound 66E: LC-MS (ESI) m/z: 388 [M+H]⁺; ¹H-NMR (CDCl₃, 400 MHz): δ (ppm) 1.48 (d, J=6.4 Hz, 3H), 4.67 (s, 1H), 5.02 (s, 1H), 6.54 (d, J=8.8 Hz, 2H), 7.25-7.28 (m, 2H), 7.47-7.49 (m, 1H), 7.57 (t, J=8.0 Hz, 1H), 7.84 (s, 1H), 7.93 (d, J=8.0 Hz, 1H).

Compound 66: LC-MS (ESI) m/z: 585 [M+H]⁺; ¹H-NMR (DMSO-d₆, 400 MHz): δ (ppm) 0.65-1.08 (dd, J=6.8 Hz, 3H), 4.37-4.56 (m, 1H), 7.25-7.53 (m, 8H), 7.56-7.77 (m, 5H) was purified with chiral HPLC to give Compound 66-1 and Compound 66-2. Compound 66-1: LC-MS (ESI) m/z: 585 [M+H]⁺; ¹H-NMR (DMSO-d₆, 400 MHz): δ (ppm) 0.65-1.08 (dd, J=6.8 Hz, 3H), 4.37-4.57 (m, 1H), 7.25-7.28 (m, 1H), 7.33-7.64 (m, 12H); Chiral separation condition: MeOH contained 0.2% Methanol ammonia; RegisCell (250*4.6 mm, 5 μm); retention time: 1.19 minutes (79%), 2.95 minutes (21%). Compound 66-2: LC-MS (ESI) m/z: 585 [M+H]⁺; ¹H-NMR (DMSO-d₆, 400 MHz): δ (ppm) 0.65-1.09 (dd, J=6.8 Hz, 3H), 4.38-4.57 (m, 1H), 7.25-7.28 (m, 1H), 7.33-7.90 (m, 12H); Chiral separation condition: co-solvents: MeOH contained 0.2% Methanol ammonia; RegisCell (250*4.6 mm, 5 μm); retention time: 2.68 minutes (19%), 4.31 minutes (81%).

### Example 67

### Synthesis of 1,3-bis(4-bromophenyl)-5-ethyl-4-hydroxy-4-(3-(trifluoromethyl)phenyl)imidazolidin-2-one, (5S)-1,3-bis(4-bromophenyl)-5-ethyl-4-hydroxy-4-(3-(trifluoromethyl)phenyl)imidazolidin-2-one, and (5R)-1,3-bis(4-bromophenyl)-5-ethyl-4-hydroxy-4-(3-(trifluoromethyl)phenyl)imidazolidin-2-one

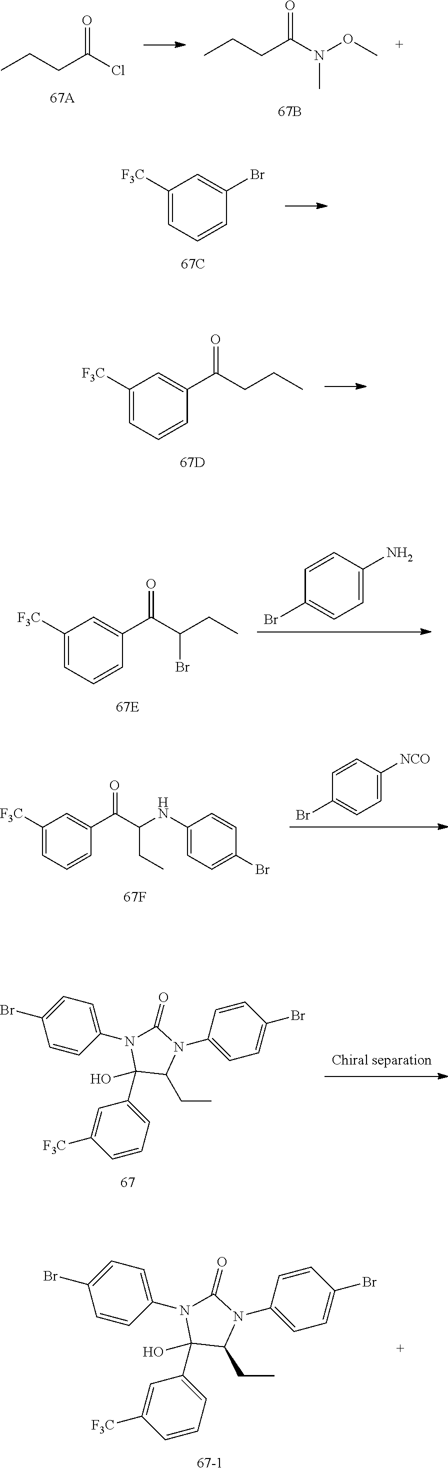

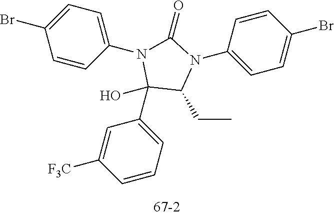 [SMILES: CC[C@H]1N(c2ccc(Br)cc2)C(=O)N(c2ccc(Br)cc2)C1(O)c1cccc(C(F)(F)F)c1]

Compounds 67B, 67D, 67E, 67F, and 67 were synthesized by employing the procedures described for Compounds 42B, 59B, 13B, 13C, and 1 using Compounds 67A, 67B, 67C, 67D, 67E, and 67F in lieu of Compounds 42A, 59A, N-methoxy-N-methylacetamide, 13A, 13B, and 1B. Compound 67B: LC-MS (ESI) m/z: 132 [M+H]⁺; ¹H-NMR (CDCl₃, 400 MHz): δ (ppm) 0.97 (t, J=7.2 Hz, 3H), 1.62-1.71 (m, 2H), 2.40 (t, J=7.2 Hz, 2H), 3.18 (s, 3H), 3.69 (s, 3H). Compound 67D. ¹H-NMR (CDCl₃, 400 MHz): δ (ppm) 0.95 (t, J=7.2 Hz, 3H), 1.69-1.74 (m, 2H), 2.91 (t, J=7.2 Hz, 2H), 7.53 (t, J=8.4 Hz, 1H), 7.74 (d, J=7.6 Hz, 1H), 8.07 (d, J=7.6 Hz, 1H), 8.14 (s, 1H). Compound 67E: LC-MS (ESI) m/z: 295 [M+H]⁺; ¹H-NMR (CDCl₃, 400 MHz): δ (ppm) 1.03 (t, J=7.6 Hz, 3H), 2.07-2.21 (m, 2H), 4.96 (q, J=6.4 Hz, 1H), 7.57 (t, J=8.4 Hz, 1H), 7.78 (d, J=7.6 Hz, 1H), 8.12 (d, J=7.6 Hz, 1H), 8.20 (s, 1H). Compound 67F: LC-MS (ESI) m/z: 386 [M+H]⁺; ¹H-NMR (CDCl₃, 400 MHz): δ (ppm) 0.82 (t, J=7.2 Hz, 3H), 1.66 (m, 1H), 2.02 (m, 1H), 4.62 (d, J=8.0 Hz, 1H), 4.94 (m, 1H), 6.50 (d, J=8.8 Hz, 2H), 7.18 (d, J=8.8 Hz, 2H), 7.59 (t, J=7.6 Hz, 1H), 7.80 (d, J=8.0 Hz, 1H), 8.09 (d, J=8.0 Hz, 1H), 8.15 (s, 1H). Compound 67: LC-MS (ESI) m/z: 583 [M+H]⁺; ¹H-NMR (CDCl₃, 400 MHz): δ (ppm) 0.31-0.70 (dt, J 7.6 Hz, 3H), 1.80-1.82 (m, 2H), 3.99 (s, 1H), 4.14-4.17 (m, 1H), 7.17-7.25 (m, 6H), 7.45-7.68 (m, 4H), 7.68-7.88 (m, 2H).

Compound 67 was purified with chiral HPLC to give Compound 67-1 and Compound 67-2. Compound 67-1: LC-MS (ESI) m/z: 583 [M+H]⁺; ¹H-NMR (CDCl₃, 400 MHz): δ (ppm) 0.29-0.72 (dt, J=7.6 Hz, 3H), 1.84-1.86 (m, 2H), 3.94 (s, 1H), 4.15-4.17 (m, 1H), 7.17-7.25 (m, 5H), 7.45-7.68 (m, 5H), 7.68-7.88 (m, 2H). Chiral separation condition: MeOH contained 0.2% Methanol Ammonia; OD-H (4.6*150 mm, 5 μm); retention time: 1.46 minutes (85%), 3.15 minutes (15%). Compound 67-2: LC-MS (ESI) m/z: 583 [M+H]⁺; ¹H-NMR (CDCl₃, 400 MHz): δ (ppm) 0.29-0.72 (dt, J=7.6 Hz, 3H), 1.82-1.87 (m, 2H), 4.06 (s, 1H), 4.14-4.17 (m, 1H), 7.15-7.26 (m, 5H), 7.33-7.57 (m, 5H), 7.67-7.88 (m, 2H). Chiral separation condition: MeOH contained 0.2% Methanol Ammonia; OD-H (4.6*150 mm, 5 μm); retention time: 2.43 minutes (30%), 4.31 minutes (70%).

### Example 68

### Synthesis of 1,3-bis(4-bromophenyl)-4-(3-ethylphenyl)-4-hydroxy-5-methylimidazolidin-2-one, (5S)-1,3-bis(4-bromophenyl)-4-(3-ethylphenyl)-4-hydroxy-5-methylimidazolidin-2-one, and (5R)-1,3-bis(4-bromophenyl)-4-(3-ethylphenyl)-4-hydroxy-5-methylimidazolidin-2-one

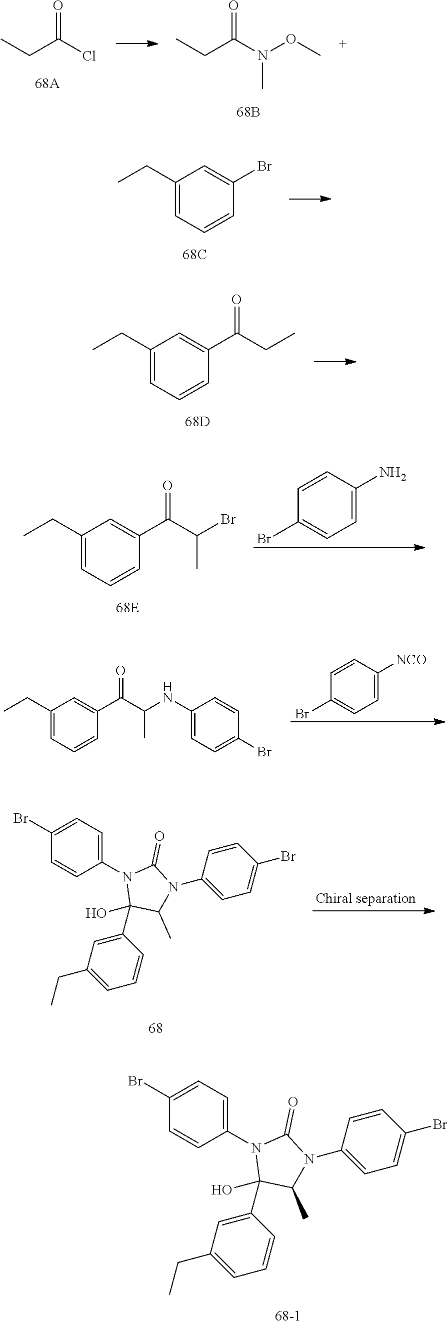

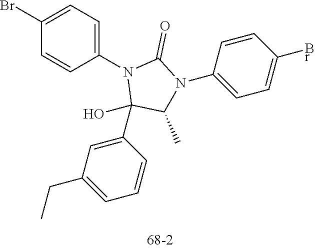 [SMILES: CCc1cccc(C2(O)[C@@H](C)N(c3ccc(Br)cc3)C(=O)N2c2ccc(Br)cc2)c1]

Compounds 68B, 68D, 68E, 68F, and 67 were synthesized by employing the procedures described for Compounds 42B, 59B, 13B, 1B, and 1 using Compounds 68A, 68B, 68C, 68D, 68E, heated at 60° C., and 68F in lieu of Compounds 42A, 59A, N-methoxy-N-methylacetamide, 13A, 1A, stirred at room temperature, and 1B.

Compound 68B: LC-MS (ESI) m/z: 118 [M+H]⁺; ¹H-NMR (CDCl₃, 400 MHz): δ (ppm) 1.12-1.16 (m, 3H), 2.42-2.48 (m, 2H), 3.18 (s, 3H), 3.68 (s, 3H). Compound 68D: LC-MS (ESI) m/z: non-ionizable compound under routine conditions used; ¹H-NMR (CDCl₃, 400 MHz): δ (ppm) 1.21-1.28 (m, 6H), 2.71 (t, J=8.0 Hz, 2H), 3.00 (t, J=11.2 Hz, 2H), 7.35-7.41 (m, 2H), 7.77-7.80 (m, 2H). Compound 68E: LC-MS (ESI) m/z: 241 [M+H]⁺; ¹H-NMR (CDCl₃, 400 MHz): δ (ppm) 1.27 (t, J=7.2 Hz, 3H), 1.90 (d, J=6.4 Hz, 3H), 2.72 (q, J=7.6 Hz, 2H), 5.30 (q, J=6.8 Hz, 1H), 7.38-7.45 (m, 2H), 7.83-7.86 (m, 2H). Compound 68F: LC-MS (ESI) m/z: 332 [M+H]⁺; ¹H-NMR (CDCl₃, 400 MHz): δ (ppm) 1.28 (t, J=7.6 Hz, 3H), 1.46 (d, J=6.8 Hz, 3H), 2.73 (q, J=7.2 Hz, 2H), 4.76 (d, J=7.6 Hz, 1H), 5.03-5.10 (m, 1H), 6.53-6.57 (m, 2H), 7.23-7.26 (m, 2H), 7.41-7.47 (m, 2H), 7.79-7.82 (m, 2H). Compound 68: LC-MS (ESI) m/z: 529 [M+H]⁺; ¹H-NMR (CDCl₃, 400 MHz): δ (ppm) 0.67-1.23 (dt, J=6.8 Hz, 3H), 1.10-1.18 (m, 3H), 2.57-2.64 (m, 2H), 3.74, 4.59 (s, 1H), 4.20, 4.29 (q, J=6.4 Hz, 1H), 7.11-7.48 (m, 12H).

Compound 68 was purified with chiral HPLC to yield Compound 68-1 and Compound 68-2. Compound 68-1: LC-MS (ESI) m/z: 529 [M+H]⁺; ¹H-NMR (CDCl₃, 400 MHz): δ (ppm) 1.16 (t, J=7.6 Hz, 3H), 1.26 (d, J=6.4 Hz, 3H), 2.60 (q, J=7.2 Hz, 2H), 3.71 (s, 1H), 4.23 (q, J=7.2 Hz, 1H), 7.11-7.32 (m, 10H), 7.47-7.49 (m, 2H). Chiral separation condition: MeOH contained 0.2% methanol ammonia; RegisCell (4.6*250 mm, 5 μm); retention time: 2.07 minutes (78.9%), 3.05 minutes (21.1%). Compound 68-2: LC-MS (ESI) m/z: 529 [M+H]⁺; ¹H-NMR (CDCl₃, 400 MHz): δ (ppm) 0.63-1.24 (dt, J=6.8 Hz, 3H), 1.09-1.17 (m, 3H), 2.55-2.63 (m, 2H), 4.01 (s, 1H), 4.18, 4.23 (q, J=6.4 Hz, 1H), 7.07-7.38 (m, 10H), 7.36-7.46 (m, 2H). Chiral separation condition MeOH contained 0.2% methanol ammonia; RegisCell (4.6*250 mm, 5 m); retention time: 2.7 minutes (23.8%), 4.96 minutes (760.1%).

### Example 69

### Synthesis of 1,3-bis(4-chlorophenyl)-4-(3-ethylphenyl)-4-hydroxy-5-methylimidazolidin-2-one

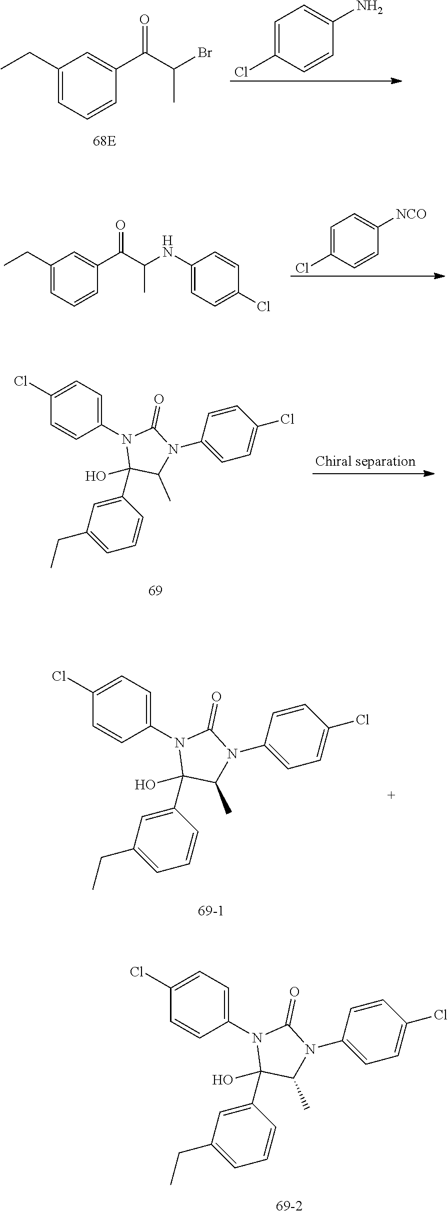

Compounds 69A and 69 were synthesized by employing the procedures described for Compounds 1B and 1 using Compounds 68E, 4-chloroaniline, heated at 60° C., 69A, and 1-chloro-4-isocyanatobenzene in lieu of Compounds 1A, 4-bromoaniline, stirred at room temperature, 1B, and 1-bromo-4-isocyanatobenzene. Compound 69A: LC-MS (ESI) m/z: 288 [M+H]⁺; ¹H-NMR (CDCl₃, 400 MHz): δ (ppm) 1.28 (t, J=7.6 Hz, 3H), 1.46 (d, J=6.8 Hz, 3H), 2.73 (q, J=7.6 Hz, 2H), 4.74 (d, J=5.6 Hz, 1H), 5.03-5.10 (m, 1H), 6.58-6.61 (m, 2H), 7.09-7.14 (m, 2H), 7.40-7.47 (m, 2H), 7.80-7.82 (m, 2H). Compound 69: LC-MS (ESI) m/z: 441 [M+H]⁺; ¹H-NMR (CDCl₃, 400 MHz): δ (ppm) 0.70-1.25 (dd, J=6.8 Hz, 3H), 1.11-1.18 (m, 3H), 2.58-2.64 (m, 2H), 3.64 (s, 1H), 4.22, 4.30 (q, J=6.4 Hz, 1H), 7.10-7.43 (m, 12H).

Compound 69 was separated with chiral HPLC to yield Compound 69-1 and Compound 69-2. Compound 69-1: LC-MS (ESI) m/z: 441 [M+H]⁺; ¹H-NMR (CDCl₃, 400 MHz): δ (ppm) 0.68-1.25 (d, J=6.8 Hz, 3H), 1.11-1.18 (m, 3H), 2.58-2.64 (m, 2H), 3.66 (s, 1H), 4.21, 4.30 (q, J=6.4 Hz, 1H), 7.10-7.40 (m, 12H). Chiral separation condition: MeOH contained 0.2% methanol ammonia; RegisCell (4.6*250 mm, 5 m); retention time: 1.98 minutes (76.7%), 2.98 minutes (23.3%). Compound 69-2: LC-MS (ESI) m/z: 441 [M+H]⁺; ¹H-NMR (CDCl₃, 400 MHz): δ (ppm) 0.70-1.26 (d, J=6.8 Hz, 3H), 1.16, 1.13 (t, J=7.2 Hz, 3H), 2.58-2.64 (m, 2H), 3.51 (s, 1H), 4.22, 4.23 (q, J=6.4 Hz, 1H), 7.11-7.43 (m, 12H). Chiral separation condition: MeOH contained 0.2% methanol ammonia; RegisCell (4.6*250 mm, 5 μm); retention time: 2.79 minutes (23.3%), 4.96 minutes (76.2%).

### Example 70

### Synthesis of 1,3-bis(4-chlorophenyl)-4-cyclopropyl-5-(3-(difluoromethoxy)phenyl)-1,3-dihydro-2H-imidazol-2-one

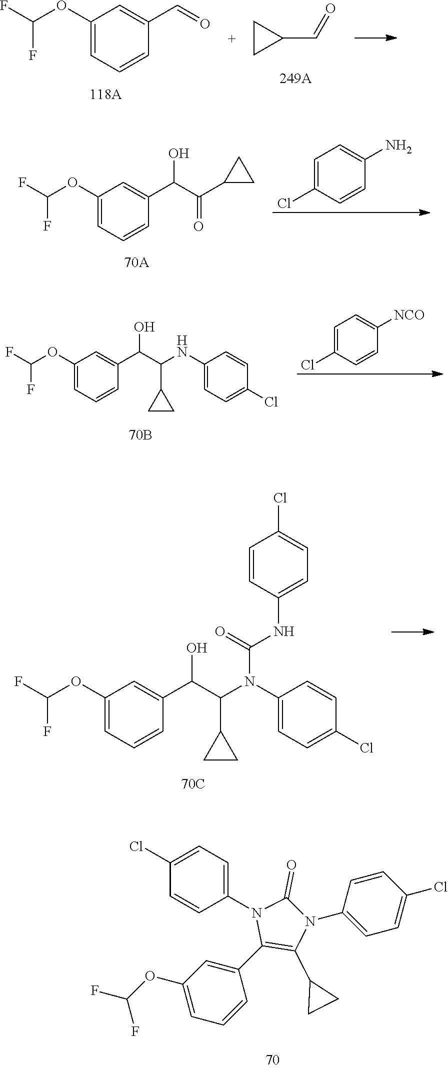

Compounds 70A, 70B, 70C, and 70 were synthesized by employing the procedures described for Compounds 249C, 48B, 1, and 14 using Compounds 118A, 70A, 70B, 1-chloro-4-isocyanatobenzene, and Compound 70C in lieu of Compounds 249B, 48A, 1B, 1-bromo-4-isocyanatobenzene, and Compound 14D. Compound 70A. LC-MS (ESI) m/z: 225 [M-OH]⁺. Compound 70B. LC-MS (ESI) m/z: 354 [M+H]⁺; ¹H-NMR (CDCl₃, 400 MHz): δ (ppm) −0.11-−0.07 (m, 1H), 0.15-0.17 (m, 1H), 0.35-0.43 (m, 2H), 0.86-0.88 (m, 1H), 2.63-2.64 (m, 1H), 2.93-2.94 (m, 1H), 3.78-3.80 (m, 1H), 5.02 (s, 1H), 6.34-6.71 (m, 4H), 7.05-7.07 (m, 1H), 7.12-7.15 (m, 2H), 7.24 (s, 1H), 7.34-7.36 (m, 1H). Compound 70C. LC-MS (ESI) m/z: 507 [M+H]⁺; ¹H-NMR (CDCl₃, 400 MHz): δ (ppm) 0.02-0.04 (m, 1H), 0.22-0.27 (m, 1H), 0.37-0.42 (m, 1H), 0.51-0.57 (m, 1H), 0.96-1.00 (m, 1H), 3.50 (d, J=10.4 Hz, 1H), 4.47 (d, J=4.0 Hz, 1H), 5.33 (s, 1H), 5.89 (s, 1H), 6.32-6.6.69 (m, 1H), 6.98-7.07 (m, 3H), 7.18-7.24 (m, 4H), 7.29-7.37 (m, 5H). Compound 70. LC-MS (ESI) m/z: 487 [M+H]⁺; ¹H-NMR (CD₃OD, 400 MHz): δ (ppm) 0.00-0.02 (m, 2H), 0.52-0.53 (m, 2H), 1.73-1.78 (m, 1H), 6.68 (t, J=73.6 Hz, 1H), 6.96-7.02 (m, 3H), 7.13-7.15 (m, 2H), 7.24-7.33 (m, 3H), 7.50 (s, 4H).

### Example 71

### Synthesis of 1,3-bis(4-bromophenyl)-4-hydroxy-4-(3-methoxyphenyl)-5-methylimidazolidin-2-one, (5S)-1,3-bis(4-bromophenyl)-4-hydroxy-4-(3-methoxyphenyl)-5-methylimidazolidin-2-one, and (5R)-1,3-bis(4-bromophenyl)-4-hydroxy-4-(3-methoxyphenyl)-5-methylimidazolidin-2-one

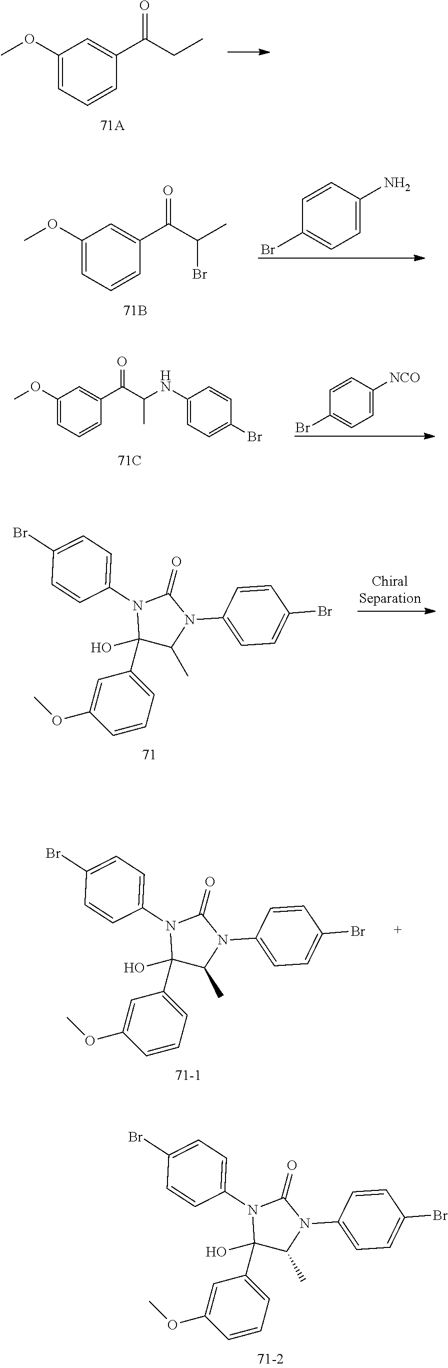

Compounds 71B, 71C, and 71 were synthesized by employing the procedures described for Compounds 13B, 13C, and 1 using Compounds 71A, using AcOH as solvent, 71B, using EtOH as solvent, and 71C in lieu of Compounds 13A, using diethyl ether as solvent, 13B, using THF as solvent, and 1B. Compound 71B: LC-MS (ESI) m/z: 243 [M+H]⁺. Compound 71C: LC-MS (ESI) m/z: 334 [M+H]⁺.

Compound 71 was purified with chiral HPLC to give Compound 71-1 and Compound 71-2. Compound 71-1: LC-MS (ESI) m/z: 531 [M+H]⁺; ¹H-NMR (DMSO-d₆, 400 MHz): δ (ppm) 0.65-1.08 (dd, J=6.0 Hz, 3H), 3.69 (s, 3H), 4.34-4.35 (m, 1H), 6.80-6.82 (m, 1H), 7.10-7.28 (m, 4H), 7.36-7.65 (m, 8H); Chiral separation condition: MeOH contained 0.2% Methanol Ammonia; IC (4.6*250 mm, 5 μm); retention time: 2.70 minutes (78.5%), 3.83 minutes (21.4%). Compound 71-2: LC-MS (ESI) m/z: 531 [M+H]⁺; ¹H-NMR (DMSO-d₆, 400 MHz): δ (ppm)) 0.65-1.08 (dd, J=6.0 Hz, 3H), 3.69 (s, 3H), 4.34-4.35 (m, 1H), 6.81-6.83 (m, 1H), 7.09-7.28 (m, 4H), 7.36-7.66 (m, 8H); Chiral separation condition: MeOH contained 0.2% Methanol Ammonia; IC (4.6*250 mm, 5 μm); retention time: 3.87 minutes (22.8%), 5.97 minutes (77.1%).

### Example 72

### Synthesis of 3-(5,7-bis(4-chlorophenyl)-8-hydroxy-6-oxo-5,7-diazaspiro[3.4]octan-8-yl)benzonitrile

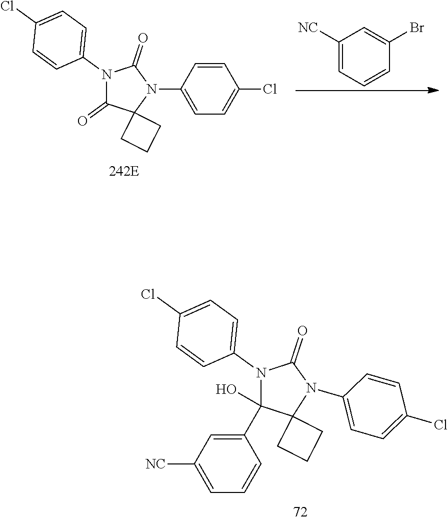 [SMILES: O=C1N(c2ccc(Cl)cc2)C(=O)C2(CCC2)N1c1ccc(Cl)cc1 ; N#Cc1cccc(Br)c1 ; N#Cc1cccc(C2(O)N(c3ccc(Cl)cc3)C(=O)N(c3ccc(Cl)cc3)C23CCC3)c1]

Compound 72 was synthesized by employing the procedure described for Compound 125 using Compounds 242E and 3-bromobenzonitrile in lieu of Compound 2C and 3-bromo-N,N-dimethylaniline. LC-MS (ESI) m/z: 464 [M+H]⁺; ¹H-NMR (400 MHz, CDCl₃): δ (ppm) 0.79-0.86 (m, 1H), 1.12-1.17 (m, 1H), 1.70-1.75 (m, 1H), 2.03-2.08 (m, 1H), 2.28-2.32 (m, 1H), 2.70-2.75 (m, 1H), 3.70 (s, 1H), 7.15-7.17 (m, 2H), 7.28-7.37 (m, 4H), 7.43-7.48 (m, 3H), 7.63-7.70 (m, 2H), 7.96-7.98 (m, 1H).

### Example 73

### Synthesis of 1,3-bis(4-bromophenyl)-4-(3-chlorophenyl)-4-hydroxy-5-methylimidazolidin-2-one, (5S)-1,3-bis(4-bromophenyl)-4-(3-chlorophenyl)-4-hydroxy-5-methylimidazolidin-2-one, and (5R)-1,3-bis(4-bromophenyl)-4-(3-chlorophenyl)-4-hydroxy-5-methylimidazolidin-2-one

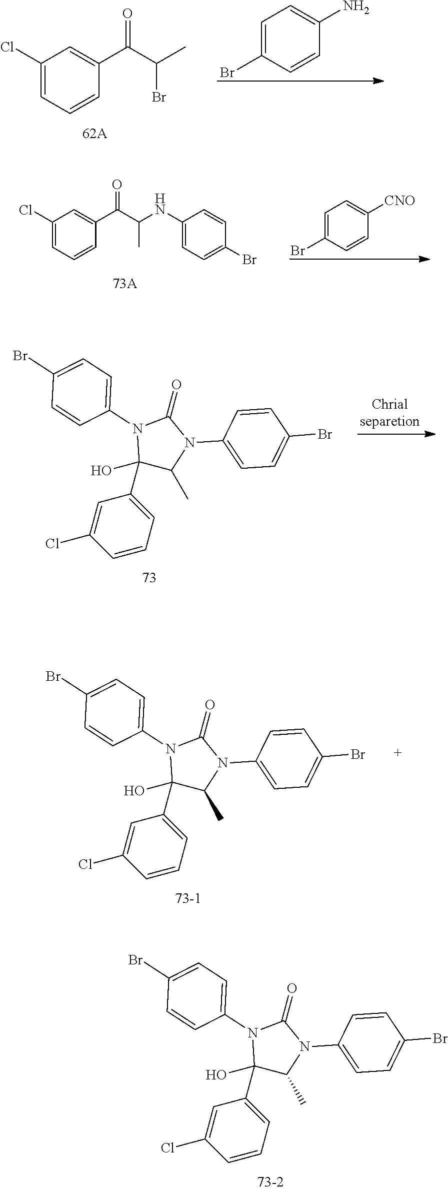

Compounds 73A and 73 were synthesized by employing the procedures described for Compounds 1B and 1 using Compounds 62A, heated at 60° C., and 73A in lieu of Compounds 1A, stirred at room temperature, and 1B. Compound 73A: LC-MS (ESI) m/z: 338 [M+H]⁺; ¹H-NMR (CDCl₃, 400 MHz): δ (ppm) 1.46 (d, J=7.2 Hz, 3H), 4.67 (d, J=8.0 Hz, 1H), 4.97-5.04 (m, 1H), 6.53 (d, J=8.8 Hz, 2H), 7.25 (d, J=8.8 Hz, 2H), 7.46 (t, J=8.0 Hz, 1H), 7.59 (q, J=7.2 Hz, 1H), 7.87 (d, J=8.0 Hz, 1H), 7.96 (s, 1H). Compound 73: LC-MS (ESI) m/z: 535 [M+H]⁺; ¹H-NMR (CDCl₃, 400 MHz): δ (ppm) 0.72-1.24 (dd, J=6.4 Hz, 3H), 3.78 (s, 1H), 4.20, 4.30 (q, J=6.4 Hz, 1H), 7.16-7.18 (m, 2H), 7.27-7.36 (m, 7H), 7.49-7.58 (m, 2H), 7.59 (s, 1H).

Compound 73 was separated with chiral HPLC to give Compound 73-1 and Compound 73-2. Compound 73-1: LC-MS (ESI) m/z: 535 [M+H]⁺; ¹H-NMR (DMSO-d₆, 400 MHz): δ (ppm) 0.65-1.07 (dd, J=6.4 Hz, 3H), 4.37, 4.51 (q, J=6.4 Hz, 1H), 7.31-7.63 (m, 12H); Chiral separation condition: MeOH contained 0.2% Methanol Ammonia; RegisCell (4.6*250 mm, 5 μm); retention time: 2.65 minutes (78%), 4.0 minutes (22%). Compound 73-2: LC-MS (ESI) m/z: 535 [M+H]⁺; ¹H-NMR (DMSO-d₆, 400 MHz): δ (ppm) 0.65-1.07 (d, J=6.4 Hz, 3H), 4.37, 4.51 (q, J=6.4 Hz, 1H), 7.33-7.63 (m, 12H); Chiral separation condition: MeOH contained 0.2% Methanol Ammonia; RegisCell (4.6*250 mm, 5 μm); retention time: 3.55 minutes (21%), 6.13 minutes (74%).

### Example 74

### Synthesis of 1,3-bis(4-bromophenyl)-4-hydroxy-5-methyl-4-(3-(trifluoromethyl)phenyl)imidazolidin-2-one, (5S)-1,3-bis(4-bromophenyl)-4-hydroxy-5-methyl-4-(3-(trifluoromethyl)phenyl)imidazolidin-2-one, and (5R)-1,3-bis(4-bromophenyl)-4-hydroxy-5-methyl-4-(3-(trifluoromethyl)phenyl)imidazolidin-2-one

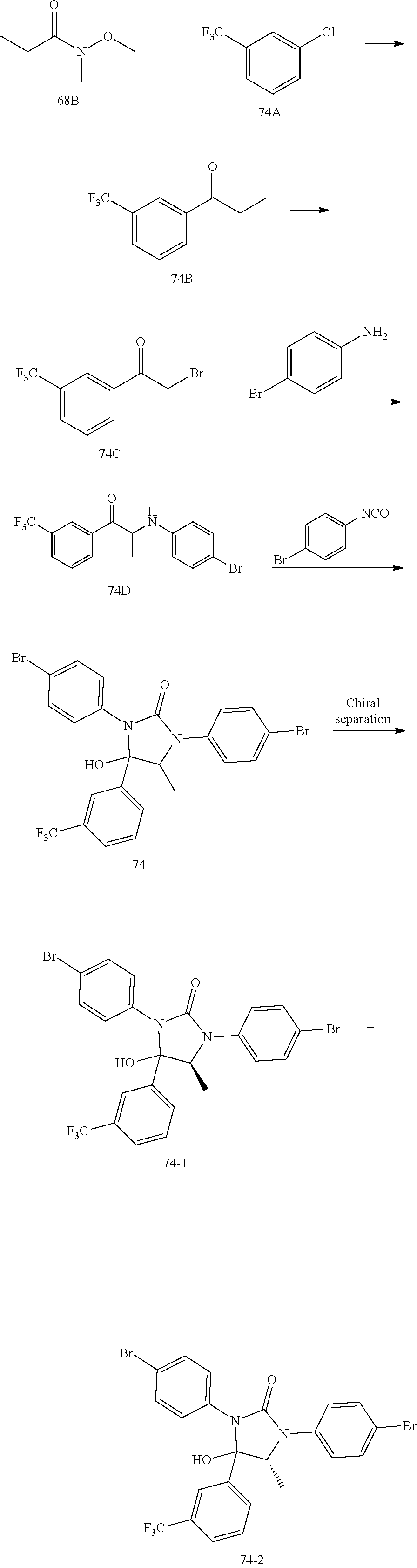

Compounds 74B, 74C, 74D, and 74 were synthesized by employing the procedures described for Compounds 59B, 13B, 1B, and 1 using Compounds 68B, 74A, 74B, 74C, heated at 60° C., and 74D in lieu of N-methoxy-N-methylacetamide, Compounds 59A, 13A, 1A, stirred at room temperature, and 1B. Compound 74B: LC-MS (ESI) m/z: 203 [M+H]⁺; ¹H-NMR (CDCl₃, 400 MHz): δ (ppm) 1.26 (t, J=7.2 Hz, 3H), 3.05 (q, J=7.2 Hz, 2H), 7.62 (t, J=7.6 Hz, 1H), 7.82 (d, J=7.6 Hz, 1H), 8.16 (d, J=7.6 Hz, 1H), 8.23 (s, 1H). Compound 74C: LC-MS (ESI) m/z: 281 [M+H]⁺; ¹H-NMR (CDCl₃, 400 MHz): δ (ppm) 1.94 (d, J=6.8 Hz, 3H), 5.25-5.30 (m, 1H), 7.65 (t, J=7.6 Hz, 1H), 7.86 (d, J=7.6 Hz, 1H), 8.22 (d, J=7.6 Hz, 1H), 8.29 (s, 1H). Compound 74D: LC-MS (ESI) m/z: 372 [M+H]⁺; ¹H-NMR (CDCl₃, 400 MHz): δ (ppm) 1.48 (d, J=6.8 Hz, 3H), 4.85-4.95 (m, 1H), 5.05-5.10 (m, 1H), 6.56 (d, J=8.0 Hz, 2H), 7.27 (d, J=8.0 Hz, 2H), 7.67 (t, J=8.0 Hz, 1H), 7.90 (d, J=8.0 Hz, 1H), 8.18 (d, J=8.0 Hz, 1H), 8.25 (s, 1H).

Compound 74 was separated with chiral HPLC to give Compound 74-1 and Compound 74-2. Compound 74-1: LC-MS (ESI) m/z: 569 [M+H]⁺; ¹H-NMR (CDCl₃, 400 MHz): δ (ppm) 1.26 (d, J=6.4 Hz, 3H), 4.16-4.21 (m, 1H), 7.16-7.24 (m, 6H), 7.37-7.48 (m, 3H), 7.52-7.57 (m, 1H), 7.60-7.62 (m, 1H), 7.83 (s, 1H). Chiral separation condition: MeOH contained 0.2% Methanol Ammonia; RegisCell (4.6*250 mm, 5 μm); retention time: 2.41 minutes (76%), 4.38 minutes (21%). Compound 74-2: LC-MS (ESI) m/z: 569 [M+H]⁺; ¹H-NMR (CDCl₃, 400 MHz): δ (ppm) 1.26 (d, J=6.4 Hz, 3H), 4.16-4.21 (m, 1H), 7.15-7.24 (m, 6H), 7.37-7.47 (m, 3H), 7.53-7.57 (m, 1H), 7.59-7.61 (m, 1H), 7.83 (s, 1H). Chiral separation condition: co-solvent: MeOH contained 0.2% Methanol Ammonia; RegisCell (4.6*250 mm, m); retention time: 3.84 minutes (21%), 6.95 minutes (78%).

### Example 75

### Synthesis of 1,3-bis(4-chlorophenyl)-5-ethyl-4-hydroxy-4-(m-tolyl)imidazolidin-2-one, (5S)-1,3-bis(4-chlorophenyl)-5-ethyl-4-hydroxy-4-(m-tolyl)imidazolidin-2-one, and (5R)-1,3-bis(4-chlorophenyl)-5-ethyl-4-hydroxy-4-(m-tolyl)imidazolidin-2-one

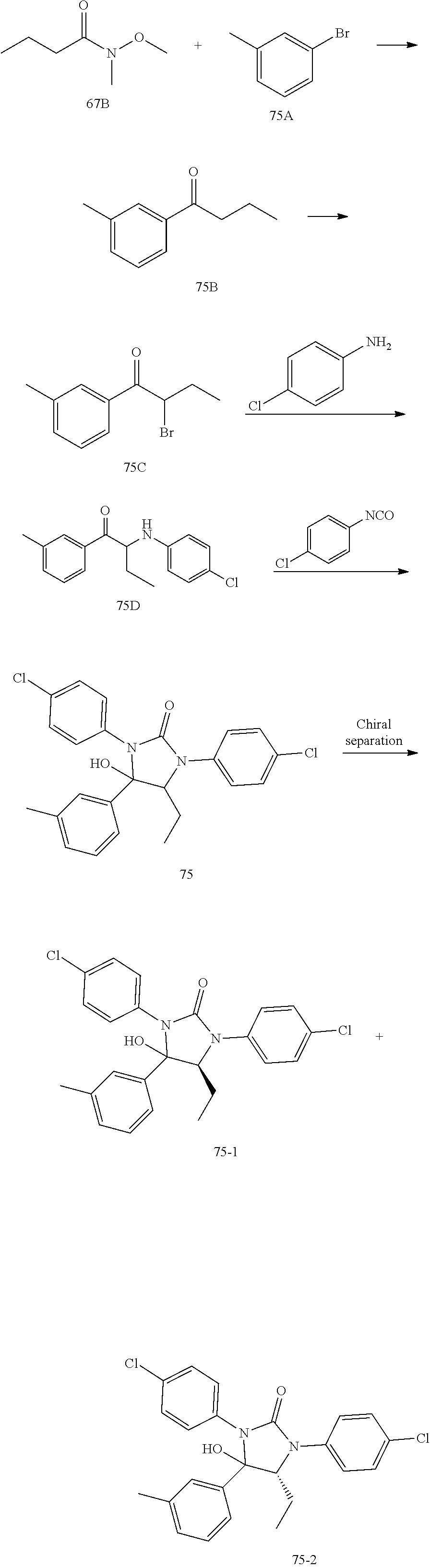

Compounds 75B, 75C, 75D, and 75 were synthesized by employing the procedures described for Compounds 59B, 13B, 1B, and 1 using Compounds 67B, 75A, 75B, 75C, 4-chloroaniline, heated at 60° C., 75D, and 1-chloro-4-isocyanatobenzene in lieu of N-methoxy-N-methylacetamide, Compounds 59A, 13A, 1A, 4-bromoaniline, stirred at room temperature, 1B, and 1-bromo-4-isocyanatobenzene. Compound 75B: LC-MS (ESI) m/z: non-ionizable compound under routine conditions used; ¹H-NMR (CDCl₃, 400 MHz): δ (ppm) 1.01 (t, J=7.2 Hz, 3H), 1.66-1.82 (m, 2H), 2.41 (s, 3H), 2.94 (t, J=7.2 Hz, 2H), 7.33-7.38 (m, 2H), 7.75-7.78 (m, 2H). Compound 75C: LC-MS (ESI) m/z: non-ionizable compound under routine conditions used; ¹H-NMR (CDCl₃, 400 MHz): δ (ppm) 1.08 (t, J=7.2 Hz, 3H), 2.10-2.75 (m, 2H), 2.43 (s, 3H), 5.08 (t, J=7.2 Hz, 1H), 7.35-7.42 (m, 2H), 7.80-7.82 (m, 2H). Compound 75D: LC-MS (ESI) m/z: 288 [M+H]⁺; ¹H-NMR (CDCl₃, 400 MHz): δ (ppm) 0.87 (t, J=7.2 Hz, 3H), 1.68-1.77 (m, 1H), 2.01-2.09 (m, 1H), 2.44 (s, 3H), 4.75 (d, J=8.0 Hz, 1H), 4.99-5.04 (m, 1H), 6.57 (d, J=8.8 Hz, 2H), 7.24 (d, J=8.8 Hz, 2H), 7.37-7.43 (m, 2H), 7.76-7.78 (m, 2H).

Compound 75 was separated with chiral HPLC to afford Compound 75-1 and Compound 75-2. Compound 75-1: LC-MS (ESI) m/z: 441 [M+H]⁺; ¹H-NMR (CDCl₃, 400 MHz): δ (ppm) 0.35-0.75 (dt, J=7.2 Hz, 3H), 1.80-1.86 (m, 2H), 2.33 (s, 3H), 3.40-3.50 (m, 1H), 4.17-4.22 (m, 1H), 7.08-7.25 (m, 5H), 7.27-7.49 (m, 7H); Chiral separation condition: MeOH contained 0.2% methanol ammonia; RegisCell (4.6*250 mm, 5 μm); retention time: 2.24 minutes (71.1%), 3.93 minutes (25.5%). Compound 75-2: LC-MS (ESI) m/z: 441 [M+H]⁺; ¹H-NMR (CDCl₃, 400 MHz): δ (ppm) 0.35-0.75 (dt, J=7.2 Hz, 3H), 1.80-1.86 (m, 2H), 2.33 (s, 3H), 3.40-3.50 (m, 1H), 4.17-4.22 (m, 1H), 7.08-7.25 (m, 5H), 7.27-7.49 (m, 7H); Chiral separation condition: MeOH contained 0.2% methanol ammonia; RegisCell (4.6*250 mm, 5 μm); retention time: 2.96 minutes (27.6%), 5.33 minutes (70.8%).

### Example 76

### Synthesis of 1,3-bis(4-bromophenyl)-5-ethyl-4-hydroxy-4-(m-tolyl)imidazolidin-2-one, (5S)-1,3-bis(4-bromophenyl)-5-ethyl-4-hydroxy-4-(m-tolyl)imidazolidin-2-one, and (5R)-1,3-bis(4-bromophenyl)-5-ethyl-4-hydroxy-4-(m-tolyl)imidazolidin-2-one

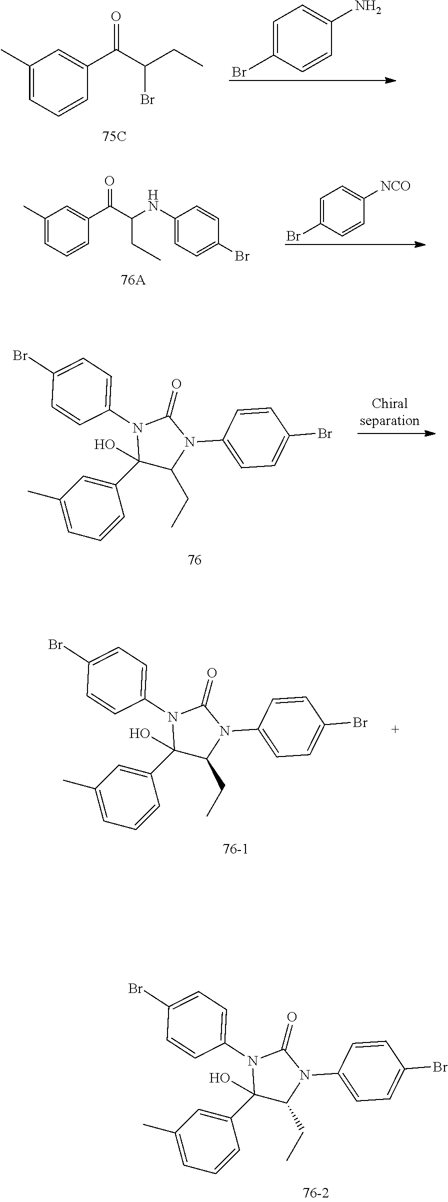

Compounds 76A and 76 were synthesized by employing the procedures described for Compounds 1B and 1 using Compounds 75C, heated at 60° C., and 76A in lieu of Compounds 1A, stirred at room temperature, and 1B. Compound 76A: LC-MS (ESI) m/z: 332 [M+H]⁺; ¹H-NMR (CDCl₃, 400 MHz): δ (ppm) 0.87 (t, J=7.2 Hz, 3H), 1.68-1.77 (m, 1H), 2.01-2.09 (m, 1H), 2.44 (s, 3H), 4.77 (d, J=8.0 Hz, 1H), 4.99-5.04 (m, 1H), 6.57 (d, J=8.8 Hz, 2H), 7.24 (d, J=8.8 Hz, 2H), 7.37-7.44 (m, 2H), 7.76-7.78 (m, 2H).

Compound 76 was separated with chiral HPLC to afford Compound 76-1 and Compound 76-2. Compound 76-1: LC-MS (ESI) m/z: 529 [M+H]⁺; ¹H-NMR (CDCl₃, 400 MHz): δ (ppm) 0.36-0.75 (dt, J=7.2 Hz, 3H), 1.81-1.86 (m, 2H), 2.33 (s, 3H), 3.31, 3.80 (s, 1H), 4.17-4.22 (m, 1H), 7.09-7.25 (m, 5H), 7.27-7.52 (m, 7H); Chiral separation condition: MeOH contained 0.2% methanol ammonia; RegisCell (4.6*250 mm, 5 μm); retention time: 2.37 minutes (74.9%), 4.21 minutes (24.5%). Compound 76-2: LC-MS (ESI) m/z: 529 [M+H]⁺; ¹H-NMR (CDCl₃, 400 MHz): δ (ppm) 0.36-0.75 (dt, J=7.2 Hz, 3H), 1.81-1.86 (m, 2H), 2.29 (s, 3H), 3.31, 3.80 (s, 1H), 4.17-4.22 (m, 1H), 7.02-7.25 (m, 5H), 7.27-7.46 (m, 7H); Chiral separation condition: MeOH contained 0.2% methanol ammonia; RegisCell (4.6*250 mm, 5 μm); retention time: 3.36 minutes (23.9%), 6.28 minutes (74.4%).

### Example 77

### Synthesis of 1,3-bis(4-chlorophenyl)-4-hydroxy-5-methyl-4-(3-(trifluoromethyl)phenyl)imidazolidin-2-one, (5S)-1,3-bis(4-chlorophenyl)-4-hydroxy-5-methyl-4-(3-(trifluoromethyl)phenyl)imidazolidin-2-one, and (5R)-1,3-bis(4-chlorophenyl)-4-hydroxy-5-methyl-4-(3-(trifluoromethyl)phenyl)imidazolidin-2-one

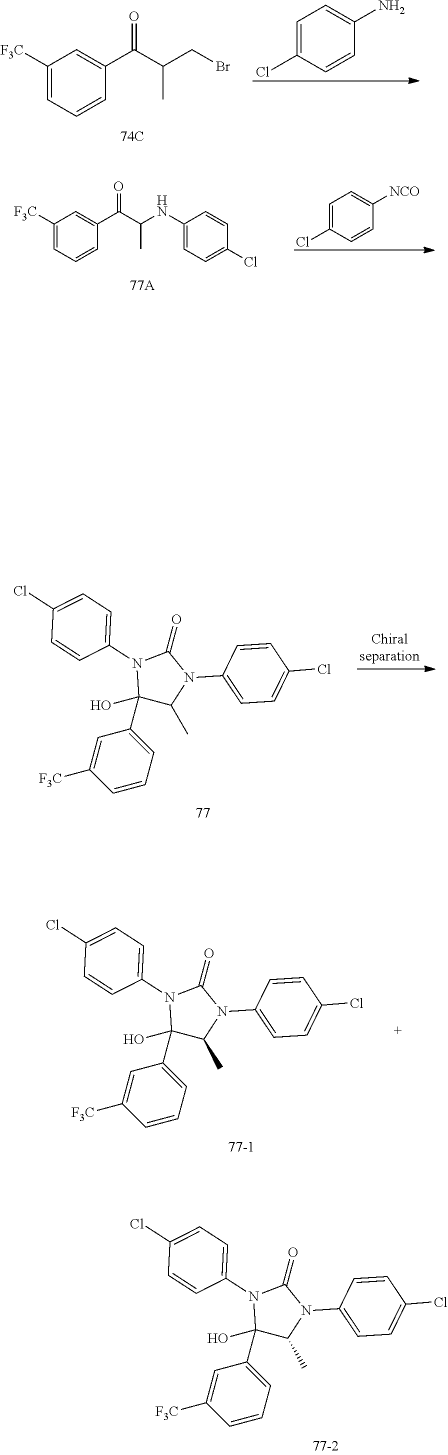

Compounds 77A and 77 were synthesized by employing the procedures described for Compounds 1B and 1 using Compounds 74C, 4-chloroaniline, heated at 60° C., 77A, and 1-chloro-4-isocyanatobenzene in lieu of Compounds 1A, 4-bromoaniline, stirred at room temperature, 1B, and 1-bromo-4-isocyanatobenzene. Compound 77A: LC-MS (ESI) m/z: 328 [M+H]⁺.

Compound 77 was separated with chiral HPLC to give Compound 77-1 and Compound 77-2. Compound 77-1: LC-MS (ESI) m/z: 481 [M+H]⁺; ¹H-NMR (CDCl₃, 400 MHz): δ (ppm) 1.27 (d, J=6.0 Hz, 3H), 4.14 (s, 1H), 4.19-4.24 (m, 1H), 7.10 (d, J=8.8 Hz, 2H), 7.23-7.27 (m, 4H), 7.35 (d, J=8.8 Hz, 2H), 7.44 (t, J=8.0 Hz, 1H), 7.57 (d, J=8.0 Hz, 1H), 7.63 (d, J=8.0 Hz, 1H), 7.85 (s, 1H); Chiral separation condition: MeOH contained 0.2% Methanol Ammonia; RegisCell (4.6*250 mm, 5 μm); retention time: 2.05 minutes (99%), 3.28 minutes (1%). Compound 77-2: LC-MS (ESI) m/z: 481 [M+H]⁺; ¹H-NMR (CDCl₃, 400 MHz): δ (ppm) 1.27 (d, J=6.4 Hz, 3H), 4.18-4.23 (m, 1H), 7.10 (d, J=7.6 Hz, 2H), 7.23-7.27 (m, 4H), 7.34 (d, J=8.0 Hz, 2H), 7.44 (t, J=8.0 Hz, 1H), 7.56 (d, J=8.0 Hz, 1H), 7.63 (d, J=8.0 Hz, 1H), 7.84 (s, 1H). Chiral separation condition: MeOH contained 0.2% Methanol Ammonia; RegisCell (4.6*250 mm, 5 m); retention time: 2.93 minutes (12%), 4.54 minutes (87%).

### Example 78

### Synthesis of 1,3-bis(4-chlorophenyl)-4-(3-cyclopropylphenyl)-4-hydroxyimidazolidin-2-one

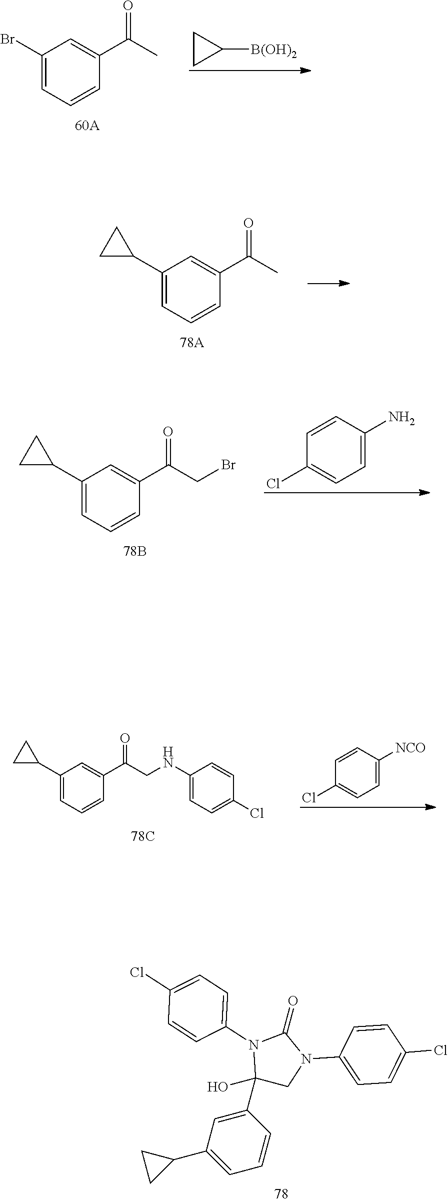

To a solution of Compound 60A (7.96 g, 40 mmol) in toluene (100 mL) was added cyclopropylboronic acid (6.88 g, 80 mmol), PdCl₂(dppf) (326 mg, 0.4 mmol), K₃PO₄ (25.4 g, 120 mmol), and H₂O (5 mL) and heated at 100° C. under nitrogen atmosphere for 3 hours. The reaction mixture was concentrated under reduced pressure. The residue was purified by column chromatography on silica gel (100% petroleum ether) to afford Compound 78A: LC-MS (ESI) m/z: non-ionizable compound under routine conditions used; ¹H-NMR (CDCl₃, 400 MHz): δ (ppm) 0.72-0.76 (m, 2H), 0.98-1.03 (m, 2H), 1.92-1.98 (m, 1H), 2.59 (s, 3H), 7.25-7.35 (m, 2H), 7.66-7.73 (m, 2H).

Compounds 78B, 78C, and 78 were synthesized by employing the procedures described for Compounds 13B, 1B, and 1 using Compounds 60A, using 1,4-dioxane as solvent, 78B, 4-chloroaniline, heated at 50° C., 78C, and 1-chloro-4-isocyanatobenzene in lieu of Compounds 13A, using diethyl ether as solvent, 1A, 4-bromoaniline, stirred at room temperature, 1B, and 1-bromo-4-isocyanatobenzene. Compound 78B: LC-MS (ESI) m/z: non-ionizable compound under routine conditions used; ¹H-NMR (CDCl₃, 400 MHz): δ (ppm) 0.72-0.77 (m, 2H), 1.00-1.05 (m, 2H), 1.93-1.95 (m, 1H), 4.44 (s, 2H), 7.29-7.38 (m, 2H), 7.69-7.77 (m, 2H). Compound 78C: LC-MS (ESI) m/z: 286 [M+H]⁺. Compound 78: LC-MS (ESI) m/z: 439 [M+H]⁺; ¹H-NMR (DMSO-d₆, 400 MHz): δ (ppm) 0.49-0.53 (m, 1H), 0.62-0.66 (m, 1H), 0.88-0.90 (m, 2H), 1.82-1.88 (m, 1H), 4.01 (d, J=10.8 Hz, 1H), 4.11 (d, J=10.8 Hz, 1H), 6.92-6.94 (m, 1H), 7.15-7.18 (m, 1H), 7.24-7.28 (m, 3H), 7.31-7.33 (m, 1H), 7.37-7.43 (m, 4H), 7.61 (s, 1H), 7.68-7.72 (m, 2H).

### Example 79

### Synthesis of 1,3-bis(4-bromophenyl)-4-(3-ethoxyphenyl)-4-hydroxyimidazolidin-2-one

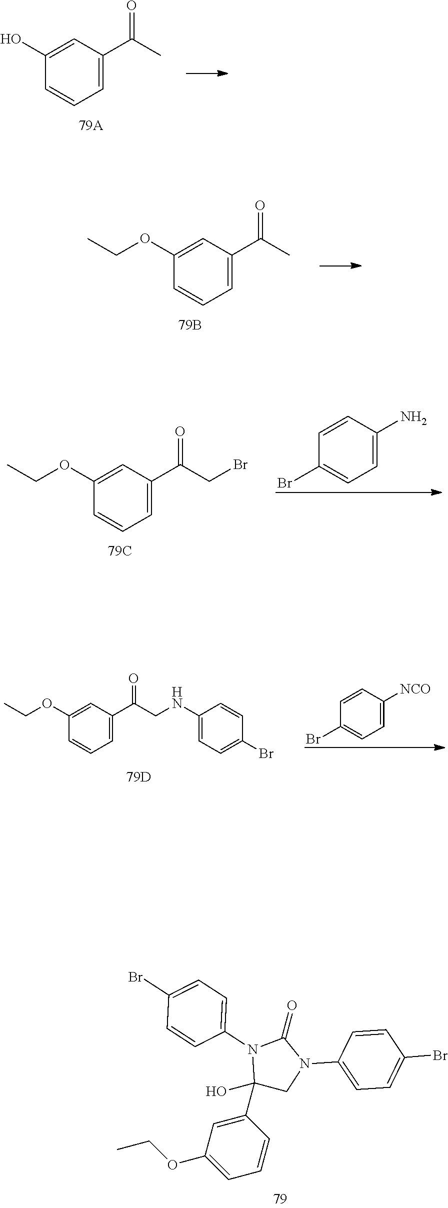

A mixture of Compound 79A (1.36 g, 10 mmol), potassium carbonate (2.89 g, 20 mmol), and iodoethane (2.86 g, 18.3 mmol) in acetone (30 mL) was stirred at 25° C. for 24 hours. The mixture was concentrated. The residue was diluted with water (100 mL) and extracted with dichloromethane (100 mL×2). The combined organic layers was washed with water (100 mL×2) and brine (100 mL), dried over anhydrous sodium sulfate, filtered, and concentrated to give a crude product, which was purified with flash column chromatography on silica gel (petroleum ether, 100% v/v) to yield Compound 79B: LC-MS (ESI) m/z: 165 [M+H]⁺.

Compounds 79C, 79D, and 79 were synthesized by employing the procedures described for Compounds 13B, 1B, and 1 using Compounds 79B, 79C, heated at 60° C., and 79D in lieu of Compounds 13A, 1A, stirred at room temperature, and 1B. Compound 79C: LC-MS (ESI) m/z: 243 [M+H]⁺. Compound 79D: LC-MS (ESI) m/z: 334 [M+H]⁺. Compound 79: LC-MS (ESI) m/z: 531 [M+H]⁺; ¹H-NMR (CDCl₃, 400 MHz): δ (ppm) 1.31 (t, J=7.2 Hz, 3H), 3.97-4.03 (m, 2H), 4.14 (d, J=10.4 Hz, 1H), 4.24 (d, J=10.4 Hz, 1H), 6.70 (s, 1H), 6.81-6.84 (m, 1H), 7.24-7.25 (m, 3H), 7.36-7.39 (m, 2H), 7.47-7.54 (m, 4H), 7.66-7.69 (m, 2H).

### Example 80

### Synthesis of 1,3-bis(4-chlorophenyl)-5-ethyl-4-hydroxy-4-(3-(trifluoromethyl)phenyl)imidazolidin-2-one, (5S)-1,3-bis(4-chlorophenyl)-5-ethyl-4-hydroxy-4-(3-(trifluoromethyl)phenyl)imidazolidin-2-one, and (5R)-1,3-bis(4-chlorophenyl)-5-ethyl-4-hydroxy-4-(3-(trifluoromethyl)phenyl)imidazolidin-2-one

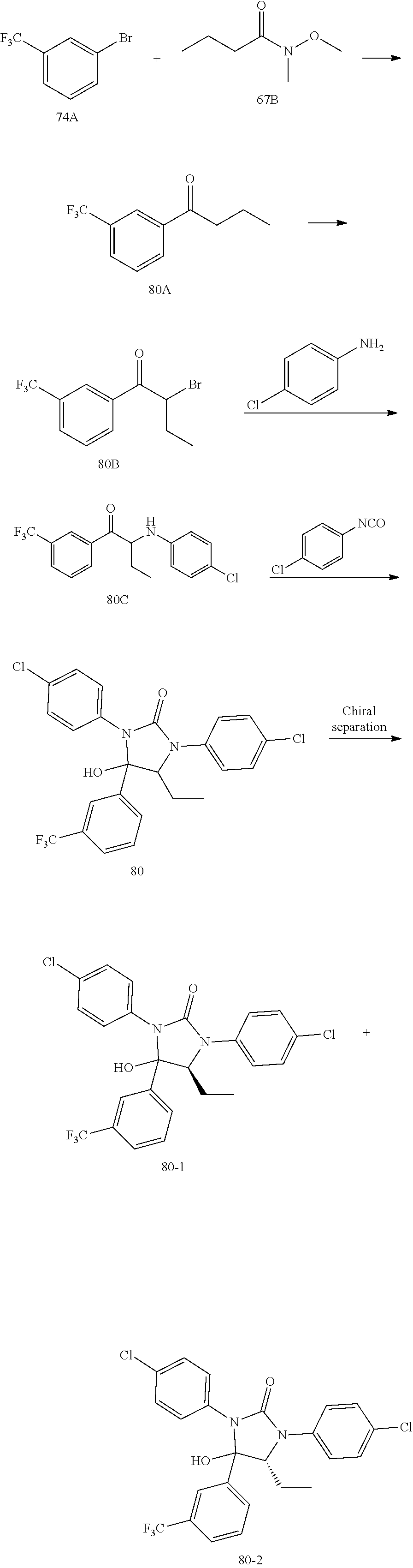

Compounds 80A, 80B, 80C, and 80 were synthesized by employing the procedures described for Compounds 59B, 13B, 1B, and 1 using Compounds 67B, 74A, 80A, 80B, 4-chloroaniline, heated at 60° C., 80C, and 1-chloro-4-isocyanatobenzene in lieu of N-methoxy-N-methylacetamide, Compounds 59A, 13A, 1A, 4-bromoaniline, stirred at room temperature, 1B, and 1-bromo-4-isocyanatobenzene. Compound 80B: LC-MS (ESI) m/z: non-ionizable compound under routine conditions used; ¹H-NMR (CDCl₃, 400 MHz): δ (ppm) 1.02 (t, J=7.6 Hz, 3H), 1.75-1.84 (m, 2H), 2.98 (t, J=7.2 Hz, 2H), 7.61 (t, J=8.0 Hz, 1H), 7.81 (d, J=7.6 Hz, 1H), 8.14 (d, J=7.6 Hz, 1H), 8.21 (s, 1H). Compound 80C: LC-MS (ESI) m/z: non-ionizable compound under routine conditions used; ¹H-NMR (CDCl₃, 400 MHz): δ (ppm) 1.11 (t, J=7.2 Hz, 3H), 2.13-2.28 (m, 2H), 5.03 (t, J=6.8 Hz, 1H), 7.64 (t, J=8.0 Hz, 1H), 7.85 (d, J=7.6 Hz, 1H), 8.20 (d, J=8.0 Hz, 1H), 8.27 (s, 1H). Compound 80D: LC-MS (ESI) m/z: 342 [M+H]⁺; ¹H-NMR (CDCl₃, 400 MHz): δ (ppm) 0.90 (t, J=7.6 Hz, 3H), 1.68-1.76 (m, 1H), 2.03-2.12 (m, 1H), 4.67 (d, J=8.0 Hz, 1H), 4.99-5.03 (m, 1H), 6.59-6.64 (m, 2H), 7.11-7.15 (m, 2H), 7.66 (t, J=7.6 Hz, 1H), 7.87 (d, J=7.6 Hz, 1H), 8.16 (d, J=8.0 Hz, 1H), 8.23 (s, 1H).

Compound 80 was separated with chiral HPLC to give Compound 80-1 and Compound 80-2. Compound 80-1: LC-MS (ESI) m/z: 495 [M+H]⁺; ¹H-NMR (CDCl₃, 400 MHz): δ (ppm) 0.27-0.69 (dt, J=7.6 Hz, 3H), 1.26-1.88 (m, 2H), 4.08-4.21 (m, 2H), 7.06-7.20 (m, 3H), 7.24-7.45 (m, 6H), 7.53-7.56 (m, 1H), 7.66 (d, J=8.4 Hz, 1H), 7.82-7.86 (m, 1H); Chiral separation condition: MeOH contained 0.2% methanol ammonia; RegisCell (4.6*250 mm, 5 μm); retention time: 2.11 minutes (78.2%), 3.63 minutes (21.8%). Compound 80-2: LC-MS (ESI) m/z: 495 [M+H]⁺; ¹H-NMR (CDCl₃, 400 MHz): δ (ppm) 0.30-0.70 (dt, J=7.6 Hz, 3H), 1.26-1.86 (m, 2H), 3.96 (s, 1H), 4.14-4.17 (m, 1H), 7.09-7.15 (m, 2H), 7.22-7.46 (m, 7H), 7.54-7.56 (m, 1H), 7.69 (d, J=8.0 Hz, 1H), 7.83-7.87 (m, 1H); Chiral separation condition: MeOH contained 0.2% methanol ammonia; RegisCell (4.6*250 mm, 5 μm); retention time: 3.05 minutes (23.7%), 4.65 minutes (74.3%).

### Example 81

### Synthesis of 4-(3-chlorophenyl)-1,3-bis(4-chlorophenyl)-5-ethyl-4-hydroxyimidazolidin-2-one, (5S)-4-(3-chlorophenyl)-1,3-bis(4-chlorophenyl)-5-ethyl-4-hydroxyimidazolidin-2-one, and (5R)-4-(3-chlorophenyl)-1,3-bis(4-chlorophenyl)-5-ethyl-4-hydroxyimidazolidin-2-one

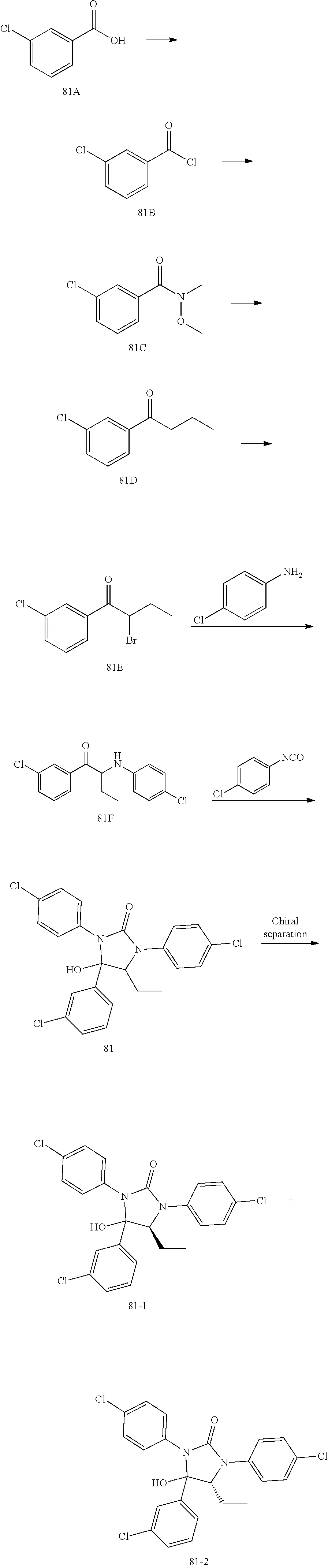

The mixture of Compound 81A (3 g, 19 mmol) and thionyl chloride (10 mL) was stirred at reflux overnight. The reaction mixture was concentrated under reduced pressure to yield a crude Compound 81B was used directly in the next step without any purification.

Compounds 81C, 81D, 81E, 81F, and 81 were synthesized by employing the procedures described for Compounds 42B, 42C, 13B, 1B, and 1 using Compounds 81B, 81C, propylmagnesium bromide, stirred at −78° C., 81D, 81E, 4-chloroaniline, heated at 60° C., 81F, and 1-chloro-4-isocyanatobenzene in lieu of Compounds 42A, 42B, ethylmagnesium bromide, stirred at −20° C., 13A, 1A, 4-bromoaniline, stirred at room temperature, 1B, and 1-bromo-4-isocyanatobenzene. Compound 81C: LC-MS (ESI) m/z: 200 [M+H]⁺. Compound 81D. ¹H-NMR (CDCl₃, 400 MHz): δ (ppm) 1.01 (t, J=7.2 Hz, 3H), 1.47-1.80 (m, 2H), 2.92 (t, J=7.2 Hz, 2H), 7.38-7.51 (m, 2H), 7.82-7.93 (m, 2H). Compound 81E. ¹H-NMR (CDCl₃, 400 MHz): δ (ppm) 1.08 (t, J=7.2 Hz, 3H), 2.11-2.25 (m, 2H), 5.01 (t, J=7.2 Hz, 1H), 7.40-7.56 (m, 2H), 7.86-7.98 (m, 2H). Compound 81F: LC-MS (ESI) m/z: 308 [M+H]⁺; ¹H-NMR (CDCl₃, 400 MHz): δ (ppm) 0.87 (t, J=7.2 Hz, 3H), 1.68-1.74 (m, 1H), 2.01-2.09 (m, 1H), 4.65-4.67 (m, 1H), 4.99-5.04 (m, 1H), 6.60 (d, J=8.8 Hz, 2H), 7.13 (d, J=8.8 Hz, 2H), 7.44-7.60 (m, 2H), 7.84-7.95 (m, 2H). Compound 81: LC-MS (ESI) m/z: 461 [M+H]⁺; ¹H-NMR (DMSO-d₆, 400 MHz): δ (ppm) 0.75 (t, J=7.2 Hz, 3H), 1.80-2.01 (m, 2H), 4.49-4.51 (m, 1H), 6.57 (s, 1H), 7.20-7.84 (m, 12H).

Compound 81 was separated with chiral HPLC to give Compound 81-1 and Compound 81-2. Compound 81-1: LC-MS (ESI) m/z: 461 [M+H]⁺; ¹H-NMR (DMSO-d₆, 400 MHz): δ (ppm) 0.58 (t, J=7.2 Hz, 3H), 1.67-1.81 (m, 2H), 4.34-4.35 (m, 1H), 6.45 (s, 1H), 7.08-7.85 (m, 12H); Chiral separation condition: MeOH contained 0.2% methanol ammonia; RegisCell (4.6*250 mm, 5 μm); retention time: 2.72 minutes (80%), 4.51 minutes (20%). Compound 81-2: LC-MS (ESI) m/z: 461 [M+H]⁺; ¹H-NMR (DMSO-d₆, 400 MHz): δ (ppm) 0.60 (t, J=7.2 Hz, 3H), 1.70-1.93 (m, 2H), 4.34-4.35 (m, 1H), 6.45 (s, 1H), 7.08-7.85 (m, 12H); Chiral separation condition: MeOH contained 0.2% methanol ammonia; RegisCell (4.6*250 mm, 5 μm); retention time: 3.68 minutes (20%), 5.89 minutes (80%).

### Example 82

### Synthesis of 1,3-bis(4-chlorophenyl)-5-ethyl-4-hydroxy-4-(3-(trifluoromethoxy)phenyl)imidazolidin-2-one, (5S)-1,3-bis(4-chlorophenyl)-5-ethyl-4-hydroxy-4-(3-(trifluoromethoxy)phenyl)imidazolidin-2-one, and (5R)-1,3-bis(4-chlorophenyl)-5-ethyl-4-hydroxy-4-(3-(trifluoromethoxy)phenyl)imidazolidin-2-one

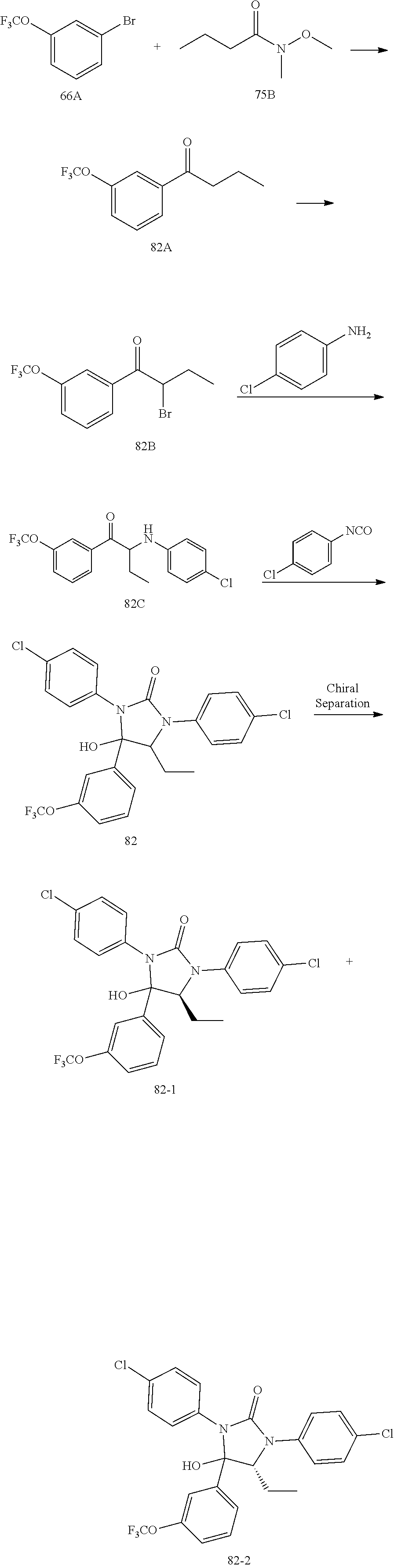

Compounds 82A, 82B, 82C, and 82 were synthesized by employing the procedures described for Compounds 59B, 13B, 1B, and 1 using Compounds 75B, 66A, 82A, 82B, 4-chloroaniline, heated at 60° C., 82C, and 1-chloro-4-isocyanatobenzene in lieu of N-methoxy-N-methylacetamide, Compounds 59A, 13A, 1A, 4-bromoaniline, stirred at room temperature, 1B, and 1-bromo-4-isocyanatobenzene. Compound 82A. ¹H-NMR (CDCl₃, 400 MHz): δ (ppm) 1.01 (t, J=8.0 Hz, 3H), 1.73-1.80 (m, 2H), 2.94 (t, J=8.0 Hz, 2H), 7.39-7.42 (m, 1H), 7.48-7.52 (m, 1H), 7.80 (s, 1H), 7.87-7.90 (m, 1H). Compound 82B. ¹H-NMR (CDCl₃, 400 MHz): δ (ppm) 1.10 (t, J=7.6 Hz, 3H), 2.11-2.27 (m, 2H), 4.79-5.01 (m, 1H), 7.43-7.46 (m, 1H), 7.52-7.56 (m, 1H), 7.86 (s, 1H), 7.93-7.96 (m, 1H). Compound 82C: LC-MS (ESI) m/z: 358 [M+H]⁺. Compound 82: LC-MS (ESI) m/z: 511 [M+H]⁺; ¹H-NMR (DMSO-d₆, 400 MHz): δ (ppm) 0.02, 0.35 (t, J=7.2 Hz, 3H), 0.90-1.11 (m, 2H), 4.06-4.13 (m, 1H), 6.98-7.10 (m, 4H), 7.17-7.26 (m, 6H), 7.37-7.45 (m, 2H), 7.69 (s, 1H).

Compound 82 was separated with chiral HPLC to give Compound 82-1 and Compound 82-2. Compound 82-1: LC-MS (ESI) m/z: 511 [M+H]⁺; ¹H-NMR (Acetone-d₆, 500 MHz): δ (ppm)) 0.40, 0.74 (t, J=7.5 Hz, 3H), 1.86-2.06 (m, 2H), 4.51-4.53 (m, 1H), 6.65 (s, 1H), 7.19-7.26 (m, 3H), 7.43-7.51 (m, 5H), 7.58-7.81 (m, 4H); Chiral separation condition: MeOH contained 0.2% Methanol Ammonia; OD-H (4.6*250 mm, 5 μm); retention time: 1.61 minutes (79%), 3.77 minutes (21%). Compound 82-2: LC-MS (ESI) m/z: 511 [M+H]⁺; ¹H-NMR (Acetone-d₆, 500 MHz): δ (ppm)) 0.25, 0.59 (t, J=7.5 Hz, 3H), 1.71-1.84 (m, 2H), 4.35-4.38 (m, 1H), 6.52 (s, 1H), 7.09-7.11 (m, 3H), 7.27-7.35 (m, 6H), 7.42-7.66 (m, 3H). Chiral separation condition: MeOH contained 0.2% Methanol Ammonia; OD-H (4.6*250 mm, 5 μm); retention time: 2.72 minutes (22%), 4.83 minutes (77%).

### Example 83

### Synthesis of 1,3-bis(4-bromophenyl)-5-ethyl-4-hydroxy-4-(3-(trifluoromethoxy)phenyl)imidazolidin-2-one, (5S)-1,3-bis(4-bromophenyl)-5-ethyl-4-hydroxy-4-(3-(trifluoromethoxy)phenyl)imidazolidin-2-one, and (5R)-1,3-bis(4-bromophenyl)-5-ethyl-4-hydroxy-4-(3-(trifluoromethoxy)phenyl)imidazolidin-2-one

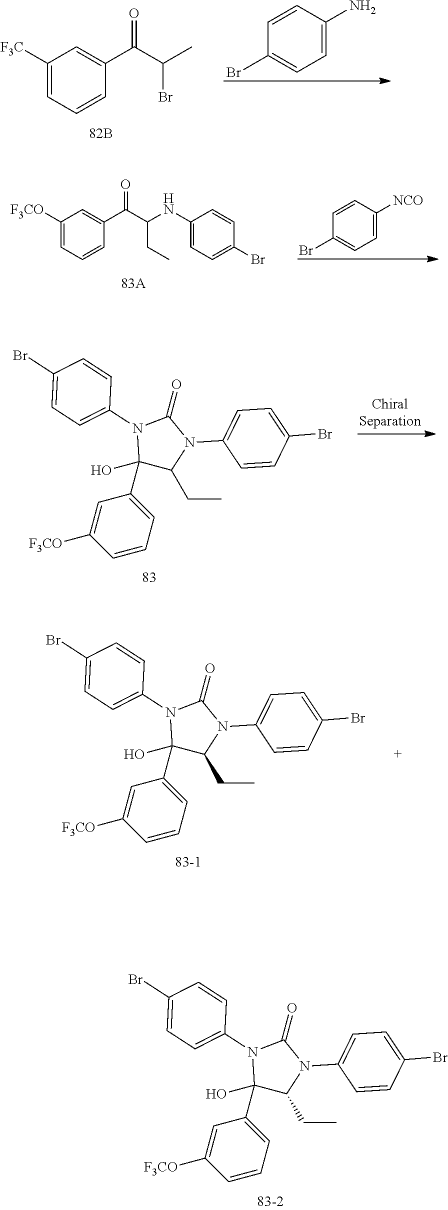

Compounds 83A and 83 were synthesized by employing the procedures described for Compounds 1B and 1 using Compound 82B, heated at 60° C., and 83A in lieu of Compounds 1A, stirred at room temperature, and 1B. Compound 83A: LC-MS (ESI) m/z: 402 [M+H]⁺. Compound 83: LC-MS (ESI) m/z: 599 [M+H]⁺; ¹H-NMR (DMSO-d₆, 400 MHz): δ (ppm) 0.25, 0.57 (t, J=7.2 Hz, 3H), 1.61-1.76 (m, 2H), 4.30-4.36 (m, 1H), 7.21-7.30 (m, 2H), 7.34-7.47 (m, 5H), 7.57-7.71 (m, 5H), 7.94 (s, 1H).

Compound 83 was separated with chiral HPLC to give Compound 83-1 and Compound 83-2. Compound 83-1: LC-MS (ESI) m/z: 599 [M+H]⁺; ¹H-NMR (Acetone-d₆, 500 MHz): δ (ppm)) 0.40, 0.74 (t, J=7.5 Hz, 3H), 1.87-1.98 (m, 2H), 4.50-4.53 (m, 1H), 6.65 (s, 1H), 7.24-7.43 (m, 6H), 7.48-7.59 (m, 4H), 7.66-7.80 (m, 2H). Chiral separation condition: MeOH contained 0.2% Methanol Ammonia; OZ-H (250*4.6 mm, 5 μm); retention time: 2.09 minutes (79%), 3.29 minutes (19%). Compound 83-2: LC-MS (ESI) m/z: 599 [M+H]⁺; ¹H-NMR (Acetone-d₆, 500 MHz): δ (ppm)) 0.24, 0.70 (t, J=7.5 Hz, 3H), 1.81-2.00 (m, 2H), 4.44-4.47 (m, 1H), 6.63 (s, 1H), 7.18-7.21 (m, 2H), 7.27-7.36 (m, 5H), 7.42-7.62 (m, 5H). Chiral separation condition: MeOH contained 0.2% Methanol Ammonia; OZ-H (250*4.6 mm, 5 μm); retention time: 2.89 minutes (19%), 4.23 minutes (81%).

### Example 84

### Synthesis of methyl 1,3-bis(4-bromophenyl)-4-hydroxy-5-methyl-2-oxoimidazolidine-4-carboxylate, methyl (5S)-1,3-bis(4-bromophenyl)-4-hydroxy-5-methyl-2-oxoimidazolidine-4-carboxylate, and methyl (5R)-1,3-bis(4-bromophenyl)-4-hydroxy-5-methyl-2-oxoimidazolidine-4-carboxylate

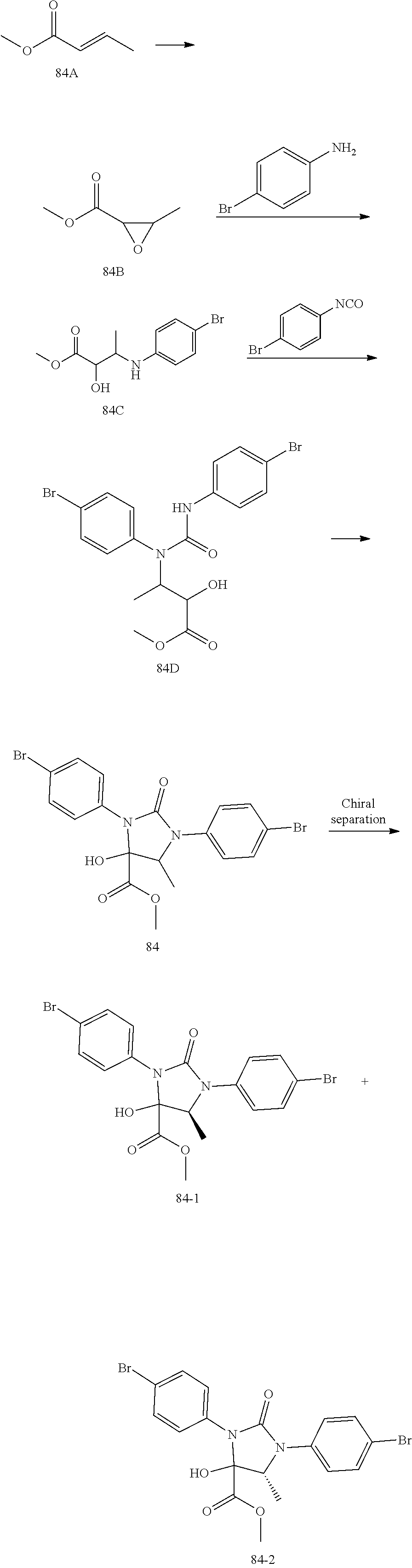

To a solution of Compound 84A (10 mmol) in dichloromethane (50 mL) was added m-CPBA (1.72 g, 10 mmol). The mixture was stirred at room temperature overnight and concentrated under reduced pressure. The crude product was purified with flash column chromatography on silica gel (ethyl acetate in petroleum, 20% v/v) to furnish Compound 84B. ¹H-NMR (CDCl₃, 400 MHz): δ (ppm) 1.40 (d, J=4.8 Hz, 3H), 3.20 (d, J=2.0 Hz, 1H), 3.23-3.25 (m, 1H), 3.78 (s, 3H).

A mixture of 4-bromoaniline (3.4 g, 20 mmol) and Compound 84B (2.9 g, 25 mmol) was heated at 145° C. for 2 hours. After cooled down to room temperature, the reaction mixture was concentrated and the residue was purified with flash column chromatography on silica gel (ethyl acetate in petroleum ether, from 0% to 5% v/v) to afford Compound 84C. LC-MS (ESI) m/z: 288 [M+H]⁺.

Compounds 84D and 84 were synthesized by employing the procedures described for Compounds 1 and 14 using Compounds 84C and 84D in lieu of Compounds 1B and 14D. Compound 84D. LC-MS (ESI) m/z: 485 [M+H]⁺.

Compound 84 was separated with chiral HPLC to furnish Compound 84-1 and Compound 84-2. Compound 84-1: LC-MS (ESI) m/z: 483 [M+H]⁺; ¹H-NMR (CDCl₃, 400 MHz): δ (ppm) 1.26 (d, J=6.2 Hz, 3H), 3.75 (s, 3H), 4.28 (s, 1H), 4.47 (q, J=6.2 Hz, 1H), 7.18-7.25 (m, 4H), 7.40-7.45 (m, 4H); Chiral separation condition: MeOH contained 0.2% methanol ammonia; RegisCell (4.6*250 mm, 5 μm); retention time: 2.65 minutes.

Compound 84-2: LC-MS (ESI) m/z: 483 [M+H]⁺; ¹H-NMR (CDCl₃, 400 MHz): δ (ppm) 1.26 (d, J=6.2 Hz, 3H), 3.75 (s, 3H), 4.47 (q, J=6.2 Hz, 1H), 7.18-7.25 (m, 4H), 7.40-7.45 (m, 4H); Chiral separation condition: MeOH contained 0.2% methanol ammonia; RegisCell (4.6*250 mm, 5 μm); retention time: 4.11 minutes.

### Example 85

### Synthesis of 1,3-bis(4-chlorophenyl)-4-(3,5-dichlorophenyl)-4-hydroxyimidazolidin-2-one

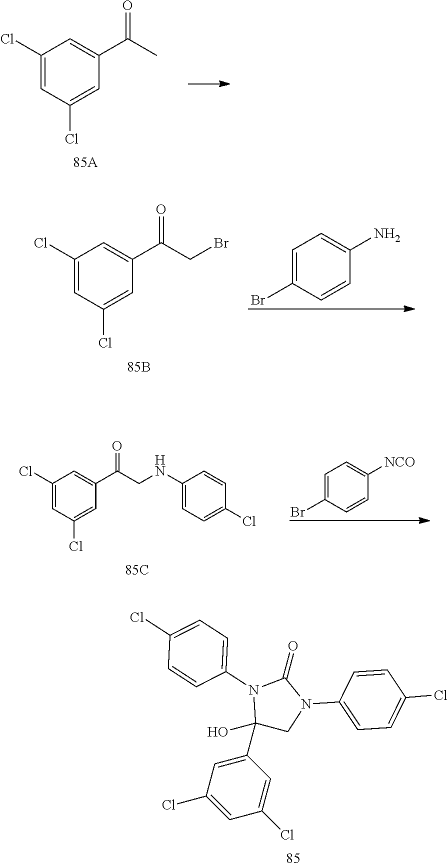

Compounds 85B, 85C, and 85 were synthesized by employing the procedures described for Compounds 13B, 1B, and 1 using Compounds 85A, 85B, and 85C in lieu of Compounds 13A, 1A, and 1B. Compound 85B: LC-MS (ESI) m/z: 267 [M+H]⁺. Compound 85C: LC-MS (ESI) m/z: 314 [M+H]⁺; ¹H-NMR (CDCl₃, 400 MHz): δ (ppm) 4.53 (d, J=4.4 Hz, 2H), 4.84 (s, 1H), 6.62-6.64 (m, 2H), 7.17 (dd, J=6.8, 2.0 Hz, 2H), 7.62 (t, J=2.0 Hz, 1H), 7.86 (d, J=1.6 Hz, 2H). Compound 85: LC-MS (ESI) m/z: 467 [M+H]⁺; ¹H-NMR (DMSO-d₆, 400 MHz): δ (ppm) 4.02 (d, J=10.8 Hz, 1H), 4.19 (d, J=10.8 Hz, 1H), 7.31-7.44 (m, 6H), 7.53 (d, J=1.6 Hz, 1H), 7.66-7.70 (m, 4H), 7.98 (s, 1H).

### Example 86

### Synthesis of 1,3-bis(4-bromophenyl)-4-hydroxy-4,5-diphenylimidazolidin-2-one, (5S)-1,3-bis(4-bromophenyl)-4-hydroxy-4,5-diphenylimidazolidin-2-one, and (5R)-1,3-bis(4-bromophenyl)-4-hydroxy-4,5-diphenylimidazolidin-2-one

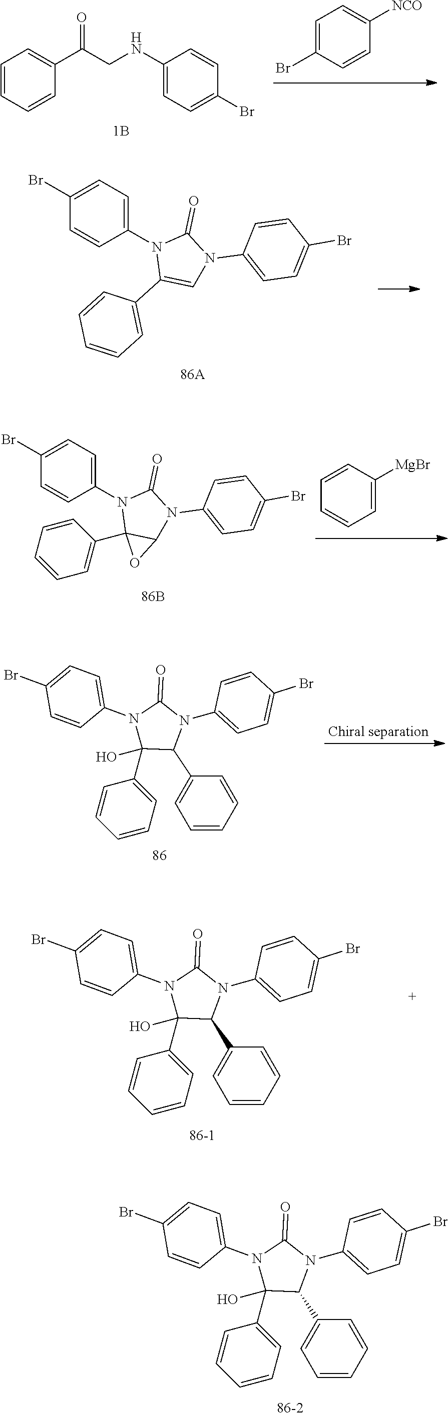

To a solution of Compound 1B (500 mg, 1.73 mmol) in dichloromethane (20 mL) was added 1-bromo-4-isocyanatobenzene (375 mg, 1.9 mmol) and stirred at 25° C. for 16 hours. To the mixture was added TFA (0.2 mL) and stirred at 25° C. for another 16 hours. The reaction mixture was diluted with dichloromethane (30 mL), washed with aqueous HCl solution (1 N, 20 mL), water (20 mL) and brine (20 mL), and concentrated to give a residue. The residue was diluted with a mixed solvent of petroleum ether (50 mL) and ethyl acetate (20 mL), filtered, and dried to afford Compound 86A: LC-MS (ESI) m/z: 469 [M+H]⁺; ¹H-NMR (DMSO-d₆, 400 MHz): δ (ppm) 7.15-7.30 (m, 7H), 7.61-7.68 (m, 5H), 7.82-7.84 (m, 2H).

To a solution of Compound 86A (500 mg, 1 mmol) in 1,2-dichloroethane (20 mL) was added 3-chloroperoxybenzoic acid (220 mg, 1.2 mmol) and 4,4′-thiobis(6-tert-butyl-O-cresol) (17.9 mg, 0.05 mmol) under nitrogen. The mixture was stirred under nitrogen at 85° C. for 16 hours. After cooled down to room temperature, the mixture was washed with 10% aqueous Na₂S₂O₅ solution (20 mL) and saturated NaHCO₃ solution (20 mL) and brine (20 mL), dried over anhydrous sodium sulfate, filtered, and concentrated under reduced pressure. The crude product was purified with flash column chromatography on silica gel (ethyl acetate in petroleum ether, 20% v/v) to give Compound 86B: LC-MS (ESI) m/z: 991 [2M+Na]⁺; ¹H-NMR (CDCl₃, 400 MHz): δ (ppm) 5.21-5.48 (m, 1H), 7.18-7.33 (m, 9H), 7.40-7.81 (m, 4H).

To a solution of Compound 86B (180 mg, 0.37 mmol) in THF (20 mL) was added phenylmagnesium bromide (1 Min ether, 3.7 mL) at 0° C. under nitrogen. The mixture was stirred at 25° C. under nitrogen for 16 hours, quenched with ice-water (20 mL), and extracted with ethyl acetate (20 mL×2). The combined organic layers was washed with brine (20 mL), dried over anhydrous sodium sulfate, filtered, and concentrated to give a crude product, which was purified with preparative HPLC and chiral HPLC to yield Compound 86-1 and Compound 86-2. Compound 86-1: LC-MS (ESI) m/z: 545 [M-OH]⁺; ¹H-NMR (CDCl₃, 400 MHz): δ (ppm) 5.19-5.34 (ds, 1H), 6.78-7.07 (m, 4H), 7.22-7.26 (m, 2H), 7.31-7.41 (m, 12H); Chiral separation condition: MeOH contained 0.2% Methanol Ammonia; OJ-H (4.6*150 mm, 5 μm); retention time: 2.85 minutes (10%), 4.85 minutes (90%). Compound 86-2: LC-MS (ESI) m/z: 545 [M-OH]⁺; ¹H-NMR (CDCl₃, 400 MHz): δ (ppm) 5.23-5.34 (ds, 1H), 6.78-7.07 (m, 3H), 7.22-7.26 (m, 2H), 7.31-7.41 (m, 13H); Chiral separation condition: MeOH contained 0.2% Methanol Ammonia; OJ-H (4.6*150 mm, 5 μm); retention time: 3.63 minutes (94%), 4.78 minutes (6%).

### Example 87

### Synthesis of 1,3-bis(4-chlorophenyl)-4-hydroxy-5-propyl-4-(3-(trifluoromethyl)phenyl)imidazolidin-2-one, (5S)-1,3-bis(4-chlorophenyl)-4-hydroxy-5-propyl-4-(3-(trifluoromethyl)phenyl)imidazolidin-2-one, and (5R)-1,3-bis(4-chlorophenyl)-4-hydroxy-5-propyl-4-(3-(trifluoromethyl)phenyl)imidazolidin-2-one

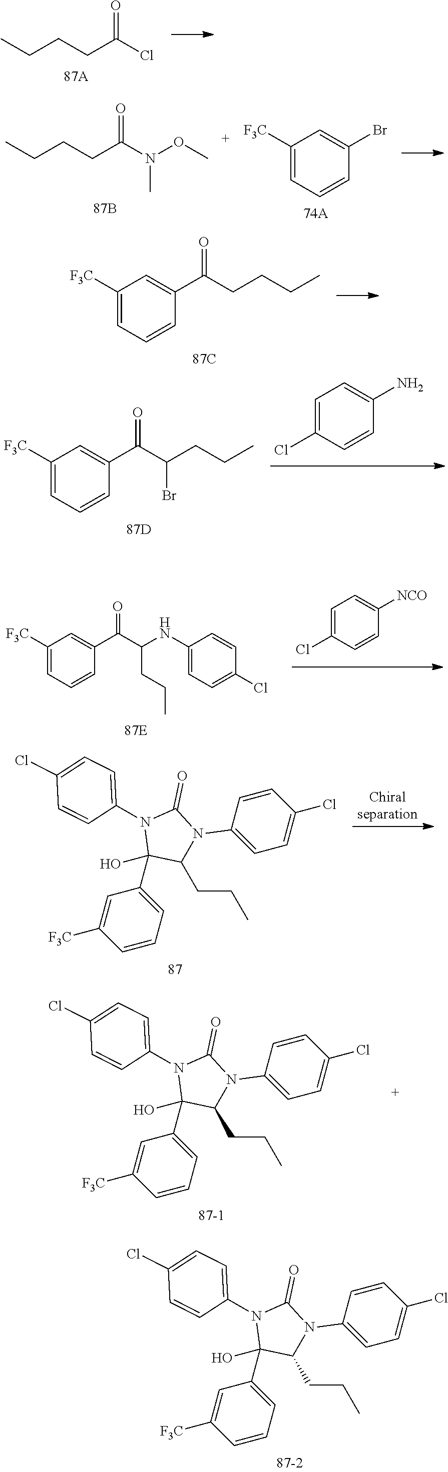

Compounds 87B, 87C, 87D, 87E, and 87 synthesized by employing the procedures described for Compounds 42B, 59B, 13B, 1B, and 1 using Compounds 87A, 87B, 74A, 87C, 87D, 4-chloroaniline, 87E, and 1-chloro-4-isocyanatobenzene in lieu of Compounds 42A, N-methoxy-N-methylacetamide, 59A, 13A, 1A, 4-bromoaniline, 1B, and 1-bromo-4-isocyanatobenzene. Compound 87B. ¹H-NMR (CDCl₃, 400 MHz): δ (ppm) 0.93 (t, J=7.2 Hz, 3H), 1.34-1.40 (m, 2H), 1.60-1.76 (m, 2H), 2.42 (t, J=7.6 Hz, 2H), 3.18 (s, 3H), 3.68 (s, 3H). Compound 87C: LC-MS (ESI) m/z: 231 [M+H]⁺; ¹H-NMR (CDCl₃, 400 MHz): δ (ppm) 0.97 (t, J=7.2 Hz, 3H), 1.40-1.45 (m, 2H), 1.70-1.78 (m, 2H), 3.00 (t, J=7.2 Hz, 2H), 7.61 (t, J=7.6 Hz, 1H), 7.81 (d, J=7.6 Hz, 1H), 8.14 (d, J=7.6 Hz, 1H), 8.21 (s, 1H). Compound 87D: LC-MS (ESI) m/z: 309 [M+H]⁺. Compound 87E: LC-MS (ESI) m/z: 356 [M+H]⁺; ¹H-NMR (CDCl₃, 400 MHz): δ (ppm) 0.91 (t, J=7.2 Hz, 3H), 1.45-1.52 (m, 2H), 1.81-2.02 (m, 2H), 4.59-4.61 (m, 1H), 4.99-5.04 (m, 1H), 6.60 (d, J=8.8 Hz, 2H), 7.12 (d, J=8.8 Hz, 2H), 7.67 (t, J=8.0 Hz, 1H), 7.88 (d, J=8.0 Hz, 1H), 8.17 (d, J=8.0 Hz, 1H), 8.24 (s, 1H). Compound 87: LC-MS (ESI) m/z: 509 [M+H]⁺; ¹H-NMR (CDCl₃, 400 MHz): δ (ppm) 0.72 (t, J 7.2 Hz, 3H), 0.84-0.99 (m, 2H), 1.69-1.84 (m, 2H), 3.67 (s, 1H), 4.23-4.26 (m, 1H), 7.14 (d, J=8.8 Hz, 2H), 7.26-7.29 (m, 4H), 7.39 (d, J=8.8 Hz, 2H), 7.46 (t, J=8.0 Hz, 1H), 7.57 (d, J=8.0 Hz, 1H), 7.72 (d, J=8.0 Hz, 1H), 7.89 (s, 1H).

Compound 87 was separated with chiral HPLC to give Compound 87-1 and Compound 87-2. Compound 87-1: LC-MS (ESI) m/z: 509 [M+H]⁺; ¹H-NMR (CDCl₃, 400 MHz): δ (ppm) 0.73 (t, J=7.2 Hz, 3H), 0.89-0.99 (m, 1H), 1.16-1.24 (m, 1H), 1.67-1.86 (m, 2H), 3.72 (s, 1H), 4.23-4.26 (m, 1H), 7.10 (d, J=9.2 Hz, 2H), 7.22 (d, J=9.2 Hz, 2H), 7.28 (d, J=8.8 Hz, 2H), 7.39 (d, J=8.8 Hz, 2H), 7.45 (t, J=8.0 Hz, 1H), 7.56 (d, J=8.4 Hz, 1H), 7.69 (d, J=8.4 Hz, 1H), 7.87 (s, 1H). Chiral separation condition: MeOH contained 0.2% Methanol Ammonia; RegisCell (4.6*250 mm, 5 μm); retention time: 2.13 minutes (98%), 3.53 minutes (2%). Compound 87-2: LC-MS (ESI) m/z: 509 [M+H]⁺; ¹H-NMR (CDCl₃, 400 MHz): δ (ppm) 0.73 (t, J=7.2 Hz, 3H), 0.91-0.99 (m, 1H), 1.16-1.24 (m, 1H), 1.68-1.85 (m, 2H), 3.72 (s, 1H), 4.22-4.26 (m, 1H), 7.10 (d, J=8.8 Hz, 2H), 7.21 (d, J=8.8 Hz, 2H), 7.28 (d, J=8.8 Hz, 2H), 7.38 (d, J=8.8 Hz, 2H), 7.45 (t, J=8.0 Hz, 1H), 7.56 (d, J=8.4 Hz, 1H), 7.69 (d, J=8.4 Hz, 1H), 7.87 (s, 1H). Chiral separation condition: co-solvent: MeOH contained 0.2% Methanol Ammonia; RegisCell (4.6*250 mm, 5 μm); retention time: 3.80 minutes (3%), 4.24 minutes (88%).

### Example 88

### Synthesis of 1,3-bis(4-bromophenyl)-4-(3-ethylphenyl)-4-hydroxy-5-propylimidazolidin-2-one, (5S)-1,3-bis(4-bromophenyl)-4-(3-ethylphenyl)-4-hydroxy-5-propylimidazolidin-2-one, and (5R)-1,3-bis(4-bromophenyl)-4-(3-ethylphenyl)-4-hydroxy-5-propylimidazolidin-2-one

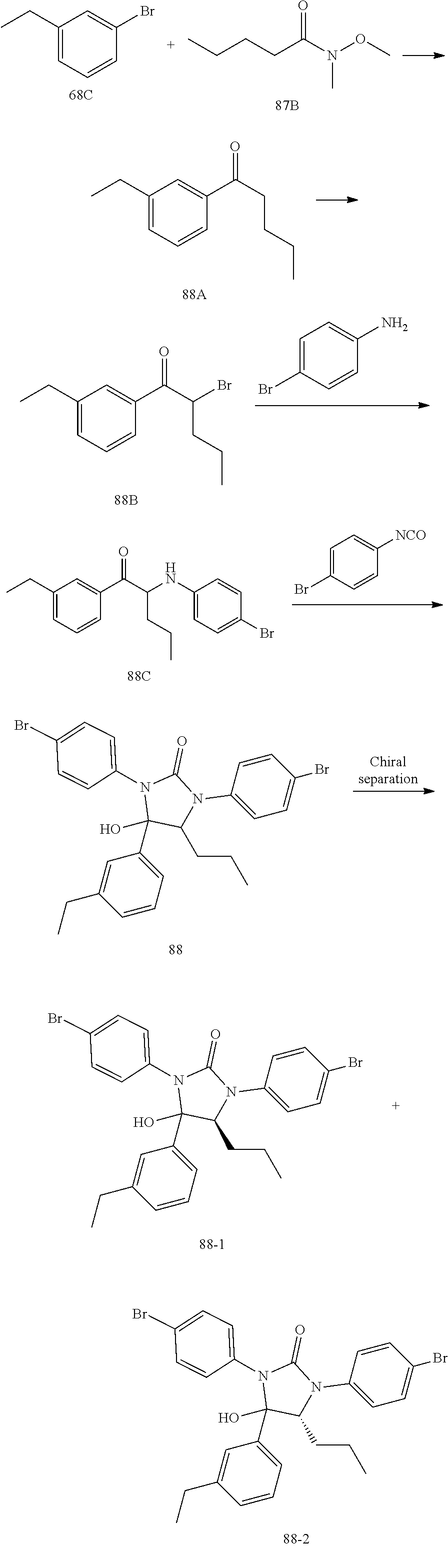

Compounds 88A, 88B, 88C, and 88 were synthesized by employing the procedures described for Compounds 59B, 13B, 1B, and 1 using Compounds 87B, 68C, 88A, 88B, heating at 60° C., and 88C in lieu of N-methoxy-N-methylacetamide, Compounds 59A, 13A, 1A, stirred at room temperature, and 1B. Compound 88A: LC-MS (ESI) m/z: 191 [M+H]⁺. Compound 88B: LC-MS (ESI) m/z: 269 [M+H]⁺; ¹H-NMR (CDCl₃, 400 MHz): δ (ppm) 0.99 (t, J=7.6 Hz, 3H), 1.28 (t, J=8.0 Hz, 3H), 1.43-1.58 (m, 2H), 2.10-2.19 (m, 2H), 2.72 (q, J=7.6 Hz, 2H), 5.16 (t, J=7.6 Hz, 1H), 7.38-7.45 (m, 2H), 7.81-7.85 (m, 2H). Compound 88C: LC-MS (ESI) m/z: 360 [M+H]⁺. Compound 88: LC-MS (ESI) m/z: 557 [M+H]⁺; ¹H-NMR (CDCl₃, 400 MHz): δ (ppm) 0.48, 0.72 (t, J=7.2 Hz, 3H), 0.99-1.07 (m, 1H), 1.12-1.26 (m, 4H), 1.63-1.82 (m, 2H), 2.59-2.64 (m, 2H), 3.41, 3.91 (s, 1H), 4.14-4.26 (m, 1H), 7.10-7.12 (m, 1H), 7.20-7.35 (m, 9H), 7.47-7.53 (m, 2H).

Compound 88 was separated with chiral-HPLC to give Compound 88-1 and Compound 88-2. Compound 88-1: LC-MS (ESI) m/z: 557 [M+H]⁺; ¹H-NMR (CDCl₃, 400 MHz): δ (ppm) 0.47, 0.70 (t, J=7.2 Hz, 3H), 0.96-1.29 (m, 5H), 1.61-1.87 (m, 2H), 2.55-2.64 (m, 2H), 3.62, 4.14 (s, 1H), 4.11, 4.23 (dd, J=9.6, 3.6 Hz, 1H), 7.09-7.13 (m, 1H), 7.16-7.38 (m, 9H), 7.43-7.52 (m, 2H). Chiral separation condition: MeOH contained 0.2% methanol ammonia; IC (4.6*150 mm, 5 μm); retention time: 1.77 minutes (78%), 2.77 minutes (22%). Compound 88-2: LC-MS (ESI) m/z: 557 [M+H]⁺; ¹H-NMR (CDCl₃, 400 MHz): δ (ppm) 0.47, 0.72 (t, J=7.2 Hz, 3H), 1.11-1.27 (m, 5H), 1.61-1.87 (m, 2H), 2.58-2.64 (m, 2H), 3.55, 4.11 (s, 1H), 4.11, 4.24 (dd, J=9.6, 3.6 Hz, 1H), 7.09-7.13 (m, 1H), 7.16-7.38 (m, 9H), 7.43-7.51 (m, 2H). Chiral separation condition: MeOH contained 0.2% methanol ammonia; IC (4.6*150 mm 5 μm); retention time: 2.27 minutes (23%), 4.48 minutes (77%).

### Example 89

### Synthesis of 1,3-bis(4-chlorophenyl)-4-(3-ethylphenyl)-4-hydroxy-5-propylimidazolidin-2-one, (5S)-1,3-bis(4-chlorophenyl)-4-(3-ethylphenyl)-4-hydroxy-5-propylimidazolidin-2-one, and (5R)-1,3-bis(4-chlorophenyl)-4-(3-ethylphenyl)-4-hydroxy-5-propylimidazolidin-2-one

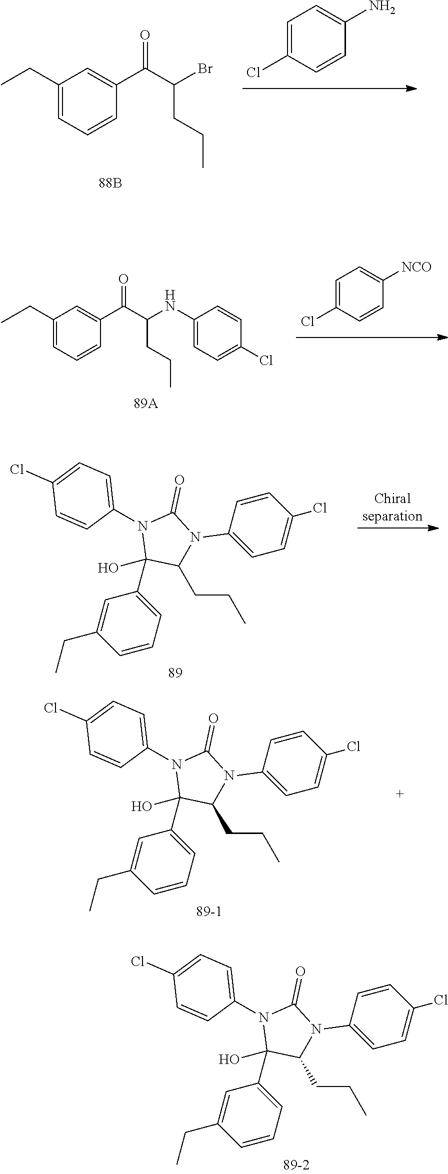

Compounds 89A and 89 were synthesized by employing the procedures described for Compounds 1B and 1 using Compound 88B, 4-chloroaniline, heating at 60° C., 89A, and 1-chloro-4-isocyanatobenzene in lieu of Compounds 1A, 4-bromoaniline, stirred at room temperature, 1B, and 1-bromo-4-isocyanatobenzene. Compound 89A: LC-MS (ESI) m/z: 316 [M+H]⁺. Compound 89: LC-MS (ESI) m/z: 469 [M+H]⁺; ¹H-NMR (CDCl₃, 400 MHz): δ (ppm) 0.46, 0.71 (t, J=7.2 Hz, 3H), 0.75-1.05 (m, 1H), 1.10-1.18 (m, 3H), 1.23-1.30 (m, 2H), 1.63-1.79 (m, 1H), 2.55-2.64 (m, 2H), 3.41, 4.08 (s, 1H), 4.12-4.25 (m, 1H), 7.08-7.12 (m, 3H), 7.18-7.43 (m, 9H) was separated with chiral HPLC to give Compound 89-1 and Compound 89-2. Compound 89-1: LC-MS (ESI) m/z: 469 [M+H]⁺; ¹H-NMR (CDCl₃, 400 MHz): δ (ppm) 0.45, 0.71 (t, J=7.2 Hz, 3H), 0.96-1.28 (m, 6H), 1.62-1.86 (m, 1H), 2.55-2.63 (m, 2H), 3.81, 4.40 (s, 1H), 4.11, 4.23 (dd, J=9.6, 3.6 Hz, 1H), 7.03-7.11 (m, 3H), 7.19-7.39 (m, 9H). Chiral separation condition: MeOH contained 0.2% methanol ammonia; OD-H (4.6*250 mm, 5 μm); retention time: 1.74 minutes (76%), 4.35 minutes (24%). Compound 89-2: LC-MS (ESI) m/z: 469 [M+H]⁺; ¹H-NMR (CDCl₃, 400 MHz): δ (ppm) 0.43-0.77 (m, 4H), 0.88-1.26 (m, 5H), 1.63-1.83 (m, 1H), 2.54-2.63 (m, 2H), 3.72, 4.45 (s, 1H), 4.11, 4.23 (dd, J=9.6, 3.6 Hz, 1H), 7.05-7.09 (m, 3H), 7.18-7.39 (m, 9H). Chiral separation condition: MeOH contained 0.2% methanol ammonia; OD-H (4.6*250 mm, 5 μm); retention time: 2.75 minutes (26%), 6.25 minutes (74%).

### Example 90

### Synthesis of 1,3-bis(4-chlorophenyl)-4-hydroxy-5-propyl-4-(m-tolyl)imidazolidin-2-one, (5S)-1,3-bis(4-chlorophenyl)-4-hydroxy-5-propyl-4-(m-tolyl)imidazolidin-2-one, and (5R)-1,3-bis(4-chlorophenyl)-4-hydroxy-5-propyl-4-(m-tolyl)imidazolidin-2-one

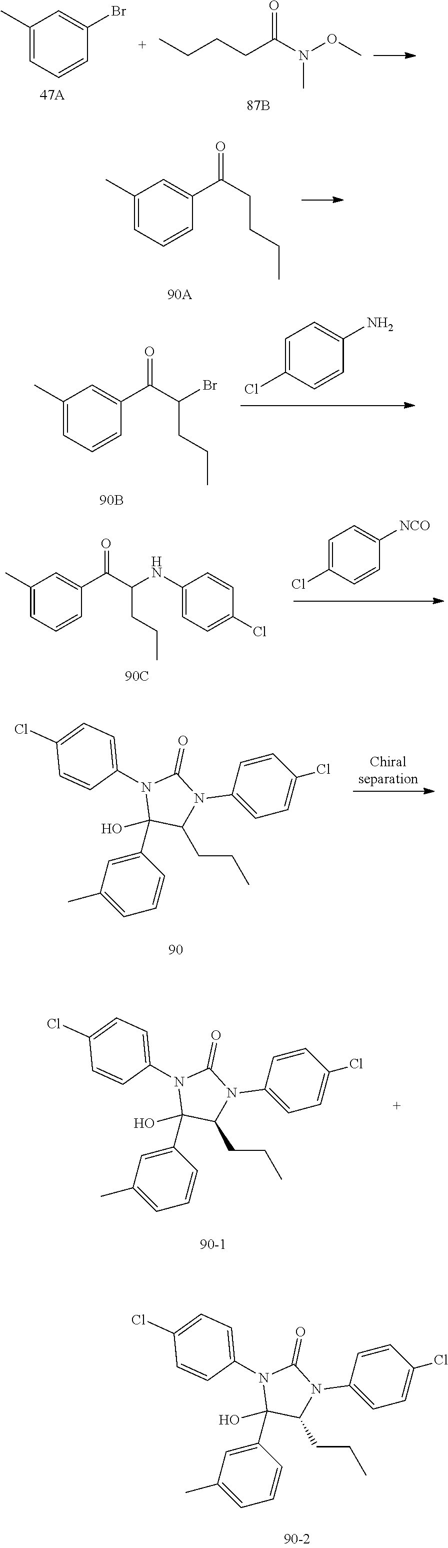

Compounds 90A, 90B, 90C, and 90 were synthesized by employing the procedures described for Compounds 59B, 13B, 1B, and 1 using Compounds 87B, 47A, 90A, 90B, 4-chloroaniline, heating at 65° C., 90C, and 1-chloro-4-isocyanatobenzene in lieu of N-methoxy-N-methylacetamide, Compounds 59A, 13A, 1A, 4-bromoaniline, stirred at room temperature, 1B, and 1-chloro-4-isocyanatobenzene. Compound 90A: LC-MS (ESI) m/z: 177 [M+H]⁺; ¹H-NMR (CDCl₃, 400 MHz): δ (ppm) 0.93 (t, J=7.2 Hz, 3H), 1.36-1.46 (m, 2H), 1.68-1.75 (m, 2H), 2.41 (s, 3H), 2.93 (t, J=8.4 Hz, 2H), 7.32-7.38 (m, 2H), 7.74-7.77 (m, 2H). Compound 90B: LC-MS (ESI) m/z: 255 [M+H]⁺; ¹H-NMR (CDCl₃, 400 MHz): δ (ppm) 0.96 (t, J=7.2 Hz, 3H), 1.31-1.49 (m, 2H), 2.09-2.22 (m, 2H), 2.41 (s, 3H), 5.13-5.17 (m, 1H), 7.35-7.42 (m, 2H), 7.80-7.82 (m, 2H). Compound 90C: LC-MS (ESI) m/z: 302 [M+H]⁺. Compound 90: LC-MS (ESI) m/z: 455 [M+H]⁺; ¹H-NMR (DMSO-d₆, 400 MHz): δ (ppm) 0.41-0.63 (m, 3H), 0.83-1.44 (m, 2H), 1.44-1.79 (m, 2H), 2.26 (s, 3H), 4.29-1.37 (m, 1H), 7.03-7.10 (m, 1H), 7.16-7.63 (m, 12H).

Compound 90 was separated with chiral HPLC to give Compound 90-1 and Compound 90-2. Compound 90-1: LC-MS (ESI) m/z: 455 [M+H]⁺; ¹H-NMR (CD₃OD, 400 MHz): δ (ppm) 0.49-0.71 (m, 3H), 0.88-1.88 (m, 4H), 2.32 (s, 3H), 4.29-4.45 (m, 1H), 7.07-7.22 (m, 4H), 7.33-7.57 (m, 8H). Chiral separation condition: MeOH contained 0.2% Methanol Ammonia; RegisCell-H (4.6*250 mm, 5 μm); retention time: 3.09 minutes (76%), 5.13 minutes (24%). Compound 90-2: LC-MS (ESI) m/z: 455 [M+H]⁺; ¹H-NMR (CD₃OD, 400 MHz): δ (ppm) 0.49-0.71 (m, 3H), 0.90-1.89 (m, 4H), 2.32 (s, 3H), 4.29-4.45 (m, 1H), 7.07-7.22 (m, 4H), 7.33-7.57 (m, 8H). Chiral separation condition: co-solvent: MeOH contained 0.2% Methanol Ammonia; RegisCell-H (4.6*250 mm, 5 μm); retention time: 3.88 minutes (20%), 6.92 minutes (76.4%).

### Example 91

### Synthesis of 1,3-bis(4-chlorophenyl)-4-hydroxy-5-propyl-4-(3-(trifluoromethoxy)phenyl)imidazolidin-2-one, (5S)-1,3-bis(4-chlorophenyl)-4-hydroxy-5-propyl-4-(3-(trifluoromethoxy)phenyl)imidazolidin-2-one, and (5R)-1,3-bis(4-chlorophenyl)-4-hydroxy-5-propyl-4-(3-(trifluoromethoxy)phenyl)imidazolidin-2-one

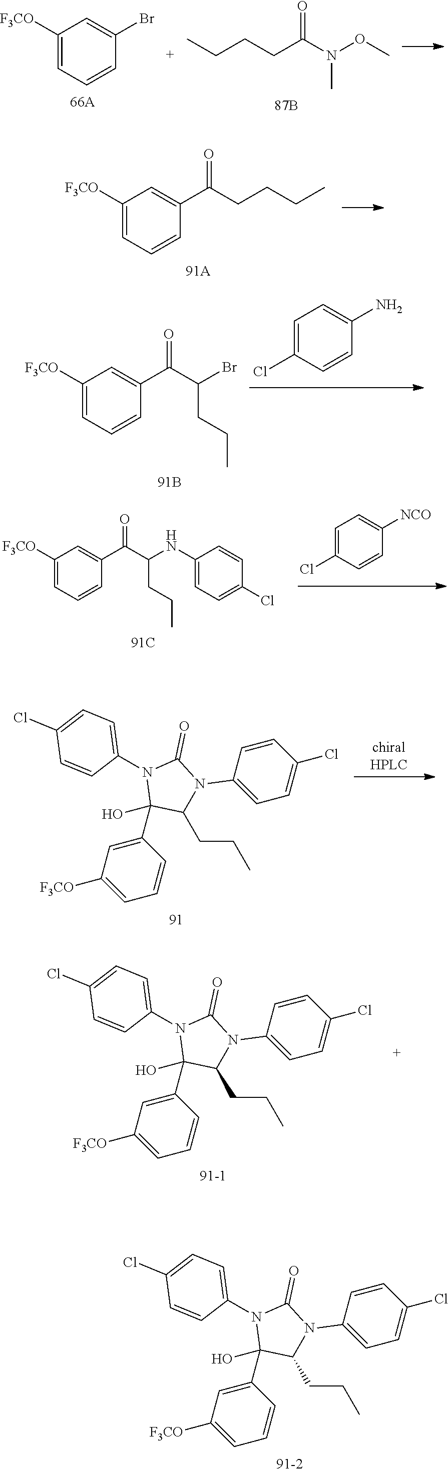

Compounds 91A, 91B, 91C, and 91 were synthesized by employing the procedures described for Compounds 59B, 13B, 1B, and 1 using Compounds 87B, 66A, 91A, 91B, 4-chloroaniline, heating at 60° C., 91C, and 1-chloro-4-isocyanatobenzene in lieu of N-methoxy-N-methylacetamide, Compounds 59A, 13A, 1A, 4-bromoaniline, stirred at room temperature, 1B, and 1-chloro-4-isocyanatobenzene. Compound 91A: LC-MS (ESI) m/z: non-ionizable compound under routine conditions used; ¹H-NMR (CDCl₃, 400 MHz): δ (ppm) 0.96 (t, J=7.6 Hz, 3H), 1.39-1.44 (m, 2H), 1.71-1.75 (m, 2H), 2.96 (t, J=7.2 Hz, 2H), 7.40-7.42 (m, 1H), 7.51 (t, J=8.0 Hz, 1H), 7.80 (s, 1H), 7.89 (d, J=7.6 Hz, 1H). Compound 91B: LC-MS (ESI) m/z: non-ionizable compound under routine conditions used; ¹H-NMR (CDCl₃, 400 MHz): δ (ppm) 1.00 (t, J=7.6 Hz, 3H), 1.43-1.56 (m, 2H), 2.11-2.21 (m, 2H), 5.06-5.09 (m, 1H), 7.44-7.46 (m, 1H), 7.54 (t, J=8.0 Hz, 1H), 7.86 (s, 1H), 7.95 (d, J=7.6 Hz, 1H).

Compound 91C: LC-MS (ESI) m/z: 372 [M+H]⁺.

Compound 91 was separated with chiral HPLC to give Compound 91-1 and Compound 91-2. Compound 91-1: LC-MS (ESI) m/z: 525 [M+H]⁺; ¹H-NMR (CDCl₃, 400 MHz): δ (ppm) 0.48, 0.72 (t, J=7.6 Hz, 3H), 0.92-1.25 (m, 2H), 1.65-1.86 (m, 2H), 4.18-4.25 (m, 2H), 7.03-7.25 (m, 6H), 7.27-7.48 (m, 6H). Chiral separation condition: MeOH contained 0.2% methanol ammonia; RegisCell (4.6*250 mm, 5 μm); retention time: 2.71 minutes (80.7%), 5.40 minutes (19.3%). Compound 91-2: LC-MS (ESI) m/z: 525 [M+H]⁺; ¹H-NMR (CDCl₃, 400 MHz): δ (ppm) 0.48, 0.72 (t, J=7.6 Hz, 3H), 0.92-1.25 (m, 2H), 1.65-1.86 (m, 2H), 4.16-4.23 (m, 2H), 7.03-7.25 (m, 6H), 7.27-7.46 (m, 6H). Chiral separation condition: MeOH contained 0.2% methanol ammonia; RegisCell (4.6*250 mm, 5 μm); retention time: 4.04 minutes (8.1%), 6.45 minutes (89.8%).

### Example 92

### Synthesis of 1,3-bis(4-chlorophenyl)-4-hydroxy-5-(methoxymethyl)-4-(3-(trifluoromethyl)phenyl)imidazolidin-2-one, (5S)-1,3-bis(4-chlorophenyl)-4-hydroxy-5-(methoxymethyl)-4-(3-(trifluoromethyl)phenyl)imidazolidin-2-one, and (5R)-1,3-bis(4-chlorophenyl)-4-hydroxy-5-(methoxymethyl)-4-(3-(trifluoromethyl)phenyl)imidazolidin-2-one

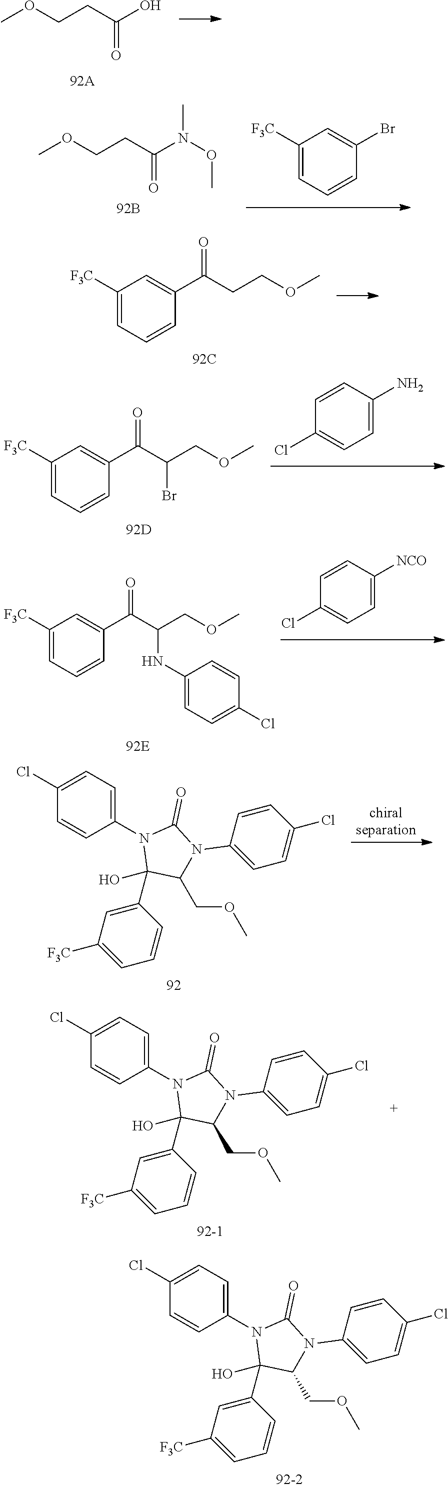

To a solution of 3-methoxypropanoic acid 92A (5.00 g, 48.08 mmol) in dichloromethane (50 mL) and DMF (0.5 mL) was dropped oxalyl dichloride (9.09 g, 72.12 mmol) at 0° C. The mixture was stirred at 25° C. for 16 hours and concentrated to give a crude product (5.51 g, yield 94%). The crude 3-methoxypropenoyl chloride (5.51 g, 45.20 mmol) was added dropwise to a suspension of N,O-dimethylhydroxylamine hydrochloride (6.58 g, 67.80 mmol) and Et₃N (9.94 g, 90.40 mmol) in dichloromethane (100 mL) at room temperature. The mixture was stirred at 25° C. for 3 hours and filtered. The filtrate was concentrated and the residue was purified with flash column chromatography in silica gel (ethyl acetate in petroleum ether, from 0% to 30% v/v) to give Compound 92B. LC-MS (ESI) m/z: non-ionizable compound under routine conditions used; ¹H-NMR (CDCl₃, 400 MHz): δ (ppm) 2.72 (d, J=6.4 Hz, 2H), 3.19 (s, 3H), 3.37 (s, 3H), 3.68-3.72 (m, 5H).

Compounds 92C, 92D, 92E, and 92 were synthesized by employing the procedures described for Compounds 59B, 13B, 1B, and 1 using 1-bromo-3-(trifluoromethyl)benzene, Compounds 92B, 92C, 92D, 4-chloroaniline using NMP as solvent at 55° C., 1-chloro-4-isocyanatobenzene, and Compound 92E in lieu of Compound 59A, N-methoxy-N-methylacetamide, Compounds 13A, 1A, 4-bromoaniline using EtOH as solvent at 25° C., 1-bromo-4-isocyanatobenzene, and Compound 1B. Compound 92C. LC-MS (ESI) m/z: 233 [M+H]⁺; ¹H-NMR (CDCl₃, 400 MHz): δ (ppm) 3.26 (t, J=6.4 Hz, 2H), 3.64 (s, 3H), 3.83 (t, J=6.4 Hz, 2H), 7.62 (t, J=8.0 Hz, 1H), 7.82 (t, J=8.0 Hz, 1H), 8.15 (d, J=8.0 Hz, 1H), 8.23 (s, 1H). Compound 92D. LC-MS (ESI) m/z: 311 [M+H]⁺; ¹H-NMR (CDCl₃, 400 MHz): δ (ppm) 3.44 (s, 3H), 3.86-3.90 (m, 1H), 4.11-4.16 (m, 1H), 5.16-5.20 (m, 1H), 7.65 (t, J=8.0 Hz, 1H), 7.86 (d, J=8.0 Hz, 1H), 8.19 (d, J=7.6 Hz, 1H), 8.27 (s, 1H). Compound 92E. LC-MS (ESI) m/z: 358 [M+H]⁺; ¹H-NMR (CDCl₃, 400 MHz): δ (ppm) 3.28 (s, 3H), 3.72-3.78 (m, 2H), 4.81 (d, J=8.0 Hz, 1H), 5.06-5.11 (m, 1H), 6.62 (d, J=8.8 Hz, 2H), 7.13 (d, J=8.8 Hz, 2H), 77.65 (t, J=8.0 Hz, 1H), 77.87 (d, J=7.6 Hz, 1H), 8.19 (d, J=8.0 Hz, 1H), 8.27 (s, 1H).

Compound 92 was separated with chiral HPLC to give Compound 92-1 and Compound 92-2. Compound 92-1: LC-MS (ESI) m/z: 511 [M+H]⁺; ¹H-NMR (CDCl₃, 400 MHz): δ (ppm) 2.64, 3.31 (s, 3H), 2.62-2.65, 3.84-3.87 (m, 1H), 2.90-2.94, 3.60-3.64 (m, 1H), 4.17-4.24 (m, 1H), 5.49, 5.51 (s, 1H), 7.00-7.23 (m, 3H), 7.30-7.91 (m, 9H). Chiral separation condition: MeOH contained 0.2% Methanol ammonia; OD-H (4.6*100 mm, 5 μm); retention time: 1.67 minute (71%), 4.52 minute (29%). Compound 92-2: LC-MS (ESI) m/z: 511[M+H]⁺; ¹H-NMR (CDCl₃, 400 MHz): δ (ppm) 2.66, 3.31 (s, 3H), 2.65-2.68, 3.84-3.87 (m, 1H), 2.92-2.97, 3.61-3.64 (m, 1H), 4.19-4.25 (m, 1H), 5.11, 5.81 (s, 1H), 7.00-7.23 (m, 3H), 7.33-7.91 (m, 9H); Chiral separation condition: MeOH contained 0.2% Methanol ammonia; OD-H (4.6*100 mm, 5 μm); retention time: 2.79 minute (32%), 3.76 minute (68%).

### Example 93

### Synthesis of 1,3-bis(4-chlorophenyl)-4-(3-ethynylphenyl)-4-hydroxy-5-methylimidazolidin-2-one, (5S)-1,3-bis(4-chlorophenyl)-4-(3-ethynylphenyl)-4-hydroxy-5-methylimidazolidin-2-one, and (5R)-1,3-bis(4-chlorophenyl)-4-(3-ethynylphenyl)-4-hydroxy-5-methylimidazolidin-2-one

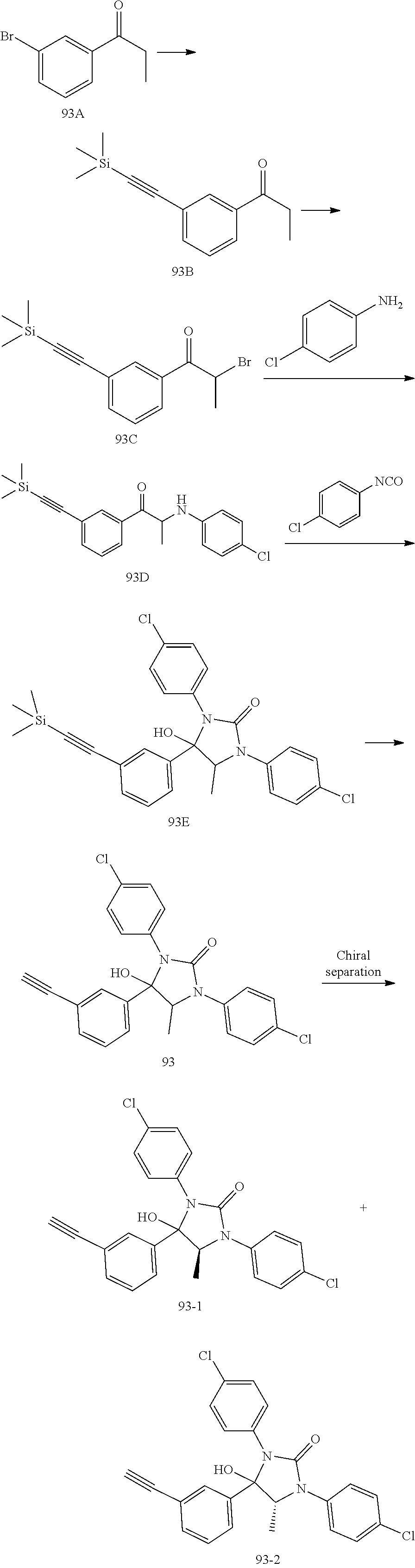

A solution of 1-(3-bromophenyl)propan-1-one 93A (5 g, 23.6 mmol), ethynyltrimethylsilane (3.5 g, 35.3 mmol), PdCl₂(PPh₃)₂ (0.95 g, 1.2 mmol), and copper (I) iodide (0.2 g, 1.2 mmol) in triethylamine (50 mL) was stirred at 50° C. under nitrogen for 16 hours. The mixture was cooled down to room temperature and filtered. The organic phase was diluted with ethyl acetate (100 mL), washed with water (30 mL×2) and brine (30 mL), dried over anhydrous sodium sulfate, filtered, and concentrated under reduced pressure. The residue was purified with flash column chromatography on silica gel (ethyl acetate in petroleum ether, 5% v/v) to furnish Compound 93B: LC-MS (ESI) m/z: 231 [M+H]⁺; ¹H-NMR (CDCl₃, 400 MHz): δ (ppm) 0.27 (t, J=3.6 Hz, 9H), 1.22 (t, J=7.2 Hz, 3H), 3.00 (q, J=7.2 Hz, 2H), 7.40 (t, J=7.2 Hz, 1H), 7.63 (d, J=7.2 Hz, 1H), 7.90 (d, J=8.0 Hz, 1H), 8.04 (s, 1H).

A mixture of Compound 93B (1 g, 4.3 mmol) and copper (II) bromide (1.9 g, 8.7 mmol) in ethyl acetate (10 mL) and chloroform (10 mL) was stirred at 60° C. under nitrogen for 3 hours. The mixture was cooled down to room temperature and filtered. The organic phase was diluted with ethyl acetate (30 mL), washed with water (20 mL×2) and brine (20 mL), dried over anhydrous sodium sulfate, filtered, and concentrated to give a crude Compound 93C, which was used directly in the next step without further purification. LC-MS (ESI) m/z: non-ionizable compound under routine conditions used.

Compound 93D and 93E were synthesized by employing the procedures described for Compounds 1B and 1 using Compounds 93C, 4-chloroaniline, heating at 60° C., 93D, and 1-chloro-4-isocyanatobenzene in lieu of Compounds 1A, 4-bromoaniline, stirred at room temperature, 1B, and 1-chloro-4-isocyanatobenzene. Compound 93D: LC-MS (ESI) m/z: 356 [M+H]⁺; ¹H-NMR (CD₃OD, 400 MHz): δ (ppm) 0.26 (t, J=3.6 Hz, 9H), 1.44 (d, J=6.8 Hz, 3H), 5.16 (q, J=7.2 Hz, 1H), 6.64 (d, J=4.8 Hz, 2H), 7.06 (d, J=4.8 Hz, 2H), 7.52 (t, J=8.0 Hz, 1H), 7.68 (d, J=7.6 Hz, 1H), 8.04 (d, J=7.6 Hz, 1H), 8.10 (s, 1H). Compound 93E: LC-MS (ESI) m/z: 509 [M+H]⁺; ¹H-NMR (CD₃OD, 400 MHz): δ (ppm) 0.22 (t, J=3.6 Hz, 9H), 1.17 (d, J=6.4 Hz, 3H), 4.39 (q, J=6.4 Hz, 1H), 7.17-7.18 (m, 2H), 7.19-7.71 (m, 9H), 7.71 (s, 1H).

A mixture of Compound 93E (194 mg, 0.38 mmol) and K₂CO₃ (26 mg, 0.19 mmol) in methanol (5 mL) was stirred at room temperature overnight. The mixture was evaporated and the residue was purified with preparative HPLC to furnish Compound 93: LC-MS (ESI) m/z: 437 [M+H]⁺; ¹H-NMR (CD₃OD, 400 MHz): δ (ppm) 0.75, 1.19 (d, J 6.4 Hz, 3H), 3.50 (d, J=2.4 Hz, 1H), 4.40-4.50 (m, 1H), 7.19-7.21 (m, 2H), 7.31-7.45 (m, 9H), 7.75 (s, 1H).

Compound 93 was separated with chiral HPLC to give Compound 93-1 and Compound 93-2. Compound 93-1: LC-MS (ESI) m/z: 437 [M+H]⁺; ¹H-NMR (CD₃OD, 400 MHz): δ (ppm) 0.73, 1.18 (d, J=6.4 Hz, 3H), 3.36 (s, 1H), 4.39-4.40 (m, 1H), 7.18-7.32 (m, 3H), 7.36-7.59 (m, 8H), 7.76 (s, 1H); Chiral separation condition: MeOH contained 0.2% Methanol Ammonia; Regiscell (4.6*250 mm, 5 μm); retention time: 2.2 minutes (82%), 3.3 minutes (17%). Compound 93-2: LC-MS (ESI) m/z: 437 [M+H]⁺; ¹H-NMR (CD₃OD, 400 MHz): δ (ppm) 0.68, 1.13 (d, J 6.4 Hz, 3H), 3.28 (t, J 1.6 Hz, 1H), 4.33-4.35 (m, 1H), 7.12-7.25 (m, 3H), 7.32-7.40 (m, 8H), 7.72 (s, 1H); Chiral separation condition: MeOH contained 0.2% Methanol Ammonia; Regiscell (4.6*250 mm, 5 μm); retention time: 2.97 minutes (16%), 4.97 minutes (79%).

### Example 94

### Synthesis of 4-(3-bromophenyl)-1,3-bis(4-chlorophenyl)-4-hydroxy-5-methylimidazolidin-2-one, (5S)-4-(3-bromophenyl)-1,3-bis(4-chlorophenyl)-4-hydroxy-5-methylimidazolidin-2-one, and (5R)-4-(3-bromophenyl)-1,3-bis(4-chlorophenyl)-4-hydroxy-5-methylimidazolidin-2-one

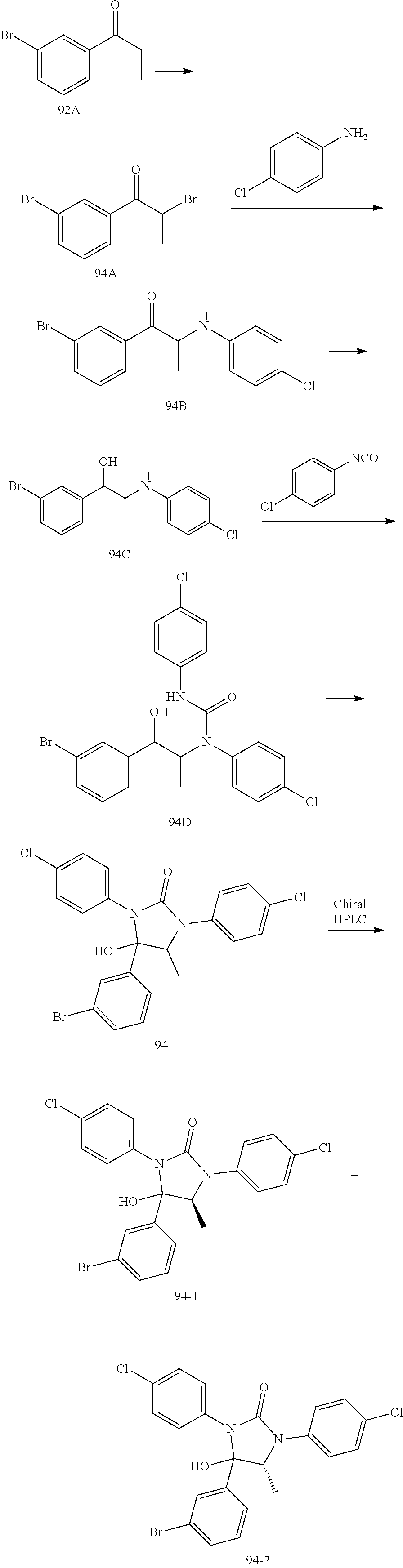

Compounds 94A, 94B, 94C, 94D, and 94 were synthesized by employing the procedures described for Compounds 13B, 13C, 14C, 1, and 14 using Compounds 92A, 94A, 4-chloroaniline, 94B, 94C, 1-chloro-4-isocyanatobenzene, and 94D in lieu of Compounds 13A, 13B, 4-bromoaniline, 14B, 1B, 1-bromo-4-isocyanatobenzene, and 14D. Compound 94A: LC-MS (ESI) m/z: 291 [M+H]⁺. Compound 94B: LC-MS (ESI) m/z: 338 [M+H]⁺. Compound 94C. LC-MS (ESI) m/z: 340 [M+H]⁺. Compound 94D: LC-MS (ESI) m/z: 493 [M+H]⁺. Compound 94: LC-MS (ESI) m/z: 491 [M+H]⁺; ¹H-NMR (DMSO-d₆, 400 MHz): δ (ppm) 1.27 (d, J=6.4 Hz, 3H), 4.36-4.42 (m, 1H), 7.26-7.32 (m, 3H), 7.40-7.50 (m, 8H), 7.57-7.62 (m, 1H), 7.76-7.79 (m, 1H).

Compound 94 was separated chiral HPLC to give Compound 94-1 and Compound 94-2. Compound 94-1: LC-MS (ESI) m/z: 491 [M+H]⁺; ¹H-NMR (acetone-d₆, 400 MHz): δ (ppm) 1.27 (d, J=4.8 Hz, 3H), 4.47-4.53 (m, 1H), 6.60 (s, 1H), 7.19-7.23 (m, 2H), 7.31 (t, J=6.4 Hz, 1H), 7.39-7.46 (m, 4H), 7.46-7.54 (m, 3H), 7.67-7.71 (m, 1H), 7.91 (t, J=1.2 Hz, 1H). Chiral separation condition: MeOH contained 0.2% Methanol Ammonia; IC (4.6*250 mm, 5 μm); retention time: 2.86 minutes (81%), 3.79 minutes (19%). Compound 94-2: LC-MS (ESI) m/z: 491 [M+H]⁺; ¹H-NMR (acetone-d₆, 400 MHz): δ (ppm) 1.27 (d, J=5.6 Hz, 3H), 4.48-4.53 (m, 1H), 6.57 (s, 1H), 7.19-7.24 (m, 2H), 7.31 (t, J=6.4 Hz, 1H), 7.39-7.54 (m, 7H), 7.67-7.72 (m, 1H), 7.91 (t, J=1.2 Hz, 1H). Chiral separation condition MeOH contained 0.2% Methanol Ammonia; IC (4.6*250 mm, 5 μm); retention time: 3.53 minutes (10%), 5.89 minutes (90%).

### Example 95

### Synthesis of 3-(1,3-bis(4-chlorophenyl)-4-hydroxy-2-oxoimidazolidin-4-yl)-5-chlorobenzonitrile

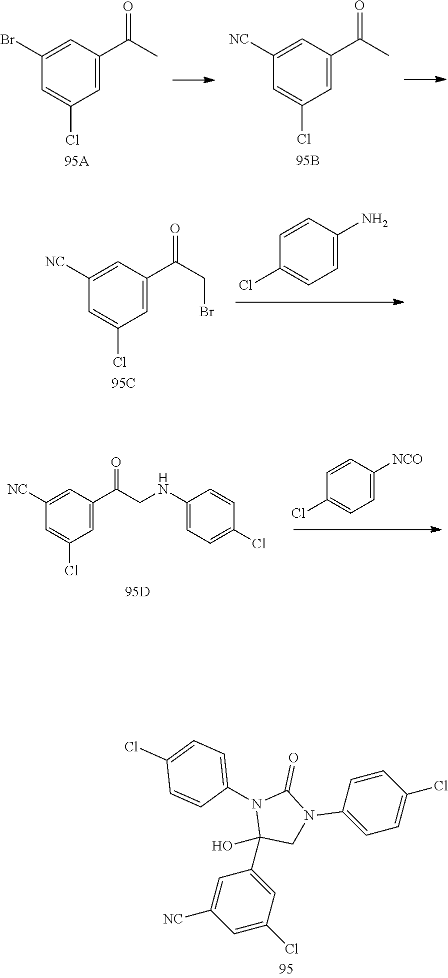

To a solution of 1-(3-bromo-5-chlorophenyl) ethanone 95A (2.32 g, 10 mmol) in N-methyl-2-pyrrolidone (10 mL) was added CuCN (1.1 g, 12 mmol) and heated at 150° C. in a microwave oven for 6 hours. After cooled down to room temperature, the mixture was diluted with ethyl acetate (40 mL) and filtered. The filtrate was washed with water (20 mL×2) and brine (20 mL), dried over anhydrous sodium sulfate, filtered, and concentrated to give a crude product, which was purified with flash column chromatography on silica gel (ethyl acetate in petroleum ether, from 0% to 20% v/v) to afford Compound 95B: LC-MS (ESI) m/z: non-ionizable compound under routine conditions used; ¹H-NMR (CDCl₃, 400 MHz): δ (ppm) 2.63 (s, 3H), 7.82-7.83 (m, 1H), 8.10-8.11 (m, 1H), 8.13-8.14 (m, 1H).

Compounds 95C, 95D, and 95 were synthesized by employing the procedures described for Compounds 13B, 1B, and 1 using Compounds 95B, 95C, 4-chloroaniline, 95D, and 1-chloro-4-isocyanatobenzene in lieu of Compounds 13A, 1A, 4-bromoaniline, 1B, and 1-bromo-4-isocyanatobenzene. Compound 95C: LC-MS (ESI) m/z: 258 [M+H]⁺.

Compound 95D: LC-MS (ESI) m/z: 305 [M+H]⁺; ¹H-NMR (DMSO-d₆, 400 MHz): δ (ppm) 4.72-4.73 (m, 2H), 6.15-6.17 (m, 1H), 6.71 (d, J=8.8 Hz, 2H), 7.08 (d, J=8.8 Hz, 2H), 8.33-8.36 (m, 2H), 8.50 (s, 1H). Compound 95: LC-MS (ESI) m/z: 458 [M+H]⁺; ¹H-NMR (CDCl₃, 400 MHz): δ (ppm) 3.83 (d, J=10.8 Hz, 1H), 4.02 (d, J=10.8 Hz, 1H), 5.43 (brs, 1H), 7.03 (d, J=8.8 Hz, 2H), 7.15 (d, J=8.8 Hz, 2H), 7.20 (d, J=8.8 Hz, 2H), 7.31 (d, J=8.8 Hz, 2H), 7.51-7.52 (m, 1H), 7.64-7.68 (m, 2H).

### Example 96

### Synthesis of 1,3-bis(4-bromophenyl)-4-hydroxy-5-propyl-4-(m-tolyl)imidazolidin-2-one, (5S)-1,3-bis(4-bromophenyl)-4-hydroxy-5-propyl-4-(m-tolyl)imidazolidin-2-one, and (5R)-1,3-bis(4-bromophenyl)-4-hydroxy-5-propyl-4-(m-tolyl)imidazolidin-2-one

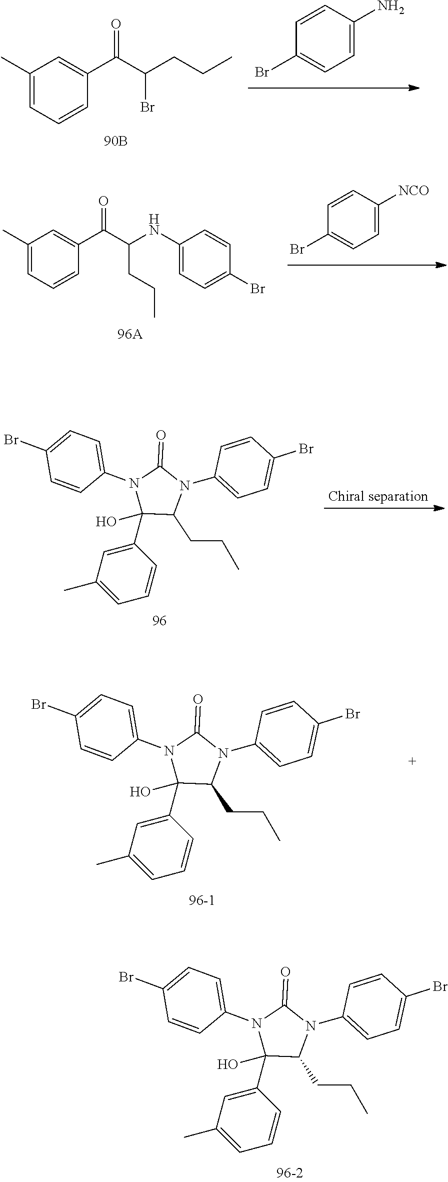

Compounds 96A and 96 were synthesized by employing the procedures described for Compounds 1B and 1 using Compounds 90B, heating at 65° C., and 96A in lieu of Compounds 1A, stirred at room temperature, and 1B. Compound 96A: LC-MS (ESI) m/z: 346 [M+H]⁺; ¹H-NMR (CDCl₃, 400 MHz): δ (ppm) 0.88 (t, J=7.2 Hz, 3H), 1.30-1.46 (m, 2H), 1.59-1.68 (m, 1H), 1.89-1.97 (m, 1H), 2.44 (s, 3H), 4.67 (d, J=7.6 Hz, 1H), 4.99-5.04 (m, 1H), 6.53-6.57 (m, 2H), 7.21-7.25 (m, 2H), 7.36-7.44 (m, 2H), 7.77-7.78 (m, 2H). Compound 96: LC-MS (ESI) m/z: 543 [M+H]⁺; ¹H-NMR (CDCl₃, 400 MHz): δ (ppm) 0.49-0.75 (m, 3H), 0.98-1.29 (m, 2H), 1.61-1.84 (m, 2H), 2.33-2.43 (m, 3H), 3.11-3.48 (m, 1H), 4.15-4.27 (m, 1H), 7.09-7.25 (m, 5H), 7.27-7.53 (m, 7H).

Compound 96 was separated with chiral HPLC to yield Compound 96-1 and Compound 96-2. Compound 96-1: LC-MS (ESI) m/z: 543 [M+H]⁺; ¹H-NMR (CDCl₃, 400 MHz): δ 0.47-0.74 (m, 3H), 0.96-1.24 (m, 2H), 1.63-1.85 (m, 2H), 2.32-2.44 (m, 3H), 3.45-3.48 (m, 1H), 4.11-4.26 (m, 1H), 7.07-7.24 (m, 7H), 7.27-7.51 (m, 5H). Chiral separation condition: MeOH contained 0.2% Methanol Ammonia; RegisCell (4.6*250 mm 5 μm); retention time: 2.20 minutes (78%), 3.36 minutes (22%). Compound 96-2: LC-MS (ESI) m/z: 543 [M+H]⁺; ¹H-NMR (CDCl₃, 400 MHz): δ 0.47-0.74 (m, 3H), 0.96-1.27 (m, 2H), 1.63-1.85 (m, 2H), 2.32-2.44 (m, 3H), 3.45-3.48 (m, 1H), 4.11-4.26 (m, 1H), 7.07-7.24 (m, 7H), 7.27-7.51 (m, 5H). Chiral separation condition: MeOH contained 0.2% Methanol Ammonia; RegisCell (4.6*250 mm 5 μm); retention time: 4.73 minutes (78%), 2.73 minutes (22%).

### Example 97

### Synthesis of 1,3-bis(4-bromophenyl)-4-hydroxy-5-methyl-4-(4-methylthiophen-2-yl)imidazolidin-2-one, (5S)-1,3-bis(4-bromophenyl)-4-hydroxy-5-methyl-4-(4-methylthiophen-2-yl)imidazolidin-2-one, and (5R)-1,3-bis(4-bromophenyl)-4-hydroxy-5-methyl-4-(4-methylthiophen-2-yl)imidazolidin-2-one

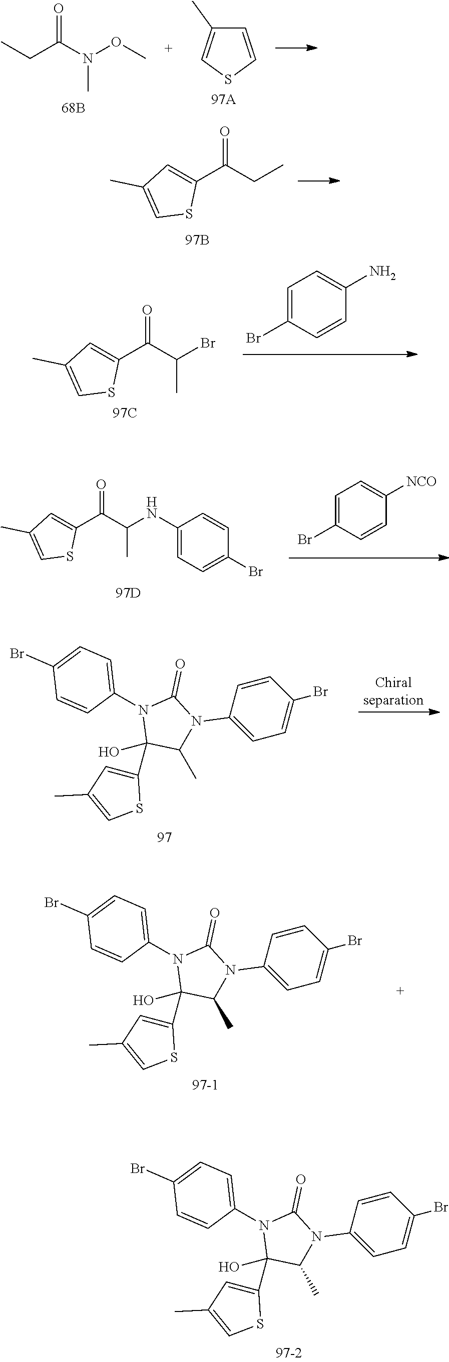

Compounds 97B, 97C, 97D, and 97 were synthesized by employing the procedures described for Compounds 59B, 13B, 1B, and 1 using Compounds 68B, 97A, 97B, 97C, heating at 60° C., and 97D in lieu of N-methoxy-N-methylacetamide, Compounds 59A, 13A, 1A, stirred at room temperature, and 1B. Compound 97B: LC-MS (ESI) m/z: non-ionizable compound under routine conditions used; ¹H-NMR (CDCl₃, 400 MHz): δ (ppm) 1.23 (t, J=7.2 Hz, 3H), 2.30 (s, 3H), 2.90 (q, J=7.2 Hz, 2H), 7.21 (s, 1H), 7.52 (s, 1H). Compound 97C: LC-MS (ESI) m/z: non-ionizable compound under routine conditions used; ¹H-NMR (CDCl₃, 400 MHz): δ (ppm) 1.87 (d, J=6.8 Hz, 3H), 2.24 (s, 3H), 5.03 (q, J=6.8 Hz, 1H), 7.30 (s, 1H), 7.51 (s, 1H). Compound 97D: LC-MS (ESI) m/z: 324 [M+H]⁺; ¹H-NMR (CDCl₃, 400 MHz): δ (ppm) 1.50 (d, J=7.2 Hz, 3H), 2.32 (s, 3H), 4.83-4.85 (m, 1H), 6.55 (d, J=8.8 Hz, 2H), 7.17 (d, J=8.8 Hz, 2H), 7.47 (s, 1H), 7.92 (s, 1H). Compound 97: LC-MS (ESI) m/z: 521 [M+H]⁺; ¹H-NMR (CDCl₃, 400 MHz): δ (ppm) 0.93, 1.31 (d, J=6.8 Hz, 3H), 3.19 (s, 3H), 3.29, 3.92 (m, 1H), 4.37-4.72 (m, 1H), 6.86-6.88 (m, 2H), 7.21 (d, J=8.8 Hz, 2H), 7.31 (d, J=8.8 Hz, 2H), 7.34 (d, J=8.8 Hz, 2H), 7.51 (d, J=8.8 Hz, 2H).

Compound 97 was separated with chiral HPLC to give Compound 97-1 and Compound 97-2. Compound 97-1: LC-MS (ESI) m/z: 521 [M+H]⁺; ¹H-NMR (CDCl₃, 400 MHz): δ (ppm) 0.84, 1.28 (d, J=6.8 Hz, 3H), 2.13, 2.16 (s, 3H), 3.92, 4.85 (s, 1H), 4.24, 4.30 (q, J=7.2 Hz, 1H), 6.60, 6.83 (s, 2H), 7.16-7.24 (m, 6H), 7.30, 7.46 (d, J=8.8 Hz, 2H); Chiral separation condition: MeOH contained 0.2% Methanol Ammonia; OZ-H (4.6*250 mm, 5 μm); retention time: 2.98 minutes (77%), 4.07 minutes (23%). Compound 97-2: LC-MS (ESI) m/z: 521 [M+H]⁺; ¹H-NMR (CDCl₃, 400 MHz): δ (ppm) 0.83, 1.28 (d, J 6.8 Hz, 3H), 2.13, 2.17 (s, 3H), 4.07, 5.05 (s, 1H), 4.24, 4.36 (q, J=7.2 Hz, 1H), 6.60, 6.83 (s, 2H), 7.16-7.23 (m, 6H), 7.30, 7.46 (d, J=8.8 Hz, 2H); Chiral separation condition: MeOH contained 0.2% Methanol Ammonia; OZ-H (4.6*250 mm, 5 μm); retention time: 4.24 minutes (23%), 5.92 minutes (77%).

### Example 98

### Synthesis of 4-(3-bromo-5-chlorophenyl)-1,3-bis(4-chlorophenyl)-4-hydroxyimidazolidin-2-one

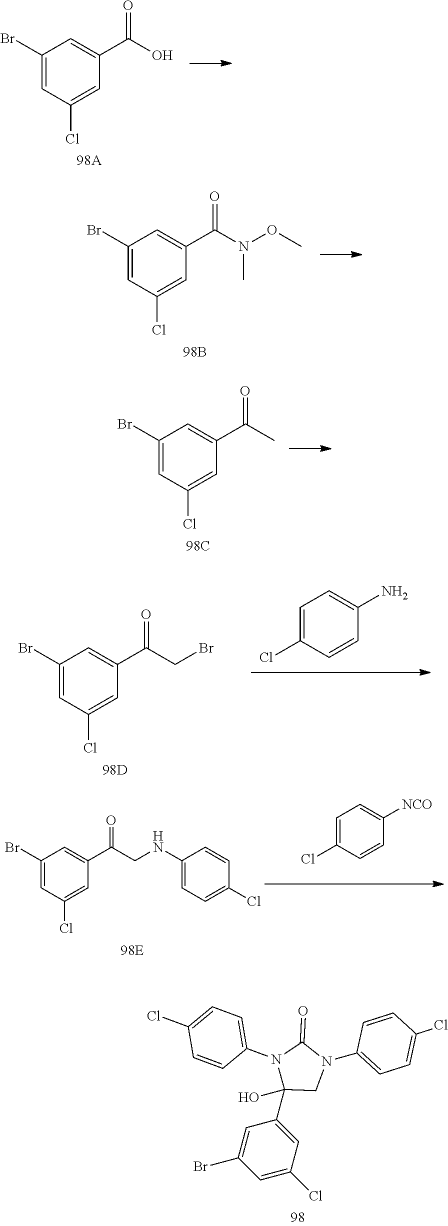

To a mixture of 3-bromo-5-chlorobenzoic acid 98A (2.0 g, 8.5 mmol), N,O-dimethylhydroxylamine hydrochloride (1.2 g, 12.8 mmol), and EDCI (2.5 g, 12.8 mmol) in dichloromethane (40 mL) was dropped triethylamine (2.6 g, 25.5 mmol) at 0° C. The mixture was stirred at 25° C. for 16 hours and diluted with dichloromethane (50 mL). The organic layer was washed with water (30 mL×2) and brine (30 mL), dried over anhydrous sodium sulfate, filtered, and concentrated to give a crude Compound 98B. LC-MS (ESI) m/z: 278 [M+H]⁺; ¹H-NMR (CDCl₃, 400 MHz): δ (ppm) 3.36 (s, 3H), 3.56 (s, 3H), 7.60-7.62 (m, 2H), 7.72 (s, 1H).

Compounds 98C, 98D, 98E, and 98 were synthesized by employing the procedures described for Compounds 42C, 13B, 1B, and 1 using Compounds 98B, MeMgBr, 98C, 98D, 4-chloroaniline, 98E, and 1-chloro-4-isocyanatobenzene in lieu of Compounds 42B, EtMgBr, 13A, 1A, 4-bromoaniline, 1B, and 1-bromo-4-isocyanatobenzene. Compound 98C: LC-MS (ESI) m/z: 233 [M+H]⁺; ¹H-NMR (CDCl₃, 400 MHz): δ (ppm) 2.59 (s, 3H), 7.71 (t, J=1.6 Hz, 1H), 7.85 (t, J=1.6 Hz, 1H), 7.96 (t, J=1.6 Hz, 1H). Compound 98D: LC-MS (ESI) m/z: 311 [M+H]⁺; ¹H-NMR (CDCl₃, 400 MHz): δ (ppm) 4.38 (s, 2H), 7.75 (t, J=2.0 Hz, 1H), 7.88 (t, J=2.0 Hz, 1H), 7.99 (t, J=1.6 Hz, 1H).

Compound 98E: LC-MS (ESI) m/z: 358 [M+H]⁺; ¹H-NMR (CDCl₃, 400 MHz): δ (ppm) 4.54 (d, J=4.8 Hz, 2H), 4.84 (s, 1H), 6.63 (dd, J=6.8, 2.0 Hz, 2H), 7.18 (dd, J=6.8, 2.0 Hz, 2H), 7.77 (d, J=1.6 Hz, 1H), 7.90 (t, J=1.6 Hz, 1H), 8.01 (d, J=1.6 Hz, 1H). Compound 98: LC-MS (ESI) m/z: 511 [M+H]⁺; ¹H-NMR (CD₃OD, 400 MHz): δ (ppm) 4.08 (d, J=10.8 Hz, 1H), 4.25 (d, J=10.8 Hz, 1H), 7.26 (d, J=8.8 Hz, 2H), 7.35-7.41 (m, 4H), 7.51 (s, 1H), 7.62-7.66 (m, 3H), 7.73 (s, 1H).

### Example 99

### Synthesis of 3-(1,3-bis(4-chlorophenyl)-4-hydroxy-5-methyl-2-oxoimidazolidin-4-yl)benzonitrile, 3-((5S)-1,3-bis(4-chlorophenyl)-4-hydroxy-5-methyl-2-oxoimidazolidin-4-yl)benzonitrile, and 3-((5R)-1,3-bis(4-chlorophenyl)-4-hydroxy-5-methyl-2-oxoimidazolidin-4-yl)benzonitrile

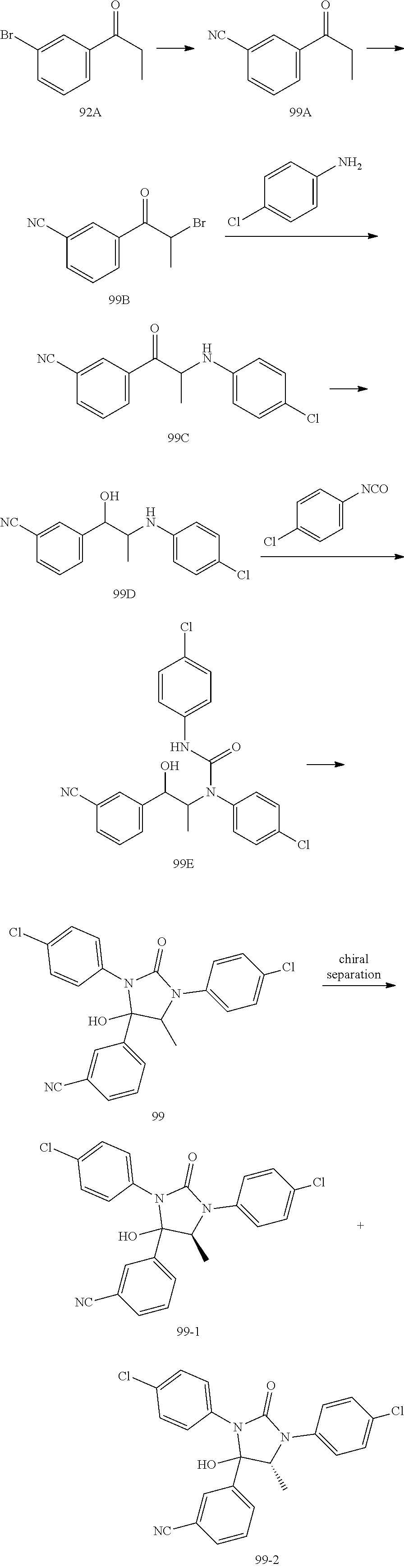

Compounds 99A, 99B, 99C, 99D, 99E, and 99 were synthesized by employing the procedures described for Compounds 95B, 13B, 1B, 14C, 1, and 14 using Compounds 92A, using DMF as solvent, 99A, 99B, 4-chloroaniline, 99C, 99D, 1-chloro-4-isocyanatobenzene, and 99E in lieu of Compounds 95A, using NMP as solvent, 13A, 1A, 4-bromoaniline, 14B, 1B, 1-bromo-4-isocyanatobenzene, and 14D. Compound 99A: LC-MS (ESI) m/z: 160 [M+1]⁺. Compound 99B: LC-MS (ESI) m/z: 238 [M+1]⁺.

Compound 99C: LC-MS (ESI) m/z: 285 [M+1]⁺; ¹H-NMR (CDCl₃, 400 MHz): δ (ppm) 1.50 (d, J=6.8 Hz, 3H), 5.03 (q, J=6.8 Hz, 1H), 6.61-6.63 (m, 2H), 7.13-7.15 (m, 2H), 7.67 (t, J=8.0 Hz, 1H), 7.89-7.91 (m, 1H), 8.20-8.23 (m, 1H), 8.28-8.29 (m, 1H). Compound 99D: LC-MS (ESI) m/z: 287 [M+1]⁺. Compound 99E: LC-MS (ESI) m/z: 440 [M+1]⁺. Compound 99: LC-MS (ESI) m/z: 438 [M+H]⁺; ¹H-NMR (DMSO-d₆, 400 MHz): δ (ppm) 0.64-1.08 (m, 3H), 4.40-4.57 (m, 1H), 7.27-7.32 (m, 2H), 7.41-7.48 (m, 5H), 7.51-7.78 (m, 3H), 7.88-7.97 (m, 1H), 8.07 (s, 1H).

Compound 99 was separated with chiral HPLC to give Compound 99-1 and Compound 99-2. Compound 99-1: LC-MS (ESI) m/z: 438 [M+H]⁺; ¹H-NMR (CD₃OD, 400 MHz): δ (ppm) 0.75-1.20 (m, 3H), 4.42-4.56 (m, 1H), 7.20-7.25 (m, 2H), 7.39-7.45 (m, 5H), 7.50-7.77 (m, 3H), 7.93-7.96 (m, 1H), 8.04 (s, 1H). Chiral separation condition: MeOH contained 0.2% Methanol Ammonia; OD-H (4.6*250 mm, 5 μm); retention time: 1.94 minutes.

Compound 99-2: LC-MS (ESI) m/z: 438 [M+H]⁺; ¹H-NMR (CD₃OD, 400 MHz): δ (ppm) 0.75-1.20 (m, 3H), 4.43-4.55 (m, 1H), 7.20-7.26 (m, 2H), 7.39-7.45 (m, 5H), 7.50-7.78 (m, 3H), 7.93-7.95 (m, 1H), 8.04 (s, 1H). Chiral separation condition: MeOH contained 0.2% Methanol Ammonia; OD-H (4.6*250 mm, 5 μm); retention time: 6.73 minutes.

### Example 100

### Synthesis of 1,3-bis(4-bromophenyl)-4-hydroxy-5-propyl-4-(3-(trifluoromethyl)phenyl)imidazolidin-2-one, (5S)-1,3-bis(4-bromophenyl)-4-hydroxy-5-propyl-4-(3-(trifluoromethyl)phenyl)imidazolidin-2-one, and (5R)-1,3-bis(4-bromophenyl)-4-hydroxy-5-propyl-4-(3-(trifluoromethyl)phenyl)imidazolidin-2-one

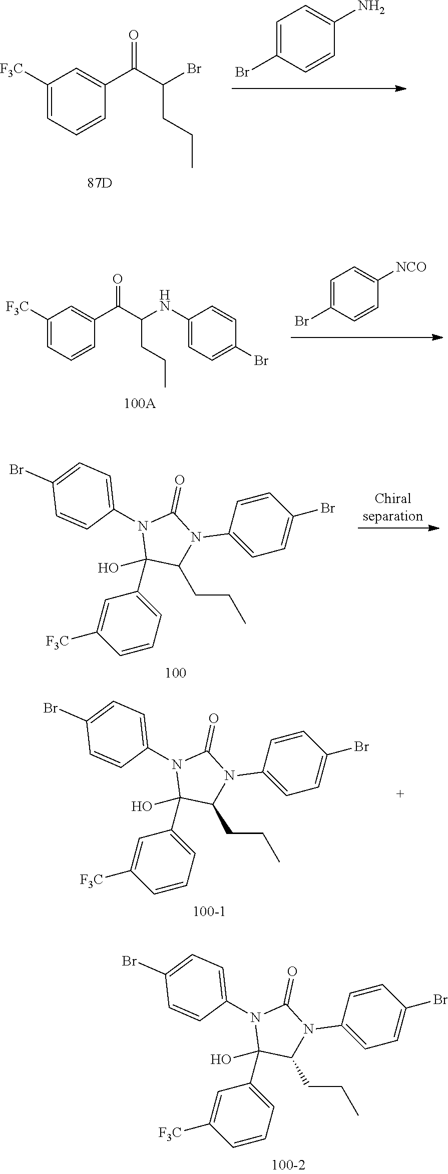

Compounds 100A, and 100 were synthesized by employing the procedures described for Compounds 1B and 1 using Compounds 87D, heating at 60° C. and 100A in lieu of Compounds 1A, stirred at room temperature, and 1B. Compound 100A: LC-MS (ESI) m/z: 400 [M+H]⁺.

Compound 100 was separated with chiral-HPLC to give Compound 100-1 and Compound 100-2. Compound 100-1: LC-MS (ESI) m/z: 597 [M+H]⁺; ¹H-NMR (CDCl₃, 400 MHz): δ (ppm) 0.73 (t, J=7.2 Hz, 3H), 0.84-0.91 (m, 1H), 1.14-1.22 (m, 1H), 1.70-1.84 (m, 2H), 3.59 (s, 1H), 4.22-4.25 (m, 1H), 7.18 (d, J=9.2 Hz, 2H), 7.22 (d, J=8.8 Hz, 2H), 7.41-7.58 (m, 6H), 7.69 (d, J=7.6 Hz, 1H), 7.87 (s, 1H). Chiral separation condition: MeOH contained 0.2% Methanol Ammonia; RegisCell (4.6*250 mm, 5 μm); retention time: 2.66 minutes (85%), 5.58 minutes (15%). Compound 100-1: LC-MS (ESI) m/z: 597 [M+H]⁺; ¹H-NMR (CDCl₃, 400 MHz): δ (ppm) 0.73 (t, J=7.2 Hz, 3H), 0.93-0.99 (m, 1H), 1.17-1.23 (m, 1H), 1.68-1.84 (m, 2H), 3.79 (s, 1H), 4.21-4.25 (m, 1H), 7.15 (d, J=9.2 Hz, 2H), 7.21-7.26 (m, 4H), 7.44 (t, J=7.6 Hz, 1H), 7.51-7.57 (m, 3H), 7.68 (d, J=8.0 Hz, 1H), 7.86 (s, 1H). Chiral separation condition: MeOH contained 0.2% Methanol Ammonia; RegisCell (4.6*250 mm, 5 μm); retention time: 4.24 minutes (16%), 7.87 minutes (82%).

### Example 101

### Synthesis of 3-(1,3-bis(4-chlorophenyl)-4-hydroxy-2-oxo-5-propylimidazolidin-4-yl)benzonitrile, 3-((5S)-1,3-bis(4-chlorophenyl)-4-hydroxy-2-oxo-5-propylimidazolidin-4-yl)benzonitrile, and 3-((5R)-1,3-bis(4-chlorophenyl)-4-hydroxy-2-oxo-5-propylimidazolidin-4-yl)benzonitrile

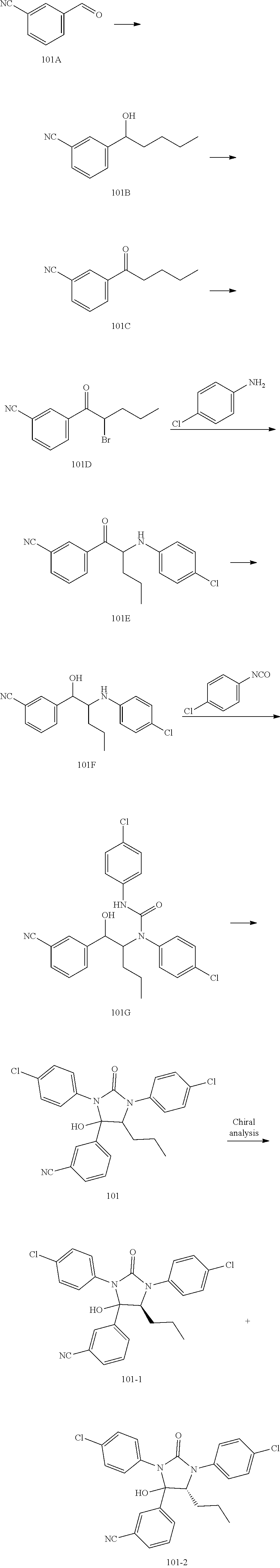

To a solution of Compound 101A (8.5 g, 64.9 mmol) in toluene (300 mL) at room temperature under nitrogen was dropped a solution of n-butylmagnesium chloride in THF (2 M, 32.5 mL, 65 mmol). After stirred for 12 hours, the mixture was quenched with 1 N HCl solution (300 mL) and extracted with ethyl acetate (200 mL×2). The combined organic phases was washed with brine (300 mL), dried over anhydrous sodium sulfate, filtered, and concentrated under reduced pressure. The residue was purified with column chromatography on silica gel (petroleum ether in ethyl acetate, 10% v/v) to yield Compound 101B: LC-MS (ESI) m/z: 190 [M+H]; ¹H-NMR (DMSO-d₆, 400 MHz): δ (ppm) 0.84 (t, J=6.8 Hz, 3H), 1.17-1.29 (m, 4H), 1.55-1.59 (m, 2H), 4.57-4.58 (m, 1H), 5.34 (d, J=4.4 Hz, 1H), 7.51-7.55 (m, 1H), 7.65-7.70 (m, 2H), 7.73-7.74 (m, 1H).

To a solution of Compound 101B (3.4 g, 17.9 mmol) in dichloromethane (100 mL) was added Dess-Martin periodinane (7.58 g, 17.9 mmol). After stirred at room temperature for 12 hours, the mixture was washed with saturated NaHSO₃ solution (50 mL) and brine (100 mL), dried over anhydrous sodium sulfate, filtered, and concentrated under reduced pressure. The residue was purified with column chromatography on silica gel (petroleum ether in ethyl acetate, 5% v/v) to give Compound 101C: LC-MS (ESI) m/z: 188 [M+H]; ¹H-NMR (CDCl₃, 400 MHz): δ (ppm) 0.96 (t, J=7.6 Hz, 3H), 1.39-1.45 (m, 2H), 1.69-1.75 (m, 2H), 2.97 (t, J=7.2 Hz, 2H), 7.58-7.62 (m, 1H), 7.82-7.84 (m, 1H), 8.16-8.19 (m, 1H), 8.23-8.24 (m, 1H).

Compounds 101D, 101E, 101F, 101G, and 101 were synthesized by employing the procedures described for Compounds 13B, 1B, 14C, 1, and 14 using Compounds 101C, 101D, 4-chloroaniline, 101E, 101F, 1-chloro-4-isocyanatobenzene, and 101G in lieu of Compounds 13A, 1A, 4-bromoaniline, 14B, 1B, 1-bromo-4-isocyanatobenzene, and 14D. Compound 101D: LC-MS (ESI) m/z: 266 [M+H]; ¹H-NMR (CDCl₃, 400 MHz): δ (ppm) 1.00 (t, J=7.2 Hz, 3H), 1.26-1.56 (m, 2H), 2.12-2.21 (m, 2H), 5.03-5.07 (m, 1H), 7.62-7.66 (m, 1H), 7.85-7.88 (m, 1H), 8.22-8.25 (m, 1H), 8.29-8.30 (m, 1H). Compound 101E: LC-MS (ESI) m/z: 313 [M+H]⁺; ¹H-NMR (CDCl₃, 400 MHz): δ (ppm) 0.91 (t, J=7.2 Hz, 3H), 1.35-1.48 (m, 2H), 1.61-1.69 (m, 1H), 1.89-1.96 (m, 1H), 4.54 (d, J=8.4 Hz, 1H), 4.92-4.97 (m, 1H), 6.56-6.60 (m, 2H), 7.09-7.13 (m, 2H), 7.64-7.68 (m, 1H), 7.87-7.90 (m, 1H), 8.18-8.21 (m, 1H), 8.26-8.27 (m, 1H). Compound 101F: LC-MS (ESI) m/z: 315 [M+H]⁺. Compound 101G: LC-MS (ESI) m/z: 468 [M+H]⁺. Compound 101: LC-MS (ESI) m/z: 466 [M+H]⁺; (Acetone-d₆, 500 MHz): δ (ppm) 0.51, 0.68 (t, J=7.5 Hz, 3H), 0.71-1.01 (m, 1H), 1.22-1.26 (m, 1H), 1.71-1.95 (m, 2H), 4.50-4.61 (m, 1H), 6.72 (s, 1H), 7.17-7.25 (m, 2H), 7.25-7.46 (m, 4H), 7.55-7.59 (m, 4H), 8.12-8.22 (m, 2H).

Compound 101 was separated with chiral-HPLC to give Compound 101-1 and Compound 101-2. Compound 101-1: LC-MS (ESI) m/z: 466 [M+H]⁺; ¹H-NMR (Acetone-d₆, 500 MHz): δ (ppm) 0.51, 0.70 (t, J=7.5 Hz, 3H), 0.96-1.00 (m, 1H), 1.23-1.27 (m, 1H), 1.72-1.74 (m, 1H), 1.92-1.95 (m, 1H), 4.50-4.61 (m, 1H), 6.74 (s, 1H), 7.17-7.25 (m, 2H), 7.41-7.46 (m, 5H), 7.56-7.70 (m, 3H), 8.12-8.21 (m, 2H). Chiral separation condition: MeOH contained 0.2% Methanol Ammonia; RegisCell (4.6*250 mm, 5 μm); retention time: 2.39 minutes (94%), 3.79 minutes (6%). Compound 101-2: LC-MS (ESI) m/z: 466 [M+H]⁺; ¹H-NMR (Acetone-d₆, 500 MHz): δ (ppm) 0.51, 0.70 (t, J=7.5 Hz, 3H), 0.88-1.00 (m, 1H), 1.23-1.26 (m, 1H), 1.72-1.73 (m, 1H), 1.92-1.95 (m, 1H), 4.59-4.61 (m, 1H), 6.74 (s, 1H), 7.17-7.25 (m, 2H), 7.41-7.46 (m, 5H), 7.57-7.70 (m, 3H), 8.12-8.21 (m, 2H). Chiral separation condition: MeOH contained 0.2% Methanol Ammonia; RegisCell (4.6*250 mm, 5 μm); retention time: 2.99 minutes (16%), 4.83 minutes (83%).

### Example 102

### Synthesis of 3-(1,3-bis(4-chlorophenyl)-4-hydroxy-2-oxoimidazolidin-4-yl)-5-methylbenzonitrile

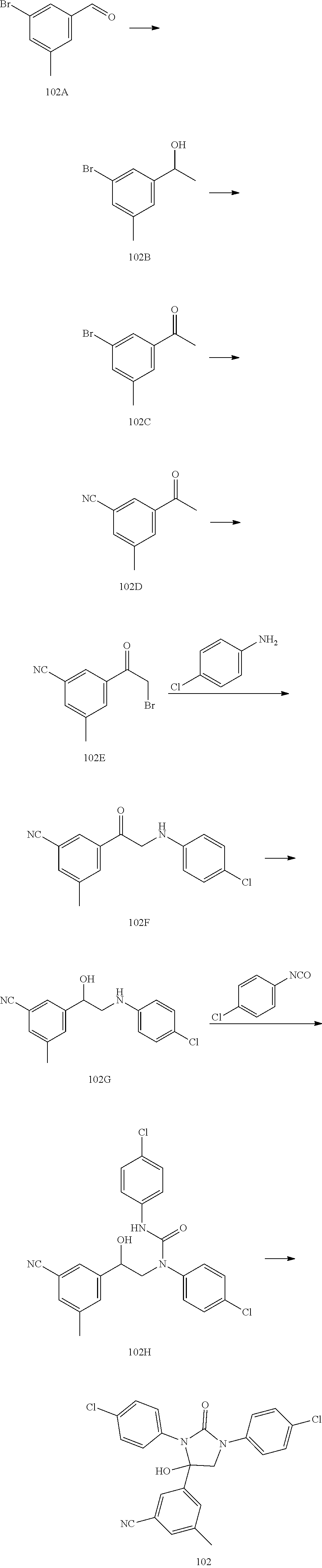

Compounds 102B, 102C, 102D, 102E, 102F, 102G, 102H, and 102 were synthesized by employing the procedures described for Compounds 101B, 101C, 95B, 13B, 1B, 14C, 1, and 14 using Compounds 102A, CH₃MgBr, using THF as solvent and at −78° C., 102B, 102C, 102D, 102E, 4-chloroaniline, heated at 40° C., 102F, 102G, 1-chloro-4-isocyanatobenzene, and 102G in lieu of Compounds 101A, n-butylmagnesium chloride, using toluene as solvent and at room temperature, 101B, 95A, 13A, 1A, 4-bromoaniline, stirred at room temperature, 14B, 1B, 1-bromo-4-isocyanatobenzene, and 14D. Compound 102B: LC-MS (ESI) m/z: non-ionizable compound under routine conditions used; ¹H-NMR (CDCl₃, 400 MHz): δ (ppm) 1.46 (d, J=6.4 Hz, 3H), 2.32 (s, 3H), 4.82 (q, J=6.4 Hz, 1H), 7.09 (s, 1H), 7.23 (s, 1H), 7.31 (s, 1H). Compound 102C: LC-MS (ESI) m/z: non-ionizable compound under routine conditions used; ¹H-NMR (CDCl₃, 400 MHz): δ (ppm) 2.32 (s, 3H), 2.50 (s, 3H), 7.45 (s, 1H), 7.60 (s, 1H), 7.80 (s, 1H). Compound 102D: LC-MS (ESI) m/z: non-ionizable compound under routine conditions used; ¹H-NMR (CDCl₃, 400 MHz): δ (ppm) 2.47 (s, 3H), 2.62 (s, 3H), 7.65 (s, 1H), 7.98 (s, 1H), 8.03 (s, 1H). Compound 102E: LC-MS (ESI) m/z: non-ionizable compound under routine conditions used; ¹H-NMR (CDCl₃, 400 MHz): δ (ppm) 2.42 (s, 3H), 4.33 (s, 2H), 7.62 (s, 1H), 7.94 (s, 1H), 7.99 (s, 1H). Compound 102F: LC-MS (ESI) m/z: 285 [M+H]⁺. Compound 102G: LC-MS (ESI) m/z: 287 [M+H]⁺. Compound 102H): LC-MS (ESI) m/z: 440 [M+H]⁺. Compound 102: LC-MS (ESI) m/z: 438 [M+H]⁺; ¹H-NMR: (DMSO-d₆, 400 MHz): δ (ppm) 2.29 (s, 3H), 4.03 (d, J=10.8 Hz, 1H), 4.17 (d, J=10.4 Hz, 1H), 7.28 (d, J=9.2 Hz, 2H), 7.37 (d, J=8.8 Hz, 2H), 7.42 (d, J=9.2 Hz, 2H), 7.56 (s, 1H), 7.69 (d, J=9.2 Hz, 2H), 7.82 (d, J=20.8 Hz, 2H), 7.90 (s, 1H).

### Example 103

### Synthesis of 1,3-bis(4-chlorophenyl)-4-hydroxy-5-methyl-4-(3-(prop-1-yn-1-yl)phenyl)imidazolidin-2-one, (5S)-1,3-bis(4-chlorophenyl)-4-hydroxy-5-methyl-4-(3-(prop-1-yn-1-yl)phenyl)imidazolidin-2-one, and (5R)-1,3-bis(4-chlorophenyl)-4-hydroxy-5-methyl-4-(3-(prop-1-yn-1-yl)phenyl)imidazolidin-2-one

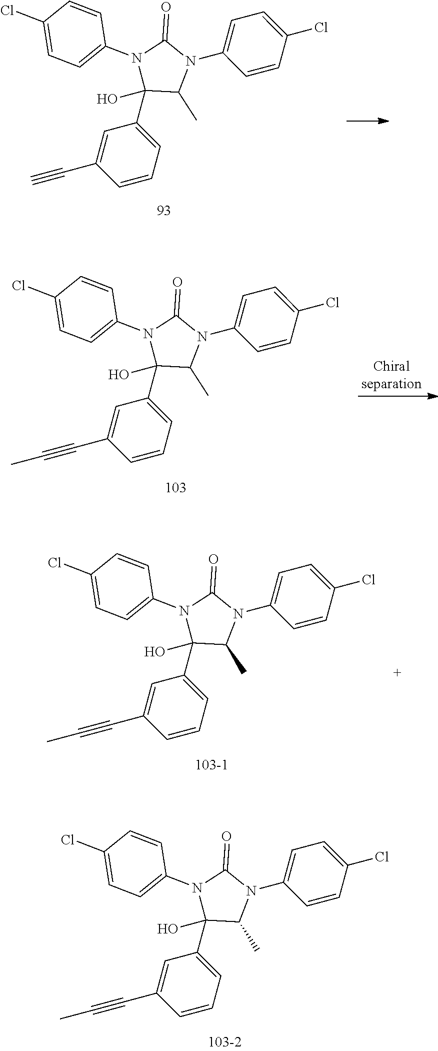 [SMILES: C#Cc1cccc(C2(O)C(C)N(c3ccc(Cl)cc3)C(=O)N2c2ccc(Cl)cc2)c1 ; CC#Cc1cccc(C2(O)C(C)N(c3ccc(Cl)cc3)C(=O)N2c2ccc(Cl)cc2)c1 ; CC#Cc1cccc(C2(O)[C@H](C)N(c3ccc(Cl)cc3)C(=O)N2c2ccc(Cl)cc2)c1 ; CC#Cc1cccc(C2(O)[C@@H](C)N(c3ccc(Cl)cc3)C(=O)N2c2ccc(Cl)cc2)c1]

To a solution of diisopropylamine (103 mg, 1.02 mmol) in dry THF (10 mL) was added a solution of n-BuLi in n-hexane (2.5 N, 1 mmol, 0.4 mL) at −60° C. under nitrogen. After the mixture was stirred at −60° C. for 0.5 hour, a solution of Compound 93 (150 mg, 0.34 mmol) in dry THF (1 mL) was slowly added. The resulting mixture was stirred at −60° C. under nitrogen for 0.5 hour and iodomethane (54 mg, 0.38 mmol) was added. The mixture was stirred at −60° C. for 0.5 hour, quenched with saturated aqueous NH₄Cl solution (5 mL), and extracted with ethyl acetate (20 mL×2). The combined organic layers was washed with brine (20 mL), dried over anhydrous sodium sulfate, filtered, and concentrated under reduced pressure. The residue was purified with preparative HPLC to give Compound 103: LC-MS (ESI) m/z: 451 [M+H]⁺; ¹H-NMR (CD₃OD, 400 MHz): δ (ppm) 0.94, 1.11 (d, J 6.4 Hz, 3H), 1.95 (s, 3H), 4.30-4.43 (m, 1H), 7.12-7.20 (m, 4H), 7.33-7.56 (m, 8H).

Compound 103 was separated with chiral HPLC to give Compound 103-1 and Compound 103-2. Compound 103-1: LC-MS (ESI) m/z: 451 [M+H]⁺; ¹H-NMR (CD₃OD, 400 MHz): δ (ppm) 0.73, 1.17 (d, J=6.4 Hz, 3H), 2.00 (s, 3H), 4.37-4.47 (m, 1H), 7.17-7.27 (m, 4H), 7.37-7.63 (m, 8H); Chiral separation condition: MeOH contained 0.2% Methanol Ammonia; Regiscell (4.6*250 mm, 5 μm); retention time: 3.18 minutes (81%), 5.21 minutes (19%). Compound 103-2: LC-MS (ESI) m/z: 451 [M+H]⁺; ¹H-NMR (CD₃OD, 400 MHz): δ (ppm) 0.61, 1.06 (d, J 6.4 Hz, 3H), 1.83 (s, 3H), 4.22-4.38 (m, 1H), 7.06-7.15 (m, 4H), 7.28-7.55 (m, 8H); Chiral separation condition: MeOH contained 0.2% Methanol Ammonia; Regiscell (4.6*250 mm, 5 μm); retention time: 4.55 minutes (20%), 8.18 minutes (80%).

### Example 104

### Synthesis of 3-(1,3-bis(4-ethylphenyl)-4-hydroxy-5-methyl-2-oxoimidazolidin-4-yl)benzonitrile, 3-((5S)-1,3-bis(4-ethylphenyl)-4-hydroxy-5-methyl-2-oxoimidazolidin-4-yl)benzonitrile, and 3-((5R)-1,3-bis(4-ethylphenyl)-4-hydroxy-5-methyl-2-oxoimidazolidin-4-yl)benzonitrile

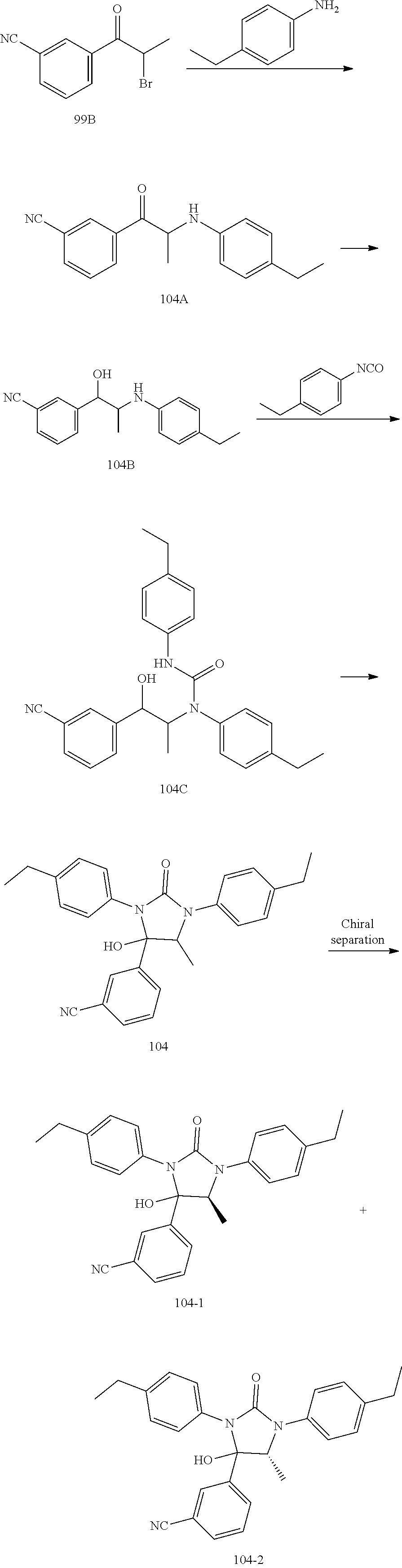

Compounds 104A, 104B, 104C, and 104 were synthesized by employing the procedures described for Compounds 1B, 14C, 1, and 14 using Compounds 99B, 4-ethylaniline, 104A, 104B, 1-ethyl-4-isocyanatobenzene, and 104C in lieu of Compounds 1A, 4-bromoaniline, 14B, 1B, 1-bromo-4-isocyanatobenzene, and 14D. Compound 104A: LC-MS (ESI) m/z: 279 [M+H]⁺. Compound 104B: LC-MS (ESI) m/z: 281 [M+H]⁺. Compound 104C: LC-MS (ESI) m/z: 428 [M+H]⁺. Compound 104: LC-MS: (ESI) m/z: 426 [M+H]⁺. ¹H-NMR (CDCl₃, 400 MHz): δ (ppm) 1.15 (t, J=7.5 Hz, 3H), 1.22-1.26 (m, 6H), 2.51-2.56 (m, 2H), 2.62-2.68 (m, 2H), 3.40 (s, 1H), 4.17-4.22 (m, 1H), 7.01-7.03 (m, 2H), 7.19-7.22 (m, 4H), 7.26-7.28 (m, 2H), 7.44 (t, J=8.0 Hz, 1H), 7.58 (d, J=7.2 Hz, 1H), 7.78 (d, J=8.0 Hz, 1H), 7.95-7.89 (m, 1H).

Compound 104 was separated with chiral HPLC to afford Compound 104-1 and Compound 104-2. Compound 104-1: LC-MS: (ESI) m/z: 426 [M+H]⁺; ¹H-NMR (acetone-d₆, 500 MHz): δ (ppm) 1.12 (t, J=7.6 Hz, 3H), 1.20-1.26 (m, 6H), 2.49-2.53 (m, 2H), 2.62-2.67 (m, 2H), 4.45-4.47 (m, 1H), 6.44 (s, 1H), 7.01-7.03 (m, 2H), 7.22-7.24 (m, 2H), 7.30-7.33 (m, 2H), 7.37-7.39 (m, 2H), 7.57 (t, J=8.0 Hz, 1H), 7.69-7.71 (m, 1H), 8.03-8.06 (m, 1H), 8.11-8.12 (m, 1H). Chiral separation condition: MeOH contained 0.2% Methanol Ammonia; Regiscell (4.6*250 mm, 5 μm); retention time: 3.22 minutes (98%), 6.08 minutes (2%). Compound 104-2: LC-MS: (ESI) m/z: 426 [M+H]⁺; ¹H-NMR (acetone-d₆, 500 MHz): δ (ppm) 1.12 (t, J=8.0 Hz, 3H), 1.20-1.29 (m, 6H), 2.48-2.53 (m, 2H), 2.62-2.66 (m, 2H), 4.44-4.48 (m, 1H), 6.44 (s, 1H), 7.01-7.02 (m, 2H), 7.22-7.24 (m, 2H), 7.30-7.32 (m, 2H), 7.37-7.39 (m, 2H), 7.57 (t, J=7.5 Hz, 1H), 7.69 (d, J=7.5 Hz, 1H), 8.04 (d, J=8.0 Hz, 1H), 8.11 (s, 1H). Chiral separation condition: MeOH contained 0.2% Methanol Ammonia; Regiscell (4.6*250 mm, 5 μm); retention time: 4.22 minutes (1%), 6.20 minutes (98%).

### Example 105

### Synthesis of 3-(4-hydroxy-5-methyl-2-oxo-1,3-di-p-tolylimidazolidin-4-yl)benzonitrile, 3-((5S)-4-hydroxy-5-methyl-2-oxo-1,3-di-p-tolylimidazolidin-4-yl)benzonitrile, and 3-((5R)-4-hydroxy-5-methyl-2-oxo-1,3-di-p-tolylimidazolidin-4-yl)benzonitrile

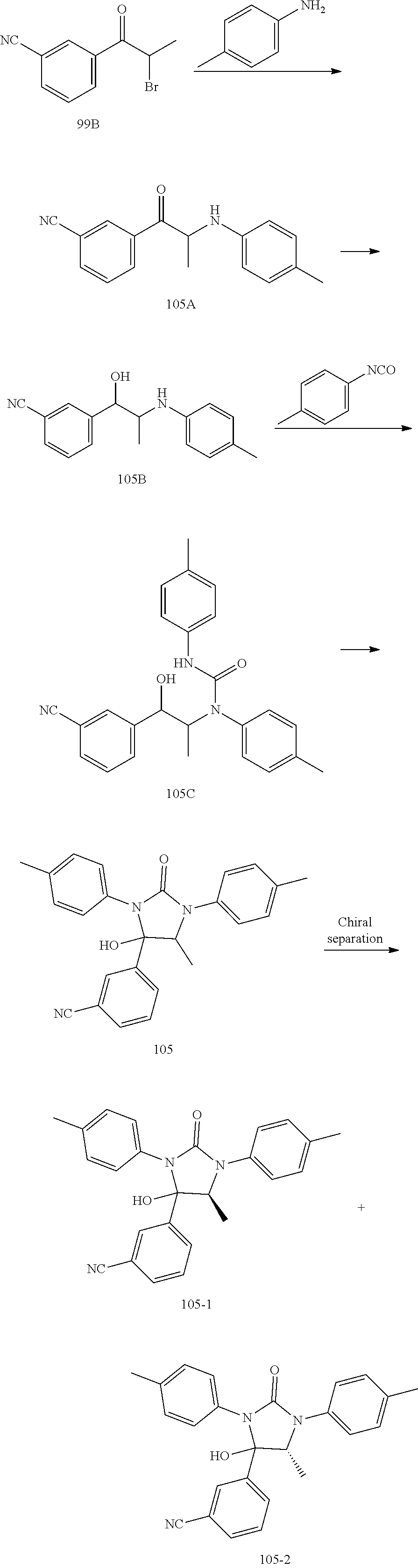

Compounds 105A, 105B, 105C, and 105 were synthesized by employing the procedures described for Compounds 1B, 14C, 1, and 14 using Compounds 99B, 4-methylaniline, 105A, 105B, 1-methyl-4-isocyanatobenzene, and 105C in lieu of Compounds 1A, 4-bromoaniline, 14B, 1B, 1-bromo-4-isocyanatobenzene, and 14D.

Compound 105A: LC-MS (ESI) m/z: 265 [M+H]⁺. Compound 105B: LC-MS (ESI) m/z: 267 [M+H]⁺. Compound 105C: LC-MS (ESI) m/z: 400 [M+H]⁺. Compound 105: LC-MS: (ESI) m/z: 398 [M+H]⁺. ¹H-NMR (CDCl₃, 400 MHz): δ (ppm) 1.22 (d, J=6.8 Hz, 3H), 2.22 (s, 3H), 2.35 (s, 3H), 3.68 (brs, 1H), 4.17-4.18 (m, 1H), 6.97-6.99 (m, 2H), 7.16-7.18 (m, 3H), 7.23-7.25 (m, 3H), 7.44 (t, J=8 Hz, 1H), 7.57 (d, J=7.6 Hz, 1H), 7.77 (d, J=8.4 Hz, 1H), 7.93-7.94 (m, 1H).

Compound 105 was separated with chiral HPLC to afford Compound 105-1 and Compound 105-2. Compound 105-1: LC-MS: (ESI) m/z: 398 [M+H]⁺. ¹H-NMR (acetone-d₆, 500 MHz): δ (ppm) 1.24 (d, J=6.5 Hz, 3H), 2.07 (s, 3H), 2.32 (s, 3H), 4.44-4.47 (m, 1H), 6.43 (s, 1H), 6.96-6.98 (m, 2H), 7.18-7.20 (m, 2H), 7.27-7.29 (m, 2H), 7.34-7.36 (m, 2H), 7.57 (t, J=7.5 Hz, 1H), 7.68-7.70 (m, 1H), 8.04-8.05 (m, 1H), 8.10-8.11 (m, 1H). Chiral separation condition: MeOH contained 0.2% Methanol Ammonia; Regiscell (4.6*250 mm, 5 μm); retention time: 3.14 minutes. Compound 105-2: LC-MS: (ESI) m/z: 398 [M+H]⁺. ¹H-NMR (acetone-d₆, 500 MHz): δ (ppm) 1.24 (d, J=6.5 Hz, 3H), 2.18 (s, 3H), 2.32 (s, 3H), 4.45-4.46 (m, 1H), 6.43 (s, 1H), 6.96-6.98 (m, 2H), 7.18-7.20 (m, 2H), 7.27-7.29 (m, 2H), 7.34-7.36 (m, 2H), 7.57 (t, J=7.5 Hz, 1H), 7.68-7.70 (m, 1H), 8.03-8.05 (m, 1H), 8.10-8.11 (m, 1H). Chiral separation condition: MeOH contained 0.2% Methanol Ammonia; Regiscell (4.6*250 mm, 5 μm); retention time: 6.03 minutes.

### Example 106

### Synthesis of 3-(1,3-bis(4-chlorophenyl)-5-ethyl-4-hydroxy-2-oxoimidazolidin-4-yl)benzonitrile, 3-((5S)-1,3-bis(4-chlorophenyl)-5-ethyl-4-hydroxy-2-oxoimidazolidin-4-yl)benzonitrile, and 3-((5R)-1,3-bis(4-chlorophenyl)-5-ethyl-4-hydroxy-2-oxoimidazolidin-4-yl)benzonitrile

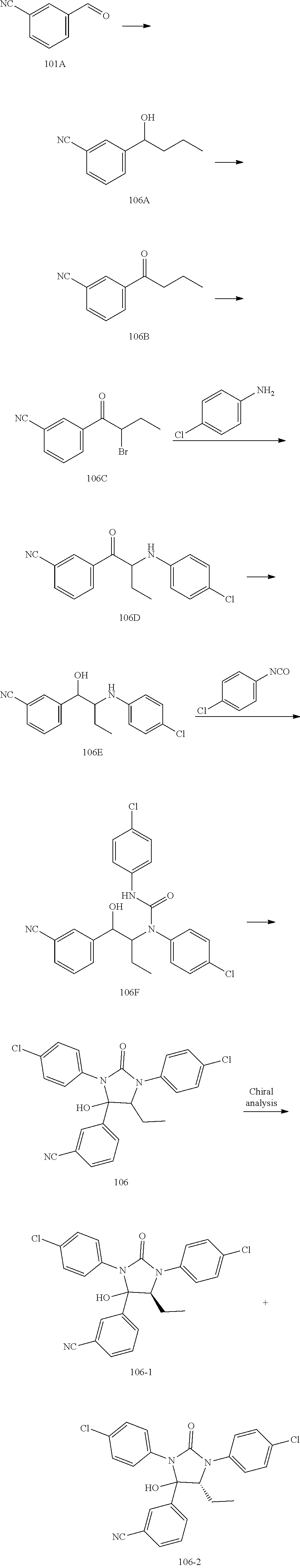

Compounds 106A, 106B, 106C, 106D, 106E, 106F, and 106 were synthesized by employing the procedures described for Compounds 101B, 101C, 13B, 1B, 14C, 1, and 14 using n-propylmagnesium bromide, Compounds 106A, 106B, 106C, 4-chloroaniline, 106D, 106E, 1-chloro-4-isocyanatobenzene, and 106F in lieu of n-butylmagnesium bromide, Compounds 101B, 13A, 1A, 4-bromoaniline, 14B, 1B, 1-bromo-4-isocyanatobenzene, and 14D. Compound 106A: LC-MS (ESI) m/z: 176 [M+H]; ¹H-NMR (CDCl₃, 400 MHz): δ (ppm) 0.94 (t, J=7.6 Hz, 3H), 1.29-1.46 (m, 2H), 1.61-1.79 (m, 2H), 2.13 (br, 1H), 4.71-4.75 (m, 1H), 7.43-7.46 (m, 1H), 7.54-7.59 (m, 2H), 7.65-7.66 (m, 1H). Compound 106B: LC-MS (ESI) m/z: 174 [M+H]; ¹H-NMR (CDCl₃, 400 MHz): δ (ppm) 1.02 (t, J=7.6 Hz, 3H), 1.76-1.81 (m, 2H), 2.96 (t, J=7.2 Hz, 2H), 7.59-7.63 (m, 1H), 7.82-7.84 (m, 1H), 8.17-8.19 (m, 1H), 8.23-8.24 (m, 1H). Compound 106C: LC-MS (ESI) m/z: 252 [M+H]; ¹H-NMR (CDCl₃, 400 MHz): δ (ppm) 1.11 (t, J=7.6 Hz, 3H), 2.12-2.29 (m, 2H), 4.96-5.00 (m, 1H), 7.62-7.66 (m, 1H), 7.86-7.88 (m, 1H), 8.23-8.25 (m, 1H), 8.29-8.30 (m, 1H). Compound 106D: LC-MS (ESI) m/z: 299 [M+H]⁺; ¹H-NMR (CDCl₃, 400 MHz): δ (ppm) 0.91 (t, J=7.2 Hz, 3H), 1.68-1.75 (m, 1H), 2.04-2.11 (m, 1H), 4.62 (s, 1H), 4.95-4.96 (m, 1H), 6.59-6.61 (m, 2H), 7.11-7.14 (m, 2H), 7.64-7.68 (m, 1H), 7.88-7.90 (m, 1H), 8.19-8.21 (m, 1H), 8.26 (s, 1H). Compound 106E: LC-MS (ESI) m/z: 301 [M+H]⁺. Compound 106F: LC-MS (ESI) m/z: 454 [M+H]⁺. Compound 106: LC-MS (ESI) m/z: 452 [M+H]⁺; (Acetone-d₆, 500 MHz): δ (ppm) 0.38, 0.71 (t, J=7.5 Hz, 3H), 1.84-1.97 (m, 2H), 4.52-4.54 (m, 1H), 6.71 (s, 1H), 7.18-7.35 (m, 2H), 7.41-7.71 (m, 8H), 8.12-8.21 (m, 2H).

Compound 106 was separated with chiral HPLC to give Compound 106-1 and Compound 106-2. Compound 106-1: LC-MS (ESI) m/z: 452 [M+H]⁺; ¹H-NMR (Acetone-d₆, 500 MHz): δ (ppm)) 0.71 (t, J=7.5 Hz, 3H), 1.84-1.97 (m, 2H), 4.52-4.55 (m, 1H), 6.72 (s, 1H), 7.18-7.21 (m, 2H), 7.41-7.70 (m, 8H), 8.12-8.21 (m, 2H). Chiral separation condition: MeOH contained 0.2% Methanol Ammonia; RegisCell (4.6*250 mm, 5 μm); retention time: 3.13 minutes (96%), 4.78 minutes (4%). Compound 106-2: LC-MS (ESI) m/z: 452.1 [M+H]⁺; ¹H-NMR (Acetone-d₆, 500 MHz): δ (ppm) 0.31, 0.69 (t, J=7.5 Hz, 3H), 1.82-1.98 (m, 2H), 4.52-4.55 (m, 1H), 6.72 (s, 1H), 7.18-7.25 (m, 2H), 7.41-7.70 (m, 8H), 8.12-8.21 (m, 2H). Chiral separation condition: MeOH contained 0.2% Methanol Ammonia; RegisCell (4.6*250 mm, 5 μm); retention time: 3.94 minutes (7%), 6.01 minutes (93%).

### Example 107

### Synthesis of 4-hydroxy-5-methyl-1,3-di-m-tolyl-4-(3-(trifluoromethoxy)phenyl)imidazolidin-2-one

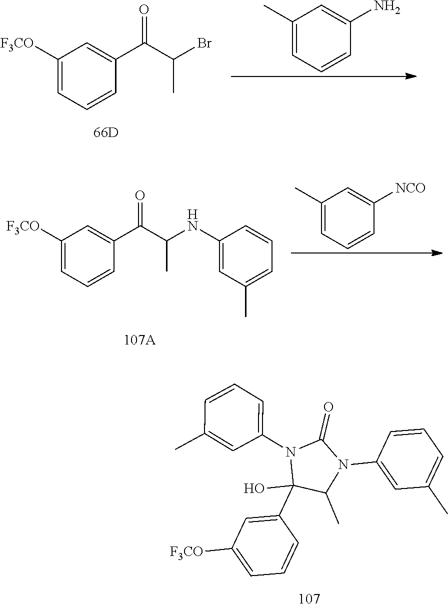

Compounds 107A and 107 were synthesized by employing the procedures described for Compounds 1B and 1 using Compound 66D, m-toluidine, stirred at 60° C., 107A, and 1-isocyanato-3-methylbenzene in lieu of Compounds 1A, 4-bromoaniline, stirred at room temperature, 1B, and 1-bromo-4-isocyanatobenzene. Compound 107A. LC-MS (ESI) m/z: 324 [M+H]⁺; ¹H-NMR (CDCl₃, 400 MHz): δ (ppm) 1.48 (d, J=7.2 Hz, 3H), 2.29 (s, 3H), 4.54 (s, 1H), 5.08 (m, 1H), 6.50 (t, J=6.4 Hz, 2H), 6.58 (d, J=7.6 Hz, 1H), 7.09 (t, J=8 Hz, 1H), 7.48 (d, J=8.0 Hz, 1H), 7.58 (t, J=8.0 Hz, 1H), 7.88 (s, 1H), 7.96 (d, J=7.6 Hz, 1H). Compound 107. LC-MS (ESI) m/z: 457 [M+H]⁺; ¹H-NMR (CDCl₃, 400 MHz): δ (ppm) 0.76, 1.24 (d, J=6.4 Hz, 3H), 2.21, 2.25, 2.36 (s, 6H), 3.30, 3.78 (s, 1H), 4.24, 4.36 (q, J=6.4 Hz, 1H), 6.86-7.17 (m, 6H), 7.24-7.49 (m, 6H).

### Example 108

### Synthesis of 4-(3-chloro-5-ethynylphenyl)-1,3-bis(4-chlorophenyl)-4-hydroxyimidazolidin-2-one

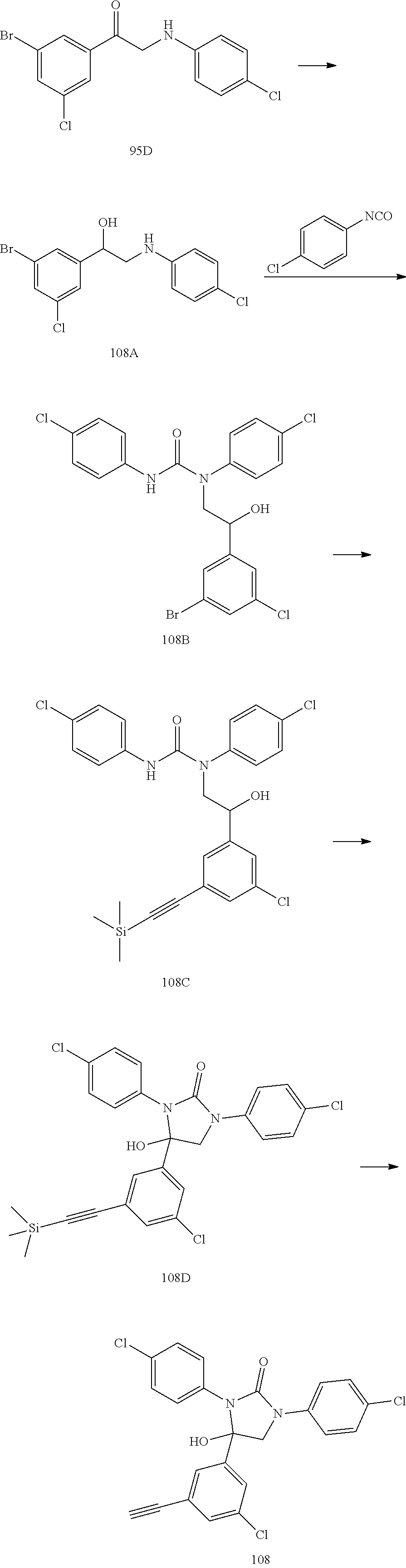

Compounds 108A, 108B, 108C, and 108D were synthesized by employing the procedures described for Compounds 14C, 1, 93B, and 14 using Compounds 95D, 108A, 1-chloro-4-isocyanatobenzene, 108B, and 108C in lieu of Compounds 14B, 1B, 1-bromo-4-isocyanatobenzene, 93A, and 14D. Compound 108A. LC-MS (ESI) m/z: 360 [M+H]⁺; ¹H-NMR (CDCl₃, 400 MHz): δ (ppm) 3.19-3.25 (m, 1H), 3.36-3.41 (m, 1H), 4.85-4.88 (m, 1H), 6.60 (dd, J=2.0, 6.8 Hz, 2H), 7.15 (dd, J=2.0, 6.8 Hz, 2H), 7.34 (s, 1H), 7.46 (dd, J=1.6, 3.6 Hz, 2H). Compound 108B: LC-MS (ESI) m/z: 513 [M+H]⁺; ¹H-NMR (CD₃OD, 400 MHz): (ppm) 3.88-3.92 (m, 2H), 4.89-4.97 (m, 1H), 7.22-7.25 (m, 4H), 7.32-7.41 (m, 5H), 7.45-7.47 (m, 2H). Compound 108C: LC-MS (ESI) m/z: 531 [M+H]⁺; ¹H-NMR (CDCl₃, 400 MHz): C (ppm) 0.25 (t, J=3.6 Hz, 9H), 3.74 (dd, J=2.0, 14.4 Hz, 1H), 4.03-4.14 (m, 1H), 4.77 (d, J=3.6 Hz, 1H), 4.94 (d, J=8.4 Hz, 1H), 6.18 (s, 1H), 7.17-7.20 (m, 2H), 7.29-7.37 (m, 6H), 7.45-7.49 (m, 3H). Compound 108D: LC-MS (ESI) m/z: 529 [M+H]⁺; ¹H-NMR (CD₃OD, 400 MHz): δ (ppm) 0.23 (t, J=3.6 Hz, 9H), 4.07 (d, J=10.4 Hz, 1H), 4.25 (d, J=10.4 Hz, 1H), 7.26 (d, J=8.8 Hz, 2H), 7.34-7.41 (m, 5H), 7.59-7.66 (m, 4H).

Compound 108 was synthesized by employing the procedure described for Compound 93 using Compound 108D in lieu of Compound 93E: LC-MS (ESI) m/z: 457 [M+H]⁺; ¹H-NMR (CD₃OD, 400 MHz): δ (ppm) 3.63 (s, 1H), 4.08 (d, J 10.8 Hz, 1H), 4.25 (d, J 11.2 Hz, 1H), 7.25-7.27 (m, 2H), 7.36-7.41 (m, 5H), 7.63-7.67 (m, 4H).

### Example 109

### Synthesis of 1,3-bis(4-chlorophenyl)-4-hydroxy-4-phenyl-5-(thiophen-2-yl)imidazolidin-2-one

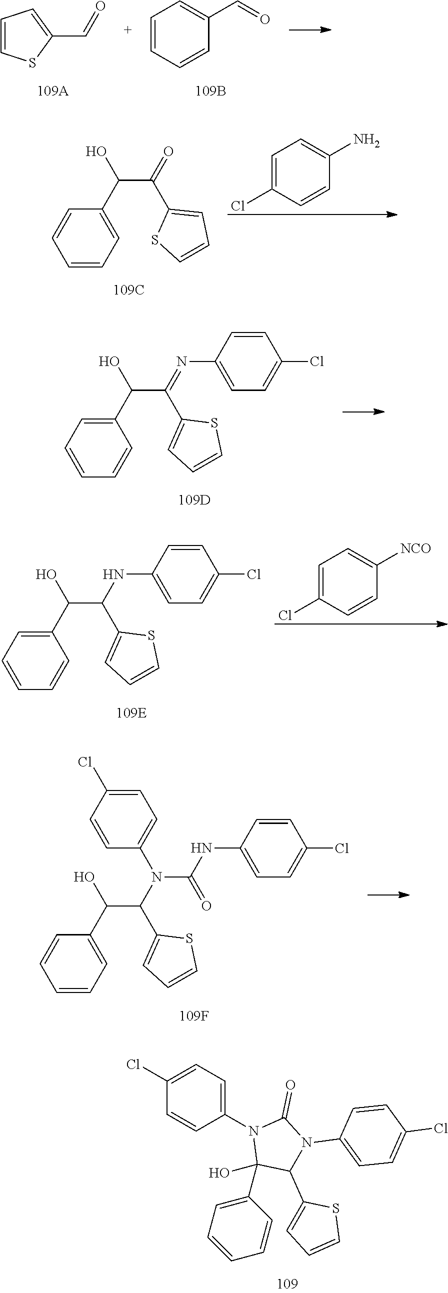

To a mixture of thiophene-2-carbaldehyde 109A (2 g, 17.8 mmol) and sodium cyanide (1.75 mg, 35.6 mmol) in ethanol (40 mL) and H₂O (7 mL) was added benzaldehyde 109B (1.89 g, 17.8 mmol). The mixture was stirred at 100° C. for 3 hours, cooled down to room temperature, diluted with water (100 mL), and extracted with ethyl acetate (60 mL×3). The combined organic layers was washed with water (50 mL×2) and brine (50 mL), dried over anhydrous sodium sulfate, filtered, and concentrated to give a crude product, which was purified with flash column chromatography on silica gel (ethyl acetate in petroleum ether, 20% v/v) to afford Compound 109C: LC-MS (ESI) m/z: 201 [M-OH]⁺; ¹H-NMR (CDCl₃, 400 MHz): δ (ppm) 4.42 (d, J=5.6 Hz, 1H), 5.74 (d, J=5.6 Hz, 1H), 7.04 (t, J=4.4 Hz, 1H), 7.32-7.41 (m, 5H), 7.64 (t, J=4.0 Hz, 2H).

To a mixture of Compound 109C (500 mg, 0.49 mmol) and 4-chloroaniline (235 mg, 1.8 mmol) was added concentrated HCl (0.05 mL, 0.6 mmol). The mixture was stirred at 130° C. for 1 hour, cooled down to room temperature, concentrated. The residue was purified with flash column chromatography on silica gel (ethyl acetate in petroleum ether, 7% v/v) to afford Compound 109D: LC-MS (ESI) m/z: 328 [M+H]⁺; ¹H-NMR (CDCl₃, 400 MHz): δ (ppm) 5.30 (d, J=6.4 Hz, 1H), 6.74 (d, J=6.0 Hz, 1H), 6.56 (d, J=14.8 Hz, 2H), 7.05 (d, J=15.6 Hz, 2H), 7.11 (t, J=4.8 Hz, 1H), 7.26-7.28 (m, 1H), 7.31 (t, J=7.2 Hz, 2H), 7.48 (d, J=6.8 Hz, 2H), 7.65 (dd, J=5.2, 0.8 Hz, 1H), 7.87 (dd, J=3.6, 0.6 Hz, 1H).

Compounds 109E, 109F, and 109 were synthesized by employing the procedures described for Compounds 14C, 1, and 14 using Compounds 109D, 109E, 1-chloro-4-isocyanatobenzene, and 109F in lieu of 14B, 1B, 1-bromo-4-isocyanatobenzene, and 14D. Compound 109E: LC-MS (ESI) m/z: 330 [M+H]⁺; ¹H-NMR (CDCl₃, 400 MHz): δ (ppm) 2.49 (d, J=4.8 Hz, 1H), 4.43 (s, 1H), 4.65 (d, J=4.8 Hz, 1H), 5.26 (t, J=4.8 Hz, 1H), 6.43-6.47 (m, 2H), 6.76 (d, J=3.2 Hz, 1H), 6.92-6.95 (m, 1H), 6.99-7.03 (m, 2H), 7.23-7.28 (m, 4H), 7.29-7.32 (m, 2H). Compound 109F: LC-MS (ESI) m/z: 483 [M+H]⁺; ¹H-NMR: (CDCl₃, 400 MHz): δ (ppm) 2.63 (d, J=3.6 Hz, 1H), 5.23 (d, J=8.8 Hz, 1H), 5.70 (s, 1H), 5.96 (dd, J=8.8, 3.6 Hz, 1H), 6.59-6.62 (m, 2H), 7.04-7.06 (m, 1H), 7.14-7.20 (m, 4H), 7.28-7.30 (m, 3H), 7.32-7.36 (m, 4H), 7.46-7.49 (m, 2H). Compound 109: LC-MS (ESI) m/z: 481 [M+H]⁺; ¹H-NMR (CD₃OD, 400 MHz): δ (ppm) 5.36, 5.62 (s, 1H), 6.86-7.15 (m, 11H), 7.19-7.47 (m, 5H).

### Example 110

### Synthesis of 3-(1-(4-bromophenyl)-3-(4-chlorophenyl)-4-hydroxy-2-oxoimidazolidin-4-yl)-5-chlorobenzonitrile

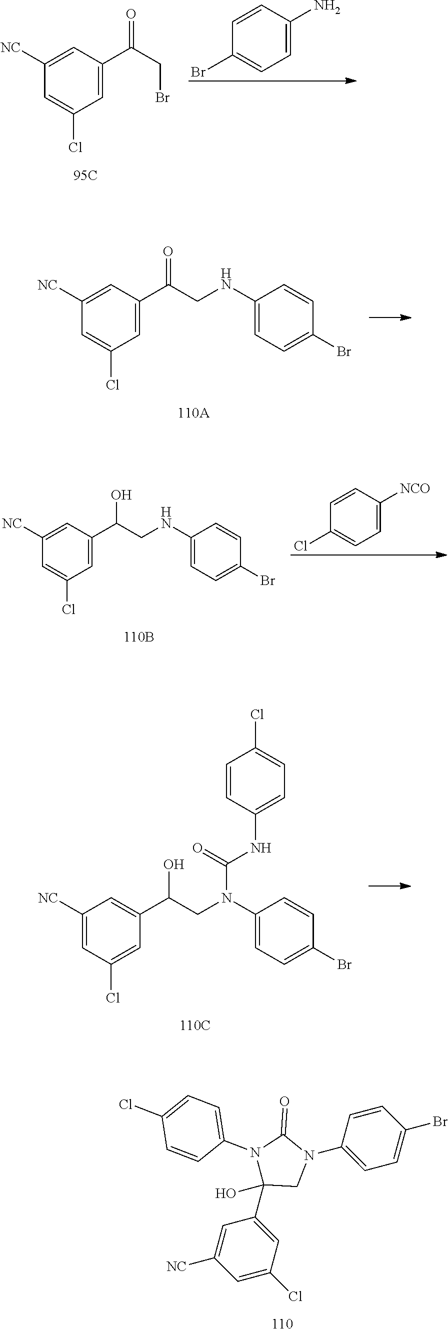

Compounds 110A, 110B, 110C, and 110 were synthesized by employing the procedures described for Compounds 1B, 14C, 1, and 14 using Compounds 95C, 110A, 110B, 1-chloro-4-isocyanatobenzene, and 110C in lieu of 1A, 14B, 1B, 1-bromo-4-isocyanatobenzene, and 14D. Compound 110A: LC-MS (ESI) m/z: 349 [M+H]⁺; ¹H-NMR (DMSO-d₆, 400 MHz): δ (ppm) 4.72-4.73 (m, 2H), 6.15-6.17 (m, 1H), 6.71 (d, J=8.8 Hz, 2H), 7.08 (d, J=8.8 Hz, 2H), 8.33-8.36 (m, 2H), 8.50 (s, 1H). Compound 110B: LC-MS (ESI) m/z: 351 [M+H]⁺. Compound 110C: LC-MS (ESI) m/z: 504 [M+H]⁺; ¹H-NMR: (CDCl₃, 400 MHz): δ (ppm) 3.75-3.79 (m, 1H), 4.04-4.09 (m, 1H), 5.00-5.02 (m, 1H), 5.08-5.09 (m, 1H), 6.13 (s, 1H), 7.11 (d, J=8.8 Hz, 2H), 7.23-7.25 (m, 4H), 7.52 (s, 2H), 7.62 (t, J=7.6 Hz, 3H). Compound 110: LC-MS (ESI) m/z: 502 [M+H]⁺; ¹H-NMR (CDCl₃, 400 MHz): δ (ppm) 3.83 (d, J=10.4 Hz, 1H), 4.01 (d, J=10.4 Hz, 1H), 5.43 (s, 1H), 7.03 (d, J=8.8 Hz, 2H), 7.14 (d, J=8.8 Hz, 2H), 7.25-7.27 (m, 2H), 7.37 (d, J=9.2 Hz, 2H), 7.51 (s, 1H), 7.64 (d, J=9.2 Hz, 2H).

### Example 111

### Synthesis of 4,4′-(4-hydroxy-2-oxo-4-phenylimidazolidine-1,3-diyl)dibenzonitrile

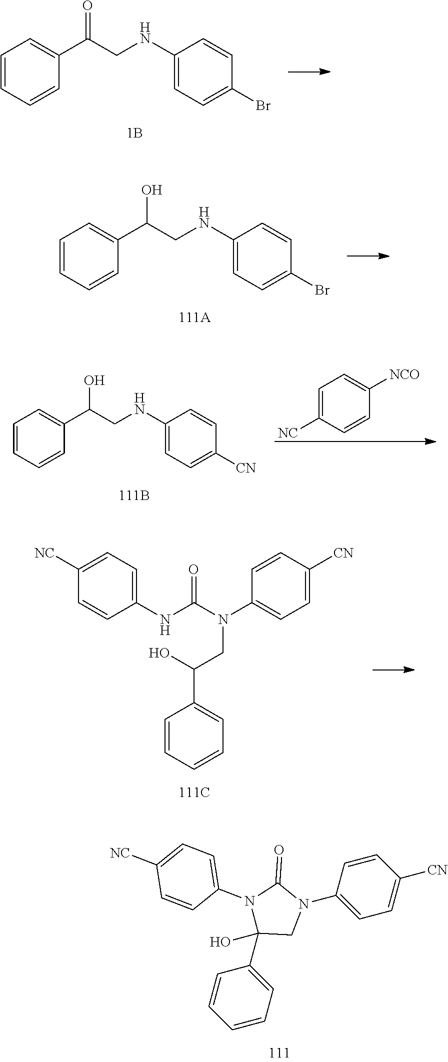

Compounds 111A, 111B, 111C, and 111 were synthesized by employing the procedures described for Compounds 14C, 95B, 1, and 14 using Compounds 1B, 111A with DMF as solvent, 111B and 4-isocyanatobenzonitrile, and 111C in lieu of Compounds 14A, 95A with NMP as solvent, 1B and 1-bromo-4-isocyanatobenzene, and 14D. Compound 111A: LC-MS (ESI) m/z: 292 [M+H]⁺. Compound 111B: LC-MS (ESI) m/z: 239 [M+1]⁺; ¹H-NMR (CDCl₃, 400 MHz): δ (ppm) 2.23-2.24 (m, 1H), 3.33-3.48 (m, 2H), 4.64-4.67 (m, 1H), 4.92-4.96 (m, 1H), 6.59-6.62 (m, 2H), 7.34-7.44 (m, 7H). Compound 111C: LC-MS (ESI) m/z: 365 [M−17]⁺. Compound 111: LC-MS (ESI) m/z: 381 [M+H]⁺; ¹H-NMR (DMSO-d₆, 400 MHz): δ (ppm) 4.13-4.20 (m, 2H), 7.25-7.35 (m, 3H), 7.61-7.69 (m, 6H), 7.84-7.95 (m, 5H).

### Example 112

### Synthesis of 1,3-bis(4-bromophenyl)-4-hydroxy-5-propyl-4-(3-(trifluoromethoxy)phenyl)imidazolidin-2-one, (5S)-1,3-bis(4-bromophenyl)-4-hydroxy-5-propyl-4-(3-(trifluoromethoxy)phenyl)imidazolidin-2-one, and (5R)-1,3-bis(4-bromophenyl)-4-hydroxy-5-propyl-4-(3-(trifluoromethoxy)phenyl)imidazolidin-2-one

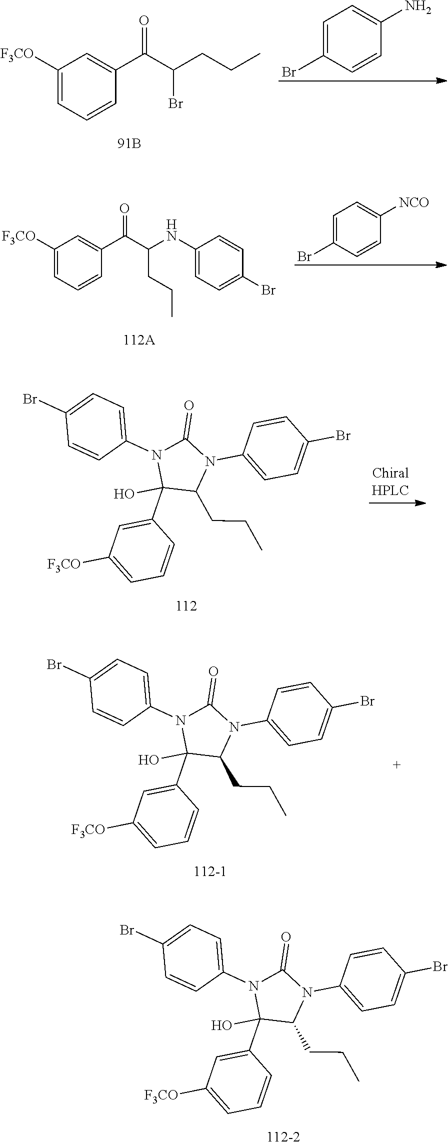

Compounds 112A and 112 were synthesized by employing the procedures described for Compounds 1B and 1 using Compounds 91B, heating at 60° C., and 112A in lieu of Compounds 1A, stirred at room temperature, and 1B. Compound 112A: LC-MS (ESI) m/z: 416 [M+H]⁺.

Compound 112 was separated with chiral HPLC to furnish Compound 112-1 and Compound 112-2. Compound 112-1: LC-MS (ESI) m/z: 613 [M+H]⁺; ¹H-NMR (CDCl₃, 400 MHz): δ (ppm) 0.72, 0.88 (t, J=7.4 Hz, 3H), 0.92-1.35 (m, 2H), 2.02-2.22 (m, 2H), 3.49-4.24 (m, 1H), 5.33-5.37 (m, 1H), 7.15-7.25 (m, 5H), 7.27-7.55 (m, 7H). Chiral separation condition: MeOH contained 0.2% methanol ammonia; RegisCell (4.6*250 mm, 5 μm); retention time: 2.17 minutes (85.4%), 3.86 minutes (14.6%). Compound 112-2: LC-MS (ESI) m/z: 613 [M+H]⁺; ¹H-NMR (CDCl₃, 400 MHz): δ (ppm) 0.72, 0.88 (t, J=7.4 Hz, 3H), 0.92-1.35 (m, 2H), 1.65-2.22 (m, 2H), 3.63-3.65 (m, 0.5H), 4.21-4.24 (m, 1H), 5.34-5.35 (m, 0.5H) 7.13-7.25 (m, 4H), 7.27-7.54 (m, 8H). Chiral separation condition: MeOH contained 0.2% methanol ammonia; RegisCell (4.6*250 mm, 5 m); retention time: 3.08 minutes (66.1%), 5.12 minutes (33.9%).

### Example 113

### Synthesis of 3-(1,3-bis(4-bromophenyl)-4-hydroxy-2-oxoimidazolidin-4-yl)-5-chlorobenzonitrile

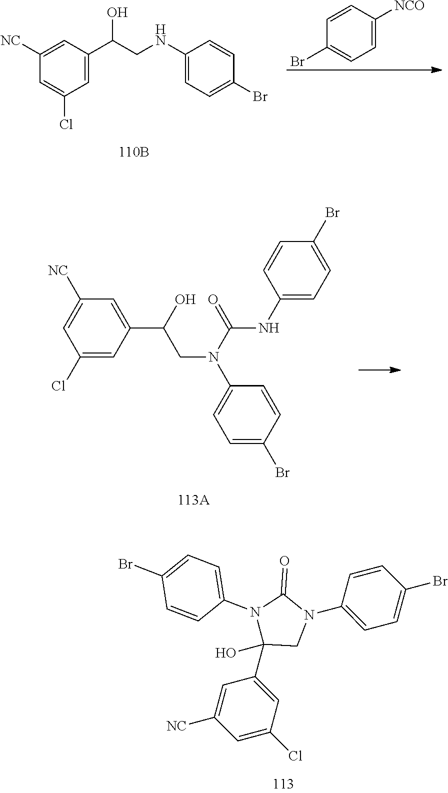

Compounds 113A and 113 were synthesized by employing the procedures described for Compounds 1 and 14 using Compounds 110B and 113A in lieu of Compounds 1B and 14D. Compound 113A: LC-MS (ESI) m/z: 548 [M+H]⁺. Compound 113: LC-MS (ESI) m/z: 546 [M+H]⁺; ¹H-NMR: (CDCl₃, 400 MHz): δ (ppm) 3.83 (d, J=10.4 Hz, 1H), 4.01 (d, J=10.4 Hz, 1H), 5.75 (s, 1H), 7.06 (d, J=9.2 Hz, 2H), 7.15-7.23 (m, 4H), 7.33 (d, J=9.2 Hz, 2H), 7.49 (s, 1H), 7.61-7.66 (m, 2H).

### Example 114

### Synthesis of 4-(3-chlorophenyl)-1,3-bis(4-chlorophenyl)-4-hydroxy-5-propylimidazolidin-2-one, (5S)-4-(3-chlorophenyl)-1,3-bis(4-chlorophenyl)-4-hydroxy-5-propylimidazolidin-2-one, and (5R)-4-(3-chlorophenyl)-1,3-bis(4-chlorophenyl)-4-hydroxy-5-propylimidazolidin-2-one

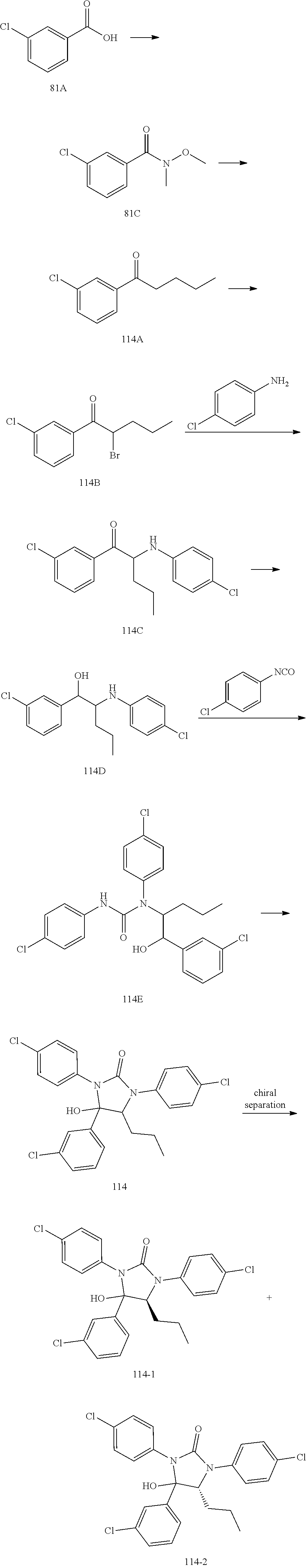

To a solution of 3-chlorobenzoic acid 81A (1.56 g, 10 mmol), N,O-dimethylhydroxylamine hydrochloride (1.2 g, 12.8 mmol), and EDCI (2.5 g, 12.8 mmol) in dichloromethane (40 mL) was dropped triethylamine (3 g, 30 mmol) at 0° C. The mixture was stirred at 25° C. for 16 hours and diluted with dichloromethane (50 mL). The organic layer was washed with water (30 mL×2) and brine (30 mL), dried over anhydrous sodium sulfate, filtered, and concentrated to leave a crude Compound 81C. LC-MS (ESI) m/z: 200 [M+H]⁺; ¹H-NMR (CDCl₃, 400 MHz): δ (ppm) 3.36 (s, 3H), 3.55 (s, 3H), 7.34 (t, J=8.0 Hz, 1H), 7.42-7.44 (m, 1H), 7.55-7.57 (m, 1H), 7.66 (s, 1H).

Compounds 114A, 114B, 114C, 114D, 114E, and 114 were synthesized by employing the procedures described for Compounds 42C, 13B, 1B, 14C, 1, and 14 using n-butylmagnesium chloride, Compounds 81C, 114A, 114B, 4-chloroaniline, 114C, 114D, 1-chloro-4-isocyanatobenzene, and 114E in lieu of EtMgBr, Compounds 42B, 13A, 1A, 4-bromoaniline, 14B, 1B, 1-bromo-4-isocyanatobenzene, and 14D. Compound 114A: LC-MS (ESI) m/z: 197 [M+H]⁺; ¹H-NMR (CDCl₃, 400 MHz): δ (ppm) 0.95 (t, J=7.2 Hz, 3H), 1.39-1.43 (m, 2H), 1.69-1.73 (m, 2H), 2.94 (t, J=7.6 Hz, 2H), 7.40 (t, J=8.0 Hz, 1H), 7.51 (d, J=9.2 Hz, 1H), 7.82 (d, J=9.2 Hz, 1H), 7.92 (s, 1H). Compound 114B: LC-MS (ESI) m/z: 275 [M+H]⁺. Compound 114C: LC-MS (ESI) m/z: 322 [M+H]⁺. Compound 114D: LC-MS (ESI) m/z: 324 [M+H]⁺. Compound 114E: LC-MS (ESI) m/z: 477 [M+H]⁺. Compound 114: LC-MS (ESI) m/z: 475 [M+H]⁺; ¹H-NMR (CDCl₃, 400 MHz): δ (ppm) 0.52-0.75 (m, 3H), 0.95-1.31 (m, 2H), 1.69-1.79 (m, 2H), 3.42, 3.89 (s, 1H), 4.14-4.24 (m, 1H), 7.12-7.15 (m, 2H), 7.25-7.43 (m, 9H), 7.59 (s, 1H).

Compound 114 was separated with chiral-HPLC to afford Compound 114-1 and Compound 114-2. Compound 114-1: LC-MS (ESI) m/z: 475 [M+H]⁺; ¹H-NMR (CDCl₃, 400 MHz): δ (ppm) 0.51-0.75 (m, 3H), 0.85-1.05 (m, 2H), 1.68-2.04 (m, 2H), 3.47, 3.92 (s, 1H), 4.14-4.24 (m, 1H), 7.14-7.20 (m, 2H), 7.25-7.45 (m, 9H), 7.59 (s, 1H). Chiral separation condition: MeOH contained 0.2% methanol ammonia; OD-H (4.6*150 mm, 5 μm); retention time: 1.62 minutes (85.2%), 3.74 minutes (14.9%). Compound 114-2: LC-MS (ESI) m/z: 475 [M+H]⁺; ¹H-NMR (CDCl₃, 400 MHz): δ (ppm) 0.51-0.74 (m, 3H), 0.83-1.05 (m, 2H), 1.69-2.21 (m, 2H), 3.60, 4.08 (s, 1H), 4.14-4.24 (m, 1H), 7.11-7.20 (m, 2H), 7.25-7.45 (m, 9H), 7.58 (s, 1H). Chiral separation condition: MeOH contained 0.2% methanol ammonia; OD-H (4.6*150 mm, 5 μm); retention time: 2.52 minutes (15.6%), 4.99 minutes (84.3%).

### Example 115

### Synthesis of 1,3-bis(4-bromophenyl)-5-cyclopropyl-4-hydroxy-4-phenylimidazolidin-2-one, (5S)-1,3-bis(4-bromophenyl)-5-cyclopropyl-4-hydroxy-4-phenylimidazolidin-2-one, and (5R)-1,3-bis(4-bromophenyl)-5-cyclopropyl-4-hydroxy-4-phenylimidazolidin-2-one

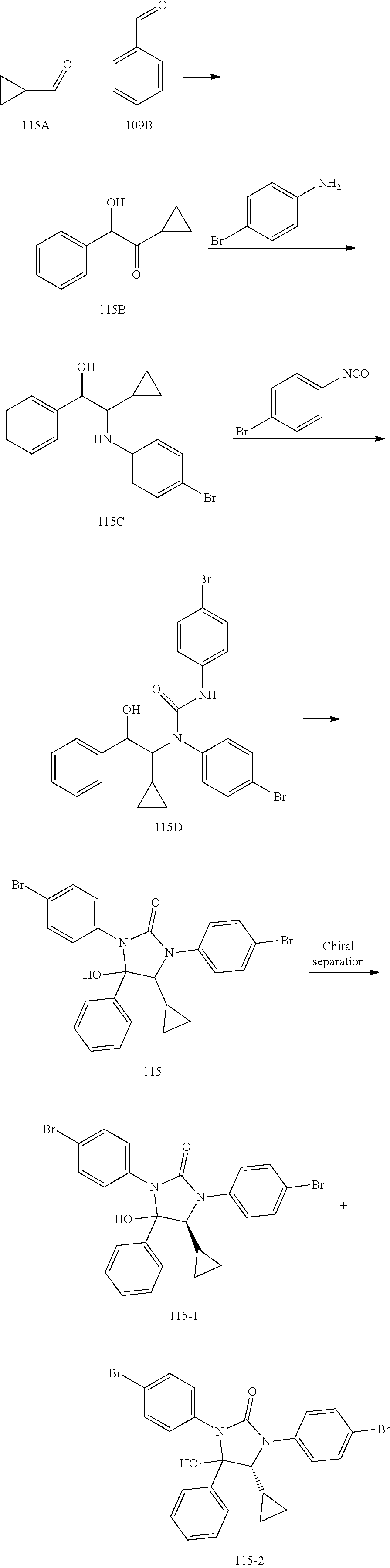

Compound 115B was synthesized by employing the procedure described for Compound 109C using Compound 115A in lieu of Compound 109A: LC-MS (ESI) m/z: 177 [M+H]⁺; ¹H-NMR (CDCl₃, 400 MHz): δ (ppm) 0.78-0.83 (m, 1H), 1.00-1.16 (m, 2H), 1.24-1.28 (m, 1H), 1.85-1.89 (m, 1H), 4.40 (d, J=4.4 Hz, 1H), 5.26 (d, J=4.4 Hz, 1H), 7.34-7.39 (m, 5H).

A solution of Compound 115B (1.0 g, 5.7 mmol) and 4-bromoaniline (1.0 g, 5.7 mmol) in ethanol (10 mL) was stirred at 130° C. for 16 hours in a sealed vial. After the reaction mixture was cooled down to room temperature, to it was added NaBH₄ (0.2 g, 5.7 mmol), stirred at 25° C. for 0.5 hour, and evaporated under reduced pressure. The residue was purified with preparative HPLC to afford Compound 115C: LC-MS (ESI) m/z: 332 [M+H]⁺; ¹H-NMR (CDCl₃, 400 MHz): δ (ppm) −0.06-0.00 (m, 1H), 0.14-0.18 (m, 1H), 0.34-0.40 (m, 2H), 0.82-0.84 (m, 1H), 2.54 (s, 1H), 3.02 (s, 1H), 3.79 (s, 1H), 5.01 (s, 1H), 6.58 (d, J=8.8 Hz, 2H), 7.25 (d, J=8.8 Hz, 2H), 7.30-7.40 (m, 5H).

Compounds 115D and 115 were synthesized by employing the procedures described for Compounds 1 and 14 using Compounds 115C and 115D in lieu of Compounds 1B and 14D. Compound 115D: LC-MS (ESI) m/z: 529 [M+H]⁺; ¹H-NMR (CD₃OD, 400 MHz): δ (ppm) −0.05-−0.02 (m, 1H), 0.20-0.23 (m, 1H), 0.31-0.34 (m, 1H), 0.47-0.49 (m, 1H), 0.99 (s, 1H), 3.66 (d, J=6.8 Hz, 1H), 5.19 (d, J=4.4 Hz, 1H), 7.15-7.37 (m, 10H), 7.45-7.47 (m, 2H), 7.53-7.55 (m, 2H). Compound 115 LC-MS (ESI) m/z: 527 [M+H]⁺; ¹H-NMR (CD₃OD, 400 MHz): δ (ppm) −0.31-0.00 (m, 2H), 0.35-0.47 (m, 2H), 1.00-1.15 (m, 1H), 3.58-3.67 (m, 1H), 7.38-7.56 (m, 9H), 7.67-7.75 (m, 4H).

Compound 115 was separated with chiral HPLC to give Compound 115-1 and Compound 115-2. Compound 115-1 LC-MS (ESI) m/z: 527 [M+H]⁺; ¹H-NMR (CD₃OD, 400 MHz): δ (ppm) −0.29-0.06 (m, 2H), 0.31-0.59 (m, 2H), 1.07-1.20 (m, 1H), 3.56-3.67 (m, 1H), 7.25-7.78 (m, 13H); Chiral separation condition: MeOH contained 0.2% methanol ammonia; OJ-H (4.6*250 mm, 5 μm); retention time: 2.65 minutes (71%), 4.54 minutes (29%). Compound 115-2 LC-MS (ESI) m/z: 527 [M+H]⁺; ¹H-NMR (CD₃OD, 400 MHz): δ (ppm) −0.29-0.01 (m, 2H), 0.31-0.49 (m, 2H), 1.11-1.19 (m, 1H), 3.59-3.68 (m, 1H), 7.39-7.70 (m, 13H); Chiral separation condition: MeOH contained 0.2% methanol ammonia; OJ-H (4.6*250 mm, 5 μm); retention time: 3.24 minutes (71%), 5.61 minutes (29%).

### Example 116

### Synthesis of 3-(5-butyl-1,3-bis(4-chlorophenyl)-4-hydroxy-2-oxoimidazolidin-4-yl)benzonitrile, 3-((5S)-5-butyl-1,3-bis(4-chlorophenyl)-4-hydroxy-2-oxoimidazolidin-4-yl)benzonitrile, and 3-((5R)-5-butyl-1,3-bis(4-chlorophenyl)-4-hydroxy-2-oxoimidazolidin-4-yl)benzonitrile

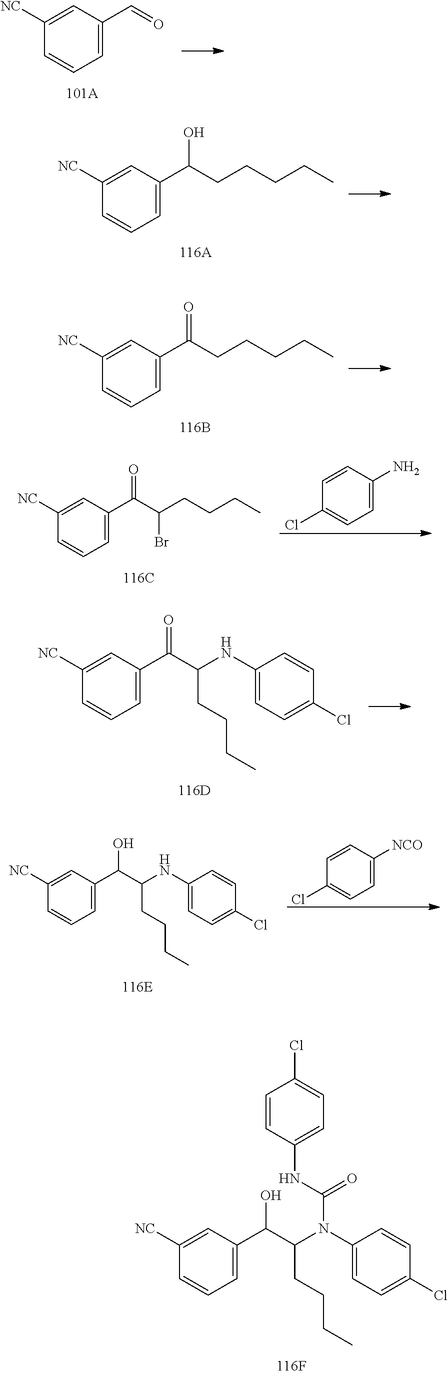

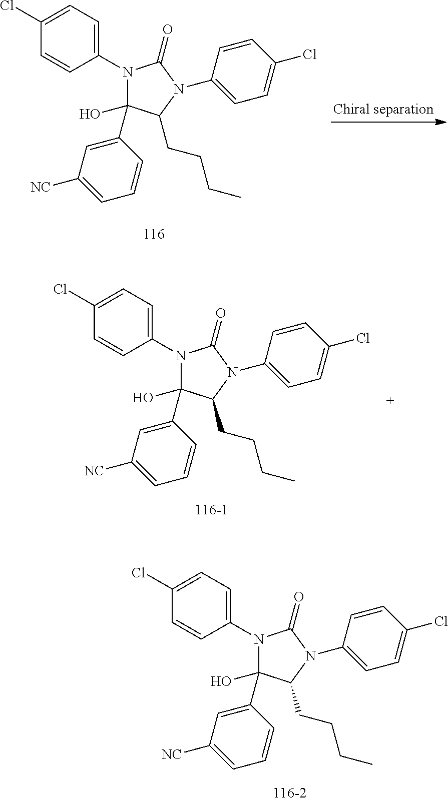 [SMILES: CCCCC1N(c2ccc(Cl)cc2)C(=O)N(c2ccc(Cl)cc2)C1(O)c1cccc(C#N)c1 ; CCCC[C@@H]1N(c2ccc(Cl)cc2)C(=O)N(c2ccc(Cl)cc2)C1(O)c1cccc(C#N)c1 ; CCCC[C@H]1N(c2ccc(Cl)cc2)C(=O)N(c2ccc(Cl)cc2)C1(O)c1cccc(C#N)c1]

Compounds 116A, 116B, 116C, 116D, 116E, 116F, and 116 were synthesized by employing the procedures described for Compounds 101B, 101C, 13B, 1B, 14C, 1, and 14 using n-pentylmagnesium bromide, Compounds 116A, 116B, 116C, 4-chloroaniline, heated at 70° C., 116D, 116E, 1-chloro-4-isocyanatobenzene, and 116F in lieu of n-butylmagnesium bromide, Compounds 101B, 13A, 1A, 4-bromoaniline, stirred at room temperature, 14B, 1B, 1-bromo-4-isocyanatobenzene, and 14D. Compound 116A: LC-MS (ESI) m/z: non-ionizable compound under routine conditions used; ¹H-NMR (CDCl₃, 400 MHz): δ (ppm) 0.87 (t, J=7.2 Hz, 3H), 1.29-1.43 (m, 6H), 1.68-1.75 (m, 2H), 4.70-4.73 (m, 1H), 7.45 (t, J=8.0 Hz, 1H), 7.54-7.59 (m, 2H), 7.65 (s, 1H). Compound 116B: LC-MS (ESI) m/z: 202 [M+H]⁺. Compound 116C: LC-MS (ESI) m/z: non-ionizable compound under routine conditions used; ¹H-NMR (CDCl₃, 400 MHz): δ (ppm) 0.94 (t, J=7.2 Hz, 3H), 1.38-1.55 (m, 4H), 2.10-2.24 (m, 2H), 5.01-5.05 (m, 1H), 7.64 (t, J=8.0 Hz, 1H), 7.85 (d, J=6.8 Hz, 1H), 8.22-8.25 (m, 1H), 8.29 (s, 1H). Compound 116D: LC-MS (ESI) m/z: 327 [M+H]⁺.

Compound 116E: LC-MS (ESI) m/z: 329 [M+H]⁺; ¹H-NMR (CDCl₃, 400 MHz): δ (ppm) 0.79 (t, J=7.2 Hz, 3H), 1.16-1.58 (m, 6H), 3.57-3.64 (m, 2H), 4.94-4.96 (m, 1H), 6.59-6.62 (m, 3H), 7.10 (q, J=8.8 Hz, 3H), 7.47 (t, J=7.6 Hz, 1H), 7.59 (t, J=6.8 Hz, 1H), 7.68 (s, 1H). Compound 116F: LC-MS (ESI) m/z: 482 [M+H]⁺. Compound 116: LC-MS (ESI) m/z: 480 [M+H]⁺; ¹H-NMR (CDCl₃, 400 MHz): δ (ppm) 0.50, 0.72 (t, J=7.2 Hz, 3H), 0.75-1.31 (m, 5H), 1.66-1.80 (m, 1H), 4.11-4.17 (m, 1H), 3.82, 4.62 (s, 1H), 7.09-7.13 (m, 5H), 7.29-7.41 (m, 4H), 7.46-7.92 (m, 3H).

Compound 116 was separated with chiral HPLC to afford Compound 116-1 and Compound 116-2. Compound 116-1: LC-MS (ESI) m/z: 480 [M+H]⁺; ¹H-NMR (CDCl₃, 400 MHz): δ (ppm) 0.49, 0.73 (t, J=7.2 Hz, 3H), 0.86-1.15 (m, 4H), 1.62-2.17 (m, 2H), 4.08-4.18 (m, 1H), 4.27, 4.97 (s, 1H), 7.05-7.24 (m, 6H), 7.29-7.57 (m, 4H), 7.71 (d, J=8.0 Hz, 1H), 7.89 (s, 1H); Chiral separation condition: MeOH contained 0.2% methanol ammonia; OD-H (4.6*150 mm, 5 μm); retention time: 1.81 minutes (85.34%), 4.10 minutes (14.56%). Compound 116-2: LC-MS (ESI) m/z: 480 [M+H]⁺; ¹H-NMR (CDCl₃, 400 MHz): δ (ppm) 0.49, 0.73 (t, J=7.2 Hz, 3H), 0.84-1.15 (m, 4H), 1.59-2.19 (m, 2H), 4.09-4.19 (m, 1H), 4.05, 4.70 (s, 1H), 7.06-7.26 (m, 5H), 7.34-7.56 (m, 5H), 7.72 (d, J=8.0 Hz, 1H), 7.90 (s, 1H); Chiral separation condition: MeOH contained 0.2% methanol ammonia; OD-H (4.6*150 mm, 5 μm); retention time: 2.88 minutes (22.9%), 5.15 minutes (77.1%).

### Example 117

### Synthesis of (5S)-4-(3-chlorophenyl)-1,3-bis(4-chlorophenyl)-4-hydroxy-5-((S)-tetrahydrofuran-2-yl)imidazolidin-2-one, and (5R)-4-(3-chlorophenyl)-1,3-bis(4-chlorophenyl)-4-hydroxy-5-((R)-tetrahydrofuran-2-yl)imidazolidin-2-one

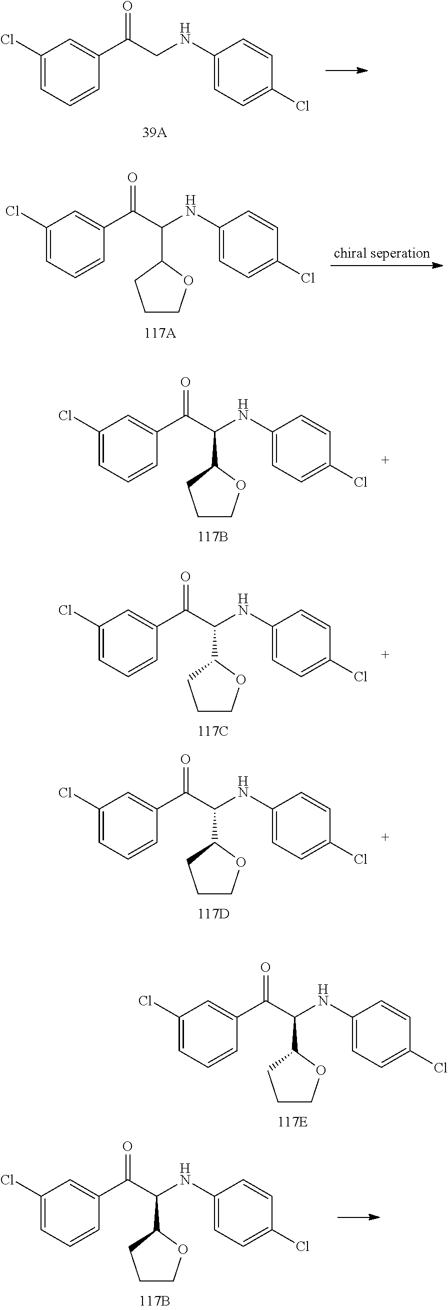

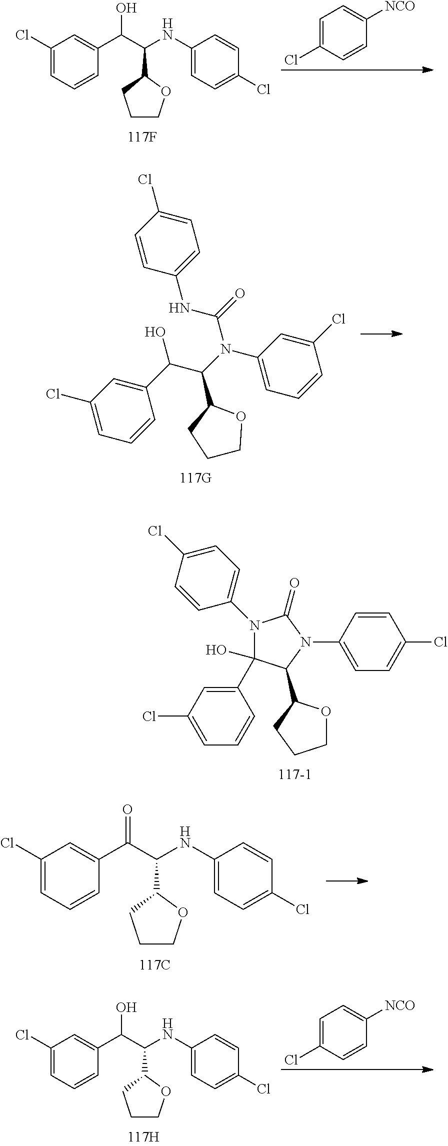

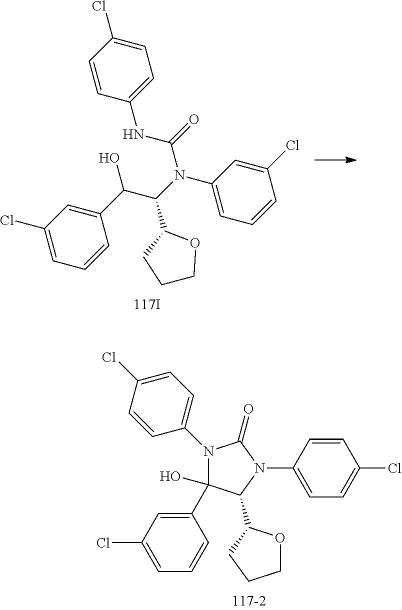 [SMILES: O=C(Nc1ccc(Cl)cc1)N(c1cccc(Cl)c1)[C@@H](C(O)c1cccc(Cl)c1)[C@H]1CCCO1 ; O=C1N(c2ccc(Cl)cc2)[C@H]([C@H]2CCCO2)C(O)(c2cccc(Cl)c2)N1c1ccc(Cl)cc1]

To a solution of Compound 39A (8 g, 0.028 mol) in dry tetrahydrofuran (60 mL) was added CuCl₂ (188 mg, 0.0014 mol), followed by anhydrous tert-butyl hydrogen peroxide solution (5 M in decane, 6.8 mL, 0.034 mol). The mixture was stirred at room temperature for 8 hours, diluted with brine (40 mL), and extracted with ethyl acetate (40 mL×2). The combined organic phases was dried and concentrated under reduced pressure. The residue was purified with preparative HPLC to yield Compound 117A: LC-MS (ESI) m/z: 350 [M+H]⁺; ¹H-NMR (CDCl₃, 400 MHz): δ (ppm) 1.81-2.04 (m, 4H), 3.69-3.80 (m, 2H), 4.28-4.35 (m, 1H), 4.80 (d, J=7.6 Hz, 1H), 4.96-5.10 (m, 1H), 6.60 (d, J=8.4 Hz, 1H), 6.73 (d, J=6.8 Hz, 1H), 7.09-7.12 (m, 2H), 7.42-7.46 (m, 1H), 7.56-7.57 (m, 1H), 7.87-7.91 (m, 1H), 7.97-7.98 (m, 1H).

Compound 117A was separated with chiral HPLC to give Compound 117B, Compound 117C, Compound 117D, and Compound 117E. Compound 117B: LC-MS (ESI) m/z: 350 [M+H]⁺; ¹H-NMR (CDCl₃, 400 MHz): δ (ppm) 1.79-1.92 (m, 4H), 3.71-3.81 (m, 2H), 4.28-4.31 (m, 1H), 4.80 (d, J=7.6 Hz, 1H), 5.06-5.09 (m, 1H), 6.73-6.75 (m, 2H), 7.08-7.11 (m, 2H), 7.44 (t, J=7.6 Hz, 1H), 7.56-7.59 (m, 1H), 7.88 (d, J=7.6 Hz, 1H), 7.97 (d, J=1.6 Hz, 1H). Chiral separation condition: MeOH contained 0.2% Methanol Ammonia); AY-H (250*4.6 mm, 5 μm); retention time: 2.47 minutes. Compound 117C: LC-MS (ESI) m/z: 350 [M+H]⁺; ¹H-NMR (CDCl₃, 400 MHz): δ (ppm) 1.79-1.92 (m, 4H), 3.71-3.81 (m, 2H), 4.29 (m, 1H), 4.80 (s, 1H), 5.07-5.08 (m, 1H), 6.73-6.76 (m, 2H), 7.08-7.11 (m, 2H), 7.44 (t, J=7.6 Hz, 1H), 7.56-7.59 (m, 1H), 7.88 (d, J=7.6 Hz, 1H), 7.97 (t, J=1.6 Hz, 1H). Chiral separation condition: MeOH contained 0.2% Methanol Ammonia); AY-H (250*4.6 mm, 5 μm); retention time: 6.3 minutes. Compound 117D: LC-MS (ESI) m/z: 350 [M+H]⁺; ¹H-NMR (CDCl₃, 400 MHz): δ (ppm) 1.82-1.88 (m, 2H), 1.90-2.07 (m, 2H), 3.65-3.81 (m, 2H), 4.31-4.35 (m, 1H), 4.79 (d, J=8.8 Hz, 1H), 4.96-4.97 (m, 1H), 6.59-6.61 (m, 2H), 7.09-7.11 (m, 2H), 7.43 (t, J=7.6 Hz, 1H), 7.56-7.58 (m, 1H), 7.89 (d, J=7.6 Hz, 1H), 7.97 (d, J=1.6 Hz, 1H). Chiral separation condition: MeOH contained 0.2% Methanol Ammonia); AY-H (250*4.6 mm, 5 μm); retention time: 3.16 minutes. Compound 117E: LC-MS (ESI) m/z: 350 [M+H]⁺; ¹H-NMR (CDCl₃, 400 MHz): δ (ppm) 1.82-1.88 (m, 2H), 1.95-2.02 (m, 2H), 3.65-3.81 (m, 2H), 4.31-4.35 (m, 1H), 4.79 (d, J=8.8 Hz, 1H), 4.96-4.98 (m, 1H), 6.59-6.61 (m, 2H), 7.09-7.11 (m, 2H), 7.43 (t, J=7.6 Hz, 1H), 7.55-7.58 (m, 1H), 7.89 (d, J=7.6 Hz, 1H), 7.97 (d, J=1.6 Hz, 1H). Chiral separation condition: MeOH contained 0.2% Methanol Ammonia); AY-H (250*4.6 mm, 5 μm); retention time: 4.84 minutes.

Compounds 117F, 117G, and 117-1 were synthesized by employing the procedures described for Compounds 14C, 1, and 14 using Compounds 117B, 117F, 1-chloro-4-isocyanatobenzene, and 117G in lieu of Compounds 14B, 1B, 1-bromo-4-isocyanatobenzene, and 14D. Compound 117F: LC-MS (ESI) m/z: 352 [M+H]⁺. Compound 117G: LC-MS (ESI) m/z: 505 [M+H]⁺; ¹H-NMR (DMSO-d₆, 400 MHz): δ (ppm) 1.81-1.95 (m, 4H), 3.66-3.70 (m, 2H), 4.41 (s, 1H), 5.25 (s, 1H), 5.50 (d, J=4.4 Hz, 1H), 6.81 (s, 2H), 7.23-7.33 (m, 11H), 8.09 (s, 1H). Compound 117-1: LC-MS (ESI) m/z: 503 [M+H]⁺; ¹H-NMR (DMSO-d₆, 400 MHz): δ (ppm) 1.18-1.44 (m, 4H), 2.93-2.98 (m, 1H), 3.06-3.11 (m, 1H), 3.37-3.40 (m, 1H), 4.79 (d, J=5.2 Hz, 1H), 7.30-7.34 (m, 5H), 7.42-7.50 (m, 5H), 7.60 (d, J=9.2 Hz, 2H), 8.01 (s, 1H).

Compounds 117H, 117I, and 117-2 were synthesized by employing the procedures described for Compounds 14C, 1, and 14 using Compounds 117C, 117H, 1-chloro-4-isocyanatobenzene, and 117I) in lieu of Compounds 14B, 1B, 1-bromo-4-isocyanatobenzene, and 14D. Compound 117H: LC-MS (ESI) m/z: 352 [M+H]⁺. Compound 117I: LC-MS (ESI) m/z: 505 [M+H]⁺. Compound 117-2: LC-MS (ESI) m/z: 503 [M+H]⁺; ¹H-NMR (DMSO-d₆, 400 MHz): δ (ppm) 1.19-1.48 (m, 4H), 2.93-2.98 (m, 1H), 3.06-3.11 (m, 1H), 3.30-3.32 (m, 1H), 4.79 (d, J=2.8 Hz, 1H), 7.30-7.34 (m, 5H), 7.42-7.50 (m, 5H), 7.60 (d, J=8.8 Hz, 2H), 8.01 (s, 1H).

### Example 118

### Synthesis of 1,3-bis(4-chlorophenyl)-4-(3-(difluoromethoxy)phenyl)-5-ethyl-4-hydroxyimidazolidin-2-one, (5S)-1,3-bis(4-chlorophenyl)-4-(3-(difluoromethoxy)phenyl)-5-ethyl-4-hydroxyimidazolidin-2-one, and (5R)-1,3-bis(4-chlorophenyl)-4-(3-(difluoromethoxy)phenyl)-5-ethyl-4-hydroxyimidazolidin-2-one

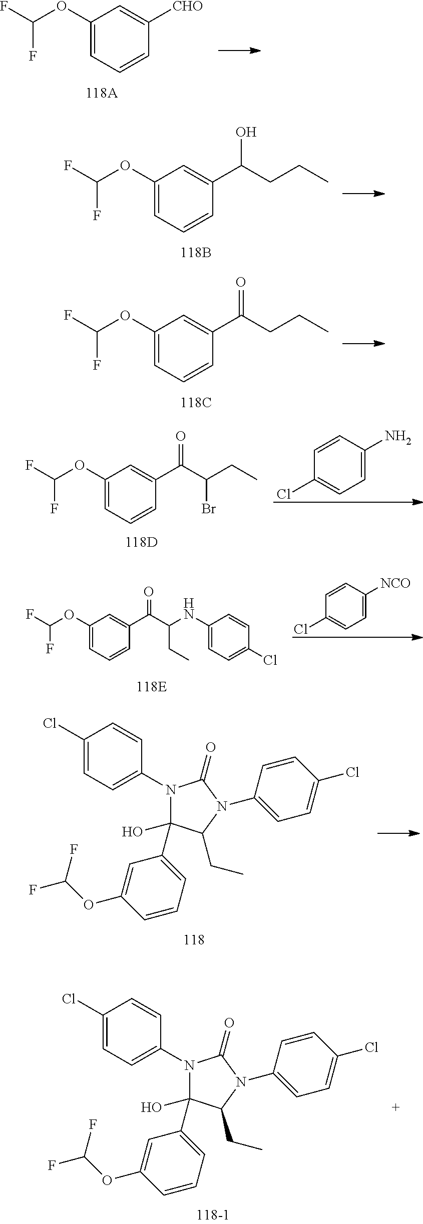

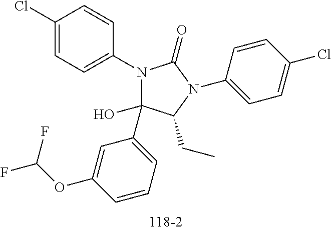 [SMILES: CC[C@H]1N(c2ccc(Cl)cc2)C(=O)N(c2ccc(Cl)cc2)C1(O)c1cccc(OC(F)F)c1]

Compounds 118B, 118C, 118D, 118E, and 118 were synthesized by employing the procedures described for Compounds 101B, 14, 13B, 1B, and 1 using Compounds 118A, propylmagnesium bromide, using THF as solvent, 118B, 118C, 118D, 4-chloroaniline, using NMP as solvent and at 50° C., 118E, and 1-chloro-4-isocyanatobenzene in lieu of Compounds 101A, n-butylmagnesium bromide, using toluene as solvent, 14D, 13A, 1A, 4-bromoaniline, using EtOH as solvent and at room temperature, 1B, and 1-bromo-4-isocyanatobenzene. Compound 118B: LC-MS (ESI) m/z: 199 [M−17]⁺; ¹H-NMR (CDCl₃, 400 MHz): δ (ppm) 0.92 (t, J=7.6 Hz, 3H), 1.28-1.44 (m, 2H), 1.59-1.76 (m, 2H), 4.63-4.65 (m, 1H), 6.51 (t, J=74 Hz, 1H), 7.00 (d, J=8.0 Hz, 1H), 7.10 (s, 1H), 7.15 (d, J=8.0 Hz, 1H), 7.31 (t, J=8.0 Hz, 1H). Compound 118C: LC-MS (ESI) m/z: 215 [M+H]⁺; ¹H-NMR (CDCl₃, 400 MHz): δ (ppm) 1.00 (t, J=7.6 Hz, 3H), 1.77 (q, J=7.6 Hz, 2H), 2.94 (t, J=7.6 Hz, 2H), 6.57 (t, J=74 Hz, 1H), 7.31-7.33 (m, 1H), 7.47 (t, J=8.0 Hz, 1H), 7.70 (s, 1H), 7.79-7.81 (m, 1H).

Compound 118D: LC-MS (ESI): m/z: 293 [M+H]⁺; ¹H-NMR (CDCl₃, 400 MHz): δ (ppm) 1.10 (t, J=7.2 Hz, 3H), 2.10-2.26 (m, 2H), 5.02 (t, J=6.8 Hz, 1H), 6.57 (t, J=74 Hz, 1H), 7.35-7.38 (m, 1H), 7.50 (t, J=8.0 Hz, 1H), 7.77 (s, 1H), 7.86 (d, J=8.0 Hz, 1H). Compound 118E: LC-MS (ESI) m/z: 340 [M+H]⁺. Compound 118: LC-MS (ESI) m/z: 493 [M+H]⁺; ¹H-NMR (CDCl₃, 400 MHz): δ (ppm) 0.73 (t, J=7.6 Hz, 3H), 1.80-1.84 (m, 2H), 3.52 (s, 1H), 4.17 (d, J=6.0 Hz, 1H), 6.47 (t, J=74 Hz, 1H), 7.03-7.26 (m, 4H), 7.28-7.38 (m, 8H).

Compound 118 was separated with chiral HPLC to give Compound 118-1 and Compound 118-2. Compound 118-1 LC-MS (ESI) m/z: 493 [M+H]⁺; ¹H-NMR (Acetone-d₆, 400 MHz): δ (ppm) 0.40, 0.74 (t, J=6.0 Hz, 3H), 1.24-2.06 (m, 2H), 4.41-4.50 (m, 1H), 6.84-7.71 (m, 14H). Chiral separation condition: MeOH contained 0.2% methanol ammonia; OD-H (4.6*250 mm, 5 μm); retention time: 1.3 minutes (74%), 2.59 minutes (26%). Compound 118-2 LC-MS (ESI) m/z: 493 [M+H]⁺; ¹H-NMR (Acetone-d₆, 400 MHz): δ (ppm) 0.40, 0.73 (t, J=6.0 Hz, 3H), 1.31-1.95 (m, 2H), 4.40-4.50 (m, 1H), 6.54-7.25 (m, 5H), 7.38-7.70 (m, 9H). Chiral separation condition: MeOH contained 0.2% methanol ammonia; OD-H (4.6*250 mm, 5 μm); retention time: 1.94 minutes (22%), 3.56 minutes (78%).

### Example 119

### Synthesis of 1,3-bis(4-bromophenyl)-4-(3-chlorophenyl)-4-hydroxy-5-propylimidazolidin-2-one, (5S)-1,3-bis(4-bromophenyl)-4-(3-chlorophenyl)-4-hydroxy-5-propylimidazolidin-2-one, and (5R)-1,3-bis(4-bromophenyl)-4-(3-chlorophenyl)-4-hydroxy-5-propylimidazolidin-2-one

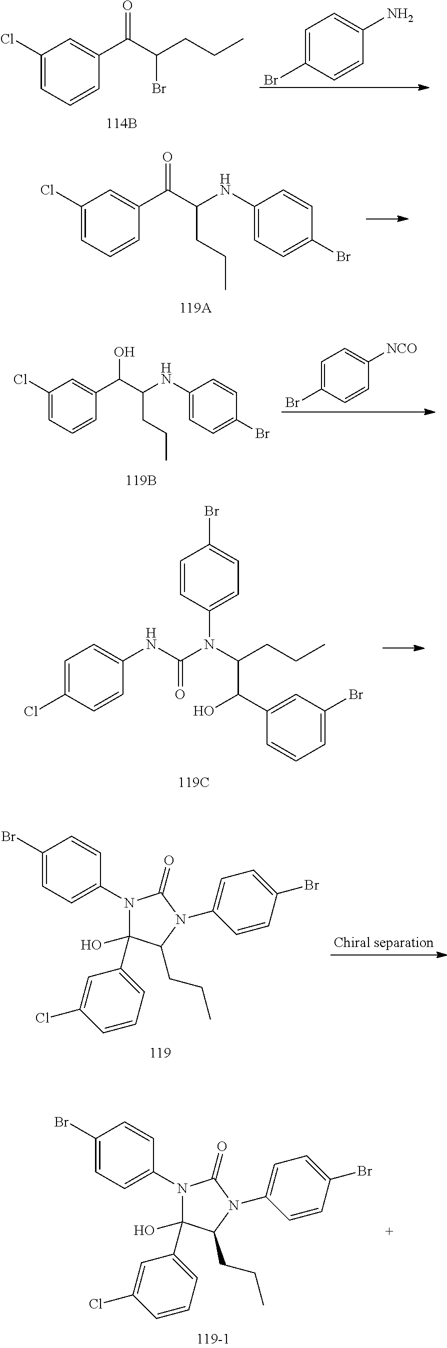

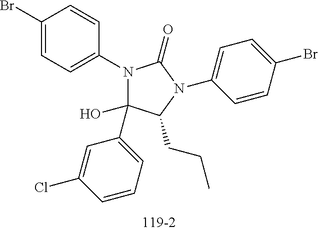 [SMILES: CCC[C@H]1N(c2ccc(Br)cc2)C(=O)N(c2ccc(Br)cc2)C1(O)c1cccc(Cl)c1]

Compounds 119A, 119B, 119C, and 119 were synthesized by employing the procedures described for Compounds 1B, 14C, 1, and 14 using 114B, 119A, 119B, and 119C in lieu of 1A, 14B, 1B, and 14D. Compound 119A: LC-MS (ESI) m/z: 366 [M+H]⁺. Compound 119B: LC-MS (ESI) m/z: 368 [M+H]⁺. Compound 119C: LC-MS (ESI) m/z: 565 [M+H]⁺. Compound 119: LC-MS (ESI) m/z: 563 [M+H]⁺; ¹H-NMR (CDCl₃, 400 MHz): δ (ppm) 0.52-0.75 (m, 3H), 0.83-1.04 (m, 2H), 1.65-2.01 (m, 2H), 3.42, 3.94 (s, 1H), 4.14-4.23 (m, 1H), 7.19-7.40 (m, 9H), 7.47-7.59 (m, 3H).

Compound 119 was separated with chiral HPLC to afford Compound 119-1 and Compound 119-2. Compound 119-1: LC-MS (ESI) m/z: 563 [M+H]⁺; ¹H-NMR (CDCl₃, 400 MHz): δ (ppm) 0.51-0.75 (m, 3H), 0.93-1.25 (m, 2H), 1.59-1.86 (m, 2H), 3.47-4.22 (m, 1H), 3.96 (s, 1H), 7.11 (d, J=8.4 Hz, 2H), 7.20-7.26 (m, 6H), 7.34-7.36 (m, 1H), 7.50 (d, J=8.8 Hz, 2H), 7.56 (s, 1H). Chiral separation condition: MeOH contained 0.2% methanol ammonia; OD-H (4.6*150 mm, 5 μm); retention time: 1.65 minutes (83.2%), 3.89 minutes 15.9%). Compound 119-2: LC-MS (ESI) m/z: 563 [M+H]⁺; ¹H-NMR (CDCl₃, 400 MHz): δ (ppm) 0.49-0.75 (m, 3H), 0.95-1.23 (m, 2H), 1.65-1.88 (m, 2H), 3.47, 4.14 (s, 1H), 4.18-4.22 (m, 1H), 7.11 (d, J=8.4 Hz, 2H), 7.20-7.26 (m, 6H), 7.34-7.36 (m, 1H), 7.49 (d, J=8.8 Hz, 2H), 7.55 (s, 1H). Chiral separation condition: MeOH contained 0.2% methanol ammonia; OD-H (4.6*150 mm, 5 μm); retention time: 2.81 minutes (17.8%), 5.98 minutes (82.1%).

### Example 120

### Synthesis of 1,3-bis(4-bromophenyl)-4-hydroxy-5,5-dimethyl-4-phenylimidazolidin-2-one

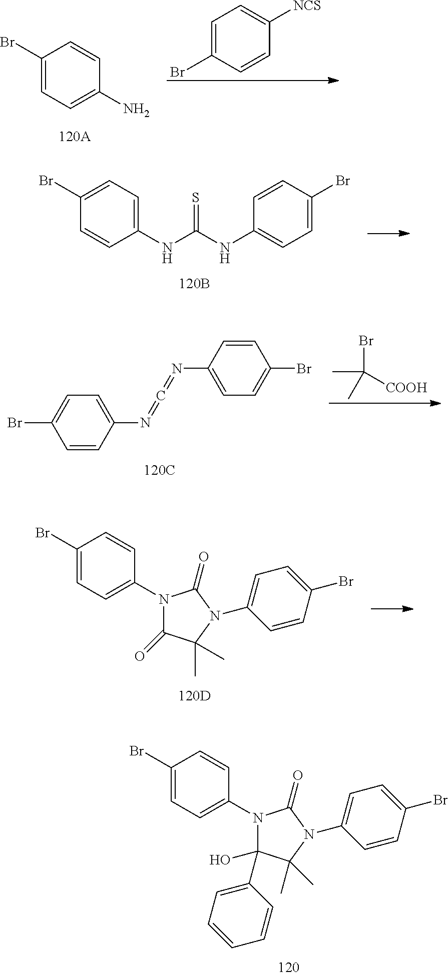

A mixture of Compound 120A (1.72 g, 10 mmol) and 1-bromo-4-isothiocynatobenzene (2.14 g, 10 mmol) in ethyl acetate (30 mL) was heated to reflux for 3 hours. The reaction mixture was concentrated under reduced pressure to furnish Compound 120B: LC-MS (ESI) m/z: 385 [M+H]⁺.

To an ice-cooled solution of Compound 120B (386 mg, 1 mmol) in ethyl acetate (5 mL) was added triethylamine (202 mg, 2 mmol), followed by addition of iodine (279 mg, 1.1 mmol) in several small portions over 30 minutes. During the addition, a light yellow solid (sulfur) was formed and filtered. The filtrate was evaporated and the residue was extracted with petroleum (15 mL×2). The combined organic layers was concentrated under reduced pressure to give Compound 120C: LC-MS (ESI) m/z: non-ionizable compound under routine conditions used; ¹H-NMR (CDCl₃, 400 MHz): δ (ppm) 7.03 (m, 4H), 7.44 (m, 4H).

To a stirred solution of 2-bromo-2-methylpropanoic acid (91 mg, 0.55 mmol) in 1,4-dioxane (4 mL) was added Compound 120C (250 mg, 0.71 mmol), followed by the addition of TMP (67 mg, 0.55 mmol) at room temperature. After stirred at room temperature overnight, to the mixture was added an aqueous solution of NaOH (2 N, 10 mL). The mixture was stirred at 0° C. until the complete conversion, acidified with 1 N HCl solution, and extracted with DCM (30 mL×2). The combined organic layers was dried over anhydrous Na₂SO₄, filtered, and concentrated under reduced pressure. The residue was purified with flash column chromatography on silica gel (ethyl acetate in petroleum ether, 20% v/v) to yield Compound 120D: LC-MS (ESI) m/z: 437 [M+H]⁺; ¹H-NMR (CDCl₃, 400 MHz): δ (ppm) 1.53 (s, 6H), 7.20 (dd, J=2.0, 6.8 Hz, 2H), 7.39 (dd, J=2.0, 6.8 Hz, 2H), 7.59-7.61 (m, 4H).

To a stirred solution of Compound 120D (70 mg, 0.159 mmol) in dry THF (3 mL) was added a solution of phenyllithium in di-n-butyl ether (1 M, 0.79 mL, 0.799 mmol) at −60° C. under nitrogen. The mixture was stirred at −60° C. for 30 minutes, quenched with saturated NH₄Cl solution (20 mL), and extracted with ethyl acetate (30 mL×2). The combined organic layers was dried over anhydrous Na₂SO₄, filtered, and concentrated under reduced pressure. The residue was purified with preparative HPLC to furnish Compound 120: LC-MS (ESI) m/z: 515 [M+H]⁺; ¹H-NMR (CDCl₃, 400 MHz): δ (ppm) 0.80 (s, 3H), 1.35 (s, 3H), 3.59 (s, 1H), 7.14 (d, J=8.8 Hz, 2H), 7.29-7.35 (m, 9H), 7.53 (d, J=8.8 Hz, 2H).

### Example 121

### Synthesis of 1,3-bis(4-chlorophenyl)-4-hydroxy-5-methyl-4,5-diphenylimidazolidin-2-one, (5S)-1,3-bis(4-chlorophenyl)-4-hydroxy-5-methyl-4,5-diphenylimidazolidin-2-one, and (5R)-1,3-bis(4-chlorophenyl)-4-hydroxy-5-methyl-4,5-diphenylimidazolidin-2-one

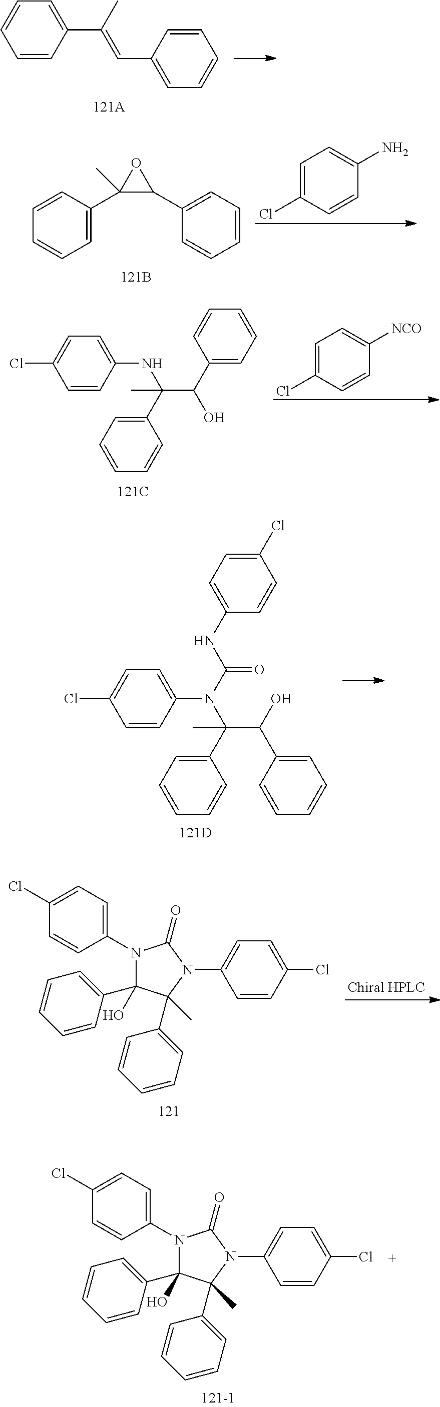

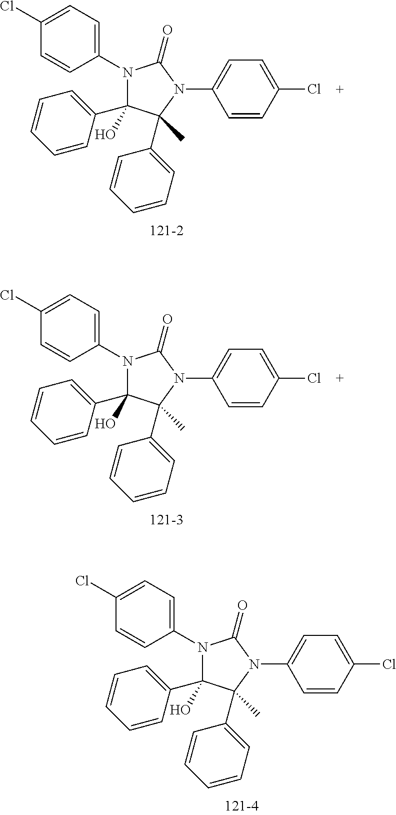 [SMILES: C[C@]1(c2ccccc2)N(c2ccc(Cl)cc2)C(=O)N(c2ccc(Cl)cc2)[C@]1(O)c1ccccc1 ; C[C@@]1(c2ccccc2)N(c2ccc(Cl)cc2)C(=O)N(c2ccc(Cl)cc2)[C@@]1(O)c1ccccc1 ; C[C@@]1(c2ccccc2)N(c2ccc(Cl)cc2)C(=O)N(c2ccc(Cl)cc2)[C@]1(O)c1ccccc1]

Compound 121B was synthesized by employing the procedure described for Compound 84B using Compound 121A in lieu of Compound 22A: LC-MS (ESI) m/z: 211 [M+H]⁺; ¹H-NMR (CDCl₃, 400 MHz): δ (ppm) 1.47 (s, 3H), 3.97 (s, 1H), 7.23-7.46 (m, 10H).

A mixture of 4-chloroaniline (2.4 g, 19 mmol) and Compound 121B (2.0 g, 9.5 mmol) was heated to 145° C. for 2 hours. After cooled down to room temperature, the reaction mixture was purified directly with flash column chromatography on silica gel (ethyl acetate in petroleum ether, from 0% to 20% v/v) to afford Compound 121C: LC-MS (ESI) m/z: 338 [M+H]⁺.

Compounds 121D and 121 were synthesized by employing the procedures described for Compounds 1 and 14 using Compounds 121C, 1-chloro-4-isocyanatobenzene, 121D, and using 1,2-dichloroethane as solvent at 80° C. in lieu of Compounds 1B, 1-bromo-4-isocyanatobenzene, 14D, and using dichloromethane as solvent at room temperature. Compound 121D: LC-MS (ESI) m/z: 491 [M+H]⁺. Compound 121: LC-MS (ESI) m/z: 489 [M+H]⁺; ¹H-NMR (CDCl₃, 400 MHz): δ (ppm) 1.20, 1.81 (s, 3H), 2.20, 3.57 (s, 1H), 6.70-7.26 (m, 10H), 7.30-7.54 (m, 8H).

Compound 121 was separated with chiral-HPLC to furnish Compound 121-1, Compound 121-2, Compound 121-3, and Compound 121-4. Compound 121-1: LC-MS (ESI) m/z: 489 [M+H]⁺; ¹H-NMR (CDCl₃, 400 MHz): δ (ppm) 1.82 (s, 3H), 3.62 (s, 1H), 6.70-6.90 (m, 5H), 7.00-7.15 (m, 8H), 7.17-7.25 (m, 3H), 7.35-7.37 (m, 2H); Chiral separation condition: MeOH contained 0.2% methanol ammonia; IC (4.6*250 mm, 5 μm); retention time: 1.18 minutes. Compound 121-2: LC-MS (ESI) m/z: 489 [M+H]⁺; ¹H-NMR (CDCl₃, 400 MHz): δ (ppm) 1.20 (s, 3H), 2.20 (s, 1H), 7.03-7.05 (m, 2H), 7.12-7.15 (m, 2H), 7.19-7.26 (m, 4H), 7.30-7.55 (m, 10H); Chiral separation condition: MeOH contained 0.2% methanol ammonia; IC (4.6*250 mm, 5 μm); retention time: 1.89 minutes. Compound 121-3: LC-MS (ESI) m/z: 489 [M+H]⁺; ¹H-NMR (CDCl₃, 400 MHz): δ (ppm) 1.20 (s, 3H), 2.20 (s, 1H), 7.03-7.05 (m, 2H), 7.12-7.15 (m, 2H), 7.19-7.26 (m, 4H), 7.30-7.55 (m, 10H); Chiral separation condition: MeOH contained 0.2% methanol ammonia; IC (4.6*250 mm, 5 μm); retention time: 2.51 minutes.

Compound 121-4: LC-MS (ESI) m/z: 489 [M+H]⁺; ¹H-NMR (CDCl₃, 400 MHz): δ (ppm) 1.82 (s, 3H), 3.53 (s, 1H), 6.70-6.90 (m, 5H), 7.00-7.15 (m, 8H), 7.17-7.25 (m, 3H), 7.35-7.37 (m, 2H); Chiral separation condition: MeOH contained 0.2% methanol ammonia; IC (4.6*250 mm, 5 μm); retention time: 3.45 minutes.

### Example 122

### Synthesis of 1,3-bis(4-chlorophenyl)-4-hydroxy-4,5-dipropylimidazolidin-2-one

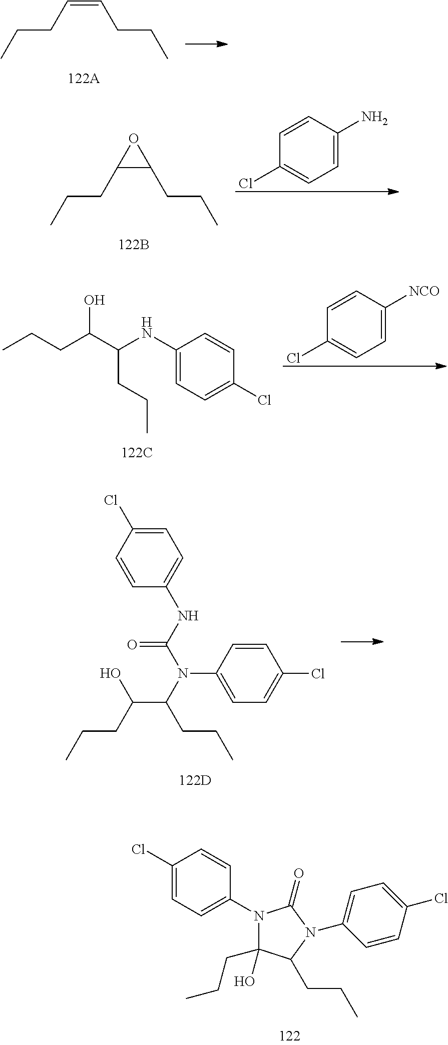

Compound 122B was synthesized by employing the procedure described for Compound 22B using Compound 122A lieu of Compound 22A: LC-MS (ESI) m/z: non-ionizable compound under routine conditions used; ¹H-NMR (CDCl₃, 400 MHz), δ (ppm) 0.96 (t, J=7.6 Hz, 6H), 1.44-1.58 (m, 8H), 2.90-2.94 (m, 2H).

To a solution of Compound 122B (300 mg, 2.3 mmol) in dichloromethane (10 mL) was added 4-chloroaniline (270 mg, 2.1 mmol) and Scandium (III) triflate (113 mg, 0.23 mmol). The mixture was stirred at 45° C. for 16 hours and concentrated under reduced pressure. The residue was purified with flash column chromatography on silica gel (ethyl acetate in petroleum ether, 10% v/v) to afford Compound 122C: LC-MS (ESI) m/z: 256 [M+H]⁺; ¹H-NMR (CDCl₃, 400 MHz): δ (ppm) 0.88-0.94 (m, 6H), 1.30-1.63 (m, 8H), 1.97 (d, J=3.6 Hz, 1H), 3.22 (s, 1H), 3.61-3.67 (m, 2H), 6.49-6.52 (m, 2H), 7.20-7.24 (m, 2H).

Compounds 122D and 122 were synthesized by employing the procedures described for Compounds 1 and 14 using Compounds 122C, 1-chloro-4-isocyanatobenzene, using 1,2-dichloroethane as solvent at 95° C., and 122D in lieu of Compounds 1B, 1-bromo-4-isocyanatobenzene, using EtOH as solvent at room temperature, and 14D. Compound 122D: LC-MS (ESI) m/z: 409 [M+H]⁺; ¹H-NMR (CDCl₃, 400 MHz): δ (ppm) 0.91-0.96 (m, 6H), 1.28-1.54 (m, 8H), 1.69-1.74 (m, 1H), 2.01 (s, 1H), 3.48 (brs, 1H), 6.01 (s, 1H), 7.18-7.24 (m, 4H), 7.33-7.38 (m, 2H), 7.46-7.48 (m, 2H). Compound 122: LC-MS (ESI) m/z: 407 [M+H]⁺; ¹H-NMR (CD₃OD, 400 MHz): δ (ppm) 0.83-0.92 (m, 6H), 1.31-1.87 (m, 8H), 4.27-4.35 (m, 1H), 7.37-7.59 (m, 8H).

### Example 123

### Synthesis of 3-(5-ethyl-1,3-bis(4-fluorophenyl)-4-hydroxy-2-oxoimidazolidin-4-yl)benzonitrile, 3-((5S)-5-ethyl-1,3-bis(4-fluorophenyl)-4-hydroxy-2-oxoimidazolidin-4-yl)benzonitrile, and 3-((5R)-5-ethyl-1,3-bis(4-fluorophenyl)-4-hydroxy-2-oxoimidazolidin-4-yl)benzonitrile

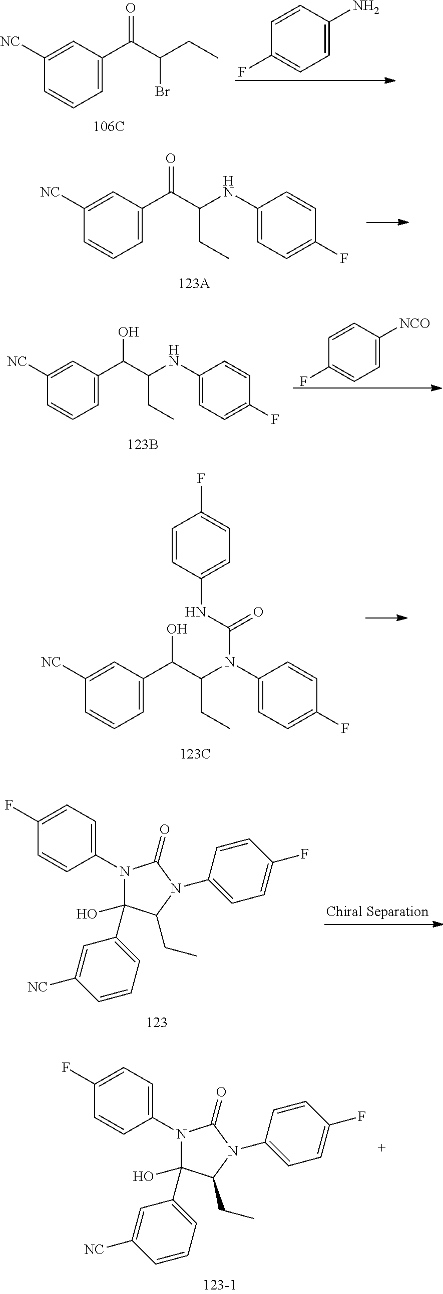

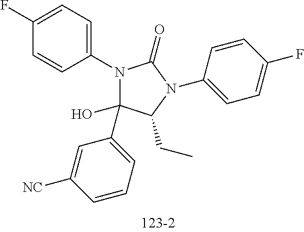 [SMILES: CC[C@H]1N(c2ccc(F)cc2)C(=O)N(c2ccc(F)cc2)C1(O)c1cccc(C#N)c1]

Compounds 123A, 123B, 123C, and 123 were synthesized by employing the procedures described for Compounds 1B, 14C, 1, and 14 using 106C, 4-fluoroaniline, using NMP as solvent, 123A, 123B, 1-floro-4-isocyanatobenzene, and 123C in lieu of 1A, 4-bromoaniline, using EtOH as solvent, 14B, 1B, 1-bromo-4-isocyanatobenzene, and 14D. Compound 123A: LC-MS (ESI) m/z: 283 [M+H]⁺; ¹H-NMR (CDCl₃, 400 MHz): δ (ppm) 0.94 (t, J=7.2 Hz, 3H), 1.71-1.73 (m, 1H), 2.01-2.05 (m, 1H), 4.89-4.92 (m, 1H), 6.60-6.64 (m, 2H), 6.88-6.91 (m, 2H), 7.63-7.67 (m, 1H), 7.87-7.89 (m, 1H), 8.19-8.21 (m, 1H), 8.26 (s, 1H). Compound 123B: LC-MS (ESI) m/z: 285 [M+H]⁺; ¹H-NMR (CDCl₃, 400 MHz): δ (ppm) 0.89 (t, J=7.2 Hz, 3H), 1.27-1.39 (m, 1H), 1.60-1.65 (m, 1H), 2.58-2.60 (m, 1H), 3.10-3.45 (m, 2H), 4.95 (s, 1H), 6.57-6.66 (m, 2H), 6.85-6.93 (m, 2H), 7.45-7.49 (m, 1H), 7.56-7.63 (m, 2H), 7.69-7.70 (m, 1H). Compound 123C: LC-MS (ESI) m/z: 422 [M+H]⁺.

The crude product was purified with chiral HPLC to give Compound 123-1 and Compound 123-2. Compound 123-1: LC-MS (ESI) m/z: 420 [M+H]⁺; ¹H-NMR (CD₃OD, 400 MHz): δ (ppm) 0.52 (t, J=7.2 Hz, 3H), 1.55-1.78 (m, 2H), 4.25-4.29 (m, 1H), 6.81-6.86 (m, 2H), 7.05-7.10 (m, 2H), 7.23-7.35 (m, 4H), 7.36-7.42 (m, 1H), 7.50-7.52 (m, 1H), 7.91-7.99 (m, 2H). Chiral separation condition: MeOH contained 0.2% methanol ammonia; OJ-H (4.6*250 mm, 5 μm); retention time: 1.85 minutes (90%), 2.9 minutes (10%). Compound 123-2: LC-MS (ESI) m/z: 420 [M+H]⁺; ¹H-NMR (CD₃OD, 400 MHz): δ (ppm) 0.63 (t, J=7.6 Hz, 3H), 1.68-1.87 (m, 2H), 4.36-4.40 (m, 1H), 6.92-6.97 (m, 2H), 7.16-7.21 (m, 2H), 7.34-7.37 (m, 2H), 7.44-7.48 (m, 2H), 7.49-7.63 (m, 2H), 8.02-8.10 (m, 2H). Chiral separation condition: Chiral separation condition: MeOH contained 0.2% methanol ammonia; OJ-H (4.6*250 mm, 5 m); retention time: 2.13 minutes (87%), 2.31 minutes (13%).

### Example 124

### Synthesis of 4-(3-acetylphenyl)-1,3-bis(4-chlorophenyl)-4-hydroxy-5-methylimidazolidin-2-one

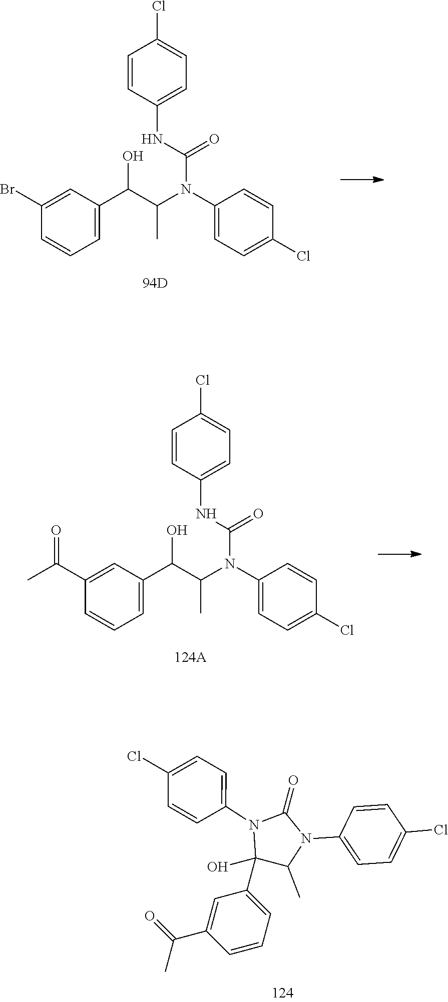 [SMILES: CC(C(O)c1cccc(Br)c1)N(C(=O)Nc1ccc(Cl)cc1)c1ccc(Cl)cc1 ; CC(=O)c1cccc(C(O)C(C)N(C(=O)Nc2ccc(Cl)cc2)c2ccc(Cl)cc2)c1 ; CC(=O)c1cccc(C2(O)C(C)N(c3ccc(Cl)cc3)C(=O)N2c2ccc(Cl)cc2)c1]

Compounds 124A and 124 were synthesized by employing the procedures described for Compounds 47B and 14 using Compounds 94D and 124A in lieu of Compounds 47A and 14D. Compound 124A: LC-MS (ESI) m/z: 457 [M+H]⁺. Compound 124: LC-MS (ESI) m/z: 455 [M+H]⁺; ¹H-NMR (acetone-d₆, 400 MHz): δ (ppm) 1.29 (d, J=5.2 Hz, 3H), 2.55 (s, 3H), 4.50-4.55 (m, 1H), 6.55 (s, 1H), 7.17-7.21 (m, 2H), 7.40-7.55 (m, 7H), 7.90-7.96 (m, 2H), 8.32-8.36 (m, 1H).

### Example 125

### Synthesis of 1,3-bis(4-bromophenyl)-4-(3-(dimethylamino)phenyl)-4-hydroxyimidazolidin-2-one

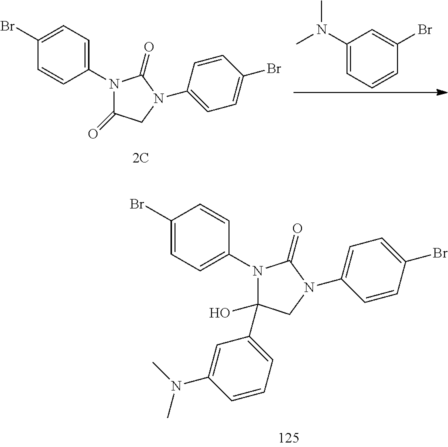

To a solution of 3-bromo-N,N-dimethylaniline (600 mg, 3.0 mmol) in THF (10 mL) was dropped a solution of n-BuLi in hexane (2.5 M, 1 mL, 2.5 mmol) at −60° C. under nitrogen atmosphere and stirred at −60° C. for 30 minutes. The mixture was dropped into a solution of Compound 2C (205 mg, 0.5 mmol) in THF (10 mL) at −60° C. The mixture was stirred at −60° C. for 30 minutes, quenched with saturated NH₄Cl solution (10 mL), and extracted with ethyl acetate (20 mL×2). The combined organic layers was dried over anhydrous sodium sulfate, filtered, and evaporated under reduced pressure. The residue was purified by preparative HPLC to give Compound 125: LC-MS (ESI) m/z: 530 [M+H]⁺; ¹H-NMR (DMSO-d₆, 400 MHz): δ (ppm) 2.83 (s, 6H), 3.98-4.09 (m, 2H), 6.65-6.59 (m, 1H), 6.80-6.89 (m, 2H), 7.08-7.11 (m, 1H), 7.35-7.41 (m, 4H), 7.51-7.55 (m, 3H), 7.62-7.65 (m, 2H).

### Example 126

### Synthesis of 1,3-bis(4-bromophenyl)-5-hydroxy-4-(3-methoxyphenyl)-4-methylimidazolidin-2-one, (4S)-1,3-bis(4-bromophenyl)-5-hydroxy-4-(3-methoxyphenyl)-4-methylimidazolidin-2-one, and (4R)-1,3-bis(4-bromophenyl)-5-hydroxy-4-(3-methoxyphenyl)-4-methylimidazolidin-2-one

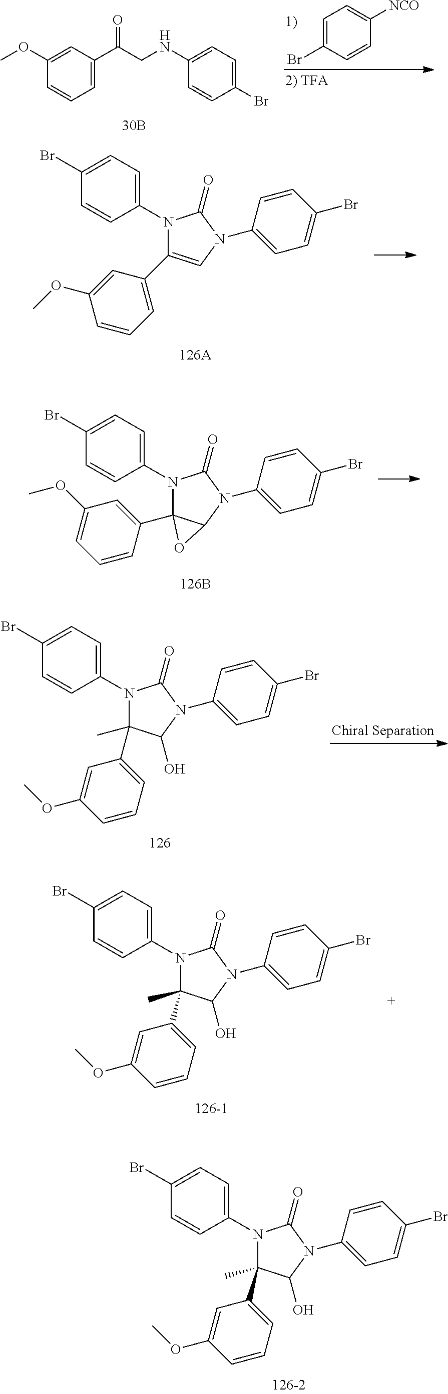

Compounds 126A, 126B, and 126 were synthesized by employing the procedures described for Compounds 86A, 86B, and 86 using Compounds 30B, 126A, and 126B in lieu of Compounds 1B, 86A, and 86B. Compound 126A. LC-MS (ESI) m/z: 499 [M+H]⁺. Compound 126B. LC-MS (ESI) m/z: 515 [M+H]⁺; ¹H-NMR (CDCl₃, 400 MHz): δ (ppm) 3.79 (s, 3H), 5.52 (s, 1H), 6.89-6.96 (m, 3H), 7.30-7.37 (m, 3H), 7.40-7.42 (m, 1H), 7.57-7.62 (m, 3H), 7.97-8.07 (m, 2H). Compound 126. LC-MS (ESI) m/z: 531 [M+H]⁺; ¹H-NMR (DMSO-d₆, 400 MHz): δ (ppm) 0.84 (s, 3H), 3.75 (s, 3H), 5.26 (s, 1H), 6.86-6.88 (m, 2H), 7.10 (s, 1H), 7.25-7.32 (m, 2H), 7.37-7.44 (m, 4H), 7.54-7.63 (m, 4H).

Compound 126 was separated with chiral HPLC to afford Compound 126-1 and Compound 126-2. Compound 126-1: LC-MS (ESI) m/z: 531 [M+H]⁺; ¹H-NMR (CD₃OD, 400 MHz): δ (ppm) 0.96 (s, 3H), 3.78 (s, 3H), 5.21 (s, 1H), 6.89-6.91 (m, 3H), 7.29-7.33 (m, 1H), 7.36-7.38 (m, 4H), 7.47-7.50 (m, 2H), 7.58-7.61 (m, 2H). Chiral separation condition: MeOH contained 0.2% Methanol Ammonia; OZ-H (250×4.6 mm, 5 μm); retention time: 2.84 minutes. Compound 126-2: LC-MS (ESI) m/z: 531 [M+H]⁺; ¹H-NMR (CD₃OD, 400 MHz): δ (ppm) 0.96 (s, 3H), 3.78 (s, 3H), 5.21 (s, 1H), 6.89-6.91 (m, 3H), 7.29-7.33 (m, 1H), 7.35-7.38 (m, 4H), 7.47-7.50 (m, 2H), 7.58-7.61 (m, 2H). Chiral separation condition: MeOH contained 0.2% Methanol Ammonia; OZ-H (250×4.6 mm, 5 μm); retention time: 3.48 minutes.

### Example 127

### Synthesis of 1,3-bis(4-bromophenyl)-4-(3-(tert-butyl)phenyl)-4-hydroxyimidazolidin-2-one

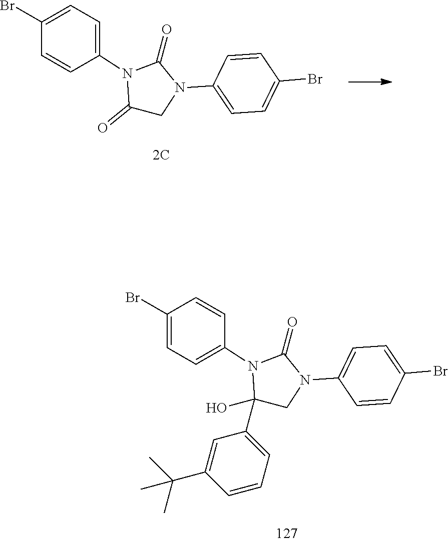 [SMILES: O=C1CN(c2ccc(Br)cc2)C(=O)N1c1ccc(Br)cc1 ; CC(C)(C)c1cccc(C2(O)CN(c3ccc(Br)cc3)C(=O)N2c2ccc(Br)cc2)c1]

To a solution of 1-bromo-3-tert-butylbenzene (266 mg, 1.25 mmol) in dry THF (5 mL) was dropped a solution of n-butyllithium in hexane (2.5 M, 0.5 mL, 1.25 mmol) at −78° C. under nitrogen and stirred for 10 minutes. The solution was slowly added into a solution of Compound 2C (100 mg, 0.25 mmol) in dry THF (5 mL) at −78° C. under nitrogen. The reaction mixture was stirred at −78° C. for 1 hour, quenched with saturated aqueous NH₄Cl solution (50 mL), and extracted with ethyl acetate (50 mL×2). The combined organic layers was washed with brine (50 mL), dried over anhydrous sodium sulfate, filtered, and concentrated under reduced pressure. The residue was purified with preparative HPLC to furnish Compound 127. LC-MS (ESI) m/z: 543 [M+H]⁺; ¹H-NMR (acetone-d₆, 400 MHz): δ (ppm) 1.24 (s, 9H), 4.12-4.27 (m, 2H), 6.67 (s, 1H), 7.24-7.38 (m, 4H), 7.42-7.54 (m, 5H), 7.66-7.76 (m, 3H).

### Example 128

### Synthesis of 4-(3-(tert-butyl)phenyl)-1,3-bis(4-chlorophenyl)-4-hydroxyimidazolidin-2-one

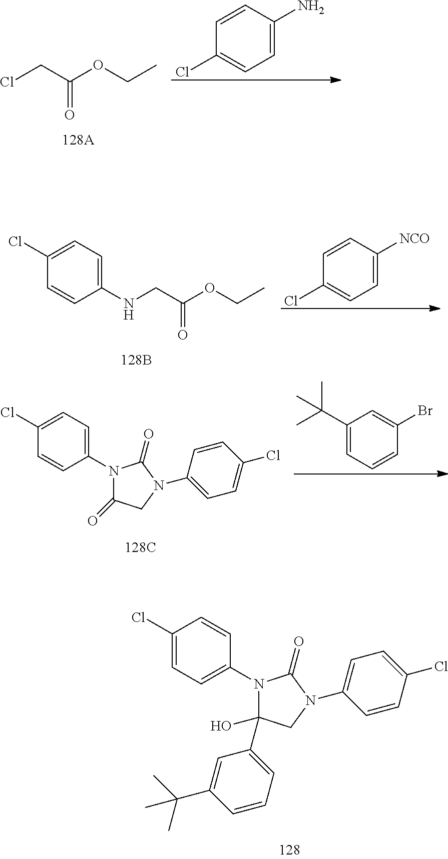

Compound 128B was synthesized by employing the procedure described for Compound 2B using Compound 128A and 4-chloroaniline in lieu of Compound 2A and 4-bromoaniline. LC-MS (ESI) m/z: 214 [M+H]⁺.

To a solution of Compound 128B (426 mg, 2.0 mmol) in pyridine (5 mL) was added 1-chloro-4-isocyanatobenzene (306 mg, 2.0 mmol). The mixture was stirred at room temperature for 5 hours and concentrated under reduced pressure. The residue was washed with ethyl acetate (5 mL) to afford Compound 128C. LC-MS (ESI) m/z: 321 [M+H]⁺; ¹H-NMR (DMSO-d₆, 400 MHz): δ (ppm) 4.61 (s, 2H), 7.45-7.50 (m, 4H), 7.59-7.61 (m, 2H), 7.70-7.73 (m, 2H).

Compound 128 was synthesized by employing the procedure described for Compound 127 using Compound 128C in lieu of Compound 2C. LC-MS (ESI) m/z: 455 [M+H]⁺; ¹H-NMR (DMSO-d₆, 400 MHz): δ (ppm) 1.18 (s, 9H), 4.02-4.15 (m, 2H), 7.22-7.26 (m, 4H), 7.35-7.43 (m, 5H), 7.56 (s, 1H), 7.64 (s, 1H), 7.71 (d, J=8.8 Hz, 2H).

### Example 129

### Synthesis of 1,3-bis(4-chlorophenyl)-4-hydroxy-5-methyl-4-(3-(trifluoromethoxy)phenyl)imidazolidin-2-one, (5S)-1,3-bis(4-chlorophenyl)-4-hydroxy-5-methyl-4-(3-(trifluoromethoxy)phenyl)imidazolidin-2-one, and (5R)-1,3-bis(4-chlorophenyl)-4-hydroxy-5-methyl-4-(3-(trifluoromethoxy)phenyl)imidazolidin-2-one

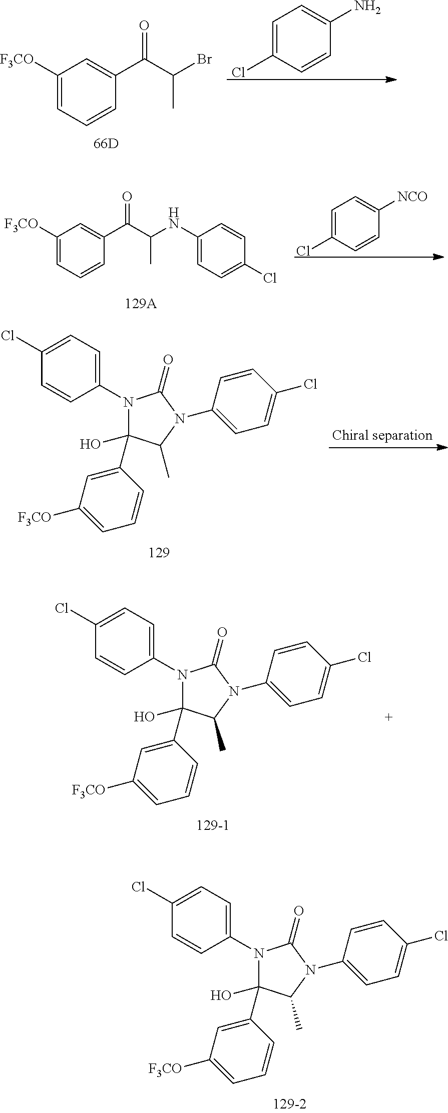

Compounds 129A and 129 were synthesized by employing the procedures described for Compounds 1B and 1 using Compounds 66D, 4-cholroaniline, stirred at 60° C., 129A, and 1-chloro-4-isocyanatobenzene in lieu of Compounds 1A, 4-bromoaniline, stirred at room temperature, 1B, and 1-bromo-4-isocyanatobenzene. Compound 129A. LC-MS (ESI) m/z: 344 [M+H]⁺; ¹H-NMR (CDCl₃, 400 MHz): δ (ppm) 1.48 (d, J=7.2 Hz, 3H), 4.67 (s, 1H), 5.01-5.08 (m, 1H), 6.56 (d, J=8.8 Hz, 2H), 7.13 (d, J=8.4 Hz, 2H), 7.47-7.48 (m, 1H), 7.57 (t, J=8.4 Hz, 1H), 7.84 (s, 1H), 7.93 (d, J=8.0 Hz, 1H). Compound 129. LC-MS (ESI) m/z: 497 [M+H]⁺; ¹H-NMR (CDCl₃, 400 MHz): δ (ppm) 0.65, 1.22 (d, J=6.4 Hz, 3H), 3.80, 5.03 (s, 1H), 4.10-4.30 (m, 1H), 7.06-7.24 (m, 6H), 7.28-7.42 (m, 6H).

Compound 129 was separated with chiral HPLC to give Compounds 129-1 and Compound 129-2. Compound 129-1: LC-MS (ESI) m/z: 497 [M+H]⁺; ¹H-NMR (DMSO-d₆, 400 MHz): δ (ppm) 0.65, 1.08 (d, J=6.4 Hz, 3H), 4.38-4.56 (m, 1H), 7.25-7.29 (m, 3H), 7.40-7.91 (m, 10H); Chiral separation condition: MeOH contained 0.2% methanol ammonia; OD-H (4.6*150 mm, 5 μm); retention time: 1.25 minutes (80%), 2.52 minutes (20%). Compound 129-2: LC-MS (ESI) m/z: 497 [M+H]⁺; ¹H-NMR (DMSO-d₆, 400 MHz): δ (ppm) 0.65, 1.09 (d, J=6.8 Hz, 3H), 4.38-4.57 (m, 1H), 7.25-7.31 (m, 3H), 7.42-7.91 (m, 10H); Chiral separation condition: MeOH contained 0.2% methanol ammonia; OD-H (4.6*150 mm, 5 μm); retention time: 2.10 minutes (19%), 3.76 minutes (81%).

### Example 130

### Synthesis of 1,3-bis(4-fluorophenyl)-4-hydroxy-5-methyl-4-(3-(trifluoromethoxy)phenyl)imidazolidin-2-one, (5S)-1,3-bis(4-fluorophenyl)-4-hydroxy-5-methyl-4-(3-(trifluoromethoxy)phenyl)imidazolidin-2-one, and (5R)-1,3-bis(4-fluorophenyl)-4-hydroxy-5-methyl-4-(3-(trifluoromethoxy)phenyl)imidazolidin-2-one

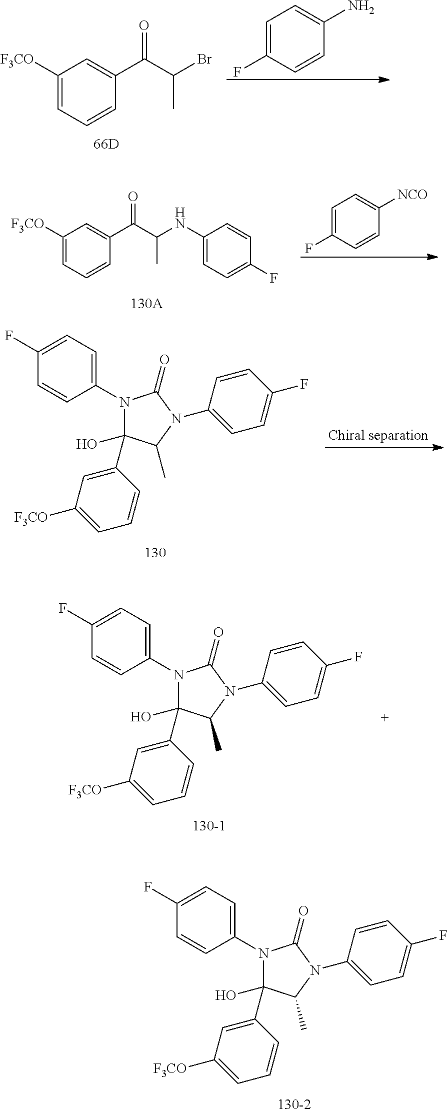

Compounds 130A and 130 were synthesized by employing the procedures described for Compounds 1B and 1 using Compounds 66D, 4-fluoroaniline, stirred at 60° C., 130A, and 1-fluoro-4-isocyanatobenzene in lieu of Compounds 1A, 4-bromoaniline, stirred at room temperature, 1B, and 1-bromo-4-isocyanatobenzene. Compound 130A. LC-MS (ESI) m/z: 328 [M+H]⁺; ¹H-NMR (CDCl₃, 400 MHz): δ (ppm) 1.48 (d, J=7.2 Hz, 3H), 4.67 (s, 1H), 4.97-5.05 (m, 1H), 6.59-6.62 (m, 2H), 6.89 (t, J=8.8 Hz, 2H), 7.47 (d, J=8.0 Hz, 1H), 7.56 (t, J=8.4 Hz, 1H), 7.84 (s, 1H), 7.93 (d, J=8.4 Hz, 1H). Compound 130. LC-MS (ESI) m/z: 465 [M+H]⁺; ¹H-NMR (CDCl₃, 400 MHz): δ (ppm) 0.73, 1.22 (d, J=6.4 Hz, 3H), 3.56, 4.16 (s, 1H), 4.17-4.32 (m, 1H), 6.85-6.90 (m, 2H), 6.92-7.15 (m, 3H), 7.25-7.28 (m, 2H), 7.33-7.44 (m, 5H).

Compound 130 was separated with chiral HPLC to give Compounds 130-1 and Compound 130-2. Compound 130-1: LC-MS (ESI) m/z: 465 [M+H]⁺; ¹H-NMR (DMSO-d₆, 400 MHz): δ (ppm) 0.63, 1.06 (d, J=6.8 Hz, 3H), 4.34-4.53 (m, 1H), 7.03-7.09 (m, 2H), 7.11-7.27 (m, 3H), 7.37-7.81 (m, 8H); Chiral separation condition: MeOH contained 0.2% methanol ammonia; OD-H (4.6*150 mm, 5 μm); retention time: 1.78 minutes (82%), 4.76 minutes (18%). Compound 130-2: LC-MS (ESI) m/z: 465 [M+H]⁺; ¹H-NMR (DMSO-d₆, 400 MHz): δ (ppm) 0.63, 1.07 (d, J=6.8 Hz, 3H), 4.36-4.52 (m, 1H), 7.03-7.11 (m, 2H), 7.22-7.29 (m, 3H), 7.37-7.81 (m, 8H); Chiral separation condition: MeOH contained 0.2% methanol ammonia; OD-H (4.6*150 mm, 5 μm); retention time: 3.45 minutes (68%), 5.95 minutes (32%).

### Example 131

### Synthesis of 5-ethyl-1,3-bis(4-fluorophenyl)-4-hydroxy-4-(3-(trifluoromethoxy)phenyl)imidazolidin-2-one

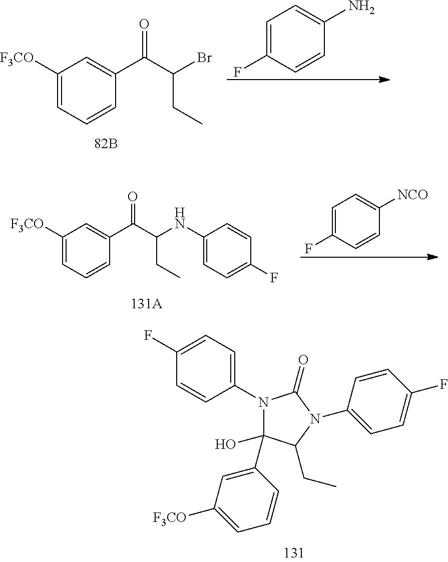

Compounds 131A and 131 were synthesized by employing the procedures described for Compounds 1B and 1 using Compounds 82B, 4-fluoroaniline, stirred at 60° C., 131A, and 1-fluoro-4-isocyanatobenzene in lieu of Compounds 1A, 4-bromoaniline, stirred at room temperature, 1B, and 1-bromo-4-isocyanatobenzene. Compound 131A. LC-MS (ESI) m/z: 342 [M+H]⁺. Compound 131. LC-MS (ESI) m/z: 479 [M+H]⁺; ¹H-NMR (CD₃OD, 400 MHz): δ (ppm) 0.44, 0.66 (t, J=7.2 Hz, 3H), 1.20-1.41 (m, 1H), 1.82-1.86 (m, 1H), 4.26-4.38 (m, 1H), 6.91-6.99 (m, 2H), 7.17-7.23 (m, 3H), 7.33-7.35 (m, 1H), 7.36-7.56 (m, 5H), 7.69-7.71 (m, 1H).

### Example 132

### Synthesis of 3-(1,3-bis(4-chlorophenyl)-5-(cyclopropylmethyl)-4-hydroxy-2-oxoimidazolidin-4-yl)benzonitrile, 3-((5S)-1,3-bis(4-chlorophenyl)-5-(cyclopropylmethyl)-4-hydroxy-2-oxoimidazolidin-4-yl)benzonitrile, 3-((5R)-1,3-bis(4-chlorophenyl)-5-(cyclopropylmethyl)-4-hydroxy-2-oxoimidazolidin-4-yl)benzonitrile

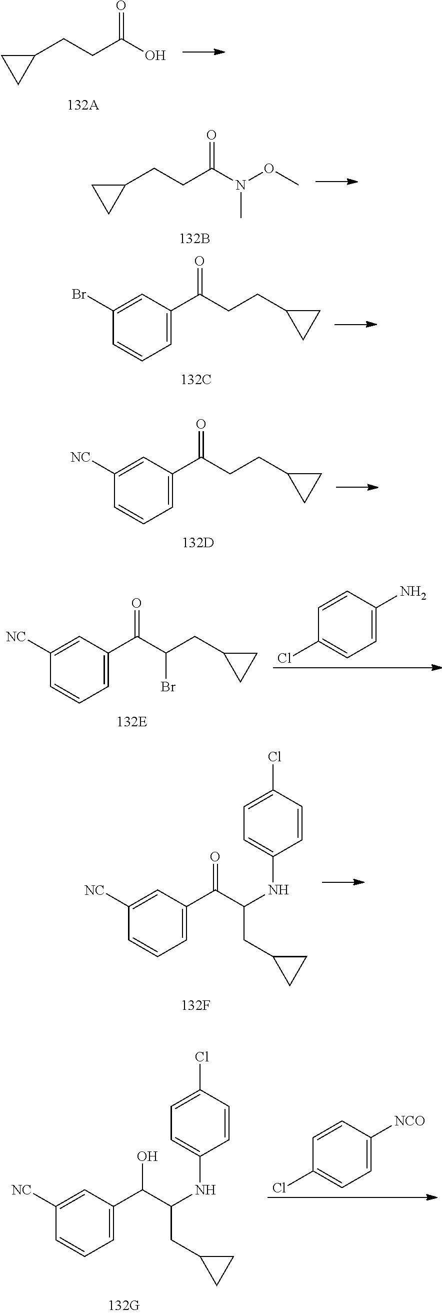

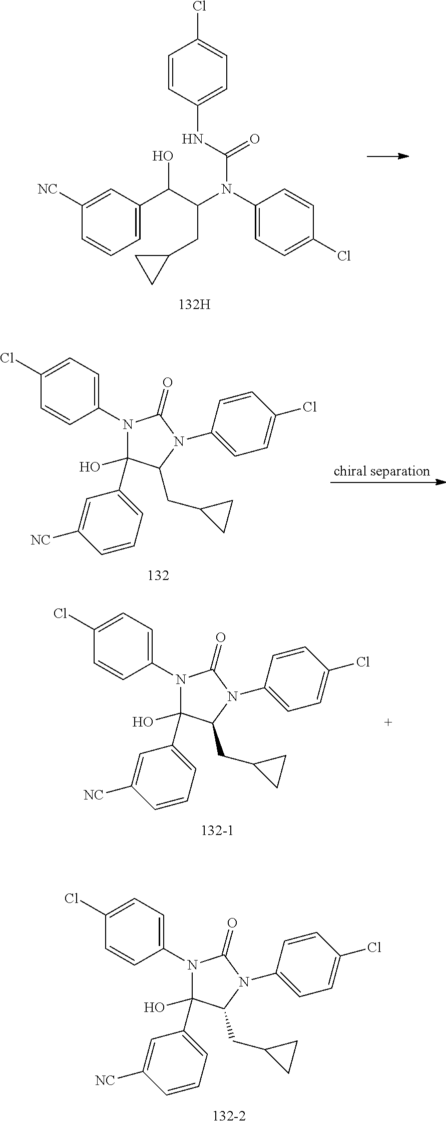 [SMILES: N#Cc1cccc(C(O)C(CC2CC2)N(C(=O)Nc2ccc(Cl)cc2)c2ccc(Cl)cc2)c1 ; N#Cc1cccc(C2(O)C(CC3CC3)N(c3ccc(Cl)cc3)C(=O)N2c2ccc(Cl)cc2)c1 ; N#Cc1cccc(C2(O)[C@H](CC3CC3)N(c3ccc(Cl)cc3)C(=O)N2c2ccc(Cl)cc2)c1 ; N#Cc1cccc(C2(O)[C@@H](CC3CC3)N(c3ccc(Cl)cc3)C(=O)N2c2ccc(Cl)cc2)c1]

Compounds 132B, 132C, 132D, 132E, 132F, 132G, 132H, and 132 were synthesized by employing the procedures described for Compounds 98B, 59B, 95B, 13B, 1B, 14C, 1, and 14 using Compounds 132A, 132B, 1,3-dibromobenzene, 132C, 132D, 132E, 4-chloroaniline, 132F, 132G, 1-chloro-4-isocyanatobenzene, and 132H) in lieu of Compounds 98A, N-methoxy-N-methylacetamide, 59A, 95A, 13A, 1A, 4-bromoaniline, 14B, 1B, 1-bromo-4-isocyanatobenzene, and 14D. Compound 132B. LC-MS (ESI) m/z: 158 [M+H]⁺; ¹H-NMR (CDCl₃, 400 MHz): δ (ppm) 0.06-0.10 (m, 2H), 0.42-0.45 (m, 2H), 0.71-0.78 (m, 1H), 1.52-1.58 (m, 2H), 2.53-2.56 (m, 2H), 3.20 (s, 3H), 3.72 (s, 3H). Compound 132C. LC-MS (ESI) m/z: non-ionizable compound under routine conditions used; ¹H-NMR (CDCl₃, 400 MHz): δ (ppm) 0.07-0.11 (m, 2H), 0.45-0.49 (m, 2H), 0.74-0.81 (m, 1H), 1.65 (q, J=7.2 Hz, 2H), 3.03 (t, J=7.2 Hz, 2H), 7.36 (t, J=8.0 Hz, 1H), 7.69 (d, J=8.0 Hz, 1H), 7.90 (d, J=8.0 Hz, 1H), 8.11 (s, 1H). Compound 132D. LC-MS (ESI) m/z: non-ionizable compound under routine conditions used; ¹H-NMR (CDCl₃, 400 MHz): δ (ppm) 0.07-0.11 (m, 2H), 0.45-0.49 (m, 2H), 0.74-0.81 (m, 1H), 1.66 (q, J=7.2 Hz, 2H), 3.10 (t, J=7.2 Hz, 2H), 7.62 (t, J=8.0 Hz, 1H), 7.85 (d, J=8.0 Hz, 1H), 8.21 (d, J=8.0 Hz, 1H), 8.26 (s, 1H). Compound 132E. LC-MS (ESI) m/z: non-ionizable compound under routine conditions used; ¹H-NMR (CDCl₃, 400 MHz): δ (ppm) 0.14-0.27 (m, 2H), 0.51-0.56 (m, 2H), 0.84-0.89 (m, 1H), 2.02-2.09 (m, 1H), 2.15-2.21 (m, 1H), 5.14 (t, J=6.8 Hz, 1H), 7.66 (t, J=8.0 Hz, 1H), 7.89 (d, J=8.0 Hz, 1H), 8.27 (d, J=8.0 Hz, 1H), 8.33 (s, 1H). Compound 132F. LC-MS (ESI) m/z: 325 [M+H]⁺. Compound 132G. LC-MS (ESI) m/z: 327 [M+H]⁺. Compound 132H. LC-MS (ESI) m/z: 480 [M+H]⁺; ¹H-NMR (CDCl₃, 400 MHz): δ (ppm) −0.13-−0.09 (m, 1H), 0.15-0.19 (m, 1H), 0.40-0.46 (m, 1H), 0.50-0.55 (m, 1H), 0.67-0.72 (m, 1H), 1.21-1.25 (m, 1H), 2.37-2.46 (m, 1H), 3.57-3.60 (m, 1H), 5.35 (s, 1H), 5.74 (s, 1H), 6.02 (s, 1H), 7.24-7.26 (m, 4H), 7.33-7.35 (m, 2H), 7.42-7.44 (m, 1H), 7.51-7.58 (m, 4H), 7.63 (s, 1H). Compound 132. LC-MS (ESI) m/z: 478 [M+H]⁺; ¹H-NMR (CDCl₃, 400 MHz): δ (ppm) −0.19-−0.14 (m, 1H), −0.09-−0.03 (m, 1H), 0.04-0.11 (m, 1H), 0.29-0.37 (m, 2H), 1.30-1.37 (m, 1H), 1.88-1.96 (m, 1H), 4.28-4.38 (m, 2H), 7.09 (d, J=8.8 Hz, 2H), 7.20-7.22 (m, 4H), 7.33 (d, J=8.8 Hz, 2H), 7.43 (t, J=8.0 Hz, 1H), 7.57 (d, J=8.0 Hz, 1H), 7.76 (d, J=8.0 Hz, 1H), 7.94 (s, 1H).

Compound 132 was separated with chiral HPLC to yield Compounds 132-1 and Compound 132-2. Compound 132-1: LC-MS (ESI) m/z: 478 [M+H]⁺; ¹H-NMR (CDCl₃, 400 MHz): δ (ppm) −0.17-−0.11 (m, 1H), −0.06-−0.02 (m, 1H), 0.08-0.14 (m, 1H), 0.31-0.41 (m, 2H), 1.36-1.43 (m, 1H), 1.94-2.00 (m, 1H), 4.14 (s, 1H), 4.31-4.34 (m, 1H), 7.10 (d, J=8.8 Hz, 2H), 7.20-7.26 (m, 4H), 7.36 (d, J=8.8 Hz, 2H), 7.44 (t, J=8.0 Hz, 1H), 7.57 (d, J=8.0 Hz, 1H), 7.76 (d, J=8.0 Hz, 1H), 7.95 (s, 1H). Chiral separation condition: MeOH contained 0.2% Methanol Ammonia; OD-H (4.6*100 mm 5 μm); retention time: 1.30 minutes. Compound 132-2: LC-MS (ESI) m/z: 478 [M+H]⁺; ¹H-NMR (CDCl₃, 400 MHz): δ (ppm) −0.16-−0.11 (m, 1H), −0.06-−0.02 (m, 1H), 0.09-0.13 (m, 1H), 0.31-0.40 (m, 2H), 1.36-1.42 (m, 1H), 1.95-2.02 (m, 1H), 4.24 (s, 1H), 4.31-4.34 (m, 1H), 7.09 (d, J=8.8 Hz, 2H), 7.19 (d, J=8.8 Hz, 2H), 7.25 (d, J=8.8 Hz, 2H), 7.35 (d, J=8.8 Hz, 2H), 7.43 (d, J=8.0 Hz, 1H), 7.57 (d, J=8.0 Hz, 1H), 7.77 (d, J=8.0 Hz, 1H), 7.94 (s, 1H). Chiral separation condition: MeOH contained 0.2% Methanol Ammonia; OD-H (4.6*100 mm 5 μm); retention time: 4.12 minutes.

### Example 133

### Synthesis of 1,3-bis(4-chlorophenyl)-4-hydroxy-5,5-dimethyl-4-(3-(trifluoromethoxy)phenyl)imidazolidin-2-one

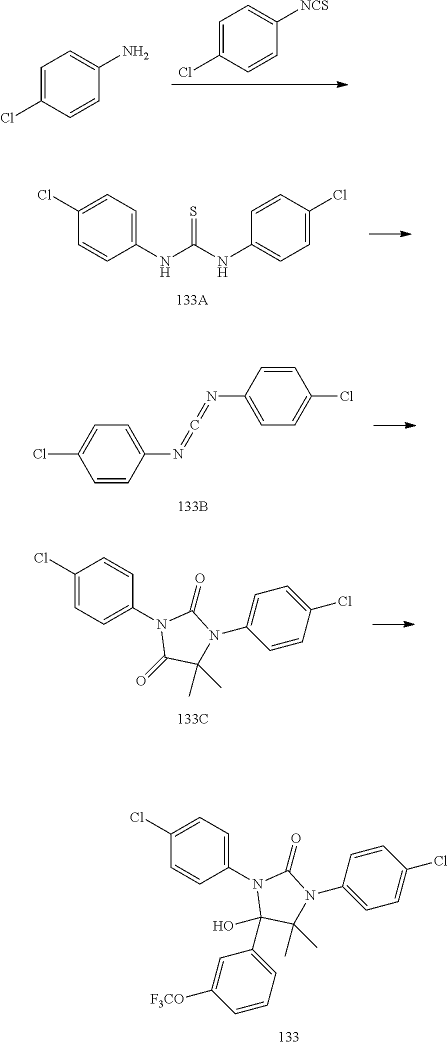

Compounds 133A, 133B, 133C, and 133 were synthesized by employing the procedures described for Compounds 120B, 120C, 120D, and 127 using 4-chloroaniline, 1-chloro-4-isothiocynatobenzene, Compounds 133A, 133B, 133C, and 1-bromo-3-(trifluoromethoxy)benzene in lieu of Compounds 120A, 1-bromo-4-isocyanatobenzene, 120B, 120C, 2C, and 1-bromo-3-tert-butylbenzene. Compound 133A. LC-MS (ESI) m/z: 297 [M+H]⁺. Compound 133B. LC-MS (ESI) m/z: non-ionizable compound under routine conditions used; ¹H-NMR (400 MHz, CDCl₃): δ (ppm) 7.09 (m, 4H), 7.28 (m, 4H). Compound 133C. LC-MS (ESI) m/z: 349 [M+H]⁺. Compound 133. LC-MS (ESI) m/z: 511 [M+H]⁺. ¹H-NMR (400 MHz, CDCl₃): δ (ppm) 0.81 (s, 3H), 1.33 (s, 3H), 3.92 (s, 1H), 7.16-7.19 (m, 5H), 7.31-7.40 (m, 7H).

### Example 134

### Synthesis of 1,3-bis(4-chlorophenyl)-4-hydroxy-5,5-dimethyl-4-(3-(trifluoromethyl)phenyl)imidazolidin-2-one

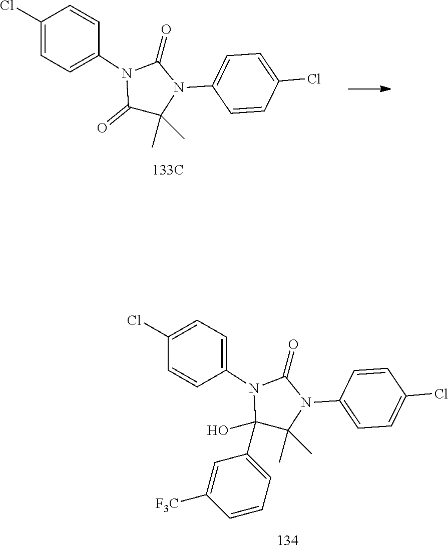 [SMILES: CC1(C)C(=O)N(c2ccc(Cl)cc2)C(=O)N1c1ccc(Cl)cc1 ; CC1(C)N(c2ccc(Cl)cc2)C(=O)N(c2ccc(Cl)cc2)C1(O)c1cccc(C(F)(F)F)c1]

Compound 134 was synthesized by employing the procedure described for Compound 127 using Compound 133C and 1-bromo-3-(trifluoromethyl)benzene in lieu of Compound 2C and 1-bromo-3-tert-butylbenzene. LC-MS (ESI) m/z: 495 [M+H]⁺. ¹H-NMR (400 MHz, CDCl₃): δ (ppm) 0.76 (s, 3H), 1.31 (s, 3H), 4.46 (s, 1H), 7.10-7.19 (m, 4H), 7.31-7.40 (m, 5H), 7.45 (s, 1H), 7.56 (d, J=7.6 Hz, 1H), 7.76 (s, 1H).

### Example 135

### Synthesis of 3-(1,3-bis(4-chlorophenyl)-4-hydroxy-2-oxo-1,3-diazaspiro[4.4]nonan-4-yl)benzonitrile

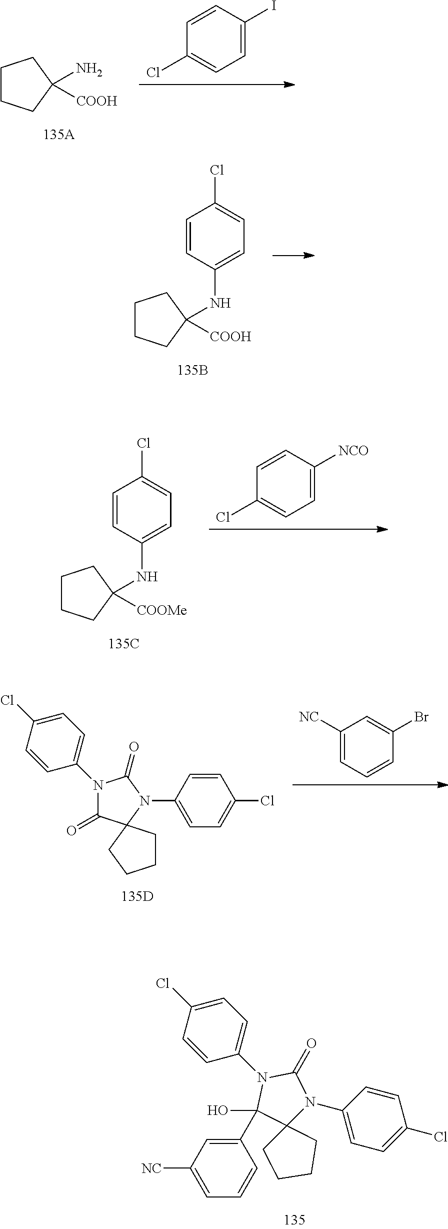

Compounds 135B, 135C, 135D, and 135 were synthesized by employing the procedures described for Compounds 224B, 224C, 224D, and 125 using Compounds 135A in the presence of 2-acetylcyclohexan-1-one, 135B, 135C, 135D, and 3-bromobenzonitrile in lieu of Compounds 224A without 2-acetylcyclohexan-1-one, 224B, 224C, 2C, and 3-bromo-N,N-dimethylaniline. Compound 135B. LC-MS (ESI) m/z: 240 [M+H]⁺. Compound 135C. LC-MS (ESI) m/z: 254 [M+H]⁺; ¹H-NMR (CDCl₃, 400 MHz): δ (ppm) 1.71-1.75 (m, 4H), 1.82-1.88 (m, 2H), 2.21-2.28 (m, 2H), 3.59 (s, 3H), 4.03 (s, 1H), 6.36 (d, J=8.8 Hz, 2H), 7.01 (d, J=8.8 Hz, 2H). Compound 135D. LC-MS (ESI) m/z: 375 [M+H]⁺. ¹H-NMR (CDCl₃, 400 MHz): δ (ppm) 1.53-1.62 (m, 2H), 1.83-1.93 (m, 2H), 2.03-2.09 (m, 2H), 2.25-2.32 (m, 2H), 7.26-7.29 (m, 2H), 7.43-7.48 (m, 6H). Compound 135. LC-MS (ESI) m/z: 478 [M+H]⁺; H-NMR (CDCl₃, 400 MHz): δ (ppm) 0.44-0.53 (m, 1H), 1.09-1.18 (m, 2H), 1.36-1.45 (m, 1H), 1.63-1.69 (m, 2H), 1.96-2.03 (m, 1H), 2.29-2.37 (m, 1H), 3.93 (s, 1H), 7.17 (d, J=9.2 Hz, 2H), 7.24 (s, 2H), 7.34 (d, J=8.8 Hz, 2H), 7.38-7.41 (m, 3H), 7.61 (d, J=8.0 Hz, 2H), 7.88 (s, 1H).

### Example 136

### Synthesis of 4-(3-acetylphenyl)-1,3-bis(4-chlorophenyl)-4-hydroxy-5-methylimidazolidin-2-one, (5S)-4-(3-acetylphenyl)-1,3-bis(4-chlorophenyl)-4-hydroxy-5-methylimidazolidin-2-one, (5R)-4-(3-acetylphenyl)-1,3-bis(4-chlorophenyl)-4-hydroxy-5-methylimidazolidin-2-one

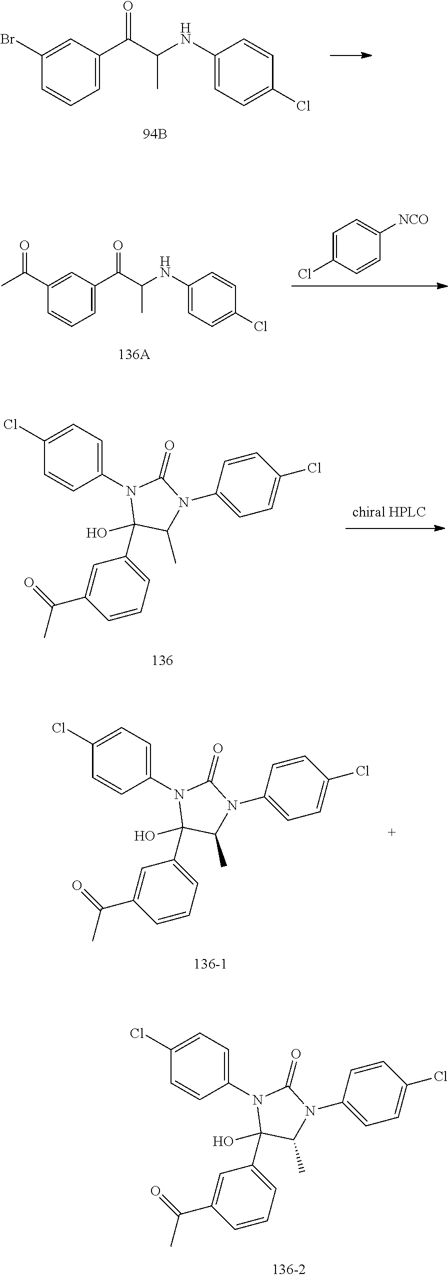

A mixture of Compound 94B (2.27 g, 6.7 mmol), tributyl(l-ethoxyvinyl)stannane (3.61 g, 10.0 mmol), and bis(triphenylphosphine)palladium(II) chloride (0.47 g, 0.67 mmol) in toluene (20 mL) was heated at 80° C. under nitrogen overnight. The reaction mixture was cooled down, diluted with hydrochloric acid (1 N, 100 mL), and extracted with ethyl acetate (50 mL×3). The combined organic layers was washed with brine (50 mL), dried over sodium sulfate, filtered, and concentrated under vacuum. The residue was purified with flash column chromatography on silica gel (ethyl acetate in petroleum ether, from 0% to 20% v/v) to furnish Compound 136A. LC-MS (ESI) m/z: 302 [M+H]⁺.

Compounds 136 was synthesized by employing the procedure described for Compound 1 using Compound 130A and 1-chloro-4-isocyanatobenzene in lieu of Compound B and 1-bromo-4-isocyanatobenzene.

Compound 136 was separated with chiral HPLC to give Compounds 136-1 and 136-2. Compound 136-1: LC-MS (ESI) m/z: 455.1 [M+H]⁺; ¹H-NMR (acetone-d₆, 400 MHz): δ (ppm) 1.29 (d, J=6.8 Hz, 3H), 2.55 (s, 3H), 4.50-4.55 (m, 1H), 6.53 (s, 1H), 7.17-7.22 (m, 2H), 7.39-7.55 (m, 7H), 7.90-7.97 (m, 2H), 8.33-8.35 (m, 1H). Chiral separation condition: MeOH contained 0.2% Methanol ammonia; IC (4.6*100 mm, 5 μm); retention time: 1.50 minutes 98%), 2.23 minutes (2%). Compound 136-2: LC-MS (ESI) m/z: 455.1 [M+H]⁺; ¹H-NMR (acetone-d₆, 400 MHz): δ (ppm) 0.79, 1.29 (d, J=6.8 Hz, 3H), 2.54, 2.55 (s, 3H), 4.50-4.55, 4.64-4.69 (m, 1H), 6.53, 6.93 (s, 1H), 7.17-7.26 (m, 2H), 7.39-7.77 (m, 7H), 7.90-7.97 (m, 2H), 8.19-8.36 (m, 1H). Chiral separation condition: MeOH contained 0.2% Methanol ammonia; IC (4.6*100 mm, 5 m); retention time: 1.960 minutes (19%), 3.28 minutes (81%).

### Example 137

### Synthesis of 1,3-bis(4-chlorophenyl)-4-hydroxy-4-(3-(1-hydroxyethyl)phenyl)-5-methylimidazolidin-2-one

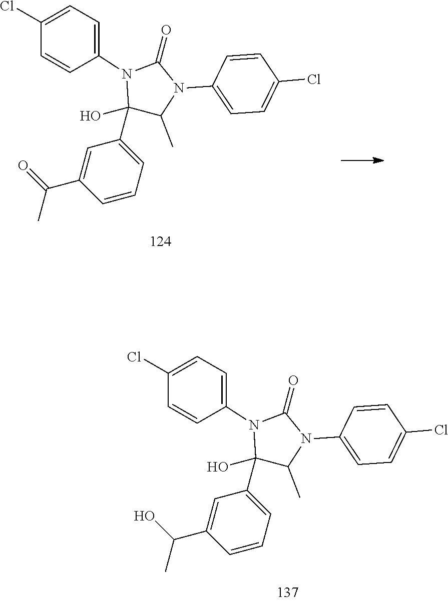 [SMILES: CC(=O)c1cccc(C2(O)C(C)N(c3ccc(Cl)cc3)C(=O)N2c2ccc(Cl)cc2)c1 ; CC(O)c1cccc(C2(O)C(C)N(c3ccc(Cl)cc3)C(=O)N2c2ccc(Cl)cc2)c1]

To a solution of Compound 124 (30 mg, 0.066 mmol) in anhydrous methanol (5 mL) at 0° C. under nitrogen atmosphere was added sodium borohydride (5 mg, 0.132 mmol). The mixture was stirred at room temperature for 3 hours, quenched by addition of acetic acid (0.2 mL) dropwise, and concentrated under reduced pressure. The residue was purified with preparative HPLC to afford Compound 137. LC-MS (ESI) m/z: 457 [M+H]⁺; ¹H-NMR (acetine-d₆, 400 MHz): δ (ppm) 0.72−0.92, 1.24-1.36 (m, 6H), 3.08-3.15, 3.28-3.34 (m, 1H), 4.40-4.48, 4.56-4.62 (m, 1H), 4.78-4.87 (m, 1H), 6.29, 6.68 (d, J=3.2 Hz, 1H), 7.14-7.78 (m, 12H).

### Example 138

### Synthesis of 3-(1,3-bis(4-chlorophenyl)-4-hydroxy-5,5-dimethyl-2-oxoimidazolidin-4-yl)benzonitrile

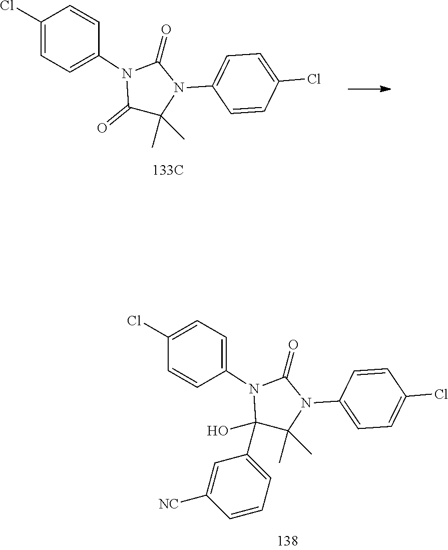 [SMILES: CC1(C)C(=O)N(c2ccc(Cl)cc2)C(=O)N1c1ccc(Cl)cc1 ; CC1(C)N(c2ccc(Cl)cc2)C(=O)N(c2ccc(Cl)cc2)C1(O)c1cccc(C#N)c1]

Compound 138 was synthesized by employing the procedure described for Compound 127 using Compound 133C and 3-bromobenzonitrile in lieu of Compound 2C and 1-bromo-3-tert-butylbenzene. LC-MS (ESI) m/z: 452 [M+H]⁺. ¹H-NMR (400 MHz, CDCl₃): δ (ppm) 0.77 (s, 3H), 1.30 (s, 3H), 4.48 (s, 1H), 7.11-7.19 (m, 4H), 7.32-7.40 (m, 5H), 7.52 (s, 1H), 7.59 (d, J=7.6 Hz, 1H), 7.78 (s, 1H).

### Example 139

### Synthesis of 5-ethyl-1,3-bis(4-fluorophenyl)-4-hydroxy-4-(3-(trifluoromethyl)phenyl)imidazolidin-2-one, (5S)-5-ethyl-1,3-bis(4-fluorophenyl)-4-hydroxy-4-(3-(trifluoromethyl)phenyl)imidazolidin-2-one, and (5R)-5-ethyl-1,3-bis(4-fluorophenyl)-4-hydroxy-4-(3-(trifluoromethyl)phenyl)imidazolidin-2-one

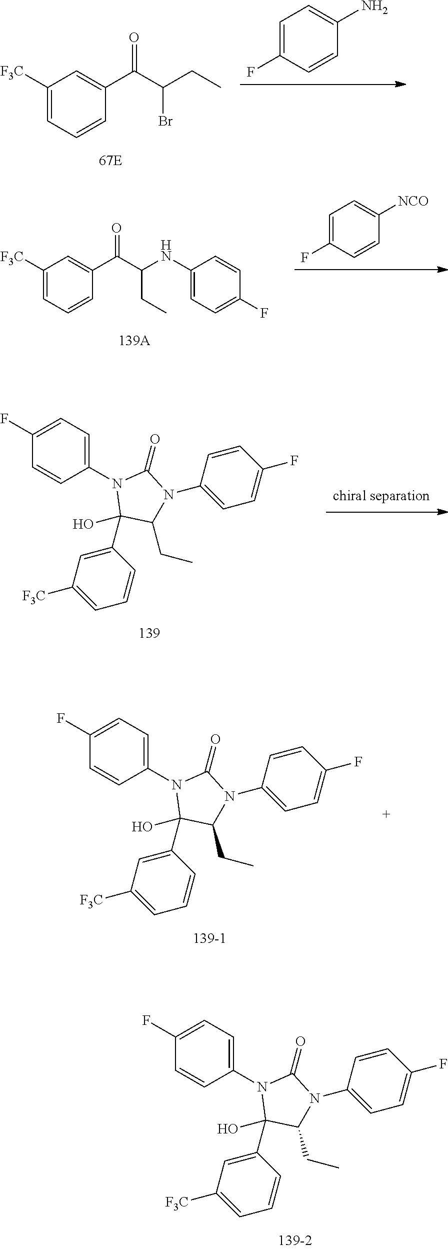

To a mixture of 4-fluoroaniline (1.98 g, 17.8 mmol) and NaHCO₃ (2.99 g, 35.6 mmol) in NMP (80 mL) was added Compound 67E (4.71 g, 16.0 mmol). The reaction mixture was stirred at 50° C. overnight, diluted with water (50 mL), and extracted with ethyl acetate (100 mL×2). The combined organic layers was washed with brine (100 mL), dried over anhydrous sodium sulfate, filtered, and concentrated under reduced pressure. The residue was purified with flash column chromatography on silica gel (ethyl acetate in petroleum ether, from 0% to 8% v/v) to yield Compound 139A. LC-MS (ESI) m/z: 326 [M+H]⁺; ¹H-NMR (CDCl₃, 400 MHz): δ (ppm) 0.90 (t, J=7.6 Hz, 3H), 1.69-1.76 (m, 1H), 2.03-2.12 (m, 1H), 4.52-4.55 (m, 1H), 4.96-4.99 (m, 1H), 6.61-6.65 (m, 2H), 6.85-6.91 (m, 2H), 7.65 (t, J=7.6 Hz, 1H), 7.87 (d, J=8.0 Hz, 1H), 8.16 (d, J=8.0 Hz, 1H), 8.23 (s, 1H).

Compound 139 was synthesized by employing the procedure described for Compound 1 using Compound 139A, 1-fluoro-4-isocyanatobenzene, and using NaHCO₃ as a base in lieu of Compound 1B, 1-bromo-4-isocyanatobenzene, and without using a base. Compound 139. LC-MS (ESI) m/z: 463 [M+H]⁺; ¹H-NMR (Acetone-d₆, 400 MHz): δ (ppm) 0.67 (t, J=7.6 Hz, 3H), 1.80-1.95 (m, 2H), 4.49-4.52 (m, 1H), 6.61 (s, 1H), 6.91-6.95 (m, 2H), 7.15-7.20 (m, 2H), 7.39-7.64 (m, 6H), 8.08-8.14 (m, 2H).

Compound 139 was separated with chiral HPLC to give Compounds 139-1 and 139-2. Compound 139-1: LC-MS (ESI) m/z: 463 [M+H]⁺; ¹H-NMR (Acetone-d₆, 400 MHz): δ (ppm) 0.68 (t, J=6.0 Hz, 3H), 1.80-1.95 (m, 2H), 4.49-4.52 (m, 1H), 6.91-6.95 (m, 2H), 6.59 (s, 1H), 7.15-7.20 (m, 2H), 7.39-7.48 (m, 4H), 7.57-7.63 (m, 2H), 8.08-8.14 (m, 2H). Chiral separation condition: MeOH contained 0.2% Methanol Ammonia; OD-H (4.6*100 mm, 5 μm); retention time: 0.86 minutes. Compound 139-2: LC-MS (ESI) m/z: 463 [M+H]⁺; ¹H-NMR (Acetone-d₆, 400 MHz): δ (ppm) 0.68 (t, J=6.0 Hz, 3H), 1.80-1.95 (m, 2H), 4.49-4.52 (m, 1H), 6.91-6.95 (m, 2H), 6.59 (s, 1H), 7.15-7.19 (m, 2H), 7.40-7.47 (m, 4H), 7.57-7.63 (m, 2H), 8.08-8.14 (m, 2H). Chiral separation condition: MeOH contained 0.2% Methanol Ammonia; OD-H (4.6*100 mm, 5 μm); retention time: 2.06 minutes.

### Example 140

### Synthesis of 1,3-bis(4-chlorophenyl)-4-hydroxy-4-(3-(2-hydroxypropan-2-yl)phenyl)-5-methylimidazolidin-2-one, (5S)-1,3-bis(4-chlorophenyl)-4-hydroxy-4-(3-(2-hydroxypropan-2-yl)phenyl)-5-methylimidazolidin-2-one, and (5R)-1,3-bis(4-chlorophenyl)-4-hydroxy-4-(3-(2-hydroxypropan-2-yl)phenyl)-5-methylimidazolidin-2-one

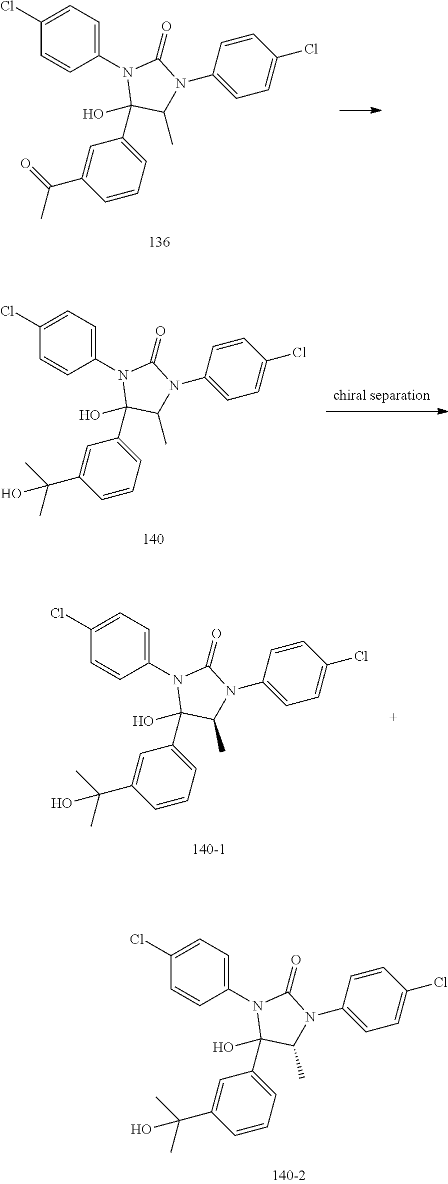 [SMILES: CC(=O)c1cccc(C2(O)C(C)N(c3ccc(Cl)cc3)C(=O)N2c2ccc(Cl)cc2)c1 ; CC1N(c2ccc(Cl)cc2)C(=O)N(c2ccc(Cl)cc2)C1(O)c1cccc(C(C)(C)O)c1 ; C[C@@H]1N(c2ccc(Cl)cc2)C(=O)N(c2ccc(Cl)cc2)C1(O)c1cccc(C(C)(C)O)c1 ; C[C@H]1N(c2ccc(Cl)cc2)C(=O)N(c2ccc(Cl)cc2)C1(O)c1cccc(C(C)(C)O)c1]

To a solution of Compound 136 (345 mg, 0.76 mmol) in dry THF (10 mL) was added a solution of methylmagnesium bromide in THF (3.0 M, 1.27 mL, 3.80 mmol) at 0° C. under nitrogen atmosphere. The mixture was stirred at 0° C. for 2 hours, quenched with saturated aqueous NH₄Cl solution (50 mL), and extracted with ethyl acetate (50 mL×3). The combined organic layers was washed with brine (50 mL), dried over anhydrous sodium sulfate, filtered, and concentrated under reduced pressure. The residue was purified with preparative HPLC to furnish Compound 140. LC-MS (ESI) m/z: 471 [M+H]⁺; ¹H-NMR (acetone-d₆, 400 MHz): δ (ppm) 0.76, 1.28 (d, J=6.8 Hz, 3H), 1.44 (d, J=8.0 Hz, 6H), 4.04 (s, 1H), 4.42-4.48, 4.57-4.62 (m, 1H), 6.28, 6.67 (s, 1H), 7.15-7.30 (m, 4H), 7.38-7.54 (m, 7H), 7.84-8.87 (m, 1H).

Compound 140 was separated with chiral HPLC to give Compounds 140-1 and 140-2. Compound 140-1: LC-MS (ESI) m/z: 471 [M+H]⁺; ¹H-NMR (acetone-d₆, 400 MHz): δ (ppm) 0.76, 1.28 (d, J=6.8 Hz, 3H), 1.44 (d, J=8.0 Hz, 6H), 4.06 (s, 1H), 4.42-4.48, 4.57-4.62 (m, 1H), 6.30, 6.69 (s, 1H), 7.15-7.30 (m, 4H), 7.38-7.54 (m, 7H), 7.84-7.87 (m, 1H). Chiral separation condition: MeOH contained 0.2% Methanol ammonia; OZ-H (4.6*250 mm, 5 μm); retention time: 2.13 minutes (87%), 2.84 minutes (13%). Compound 140-2: LC-MS (ESI) m/z: 471 [M+H]⁺; ¹H-NMR (acetone-d₆, 400 MHz): δ (ppm) 0.76, 1.28 (d, J=6.8 Hz, 3H), 1.44 (d, J=8.0 Hz, 6H), 4.06 (s, 1H), 4.42-4.48, 4.57-4.62 (m, 1H), 6.30, 6.69 (s, 1H), 7.15-7.30 (m, 4H), 7.38-7.54 (m, 7H), 7.84-7.87 (m, 1H). Chiral separation condition: MeOH contained 0.2% Methanol ammonia; OZ-H (4.6*250 mm, 5 μm); retention time: 3.04 minutes (11%), 3.99 minutes (89%).

### Example 141

### Synthesis of 1,3-bis(4-bromophenyl)-5-hydroxy-5-phenylimidazolidine-2,4-dione, (R)-1,3-bis(4-bromophenyl)-5-hydroxy-5-phenylimidazolidine-2,4-dione, and (S)-1,3-bis(4-bromophenyl)-5-hydroxy-5-phenylimidazolidine-2,4-dione

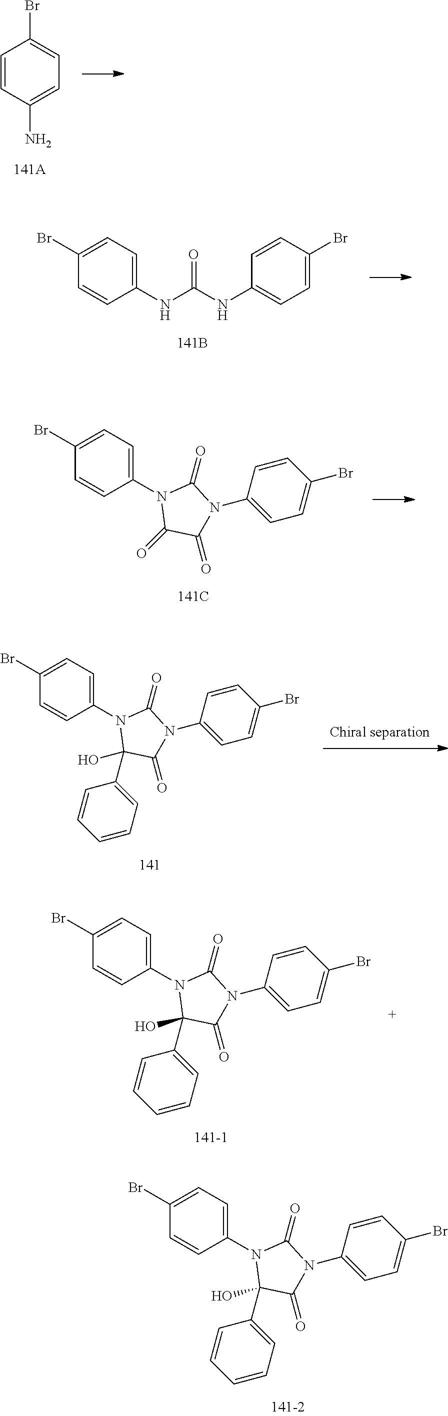 [SMILES: Nc1ccc(Br)cc1 ; O=C(Nc1ccc(Br)cc1)Nc1ccc(Br)cc1 ; O=C1C(=O)N(c2ccc(Br)cc2)C(=O)N1c1ccc(Br)cc1 ; O=C1N(c2ccc(Br)cc2)C(=O)C(O)(c2ccccc2)N1c1ccc(Br)cc1 ; O=C1N(c2ccc(Br)cc2)C(=O)[C@](O)(c2ccccc2)N1c1ccc(Br)cc1 ; O=C1N(c2ccc(Br)cc2)C(=O)[C@@](O)(c2ccccc2)N1c1ccc(Br)cc1]

To a stirred solution of Compound 141A (2 g, 11.62 mmol) and triethylamine (1.17 g, 11.62 mmol) in dichloromethane (30 mL) was added bis(trichloromethyl)carbonate (576 mg, 1.94 mmol) at 0° C. After the mixture was stirred at 0° C. for 2 hours, it was poured into water (100 mL). The precipitate was collected by filtration to furnish Compound 141B. LC-MS (ESI) m/z: 369 [M+H]⁺.

To a solution of Compound 141B (700 mg, 1.9 mmol) in dichloromethane (30 mL) was dropped oxalyl chloride (0.2 mL, 2.2 mmol). The mixture was heated at reflux overnight, washed with brine (50 mL), dried over anhydrous sodium sulfate, filtered, and concentrated. The residue was crystallized from dichloromethane-hexane to furnish Compound 141C. LC-MS (ESI) m/z: 446 [M+Na]⁺; ¹H-NMR (DMSO-d₆, 400 MHz): δ (ppm), 7.42 (dd, J=2.0, 6.4 Hz, 4H), 7.80 (dd, J=2.0, 6.4 Hz, 4H).

To a solution of Compound 141C (300 mg, 0.71 mmol) in dry THF (10 mL) was dropped a solution of phenylmagnesium bromide in THF (1 M, 1 mL, 1.0 mmol) at −78° over 5 minutes. The mixture was stirred at −78° for 1 hour. The reaction mixture was poured into saturated aqueous ammonium chloride solution (50 mL) and extracted with ethyl acetate (50 mL×3). The combined organic layers was washed with brine (100 mL), dried over anhydrous sodium sulfate, filtered, and concentrated. The crude product was purified with flash column chromatography on silica gel (ethyl acetate in petroleum, 20% v/v) to furnish Compound 141. LC-MS (ESI) m/z: 501 [M+H]⁺; ¹H-NMR (CDCl₃, 400 MHz): δ (ppm), 4.45 (s, 1H), 7.33-7.40 (m, 9H), 7.45 (m, 2H), 7.59-7.61 (m, 2H).

Compound 141 was separated with chiral HPLC to furnish Compound 141-1 and Compound 141-2. Compound 141-1: LC-MS (ESI) m/z: 501 [M+H]⁺; ¹H-NMR (CD₃OD, 400 MHz): δ (ppm), 7.37-7.51 (m, 9H), 7.58-7.60 (m, 2H), 7.69-7.71 (m, 2H). Chiral separation condition: MeOH, (R,R)-Whelk-Ol (4.6*250 mm 5 μm); rentention time: 4.36 minutes. Compound 141-2: LC-MS (ESI) m/z: 501 [M+H]⁺; ¹H-NMR (CD₃OD, 400 MHz)): δ (ppm), 7.37-7.51 (m, 9H), 7.58-7.60 (m, 2H), 7.69-7.71 (m, 2H). Chiral separation condition: MeOH, (R,R)-Whelk-Ol (4.6*250 mm 5 μm); rentention time: 6.75 minutes.

### Example 142

### Synthesis of 5-hydroxy-1,3,5-triphenylimidazolidine-2,4-dione

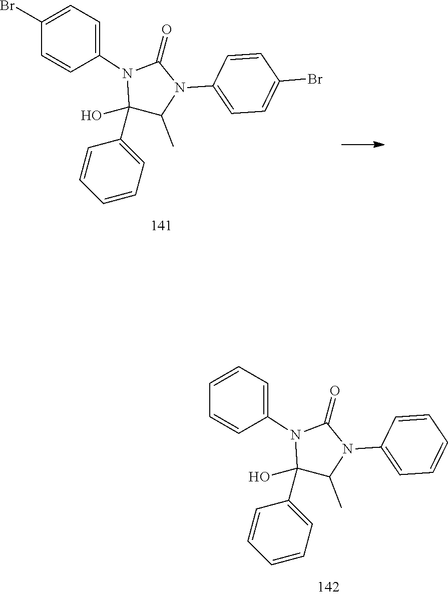 [SMILES: CC1N(c2ccc(Br)cc2)C(=O)N(c2ccc(Br)cc2)C1(O)c1ccccc1 ; CC1N(c2ccccc2)C(=O)N(c2ccccc2)C1(O)c1ccccc1]

A suspension of Compound 141 (70 mg, 0.139 mmol) and Pd/C (30 mg) in methanol (30 mL) was stirred under hydrogen at room temperature overnight. After removal of Pd/C by filtration, the filtrate was concentrated to furnish Compound 142. LC-MS (ESI) m/z: 345 [M+H]⁺; ¹H-NMR (CD₃OD, 400 MHz: δ (ppm), 7.03-7.07 (m, 1H), 7.12-7.16 (m, 2H), 7.21-7.28 (m, 3H), 7.32-7.35 (m, 1H), 7.38-7.43 (m, 6H), 7.47-7.50 (m, 2H).

### Example 143

### Synthesis of 1,3-bis(4-bromophenyl)-5-ethyl-5-hydroxyimidazolidine-2,4-dione

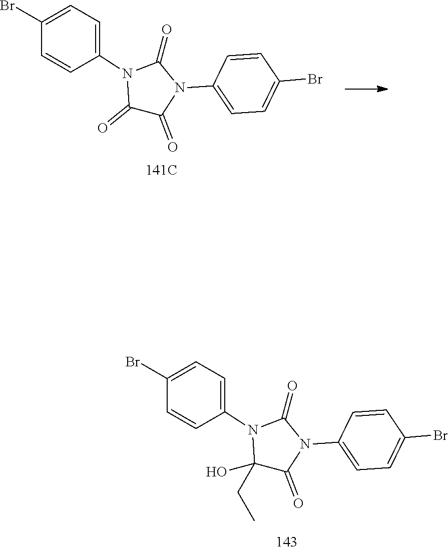 [SMILES: O=C1C(=O)N(c2ccc(Br)cc2)C(=O)N1c1ccc(Br)cc1 ; CCC1(O)C(=O)N(c2ccc(Br)cc2)C(=O)N1c1ccc(Br)cc1]

Compound 143 was synthesized by employing the procedure described for Compound 141 using ethylmagnesium bromide in lieu of phenylmagnesium bromide. LC-MS (ESI) m/z: 453 [M+H]⁺; ¹H-NMR (CD₃OD, 400 MHz): δ (ppm) 0.84-0.88 (m, 3H), 1.84-2.10 (m, 2H), 7.40-7.42 (m, 2H), 7.58-7.64 (m, 4H), 7.68-7.70 (m, 2H).

### Example 144

### Synthesis of 1,3-bis(4-bromophenyl)-5-hydroxy-5-(4-methoxyphenyl)imidazolidine-2,4-dione

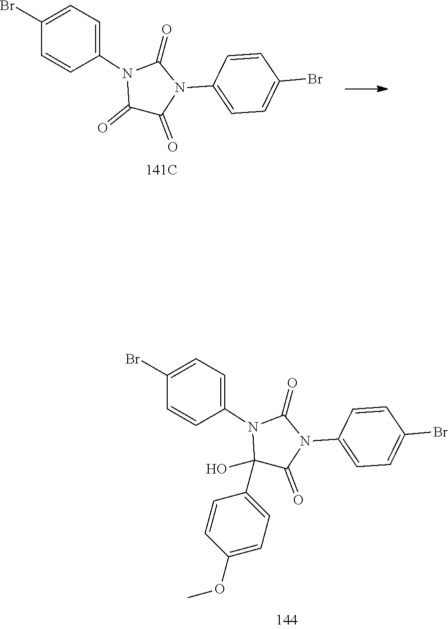 [SMILES: O=C1C(=O)N(c2ccc(Br)cc2)C(=O)N1c1ccc(Br)cc1 ; COc1ccc(C2(O)C(=O)N(c3ccc(Br)cc3)C(=O)N2c2ccc(Br)cc2)cc1]

Compound 144 was synthesized by employing the procedure described for Compound 141 using (4-methoxyphenyl)magnesium bromide in lieu of phenylmagnesium bromide. LC-MS (ESI) m/z: 531 [M+H]⁺; ¹H-NMR (CDCl₃, 400 MHz): δ (ppm) 3.79 (s, 3H), 6.85-6.87 (m, 2H), 7.23-7.37 (m, 8H), 7.53-7.55 (m, 2H).

### Example 145

### Synthesis of 5-hydroxy-1,3-bis(4-isopropylphenyl)-5-phenylimidazolidine-2,4-dione

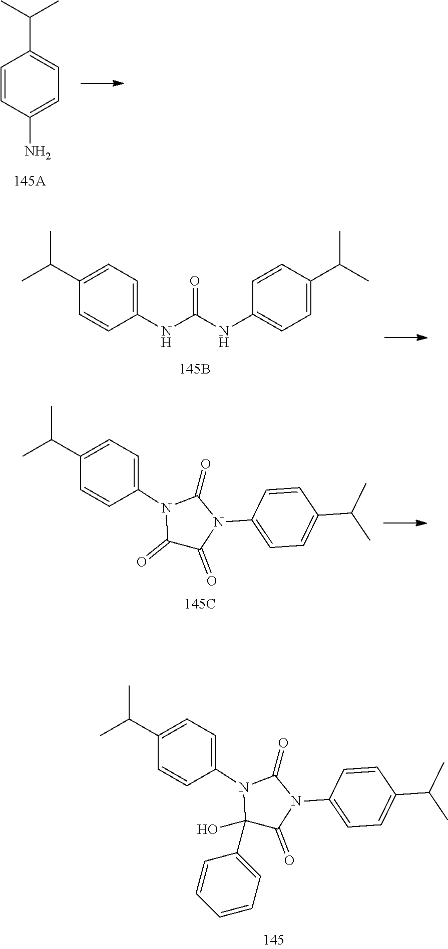

Compounds 145B, 145C, and 145 were synthesized by employing the procedures described for Compounds 141B, 141C, and 141 using 145A, 145B, and 145C in lieu of 141A, 141B, and 141C. Compound 145B. LC-MS (ESI) m/z: 297 [M+H]⁺. Compound 145C. LC-MS (ESI) m/z: 351 [M+H]⁺; ¹H-NMR (DMSO-d₆, 400 MHz): δ (ppm) 1.23-1.25 (m, 12H), 2.93-2.99 (m, 2H), 7.36-7.44 (m, 8H). Compound 145. LC-MS (ESI) m/z: 429 [M+H]⁺; ¹H-NMR (DMSO-d₆, 400 MHz): δ (ppm) 1.13-1.14 (m, 6H), 1.23-1.25 (m, 6H), 2.77-2.84 (m, 1H), 2.93-2.99 (m, 1H), 7.15-7.17 (m, 2H), 7.34-7.43 (m, 9H), 7.57-7.59 (m, 2H), 8.21 (s, 1H).

### Example 146

### Synthesis of 1,3-bis(4-chlorophenyl)-4-hydroxy-4-(3-(trifluoromethoxy)phenyl)-1,3-diazaspiro[4.4]nonan-2-one

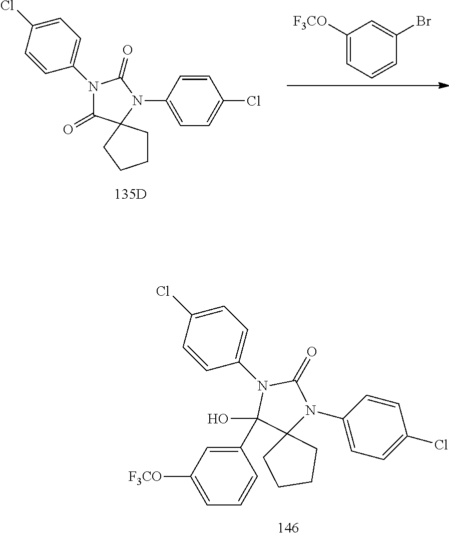 [SMILES: O=C1N(c2ccc(Cl)cc2)C(=O)C2(CCCC2)N1c1ccc(Cl)cc1 ; FC(F)(F)Oc1cccc(Br)c1 ; O=C1N(c2ccc(Cl)cc2)C2(CCCC2)C(O)(c2cccc(OC(F)(F)F)c2)N1c1ccc(Cl)cc1]

Compound 146 was synthesized by employing the procedure described for Compound 125 using Compound 135D and 1-bromo-3-(trifluoromethoxy)benzene in lieu of Compound 2C and 3-bromo-N,N-dimethylaniline. LC-MS (ESI) m/z: 537 [M+H]⁺; H-NMR (CDCl₃, 400 MHz): δ (ppm) 0.45-0.52 (m, 1H), 1.09-1.16 (m, 2H), 1.37-1.43 (m, 1H), 1.63-1.64 (m, 2H), 1.91-1.99 (m, 1H), 2.32-2.39 (m, 1H), 3.91 (s, 1H), 7.15-7.17 (m, 4H), 7.20-7.23 (m, 2H), 7.30-7.41 (m, 6H).

### Example 147

### Synthesis of 1,3-bis(4-chlorophenyl)-5-hydroxy-5-phenylimidazolidine-2,4-dione

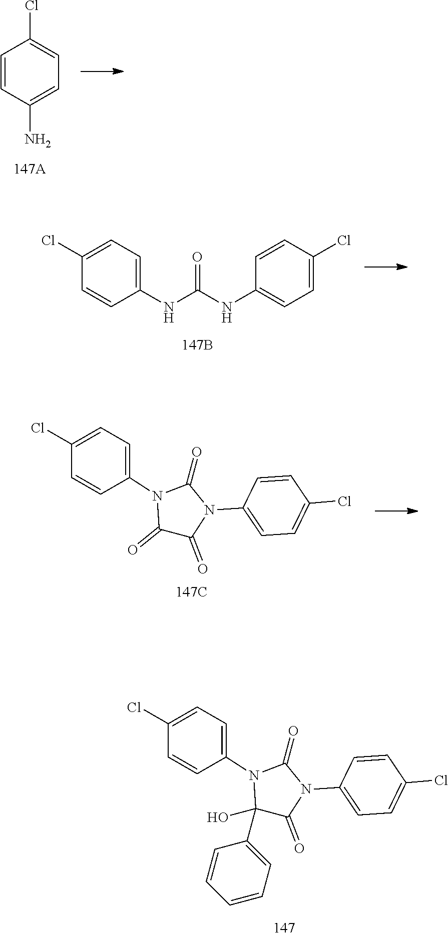 [SMILES: Nc1ccc(Cl)cc1 ; O=C(Nc1ccc(Cl)cc1)Nc1ccc(Cl)cc1 ; O=C1C(=O)N(c2ccc(Cl)cc2)C(=O)N1c1ccc(Cl)cc1 ; O=C1N(c2ccc(Cl)cc2)C(=O)C(O)(c2ccccc2)N1c1ccc(Cl)cc1]

Compounds 147B, 147C, and 147 were synthesized by employing the procedures described for Compounds 141B, 141C, and 141 using 147A, 147B, and 147C in lieu of 141A, 141B, and 141C. Compound 147B. LC-MS (ESI) m/z: 281 [M+H]⁺. Compound 147C. LC-MS (ESI) m/z: 357 [M+Na]⁺. Compound 147. LC-MS (ESI) m/z: 413 [M+H]⁺; ¹H-NMR (CDCl₃, 400 MHz): δ (ppm) 4.72 (s, 1H), 7.20-7.22 (m, 2H), 7.35-7.41 (m, 7H), 7.42-7.46 (m, 4H).

### Example 148

### Synthesis of 1,3-bis(4-bromophenyl)-5-hydroxy-5-(naphthalen-2-yl)imidazolidine-2,4-dione

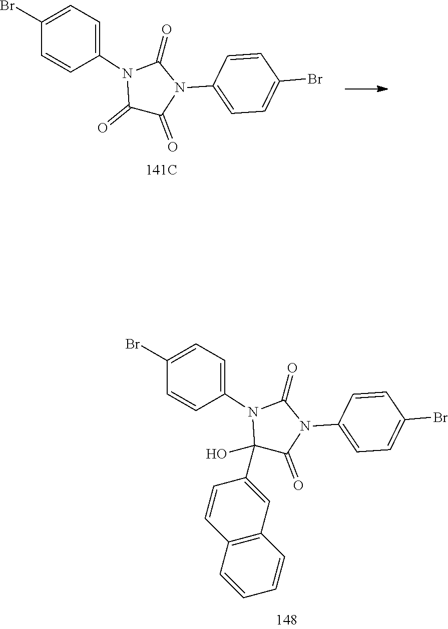 [SMILES: O=C1C(=O)N(c2ccc(Br)cc2)C(=O)N1c1ccc(Br)cc1 ; O=C1N(c2ccc(Br)cc2)C(=O)C(O)(c2ccc3ccccc3c2)N1c1ccc(Br)cc1]

Compound 148 was synthesized by employing the procedure described for Compound 141 using naphthalen-2-ylmagnesium bromide in lieu of phenylmagnesium bromide. LC-MS (ESI) m/z: 551 [M+H]⁺; ¹H-NMR (CDCl₃, 400 MHz): δ (ppm) 4.55 (brs, 1H), 7.32-7.40 (m, 7H), 7.54-7.62 (m, 4H), 7.84-7.86 (m, 3H), 8.06-8.07 (m, 1H).

### Example 149

### Synthesis of 4-(3-chlorophenyl)-1,3-bis(4-chlorophenyl)-5-hydroxy-4-methylimidazolidin-2-one, (4S)-4-(3-chlorophenyl)-1,3-bis(4-chlorophenyl)-5-hydroxy-4-methylimidazolidin-2-one, and (4R)-4-(3-chlorophenyl)-1,3-bis(4-chlorophenyl)-5-hydroxy-4-methylimidazolidin-2-one

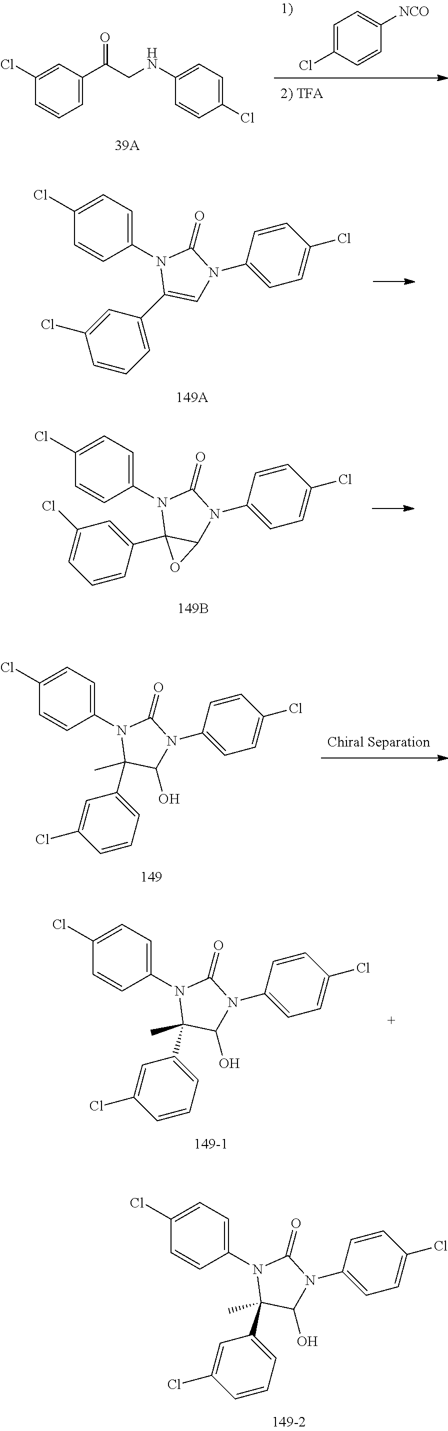

Compounds 149A, 149B, and 149 were synthesized by employing the procedures described for Compounds 86A, 86B, and 86 using Compounds (39A, 1-chloro-4-isothiocyanatobenzene, 149A, and 149B in lieu of Compounds 1B, 1-bromo-4-isothiocyanatobenzene, 86A, and 86B. Compound 149A. LC-MS (ESI) m/z: 415 [M+H]⁺.

Compound 149B. LC-MS (ESI) m/z: 431 [M+H]⁺; ¹H-NMR (CDCl₃, 400 MHz): δ (ppm) 5.55 (s, 1H), 7.25-7.47 (m, 12H). Compound 149. LC-MS (ESI) m/z: 447 [M+H]⁺; ¹H-NMR (CDCl₃, 400 MHz): δ (ppm) 1.01 (s, 3H), 3.34 (s, 1H), 5.07 (s, 1H), 7.18-7.20 (m, 3H), 7.31-7.44 (m, 9H).

Compound 149 was separated with chiral HPLC to yield Compound 149-1 and Compound 149-2. Compound 149-1: LC-MS (ESI) m/z: 447 [M+H]⁺; ¹H-NMR (CDCl₃, 400 MHz): δ (ppm) 0.94 (s, 3H), 4.12 (s, 1H), 5.00 (s, 1H), 7.13-7.28 (m, 4H), 7.29-7.44 (m, 8H). Chiral separation condition: MeOH contained 0.2% Methanol Ammonia; OJ-H (250*4.6 mm, 5 μm); retention time: 3.87 minutes. Compound 149-1: LC-MS (ESI) m/z: 447 [M+H]⁺; ¹H-NMR (CDCl₃, 400 MHz): δ (ppm) 0.92 (s, 3H), 4.98 (s, 1H), 7.09-7.26 (m, 4H), 7.27-7.37 (m, 8H). Chiral separation condition: MeOH contained 0.2% Methanol Ammonia; OJ-H (250*4.6 mm, 5 μm); retention time: 4.84 minutes.

### Example 150

### Synthesis of 5,7-bis(4-chlorophenyl)-8-hydroxy-8-(3-(trifluoromethoxy)phenyl)-2-oxa-5,7-diazaspiro[3.4]octan-6-one

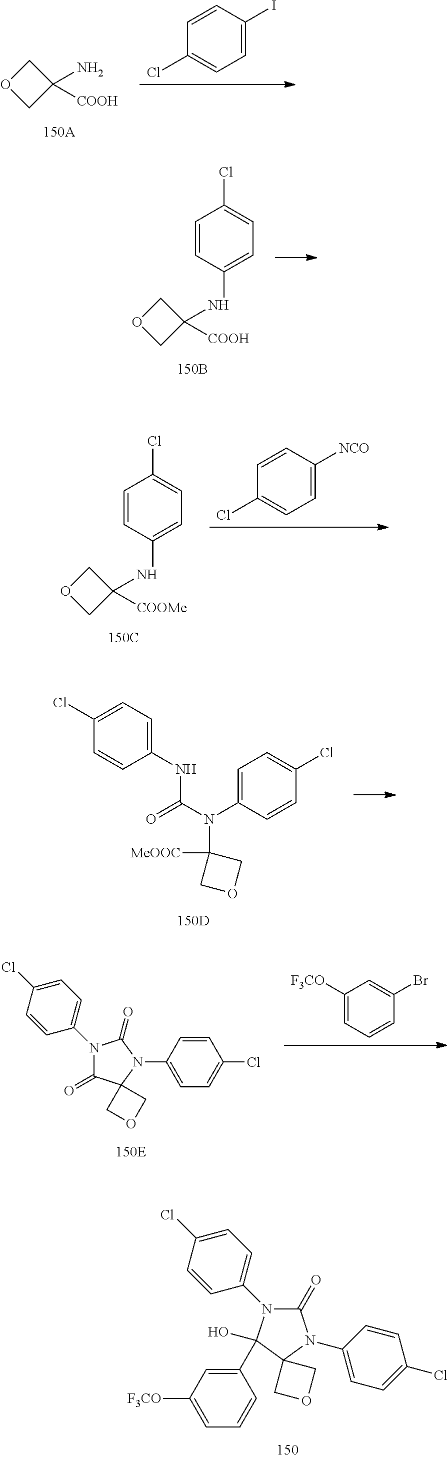

Compounds 150B, 150C, 150D, 150E, and 150 were synthesized by employing the procedures described for Compounds 224B, 224C, 224D, 224E, and 125 using Compounds 150A in the presence of 2-acetylcyclohexan-1-one, 150B, 150C, 150D, 150E, and 1-bromo-3-(trifluoromethoxy)benzene in lieu of Compounds 224A without 2-acetylcyclohexan-1-one, 224B, 224C, 224D, 2C, and 3-bromo-N,N-dimethylaniline. Compound 150B. LC-MS (ESI) m/z: 228 [M+H]⁺. Compound 150C. LC-MS (ESI) m/z: 242 [M+H]⁺. Compound 150D. LC-MS (ESI) m/z: 395 [M+H]⁺. Compound 150E. LC-MS (ESI) m/z: 363 [M+H]⁺; ¹H-NMR (CDCl₃, 400 MHz): δ (ppm) 4.91 (d, J=7.6 Hz, 2H), 5.15 (d, J=7.6 Hz, 2H), 7.45-7.55 (m, 8H). Compound 150. LC-MS (ESI) m/z: 525 [M+H]⁺; ¹H-NMR (CDCl₃, 400 MHz): δ (ppm) 3.96 (d, J=8.0 Hz, 1H), 4.38 (d, J=8.0 Hz, 1H), 4.57 (d, J=8.4 Hz, 1H), 5.01 (s, 1H), 5.12 (d, J=8.8 Hz, 1H), 7.11-7.22 (m, 7H), 7.30-7.42 (m, 5H).

### Example 151

### Synthesis of 3-(5,7-bis(4-chlorophenyl)-8-hydroxy-6-oxo-2-oxa-5,7-diazaspiro[3.4]octan-8-yl)benzonitrile

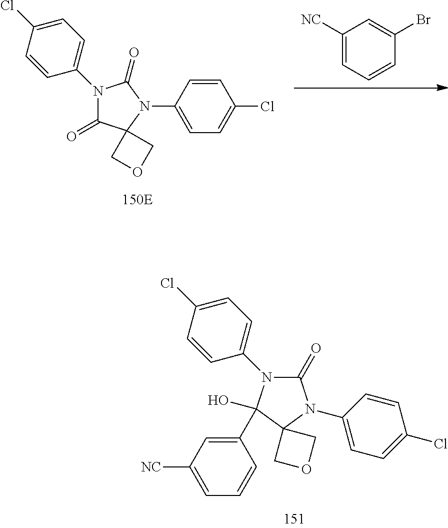 [SMILES: O=C1N(c2ccc(Cl)cc2)C(=O)C2(COC2)N1c1ccc(Cl)cc1 ; N#Cc1cccc(Br)c1 ; N#Cc1cccc(C2(O)N(c3ccc(Cl)cc3)C(=O)N(c3ccc(Cl)cc3)C23COC3)c1]

Compound 151 was synthesized by employing the procedure described for Compound 125 using Compound 150E and 3-bromobenzonitrile in lieu of Compound 2C and 3-bromo-N,N-dimethylaniline. LC-MS (ESI) m/z: 466 [M+H]⁺; ¹H-NMR (CDCl₃, 400 MHz): δ (ppm) 3.87 (d, J=7.6 Hz, 1H), 4.41 (d, J=8.4 Hz, 1H), 4.63 (d, J=8.4 Hz, 1H), 5.05 (s, 1H), 5.12 (d, J=8.4 Hz, 1H), 7.14-7.20 (m, 6H), 7.37 (d, J=8.8 Hz, 2H), 7.45-7.55 (m, 2H), 7.64 (d, J=8.4 Hz, 1H), 7.84 (s, 1H).

### Example 152

### Synthesis of 1,3-bis(4-bromophenyl)-5-butyl-5-hydroxyimidazolidine-2,4-dione

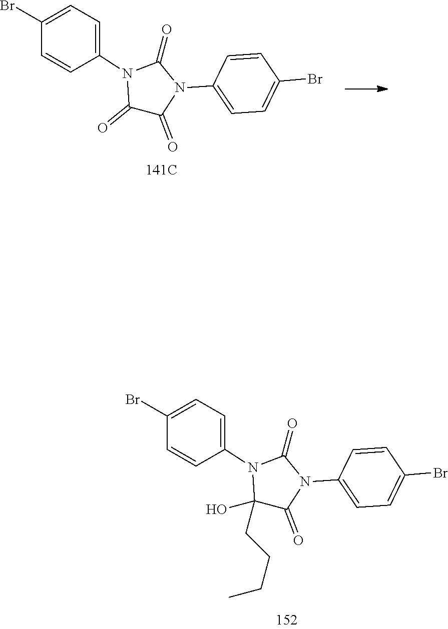 [SMILES: O=C1C(=O)N(c2ccc(Br)cc2)C(=O)N1c1ccc(Br)cc1 ; CCCCC1(O)C(=O)N(c2ccc(Br)cc2)C(=O)N1c1ccc(Br)cc1]

Compound 152 was synthesized by employing the procedure described for Compound 141 using n-BuLi in lieu of phenylmagnesium bromide. LC-MS (ESI) m/z: 481 [M+H]⁺; ¹H-NMR (CD₃OD, 400 MHz): δ (ppm) 0.80-0.83 (m, 3H), 1.19-1.27 (m, 4H), 1.80-1.87 (m, 1H), 1.98-2.04 (m, 1H), 7.38-7.40 (m, 2H), 7.56-7.63 (m, 4H), 7.67-7.69 (m, 2H).

### Example 153

### Synthesis of 5-hydroxy-5-(4-isopropylphenyl)-1,3-diphenylimidazolidine-2,4-dione

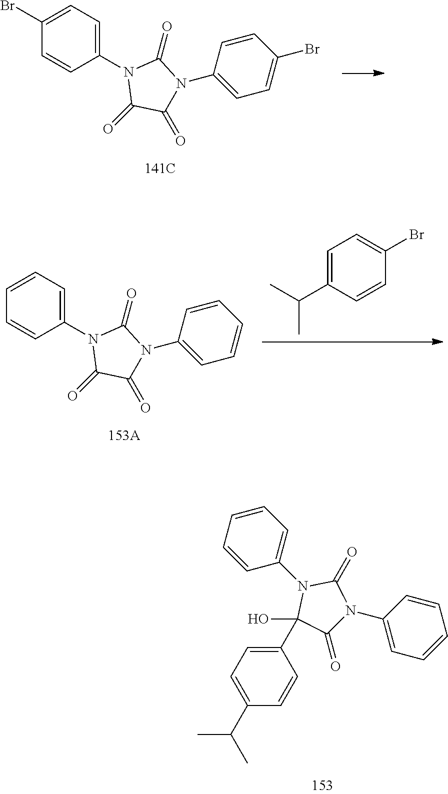

Compounds 153A and 153 were synthesized by employing the procedures described for Compounds 142 and 127 using 141C, 153A, and 1-bromo-4-isopropylbenzene in lieu of 141, 2C, and 1-bromo-4-(tert-butyl)benzene. Compound 153A. LC-MS (m/z): 267 [M+H]⁺. Compound 153. LC-MS (ESI) m/z: 387 [M+H]⁺; ¹H-NMR (CDCl₃, 400 MHz): δ (ppm) 1.20 (d, J=7.2 Hz, 6H), 2.82-2.89 (m, 1H), 4.57 (brs, 1H), 7.15-7.27 (m, 5H), 7.38-7.45 (m, 9H).

### Example 154

### Synthesis of 1,3,5-tris(4-chlorophenyl)-5-hydroxyimidazolidine-2,4-dione

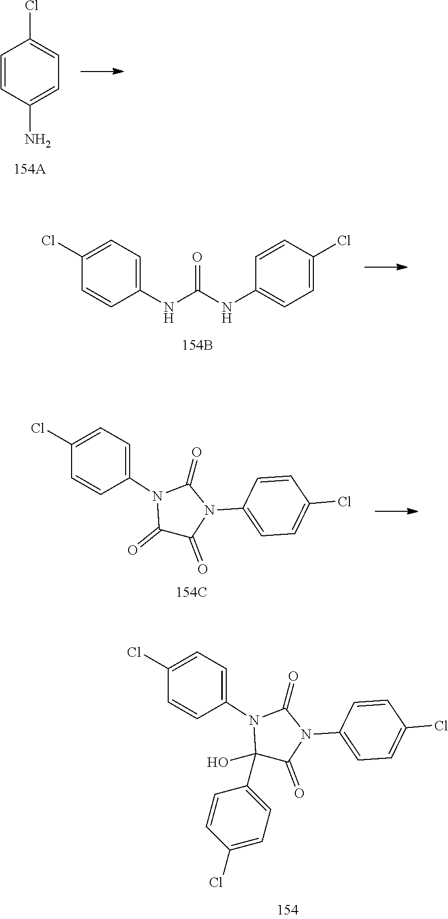 [SMILES: Nc1ccc(Cl)cc1 ; O=C(Nc1ccc(Cl)cc1)Nc1ccc(Cl)cc1 ; O=C1C(=O)N(c2ccc(Cl)cc2)C(=O)N1c1ccc(Cl)cc1 ; O=C1N(c2ccc(Cl)cc2)C(=O)C(O)(c2ccc(Cl)cc2)N1c1ccc(Cl)cc1]

Compounds 154B, 154C, and 154 were synthesized by employing the procedures described for Compounds 141B, 141C, and 141 using 154A, 154B, and 154C in lieu of 141A, 141B, and 141C. Compound 154B. LC-MS (ESI) m/z: 281 [M+H]⁺. Compound 154C. LC-MS (ESI) m/z: 357 [M+Na]⁺. Compound 154. LC-MS (ESI) m/z: 447 [M+H]⁺; ¹H-NMR (CDCl₃, 400 MHz): δ (ppm), 7.20-7.22 (m, 2H), 7.24-7.26 (m, 2H), 7.28-7.38 (m, 8H).

### Example 155

### Synthesis of 1,3-bis(4-chloro-2-fluorophenyl)-5-hydroxy-5-phenylimidazolidine-2,4-dione

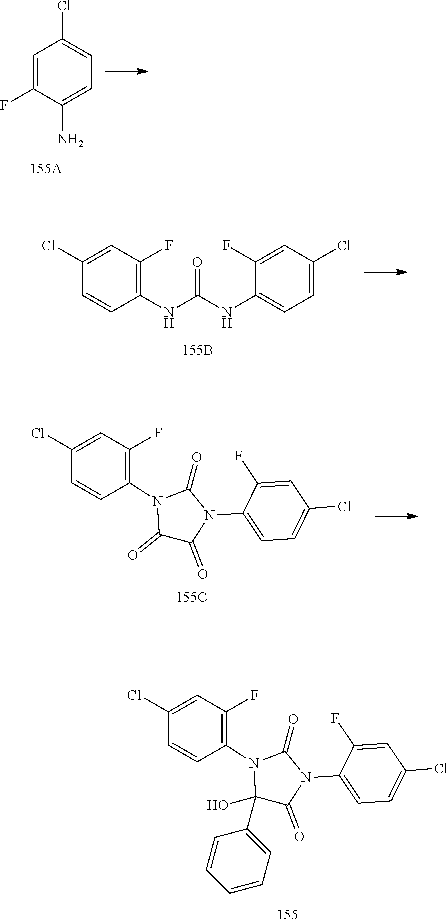 [SMILES: Nc1ccc(Cl)cc1F ; O=C(Nc1ccc(Cl)cc1F)Nc1ccc(Cl)cc1F ; O=C1C(=O)N(c2ccc(Cl)cc2F)C(=O)N1c1ccc(Cl)cc1F ; O=C1N(c2ccc(Cl)cc2F)C(=O)C(O)(c2ccccc2)N1c1ccc(Cl)cc1F]

Compounds 155B, 155C, and 155 were synthesized by employing the procedures described for Compounds 141B, 141C, and 141 using 155A, 155B, and 155C in lieu of 141A, 141B, and 141C. Compound 155B. LC-MS (ESI) m/z: 316.8 [M+H]⁺. Compound 155C. LC-MS (ESI) m/z: 393 [M+Na]⁺. Compound 155. LC-MS (ESI) m/z: 449.0 [M+H]⁺; ¹H-NMR (CDCl₃, 400 MHz): δ (ppm), 7.01-7.03 (m, 1H), 7.07-7.10 (m, 2H), 7.24-7.25 (m, 1H), 7.29-7.39 (m, 5H), 7.44-7.46 (m, 2H).

### Example 156

### Synthesis of 5-(4-cyclopropoxyphenyl)-5-hydroxy-1,3-diphenylimidazolidine-2,4-dione

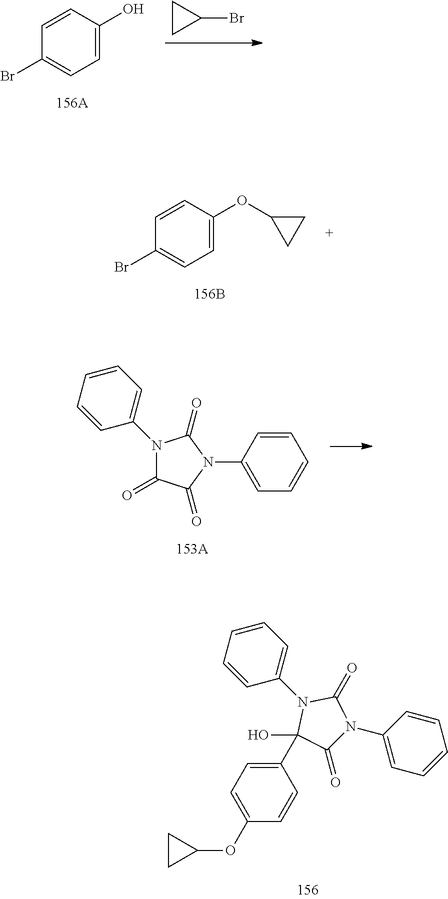

To a solution of Compound 156A (3 g, 17.4 mmol) in NMP (100 mL) was added bromocyclopropane (6.28 g, 52 mmol) and Cs₂CO₃ (11.34 g, 35 mmol). The mixture was stirred at 145° C.-155° C. overnight, treated with water (100 mL), extracted with ethyl acetate (100 mL×3). The combined extracts was washed with water (100 mL×3) and brine (100 mL), dried over anhydrous sodium sulfate, filtered, and concentrated. The residue was purified with flash column chromatography on silica gel (petroleum ether, 100%, v) to furnish Compound 156B. LC-MS (ESI) m/z: non-ionizable compound under routine conditions used; ¹H-NMR: (CDCl₃, 400 MHz): δ (ppm) 0.74-0.77 (m, 4H), 3.66-3.71 (m, 1H), 6.91-6.93 (d, J=8.8 Hz, 2H), 7.36 (d, J=8.8 Hz, 2H).

Compound 156 was synthesized by employing the procedure described for Compound 127 using 156B and 153A in lieu of 1-bromo-4-(tert-butyl)benzene and 2C. LC-MS (ESI) m/z: 401 [M+H]⁺; ¹H-NMR (CDCl₃, 400 MHz): δ (ppm) 0.75-0.77 (m, 4H), 3.69-3.72 (m, 1H), 7.03 (d, J=8.8 Hz, 2H), 7.18-7.22 (m, 1H), 7.26-7.31 (m, 2H), 7.41-7.44 (m, 3H), 7.47-7.49 (m, 6H).

### Example 157

### Synthesis of 1,3-bis(4-bromophenyl)-5-hexyl-5-hydroxyimidazolidine-2,4-dione

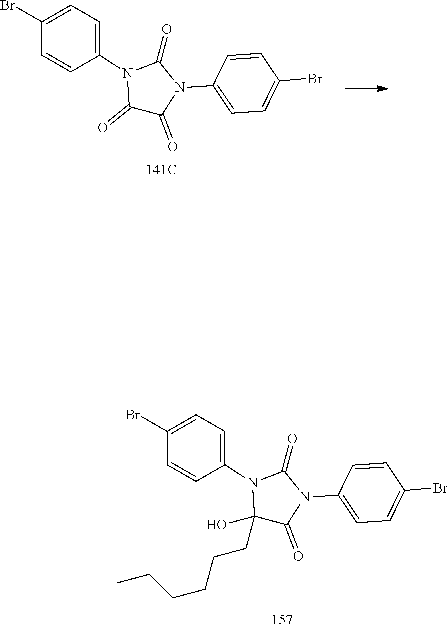 [SMILES: O=C1C(=O)N(c2ccc(Br)cc2)C(=O)N1c1ccc(Br)cc1 ; CCCCCCC1(O)C(=O)N(c2ccc(Br)cc2)C(=O)N1c1ccc(Br)cc1]

Compound 157 was synthesized by employing the procedure described for Compound 141 using hexylmagnesium bromide in lieu of phenylmagnesium bromide. LC-MS (ESI) m/z: 1039 [2M+Na]⁺; ¹H-NMR (CDCl₃, 400 MHz): δ (ppm) 0.82 (t, J=6.8, 3H), 1.12-1.20 (m, 8H), 1.78-2.03 (m, 2H), 7.23 (d, J=8.8, 2H), 7.41 (d, J=8.8, 2H), 7.51-7.56 (m, 4H).

### Example 158

### Synthesis of 3-(1,3-bis(4-chlorophenyl)-4-hydroxy-5,5-dimethyl-2-oxoimidazolidin-4-yl)-5-bromobenzonitrile

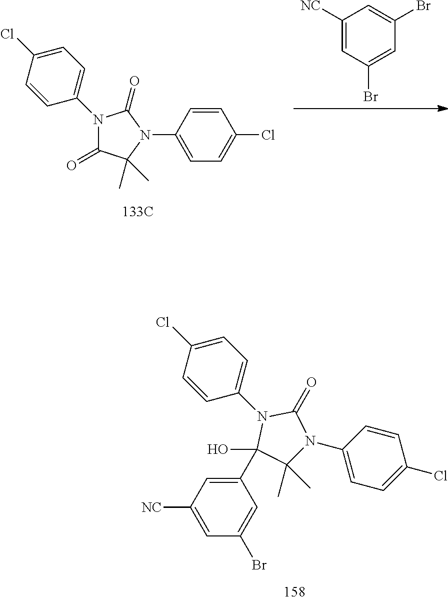 [SMILES: CC1(C)C(=O)N(c2ccc(Cl)cc2)C(=O)N1c1ccc(Cl)cc1 ; N#Cc1cc(Br)cc(Br)c1 ; CC1(C)N(c2ccc(Cl)cc2)C(=O)N(c2ccc(Cl)cc2)C1(O)c1cc(Br)cc(C#N)c1]

Compound 158 was synthesized by employing the procedure described for Compound 125 using Compound 133C and 3,5-dibromobenzonitrile in lieu of Compound 2C and 3-bromo-N,N-dimethylaniline. LC-MS (ESI) m/z: 530 [M+H]⁺; ¹H-NMR (CDCl₃, 400 MHz): δ (ppm) 0.79 (s, 3H), 1.29 (s, 3H), 4.62 (s, 1H), 7.08 (d, J=8.8 Hz, 2H), 7.19 (d, J=8.4 Hz, 2H), 7.30-7.35 (m, 4H), 7.59 (s, 1H), 7.71 (s, 1H), 7.80 (s, 1H).

### Example 159

### Synthesis of 1,3-bis(4-chlorophenyl)-4-hydroxy-5-methyl-4-(3-(trifluoromethoxy)phenyl)-5-(trifluoromethyl)imidazolidin-2-one, (5S)-1,3-bis(4-chlorophenyl)-4-hydroxy-5-methyl-4-(3-(trifluoromethoxy)phenyl)-5-(trifluoromethyl)imidazolidin-2-one, and (5R)-1,3-bis(4-chlorophenyl)-4-hydroxy-5-methyl-4-(3-(trifluoromethoxy)phenyl)-5-(trifluoromethyl)imidazolidin-2-one

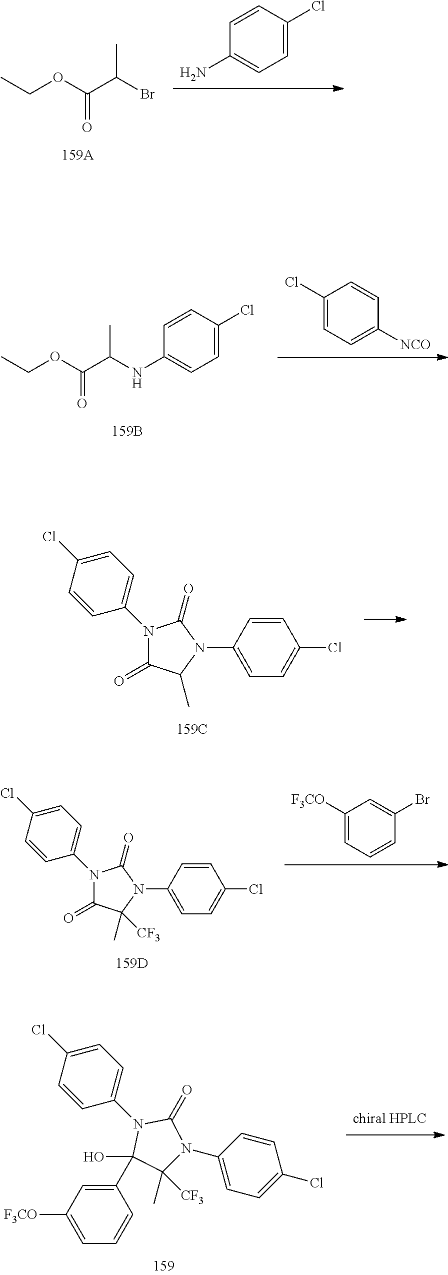

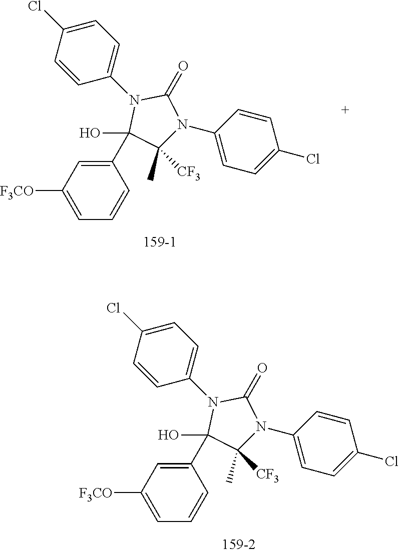 [SMILES: C[C@]1(C(F)(F)F)N(c2ccc(Cl)cc2)C(=O)N(c2ccc(Cl)cc2)C1(O)c1cccc(OC(F)(F)F)c1 ; C[C@@]1(C(F)(F)F)N(c2ccc(Cl)cc2)C(=O)N(c2ccc(Cl)cc2)C1(O)c1cccc(OC(F)(F)F)c1]

Compounds 159B and 159C were synthesized by employing the procedures described for Compounds 2B and 128C using Compounds 159A, 4-chloroaniline, and 159B in lieu of Compounds 2A, 4-bromoaniline, and 128B. Compound 159B. LC-MS (ESI) m/z: 228 [M+H]⁺; ¹H-NMR (CDCl₃, 400 MHz): δ (ppm) 1.25 (d, J=6.8 Hz, 3H), 1.46 (d, J=6.8 Hz, 3H), 4.04-4.11 (m, 1H), 4.14-4.22 (m, 3H), 6.53 (d, J=8.8 Hz, 2H), 7.12 (d, J=8.8 Hz, 2H). Compound 159C. LC-MS (ESI) m/z: 335 [M+H]⁺; ¹H-NMR (CDCl₃, 400 MHz): δ (ppm) 1.58 (d, J=7.2 Hz, 3H), 4.68-4.73 (m, 1H), 7.37-7.48 (m, 8H).

To a solution of Compound 159C (1.5 g, 5.0 mmol) in anhydrous THF (15 mL) was dropped a solution of LDA in n-hexane (1.0M, 5.5 mL, 5.5 mmol) at −78° C. under nitrogen atmosphere. After the mixture was stirred at −78° C. for 30 minutes, to it was added 5-(trifluoromethyl)-5H-dibenzo[b,d]thiophenium trifluoromethanesulfonate (2.2 g, 5.5 mmol). After stirring at −78° C. for 10 minutes, the reaction mixture was warmed to room temperature and stirred at room temperature overnight. The mixture was quenched with saturated ammonium chloride solution (20 mL) and extracted with ethyl acetate (30 mL×2). The combined extracts were washed with brine (40 mL), dried over anhydrous sodium sulfate, filtered, and evaporated under reduced pressure. The residue was purified with preparative HPLC to furnish Compound 159D: LC-MS (ESI) m/z: 403 [M+H]⁺; ¹H-NMR (CDCl₃, 400 MHz): δ (ppm) 1.81 (s, 3H), 7.25-7.27 (m, 2H), 7.41-7.48 (m, 6H).

Compound 159 was synthesized by employing the procedure described for Compound 125 using Compound 159D and 1-bromo-3-(trifluoromethoxy)benzene in lieu of Compound 2C and 3-bromo-N,N-dimethylaniline. LC-MS (ESI) m/z: 565 [M+H]⁺; ¹H-NMR (CDCl₃, 400 MHz): δ (ppm) 1.03 (s, 3H), 7.185-7.22 (m, 4H), 7.26-7.31 (m, 4H), 7.39-7.42 (m, 4H).

Compound 159 was separated by using chiral HPLC to give Compound 159-1 and Compound 159-2. Compound 159-1: LC-MS (ESI) m/z: 565 [M+H]⁺; ¹H-NMR (CDCl₃, 400 MHz): δ (ppm) 1.04 (s, 3H), 3.49 (s, 1H), 7.19-7.31 (m, 8H), 7.40-7.43 (m, 4H). Chiral separation conditions: MeOH contained 0.2% Methanol Ammonia; OJ-H (4.6*100 mm, 5 μm), retention time: 0.83 minutes. Compound 159-2: LC-MS (ESI) m/z: 565 [M+H]⁺; ¹H-NMR (CDCl₃, 400 MHz): δ (ppm) 1.04 (s, 3H), 3.47 (s, 1H), 7.14-7.31 (m, 8H), 7.40-7.52 (m, 4H). Chiral separation conditions: MeOH contained 0.2% Methanol Ammonia, OJ-H (4.6*100 mm, 5 m), retention time: 1.01 minutes.

### Example 160

### Synthesis of 5-hydroxy-1,3-bis(4-methoxyphenyl)-5-phenylimidazolidine-2,4-dione

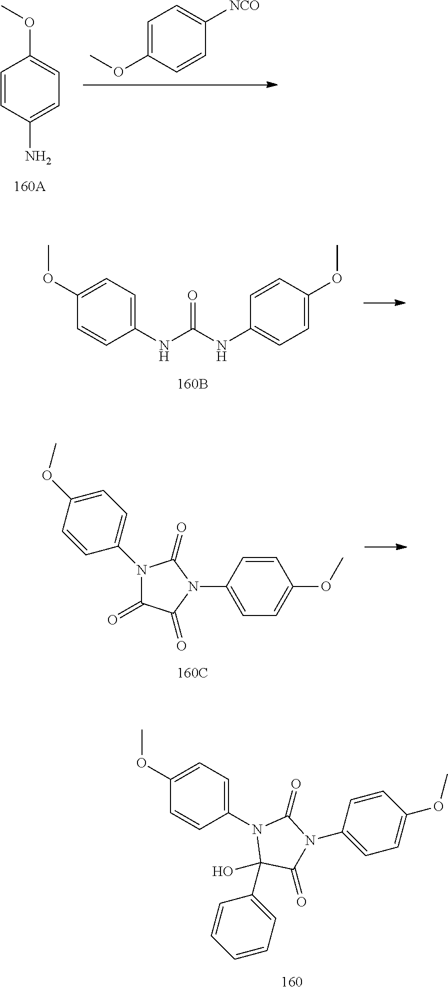

To a stirred solution of Compound 160A (1.23 g, 10 mmol) and triethylamine (1.21 g, 12 mmol) in dichloromethane (40 mL) was added to 1-isocyanato-4-methoxybenzene (1.49 g, 10 mmol) at 0° C. The mixture was stirred at 0° C. for 2 hours and poured into water (100 mL). The precipitate was collected by filtration. It was dried under vacuum to furnish Compound 160B. LC-MS (ESI) m/z: 273 [M+H]⁺.

Compounds 160C and 160 were synthesized by employing the procedures described for Compounds 141C and 141 using 160B and 160C in lieu of 141B and 141C. Compound 160C. LC-MS (ESI) m/z: 327 [M+H]⁺. Compound 160. LC-MS (ESI) m/z: 405 [M+H]⁺; ¹H-NMR (CDCl₃, 400 MHz): δ (ppm) 3.71 (s, 3H), 3.81 (s, 3H), 4.95 (s, 1H), 6.66-6.69 (m, 2H), 6.86-6.95 (m, 2H), 7.11-7.20 (m, 2H), 7.23-7.26 (m, 2H), 7.30-7.39 (m, 3H), 7.41-7.52 (m, 2H).

### Example 161

### Synthesis of 1,3-bis(4-fluorophenyl)-5-hydroxy-5-phenylimidazolidine-2,4-dione

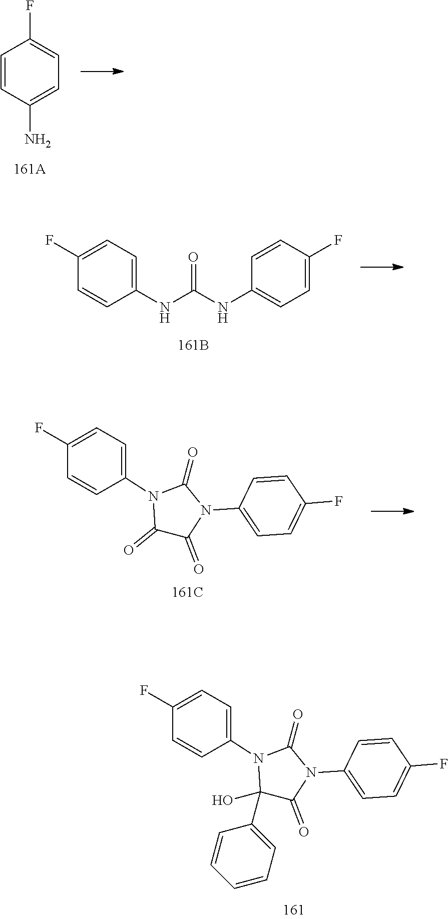 [SMILES: Nc1ccc(F)cc1 ; O=C(Nc1ccc(F)cc1)Nc1ccc(F)cc1 ; O=C1C(=O)N(c2ccc(F)cc2)C(=O)N1c1ccc(F)cc1 ; O=C1N(c2ccc(F)cc2)C(=O)C(O)(c2ccccc2)N1c1ccc(F)cc1]

Compounds 161B, 161C, and 161 were synthesized by employing the procedures described for Compounds 160B, 141C, and 141 using 161A, 1-fluoro-4-isocyanatobenzene, 161B, and 161C in lieu of 160A, 1-isocyanato-4-methoxybenzene, 141B, and 141C. Compound 161B. LC-MS (ESI) m/z: 249 [M+H]⁺. Compound 161C. LC-MS (ESI) m/z: 325 [M+Na]⁺. Compound 161. LC-MS (ESI) m/z: 381 [M+H]⁺; ¹H-NMR (CDCl₃, 400 MHz): δ (ppm), 4.66 (s, 1H), 6.86 (t, J=8.6 Hz, 2H), 7.07 (t, J=8.6 Hz, 2H), 7.21-7.42 (m, 9H).

### Example 162

### Synthesis of 1,3-bis(3-chlorophenyl)-5-hydroxy-5-phenylimidazolidine-2,4-dione

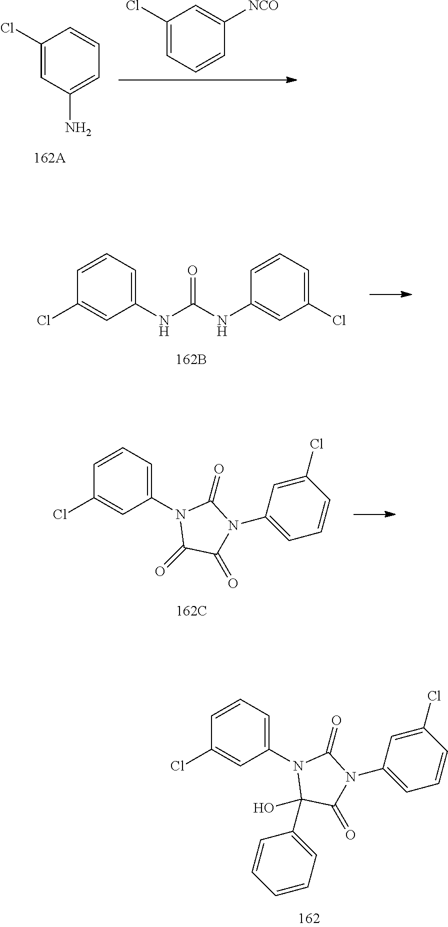

Compounds 162B, 162C, and 162 were synthesized by employing the procedures described for Compounds 160B, 141C, and 141 using 162A, 1-chloro-3-isocyanatobenzene, 162B, and 162C in lieu of 160A, 1-isocyanato-4-methoxybenzene, 141B, and 141C. Compound 162B. LC-MS (ESI) m/z: 281 [M+H]⁺. Compound 162C. LC-MS (ESI) m/z: 357 [M+Na]⁺. Compound 162. LC-MS (ESI) m/z: 413 [M+H]⁺; ¹H-NMR (CDCl₃, 400 MHz): δ (ppm) 4.43 (s, 1H), 7.06-7.15 (m, 2H), 7.23-7.37 (m, 7H), 7.37-7.47 (m, 3H), 7.51-7.52 (m, 1H).

### Example 163

### Synthesis of 1,3-bis(4-chlorophenyl)-4-ethyl-5-hydroxy-4-methyl-5-(3-(trifluoromethoxy)phenyl)imidazolidin-2-one, (4R)-1,3-bis(4-chlorophenyl)-4-ethyl-5-hydroxy-4-methyl-5-(3-(trifluoromethoxy)phenyl)imidazolidin-2-one, and (4S)-1,3-bis(4-chlorophenyl)-4-ethyl-5-hydroxy-4-methyl-5-(3-(trifluoromethoxy)phenyl)imidazolidin-2-one

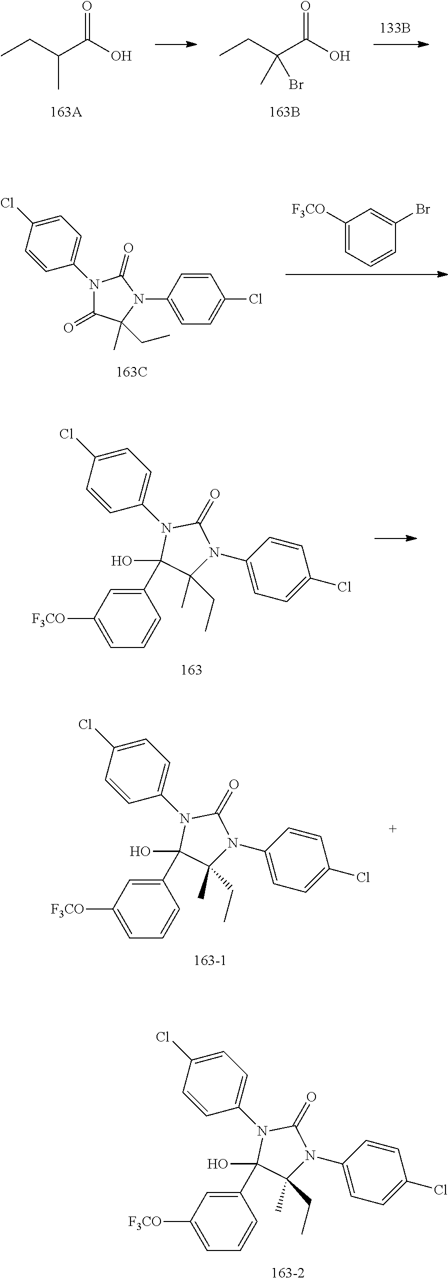

A mixture of Compound 163A (5.1 g, 50 mmol) and NBS (9.3 g, 52.5 mmol) in CCl₄ (50 mL) under nitrogen was heated to reflux overnight. The color of bromine had disappeared and the mixture was cooled down to room temperature. The resulting solid of succinimide was filtered off and the filtrate was concentrated under reduced pressure to give a crude product, which was distilled in vacuo (collected at 53° C./0.2 mmHg) to afford Compound 163B: ¹H-NMR (400 MHz, CDCl₃): δ (ppm) 1.05 (t, J=7.6 Hz, 3H), 1.90 (s, 3H), 2.15 (m, 2H).

Compounds 163C and 163 were synthesized by employing the procedures described for Compounds 120D and 125 using Compounds 163B, 133B, 163C, and 1-bromo-3-(trifluoromethoxy)benzene in lieu of 2-bromo-2-methylpropanoic acid, Compounds 120C, 2C, and 3-bromo-N,N-dimethylaniline. Compound 163C. LC-MS (ESI) m/z: 363 [M+H]⁺. Compound 163. LC-MS (ESI) m/z: 525 [M+H]⁺; ¹H-NMR (400 MHz, CDCl₃): δ (ppm) 0.86 (t, J=7.6 Hz, 3H), 0.91 (s, 3H), 1.74-1.80 (m, 1H), 1.96-2.02 (m, 1H), 3.62 (s, 1H), 7.14-7.19 (m, 5H), 7.26-7.29 (m, 2H), 7.34-7.38 (m, 5H).

Compound 163 was separated by using chiral HPLC to give Compound 163-1 and Compound 163-2. Compound 163-1: LC-MS (ESI) m/z: 525 [M+H]⁺; ¹H-NMR (400 MHz, CDCl₃): δ (ppm) 0.88 (t, J=7.6 Hz, 3H), 0.92 (s, 3H), 1.77-1.83 (m, 1H), 1.97-2.03 (m, 1H), 3.45 (s, 1H), 7.15-7.19 (m, 5H), 7.29-7.30 (m, 2H), 7.33-7.39 (m, 5H). Chiral separation conditions: MeOH contained 0.2% Methanol Ammonia; OD-H (4.6*100 mm, 5 μm); retention time: 0.74 minutes (99%). Compound 163-2: LC-MS (ESI) m/z: 525 [M+H]⁺; ¹H-NMR (400 MHz, CDCl₃): δ (ppm) 0.86 (t, J=7.6 Hz, 3H), 0.91 (s, 3H), 1.75-1.81 (m, 1H), 1.96-2.01 (m, 1H), 3.54 (s, 1H), 7.14-7.19 (m, 5H), 7.26-7.28 (m, 2H), 7.32-7.38 (m, 5H). Chiral separation conditions: MeOH contained 0.2% Methanol Ammonia; OD-H (4.6*100 mm, 5 μm); retention time: 2.74 minutes (99%).

### Example 164

### Synthesis of 1,3-bis(4-bromophenyl)-5-(4-cyclopropoxyphenyl)-5-hydroxyimidazolidine-2,4-dione

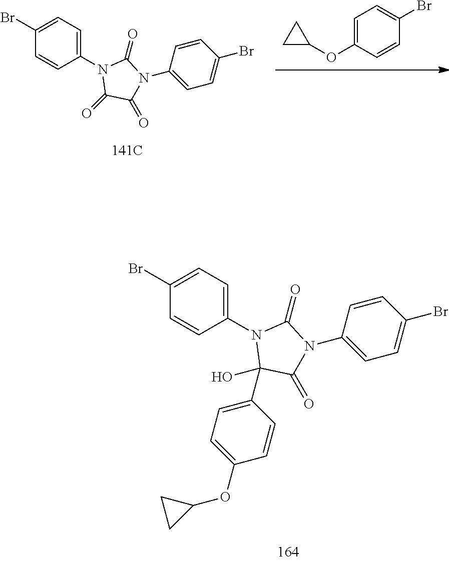

Compound 164 was synthesized by employing the procedure described for Compound 127 using 141C and 1-bromo-4-cyclopropoxybenzene in lieu of 2C and 1-bromo-4-(tert-butyl)benzene. LC-MS (ESI) m/z: non-ionizable compound under routine conditions used; ¹H-NMR (CDCl₃, 400 MHz): δ (ppm) 0.75-0.78 (m, 4H), 3.69-3.73 (m, 1H), 7.02 (d, J=8.8 Hz, 2H), 7.31-7.41 (m, 8H), 7.59 (d, J=8.4 Hz, 2H).

### Example 165

### Synthesis of 5,7-bis(4-chlorophenyl)-8-(3-(difluoromethoxy)phenyl)-8-hydroxy-5,7-diazaspiro[3.4]octan-6-one

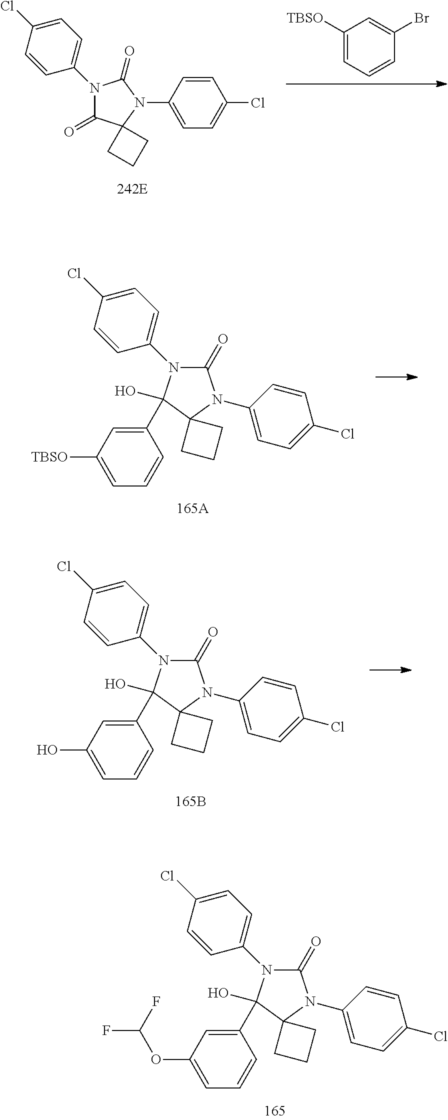 [SMILES: O=C1N(c2ccc(Cl)cc2)C(=O)C2(CCC2)N1c1ccc(Cl)cc1 ; CC(C)(C)[Si](C)(C)Oc1cccc(Br)c1 ; CC(C)(C)[Si](C)(C)Oc1cccc(C2(O)N(c3ccc(Cl)cc3)C(=O)N(c3ccc(Cl)cc3)C23CCC3)c1 ; O=C1N(c2ccc(Cl)cc2)C2(CCC2)C(O)(c2cccc(O)c2)N1c1ccc(Cl)cc1 ; O=C1N(c2ccc(Cl)cc2)C2(CCC2)C(O)(c2cccc(OC(F)F)c2)N1c1ccc(Cl)cc1]

Compounds 165A, 165B, and 165 were synthesized by employing the procedures described for Compounds 125, 227, and 236 using (3-bromophenoxy)(tert-butyl)dimethylsilane, Compounds 242E, 165A, and 165B in lieu of 3-bromo-N,N-dimethylaniline, Compounds 2C, 227A, and 227. Compound 165A. LC-MS (ESI) m/z: 569 [M+H]⁺. Compound 165B. LC-MS (ESI) m/z: 455 [M+H]⁺; ¹H-NMR (400 MHz, CDCl₃): δ (ppm) 0.88-0.97 (m, 2H), 1.66-1.69 (m, 1H), 1.90-1.93 (m, 1H), 2.09-2.12 (m, 1H), 2.67-2.71 (m, 1H), 6.66-6.69 (m, 1H), 6.76-6.88 (m, 2H), 7.01-7.14 (m, 1H), 7.25-7.27 (m, 3H), 7.47-7.51 (m, 4H), 7.57-7.60 (m, 2H), 9.44 (s, 1H). Compound 165. LC-MS (ESI) m/z: 505 [M+H]⁺; ¹H-NMR (400 MHz, CDCl₃): δ (ppm) 0.86-0.89 (m, 1H), 1.07-1.10 (m, 1H), 1.75-1.78 (m, 1H), 1.96-1.99 (m, 1H), 2.19-2.23 (m, 1H), 2.69-2.71 (m, 1H), 3.99 (s, 1H), 6.26-6.63 (m, 1H), 7.06-7.09 (m, 1H), 7.13-7.19 (m, 4H), 7.30-7.39 (m, 7H).

### Example 166

### Synthesis of 1,3-bis(4-chlorophenyl)-5-((dimethylamino)methyl)-4-hydroxy-4-(3-(trifluoromethoxy)phenyl)imidazolidin-2-one, (5S)-1,3-bis(4-chlorophenyl)-5-((dimethylamino)methyl)-4-hydroxy-4-(3-(trifluoromethoxy)phenyl)imidazolidin-2-one, and (5R)-1,3-bis(4-chlorophenyl)-5-((dimethylamino)methyl)-4-hydroxy-4-(3-(trifluoromethoxy)phenyl)imidazolidin-2-one

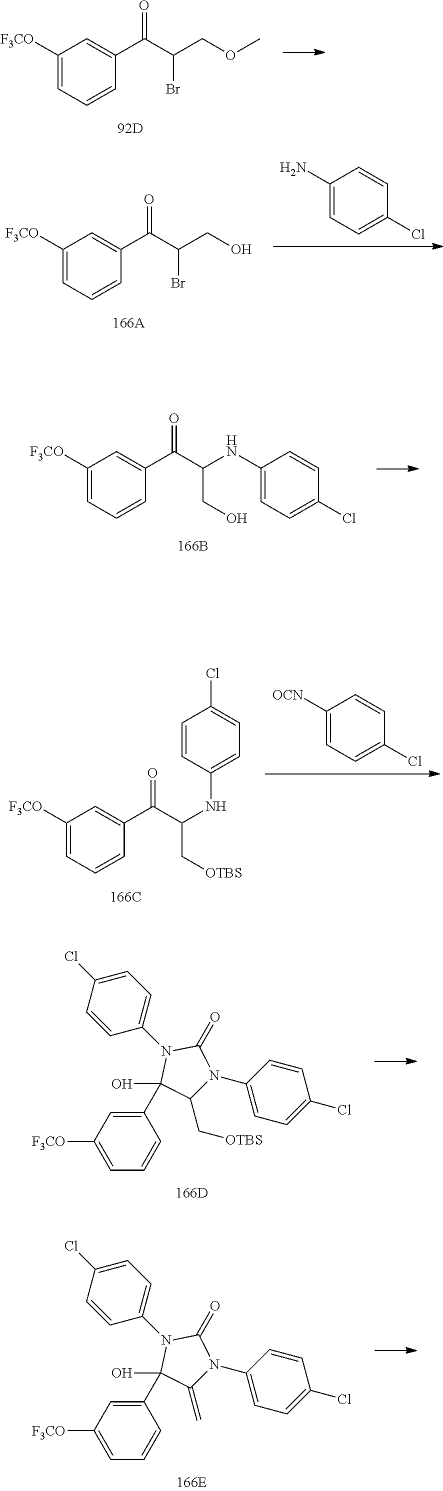

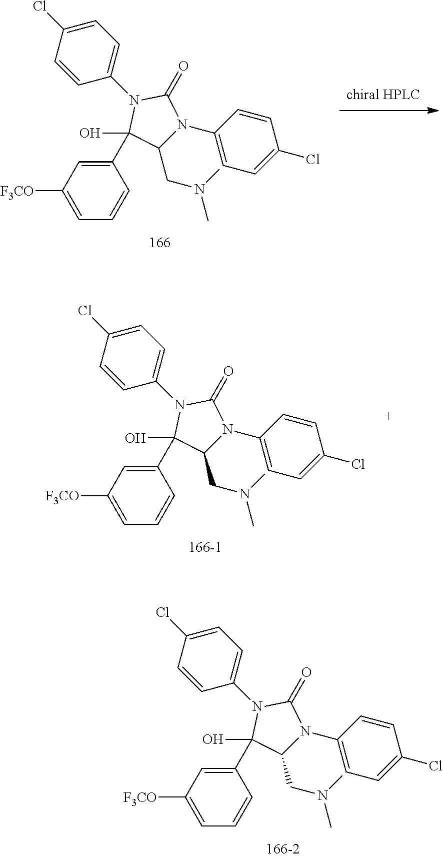 [SMILES: CN(C)CC1N(c2ccc(Cl)cc2)C(=O)N(c2ccc(Cl)cc2)C1(O)c1cccc(OC(F)(F)F)c1 ; CN(C)C[C@@H]1N(c2ccc(Cl)cc2)C(=O)N(c2ccc(Cl)cc2)C1(O)c1cccc(OC(F)(F)F)c1 ; CN(C)C[C@H]1N(c2ccc(Cl)cc2)C(=O)N(c2ccc(Cl)cc2)C1(O)c1cccc(OC(F)(F)F)c1]

To a stirred solution of Compound 92D (3.2 g, 10 mmol) in DCM (40 mL) was dropped a solution of boron tribromide in methylene chloride (1 M, 20 mL, 20 mmol) at 0° C. and stirred at 25° C. for 1 hour. The mixture was poured into ice water (200 mL) and extracted with DCM (150 mL). The extract was washed with water (200 mL) and brine (200 mL), dried over anhydrous sulfate sodium, filtered, and concentrated. The residue was purified with flash column chromatographyh on silica gel (ethyl acteate in petroleum ether, from 0% to 10% v/v) to afford Compound 166A: LC-MS (ESI) m/z: 313 [M+H]⁺.

Compound 166B was synthesized by employing the procedure described for Compound 1B using Compound 166A and 4-chloroaniline using NMP as solvent at 55° C. in lieu of Compound 1A and 4-bromoaniline using EtOH as solvent at 25° C. LC-MS (ESI) m/z: 360 [M+H]⁺.

To a solution of Compound 166B (1 g, 2.78 mmol) in DCM (20 mL) was added imidazole (284 mg, 4.17 mmol) and TBSCl (504 mg, 3.34 mmol) and the mixture was stirred at 25° C. overnight. The mixture was concentrated and purified with flash column chromatographyh on silica gel (ethyl acteate in petroleum ether, from 0% to 10% v/v) to yield Compound 166C: LC-MS (ESI) m/z: 474 [M+H]⁺.

Compound 166D was synthesized by employing the procedure described for Compound 1 using Compound 166C and 1-chloro-4-isocyanatobenzene in lieu of Compound 1B and 1-bromo-4-isocyanatobenzene. LC-MS (ESI) m/z: 627 [M+H]⁺.

To a solution of Compound 166D (150 mg, 0.3 mmol) in THF (10 mL) was added TBAF (70 mg, 0.23 mmol), and the mixture was stirred at room temperature for 2 hours. The reaction mixture was evaporated under vacuum, the residue was purified with flash column chromatography on silica gel (ethyl acetate in petroleum ether, 20% v/v) to furnish Compound 166E: LC-MS (ESI) m/z: 495 [M+H]⁺.

A mixture of Compound 166E (80 mg, 0.16 mmol) and a solution of dimethylamine in THF (2 M, 5 mL) was heated at 100° C. for 2 hours in a microwave oven. The mixture was evaporated under reduced pressure. The residue was purify with preparative HPLC to furnish Compound 166: LC-MS (ESI) m/z: 540[M+H]⁺. ¹H-NMR (CDCl₃, 400 MHz): δ (ppm): 2.29 (s, 6H), 2.79-2.84 (m, 1H), 2.98-3.02 (m, 1H), 4.17 (t, J=3.6 Hz, 1H), 7.15-7.18 (m, 3H), 7.32-7.44 (m, 8H), 7.51 (m, 1H).

Compound 166 was separated by using chiral HPLC to give Compound 166-1 and Compound 166-2. Compound 166-1: LC-MS (ESI) m/z: 540 [M+H]⁺; ¹H-NMR (CDCl₃, 400 MHz): δ (ppm) 2.30 (s, 6H), 2.79-2.84 (m, 1H), 2.98-3.02 (m, 1H), 4.17 (t, J=3.6 Hz, 1H), 7.16-7.18 (m, 3H), 7.32-7.45 (m, 8H), 7.51 (s, 1H), 10.92 (s, 1H). Chiral separation conditions: MeOH contained 0.2% Methanol Ammonia; EnantioPak OD (100*4.6 mm, 5 μm); retention time: 1.02 minutes. Compound 166-2: LC-MS (ESI) m/z: 540 [M+H]⁺; ¹H-NMR (CDCl₃, 400 MHz): δ (ppm) 2.30 (s, 6H), 2.79-2.84 (m, 1H), 2.98-3.02 (m, 1H), 4.17 (t, J=3.2 Hz, 1H), 7.16-7.18 (m, 3H), 7.32-7.44 (m, 8H), 7.51 (s, 1H), 10.95 (s, 1H). Chiral separation conditions: MeOH contained 0.2% Methanol Ammonia; EnantioPak OD (100*4.6 mm, 5 μm); retention time: 1.58 minutes.

### Example 167

### Synthesis of 1,3-bis(4-bromophenyl)-5-(4-(cyclopentyloxy)phenyl)-5-hydroxyimidazolidine-2,4-dione

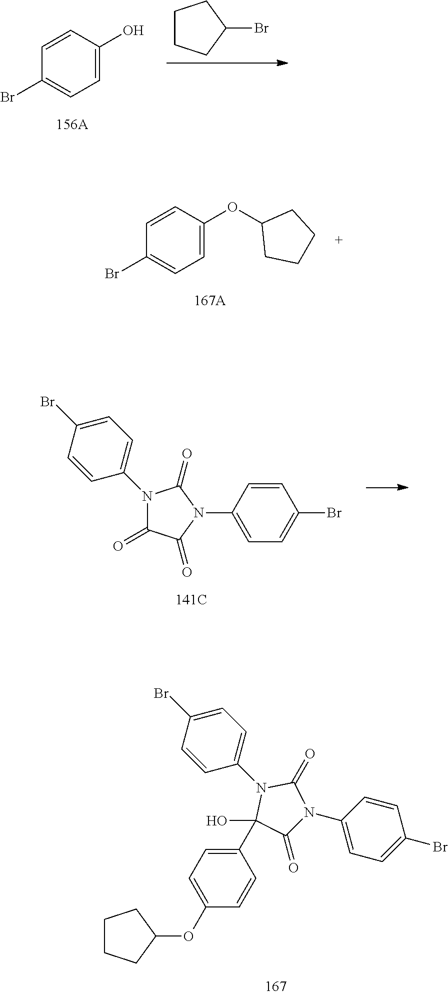 [SMILES: Oc1ccc(Br)cc1 ; BrC1CCCC1 ; Brc1ccc(OC2CCCC2)cc1 ; O=C1C(=O)N(c2ccc(Br)cc2)C(=O)N1c1ccc(Br)cc1 ; O=C1N(c2ccc(Br)cc2)C(=O)C(O)(c2ccc(OC3CCCC3)cc2)N1c1ccc(Br)cc1]

Compounds 167A and 167 were synthesized by employing the procedures described for Compounds 156B and 127 using bromocyclopentane, 167A, and 141C in lieu of bromocyclopropane, 2C, and 1-bromo-4-(tert-butyl)benzene. Compound 167A. LC-MS (ESI) m/z: No signal. ¹H-NMR: (CDCl₃, 400 MHz): δ (ppm) 1.51-1.55 (m, 2H), 1.68-1.82 (m, 6H), 4.59-4.64 (m, 1H), 6.66 (d, J=8.8 Hz, 2H), 7.25 (d, J=9.2 Hz, 2H). Compound 167. LC-MS (ESI) m/z: 1195 [2M+Na]⁺; ¹H-NMR (CDCl₃, 400 MHz): δ (ppm) 1.59-1.66 (m, 2H), 1.76-1.94 (m, 6H), 4.70-4.74 (m, 1H), 6.83 (d, J=8.8 Hz, 2H), 7.28-7.34 (m, 6H), 7.37-7.39 (m, 2H), 7.56 (d, J=8.4 Hz, 2H).

### Example 168

### Synthesis of 1,3-bis(4-(cyclopentyloxy)phenyl)-5-hydroxy-5-phenylimidazolidine-2,4-dione

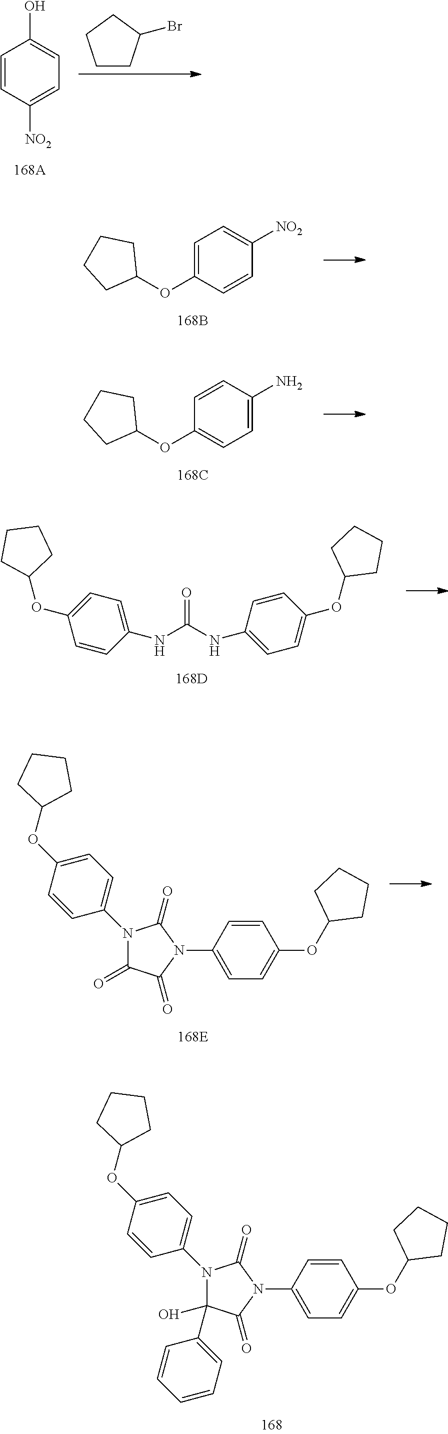

To a mixture of Compound 168A (2 g, 14 mmol) and bromocyclopentane (3.1 g, 21 mmol) in DMF (50 mL) was added potassium carbonate (3.97 g, 28 mmol). The mixture was stirred at 80° C. for 20 hours. The mixture was cooled down to room temperature and diluted with ethyl acetate (300 mL). The organic layer was washed with water (100 mL×4) and brine (100 mL), dried over anhydrous sodium sulfate, filtered, and concentrated to give a crude Compound 168B. LC-MS (ESI) m/z: No. ¹H-NMR (CDCl₃, 400 MHz): δ (ppm) 1.64-1.68 (m, 2H), 1.77-1.97 (m, 6H), 4.84-4.87 (m, 1H), 6.90-6.92 (d, J=9.2 Hz, 2H), 8.16-8.19 (m, 2H).

To a solution of Compound 168B (2.8 g, 13.5 mmol) in ethanol (30 mL) was added Pd(OH)₂/C (200 mg). The mixture was stirred under hydrogen at room temperature for 16 hours. After removal of Pd(OH)₂/C by filtration, the filtrate was evaporated to afford Compound 168C. LC-MS (ESI) m/z: 178 [M+H]⁺.

Compounds 168D, 168E, and 168 were synthesized by employing the procedures described for Compounds 141B, 141C, and 141 using 168C, 168D, and 168E in lieu of 140A, 141B, and 141C. Compound 168D. LC-MS (ESI) m/z: 381 [M+H]⁺. Compound 168E. LC-MS (ESI) m/z: 891 [2M+Na]⁺; ¹H-NMR (CDCl₃, 400 MHz): δ (ppm), 1.60-1.68 (m, 4H), 1.76-1.96 (m, 12H), 4.76-4.80 (m, 2H), 6.96-7.00 (m, 4H), 7.32-7.36 (m, 4H). Compound 168. LC-MS (ESI) m/z: 513 [M+H]⁺; ¹H-NMR (CDCl₃, 400 MHz): δ (ppm) 1.58-1.63 (m, 4H), 1.76-1.93 (m, 12H), 4.63-4.66 (m, 2H), 4.74-4.78 (m, 1H), 6.69 (d, J=9.2 Hz, 2H), 6.89 (d, J=8.8 Hz, 2H), 7.14 (d, J=8.8 Hz, 2H), 7.24 (d, J=8.4 Hz, 2H), 7.34-7.35 (m, 3H), 7.45-7.48 (m, 2H).

### Example 169

### Synthesis of 1,3-bis(4-bromophenyl)-5-(4-(cyclohexyloxy)phenyl)-5-hydroxyimidazolidine-2,4-dione

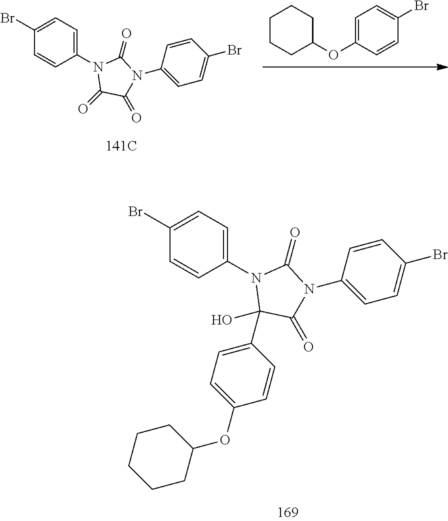

Compound 169 was synthesized by employing the procedure described for Compound 127 using 1-bromo-4-(cyclohexyloxy)benzene and 141C in lieu of 1-bromo-4-(tert-butyl)benzene and 2C. LC-MS (ESI) m/z: 1223 [2M+Na]⁺; ¹H-NMR (DMSO-d₆, 400 MHz): δ (ppm) 1.24 (m, 1H), 1.33-1.39 (m, 4H), 1.51-1.53 (m, 1H), 1.66-1.69 (m, 2H), 1.88 (s, 2H), 4.32 (m, 1H), 6.89 (d, J=8.8 Hz, 2H), 7.45-7.53 (m, 8H), 7.76 (d, J=8.8 Hz, 2H), 8.26 (s, 1H).

### Example 170

### Synthesis of 1,3-bis(4-chlorophenyl)-4-hydroxy-5-methyl-5-propyl-4-(3-(trifluoromethoxy)phenyl)imidazolidin-2-one, (5R)-1,3-bis(4-chlorophenyl)-4-hydroxy-5-methyl-5-propyl-4-(3-(trifluoromethoxy)phenyl)imidazolidin-2-one, and (5S)-1,3-bis(4-chlorophenyl)-4-hydroxy-5-methyl-5-propyl-4-(3-(trifluoromethoxy)phenyl)imidazolidin-2-one

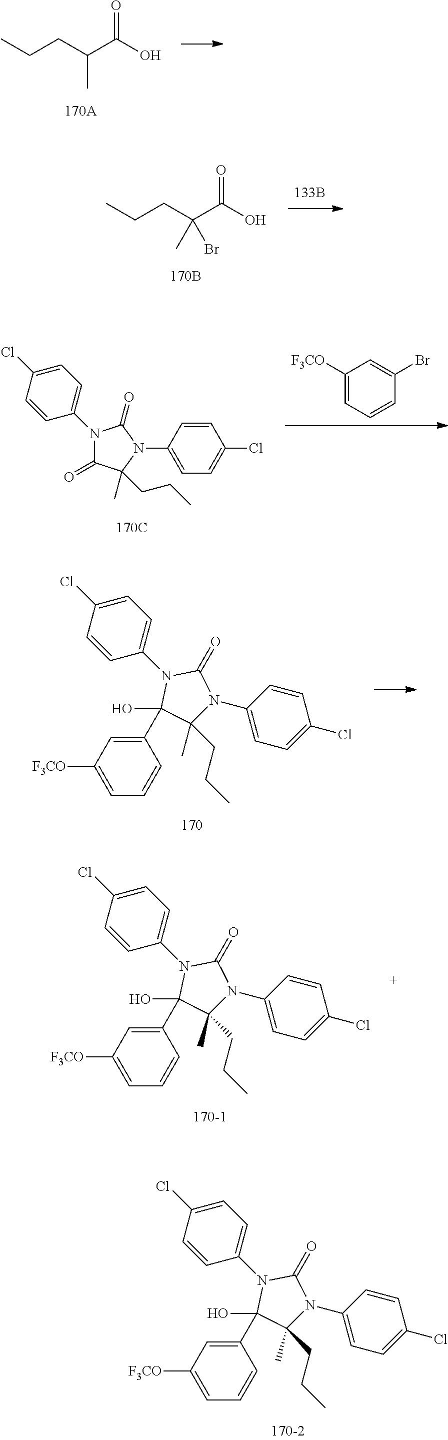

Compounds 170B, 170C, and 170 were synthesized by employing the procedures described for Compounds 163B, 120D, and 125 using Compounds 170A, 133B, 170B, 170C, and 1-bromo-3-(trifluoromethoxy)benzene in lieu of Compounds 163A, 2-bromo-2-methylpropanoic acid, 120C, 2C, and 3-bromo-N,N-dimethylaniline. Compound 170B. ¹H-NMR (400 MHz, CDCl₃): δ (ppm) 0.98 (t, J=7.6 Hz, 3H), 1.37-1.53 (m, 2H), 1.91 (s, 3H), 2.09-2.13 (m, 2H). Compound 170C. LC-MS (ESI) m/z: 377 [M+H]⁺. Compound 170. LC-MS (ESI) m/z: 539 [M+H]⁺. ¹H-NMR (400 MHz, CDCl₃): δ (ppm) 0.87 (t, J=6.8 Hz, 3H), 0.92 (s, 3H), 1.03-1.07 (m, 1H), 1.38-1.44 (m, 1H), 1.64-1.72 (m, 1H), 1.85-1.93 (m, 1H), 3.43 (s, 1H), 7.15-7.20 (m, 5H), 7.27-7.30 (m, 2H), 7.33-7.40 (m, 5H).

Compound 170 was separated by using chiral HPLC to give Compound 170-1 and Compound 170-2. Compound 170-1: LC-MS (ESI) m/z: 539 [M+H]⁺; ¹H-NMR (400 MHz, CDCl₃): δ (ppm) 0.86 (t, J=6.8 Hz, 3H), 0.91 (s, 3H), 1.01-1.05 (m, 1H), 1.35-1.44 (m, 1H), 1.62-1.70 (m, 1H), 1.84-1.92 (m, 1H), 3.60 (s, 1H), 7.13-7.19 (m, 5H), 7.28-7.29 (m, 2H), 7.31-7.39 (m, 5H). Chiral separation conditions: MeOH contained 0.2% Methanol Ammonia; Cellulose-SC (4.6*100 mm, 5 μm); retention time: 1.04 minutes (100%). Compound 170-2: LC-MS (ESI) m/z: 539 [M+H]⁺; ¹H-NMR (400 MHz, CDCl₃): δ (ppm) 0.86 (t, J=6.8 Hz, 3H), 0.91 (s, 3H), 0.99-1.08 (m, 1H), 1.37-1.44 (m, 1H), 1.63-1.70 (m, 1H), 1.84-1.92 (m, 1H), 3.58 (s, 1H), 7.14-7.19 (m, 5H), 7.28-7.30 (m, 2H), 7.34-7.39 (m, 5H). Chiral separation conditions: MeOH contained 0.2% Methanol Ammonia; Cellulose-SC (4.6*100 mm, 5 μm); retention time: 2.11 minutes (99%).

### Example 171

### Synthesis of 1,3-bis(4-bromophenyl)-5-(3-chloro-4-cyclopropoxyphenyl)-5-hydroxyimidazolidine-2,4-dione

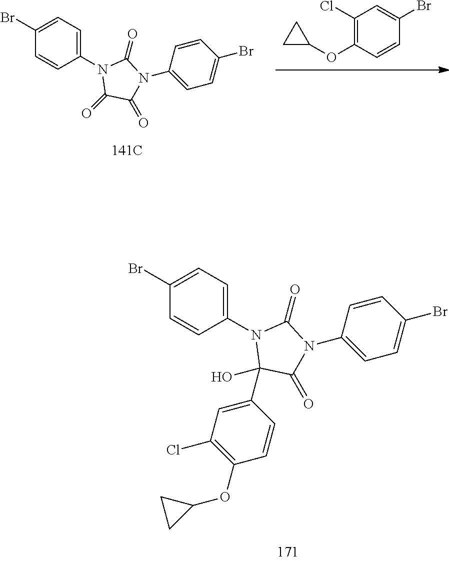

Compound 171 was synthesized by employing the procedure described for Compound 127 using 4-bromo-2-chloro-1-cyclopropoxybenzene and 141C in lieu of 1-bromo-4-(tert-butyl)benzene and 2C. LC-MS (ESI) m/z: 591 [M+H]⁺; ¹H-NMR (CD₃OD, 400 MHz): δ (ppm) 0.66-0.75 (m, 4H), 3.75-3.80 (m, 1H), 7.29-7.31 (m, 1H), 7.35-7.39 (m, 5H), 7.45-7.47 (m, 2H), 7.53-7.54 (m, 1H), 7.61-7.63 (m, 2H).

### Example 172

### Synthesis of 1,3-bis(4-bromophenyl)-5-(4-fluorophenyl)-5-hydroxyimidazolidine-2,4-dione

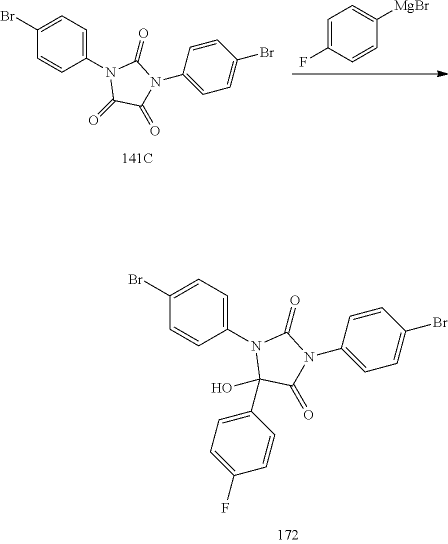

Compound 172 was synthesized by employing the procedure described for Compound 141 using (4-fluorophenyl)magnesium bromide in lieu of phenylmagnesium bromide. LC-MS (ESI) m/z: No. ¹H-NMR (CD₃OD, 400 MHz): δ (ppm) 7.12 (t, J=8.7 Hz, 2H), 7.42-7.52 (m, 6H), 7.59-7.74 (m, 4H).

### Example 173

### Synthesis of 1,3-bis(4-bromophenyl)-5-hydroxy-5-(4-(piperidin-1-yl)phenyl)imidazolidine-2,4-dione

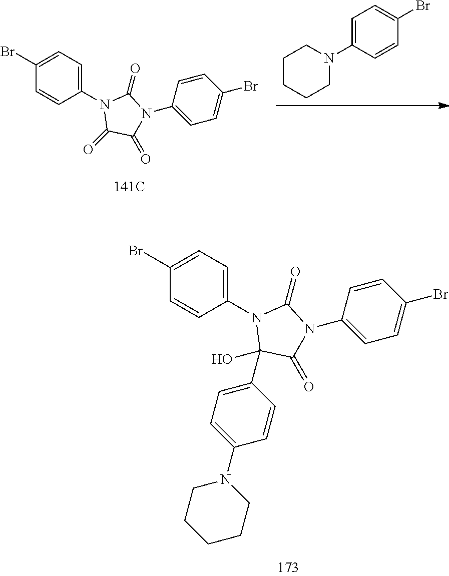

Compound 173 was synthesized by employing the procedure described for Compound 127 using 1-(4-bromophenyl)piperidine and 141C in lieu of 1-bromo-4-(tert-butyl)benzene and 2C. LC-MS (ESI) m/z: 585 [M+H]⁺; ¹H-NMR (CDCl₃, 400 MHz): δ (ppm) 1.65 (s, 2H), 1.94 (s, 4H), 3.29 (s, 4H), 7.24 (d, J=8.8 Hz, 2H), 7.29 (d, J=8.8 Hz, 2H), 7.35 (d, J=8.8 Hz, 2H), 7.51 (d, J=8.8 Hz, 4H), 7.59 (d, J=8.8 Hz, 2H).

### Example 174

### Synthesis of 1,3-bis(4-bromophenyl)-5-(4-cyclobutoxyphenyl)-5-hydroxyimidazolidine-2,4-dione

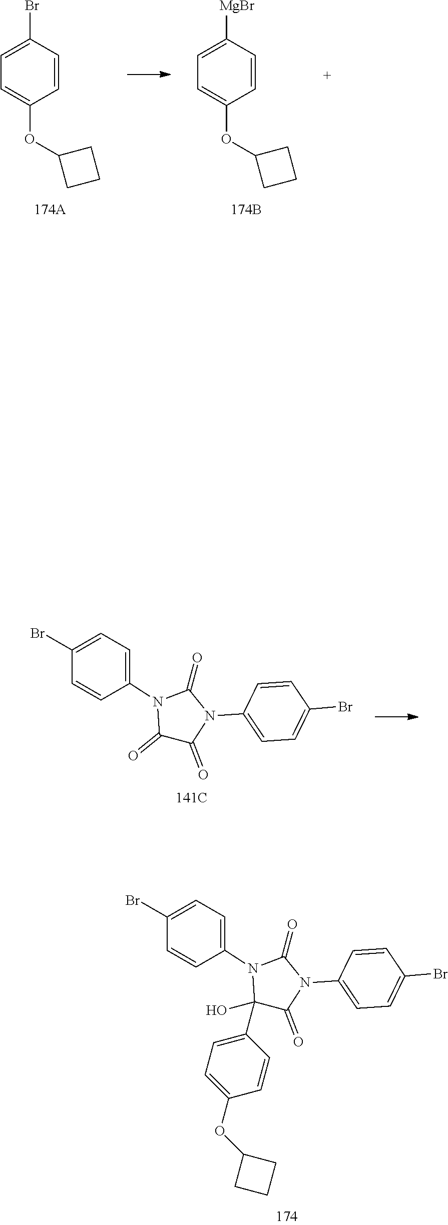

To a mixture of magnesium (159 mg, 6.6 mmol) and a small amount of iodine in anhydrous THF (10 mL) were added 1,2-dibromomethane (0.1 mL) and heated at 50° C. for 10 minutes. To the mixture was dropped a solution of Compound 174A (1 g, 4.4 mmol) in THF (2 mL) and stirred at 80° C. for 2 hours. The resulting Grignard reagent 174B was cooled down to room temperature and used directly in the next step.

Compound 174 was synthesized by employing the procedure described for Compound 141 using Grignard reagent 174B in lieu of phenylmagnesium bromide. LC-MS (ESI) m/z: 1163 [2M+Na]⁺; ¹H-NMR (CDCl₃, 400 MHz): δ (ppm) 1.64-1.71 (m, 1H), 1.82-1.90 (m, 1H), 2.10-2.19 (m, 2H), 2.39-2.46 (m, 2H), 4.58-4.64 (m, 1H), 6.77 (d, J=8.4 Hz, 2H), 7.27-7.29 (m, 2H), 7.30-7.34 (m, 4H), 7.37-7.38 (m, 2H), 7.56 (d, J=8.0 Hz, 2H).

### Example 175

### Synthesis of 1,3-bis(4-bromophenyl)-5-(2,3-dihydrobenzo[b][1,4]dioxin-6-yl)-5-hydroxyimidazolidine-2,4-dione

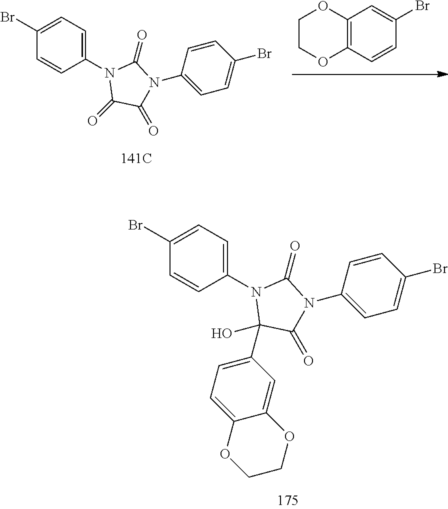

Compound 175 was synthesized by employing the procedure described for Compound 127 using 6-bromo-2,3-dihydrobenzo[b][1,4]dioxine and 141C in lieu of 1-bromo-4-(tert-butyl)benzene and 2C. LC-MS (ESI) m/z: 541 [M-OH]⁺; ¹H-NMR (CD₃OD, 400 MHz): δ (ppm) 4.22 (s, 4H), 6.81-6.83 (m, 1H), 6.98-7.00 (m, 1H), 7.07-7.08 (m, 1H), 7.43-7.45 (m, 4H), 7.51-7.53 (m, 2H), 7.68-7.70 (m, 2H).

### Example 176

### Synthesis of 4-hydroxy-5,5-dimethyl-1,3-di-p-tolyl-4-(3-(trifluoromethoxy)phenyl)-imidazolidin-2-one

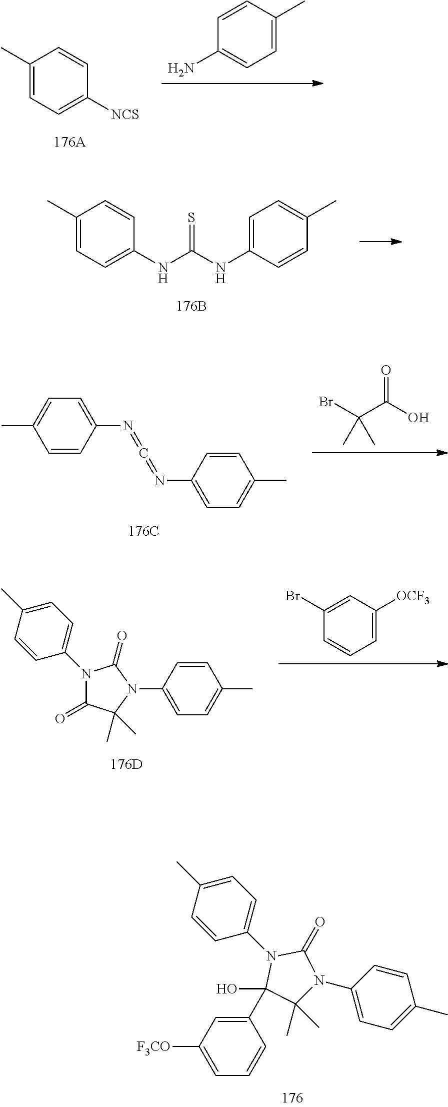

Compounds 176B, 176C, 176D, and 176 were synthesized by employing the procedures described for Compounds 120B, 120C, 120D, and 125 using p-toluidine, Compounds 176A, 176B, 176C, and 1-bromo-3-(trifluoromethoxy)benzene in lieu of Compounds 120A, 1-bromo-4-isocyanatobenzene, 120B, 120C, 2C, and 3-bromo-N,N-dimethylaniline. Compound 176B. LC-MS (ESI) m/z: 257 [M+H]⁺; ¹H-NMR (CDCl₃, 400 MHz): δ (ppm) 2.36 (s, 6H), 7.19-7.26 (m, 8H), 7.70 (s, 2H). Compound 176C. ¹H-NMR (CDCl₃, 400 MHz): δ (ppm) 2.33 (s, 6H), 7.06 (d, J=8.0 Hz, 4H), 7.12 (d, J=8.0 Hz, 4H). Compound 176D. LC-MS (ESI) m/z: 309 [M+H]⁺; ¹H-NMR (CDCl₃, 400 MHz): δ (ppm) 1.52 (s, 6H), 2.38 (s, 3H), 2.40 (s, 3H), 7.19 (d, J=8.0 Hz, 2H), 7.25-7.28 (m, 4H), 7.36 (d, J=8.0 Hz, 2H). Compound 176. LC-MS (ESI) m/z: 471 [M+H]⁺; ¹H-NMR (CDCl₃, 400 MHz): δ (ppm) 0.85 (s, 3H), 1.36 (s, 3H), 2.25 (s, 3H), 2.38 (s, 3H), 3.21 (s, 1H), 7.03 (d, J=8.0 Hz, 2H), 7.14-7.24 (m, 5H), 7.31-7.42 (m, 5H).

### Example 177

### Synthesis of 3-(1,3-bis(4-chlorophenyl)-4-hydroxy-5-methyl-2-oxo-5-(trifluoromethyl)imidazolidin-4-yl)benzonitrile, 3-((5S)-1,3-bis(4-chlorophenyl)-4-hydroxy-5-methyl-2-oxo-5-(trifluoromethyl)imidazolidin-4-yl)benzonitrile, and 3-((5R)-1,3-bis(4-chlorophenyl)-4-hydroxy-5-methyl-2-oxo-5-(trifluoromethyl)imidazolidin-4-yl)benzonitrile

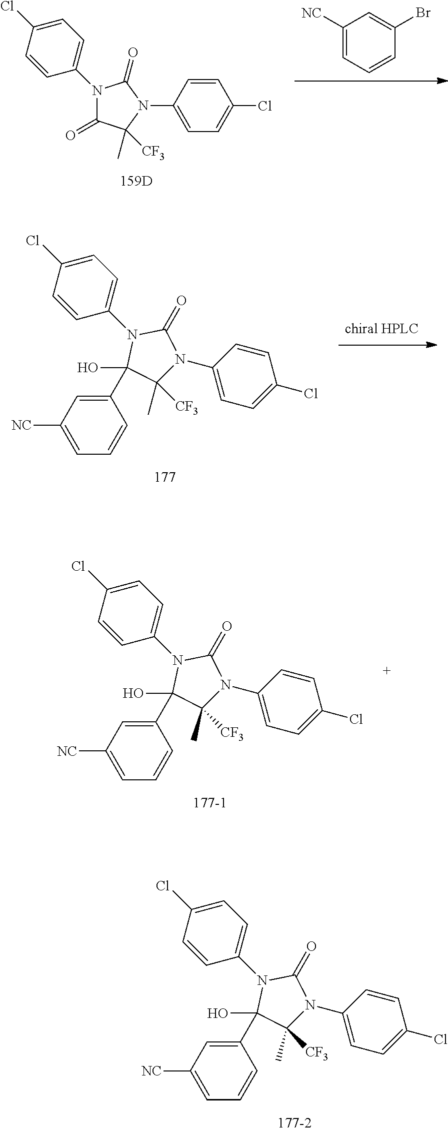 [SMILES: CC1(C(F)(F)F)C(=O)N(c2ccc(Cl)cc2)C(=O)N1c1ccc(Cl)cc1 ; N#Cc1cccc(Br)c1 ; CC1(C(F)(F)F)N(c2ccc(Cl)cc2)C(=O)N(c2ccc(Cl)cc2)C1(O)c1cccc(C#N)c1 ; C[C@]1(C(F)(F)F)N(c2ccc(Cl)cc2)C(=O)N(c2ccc(Cl)cc2)C1(O)c1cccc(C#N)c1 ; C[C@@]1(C(F)(F)F)N(c2ccc(Cl)cc2)C(=O)N(c2ccc(Cl)cc2)C1(O)c1cccc(C#N)c1]

Compound 177 was synthesized by employing the procedure described for Compound 125 using Compound 159D and 3-bromobenzonitrile in lieu of Compounds 2C and 3-bromo-N,N-dimethylaniline. LC-MS (ESI) m/z: 506 [M+H]⁺; ¹H-NMR (CDCl₃, 400 MHz): δ (ppm) 1.05 (s, 3H), 3.75 (s, 1H), 7.19 (d, J=8.8 Hz, 4H), 7.26-7.29 (m, 2H), 7.30 (d, J=8.8 Hz, 2H), 7.42 (d, J=8.8 Hz, 2H), 7.50-7.55 (m, 1H), 7.70 (d, J=7.2 Hz, 1H).

Compound 177 was separated by using chiral HPLC to give Compound 177-1 and Compound 177-2. Compound 177-1: LC-MS (ESI) m/z: 506 [M+H]⁺; ¹H-NMR (CDCl₃, 400 MHz): δ (ppm) 1.05 (s, 3H), 3.93 (s, 1H), 7.18-7.22 (m, 4H), 7.26-7.31 (m, 3H), 7.41-7.43 (d, J=8.8 Hz, 2H), 7.43-7.50 (m, 1H), 7.70 (d, J=7.2 Hz, 2H). Chiral separation conditions: MeOH contained 0.2% Methanol Ammonia; (R, R)-Whelk-Ol (4.6*250 mm, 5 μm); retention time: 2.22 minutes. Compound 177-2: LC-MS (ESI) m/z: 506 [M+H]⁺; ¹H-NMR (CDCl₃, 400 MHz): δ (ppm) 1.05 (s, 3H), 4.04 (s, 1H), 7.18-7.22 (m, 4H), 7.26-7.31 (m, 3H), 7.40-7.43 (d, J=8.8 Hz, 2H), 7.43-7.52 (m, 1H), 7.67 (d, J=7.2 Hz, 2H), Chiral separation conditions: MeOH contained 0.2% Methanol Ammonia; (R, R)-Whelk-Ol (4.6*250 mm, 5 μm); retention time: 3.66 minutes.

### Example 178

### Synthesis of 1,3-bis(4-bromophenyl)-5-(4′-fluoro-[1,1′-biphenyl]-4-yl)-5-hydroxyimidazolidine-2,4-dione

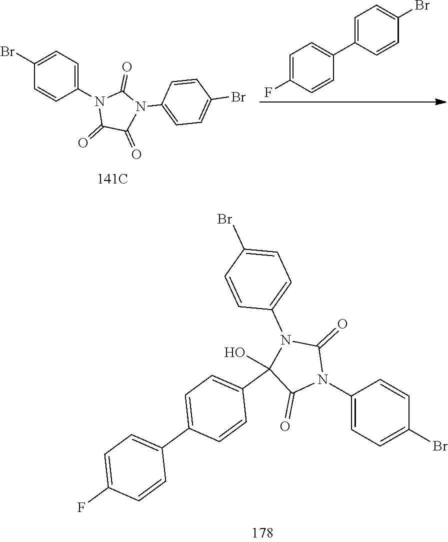

Compound 178 was synthesized by employing the procedure described for Compound 127 using 4-bromo-4′-fluoro-1,1′-biphenyl and 141C in lieu of 1-bromo-4-(tert-butyl)benzene and 2C. LC-MS (ESI) m/z: 1211 [2M+Na]⁺; ¹H-NMR (CDCl₃, 400 MHz): δ (ppm) 7.13 (t, J=8.4 Hz, 2H), 7.38-7.42 (m, 6H), 7.49-7.57 (m, 6H), 7.62 (d, J=10.0 Hz, 2H).

### Example 179

### Synthesis of 1,3-bis(4-bromophenyl)-5-hydroxy-5-(3-methoxyphenyl)imidazolidine-2,4-dione

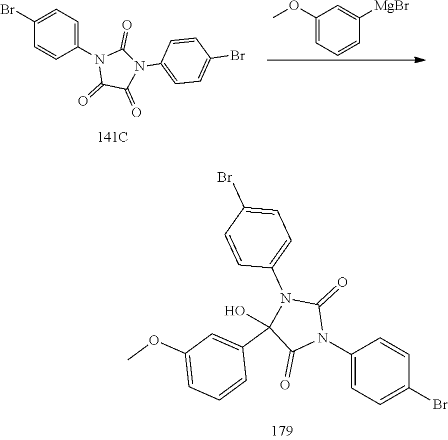

Compound 179 was synthesized by employing the procedure described for Compound 141 using (3-methoxyphenyl)magnesium bromide in lieu of phenylmagnesium bromide. LC-MS (ESI) m/z: 531 [M+H]⁺; ¹H-NMR (CDCl₃, 400 MHz): δ (ppm) 3.76 (s, 3H), 6.89 (dd, J=8.0 Hz, 2.0 Hz, 1H), 6.95-6.97 (m, 1H), 7.05 (t, J=2.0 Hz, 1H), 7.12-7.14 (m, 2H), 7.22-7.27 (m, 3H), 7.31-7.34 (m, 2H), 7.48-7.50 (m, 2H).

### Example 180

### Synthesis of 4,4′-(4-hydroxy-5-methyl-2-oxo-4-(3-(trifluoromethoxy)phenyl)imidazolidine-1,3-diyl)dibenzonitrile, 4,4′-((5S)-4-hydroxy-5-methyl-2-oxo-4-(3-(trifluoromethoxy)phenyl)imidazolidine-1,3-diyl)dibenzonitrile, and 4,4′-((5R)-4-hydroxy-5-methyl-2-oxo-4-(3-(trifluoromethoxy)phenyl)imidazolidine-1,3-diyl)dibenzonitrile

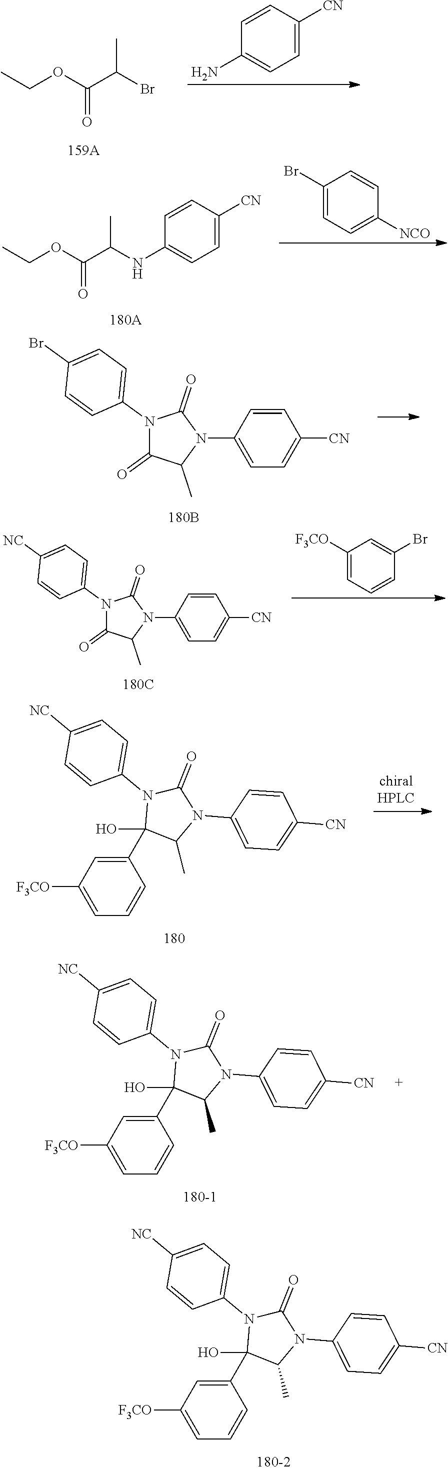

Compounds 180A and 180B were synthesized by employing the procedures described for Compounds 2B and 2C using Compounds 159A, 4-aminobenzonitrile, and 180A in lieu of Compounds 2A, 4-bromoaniline, and 2B. Compound 180A. LC-MS (ESI) m/z: 219 [M+H]⁺; ¹H-NMR (CDCl₃, 400 MHz): δ (ppm) 1.29 (t, J=7.2 Hz, 3H), 1.50 (d, J=7.2 Hz, 3H), 4.13-4.26 (m, 3H), 4.73 (d, J=7.6 Hz, 1H), 6.57 (d, J=8.8 Hz, 2H), 7.44 (d, J=8.8 Hz, 2H). Compound 180B. LC-MS (ESI) m/z: 370 [M+H]⁺; ¹H-NMR (CDCl₃, 400 MHz): δ (ppm) 1.46 (d, J=6.8 Hz, 3H), 5.15 (q, J=6.8 Hz, 1H), 7.43 (d, J=8.8 Hz, 2H), 7.72-7.75 (m, 2H), 7.83 (d, J=8.4 Hz, 2H), 7.92 (d, J=9.2 Hz, 2H).

A mixture of Compound 180B (500 mg, 1.4 mmol), Zn(CN)₂ (164 mg, 1.4 mmol), Pd₂(dba)₃ (130 mg, 0.14 mmol), and DPPF (194 mg, 0.35 mmol) in DMF (15 mL) was stirred at 150° C. under nitrogen for 4 hours. The reaction mixture was cooled down to room temperature and filtered through Celite. The filtrate was diluted with water (100 mL) and extracted with ethyl acetate (100 mL×2). The combined extracts were washed with water (100 mL×2) and brine (100 mL), dried over anhydrous sodium sulfate, filtered, and concentrated. The residue was purified with flash column chromatography on silica gel (ethyl acetate in petroleum ether, from 50% to 100% v/v) to give a crude product, which was triturated in ethyl acetate (20 mL) to yield Compound 180C: LC-MS (ESI) m/z: 317 [M+H]⁺; ¹H-NMR (DMSO-d₆, 400 MHz): δ (ppm) 1.46 (d, J=6.4 Hz, 3H), 5.18 (q, J=6.8 Hz, 1H), 7.70 (d, J=8.4 Hz, 2H), 7.84 (d, J=8.8 Hz, 2H), 7.94 (d, J=8.8 Hz, 2H), 8.03 (d, J=8.4 Hz, 2H).

Compound 180 was synthesized by employing the procedure described for Compound 125 using Compound 180C and 1-bromo-3-(trifluoromethoxy)benzene in lieu of Compounds 2C and 3-bromo-N,N-dimethylaniline. LC-MS (ESI) m/z: 479 [M+H]⁺; ¹H-NMR (CDCl₃, 400 MHz): δ (ppm) 0.85, 1.34 (d, J=6.4 Hz, 3H), 3.71, 4.53 (s, 1H), 4.32, 4.47 (q, J=6.8 Hz, 1H), 7.20-7.21 (m, 1H), 7.35-7.80 (m, 11H).

Compound 180 was separated by using chiral HPLC to give Compound 180-1 and Compound 180-2. Compound 180-1: LC-MS (ESI) m/z: 479 [M+H]⁺; ¹H-NMR (CDCl₃, 400 MHz): δ (ppm) 0.84, 1.34 (d, J=6.4 Hz, 3H), 4.11, 4.99 (s, 1H), 4.31, 4.46 (q, J=6.8 Hz, 1H), 7.19-7.20 (m, 1H), 7.25-7.71 (m, 11H). Chiral separation conditions: MeOH contained 0.2% methanol ammonia; Enantiopak OD (4.6*100 mm, 5 μm); retention time: 1.09 minutes (77%), 1.98 minutes (23%). Compound 180-2: LC-MS (ESI) m/z: 479 [M+H]⁺; ¹H-NMR (CDCl₃, 400 MHz): δ (ppm) 0.86, 1.35 (d, J=6.4 Hz, 3H), 3.74, 4.56 (s, 1H), 4.32, 4.46 (q, J=6.8 Hz, 1H), 7.20-7.21 (m, 1H), 7.35-7.74 (m, 11H). Chiral separation conditions: co-solvent: MeOH contained 0.2% methanol ammonia; Enantiopak OD (4.6*100 mm, 5 μm); retention time: 1.57 minutes (24%), 2.09 minutes (76%).

### Example 181

### Synthesis of 1,3-bis(3-bromophenyl)-5-hydroxy-5-phenylimidazolidine-2,4-dione

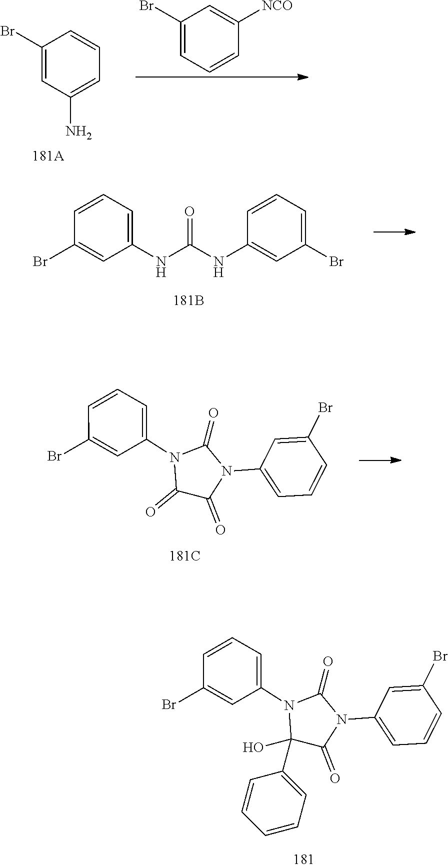

Compounds 181B, 181C, and 181 were synthesized by employing the procedures described for Compounds 1, 141C, and 141 using 181A, 1-bromo-3-isocyanatobenzene in the presence of triethylamine, 181B, and 181C in lieu of 140A, 1-bromo-4-isocyanatobenzene, 141B, and 141C. Compound 181B. LC-MS (ESI) m/z: non-ionizable compound under routine conditions used. Compound 181C. LC-MS (ESI) m/z: non-ionizable compound under routine conditions used; ¹H-NMR (DMSO-d₆, 400 MHz): δ (ppm) 7.49-7.51 (m, 2H), 7.58 (t, J=8.0 Hz, 2H), 7.66 (t, J=1.8 Hz, 2H), 7.73-7.76 (m, 2H). Compound 181. LC-MS (ESI) m/z: non-ionizable compound under routine conditions used; ¹H-NMR (CD₃OD, 400 MHz): δ (ppm) 7.18 (t, J=8.1 Hz, 1H), 7.29-7.68 (m, 10H), 7.75 (t, J=1.8 Hz, 1H), 7.83 (t, J=1.9 Hz, 1H).

### Example 182

### Synthesis of 1,3-bis(4-bromophenyl)-5-(4-butoxyphenyl)-5-hydroxyimidazolidine-2,4-dione

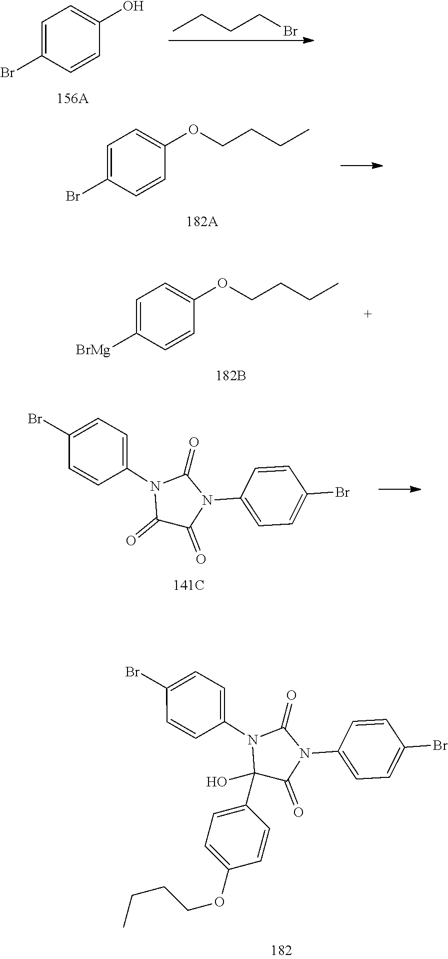

Compounds 182A, 182B, and 182 were synthesized by employing the procedures described for Compounds 156B, 174B, and 141 using 1-bromobutane, heating at 80° C., Compounds 182A, and 182B in lieu of bromocyclopropane, heating at 150° C., Compound 174A, and phenylmagnesium bromide. Compound 182A. LC-MS (ESI) m/z: non-ionizable compound under routine conditions used; ¹H-NMR (CDCl₃, 400 MHz): δ (ppm) 0.96 (t, J=7.2 Hz, 3H), 1.44-1.50 (m, 2H), 1.71-1.78 (m, 2H), 3.91 (t, J=6.4 Hz, 2H), 6.77 (d, J=8.8 Hz, 2H), 7.35 (d, J=8.8 Hz, 2H). Compound 182B as a crude Grignard reagent was used directly in the next step. Compound 182. LC-MS (ESI) m/z: 573 [M+H]⁺; ¹H-NMR (CDCl₃, 400 MHz): δ (ppm) 0.96 (t, J=5.7 Hz, 3H), 1.42-1.51 (m, 2H), 1.71-1.78 (m, 2H), 3.93 (t, J=6.4 Hz, 2H), 4.39 (s, 1H), 6.86 (d, J=9.2 Hz, 2H), 7.29-7.39 (m, 8H), 7.57 (d, J=6.8 Hz, 2H).

### Example 183

### Synthesis of 1,3-bis(4-bromophenyl)-5-(4-(cyclopentylmethoxy)phenyl)-5-hydroxyimidazolidine-2,4-dione

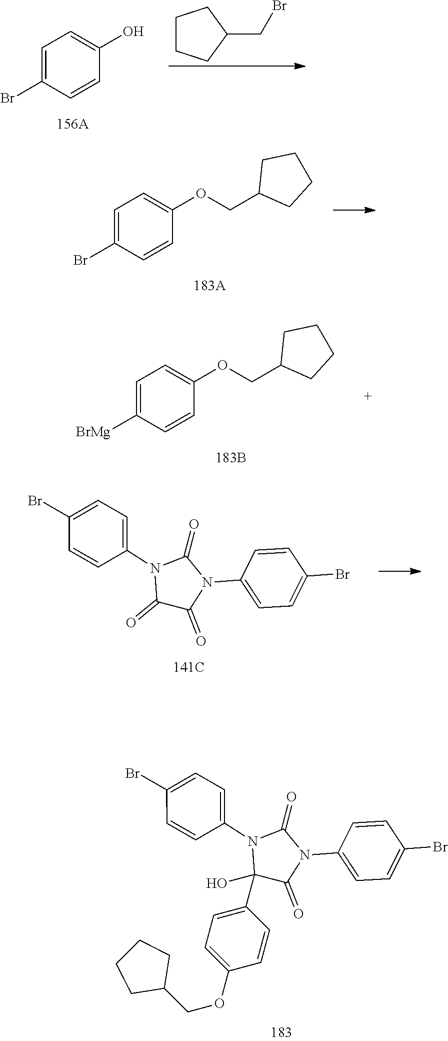

Compounds 183A, 183B, and 183 were synthesized by employing the procedures described for Compounds 156B, 174B, and 141 using (bromomethyl)cyclopentane, heating at 80° C., Compounds 183A, 183B, and at −20° C. in lieu of bromocyclopropane, heating at 150° C., Compound 174A, phenylmagnesium bromide, and −78° C. Compound 183A. LC-MS (ESI) m/z: non-ionizable compound under routine conditions used; ¹H-NMR (CDCl₃, 400 MHz): δ (ppm) 1.31-1.35 (m, 2H), 1.57-1.64 (m, 4H), 1.80-1.85 (m, 2H), 2.30-2.34 (m, 1H), 3.78 (d, J=6.8 Hz, 2H), 6.77 (d, J=8.8 Hz, 2H), 7.35 (d, J=8.8 Hz, 2H).

Compound 183B as a crude Grignard reagent was cooled to room temperature and used directly in the next step. Compound 183. LC-MS (ESI) m/z: 599 [M+H]⁺; ¹H-NMR (CDCl₃, 400 MHz): δ (ppm) 1.30-1.37 (m, 2H), 1.54-1.65 (m, 4H), 1.79-1.86 (m, 2H), 2.27-2.37 (m, 1H), 3.78 (d, J=7.2 Hz, 2H), 4.29 (s, 1H), 6.86 (d, J=8.8 Hz, 2H), 7.30-7.39 (m, 8H), 7.58 (d, J=8.8 Hz, 2H).

### Example 184

### Synthesis of 1,3-bis(4-bromophenyl)-5-(4-(cyclopropylmethoxy)phenyl)-5-hydroxyimidazolidine-2,4-dione

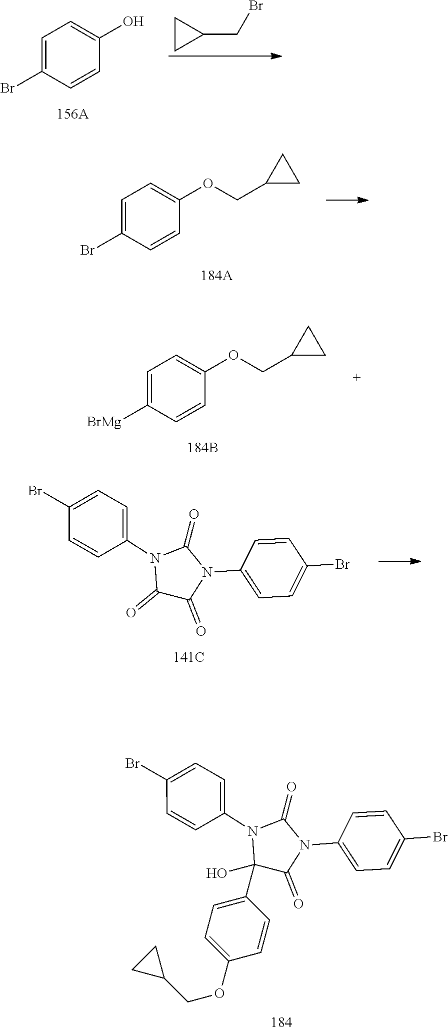

Compounds 184A, 184B, and 184 were synthesized by employing the procedures described for Compounds 156B, 174B, and 141 using (bromomethyl)cyclopentane, heating at 80° C., Compounds 184A, 184B, and at −20° C. in lieu of bromocyclopropane, heating at 150° C., Compound 174A, phenylmagnesium bromide, and −78° C. Compound 184A. LC-MS (ESI) m/z: non-ionizable compound under routine conditions used; ¹H-NMR (CDCl₃, 400 MHz): δ (ppm) 0.32-0.35 (m, 2H), 0.63-0.66 (m, 2H), 1.21-1.30 (m, 1H), 3.75 (d, J=7.2 Hz, 2H), 6.77 (d, J=8.8 Hz, 2H), 7.35 (d, J=8.8 Hz, 2H).

Compound 184B as a crude Grignard reagent was cooled to room temperature and used directly in the next step. Compound 184. LC-MS (ESI) m/z: 571 [M+1]⁺; ¹H-NMR (CDCl₃, 400 MHz): δ (ppm) 0.31-0.35 (m, 2H), 0.62-0.66 (m, 2H), 3.77 (d, J=6.8 Hz, 2H), 4.30 (s, 1H), 6.87 (d, J=8.8 Hz, 2H), 7.30-7.39 (m, 8H), 7.58 (d, J=8.8 Hz, 2H).

### Example 185

### Synthesis of 3-(1,3-bis(4-chlorophenyl)-5-ethyl-4-hydroxy-5-methyl-2-oxoimidazolidin-4-yl)benzonitrile, 3-((5R)-1,3-bis(4-chlorophenyl)-5-ethyl-4-hydroxy-5-methyl-2-oxoimidazolidin-4-yl)benzonitrile, and 3-((5S)-1,3-bis(4-chlorophenyl)-5-ethyl-4-hydroxy-5-methyl-2-oxoimidazolidin-4-yl)benzonitrile

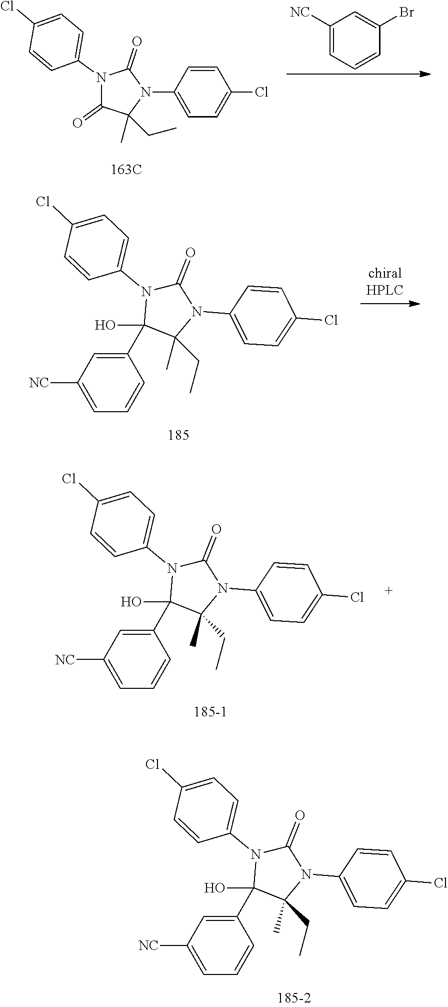 [SMILES: CCC1(C)C(=O)N(c2ccc(Cl)cc2)C(=O)N1c1ccc(Cl)cc1 ; N#Cc1cccc(Br)c1 ; CCC1(C)N(c2ccc(Cl)cc2)C(=O)N(c2ccc(Cl)cc2)C1(O)c1cccc(C#N)c1 ; CC[C@@]1(C)N(c2ccc(Cl)cc2)C(=O)N(c2ccc(Cl)cc2)C1(O)c1cccc(C#N)c1 ; CC[C@]1(C)N(c2ccc(Cl)cc2)C(=O)N(c2ccc(Cl)cc2)C1(O)c1cccc(C#N)c1]

Compound 185 was synthesized by employing the procedure described for Compound 125 using Compounds 163C and 3-bromobenzonitrile in lieu of Compounds 2C and 3-bromo-N,N-dimethylaniline.

Compound 185 was separated by using chiral HPLC to give Compound 185-1 and Compound 185-2. Compound 185-1: LC-MS (ESI) m/z: 466 [M+H]⁺; ¹H-NMR (400 MHz, CDCl₃): δ (ppm) 0.82 (t, J=7.6 Hz, 3H), 0.93 (s, 3H), 1.77-1.81 (m, 1H), 1.98-2.02 (m, 1H), 3.79 (s, 1H), 7.14-7.20 (m, 4H), 7.27-7.29 (m, 2H), 7.38-7.45 (m, 4H), 7.60-7.62 (m, 1H), 7.86 (br, 1H). Chiral separation conditions: MeOH contained 0.2% Methanol Ammonia; CC4 (4.6*100 mm, 5 μm); retention time: 0.98 minutes (100%). Compound 185-2: LC-MS (ESI) m/z: 466 [M+H]⁺; ¹H-NMR (400 MHz, CDCl₃): δ (ppm) 0.82 (t, J=7.6 Hz, 3H), 0.93 (s, 3H), 1.75-1.80 (m, 1H), 1.97-2.03 (m, 1H), 3.81 (s, 1H), 7.13-7.20 (m, 4H), 7.26-7.28 (m, 2H), 7.38-7.44 (m, 4H), 7.59-7.61 (m, 1H), 7.86 (br, 1H). Chiral separation conditions: MeOH contained 0.2% Methanol Ammonia; CC4 (4.6*100 mm, 5 μm); retention time: 1.86 minutes (100%).

### Example 186

### Synthesis of 5-(1,3-bis(4-chlorophenyl)-4-hydroxy-5,5-dimethyl-2-oxoimidazolidin-4-yl)nicotinonitrile

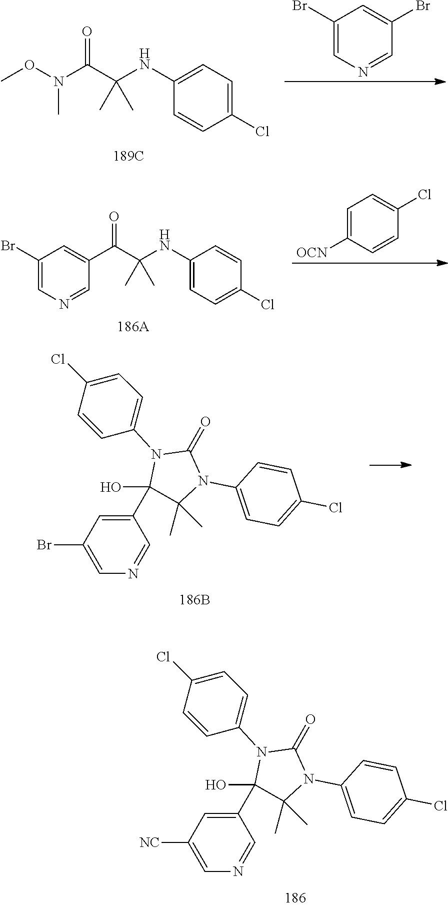

Compounds 186A, 186B, and 186 were synthesized by employing the procedures described for Compounds 59B, 128C, and 180C using Compounds 189C, 3,5-dibromopyridine, 186A, and 186B in lieu of N-methoxy-N-methylacetamide, Compounds 59A, 128B, and 180B. Compound 186A. LC-MS (ESI) m/z: 353 [M+H]⁺; ¹H-NMR (CDCl₃, 400 MHz): δ (ppm) 1.60 (s, 6H), 6.39 (d, J=8.8 Hz, 2H), 7.04 (d, J=8.8 Hz, 2H), 8.58 (s, 1H), 8.75 (d, J=2.0 Hz, 1H), 8.45 (d, J=1.6 Hz, 1H). Compound 186B. LC-MS (ESI) m/z: 506 [M+H]⁺; ¹H-NMR (CDCl₃, 400 MHz): δ (ppm) 0.81 (s, 3H), 1.29 (s, 3H), 4.73 (s, 1H), 7.08 (d, J=8.4 Hz, 2H), 7.18 (d, J=8.8 Hz, 2H), 7.31-7.35 (m, 4H), 7.94 (s, 1H), 8.42 (s, 1H), 8.58 (d, J=2.4 Hz, 1H). Compound 186. LC-MS (ESI) m/z: 453 [M+H]⁺; ¹H-NMR (CD₃OD, 400 MHz): δ (ppm) 0.94 (s, 3H), 1.37 (s, 3H), 7.28 (d, J=9.2 Hz, 2H), 7.42 (d, J=8.8 Hz, 2H), 7.51 (d, J=8.8 Hz, 2H), 7.56 (d, J=8.8 Hz, 2H), 8.33 (s, 1H), 8.84-8.87 (m, 2H).

### Example 187

### Synthesis of 3-(1,3-bis(4-chlorophenyl)-5-cyclopropyl-4-hydroxy-2-oxoimidazolidin-4-yl)benzonitrile, 3-((5S)-1,3-bis(4-chlorophenyl)-5-cyclopropyl-4-hydroxy-2-oxoimidazolidin-4-yl)benzonitrile, and 3-((5R)-1,3-bis(4-chlorophenyl)-5-cyclopropyl-4-hydroxy-2-oxoimidazolidin-4-yl)benzonitrile

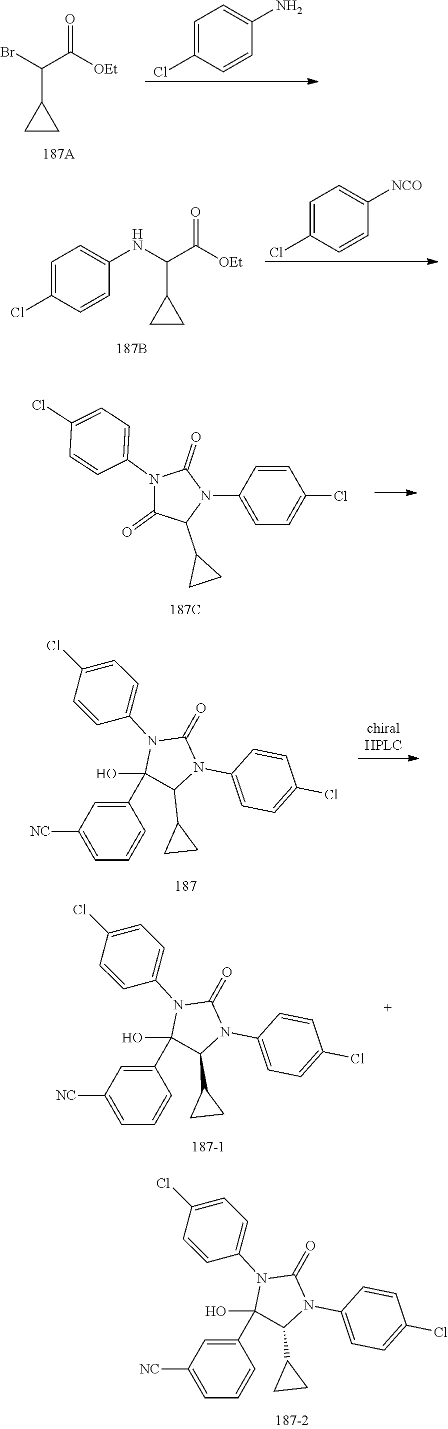

A mixture of ethyl 2-bromo-2-cyclopropylacetate 187A (3.0 g, 14.5 mmol), 4-chloroaniline (1.8 g, 14.5 mmol), and NaHCO₃ (3.7 g, 43.5 mmol) in ethanol (100 mL) was stirred at 50° C. for 16 hours. The mixture was evaporated under reduced pressure. The residue was purified with flash column chromatography on silica gel (ethyl acetate in petroleum ether, 20% v/v) to give Compound 187B. LC-MS (ESI) m/z: 254 [M+H]⁺; ¹H-NMR (CDCl₃, 400 MHz): δ (ppm) 0.35-0.59 (m, 4H), 0.85-0.87 (m, 1H), 1.26 (t, J=7.2 Hz, 3H), 1.55 (t, J=7.2 Hz, 1H), 4.18-4.26 (m, 3H), 6.51 (d, J=6.8 Hz, 2H), 7.10 (d, J=6.8 Hz, 2H).

Compounds 187C and 187 were synthesized by employing the procedures described for Compounds 128C and 125 using Compounds 187B at 50° C., 187C, and 3-bromobenzonitrile in lieu of Compounds 128B at room temperature, 2C, and 3-bromo-N,N-dimethylaniline. Compound 187C. LC-MS (ESI) m/z: 361 [M+H]⁺; ¹H-NMR (CDCl₃, 400 MHz): δ (ppm) 0.48-0.65 (m, 4H), 1.16-1.18 (m, 1H), 4.25 (d, J=8.0 Hz, 1H), 7.40-7.47 (m, 8H). Compound 187. LC-MS (ESI) m/z: 464 [M+H]⁺; ¹H-NMR (CDCl₃, 400 MHz): δ (ppm) −0.39-−0.08 (m, 1H), 0.43-0.68 (m, 3H), 0.92-1.20 (m, 1H), 2.01, 4.09 (s, 1H), 3.23, 4.24 (d, J=9.2 Hz, 1H), 7.08-7.24 (m, 5H), 7.28-7.48 (m, 7H).

Compound 187 was separated by using chiral HPLC to yield Compound 187-1 and Compound 187-2. Compound 187-1: LC-MS (ESI) m/z: 464 [M+H]⁺; ¹H-NMR (CDCl₃, 400 MHz): δ (ppm) −0.48-0.15 (m, 2H), 0.12-0.50 (m, 2H), 0.94-1.03 (m, 1H), 3.12, 3.35 (d, J=9.2 Hz, 1H), 4.78, 5.52 (s, 1H), 7.07 (t, J=8.8 Hz, 2H), 7.16-7.24 (m, 4H), 7.30-7.41 (m, 3H), 7.52-7.68 (m, 2H), 7.76, 7.88 (s, 1H). Chiral separation conditions: MeOH contained 0.2% Methanol Ammonia; IC (4.6*100 mm, 5 μm); retention time: 1.63 minutes (65%), 1.78 minutes (26%). Compound 187-2: LC-MS (ESI) m/z: 464 [M+H]⁺; ¹H-NMR (CDCl₃, 400 MHz): δ (ppm) −0.44-−0.12 (m, 2H), 0.15-0.51 (m, 2H), 0.94-1.03 (m, 1H), 3.16, 3.37 (d, J=9.6 Hz, 1H), 4.44, 5.10 (s, 1H), 7.10-7.25 (m, 6H), 7.27-7.43 (m, 3H), 7.54-7.69 (m, 2H), 7.78, 7.89 (s, 1H). Chiral separation conditions: MeOH contained 0.2% Methanol Ammonia; IC (4.6*100 mm, 5 μm); retention time: 1.40 minutes (33%), 2.88 minutes (62%).

### Example 188

### Synthesis of 5-(1,3-bis(4-chlorophenyl)-4-hydroxy-5,5-dimethyl-2-oxoimidazolidin-4-yl)isophthalonitrile

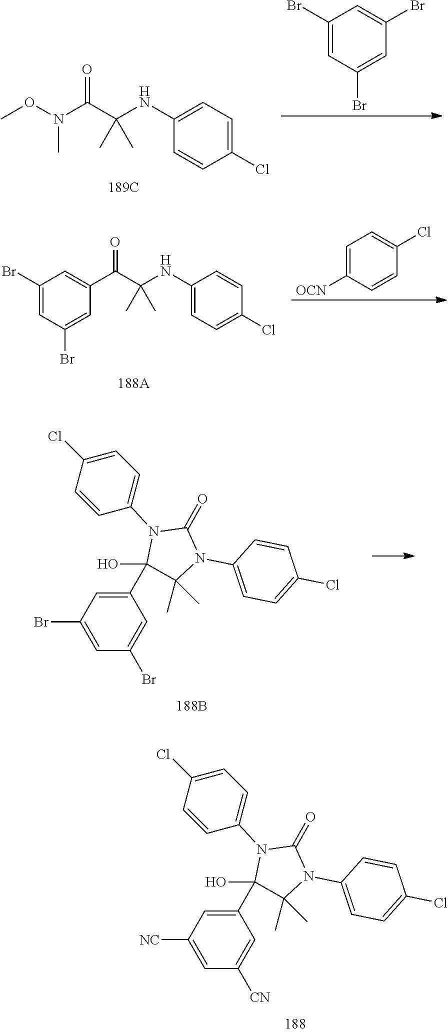

Compounds 188A, 188B, and 188 were synthesized by employing the procedures described for Compounds 59B, 128C, and 180C using Compounds 189C, 1,3,5-tribromobenzene, 186A, and 186B in lieu of N-methoxy-N-methylacetamide, Compounds 59A, 128B, and 180B. Compound 188A. LC-MS (ESI) m/z: 430 [M+H]⁺; ¹H-NMR (CDCl₃, 400 MHz): δ (ppm) 1.58 (s, 6H), 4.02 (s, 1H), 6.39 (d, J=8.8 Hz, 2H), 7.05 (d, J=8.8 Hz, 2H), 7.77 (t, J=2.0 Hz, 1H), 8.33 (d, J=2.0 Hz, 2H). Compound 188B. LC-MS (ESI) m/z: 583 [M+H]⁺; ¹H-NMR (CDCl₃, 400 MHz): δ (ppm) 0.87 (s, 3H), 1.35 (s, 3H), 3.67 (s, 1H), 7.18-7.23 (m, 4H), 7.26-7.40 (m, 4H), 7.51 (s, 2H), 7.62 (s, 1H). Compound 188. LC-MS (ESI) m/z: 477 [M+H]⁺; ¹H-NMR (CDCl₃, 400 MHz): δ (ppm) 0.80 (s, 3H), 1.31 (s, 3H), 4.87 (d, J=12.4 Hz, 1H), 7.10-7.22 (m, 5H), 7.31-7.37 (m, 5H), 7.87 (s, 1H).

### Example 189

### Synthesis of 1,3-bis(4-chlorophenyl)-4-hydroxy-5,5-dimethyl-4-(pyridin-3-yl)imidazolidin-2-one

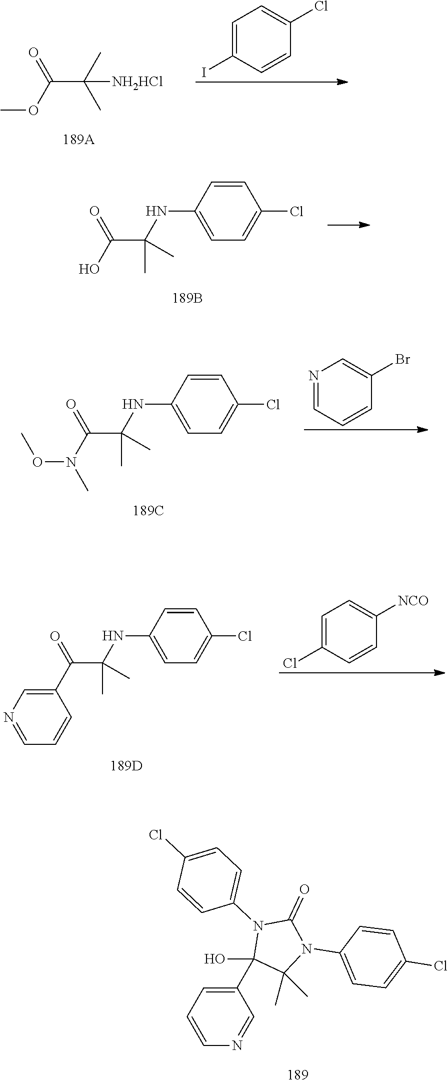

Compounds 189B, 189C, 189D, and 189 were synthesized by employing the procedures described for Compounds 224B, 98B, 59B, and 128C using Compounds 189A, 189B, 189C, 3-bromopyridine, and 189D at 80° C. in lieu of 1-isocyanato-4-methoxybenzene, Compounds 224A, 98A, N-methoxy-N-methylacetamide, 59A, and 128B at room temperature. Compound 189B. LC-MS (ESI) m/z: 214 [M+H]⁺; ¹H-NMR (CDCl₃, 400 MHz): δ (ppm) 1.56 (s, 6H), 6.57 (d, J=8.4 Hz, 2H), 7.14 (d, J=8.4 Hz, 2H). Compound 189C. LC-MS (ESI) m/z: 257 [M+H]⁺; ¹H-NMR (CDCl₃, 400 MHz): δ (ppm) 1.56 (s, 6H), 3.21 (s, 3H), 3.51 (s, 3H), 4.10 (s, 1H), 6.50 (d, J=6.0 Hz, 2H), 7.08 (d, J=6.0 Hz, 2H). Compound 189D. LC-MS (ESI) m/z: 275 [M+H]⁺. Compound 189. LC-MS (ESI) m/z: 428 [M+H]⁺; ¹H-NMR (CDCl₃, 400 MHz): δ (ppm) 0.78 (s, 3H), 1.30 (s, 3H), 5.13 (s, 1H), 7.06-7.14 (m, 4H), 7.24-7.25 (m, 1H), 7.32-7.39 (m, 4H), 7.66-7.68 (m, 1H), 8.52-8.33 (m, 2H).

### Example 190

### Synthesis of 1,3-bis(4-chlorophenyl)-5-hydroxy-4-methyl-4-(3-(trifluoromethyl)phenyl)imidazolidin-2-one, (4S)-1,3-bis(4-chlorophenyl)-5-hydroxy-4-methyl-4-(3-(trifluoromethyl)phenyl)imidazolidin-2-one, and (4R)-1,3-bis(4-chlorophenyl)-5-hydroxy-4-methyl-4-(3-(trifluoromethyl)phenyl)imidazolidin-2-one

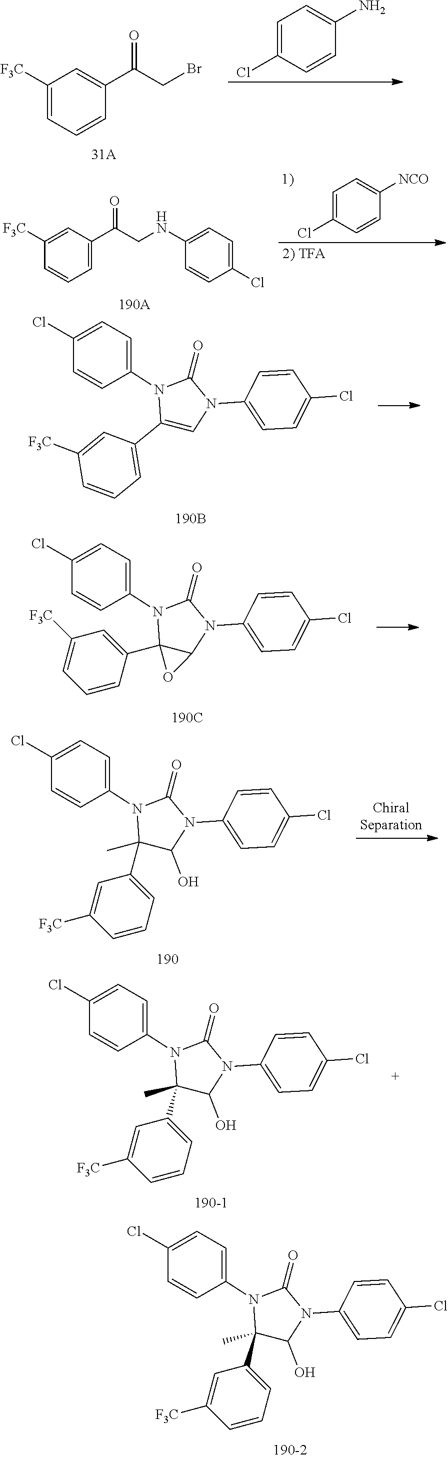

Compounds 190A, 190B, 190C, and 190 were synthesized by employing the procedures described for Compounds 1B, 86A, 86B, and 86 using Compounds 31A, 4-chloroaniline, 190A, 1-chloro-4-isocyanatobenzene, 190B, and 190C in lieu of Compounds 1A, 4-bromoaniline, 1B, 1-bromo-4-isocyanatobenzene, 86A, and 86B. Compound 190A. LC-MS (ESI) m/z: 314 [M+H]⁺; ¹H-NMR (CDCl₃, 400 MHz): δ (ppm) 4.62 (d, J=4.8 Hz, 2H), 4.91 (s, 1H), 6.63-6.67 (m, 2H), 7.16-7.20 (m, 2H), 7.69 (t, J=8.0 Hz, 1H), 7.90 (d, J=7.6 Hz, 1H), 8.20 (t, J=7.6 Hz, 1H), 8.26 (s, 1H). Compound 190B. LC-MS (ESI) m/z: 449 [M+H]⁺. Compound 190C. LC-MS (ESI) m/z: 465 [M+H]⁺; ¹H-NMR (CDCl₃, 400 MHz): δ (ppm) 5.66 (s, 1H), 7.29-7.33 (m, 2H), 7.41-7.48 (m, 6H), 7.53-7.66 (m, 4H). Compound 190. LC-MS (ESI) m/z: 481 [M+H]⁺; ¹H-NMR (CDCl₃, 400 MHz): δ (ppm) 0.89 (s, 3H), 4.32 (s, 1H), 5.11 (s, 1H), 7.13-7.15 (m, 2H), 7.23-7.37 (m, 7H), 7.45-7.49 (m, 2H), 7.57-7.59 (m, 1H).

Compound 190 was separated by using chiral HPLC to yield Compound 190-1 and Compound 190-2. Compound 190-1: LC-MS (ESI) m/z: 481 [M+H]⁺; ¹H-NMR (CDCl₃, 400 MHz): δ (ppm) 0.91 (s, 0.3H), 1.63 (s, 2.7H), 4.20 (brs, 1H), 5.13 (s, 0.1H), 5.20 (s, 0.9H), 7.13-7.16 (m, 2H), 7.25-7.38 (m, 6H), 7.46-7.59 (m, 4H). Chiral separation conditions: MeOH contained 0.2% Methanol Ammonia; OZ-H (250*4.6 mm, 5 μm); retention time: 3.7 minutes (10.3%), 4.29 minutes (89.7%). Compound 190-2: LC-MS (ESI) m/z: 481 [M+H]⁺; ¹H-NMR (CDCl₃, 400 MHz): δ (ppm) 0.93 (s, 1.4H), 1.62 (s, 1.6H), 3.95 (brs, 1H), 5.14 (s, 0.46H), 5.21 (s, 0.54H), 7.15-7.18 (m, 3H), 7.21-7.39 (m, 6H), 7.47-7.64 (m, 3H); Chiral separation conditions: MeOH contained 0.2% Methanol Ammonia; OZ-H (250*4.6 mm, 5 μm); retention time: 6.2 minutes (53.4%), 8.96 minutes (46.6%).

### Example 191

### Synthesis of 1,3-bis(4-chlorophenyl)-5-cyclobutyl-4-hydroxy-4-(3-(trifluoromethoxy)phenyl)imidazolidin-2-one, (5S)-1,3-bis(4-chlorophenyl)-5-cyclobutyl-4-hydroxy-4-(3-(trifluoromethoxy)phenyl)imidazolidin-2-one, and (5R)-1,3-bis(4-chlorophenyl)-5-cyclobutyl-4-hydroxy-4-(3-(trifluoromethoxy)phenyl)imidazolidin-2-one

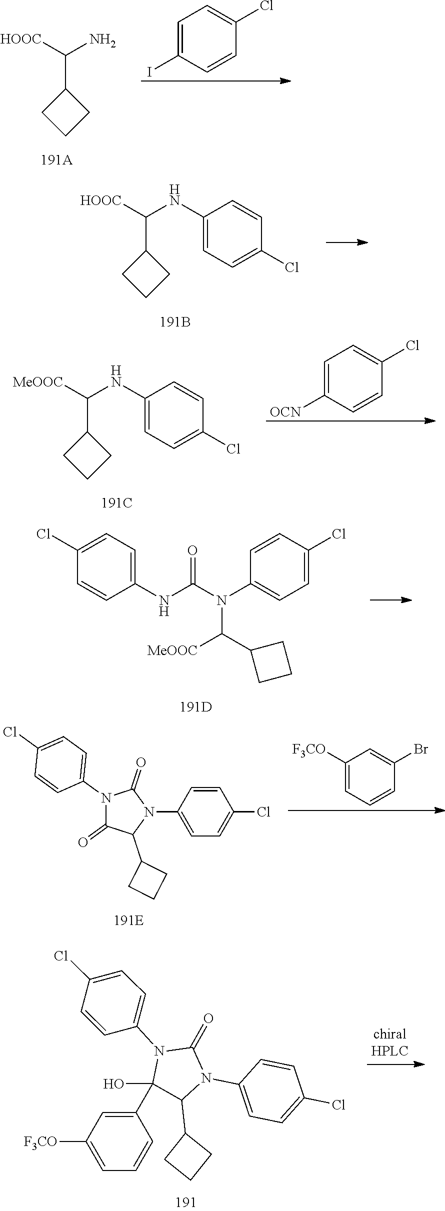

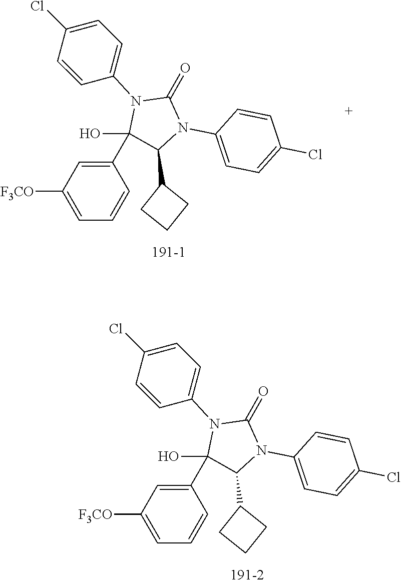 [SMILES: O=C1N(c2ccc(Cl)cc2)[C@@H](C2CCC2)C(O)(c2cccc(OC(F)(F)F)c2)N1c1ccc(Cl)cc1 ; O=C1N(c2ccc(Cl)cc2)[C@H](C2CCC2)C(O)(c2cccc(OC(F)(F)F)c2)N1c1ccc(Cl)cc1]

Compounds 191B, 191C, 191D, 191E, and 191 were synthesized by employing the procedures described for Compounds 224B, 224C, 224D, 224E, and 125 using Compounds 191A in the presence of 2-acetylcyclohexan-1-one, 191B, 191C, 191D, 191E, and 1-bromo-3-(trifluoromethoxy)benzene in lieu of Compounds 224A without 2-acetylcyclohexan-1-one, 224B, 224C, 224D, 2C, and 3-bromo-N,N-dimethylaniline. Compound 191B. LC-MS (ESI) m/z: 240 [M+H]⁺. Compound 191C. LC-MS (ESI) m/z: 254 [M+H]⁺; ¹H-NMR (CDCl₃, 400 MHz): δ (ppm) 1.83-2.10 (m, 6H), 2.62-2.72 (m, 1H), 3.69 (s, 3H), 3.93 (t, J=8.4 Hz, 1H), 4.02 (d, J=8.8 Hz, 1H), 6.53 (d, J=8.0 Hz, 2H), 7.11 (d, J=8.0 Hz, 2H). Compound 191D. LC-MS (ESI) m/z: 407 [M+H]⁺; ¹H-NMR (CDCl₃, 400 MHz): δ (ppm) 1.79-1.97 (m, 6H), 2.44-2.50 (m, 1H), 3.76 (s, 3H), 4.87 (d, J=10.8 Hz, 1H), 5.98 (s, 1H), 7.17-7.22 (m, 3H), 7.40-7.47 (m, 5H). Compound 191E. LC-MS (ESI) m/z: 375 [M+H]⁺; ¹H-NMR (CDCl₃, 400 MHz): δ (ppm) 1.74-1.97 (m, 5H), 2.22-2.32 (m, 1H), 2.84-2.92 (m, 1H), 4.60 (d, J=5.6 Hz, 1H), 7.38-7.52 (m, 8H). Compound 191. LC-MS (ESI) m/z: 537 [M+H]⁺; ¹H-NMR (CD₃OD, 400 MHz): δ (ppm) 1.24-1.40 (m, 2H), 1.45-1.93 (m, 4H), 2.23-2.31, 2.79-2.90 (m, 1H), 4.37-4.41 (m, 1H), 7.15-7.22 (m, 3H), 7.35-7.49 (m, 7H), 7.55-7.65 (m, 2H).

Compound 191 was separated by using chiral HPLC to give Compound 191-1 and Compound 191-2. Compound 191-1: LC-MS (ESI) m/z: 537 [M+H]⁺; ¹H-NMR (CDCl₃, 400 MHz): δ (ppm) 1.23-1.55 (m, 4H), 1.73-1.93 (m, 2H), 2.69-2.80 (m, 1H), 4.16, 5.08 (s, 1H), 4.18 (d, J=3.2 Hz, 1H), 7.03-7.07 (m, 2H), 7.11-7.42 (m, 10H). Chiral separation conditions: MeOH contained 0.2% Methanol Ammonia; Enantiopark-OD (4.6*100 mm, 5 μm); retention time: 1.1 minutes (92.8%), 2.54 minutes (6%). Compound 191-2: LC-MS (ESI) m/z: 537 [M+H]⁺; ¹H-NMR (CDCl₃, 400 MHz): δ (ppm) 0.25-0.31 (m, 1H), 1.19-1.40 (m, 5H), 2.08-2.19, 2.68-2.74 (m, 1H), 4.06, 4.65 (s, 1H), 4.12, 4.18 (d, J=9.6 Hz, 1H), 7.08-7.25 (m, 5H), 7.27-7.43 (m, 7H). Chiral separation conditions: MeOH contained 0.2% Methanol Ammonia; Enantiopark-OD (4.6*100 mm, 5 μm); retention time: 1.36 minutes (79%), 2.9 minutes (20%).

### Example 192

### Synthesis of 3-(4-hydroxy-5,5-dimethyl-2-oxo-1,3-bis(4-(trifluoromethyl)phenyl)-imidazolidin-4-yl)benzonitrile

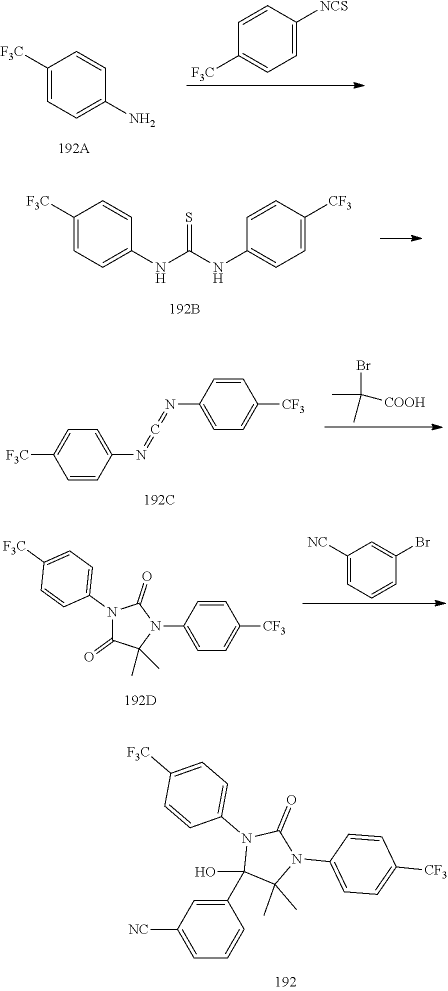

Compounds 192B, 192C, 192D, and 192 were synthesized by employing the procedures described for Compounds 120B, 120C, 120D, and 125 using 1-isothiocyanato-4-(trifluoromethyl)benzene, Compounds 192A, 192B, 192C, 3-bromobenzonitrile, and 192D in lieu of 1-chloro-4-isothiocyanatobenzene, Compounds 120A, 120B, 120C, 3-bromo-N,N-dimethylaniline, and 2C. Compound 192B. LC-MS (ESI) m/z: 365 [M+H]⁺; ¹H-NMR (DMSO-d₆, 400 MHz): δ (ppm) 7.69-7.77 (m, 8H), 10.37 (s, 2H). Compound 192C. ¹H-NMR (CDCl₃, 400 MHz): δ (ppm) 7.28 (d, J=8.4 Hz, 4H), 7.61 (d, J=8.4 Hz, 4H). Compound 192D. LC-MS (ESI) m/z: 417 [M+H]⁺; ¹H-NMR (CD₃OD, 400 MHz): δ (ppm) 1.61 (s, 6H), 7.68-7.70 (m, 2H), 7.77-7.79 (m, 2H), 7.83-7.85 (m, 4H). Compound 192. LC-MS (ESI) m/z: 520 [M+H]⁺. ¹H-NMR (DMSO-d₆, 400 MHz): δ (ppm) 0.83 (s, 3H), 1.31 (s, 3H), 7.53 (s, 1H), 7.61-7.73 (m, 7H), 7.79-7.88 (m, 5H).

### Example 193

### Synthesis of 1,3-bis(4-bromophenyl)-5-hydroxy-5-methylimidazolidine-2,4-dione

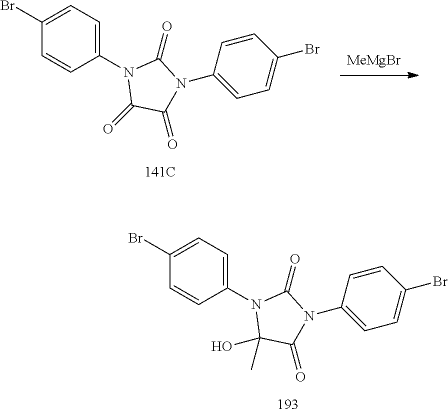 [SMILES: "MeMgBr" ; O=C1C(=O)N(c2ccc(Br)cc2)C(=O)N1c1ccc(Br)cc1 ; CC1(O)C(=O)N(c2ccc(Br)cc2)C(=O)N1c1ccc(Br)cc1]

Compound 193 was synthesized by employing the procedure described for Compound 141 using methylmagnesium bromide in lieu of phenylmagnesium bromide. LC-MS (ESI) m/z: 439 [M+H]⁺; ¹H-NMR (DMSO-d₆, 400 MHz): δ (ppm) 1.47 (s, 3H), 7.47-7.52 (m, 5H), 7.67-7.76 (m, 4H).

### Example 194

### Synthesis of 3-(1,3-bis(4-chlorophenyl)-5-cyclobutyl-4-hydroxy-2-oxoimidazolidin-4-yl)benzonitrile, 3-((5S)-1,3-bis(4-chlorophenyl)-5-cyclobutyl-4-hydroxy-2-oxoimidazolidin-4-yl)benzonitrile, and 3-((5R)-1,3-bis(4-chlorophenyl)-5-cyclobutyl-4-hydroxy-2-oxoimidazolidin-4-yl)benzonitrile

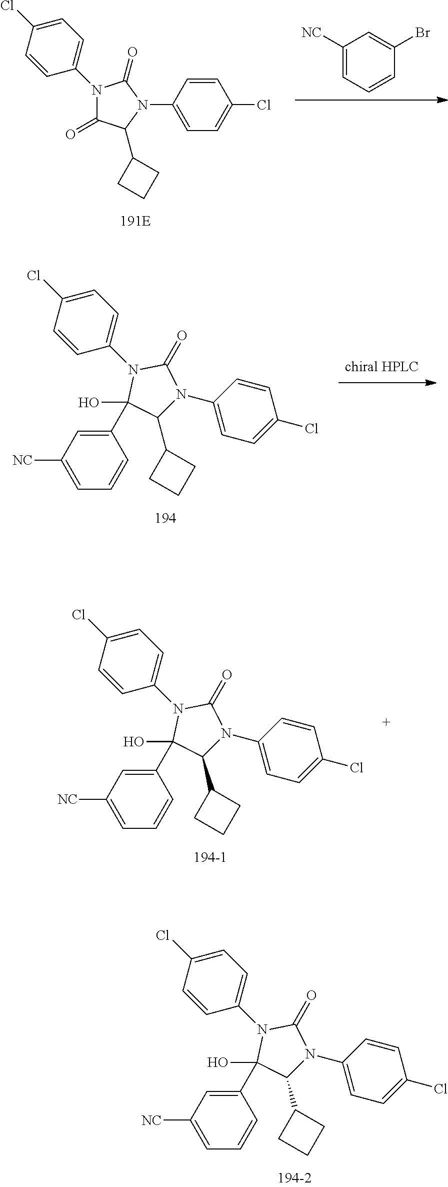 [SMILES: O=C1C(C2CCC2)N(c2ccc(Cl)cc2)C(=O)N1c1ccc(Cl)cc1 ; N#Cc1cccc(Br)c1 ; N#Cc1cccc(C2(O)C(C3CCC3)N(c3ccc(Cl)cc3)C(=O)N2c2ccc(Cl)cc2)c1 ; N#Cc1cccc(C2(O)[C@H](C3CCC3)N(c3ccc(Cl)cc3)C(=O)N2c2ccc(Cl)cc2)c1 ; N#Cc1cccc(C2(O)[C@@H](C3CCC3)N(c3ccc(Cl)cc3)C(=O)N2c2ccc(Cl)cc2)c1]

Compound 194 was synthesized by employing the procedure described for Compound 125 using Compound 191E and 3-bromobenzonitrile in lieu of Compounds 2C and 3-bromo-N,N-dimethylaniline. LC-MS (ESI) m/z: 478 [M+H]; ¹H-NMR (CD₃OD, 400 MHz): δ (ppm) 1.28-1.92 (m, 6H), 2.26-2.32, 2.79-2.90 (m, 1H), 4.40 (d, J=9.6 Hz, 1H), 7.17-7.22 (m, 2H), 7.36-7.58 (m, 7H), 7.63-8.05 (m, 3H).

Compound 194 was separated by using chiral HPLC to give Compound 194-1 and Compound 194-2. Compound 194-1: LC-MS (ESI) m/z: 478 [M+H]⁺; ¹H-NMR (CDCl₃, 400 MHz): δ (ppm) 1.18-1.56 (m, 4H), 1.74-1.93 (m, 2H), 2.10-2.16, 2.70-2.76 (m, 1H), 4.08, 4.93 (s, 1H), 4.14, 4.20 (d, J=9.6 Hz, 1H), 7.10-7.24 (m, 4H), 7.27-7.46 (m, 5H), 7.57-7.89 (m, 3H). Chiral separation conditions: MeOH contained 0.2% Methanol Ammonia; Enantiopark-OD (4.6*100 mm, 5 μm); retention time: 1.78 minutes (69%), 4.02 minutes (31%). Compound 194-2: LC-MS (ESI) m/z: 478 [M+H]⁺; ¹H-NMR (CDCl₃, 400 MHz): δ (ppm) 1.17-1.55 (m, 4H), 1.66-1.99 (m, 2H), 2.03-2.17, 2.67-2.78 (m, 1H), 4.13-4.21, 5.00 (m, 2H), 7.09-7.25 (m, 5H), 7.28-7.88 (m, 7H). Chiral separation conditions: MeOH contained 0.2% Methanol Ammonia; Enantiopark-OD (4.6*100 mm, 5 μm); retention time: 2.07 minutes (40%), 4.79 minutes (60%).

### Example 195

### Synthesis of 1,3-bis(4-bromophenyl)-5-hydroxy-5-(naphthalen-1-yl)imidazolidine-2,4-dione

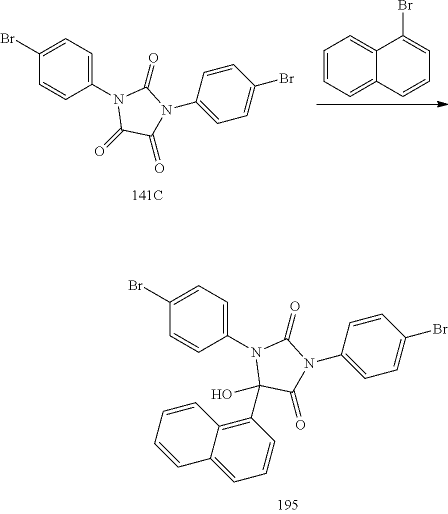

Compound 195 was synthesized by employing the procedure described for Compound 127 using Compound 141C and 1-bromonaphthalenein lieu of Compound 2C and 1-bromo-4-(tert-butyl)benzene. LC-MS (ESI) m/z: 1123[2M+Na]⁺; ¹H-NMR (CDCl₃, 400 MHz): δ (ppm) 7.08-7.10 (m, 2H), 7.23-7.25 (m, 2H), 7.26 (s, 1H), 7.43-7.45 (m, 3H), 7.51-7.54 (m, 2H), 7.64 (d, J=8.4 Hz, 2H), 7.87-7.91 (m, 2H), 7.96-7.97 (m, 1H).

### Example 196

### Synthesis of 4-hydroxy-5-isopropyl-4-(3-(trifluoromethoxy)phenyl)-1,3-bis(4-(trifluoromethyl)phenyl)imidazolidin-2-one, (5S)-4-hydroxy-5-isopropyl-4-(3-(trifluoromethoxy)phenyl)-1,3-bis(4-(trifluoromethyl)phenyl)imidazolidin-2-one, and (5R)-4-hydroxy-5-isopropyl-4-(3-(trifluoromethoxy)phenyl)-1,3-bis(4-(trifluoromethyl)phenyl)imidazolidin-2-one

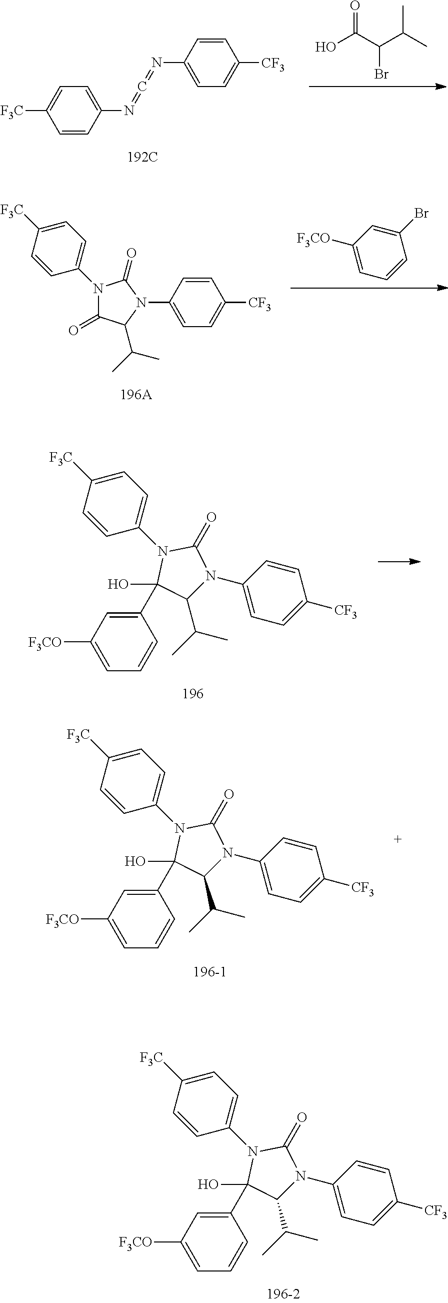

Compounds 196A and 196 were synthesized by employing the procedures described for Compounds 120D and 125 using 2-bromo-3-methylbutanoic acid, Compounds 192C using Na₂CO₃ as base and DMF as solvent, 196A, and 1-bromo-3-(trifluoromethoxy)benzene in lieu of 2-bromo-2-methylpropanoic acid, Compounds 120C using NaOH as base and 1,4-dioxane as solvent, 2C, and 3-bromo-N,N-dimethylaniline. Compound 196A. LC-MS (ESI) m/z: 431 [M+H]⁺; ¹H-NMR (CDCl₃, 400 MHz): δ (ppm) 0.94 (d, J=7.2 Hz, 3H), 1.30 (d, J=7.2 Hz, 3H), 2.33-2.41 (m, 1H), 4.71 (d, J=3.2 Hz, 1H), 7.62-7.65 (m, 4H), 7.73 (d, J=8.4 Hz, 2H), 7.78 (d, J=8.4 Hz, 2H). Compound 196. LC-MS (ESI) m/z: 593 [M+H]⁺; ¹H-NMR (CDCl₃, 400 MHz): δ (ppm) 0.57, 0.98 (d, J=7.2 Hz, 3H), 0.69, 1.04 (d, J=7.2 Hz, 3H), 1.46-1.53, 2.38-2.45 (m, 1H), 3.28, 4.10 (s, 1H), 4.31, 4.38 (d, J=3.2 Hz, 1H), 7.19-7.24 (m, 1H), 7.31-7.40 (m, 1H), 7.42-7.54 (m, 7H), 7.65 (t, J=8.4 Hz, 2H), 7.73 (d, J=8.4 Hz, 1H).

Compound 196 was separated by using chiral HPLC to give Compound 196-1 and Compound 196-2. Compound 196-1: LC-MS (ESI) m/z: 593 [M+H]⁺; ¹H-NMR (CDCl₃, 400 MHz): δ (ppm) 0.53, 0.95 (d, J=7.2 Hz, 3H), 0.66, 1.00 (d, J=7.2 Hz, 3H), 1.42-1.50, 2.34-2.42 (m, 1H), 3.77, 4.94 (s, 1H), 4.28, 4.32 (d, J=3.2 Hz, 1H), 7.20-7.22 (m, 1H), 7.38-7.65 (m, 11H); Chiral separation conditions: MeOH contained 0.2% Methanol Ammonia; EnantioPaK OD (4.6*100 mm, 5 μm); retention time: 0.50 minutes (87%), 0.76 minutes (13%). Compound 196-2: LC-MS (ESI) m/z: 593 [M+H]⁺; ¹H-NMR (CDCl₃, 400 MHz): δ (ppm) 0.52, 0.94 (d, J=7.2 Hz, 3H), 0.64, 0.98 (d, J=7.2 Hz, 3H), 1.41-1.49, 2.32-2.40 (m, 1H), 3.87, 5.10 (s, 1H), 4.27, 4.31 (d, J=2.8 Hz, 1H), 7.16-7.21 (m, 1H), 7.36-7.64 (m, 11H); Chiral separation conditions: MeOH contained 0.2% Methanol Ammonia; EnantioPaK OD (4.6*100 mm, 5 μm); retention time: 0.57 minutes (100%).

### Example 197

### Synthesis of 1,3-bis(4-bromophenyl)-5-(5-chlorothiophen-2-yl)-5-hydroxyimidazolidine-2,4-dione

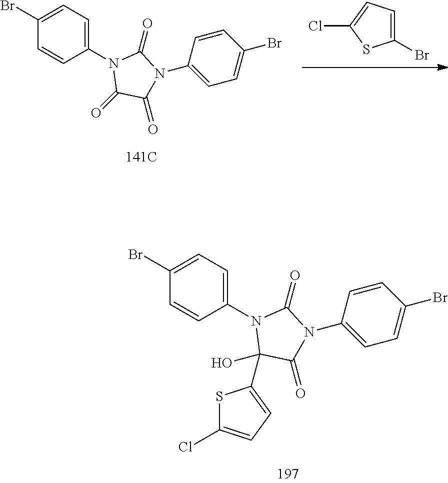 [SMILES: O=C1C(=O)N(c2ccc(Br)cc2)C(=O)N1c1ccc(Br)cc1 ; Clc1ccc(Br)s1 ; O=C1N(c2ccc(Br)cc2)C(=O)C(O)(c2ccc(Cl)s2)N1c1ccc(Br)cc1]

Compound 197 was synthesized by employing the procedure described for Compound 127 using Compound 141C and 2-bromo-5-chlorothiophene in lieu of Compound 2C and 1-bromo-4-(tert-butyl)benzene. LC-MS (ESI) m/z: 541; ¹H-NMR (CDCl₃, 400 MHz): δ (ppm) 6.86 (d, J=4.4 Hz, 1H), 6.96 (d, J=3.6 Hz, 1H), 7.43 (d, J=8.4 Hz, 2H), 7.50 (d, J=8.8 Hz, 2H), 7.57 (d, J=8.8 Hz, 2H), 7.69 (d, J=8.8 Hz, 2H).

### Example 198

### Synthesis of 1,3-bis(4-bromophenyl)-5-(furan-2-yl)-5-hydroxyimidazolidine-2,4-dione

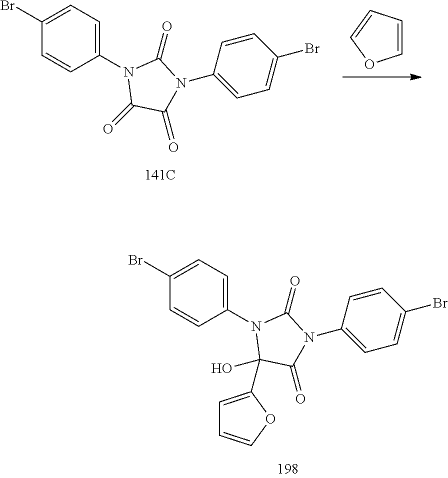

Compound 198 was synthesized by employing the procedure described for Compound 127 using Compound 141C and furan in lieu of Compound 2C and 1-bromo-4-(tert-butyl)benzene. LC-MS (ESI) m/z: 1003 [2M+Na]⁺; ¹H-NMR (CD₃OD, 400 MHz): δ (ppm) 6.29-7.32 (m, 1H), 6.55 (d, J=8.8 Hz, 1H), 7.24 (dd, J=6.8, 2.0 Hz, 2H), 7.30-7.44 (m, 5H), 7.60 (dd, J=6.8, 2.0 Hz, 2H).

### Example 199

### Synthesis of 1,3-bis(4-bromophenyl)-5-hydroxy-5-octylimidazolidine-2,4-dione

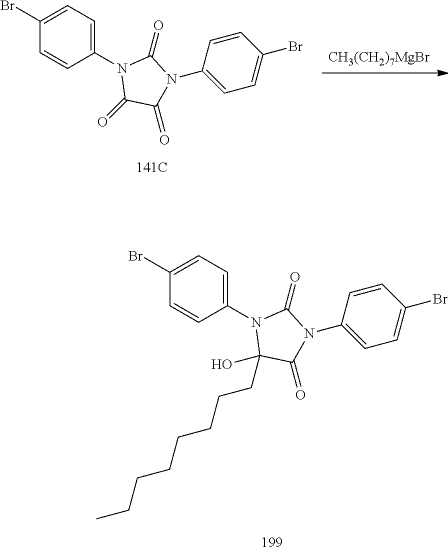

Compound 199 was synthesized by employing the procedure described for Compound 141 using octylmagnesium bromide in lieu of phenylmagnesium bromide. LC-MS (ESI) m/z: 1095 [2M+Na]⁺; ¹H-NMR (DMSO-d₆, 400 MHz): δ (ppm) 0.85 (t, J=6.8 Hz, 3H), 1.11-1.25 (m, 12H), 1.70-1.89 (m, 2H), 7.39 (d, J=8.8 Hz, 2H), 7.55 (d, J=8.8 Hz, 2H), 7.65 (s, 1H), 7.68 (d, J=8.8 Hz, 2H), 7.75 (d, J=8.8 Hz, 2H).

### Example 200

### Synthesis of 1,3-bis(4-bromophenyl)-5-hydroxy-5-(p-tolyl)imidazolidine-2,4-dione

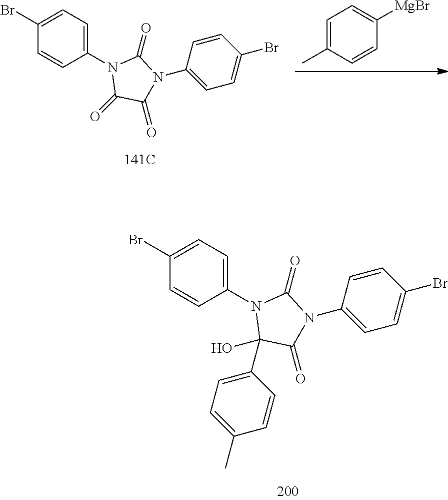

Compound 200 was synthesized by employing the procedure described for Compound 141 using p-tolylmagnesium bromide in lieu of phenylmagnesium bromide. LC-MS (ESI) m/z: 515 [M+H]⁺; ¹H-NMR (CD₃OD, 400 MHz): δ (ppm) 2.32 (s, 3H), 7.20 (d, J=8.0 Hz, 2H), 7.40-7.51 (m, 8H), 7.70 (d, J=8.8 Hz, 2H).

### Example 201

### Synthesis of 1,3-bis(4-bromophenyl)-5-hydroxy-5-(thiazol-2-yl)imidazolidine-2,4-dione

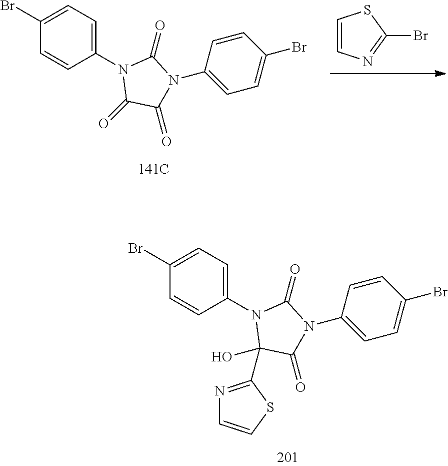

Compound 201 was synthesized by employing the procedure described for Compound 127 using Compound 141C and 2-bromothiazole in lieu of Compound 2C and 1-bromo-4-(tert-butyl)benzene. LC-MS (ESI) m/z: 508 [M+H]⁺; ¹H-NMR (CD₃OD, 400 MHz): δ (ppm) 7.28 (d, J=8.8 Hz, 2H), 7.36 (d, J=8.8 Hz, 4H), 7.53 (d, J=3.2 Hz, 1H), 7.61 (d, J=8.8 Hz, 2H), 7.73 (d, J=3.2 Hz, 1H).

### Example 202

### Synthesis of 1,3-bis(4-bromophenyl)-5-hydroxy-5-(6-methoxypyridin-3-yl)imidazolidine-2,4-dione

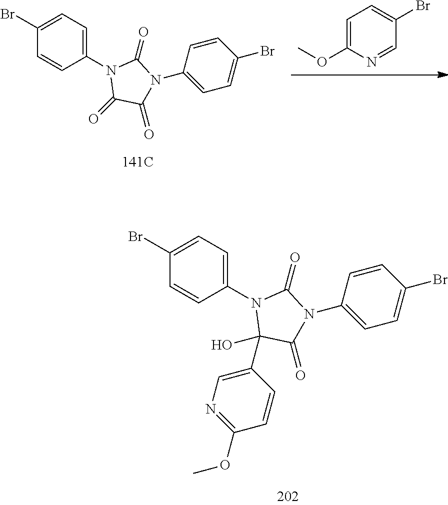

Compound 202 was synthesized by employing the procedure described for Compound 127 using Compound 141C and 5-bromo-2-methoxypyridine in lieu of Compound 2C and 1-bromo-4-(tert-butyl)benzene. LC-MS (ESI) m/z: 532 [M+H]⁺; ¹H-NMR (CDCl₃, 400 MHz): δ (ppm) 3.89 (s, 3H), 5.76 (s, 1H), 6.71 (d, J=8.8 Hz, 1H), 7.10-7.13 (m, 2H), 7.32-7.38 (m, 4H), 7.47-7.50 (m, 2H), 7.63 (dd, J=8.8 Hz, 1H), 8.22 (d, J=2.4 Hz, 1H).

### Example 203

### Synthesis of 5-hydroxy-1,3-bis(4-iodophenyl)-5-phenylimidazolidine-2,4-dione

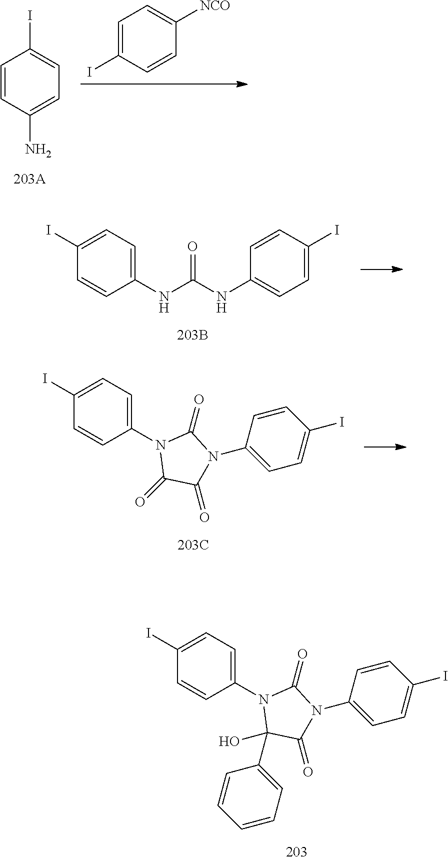

Compounds 203B, 203C, and 203 were synthesized by employing the procedures described for Compounds 160B, 141C, and 141 using Compounds 203A, 1-iodo-4-isocyanatobenzene, 203B, and 203C in lieu of Compounds 160A, 1-isocyanato-4-methoxybenzene, 141B, and 141C. Compound 203B. LC-MS (ESI) m/z: 465 [M+H]⁺; ¹H-NMR (DMSO-d₆, 400 MHz): δ (ppm) 7.29 (d, J=8.8 Hz, 4H), 7.60 (d, J=8.8 Hz, 4H), 8.83 (s, 2H). Compound 203C. LC-MS (ESI) m/z: non-ionizable compound under routine conditions used; ¹H-NMR (DMSO-d₆, 400 MHz): δ (ppm) 7.25 (d, J=8.8 Hz, 4H), 7.95 (d, J=8.8 Hz, 4H). Compound 203. LC-MS (ESI) m/z: 597 [M+H]⁺; ¹H-NMR (CDCl₃, 400 MHz): δ (ppm) 5.12 (s, 1H), 7.03 (d, J=8.8 Hz, 2H), 7.13 (d, J=8.8 Hz, 2H), 7.37-7.40 (m, 3H), 7.44-7.46 (m, 2H), 7.53 (d, J=8.8 Hz, 2H), 7.72 (d, J=8.4 Hz, 2H).

### Example 204

### Synthesis of tert-butyl 2-(1,3-bis(4-bromophenyl)-4-hydroxy-2,5-dioxoimidazolidin-4-yl)-1H-pyrrole-1-carboxylate

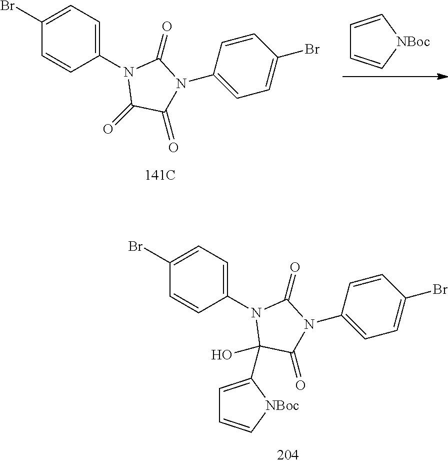

To a solution of 2,2,6,6-tetramethylpiperidine (35 mg, 0.25 mmol) in dry THF (10 mL) was dropped a solution of n-BuLi in THF (2.5 M, 0.1 mL, 0.25 mmol) at −78° C. and stirred at the same temperature for 30 minutes. To the mixture was added a solution of tert-butyl 1H-pyrrole-1-carboxylate (50 mg, 0.25 mmol) in THF (1 mL) at −78° C. and stirred at the same temperature for 30 minutes. To above mixture was dropped a solution of Compound 141C (106 mg, 0.25 mmol) in THF (2 mL) at −78° C. After stirred at) at −78° C. for 30 minutes, the reaction mixture was quenched with saturated aqueous ammonium chloride solution (50 mL) and extracted with ethyl acetate (50 mL×3). The combined organic layers was washed with brine (100 mL), dried over anhydrous sodium sulfate, filtered, and concentrated to give a crude product, which was purified with preparative HPLC to furnish Compound 204. LC-MS (ESI) m/z: 592 [M+H]⁺; ¹H-NMR (CDCl₃, 400 MHz): δ (ppm) 1.56 (s, 9H), 6.09-6.11 (m, 1H), 6.79-6.80 (m, 1H), 7.12-7.13 (m, 1H), 7.22 (d, J=8.8 Hz, 2H), 7.37 (d, J=8.8 Hz, 2H), 7.43 (d, J=8.8 Hz, 2H), 7.49-7.50 (m, 1H), 7.63 (d, J=8.8 Hz, 2H).

### Example 205

### Synthesis of 1,3-bis(4-bromophenyl)-5-hydroxy-5-(thiophen-2-yl)imidazolidine-2,4-dione

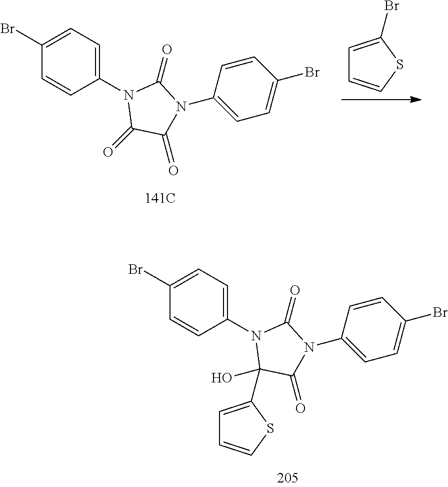

Compound 205 was synthesized by employing the procedure described for Compound 127 using Compound 141C and 2-bromothiophenein lieu of Compound 2C and 1-bromo-4-(tert-butyl)benzene. LC-MS (ESI) m/z: 507; ¹H-NMR (CDCl₃, 400 MHz): δ (ppm) 6.97-6.99 (m, 1H), 7.00-7.13 (m, 1H), 7.35-7.39 (m, 3H), 7.42-7.46 (m, 4H), 7.47 (d, J=6.4 Hz, 2H).

### Example 206

### Synthesis of 1,3-bis(4-bromophenyl)-5-hydroxy-5-(2-methoxyphenyl)imidazolidine-2,4-dione

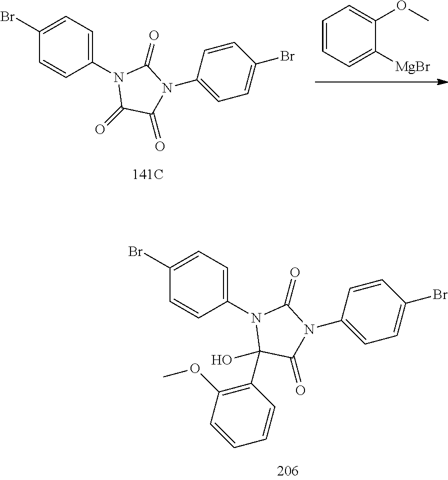

Compound 206 was synthesized by employing the procedure described for Compound 141 using (2-methoxyphenyl)magnesium bromide in lieu of phenylmagnesium bromide. LC-MS (ESI) m/z: 531 [M+H]⁺; 1H-NMR (CD3OD, 400 MHz): δ (ppm) 3.83 (s, 3H), 6.92-6.97 (m, 2H), 7.27-7.38 (m, 5H), 7.46-7.50 (m, 2H), 7.70-7.75 (m, 3H).

### Example 207

### Synthesis of 1,3-bis(4-bromophenyl)-5-hydroxy-5-(tetrahydro-2H-pyran-4-yl)imidazolidine-2,4-dione

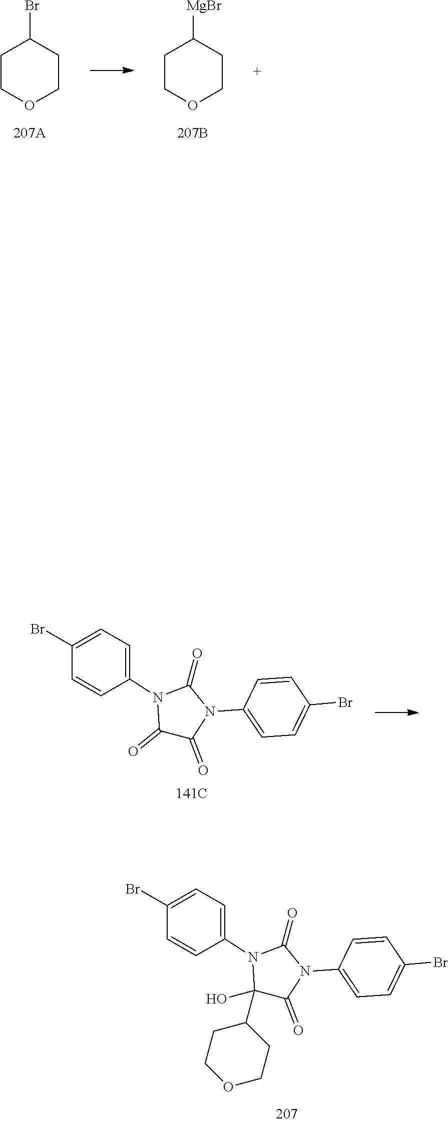

Compounds 207B and 207 were synthesized by employing the procedures described for Compounds 174B and 141 using Compounds 207A, 207B, and at −60° C. in lieu of Compounds 174A, phenylmagnesium bromide, and −78° C.

Compound 207B as a Grignard reagent solution, which was directly used in the next step. Compound 207. LC-MS (ESI) m/z: 509 [M+H]⁺; ¹H-NMR (DMSO-d₆, 400 MHz): δ (ppm) 1.02-1.12 (m, 1H), 1.54-1.65 (m, 2H), 1.75-1.86 (m, 1H), 1.94-2.01 (m, 1H), 304-3.18 (m, 2H), 3.77-3.86 (m, 2H), 3.39 (d, J=8.4 Hz, 2H), 7.53 (d, J=8.8 Hz, 2H), 7.68-7.79 (m, 5H).

### Example 208

### Synthesis of 1,3-bis(4-bromophenyl)-5-hydroxy-5-(5-methoxypyridin-2-yl)imidazolidine-2,4-dione

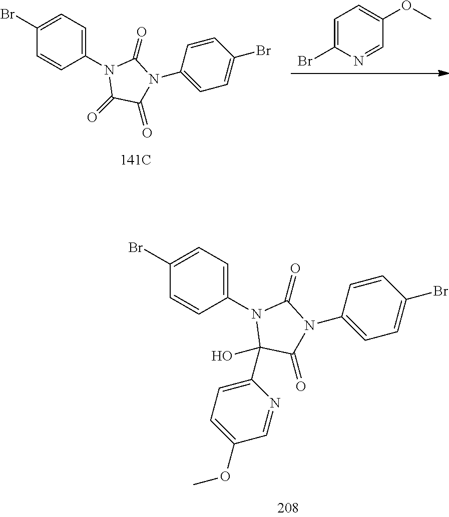

Compound 208 was synthesized by employing the procedure described for Compound 127 using Compound 141C and 2-bromo-5-methoxypyridine lieu of Compound 2C and 1-bromo-4-(tert-butyl)benzene. LC-MS (ESI) m/z: 532 [M+H]⁺; ¹H-NMR (CDCl₃, 400 MHz): δ (ppm) 3.88 (s, 3H), 6.98 (br, 1H), 7.26-7.29 (m, 3H), 7.34-7.45 (m, 5H), 7.61-7.64 (m, 2H), 8.24 (d, J=4.9 Hz, 1H).

### Example 209

### Synthesis of 1,3-bis(4-bromophenyl)-4-cyclopropyl-5-hydroxy-4-phenylimidazolidin-2-one, (4S)-1,3-bis(4-bromophenyl)-4-cyclopropyl-5-hydroxy-4-phenylimidazolidin-2-one, and (4R)-1,3-bis(4-bromophenyl)-4-cyclopropyl-5-hydroxy-4-phenylimidazolidin-2-one

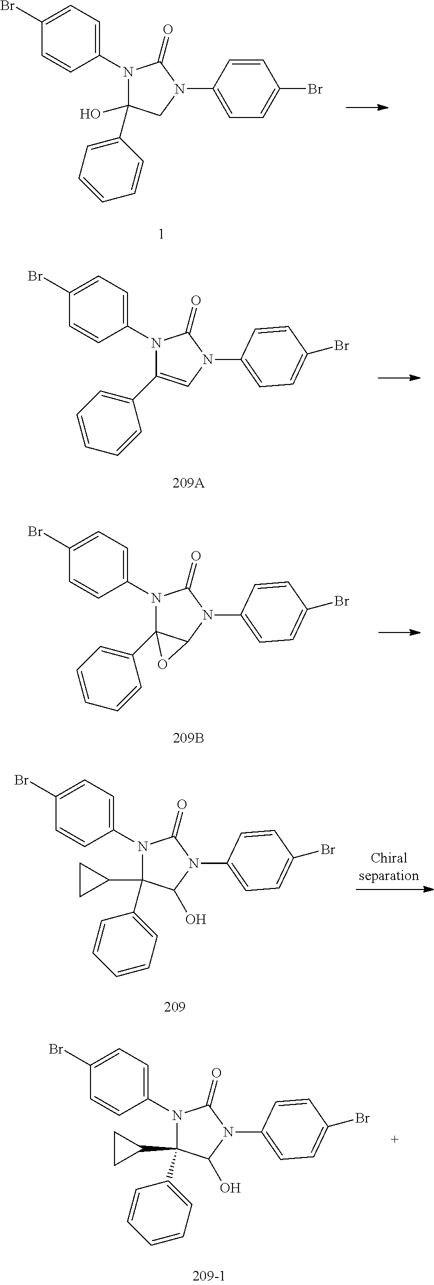 [SMILES: O=C1N(c2ccc(Br)cc2)CC(O)(c2ccccc2)N1c1ccc(Br)cc1 ; O=c1n(-c2ccc(Br)cc2)cc(-c2ccccc2)n1-c1ccc(Br)cc1 ; O=C1N(c2ccc(Br)cc2)C2OC2(c2ccccc2)N1c1ccc(Br)cc1 ; O=C1N(c2ccc(Br)cc2)C(O)C(c2ccccc2)(C2CC2)N1c1ccc(Br)cc1 ; O=C1N(c2ccc(Br)cc2)C(O)[C@@](c2ccccc2)(C2CC2)N1c1ccc(Br)cc1]

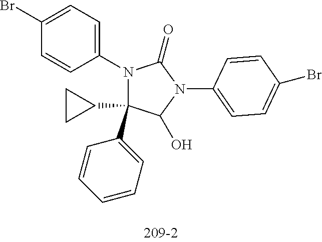 [SMILES: O=C1N(c2ccc(Br)cc2)C(O)[C@](c2ccccc2)(C2CC2)N1c1ccc(Br)cc1]

To a solution of Compound 1 (20 mg, 0.041 mmol) in dichloromethane (5 mL) was added TFA (3 drops). The mixture was stirred at room temperature for 2 hours. The reaction mixture was concentrated and the residue was purified by preparative HPLC to give Compound 209A. LC-MS (ESI) m/z: 469 [M+H]⁺; ¹H-NMR (DMSO-d₆, 400 MHz): δ (ppm) 7.15-7.17 (m, 2H), 7.20-7.22 (m, 2H), 7.30-7.32 (m, 3H), 7.61-7.64 (m, 3H), 7.68-7.71 (m, 2H), 7.82-7.84 (m, 2H).

Compound 209B was synthesized by employing the procedure described for Compound 86B using Compound 209A in lieu of Compound 86A. LC-MS (ESI) m/z: 991 [2M+Na]⁺; ¹H-NMR (CDCl₃, 400 MHz): δ (ppm) 5.21-5.48 (m, 1H), 7.18-7.33 (m, 9H), 7.40-7.81 (m, 4H).

Compound 209 was synthesized by employing the procedure described for Compound 86 using Compound 209B and cyclopropylmagnesium bromide in lieu of Compound 86B and methylmagnesium bromide, which was separated with chiral HPLC to yield Compound 209-1 and Compound 209-2. Compound 209-1: LC-MS (ESI) m/z: 527 [M+H]⁺; ¹H-NMR (CDCl₃, 400 MHz): δ (ppm) 0.08-0.50 (m, 5H), 4.96, 5.36 (s, 1H), 7.19-7.22 (m, 1H), 7.29-7.42 (m, 10H), 7.47-7.51 (m, 2H). Chiral separation condition: MeOH contained 0.2% Methanol Ammonia; OD-H (4.6*150 mm, 5 μm); retention time: 4.7 minutes, (80%), 9.8 minutes (20%). Compound 209-2: LC-MS (ESI) m/z: 527 [M+H]⁺; ¹H-NMR (CDCl₃, 400 MHz): δ (ppm) 0.08-0.50 (m, 5H), 4.96, 5.36 (m, 1H), 7.19-7.22 (m, 1H), 7.29-7.42 (m, 10H), 7.47-7.51 (m, 2H). Chiral separation condition: MeOH contained 0.2% Methanol Ammonia; OD-H (4.6*150 mm, 5 μm); retention time: 2.45 minutes (20%), 3.38 minutes (80%).

### Example 210

### Synthesis of 1,3-bis(4-bromophenyl)-5-hydroxy-5-(1H-indol-2-yl)imidazolidine-2,4-dione

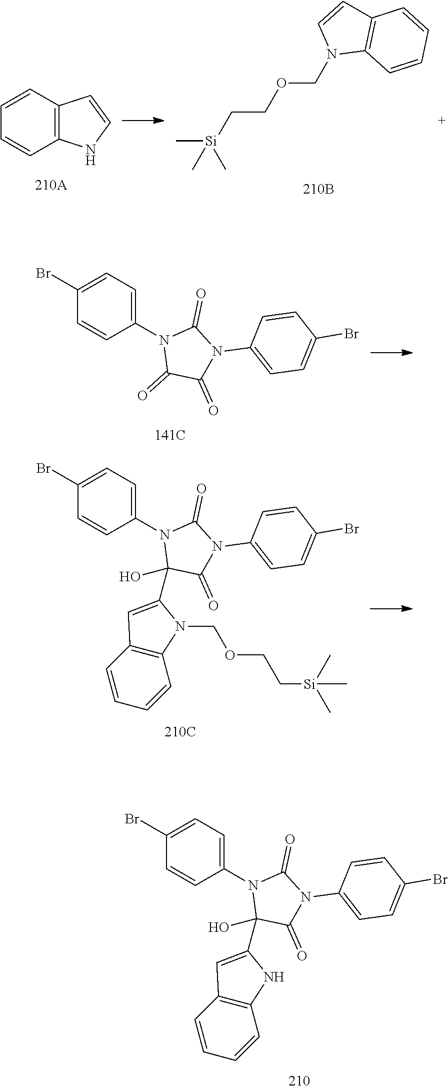 [SMILES: c1ccc2[nH]ccc2c1 ; C[Si](C)(C)CCOCn1ccc2ccccc21 ; O=C1C(=O)N(c2ccc(Br)cc2)C(=O)N1c1ccc(Br)cc1 ; C[Si](C)(C)CCOCn1c(C2(O)C(=O)N(c3ccc(Br)cc3)C(=O)N2c2ccc(Br)cc2)cc2ccccc21 ; O=C1N(c2ccc(Br)cc2)C(=O)C(O)(c2cc3ccccc3[nH]2)N1c1ccc(Br)cc1]

To a solution of Compound 210A (500 mg, 4.3 mmol) in DMF (10 mL) was added NaH (205 mg, 5.1 mmol) at 0° C. After the mixture was stirred at 0° C. for 20 minutes, to it was added (2-(chloromethoxy)ethyl)trimethylsilane (851 mg, 5.1 mmol) and stirred at 0° C. for 1 hour. The mixture was poured into water (50 mL) and extracted with ethyl acetate (50 mL×2). The combined organic layers was washed with water (50 mL×3) and brine (50 mL), dried over Na₂SO₄, filtered, and concentrated to give a crude product, which was purified with flash column chromatography on silica gel (ethyl acetate in petroleum ether, 10% v/v) to afford Compound 210B. LC-MS (ESI) m/z: 248 [M+H]⁺; ¹H-NMR (CDCl₃, 400 MHz): δ (ppm) −0.001 (s, 9H), 0.945 (t, J=8.0 Hz, 2H), 3.53 (t, J=8.0 Hz, 2H), 5.55 (s, 2H), 6.58-6.59 (m, 1H), 7.18-7.23 (m, 2H), 7.28-7.32 (m, 1H), 7.56 (d, J=8.0 Hz, 1H), 7.69 (d, J=8.0 Hz, 1H).

Compound 210C was synthesized by employing the procedure described for Compound 127 using Compound 141C and 210B lieu of Compound 2C and 1-bromo-4-(tert-butyl)benzene. LC-MS (ESI) m/z: 692 [M+Na]⁺. ¹H-NMR (CDCl₃, 400 MHz): δ (ppm) −0.001 (s, 9H), 0.81-0.86 (m, 1H), 0.99-1.06 (m, 1H), 3.53-3.70 (m, 2H), 5.63 (d, J=11.6 Hz, 1H), 6.33-6.36 (m, 1H), 6.76 (s, 1H), 7.23 (d, J=7.6 Hz, 1H), 7.26-7.49 (m, 4H), 7.53-7.55 (m, 2H), 7.62-7.68 (m, 5H).

To a solution of Compound 210C (50 mg, 0.07 mmol) in THF (5 mL) was added 2 NHCl (5 mL). The mixture was heated at reflux for 12 hours. After cooled down to room temperature, the mixture was poured into water (50 mL) and extracted with ethyl acetate (50 mL×2). The combined organic layers was washed with saturated NaHCO₃ solution (50 mL) and brine (50 mL), dried over Na₂SO₄, filtered, and concentrated to give a crude product, which was purified with preparative HPLC to furnished Compound 210. LC-MS (ESI) m/z: 540 [M+H]⁺; ¹H-NMR (CDCl₃, 400 MHz): δ (ppm) 6.44 (s, 1H), 7.13 (t, J=7.2 Hz, 1H), 7.22-7.24 (m, 1H), 7.27-7.30 (m, 2H), 7.34 (d, J=8.0 Hz, 1H), 7.41-7.48 (m, 4H), 7.54-7.58 (m, 3H), 8.71 (s, 1H).

### Example 211

### Synthesis of 1,3-bis(4-bromophenyl)-5-hydroxy-5-(1H-pyrrol-2-yl)imidazolidine-2,4-dione

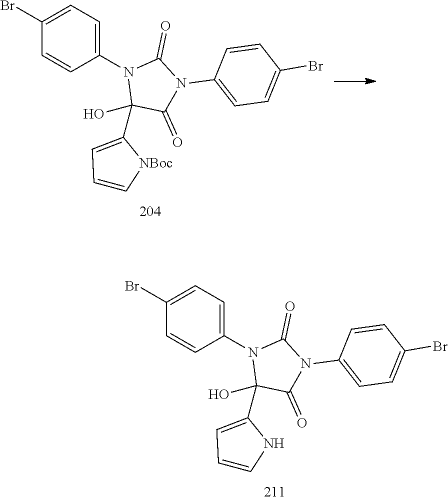 [SMILES: CC(C)(C)OC(=O)n1cccc1C1(O)C(=O)N(c2ccc(Br)cc2)C(=O)N1c1ccc(Br)cc1 ; O=C1N(c2ccc(Br)cc2)C(=O)C(O)(c2ccc[nH]2)N1c1ccc(Br)cc1]

To a solution of Compound 204 (20 mg, 0.034 mmol) in dichloromethane (20 mL) was dropped 2,2,2-trifluoroacetic acid (4 mL) at 0° C. over 5 minutes. The mixture was stirred at the room temperature for 3 hours, poured into saturated aqueous sodium bicarbonate solution (50 mL), and extracted with ethyl acetate (50 mL×3). The combined organic layers was washed with brine (100 mL), dried over anhydrous sodium sulfate, filtered, and concentrated to give a crude product, which was purified with preparative HPLC to furnish Compound 211. LC-MS (ESI) m/z: 492 [M+H]⁺; ¹H-NMR (CDCl₃, 400 MHz): δ (ppm) 4.95 (brs, 1H), 6.06-6.07 (m, 1H), 6.14-6.15 (m, 1H), 6.76-6.77 (m, 1H), 7.24-7.26 (m, 2H), 7.39-7.47 (m, 4H), 7.55-7.57 (m, 2H), 8.87-8.89 (brs, 1H).

### Example 212

### Synthesis of 3-(8-hydroxy-6-oxo-5,7-bis(4-(trifluoromethyl)phenyl)-5,7-diazaspiro[3.4]octan-8-yl)benzonitrile

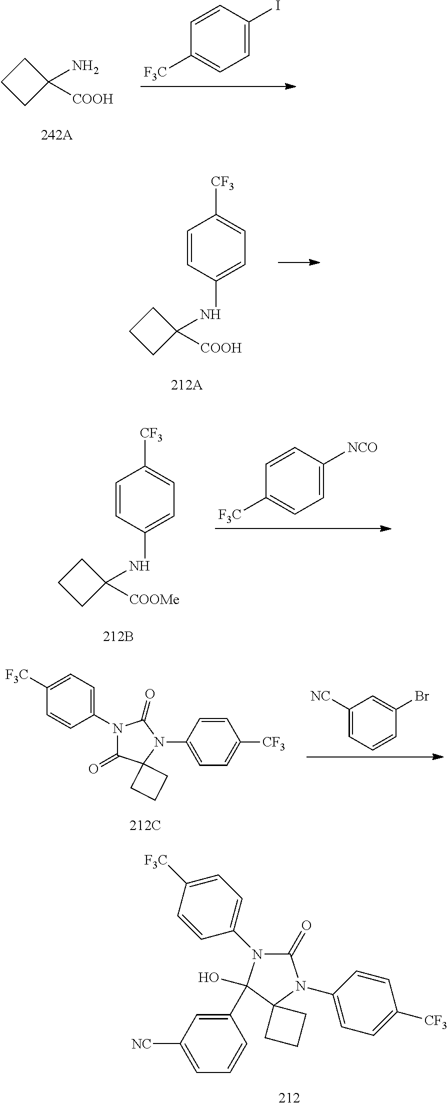

Compounds 212A, 212B, 212C, and 212 were synthesized by employing the procedures described for Compounds 224B, 224C, 224D, and 125 using 1-iodo-4-(trifluoromethyl)benzene, Compounds 242A, 212A, 212B, 1-isocyanato-4-(trifluoromethyl)benzene, 212C, and 3-bromobenzonitrile in lieu of methyl 1-aminocyclopropane-1-carboxylate, Compounds 224A, 224B, 224C, 1-chloro-4-isocyanatobenzene, 2C, and 3-bromo-N,N-dimethylaniline. Compound 212A. LC-MS (ESI) m/z: 258 [M−H]. Compound 212B. LC-MS (ESI) m/z: 274 [M+H]⁺; ¹H-NMR (400 MHz, CDCl₃): δ (ppm) 2.07-2.17 (m, 2H), 2.19-2.24 (m, 2H), 2.72-2.78 (m, 2H), 3.70 (s, 3H), 4.56 (s, 1H), 6.44 (d, J=8.4 Hz, 2H), 7.38 (d, J=8.4 Hz, 2H). Compound 212C. LC-MS (ESI) m/z: 429 [M+H]⁺. Compound 212. LC-MS (ESI) m/z: 532 [M+H]⁺. ¹H-NMR (400 MHz, CDCl₃): δ (ppm) 0.82-0.90 (m, 1H), 1.17-1.25 (m, 1H), 1.75-1.82 (m, 1H), 2.07-2.12 (m, 1H), 2.36-2.52 (m, 1H), 2.65-2.80 (m, 1H), 7.45-7.57 (m, 6H), 7.66-7.82 (m, 6H).

### Example 213

### Synthesis of 5-(benzofuran-2-yl)-1,3-bis(4-bromophenyl)-5-hydroxyimidazolidine-2,4-dione

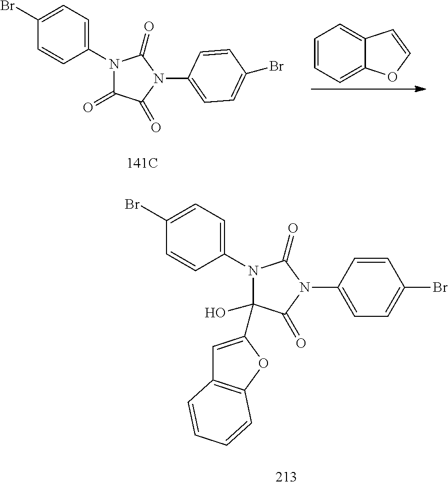

Compound 213 was synthesized by employing the procedure described for Compound 127 using Compound 141C and benzofuran lieu of Compound 2C and 1-bromo-4-(tert-butyl)benzene. LC-MS (ESI) m/z: non-ionizable compound under routine conditions used; ¹H-NMR (DMSO-d₆, 400 MHz): δ (ppm) 7.18 (s, 1H), 7.27 (t, J=7.2 Hz, 1H), 7.31-7.42 (m, 3H), 7.49 (d, J=8.7 Hz, 2H), 7.53-7.69 (m, 4H), 7.80 (d, J=8.7 Hz, 2H), 8.73 (s, 1H).

### Example 214

### Synthesis of 5-(benzo[b]thiophen-2-yl)-1,3-bis(4-bromophenyl)-5-hydroxyimidazolidine-2,4-dione

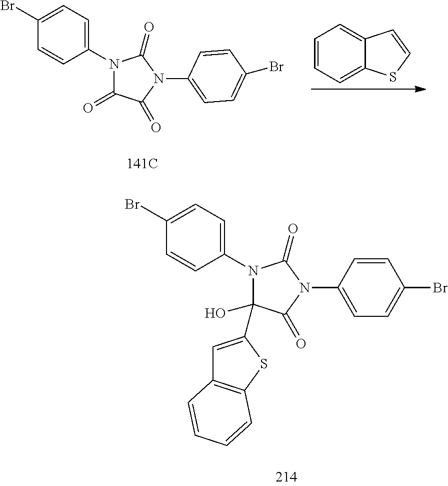

Compound 214 was synthesized by employing the procedure described for Compound 127 using Compound 141C and benzo[b]thiophene lieu of Compound 2C and 1-bromo-4-(tert-butyl)benzene. LC-MS (ESI) m/z: 557 [M+H]⁺; ¹H-NMR (CDCl₃, 400 MHz): δ (ppm) 5.00 (s, 1H), 7.32-7.47 (m, 9H), 7.58 (d, J=8.8 Hz, 2H), 7.71-7.79 (m, 2H).

### Example 215

### Synthesis of 1,3-bis(4-bromophenyl)-5-(2,3-dihydro-1H-inden-2-yl)-5-hydroxyimidazolidine-2,4-dione

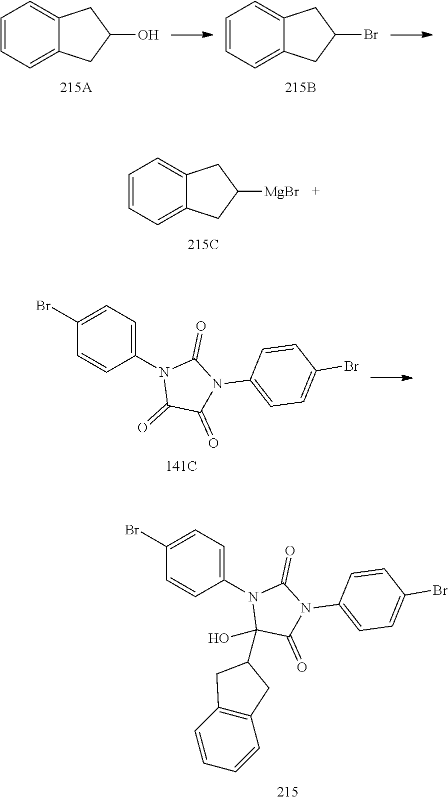

To a solution of 2,3-dihydro-1H-inden-2-ol 215A (1.6 g, 12 mmol) in CHCl₃ (40 mL) was added pyridine (0.24 mL) and PBr₃ (1.28 mL) at 0° C. The mixture was heated at reflux for 1 hour and stirred at room temperature overnight. The reaction mixture was poured into ice-water (50 mL) and extracted with dichloromethane (30 mL×3). The combined organic layers was washed with brine (40 mL), dried over anhydrous sodium sulfate, filtered, and concentrated under reduced pressure. The residue was purified with column chromatography on silica gel (petroleum ether, 100% v/v) to furnish Compound 215B. LC-MS (ESI) m/z: non-ionizable compound under routine conditions used; ¹H-NMR (CDCl₃, 400 MHz): δ (ppm) 3.32-3.37 (m, 2H), 3.49-3.55 (m, 2H), 4.74-4.79 (m, 1H), 7.20-7.28 (m, 4H).

Compounds 215C and 215 were synthesized by employing the procedures described for Compounds 174B and 141 using Compounds 215B and 215C in lieu of Compound 174A and phenylmagnesium bromide.

Compound 215C as a Grignard reagent, which was cooled down to room temperature and used directly in the next step. Compound 215. LC-MS (ESI) m/z: 541 [M+H]⁺; ¹H-NMR (DMSO-d₆, 400 MHz): δ (ppm) 2.67-2.74 (m, 1H), 2.87-2.91 (m, 3H), 3.44-3.46 (m, 1H), 7.08-7.16 (m, 4H), 7.36-7.38 (m, 2H), 7.50-7.52 (m, 2H), 7.66-7.68 (m, 2H), 7.72-7.74 (m, 2H), 7.87 (s, 1H).

### Example 216

### Synthesis of 1,3-bis(4-bromophenyl)-5-hydroxy-5-(trifluoromethyl)imidazolidine-2,4-dione

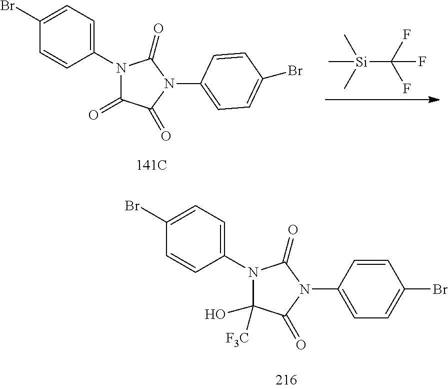

To a solution of Compound 141C (212 mg, 0.5 mmol) in THF (10 mL) was added TMSCF₃ (0.074 mL, 0.5 mmol) at 0° C. under nitrogen. To the solution was dropped a solution tetrabutylammonium fluoride in THF (1M, 0.05 mL, 0.05 mmol). After stirred at 20° C. for 6 hours, the reaction mixture was quenched with ethyl acetate (20 mL×3), washed with brine (40 mL), dried over anhydrous sodium sulfate, filtered, and concentrated under reduced pressure. The residue was purified with preparative HPLC to give Compound 216. LC-MS: (ESI) m/z: 493 [M+H]⁺; ¹H-NMR (Acetone-d₆, 400 MHz): δ (ppm) 4.71 (s, 1H), 7.35 (t, J=8 Hz, 4H), 7.57-7.64 (m, 4H).

### Example 217

### Synthesis of ethyl 1,3-bis(4-chlorophenyl)-4-hydroxy-2-oxo-5-phenylimidazolidine-4-carboxylate

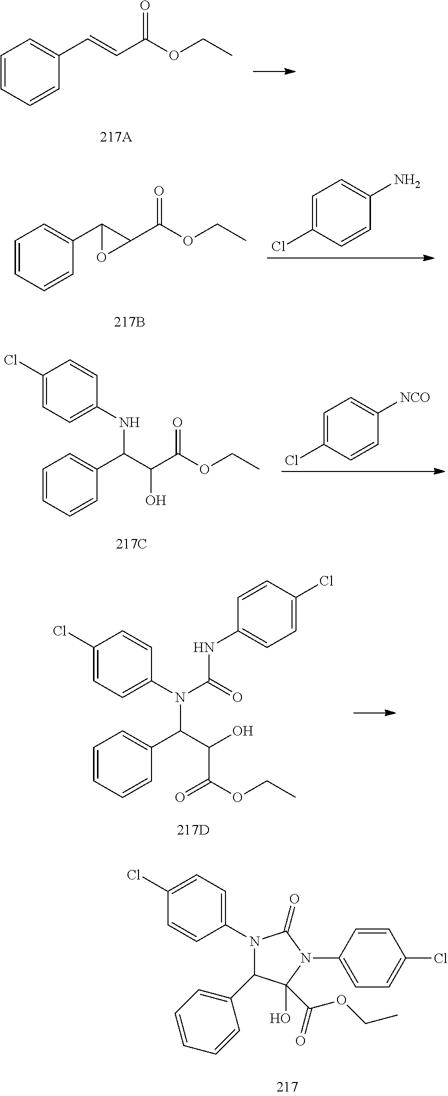

Compounds 217B, 217C, 217D, and 217 were synthesized by employing the procedures described for Compounds 84B, 122C, 1, and 14 using Compounds 217A, stirred at 50° C., 217B, 217C, 1-chloro-4-isocyanatobenzene, and 217D in lieu of Compounds 84A, stirred at room temperature, 122B, 1B, 1-bromo-4-isocyanatobenzene, and 14D. Compound 217B. LC-MS (ESI) m/z: non-ionizable compound under routine conditions used. ¹H-NMR (CDCl₃, 400 MHz): δ (ppm) 1.33 (t, J=7.2 Hz, 3H), 3.80 (d, J=2 Hz, 1H), 4.08 (d, J=1.6 Hz, 1H), 4.27-4.30 (m, 2H), 7.28-7.36 (m, 2H), 7.35-7.37 (m, 3H). Compound 217C. LC-MS (ESI) m/z: 320 [M+H]⁺; ¹H-NMR (CDCl₃, 400 MHz): δ (ppm) 1.28 (t, J=7.2 Hz, 3H), 2.88 (d, J=7.6 Hz, 1H), 4.15-4.23 (m, 2H), 4.66 (q, J=3.2 Hz, 1H), 4.81 (q, J=3.2 Hz, 1H), 4.94 (q, J=8.4 Hz, 1H), 6.51-6.53 (m, 2H), 7.03-7.05 (m, 2H), 7.25-7.26 (m, 2H), 7.29-7.30 (m, 3H). Compound 217D. LC-MS (ESI) m/z: 473 [M+H]⁺; ¹H-NMR (CDCl₃, 400 MHz): δ (ppm) 1.15 (t, J=7.2 Hz, 3H), 3.20 (d, J=5.6 Hz, 1H), 4.19 (q, J=4 Hz, 2H), 4.89 (q, J=1.6 Hz, 1H), 5.88 (s, 1H), 5.96 (d, J=7.2 Hz, 1H), 7.00 (s, 1H), 7.08-7.15 (m, 2H), 7.16-7.25 (m, 10H). Compound 217. LC-MS (ESI) m/z: 471 [M+H]⁺; ¹H-NMR (CDCl₃, 400 MHz): δ (ppm) 0.85, 1.22 (t, J=7.2 Hz, 3H), 3.38 (s, 1H), 4.25-4.35 (m, 2H), 5.49 (s, 1H), 7.19-7.22 (m, 4H), 7.28-7.42 (m, 9H).

### Example 218

### Synthesis of 1,3-bis(4-chlorophenyl)-4-hydroxy-4-(hydroxymethyl)-5-phenylimidazolidin-2-one, (4S,5R)-1,3-bis(4-chlorophenyl)-4-hydroxy-4-(hydroxymethyl)-5-phenylimidazolidin-2-one, and (4R,5S)-1,3-bis(4-chlorophenyl)-4-hydroxy-4-(hydroxymethyl)-5-phenylimidazolidin-2-one

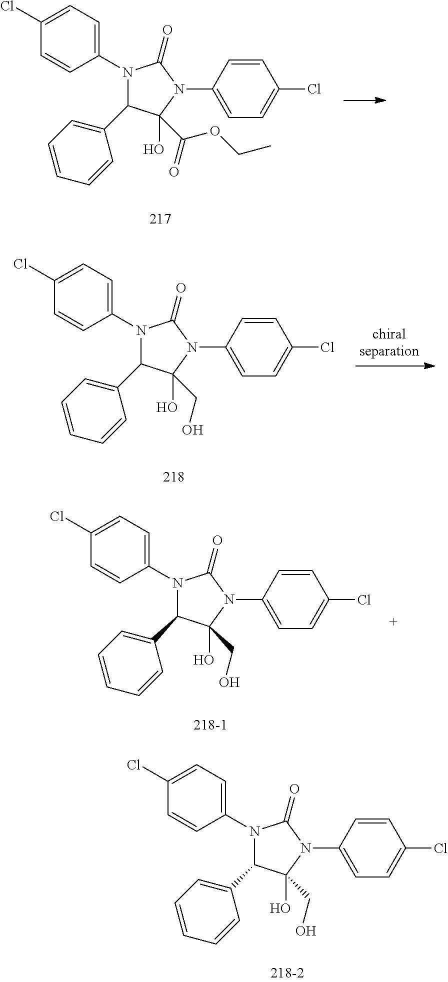 [SMILES: CCOC(=O)C1(O)C(c2ccccc2)N(c2ccc(Cl)cc2)C(=O)N1c1ccc(Cl)cc1 ; O=C1N(c2ccc(Cl)cc2)C(c2ccccc2)C(O)(CO)N1c1ccc(Cl)cc1 ; O=C1N(c2ccc(Cl)cc2)[C@H](c2ccccc2)[C@](O)(CO)N1c1ccc(Cl)cc1 ; O=C1N(c2ccc(Cl)cc2)[C@@H](c2ccccc2)[C@@](O)(CO)N1c1ccc(Cl)cc1]

To a solution of LiAlH₄ (150 mg, 3.95 mmol) in dry THF (10 mL) was dropped a solution of Compound 217 (350 mg, 0.743 mmol) in THF (2 mL) at 0° C. under nitrogen. The mixture was stirred at 0° C. for 5 hours, poured into ice-water (20 mL), and extracted with ethyl acetate (20 mL×2). The combined organic layers was washed with brine (10 mL), dried over anhydrous sodium sulfate, filtered, and concentrated under reduced pressure. The residue was purified with preparative HPLC to furnish Compound 218. LC-MS (ESI) m/z: 429 [M+H]⁺; ¹H-NMR (CDCl₃, 400 MHz): δ (ppm) 1.77 (t, J=6 Hz, 1H), 2.28 (s, 1H), 3.61-3.76 (m, 2H), 5.58 (s, 1H), 7.20 (d, J=8.8 Hz, 2H), 7.36-7.45 (m, 11H).

Compound 218 was separated with chiral HPLC to give Compound 218-1 and Compound 218-2. Compound 218-1: LC-MS (ESI) m/z: 429 [M+H]⁺; ¹H-NMR (CDCl₃, 400 MHz): δ (ppm) 3.63-3.75 (m, 2H), 5.58 (s, 1H), 7.18-7.36 (m, 2H), 7.36-7.45 (m, 11H). Chiral separation condition: MeOH contained 0.2% Methanol Ammonia; AD-H (4.6*100 mm, 5 μm); retention time: 2.19 minutes. Compound 218-2: LC-MS (ESI) m/z: 429 [M+H]⁺; ¹H-NMR (CDCl₃, 400 MHz): δ (ppm) 3.63-3.76 (m, 2H), 5.58 (s, 1H), 7.18-7.21 (m, 2H), 7.36-7.45 (m, 11H). Chiral separation condition: MeOH contained 0.2% Methanol Ammonia; AD-H (4.6*100 mm, 5 μm); retention time: 3.83 minutes.

### Example 219

### Synthesis of 1,3-bis(4-bromophenyl)-5-hydroxy-4-methyl-4-(3-(trifluoromethyl)phenyl)imidazolidin-2-one, (4S)-1,3-bis(4-bromophenyl)-5-hydroxy-4-methyl-4-(3-(trifluoromethyl)phenyl)imidazolidin-2-one, and (4R)-1,3-bis(4-bromophenyl)-5-hydroxy-4-methyl-4-(3-(trifluoromethyl)phenyl)imidazolidin-2-one

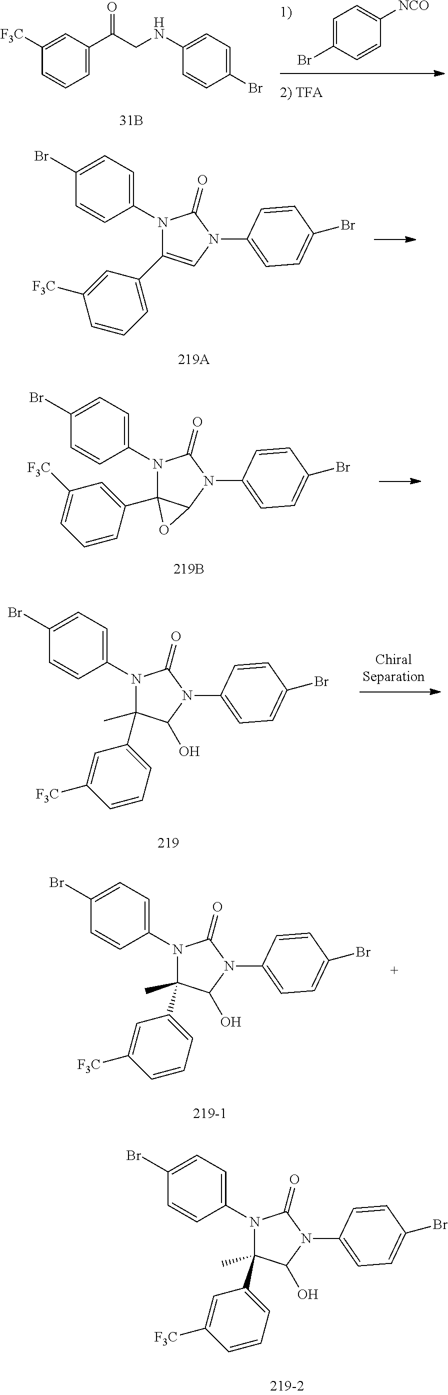

Compounds 219A, 219B, and 219 were synthesized by employing the procedures described for Compounds 86A, 86A, and 86 using Compounds 31B, 219A, and 219B in lieu of Compounds 1B, 86A, and 86B. Compound 238A. LC-MS (ESI) m/z: 537 [M+H]⁺; ¹H-NMR (DMSO-d₆, 400 MHz): δ (ppm) 7.26 (d, J=8.4 Hz, 2H), 7.32 (d, J=8.0 Hz, 1H), 7.53 (t, J=8.0 Hz, 1H), 7.56 (s, 1H), 7.62 (d, J=8.0 Hz, 1H), 7.66 (d, J=8.8 Hz, 2H), 7.71 (d, J=8.8 Hz, 2H), 7.83-7.85 (m, 3H). Compound 219B. LC-MS (ESI) m/z: 553 [M+H]⁺; ¹H-NMR (CDCl₃, 400 MHz): δ (ppm) 5.67 (s, 1H), 7.37 (d, J=8.8 Hz, 2H), 7.40 (d, J=8.8 Hz, 2H), 7.47 (d, J=8.8 Hz, 2H), 7.56-7.57 (m, 2H), 7.63 (d, J=8.8 Hz, 2H), 7.65-7.68 (m, 2H).

Compound 219 was separated with chiral-HPLC to yield Compound 219-1 and Compound 219-2. Compound 219-1: LC-MS (ESI) m/z: 569 [M+H]⁺; ¹H-NMR (CDCl₃, 400 MHz): δ (ppm) 0.87-1.59 (ds, 3H), 5.07-5.17 (ds, 1H), 7.11-7.35 (m, 7H), 7.44-7.63 (m, 5H). Chiral separation condition: Methanol contained 0.2% methanol ammonia; OZ-H (250*4.6 mm, 5 μm); retention time: 1.79 minutes (40%), 2.07 minutes (60%). Compound 219-2: LC-MS (ESI) m/z: 569 [M+H]⁺; ¹H-NMR (CDCl₃, 400 MHz): δ (ppm) 0.89-1.60 (ds, 3H), 5.07-5.18 (ds, 1H), 7.12-7.35 (m, 7H), 7.45-7.62 (m, 5H). Chiral separation condition: Methanol contained 0.2% methanol ammonia; OZ-H (250*4.6 mm, 5 μm); retention time: 2.89 minutes (50%), 3.26 minutes (50%).

### Example 220

### Synthesis of 1,3-bis(4-chlorophenyl)-5-hydroxy-4-isopropyl-4-(3-(trifluoromethoxy)phenyl)imidazolidin-2-one

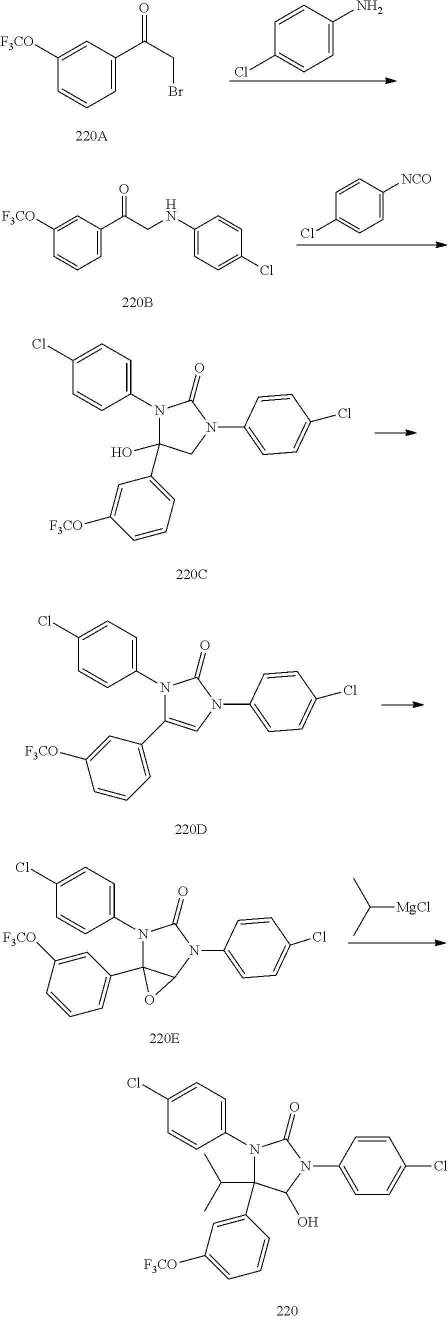

Compounds 220B and 220C were synthesized by employing the procedures described for Compounds 1B and 12 using Compound 220A and 4-chloroaniline, and Compound 220B using NaHCO₃ as base in lieu of Compound 1A and 4-bromoaniline, and Compound 12B using DIPEA as base. Compound 220B. LC-MS (ESI) m/z: 330 [M+H]⁺. Compound 220C. LC-MS: (ESI) m/z: 483 [M+H]⁺.

Compound 220D was synthesized by employing the procedures described for Compounds 209A using Compound 220C in lieu of Compound 1. LC-MS (ESI) m/z: 465 [M+H]⁺; ¹H-NMR (Acetone-d₆, 400 MHz): δ (ppm) 7.14 (s, 1H), 7.24-7.28 (m, 2H), 7.28-7.34 (m, 2H), 7.45-7.56 (m, 6H), 7.94 (d, J=9.2 Hz, 2H).

Compounds 220E and 220 were synthesized by employing the procedures described for Compounds 86B and 86 using Compound 220D, and Compound 220E and isopropylmagnesium chloride in lieu of Compound 86A, and Compound 86B and phenylmagnesium bromide. Compound 220E. LC-MS (ESI) m/z: 481 [M+H]⁺; ¹H-NMR (CDCl₃, 400 MHz): δ (ppm) 5.61 (s, 1H), 7.24-7.33 (m, 6H), 7.39-7.47 (m, 6H). Compound 220. LC-MS (ESI) m/z: 525 [M+H]⁺; ¹H-NMR (Acetone-d₆, 400 MHz): δ (ppm) 1.05 (d, J=6.8 Hz, 3H), 1.18 (d, J=6.8 Hz, 3H), 2.19-2.25 (m, 1H), 5.71 (s, 1H), 7.23-7.25 (m, 3H), 7.42-7.50 (m, 5H), 7.60-7.64 (m, 4H).

### Example 221

### Synthesis of 3-(1,3-bis(4-chlorophenyl)-4-hydroxy-2-oxo-7-oxa-1,3-diazaspiro[4.4]nonan-4-yl)benzonitrile, 3-((5S)-1,3-bis(4-chlorophenyl)-4-hydroxy-2-oxo-7-oxa-1,3-diazaspiro[4.4]nonan-4-yl)benzonitrile, and 3-((5R)-1,3-bis(4-chlorophenyl)-4-hydroxy-2-oxo-7-oxa-1,3-diazaspiro[4.4]nonan-4-yl)benzonitrile

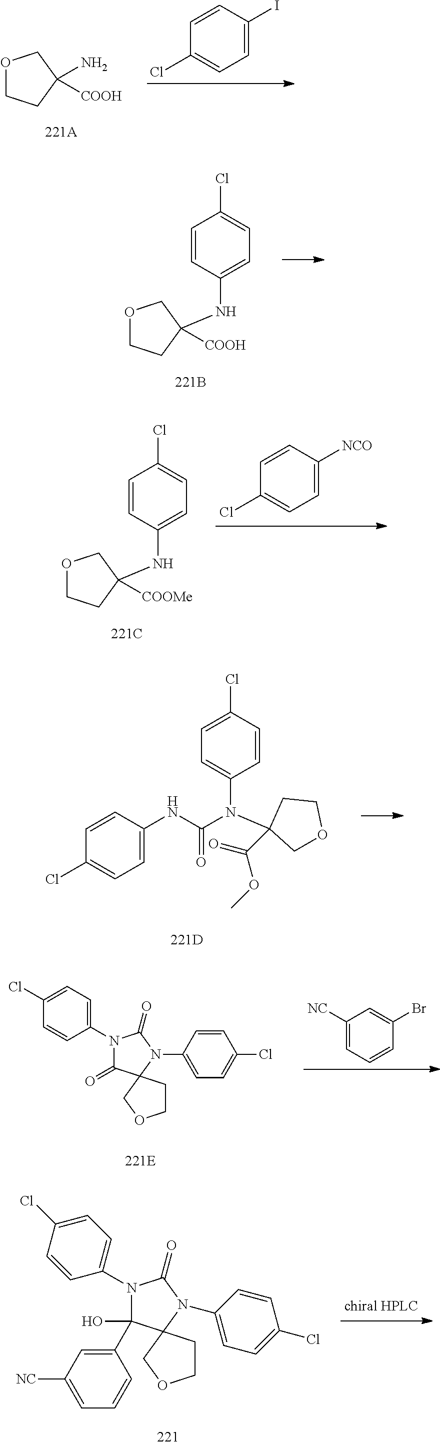

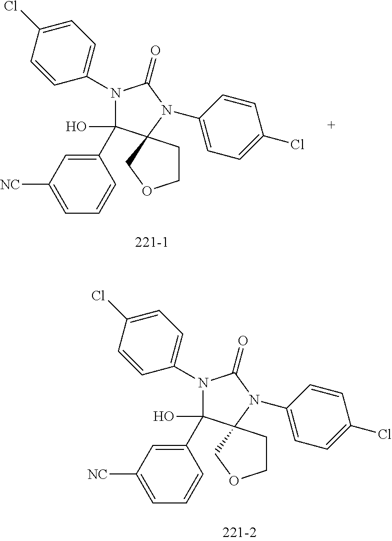 [SMILES: N#Cc1cccc(C2(O)N(c3ccc(Cl)cc3)C(=O)N(c3ccc(Cl)cc3)[C@]23CCOC3)c1 ; N#Cc1cccc(C2(O)N(c3ccc(Cl)cc3)C(=O)N(c3ccc(Cl)cc3)[C@@]23CCOC3)c1]

Compounds 221B, 221C, 221D, 221E, and 221 were synthesized by employing the procedures described for Compounds 224B, 224C, 224D, 224E, and 125 using Compounds 221A, 221B, 221C, 221D, 221E, and 3-bromobenzonitrile in lieu of Compounds 224A, 224B, 224C, 224D, 2C, and 3-bromo-N,N-dimethylaniline. Compound 221B. LC-MS (ESI) m/z: 240 [M−H]. Compound 221C. LC-MS (ESI) m/z: 256 [M+H]⁺. Compound 221D. LC-MS (ESI) m/z: 409 [M+H]⁺. Compound 221E. LC-MS (ESI) m/z: 377 [M+H]⁺. Compound 221. LC-MS (ESI) m/z: 480 [M+H]⁺. ¹H-NMR (400 MHz, CDCl₃): δ (ppm) 1.59-1.64, 2.13-2.18 (m, 1H), 1.86-1.91, 2.57-2.60 (m, 1H), 2.93-2.98, 3.50-3.54 (m, 1H), 3.18-3.21, 4.09-4.13 (m, 1H), 3.21-3.25, 3.65-3.67 (d, J=8.4 Hz, 1H), 4.03-4.05, 4.19-4.21 (d, J=8.4 Hz, 1H), 4.96, 5.21 (s, 1H), 7.14-7.25 (m, 6H), 7.29-7.32 (m, 2H), 7.38-7.43 (m, 1H), 7.51-7.61 (m, 2H), 7.78-7.81 (m, 1H).

Compound 221 was separated by using chiral HPLC to give Compound 221-1 and Compound 221-2. Compound 221-1: LC-MS (ESI) m/z: 480 [M+H]⁺. ¹H-NMR (400 MHz, CDCl₃): δ (ppm) 1.64-1.70, 2.16-2.21 (m, 1H), 1.92-1.95, 2.60-2.65 (m, 1H), 2.98-2.99, 3.50-3.54 (m, 1H), 3.18-3.21, 4.09-4.13 (m, 1H), 3.21-3.25, 4.04 (d, J=8.4 Hz, 1H), 3.66, 4.20 (d, J=8.4 Hz, 1H), 4.32, 4.61 (s, 1H), 7.14-7.25 (m, 6H), 7.29-7.32 (m, 2H), 7.38-7.43 (m, 1H), 7.51-7.61 (m, 2H), 7.78-7.81 (m, 1H). Chiral separation conditions: MeOH contained 0.2% NH₄OH; cellulose-SC (100×4.6 mm, 5 μm); retention time: 1.44 minutes (56%), 2.77 minutes (44%). Compound 221-2: LC-MS (ESI) m/z: 480 [M+H]⁺. ¹H-NMR (400 MHz, CDCl₃): δ (ppm) 1.63-1.68, 2.14-2.21 (m, 1H), 1.89-1.95, 2.58-2.66 (m, 1H), 2.98-2.99, 3.52-3.56 (m, 1H), 3.18-3.21, 4.06-4.08 (m, 1H), 3.22, 4.04 (d, J=8.4 Hz, 1H), 3.66, 4.20 (d, J=8.4 Hz, 1H), 4.60, 4.78 (s, 1H), 7.14-7.25 (m, 6H), 7.29-7.32 (m, 2H), 7.38-7.43 (m, 1H), 7.51-7.61 (m, 2H), 7.78-7.81 (m, 1H). Chiral separation conditions: MeOH contained 0.2% NH₄OH; cellulose-SC (100×4.6 mm, 5 μm); retention time: 1.97 minutes (67%), 4.41 minutes (33%).

### Example 222

### Synthesis of 1,3-bis(4-chlorophenyl)-5-hydroxy-4-methyl-4-(m-tolyl)imidazolidin-2-one, (4S)-1,3-bis(4-chlorophenyl)-5-hydroxy-4-methyl-4-(m-tolyl)imidazolidin-2-one, and (4R)-1,3-bis(4-chlorophenyl)-5-hydroxy-4-methyl-4-(m-tolyl)imidazolidin-2-one

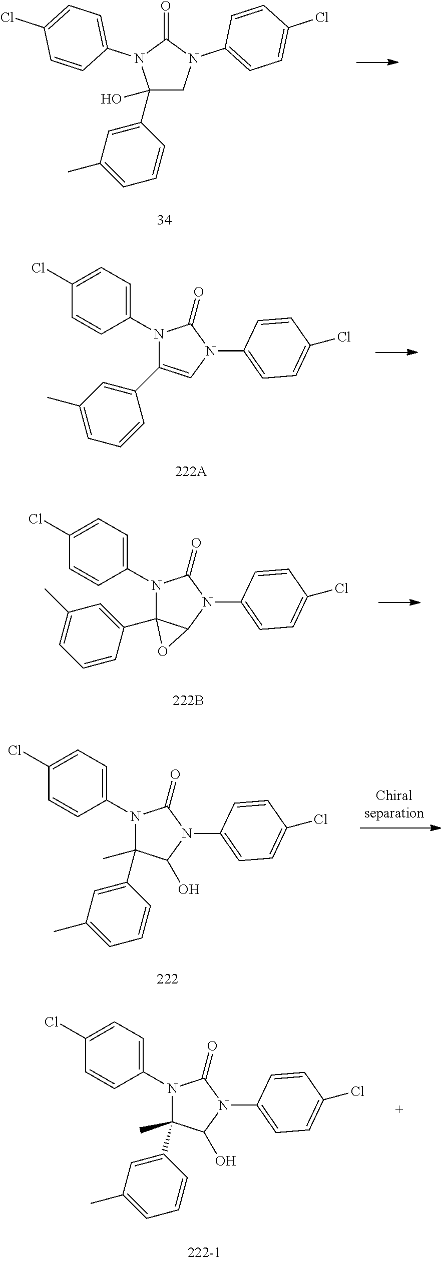 [SMILES: Cc1cccc(C2(O)CN(c3ccc(Cl)cc3)C(=O)N2c2ccc(Cl)cc2)c1 ; Cc1cccc(-c2cn(-c3ccc(Cl)cc3)c(=O)n2-c2ccc(Cl)cc2)c1 ; Cc1cccc(C23OC2N(c2ccc(Cl)cc2)C(=O)N3c2ccc(Cl)cc2)c1 ; Cc1cccc(C2(C)C(O)N(c3ccc(Cl)cc3)C(=O)N2c2ccc(Cl)cc2)c1 ; Cc1cccc([C@@]2(C)C(O)N(c3ccc(Cl)cc3)C(=O)N2c2ccc(Cl)cc2)c1]

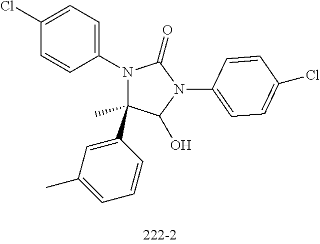 [SMILES: Cc1cccc([C@]2(C)C(O)N(c3ccc(Cl)cc3)C(=O)N2c2ccc(Cl)cc2)c1]

Compound 222A was synthesized by employing the procedure described for Compound 209A using Compound 34 lieu of Compound 1. LC-MS (ESI) m/z: 395 [M+H]⁺; ¹H-NMR (CDCl₃, 400 MHz): δ (ppm) 2.29 (s, 3H), 6.76 (s, 1H), 6.82 (d, J=7.2 Hz, 1H), 6.99 (s, 1H), 7.08-7.20 (m, 4H), 7.33 (d, J=8.0 Hz, 2H), 7.42 (d, J=8.8 Hz, 2H), 7.66 (d, J=8.8 Hz, 2H).

Compounds 222B and 222 were synthesized by employing the procedures described for Compound 86B and 86 using Compound 222A, and Compound 222B and MeMgBr in lieu of Compound 86A, and Compound 86B and phenylmagnesium bromide. Compound 222B. LC-MS (ESI) m/z: 411 [M+H]⁺; ¹H-NMR (CDCl₃, 400 MHz): δ (ppm) 2.35 (s, 3H), 5.52 (s, 1H), 7.06-7.19 (m, 3H), 7.26-7.30 (m, 4H), 7.41-7.49 (m, 5H). Compound 222. LC-MS (ESI) m/z: 427 [M+H]⁺; ¹H-NMR (CDCl₃, 400 MHz): δ (ppm) 0.99 (s, 3H), 2.34 (s, 3H), 5.04 (s, 1H), 6.84-7.19 (m, 6H), 7.33-7.39 (m, 4H), 7.45-7.48 (m, 2H).

Compound 222 was separated with chiral HPLC to yield Compound 222-1 and Compound 222-2. Compound 222-1: LC-MS (ESI) m/z: 427 [M+H]⁺; ¹H-NMR (CDCl₃, 400 MHz): δ (ppm) 0.97 (s, 3H), 2.34 (s, 3H), 5.02 (s, 1H), 6.98-7.24 (m, 6H), 7.31-7.36 (m, 4H), 7.42-7.45 (m, 2H). Chiral separation condition: Methanol contained 0.2% methanol ammonia; AS-H (250*4.6 mm, 5 μm); retention time: 3.6 minute. Compound 222-2: LC-MS (ESI) m/z: 427 [M+H]⁺; ¹H-NMR (CDCl₃, 400 MHz): δ (ppm) 0.97 (s, 3H), 2.33 (s, 3H), 5.02 (s, 1H), 6.97-7.24 (m, 6H), 7.31-7.36 (m, 4H), 7.43-7.45 (m, 2H). Chiral separation condition: Methanol contained 0.2% methanol ammonia; AS-H (250*4.6 mm, 5 μm); retention time: 3.98 minute.

### Example 223

### Synthesis of 1,3-bis(4-chlorophenyl)-4-hydroxy-5-methyl-4-phenylimidazolidin-2-one, (4S)-1,3-bis(4-chlorophenyl)-5-hydroxy-4-methyl-4-phenylimidazolidin-2-one, and (4R)-1,3-bis(4-chlorophenyl)-5-hydroxy-4-methyl-4-phenylimidazolidin-2-one

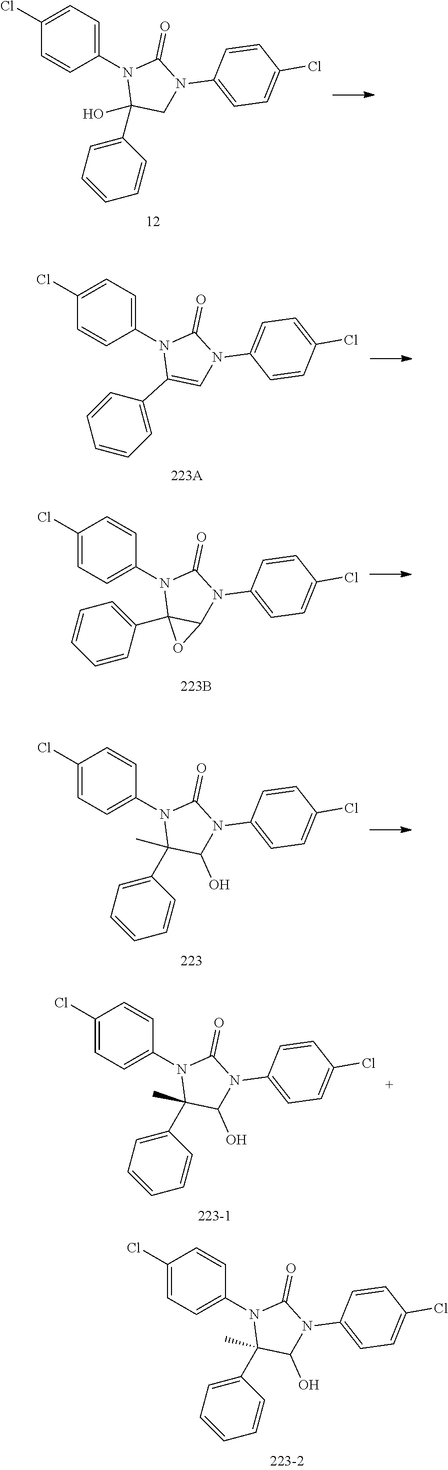 [SMILES: O=C1N(c2ccc(Cl)cc2)CC(O)(c2ccccc2)N1c1ccc(Cl)cc1 ; O=c1n(-c2ccc(Cl)cc2)cc(-c2ccccc2)n1-c1ccc(Cl)cc1 ; O=C1N(c2ccc(Cl)cc2)C2OC2(c2ccccc2)N1c1ccc(Cl)cc1 ; CC1(c2ccccc2)C(O)N(c2ccc(Cl)cc2)C(=O)N1c1ccc(Cl)cc1 ; C[C@]1(c2ccccc2)C(O)N(c2ccc(Cl)cc2)C(=O)N1c1ccc(Cl)cc1 ; C[C@@]1(c2ccccc2)C(O)N(c2ccc(Cl)cc2)C(=O)N1c1ccc(Cl)cc1]

Compounds 223A, 223B, and 223 were synthesized by employing the procedures described for Compounds 209A, 86B, and 86 using Compounds 12, 223A, and 223B in lieu of Compounds 1, 86A, and 86B. Compound 223A. LC-MS (ESI) m/z: 381 [M+H]⁺. Compound 223B. LC-MS (ESI) m/z: 397 [M+H]⁺. Compound 223. LC-MS (ESI) m/z: 413 [M+H]⁺. ¹H-NMR (Acetone-d₆, 400 MHz): δ (ppm) 0.99 (s, 3H), 5.34 (s, 1H), 6.17 (s, 1H), 7.20-7.25 (m, 3H), 7.33-7.35 (m, 2H), 7.40-7.46 (m, 3H), 7.53-7.57 (m, 3H), 7.65-7.68 (m, 2H).

Compound 223 was separated with chiral HPLC to yield Compound 223-1 and Compound 223-2. Compound 223-1: LC-MS (ESI) m/z: 413 [M+H]⁺; ¹H-NMR (CDCl₃, 400 MHz): δ (ppm) 0.97 (s, 3H), 5.07 (s, 1H), 7.13-7.19 (m, 3H), 7.33-7.44 (m, 10H). Chiral separation condition: MeOH contained 0.2% Methanol Ammonia; OJ-H (250*4.6 mm, 5 μm); retention time: 3.4 minutes. Compound 223-2: LC-MS (ESI) m/z: 413 [M+H]⁺; ¹H-NMR (CDCl₃, 400 MHz): δ (ppm) 0.97 (s, 3H), 5.07 (s, 1H), 7.14-7.16 (m, 3H), 7.33-7.44 (m, 10H). Chiral separation condition: MeOH contained 0.2% Methanol Ammonia; OJ-H (250*4.6 mm, 5 μm); retention time: 4.54 minutes.

### Example 224

### Synthesis of 4,6-bis(4-chlorophenyl)-7-hydroxy-7-(3-(trifluoromethoxy)phenyl)-4,6-diazaspiro[2.4]heptan-5-one

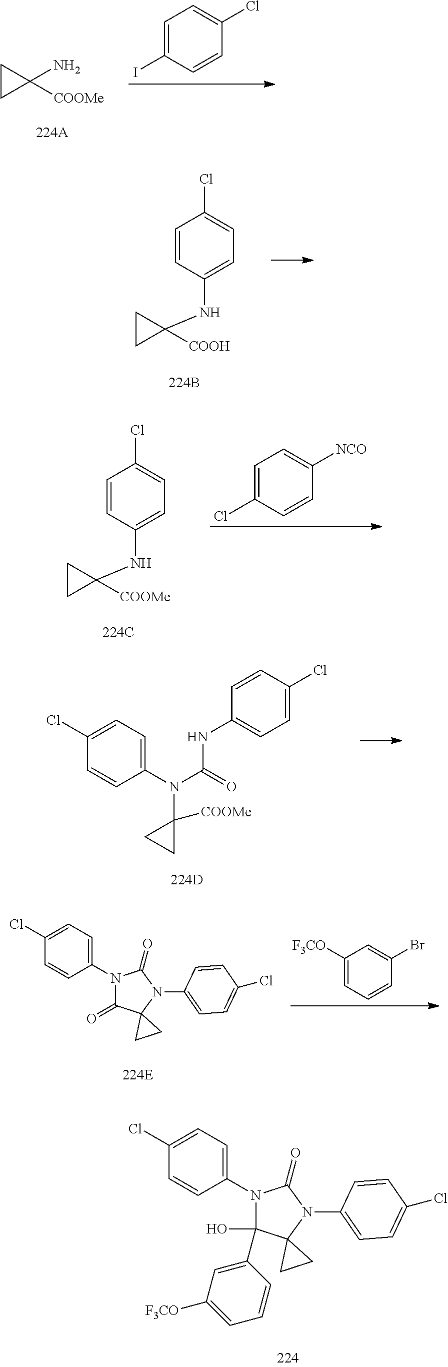

To a solution of Compound 224A (500 mg, 3.29 mmol) and 1-chloro-4-iodobenzene (1.18 g, 4.94 mmol) in DMF/H₂O (6 mL, v/v=5/1) was added K₂CO₃ (1.36 g, 9.84 mmol), Et₃N (33 mg, 0.329 mmol), and CuI (125 mg, 0.656 mmol). The mixture was heated at 110° C. overnight. After cooled to room temperature, the mixture was diluted with H₂O (50 mL) and its pH was adjusted to about 4.0 with aqueous HCl solution (1.0 N). It was extracted with EtOAc (20 mL×2). The combined organic layers was washed with brine (50 mL), dried over anhydrous sodium sulfate, filtered, and concentrated to give a crude Compound 224B, which was used directly in the next step without further purification. LC-MS (ESI) m/z: 212 [M+H]⁺.

To the solution of Compound 224B (694 mg, 3.29 mmol) in methanol (15 ml) was dropped concentrated sulfuric acid (0.5 mL) at room temperature. The mixture was stirred at 60° C. overnight and concentrated. The residue was dissolved in ethyl acetate (15 mL) and washed successively with water (30 mL), 0.5 M aqueous sodium bicarbonate solution (30 mL), and brine (30 mL). The organic layer was dried over anhydrous sodium sulfate, filtered, and concentrated to give a crude product, which was purified with silica gel chromatography using eluents (ethyl acetate in petroleum ether, 20% v/v) to furnish Compound 224C. LC-MS (ESI) m/z: 226 [M+H]⁺.

The mixture of Compound 224C (300 mg, 1.32 mmol) and 1-chloro-4-isocyanatobenzene (216 mg, 1.38 mmol) in toluene (30 mL) was heated at 80° C. overnight. The reaction mixture was concentrated under vacuum and the residue was purified with silica gel chromatography using eluents (ethyl acetate in petroleum ether, 20% v/v) to furnish Compound 224D. LC-MS (ESI) m/z: 379 [M+H]⁺.

To a solution of Compound 224D (232 mg, 0.61 mmol) in MeOH (15 mL) was added a solution of ammonia in methanol (25%, 5 drops). The mixture was stirred at room temperature for 1.5 hours and evaporated to dryness. The resulting solid was recrystallized from EtOAc/petroleum ether to furnish Compound 224E. LC-MS (ESI) m/z: 347 [M+H]⁺. ¹H-NMR (400 MHz, CDCl₃): δ (ppm) 1.19 (m, 2H), 1.57 (m, 2H), 7.19-7.21 (m, 2H), 7.44-7.51 (m, 6H).

Compound 224 was synthesized by employing the procedure described for Compound 127 using Compound 224E and 1-bromo-3-(trifluoromethoxy)benzene in lieu of Compound 2C and 1-bromo-4-(tert-butyl)benzene. LC-MS (ESI) m/z: 509 [M+H]⁺. ¹H-NMR (400 MHz, CD₃OD: δ (ppm) −0.01 (t, J=6.8 Hz, 1H), 0.61 (t, J=6.8 Hz, 1H), 0.68 (t, J=6.8 Hz, 1H), 1.03 (t, J=6.8 Hz, 1H), 7.09-7.14 (m, 3H), 7.27-7.47 (m, 9H).

### Example 225

### Synthesis of 4,6-bis(4-chlorophenyl)-7-hydroxy-7-(3-(trifluoromethyl)phenyl)-4,6-diazaspiro[2.4]heptan-5-one

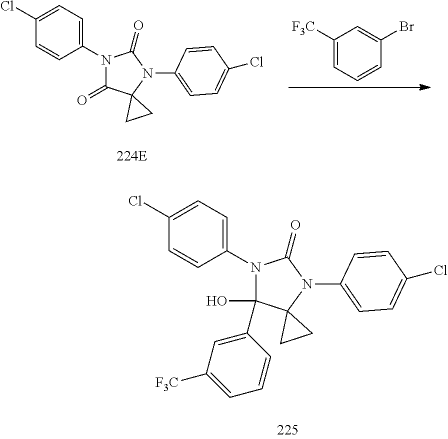 [SMILES: O=C1N(c2ccc(Cl)cc2)C(=O)C2(CC2)N1c1ccc(Cl)cc1 ; FC(F)(F)c1cccc(Br)c1 ; O=C1N(c2ccc(Cl)cc2)C2(CC2)C(O)(c2cccc(C(F)(F)F)c2)N1c1ccc(Cl)cc1]

Compound 225 was synthesized by employing the procedure described for Compound 127 using Compound 224E and 1-bromo-3-(trifluoromethyl)benzene in lieu of Compound 2C and 1-bromo-4-(tert-butyl)benzene. LC-MS (ESI) m/z: 493 [M+H]⁺. ¹H-NMR (400 MHz, CDCl₃): δ (ppm) −0.06-−0.02 (m, 1H), 0.49-0.54 (m, 1H), 0.65-0.70 (m, 1H), 0.97-1.01 (m, 1H), 4.75 (s, 1H), 7.06 (d, J=8.4 Hz, 2H), 7.15 (d, J=8.8 Hz, 2H), 7.26-7.41 (m, 5H), 7.50-7.56 (m, 2H), 7.72 (s, 1H).

### Example 226

### Synthesis of 3-(4,6-bis(4-chlorophenyl)-7-hydroxy-5-oxo-4,6-diazaspiro[2.4]heptan-7-yl)benzonitrile

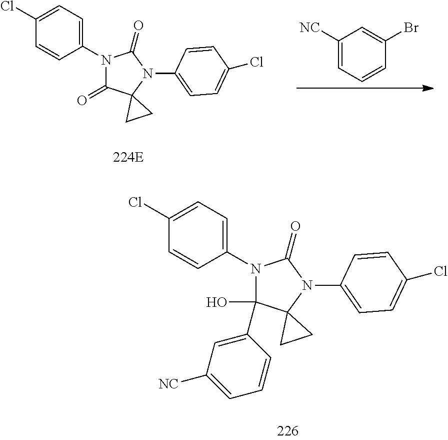 [SMILES: O=C1N(c2ccc(Cl)cc2)C(=O)C2(CC2)N1c1ccc(Cl)cc1 ; N#Cc1cccc(Br)c1 ; N#Cc1cccc(C2(O)N(c3ccc(Cl)cc3)C(=O)N(c3ccc(Cl)cc3)C23CC3)c1]

Compound 226 was synthesized by employing the procedure described for Compound 127 using Compound 224E and 3-bromobenzonitrile in lieu of Compound 2C and 1-bromo-4-(tert-butyl)benzene. LC-MS (ESI) m/z: 450 [M+H]⁺. ¹H-NMR (400 MHz, CDCl₃): δ (ppm) −0.07-−0.04 (m, 1H), 0.52-0.55 (m, 1H), 0.69-0.72 (m, 1H), 0.98-1.01 (m, 1H), 4.73 (s, 1H), 7.07 (d, J=8.4 Hz, 2H), 7.16 (d, J=6.8 Hz, 2H), 7.29-7.41 (m, 5H), 7.55 (d, J=7.6 Hz, 1H), 7.62 (d, J=8.4 Hz, 1H), 7.76 (s, 1H).

### Example 227

### Synthesis of 4,6-bis(4-chlorophenyl)-7-hydroxy-7-(3-hydroxyphenyl)-4,6-diazaspiro[2.4]heptan-5-one

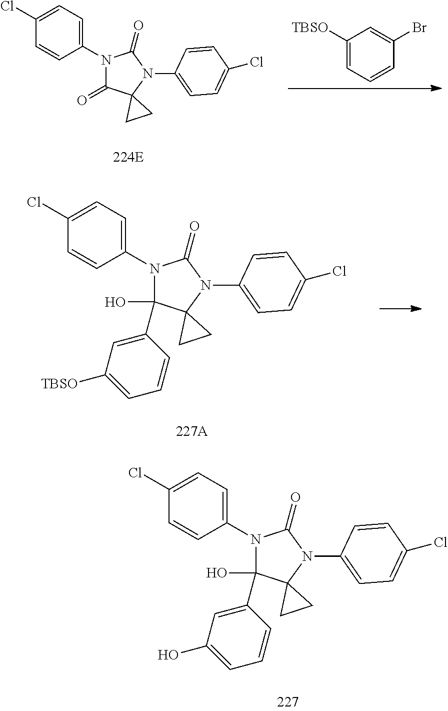 [SMILES: O=C1N(c2ccc(Cl)cc2)C(=O)C2(CC2)N1c1ccc(Cl)cc1 ; CC(C)(C)[Si](C)(C)Oc1cccc(Br)c1 ; CC(C)(C)[Si](C)(C)Oc1cccc(C2(O)N(c3ccc(Cl)cc3)C(=O)N(c3ccc(Cl)cc3)C23CC3)c1 ; O=C1N(c2ccc(Cl)cc2)C2(CC2)C(O)(c2cccc(O)c2)N1c1ccc(Cl)cc1]

Compound 227A was synthesized by employing the procedure described for Compound 127 using Compound 224E and (3-bromophenoxy)(tert-butyl)dimethylsilane in lieu of Compound 2C and 1-bromo-4-(tert-butyl)benzene. LC-MS (ESI) m/z: non-ionizable compound under routine conditions used.

To a stirred solution of Compound 227A (310 mg, 0.58 mmol) in THF (5 mL) was added TBAF (207 mg, 0.793 mmol) at room temperature and stirred at room temperature for 30 minutes. The reaction mixture was diluted with ethyl acetate (100 mL), washed with water (100 mL×3), dried over anhydrous Na₂SO₄, filtered, and concentrated under vacuum. The residue was purified with preparative HPLC to give Compound 227. LC-MS (ESI) m/z: 441 [M+H]⁺. ¹H-NMR (400 MHz, CDCl₃): δ (ppm) 0.07-0.13 (m, 1H), 0.44-0.51 (m, 1H), 0.58-0.63 (m, 1H), 1.00-1.06 (m, 1H), 4.24 (s, 1H), 6.29 (s, 1H), 6.67 (dd, J₁=1.6 Hz, J₂=8.0 Hz, 1H), 6.89-6.92 (m, 2H), 7.03-7.20 (m, 6H), 7.45 (d, J=8.4 Hz, 2H).

### Example 228

### Synthesis of 7-(3-chlorophenyl)-4,6-bis(4-chlorophenyl)-7-hydroxy-4,6-diazaspiro[24]heptan-5-one

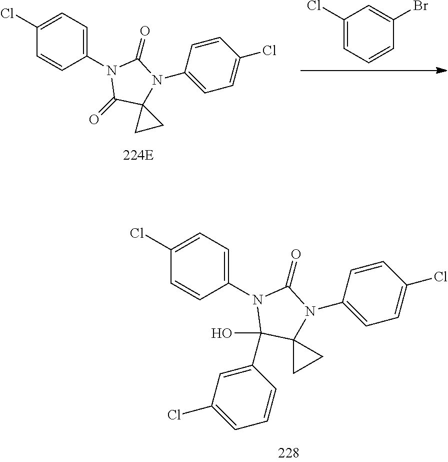 [SMILES: O=C1N(c2ccc(Cl)cc2)C(=O)C2(CC2)N1c1ccc(Cl)cc1 ; Clc1cccc(Br)c1 ; O=C1N(c2ccc(Cl)cc2)C2(CC2)C(O)(c2cccc(Cl)c2)N1c1ccc(Cl)cc1]

Compound 228 was synthesized by employing the procedure described for Compound 127 using Compound 224E and 1-bromo-3-chlorobenzene in lieu of Compound 2C and 1-bromo-4-(tert-butyl)benzene. LC-MS (ESI) m/z: 459 [M+H]⁺. ¹H-NMR (400 MHz, Acetone-d₆): δ (ppm) 0.02-0.05 (m, 1H), 0.59-0.65 (m, 1H), 0.78-0.85 (m, 1H), 1.11-1.17 (m, 1H), 6.89 (s, 1H), 7.21-7.23 (m, 2H), 7.30-7.41 (m, 4H), 7.48-7.53 (m, 3H), 7.59-7.62 (m, 3H).

### Example 229

### Synthesis of 3-(4-bromophenyl)-4-hydroxy-5-methyl-1-phenyl-4-(3-(trifluoromethoxy)phenyl)imidazolidin-2-one

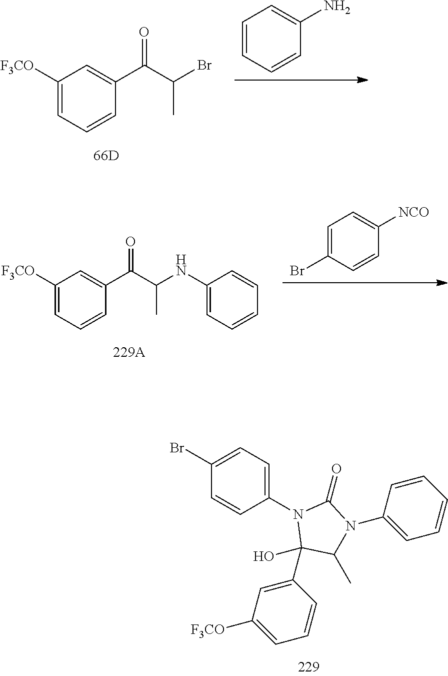

Compounds 229A and 229 were synthesized by employing the procedures described for Compound 1B and 1 using aniline, Compounds 66D, at 60° C., and 229A in lieu of 4-bromoaniline, Compound 1A, at 25° C., and Compound 1B. Compound 229A. LC-MS (ESI) m/z: 310 [M+H]⁺; ¹H-NMR (CDCl₃, 400 MHz): δ (ppm) 1.50 (d, J=7.2 Hz, 3H), 4.64 (s, 1H), 5.09 (q, J=6.8 Hz, 1H), 6.69 (d, J=8.0 Hz, 2H), 6.75 (d, J=7.2 Hz, 1H), 7.20 (t, J=8 Hz, 2H), 7.48 (d, J=8.0 Hz, 1H), 7.58 (t, J=8.0 Hz, 1H), 7.88 (s, 1H), 7.96 (d, J=7.6 Hz, 1H). Compound 229. LC-MS (ESI) m/z: 507 [M+H]⁺; ¹H-NMR (CDCl₃, 400 MHz): δ (ppm) 0.66 (d, J=6.4 Hz, 3H), 4.30 (q, J=6.8 Hz, 1H), 4.79 (s, 1H), 7.11-7.16 (m, 2H), 7.20-7.37 (m, 11H).

### Example 230

### Synthesis of 4-hydroxy-5-methyl-1,3-diphenyl-4-(3-(trifluoromethoxy)phenyl)imidazolidin-2-one, (5S)-4-hydroxy-5-methyl-1,3-diphenyl-4-(3-(trifluoromethoxy)phenyl)imidazolidin-2-one, and (5R)-4-hydroxy-5-methyl-1,3-diphenyl-4-(3-(trifluoromethoxy)phenyl)imidazolidin-2-one

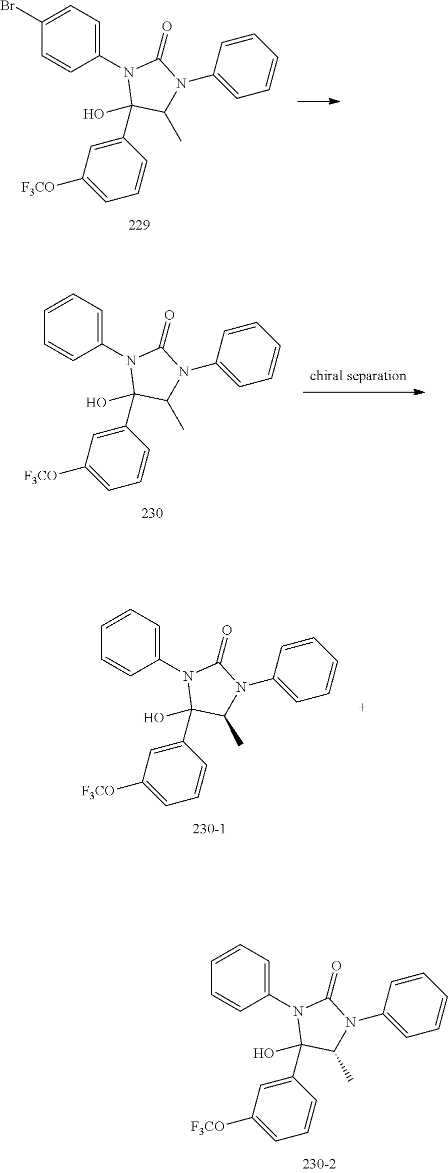 [SMILES: CC1N(c2ccccc2)C(=O)N(c2ccc(Br)cc2)C1(O)c1cccc(OC(F)(F)F)c1 ; CC1N(c2ccccc2)C(=O)N(c2ccccc2)C1(O)c1cccc(OC(F)(F)F)c1 ; C[C@@H]1N(c2ccccc2)C(=O)N(c2ccccc2)C1(O)c1cccc(OC(F)(F)F)c1 ; C[C@H]1N(c2ccccc2)C(=O)N(c2ccccc2)C1(O)c1cccc(OC(F)(F)F)c1]

A mixture of Compound 229 (400 mg, 0.79 mmol), 10% Pd/C (40 mg), and triethylamine (398 mg, 3.95 mmol) in MeOH (20 mL) was stirred at room temperature under hydrogen (1 atm.) overnight. The mixture was filtered and the filtrate was concentrated. The residue was purified with preparative HPLC to yield Compound 230. LC-MS (ESI) m/z: 429 [M+H]⁺; ¹H-NMR (CDCl₃, 400 MHz): δ (ppm) 0.80, 1.29 (d, J=6.4 Hz, 3H), 3.23, 3.63 (s, 1H), 4.29, 4.41 (q, J=6.4 Hz, 1H), 7.06-7.29 (m, 5H), 7.31-7.57 (m, 9H).

Compound 230 was separated with chiral HPLC to give two isomers Compounds 230-1 and 230-2. Compound 230-1: LC-MS (ESI) m/z: 429 [M+H]⁺; ¹H-NMR (CDCl₃, 400 MHz): δ (ppm) 0.79, 1.29 (d, J=6.4 Hz, 3H), 3.35 (s, 1H), 4.29, 4.39 (q, J=6.4 Hz, 1H), 7.06-7.25 (m, 5H), 7.33-7.55 (m, 9H); Chiral separation condition: MeOH contained 0.2% Ammonia; OD (4.6*100 mm, 5 μm); retention time: 0.62 minute (79.5%), 1.33 minute (20.5%). Compound 230-1: LC-MS (ESI) m/z: 429 [M+H]⁺; ¹H-NMR (CDCl₃, 400 MHz): δ (ppm) 0.80, 1.29 (d, J=6.4 Hz, 3H), 3.32, 3.74 (s, 1H), 4.29, 4.40 (q, J=6.4 Hz, 1H), 7.06-7.25 (m, 5H), 7.33-7.56 (m, 9H); Chiral separation condition: MeOH contained 0.2% Ammonia; OD (4.6*100 mm, 5 μm); retention time: 1.06 minute (21.5%), 1.99 minute (78.5%).

### Example 231

### Synthesis of 3-(1,3-bis(4-chlorophenyl)-5-hydroxy-4-isopropyl-2-oxoimidazolidin-4-yl)benzonitrile

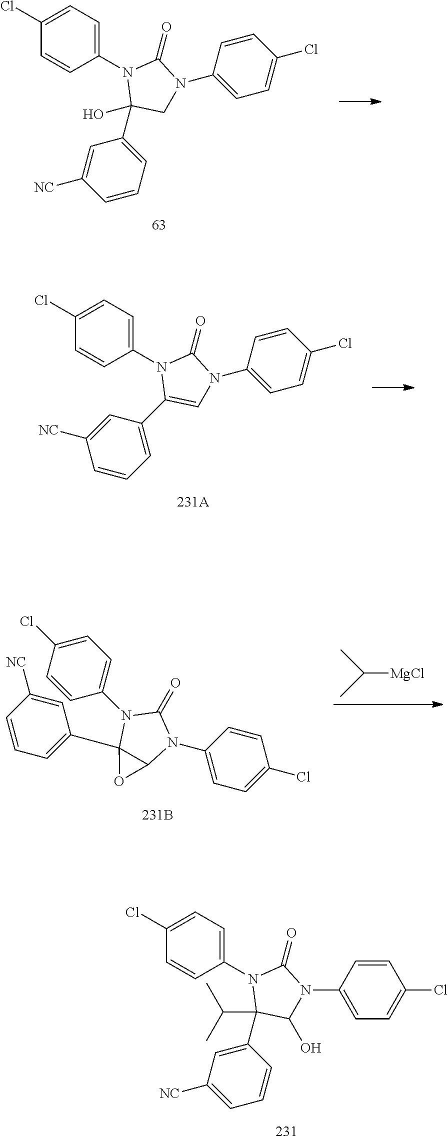

Compounds 231A, 231B, and 231 were synthesized by employing the procedures described for Compounds 209A, 86B, and 86 using Compounds 63, 231A, 231B and isopropylmagnesium chloride in lieu of Compounds 1, 86A, and 86B and phenylmagnesium bromide. Compound 231A. LC-MS (ESI) m/z: 406 [M+H]⁺; ¹H-NMR (DMSO-d₆, 400 MHz): δ (ppm) 7.27-7.33 (m, 3H), 7.47-7.60 (m, 5H), 7.72-7.76 (m, 2H), 7.82 (s, 1H), 7.87-7.89 (m, 2H).

Compound 231B. LC-MS (ESI) m/z: 422 [M+H]⁺. Compound 231. LC-MS (ESI) m/z: 466[M+H]⁺; ¹H-NMR (acetone-d₆, 400 MHz): δ (ppm) 1.06 (d, J=7.2 Hz, 3H), 1.19 (d, J=6.8 Hz, 3H), 2.19-2.26 (m, 1H), 5.14 (s, 1H), 5.74 (s, 1H), 7.24 (d, J=9.2 Hz, 2H), 7.43 (d, J=8.8 Hz, 2H), 7.56-7.69 (m, 5H), 7.71-7.82 (m, 1H), 7.84-7.88 (m, 2H).

### Example 232

### Synthesis of 3-(1,3-bis(4-bromophenyl)-5-hydroxy-4-isopropyl-2-oxoimidazolidin-4-yl)benzonitrile

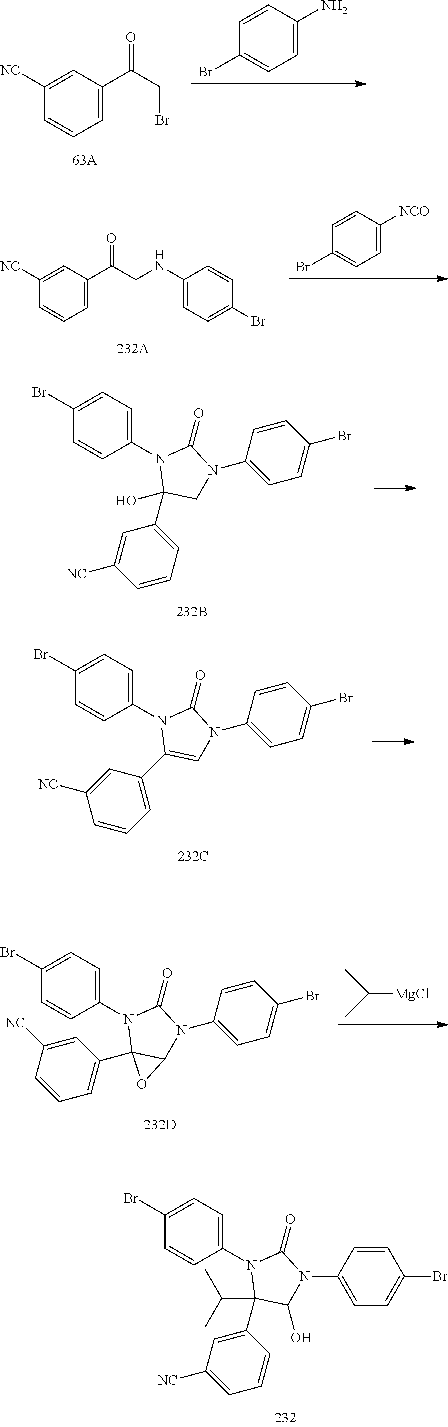

Compounds 232A, 232B, 232C, 232D, and 232 were synthesized by employing the procedures described for Compounds 1B, 12, 209A, 86B, and 86 using Compounds 63A using NMP as solvent, 232A using NaHCO₃ as base, 1-bromo-4-isocyanatobenzene, Compounds 232B, 232C, and Compound 232D and isopropylmagnesium chloride in lieu of Compounds 1A using EtOH as solvent, 12B using DIPEA as base, 1-chloro-4-isocyanatobenzene, Compounds 1, 86A, and Compound 86B and phenylmagnesium bromide. Compound 232A. LC-MS (ESI) m/z: 315 [M+H]⁺. Compound 232B. LC-MS (ESI) m/z: 512 [M+H]⁺. Compound 232C. LC-MS (ESI) m/z: 494 [M+H]⁺; ¹H-NMR (CDCl₃, 400 MHz): δ (ppm) 6.87 (s, 1H), 7.10-7.12 (m, 3H), 7.38-7.59 (m, 9H). Compound 232D. LC-MS (ESI) m/z: 510 [M+H]⁺; ¹H-NMR (CDCl₃, 400 MHz): δ (ppm) 5.65 (s, 1H), 7.33-7.69 (m, 12H). Compound 232. LC-MS (ESI) m/z: 554 [M+H]⁺; ¹H-NMR (acetone-d₆, 400 MHz): δ (ppm) 1.05 (d, J=7.2 Hz, 3H), 1.19 (d, J=6.4 Hz, 3H), 2.19-2.26 (m, 1H), 5.15 (s, 1H), 5.73 (s, 1H), 7.37-7.40 (m, 2H), 7.54-7.60 (m, 7H), 7.69-7.71 (m, 1H), 7.81-7.88 (m, 2H).

### Example 233

### Synthesis of 1,3-bis(4-chlorophenyl)-5-cyclopropyl-4-(3-(difluoromethoxy)phenyl)-4-hydroxyimidazolidin-2-one, (5S)-1,3-bis(4-chlorophenyl)-5-cyclopropyl-4-(3-(difluoromethoxy)phenyl)-4-hydroxyimidazolidin-2-one, and (5R)-1,3-bis(4-chlorophenyl)-5-cyclopropyl-4-(3-(difluoromethoxy)phenyl)-4-hydroxyimidazolidin-2-one

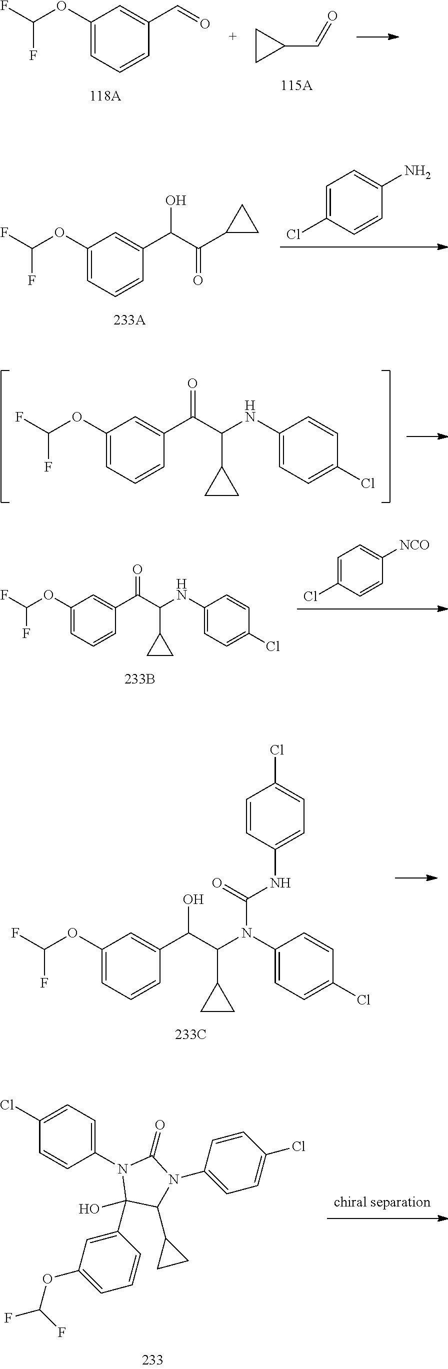

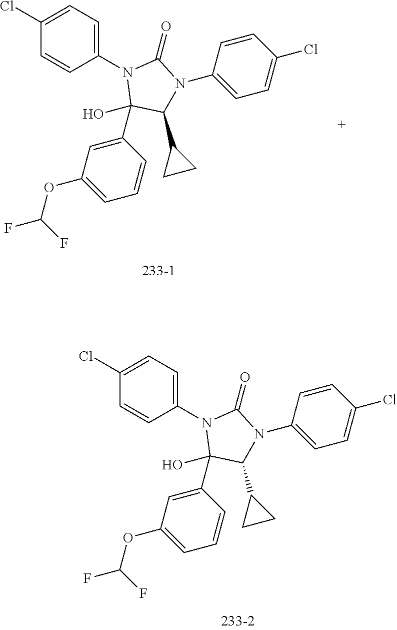 [SMILES: O=C1N(c2ccc(Cl)cc2)[C@@H](C2CC2)C(O)(c2cccc(OC(F)F)c2)N1c1ccc(Cl)cc1 ; O=C1N(c2ccc(Cl)cc2)[C@H](C2CC2)C(O)(c2cccc(OC(F)F)c2)N1c1ccc(Cl)cc1]

Compound 233A was synthesized by employing the procedure described for Compound 109C using Compounds 118A and 115A in lieu of Compounds 109B and 109A. LC-MS (ESI) m/z: 225 [M-OH]⁺.

A solution of Compound 233A (12.4 mmol) and 4-chloroaniline (2.14 g, 13.7 mmol) in ethanol (10 mL) in a sealed tube was stirred at 130° C. for 16 hours. The mixture was cooled down to room temperature and sodium borohydride (1.38 g, 37.2 mmol) was added. The reaction mixture was stirred at 5° C. for 1 hour, quenched with aqueous HCl solution (1 N, 50 mL), and extracted with ethyl acetate (50 mL×3). The combined extracts was washed with water (30 mL×2) and brine (30 mL), dried over anhydrous sodium sulfate, filtered, and concentrated. The residue was purified with flash column chromatography on silica gel (ethyl acetate in petroleum ether, 20% v/v) to give Compound 233B. Compound 233B. LC-MS (ESI) m/z: 354 [M+H]⁺; ¹H-NMR (CDCl₃, 400 MHz): δ (ppm) −0.11-−0.07 (m, 1H), 0.15-0.17 (m, 1H), 0.35-0.43 (m, 2H), 0.86-0.88 (m, 1H), 2.63-2.64 (m, 1H), 2.93-2.94 (m, 1H), 3.78-3.80 (m, 1H), 5.02 (s, 1H), 6.34-6.71 (m, 4H), 7.05-7.07 (m, 1H), 7.12-7.15 (m, 2H), 7.24 (s, 1H), 7.34-7.36 (m, 1H).

Compounds 233C and 233 were synthesized by employing the procedures described for Compounds 1 and 14 using Compounds 233B and 1-chloro-4-isocyanatobenzene, and Compound 233C in lieu of Compounds 1B and 1-bromo-4-isocyanatobenzene, and Compound 14D. Compound 233C. LC-MS (ESI) m/z: 507 [M+H]⁺; ¹H-NMR (CDCl₃, 400 MHz): δ (ppm) 0.02-0.04 (m, 1H), 0.22-0.27 (m, 1H), 0.37-0.42 (m, 1H), 0.51-0.57 (m, 1H), 0.96-1.00 (m, 1H), 3.50 (d, J=10.4 Hz, 1H), 4.47 (d, J=4.0 Hz, 1H), 5.33 (s, 1H), 5.89 (s, 1H), 6.32-6.6.69 (m, 1H), 6.98-7.07 (m, 3H), 7.18-7.24 (m, 4H), 7.29-7.37 (m, 5H). Compound 233. LC-MS (ESI) m/z: 505 [M+H]⁺; ¹H-NMR (CD₃OD, 400 MHz): δ (ppm) −0.30-0.01 (m, 2H), 0.33-0.52 (m, 2H), 1.11-1.19 (m, 1H), 3.56-3.65 (m, 1H), 6.70-7.15 (m, 1H), 7.28-7.33 (m, 1H), 7.43-7.45 (m, 2H), 7.53-7.64 (m, 9H).

Compound 233 was separated with chiral HPLC to give Compound 233-1 and Compound 233-2. Compound 233-1: LC-MS (ESI) m/z: 505 [M+H]⁺; ¹H-NMR (CD₃OD, 400 MHz): δ (ppm) −0.41-−0.38 (m, 1H), −0.12-−0.10 (m, 1H), 0.33-0.41 (m, 2H), 1.03-1.08 (m, 1H), 3.47 (d, J=10.0 Hz, 1H), 6.59-6.96 (m, 1H), 7.02-7.04 (m, 1H), 7.17-7.20 (m, 2H), 7.30-7.34 (m, 1H), 7.42-7.47 (m, 8H). Chiral separation condition: MeOH contained 0.2% Methanol Ammonia; OJ-H (4.6*100 mm, 5 m); retention time: 1.55 minute (65%), 3.37 minute (35%).

Compound 233-2: LC-MS (ESI) m/z: 505 [M+H]⁺; ¹H-NMR (CD₃OD, 400 MHz): δ (ppm) −0.39-−0.38 (m, 1H), −0.11-−0.10 (m, 1H), 0.33-0.41 (m, 2H), 1.03-1.08 (m, 1H), 3.47 (d, J=10.0 Hz, 1H), 6.59-6.96 (m, 1H), 7.02-7.05 (m, 1H), 7.17-7.20 (m, 2H), 7.30-7.34 (m, 1H), 7.41-7.48 (m, 8H). Chiral separation condition: MeOH contained 0.2% Methanol Ammonia; OJ-H (4.6*100 mm, 5 μm); retention time: 2.07 minute (69%), 2.96 minute (31%).

### Example 234

### Synthesis of 1,3-bis(4-chlorophenyl)-4-hydroxy-4-(3-(trifluoromethoxy)phenyl)-7-oxa-1,3-diazaspiro[4.4]nonan-2-one, (4R,5S)-1,3-bis(4-chlorophenyl)-4-hydroxy-4-(3-(trifluoromethoxy)phenyl)-7-oxa-1,3-diazaspiro[4.4]nonan-2-one, (4S,5S)-1,3-bis(4-chlorophenyl)-4-hydroxy-4-(3-(trifluoromethoxy)phenyl)-7-oxa-1,3-diazaspiro[4.4]nonan-2-one, (4R,5R)-1,3-bis(4-chlorophenyl)-4-hydroxy-4-(3-(trifluoromethoxy)phenyl)-7-oxa-1,3-diazaspiro[4.4]nonan-2-one, and (4S,5R)-1,3-bis(4-chlorophenyl)-4-hydroxy-4-(3-(trifluoromethoxy)phenyl)-7-oxa-1,3-diazaspiro[4.4]nonan-2-one

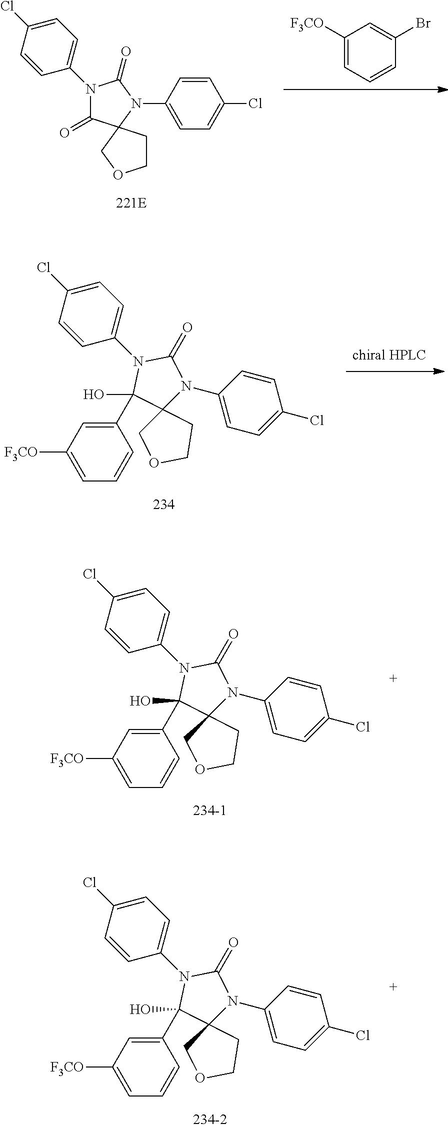 [SMILES: O=C1N(c2ccc(Cl)cc2)C(=O)C2(CCOC2)N1c1ccc(Cl)cc1 ; FC(F)(F)Oc1cccc(Br)c1 ; O=C1N(c2ccc(Cl)cc2)C2(CCOC2)C(O)(c2cccc(OC(F)(F)F)c2)N1c1ccc(Cl)cc1 ; O=C1N(c2ccc(Cl)cc2)[C@]2(CCOC2)[C@](O)(c2cccc(OC(F)(F)F)c2)N1c1ccc(Cl)cc1 ; O=C1N(c2ccc(Cl)cc2)[C@]2(CCOC2)[C@@](O)(c2cccc(OC(F)(F)F)c2)N1c1ccc(Cl)cc1]

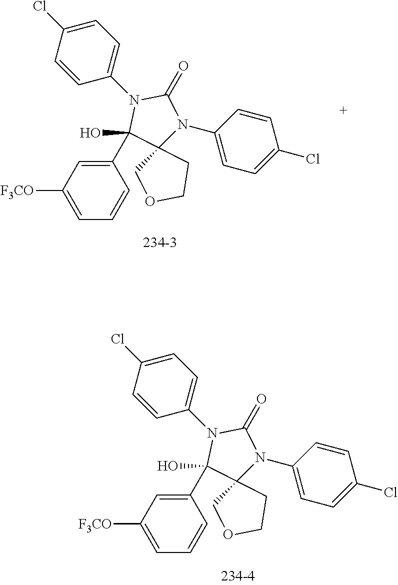 [SMILES: O=C1N(c2ccc(Cl)cc2)[C@@]2(CCOC2)[C@](O)(c2cccc(OC(F)(F)F)c2)N1c1ccc(Cl)cc1 ; O=C1N(c2ccc(Cl)cc2)[C@@]2(CCOC2)[C@@](O)(c2cccc(OC(F)(F)F)c2)N1c1ccc(Cl)cc1]

Compound 234 was synthesized by employing the procedure described for Compound 125 using Compound 221E and 1-bromo-3-(trifluoromethoxy)benzene in lieu of Compound 2C and 3-bromo-N,N-dimethylaniline. LC-MS (ESI) m/z: 539 [M+H]⁺. ¹H-NMR (400 MHz, CDCl₃): δ (ppm) 1.85-2.17 (m, 1H), 2.57-3.02 (m, 1H), 3.21-3.24 (m, 3H), 3.69-4.23 (m, 1H), 4.71-4.94 (m, 1H), 7.15-7.20 (m, 5H), 7.23-7.35 (m, 7H).

Compound 234 was separated by using chiral HPLC to give Compound 234-1, Compound 234-2, Compound 234-3, and Compound 234-4. Compound 234-1: LC-MS (ESI) m/z: 539 [M+H]⁺. ¹H-NMR (400 MHz, CDCl₃): δ (ppm) 2.14-2.21 (m, 1H), 2.61-2.68 (m, 1H), 3.27-3.35 (m, 2H), 3.52-3.57 (m, 1H), 3.75 (d, J=9.6 Hz, 1H), 4.01 (s, 1H), 7.17-7.20 (m, 3H), 7.27-7.39 (m, 9H). Chiral separation conditions: MeOH contained 0.2% NH₄OH; cellulose-SC (100×4.6 mm, 5 μm); retention time: 0.97 minutes. Compound 234-2: LC-MS (ESI) m/z: 539 [M+H]⁺. ¹H-NMR (400 MHz, CDCl₃): δ (ppm) 1.73-1.80 (m, 1H), 1.92-1.99 (m, 1H), 3.02-3.08 (m, 1H), 3.29-3.35 (m, 1H), 4.11 (d, J=10.8 Hz, 1H), 4.29 (d, J=10.8 Hz, 1H), 7.15-7.21 (m, 3H), 7.28-7.40 (m, 9H). Chiral separation conditions: MeOH contained 0.2% NH₄OH; cellulose-SC (100×4.6 mm, 5 μm); retention time: 1.29 minutes. Compound 234-3: LC-MS (ESI) m/z: 539 [M+H]⁺. ¹H-NMR (400 MHz, CDCl₃): δ (ppm) 1.71-1.78 (m, 1H), 1.90-1.97 (m, 1H), 3.01-3.07 (m, 1H), 3.28-3.33 (m, 1H), 4.10 (d, J=10.8 Hz, 1H), 4.28 (d, J=11.2 Hz, 1H), 7.14-7.21 (m, 3H), 7.26-7.39 (m, 9H). Chiral separation conditions: MeOH contained 0.2% NH₄OH; cellulose-SC (100×4.6 mm, 5 μm); retention time: 2.13 minutes. Compound 234-4: LC-MS (ESI) m/z: 539 [M+H]⁺. ¹H-NMR (400 MHz, CDCl₃): δ (ppm) 2.14-2.21 (m, 1H), 2.61-2.69 (m, 1H), 3.28-3.35 (m, 2H), 3.52-3.57 (m, 1H), 3.75 (d, J=10.0 Hz, 1H), 4.03 (s, 1H), 7.17-7.20 (m, 3H), 7.27-7.39 (m, 9H). Chiral separation conditions: MeOH contained 0.2% NH₄OH; cellulose-SC (100×4.6 mm, 5 μm); retention time: 3.84 minutes.

### Example 235

### Synthesis of 4-hydroxy-5-isopropyl-1,3-di-p-tolyl-4-(3-(trifluoromethoxy)phenyl)imidazolidin-2-one, (5S)-4-hydroxy-5-isopropyl-1,3-di-p-tolyl-4-(3-(trifluoromethoxy)phenyl)imidazolidin-2-one, and (5R)-4-hydroxy-5-isopropyl-1,3-di-p-tolyl-4-(3-(trifluoromethoxy)phenyl)imidazolidin-2-one

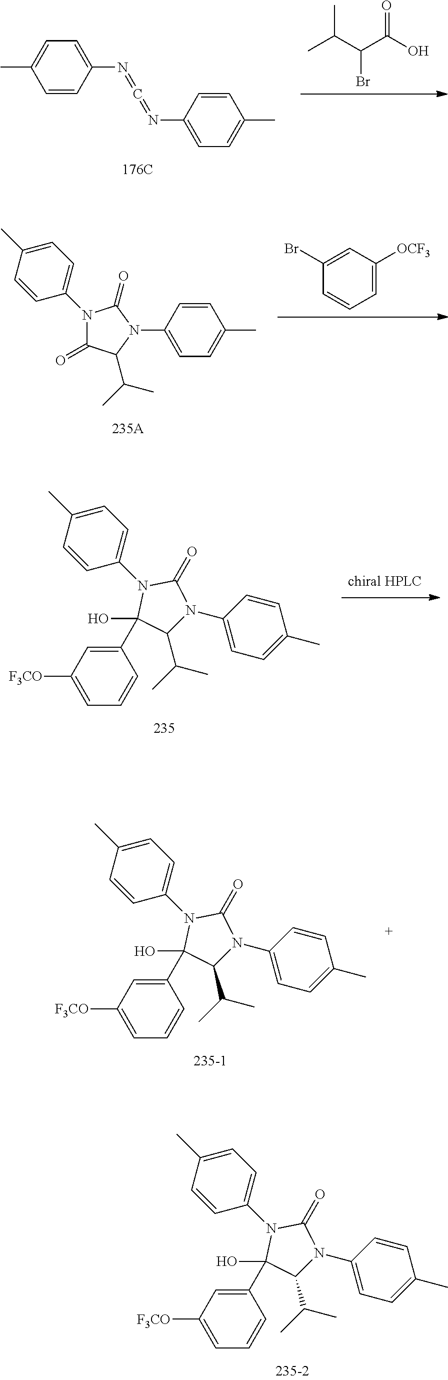

Compounds 235A and 235 were synthesized by employing the procedures described for Compounds 120D and 125 using 2-bromo-3-methylbutanoic acid, Compounds 176C using Na₂CO₃ as base and MeCN as solvent, 235A, and 1-bromo-3-(trifluoromethoxy)benzene in lieu of 2-bromo-2-methylpropanoic acid, Compound 120C using NaOH as base and 1,4-dioxane as solvent, 2C, and 3-bromo-N,N-dimethylaniline. Compound 235A. LC-MS (ESI) m/z: 323 [M+H]⁺; (CDCl₃, 400 MHz): δ (ppm) 0.91 (d, J=7.2 Hz, 3H), 1.23 (d, J=7.2 Hz, 3H), 2.25-2.33 (m, 1H), 2.36 (s, 3H), 2.38 (s, 3H), 4.57 (d, J=2.8 Hz, 1H), 7.22-7.30 (m, 6H), 7.33 (d, J=8.0 Hz, 2H). Compound 235. LC-MS (ESI) m/z: 485 [M+H]⁺; ¹H-NMR (CDCl₃, 400 MHz): δ (ppm) 0.96 (d, J=7.2 Hz, 3H), 1.01 (d, J=7.2 Hz, 3H), 2.25 (s, 3H), 2.31-2.34 (m, 1H), 2.36 (s, 3H), 2.96 (s, J=5.2 Hz, 1H), 4.23 (d, J=3.6 Hz, 1H), 7.02 (d, J=8.4 Hz, 2H), 7.12 (d, J=8.0 Hz, 2H), 7.20 (d, J=8.4 Hz, 2H), 7.25-7.53 (m, 6H).

Compound 235 was separated by using chiral HPLC to give Compound 235-1 and Compound 235-2. Compound 235-1: LC-MS (ESI) m/z: 485 [M+H]⁺; ¹H-NMR (CDCl₃, 400 MHz): δ (ppm) 0.97 (d, J=6.8 Hz, 3H), 1.01 (d, J=6.8 Hz, 3H), 2.25 (s, 3H), 2.31-2.34 (m, 1H), 2.36 (s, 3H), 3.01 (s, 1H), 4.23 (s, J=4.0 Hz, 1H) 7.02 (d, J=8.4 Hz, 2H), 7.12 (d, J=8.8 Hz, 2H), 7.18-7.53 (m, 8H). Chiral separation conditions: MeOH contained 0.2% Methanol Ammonia; Enantio PaK OD (4.6*100 mm, 5 μm); retention time: 2.01 minutes. Compound 235-2: LC-MS (ESI) m/z: 485[M+H]⁺; ¹H-NMR (CDCl₃, 400 MHz): δ (ppm) 0.97 (d, J=7.2 Hz, 3H), 1.01 (d, J=7.2 Hz, 3H), 2.25 (s, 3H), 2.31-2.34 (m, 1H), 2.36 (s, 3H), 2.98 (s, 1H), 4.23 (d, J=3.6 Hz, 1H) 7.02 (d, J=8.0 Hz, 2H), 7.12 (d, J=8.4 Hz, 2H), 7.18-7.53 (m, 8H). Chiral separation conditions: MeOH contained 0.2% Methanol Ammonia; Enantio PaK OD (4.6*100 mm, 5 μm); retention time: 2.99 minutes.

### Example 236

### Synthesis of 4,6-bis(4-chlorophenyl)-7-(3-(difluoromethoxy)phenyl)-7-hydroxy-4,6-diazaspiro[2.4]heptan-5-one

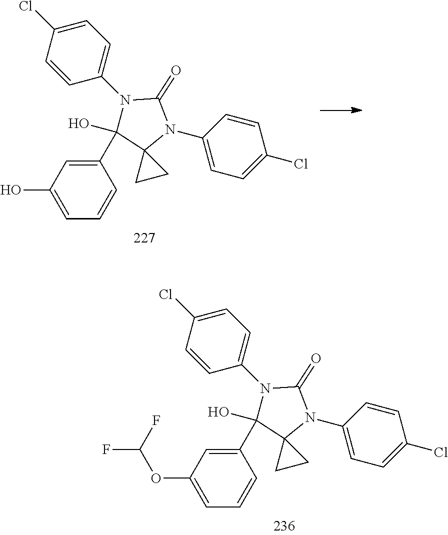 [SMILES: O=C1N(c2ccc(Cl)cc2)C2(CC2)C(O)(c2cccc(O)c2)N1c1ccc(Cl)cc1 ; O=C1N(c2ccc(Cl)cc2)C2(CC2)C(O)(c2cccc(OC(F)F)c2)N1c1ccc(Cl)cc1]

To a stirred solution of Compound 227 (150 mg, 0.341 mmol) in acetonitrile (3 mL) was added 2-chloro-2,2-difluoro-1-phenylethanone (650 mg, 3.41 mmol) and 30% KOH solution (3 mL). The mixture was stirred at 80° C. for 4 hours. After cooling down to room temperature, the mixture was diluted with water (100 mL) and extracted with ethyl acetate (50 mL×3). The combined extracts were dried over anhydrous Na₂SO₄, filtered, and concentrated under reduced pressure. The residue was purified with preparative HPLC to furnish Compound 236: LC-MS (ESI) m/z: 491 [M+H]⁺. ¹H-NMR (400 MHz, CDCl₃): δ (ppm) 0.01-0.07 (m, 1H), 0.47-0.53 (m, 1H), 0.64-0.71 (m, 1H), 0.96-1.03 (m, 1H), 4.48 (s, 1H), 6.43 (t, J=74.0 Hz, 1H), 6.99-7.01 (m, 1H), 7.06 (d, J=8.8 Hz, 2H), 7.16 (d, J=8.8 Hz, 2H), 7.20-7.30 (m, 5H), 7.35 (d, J=8.8 Hz, 2H).

### Example 237

### Synthesis of 1,3-bis(4-chlorophenyl)-4-hydroxy-5-(oxetan-3-yl)-4-(3-(trifluoromethoxy)phenyl)imidazolidin-2-one, (5S)-1,3-bis(4-chlorophenyl)-4-hydroxy-5-(oxetan-3-yl)-4-(3-(trifluoromethoxy)phenyl)imidazolidin-2-one, and (5R)-1,3-bis(4-chlorophenyl)-4-hydroxy-5-(oxetan-3-yl)-4-(3-(trifluoromethoxy)phenyl)imidazolidin-2-one

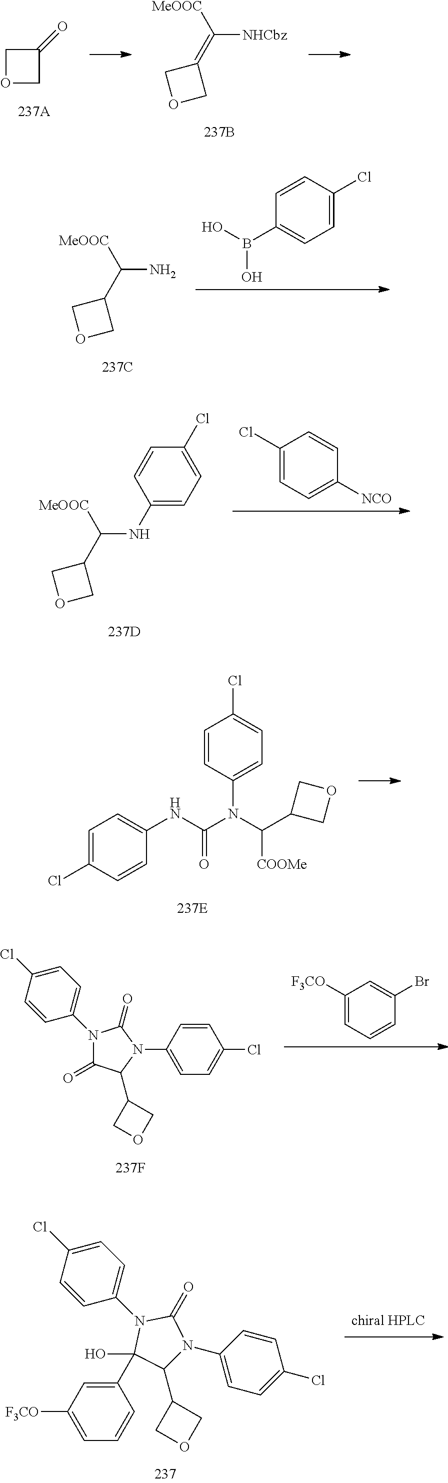

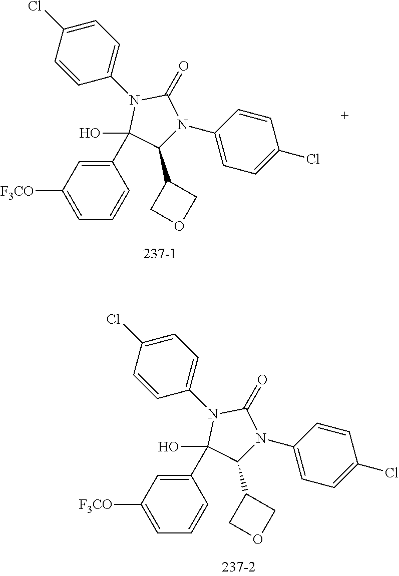 [SMILES: O=C1N(c2ccc(Cl)cc2)[C@@H](C2COC2)C(O)(c2cccc(OC(F)(F)F)c2)N1c1ccc(Cl)cc1 ; O=C1N(c2ccc(Cl)cc2)[C@H](C2COC2)C(O)(c2cccc(OC(F)(F)F)c2)N1c1ccc(Cl)cc1]

To a stirred suspension of NaH (60% suspension in oil, 2.78 g, 69.4 mmol) in dry DMF (80 mL) was added methyl 2-(benzyloxycarbonylamino)-2-(diethoxyphosphoryl) acetate (25 g, 69.4 mmol) in three portions at 0° C. and stirred at room temperature for 30 minutes. To the suspension was dropped a solution of oxetan-3-one, Compound 237A, (5 g, 69.4 mmol) in dry DMF (10 mL) at room temperature. The mixture was stirred at 45° C. overnight. The mixture was poured into water (200 mL) and a solid was precipitated. The solid was collected by filtration, washed with water (50 mL), and dried in vacuum to yield Compound 237B: LC-MS (ESI) m/z: 278 [M+H]⁺; ¹H-NMR (CDCl₃, 400 MHz): δ (ppm) 3.79 (s, 3H), 5.10 (s, 2H), 5.39-5.45 (m, 4H), 6.78 (s, 1H), 7.33-7.39 (m, 5H).

To a solution of Compound 237B (15.6 g, 56.3 mmol) in methanol (600 mL) was added Pd/C (1.6 g, 10% on carbon). The mixture was stirred at 80° C. under hydrogen atmosphere (1 atm.) overnight. After cooling down to room temperature, the mixture was filtered through Celite. The filtrate was concentrated to give a crude Compound 237C: LC-MS (ESI) m/z: 146 [M+H]⁺; ¹H-NMR (CDCl₃, 400 MHz): δ (ppm) 3.1-3.21 (m, 1H), 3.68-3.77 (m, 4H), 4.59-4.64 (m, 2H), 4.73-4.80 (m, 2H).

To a mixture of Compound 237C (6 g, 0.041 mol), 4-chlorophenylboronic acid (12.8 g, 0.082 mol), and cupric acetate (8.2 g, 0.045 mol) in dichloromethane (60 mL) was added triethylamine (8.28 g, 0.082 mol) and 4A molecular sieves (10 g) at room temperature. The mixture was stirred at room temperature for 4 days and filtered. The filtrate was concentrated under reduced pressure. The residue was purified with flash column chromatography on silica gel (ethyl acetate in petroleum ether from 0% to 40%) to give Compound 237D: LC-MS (ESI) m/z: 256 [M+H]⁺; ¹H-NMR (CDCl₃, 400 MHz): δ (ppm) 3.27-3.36 (m, 1H), 3.71 (s, 3H), 4.13 (d, J=10 Hz, 1H), 4.34 (t, J=10 Hz, 1H), 4.57-4.66 (m, 2H), 4.80 (q, J=6.8 Hz, 2H), 6.60 (d, J=8.8 Hz, 2H), 6.60 (d, J=8.8 Hz, 2H).

Compounds 237E, 237F, and 237 were synthesized by employing the procedures described for Compounds 224D, 224E, and 125 using Compounds 237D, 237E, 237F, and 3-bromobenzonitrile in lieu of Compounds 224C, 224D, 2C, and 3-bromo-N,N-dimethylaniline. Compound 237E. ¹H-NMR (CDCl₃, 400 MHz): δ (ppm) 3.41-3.50 (m, 1H), 3.76 (s, 3H), 4.54-4.57 (m, 3H), 4.71 (t, J=7.2 Hz, 1H), 5.15 (d, J=11 Hz, 1H), 5.99 (s, 1H), 7.18 (s, 4H), 7.33 (d, J=8.4 Hz, 2H), 746 (d, J=9.2 Hz, 2H). Compound 237F. LC-MS (ESI) m/z: 377 [M+H]⁺; ¹H-NMR (CDCl₃, 400 MHz): δ (ppm) 3.50-3.59 (m, 1H), 4.52 (t, J=7.2 Hz, 1H), 4.62 (t, J=6.4 Hz, 1H), 4.71-4.81 (m, 2H), 4.93 (d, J=8 Hz, 1H), 7.34 (d, J=9.2 Hz, 2H), 7.40-7.47 (m, 6H). Compound 237. LC-MS (ESI) m/z: 539 [M+H]⁺; ¹H-NMR (DMSO-d₆, 400 MHz): δ (ppm) 2.76-2.78, 3.14-3.15 (m, 1H), 3.51-3.98 (m, 3H), 4.15, 4.40 (t, J=5.2 Hz, 1H), 4.87 (d, J=7.2 Hz, 1H), 7.23-7.36 (m, 5H), 7.46-7.47 (m, 5H), 7.56-7.66 (m, 2H), 7.74, 8.16 (s, 1H).

Compound 237 was separated by using chiral HPLC to give Compound 237-1 and Compound 237-2. Compound 237-1: LC-MS (ESI) m/z: 539 [M+H]⁺; ¹H-NMR (DMSO-d₆, 400 MHz): δ (ppm) 2.76-2.79, 3.13-3.15 (m, 1H), 3.50-3.97 (m, 3H), 4.15, 4.40 (t, J=6.8 Hz, 1H), 4.85-4.89 (m, 1H), 7.23-7.34 (m, 5H), 7.46-7.48 (m, 5H), 7.50-7.64 (m, 2H), 7.74, 8.16 (s, 1H). Chiral separation conditions: MeOH contained 0.2% Methanol Ammonia; Cellulose-SC (4.6*100 mm, 5 μm); retention time: 1.43 minutes (77%), 2.29 minutes (22%). Compound 237-2: LC-MS (ESI) m/z: 539 [M+H]⁺; ¹H-NMR (DMSO-d₆, 400 MHz): δ (ppm) 2.76-2.79, 3.13-3.15 (m, 1H), 3.52-3.93 (m, 3H), 4.16-4.17, 4.38-4.42 (m, 1H), 4.85-4.89 (m, 1H), 7.23-7.34 (m, 5H), 7.46-7.48 (m, 5H), 7.50-7.66 (m, 2H), 7.87 (s, 1H). Chiral separation conditions: MeOH contained 0.2% Methanol Ammonia; Cellulose-SC (4.6*100 mm, 5 μm); retention time: 1.78 minutes (33%), 2.69 minutes (66%).

### Example 238

### Synthesis of 1,3-bis(4-chlorophenyl)-4-(3-(difluoromethoxy)phenyl)-4-hydroxy-5-(methoxymethyl)imidazolidin-2-one, (5S)-1,3-bis(4-chlorophenyl)-4-(3-(difluoromethoxy)phenyl)-4-hydroxy-5-(methoxymethyl)imidazolidin-2-one, and (5R)-1,3-bis(4-chlorophenyl)-4-(3-(difluoromethoxy)phenyl)-4-hydroxy-5-(methoxymethyl)imidazolidin-2-one

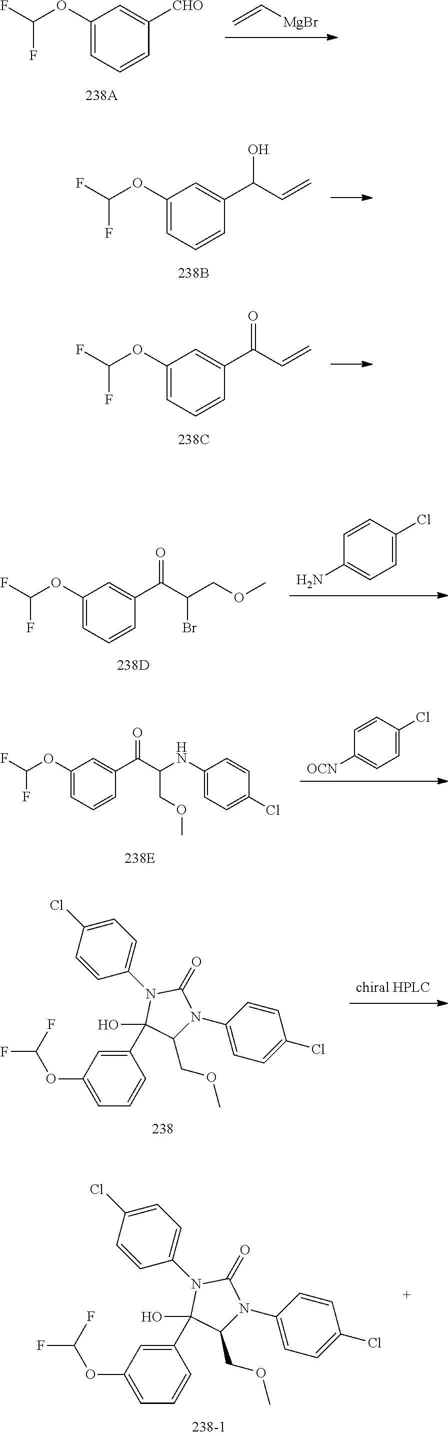

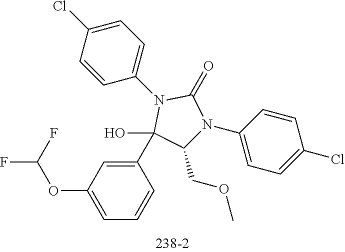 [SMILES: COC[C@H]1N(c2ccc(Cl)cc2)C(=O)N(c2ccc(Cl)cc2)C1(O)c1cccc(OC(F)F)c1]

To a solution of Compound 238A (1.72 g, 10 mmol) in THF (20 mL) was dropped a solution of vinylmagnesium bromide in THF (1.0 M, 15 mL, 15.0 mmol) at −60° C. under nitrogen atmosphere. The mixture was stirred at room temperature overnight, quenched with saturated ammonium chloride solution (20 mL), and extracted with ethyl acetate (20 mL×2). The combined extracts was washed with brine (20 mL), dried over anhydrous sodium sulfate, filtered, and evaporated to give a crude product, which was purified with column chromatography on silica gel (petroleum ether in ethyl acetate, 20% v/v) to afford Compound 238B: LC-MS (ESI) m/z: 183 [M-OH]⁺; ¹H-NMR (CDCl₃, 400 MHz): δ (ppm) 2.00 (d, J=3.6 Hz, 1H), 5.20-5.24 (m, 2H), 5.37 (d, J=17.2 Hz, 1H), 5.97-6.05 (m, 1H), 6.52 (t, J=74.0 Hz, 1H), 7.02-7.05 (m, 1H), 7.16 (s, 1H), 7.22 (d, J=7.6 Hz, 1H), 7.35 (t, J=8.0 Hz, 1H).

Compound 238C was synthesized by employing the procedure described for Compound 14 using Compound 238B in lieu of Compound 14D. Compound 238C. LC-MS (ESI) m/z: 199 [M+H]⁺; ¹H-NMR (CDCl₃, 400 MHz): δ (ppm) 5.98 (dd, J=10.8 Hz, J₂=1.6 Hz, 1H), 6.46 (dd, J₁=17.2 Hz, J₂=1.2 Hz, 1H), 6.57 (t, J=73.6 Hz, 1H), 7.08-7.15 (m, 1H), 7.33-7.36 (m, 1H), 7.50 (t, J=8.0 Hz, 1H), 7.69 (s, 1H), 7.78 (d, J=8.0 Hz, 1H).

To a solution of Compound 238C (792 mg, 4.0 mmol) in MeOH (10 mL) was added NBS (712 mg, 4.0 mmol) and concentrated H₂SO₄ (1 drop) at 0° C. The mixture was stirred at 0° C. for 2 hours, and then stirred at room temperature overnight. The reaction mixture was poured into a sodium thiosulfate solution (30 mL) and extracted with dichloromethane (30 mL×2). The combined extracts were washed with brine (30 mL), dried over anhydrous sodium sulfate, filtered, and evaporated to give a crude product, which was purified by column chromatography on silica gel (petroleum ether in ethyl acetate, 10% v/v) to furnish Compound 238D: LC-MS (ESI) m/z: 309 [M+H]⁺; ¹H-NMR (CDCl₃, 400 MHz): δ (ppm) 3.43 (s, 3H), 3.85-3.89 (m, 1H), 4.09-4.13 (m, 1H), 5.13-5.17 (m, 1H), 6.57 (t, J=72.8 Hz, 1H), 7.36-7.39 (m, 1H), 7.51 (t, J=8.0 Hz, 1H), 7.76 (s, 1H), 7.86 (d, J=7.6 Hz, 1H).

Compounds 238E and 238 were synthesized by employing the procedures described for Compounds 1B and 1 using Compounds 238D, 4-chloroaniline using NMP as solvent at 50° C., and 238E in lieu of Compounds 1A, 4-bromoaniline using EtOH as solvent at room temperature, and 1A. Compound 238E. LC-MS (ESI) m/z: 356 [M+H]⁺; ¹H-NMR (CDCl₃, 400 MHz): δ (ppm) 3.28 (s, 3H), 3.74 (d, J=4.0 Hz, 2H), 4.82-4.83 (m, 1H), 5.03-5.06 (m, 1H), 6.57 (t, J=72.8 Hz, 1H), 6.60-6.62 (m, 2H), 7.12-7.13 (m, 2H), 7.37-7.39 (m, 1H), 7.51 (t, J=8.0 Hz, 1H), 7.76 (s, 1H), 7.86 (d, J=8.0 Hz, 1H). Compound 238. LC-MS (ESI) m/z: 509 [M+H]⁺; ¹H-NMR (CDCl₃, 400 MHz): δ (ppm) 2.71, 3.30 (s, 3H), 3.60-6.63 (m, 1H), 3.83-3.86 (m, 1H), 4.24-4.25 (m, 1H), 5.74 (s, 1H), 6.48 (t, J=73.6 Hz, 1H), 7.03-7.17 (m, 3H), 7.23-7.26 (m, 2H), 7.31-7.40 (m, 7H).

Compound 238 was separated by using chiral HPLC to give Compound 238-1 and Compound 238-2. Compound 238-1: LC-MS (ESI) m/z: 509 [M+H]⁺; ¹H-NMR (CDCl₃, 400 MHz): δ (ppm) 3.31 (s, 3H), 3.62 (dd, J=10.8 Hz, J₂=2.8 Hz, 1H), 3.86 (d, J=10.8 Hz, 1H), 4.25 (d, J=2.8 Hz, 1H), 5.71 (s, 1H), 6.48 (t, J=73.1 Hz, 1H), 7.04-7.18 (m, 3H), 7.31-7.40 (m, 9H). Chiral separation conditions: MeOH contained 0.2% Methanol Ammonia; CC4 (4.6*100 mm, 5 μm); retention time: 1.15 minutes (92%), 1.52 minutes (7%).

Compound 238-2: LC-MS (ESI) m/z: 509 [M+H]⁺; ¹H-NMR (CDCl₃, 400 MHz): δ (ppm) 2.75, 3.31 (s, 3H), 3.62 (dd, J₁=10.8 Hz, J₂=2.8 Hz, 1H), 3.86 (d, J=10.8 Hz, 1H), 4.25 (d, J=2.8 Hz, 1H), 5.71 (s, 1H), 6.48 (t, J=73.6 Hz, 1H), 7.04-7.17 (m, 3H), 7.29-7.42 (m, 9H). Chiral separation conditions: MeOH contained 0.2% Methanol Ammonia; CC4 (4.6*100 mm, 5 μm); retention time: 1.46 minutes (26%), 2.38 minutes (72%).

### Example 239

### Synthesis of 8-hydroxy-5,7-di-p-tolyl-8-(3-(trifluoromethoxy)phenyl)-5,7-diazaspiro[3.4]octan-6-one

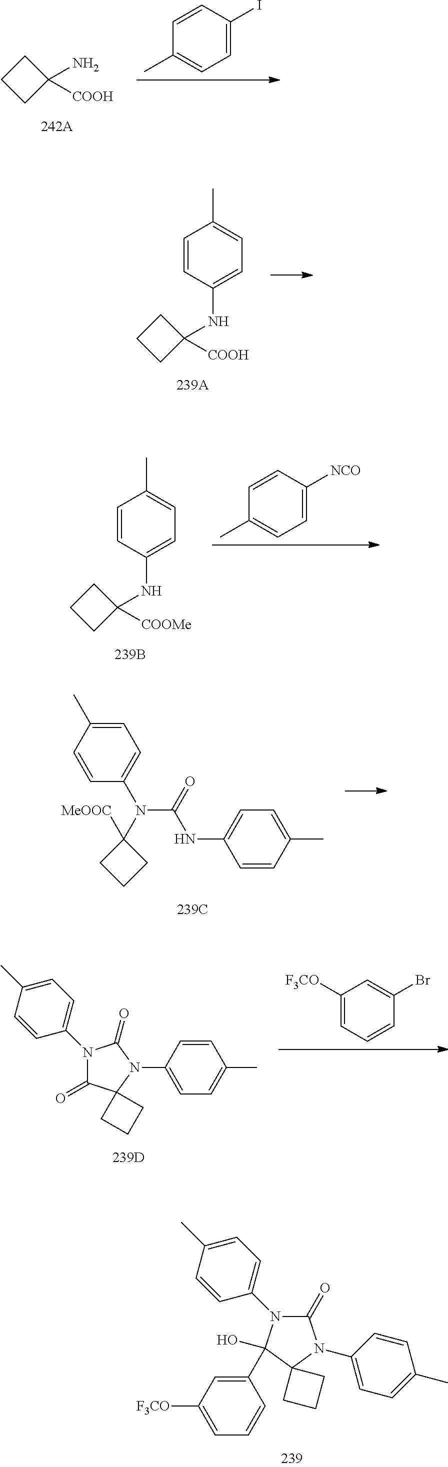

Compounds 239A, 239B, 239C, 239D, and 239 were synthesized by employing the procedures described for Compounds 224B, 224C, 224D, 224E, and 125 using 1-iodo-4-methylbenzene, Compounds 242A, 239A, 239B, 1-isocyanato-4-methylbenzene, 239C, 239D, and 1-bromo-3-(trifluoromethoxy)benzene in lieu of methyl 1-aminocyclopropane-1-carboxylate, Compounds 224A, 224B, 224C, 1-chloro-4-isocyanatobenzene, 224D, 2C, and 3-bromo-N,N-dimethylaniline. Compound 239A. LC-MS (ESI) m/z: 206 [M+H]⁺. Compound 239B. LC-MS (ESI) m/z: 220 [M+H]⁺. Compound 239C. LC-MS (ESI) m/z: 353 [M+H]⁺. Compound 239D. LC-MS (ESI) m/z: 321 [M+H]⁺. ¹H-NMR (400 MHz, CDCl₃): δ (ppm) 1.63-1.66 (m, 1H), 2.11-2.15 (m, 1H), 2.39-2.41 (m, 6H), 2.53-2.61 (m, 4H), 7.31-7.41 (m, 8H). Compound 239. LC-MS (ESI) m/z: 483 [M+H]⁺; ¹H-NMR (400 MHz, CDCl₃): δ (ppm) 0.80-0.87 (m, 1H), 1.04-1.09 (m, 1H), 1.70-1.75 (m, 1H), 2.00-2.08 (m, 1H), 2.21-2.23 (m, 4H), 2.38 (s, 3H), 2.66-2.72 (m, 1H), 3.88 (s, 1H), 6.97-6.99 (m, 2H), 7.13-7.34 (m, 8H), 7.45-7.49 (m, 2H).

### Example 240

### Synthesis of 5,7-bis(4-chlorophenyl)-2-fluoro-8-hydroxy-8-(3-(trifluoromethoxy)phenyl)-5,7-diazaspiro[3.4]octan-6-one

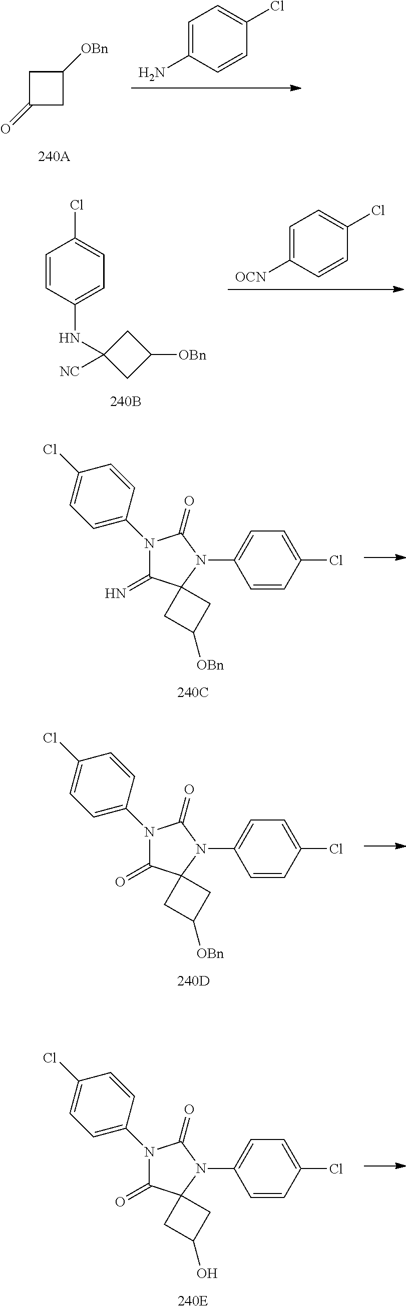

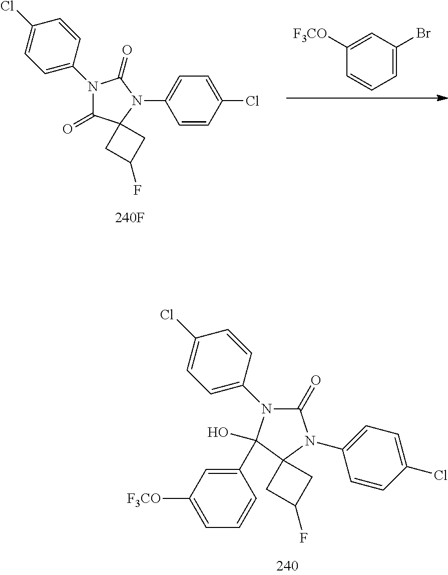 [SMILES: O=C1N(c2ccc(Cl)cc2)C(=O)C2(CC(F)C2)N1c1ccc(Cl)cc1 ; FC(F)(F)Oc1cccc(Br)c1 ; O=C1N(c2ccc(Cl)cc2)C2(CC(F)C2)C(O)(c2cccc(OC(F)(F)F)c2)N1c1ccc(Cl)cc1]

To a mixture of Compound 240A (352 mg, 2.0 mmol), 4-chloroaniline (254 mg, 2.0 mmol), 2,2,2-trifluoroethanol (1.0 mL), and sodium sulfate (2.0 g) in dichloromethane (10 mL) was dropped trimethylsilyl cyanide (396 mg, 4.0 mmol). The mixture was stirred at room temperature for 48 hours and filtered. The filtrate was concentrated under reduced pressure to give a crude product, which was purified by column chromatography (ethyl acetate in petroleum ether, 10% v/v) to yield Compound 240B: LC-MS (ESI) m/z: 313 [M+H]⁺; ¹H-NMR (400 MHz, CDCl₃): δ (ppm) 2.33-2.38 (m, 2H), 3.12-3.17 (m, 2H), 4.13 (s, 1H), 4.26-4.30 (m, 1H), 4.47 (s, 2H), 6.55-6.59 (m, 2H), 7.19-7.22 (m, 2H), 7.30-7.36 (m, 5H).

Compound 240C was synthesized by employing the procedure described for Compound 128C using Compound 240B in lieu of methyl Compound 128B. LC-MS (ESI) m/z: 466 [M+H]⁺.

To a solution of Compound 240C (50 mg, 0.11 mmol) in MeOH (5 mL) was added concentrated hydrochloric acid (0.5 mL). The mixture was stirred at 50° C. for 5 hours and concentrated under reduced pressure. The residue was purified by preparative TLC (ethyl acetate in petroleum ether, 10% v/v) to furnish Compound 240D: LC-MS (ESI) m/z: 467 [M+H]⁺; ¹H-NMR (400 MHz, CDCl₃): δ (ppm) 2.33-2.38 (m, 2H), 2.82-2.86 (m, 2H), 4.34-4.37 (m, 1H), 4.40 (s, 2H), 7.23-7.26 (m, 4H), 7.29-7.34 (m, 3H), 7.43 (s, 4H), 7.48-7.50 (m, 2H).

To a solution of Compound 240D (4.6 g, 10 mmol) in dichloromethane (100 mL) was dropped TMSI (4.0 g, 20 mmol). The mixture was stirred at room temperature overnight and quenched with saturated sodium hydrogen sulfite solution. The organic layer was dried over anhydrous sodium sulfate, filtered, and evaporated to give a crude product, which was purified with column chromatography on silica gel (ethyl acetate in petroleum ether, 30% v/v) to furnish Compound 240E: LC-MS (ESI) m/z: 377 [M+H]⁺. ¹H-NMR (400 MHz, CDCl₃): δ (ppm) 1.97 (d, J=5.6 Hz, 1H), 2.30-2.36 (m, 2H), 2.87-2.92 (m, 2H), 4.63-4.67 (m, 1H), 7.24-7.27 (m, 2H), 7.44-7.53 (m, 6H).

To a solution of Compound 240E (377 mg, 1.0 mmol) in dichloromethane (5 mL) was dropped DAST (483 mg, 3.0 mmol) at 0° C. The mixture was stirred at room temperature overnight and quenched with saturated sodium bicarbonate (3 mL). The organic layer was dried over anhydrous sodium sulfate, filtered, and evaporated to give a crude product, which was purified by flash column chromatography on silica gel (ethyl acetate in petroleum ether, 20% v/v) to afford Compound 240F: LC-MS (ESI) m/z: 379 [M+H]⁺; ¹H-NMR (400 MHz, CDCl₃): δ (ppm) 2.77-2.85 (m, 2H), 3.12-3.23 (m, 2H), 4.56-4.73 (m, 1H), 7.32-7.35 (m, 2H), 7.44-7.45 (m, 4H), 7.51-7.54 (m, 2H).

Compound 240 was synthesized by employing the procedure described for Compound 125 using Compound 240F and 1-bromo-3-(trifluoromethoxy)benzene in lieu of Compound 128B and 3-bromo-N,N-dimethylaniline. LC-MS (ESI) m/z: 541 [M+H]⁺; ¹H-NMR (400 MHz, CDCl₃): δ (ppm) 1.80-1.90 (m, 1H), 2.32-2.37 (m, 1H), 2.63-2.68 (m, 1H), 2.84-2.91 (m, 1H), 3.88 (s, 1H), 4.01-4.18 (m, 1H), 7.15-7.25 (m, 4H), 7.30-7.45 (m, 8H).

### Example 241

### Synthesis of 4-hydroxy-1,3-bis(4-methoxyphenyl)-5,5-dimethyl-4-(3-(trifluoromethoxy)phenyl)imidazolidin-2-one

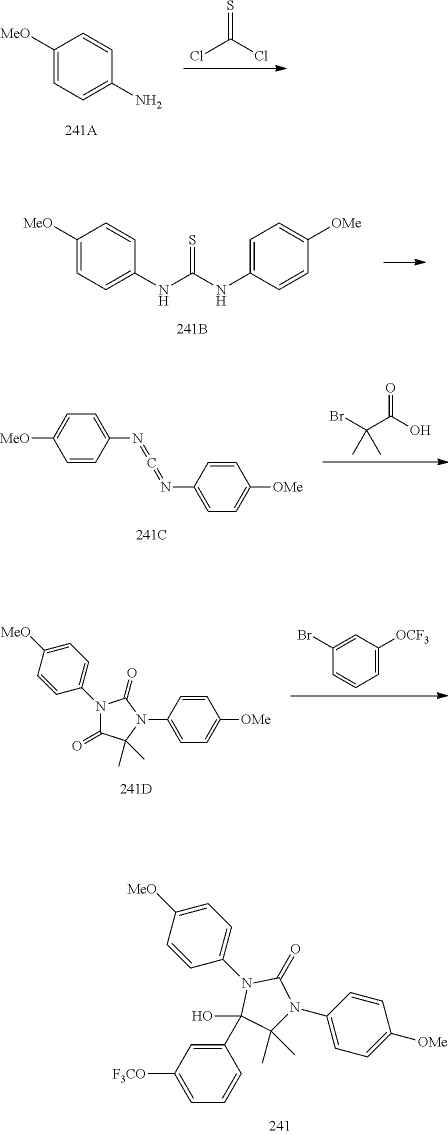

To a solution of 4-methoxyaniline Compound 241A (2.0 g, 16.24 mmol) in dichloromethane (100 mL) was added a saturated aqueous solution of NaHCO₃ (100 mL) at 0° C. To the stilled dichloromethane layer of the two layers mixture was added thiophosgene (1.4 mL, 17.87 mmol). The mixture was stirred vigorously at room temperature for 30 minutes. The organic layer was dried over anhydrous sodium sulfate, filtered, and concentrated under reduced pressure. The concentrate was dissolved in dichloromethane (35 mL) and to it was added a solution of 4-methoxyaniline Compound 241A (1.82 g, 14.77 mmol) in dichloromethane (5 mL). The resulting mixture was stirred at room temperature for 48 hours and concentrated. The concentrated solution was filtered and the solid was washed with cold dichloromethane to afford Compound 241B: LC-MS (ESI) m/z: 289 [M+H]⁺; ¹H-NMR (DMSO-d₆, 400 MHz): δ (ppm) 3.74 (s, 6H), 6.89 (d, J=8.4 Hz, 4H), 7.31 (d, J=8.4 Hz, 4H), 9.44 (s, 2H).

Compounds 241C, 241D, and 241 were synthesized by employing the procedures described for Compounds 120C, 120D, and 125 using Compounds 241B, 241C using K₂CO₃ as base and MeCN as solvent, 241D, and 1-bromo-3-(trifluoromethoxy)benzene in lieu of Compounds 120B, 120C using NaOH as base and dioxane as solvent, 2C, and 3-bromo-N,N-dimethylaniline. Compound 241D. LC-MS (ESI) m/z: 341 [M+H]⁺. Compound 241. LC-MS (ESI) m/z: 503 [M+H]⁺; ¹H-NMR (CD₃OD, 400 MHz): δ (ppm) 0.84 (s, 3H), 1.33 (s, 3H), 3.74 (s, 3H), 3.84 (s, 3H), 6.83 (d, J=8.8 Hz, 2H), 7.02 (d, J=9.2 Hz, 2H), 7.20 (d, J=8.0 Hz, 1H), 7.30 (d, J=8.8 Hz, 2H), 7.38-7.48 (m, 4H), 7.54 (d, J=7.6 Hz, 1H).

### Example 242

### Synthesis of 5,7-bis(4-chlorophenyl)-8-hydroxy-8-(3-(trifluoromethoxy)phenyl)-5,7-diazaspiro[3.4]octan-6-one

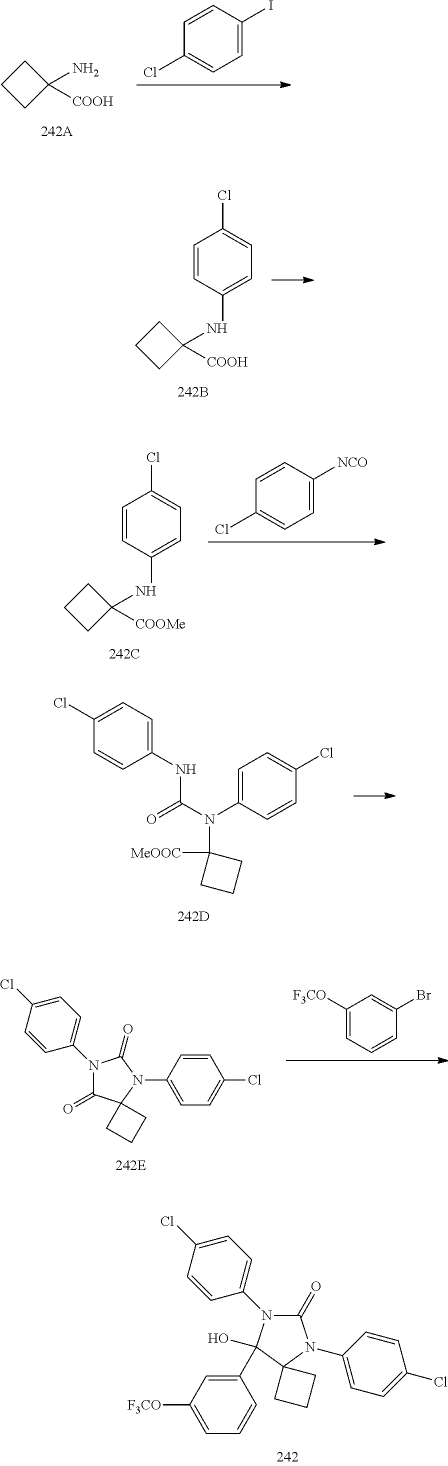

Compounds 242B, 242C, 242D, 242E, and 242 were synthesized by employing the procedures described for Compounds 224B, 224C, 224D, 224E, and 125 using Compounds 242A in the presence of 2-acetylcyclohexan-1-one, 242B, 242C, 242D, 242E, and 1-bromo-3-(trifluoromethoxy)benzene in lieu of Compounds 224A without 2-acetylcyclohexan-1-one, 224B, 224C, 224D, 2C, and 3-bromo-N,N-dimethylaniline. Compound 242B. LC-MS (ESI) m/z: 226 [M+H]⁺. Compound 242C. LC-MS (ESI) m/z: 240 [M+H]⁺; ¹H-NMR (400 MHz, CDCl₃): δ (ppm) 2.02-2.13 (m, 2H), 2.17-2.19 (m, 2H), 2.68-2.74 (m, 2H), 3.68 (s, 3H), 4.29 (s, 1H), 7.35-7.38 (m, 2H), 7.07-7.10 (m, 2H). Compound 242D. LC-MS (ESI) m/z: 393 [M+H]⁺. Compound 242E. LC-MS (ESI) m/z: 361 [M+H]⁺; ¹H-NMR (400 MHz, CDCl₃): δ (ppm) 1.57-1.69 (m, 1H), 2.20-2.23 (m, 1H), 2.41-2.49 (m, 2H), 2.58-2.65 (m, 2H), 7.26-7.30 (m, 2H), 7.43-7.52 (m, 6H). Compound 242. LC-MS (ESI) m/z: 523 [M+H]⁺; ¹H-NMR (400 MHz, CDCl₃): δ (ppm) 0.83-0.94 (m, 1H), 1.08-1.16 (m, 1H), 1.74-1.80 (m, 1H), 1.98-2.06 (m, 1H), 2.22-2.30 (m, 1H), 2.69-2.75 (m, 1H), 3.66 (s, 1H), 7.14-7.26 (m, 5H), 7.33-7.44 (m, 7H).

### Example 243

### Synthesis of 1,3-bis(4-chlorophenyl)-4-propyl-5-(3-(trifluoromethyl)phenyl)-1,3-dihydro-2H-imidazol-2-one

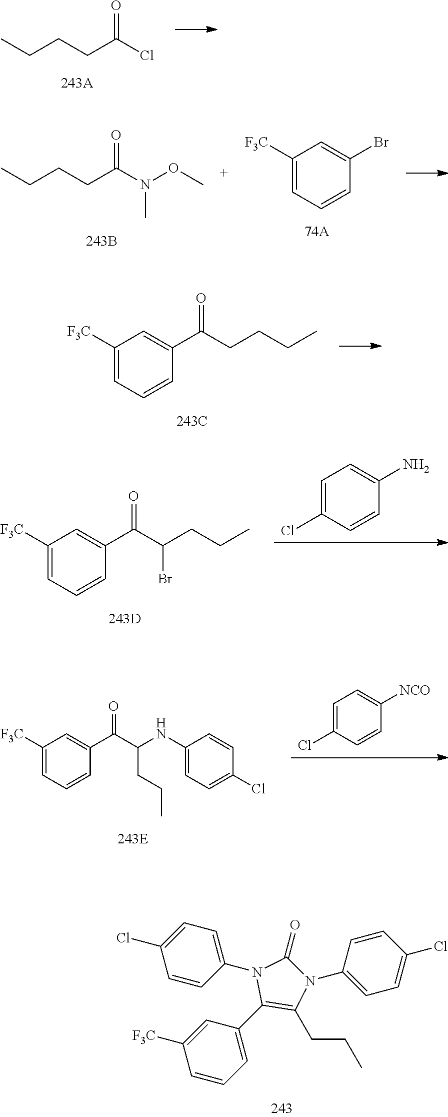

Compounds 243B, 243C, 243D, 243E, and 243 were synthesized by employing the procedures described for Compounds 42B, 59B, 13B, 1B, and 1 using Compounds 243A, 243B, 74A, 243C, 243D, 4-chloroaniline, 243E, and 1-chloro-4-isocyanatobenzene in lieu of Compounds 42A, N-methoxy-N-methylacetamide, 59A, 13A, 1A, 4-bromoaniline, 1B, and 1-bromo-4-isocyanatobenzene. Compound 243B. ¹H-NMR (CDCl₃, 400 MHz): δ (ppm) 0.93 (t, J=7.2 Hz, 3H), 1.34-1.40 (m, 2H), 1.60-1.76 (m, 2H), 2.42 (t, J=7.6 Hz, 2H), 3.18 (s, 3H), 3.68 (s, 3H). Compound 243C. LC-MS (ESI) m/z: 231 [M+H]⁺; ¹H-NMR (CDCl₃, 400 MHz): δ (ppm) 0.97 (t, J=7.2 Hz, 3H), 1.40-1.45 (m, 2H), 1.70-1.78 (m, 2H), 3.00 (t, J=7.2 Hz, 2H), 7.61 (t, J=7.6 Hz, 1H), 7.81 (d, J=7.6 Hz, 1H), 8.14 (d, J=7.6 Hz, 1H), 8.21 (s, 1H). Compound 243D. LC-MS (ESI) m/z: 309 [M+H]⁺. Compound 243E. LC-MS (ESI) m/z: 356 [M+H]⁺; ¹H-NMR (CDCl₃, 400 MHz): δ (ppm) 0.91 (t, J=7.2 Hz, 3H), 1.45-1.52 (m, 2H), 1.81-2.02 (m, 2H), 4.59-4.61 (m, 1H), 4.99-5.04 (m, 1H), 6.60 (d, J=8.8 Hz, 2H), 7.12 (d, J=8.8 Hz, 2H), 7.67 (t, J=8.0 Hz, 1H), 7.88 (d, J=8.0 Hz, 1H), 8.17 (d, J=8.0 Hz, 1H), 8.24 (s, 1H). Compound 243. LC-MS (ESI) m/z: 491 [M+H]⁺; ¹H-NMR (CD₃OD, 400 MHz): δ (ppm) 0.69 (t, J 7.2 Hz, 3H), 1.21-1.26 (m, 2H), 2.52 (t, J 7.2 Hz, 2H), 7.21 (d, J 8.8 Hz, 2H), 7.37 (d, J 8.8 Hz, 2H), 7.45-7.56 (m, 5H), 7.59-7.62 (m, 3H).

### Example 244

### Synthesis of 1,3-bis(4-chlorophenyl)-4-(3-ethylphenyl)-5-propyl-1,3-dihydro-2H-imidazol-2-one

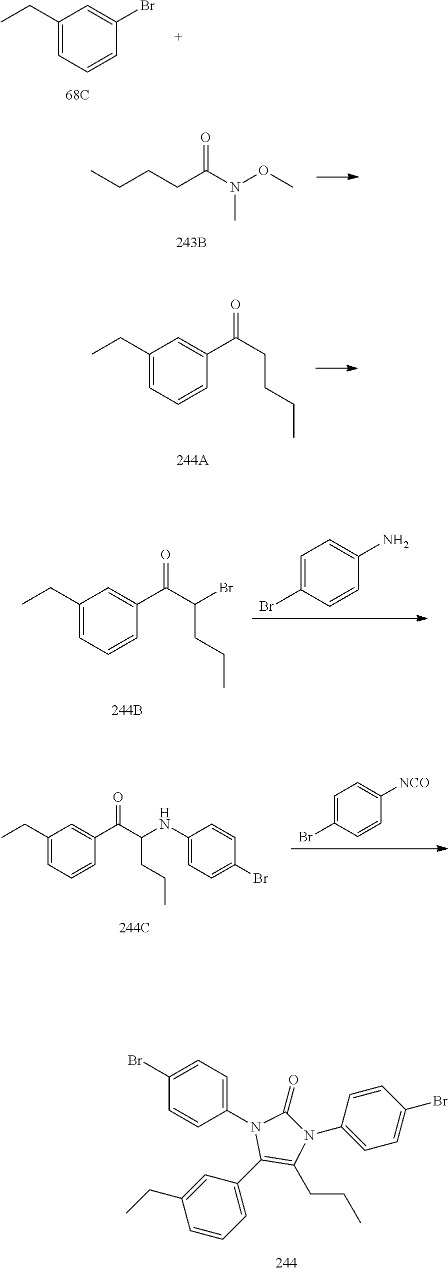

Compounds 244A, 244B, 244C, and 244 were synthesized by employing the procedures described for Compounds 59B, 13B, 1B, and 1 using Compounds 243B, 68C, 244A, 244B, heating at 60° C., and 244C in lieu of N-methoxy-N-methylacetamide, Compounds 59A, 13A, 1A, stirred at room temperature, and 1B. Compound 244A. LC-MS (ESI) m/z: 191 [M+H]⁺. Compound 244B. LC-MS (ESI) m/z: 269 [M+H]⁺; ¹H-NMR (CDCl₃, 400 MHz): δ (ppm) 0.99 (t, J=7.6 Hz, 3H), 1.28 (t, J=8.0 Hz, 3H), 1.43-1.58 (m, 2H), 2.10-2.19 (m, 2H), 2.72 (q, J=7.6 Hz, 2H), 5.16 (t, J=7.6 Hz, 1H), 7.38-7.45 (m, 2H), 7.81-7.85 (m, 2H). Compound 244C. LC-MS (ESI) m/z: 360 [M+H]⁺. Compound 244. LC-MS (ESI) m/z: 451 [M+H]⁺; ¹H-NMR (CDCl₃, 400 MHz): δ (ppm) 0.69 (t, J=7.2 Hz, 3H), 1.10 (t, J=8.0 Hz, 3H), 1.17-1.23 (m, 2H), 2.45 (t, J=7.6 Hz, 2H), 2.55 (q, J=7.6 Hz, 2H), 6.90-6.93 (m, 2H), 7.08-7.12 (m, 3H), 7.18-7.25 (m, 3H), 7.36-7.39 (m, 2H), 7.46-7.49 (m, 2H).

### Example 245

### Synthesis of 1,3-bis(4-chlorophenyl)-4-propyl-5-(3-(trifluoromethoxy)phenyl)-1,3-dihydro-2H-imidazol-2-one

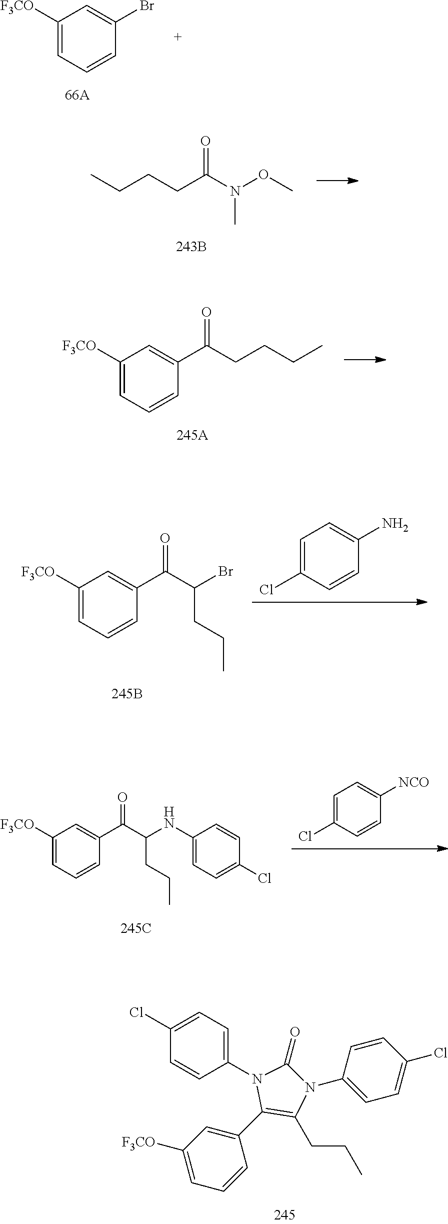

Compounds 245A, 245B, 245C, and 245 were synthesized by employing the procedures described for Compounds 59B, 13B, 1B, and 1 using Compounds 243B, 66A, 245A, 245B, 4-chloroaniline, heating at 60° C., 245C, and 1-chloro-4-isocyanatobenzene in lieu of N-methoxy-N-methylacetamide, Compounds 59A, 13A, 1A, 4-bromoaniline, stirred at room temperature, 1B, and 1-chloro-4-isocyanatobenzene. Compound 245A. LC-MS (ESI) m/z: non-ionizable compound under routine conditions used; ¹H-NMR (CDCl₃, 400 MHz): δ (ppm) 0.96 (t, J=7.6 Hz, 3H), 1.39-1.44 (m, 2H), 1.71-1.75 (m, 2H), 2.96 (t, J=7.2 Hz, 2H), 7.40-7.42 (m, 1H), 7.51 (t, J=8.0 Hz, 1H), 7.80 (s, 1H), 7.89 (d, J=7.6 Hz, 1H). Compound 245B. LC-MS (ESI) m/z: non-ionizable compound under routine conditions used; ¹H-NMR (CDCl₃, 400 MHz): δ (ppm) 1.00 (t, J=7.6 Hz, 3H), 1.43-1.56 (m, 2H), 2.11-2.21 (m, 2H), 5.06-5.09 (m, 1H), 7.44-7.46 (m, 1H), 7.54 (t, J=8.0 Hz, 1H), 7.86 (s, 1H), 7.95 (d, J=7.6 Hz, 1H). Compound 245C. LC-MS (ESI) m/z: 372 [M+H]⁺. Compound 245. LC-MS (ESI) m/z: 507 [M+H]⁺; ¹H-NMR (CDCl₃, 400 MHz): δ (ppm) 0.69 (t, J=7.6 Hz, 3H), 1.18-1.24 (m, 2H), 2.45 (t, J=7.6 Hz, 2H), 6.98-7.03 (m, 2H), 7.09-7.13 (m, 3H), 7.25-7.26 (m, 1H), 7.27-7.50 (m, 6H).

### Example 246

### Synthesis of 4-(3-bromophenyl)-1,3-bis(4-chlorophenyl)-5-methyl-1,3-dihydro-2H-imidazol-2-one

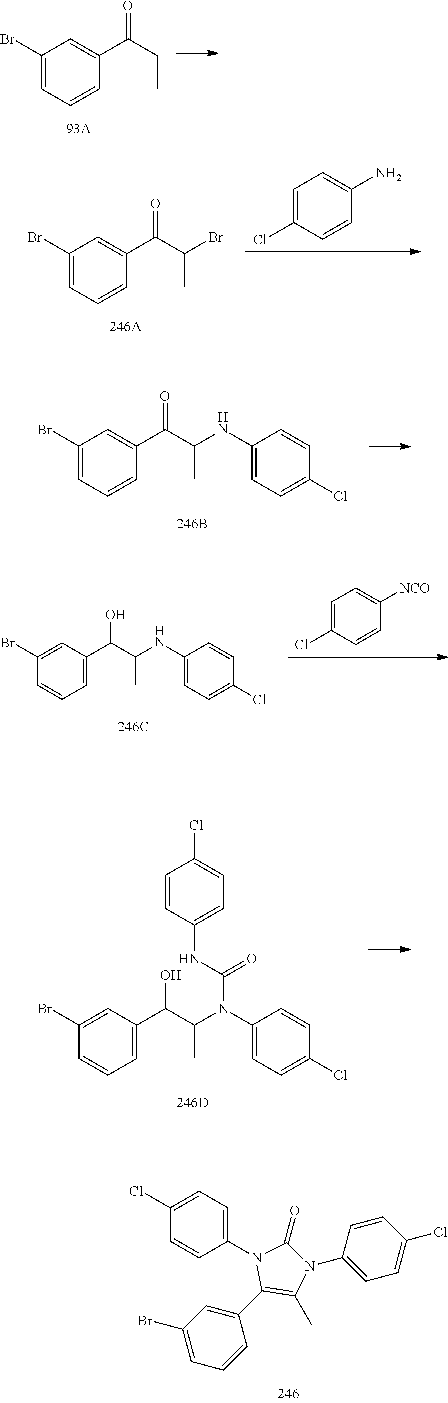

Compounds 246A, 246B, 246C, 246D, and 246 were synthesized by employing the procedures described for Compounds 13B, 13C, 14C, 1, and 14 using Compounds 93A, 246A, 4-chloroaniline, 246B, 246C, 1-chloro-4-isocyanatobenzene, and 246D in lieu of Compounds 13A, 13B, 4-bromoaniline, 14B, 1B, 1-bromo-4-isocyanatobenzene, and 14D. Compound 246A. LC-MS (ESI) m/z: 291 [M+H]⁺. Compound 246B. LC-MS (ESI) m/z: 338 [M+H]⁺. Compound 246C. LC-MS (ESI) m/z: 340 [M+H]⁺. Compound 246D. LC-MS (ESI) m/z: 493 [M+H]⁺. Compound 246. LC-MS (ESI) m/z: 473 [M+H]⁺; ¹H-NMR (CDCl₃, 400 MHz): δ (ppm) 2.06 (s, 3H), 6.91-6.96 (m, 1H), 7.10-7.17 (m, 3H), 7.26-7.30 (m, 2H), 7.32-7.42 (m, 4H), 7.46-7.51 (m, 2H).

### Example 247

### Synthesis of 1,3-bis(4-chlorophenyl)-4-ethyl-5-(3-(trifluoromethoxy)phenyl)-1,3-dihydro-2H-imidazol-2-one

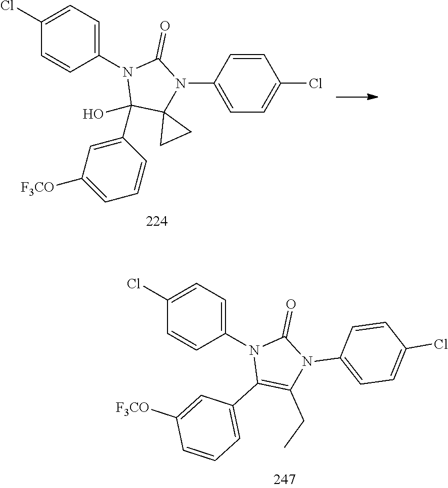 [SMILES: O=C1N(c2ccc(Cl)cc2)C2(CC2)C(O)(c2cccc(OC(F)(F)F)c2)N1c1ccc(Cl)cc1 ; CCc1c(-c2cccc(OC(F)(F)F)c2)n(-c2ccc(Cl)cc2)c(=O)n1-c1ccc(Cl)cc1]

Compound 247 was synthesized by employing the procedure described for Compound 255 using Compound 224 in lieu of Compound 1. LC-MS (ESI) m/z: 493 [M+H]⁺. ¹H-NMR (400 MHz, CDCl₃): δ (ppm) 0.87 (t, J=7.2 Hz, 3H), 2.50 (q, J=7.2 Hz, 2H), 6.96-7.11 (m, 5H), 7.25 (m, 1H), 7.27 (m, 1H), 7.31-7.39 (m, 3H), 7.48-7.50 (m, 2H).

### Example 248

### Synthesis of 1,3-bis(4-bromophenyl)-4-propyl-5-(3-(trifluoromethoxy)phenyl)-1,3-dihydro-2H-imidazol-2-one

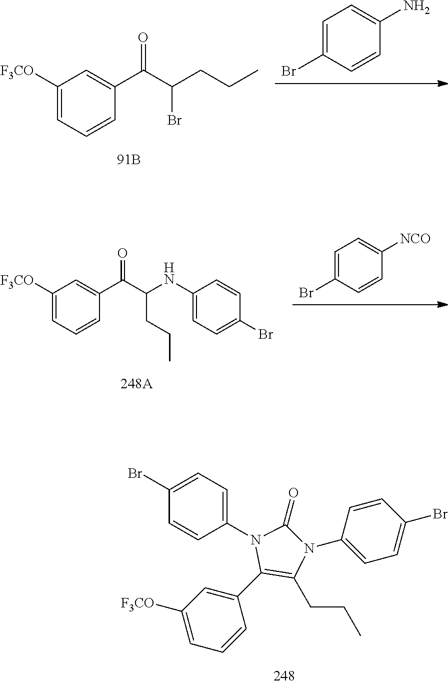

Compounds 248A and 248 were synthesized by employing the procedures described for Compounds 1B and 1 using Compounds 91B, heating at 60° C., and 248A in lieu of Compounds 1A, stirred at room temperature, and 1B. Compound 248A. LC-MS (ESI) m/z: 416 [M+H]⁺. Compound 248. LC-MS (ESI) m/z: 595 [M+H]⁺; ¹H-NMR (CDCl₃, 400 MHz): δ (ppm) 0.69 (t, J=7.6 Hz, 3H), 1.20-1.22 (m, 2H), 2.45 (t, J=7.6 Hz, 2H), 6.99-7.05 (m, 4H), 7.12-7.13 (m, 1H), 7.30-7.33 (m, 3H), 7.39-7.42 (m, 2H), 7.63-7.65 (m, 2H).

### Example 249

### Synthesis of 1,3-bis(4-bromophenyl)-4-cyclopropyl-5-phenyl-1,3-dihydro-2H-imidazol-2-one

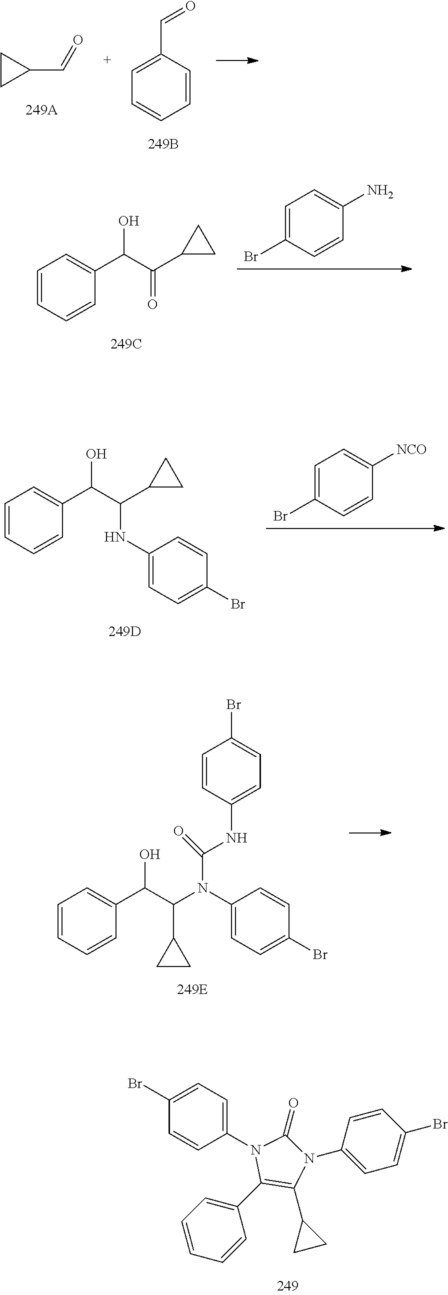

To a mixture of Compound 249A (17.8 mmol) and sodium cyanide (1.75 mg, 35.6 mmol) in ethanol (40 mL) and H₂O (7 mL) was added benzaldehyde 249B (1.89 g, 17.8 mmol). The mixture was stirred at 100° C. for 3 hours, cooled down to room temperature, diluted with water (100 mL), and extracted with ethyl acetate (60 mL×3). The combined organic layers was washed with water (50 mL×2) and brine (50 mL), dried over anhydrous sodium sulfate, filtered, and concentrated to give a crude product, which was purified with flash column chromatography on silica gel (ethyl acetate in petroleum ether, 20% v/v) to afford Compound 249C. LC-MS (ESI) m/z: 177 [M+H]⁺; ¹H-NMR (CDCl₃, 400 MHz): δ (ppm) 0.78-0.83 (m, 1H), 1.00-1.16 (m, 2H), 1.24-1.28 (m, 1H), 1.85-1.89 (m, 1H), 4.40 (d, J=4.4 Hz, 1H), 5.26 (d, J=4.4 Hz, 1H), 7.34-7.39 (m, 5H).

A solution of Compound 249C (1.0 g, 5.7 mmol) and 4-bromoaniline (1.0 g, 5.7 mmol) in ethanol (10 mL) was stirred at 130° C. for 16 hours in a sealed vial. After the reaction mixture was cooled down to room temperature, to it was added NaBH₄ (0.2 g, 5.7 mmol), stirred at 25° C. for 0.5 hour, and evaporated under reduced pressure. The residue was purified with preparative HPLC to afford Compound 249D. LC-MS (ESI) m/z: 332 [M+H]⁺; ¹H-NMR (CDCl₃, 400 MHz): δ (ppm) −0.06-0.00 (m, 1H), 0.14-0.18 (m, 1H), 0.34-0.40 (m, 2H), 0.82-0.84 (m, 1H), 2.54 (s, 1H), 3.02 (s, 1H), 3.79 (s, 1H), 5.01 (s, 1H), 6.58 (d, J=8.8 Hz, 2H), 7.25 (d, J=8.8 Hz, 2H), 7.30-7.40 (m, 5H).

Compounds 249E and 249 were synthesized by employing the procedures described for Compound 1 and 14 using Compound 249D and 249E in lieu of Compound 1B and 14D. Compound 249E. LC-MS (ESI) m/z: 529 [M+H]⁺; ¹H-NMR (CD₃OD, 400 MHz): δ (ppm) −0.05-−0.02 (m, 1H), 0.20-0.23 (m, 1H), 0.31-0.34 (m, 1H), 0.47-0.49 (m, 1H), 0.99 (s, 1H), 3.66 (d, J=6.8 Hz, 1H), 5.19 (d, J=4.4 Hz, 1H), 7.15-7.37 (m, 10H), 7.45-7.47 (m, 2H), 7.53-7.55 (m, 2H). Compound 249. LC-MS (ESI) m/z: 509 [M+H]⁺; ¹H-NMR (CD₃OD, 400 MHz): δ (ppm) −0.01-0.01 (m, 2H), 0.48-0.52 (m, 2H), 1.71-1.76 (m, 1H), 7.06-7.08 (m, 2H), 7.15-7.18 (m, 2H), 7.25-7.27 (m, 3H), 7.43-7.47 (m, 4H), 7.65-7.68 (m, 2H).

### Example 250

### Synthesis of 1,3-bis(4-bromophenyl)-4-(3-chlorophenyl)-5-propyl-1,3-dihydro-2H-imidazol-2-one

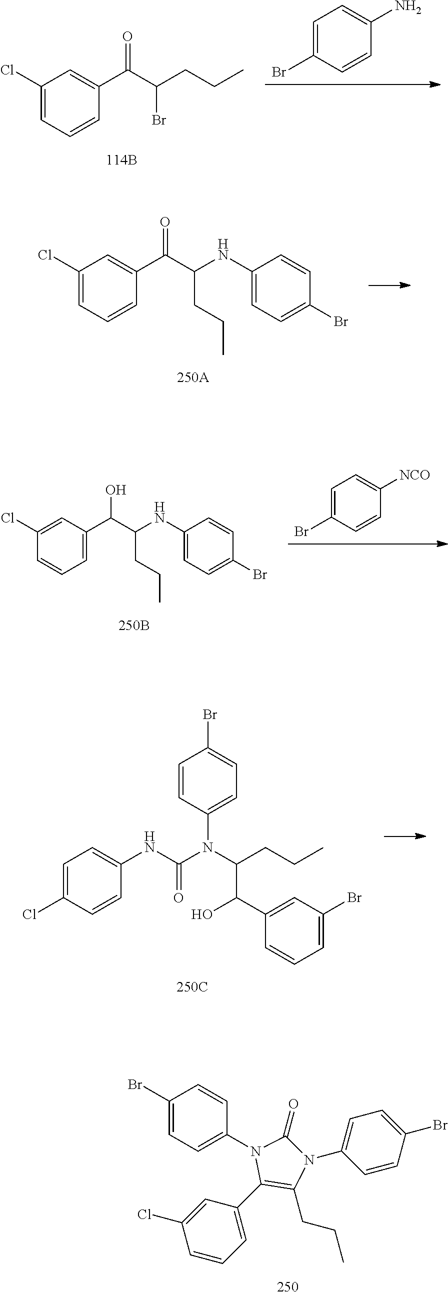

Compounds 250A, 250B, 250C, and 250 were synthesized by employing the procedures described for Compounds 1B, 14C, and 14 using 114B, 250A, 250B, and 250C in lieu of 1A, 14B, 1B, and 14D. Compound 250A. LC-MS (ESI) m/z: 366 [M+H]⁺. Compound 250B. LC-MS (ESI) m/z: 368 [M+H]⁺. Compound 250C. LC-MS (ESI) m/z: 565 [M+H]⁺. Compound 250. LC-MS (ESI) m/z: 545 [M+H]⁺; ¹H-NMR (CDCl₃, 400 MHz): δ (ppm) 0.68 (t, J=7.2 Hz, 3H), 1.43-1.16 (m, 2H), 2.44 (t, J=8.0 Hz, 2H), 6.89 (d, J=14.0 Hz, 1H), 7.03 (d, J=8.8 Hz, 2H), 7.17-7.31 (m, 5H), 7.40 (d, J=8.8 Hz, 2H), 7.62 (d, J=9.2 Hz, 2H).

### Example 251

### Synthesis of 3-(5,7-bis(4-chlorophenyl)-8-hydroxy-6-oxo-2-oxa-5,7-diazaspiro[3.4]octan-8-yl)benzonitrile

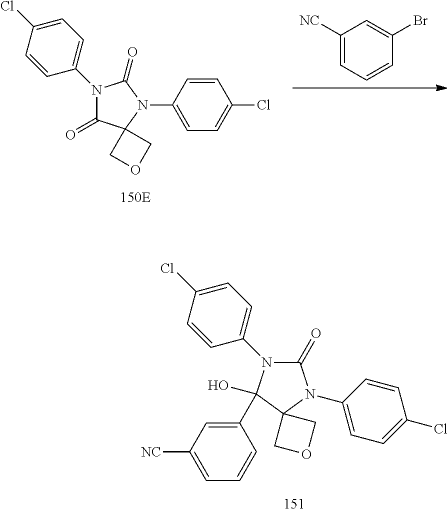 [SMILES: O=C1N(c2ccc(Cl)cc2)C(=O)C2(COC2)N1c1ccc(Cl)cc1 ; N#Cc1cccc(Br)c1 ; N#Cc1cccc(C2(O)N(c3ccc(Cl)cc3)C(=O)N(c3ccc(Cl)cc3)C23COC3)c1]

Compound 151 was synthesized by employing the procedure described for Compound 125 using Compound 150E and 3-bromobenzonitrile in lieu of Compound 2C and 3-bromo-N,N-dimethylaniline. LC-MS (ESI) m/z: 466 [M+H]⁺; ¹H-NMR (CDCl₃, 400 MHz): δ (ppm) 3.87 (d, J=7.6 Hz, 1H), 4.41 (d, J=8.4 Hz, 1H), 4.63 (d, J=8.4 Hz, 1H), 5.05 (s, 1H), 5.12 (d, J=8.4 Hz, 1H), 7.14-7.20 (m, 6H), 7.37 (d, J=8.8 Hz, 2H), 7.45-7.55 (m, 2H), 7.64 (d, J=8.4 Hz, 1H), 7.84 (s, 1H).

### Example 252

### Synthesis of 3-(5-ethyl-1,3-bis(4-fluorophenyl)-2-oxo-2,3-dihydro-1H-imidazol-4-yl)benzonitrile

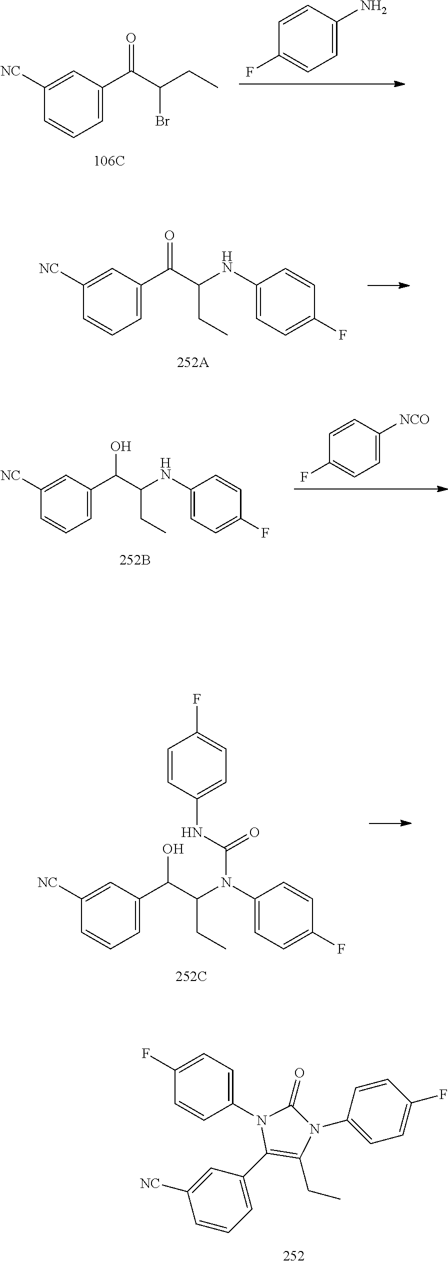

Compounds 252A, 252B, and 252C were synthesized by employing the procedures described for Compounds 1B, 14C, 1, and 14 using 106C, 4-fluoroaniline, using NMP as solvent, Compounds 252A, 252B, 1-floro-4-isocyanatobenzene, and 252C in lieu of 1A, 4-bromoaniline, using EtOH as solvent, 14B, 1B, 1-bromo-4-isocyanatobenzene, and 14D. Compound 252A. LC-MS (ESI) m/z: 283 [M+H]⁺; ¹H-NMR (CDCl₃, 400 MHz): δ (ppm) 0.94 (t, J=7.2 Hz, 3H), 1.71-1.73 (m, 1H), 2.01-2.05 (m, 1H), 4.89-4.92 (m, 1H), 6.60-6.64 (m, 2H), 6.88-6.91 (m, 2H), 7.63-7.67 (m, 1H), 7.87-7.89 (m, 1H), 8.19-8.21 (m, 1H), 8.26 (s, 1H). Compound 252B. LC-MS (ESI) m/z: 285 [M+H]⁺; ¹H-NMR (CDCl₃, 400 MHz): δ (ppm) 0.89 (t, J=7.2 Hz, 3H), 1.27-1.39 (m, 1H), 1.60-1.65 (m, 1H), 2.58-2.60 (m, 1H), 3.10-3.45 (m, 2H), 4.95 (s, 1H), 6.57-6.66 (m, 2H), 6.85-6.93 (m, 2H), 7.45-7.49 (m, 1H), 7.56-7.63 (m, 2H), 7.69-7.70 (m, 1H). Compound 252C. LC-MS (ESI) m/z: 422 [M+H]⁺.

The crude product was purified with chiral HPLC to give Compound 252. LC-MS (ESI) m/z: 402 [M+H]⁺; ¹H-NMR (CD₃OD, 400 MHz): δ (ppm) 0.76 (t, J=8.0 Hz, 3H), 2.42 (q, J=8.0 Hz, 2H), 6.99-7.03 (m, 2H), 7.13-7.16 (m, 2H), 7.20-7.24 (m, 2H), 7.38-7.44 (m, 4H), 7.50-7.56 (m, 2H).

### Example 253

### Synthesis of 4-(3-acetylphenyl)-1,3-bis(4-chlorophenyl)-5-methyl-1,3-dihydro-2H-imidazol-2-one

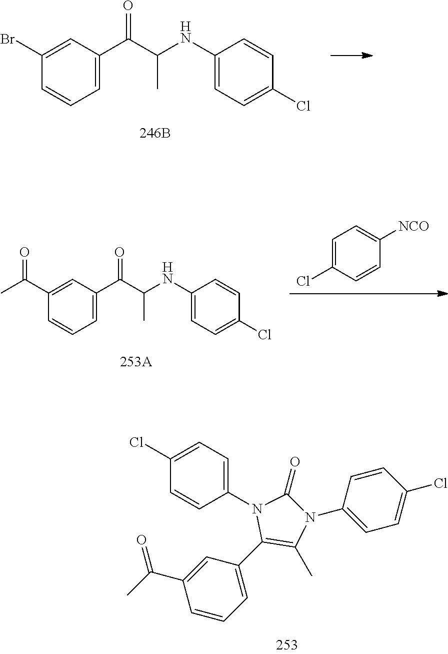

Compounds 253A and 253 were synthesized by employing the procedures described for Compounds 47B and 1 using Compounds 246B, 253A, and 1-chloro-4-isocyanatobenzene in lieu of Compounds 47A, 1B and 1-bromo-4-isocyanatobenzene. Compound 253A. LC-MS (ESI) m/z: 302 [M+H]⁺. Compound 253. LC-MS (ESI) m/z: 437 [M+H]⁺; ¹H-NMR (CDCl₃, 400 MHz): δ (ppm) 2.00 (s, 3H), 2.44 (s, 3H), 7.03-7.08 (m, 2H), 7.14-7.22 (m, 3H), 7.28-7.35 (m, 3H), 7.39-7.45 (m, 2H), 7.65-7.80 (m, 2H).

### Example 254

### Synthesis of 4-(3-chlorophenyl)-1,3-bis(4-chlorophenyl)-5-(tetrahydrofuran-2-yl)-1,3-dihydro-2H-imidazol-2-one

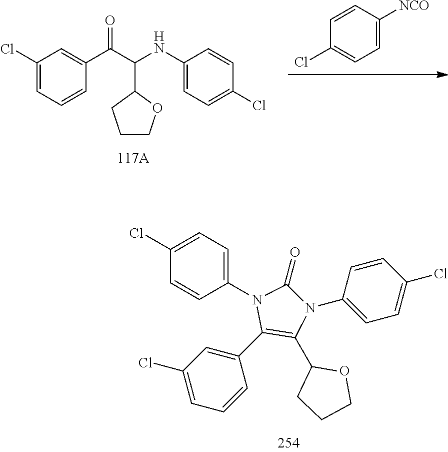

Compound 254 was synthesized by employing the procedure described for Compound 86A using Compound 117A and 1-chloro-4-isocyanatobenzene in lieu of Compound 1B and 1-bromo-4-isocyanatobenzene. LC-MS (ESI) m/z: 485 [M+H]⁺; ¹H-NMR (CDCl₃, 400 MHz): δ (ppm) 1.41-1.59 (m, 2H), 1.70-1.81 (m, 2H), 3.53-3.66 (m, 2H), 4.63 (q, J=7.2 Hz, 1H), 6.99-7.12 (m, 3H), 7.21-7.24 (m, 2H), 7.28-7.31 (m, 3H), 7.44-7.51 (m, 4H).

### Example 255

### Synthesis of 1,3-bis(4-bromophenyl)-4-phenyl-1,3-dihydro-2H-imidazol-2-one

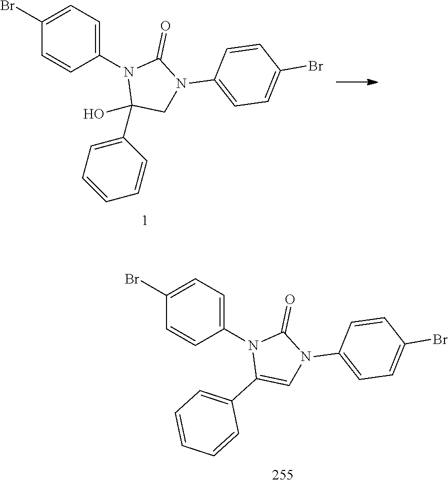 [SMILES: O=C1N(c2ccc(Br)cc2)CC(O)(c2ccccc2)N1c1ccc(Br)cc1 ; O=c1n(-c2ccc(Br)cc2)cc(-c2ccccc2)n1-c1ccc(Br)cc1]

To a solution of Compound 1 (20 mg, 0.041 mmol) in dichloromethane (5 mL) was added TFA (3 drops). The mixture was stirred at room temperature for 2 hours. The reaction mixture was concentrated and the residue was purified by preparative HPLC to give Compound 255. LC-MS (ESI) m/z: 469 [M+H]⁺; ¹H-NMR (DMSO-d₆, 400 MHz): δ (ppm) 7.15-7.17 (m, 2H), 7.20-7.22 (m, 2H), 7.30-7.32 (m, 3H), 7.61-7.64 (m, 3H), 7.68-7.71 (m, 2H), 7.82-7.84 (m, 2H).

### Example 256

### Synthesis of 1,3-bis(4-bromophenyl)-4-(1H-pyrrol-2-yl)-1,3-dihydro-2H-imidazol-2-one

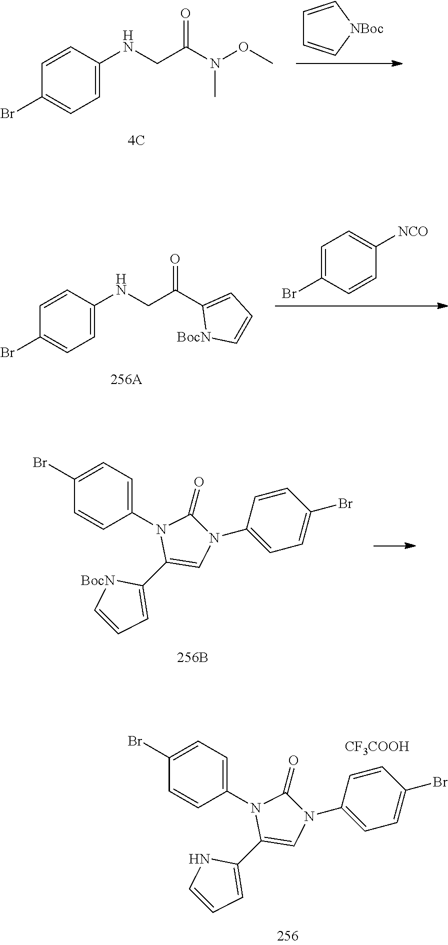

To a solution of 2,2,6,6-tetramethylpiperidine (1.69 g, 12 mmol) in dry THF (10 mL) was dropped a solution of n-BuLi in THF (2.5 M, 4.8 mL, 12 mmol) at −68° C. After addition the mixture was stirred at −68° C. for 5 minutes, warmed to −10° C. for 5 minutes, and then cooled down to −68° C. To the mixture was added a solution of tert-butyl 1H-pyrrole-1-carboxylate (2 g, 12 mmol) in THF (10 mL) and stirred at −68° C. for 1 hours. A solution of Compound 4C (1 g, 4 mmol) in dry THF (5 mL) was added. The mixture was stirred at −68° C. for 20 minutes, quenched with water (10 mL), and extracted with ethyl acetate (20 mL×3). The combined organic layers was washed with brine (40 mL), dried over anhydrous sodium sulfate, filtered, and concentrated under reduced pressure. The residue was purified with flash column chromatography on silica gel (ethyl acetate in petroleum ether, from 0% to 20% v/v) to yield Compound 256A. LC-MS (ESI) m/z: 379 [M+H]⁺; ¹H-NMR (CDCl₃, 400 MHz): δ (ppm) 1.56 (s, 9H), 4.33 (d, J=5.2 Hz, 2H), 4.78 (s, 1H), 6.23 (t, J=2.8 Hz, 1H), 6.53 (d, J=8.8 Hz, 2H), 6.94-6.95 (m, 1H), 7.27 (d, J=8.8 Hz, 2H), 7.41-7.42 (m, 1H).

Compounds 256B and 256 were synthesized by employing the procedures described for Compounds 12 and 204 using Compounds 256A, 1-bromo-4-isocyanatobenzene, and 256B in lieu of Compounds 12B, 1-chloro-4-isocyanatobenzene, and 204. Compound 256B. LC-MS: (ESI) m/z: 558 [M+H]⁺. Compound 256. LC-MS (ESI) m/z: 458 [M+H]⁺; ¹H-NMR (CD₃OD, 400 MHz): δ (ppm) 5.60 (d, J=5.2 Hz, 1H), 6.00 (t, J=2.8 Hz, 1H), 6.73 (s, 1H), 7.07 (s, 1H), 7.25 (d, J=8.8 Hz, 2H), 7.60-7.66 (m, 6H).

### Example 257

### Synthesis of 1,3-bis(4-bromophenyl)-4-methyl-5-phenyl-1,3-dihydro-2H-imidazol-2-one

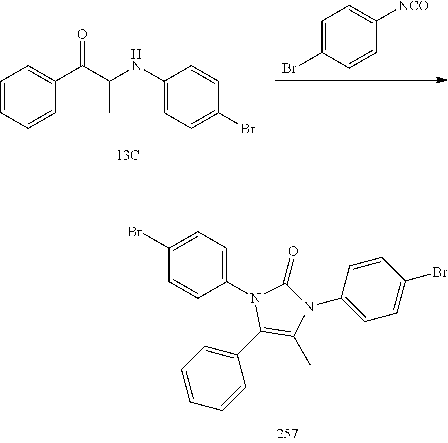

Compound 257 was synthesized by employing the procedure described for Compound 12 using Compound 13C and 1-bromo-4-isocyanatobenzene in lieu of Compound 12B and 1-chloro-4-isocyanatobenzene. LC-MS: (ESI) m/z: 483 [M+H]⁺; ¹H-NMR (CDCl₃, 400 MHz): δ (ppm) 2.04 (s, 3H), 7.04-7.10 (m, 4H), 7.26-7.33 (m, 5H), 7.39 (d, J=8.4 Hz, 2H), 7.62 (d, J=8.4 Hz, 2H).

### Example 258

### Synthesis of 3-(1,3-bis(4-chlorophenyl)-5-ethyl-2-oxo-2,3-dihydro-1H-imidazol-4-yl)benzonitrile

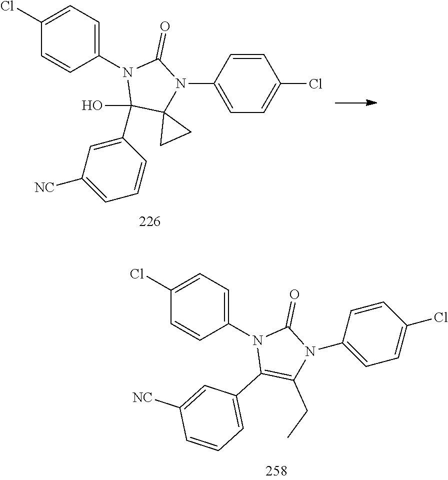 [SMILES: N#Cc1cccc(C2(O)N(c3ccc(Cl)cc3)C(=O)N(c3ccc(Cl)cc3)C23CC3)c1 ; CCc1c(-c2cccc(C#N)c2)n(-c2ccc(Cl)cc2)c(=O)n1-c1ccc(Cl)cc1]

Compound 258 was synthesized by employing the procedure described for Compound 273 using Compound 226 in lieu of Compound 225. LC-MS (ESI) m/z: 434 [M+H]⁺; ¹H-NMR (CDCl₃, 400 MHz): δ (ppm) 0.86 (t, J=8 Hz, 3H), 2.48 (q, J=8 Hz, 2H), 7.08-7.10 (m, 2H), 7.26-7.29 (m, 3H), 7.36-7.58 (m, 7H).

### Example 259

### Synthesis of 1,3-bis(4-chlorophenyl)-4-(m-tolyl)-1,3-dihydro-2H-imidazol-2-one

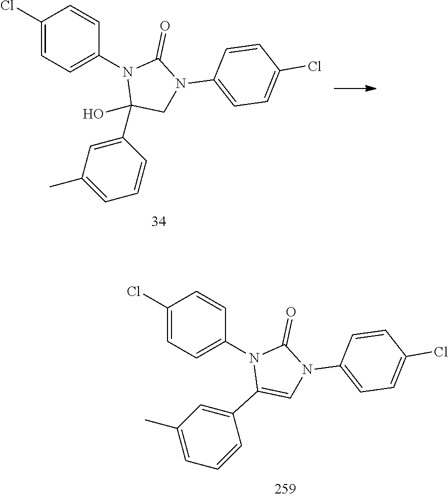 [SMILES: Cc1cccc(C2(O)CN(c3ccc(Cl)cc3)C(=O)N2c2ccc(Cl)cc2)c1 ; Cc1cccc(-c2cn(-c3ccc(Cl)cc3)c(=O)n2-c2ccc(Cl)cc2)c1]

Compound 259 was synthesized by employing the procedure described for Compound 255 using Compound 34 lieu of Compound 1. LC-MS (ESI) m/z: 395 [M+H]⁺; ¹H-NMR (CDCl₃, 400 MHz): δ (ppm) 2.29 (s, 3H), 6.76 (s, 1H), 6.82 (d, J=7.2 Hz, 1H), 6.99 (s, 1H), 7.08-7.20 (m, 4H), 7.33 (d, J=8.0 Hz, 2H), 7.42 (d, J=8.8 Hz, 2H), 7.66 (d, J=8.8 Hz, 2H).

### Example 260

### Synthesis of 1,3-bis(4-chlorophenyl)-4-methyl-5-(m-tolyl)-1,3-dihydro-2H-imidazol-2-one

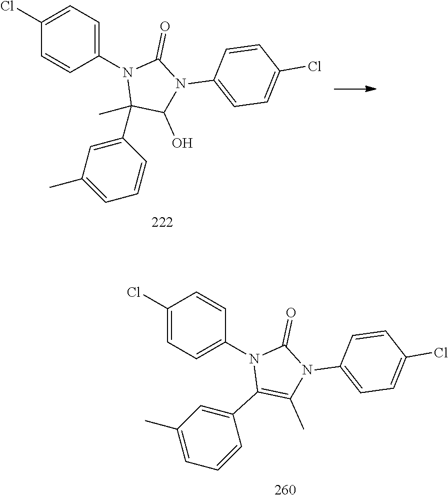 [SMILES: Cc1cccc(C2(C)C(O)N(c3ccc(Cl)cc3)C(=O)N2c2ccc(Cl)cc2)c1 ; Cc1cccc(-c2c(C)n(-c3ccc(Cl)cc3)c(=O)n2-c2ccc(Cl)cc2)c1]

To a solution of Compound 222 (100 mg, 0.23 mmol) in methanol (20 mL) was added p-TsOH (4 mg, 0.23 mmol) and heated at reflux for 2 hours. The reaction mixture was concentrated under reduced pressure. The residue was purified with preparative HPLC to yield Compound 260. LC-MS: (ESI) m/z: 409 [M+H]⁺; ¹H-NMR (DMSO-d₆, 400 MHz): δ (ppm) 1.99 (s, 3H), 2.24 (s, 3H), 6.86 (d, J=7.2 Hz, 1H), 7.03 (s, 1H), 7.09 (d, J=7.6 Hz, 1H), 7.16-7.22 (m, 3H), 7.41 (d, J=8.4 Hz, 2H), 7.53 (d, J=8.8 Hz, 2H), 7.60 (d, J=8.8 Hz, 2H).

### Example 261

### Synthesis of 1,3-bis(4-bromophenyl)-4-phenyl-5-propyl-1,3-dihydro-2H-imidazol-2-one

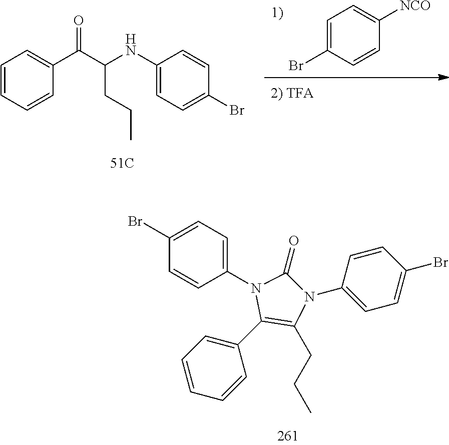

Compound 261 was synthesized by employing the procedure described for Compound 86A using Compound 51C lieu of Compound 1B. LC-MS (ESI) m/z: 511 [M+H]⁺; ¹H-NMR (CD₃OD, 400 MHz): δ (ppm) 0.67 (t, J 7.2 Hz, 3H), 1.15-1.25 (m, 2H), 2.51 (t, J 7.2 Hz, 2H), 7.11 (d, J 8.8 Hz, 2H), 7.20 (dd, J 7.6, 2.0 Hz, 2H), 7.31-7.35 (m, 3H), 7.42-7.48 (m, 4H), 7.74 (d, J 9.2 Hz, 2H).

### Example 262

### Synthesis of 1,3-bis(4-bromophenyl)-4-methyl-5-(3-(trifluoromethoxy)phenyl)-1,3-dihydro-2H-imidazol-2-one

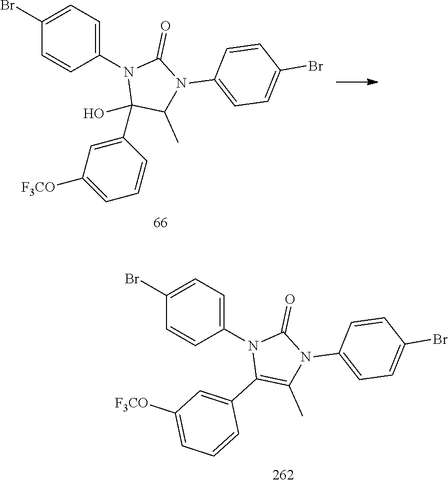 [SMILES: CC1N(c2ccc(Br)cc2)C(=O)N(c2ccc(Br)cc2)C1(O)c1cccc(OC(F)(F)F)c1 ; Cc1c(-c2cccc(OC(F)(F)F)c2)n(-c2ccc(Br)cc2)c(=O)n1-c1ccc(Br)cc1]

Compound 262 was synthesized by employing the procedure described for Compound 255 using Compound 66 lieu of Compound 1. LC-MS (ESI) m/z: 567 [M+H]⁺; ¹H-NMR (CD₃OD, 400 MHz): δ (ppm) 2.08 (s, 3H), 7.03 (s, 1H), 7.13-7.15 (m, 2H), 7.22-7.24 (m, 2H), 7.42-7.44 (m, 3H), 7.51-7.54 (m, 2H), 7.72-7.74 (m, 2H).

### Example 263

### Synthesis of 1,3-bis(4-bromophenyl)-4-methyl-5-(m-tolyl)-1,3-dihydro-2H-imidazol-2-one

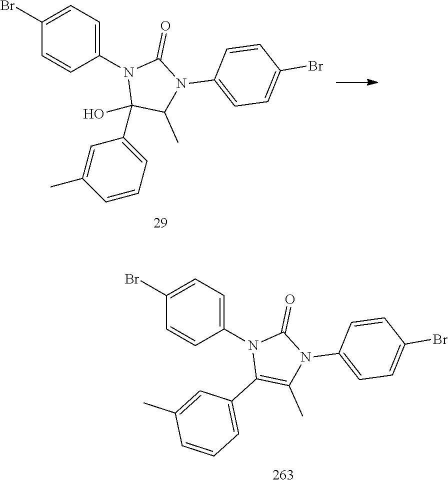 [SMILES: Cc1cccc(C2(O)C(C)N(c3ccc(Br)cc3)C(=O)N2c2ccc(Br)cc2)c1 ; Cc1cccc(-c2c(C)n(-c3ccc(Br)cc3)c(=O)n2-c2ccc(Br)cc2)c1]

Compound 263 was synthesized by employing the procedure described for Compound 255 using Compound 29 lieu of Compound 1. LC-MS (ESI) m/z: 497 [M+H]⁺; ¹H-NMR: (DMSO-d₆, 400 MHz): δ (ppm) 1.99 (s, 3H), 2.24 (s, 3H), 6.84-6.85 (m, 1H), 7.02-7.07 (m, 1H), 7.09-7.11 (m, 3H), 7.17-7.21 (m, 1H), 7.45-7.47 (m, 2H), 7.52-7.54 (m, 2H), 7.72-7.74 (m, 2H).

### Example 264

### Synthesis of 1,3-bis(4-bromophenyl)-4-ethyl-5-phenyl-1,3-dihydro-2H-imidazol-2-one

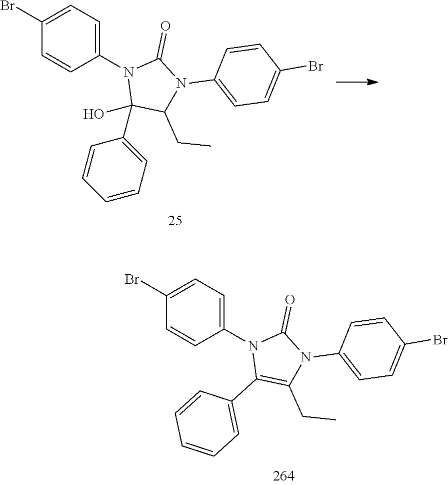 [SMILES: CCC1N(c2ccc(Br)cc2)C(=O)N(c2ccc(Br)cc2)C1(O)c1ccccc1 ; CCc1c(-c2ccccc2)n(-c2ccc(Br)cc2)c(=O)n1-c1ccc(Br)cc1]

Compound 264 was synthesized by employing the procedure described for Compound 255 using Compound 25 lieu of Compound 1. LC-MS (ESI) m/z: 497 [M+H]⁺; ¹H-NMR: (DMSO-d₆, 400 MHz): δ (ppm) 0.74 (t, J=7.2 Hz, 3H), 2.42-2.48 (m, 2H), 7.08-7.07 (m, 4H), 7.28-7.36 (m, 3H), 7.46-7.52 (m, 4H), 7.73-7.75 (m, 2H).

### Example 265

### Synthesis of 3-(1,3-bis(4-chlorophenyl)-2-oxo-2,3-dihydro-1H-imidazol-4-yl)-5-chlorobenzonitrile

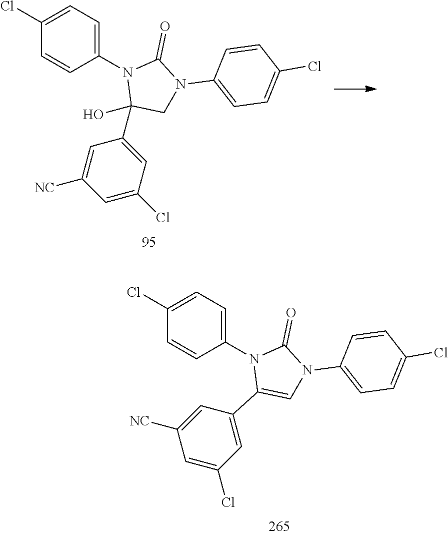 [SMILES: N#Cc1cc(Cl)cc(C2(O)CN(c3ccc(Cl)cc3)C(=O)N2c2ccc(Cl)cc2)c1 ; N#Cc1cc(Cl)cc(-c2cn(-c3ccc(Cl)cc3)c(=O)n2-c2ccc(Cl)cc2)c1]

Compound 265 was synthesized by employing the procedure described for Compound 255 using Compound 95 lieu of Compound 1. LC-MS (ESI) m/z: 440 [M+H]⁺; ¹H-NMR (CDCl₃, 400 MHz): δ (ppm) 6.92 (s, 1H), 7.17 (d, J=8.8 Hz, 2H), 7.26 (s, 1H), 7.31-7.32 (m, 1H), 7.40-7.47 (m, 4H), 7.52-7.53 (m, 1H), 7.61 (d, J=8.8 Hz, 2H).

### Example 266

### Synthesis of 1,3-bis(4-bromophenyl)-4-methyl-5-(4-methylthiophen-2-yl)-1,3-dihydro-2H-imidazol-2-one

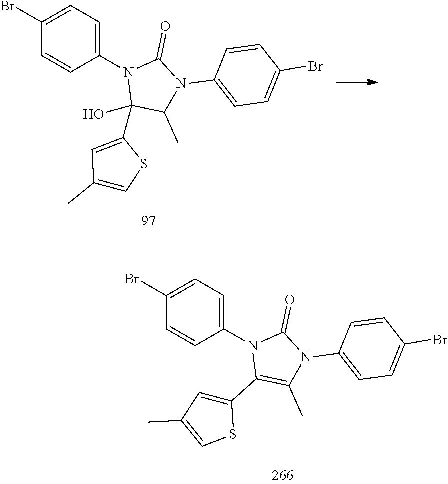 [SMILES: Cc1csc(C2(O)C(C)N(c3ccc(Br)cc3)C(=O)N2c2ccc(Br)cc2)c1 ; Cc1csc(-c2c(C)n(-c3ccc(Br)cc3)c(=O)n2-c2ccc(Br)cc2)c1]

Compound 266 was synthesized by employing the procedure described for Compound 255 using Compound 97 lieu of Compound 1. LC-MS (ESI) m/z: 503 [M+H]⁺; ¹H-NMR (CDCl₃, 400 MHz): δ (ppm) 2.10 (s, 3H), 2.21 (s, 3H), 6.60 (s, 1H), 6.87 (s, 1H), 7.15 (d, J 8.8 Hz, 2H), 7.31 (d, J 8.8 Hz, 2H), 7.47 (d, J 8.8 Hz, 2H), 7.63 (d, J 8.8 Hz, 2H).

### Example 267

### Synthesis of 1,3-bis(4-bromophenyl)-4-isopropyl-5-phenyl-1,3-dihydro-2H-imidazol-2-one

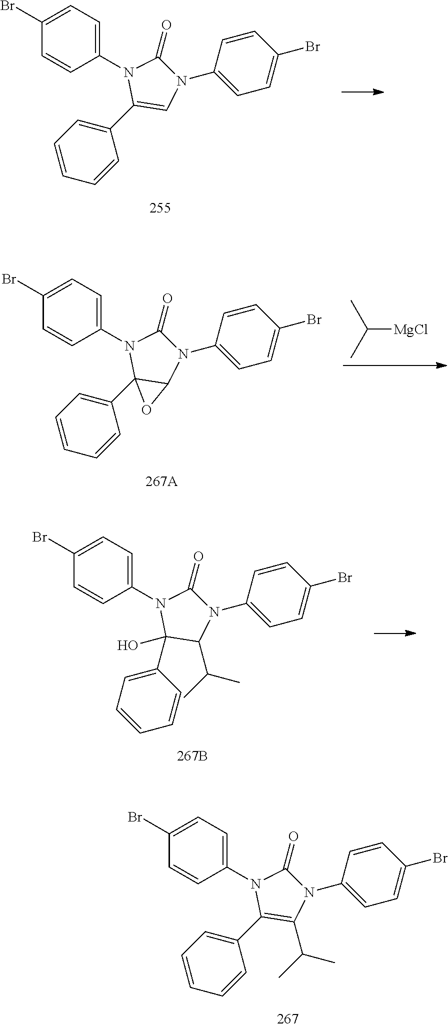

Compounds 267A, 267B, and 267 were synthesized by employing the procedures described for Compounds 86B, 86, and 255 using Compound 255, 267A, isopropylmagnesium chloride, and Compounds 267B in lieu of Compounds 86A, 86B, phenylmagnesium bromide, and Compound 1. Compound 267A. LC-MS (ESI) m/z: 991 [2M+Na]⁺; ¹H-NMR (CDCl₃, 400 MHz): δ (ppm) 5.21-5.48 (m, 1H), 7.18-7.33 (m, 9H), 7.40-7.81 (m, 4H). Compound 267B. LC-MS (ESI) m/z: 529 [M+H]⁺. Compound 267. LC-MS (ESI) m/z: 511 [M+H]⁺; ¹H-NMR (DMSO-d₆, 400 MHz): δ (ppm) 0.94 (d, J=7.6 Hz, 6H), 2.68-2.76 (m, 1H), 7.08 (d, J=8.8 Hz, 2H), 7.22-7.25 (m, 2H), 7.30-7.35 (m, 3H), 7.45-7.49 (m, 4H), 7.75 (d, J=8.4 Hz, 2H).

### Example 268

### Synthesis of 3-(7-(4-chlorophenyl)-5-(4-cyanophenyl)-8-hydroxy-6-oxo-5,7-diazaspiro[3.4]octan-8-yl)benzonitrile

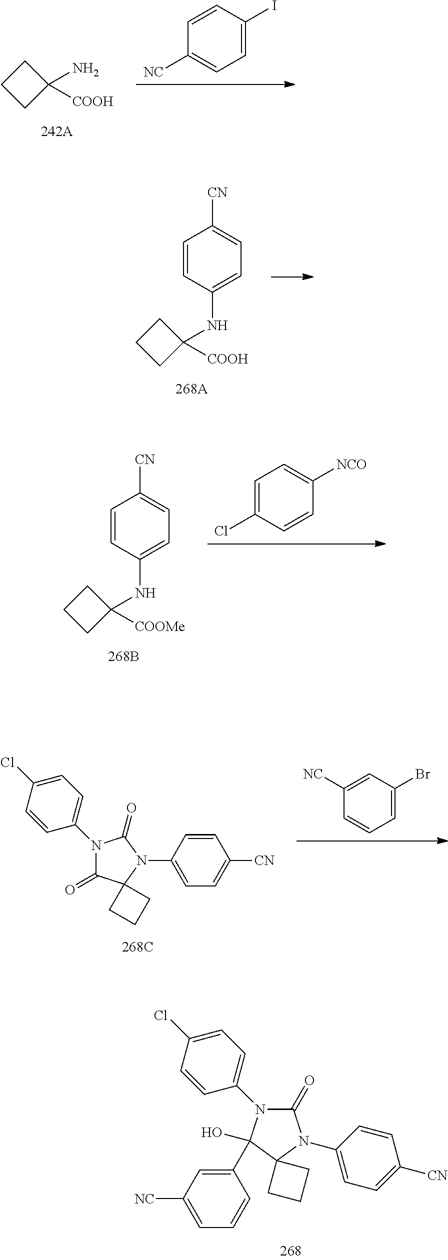

Compound 268A was synthesized by employing the procedure described for Compound 224B using Compound 242A in the presence of 2-acetylcyclohexan-1-one and 4-iodobenzonitrile in lieu of Compounds 224A without 2-acetylcyclohexan-1-one and 1-chloro-4-iodobenzene. LC-MS (ESI) m/z: 217 [M+H]⁺.

To a mixture of Compound 268A (6.2 g, 13.6 mmol), methanol (20 mL), and acetonitrile (150 mL) was added a solution of (diazomethyl)trimethylsilane in hexane (2M, 21.5 mL, 43 mmol). The mixture was stirred at room temperature for 2 hours and concentrated under vacuum. The residue was purified with flash column chromatography on silica gel (ethyl acetate in petroleum ether, from 17% v/v) to afford Compound 268B. LC-MS (ESI) m/z: 231 [M+H]⁺. ¹H-NMR (400 MHz, CDCl₃): δ (ppm) 2.08-2.14 (m, 2H), 2.19-2.25 (m, 2H), 2.73-2.79 (m, 2H), 3.71 (s, 3H), 4.79 (s, 1H), 6.41 (d, J=7.2 Hz, 2H), 7.40 (d, J=6.8 Hz, 2H).

Compounds 268C and 268 were synthesized by employing the procedures described for Compounds 224D and 125 using Compounds 268B, 268C, and 3-bromobenzonitrile in lieu of Compounds 224C, 2C, and 3-bromo-N,N-dimethylaniline. Compound 268C. LC-MS (ESI) m/z: 352 [M+H]⁺; ¹H-NMR (400 MHz, CDCl₃): δ (ppm) 1.76-1.84 (m, 1H), 2.24-2.31 (s, 1H), 2.49-2.56 (m, 2H), 2.66-2.71 (m, 2H), 7.44-7.47 (m, 4H), 7.56-7.58 (m, 2H), 7.82-7.83 (m, 2H). Compound 268. LC-MS (ESI) m/z: 455 [M+H]⁺. ¹H-NMR (400 MHz, CDCl₃): δ (ppm) 0.82-0.88 (m, 1H), 1.17-1.22 (m, 1H), 1.73-1.84 (m, 1H), 2.01-2.09 (m, 1H), 2.29-2.37 (m, 1H), 2.73-2.80 (m, 1H), 4.27 (s, 1H), 7.16-7.19 (m, 2H), 7.31-7.34 (m, 2H), 7.44-7.48 (m, 3H), 7.63-7.65 (m, 2H), 7.70-7.75 (m, 2H), 7.91-7.96 (m, 1H).

### Example 269

### Synthesis of 4-(7-(4-chlorophenyl)-8-hydroxy-6-oxo-8-(3-(trifluoromethoxy)phenyl)-5,7-diazaspiro[3.4]octan-5-yl)benzonitrile

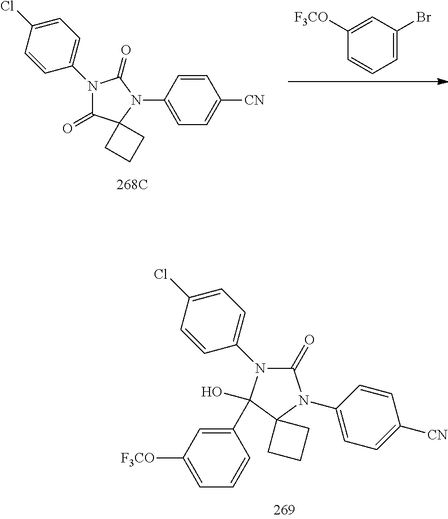 [SMILES: N#Cc1ccc(N2C(=O)N(c3ccc(Cl)cc3)C(=O)C23CCC3)cc1 ; FC(F)(F)Oc1cccc(Br)c1 ; N#Cc1ccc(N2C(=O)N(c3ccc(Cl)cc3)C(O)(c3cccc(OC(F)(F)F)c3)C23CCC3)cc1]

Compound 269 was synthesized by employing the procedure described for Compound 125 using Compound 268C and 1-bromo-3-(trifluoromethoxy)benzene in lieu of Compound 2C and 3-bromo-N,N-dimethylaniline. Compound 269. LC-MS (ESI) m/z: 514 [M+H]⁺. ¹H-NMR (400 MHz, CDCl₃): δ (ppm) 0.83-0.95 (m, 1H), 1.12-1.23 (m, 1H), 1.78-1.86 (m, 1H), 1.98-2.05 (m, 1H), 2.25-2.33 (m, 1H), 2.72-2.80 (m, 1H), 4.25 (s, 1H), 7.16-7.21 (m, 3H), 7.32-7.38 (m, 4H), 7.42-7.44 (m, 3H), 7.68-7.70 (m, 2H).

### Example 270

### Synthesis of 3-(1-(4-cyanophenyl)-4-hydroxy-5,5-dimethyl-2-oxo-3-(p-tolyl)imidazolidin-4-yl)benzonitrile

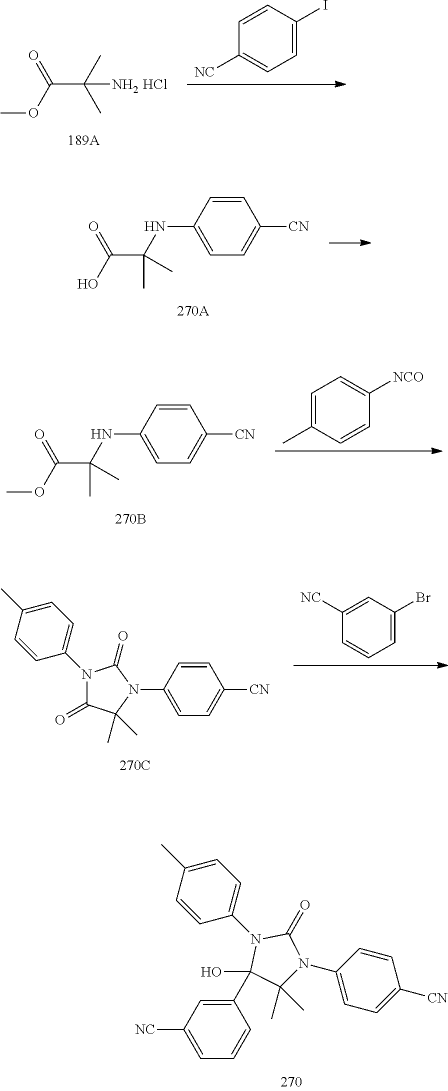

Compounds 270A, 270B, 270C, and 270 were synthesized by employing the procedures described for Compounds 224B, 268B, 224D, and 125 using Compounds 189A in the presence of 2-acetylcyclohexan-1-one, 4-iodobenzonitrile, 270A, 270B, 1-isocyanato-4-methylbenzene, 270C, and 3-bromobenzonitrile in lieu of Compounds 224A without 2-acetylcyclohexan-1-one, 1-chloro-4-iodobenzene, 268A, 224C, 1-chloro-4-isocyanatobenzene, 2C, and 3-bromo-N,N-dimethylaniline. Compound 270A. LC-MS (ESI) m/z: 205 [M+H]⁺. Compound 270B. LC-MS (ESI) m/z: 219 [M+H]⁺. ¹H-NMR (CDCl₃, 400 MHz): δ (ppm) 1.60 (s, 6H), 3.73 (s, 3H), 4.62 (s, 1H), 6.49 (d, J=9.2 Hz, 2H), 7.40 (d, J=8.8 Hz, 2H). Compound 270C. LC-MS (ESI) m/z: 320 [M+H]⁺; ¹H-NMR (CDCl₃, 400 MHz): δ (ppm) 1.62 (s, 6H), 2.40 (s, 3H), 7.28-7.34 (m, 4H), 7.54-7.55 (m, 2H), 7.75-7.77 (m, 2H). Compound 270. LC-MS (ESI) m/z: 423 [M+H]⁺. ¹H-NMR (CDCl₃, 400 MHz): δ (ppm) 0.83 (s, 3H), 1.36 (s, 3H), 2.29 (s, 3H), 4.42 (s, 1H), 7.04 (d, J=6.8 Hz, 2H), 7.26 (d, J=6.4 Hz, 2H), 7.35-7.40 (m, 3H), 7.57-7.62 (m, 4H), 7.80 (s, 1H).

### Example 271

### Synthesis of 5,7-bis(4-chlorophenyl)-8-(3-(1,1-difluoroethyl)phenyl)-8-hydroxy-2-oxa-5,7-diazaspiro[3.4]octan-6-one

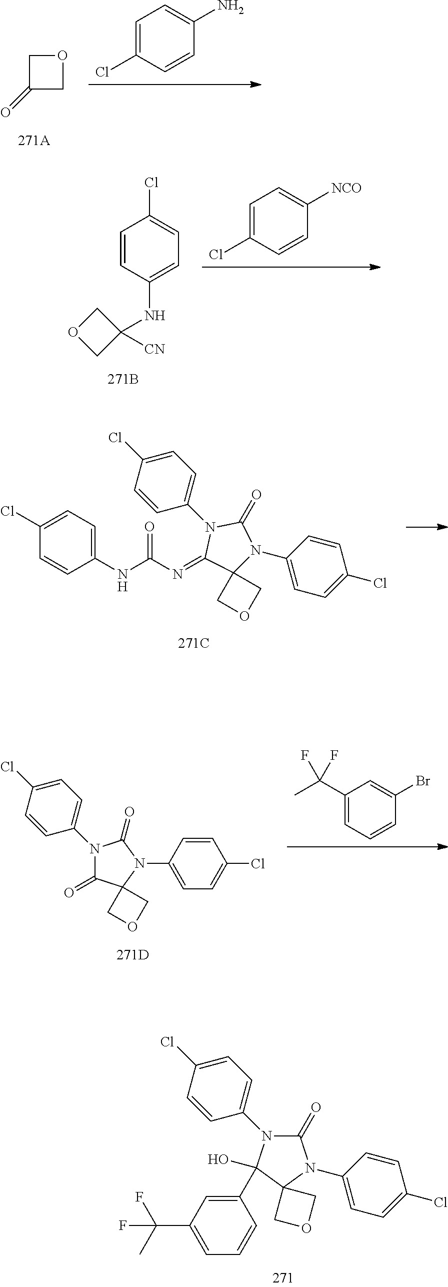

A mixture of 4-chloroaniline (2.54 g, 20 mmol), oxetan-3-one (2.88 g, 40 mmol), and anhydrous sodium sulfate (20 g) in dichloromethane (100 mL) was stirred at room temperature for 2 hours. To the mixture at room temperature was added TMSCN (7.5 mL, 40 mmol), followed by addition of BF₃-Et₂O (5 mL) at 0° C. The resulting mixture was stirred at room temperature for 16 hours and filtered. The filtrate was concentrated and purified with flash column chromatography on silica gel (ethyl acetate in petroleum ether, 18% v/v) to furnish Compound 271B. LC-MS (ESI) m/z: 209 [M+H]⁺; ¹H-NMR (CDCl₃, 400 MHz): δ (ppm) 4.36 (s, 1H), 4.72 (d, J=6.4 Hz, 2H), 5.12 (d, J=6.4 Hz, 2H), 6.48 (d, J=9.2 Hz, 2H), 7.25 (d, J=9.2 Hz, 2H).

To a solution of Compound 271B (200 mg, 0.96 mmol) in anhydrous pyridine (5 mL) at 80° C. was added 1-chloro-4-isocyanatobenzene (294 mg, 1.92 mmol). The mixture was stirred at 80° C. for 3 hours, and then another portion of 1-chloro-4-isocyanatobenzene (147 mg, 0.96 mmol) was added. The resulting mixture was stirred at 80° C. for another hour and evaporated to remove most of pyridine under reduced pressure. The residue was triturated with ethyl acetate (16 mL), filtered, and dried to afford a pure Compound 271C. LC-MS (ESI) m/z: 515 [M+H]⁺.

To a suspension of Compound 271C (285 mg, 0.60 mmol) in THF (10 mL) and methanol (10 mL) at room temperature was slowly added a diluted H₂SO₄ solution (2 M, 2.5 mL). The mixture was stirred at room temperature for 3 hours and evaporated under the reduced pressure. The residue was diluted with water (10 mL) and extracted with ethyl acetate (50 mL×3). The combined organic layers was washed with brine (10 mL), dried over anhydrous sodium sulfate, filtered, and concentrated to leave a crude product, which was triturated with ethyl acetate (10 mL), filtered, and dried to afford a pure Compound 271D. LC-MS (ESI) m/z: 363 [M+H]⁺; ¹H-NMR (DMSO-d₆, 400 MHz): δ (ppm) 4.71 (d, J=7.6 Hz, 2H), 4.87 (d, J=7.6 Hz, 2H), 7.49 (d, J=8.8 Hz, 2H), 7.63 (d, J=8.8 Hz, 2H), 7.56-7.68 (m, 4H).

Compound 271 was synthesized by employing the procedure described for Compound 125 using Compound 271D and 1-bromo-3-(1,1-difluoroethyl)benzene in lieu of Compound 2C and 3-bromo-N,N-dimethylaniline. Compound 271. LC-MS (ESI) m/z: 505 [M+H]⁺; ¹H-NMR (CDCl₃, 400 MHz): δ (ppm) 1.82 (t, J=18 Hz, 3H), 3.93 (d, J=7.6 Hz, 1H), 4.37 (d, J=7.6 Hz, 1H), 4.55 (d, J=8.4 Hz, 1H), 5.12 (d, J=7.6 Hz, 1H), 5.23 (s, 1H), 7.13 (d, J=8.4 Hz, 2H), 7.17 (s, 4H), 7.35 (d, J=8.0 Hz, 2H), 7.41-7.52 (m, 4H).

### Example 272

### Synthesis of 5,7-bis(4-chlorophenyl)-8-(3-(difluoromethoxy)phenyl)-8-hydroxy-2-oxa-5,7-diazaspiro[3.4]octan-6-one

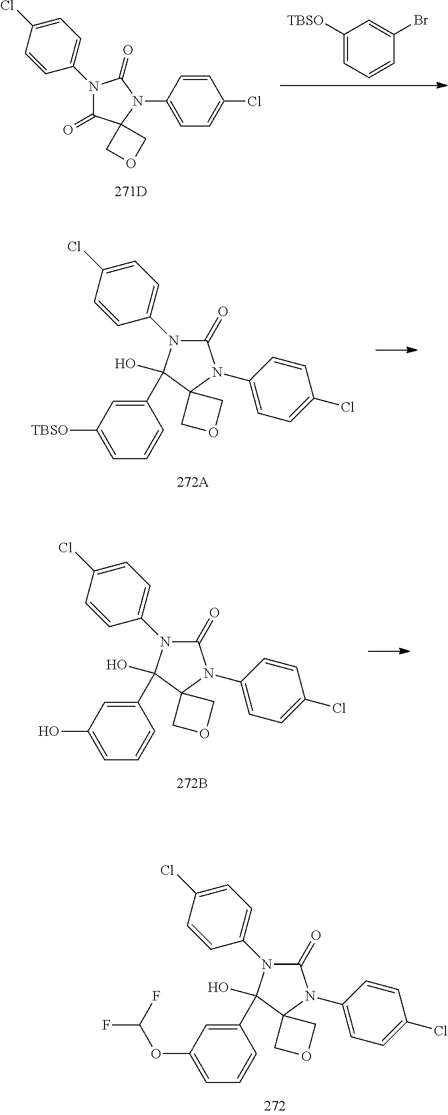 [SMILES: O=C1N(c2ccc(Cl)cc2)C(=O)C2(COC2)N1c1ccc(Cl)cc1 ; CC(C)(C)[Si](C)(C)Oc1cccc(Br)c1 ; CC(C)(C)[Si](C)(C)Oc1cccc(C2(O)N(c3ccc(Cl)cc3)C(=O)N(c3ccc(Cl)cc3)C23COC3)c1 ; O=C1N(c2ccc(Cl)cc2)C2(COC2)C(O)(c2cccc(O)c2)N1c1ccc(Cl)cc1 ; O=C1N(c2ccc(Cl)cc2)C2(COC2)C(O)(c2cccc(OC(F)F)c2)N1c1ccc(Cl)cc1]

Compounds 272A and 272B were synthesized by employing the procedures described for Compounds 125 and 227 using (3-bromophenoxy)(tert-butyl)dimethylsilane, Compounds 271D and 272A in lieu of 3-bromo-N,N-dimethylaniline, Compounds 2C, and 227A. Compound 272A. LC-MS (ESI) m/z: 571 [M+H]⁺; ¹H-NMR (CDCl₃, 400 MHz): δ (ppm) 0.07 (d, J=2.8 Hz, 6H), 0.93 (s, 9H), 4.11 (d, J=7.6 Hz, 1H), 4.14 (s, 1H), 4.44 (d, J=7.6 Hz, 1H), 4.66 (d, J=8.4 Hz, 1H), 5.22 (d, J=8.4 Hz, 1H), 6.84 (d, J=6.0 Hz, 2H), 7.12 (s, 1H), 7.17 (d, J=8.8 Hz, 2H), 7.25-7.35 (m, 5H), 7.42 (dd, J=8.8 Hz, 2H). Compound 272B. LC-MS (ESI) m/z: 457 [M+H]⁺.

A mixture of compound 272B (100 mg, 0.2187 mmol), sodium 2-chloro-2,2-difluoroacetate (84 mg, 0.5468 mmol), and K₂CO₃ (60 mg, 0.4374 mmol) in DMF (10 mL) was stirred at 100° C. for 2 hours. After cooled down to room temperature, the mixture was diluted with ethyl acetate (30 mL) and washed with brine (20 mL×3). The organic layers was dried over anhydrous sodium sulfate, filtered, and evaporated under the reduced pressure. The residue was purified with preparative HPLC to afford Compound 272. LC-MS (ESI) m/z: 507 [M+H]⁺; ¹H-NMR (CDCl₃, 400 MHz): δ (ppm) 3.99 (s, 1H), 4.10 (d, J=8.4 Hz, 1H), 4.50 (d, J=8.0 Hz, 1H), 4.73 (d, J=8.0 Hz, 1H), 5.22 (d, J=8.4 Hz, 1H), 6.50 (t, J=73.2 Hz, 1H), 7.14-7.48 (m, 12H).

### Example 273

### Synthesis of 4-(3-(4-chlorophenyl)-5-hydroxy-4,4-dimethyl-2-oxo-5-(3-(trifluoromethoxy)phenyl)imidazolidin-1-yl)benzonitrile

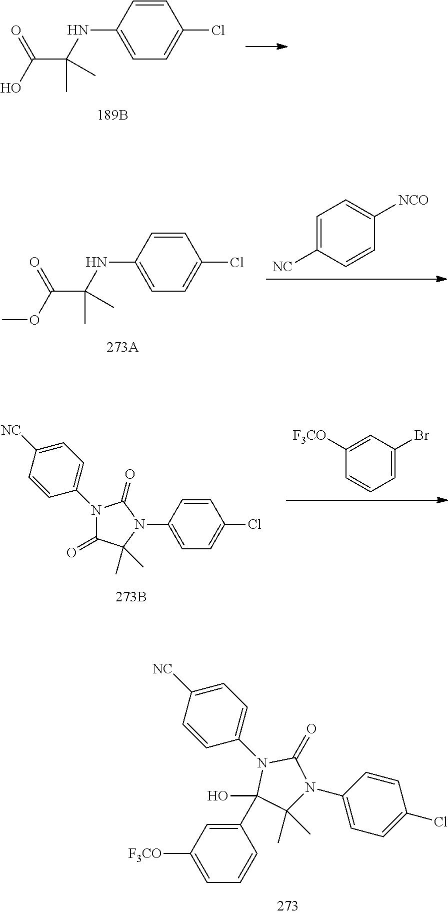

Compounds 273A, 273B, and 273 were synthesized by employing the procedures described for Compounds 224C, 224D, and 125 using Compounds 189B, 273A, 4-isocyanatobenzonitrile, 273B, and 1-bromo-3-(trifluoromethoxy)benzene in lieu of Compounds 224B, 224C, 1-chloro-4-isocyanatobenzene, 2C, and 3-bromo-N,N-dimethylaniline. Compound 273A. LC-MS (ESI) m/z: 228 [M+H]⁺. Compound 273B. LC-MS (ESI) m/z: 340 [M+H]⁺; ¹H-NMR (400 MHz, CDCl₃): δ (ppm) 1.56 (s, 3H), 1.58 (s, 3H), 7.25-7.26 (m, 2H), 7.46-7.48 (m, 2H), 7.73-7.78 (m, 4H). Compound 273. LC-MS (ESI) m/z: 502 [M+H]⁺; ¹H-NMR (400 MHz, CDCl₃): δ (ppm) 0.78 (s, 3H), 1.29 (s, 3H), 4.57 (s, 1H), 7.10-7.19 (m, 4H), 7.23-7.26 (m, 4H), 7.43-7.45 (m, 2H), 7.52-7.54 (m, 2H).

### Example 274

### Synthesis of 4-(5-(4-chlorophenyl)-8-hydroxy-6-oxo-8-(3-(trifluoromethoxy)phenyl)-2-oxa-5,7-diazaspiro[3.4]octan-7-yl)benzonitrile

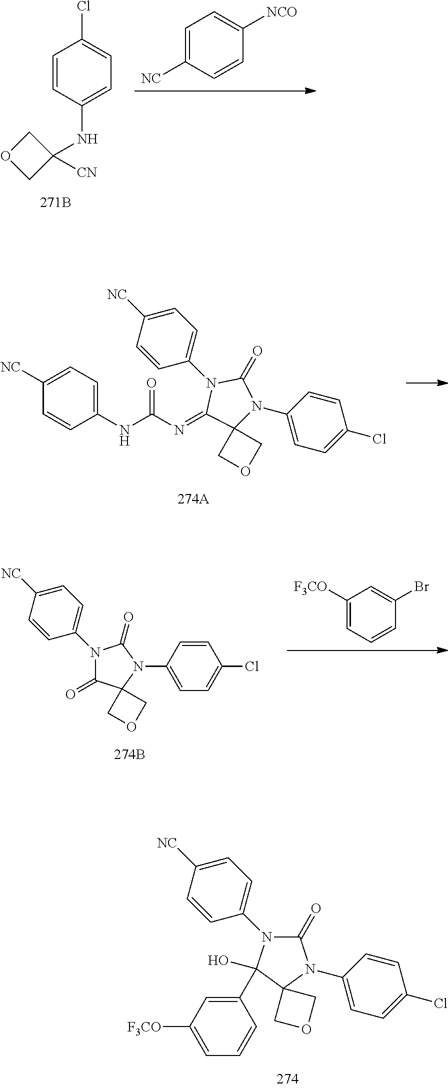

Compounds 274A, 274B, and 274 were synthesized by employing the procedures described for Compounds 271C, 271D, and 125 using 4-isocyanatobenzonitrile, Compounds 274A, 274B, and 1-bromo-3-(trifluoromethoxy)benzene in lieu of 1-chloro-4-isocyanatobenzene, Compounds 271C, 2C, and 3-bromo-N,N-dimethylaniline. Compound 274A. LC-MS (ESI) m/z: 497 [M+H]⁺; ¹H-NMR (DMSO-d₆, 500 MHz): δ (ppm) 4.79 (d, J=6.4 Hz, 2H), 5.07 (d, J=6.0 Hz, 2H), 7.46 (d, J=6.4 Hz, 2H), 7.59 (d, J=6.4 Hz, 2H), 7.65-7.71 (m, 6H), 7.83 (d, J=6.8 Hz, 2H), 10.16 (s, 1H). Compound 274B. LC-MS (ESI) m/z: 354 [M+H]⁺; ¹H-NMR (DMSO-d₆, 500 MHz): δ (ppm) 4.68 (d, J=6.0 Hz, 2H), 4.85 (d, J=6.4 Hz, 2H), 7.66 (s, 4H), 7.71 (t, J=5.6 Hz, 2H), 8.01 (q, J=4 Hz, 2H). Compound 274. LC-MS (ESI) m/z: 516 [M+H]⁺; ¹H-NMR (DMSO-d₆, 400 MHz): δ (ppm) 3.73 (d, J=8.4 Hz, 1H), 4.42 (d, J=8.0 Hz, 1H), 4.53 (d, J=8.4 Hz, 1H), 5.08 (d, J=8.4 Hz, 1H), 7.35-7.69 (m, 12H), 8.04 (s, 1H).

### Example 275

### Synthesis of 4,4′-(8-hydroxy-6-oxo-8-(3-(trifluoromethoxy)phenyl)-2-oxa-5,7-diazaspiro[3.4]octane-5,7-diyl)dibenzonitrile

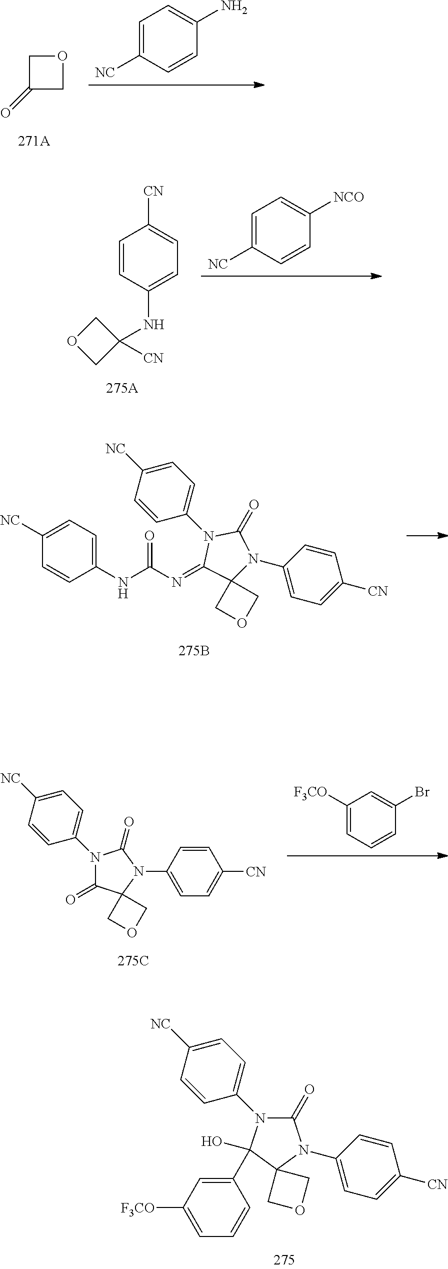

Compounds 275A, 275B, 275C, and 275 were synthesized by employing the procedures described for Compounds 271B, 271C, 271D, and 125 using 4-aminobenzonitrile, 4-isocyanatobenzonitrile, Compounds 275A, 275B, 275C, and 1-bromo-3-(trifluoromethoxy)benzene in lieu of 4-chloroaniline, 1-chloro-4-isocyanatobenzene, Compounds 271B, 271C, 2C, and 3-bromo-N,N-dimethylaniline. Compound 275A. LC-MS (ESI) m/z: 200 [M+H]⁺; ¹H-NMR (CDCl₃, 400 MHz): δ (ppm) 4.73 (d, J=6.8 Hz, 2H), 4.93 (s, 1H), 5.17 (d, J=6.8 Hz, 2H), 6.55-6.58 (m, 2H), 7.56-7.58 (m, 2H). Compound 275B. LC-MS (ESI) m/z: 488 [M+H]⁺. Compound 275C. LC-MS (ESI) m/z: 345 [M+H]⁺; ¹H-NMR (DMSO-d₆, 400 MHz): δ (ppm) 4.78 (d, J=8.4 Hz, 2H), 4.89 (d, J=8.4 Hz, 2H), 7.69-7.71 (m, 2H), 7.95-7.97 (m, 2H), 8.02-8.08 (m, 4H). Compound 275. LC-MS (ESI) m/z: 507 [M+H]⁺; ¹H-NMR (CDCl₃, 400 MHz): δ (ppm) 4.23 (d, J=8.4 Hz, 1H), 4.53 (s, 1H), 4.65 (d, J=8.4 Hz, 1H), 4.82 (d, J=8.8 Hz, 1H), 5.25 (d, J=8.8 Hz, 1H), 7.26-7.34 (m, 2H), 7.42-7.54 (m, 6H), 7.70 (d, J=8.4 Hz, 2H), 7.82 (d, J=8.8 Hz, 2H).

### Example 276

### Synthesis of 4-(7-(4-chlorophenyl)-8-hydroxy-6-oxo-8-(3-(trifluoromethoxy)phenyl)-2-oxa-5,7-diazaspiro[3.4]octan-5-yl)benzonitrile

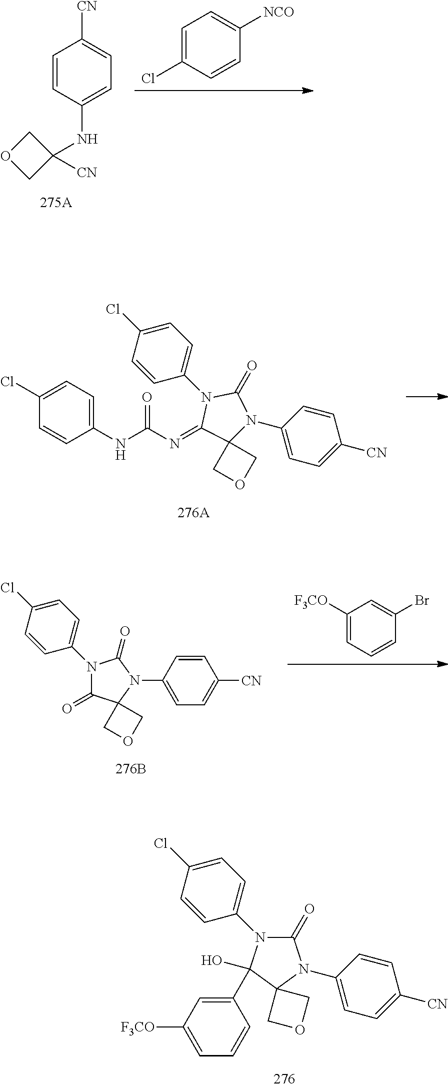

Compounds 276A, 276B, and 276 were synthesized by employing the procedures described for Compounds 271C, 271D, and 125 using Compounds 275A, 276A, 276B, and 1-bromo-3-(trifluoromethoxy)benzene in lieu of Compounds 271B, 271C, 2C, and 3-bromo-N,N-dimethylaniline. Compound 276A. LC-MS (ESI) m/z: 506 [M+H]⁺. Compound 276B. LC-MS (ESI) m/z: 354 [M+H]⁺. Compound 276. LC-MS (ESI) m/z: 516 [M+H]⁺; ¹H-NMR (CD₃OD, 500 MHz): 4.12 (d, J=8.5 Hz, 1H), 4.64 (d, J=8.5 Hz, 1H), 4.77 (d, J=8.5 Hz, 1H), 5.27 (d, J=9.0 Hz, 1H), 7.23 (d, J=15 Hz, 2H), 7.30 (d, J=8.0 Hz, 1H), 7.47-7.54 (m, 4H), 7.60-7.63 (m, 1H), 7.91-7.95 (m, 4H).

### Example 277

### Synthesis of 4-(8-hydroxy-6-oxo-7-(p-tolyl)-8-(3-(trifluoromethoxy)phenyl)-2-oxa-5,7-diazaspiro[3.4]octan-5-yl)benzonitrile

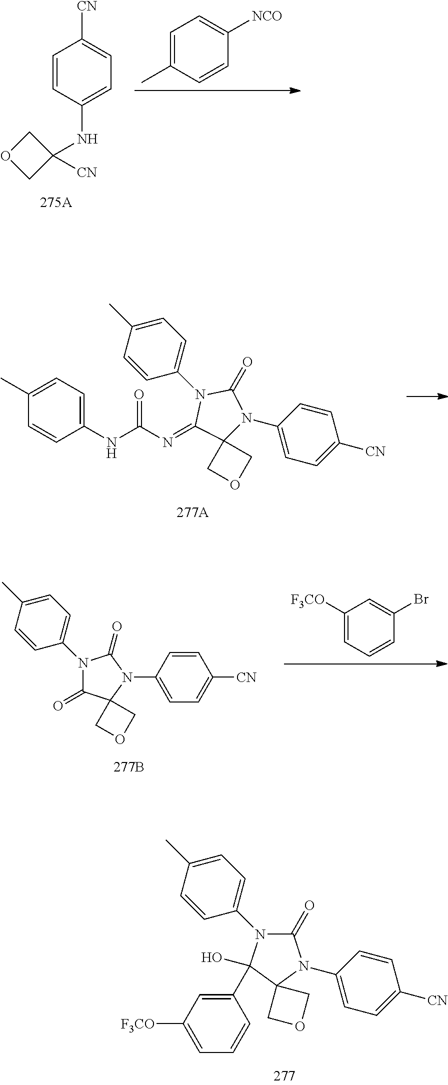

Compounds 277A, 277B, and 277 were synthesized by employing the procedures described for Compounds 271C, 271D, and 125 using 1-isocyanato-4-methylbenzene, Compounds 275A, 277A, 277B, and 1-bromo-3-(trifluoromethoxy)benzene in lieu of 1-chloro-4-isocyanatobenzene, Compounds 271B, 271C, 2C, and 3-bromo-N,N-dimethylaniline. Compound 277A. LC-MS (ESI) m/z: 466 [M+H]⁺. Compound 277B. LC-MS (ESI) m/z: 334 [M+H]⁺; ¹H-NMR (CDCl₃, 400 MHz): δ (ppm) 2.41 (s, 3H), 5.03 (d, J=7.6 Hz, 2H), 5.25 (d, J=8.0 Hz, 2H), 7.31 (s, 4H), 7.82 (d, J=8.8 Hz, 2H), 8.05 (d, J=8.8 Hz, 2H). Compound 277. LC-MS (ESI) m/z: 496 [M+H]⁺; ¹H-NMR (CDCl₃, 400 MHz): δ (ppm) 2.27 (s, 3H), 4.15 (s, 1H), 4.20 (d, J=8.0 Hz, 1H), 4.58 (d, J=8.4 Hz, 1H), 4.77 (d, J=8.8 Hz, 1H), 5.26 (d, J=8.8 Hz, 1H), 7.04 (d, J=8.0 Hz, 2H), 7.17 (d, J=8.4 Hz, 2H), 7.22-7.25 (m, 1H), 7.38 (s, 1H), 7.44 (d, J=4.8 Hz, 2H), 7.68-7.71 (m, 2H), 7.77-7.79 (m, 2H).

### Example 278

### Synthesis of 3-(5-(4-cyanophenyl)-8-hydroxy-6-oxo-7-(p-tolyl)-2-oxa-5,7-diazaspiro[3.4]octan-8-yl)benzonitrile

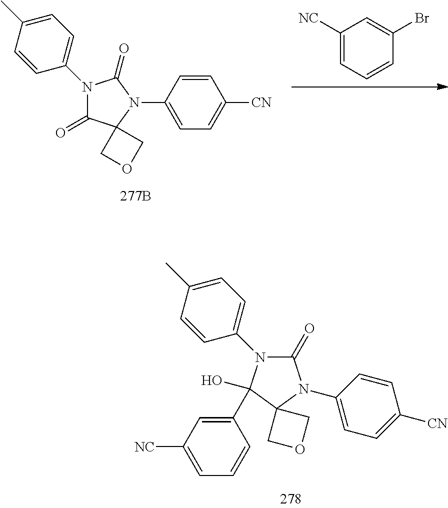 [SMILES: Cc1ccc(N2C(=O)N(c3ccc(C#N)cc3)C3(COC3)C2=O)cc1 ; N#Cc1cccc(Br)c1 ; Cc1ccc(N2C(=O)N(c3ccc(C#N)cc3)C3(COC3)C2(O)c2cccc(C#N)c2)cc1]

Compound 278 was synthesized by employing the procedure described for Compound 125 using Compound 277B and 3-bromobenzonitrile in lieu of Compound 2C and 3-bromo-N,N-dimethylaniline. Compound 278. LC-MS (ESI) m/z: 437 [M+H]⁺; ¹H-NMR (CDCl₃, 400 MHz): δ (ppm) 2.28 (s, 3H), 4.09 (d, J=8.0 Hz, 1H), 4.57-4.60 (m, 2H), 4.75 (d, J=8.8 Hz, 1H), 5.21 (d, J=8.8 Hz, 1H), 7.04 (d, J=8.4 Hz, 2H), 7.17 (d, J=8.4 Hz, 2H), 7.49 (t, J=7.6 Hz, 1H), 7.65-7.74 (m, 6H), 7.89 (s, 1H).

### Example 279

### Synthesis of 4-(8-hydroxy-6-oxo-7-(p-tolyl)-8-(3-(trifluoromethoxy)phenyl)-5,7-diazaspiro[3.4]octan-5-yl)benzonitrile

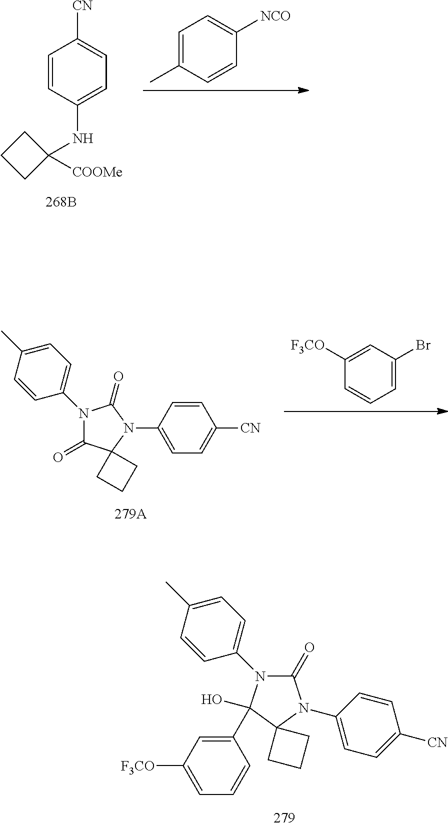

Compounds 279A and 279 were synthesized by employing the procedures described for Compounds 224D and 125 using 1-isocyanato-4-methylbenzene, Compounds 268B, 279A, and 1-bromo-3-(trifluoromethoxy)benzene in lieu of 1-chloro-4-isocyanatobenzene, Compounds 224C, 2C, and 3-bromo-N,N-dimethylaniline. Compound 279A. LC-MS (ESI) m/z: 332 [M+H]⁺. Compound 279. LC-MS (ESI) m/z: 494 [M+H]⁺. ¹H-NMR (CDCl₃, 400 MHz): δ (ppm) 0.80-0.90 (m, 1H), 1.17-1.24 (m, 1H), 1.83-1.89 (m, 1H), 1.98-2.08 (m, 1H), 2.25 (s, 3H), 2.30-2.38 (m, 1H), 2.76-2.84 (m, 1H), 3.74 (s, 1H), 6.97-7.02 (m, 2H), 7.13-7.36 (m, 3H), 7.36-7.52 (m, 5H), 7.72-7.75 (m, 2H).

### Example 280

### Synthesis of 3-(5-(4-cyanophenyl)-8-hydroxy-6-oxo-7-(p-tolyl)-5,7-diazaspiro[3.4]octan-8-yl)benzonitrile

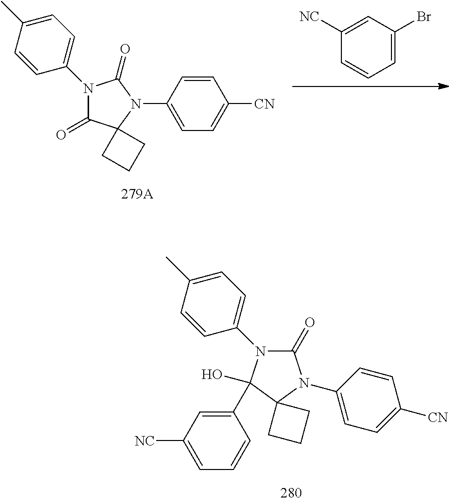 [SMILES: Cc1ccc(N2C(=O)N(c3ccc(C#N)cc3)C3(CCC3)C2=O)cc1 ; N#Cc1cccc(Br)c1 ; Cc1ccc(N2C(=O)N(c3ccc(C#N)cc3)C3(CCC3)C2(O)c2cccc(C#N)c2)cc1]

Compound 280 was synthesized by employing the procedure described for Compound 125 using Compound 279A and 3-bromobenzonitrile in lieu of Compound 2C and 3-bromo-N,N-dimethylaniline. Compound 280. LC-MS (ESI) m/z: 435 [M+H]⁺. ¹H-NMR (CDCl₃, 400 MHz): δ (ppm) 0.84-0.86 (m, 1H), 1.20-1.21 (m, 1H), 1.79-1.83 (m, 1H), 2.05-2.10 (m, 1H), 2.35 (s, 3H), 2.75-2.81 (m, 1H), 2.84-2.86 (m, 1H), 3.74 (s, 1H), 6.99-7.04 (m, 2H), 7.20-7.26 (m, 2H), 7.43-7.53 (m, 3H), 7.62-7.77 (m, 4H), 7.96 (s, 1H).

### Example 281

### Synthesis of 4-(8-hydroxy-6-oxo-5-(p-tolyl)-8-(3-(trifluoromethoxy)phenyl)-5,7-diazaspiro[3.4]octan-7-yl)benzonitrile

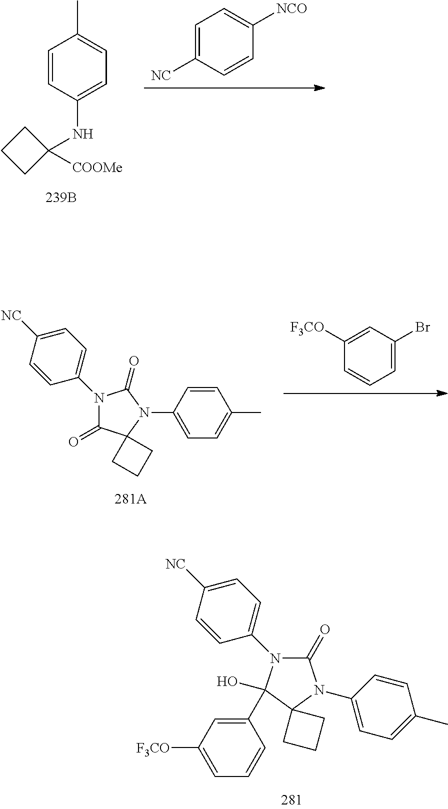

Compounds 281A and 281 were synthesized by employing the procedures described for Compounds 224D and 125 using 4-isocyanatobenzonitrile, Compounds 239B, 281A, and 1-bromo-3-(trifluoromethoxy)benzene in lieu of 1-chloro-4-isocyanatobenzene, Compounds 224C, 2C, and 3-bromo-N,N-dimethylaniline. Compound 281A. LC-MS (ESI) m/z: 332 [M+H]⁺. Compound 281. LC-MS (ESI) m/z: 494 [M+H]⁺; ¹H-NMR (CDCl₃, 400 MHz): δ (ppm) 0.82-0.94 (m, 1H), 1.08-1.18 (m, 1H), 1.72-1.80 (m, 1H), 2.05-2.15 (m, 1H), 2.27-2.37 (m, 1H), 2.42 (s, 3H), 2.67-2.77 (m, 1H), 3.59 (s, 1H), 7.19-7.25 (m, 4H), 7.28-7.33 (m, 2H), 7.35-7.49 (m, 4H), 7.57-7.62 (m, 2H).

### Example 282

### Synthesis of 3-(7-(4-cyanophenyl)-8-hydroxy-6-oxo-5-(p-tolyl)-5,7-diazaspiro[3.4]octan-8-yl)benzonitrile

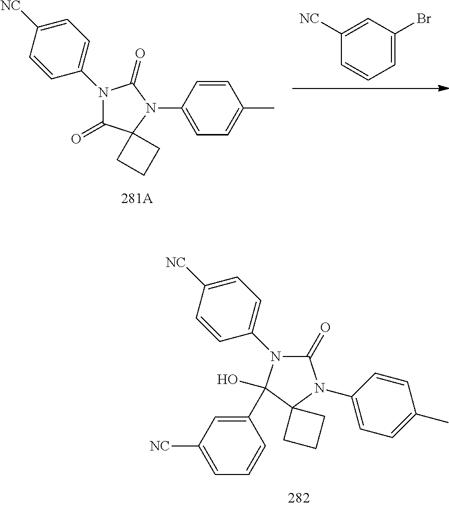 [SMILES: Cc1ccc(N2C(=O)N(c3ccc(C#N)cc3)C(=O)C23CCC3)cc1 ; N#Cc1cccc(Br)c1 ; Cc1ccc(N2C(=O)N(c3ccc(C#N)cc3)C(O)(c3cccc(C#N)c3)C23CCC3)cc1]

Compound 282 was synthesized by employing the procedure described for Compound 125 using Compound 281A and 3-bromobenzonitrile in lieu of Compound 2C and 3-bromo-N,N-dimethylaniline. Compound 282. LC-MS (ESI) m/z: 435 [M+H]⁺; ¹H-NMR (CDCl₃, 400 MHz): δ (ppm) 0.80-0.90 (m, 1H), 1.08-1.18 (m, 1H), 1.63-1.72 (m, 1H), 2.02-2.08 (m, 1H), 2.29-2.38 (m, 1H), 2.42 (s, 3H), 2.68-2.77 (m, 1H), 4.07 (s, 1H), 7.20-7.32 (m, 6H), 7.42-7.50 (m, 3H), 7.54-7.58 (m, 2H), 7.64-7.68 (m, 1H).

### Example 283

### Synthesis of 3-(7-(4-chlorophenyl)-8-hydroxy-6-oxo-5-(p-tolyl)-2-oxa-5,7-diazaspiro[3.4]octan-8-yl)benzonitrile

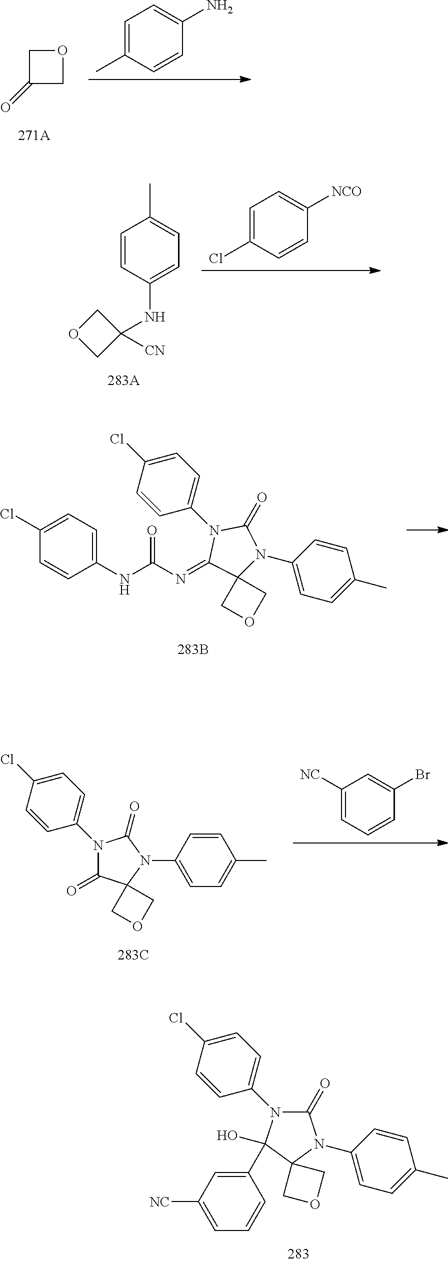

Compounds 283A, 283B, 283C, and 283 were synthesized by employing the procedures described for Compounds 271B, 271C, 271D, and 125 using p-toluidine, Compounds 282A, 283B, 283C, and 1-bromo-3-(trifluoromethoxy)benzene in lieu of 4-chloroaniline, Compounds 271B, 271C, 2C, and 3-bromo-N,N-dimethylaniline. Compound 283A. LC-MS (ESI) m/z: 189 [M+H]⁺; ¹H-NMR (CDCl₃, 400 MHz): δ (ppm) 2.28 (s, 3H), 4.21 (s, 1H), 4.72 (d, J=6.4 Hz, 2H), 5.12 (d, J=6.4 Hz, 2H), 6.46 (d, J=8.0 Hz, 2H), 7.08 (d, J=8.0 Hz, 2H). Compound 283B. LC-MS (ESI) m/z: 495 [M+H]⁺; ¹H-NMR (DMSO-d₆, 400 MHz): δ (ppm) 2.40 (s, 3H), 4.78 (d, J=7.6 Hz, 2H), 5.06 (d, J=7.6 Hz, 2H), 7.28-7.34 (m, 5H), 7.37-7.40 (m, 5H), 7.47-7.51 (m, 2H), 9.73 (s, 1H). Compound 283C. LC-MS (ESI) m/z: 343 [M+H]⁺; ¹H-NMR (DMSO-d₆, 400 MHz): δ (ppm) 2.39 (s, 3H), 4.67 (d, J=8.0 Hz, 2H), 4.84 (d, J=8.0 Hz, 2H), 7.38 (d, J=8.0 Hz, 2H), 7.46-7.51 (m, 4H), 7.59 (d, J=8.8 Hz, 2H). Compound 283. LC-MS (ESI) m/z: 446 [M+H]⁺; ¹H-NMR (CDCl₃, 400 MHz): δ (ppm) 2.42 (s, 3H), 3.88 (d, J=7.6 Hz, 1H), 4.51 (d, J=7.6 Hz, 1H), 4.69-4.71 (m, 2H), 5.12 (d, J=7.6 Hz, 1H), 7.15-7.17 (m, 4H), 7.25-7.30 (m, 4H), 7.45-7.66 (m, 3H), 7.89 (s, 1H).

### Example 284

### Synthesis of 8-hydroxy-5,7-di-p-tolyl-8-(3-(trifluoromethoxy)phenyl)-2-oxa-5,7-diazaspiro[3.4]octan-6-one

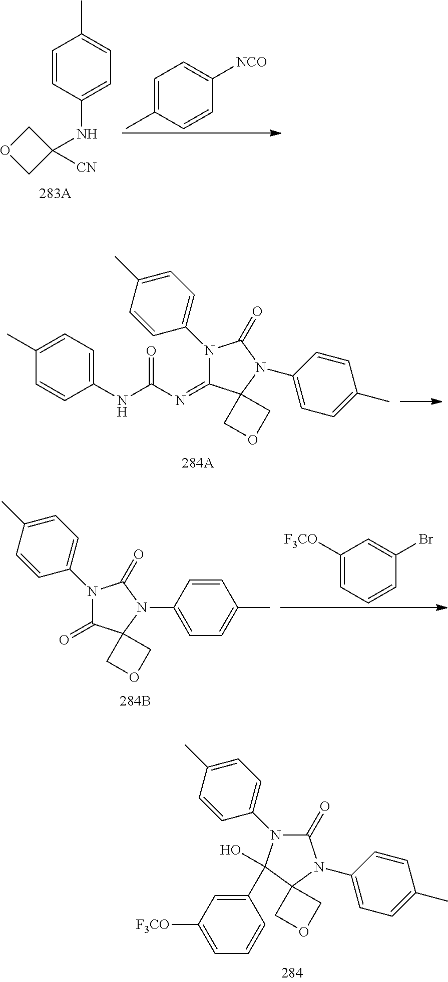

Compounds 284A, 284B, and 284 were synthesized by employing the procedures described for Compounds 271C, 271D, and 125 using 1-isocyanato-4-methylbenzene, Compounds 283A, 284A, 284B, and 1-bromo-3-(trifluoromethoxy)benzene in lieu of 1-chloro-4-isocyanatobenzene, Compounds 271B, 271C, 2C, and 3-bromo-N,N-dimethylaniline. Compound 284A. LC-MS (ESI) m/z: 455 [M+H]⁺. Compound 284B. LC-MS (ESI) m/z: 323 [M+H]⁺; ¹H-NMR (DMSO-d₆, 400 MHz): δ (ppm) 2.35 (s, 3H), 2.39 (s, 3H), 4.67 (d, J=7.6 Hz, 2H), 4.83 (d, J=7.6 Hz, 2H), 7.29-7.38 (m, 6H), 7.47-7.49 (m, 2H). Compound 284. LC-MS (ESI) m/z: 485 [M+H]⁺; ¹H-NMR (CDCl₃, 400 MHz): δ (ppm) 2.25 (s, 3H), 2.40 (s, 3H), 3.97 (s, 1H), 4.02 (d, J=8.0 Hz, 1H), 4.51 (d, J=7.6 Hz, 1H), 4.71 (d, J=8.0 Hz, 1H), 5.17 (d, J=8.0 Hz, 1H), 7.00 (d, J=8.4 Hz, 2H), 7.18-7.26 (m, 7H), 7.38-7.46 (m, 3H).

### Example 285

### Synthesis of 8-(3-(difluoromethyl)phenyl)-8-hydroxy-5,7-di-p-tolyl-2-oxa-5,7-diazaspiro[3.4]octan-6-one

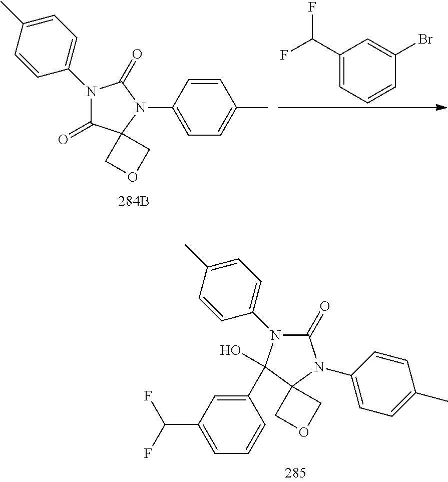

Compound 285 was synthesized by employing the procedure described for Compound 125 using Compound 284B and 1-bromo-3-(difluoromethyl)benzene in lieu of Compound 2C and 3-bromo-N,N-dimethylaniline. Compound 285. LC-MS (ESI) m/z: 451 [M+H]⁺; ¹H-NMR (CDCl₃, 400 MHz): δ (ppm) 2.24 (s, 3H), 2.39 (s, 3H), 3.97 (d, J=7.6 Hz, 1H), 4.24 (s, 1H), 4.49 (d, J=7.6 Hz, 1H), 4.69 (d, J=8.0 Hz, 1H), 5.18 (d, J=8.0 Hz, 1H), 6.63 (t, J=56.4 Hz, 1H), 6.98 (d, J=8.0 Hz, 2H), 7.18-7.26 (m, 6H), 7.42-7.58 (m, 3H), 7.74 (s, 1H).

### Example 286

### Synthesis of 8-(3-(1,1-difluoroethyl)phenyl)-8-hydroxy-5,7-di-p-tolyl-2-oxa-5,7-diazaspiro[3.4]octan-6-one

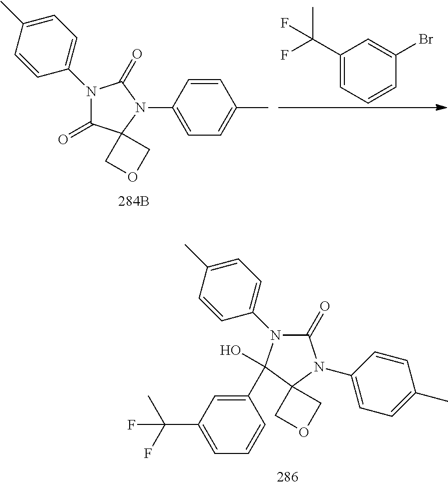

Compound 286 was synthesized by employing the procedure described for Compound 125 using Compound 284B and 1-bromo-3-(1,1-difluoroethyl)benzene in lieu of Compound 2C and 3-bromo-N,N-dimethylaniline. Compound 286. LC-MS (ESI) m/z: 465 [M+H]⁺; ¹H-NMR (CDCl₃, 400 MHz): δ (ppm) 1.82 (t, J=18.0 Hz, 3H), 2.24 (s, 3H), 2.39 (s, 3H), 3.97 (s, 1H), 4.00 (d, J=7.6 Hz, 1H), 4.51 (d, J=7.6 Hz, 1H), 4.73 (d, J=8.0 Hz, 1H), 5.20 (d, J=8.0 Hz, 1H), 7.00 (d, J=8.4 Hz, 2H), 7.20 (d, J=8.4 Hz, 2H), 7.26 (s, 4H), 7.41-7.49 (m, 2H), 7.59 (d, J=7.6 Hz, 1H), 7.66 (s, 1H).

### Example 287

### Synthesis of 3-(8-hydroxy-6-oxo-5,7-di-p-tolyl-2-oxa-5,7-diazaspiro[3.4]octan-8-yl)benzonitrile

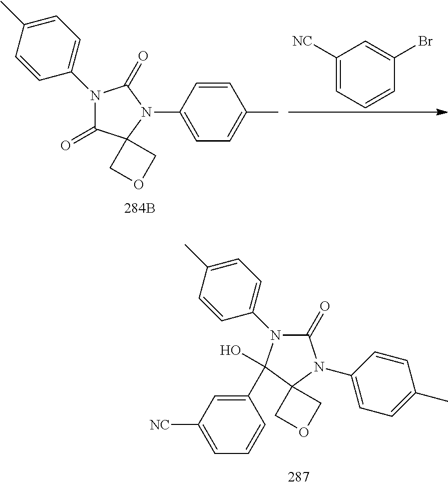

Compound 287 was synthesized by employing the procedure described for Compound 125 using Compound 284B and 3-bromobenzonitrile in lieu of Compound 2C and 3-bromo-N,N-dimethylaniline. Compound 287. LC-MS (ESI) m/z: 426 [M+H]⁺; ¹H-NMR (CDCl₃, 400 MHz): δ (ppm) 2.25 (s, 3H), 2.40 (s, 3H), 3.84 (d, J=8.0 Hz, 1H), 4.44 (d, J=7.6 Hz, 1H), 4.59 (d, J=8.0 Hz, 1H), 5.60 (d, J=8.4 Hz, 1H), 5.34 (s, 1H), 6.96 (d, J=8.0 Hz, 2H), 7.06-7.17 (m, 6H), 7.41 (d, J=8.0 Hz, 1H), 7.57 (d, J=7.6 Hz, 2H), 7.84 (s, 1H).

### Example 288

### Synthesis of 4-(5-(4-chlorophenyl)-8-hydroxy-6-oxo-8-(3-(trifluoromethoxy)phenyl)-5,7-diazaspiro[3.4]octan-7-yl)benzonitrile

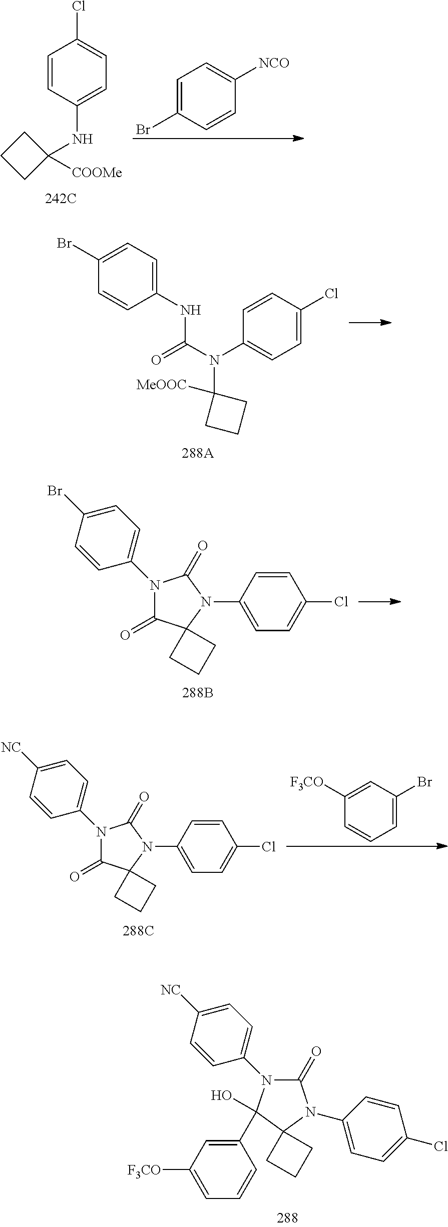

Compounds 288A, 288B, 288C, and 288 were synthesized by employing the procedures described for Compounds 224D, 224E, 180C, and 125 using 1-bromo-4-isocyanatobenzene, Compounds 242C, 288A, 288B, 288C, and 1-bromo-3-(trifluoromethoxy)benzene in lieu of 1-chloro-4-isocyanatobenzene, Compounds 224C, 224D, 180B, 2C, and 3-bromo-N,N-dimethylaniline. Compound 288A. LC-MS (ESI) m/z: 437 [M+H]⁺. Compound 288B. LC-MS (ESI) m/z: 405 [M+H]⁺. Compound 288C. LC-MS (ESI) m/z: 352 [M+H]⁺. Compound 288. LC-MS (ESI) m/z: 514 [M+H]⁺; ¹H-NMR (400 MHz, CDCl₃): ((ppm) 0.82-0.91 (m, 1H), 1.10-1.16 (m, 1H), 1.73-1.78 (m, 1H), 1.99-2.05 (m, 1H), 2.21-2.28 (m, 1H), 2.70-2.74 (m, 1H), 4.00 (s, 1H), 7.20-7.24 (m, 4H), 7.38-7.45 (m, 6H), 7.53-7.56 (m, 2H).

### Example 289

### Synthesis of 3-(5-(4-chlorophenyl)-7-(4-cyanophenyl)-8-hydroxy-6-oxo-5,7-diazaspiro[3.4]octan-8-yl)benzonitrile

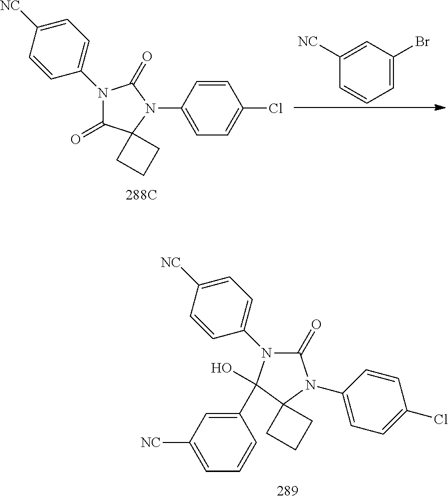 [SMILES: N#Cc1ccc(N2C(=O)N(c3ccc(Cl)cc3)C3(CCC3)C2=O)cc1 ; N#Cc1cccc(Br)c1 ; N#Cc1ccc(N2C(=O)N(c3ccc(Cl)cc3)C3(CCC3)C2(O)c2cccc(C#N)c2)cc1]

Compound 289 was synthesized by employing the procedure described for Compound 125 using Compound 288C and 3-bromobenzonitrile in lieu of Compound 2C and 3-bromo-N,N-dimethylaniline. Compound 289. LC-MS (ESI) m/z: 455 [M+H]⁺; ¹H-NMR (400 MHz, CDCl₃): δ (ppm) 0.79-0.88 (m, 1H), 1.06-1.16 (m, 1H), 1.60-1.65 (m, 1H), 1.98-2.05 (m, 1H), 2.19-2.26 (m, 1H), 2.68-2.73 (m, 1H), 4.38, 4.54 (s, 1H), 7.14-7.16 (m, 2H), 7.34-7.65 (m, 9H), 7.90-7.94 (m, 1H).

### Example 290

### Synthesis of 8-(3-(difluoromethoxy)phenyl)-8-hydroxy-5,7-di-p-tolyl-2-oxa-5,7-diazaspiro[3.4]octan-6-one

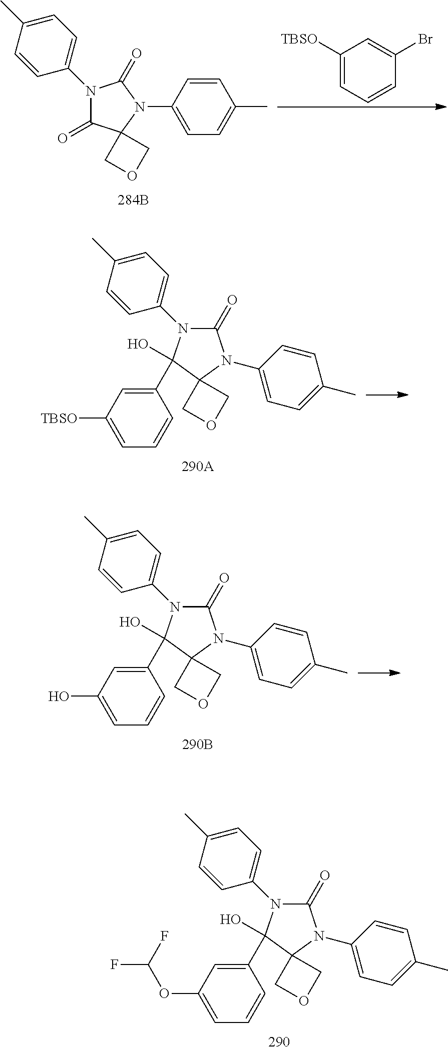

Compounds 290A and 290B were synthesized by employing the procedures described for Compounds 125 and 227 using (3-bromophenoxy)(tert-butyl)dimethylsilane, Compounds 284B, and 290A in lieu of 3-bromo-N,N-dimethylaniline, Compounds 2C, and 227A. Compound 290A. LC-MS (ESI) m/z: 531 [M+H]⁺; ¹H-NMR (CDCl₃, 400 MHz): δ (ppm) 0.00 (d, J=2.0 Hz, 6H), 0.87 (s, 9H), 2.17 (s, 3H), 2.33 (s, 3H), 3.62 (s, 1H), 4.04 (d, J=8.0 Hz, 1H), 4.43 (d, J=7.6 Hz, 1H), 4.65 (d, J=8.0 Hz, 1H), 5.16 (d, J=8.0 Hz, 1H), 6.73-6.76 (m, 1H), 6.86-6.98 (m, 4H), 7.14-7.24 (m, 7H). Compound 290B. LC-MS (ESI) m/z: 417 [M+H]⁺; ¹H-NMR (DMSO-d₆, 500 MHz): & (ppm) 2.19 (s, 3H), 2.37 (s, 3H), 3.81 (d, J=8.0 Hz, 1H), 4.32 (d, J=8.0 Hz, 1H), 4.50 (d, J=8.0 Hz, 1H), 5.06 (d, J=8.0 Hz, 1H), 6.68 (dd, J=8.0, 2.0 Hz, 1H), 6.87 (s, 1H), 6.96-7.01 (m, 3H), 7.14 (t, J=8.0 Hz, 1H), 7.32-7.36 (m, 5H), 7.42-7.44 (m, 2H), 9.47 (s, 1H).

The mixture of Compound 290B (100 mg, 0.24 mmol), sodium chlorodifluoroacetate (91 mg, 0.6 mmol) and cesium carbonate (157 mg, 0.48 mmol) in DMF (5 mL) was stirred at 100° C. for 2 hours. After cooled down to room temperature, the mixture was diluted with water (30 mL) and extracted with ethyl acetate (30 mL×2). The combined organic layers was washed with brine (30 mL), dried over anhydrous sodium sulfate, filtered, and concentrated. The residue was purified with preparative HPLC to yield Compound 290. LC-MS (ESI) m/z: 467 [M+H]⁺; ¹H-NMR (CDCl₃, 400 MHz): δ (ppm) 2.25 (s, 3H), 2.39 (s, 3H), 4.00-4.05 (m, 1H), 4.05, 4.35 (s, 1H), 4.51 (t, J=8.0 Hz, 1H), 4.67-4.73 (m, 1H), 5.18 (t, J=9.2 Hz, 1H), 6.43 (t, J=73.6 Hz, 1H), 6.99-7.02 (m, 2H), 7.07-7.10 (m, 1H), 7.19-7.25 (m, 6H), 7.31-7.38 (m, 3H).

### Example 291

### Synthesis of 3-(1,3-bis(4-chlorophenyl)-4-hydroxy-5-methyl-2-oxo-5-propylimidazolidin-4-yl)benzonitrile

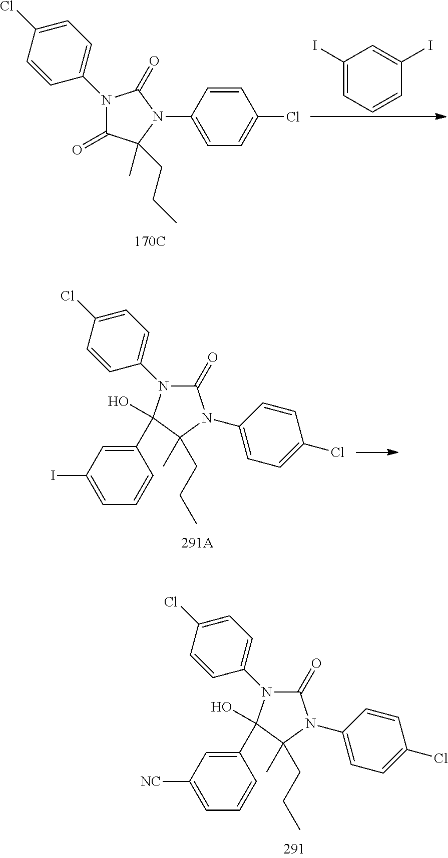 [SMILES: CCCC1(C)C(=O)N(c2ccc(Cl)cc2)C(=O)N1c1ccc(Cl)cc1 ; Ic1cccc(I)c1 ; CCCC1(C)N(c2ccc(Cl)cc2)C(=O)N(c2ccc(Cl)cc2)C1(O)c1cccc(I)c1 ; CCCC1(C)N(c2ccc(Cl)cc2)C(=O)N(c2ccc(Cl)cc2)C1(O)c1cccc(C#N)c1]

Compounds 291A and 291 were synthesized by employing the procedures described for Compounds 125 and 180C using 1,3-diiodobenzene, Compounds 170C, and 291A in lieu of 3-bromo-N,N-dimethylaniline, Compounds 2C, and 180B. Compound 291A. LC-MS (ESI) m/z: 581 [M+H]. Compound 291. LC-MS (ESI) m/z: 480 [M+H]; ¹H-NMR (400 MHz, CDCl₃): δ (ppm) 0.48, 0.87 (t, J=6.8 Hz, 3H), 0.85-0.88 (m, 1H), 0.93, 1.40 (s, 3H), 1.29-1.33 (m, 2H), 1.60-1.95 (m, 1H), 3.69, 3.75 (s, 1H), 7.15-7.22 (m, 4H), 7.27-7.45 (m, 5H), 7.60-7.65 (m, 2H), 7.87 (s, 1H).

### Example 292

### Synthesis of 4-(7-(4-bromophenyl)-8-hydroxy-6-oxo-8-(3-(trifluoromethoxy)phenyl)-5,7-diazaspiro[3.4]octan-5-yl)benzonitrile

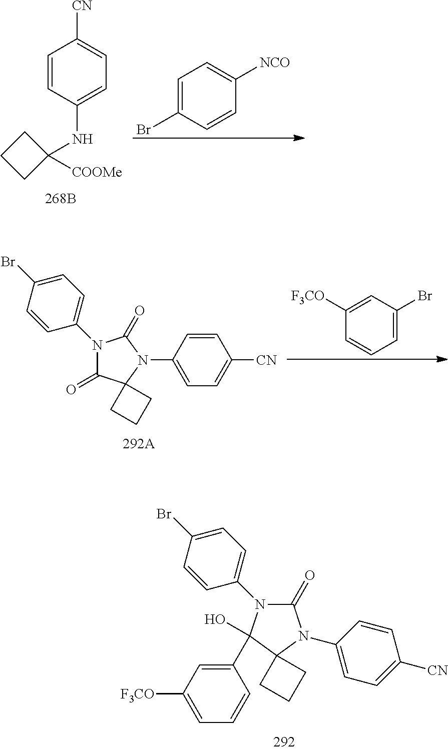

Compounds 292A and 292 were synthesized by employing the procedures described for Compounds 224D and 125 using 1-bromo-4-isocyanatobenzene, Compounds 268B, 292A, and 3-bromobenzonitrile in lieu of 1-chloro-4-isocyanatobenzene, Compounds 224C, 2C, and 3-bromo-N,N-dimethylaniline. Compound 292A. LC-MS (ESI) m/z: 396 [M+H]⁺; ¹H-NMR (400 MHz, CDCl₃): δ (ppm) 1.76-1.84 (m, 1H), 2.23-2.32 (m, 1H), 2.49-2.56 (m, 2H), 2.66-2.71 (m, 2H), 7.39-7.40 (m, 2H), 7.56-7.58 (m, 2H), 7.61-7.62 (m, 2H), 7.82-7.83 (m, 2H). Compound 292. LC-MS (ESI) m/z: 558 [M+H]⁺; ¹H-NMR (400 MHz, CDCl₃): δ (ppm) 0.85-0.94 (m, 1H), 1.16-1.24 (m, 1H), 1.81-1.88 (m, 1H), 2.00-2.08 (m, 1H), 2.29-2.37 (m, 1H), 2.74-2.82 (m, 1H), 3.86 (s, 1H), 7.20-7.22 (m, 1H), 7.28-7.34 (m, 4H), 7.37-7.44 (m, 3H), 7.46-7.49 (m, 2H), 7.73-7.75 (m, 2H).

### Example 293

### Synthesis of 4,4′-(8-hydroxy-6-oxo-8-(3-(trifluoromethoxy)phenyl)-5,7-diazaspiro[3.4]octane-5,7-diyl)dibenzonitrile

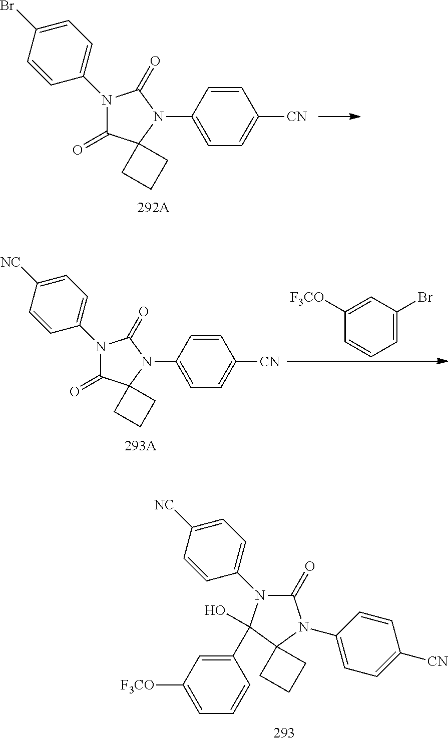 [SMILES: N#Cc1ccc(N2C(=O)N(c3ccc(Br)cc3)C(=O)C23CCC3)cc1 ; N#Cc1ccc(N2C(=O)N(c3ccc(C#N)cc3)C3(CCC3)C2=O)cc1 ; FC(F)(F)Oc1cccc(Br)c1 ; N#Cc1ccc(N2C(=O)N(c3ccc(C#N)cc3)C(O)(c3cccc(OC(F)(F)F)c3)C23CCC3)cc1]

Compounds 293A and 293 were synthesized by employing the procedures described for Compounds 180C and 125 using Compounds 292A, 293A, and 1-bromo-3-(trifluoromethoxy)benzene in lieu of Compounds 180B, 2C, and 3-bromo-N,N-dimethylaniline. Compound 293A. LC-MS (ESI) m/z: 343 [M+H]⁺; ¹H-NMR (400 MHz, CDCl₃): δ (ppm) 1.76-1.84 (m, 1H), 2.25-2.34 (m, 1H), 2.49-2.57 (m, 2H), 2.66-2.73 (m, 2H), 7.55-7.57 (m, 2H), 7.72-7.78 (m, 4H), 7.84-7.86 (m, 2H). Compound 293. LC-MS (ESI) m/z: 505 [M+H]⁺; ¹H-NMR (400 MHz, CDCl₃): δ (ppm) 0.88-1.00 (m, 1H), 1.18-1.28 (m, 1H), 1.81-1.89 (m, 1H), 2.00-2.11 (m, 1H), 2.30-2.37 (m, 1H), 2.76-2.83 (m, 1H), 4.19-4.23 (m, 1H), 7.23-7.24 (m, 1H), 7.39-7.44 (m, 5H), 7.49-7.55 (m, 4H), 7.73-7.75 (m, 2H).

### Example 294

### Synthesis of 4,4′-(8-(3-cyanophenyl)-8-hydroxy-6-oxo-5,7-diazaspiro[3.4]octane-5,7-diyl)dibenzonitrile

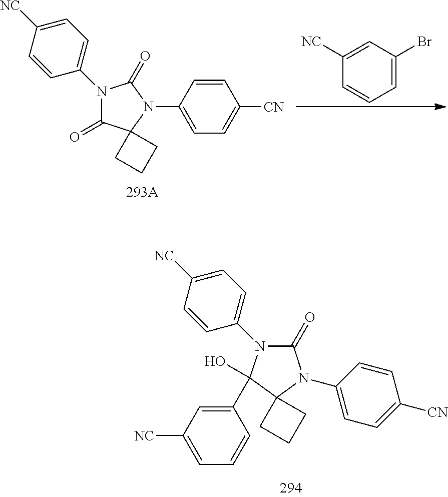 [SMILES: N#Cc1ccc(N2C(=O)N(c3ccc(C#N)cc3)C3(CCC3)C2=O)cc1 ; N#Cc1cccc(Br)c1 ; N#Cc1ccc(N2C(=O)N(c3ccc(C#N)cc3)C(O)(c3cccc(C#N)c3)C23CCC3)cc1]

Compound 294 was synthesized by employing the procedure described for Compound 125 using Compound 293A and 3-bromobenzonitrile in lieu of Compound 2C and 3-bromo-N,N-dimethylaniline. Compound 294. LC-MS (ESI) m/z: 446 [M+H]⁺; ¹H-NMR (400 MHz, CDCl₃): δ (ppm) 0.84-0.94 (m, 1H), 1.18-1.27 (m, 1H), 1.75-1.81 (m, 1H), 2.05-2.11 (m, 1H), 2.32-2.38 (m, 1H), 2.76-2.82 (m, 1H), 4.48 (bs, 1H), 7.43-7.58 (m, 7H), 7.66-7.68 (m, 2H), 7.75-7.77 (m, 2H), 7.94 (bs, 1H).

### Example 295

### Synthesis of 3-(7-(4-bromophenyl)-5-(4-cyanophenyl)-8-hydroxy-6-oxo-5,7-diazaspiro[3.4]octan-8-yl)benzonitrile

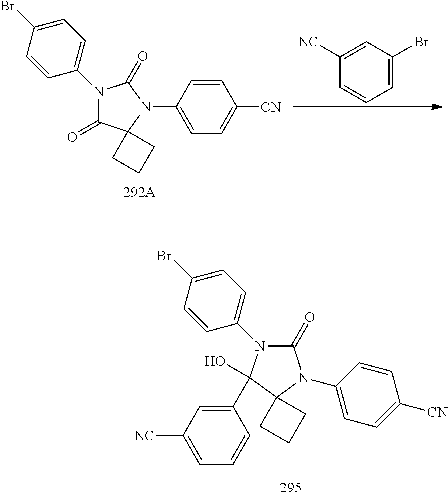 [SMILES: N#Cc1ccc(N2C(=O)N(c3ccc(Br)cc3)C(=O)C23CCC3)cc1 ; N#Cc1cccc(Br)c1 ; N#Cc1ccc(N2C(=O)N(c3ccc(Br)cc3)C(O)(c3cccc(C#N)c3)C23CCC3)cc1]

Compound 295 was synthesized by employing the procedure described for Compound 125 using Compound 292A and 3-bromobenzonitrile in lieu of Compound 2C and 3-bromo-N,N-dimethylaniline. Compound 295. LC-MS (ESI) m/z: 499 [M+H]⁺; ¹H-NMR (400 MHz, CDCl₃): δ (ppm) 0.79-0.88 (m, 1H), 1.14-1.22 (m, 1H), 1.72-1.78 (m, 1H), 2.00-2.06 (m, 1H), 2.27-2.33 (m, 1H), 2.72-2.78 (m, 1H), 4.40 (s, 1H), 7.25-7.27 (m, 2H), 7.30-7.33 (m, 2H), 7.43-7.46 (m, 3H), 7.62-7.64 (m, 2H), 7.69-7.71 (m, 2H), 7.92 (s, 1H).

### Example 296

### Synthesis of (4R,5R)-1,3-bis(4-chlorophenyl)-4-hydroxy-5-(methoxymethyl)-5-methyl-4-(3-(trifluoromethoxy)phenyl)imidazolidin-2-one, (4S,5S)-1,3-bis(4-chlorophenyl)-4-hydroxy-5-(methoxymethyl)-5-methyl-4-(3-(trifluoromethoxy)phenyl)imidazolidin-2-one, (4R,5S)-1,3-bis(4-chlorophenyl)-4-hydroxy-5-(methoxymethyl)-5-methyl-4-(3-(trifluoromethoxy)phenyl)imidazolidin-2-one, and (4S,5R)-1,3-bis(4-chlorophenyl)-4-hydroxy-5-(methoxymethyl)-5-methyl-4-(3-(trifluoromethoxy)phenyl)imidazolidin-2-one

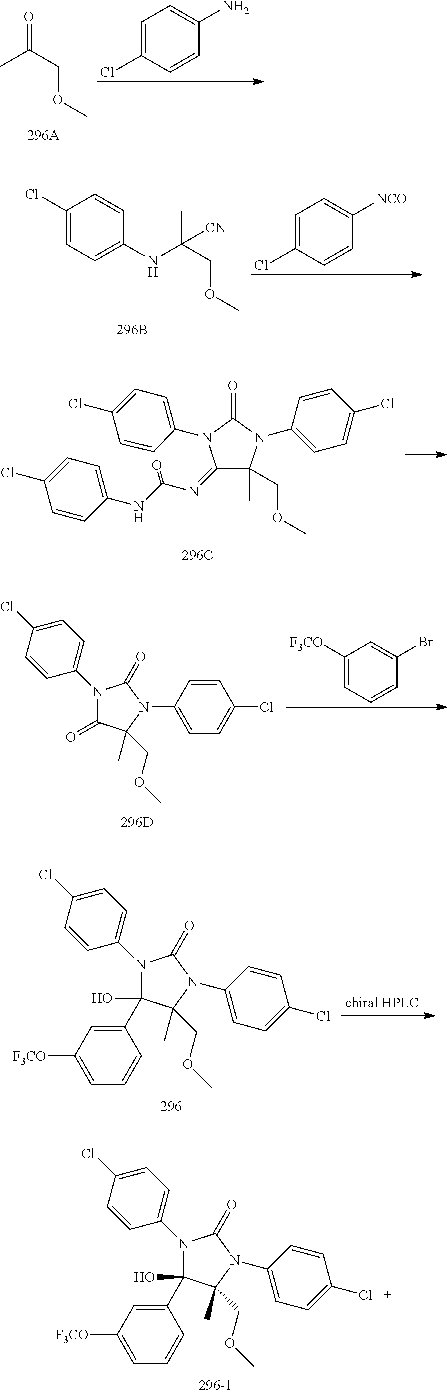

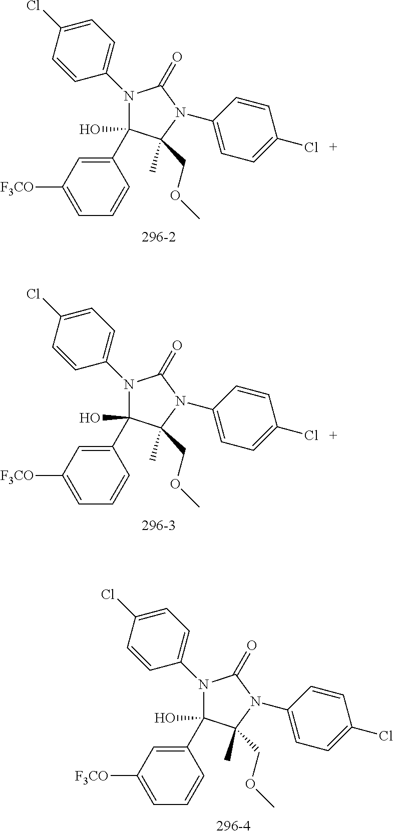 [SMILES: COC[C@]1(C)N(c2ccc(Cl)cc2)C(=O)N(c2ccc(Cl)cc2)[C@]1(O)c1cccc(OC(F)(F)F)c1 ; COC[C@]1(C)N(c2ccc(Cl)cc2)C(=O)N(c2ccc(Cl)cc2)[C@@]1(O)c1cccc(OC(F)(F)F)c1 ; COC[C@@]1(C)N(c2ccc(Cl)cc2)C(=O)N(c2ccc(Cl)cc2)[C@]1(O)c1cccc(OC(F)(F)F)c1]

Compounds 296B, 296C, 296D, and 296 were synthesized by employing the procedures described for Compounds 271B, 271C, 271D, and 125 using Compounds 296A, 296B, 296C with 3 M HCl solution, 296D, and 1-bromo-3-(trifluoromethoxy)benzene in lieu of Compounds 271A, 271B, 271C with 2 M H₂SO₄ solution, 2C, and 3-bromo-N,N-dimethylaniline. Compound 296B. LC-MS (ESI) m/z: 225 [M+H]⁺; ¹H-NMR (CDCl₃, 400 MHz): δ (ppm) 1.60 (s, 3H), 3.49 (s, 3H), 3.55 (d, J=9.2 Hz, 1H), 3.64 (d, J=9.2 Hz, 1H), 4.24 (s, 1H), 6.88 (d, J=8.8 Hz, 2H), 7.22 (d, J=8.8 Hz, 2H). Compound 296C. LC-MS (ESI) m/z: 531 [M+H]⁺. Compound 296D. LC-MS (ESI) m/z: 379 [M+H]⁺; ¹H-NMR (CDCl₃, 400 MHz): δ (ppm) 1.45 (s, 3H), 3.23 (d, J=10.0 Hz, 1H), 3.34 (s, 3H), 3.64 (d, J=9.6 Hz, 1H), 7.29 (d, J=8.8 Hz, 2H), 7.43-7.47 (m, 6H).

Compound 296. LC-MS (ESI) m/z: 541 [M+H]⁺; ¹H-NMR (CDCl₃, 400 MHz): δ (ppm) 0.65, 1.25 (s, 3H), 2.44, 3.19 (d, J=10.4 Hz, 1H), 2.79, 3.68 (d, J=10.4 Hz, 1H), 3.06, 3.48 (s, 3H), 3.62, 5.77 (s, 1H), 7.14-7.20 (m, 3H), 7.29-7.37 (m, 3H), 7.42-7.52 (m, 6H).

Compound 296 was separated with chiral preparative HPLC to yield Compounds 296-1, 296-2, 296-3, and 296-4. Compound 296-1. LC-MS (ESI) m/z: 541 [M+H]⁺; ¹H-NMR (CDCl₃, 400 MHz): δ (ppm) 1.22 (s, 3H), 2.41 (d, J=10.4 Hz, 1H), 2.76 (d, J=10.4 Hz, 1H), 3.04 (s, 3H), 4.07 (s, 1H), 7.10-7.15 (m, 3H), 7.28-7.44 (m, 9H); Chiral separation condition: MeOH contained 0.2% Methanol ammonia, Cellulose-SC (4.6*100 mm, 5 μm), retention time: 0.96 min. Compound 296-2. LC-MS (ESI) m/z: 541 [M+H]⁺; ¹H-NMR (CDCl₃, 500 MHz): δ (ppm) 1.25 (s, 3H), 2.45 (d, J=10.4 Hz, 1H), 2.80 (d, J=10.4 Hz, 1H), 3.06 (s, 3H), 3.68 (s, 1H), 7.12-7.16 (m, 3H), 7.30-7.44 (m, 9H); Chiral separation condition: MeOH contained 0.2% Methanol ammonia, Cellulose-SC (4.6*100 mm, 5 μm), retention time: 2.64 min. Compound 296-3. LC-MS (ESI) m/z: 541 [M+H]⁺; ¹H-NMR (CDCl₃, 400 MHz): δ (ppm) 0.68 (s, 3H), 3.20 (d, J=10.4 Hz, 1H), 3.48 (s, 3H), 3.68 (d, J=10.4 Hz, 1H), 5.76 (s, 1H), 7.12-7.20 (m, 3H), 7.25-7.28 (m, 3H), 7.40-7.46 (m, 6H); Chiral separation condition: MeOH contained 0.2% Methanol ammonia, OZ-H (4.6*100 mm, 5 μm), retention time: 0.82 min. Compound 296-4. LC-MS (ESI) m/z: 541 [M+H]⁺; ¹H-NMR (CDCl₃, 400 MHz): δ (ppm) 0.65 (s, 3H), 3.20 (d, J=10.4 Hz, 1H), 3.48 (s, 3H), 3.68 (d, J=10.4 Hz, 1H), 5.77 (s, 1H), 7.13-7.20 (m, 3H), 7.17-7.27 (m, 3H), 7.40-7.46 (m, 6H); Chiral separation condition: MeOH contained 0.2% Methanol ammonia, OZ-H (4.6*100 mm, 5 μm), retention time: 1.59 min.

### Example 297

### Synthesis of 3-((5R)-1,3-bis(4-chlorophenyl)-4-hydroxy-5-(methoxymethyl)-5-methyl-2-oxoimidazolidin-4-yl)benzonitrile and 3-((5S)-1,3-bis(4-chlorophenyl)-4-hydroxy-5-(methoxymethyl)-5-methyl-2-oxoimidazolidin-4-yl)benzonitrile

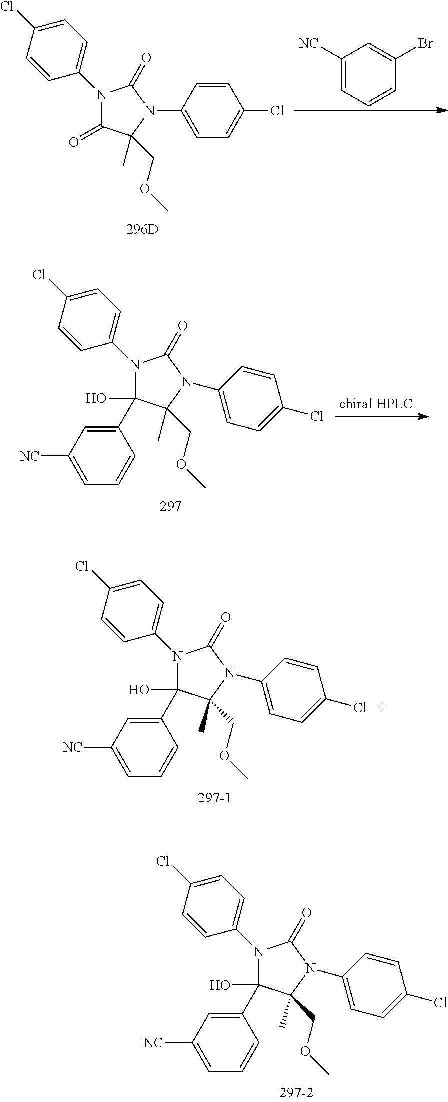 [SMILES: COCC1(C)C(=O)N(c2ccc(Cl)cc2)C(=O)N1c1ccc(Cl)cc1 ; N#Cc1cccc(Br)c1 ; COCC1(C)N(c2ccc(Cl)cc2)C(=O)N(c2ccc(Cl)cc2)C1(O)c1cccc(C#N)c1 ; COC[C@@]1(C)N(c2ccc(Cl)cc2)C(=O)N(c2ccc(Cl)cc2)C1(O)c1cccc(C#N)c1 ; COC[C@]1(C)N(c2ccc(Cl)cc2)C(=O)N(c2ccc(Cl)cc2)C1(O)c1cccc(C#N)c1]

Compound 297 was synthesized by employing the procedure described for Compound 125 using Compound 296D and 3-bromobenzonitrile in lieu of Compound 2C and 3-bromo-N,N-dimethylaniline. Compound 297. LC-MS (ESI) m/z: 482 [M+H]⁺; ¹H-NMR (CDCl₃, 400 MHz): δ (ppm) 0.64, 1.24 (s, 3H), 3.06, 3.49 (s, 3H), 3.20 (d, J=10.4 Hz, 1H), 3.68 (d, J=10.4 Hz, 1H), 5.88 (s, 1H), 7.15-7.18 (m, 2H), 7.23-7.30 (m, 2H), 7.38-7.46 (m, 5H), 7.62-7.70 (m, 3H).

Compound 297 was separated with chiral preparative HPLC to yield Compounds 297-1 and 297-2. Compound 297-1. LC-MS (ESI) m/z: 482 [M+H]⁺; ¹H-NMR (CDCl₃, 400 MHz): δ (ppm) 0.64 (s, 3H), 3.20 (d, J=10.4 Hz, 1H), 3.49 (s, 3H), 3.68 (d, J=10.4 Hz, 1H), 5.88 (s, 1H), 7.15-7.18 (m, 2H), 7.23-7.27 (m, 3H), 7.41-7.64 (m, 7H); Chiral separation condition: MeOH contained 0.2% Methanol ammonia, OZ-H (4.6*100 mm, 5 μm), retention time: 1.27 min. Compound 297-2. LC-MS (ESI) m/z: 482 [M+H]⁺; ¹H-NMR (CDCl₃, 400 MHz): δ (ppm) 0.64 (s, 3H), 3.20 (d, J=10.4 Hz, 1H), 3.49 (s, 3H), 3.68 (d, J=10.4 Hz, 1H), 5.89 (s, 1H), 7.14-7.17 (m, 2H), 7.23-7.27 (m, 3H), 7.41-7.64 (m, 7H); Chiral separation condition: MeOH contained 0.2% Methanol ammonia, OZ-H (4.6*100 mm, 5 μm), retention time: 2.46 min.

### Example 298

### Synthesis of 3-(5-(4-chlorophenyl)-8-hydroxy-6-oxo-7-(p-tolyl)-2-oxa-5,7-diazaspiro[3.4]octan-8-yl)benzonitrile

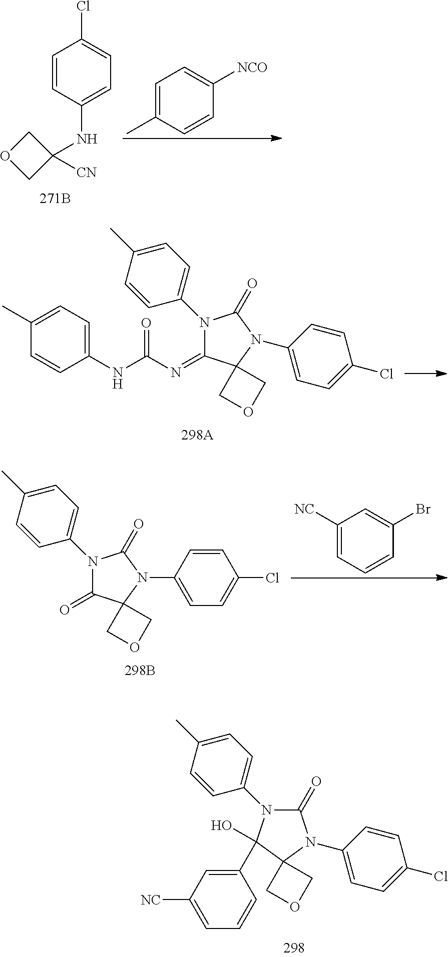

Compounds 298A, 298B, and 298 were synthesized by employing the procedures described for Compounds 271C, 271D, and 125 using 1-isocyanato-4-methylbenzene, Compounds 271B, 298A, 298B, and 3-bromobenzonitrile in lieu of 1-chloro-4-isocyanatobenzene, Compounds 271B, 271C, 2C, and 3-bromo-N,N-dimethylaniline. Compound 298A. LC-MS (ESI) m/z: 475 [M+H]⁺. Compound 298B. LC-MS (ESI) m/z: 343 [M+H]⁺; ¹H-NMR (DMSO-d₆, 400 MHz): δ (ppm) 2.44 (s, 3H), 4.96 (d, J=7.6 Hz, 2H), 5.21 (d, J=7.6 Hz, 2H), 7.33 (d, J=8.4 Hz, 2H), 7.37 (d, J=8.4 Hz, 2H), 7.57 (s, 4H). Compound 298. LC-MS (ESI) m/z: 446 [M+H]⁺; ¹H-NMR (CDCl₃, 400 MHz): δ (ppm) 2.30 (s, 3H), 3.93 (d, J=8.0 Hz, 1H), 4.46 (d, J=8.0 Hz, 1H), 4.66 (d, J=8.0 Hz, 1H), 4.70 (s, 1H), 5.15 (d, J=8.0 Hz, 1H), 7.05 (d, J=8.4 Hz, 2H), 7.14 (d, J=8.4 Hz, 2H), 7.21 (d, J=8.4 Hz, 2H), 7.37 (d, J=8.4 Hz, 2H), 7.48 (t, J=8.0 Hz, 1H), 7.64 (d, J=8.0 Hz, 2H), 7.87 (s, 1H).

### Example 299

### Synthesis of 5-(4-chlorophenyl)-8-hydroxy-7-(p-tolyl)-8-(3-(trifluoromethoxy)phenyl)-2-oxa-5,7-diazaspiro[3.4]octan-6-one

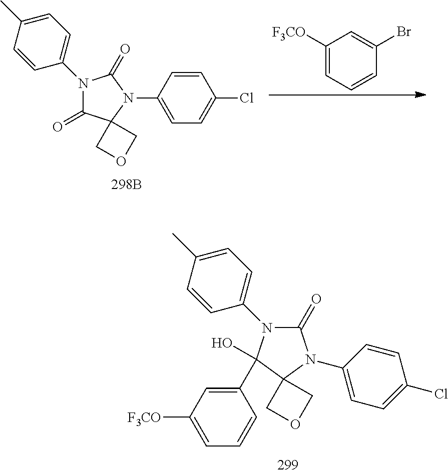 [SMILES: Cc1ccc(N2C(=O)N(c3ccc(Cl)cc3)C3(COC3)C2=O)cc1 ; FC(F)(F)Oc1cccc(Br)c1 ; Cc1ccc(N2C(=O)N(c3ccc(Cl)cc3)C3(COC3)C2(O)c2cccc(OC(F)(F)F)c2)cc1]

Compound 299 was synthesized by employing the procedure described for Compound 125 using Compound 298B and 3-bromobenzonitrile in lieu of Compound 2C and 3-bromo-N,N-dimethylaniline. Compound 299. LC-MS (ESI) m/z: 505 [M+H]⁺; ¹H-NMR (CDCl₃, 400 MHz): δ (ppm) 2.30 (s, 3H), 3.95 (d, J=8.0 Hz, 1H), 4.37 (d, J=8.0 Hz, 1H), 4.53 (d, J=8.4 Hz, 1H), 5.11 (d, J=8.0 Hz, 1H), 5.20 (s, 1H), 7.02 (d, J=8.4 Hz, 2H), 7.07-7.11 (m, 4H), 7.17 (d, J=7.2 Hz, 1H), 7.28-7.36 (m, 5H).

### Example 300

### Synthesis of 3-(5,7-bis(4-chlorophenyl)-8-hydroxy-6-oxo-2,5,7-triazaspiro[3.4]octan-8-yl)benzonitrile 2,2,2-trifluoroacetate

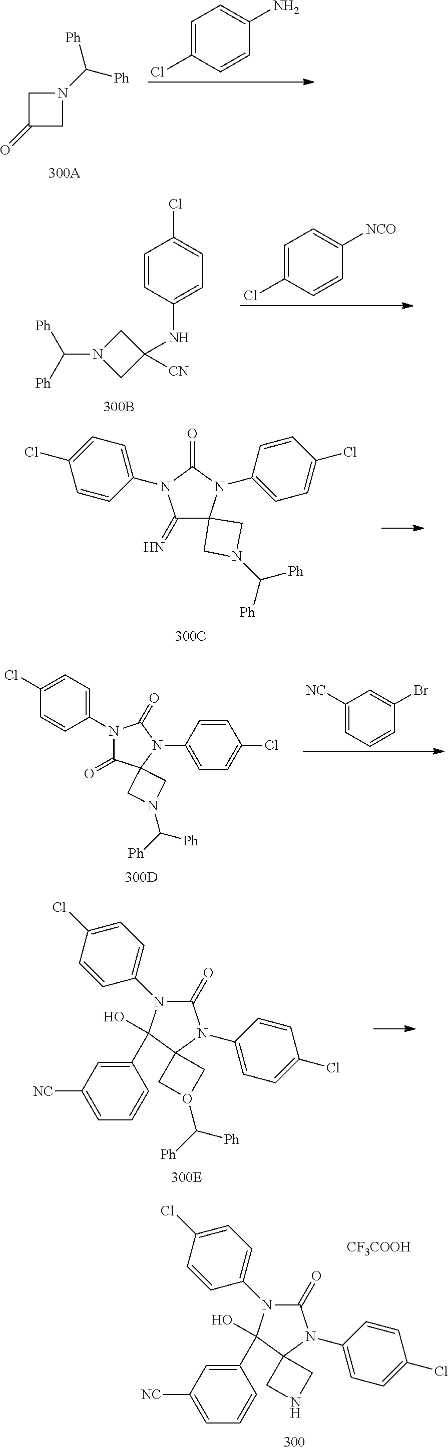

Compounds 300B, 300C, 300D, and 300E were synthesized by employing the procedures described for Compounds 271B, 271C, 271D, and 125 using Compounds 300A, 300B, 300C, 300D, and 3-bromobenzonitrile in lieu of Compounds 271A, 271B, 271C, 2C, and 3-bromo-N,N-dimethylaniline. Compound 300B. LC-MS (ESI) m/z: 374 [M+H]⁺; ¹H-NMR (CDCl₃, 400 MHz): δ (ppm) 3.21-3.34 (m, 2H), 3.69-3.83 (m, 2H), 3.39, 4.48 (s, 1H), 6.51 (d, J=8.8 Hz, 1H), 7.16-7.24 (m, 4H), 7.26-7.31 (m, 6H), 7.41-7.43 (m, 4H). Compound 300C. LC-MS (ESI) m/z: 527 [M+H]⁺; Compound 300D. LC-MS (ESI) m/z: 528 [M+H]⁺; ¹H-NMR (CDCl₃, 400 MHz): δ (ppm) 3.57-3.73 (m, 4H), 4.51 (s, 1H), 7.18-7.26 (m, 8H), 7.26-7.30 (m, 2H), 7.41-7.56 (m, 5H), 7.59-7.68 (m, 3H). Compound 300E. LC-MS (ESI) m/z: 631 [M+H]⁺; ¹H-NMR (CDCl₃, 500 MHz): δ (ppm) 2.46-2.48 (m, 1H), 2.73-7.75 (m, 1H), 3.09-3.11 (m, 1H), 3.64-3.66 (m, 1H), 3.76 (s, 1H), 4.60 (s, 1H), 6.70-6.72 (m, 2H), 6.95-6.97 (m, 2H), 7.01-7.26 (m, 12H), 7.38-7.40 (m, 2H), 7.45-7.46 (m, 1H), 7.57 (s, 1H), 7.68-7.70 (m, 1H), 7.95 (s, 1H).

A mixture of Compound 300E (800 mg, 1.3 mmol) and CAN (3.5 g, 6.5 mmol) in MeCN (50 mL) and water (10 mL) was stirred at room temperature for 3 hours. The reaction mixture was quenched with a saturated NaHCO₃ solution (100 mL) and extracted with ethyl acetate (50 mL×3). The combined organic layers was washed with brine (50 mL), dried over anhydrous sodium sulfate, filtered, and concentrated. The residue was purified with preparative HPLC to yield Compound 300. LC-MS (ESI) m/z: 465 [M+H]⁺; ¹H-NMR (DMSO-d₆, 400 MHz): δ (ppm) 3.22-3.24 (m, 1H), 3.90-4.04 (m, 2H), 4.60-4.63 (m, 1H), 7.29-7.32 (m, 2H), 7.41-7.43 (m, 2H), 7.64-7.72 (m, 5H), 7.87-7.89 (m, 1H), 8.08 (s, 1H), 8.26-8.30 (m, 2H), 8.47 (s, 1H).

### Example 301

### Synthesis of 3-(5,7-bis(4-chlorophenyl)-8-hydroxy-2-methyl-6-oxo-2,5,7-triazaspiro[3.4]octan-8-yl)benzonitrile 2,2,2-trifluoroacetate

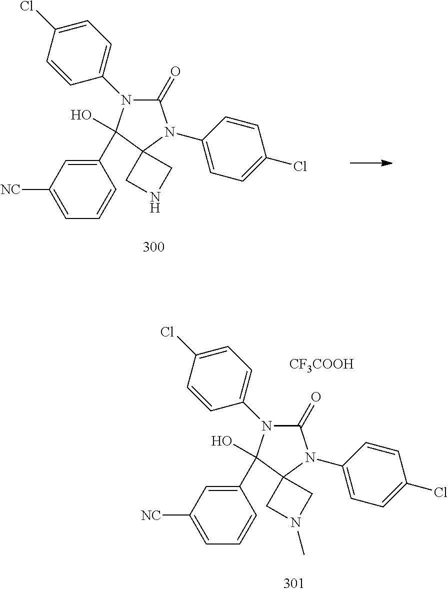 [SMILES: N#Cc1cccc(C2(O)N(c3ccc(Cl)cc3)C(=O)N(c3ccc(Cl)cc3)C23CNC3)c1 ; CN1CC2(C1)N(c1ccc(Cl)cc1)C(=O)N(c1ccc(Cl)cc1)C2(O)c1cccc(C#N)c1 ; "CF3COOH"]

To a solution of Compound 300 (200 mg, 0.34 mmol) in MeOH (5 mL) was added paraldehyde (51 mg, 1.7 mmol) and stirred at 60° C. for 1 hour. After cooled down to room temperature, to the mixture was added NaBH₃CN (109 mg, 1.7 mmol) in one portion and stirred at room temperature for 3 hours. The reaction mixture was quenched with saturated NH₄Cl solution (50 mL) and extracted with ethyl acetate (50 mL×3). The combined organic layers was washed with brine (50 mL), dried over anhydrous sodium sulfate, filtered, and concentrated. The residue was purified with preparative HPLC to yield Compound 301. LC-MS (ESI) m/z: 479 [M+H]⁺; ¹H-NMR (DMSO-d₆, 400 MHz): δ (ppm) 2.01, 2.12 (s, 3H), 3.04-3.90 (m, 2H), 4.27-4.40 (m, 1H), 4.64-5.00 (m, 1H), 7.27-7.30 (m, 2H), 7.39-7.42 (m, 2H), 7.65-7.75 (m, 5H), 7.86-8.33 (m, 3H), 7.80 (s, 1H).

### Example 302

### Synthesis of 8-hydroxy-8-(3-(trifluoromethoxy)phenyl)-5,7-bis(4-(trifluoromethyl)phenyl)-2-oxa-5,7-diazaspiro[3.4]octan-6-one

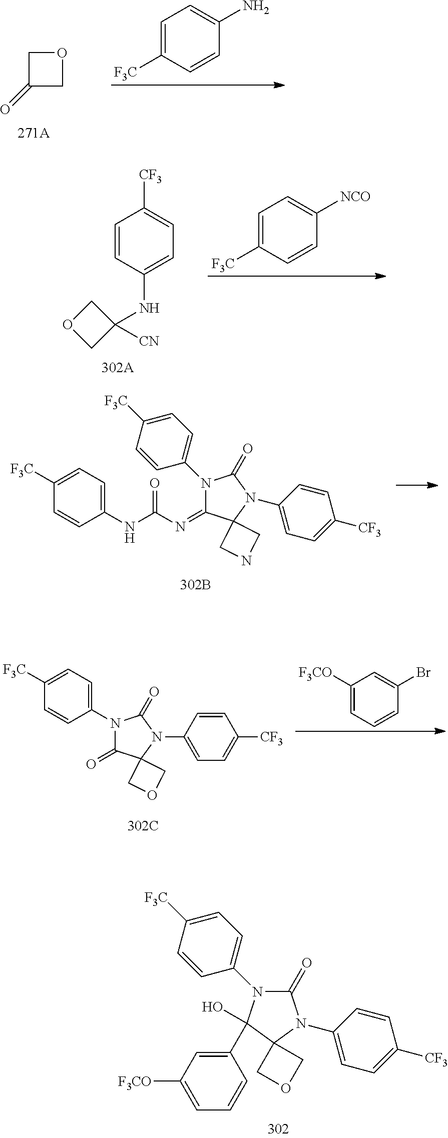

Compounds 302A, 302B, 302C, and 302 were synthesized by employing the procedures described for Compounds 271B, 271C, 271D, and 125 using 4-(trifluoromethyl)aniline, 1-isocyanato-4-(trifluoromethyl)benzene, Compounds 302A, 302B, 302C, and 1-bromo-3-(trifluoromethoxy)benzene in lieu of 4-chloroaniline, 1-chloro-4-isocyanatobenzene, Compounds 271B, 271C, 2C, and 3-bromo-N,N-dimethylaniline. Compound 302A. LC-MS (ESI) m/z: 243 [M+H]⁺; ¹H-NMR (CDCl₃, 400 MHz): δ (ppm) 4.68 (s, 1H), 4.73 (d, J=6.4 Hz, 2H), 5.17 (d, J=6.4 Hz, 2H), 6.58 (d, J=8.8 Hz, 2H), 7.53 (d, J=8.8 Hz, 2H). Compound 302B. LC-MS (ESI) m/z: 617 [M+H]⁺. Compound 302C. LC-MS (ESI) m/z: 431 [M+H]⁺; ¹H-NMR (DMSO-d₆, 400 MHz): δ (ppm) 4.77 (d, J=8.4 Hz, 2H), 4.90 (d, J=8.4 Hz, 2H), 7.73 (d, J=8.8 Hz, 2H), 7.93-7.97 (m, 6H). Compound 302. LC-MS (ESI) m/z: 593 [M+H]⁺; ¹H-NMR (CD₃OD, 400 MHz): δ (ppm) 4.07 (d, J=8.0 Hz, 1H), 4.62 (d, J=8.4 Hz, 1H), 4.77 (d, J=8.8 Hz, 1H), 5.29 (d, J=8.4 Hz, 1H), 7.29-7.31 (m, 1H), 7.50-7.52 (m, 4H), 7.53-7.62 (m, 1H), 7.71-7.73 (m, 2H), 7.89 (s, 4H).

### Example 303

### Synthesis of 3-(8-hydroxy-6-oxo-5,7-bis(4-(trifluoromethyl)phenyl)-2-oxa-5,7-diazaspiro[3.4]octan-8-yl)benzonitrile

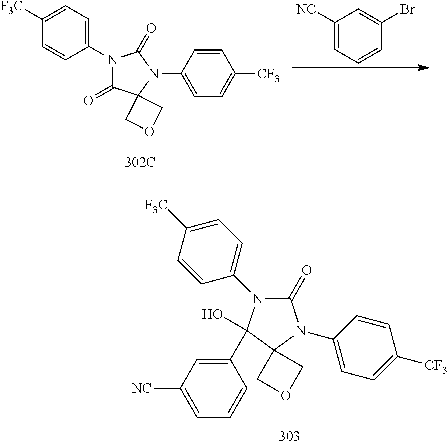 [SMILES: O=C1N(c2ccc(C(F)(F)F)cc2)C(=O)C2(COC2)N1c1ccc(C(F)(F)F)cc1 ; N#Cc1cccc(Br)c1 ; N#Cc1cccc(C2(O)N(c3ccc(C(F)(F)F)cc3)C(=O)N(c3ccc(C(F)(F)F)cc3)C23COC3)c1]

Compound 303 was synthesized by employing the procedure described for Compound 125 using Compound 302C and 3-bromobenzonitrile in lieu of Compound 2C and 3-bromo-N,N-dimethylaniline. Compound 303. LC-MS (ESI) m/z: 534 [M+H]⁺; ¹H-NMR (CD₃OD, 400 MHz): δ (ppm) 4.01 (d, J=8.4 Hz, 1H), 4.63 (d, J=8.4 Hz, 1H), 4.78 (d, J=8.8 Hz, 1H), 5.30 (d, J=8.4 Hz, 1H), 7.52-7.58 (m, 3H), 7.73-7.75 (m, 3H), 7.89 (s, 5H), 8.11 (s, 1H).

### Example 304

### Synthesis of 5,7-bis(4-chlorophenyl)-2,2-difluoro-8-hydroxy-8-(3-(trifluoromethoxy)phenyl)-5,7-diazaspiro[3.4]octan-6-one

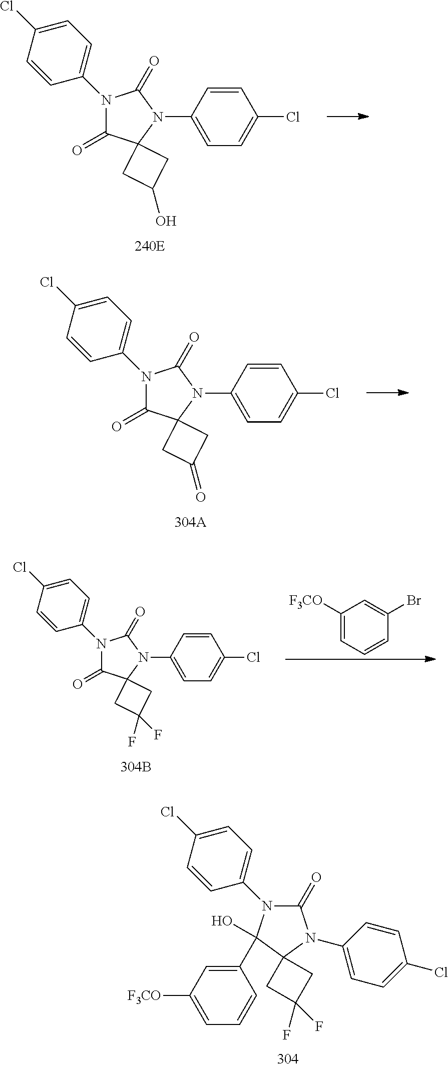 [SMILES: O=C1N(c2ccc(Cl)cc2)C(=O)C2(CC(O)C2)N1c1ccc(Cl)cc1 ; O=C1CC2(C1)C(=O)N(c1ccc(Cl)cc1)C(=O)N2c1ccc(Cl)cc1 ; O=C1N(c2ccc(Cl)cc2)C(=O)C2(CC(F)(F)C2)N1c1ccc(Cl)cc1 ; FC(F)(F)Oc1cccc(Br)c1 ; O=C1N(c2ccc(Cl)cc2)C2(CC(F)(F)C2)C(O)(c2cccc(OC(F)(F)F)c2)N1c1ccc(Cl)cc1]

To a solution of Compound 240E (377 mg, 1.0 mmol) in dichloromethane (5 mL) was added DMP (636 mg, 1.5 mmol) and stirred at room temperature overnight. The mixture was washed with saturated sodium hydrogen sulfite solution (5 mL), dried over anhydrous sodium sulfate, filtered, and concentrated. The residue was purified by flash column chromatography on silica gel (ethyl acetate in petroleum ether, 20% v/v) to yield Compound 304A. LC-MS (ESI) m/z: 375 [M+H]⁺; ¹H-NMR (400 MHz, CDCl₃): δ (ppm) 3.47-3.52 (m, 2H), 3.79-3.84 (m, 2H), 7.31-7.33 (m, 2H), 7.48-7.49 (m, 4H), 7.51-7.53 (m, 2H).

The mixture of Compound 304A (750 mg, 2.0 mmol) and bis(2-methoxyethyl)amino-sulfur trifluoride (5 mL) was stirred at 60° C. for 5 hours. After cooled down to room temperature, the mixture was dropped into ice-water (20 mL), and extracted with ethyl acetate (10 mL×2). The combined organic layers was dried over anhydrous sodium sulfate, filtered, and concentrated. The crude product was purified by flash column chromatography on silica gel (ethyl acetate in petroleum ether, 20% v/v) to afford Compound 304B. LC-MS (ESI) m/z: 397 [M+H]⁺; ¹H-NMR (400 MHz, CDCl₃): δ (ppm) 3.03-3.10 (m, 2H), 3.37-3.44 (m, 2H), 7.28-7.31 (m, 2H), 7.45-7.48 (m, 4H), 7.51-7.54 (m, 2H).

Compound 304 was synthesized by employing the procedure described for Compound 125 using Compound 304B and 1-bromo-3-(trifluoromethoxy)benzene in lieu of Compound 2C and 3-bromo-N,N-dimethylaniline. Compound 304. LC-MS (ESI) m/z: 559 [M+H]⁺; ¹H-NMR (400 MHz, CDCl₃): δ (ppm) 2.36-2.41 (m, 1H), 2.57-2.63 (m, 1H), 2.83-2.86 (m, 1H), 3.40-3.47 (m, 1H), 4.11 (br, 1H), 7.18-7.26 (m, 6H), 7.29-7.32 (m, 2H), 7.35-7.40 (m, 2H), 7.42-7.44 (m, 2H).

### Example 305

### Synthesis of 3-(5,7-bis(4-chlorophenyl)-2,2-difluoro-8-hydroxy-6-oxo-5,7-diazaspiro[3.4]octan-8-yl)benzonitrile

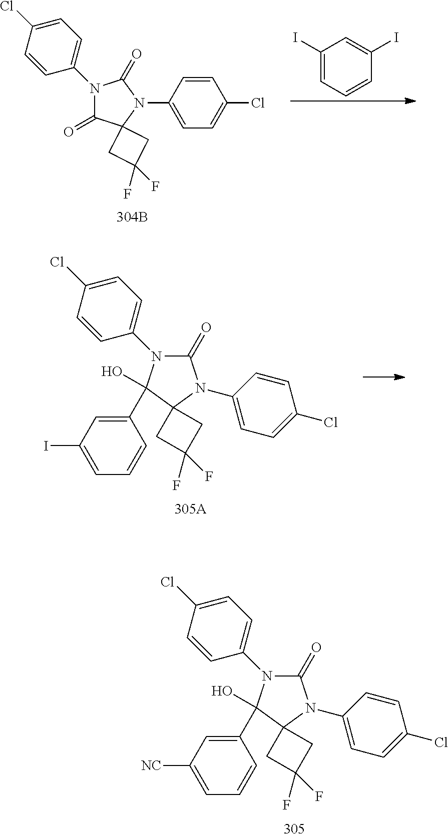 [SMILES: O=C1N(c2ccc(Cl)cc2)C(=O)C2(CC(F)(F)C2)N1c1ccc(Cl)cc1 ; Ic1cccc(I)c1 ; O=C1N(c2ccc(Cl)cc2)C2(CC(F)(F)C2)C(O)(c2cccc(I)c2)N1c1ccc(Cl)cc1 ; N#Cc1cccc(C2(O)N(c3ccc(Cl)cc3)C(=O)N(c3ccc(Cl)cc3)C23CC(F)(F)C3)c1]

Compounds 305A and 305 were synthesized by employing the procedures described for Compounds 125 and 180C using 1,3-diiodobenzene, Compounds 304B, and 305A in lieu of 3-bromo-N,N-dimethylaniline, Compounds 2C, and 180B. Compound 305A. LC-MS (ESI) m/z: 601 [M+H]⁺; ¹H-NMR (400 MHz, CDCl₃): δ (ppm) 2.34-2.37 (m, 1H), 2.74-2.78 (m, 1H), 2.88-2.92 (m, 1H), 3.48-3.52 (m, 1H), 7.12-7.16 (m, 1H), 7.22-7.26 (m, 2H), 7.48-7.52 (m, 5H), 7.55-7.58 (m, 2H), 7.71-7.73 (m, 1H), 7.98 (br, 1H). Compound 305. LC-MS (ESI) m/z: 500 [M+H]⁺; ¹H-NMR (400 MHz, CDCl₃): δ (ppm) 2.16-2.26 (m, 1H), 2.50-2.57 (m, 1H), 2.72-2.80 (m, 1H), 3.32-3.40 (m, 1H), 5.18 (s, 1H), 7.05-7.07 (m, 2H), 7.16-7.21 (m, 4H), 7.33-7.35 (m, 2H), 7.42-7.52 (m, 2H), 7.61-7.78 (m, 2H).

### Example 306

### Synthesis of 5-(5,7-bis(4-chlorophenyl)-8-hydroxy-6-oxo-2-oxa-5,7-diazaspiro[3.4]octan-8-yl)isophthalonitrile

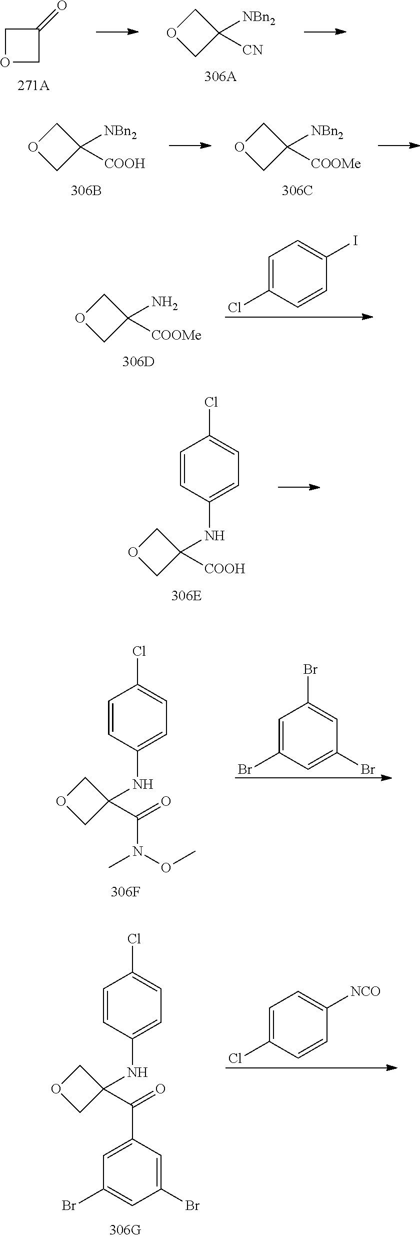

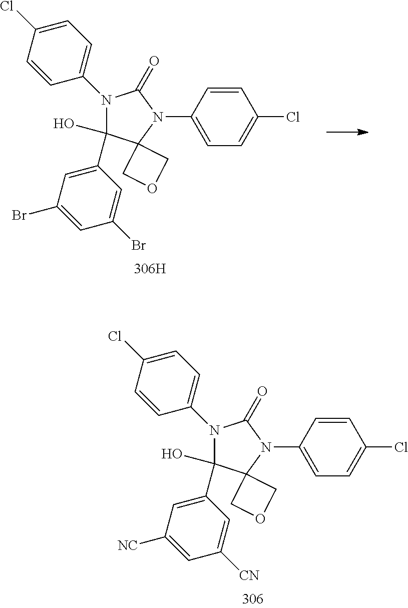 [SMILES: O=C1N(c2ccc(Cl)cc2)C2(COC2)C(O)(c2cc(Br)cc(Br)c2)N1c1ccc(Cl)cc1 ; N#Cc1cc(C#N)cc(C2(O)N(c3ccc(Cl)cc3)C(=O)N(c3ccc(Cl)cc3)C23COC3)c1]

To a stirred solution of oxetan-3-one 271A (15.6 g, 216.7 mmol) in AcOH (100 mL) was added dibenzylamine (42.7 g, 216.7 mmol) at 0° C. After the mixture was stirred at room temperature for 1 hour, to it was added TMSCN (21.5 g, 216.7 mmol) and stirred at room temperature overnight. The mixture was concentrated under the reduced pressure. The residue was partitioned between water (100 mL) and ethyl acetate (500 mL). The organic was separated and the aqueous was extracted with ethyl acetate (100 mL×2). The combined organic layers was washed with saturated NaHCO₃ solution (300 mL×2) and brine (300 mL×2), dried over anhydrous sodium sulfate, filtered, and concentrated to give a crude product, which was purified with flash column chromatography on silica gel (ethyl acetate in petroleum ether, 3% v/v) to afford Compound 306A. LC-MS (ESI) m/z: 279 [M+H]⁺; ¹H-NMR (CDCl₃, 400 MHz): δ (ppm) 3.52 (s, 4H), 4.30 (d, J=6.8 Hz, 2H), 4.34 (d, J=6.8 Hz, 2H), 7.33-7.41 (m, 10H).

To a solution of Compound 306A (35 g, 125.7 mmol) in ethanol (300 mL) was added a solution of NaOH (20.1 g, 502.8 mmol) in water (200 mL) and heated at reflux overnight. To the mixture was added another portion of NaOH (20 g, 251.4 mmol), stirred at reflux for 48 hours more, and evaporated to remove most of ethanol. The residue was diluted with water (200 mL) and washed with ethyl ether (100 mL×2). The aqueous layer was adjusted to pH 4 with concentrated HCl at 0° C. and precipitated a white solid. The solid was collected by filtration, washed with water (100 mL×2), and dried under vacuum to furnish Compound 306B. LC-MS (ESI) m/z: 298 [M+H]⁺; ¹H-NMR (CDCl₃, 500 MHz): δ (ppm) 3.69 (s, 4H), 4.44 (d, J=6.5 Hz, 2H), 4.56 (d, J=6.5 Hz, 2H), 7.26-7.27 (m, 2H), 7.30-7.32 (m, 4H), 7.37-7.39 (m, 4H).

Compound 306C was synthesized by employing the procedure described for Compound 268B using Compounds 306B in lieu of Compound 268A. Compound 306C. LC-MS (ESI) m/z: 312 [M+H]⁺; ¹H-NMR (CDCl₃, 400 MHz): δ (ppm) 3.61 (s, 4H), 3.93 (s, 3H), 4.40 (d, J=6.8 Hz, 2H), 4.51 (d, J=6.4 Hz, 2H), 7.23-7.37 (m, 10H).

To a mixture of Compound 306C (20 g, 64.3 mmol) in methanol (100 mL) and ethyl acetate (100 mL) was added Pd/C (10%, 2 g), stirred under hydrogen (1 atm.) at room temperature overnight, and filtered. The filtrate was concentrated to give a crude product Compound 306D. LC-MS (ESI) m/z: 132 [M+H]⁺; ¹H-NMR (CDCl₃, 400 MHz): δ (ppm) 2.08 (s, 2H), 3.88 (s, 3H), 4.53 (d, J=6.4 Hz, 2H), 5.00 (d, J=7.6 Hz, 2H).

Compounds 306E, 306F, 306G, 306H, and 306 were synthesized by employing the procedures described for Compounds 224B, 27H, 27, 12, and 180C using Compounds 306D with 2-acetylcyclohexan-1-one, 306E, 306F, 1,3,5-tribromobenzene with n-butyl lithium as base, and 306H in lieu of Compounds 224A without 2-acetylcyclohexan-1-one, 27G, 27H, phenyllithium, 12B, and 180B. Compound 306E. LC-MS (ESI) m/z: 228 [M+H]⁺. Compound 306F. LC-MS (ESI) m/z: 271 [M+H]⁺; ¹H-NMR (CDCl₃, 400 MHz): 3.17 (s, 3H), 3.65 (s, 3H), 4.50 (s, 1H), 4.54 (d, J=7.2 Hz, 2H), 5.15 (d, J=7.2 Hz, 2H), 6.44 (d, J=6.8 Hz, 2H), 7.10 (d, J=10.4 Hz, 2H). Compound 306G. LC-MS (ESI) m/z: 444 [M+H]⁺; ¹H-NMR (CDCl₃, 400 MHz): 4.71 (s, 1H), 4.74 (d, J=6.8 Hz, 2H), 5.30 (d, J=7.2 Hz, 2H), 6.31-6.35 (m, 2H), 7.06-7.10 (m, 2H), 7.81 (t, J=1.6 Hz, 1H), 7.92 (d, J=1.6 Hz, 2H). Compound 306H. LC-MS (ESI) m/z: 597 [M+H]⁺; ¹H-NMR (CDCl₃, 400 MHz): 3.99 (s, 1H), 4.09 (d, J=8.0 Hz, 1H), 4.50 (d, J=7.6 Hz, 1H), 4.69 (d, J=8.8 Hz, 1H), 5.16 (d, J=8.0 Hz, 1H), 7.22-7.24 (m, 2H), 7.27-7.34 (m, 4H), 7.45 (d, J=8.8 Hz, 2H), 7.57 (s, 2H), 7.68 (t, J=2.0 Hz, 1H). Compound 306. LC-MS (ESI) m/z: 491 [M+H]⁺; ¹H-NMR (400 MHz, DMSO-d₆): δ (ppm) 3.71 (d, J=8.4 Hz, 1H), 4.40 (d, J=8.4 Hz, 1H), 4.55 (d, J=8.4 Hz, 1H), 5.14 (d, J=8.4 Hz, 1H), 7.30-7.33 (m, 2H), 7.44-7.46 (m, 2H), 7.65 (s, 4H), 8.20 (s, 1H), 8.40 (brs, 2H), 8.46 (t, J=1.2 Hz, 1H).

### Example 307

### Synthesis of 4-(8-hydroxy-6-oxo-5-(p-tolyl)-8-(3-(trifluoromethoxy)phenyl)-2-oxa-5,7-diazaspiro[3.4]octan-7-yl)benzonitrile

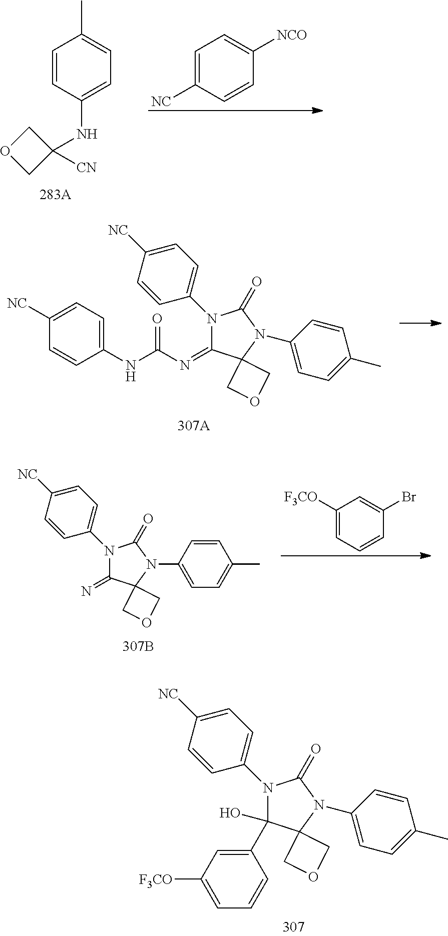

Compounds 307A, 307B, and 307 were synthesized by employing the procedures described for Compounds 271C, 271D, and 125 using 4-isocyanatobenzonitrile, Compounds 283A, 307A, 307B, and 1-bromo-3-(trifluoromethoxy)benzene in lieu of 1-chloro-4-isocyanatobenzene, Compounds 271B, 271C, 2C, and 3-bromo-N,N-dimethylaniline. Compound 307A. LC-MS (ESI) m/z: 477 [M+H]⁺. Compound 307B. LC-MS (ESI) m/z: 334 [M+H]⁺. Compound 307. LC-MS (ESI) m/z: 496 [M+H]⁺; ¹H-NMR (CDCl₃, 400 MHz): δ (ppm) 2.44 (s, 3H), 3.98 (d, J=8.0 Hz, 1H), 4.50 (d, J=7.6 Hz, 1H), 4.64 (s, 1H), 4.69 (d, J=8.4 Hz, 1H), 5.15 (d, J=8.0 Hz, 1H), 7.20 (d, J=8.4 Hz, 2H), 7.24-7.30 (m, 5H), 7.41-7.43 (m, 3H), 7.51 (d, J=8.8 Hz, 2H).

### Example 308

### Synthesis of 3-(7-(4-cyanophenyl)-8-hydroxy-6-oxo-5-(p-tolyl)-2-oxa-5,7-diazaspiro[3.4]octan-8-yl)benzonitrile

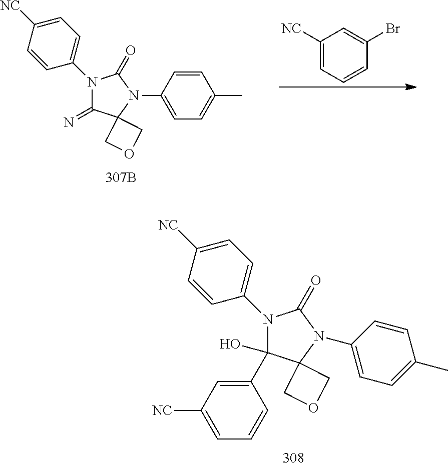 [SMILES: Cc1ccc(N2C(=O)N(c3ccc(C#N)cc3)C(=N)C23COC3)cc1 ; N#Cc1cccc(Br)c1 ; Cc1ccc(N2C(=O)N(c3ccc(C#N)cc3)C(O)(c3cccc(C#N)c3)C23COC3)cc1]

Compound 308 was synthesized by employing the procedure described for Compound 125 using Compound 307B and 3-bromobenzonitrile in lieu of Compound 2C and 3-bromo-N,N-dimethylaniline. Compound 308. LC-MS (ESI) m/z: 437 [M+H]⁺; ¹H-NMR (CDCl₃, 400 MHz): δ (ppm) 2.43 (s, 3H), 3.85 (d, J=7.6 Hz, 1H), 4.50 (d, J=7.6 Hz, 1H), 4.68 (d, J=8.4 Hz, 1H), 5.11 (d, J=7.6 Hz, 1H), 5.40 (s, 1H), 7.19 (d, J=8.0 Hz, 2H), 7.26-7.28 (m, 3H), 7.41 (d, J=8.8 Hz, 2H), 7.47-7.53 (m, 3H), 7.67 (d, J=7.6 Hz, 1H), 7.90 (s, 1H).

### Example 309

### Synthesis of 3-(3-(4-chlorophenyl)-1-(4-cyanophenyl)-4-hydroxy-5,5-dimethyl-2-oxoimidazolidin-4-yl)benzonitrile

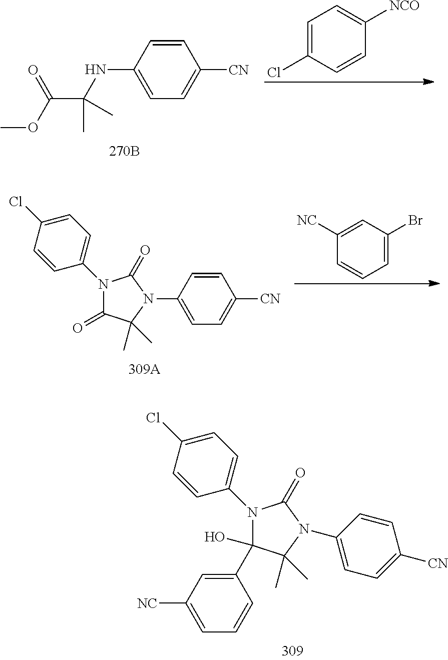

Compounds 309A and 309 were synthesized by employing the procedures described for Compounds 224D and 125 using Compounds 270B, 309A, and 3-bromobenzonitrile in lieu of Compounds 224C, 2C and 3-bromo-N,N-dimethylaniline. Compound 309A. LC-MS (ESI) m/z: 340 [M+H]⁺; ¹H-NMR (CDCl₃, 400 MHz): δ (ppm) 1.62 (s, 6H), 7.44-7.48 (m, 4H), 7.52-7.54 (m, 2H), 7.76-7.79 (m, 2H). Compound 309. LC-MS (ESI) m/z: 443 [M+H]⁺. ¹H-NMR (CDCl₃, 400 MHz): δ (ppm) 0.81 (s, 3H), 1.37 (s, 3H), 4.58 (s, 1H), 7.17-7.19 (m, 2H), 7.33-7.36 (m, 4H), 7.38-7.41 (m, 1H), 7.54-7.61 (m, 2H), 7.65-7.66 (m, 2H), 7.78 (s, 1H).

### Example 310

### Synthesis of 8-(3-bromothiophen-2-yl)-5,7-bis(4-chlorophenyl)-8-hydroxy-2-oxa-5,7-diazaspiro[3.4]octan-6-one

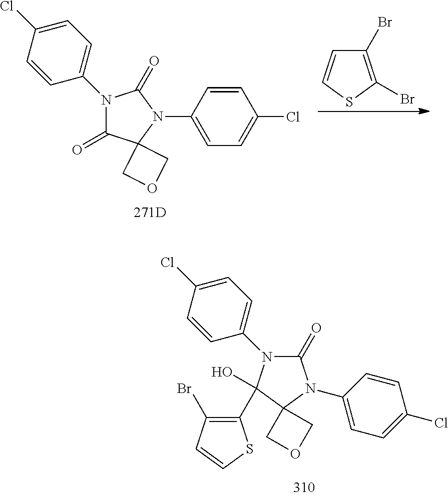

Compound 310 was synthesized by employing the procedure described for Compound 125 using Compound 271D and 2,3-dibromothiophene in lieu of Compound 2C and 3-bromo-N,N-dimethylaniline. Compound 310. LC-MS (ESI) m/z: 525 [M+H]⁺; ¹H-NMR (CDCl₃, 400 MHz): δ (ppm) 4.27 (d, J=7.6 Hz, 1H), 4.50 (d, J=7.6 Hz, 1H), 4.63 (d, J=8.4 Hz, 1H), 4.94 (s, 1H), 5.19 (d, J=8.4 Hz, 1H), 6.89 (d, J=1.2 Hz, 1H), 7.21-7.29 (m, 7H), 7.41 (d, J=8.8 Hz, 2H).

### Example 311

### Synthesis of 7-(4-chlorophenyl)-8-hydroxy-5-(p-tolyl)-8-(3-(trifluoromethoxy)phenyl)-2-oxa-5,7-diazaspiro[3.4]octan-6-one

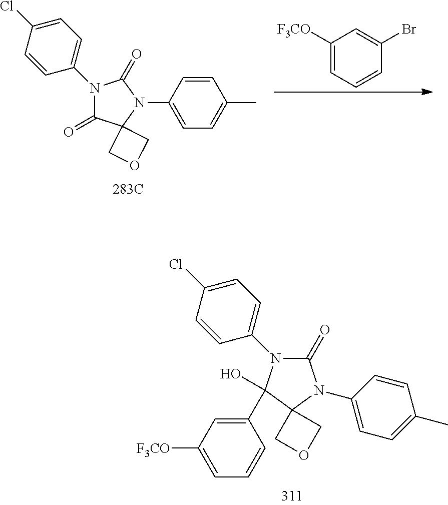 [SMILES: Cc1ccc(N2C(=O)N(c3ccc(Cl)cc3)C(=O)C23COC3)cc1 ; FC(F)(F)Oc1cccc(Br)c1 ; Cc1ccc(N2C(=O)N(c3ccc(Cl)cc3)C(O)(c3cccc(OC(F)(F)F)c3)C23COC3)cc1]

Compound 311 was synthesized by employing the procedure described for Compound 125 using Compound 283C and 1-bromo-3-(trifluoromethoxy)benzene in lieu of Compound 2C and 3-bromo-N,N-dimethylaniline. Compound 311. LC-MS (ESI) m/z: 505 [M+H]⁺; ¹H-NMR (CDCl₃, 400 MHz): δ (ppm) 2.43 (s, 3H), 3.99 (d, J=7.6 Hz, 1H), 4.49 (d, J=7.6 Hz, 1H), 4.68 (d, J=8.0 Hz, 1H), 4.78 (s, 1H), 5.15 (d, J=8.4 Hz, 1H), 7.15-7.18 (m, 4H), 7.22 (d, J=8.0 Hz, 1H), 7.26 (d, J=8.0 Hz, 2H), 7.30 (d, J=9.2 Hz, 2H), 7.36 (s, 1H), 7.39-7.41 (m, 2H).

### Example 312

### Synthesis of 4-(4-hydroxy-5,5-dimethyl-2-oxo-3-(p-tolyl)-4-(3-(trifluoromethoxy)phenyl)imidazolidin-1-yl)benzonitrile

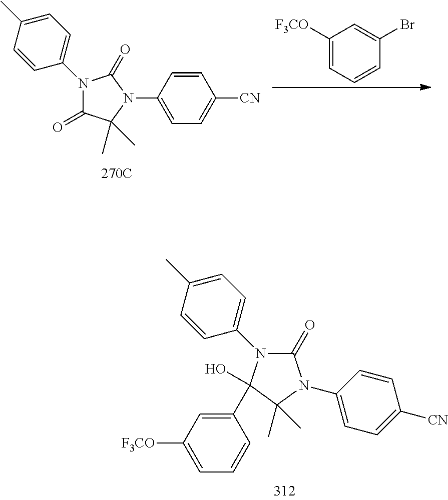 [SMILES: Cc1ccc(N2C(=O)N(c3ccc(C#N)cc3)C(C)(C)C2=O)cc1 ; FC(F)(F)Oc1cccc(Br)c1 ; Cc1ccc(N2C(=O)N(c3ccc(C#N)cc3)C(C)(C)C2(O)c2cccc(OC(F)(F)F)c2)cc1]

Compound 312 was synthesized by employing the procedure described for Compound 125 using Compound 270C and 1-bromo-3-(trifluoromethoxy)benzene in lieu of Compound 2C and 3-bromo-N,N-dimethylaniline. Compound 312. LC-MS (ESI) m/z: 482 [M+H]⁺; ¹H-NMR (CDCl₃, 400 MHz): δ (ppm) 0.83 (s, 3H), 1.37 (s, 3H), 2.28 (s, 3H), 4.17 (s, 1H), 7.04 (d, J=6.8 Hz, 2H), 7.15 (d, J=6.4 Hz, 1H), 7.26-7.37 (m, 7H), 7.60 (d, J=6.8 Hz, 2H).

### Example 313

### Synthesis of 3-(3-(4-cyanophenyl)-4-hydroxy-5,5-dimethyl-2-oxo-1-(p-tolyl)imidazolidin-4-yl)benzonitrile

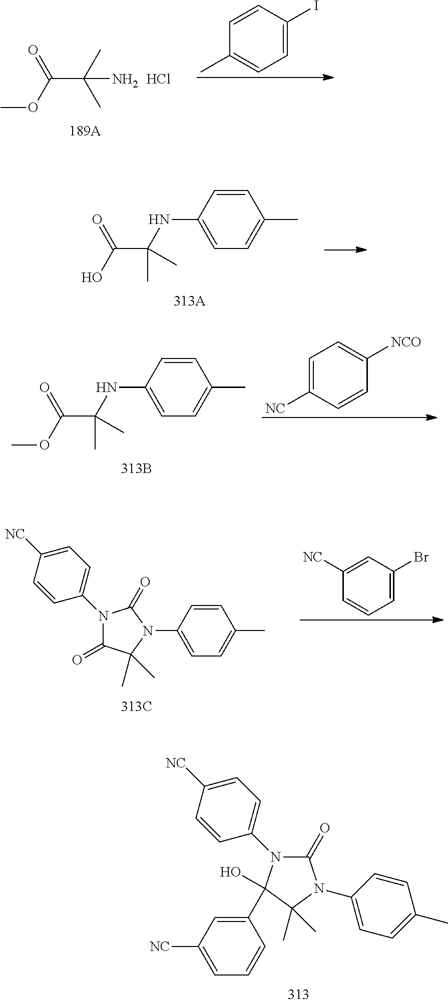

Compounds 313A, 313B, 313C, and 313 were synthesized by employing the procedures described for Compounds 224B, 268B, 224D, and 125 using Compounds 189A in the presence of 2-acetylcyclohexan-1-one, 1-iodo-4-methylbenzene, 313A, 313B, 4-isocyanatobenzonitrile, 313C, and 3-bromobenzonitrile in lieu of Compounds 224A without 2-acetylcyclohexan-1-one, 1-chloro-4-iodobenzene, 268A, 224C, 1-chloro-4-isocyanatobenzene, 2C, and 3-bromo-N,N-dimethylaniline. Compound 313A. LC-MS (ESI) m/z: 194 [M+H]⁺. Compound 313B. LC-MS (ESI) m/z: 208 [M+H]⁺. Compound 313C. LC-MS (ESI) m/z: 320 [M+H]⁺; ¹H-NMR (CDCl₃, 400 MHz): δ (ppm) 1.44 (s, 6H), 3.34 (s, 3H), 7.30-7.31 (m, 4H), 7.73-7.75 (m, 2H), 7.99-8.01 (m, 2H). Compound 313. LC-MS (ESI) m/z: 423 [M+H]⁺; ¹H-NMR (CDCl₃, 400 MHz): δ (ppm) 0.81 (s, 3H), 1.31 (s, 3H), 2.38 (s, 3H), 4.34 (s, 1H), 7.09-7.11 (m, 2H), 7.20-7.22 (m, 2H), 7.43-7.46 (m, 4H), 7.55-7.63 (m, 4H).

### Example 314

### Synthesis of 4-(5-hydroxy-4,4-dimethyl-2-oxo-3-(p-tolyl)-5-(3-(trifluoromethoxy)phenyl)imidazolidin-1-yl)benzonitrile

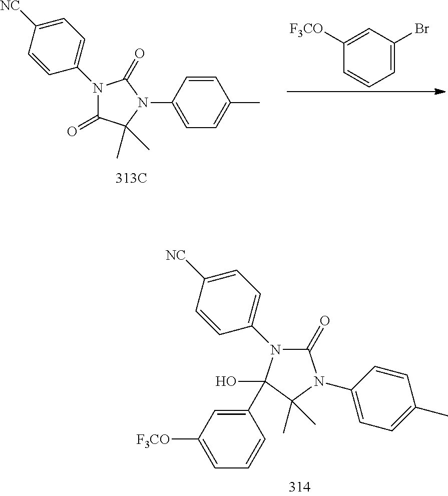 [SMILES: Cc1ccc(N2C(=O)N(c3ccc(C#N)cc3)C(=O)C2(C)C)cc1 ; FC(F)(F)Oc1cccc(Br)c1 ; Cc1ccc(N2C(=O)N(c3ccc(C#N)cc3)C(O)(c3cccc(OC(F)(F)F)c3)C2(C)C)cc1]

Compound 314 was synthesized by employing the procedure described for Compound 125 using Compound 313C and 1-bromo-3-(trifluoromethoxy)benzene in lieu of Compound 2C and 3-bromo-N,N-dimethylaniline. Compound 314. LC-MS (ESI) m/z: 482 [M+H]⁺; ¹H-NMR (CDCl₃, 400 MHz): δ (ppm) 0.80 (s, 3H), 1.29 (s, 3H), 2.37 (s, 3H), 4.55 (s, 1H), 7.07-7.09 (m, 2H), 7.15-7.22 (m, 4H), 7.30-7.35 (m, 2H), 7.42-7.45 (m, 2H), 7.57-7.59 (m, 2H).

### Example 315

### Synthesis of 7-(4-chlorophenyl)-8-hydroxy-5-(p-tolyl)-8-(3-(trifluoromethoxy)phenyl)-5,7-diazaspiro[3.4]octan-6-one

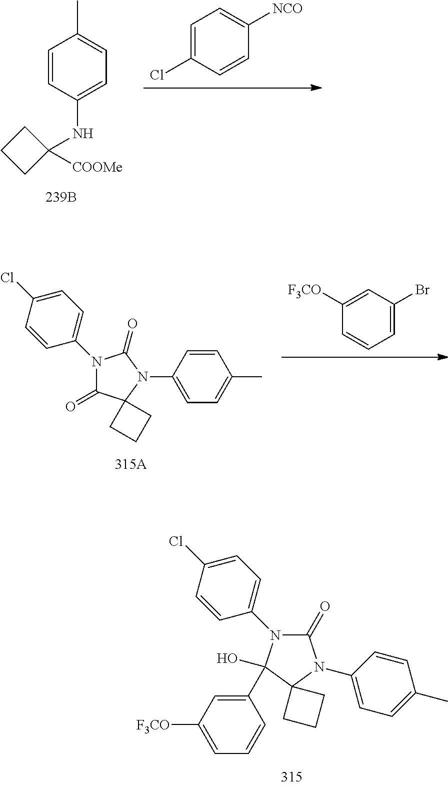

Compounds 315A and 315 were synthesized by employing the procedures described for Compounds 224D and 125 using Compounds 239B, 315A, and 1-bromo-3-(trifluoromethoxy)benzene in lieu of Compounds 224C, 2C, and 3-bromo-N,N-dimethylaniline. Compound 315A. LC-MS (ESI) m/z: 341 [M+H]⁺. Compound 315. LC-MS (ESI) m/z: 503 [M+H]⁺; ¹H-NMR (CDCl₃, 400 MHz): δ (ppm) 0.83-0.86 (m, 1H), 1.08-1.11 (m, 1H), 1.74-1.76 (m, 1H), 2.06-2.09 (m, 1H), 2.27-2.30 (m, 1H), 2.41 (s, 3H), 2.70-2.71 (m, 1H), 3.83 (s, 1H), 7.13-7.15 (m, 2H), 7.15-7.21 (m, 3H), 7.26-7.28 (m, 2H), 7.34-7.50 (m, 5H).

### Example 316

### Synthesis of 3-(7-(4-chlorophenyl)-8-hydroxy-6-oxo-5-(p-tolyl)-5,7-diazaspiro[3.4]octan-8-yl)benzonitrile

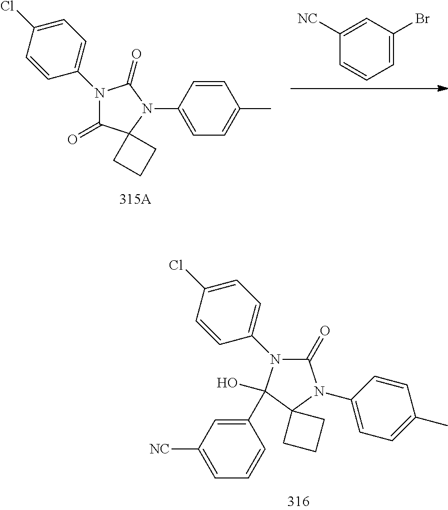 [SMILES: Cc1ccc(N2C(=O)N(c3ccc(Cl)cc3)C(=O)C23CCC3)cc1 ; N#Cc1cccc(Br)c1 ; Cc1ccc(N2C(=O)N(c3ccc(Cl)cc3)C(O)(c3cccc(C#N)c3)C23CCC3)cc1]

Compound 316 was synthesized by employing the procedure described for Compound 125 using Compound 315A and 3-bromobenzonitrile in lieu of Compound 2C and 3-bromo-N,N-dimethylaniline. Compound 316. LC-MS (ESI) m/z: 444 [M+H]⁺; ¹H-NMR (CDCl₃, 400 MHz): δ (ppm) 0.75-0.79 (m, 1H), 1.04-1.08 (m, 1H), 1.62-1.64 (m, 1H), 2.04-2.06 (m, 1H), 2.22-2.25 (m, 1H), 2.40 (s, 3H), 2.65-2.68 (m, 1H), 4.51 (s, 1H), 7.11-7.16 (m, 4H), 7.21-7.23 (m, 2H), 7.32-7.35 (m, 2H), 7.40-7.43 (m, 1H), 7.54-8.05 (m, 3H).

### Example 317

### Synthesis of 3-(3-(4-chlorophenyl)-4-hydroxy-5,5-dimethyl-2-oxo-1-(p-tolyl)imidazolidin-4-yl)benzonitrile

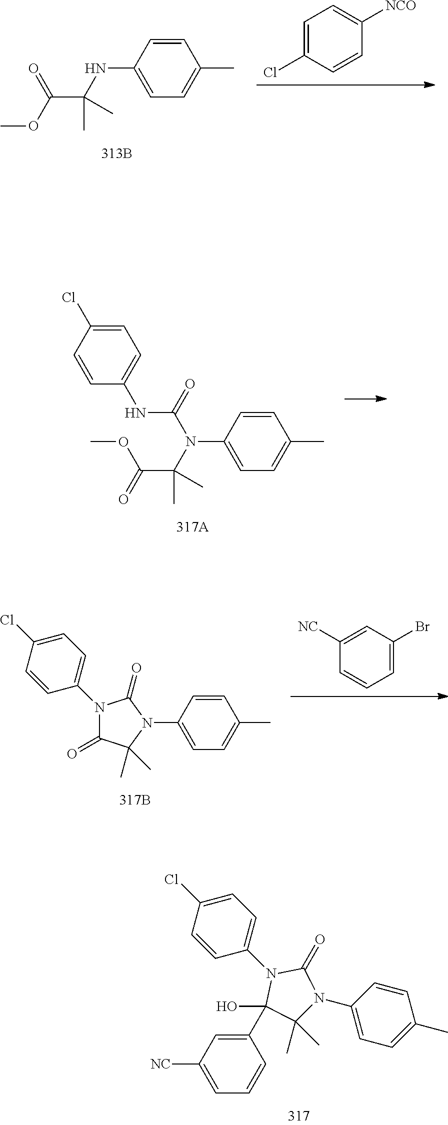

Compounds 317A, 317B, and 317 were synthesized by employing the procedures described for Compounds 224D, 224E, and 125 using Compounds 313B, 317A, 317B, and 3-bromobenzonitrile in lieu of Compounds 224C, 224D, 2C, and 3-bromo-N,N-dimethylaniline. Compound 317A. LC-MS (ESI) m/z: 361 [M+H]⁺. Compound 317B. LC-MS (ESI) m/z: 329 [M+H]⁺. Compound 317. LC-MS (ESI) m/z: 432 [M+H]⁺; ¹H-NMR (CDCl₃, 400 MHz): δ (ppm) 0.83 (s, 3H), 1.33 (s, 3H), 2.38 (s, 3H), 7.14-7.17 (m, 4H), 7.22 (d, J=6.4 Hz, 2H), 7.40-7.42 (m, 3H), 7.60 (d, J=6.0 Hz, 2H), 7.83 (s, 1H).

### Example 318

### Synthesis of 1-(4-chlorophenyl)-5-hydroxy-4,4-dimethyl-3-(p-tolyl)-5-(3-(trifluoromethoxy)phenyl)imidazolidin-2-one

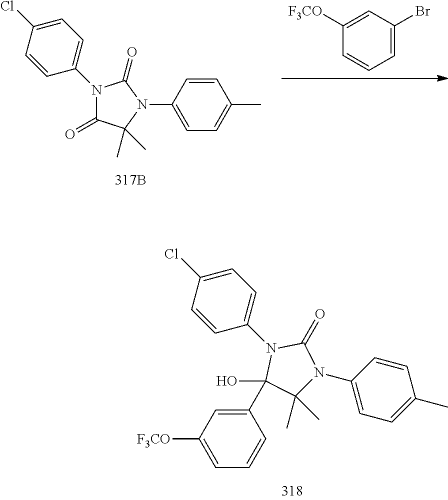 [SMILES: Cc1ccc(N2C(=O)N(c3ccc(Cl)cc3)C(=O)C2(C)C)cc1 ; FC(F)(F)Oc1cccc(Br)c1 ; Cc1ccc(N2C(=O)N(c3ccc(Cl)cc3)C(O)(c3cccc(OC(F)(F)F)c3)C2(C)C)cc1]

Compound 318 was synthesized by employing the procedure described for Compound 125 using Compound 317A and 1-bromo-3-(trifluoromethoxy)benzene in lieu of Compound 2C and 3-bromo-N,N-dimethylaniline. Compound 318. LC-MS (ESI) m/z: 491 [M+H]⁺. ¹H-NMR (CDCl₃, 400 MHz): δ (ppm) 0.83 (s, 3H), 1.32 (s, 3H), 2.38 (s, 3H), 3.82 (s, 1H), 7.12-7.18 (m, 5H), 7.21 (d, J=6.0 Hz, 2H), 7.32-7.33 (m, 3H), 7.41 (d, J=7.2 Hz, 2H).

### Example 319

### Synthesis of 4,4′-((4R)-4-ethyl-5-hydroxy-4-methyl-2-oxo-5-(3-(trifluoromethoxy)phenyl)imidazolidine-1,3-diyl)dibenzonitrile and 4,4′-((4S)-4-ethyl-5-hydroxy-4-methyl-2-oxo-5-(3-(trifluoromethoxy)phenyl)imidazolidine-1,3-diyl)dibenzonitrile

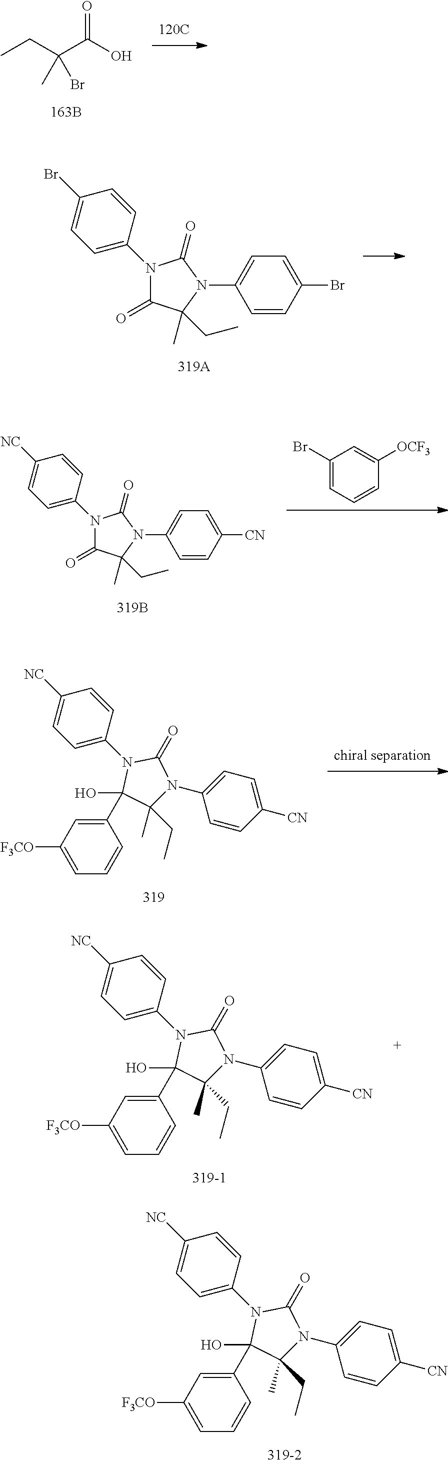

Compounds 319A, 319B, and 319 were synthesized by employing the procedures described for Compounds 120D, 180C, and 125 using Compounds 163B, 319A, 319B, and 1-bromo-3-(trifluoromethoxy)benzene in lieu of 2-bromo-2-methylpropanoic acid, Compounds 180B, 2C, and 3-bromo-N,N-dimethylaniline. Compound 319A. LC-MS (ESI) m/z: 451 [M+H]⁺; ¹H-NMR: (CDCl₃, 400 MHz): δ (ppm) 1.00 (t, J=7.2 Hz, 3H), 1.54 (s, 3H), 1.69-1.75 (m, 1H), 1.99-2.04 (m, 1H), 7.20-7.23 (m, 2H), 7.35-7.39 (m, 2H), 7.57-7.62 (m, 4H). Compound 319B. LC-MS (ESI) m/z: 345 [M+H]⁺.

Compound 319 was separated with chiral preparative HPLC to give Compounds 319-1 and 319-2. Compound 319-1. LC-MS (ESI) m/z: 507 [M+H]⁺; ¹H-NMR (CDCl₃, 400 MHz): δ (ppm) 0.89 (t, J=7.2 Hz, 3H), 0.97 (s, 3H), 1.82-1.87 (m, 1H), 1.99-2.07 (m, 1H), 4.24 (s, 1H), 7.20-7.22 (m, 1H), 7.37-7.54 (m, 9H), 7.67-7.71 (m, 2H). Chiral separation condition: MeOH (0.2% Methanol Ammonia), OD-H (4.6*100 mm, 5 μm), retention time: 1.31 min. Compound 319-2. LC-MS (ESI) m/z: 507 [M+H]⁺; ¹H-NMR (CDCl₃, 400 MHz): δ (ppm) 0.89 (t, J=7.2 Hz, 3H), 0.97 (s, 3H), 1.84-1.88 (m, 1H), 1.99-2.06 (m, 1H), 4.20 (s, 1H), 7.20-7.22 (m, 1H), 7.36-7.47 (m, 9H), 7.68-7.72 (m, 2H). Chiral separation condition: MeOH (0.2% Methanol Ammonia), OD-H (4.6*100 mm, 5 μm), retention time: 2.22 min.

### Example 320

### Synthesis of 4,4′-((5R)-4-hydroxy-5-methyl-2-oxo-5-propyl-4-(3-(trifluoromethoxy)phenyl)imidazolidine-1,3-diyl)dibenzonitrile and 4,4′-((5S)-4-hydroxy-5-methyl-2-oxo-5-propyl-4-(3-(trifluoromethoxy)phenyl)imidazolidine-1,3-diyl)dibenzonitrile

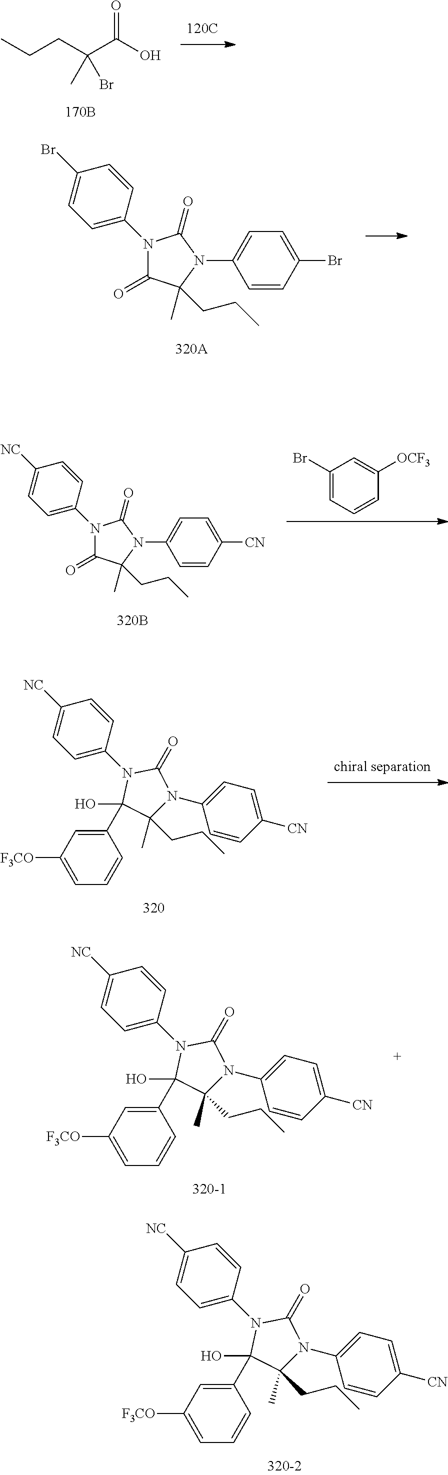

Compounds 320A, 320B, and 320 were synthesized by employing the procedures described for Compounds 120D, 180C, and 125 using Compounds 170B, 320A, 320B, and 1-bromo-3-(trifluoromethoxy)benzene in lieu of 2-bromo-2-methylpropanoic acid, Compounds 180B, 2C, and 3-bromo-N,N-dimethylaniline. Compound 320A. LC-MS (ESI) m/z: 465 [M+H]⁺; ¹H-NMR: (CDCl₃, 400 MHz): δ (ppm) 0.94 (t, J=7.2 Hz, 3H), 1.30-1.33 (m, 1H), 1.48-1.51 (m, 1H), 1.53 (s, 3H), 1.62-1.64 (m, 1H), 1.90-1.95 (m, 1H), 7.18-7.21 (m, 2H), 7.35-7.38 (m, 2H), 7.58-7.61 (m, 4H). Compound 320B. LC-MS (ESI) m/z: 359 [M+H]⁺.

Compound 320 was separated with chiral preparative HPLC to give Compounds 320-1 and 320-2. Compound 320-1. LC-MS (ESI) m/z: 521 [M+H]⁺; ¹H-NMR (CDCl₃, 400 MHz): δ (ppm) 0.87 (t, J=7.2 Hz, 3H), 0.97 (s, 3H), 1.04-1.12 (m, 1H), 1.39-1.48 (m, 1H), 1.72-1.79 (m, 1H), 1.89-2.00 (m, 1H), 3.97 (s, 1H), 7.21-7.24 (m, 1H), 7.33-7.48 (m, 9H), 7.70-7.74 (m, 2H). Chiral separation condition: MeOH (0.2% Methanol Ammonia), OD-H (4.6*100 mm, 5 μm), retention time: 1.02 min. (53%), 1.91 min. (40%). Compound 320-2. LC-MS (ESI) m/z: 521 [M+H]⁺; ¹H-NMR (CDCl₃, 400 MHz): δ (ppm) 0.88 (t, J=7.2 Hz, 3H), 0.97 (s, 3H), 1.06-1.10 (m, 1H), 1.40-1.46 (m, 1H), 1.71-1.78 (m, 1H), 1.89-1.94 (m, 1H), 4.06 (s, 1H), 7.20-7.22 (m, 1H), 7.33-7.54 (m, 9H), 7.69-7.73 (m, 2H). Chiral separation condition: MeOH (0.2% Methanol Ammonia), OD-H (4.6*100 mm, 5 μm), retention time: 1.65 min (88%).

### Example 321

### Synthesis of 3-(1,3-bis(4-chlorophenyl)-5,5-diethyl-4-hydroxy-2-oxoimidazolidin-4-yl)benzonitrile

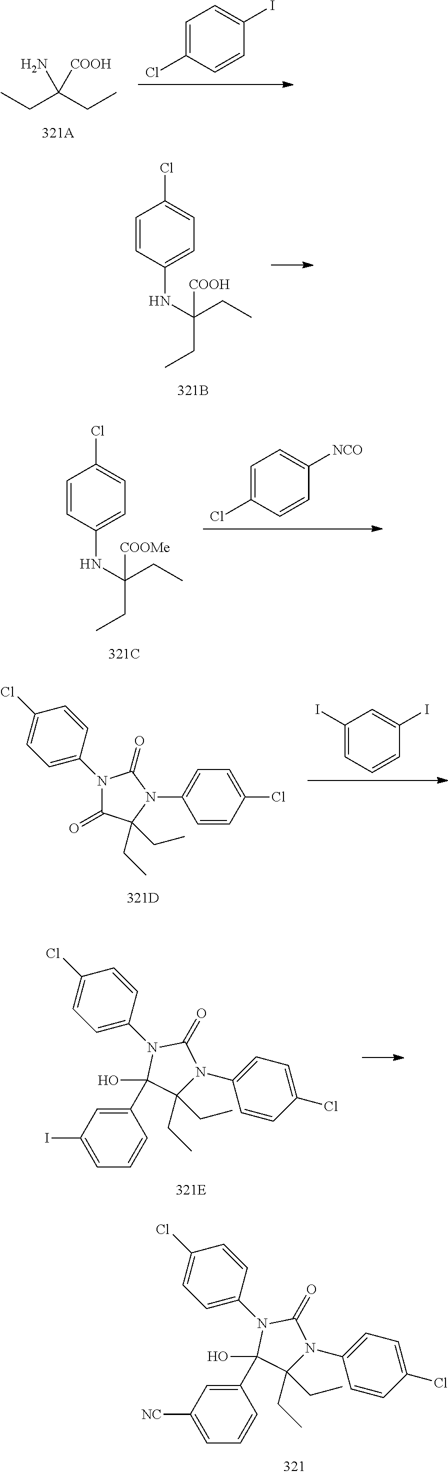

Compounds 321B, 321C, 321D, 321E, and 321 were synthesized by employing the procedures described for Compounds 224B, 224C, 224D, 125, and 180C using Compounds 321A in the presence of 2-acetylcyclohexan-1-one, 321B, 321C, 321D, 1,3-diiodobenzene, and 321E in lieu of Compounds 224A without 2-acetylcyclohexan-1-one, 224B, 224C, 2C, 3-bromo-N,N-dimethylaniline, and 180B. Compound 321B. LC-MS (ESI) m/z: 240 [M−H]⁻. Compound 321C. LC-MS (ESI) m/z: 256 [M+H]⁺. Compound 321D. LC-MS (ESI) m/z: 377 [M+H]⁺. Compound 321E. LC-MS (ESI) m/z: 581 [M+H]⁺. Compound 321. LC-MS (ESI) m/z: 480 [M+H]⁺; ¹H-NMR (CDCl₃, 400 MHz): δ (ppm) 0.55 (t, J=7.2 Hz, 3H), 1.02 (t, J=7.2 Hz, 3H), 1.41-1.44 (m, 2H), 1.84-1.94 (m, 1H), 2.07-2.17 (m, 1H), 3.93 (s, 1H), 7.12-7.22 (m, 6H), 7.34-7.38 (m, 2H), 7.46 (t, J=8.0 Hz, 1H), 7.62-7.76 (m, 2H), 7.91 (s, 1H).

### Example 322

### Synthesis of 4-(3-(4-chlorophenyl)-4-hydroxy-5,5-dimethyl-2-oxo-4-(3-(trifluoromethoxy)phenyl)imidazolidin-1-yl)benzonitrile

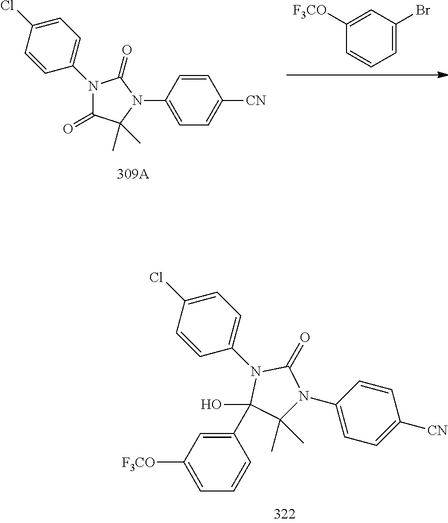 [SMILES: CC1(C)C(=O)N(c2ccc(Cl)cc2)C(=O)N1c1ccc(C#N)cc1 ; FC(F)(F)Oc1cccc(Br)c1 ; CC1(C)N(c2ccc(C#N)cc2)C(=O)N(c2ccc(Cl)cc2)C1(O)c1cccc(OC(F)(F)F)c1]

Compound 322 was synthesized by employing the procedure described for Compound 125 using Compound 309A and 1-bromo-3-(trifluoromethoxy)benzene in lieu of Compound 2C and 3-bromo-N,N-dimethylaniline. Compound 322. LC-MS (ESI) m/z: 502 [M+H]⁺; ¹H-NMR (CDCl₃, 400 MHz): δ (ppm) 0.82 (s, 3H), 1.37 (s, 3H), 4.21 (s, 1H), 7.16-7.19 (m, 3H), 7.26-7.37 (m, 7H), 7.65 (d, J=6.8 Hz, 2H).

### Example 323

### Synthesis of 3-(1-(4-chlorophenyl)-3-(4-cyanophenyl)-4-hydroxy-5,5-dimethyl-2-oxoimidazolidin-4-yl)benzonitrile

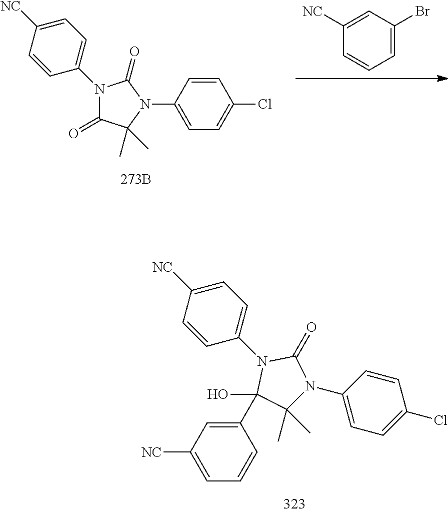 [SMILES: CC1(C)C(=O)N(c2ccc(C#N)cc2)C(=O)N1c1ccc(Cl)cc1 ; N#Cc1cccc(Br)c1 ; CC1(C)N(c2ccc(Cl)cc2)C(=O)N(c2ccc(C#N)cc2)C1(O)c1cccc(C#N)c1]

Compound 323 was synthesized by employing the procedure described for Compound 125 using Compound 273B and 3-bromobenzonitrile in lieu of Compound 2C and 3-bromo-N,N-dimethylaniline. Compound 323. LC-MS (ESI) m/z: 443 [M+H]⁺; ¹H-NMR (CDCl₃, 400 MHz): δ (ppm) 0.79 (s, 3H), 1.31 (s, 3H), 4.678 (s, 1H), 7.14 (d, J=7.2 Hz, 2H), 7.34-7.46 (m, 6H), 7.53-7.63 (m, 4H).

### Example 324

### Synthesis of 1,3-bis(4-chlorophenyl)-4-hydroxy-5,5-dimethyl-4-(3-(oxetan-2-yl)phenyl)imidazolidin-2-one and 1,3-bis(4-chlorophenyl)-4-hydroxy-5,5-dimethyl-4-(3-(oxetan-3-yl)phenyl)imidazolidin-2-one

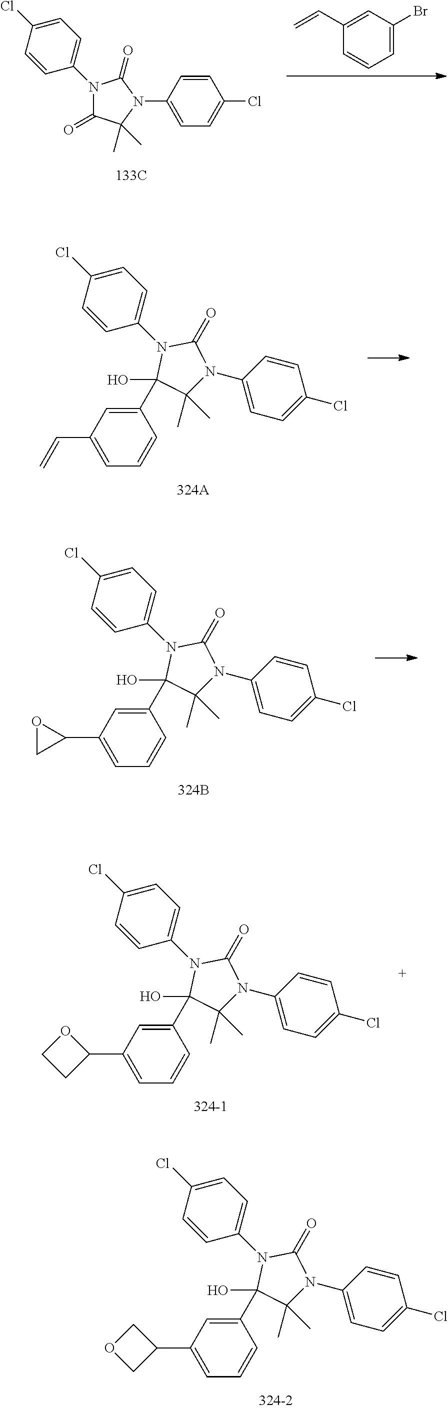

Compound 324A was synthesized by employing the procedure described for Compound 125 using Compound 133C and 1-bromo-3-vinylbenzene in lieu of Compound 2C and 3-bromo-N,N-dimethylaniline. Compound 324A. LC-MS (ESI) m/z: 453 [M+H]⁺; ¹H-NMR (400 MHz, CDCl₃): δ (ppm) 0.81 (s, 3H), 1.34 (m, 3H), 3.95 (s, 1H), 5.25 (d, J=10.8 Hz, 1H), 5.68 (d, J=17.2 Hz, 1H), 6.67 (dd, J₁=17.2 Hz, J₂=17.6 Hz, 1H), 7.15-7.19 (m, 4H), 7.22-7.23 (m, 2H), 7.34-7.36 (m, 3H), 7.40-7.42 (m, 2H), 7.49 (s, 1H).

To a solution of Compound 324A (550 mg, 1.21 mmol) in DCM (20 mL) was added mCPBA (838 mg, 4.85 mmol), stirred at room temperature for 4 hours, diluted with DCM (100 mL), washed with saturated NaHCO₃ solution (50 mL), dried over anhydrous Na₂SO₄, filtered, and concentrated under vacuum. The residue was purified with flash column chromatography on silica gel (ethyl acetate in petroleum ether, 20% v/v) to give Compound 324B. LC-MS (ESI) m/z: 469 [M+H]⁺; ¹H-NMR (400 MHz, CDCl₃): δ (ppm) 0.78-0.79 (m, 3H), 1.32-1.34 (m, 3H), 2.46-2.80 (m, 1H), 3.08-3.15 (m, 1H), 3.75-3.83 (m, 1H), 4.05-4.13 (m, 1H), 7.14-7.24 (m, 7H), 7.33-7.41 (m, 5H).

To a suspension of trimethylsulfoxonium iodide (132 mg, 0.6 mmol) in t-BuOH (4 mL) was added t-BuOK (67 mg, 0.6 mmol). After the mixture was stirred at 50° C. for 1 hour, to it was added Compound 324B (140 mg, 0.3 mmol) and stirred at 50° C. overnight. The reaction mixture was diluted with DCM (100 mL), washed with water (100 mL) and brine (50 mL), dried over anhydrous Na₂SO₄, filtered, and concentrated under vacuum. The residue was purified with flash column chromatography on silica gel (ethyl acetate in petroleum ether, 20% v/v) and preparative HPLC to give Compounds 324-1 and 324-2. Compound 324-1. LC-MS (ESI) m/z: 483 [M+H]⁺; ¹H-NMR (400 MHz, CDCl₃): δ (ppm) 0.82-0.83 (m, 3H), 1.36-1.37 (m, 3H), 2.30-2.57 (m, 1H), 2.94-3.02 (m, 1H), 3.58-3.62 (m, 1H), 4.48-4.64 (m, 1H), 4.75-4.84 (m, 1H), 5.71-5.78 (m, 1H), 7.16-7.18 (m, 2H), 7.21-7.23 (m, 2H), 7.29-7.34 (m, 2H), 7.38-7.45 (m, 5H), 7.48-7.53 (m, 1H). Compound 324-2. LC-MS (ESI) m/z: 483 [M+H]⁺; ¹H-NMR (400 MHz, CDCl₃): δ (ppm) 0.82 (s, 3H), 1.35 (s, 3H), 1.56-1.58 (m, 1H), 3.83 (s, 1H), 4.42-4.43 (m, 2H), 5.33-5.37 (m, 2H), 7.16-7.21 (m, 4H), 7.28-7.32 (m, 2H), 7.36-7.43 (m, 5H) 7.49 (s, 1H).

### Example 325

### Synthesis of 4-(3-(4-bromophenyl)-4-hydroxy-2-oxo-4-(3-(trifluoromethoxy)phenyl)-1,3-diazaspiro[4.4]nonan-1-yl)benzonitrile

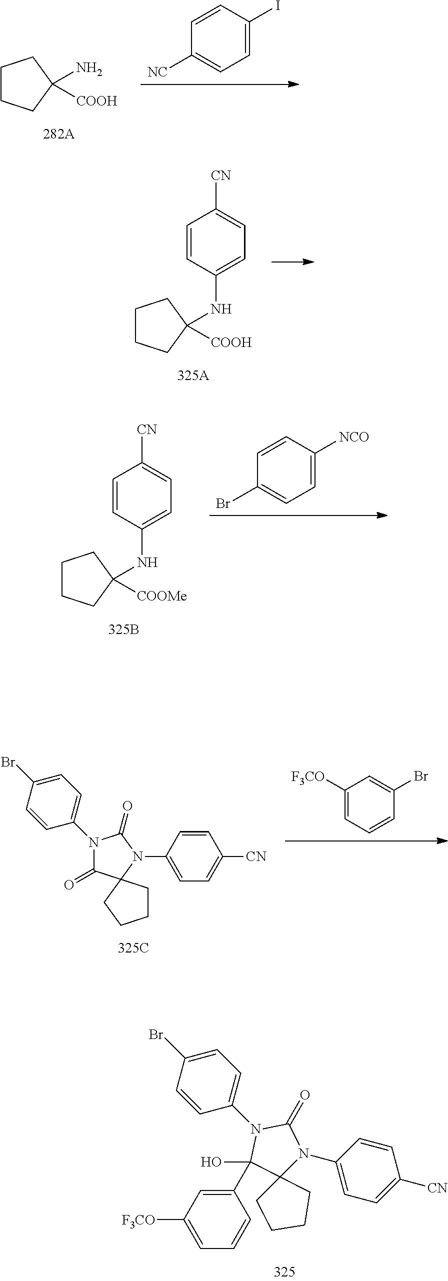

Compounds 325A, 325B, 325C, and 325 were synthesized by employing the procedures described for Compounds 224B, 224C, 224D, and 125 using 4-iodobenzonitrile, Compounds 282A in the presence of 2-acetylcyclohexan-1-one, 325A, 325B, 1-bromo-4-isocyanatobenzene, 325C, and 1-bromo-3-(trifluoromethoxy)benzene in lieu of 1-chloro-4-iodobenzene, Compounds 224A without 2-acetylcyclohexan-1-one, 224B, 224C, 1-chloro-4-isocyanatobenzene, 2C, and 3-bromo-N,N-dimethylaniline. Compound 325A. LC-MS (ESI) m/z: 229 [M−H]⁻. Compound 325B. LC-MS (ESI) m/z: 245 [M+H]⁺. Compound 325C. LC-MS (ESI) m/z: 410 [M+H]⁺. Compound 325. LC-MS (ESI) m/z: 572 [M+H]⁺; ¹H-NMR (CDCl₃, 400 MHz): δ (ppm) 0.44-0.56 (m, 1H), 1.14-1.32 (m, 2H), 1.46-1.56 (m, 1H), 1.60-1.80 (m, 2H), 1.98-2.07 (m, 1H), 2.40-2.50 (m, 1H), 3.66 (s, 1H), 7.18-7.22 (m, 2H), 7.28-7.38 (m, 6H), 7.46-7.51 (m, 2H), 7.70-7.75 (m, 2H).

### Example 326

### Synthesis of 4,4′-(4-hydroxy-2-oxo-4-(3-(trifluoromethoxy)phenyl)-1,3-diazaspiro[4.4]nonane-1,3-diyl)dibenzonitrile

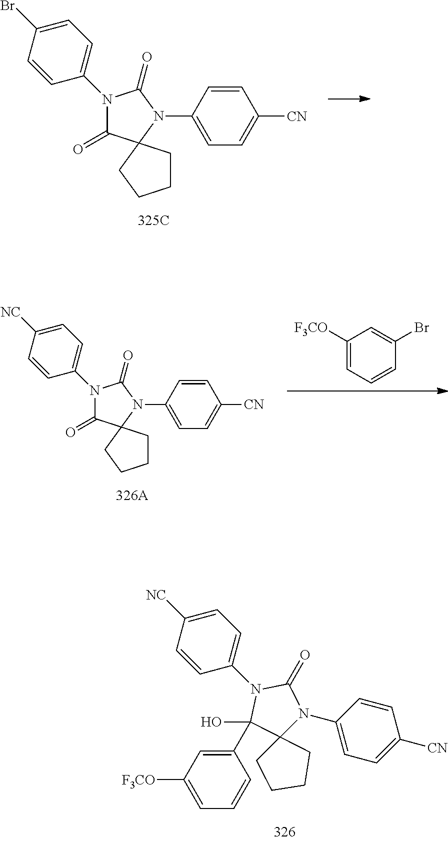 [SMILES: N#Cc1ccc(N2C(=O)N(c3ccc(Br)cc3)C(=O)C23CCCC3)cc1 ; N#Cc1ccc(N2C(=O)N(c3ccc(C#N)cc3)C3(CCCC3)C2=O)cc1 ; FC(F)(F)Oc1cccc(Br)c1 ; N#Cc1ccc(N2C(=O)N(c3ccc(C#N)cc3)C(O)(c3cccc(OC(F)(F)F)c3)C23CCCC3)cc1]

Compounds 326A and 326 were synthesized by employing the procedures described for Compounds 180C and 125 using Compounds 325C, 326A, and 1-bromo-3-(trifluoromethoxy)benzene in lieu of Compounds 180B, 2C, and 3-bromo-N,N-dimethylaniline. Compound 326A. LC-MS (ESI) m/z: 357 [M+H]⁺. Compound 326. LC-MS (ESI) m/z: 519 [M+H]⁺; ¹H-NMR (CDCl₃, 400 MHz): δ (ppm) 0.44-0.56 (m, 1H), 1.14-1.30 (m, 2H), 1.46-1.56 (m, 1H), 1.62-1.79 (m, 2H), 1.98-2.08 (m, 1H), 2.40-2.50 (m, 1H), 4.06 (s, 1H), 7.20-7.24 (m, 1H), 7.30-7.57 (m, 9H), 7.70-7.75 (m, 2H).

### Example 327

### Synthesis of 4-hydroxy-1,3-di-p-tolyl-4-(3-(trifluoromethoxy)phenyl)-1,3-diazaspiro[4.4]nonan-2-one

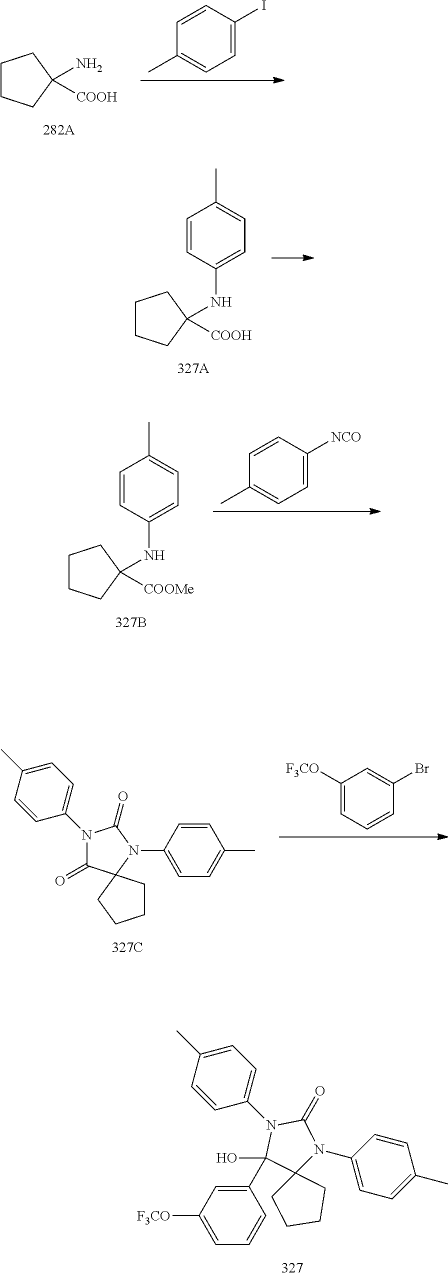

Compounds 327A, 327B, 327C, and 327 were synthesized by employing the procedures described for Compounds 224B, 224C, 224D, and 125 using 1-iodo-4-methylbenzene, Compounds 282A in the presence of 2-acetylcyclohexan-1-one, 327A, 327B, 1-isocyanato-4-methylbenzene, 327C, and 1-bromo-3-(trifluoromethoxy)benzene in lieu of 1-chloro-4-iodobenzene, Compounds 224A without 2-acetylcyclohexan-1-one, 224B, 224C, 1-chloro-4-isocyanatobenzene, 2C, and 3-bromo-N,N-dimethylaniline. Compound 327A. LC-MS (ESI) m/z: 220 [M+H]⁺. Compound 327B. LC-MS (ESI) m/z: 234 [M+H]⁺; ¹H-NMR (CDCl₃, 400 MHz): δ (ppm) 1.77-1.81 (m, 4H), 1.91-1.96 (m, 2H), 2.22 (s, 3H), 2.27-2.32 (m, 2H), 3.66 (s, 3H), 6.45 (d, J=6.8 Hz, 2H), 6.95 (d, J=6.4 Hz, 2H). Compound 327C. LC-MS (ESI) m/z: 335 [M+H]⁺. Compound 327. LC-MS (ESI) m/z: 497 [M+H]⁺; ¹H-NMR (CDCl₃, 400 MHz): δ (ppm) 0.50-0.58 (m, 1H), 1.13-1.90 (m, 2H), 1.36-1.42 (m, 1H), 1.64-1.71 (m, 2H), 1.98-2.03 (m, 1H), 2.24 (s, 3H), 2.38 (s, 4H), 3.39 (s, 1H), 7.01 (d, J=6.4 Hz, 2H), 7.16-7.17 (m, 1H), 7.21-7.22 (m, 4H), 7.28-7.33 (m, 3H), 7.44-7.46 (m, 2H).

### Example 328

### Synthesis of 3-(4-hydroxy-1,3-bis(4-methoxyphenyl)-5,5-dimethyl-2-oxoimidazolidin-4-yl)benzonitrile

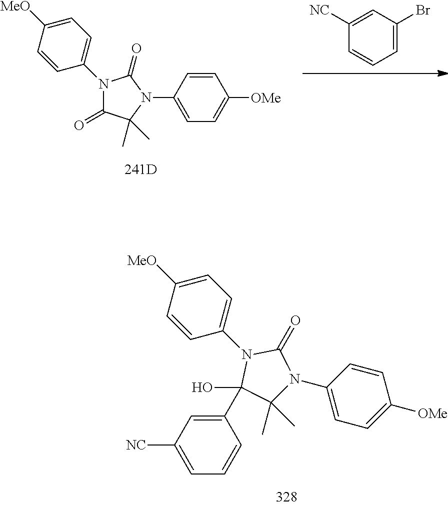 [SMILES: COc1ccc(N2C(=O)N(c3ccc(OC)cc3)C(C)(C)C2=O)cc1 ; N#Cc1cccc(Br)c1 ; COc1ccc(N2C(=O)N(c3ccc(OC)cc3)C(O)(c3cccc(C#N)c3)C2(C)C)cc1]

Compound 328 was synthesized by employing the procedure described for Compound 125 using Compound 241D and 3-bromobenzonitrile in lieu of Compound 2C and 3-bromo-N,N-dimethylaniline. Compound 328. LC-MS (ESI) m/z: 444 [M+H]⁺; ¹H-NMR (CD₃OD, 400 MHz): δ (ppm) 0.86 (s, 3H), 1.34 (s, 3H), 3.74 (s, 3H), 3.84 (s, 3H), 6.83 (d, J=9.2 Hz, 2H), 7.02 (d, J=8.8 Hz, 2H), 7.31 (d, J=8.8 Hz, 2H), 7.46-7.50 (m, 3H), 7.65 (d, J=7.6 Hz, 1H), 7.84 (d, J=8.0 Hz, 1H), 7.91 (s, 1H).

### Example 329

### Synthesis of (5S)-4-hydroxy-5-isopropyl-1,3-bis(4-methoxyphenyl)-4-(3-(trifluoromethoxy)phenyl)imidazolidin-2-one and (5R)-4-hydroxy-5-isopropyl-1,3-bis(4-methoxyphenyl)-4-(3-(trifluoromethoxy)phenyl)imidazolidin-2-one

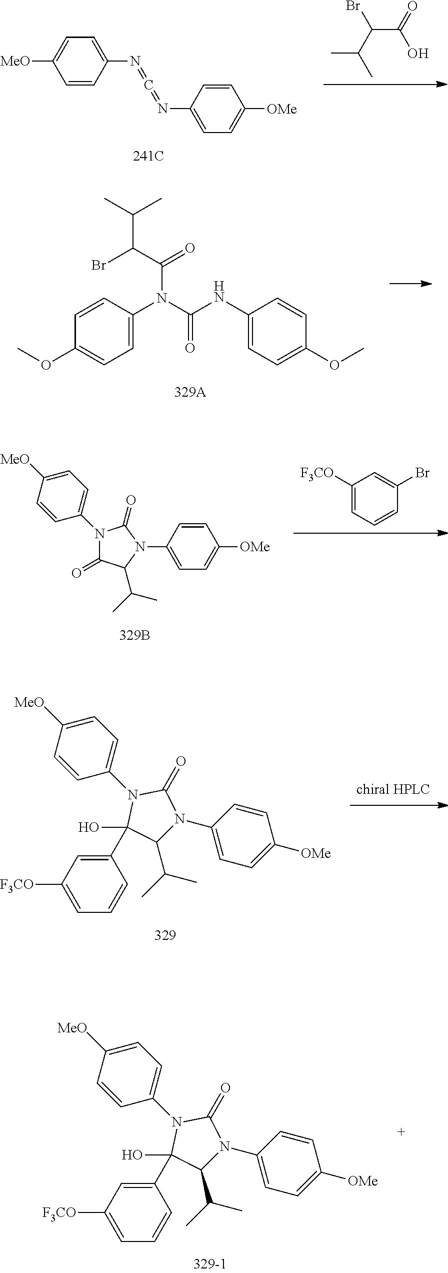

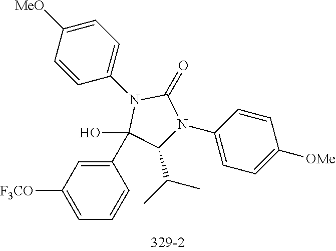 [SMILES: COc1ccc(N2C(=O)N(c3ccc(OC)cc3)C(O)(c3cccc(OC(F)(F)F)c3)[C@H]2C(C)C)cc1]

To a stirred solution of 2-bromo-2-methylpropanoic acid (0.21 g, 1.2 mmol) in 1,4-dioxane (10 mL) was added Compound 241C (0.36 g, 1.4 mmol), followed by 2,4,6-trimethylpyridine (0.15 g, 1.2 mmol) at room temperature. The mixture was stirred at room temperature overnight, quenched with water (30 mL), and extracted with dichloromethane (50 mL×3). The combined organic layers was dried over anhydrous sodium sulfate, filtered, and concentrated to give a crude Compound 329A. LC-MS (ESI) m/z: 435 [M+H]⁺.

A mixture of Compound 329A (0.4 g, 0.9 mmol) and K₂CO₃ (0.25 g, 1.8 mmol) in acetonitrile (10 mL) was stirred at room temperature for 2 hours. The mixture was quenched with water (30 mL) and extracted with dichloromethane (50 mL×3). The combined organic layers was dried over anhydrous sodium sulfate, filtered, and concentrated under vacuum. The residue was purified with preparative HPLC to furnish Compound 329B. LC-MS (ESI) m/z: 355 [M+H]⁺; H-NMR (CD₃OD, 400 MHz): δ (ppm) 0.91 (d, J=6.8 Hz, 3H), 1.20 (d, J=7.2 Hz, 3H), 2.18-2.22 (m, 1H), 3.84 (d, J=7.2 Hz, 6H), 4.77 (d, J=3.2 Hz, 1H), 7.00-7.06 (m, 4H), 7.27-7.31 (m, 2H), 7.40-7.44 (m, 2H).

Compound 329 was synthesized by employing the procedure described for Compound 125 using Compound 329B and 1-bromo-3-(trifluoromethoxy)benzene in lieu of Compound 2C and 3-bromo-N,N-dimethylaniline. Compound 329. LC-MS (ESI) m/z: 517 [M+H]⁺; ¹H-NMR (CD₃OD, 400 MHz): δ (ppm) 0.85 (d, J=6.8 Hz, 3H), 0.90 (d, J=7.2 Hz, 3H), 2.23-2.28 (m, 1H), 3.70 (s, 3H), 3.82 (s, 3H), 4.32 (d, J=5.2 Hz, 1H), 6.74-6.77 (m, 2H), 6.98-7.01 (m, 2H), 7.15-7.19 (m, 3H), 7.33-7.37 (m, 2H), 7.45 (t, J=8.0 Hz, 1H), 7.58 (s, 1H), 7.67 (d, J=8.0 Hz, 1H).

Compound 329 was separated with chiral preparative HPLC to give Compounds 329-1 and Compound 329-2. Compound 329-1. LC-MS (ESI) m/z: 517 [M+H]⁺; ¹H-NMR (CD₃OD, 400 MHz): δ (ppm) 0.88 (d, J=7.2 Hz, 3H), 0.92 (d, J=7.2 Hz, 3H), 2.25-2.30 (m, 1H), 3.69 (s, 3H), 3.81 (s, 3H), 4.34 (d, J=5.2 Hz, 1H), 6.76 (dd, J=3.2, 12.4 Hz, 2H), 7.00 (dd, J=3.2, 12.0 Hz, 2H), 7.18-7.22 (m, 3H), 7.35-7.42 (m, 3H), 7.61 (s, 1H), 7.68 (d, J=8.0 Hz, 1H). Chiral separation condition: MeOH contained 0.2% Methanol Ammonia, Cellulose-SC (4.6*100 mm, 5 μm), retention time: 1.59 min. Compound 329-2. LC-MS (ESI) m/z: 517 [M+H]⁺; ¹H-NMR (CD₃OD, 400 MHz): δ (ppm) 0.88 (d, J=6.8 Hz, 3H), 0.92 (d, J=6.8 Hz, 3H), 2.26-2.29 (m, 1H), 3.69 (s, 3H), 3.81 (s, 3H), 4.34 (d, J=5.2 Hz, 1H), 6.74-6.78 (m, 2H), 7.00 (dd, J=3.2, 12.0 Hz, 2H), 7.18-7.22 (m, 3H), 7.35-7.46 (m, 3H), 7.61 (s, 1H), 7.68 (d, J=7.6 Hz, 1H). Chiral separation condition: MeOH contained 0.2% Methanol Ammonia, Cellulose-SC (4.6*100 mm, 5 μm), retention time: 2.60 min.

### Example 330

### Synthesis of 3-(4-hydroxy-5-isopropyl-1,3-bis(4-methoxyphenyl)-2-oxoimidazolidin-4-yl)benzonitrile

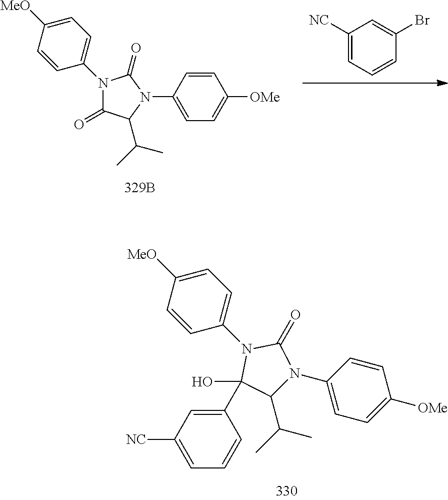 [SMILES: COc1ccc(N2C(=O)C(C(C)C)N(c3ccc(OC)cc3)C2=O)cc1 ; N#Cc1cccc(Br)c1 ; COc1ccc(N2C(=O)N(c3ccc(OC)cc3)C(O)(c3cccc(C#N)c3)C2C(C)C)cc1]

Compound 330 was synthesized by employing the procedure described for Compound 125 using Compound 329B and 3-bromobenzonitrile in lieu of Compound 2C and 3-bromo-N,N-dimethylaniline. Compound 330. LC-MS (ESI) m/z: 458 [M+H]⁺; ¹H-NMR (CD₃OD, 400 MHz): δ (ppm) 0.84 (d, J=7.2 Hz, 3H), 0.90 (d, J=6.8 Hz, 3H), 2.23-2.26 (m, 1H), 3.71 (s, 3H), 3.83 (s, 3H), 4.36 (d, J=5.2 Hz, 1H), 6.75-6.78 (m, 2H), 7.00 (d, J=8.8 Hz, 2H), 7.18-7.21 (m, 2H), 7.38-7.40 (m, 2H), 7.52-7.56 (m, 1H), 7.63-7.65 (m, 1H), 8.01-8.06 (m, 2H).

### Example 331

### Synthesis of 5-((5S)-1,3-bis(4-chlorophenyl)-4-hydroxy-5-methyl-2-oxo-5-(trifluoromethyl)imidazolidin-4-yl)isophthalonitrile and 5-((5R)-1,3-bis(4-chlorophenyl)-4-hydroxy-5-methyl-2-oxo-5-(trifluoromethyl)imidazolidin-4-yl)isophthalonitrile

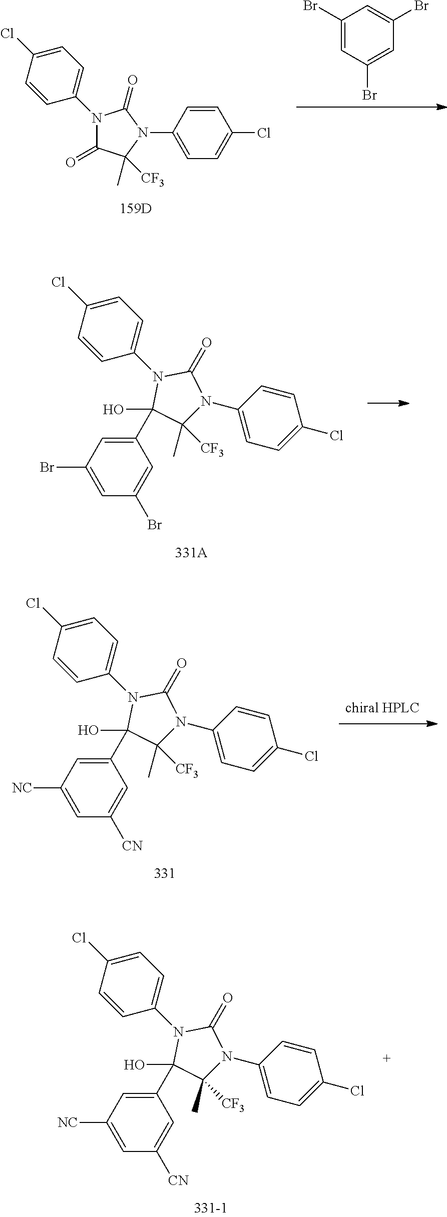 [SMILES: CC1(C(F)(F)F)C(=O)N(c2ccc(Cl)cc2)C(=O)N1c1ccc(Cl)cc1 ; Brc1cc(Br)cc(Br)c1 ; CC1(C(F)(F)F)N(c2ccc(Cl)cc2)C(=O)N(c2ccc(Cl)cc2)C1(O)c1cc(Br)cc(Br)c1 ; CC1(C(F)(F)F)N(c2ccc(Cl)cc2)C(=O)N(c2ccc(Cl)cc2)C1(O)c1cc(C#N)cc(C#N)c1 ; C[C@]1(C(F)(F)F)N(c2ccc(Cl)cc2)C(=O)N(c2ccc(Cl)cc2)C1(O)c1cc(C#N)cc(C#N)c1]

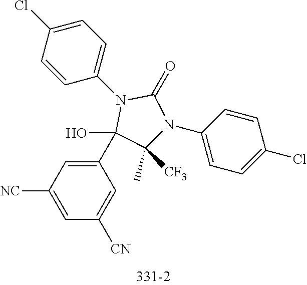 [SMILES: C[C@@]1(C(F)(F)F)N(c2ccc(Cl)cc2)C(=O)N(c2ccc(Cl)cc2)C1(O)c1cc(C#N)cc(C#N)c1]

Compounds 331A and 331 were synthesized by employing the procedures described for Compounds 125 and 180C using 1,3,5-tribromobenzene, Compounds 159D, and 331A in lieu of 3-bromo-N,N-dimethylaniline, Compounds 2C, and 180B. Compound 331A. LC-MS (ESI) m/z: 637 [M+H]⁺; ¹H-NMR (CDCl₃, 400 MHz): δ (ppm) 1.07 (s, 3H), 3.62 (s, 1H), 7.17-7.26 (m, 8H), 7.40 (d, J=8.4 Hz, 2H), 7.73 (s, 1H). Compound 331. LC-MS (ESI) m/z: 531 [M+H]⁺; ¹H-NMR (CDCl₃, 400 MHz): δ (ppm) 1.11 (s, 3H), 4.16 (s, 1H), 7.22-7.26 (m, 6H), 7.31 (d, J=8.4 Hz, 2H), 7.46 (d, J=8.4 Hz, 2H), 7.94 (s, 1H).

Compound 331 was separated with chiral preparative HPLC to afford Compounds 331-1 and 331-2. Compound 331-1. LC-MS (ESI) m/z: 531 [M+H]⁺; ¹H-NMR (CD₃OD, 400 MHz): δ (ppm) 1.70 (s, 3H), 7.32-7.34 (m, 2H), 7.44-7.46 (m, 2H), 7.51-7.55 (s, 4H), 7.81 (brs, 1H), 8.20 (s, 1H), 8.50 (brs, 1H). Chiral separation condition: MeOH contained 0.2% methanol ammonia, (R,R)-Whelk-Ol (4.6*100 mm, 5 μm), retention time: 2.94 min. Compound 331-2. LC-MS (ESI) m/z: 531 [M+H]⁺; ¹H-NMR (CD₃OD, 400 MHz): δ (ppm) 1.70 (s, 3H), 7.32-7.34 (m, 2H), 7.44-7.54 (m, 6H), 7.75 (brs, 1H), 8.20 (s, 1H), 8.50 (brs, 1H). Chiral separation condition: co-solvent: MeOH contained 0.2% methanol ammonia, (R,R)-Whelk-Ol (4.6*100 mm, 5 μm), retention time: 4.01 min.

### Example 332

### Synthesis of 5-(4-chlorophenyl)-8-hydroxy-7-phenyl-8-(3-(trifluoromethoxy)phenyl)-5,7-diazaspiro[3.4]octan-6-one

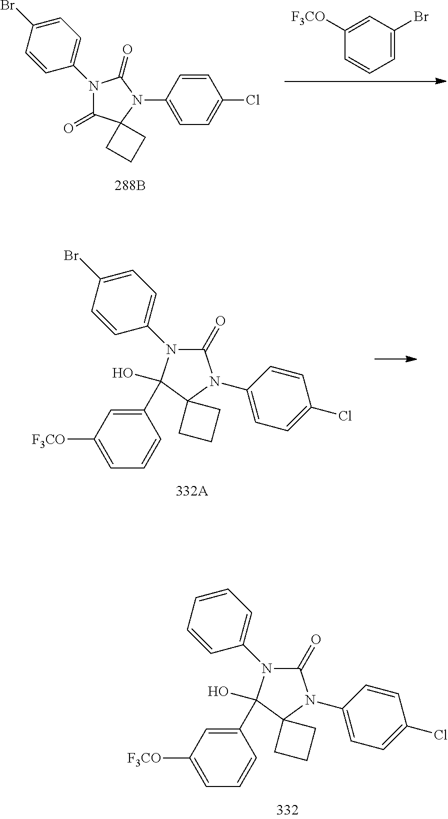 [SMILES: O=C1N(c2ccc(Br)cc2)C(=O)C2(CCC2)N1c1ccc(Cl)cc1 ; FC(F)(F)Oc1cccc(Br)c1 ; O=C1N(c2ccc(Cl)cc2)C2(CCC2)C(O)(c2cccc(OC(F)(F)F)c2)N1c1ccc(Br)cc1 ; O=C1N(c2ccc(Cl)cc2)C2(CCC2)C(O)(c2cccc(OC(F)(F)F)c2)N1c1ccccc1]

Compound 332A was synthesized by employing the procedure described for Compound 125 using Compound 288B and 1-bromo-3-(trifluoromethoxy)benzene in lieu of Compound 2C and 3-bromo-N,N-dimethylaniline. Compound 332A. LC-MS (ESI) m/z: 567 [M+H]⁺.

To a solution of Compound 332A (100 mg, 0.176 mmol) in THF (5 mL) was added a solution of t-BuLi in hexane (1.3 M, 0.81 mL, 1.056 mmol) at −60° C. under nitrogen. The reaction mixture was stirred at room temperature for 5 minutes, quenched with saturated NH₄Cl solution (10 mL), and extracted with ethyl acetate (5 mL×2). The combined organic layers was dried over anhydrous sodium sulfate, filtered, and concentrated. The residue was purified by preparative TLC (petroleum ether in ethyl acetate, 20% v/v) to afford Compound 332. LC-MS (ESI) m/z: 489 [M+H]⁺; ¹H-NMR (CDCl₃, 400 MHz): δ (ppm) 0.90-0.99 (m, 1H), 1.08-1.17 (m, 1H), 1.75-1.81 (m, 1H), 1.99-2.06 (m, 1H), 2.24-2.30 (m, 1H), 2.72-2.77 (m, 1H), 3.75-3.84 (m, 1H), 7.06-7.09 (m, 1H), 7.17-7.25 (m, 4H), 7.28-7.29 (m, 1H), 7.33-7.47 (m, 7H).

### Example 333

### Synthesis of 5-(4-chlorophenyl)-8-hydroxy-7-(p-tolyl)-8-(3-(trifluoromethoxy)phenyl)-5,7-diazaspiro[3.4]octan-6-one

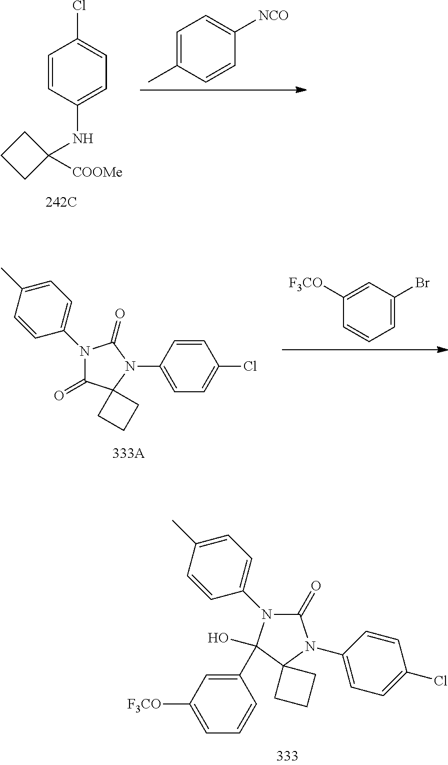

Compounds 333A and 333 were synthesized by employing the procedures described for Compounds 224D and 125 using 1-isocyanato-4-methylbenzene, Compounds 242C, 333A, and 1-bromo-3-(trifluoromethoxy)benzene in lieu of 1-chloro-4-isocyanatobenzene, Compounds 224C, 2C, and 3-bromo-N,N-dimethylaniline. Compound 333A. LC-MS (ESI) m/z: 341 [M+H]⁺. Compound 333. LC-MS (ESI) m/z: 503 [M+H]⁺. ¹H-NMR (400 MHz, CDCl₃): δ (ppm) 0.87-0.90 (m, 1H), 1.10-1.11 (m, 1H), 1.76-1.77 (m, 1H), 1.99-2.00 (m, 1H), 2.24 (m, 4H), 2.78-2.79 (m, 1H), 3.72 (s, 1H), 6.99-7.02 (m, 2H), 7.16-7.18 (m, 1H), 7.25-7.29 (m, 4H), 7.33-7.37 (m, 1H), 7.40-7.48 (m, 4H).

### Example 334

### Synthesis of 7-(4-chlorophenyl)-8-hydroxy-5-phenyl-8-(3-(trifluoromethoxy)phenyl)-5,7-diazaspiro[3.4]octan-6-one

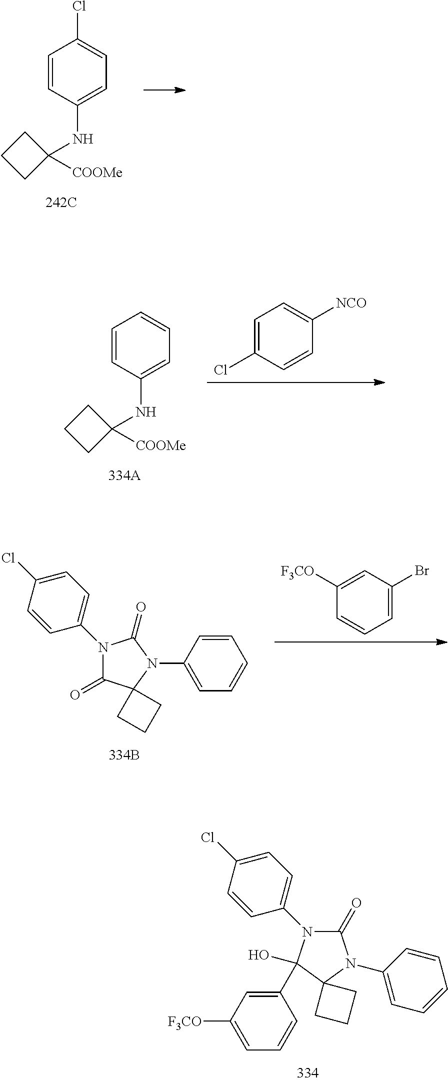

To a solution of Compound 242C (1.0 g, 4.2 mmol) in methanol (20 mL) was added Pd/C (10%, 150 mg). After the mixture was stirred at room temperature under hydrogen (1 atm) overnight, it was filtered through a pad of Celite. The filtrate was concentrated and purified with column chromatography on silica gel (ethyl acetate in petroleum ether, from 10% to 40% v/v) to furnish Compound 334A. LC-MS (ESI) m/z: 206 [M+H]⁺.

Compounds 334B and 334 were synthesized by employing the procedures described for Compounds 224D and 125 using Compounds 334A, 334B, and 1-bromo-3-(trifluoromethoxy)benzene in lieu of Compounds 224C, 2C, and 3-bromo-N,N-dimethylaniline. Compound 334B. LC-MS (ESI) m/z: 327 [M+H]⁺. Compound 334. LC-MS (ESI) m/z: 489 [M+H]⁺. ¹H-NMR (CDCl₃, 400 MHz): δ (ppm) 0.80-0.92 (m, 1H), 1.06-1.16 (m, 1H), 1.74-1.82 (m, 1H), 2.04-2.14 (m, 1H), 2.23-2.36 (m, 1H), 2.68-2.78 (m, 1H), 3.57 (s, 1H), 7.13-7.22 (m, 3H), 7.32-7.52 (m, 10H).

### Example 335

### Synthesis of 4-(8-hydroxy-6-oxo-5-phenyl-8-(3-(trifluoromethoxy)phenyl)-5,7-diazaspiro[3.4]octan-7-yl)benzonitrile

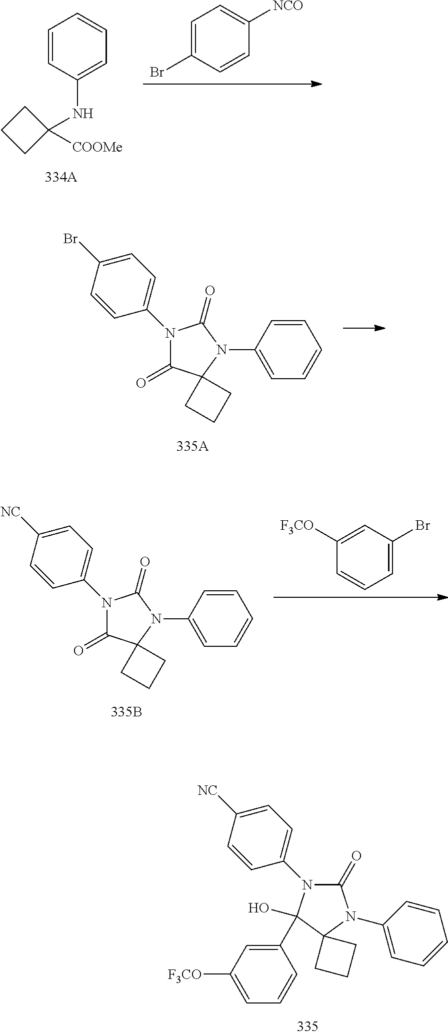

Compounds 335A, 335B, and 335 were synthesized by employing the procedures described for Compounds 224D, 180C, and 125 using 1-bromo-4-isocyanatobenzene, Compounds 334A, 335A, 335B, and 1-bromo-3-(trifluoromethoxy)benzene in lieu of Compounds 224C, 180B, 2C, and 3-bromo-N,N-dimethylaniline. Compound 335A. LC-MS (ESI) m/z: 372 [M+H]⁺. Compound 335B. LC-MS (ESI) m/z: 318 [M+H]⁺. Compound 335. LC-MS (ESI) m/z: 480 [M+H]⁺; ¹H-NMR (CD₃OD, 400 MHz): δ (ppm) 0.98 (m, 1H), 1.07 (m, 1H), 1.73 (m, 1H), 2.12 (m, 1H), 2.31 (m, 1H), 2.85 (m, 1H), 7.25 (m, 1H), 7.45-7.58 (m, 10H), 7.71 (m, 2H).

### Example 336

### Synthesis of 4-(8-hydroxy-6-oxo-7-phenyl-8-(3-(trifluoromethoxy)phenyl)-5,7-diazaspiro[3.4]octan-5-yl)benzonitrile

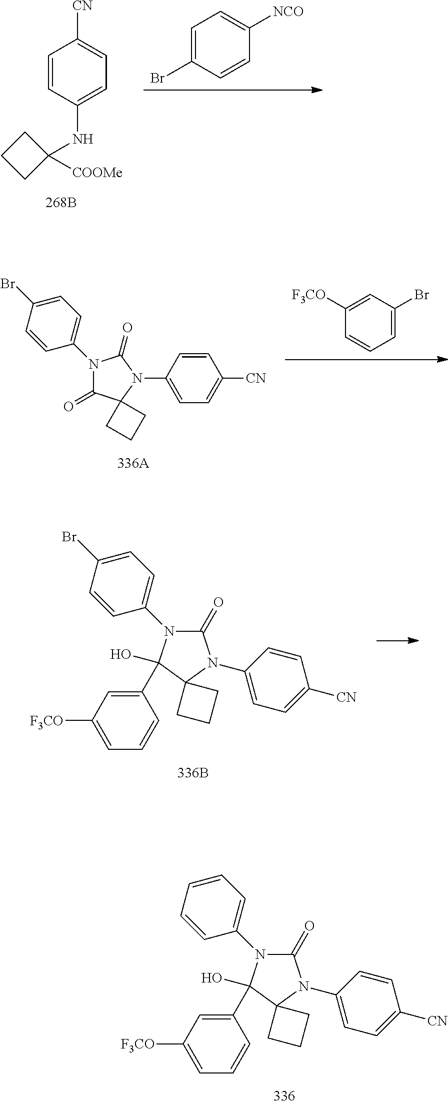

Compounds 336A, 336B, and 336 were synthesized by employing the procedures described for Compounds 224D, 125, and 332 using 1-bromo-4-isocyanatobenzene, Compounds 268B, 336A, 1-bromo-3-(trifluoromethoxy)benzene, and 336B in lieu of Compounds 224C, 2C, 3-bromo-N,N-dimethylaniline, and 332A. Compound 336A. LC-MS (ESI) m/z: 396 [M+H]⁺. Compound 336B. LC-MS (ESI) m/z: 558 [M+H]⁺. ¹H-NMR (CDCl₃, 400 MHz): δ (ppm) 0.86-0.95 (m, 1H), 1.17-1.28 (m, 1H), 1.81-1.88 (m, 1H), 2.00-2.08 (m, 1H), 2.29-2.37 (m, 1H), 2.74-2.82 (m, 1H), 3.74 (s, 1H), 7.17-7.48 (m, 10H), 7.73-7.75 (m, 2H). Compound 336. LC-MS (ESI) m/z: 480 [M+H]⁺. ¹H-NMR (CDCl₃, 400 MHz): δ (ppm) 0.88-0.98 (m, 1H), 1.16-1.26 (m, 1H), 1.82-1.88 (m, 1H), 2.00-2.10 (m, 1H), 2.27-2.38 (m, 1H), 2.74-2.83 (m, 1H), 3.64-3.87 (m, 1H), 7.09-7.13 (m, 1H), 7.18-7.25 (m, 3H), 7.34-7.39 (m, 3H), 7.44-7.49 (m, 4H), 7.67-7.73 (m, 2H).

### Example 337

### Synthesis of 4-(7-(4-ethylphenyl)-8-hydroxy-6-oxo-8-(3-(trifluoromethoxy)phenyl)-2-oxa-5,7-diazaspiro[3.4]octan-5-yl)benzonitrile

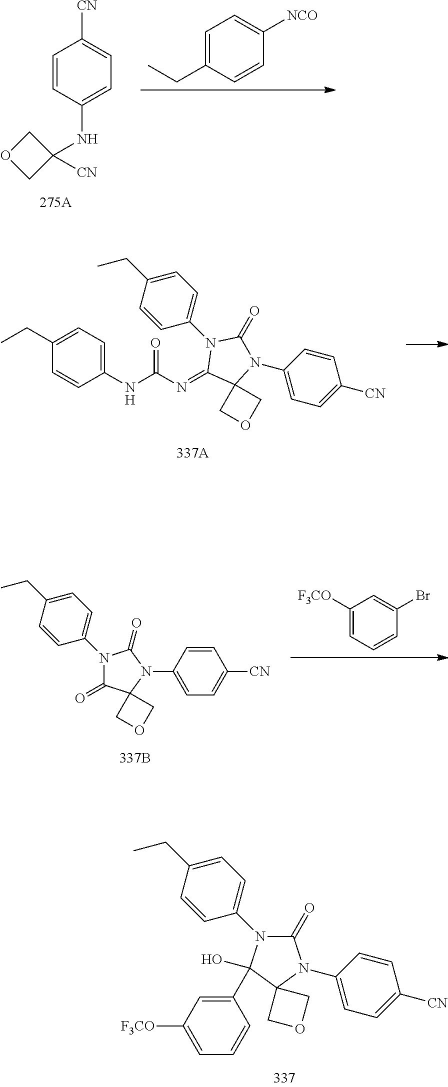

Compounds 337A, 337B, and 337 were synthesized by employing the procedures described for Compounds 271C, 271D, and 125 using 1-ethyl-4-isocyanatobenzene, Compounds 275A, 337A, 337B, and 1-bromo-3-(trifluoromethoxy)benzene in lieu of 1-chloro-4-isocyanatobenzene, Compounds 271B, 271C, 2C, and 3-bromo-N,N-dimethylaniline. Compound 337A. LC-MS (ESI) m/z: 494 [M+H]⁺; ¹H-NMR (DMSO-d₆, 400 MHz): δ (ppm) 1.07 (t, J=8.0 Hz, 3H), 1.13 (t, J=8.0 Hz, 3H), 2.48-2.54 (m, 4H), 4.89 (d, J=8.0 Hz, 2H), 5.13 (d, J=8.0 Hz, 2H), 7.01 (d, J=8.0 Hz, 2H), 7.06-7.08 (m, 2H), 7.13 (d, J=8.0 Hz, 2H), 7.27 (d, J=8.0 Hz, 2H), 7.99 (d, J=8.8 Hz, 2H), 8.05 (d, J=8.8 Hz, 2H), 9.38 (s, 1H). Compound 337B. LC-MS (ESI) m/z: 348 [M+H]⁺; ¹H-NMR (DMSO-d₆, 400 MHz): δ (ppm) 1.21 (t, J=7.2 Hz, 3H), 2.66 (q, J=7.2 Hz, 2H), 4.81 (d, J=8.4 Hz, 2H), 4.89 (d, J=8.4 Hz, 2H), 7.33-7.37 (m, 4H), 7.99 (d, J=8.8 Hz, 2H), 8.05 (d, J=8.8 Hz, 2H). Compound 337. LC-MS (ESI) m/z: 510 [M+H]⁺; ¹H-NMR (CDCl₃, 400 MHz): δ (ppm) 1.17 (t, J=7.6 Hz, 3H), 2.66 (q, J=7.6 Hz, 2H), 4.18 (d, J=8.0 Hz, 1H), 4.24 (s, 1H), 4.66 (d, J=8.0 Hz, 1H), 4.73 (d, J=8.0 Hz, 1H), 5.24 (d, J=8.0 Hz, 1H), 7.06 (d, J=8.4 Hz, 2H), 7.17 (d, J=8.4 Hz, 2H), 7.26 (brs, 1H), 7.35 (s, 1H), 7.44 (d, J=4.8 Hz, 2H), 7.63 (d, J=8.4 Hz, 2H), 7.75 (d, J=8.8 Hz, 2H).

### Example 338

### Synthesis of 5-(4-chlorophenyl)-8-hydroxy-7-phenyl-8-(3-(trifluoromethoxy)phenyl)-2-oxa-5,7-diazaspiro[3.4]octan-6-one

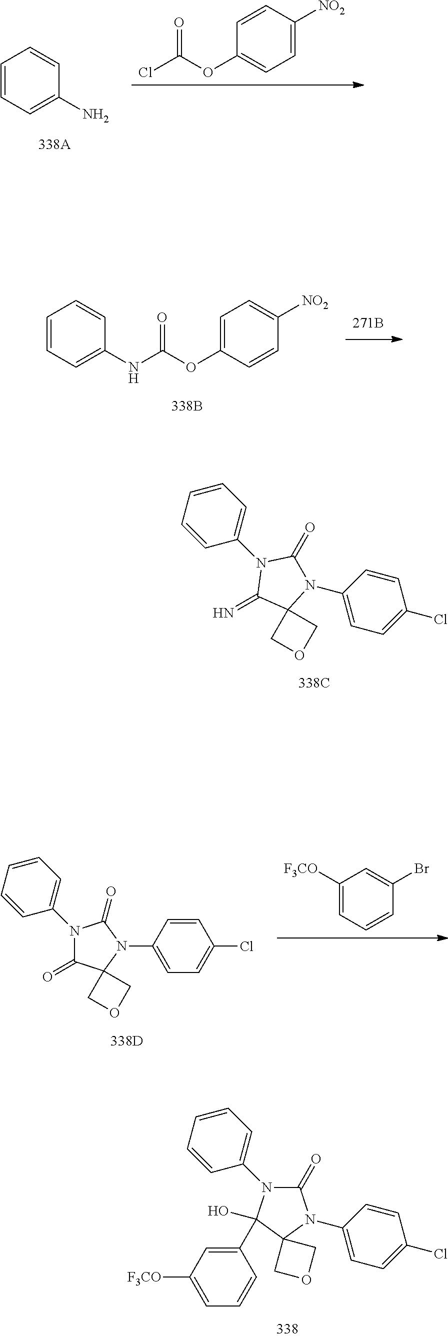

To a mixture of aniline 338A (1.86 g, 20 mmol) and triethylamine (3.0 g, 30 mmol) in dichloromethane (100 mL) was added 4-nitrophenyl carbonochloridate (4.0 g, 20 mmol) at −20° C. The mixture was stirred at −20° C. for 1 hour, washed with water (50 mL) and brine (50 mL), dried over anhydrous sodium sulfate, filtered, and concentrated to give a crude Compound 338B. LC-MS (ESI) m/z: non-ionizable compound under routine conditions used.

To a solution of Compound 338B (2.0 g, 7.5 mmol) in anhydrous pyridine (20 mL) was added Compound 271B (1.0 g, 5.0 mmol) at 90° C. and stirred at 90° C. for 16 hours. The reaction mixture was evaporated to remove most of pyridine. The residue was slurried in Et₂O (10 mL), filtered, and dried under vacuum to afford Compound 338C. LC-MS (ESI) m/z: 328 [M+H]⁺.

Compounds 338D and 338 were synthesized by employing the procedures described for Compounds 271D and 125 using Compounds 338C, 338D, and 1-bromo-3-(trifluoromethoxy)benzene in lieu of Compounds 271C, 2C, and 3-bromo-N,N-dimethylaniline. Compound 338D. LC-MS (ESI) m/z: 329 [M+H]⁺; ¹H-NMR (DMSO-d₆, 400 MHz): (ppm) 4.70 (d, J=7.6 Hz, 2H), 4.86 (d, J=7.6 Hz, 2H), 7.46-7.52 (m, 5H), 7.65-7.68 (m, 4H). Compound 338. LC-MS (ESI) m/z: 491 [M+H]⁺; ¹H-NMR (DMSO-d₆, 400 MHz): δ (ppm) 3.79 (d, J=8.0 Hz, 1H), 4.43 (d, J=8.4 Hz, 1H), 4.55 (d, J=8.4 Hz, 1H), 5.09 (d, J=8.4 Hz, 1H), 7.03-7.05 (m, 1H), 7.18-7.21 (m, 2H), 7.31-7.33 (m, 1H), 7.42-7.44 (m, 2H), 7.50-7.54 (m, 2H), 7.60-7.66 (m, 5H), 7.80 (s, 1H).

### Preparation of Compounds of Formula VII

The following are illustrative examples of how the compounds of Formula VII, and/or stereoisomers thereof, can be prepared.

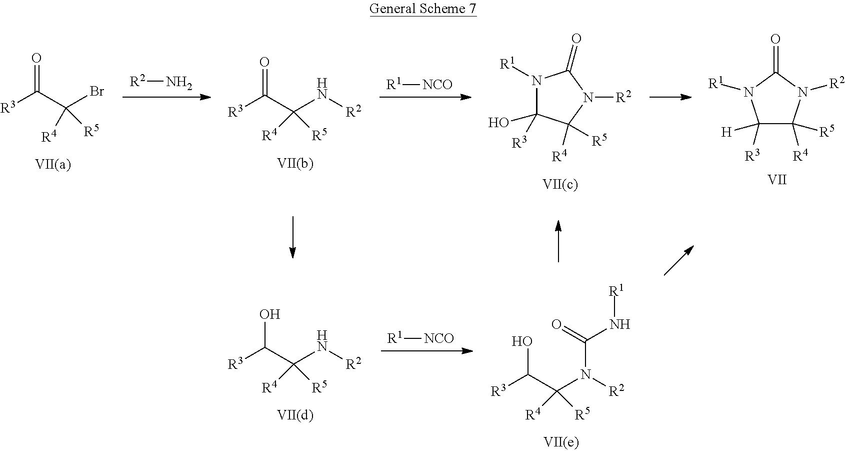

A compound of Formula VII can be prepared according to General Scheme 7. The intermediate of formula VII(c) can be prepared according to various procedures disclosed herein or are known to one of ordinary skill in the art.

The intermediate of formula VII(b) can be prepared using standard nucleophilic amines substitution conditions. More specifically, an intermediate of formula VII(a) can be treated with an amine of formula R²—NH₂ in a solvent such as EtOH, NMP, DMSO, or DMF, optionally in the presence of a base such as carbonate, bicarbonate, DIPEA, or TEA.

The intermediate of formula VII(b) can be treated with an isocyanate of formula R¹—NCO in a solvent such as DCM, toluene, or pyridine, optionally in the presence of a base such as DIPEA or TEA, to yield the intermediate of formula VII(c).

The intermediate of formula VII(c) can also be made by an alternative method from the intermediate of formula VII(b) through the intermediates of formulas VII(d) and VII(e).

The intermediate of formula VII(b) can be reduced to the corresponding alcohol by using standard reduction conditions from ketone to alcohol. More specifically, the intermediate of formula VII(b) can be treated with a reducing agent such as sodium borohydride, in a solvent such as EtOH, or THF to yield an intermediate of formula VII(d).

The intermediate of formula VII(d) can be treated with an isocyanate of formula R¹—NCO in a solvent such as DCM, toluene, or pyridine, optionally in the presence of a base such as DIPEA or TEA, to yield an intermediate of formula VII(e).

The intermediate of formula VII(c) can be prepared using standard oxidation conditions for alcohol to ketone. More specifically, the intermediate of formula VII(e) can be treated with an oxidizing reagent such as Dess-Martin periodinane (DMP) in a solvent such as DCM or CHCl₃, in the presence of a base such as sodium bicarbonate.

The compound of Formula VII can be prepared using standard dehydroxylation conditions for removing hydroxyl group. More specifically, the intermediate of formula VII(c) can be treated with triethylsilane (Et₃SiH) in a solvent such as CH₂Cl₂ or CHCl₃, in the presence of an acid such as boron trifluoride diethyl etherate or 2,2,2-trifluoroacetic acid at an appropriate temperature, preferably at −20-40° C.

The compound of Formula VII can also be prepared using Mitsunobu reaction conditions. More specifically, the intermediate of formula VII(e) can be treated with an azodicarboxylate such as diethyl azodicarboxylate (DEAD) or diisopropyl azodicarboxylate (DIAD) in the presence of triphenylphosphine and in a solvent such as toluene or THF, at an appropriate temperature, preferably at 40-130° C.

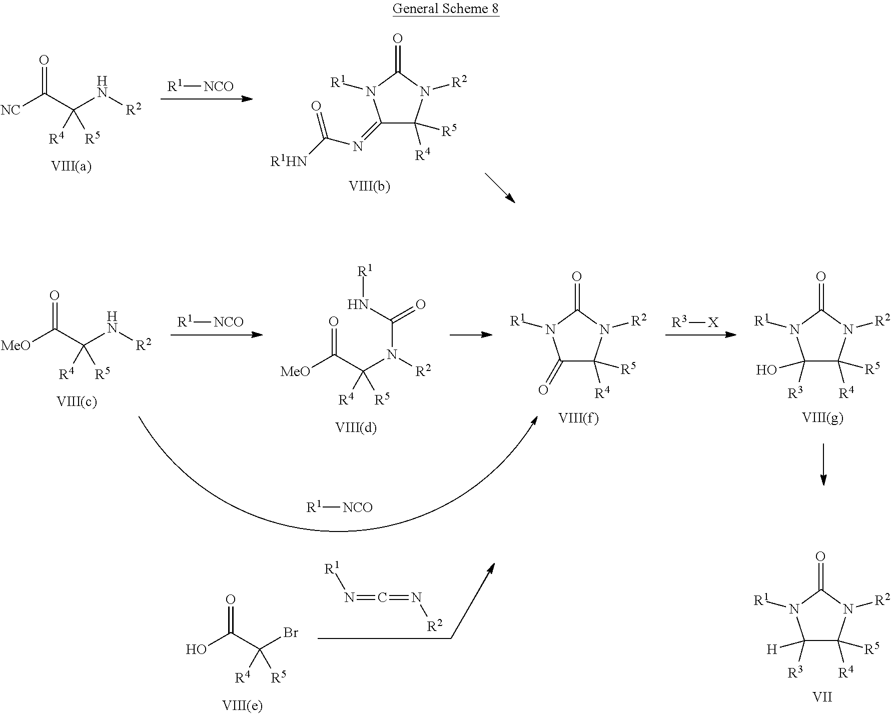

The compound of Formula VII can also be prepared according to General Scheme 8. The intermediate of formula VIII(f) can be prepared according to various procedures disclosed herein or are known to one of ordinary skill in the art.

The intermediate of formula VIII(b) can be prepared using standard urea formation conditions. More specifically, the intermediate of formula VIII(a) can be treated with an isocyanate of formula R¹—NCO in a solvent such as DCM, toluene, or pyridine, optionally in the presence of a base such as DIPEA or TEA, to yield the intermediate of formula VIII(b).

The intermediate of formula VIII(b) can be treated under acidic conditions in a solvent such as methanol and/or THF, to yield the intermediate of formula VIII(f).

The intermediate of formula VIII(f) can also be made by an alternative method from formula VIII(c) through the intermediate of formula VIII(d), or directly from formula VIII(c).

The intermediate of formula VIII(d) can be prepared using standard urea formation conditions. More specifically, the intermediate of formula VIII(c) can be treated with an isocyanate of formula R¹—NCO in a solvent such as DCM, toluene, or pyridine, optionally in the presence of a base such as DIPEA or TEA, to yield the intermediate of formula VIII(d).

The intermediate of formula VIII(d) can be treated with ammonia in a solvent such as methanol or ethanol, to yield the intermediate of formula VIII(f).

The intermediate of formula VIII(c) can be treated under the standard urea formation conditions, to directly yield the intermediate of formula VIII(f).

The intermediate of formula VIII(f) can also be made by another alternative method from formula VIII(e).

The intermediate of formula VIII(f) can be prepared using hydantoin formation conditions. More specifically, the intermediate of formula VIII(e) can be treated with an carbodiimide of formula R¹—N═C═N—R² in a solvent such as dioxane, DCM, THF, or pyridine, optionally in the presence of a base such as DIPEA or TMP, to yield the intermediate of formula VIII(f).

The intermediate of formula VIII(g) can be prepared using standard nucleophilic addition conditions. More specifically, the intermediate of formula VIII(f) can be treated with a generated organolithium reagent of formula R³Li from corresponding halide reagent of formula R³—X in a solvent such as THF, or ether, to yield the intermediate of formula VIII(g).

The intermediate of formula VIII(g) can also be treated with thionyl chloride (SOCl₂) or triethylsilane in a solvent such as toluene or DCM or CHCl₃, optionally in the presence of an acid such as boron trifluoride diethyl etherate or 2,2,2-trifluoroacetic acid at an appropriate temperature, preferably at −20-40° C., to afford the compound of Formula VII.

### SYNTHETIC EXAMPLES (COMPOUNDS OF FORMULA VII)

### Example 1

### Synthesis of 1,3-bis(4-bromophenyl)octahydro-2H-benzo[d]imidazol-2-one (1)

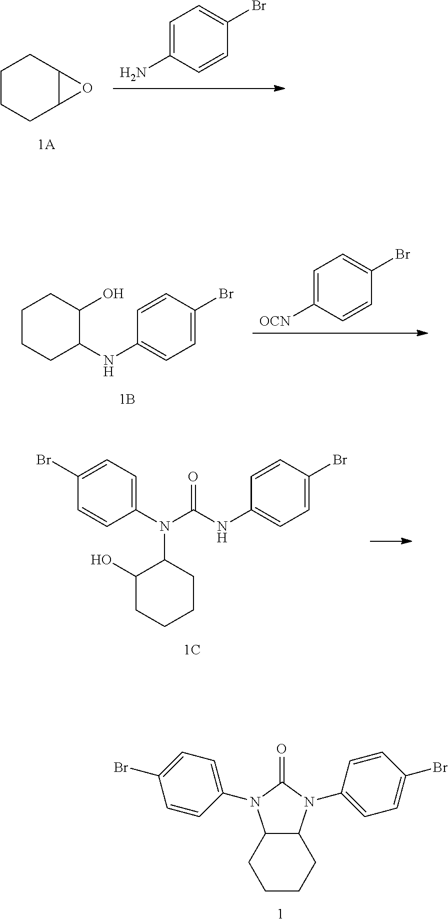

A solution of 4-bromoaniline (3.5 g, 20.4 mmol) and 7-oxabicyclo[4.1.0]heptane 1A (2 g, 20.4 mmol) in methanol (30 mL) was heated at reflux overnight. After evaporation of solvent, the residue was purified with flash column chromatography on silica gel (ethyl acetate in petroleum ether, from 0% to 20% v/v) to yield Compound 1B. LC-MS (ESI) m/z: 270 [M+H]⁺; ¹H-NMR (CDCl₃, 400 MHz): δ (ppm) 1.00-1.10 (m, 1H), 1.25-1.45 (m, 3H), 1.71-1.80 (m, 2H), 2.05-2.13 (m, 2H), 2.55 (s, 1H), 3.06-3.12 (m, 1H), 3.33-3.40 (m, 2H), 6.57-6.61 (m, 2H), 7.23-7.27 (m, 2H).

A solution of 1-bromo-4-isocyanatobenzene (1.1 g, 5.58 mmol) and Compound 1B (1.5 g, 5.58 mmol) in dichloromethane (30 mL) was stirred at room temperature overnight. The reaction mixture was concentrated and purified with flash column chromatography on silica gel (ethyl acetate in petroleum ether, from 0% to 20% v/v) to yield Compound 1C. LC-MS (ESI) m/z: 467 [M+H]⁺; ¹H-NMR (CDCl₃, 400 MHz): δ (ppm) 0.93-1.10 (m, 2H), 1.24-1.44 (m, 2H), 1.65-1.70 (m, 2H), 1.80-1.84 (m, 1H), 2.08-2.11 (m, 1H), 2.56-2.60 (m, 1H), 3.21-3.29 (m, 1H), 4.37-4.44 (m, 1H), 5.91 (s, 1H), 7.12-7.15 (m, 2H), 7.24-7.28 (m, 2H), 7.31-7.35 (m, 2H), 7.63-7.65 (m, 2H).

To a mixture of Compound 1C (150 mg, 0.32 mmol) and PPh₃ (168 mg, 0.64 mmol) in anhydrous toluene (15 mL) was added DIAD (129 mg, 0.64 mmol) at 25° C., heated at 110° C. under nitrogen for 16 hours, and concentrated under reduced pressure. The residue was purified with preparative HPLC to furnish Compound 1. LC-MS (ESI) m/z: 449 [M+H]⁺; ¹H-NMR (DMSO-d₆, 400 MHz): δ (ppm) 1.35 (s, 4H), 1.55-1.58 (m, 2H), 1.82-1.85 (m, 2H), 4.47 (s, 2H), 7.47 (d, J=8.8 Hz, 4H), 5.56 (d, J=8.8 Hz, 4H).

### Example 2

### Synthesis of 1,3-bis(4-bromophenyl)-4-methyl-5-phenylimidazolidin-2-one(2), (4S,5S)-1,3-bis(4-bromophenyl)-4-methyl-5-phenylimidazolidin-2-one (2-1), (4R,5R)-1,3-bis(4-bromophenyl)-4-methyl-5-phenylimidazolidin-2-one (2-2), (4S,5R)-1,3-bis(4-bromophenyl)-4-methyl-5-phenylimidazolidin-2-one (2-3), and (4R,5S)-1,3-bis(4-bromophenyl)-4-methyl-5-phenylimidazolidin-2-one (2-4)

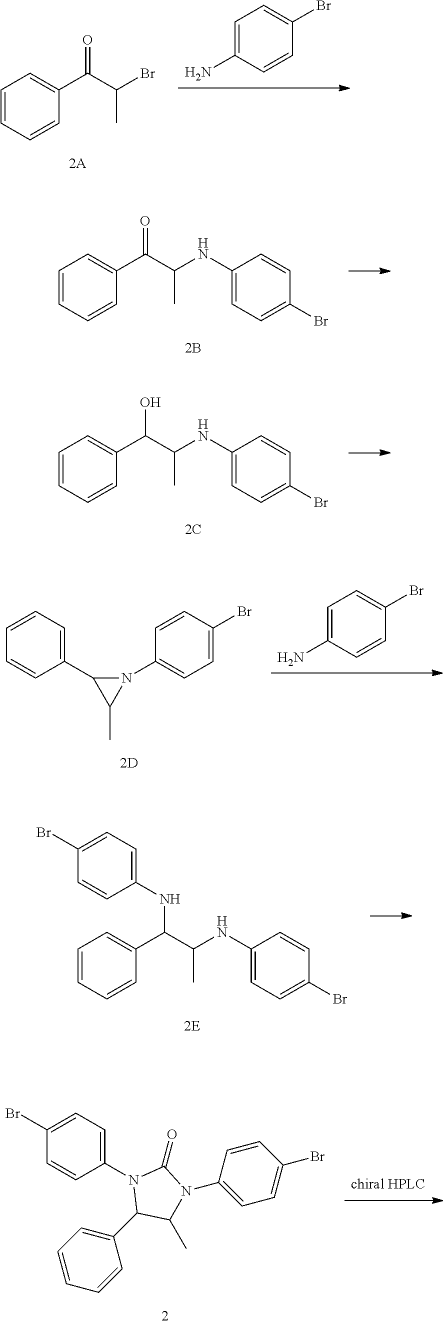

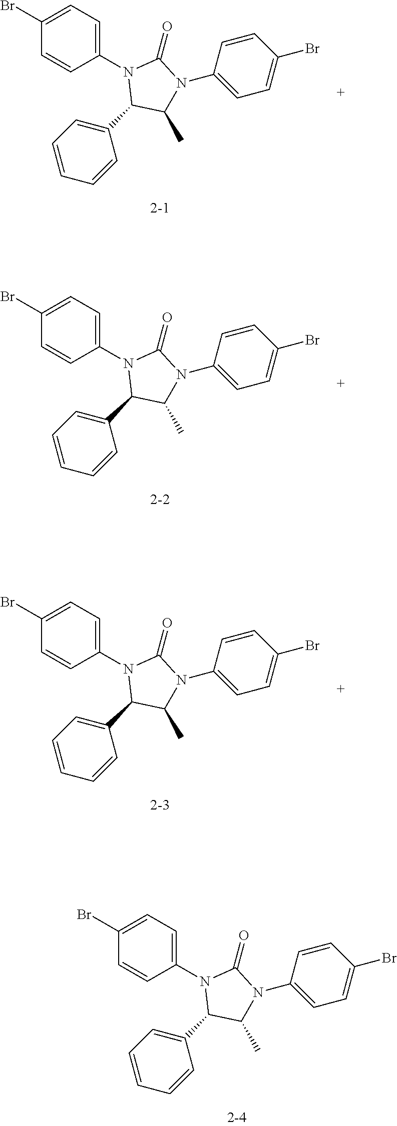 [SMILES: C[C@H]1[C@H](c2ccccc2)N(c2ccc(Br)cc2)C(=O)N1c1ccc(Br)cc1 ; C[C@@H]1[C@@H](c2ccccc2)N(c2ccc(Br)cc2)C(=O)N1c1ccc(Br)cc1 ; C[C@H]1[C@@H](c2ccccc2)N(c2ccc(Br)cc2)C(=O)N1c1ccc(Br)cc1 ; C[C@@H]1[C@H](c2ccccc2)N(c2ccc(Br)cc2)C(=O)N1c1ccc(Br)cc1]

To a mixture of 4-bromoaniline (1.61 g, 9.43 mmol) and NaHCO₃ (1.58 g, 18.9 mmol) in NMP (60 mL) was added 2-bromo-1-phenylpropan-1-one 2A (2.0 g, 9.43 mmol) and heated at 60° C. overnight. After cooling down to room temperature, the mixture was diluted with water (50 mL) and concentrated to remove most of solvent. The residue was extracted with ethyl acetate (50 mL×3). The combined extracts were washed with water (30 mL) and brine (30 mL), dried over anhydrous sodium sulfate, filtered, and concentrated to give a crude product, which was purified with flash column chromatography on silica gel (ethyl acetate in petroleum ether, 10% v/v) to yield Compound 2B. LC-MS (ESI) m/z: 304 [M+H]⁺; ¹H-NMR (CDCl₃, 400 MHz): δ (ppm) 1.48 (d, J=6.8 Hz, 3H), 5.06-5.11 (m, 1H), 6.46-6.60 (m, 3H), 7.24 (d, J=8.4 Hz, 2H), 7.54 (d, J=8.0 Hz, 2H), 7.64 (t, J=8.0 Hz, 1H), 8.01 (d, J=8.0 Hz, 2H).

To a solution of Compound 2B (1.95 g, 6.44 mmol) in methanol (60 mL) was added NaBH₄ (245 mg, 6.44 mmol) and the mixture was stirred at room temperature for 2 hours. The reaction mixture was quenched with water (10 mL) and concentrated to remove most of solvent. The residue was diluted with ethyl acetate (150 mL), washed with water (10 mL) and brine (10 mL), dried over anhydrous sodium sulfate, filtered, and concentrated to give a crude Compound 2C. LC-MS (ESI) m/z: 306 [M+H]⁺.

To a solution of Compound 2C (1.38 g, 4.52 mmol)) in dichloromethane (50 mL) at 0° C. was added triethylamine (1.37 g, 13.56 mmol) and methanesulfonyl chloride (518 mg, 4.52 mmol). The mixture was stirred at room temperature overnight, diluted with water (10 mL), and extracted with dichloromethane (50 mL×3). The combined extracts were washed with water (10 mL) and brine (10 mL), dried over anhydrous sodium sulfate, filtered, and concentrated to give a crude product, which was purified with flash column chromatography on silica gel (ethyl acetate in petroleum ether, 10% v/v) to yield Compound 2D. LC-MS (ESI) m/z: 288 [M+H]⁺; ¹H-NMR (CDCl₃, 400 MHz): δ (ppm) 1.14, 1.24 (d, J=6.0 Hz, 3H), 2.49-2.55, 2.60-2.65 (m, 1H), 2.92-2.93, 3.27-3.28 (m, 1H), 6.81, 6.93 (d, J=8.4 Hz, 2H), 7.28-7.42 (m, 7H).

To a solution of Compound 2D (850 mg, 2.95 mmol) in dichloromethane (30 mL) was added 4-bromoaniline (600 mg, 2.95 mmol) and triethylamine (596 mg, 5.9 mmol). The mixture was stirred at room temperature overnight, quenched with water (10 mL), and extracted with dichloromethane (50 mL×3). The combined extracts were washed with water (10 mL) and brine (10 mL), dried over anhydrous sodium sulfate, filtered, and concentrated to give a crude product, which was purified with flash column chromatography on silica gel (ethyl acetate in petroleum ether, 10% v/v) to yield Compound 2E. LC-MS (ESI) m/z: 459 [M+H]⁺.

To a solution of Compound 2E (675 mg, 1.47 mmol) in dichloromethane (20 mL) was added triethylamine (297 mg, 2.94 mmol) and triphosgene (438 mg, 1.47 mmol). The mixture was stirred at room temperature for 2 hours and concentrated under reduced pressure. The residue was purified with preparative HPLC to furnish Compound 2, which was further separated using chiral HPLC to afford Compound 2-1, Compound 2-2, Compound 2-3, and Compound 2-4.

Compound 2-1: LC-MS (ESI) m/z: 485 [M+H]⁺; ¹H-NMR (CDCl₃, 400 MHz): δ (ppm) 0.81 (d, J=6.0 Hz, 3H), 4.62-4.69 (m, 1H), 5.36 (d, J=8.8 Hz, 1H), 7.25 (d, J=7.6 Hz, 2H), 7.31-7.37 (m, 9H), 7.51 (d, J=8.4 Hz, 2H). Chiral separation conditions: n-hexane/EtOH contained 0.1% DEA (50/50); S,S-WHELK-Ol (4.6×250 mm, 5 μm); retention time: 13.12 minutes.

Compound 2-2: LC-MS (ESI) m/z: 485 [M+H]⁺; ¹H-NMR (CDCl₃, 400 MHz): δ (ppm) 0.81 (d, J=6.4 Hz, 3H), 4.62-4.69 (m, 1H), 5.35 (d, J=8.8 Hz, 1H), 7.25 (d, J=7.6 Hz, 2H), 7.31-7.37 (m, 9H), 7.51 (d, J=8.8 Hz, 2H). Chiral separation conditions: n-hexane/EtOH contained 0.1% DEA (50/50); S,S-WHELK-Ol (4.6×250 mm, 5 μm); retention time: 10.59 minutes.

Compound 2-3: LC-MS (ESI) m/z: 485 [M+H]⁺; ¹H-NMR (CDCl₃, 400 MHz): δ (ppm) 1.44 (d, J=6.0 Hz, 3H), 4.06-4.12 (m, 1H), 4.79 (d, J=5.6 Hz, 1H), 7.31-7.38 (m, 11H), 7.48 (d, J=8.4 Hz, 2H). Chiral separation conditions: co-solvent: n-hexane/EtOH contained 0.1% DEA (50/50); S,S-WHELK-Ol (4.6×250 mm, 5 μm); retention time: 19.88 minutes.

Compound 2-4: LC-MS (ESI) m/z: 485 [M+H]⁺; ¹H-NMR (CDCl₃, 400 MHz): δ (ppm) 1.46 (d, J=6.0 Hz, 3H), 4.08-4.14 (m, 1H), 4.80 (d, J=5.6 Hz, 1H), 7.32-7.39 (m, 11H), 7.49 (d, J=8.8 Hz, 2H). Chiral separation conditions: n-hexane/EtOH contained 0.1% DEA (50/50); S,S-WHELK-Ol (4.6×250 mm, 5 μm); retention time: 7.13 minutes.

An alternative synthetic method:

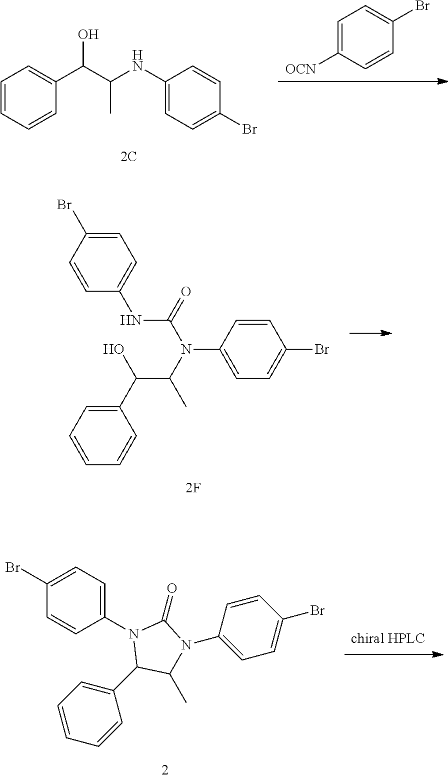

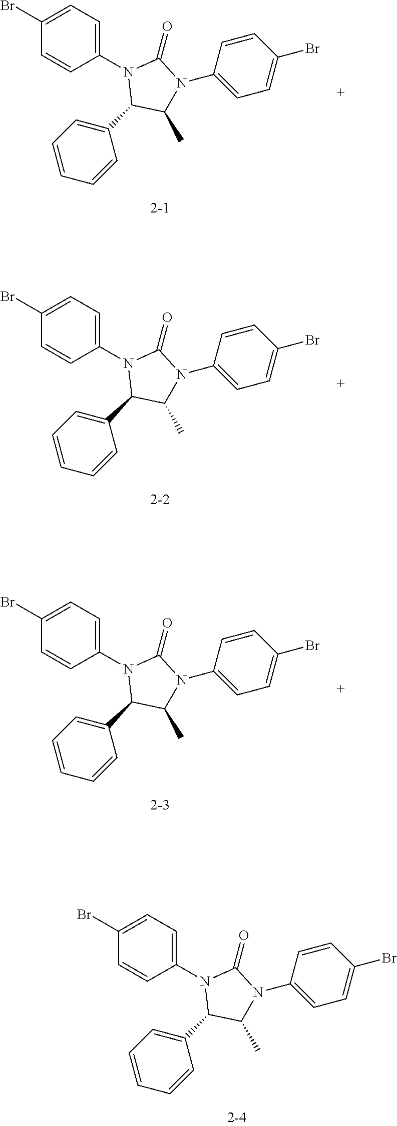 [SMILES: C[C@H]1[C@H](c2ccccc2)N(c2ccc(Br)cc2)C(=O)N1c1ccc(Br)cc1 ; C[C@@H]1[C@@H](c2ccccc2)N(c2ccc(Br)cc2)C(=O)N1c1ccc(Br)cc1 ; C[C@H]1[C@@H](c2ccccc2)N(c2ccc(Br)cc2)C(=O)N1c1ccc(Br)cc1 ; C[C@@H]1[C@H](c2ccccc2)N(c2ccc(Br)cc2)C(=O)N1c1ccc(Br)cc1]

Compounds 2F and 2 were synthesized by employing the procedures described for Compounds 1C and 1 using Compounds 2C and 2F in lieu of Compounds 1B and IC.

Compound 2F. LC-MS (ESI) m/z: 503 [M+H]⁺; ¹H-NMR (CDCl₃, 400 MHz): δ (ppm) 1.28 (d, J=7.6 Hz, 3H), 4.08-4.11 (m, 1H), 4.14 (s, 1H), 5.30 (s, 1H), 5.88 (s, 1H), 6.98 (d, J=8.0 Hz, 2H), 7.16 (d, J=8.0 Hz, 2H), 7.29-7.41 (m, 7H), 7.56 (d, J=8.0 Hz, 2H).

Compound 2 was separated with chiral HPLC to afford Compound 2-1, Compound 2-2, Compound 2-3, and Compound 2-4.

Compound 2-1: LC-MS (ESI) m/z: 485 [M+H]⁺; ¹H-NMR (CDCl₃, 400 MHz): δ (ppm) 0.81 (d, J=6.4 Hz, 3H), 4.62-4.69 (m, 1H), 5.36 (d, J=8.4 Hz, 1H), 7.25 (d, J=8.0 Hz, 2H), 7.32-7.37 (m, 9H), 7.51 (d, J=8.8 Hz, 2H). Chiral separation condition: n-hexane/EtOH contained 0.1% DEA (50/50); S,S-WHELK-Ol (4.6×250 mm, 5 μm); retention time: 12.87 minute.

Compound 2-2: LC-MS (ESI) m/z: 485 [M+H]⁺; ¹H-NMR (CDCl₃, 400 MHz): δ (ppm) 0.81 (d, J=6.4 Hz, 3H), 4.62-4.69 (m, 1H), 5.36 (d, J=8.8 Hz, 1H), 7.25 (d, J=8.0 Hz, 2H), 7.31-7.37 (m, 9H), 7.52 (d, J=6.8 Hz, 2H). Chiral separation condition: n-hexane/EtOH contained 0.1% DEA (50/50); S,S-WHELK-Ol (4.6×250 mm, 5 μm); retention time: 10.39 minute.

Compound 2-3: LC-MS (ESI) m/z: 485 [M+H]⁺; ¹H-NMR (CDCl₃, 400 MHz): δ (ppm) 1.45 (d, J=6.0 Hz, 3H), 4.08-4.14 (m, 1H), 4.79 (d, J=6.4 Hz, 1H), 7.33-7.36 (m, 11H), 7.49 (d, J=8.8 Hz, 2H). Chiral separation condition: MeOH contained 0.2% Methanol Ammonia; RegisCell (4.6×250 mm 5 μm); retention time: 3.56 minute.

Compound 2-4: LC-MS (ESI) m/z: 485 [M+H]⁺; ¹H-NMR (CDCl₃, 400 MHz): δ (ppm) 1.46 (d, J=6.0 Hz, 3H), 4.08-4.14 (m, 1H), 4.80 (d, J=6.0 Hz, 1H), 7.32-7.37 (m, 11H), 7.49 (d, J=8.8 Hz, 2H). Chiral separation condition: MeOH contained 0.2% Methanol Ammonia; RegisCell (4.6×250 mm 5 μm); retention time: 4.51 minute.

### Example 3

### Synthesis of 1,3-bis(4-chlorophenyl)-4-methyl-5-(m-tolyl)imidazolidin-2-one (3)

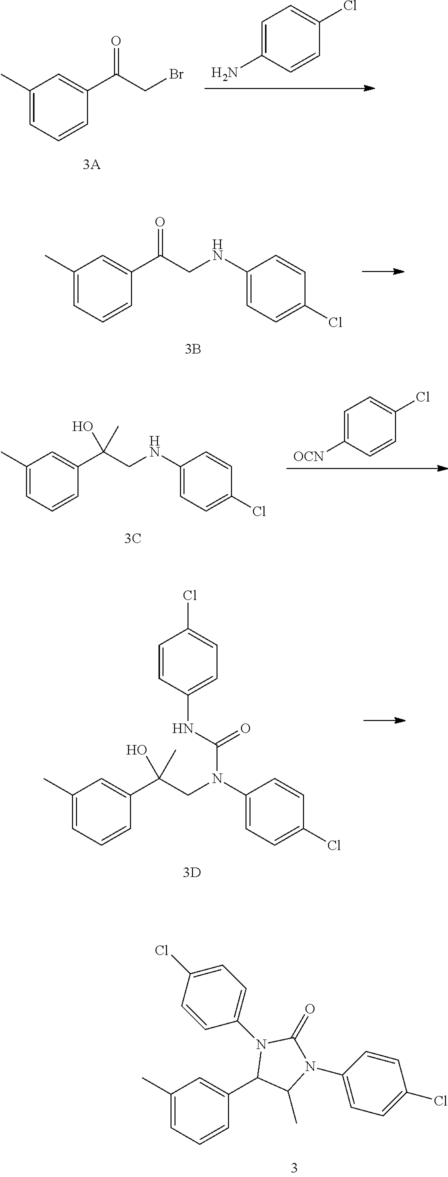

Compound 3B was synthesized by employing the procedure described for Compound 2B using 4-chloroaniline and Compound 3A using EtOH as solvent at room temperature in lieu of 4-bromoaniline and Compound 2A using NMP as solvent at 60° C. LC-MS (ESI) m/z: 260 [M+H]⁺.

To a solution of Compound 3B (900 mg, 3.47 mmol) in THF (5 mL) was dropped a solution of methylmagnesium bromide in THF (3 M, 1.4 mL, 4.16 mmol) at 25° C. under nitrogen. The mixture was stirred at 25° C. for 16 hours, quenched with saturated NH₄Cl solution (50 mL), and extracted with ethyl acetate (160 mL). The extract was washed with water (200 mL) and brine (150 mL), dried over anhydrous sodium sulfate, filtered, and evaporated to give a crude product, which was purified with flash column chromatography on silica gel (ethyl acetate in petroleum ether, 20% v/v) to afford Compound 3C. LC-MS (ESI) m/z: 276 [M+H]⁺.

Compounds 3D and 3 were synthesized by employing the procedures described for Compounds 1C and 1 using 1-chloro-4-isocyanatobenzene, Compounds 3C, and 3D in lieu of 1-bromo-4-isocyanatobenzene, Compounds 1B, and 1C.

Compound 3D. LC-MS (ESI) m/z: 429 [M+H]⁺; ¹H-NMR (CDCl₃, 400 MHz): δ (ppm) 1.52 (s, 3H), 2.24 (s, 3H), 3.78 (d, J=15.2 Hz, 1H), 4.28 (d, J=15.2 Hz, 1H), 5.71 (brs, 1H), 6.25 (brs, 1H), 6.67-6.69 (m, 2H), 7.01-7.26 (m, 10H).

Compound 3. LC-MS (ESI) m/z: 411 [M+H]⁺; ¹H-NMR (DMSO-d₆, 400 MHz): δ (ppm) 1.36 (d, J=6 Hz, 3H), 2.38 (s, 3H), 4.20-4.23 (m, 1H), 5.03-5.05 (m, 1H), 7.04-7.07 (m, 2H), 7.15-7.19 (m, 5H), 7.25-7.38 (m, 3H), 7.46-7.49 (m, 2H).

### Example 4

### Synthesis of 1,3-bis(4-chlorophenyl)-4-methyl-5-(3-(trifluoromethoxy) phenyl)imidazolidin-2-one (4), (4S,5S)-1,3-bis(4-chlorophenyl)-4-methyl-5-(3-(trifluoromethoxy)phenyl)imidazolidin-2-one (4-1), and (4R,5R)-1,3-bis(4-chlorophenyl)-4-methyl-5-(3-(trifluoromethoxy)phenyl)imidazolidin-2-one (4-2)

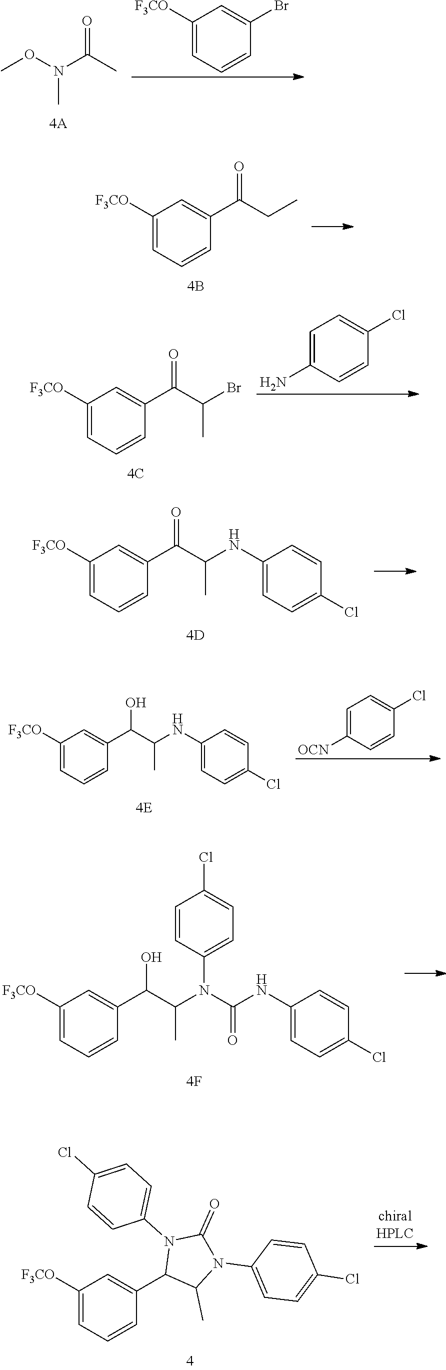

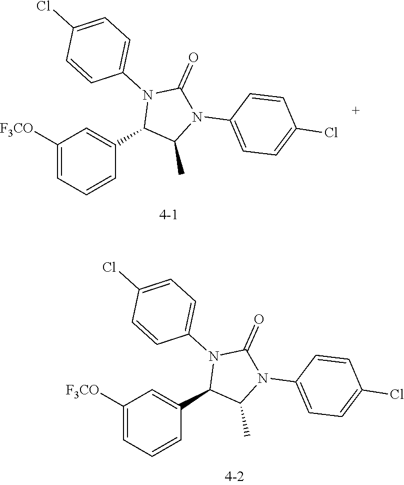 [SMILES: C[C@H]1[C@H](c2cccc(OC(F)(F)F)c2)N(c2ccc(Cl)cc2)C(=O)N1c1ccc(Cl)cc1 ; C[C@@H]1[C@@H](c2cccc(OC(F)(F)F)c2)N(c2ccc(Cl)cc2)C(=O)N1c1ccc(Cl)cc1]

To a solution of 1-bromo-3-(trifluoromethyl)benzene (2.00 g, 8.30 mmol) in THF (20 mL) was dropped a solution of n-BuLi in n-hexane (2.5 M, 3.3 mL, 8.30 mmol) at −78° C. under nitrogen and the mixture was stirred at −78° C. for 15 minutes. To the solution at −78° C. was dropped a solution of Compound 4A (1.17 g, 9.93 mmol) in THF (4 mL) and the mixture was stirred at the same temperature for 20 minutes. The reaction mixture was quenched with saturated NH₄Cl solution (50 mL) at −20° C., and extracted with ethyl acetate (160 mL). The organic layer was separated, washed with water (200 mL) and brine (200 mL), dried over anhydrous sodium sulfate, filtered, and concentrated to give a crude product, which was purified with flash column chromatography on silica gel (ethyl acetate in petroleum ether, from 0% to 8% v/v) to give Compound 4B. LC-MS (ESI) m/z: 219 [M+H]⁺.

To a solution of Compound 4B (1.31 g, 6.01 mmol) in Et₂O (15 mL) at 0° C. was dropped neat Br₂ (961 mg, 6.01 mmol), stirred at 25° C. for 2 hours, and diluted with ethyl acetate (160 mL). The organic layer was washed with water (200 mL) and brine (200 mL), dried over anhydrous Na₂SO₄, filtered, and concentrated to give a crude Compound 4C. LC-MS (ESI) m/z: 297 [M+H]⁺.

Compounds 4D, 4E, and 4F were synthesized by employing the procedures described for Compound 2B, 2C, and 1C using 4-chloroaniline, Compounds 4C using EtOH as solvent, 4D, 4E, and 1-chloro-4-isocyanatobenzene in lieu of 4-bromoaniline, Compounds 2A using NMP as solvent, 2B, 1B, and 1-bromo-4-isocyanatobenzene.

Compound 4D. LC-MS (ESI) m/z: 344 [M+H]⁺; ¹H-NMR (CDCl₃, 400 MHz): δ (ppm) 1.48 (d, J=7.2 Hz, 3H), 4.67 (s, 1H), 5.01-5.08 (m, 1H), 6.56 (d, J=8.8 Hz, 2H), 7.13 (d, J=8.4 Hz, 2H), 7.47-7.48 (m, 1H), 7.57 (t, J=8.4 Hz, 1H), 7.84 (s, 1H), 7.93 (d, J=8.0 Hz, 1H).

Compound 4E. LC-MS (ESI) m/z: 346 [M+H]⁺; ¹H-NMR (CDCl₃, 400 MHz): δ (ppm) 1.01, 1.07 (d, J=6.4 Hz, 3H), 2.33 (d, J=4.0 Hz, 1H), 3.58-3.78 (m, 2H), 4.97 (s, 1H), 6.58-6.62 (m, 2H), 7.11-7.16 (m, 3H), 7.27-7.32 (m, 2H), 7.36-7.42 (m, 1H).

Compound 4F. LC-MS (ESI) m/z: 499 [M+H]⁺; ¹H-NMR (CDCl₃, 400 MHz): δ (ppm) 1.27 (d, J=7.2 Hz, 3H), 3.98-4.05 (m, 1H), 4.51 (s, 1H), 5.32-5.34 (m, 1H), 5.84, 5.89 (s, 1H), 7.05-7.245 (m, 7H), 7.30-7.43 (m, 5H).

To a solution of Compound 4F (300 mg, 0.60 mmol) in chloroform (5 mL) was added thionyl chloride (108 mg, 0.90 mmol). The reaction mixture was stirred at room temperature for 3 hours and concentrated under reduced pressure. The residue was purified with preparative HPLC to furnish Compound 4, which was separated with chiral HPLC to yield Compound 4-1 and Compound 4-2.

Compound 4-1: LC-MS (ESI) m/z: 481 [M+H]⁺; ¹H-NMR (CDCl₃, 400 MHz): δ (ppm) 0.86 (d, J=6.4 Hz, 3H), 4.61-4.68 (m, 1H), 5.71 (d, J=7.2 Hz, 1H), 7.08 (d, J=8.8 Hz, 2H), 7.18-7.27 (m, 5H), 7.36 (d, J=8.8 Hz, 2H), 7.46 (t, J=8.0 Hz, 1H), 7.62 (d, J=8.8 Hz, 2H). Chiral separation conditions: MeOH contained 0.2% methanol ammonia; OD-H (4.6×100 mm, 5 μm); retention time: 1.87 minutes.

Compound 4-2: LC-MS (ESI) m/z: 481 [M+H]⁺; ¹H-NMR (CDCl₃, 400 MHz): δ (ppm) 0.86 (d, J=6.4 Hz, 3H), 4.62-4.69 (m, 1H), 5.72 (d, J=7.2 Hz, 1H), 7.08 (d, J=8.8 Hz, 2H), 7.19-7.27 (m, 5H), 7.36 (d, J=8.8 Hz, 2H), 7.46 (t, J=8.0 Hz, 1H), 7.62 (d, J=8.8 Hz, 2H). Chiral separation conditions: MeOH contained 0.2% methanol ammonia; OD-H (4.6×100 mm, 5 μm); retention time: 4.29 minutes.

### Example 5

### Synthesis of 3-(1,3-bis(4-chlorophenyl)-5-methyl-2-oxoimidazolidin-4-yl) benzonitrile (5), 3-((4R,5R)-1,3-bis(4-chlorophenyl)-5-methyl-2-oxoimidazolidin-4-yl)benzonitrile (5-1), and 3-((4S,5S)-1,3-bis(4-chlorophenyl)-5-methyl-2-oxoimidazolidin-4-yl)benzonitrile (5-2)

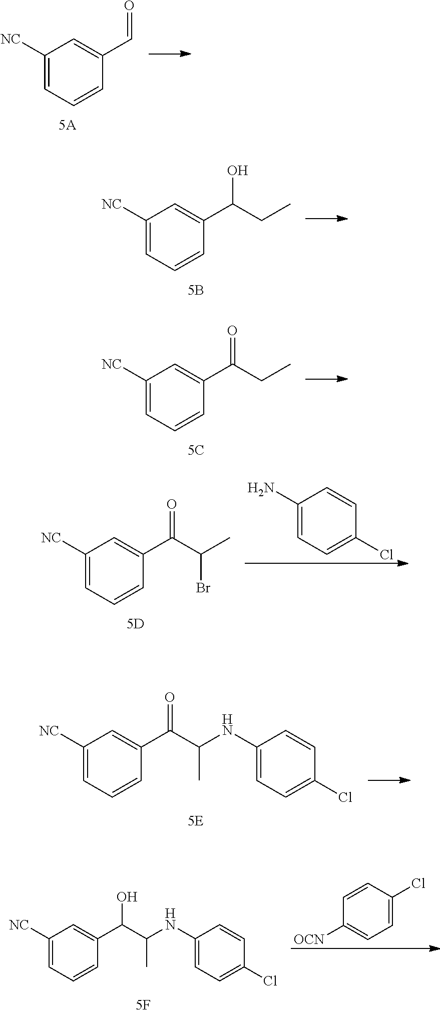

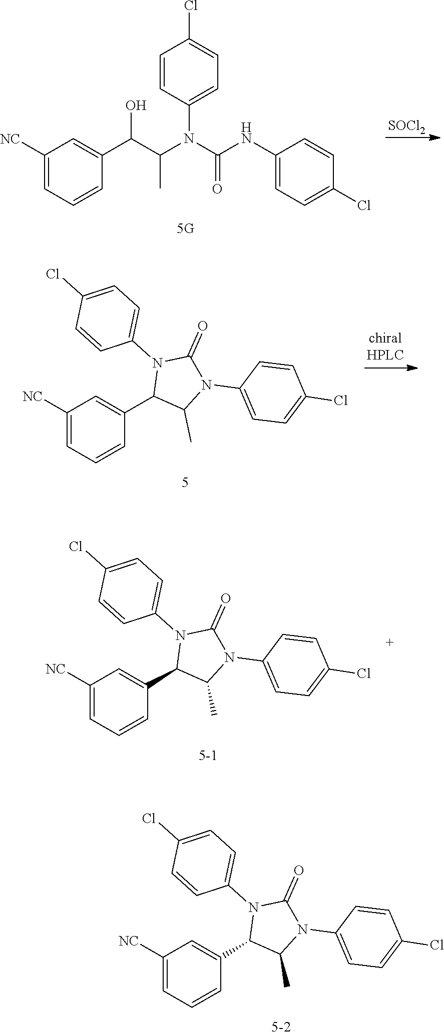 [SMILES: CC(C(O)c1cccc(C#N)c1)N(C(=O)Nc1ccc(Cl)cc1)c1ccc(Cl)cc1 ; "SOCl2" ; CC1C(c2cccc(C#N)c2)N(c2ccc(Cl)cc2)C(=O)N1c1ccc(Cl)cc1 ; C[C@@H]1[C@@H](c2cccc(C#N)c2)N(c2ccc(Cl)cc2)C(=O)N1c1ccc(Cl)cc1 ; C[C@H]1[C@H](c2cccc(C#N)c2)N(c2ccc(Cl)cc2)C(=O)N1c1ccc(Cl)cc1]

Compound 5B was synthesized by employing the procedure described for Compound 3C using EtMgBr and Compound 5A in lieu of MeMgBr and Compound 3B. LC-MS (ESI) m/z: non-ionizable compound under routine conditions used. ¹H-NMR (CDCl₃, 400 MHz): δ (ppm) 0.93 (t, J=7.6 Hz, 3H), 1.73-1.81 (m, 2H), 1.99 (d, J=3.6 Hz, 1H), 4.65-4.69 (m, 1H), 7.45 (t, J=7.6 Hz, 1H), 7.55-7.59 (m, 2H), 7.66 (s, 1H).

To a solution of Compound 5B (2.7 g, 16.8 mmol) in dichloromethane (40 mL) was added Dess-Martin periodinane (14.2 g, 33.6 mmol) at 0° C. The reaction mixture was stirred at room temperature for 4 hours, washed with saturated aqueous Na₂CO₃ solution (30 mL×3) and brine (10 mL), dried over anhydrous sodium sulfate, filtered, and concentrated under reduced pressure. The residue was purified with flash column chromatography on silica gel (ethyl acetate in petroleum ether from 0% to 15% v/v) to give Compound 5C. LC-MS (ESI) m/z: non-ionizable compound under routine conditions used; ¹H-NMR (CDCl₃, 400 MHz): δ (ppm) 1.25 (t, J=7.6 Hz, 3H), 3.02 (q, J=7.2 Hz, 2H), 7.61 (t, J=7.6 Hz, 1H), 7.82-7.85 (m, 1H), 8.18-8.24 (m, 1H), 8.24 (s, 1H).

Compounds 5D, 5E, 5F, 5G, and 5 were synthesized by employing the procedures described for Compound 4C, 2B, 2C, 1C, and 4 using Compounds 5C, 4-chloroaniline, Compounds 5D using EtOH as solvent, 5E using EtOH as solvent, 5F, 1-chloro-4-isocyanatobenzene, and 5G in lieu of Compounds 4B, 4-bromoaniline, 2A using NMP as solvent, 2B using MeOH as solvent, 1B, 1-bromo-4-isocyanatobenzene, and 4F.

Compound 5D. LC-MS (ESI) m/z: non-ionizable compound under routine conditions used; ¹H-NMR (CDCl₃, 400 MHz): δ (ppm) 1.92 (d, J=6.4 Hz, 3H), 5.21 (q, J=6.8 Hz, 1H), 7.64 (t, J=8.0 Hz, 1H), 7.86-7.88 (m, 1H), 8.24-8.26 (m, 1H), 8.31 (s, 1H).

Compound 5E. LC-MS (ESI) m/z: 285 [M+H]⁺; ¹H-NMR (CDCl₃, 400 MHz): δ (ppm) 1.48 (d, J=7.2 Hz, 3H), 4.52 (d, J=7.6 Hz, 1H), 4.98-5.06 (m, 1H), 6.54 (d, J=8.8 Hz, 2H), 7.27 (d, J=8.0 Hz, 2H), 7.67 (t, J=8.0 Hz, 1H), 7.90 (d, J=7.6 Hz, 1H), 8.22 (d, J=8.0 Hz, 1H), 8.28 (s, 1H).

Compound 5F. LC-MS (ESI) m/z: 287 [M+H]⁺.

Compound 5G. LC-MS (ESI) m/z: 440 [M+H]⁺; ¹H-NMR (CDCl₃, 400 MHz): δ (ppm) 1.24 (d, J=6.8 Hz, 3H), 3.91-3.97 (m, 1H), 4.85 (m, 1H), 5.36 (s, 1H), 5.88; 5.93 (s, 1H), 7.18-7.25 (m, 6H), 7.42-7.49 (m, 3H), 7.54-7.70 (m, 3H).

Compound 5. LC-MS (ESI) m/z: 422 [M+H]⁺; ¹H-NMR (CDCl₃, 400 MHz): δ (ppm) 0.85 (d, J=6.8 Hz, 3H), 4.64-4.70 (m, 1H), 5.74 (d, J=7.2 Hz, 1H), 7.07 (d, J=8.8 Hz, 2H), 7.23 (d, J=8.8 Hz, 2H), 7.37 (d, J=9.2 Hz, 2H), 7.56-7.69 (m, 6H). Compound 5 was separated with chiral HPLC to give Compound 5-1 and Compound 5-2.

Compound 5-1: LC-MS (ESI) m/z: 422 [M+H]⁺; ¹H-NMR (CDCl₃, 400 MHz): δ (ppm) 0.85 (d, J=6.4 Hz, 3H), 4.64-4.71 (m, 1H), 5.74 (d, J=7.2 Hz, 1H), 7.07 (d, J=9.2 Hz, 2H), 7.23 (d, J=8.8 Hz, 2H), 7.37 (d, J=8.8 Hz, 2H), 7.56-7.69 (m, 6H). Chiral separation condition: conditions: MeOH contained 0.2% Methanol Ammonia; IC (4.6×100 mm, 5 μm); retention time: 1.56 minutes.

Compound 5-2: LC-MS (ESI) m/z: 422 [M+H]⁺; ¹H-NMR (CDCl₃, 400 MHz): δ (ppm) 0.85 (d, J=6.4 Hz, 3H), 4.64-4.71 (m, 1H), 5.74 (d, J=7.6 Hz, 1H), 7.07 (d, J=8.4 Hz, 2H), 7.23 (d, J=8.8 Hz, 2H), 7.37 (d, J=9.2 Hz, 2H), 7.56-7.70 (m, 6H). Chiral separation conditions: MeOH contained 0.2% Methanol Ammonia; IC (4.6×100 mm, 5 μm); retention time: 2.43 minutes.

### Example 6

### Synthesis of 1,3-bis(4-chlorophenyl)-4,4-dimethyl-5-(3-(trifluoromethyl) phenyl)imidazolidin-2-one (6), (R)-1,3-bis(4-chlorophenyl)-4,4-dimethyl-5-(3-(trifluoromethyl)phenyl)imidazolidin-2-one (6-1), and (S)-1,3-bis(4-chlorophenyl)-4,4-dimethyl-5-(3-(trifluoromethyl)phenyl)imidazolidin-2-one (6-2)

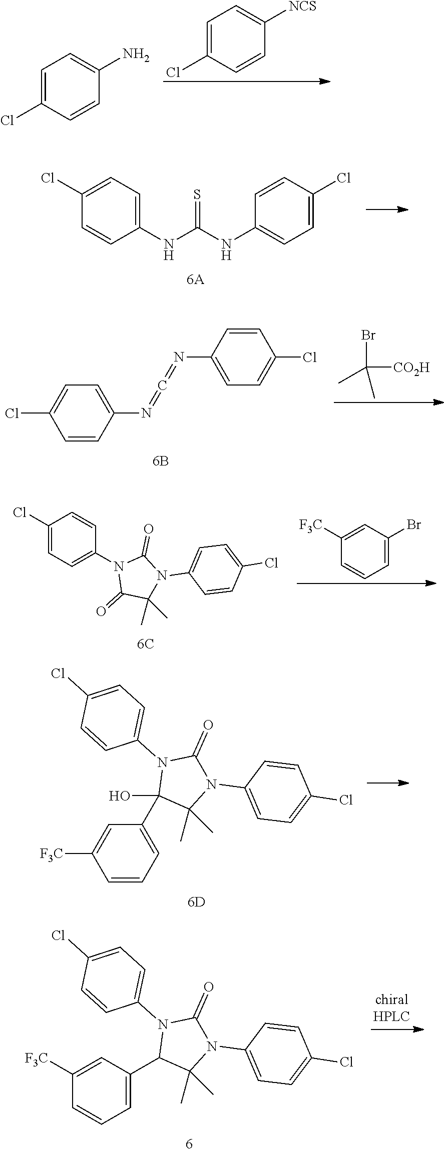

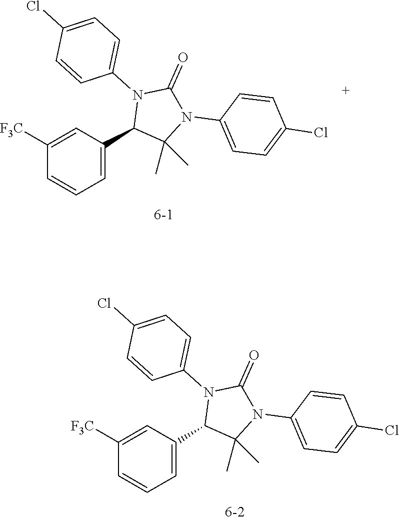 [SMILES: CC1(C)[C@@H](c2cccc(C(F)(F)F)c2)N(c2ccc(Cl)cc2)C(=O)N1c1ccc(Cl)cc1 ; CC1(C)[C@H](c2cccc(C(F)(F)F)c2)N(c2ccc(Cl)cc2)C(=O)N1c1ccc(Cl)cc1]

The solution of 4-chloroaniline (2.54 g, 20 mmol) and 1-chloro-4-isothiocynatobenzene (3.38 g, 20 mmol) in ethyl acetate (60 mL) was heated at reflux for 3 hours. The reaction mixture was concentrated under reduced pressure to furnish Compound 6A. LC-MS (ESI) m/z: 297 [M+H]⁺.

To a stirred and ice-cooled solution of Compound 6A (2.96 g, 10 mmol) in ethyl acetate (5 mL) was added triethylamine (2.02 g, 20 mmol), followed by iodine (2.79 g, 11 mmol) in several small portions over a period of 30 minutes. A light-yellow colored solid (sulfur) precipitated during the period. The precipitated sulfur was filtered, the filtrate was evaporated, and the residue was extracted with petroleum (30 mL×2). The combined extracts were concentrated under reduced pressure to give a crude Compound 6B. ¹H-NMR (400 MHz, CDCl₃): δ (ppm) 7.09 (m, 4H), 7.28 (m, 4H).

To a stirred solution of 2-bromo-2-methylpropanoic acid (588 mg, 3.52 mmol) in 1,4-dioxane (10 mL) at room temperature was added Compound 6B (1.2 g, 4.58 mmol), followed by TMP (426 mg, 3.52 mmol). After the mixture was stirred at room temperature overnight, to it was added a NaOH aqueous solution (2 N, 10 mL) and the mixture was stirred at 0° C. until the complete formation of the desired hydantoin product as showed by TLC. The resulting mixture was extracted with dichloromethane (500 mL×3). The combined extracts was extracts were dried over anhydrous Na₂SO₄, filtered, and concentrated under reduced pressure. The residue was purified with flash column chromatography on silica gel (ethyl acetate in petroleum ether, 20% v/v) to yield Compound 6C. LC-MS (ESI) m/z: 349 [M+H]⁺.

To a stirred solution of 1-bromo-3-(trifluoromethyl)benzene (321 mg, 1.43 mmol) in dry THF (5 mL) was added a solution of n-BuLi in hexane (2.5 M, 0.57 mL, 1.43 mmol) at −78° C. under nitrogen and the mixture was stirred at −78° C. for 2 minutes. The cold solution was added to a stirred solution of Compound 6C (100 mg, 0.287 mmol) in dry THF (3 mL) at −78° C. under nitrogen. The mixture was stirred at −78° C. for 1 hour, quenched with saturated NH₄Cl aqueous solution (50 mL), and extracted with ethyl acetate (30 mL×2). The combined extracts were dried over anhydrous Na₂SO₄, filtered, and concentrated under reduced pressure. The residue was purified with preparative HPLC to furnish Compound 6D. LC-MS (ESI) m/z: 495 [M+H]⁺. ¹H-NMR (400 MHz, CDCl₃): δ (ppm) 0.76 (s, 3H), 1.31 (s, 3H), 4.46 (s, 1H), 7.10-7.19 (m, 4H), 7.31-7.40 (m, 5H), 7.45 (s, 1H), 7.56 (d, J=7.6 Hz, 1H), 7.76 (s, 1H).

To a solution of Compound 6D (247 mg, 0.5 mmol) in dichloromethane (10 mL) was added triethylsilane (0.24 mL, 1.5 mmol) and boron trifluoride diethyl etherate (0.30 mL, 1.0 mmol). The resulting mixture was stirred at 10° C. for 2 hours, quenched with methanol (10 mL), and concentrated to give a crude product, which was purified with preparative HPLC to furnish Compound 6. LC-MS (ESI) m/z: 479 [M+H]⁺; ¹H-NMR (CD₃OD, 400 MHz): δ (ppm) 0.81 (s, 3H), 1.57 (s, 3H), 5.43 (s, 1H), 7.23-7.31 (m, 4H), 7.7.49 (m, 4H), 7.58-7.71 (m, 4H). Compound 6 was separated with chiral HPLC to give Compound 6-1 and Compound 6-2.

Compound 6-1: LC-MS (ESI) m/z: 479 [M+H]⁺; ¹H-NMR (CDCl₃, 400 MHz): δ (ppm) 0.78 (s, 3H), 1.52 (s, 3H), 5.02 (s, 1H), 7.17-7.20 (m, 4H), 7.35-7.47 (m, 4H), 7.53-7.61 (m, 4H). Chiral separation conditions: MeOH contained 0.2% Methanol Ammonia; OD-H (4.6×100 mm, 5 μm); retention time: 1.32 minutes.

Compound 6-2: LC-MS (ESI) m/z: 479 [M+H]⁺; ¹H-NMR (CDCl₃, 400 MHz): δ (ppm) 0.78 (s, 3H), 1.52 (s, 3H), 5.02 (s, 1H), 7.17-7.20 (m, 4H), 7.35-7.47 (m, 4H), 7.53-7.61 (m, 4H). Chiral separation conditions: MeOH contained 0.2% Methanol Ammonia; OD-H (4.6×100 mm, 5 μm); retention time: 2.08 minutes.

### Example 7

### Synthesis of 1,3-bis(4-chlorophenyl)-4,4-dimethyl-5-(3-(trifluoromethoxy) phenyl)imidazolidin-2-one (7), (R)-1,3-bis(4-chlorophenyl)-4,4-dimethyl-5-(3-(trifluoromethoxy)phenyl)imidazolidin-2-one (7-1), and (S)-1,3-bis(4-chlorophenyl)-4,4-dimethyl-5-(3-(trifluoromethoxy)phenyl)imidazolidin-2-one (7-2)

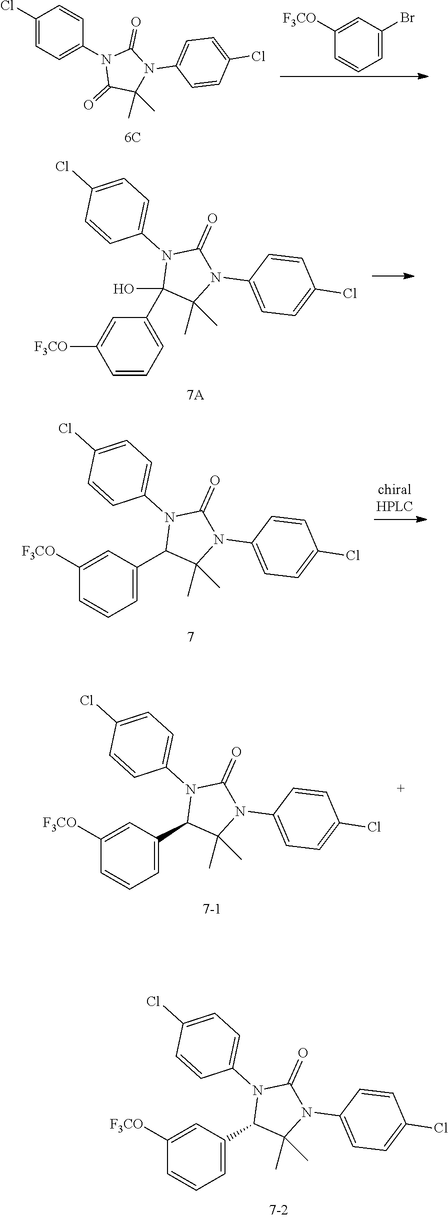 [SMILES: CC1(C)C(=O)N(c2ccc(Cl)cc2)C(=O)N1c1ccc(Cl)cc1 ; FC(F)(F)Oc1cccc(Br)c1 ; CC1(C)N(c2ccc(Cl)cc2)C(=O)N(c2ccc(Cl)cc2)C1(O)c1cccc(OC(F)(F)F)c1 ; CC1(C)C(c2cccc(OC(F)(F)F)c2)N(c2ccc(Cl)cc2)C(=O)N1c1ccc(Cl)cc1 ; CC1(C)[C@@H](c2cccc(OC(F)(F)F)c2)N(c2ccc(Cl)cc2)C(=O)N1c1ccc(Cl)cc1 ; CC1(C)[C@H](c2cccc(OC(F)(F)F)c2)N(c2ccc(Cl)cc2)C(=O)N1c1ccc(Cl)cc1]

Compounds 7A and 7 were synthesized by employing the procedures described for Compounds 6D and 6 using 1-bromo-3-(trifluoromethoxy)benzene and Compound 7A at 0° C. in lieu of 1-bromo-3-(trifluoromethyl)benzene and Compound 6D at 10° C.

Compound 7A. LC-MS (ESI) m/z: 511 [M+H]⁺. ¹H-NMR (400 MHz, CDCl₃): δ (ppm) 0.81 (s, 3H), 1.33 (s, 3H), 3.92 (s, 1H), 7.16-7.19 (m, 5H), 7.31-7.40 (m, 7H).

Compound 7. LC-MS (ESI) m/z: 495 [M+H]⁺; ¹H-NMR (CDCl₃, 400 MHz): δ (ppm) 0.79 (s, 3H), 1.52 (s, 3H), 4.97 (m, 1H), 7.17-7.20 (m, 7H), 7.36-7.41 (m, 5H). Compound 7 was separated with chiral HPLC to afford Compound 7-1 and Compound 7-2.

Compound 7-1: LC-MS (ESI) m/z: 495 [M+H]⁺; ¹H-NMR (CDCl₃, 400 MHz): δ (ppm) 0.79 (s, 3H), 1.51 (s, 3H), 4.97 (m, 1H), 7.17-7.20 (m, 7H), 7.36-7.40 (m, 5H). Chiral separation conditions: MeOH contained 0.2% Methanol Ammonia; IC (4.6×100 mm, 5 μm); retention time: 2.2 minutes.

Compound 7-2: LC-MS (ESI) m/z: 495 [M+H]⁺; ¹H-NMR (CDCl₃, 400 MHz): δ (ppm) 0.79 (s, 3H), 1.52 (s, 3H), 4.97 (m, 1H), 7.17-7.20 (m, 7H), 7.36-7.41 (m, 5H). Chiral separation conditions: MeOH contained 0.2% Methanol Ammonia; IC (4.6×100 mm, 5 μm); retention time: 3.2 minutes.

### Example 8

### Synthesis of 5-(3-chlorophenyl)-1,3-bis(4-chlorophenyl)-4,4-dimethyl imidazolidin-2-one (8), (R)-5-(3-chlorophenyl)-1,3-bis(4-chlorophenyl)-4,4-dimethyl imidazolidin-2-one (8-1), and (S)-5-(3-chlorophenyl)-1,3-bis(4-chlorophenyl)-4,4-dimethylimidazolidin-2-one (8-2)

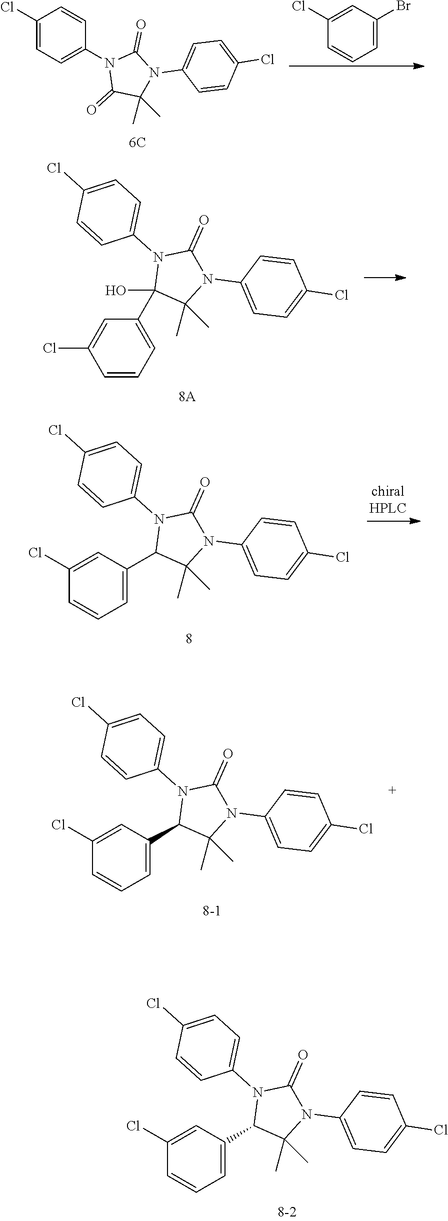 [SMILES: CC1(C)C(=O)N(c2ccc(Cl)cc2)C(=O)N1c1ccc(Cl)cc1 ; Clc1cccc(Br)c1 ; CC1(C)N(c2ccc(Cl)cc2)C(=O)N(c2ccc(Cl)cc2)C1(O)c1cccc(Cl)c1 ; CC1(C)C(c2cccc(Cl)c2)N(c2ccc(Cl)cc2)C(=O)N1c1ccc(Cl)cc1 ; CC1(C)[C@@H](c2cccc(Cl)c2)N(c2ccc(Cl)cc2)C(=O)N1c1ccc(Cl)cc1 ; CC1(C)[C@H](c2cccc(Cl)c2)N(c2ccc(Cl)cc2)C(=O)N1c1ccc(Cl)cc1]

Compounds 8A and 8 were synthesized by employing the procedures described for Compounds 6D and 6 using 1-bromo-3-chlorobenzene and Compound 8A in lieu of 1-bromo-3-(trifluoromethyl)benzene and Compound 6D.

Compound 8A. LC-MS (ESI) m/z: 461 [M+H]⁺. ¹H-NMR (400 MHz, Acetone-d₆): δ (ppm) 0.90 (s, 3H), 1.44 (s, 3H), 6.57 (s, 1H), 7.23 (d, J=8.8 Hz, 2H), 7.34-7.36 (m, 2H), 7.42-7.44 (m, 3H), 7.49 (d, J=8.8 Hz, 2H), 7.57-7.59 (m, 3H).

Compound 8. LC-MS (ESI) m/z: 445 [M+H]⁺; ¹H-NMR (CD₃OD, 400 MHz): δ (ppm) 0.83 (s, 3H), 1.55 (s, 3H), 5.30 (s, 1H), 7.24-7.31 (m, 5H), 7.34-7.40 (m, 3H), 7.44-7.49 (m, 4H). Compound 8 was separated with chiral HPLC to give Compound 8-1 and Compound 8-2.

Compound 8-1: LC-MS (ESI) m/z: 445 [M+H]⁺; ¹H-NMR (CDCl₃, 400 MHz): δ (ppm) 0.81 (s, 3H), 1.50 (s, 3H), 4.91 (s, 1H), 7.15-7.20 (m, 5H), 7.27-7.30 (m, 3H), 7.36-7.41 (m, 4H). Chiral separation conditions: MeOH contained 0.2% Methanol Ammonia; OD-H (4.6×100 mm, 5 μm); retention time: 1.33 minutes.

Compound 8-2: LC-MS (ESI) m/z: 445 [M+H]⁺; ¹H-NMR (CDCl₃, 400 MHz): δ (ppm) 0.81 (s, 3H), 1.50 (s, 3H), 4.91 (s, 1H), 7.15-7.20 (m, 5H), 7.27-7.31 (m, 3H), 7.37-7.41 (m, 4H). Chiral separation conditions: MeOH contained 0.2% Methanol Ammonia; OD-H (4.6×100 mm, 5 μm); retention time: 2.07 minutes.

### Example 9

### Synthesis of 1,3-bis(4-chlorophenyl)-5-(3-(difluoromethoxy)phenyl)-4,4-dimethylimidazolidin-2-one (9), (R)-1,3-bis(4-chlorophenyl)-5-(3-(difluoromethoxy)phenyl)-4,4-dimethylimidazolidin-2-one (9-1), and (S)-1,3-bis(4-chlorophenyl)-5-(3-(difluoromethoxy)phenyl)-4,4-dimethylimidazolidin-2-one (9-2)

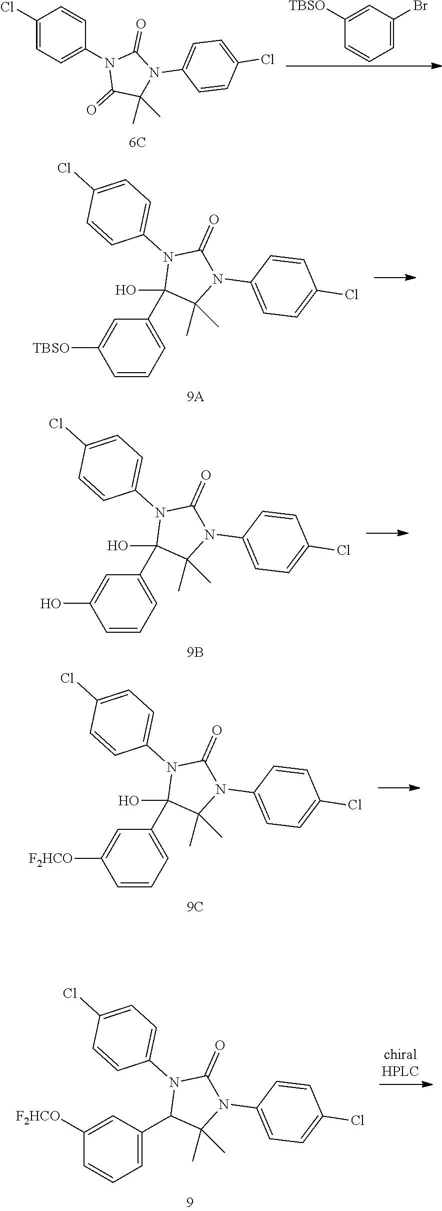 [SMILES: CC1(C)C(=O)N(c2ccc(Cl)cc2)C(=O)N1c1ccc(Cl)cc1 ; CC(C)(C)[Si](C)(C)Oc1cccc(Br)c1 ; CC1(C)N(c2ccc(Cl)cc2)C(=O)N(c2ccc(Cl)cc2)C1(O)c1cccc(O[Si](C)(C)C(C)(C)C)c1 ; CC1(C)N(c2ccc(Cl)cc2)C(=O)N(c2ccc(Cl)cc2)C1(O)c1cccc(O)c1 ; CC1(C)N(c2ccc(Cl)cc2)C(=O)N(c2ccc(Cl)cc2)C1(O)c1cccc(OC(F)F)c1 ; CC1(C)C(c2cccc(OC(F)F)c2)N(c2ccc(Cl)cc2)C(=O)N1c1ccc(Cl)cc1]

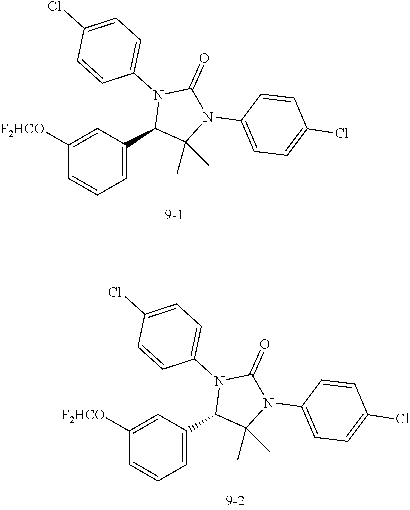 [SMILES: CC1(C)[C@@H](c2cccc(OC(F)F)c2)N(c2ccc(Cl)cc2)C(=O)N1c1ccc(Cl)cc1 ; CC1(C)[C@H](c2cccc(OC(F)F)c2)N(c2ccc(Cl)cc2)C(=O)N1c1ccc(Cl)cc1]

Compound 9A was synthesized by employing the procedure described for Compound 6D using (3-bromophenoxy)(tert-butyl)dimethylsilane in lieu of 1-bromo-3-(trifluoromethyl)benzene. LC-MS (ESI) m/z: 557 [M+H]⁺.

A mixture of Compound 9A (300 mg, 0.4 mmol) and TBAF (183 mg, 0.69 mmol) in THF (5 mL) was stirred at room temperature for 2 hours. The reaction mixture was diluted with water (50 mL) and extracted with ethyl acetate (20 mL×3). The combined extracts were dried over anhydrous Na₂SO₄, filtered, and concentrated under reduced pressure. The residue was purified by preparative HPLC to afford Compound 9B. LC-MS (ESI) m/z: 443 [M+H]⁺. ¹H-NMR (400 MHz, CD₃OD): δ (ppm) 0.85 (s, 3H), 1.34 (s, 3H), 6.71 (dd, J=1.6, 7.6 Hz, 1H), 6.90 (d, J=7.2 Hz, 1H), 6.96 (s, 1H), 7.10 (t, J=8.0 Hz, 1H), 7.21 (d, J=8.4 Hz, 2H), 7.37 (d, J=8.4 Hz, 2H), 7.48 (d, J=8.4 Hz, 2H), 7.54 (d, J=8.4 Hz, 2H).

The mixture of Compound 9B (100 mg, 0.226 mmol), Cs₂CO₃ (147 mg, 0.452 mmol), and ClCF₂COONa (86 mg, 0.565 mmol) in DMF (5 mL) was heated at 100° C. overnight. The reaction mixture was diluted with water (50 mL) and extracted with ethyl acetate (20 mL×3). The combined extracts were dried over anhydrous Na₂SO₄, filtered, and concentrated under reduced pressure. The residue was purified with preparative HPLC to furnish Compound 9C. LC-MS (ESI) m/z: 493 [M+H]⁺. ¹H-NMR (400 MHz, CDCl₃): δ (ppm) 0.82 (s, 3H), 1.34 (s, 3H), 3.79 (s, 1H), 6.43 (t, J=73 Hz, 1H), 7.07 (d, J=8.0 Hz, 1H), 7.16-7.22 (m, 6H), 7.27-7.31 (m, 1H), 7.36-7.41 (m, 4H).

Compound 9 was synthesized by employing the procedure described for Compound 6 using Compound 9C at room temperature in lieu of Compound 6D at 10° C. LC-MS (ESI) m/z: 477 [M+H]⁺; ¹H-NMR (400 MHz, CDCl₃): δ (ppm) 0.80 (s, 3H), 1.51 (s, 3H), 4.94 (s, 1H), 6.48 (t, J=73 Hz, 1H), 7.04-7.14 (m, 3H), 7.17-7.20 (m, 4H), 7.34-7.40 (m, 5H). Compound 9 was separated with chiral HPLC to give Compound 9-1 and Compound 9-2.

Compound 9-1: LC-MS (ESI) m/z: 477 [M+H]⁺; ¹H-NMR (400 MHz, CDCl₃): δ (ppm) 0.80 (s, 3H), 1.51 (s, 3H), 4.94 (s, 1H), 6.48 (t, J=73 Hz, 1H), 7.04-7.13 (m, 3H), 7.17-7.20 (m, 4H), 7.34-7.40 (m, 5H). Chiral separation conditions: MeOH contained 0.2% Methanol Ammonia; OD-H (4.6×100 mm, 5 μm); retention time: 1.69 minutes.

Compound 9-2: LC-MS (ESI) m/z: 477 [M+H]⁺; ¹H-NMR (400 MHz, CDCl₃): δ (ppm) 0.80 (s, 3H), 1.51 (s, 3H), 4.94 (s, 1H), 6.48 (t, J=73 Hz, 1H), 7.04-7.14 (m, 3H), 7.17-7.20 (m, 4H), 7.34-7.40 (m, 5H). Chiral separation conditions: MeOH contained 0.2% Methanol Ammonia; OD-H (4.6×100 mm, 5 μm); retention time: 2.77 minutes.

### Example 10

### Synthesis of 3-(1,3-bis(4-chlorophenyl)-5,5-dimethyl-2-oxoimidazolidin-4-yl)benzonitrile (10), (R)-3-(1,3-bis(4-chlorophenyl)-5,5-dimethyl-2-oxoimidazolidin-4-yl)benzonitrile (10-1), and (S)-3-(1,3-bis(4-chlorophenyl)-5,5-dimethyl-2-oxoimidazolidin-4-yl)benzonitrile (10-2)

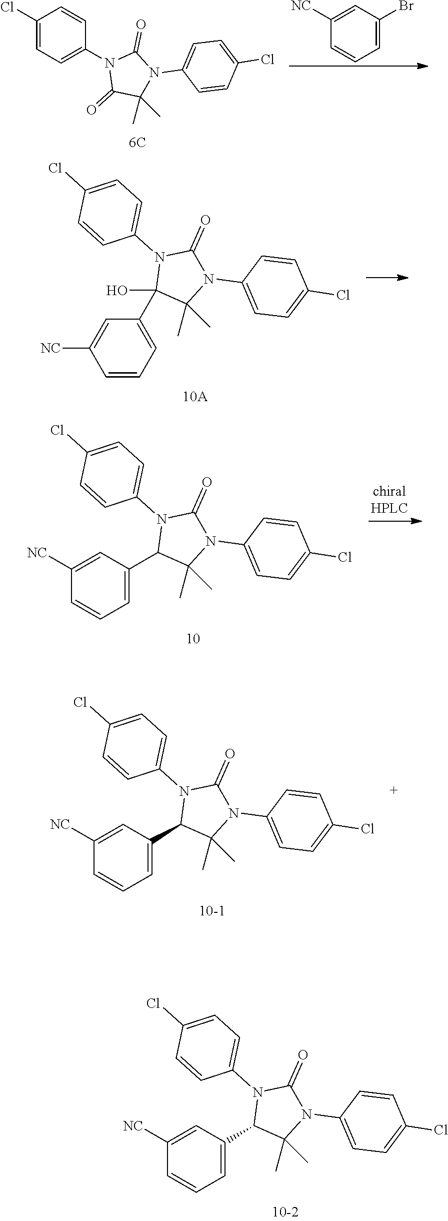 [SMILES: CC1(C)C(=O)N(c2ccc(Cl)cc2)C(=O)N1c1ccc(Cl)cc1 ; N#Cc1cccc(Br)c1 ; CC1(C)N(c2ccc(Cl)cc2)C(=O)N(c2ccc(Cl)cc2)C1(O)c1cccc(C#N)c1 ; CC1(C)C(c2cccc(C#N)c2)N(c2ccc(Cl)cc2)C(=O)N1c1ccc(Cl)cc1 ; CC1(C)[C@@H](c2cccc(C#N)c2)N(c2ccc(Cl)cc2)C(=O)N1c1ccc(Cl)cc1 ; CC1(C)[C@H](c2cccc(C#N)c2)N(c2ccc(Cl)cc2)C(=O)N1c1ccc(Cl)cc1]

Compounds 10A and 10 were synthesized by employing the procedures described for Compounds 6D and 6 using 3-bromobenzonitrile and Compound 10A in lieu of 1-bromo-3-(trifluoromethyl)benzene and Compound 6D.

Compound 10A. LC-MS (ESI) m/z: 452 [M+H]⁺. ¹H-NMR (400 MHz, CDCl₃): δ (ppm) 0.77 (s, 3H), 1.30 (s, 3H), 4.48 (s, 1H), 7.11-7.19 (m, 4H), 7.32-7.40 (m, 5H), 7.52 (s, 1H), 7.59 (d, J=7.6 Hz, 1H), 7.78 (s, 1H).

Compound 10. LC-MS (ESI) m/z: 436 [M+H]⁺; ¹H-NMR (CDCl₃, 400 MHz): δ (ppm) 0.79 (s, 3H), 1.52 (s, 3H), 5.00 (s, 1H), 7.17-7.21 (m, 4H), 7.34 (d, J=8.4 Hz, 2H), 7.40 (d, J=8.4 Hz, 2H), 7.47-7.53 (m, 2H), 7.58-7.65 (m, 2H). Compound 10 was separated with chiral HPLC to give Compound 10-1 and Compound 10-2.

Compound 10-1: LC-MS (ESI) m/z: 436 [M+H]⁺; ¹H-NMR (CDCl₃, 400 MHz): δ (ppm) 0.79 (s, 3H), 1.52 (s, 3H), 5.00 (s, 1H), 7.17-7.20 (m, 4H), 7.34 (d, J=8.8 Hz, 2H), 7.40 (d, J=8.8 Hz, 2H), 7.47-7.65 (m, 4H). Chiral separation conditions: MeOH contained 0.2% Methanol Ammonia; OD-H (4.6×100 mm, 5 μm); retention time: 1.11 minutes.

Compound 10-2: LC-MS (ESI) m/z: 436 [M+H]⁺; ¹H-NMR (CDCl₃, 400 MHz): δ (ppm) 0.79 (s, 3H), 1.52 (s, 3H), 5.00 (s, 1H), 7.17-7.21 (m, 4H), 7.34 (d, J=8.8 Hz, 2H), 7.40 (d, J=8.8 Hz, 2H), 7.47-7.65 (m, 4H). Chiral separation conditions: MeOH contained 0.2% Methanol Ammonia; OD-H (4.6×100 mm, 5 μm); retention time: 1.53 minutes.

### Example 11

### Synthesis of 1,3-bis(4-bromophenyl)-4-methyl-5-(3-(trifluoromethoxy) phenyl)imidazolidin-2-one (11), (4R,5R)-1,3-bis(4-bromophenyl)-4-methyl-5-(3-(trifluoromethoxy)phenyl)imidazolidin-2-one (11-1), and (4S,5S)-1,3-bis(4-bromophenyl)-4-methyl-5-(3-(trifluoromethoxy)phenyl)imidazolidin-2-one (11-2)

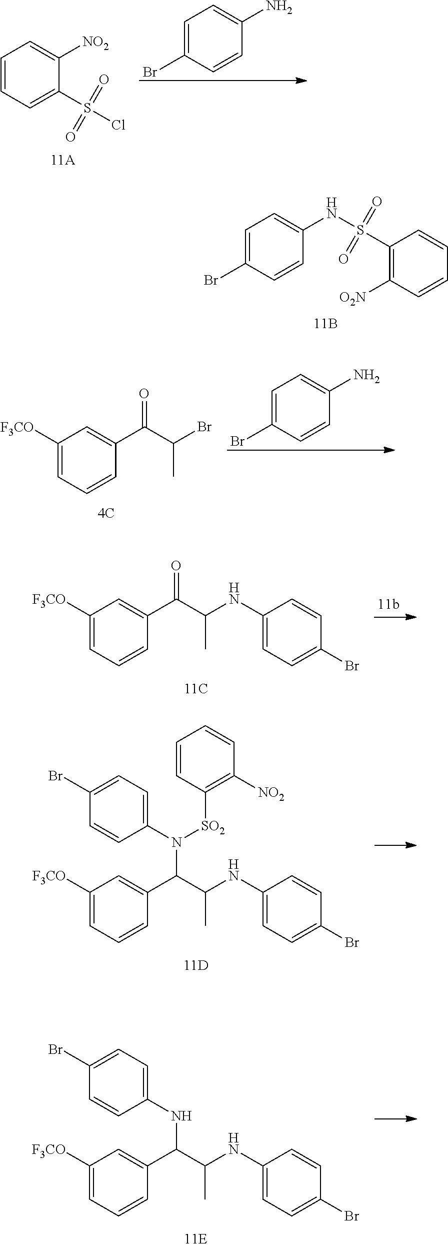

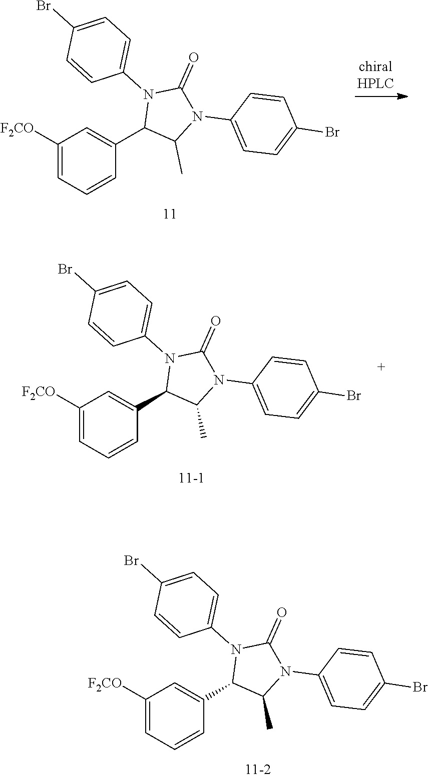

To a solution of 4-bromoaniline (1.0 g, 5.8 mmol) in dichloromethane (20 mL) was added triethylamine (1.75 g, 17.4 mmol), followed by 2-nitrobenzene-1-sulfonyl chloride 11A (1.3 g, 5.8 mmol). The mixture was stirred at room temperature for 4 hours and washed with water (15 mL). The organic layer was dried over anhydrous sodium sulfate, filtered, and concentrated under reduced pressure. The residue was purified with flash column chromatography on silica gel (ethyl acetate in petroleum ether from 0% to 40%) to yield Compound 11B. LC-MS (ESI) m/z: 357 [M+H]⁺.

Compound 11C was synthesized by employing the procedure described for Compound 2B using Compound 4C using Et₃N as base THF as solvent at 75° C., in lieu of Compound 2A using NaHCO₃ as base NMP as solvent at 60° C. LC-MS (ESI) m/z: 388 [M+H]⁺; ¹H-NMR (CDCl₃, 400 MHz): δ (ppm) 1.48 (d, J=6.4 Hz, 3H), 4.67 (s, 1H), 5.02 (s, 1H), 6.54 (d, J=8.8 Hz, 2H), 7.25-7.28 (m, 2H), 7.47-7.49 (m, 1H), 7.57 (t, J=8.0 Hz, 1H), 7.84 (s, 1H), 7.93 (d, J=8.0 Hz, 1H).

To a solution of Compound 11C (218 mg, 0.56 mmol) in anhydrous toluene (10 mL) was added Compound 11B (200 mg, 0.56 mmol), PPh₃ (293 mg, 0.12 mmol), and DIAD (226 mg, 1.12 mmol) at room temperature and heated at 105° C. under nitrogen overnight. The mixture was concentrated and purified with flash column chromatography on silica gel (ethyl acetate in petroleum ether from 0% to 40%) to yield Compound 11D. LC-MS (ESI) m/z: 728 [M+H]⁺.

To a solution of Compound 11D (200 mg, 0.275 mmol) in anhydrous DMF (8 mL) was added potassium carbonate (114 mg, 0.825 mmol) and benzenethiol (36 mg, 0.33 mmol) at room temperature and the mixture was stirred at room temperature for 3 hours. The reaction mixture was diluted with water (20 mL) and extracted with ethyl acetate (20 mL×3). The combined extracts were dried over anhydrous sodium sulfate, filtered, and concentrated to give a crude product, which was purified with flash column chromatography on silica gel (ethyl acetate in petroleum ether from 0% to 40%) to yield Compound 11E. LC-MS (ESI) m/z: 543 [M+H]⁺; ¹H-NMR (CDCl₃, 400 MHz): δ (ppm) 1.09 (d, J=6.4 Hz, 3H), 3.37 (d, J=10 Hz, 1H), 3.96-4.01 (m, 1H), 4.55-4.64 (m, 2H), 6.36 (d, J=9.2 Hz, 2H), 6.57 (d, J=8.8 Hz, 2H), 7.14-7.17 (m, 4H), 7.14-7.25 (m, 1H), 7.27-7.30 (m, 2H), 7.38-7.42 (m, 1H).

Compound 11 was synthesized by employing the procedure described for Compound 2 using Compound 11E in lieu of Compound 2E. Compound 11 was separated with chiral HPLC to give Compound 11-1 and Compound 11-2.

Compound 11-1: LC-MS (ESI) m/z: 569 [M+H]⁺; ¹H-NMR (CDCl₃, 400 MHz): δ (ppm) 0.81 (d, J=6.4 Hz, 3H), 4.65-4.72 (m, 1H), 5.39 (d, J=8.8 Hz, 1H), 7.11-7.20 (m, 3H), 7.30-7.41 (m, 7H), 7.51 (d, J=8.8 Hz, 2H). Chiral separation conditions: MeOH contained 0.2% Methanol Ammonia; OD-H (4.6×100 mm, 5 μm); retention time: 2.65 minutes.

Compound 11-2: LC-MS (ESI) m/z: 569 [M+H]⁺; ¹H-NMR (CDCl₃, 400 MHz): δ (ppm) 0.81 (d, J=6.4 Hz, 3H), 4.65-4.72 (m, 1H), 5.39 (d, J=8.8 Hz, 1H), 7.11-7.20 (m, 3H), 7.30-7.41 (m, 7H), 7.51 (d, J=8.8 Hz, 2H). Chiral separation conditions: MeOH contained 0.2% Methanol Ammonia; OD-H (4.6×100 mm, 5 μm); retention time: 3.16 minutes.

### Example 12

### Synthesis of 1,3-bis(4-chlorophenyl)-3a-(3-(trifluoromethoxy)phenyl) hexahydrocyclopenta[d]imidazol-2(1H)-one (12), (3aS,6aS)-1,3-bis(4-chlorophenyl)-3a-(3-(trifluoromethoxy)phenyl) hexahydrocyclopenta[d]imidazol-2(1H)-one (12-1), and (3 aR,6aR)-1,3-bis(4-chlorophenyl)-3a-(3-(trifluoromethoxy)phenyl) hexahydrocyclopenta[d]imidazol-2(1H)-one (12-2)

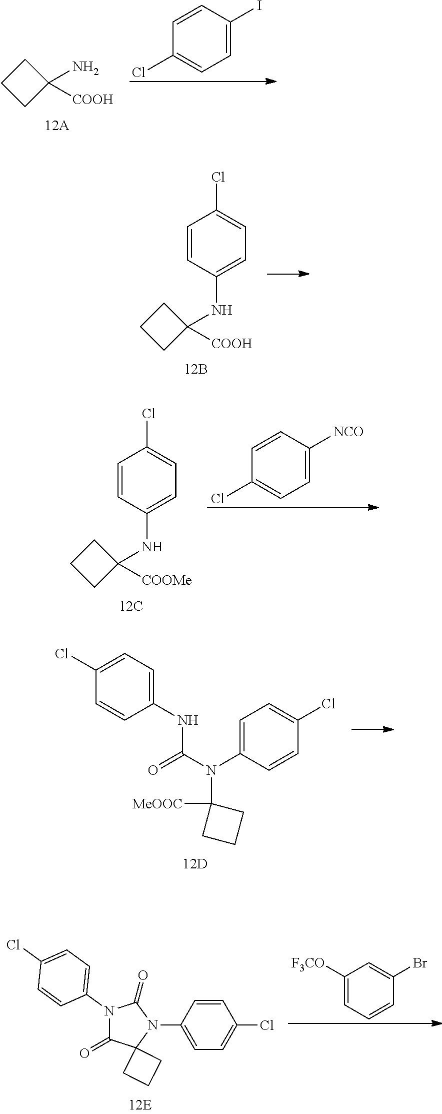

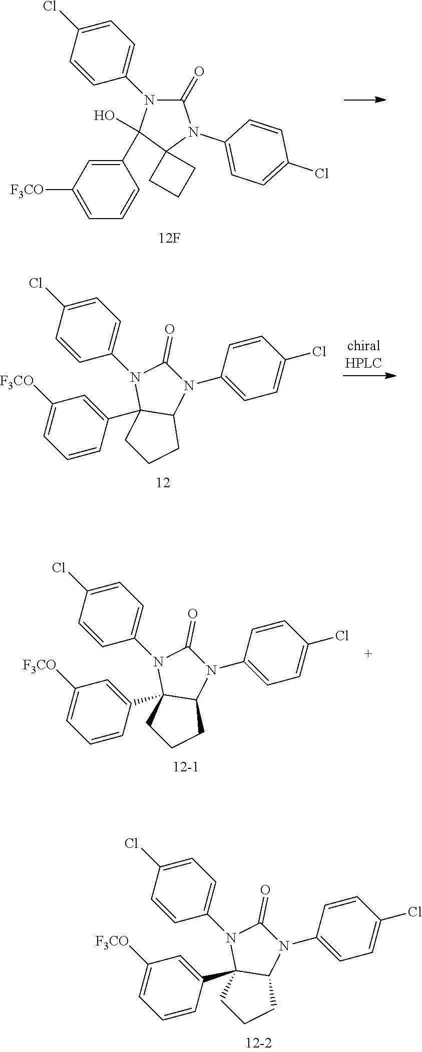 [SMILES: O=C1N(c2ccc(Cl)cc2)C2(CCC2)C(O)(c2cccc(OC(F)(F)F)c2)N1c1ccc(Cl)cc1 ; O=C1N(c2ccc(Cl)cc2)C2CCCC2(c2cccc(OC(F)(F)F)c2)N1c1ccc(Cl)cc1 ; O=C1N(c2ccc(Cl)cc2)[C@H]2CCC[C@@]2(c2cccc(OC(F)(F)F)c2)N1c1ccc(Cl)cc1 ; O=C1N(c2ccc(Cl)cc2)[C@@H]2CCC[C@]2(c2cccc(OC(F)(F)F)c2)N1c1ccc(Cl)cc1]

To a solution of Compound 12A (2 g, 15.5 mmol) and 1-chloro-4-iodobenzene (5.53 g, 23.2 mmol) in DMF/H₂O (24 mL, 5/1 in volume) was added K₂CO₃ (6.4 g, 46.5 mmol), Et₃N (156 mg, 1.55 mmol), 2-acetylcyclohexanone (2.17 g, 15.5 mmol), and CuI (582 mg, 3.1 mmol) and heated at 110° C. overnight. The reaction mixture was diluted with H₂O (100 mL), its pH adjusted to about 4 with diluted HCl solution (1 N), and extracted with EtOAc (50 mL×2). The combined extracts were washed with brine (50 mL), dried over anhydrous sodium sulfate, filtered, and concentrated to give a crude Compound 12B, which was used directly in the next step without further purification. LC-MS (ESI) m/z: 226 [M+H]⁺.

To a solution of Compound 12B (2.1 g, 9 mmol) in methanol (30 mL) was dropped concentrated sulfuric acid (1.5 mL) at room temperature. The mixture was heated at 60° C. overnight and concentrated under reduced pressure. The residue was dissolved in ethyl acetate (30 mL) and washed with water (20 mL), 0.5 M aqueous sodium bicarbonate solution (20 mL), and brine (20 mL), successively. The organic layer was dried over anhydrous sodium sulfate, filtered, and concentrated to give a crude product, which was purified with chromatography using eluent (ethyl acetate in petroleum ether, 10% v/v) to furnish Compound 12C. LC-MS (ESI) m/z: 240 [M+H]. ¹H-NMR (400 MHz, CDCl₃): δ (ppm) 2.02-2.13 (m, 2H), 2.17-2.19 (m, 2H), 2.68-2.74 (m, 2H), 3.68 (s, 3H), 4.29 (s, 1H), 7.35-7.38 (m, 2H), 7.07-7.10 (m, 2H).

A mixture of Compound 12C (2 g, 8.36 mmol) and 1-chloro-4-isocyanatobenzene (2.5 g, 16.72 mmol) in toluene (20 mL) was heated at 80° C. overnight. The reaction mixture was concentrated under reduced pressure. The residue was purified with chromatography using eluent (ethyl acetate in petroleum ether, 20% v/v) to furnish Compound 12D. LC-MS (ESI) m/z: 393 [M+H]⁺.

To a solution of Compound 12D (1.6 g, 0.61 mmol) in MeOH (15 mL) was added 5 drops of 25% NH₃ solution in methanol. The reaction was stirred at room temperature for 1.5 hours, with completion monitored by LC-MS. The mixture was evaporated to dryness. The resulting crystalline product was recrystallized from EtOAc/petroleum ether (1:1 in volume) to furnish Compound 12E. LC-MS (ESI) m/z: 361 [M+H]⁺. ¹H-NMR (400 MHz, CDCl₃): δ (ppm) 1.57-1.69 (m, 1H), 2.20-2.23 (m, 1H), 2.41-2.49 (m, 2H), 2.58-2.65 (m, 2H), 7.26-7.30 (m, 2H), 7.43-7.52 (m, 6H).

Compounds 12F and 12 were synthesized by employing the procedures described for Compounds 6D and 6 using 1-bromo-3-(trifluoromethoxy)benzene, Compounds 12E, and 12F at room temperature in lieu of 1-bromo-3-(trifluoromethyl)benzene, Compounds 6C, and 6D at 10° C.

Compound 12F. LC-MS (ESI) m/z: 523 [M+H]⁺. ¹H-NMR (400 MHz, CDCl₃): δ (ppm) 0.83-0.94 (m, 1H), 1.08-1.16 (m, 1H), 1.74-1.80 (m, 1H), 1.98-2.06 (m, 1H), 2.22-2.30 (m, 1H), 2.69-2.75 (m, 1H), 3.66 (s, 1H), 7.14-7.26 (m, 5H), 7.33-7.44 (m, 7H).

Compound 12. LC-MS (ESI) m/z: 507 [M+H]⁺; ¹H-NMR (CDCl₃, 400 MHz): δ (ppm) 1.93-2.05 (m, 3H), 2.13-2.19 (m, 1H), 2.28-2.39 (m, 2H), 4.39-4.42 (m, 1H), 6.95-6.99 (m, 2H), 7.19-7.23 (m, 4H), 7.30-7.45 (m, 4H), 7.57-7.60 (m, 2H). Compound 12 was separated with chiral HPLC to furnish Compound 12-1 and Compound 12-2.

Compound 12-1: LC-MS (ESI) m/z: 507 [M+H]⁺; ¹H-NMR (CDCl₃, 400 MHz): δ (ppm) 1.91-2.05 (m, 3H), 2.12-2.21 (m, 1H), 2.27-2.41 (m, 2H), 4.39-4.42 (m, 1H), 6.95-6.99 (m, 2H), 7.19-7.23 (m, 4H), 7.30-7.45 (m, 4H), 7.57-7.60 (m, 2H). Chiral separation conditions: MeOH contained 0.2% methanol ammonia; OJ (4.6×250 mm, 5 μm); retention time: 2.9 minutes.

Compound 12-2: LC-MS (ESI) m/z: 507 [M+H]⁺; ¹H-NMR (CDCl₃, 400 MHz): δ (ppm) 1.93-2.05 (m, 3H), 2.13-2.17 (m, 1H), 2.28-2.41 (m, 2H), 4.39-4.42 (m, 1H), 6.95-6.99 (m, 2H), 7.18-7.23 (m, 4H), 7.30-7.45 (m, 4H), 7.57-7.60 (m, 2H). Chiral separation conditions: MeOH contained 0.2% methanol ammonia; OJ (4.6×250 mm, 5 μm); retention time: 4.0 min.

### Example 13

### Synthesis of 1,3-bis(4-chlorophenyl)-3a-(3-(trifluoromethyl)phenyl) hexahydrocyclopenta[d]imidazol-2(1H)-one (13), (3aS,6aS)-1,3-bis(4-chlorophenyl)-3a-(3-(trifluoromethyl)phenyl)hexahydrocyclopenta[d]imidazol-2(1H)-one (13-1), and (3aR,6aR)-1,3-bis(4-chlorophenyl)-3a-(3-(trifluoromethyl)phenyl)hexahydro cyclopenta[d]imidazol-2(1H)-one (13-2)

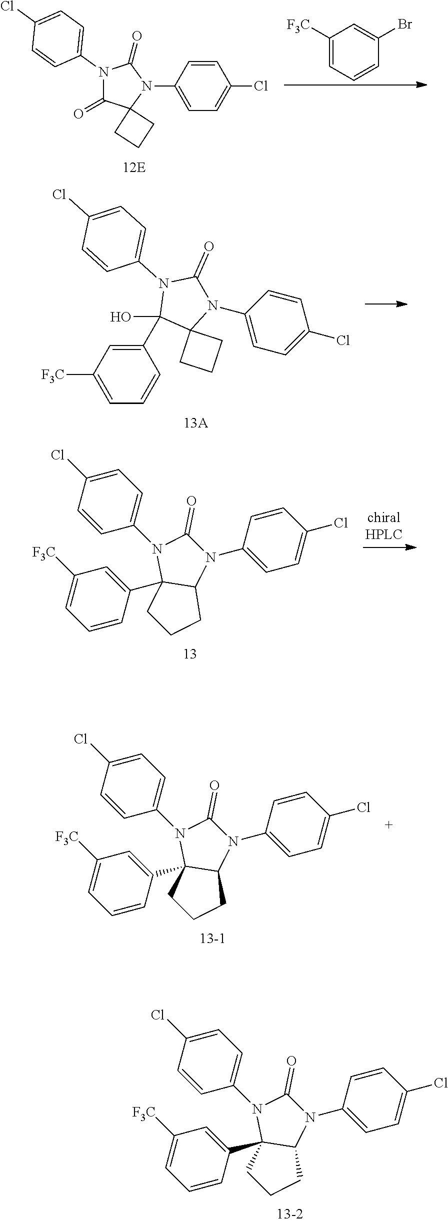 [SMILES: O=C1N(c2ccc(Cl)cc2)C(=O)C2(CCC2)N1c1ccc(Cl)cc1 ; FC(F)(F)c1cccc(Br)c1 ; O=C1N(c2ccc(Cl)cc2)C2(CCC2)C(O)(c2cccc(C(F)(F)F)c2)N1c1ccc(Cl)cc1 ; O=C1N(c2ccc(Cl)cc2)C2CCCC2(c2cccc(C(F)(F)F)c2)N1c1ccc(Cl)cc1 ; O=C1N(c2ccc(Cl)cc2)[C@H]2CCC[C@@]2(c2cccc(C(F)(F)F)c2)N1c1ccc(Cl)cc1 ; O=C1N(c2ccc(Cl)cc2)[C@@H]2CCC[C@]2(c2cccc(C(F)(F)F)c2)N1c1ccc(Cl)cc1]

Compounds 13A and 13 were synthesized by employing the procedures described for Compounds 6D and 6 using Compounds 12E and 13A at room temperature in lieu of Compounds 6C and 6D at 10° C.

Compound 13A. LC-MS (ESI) m/z: 507 [M+H]⁺. ¹H-NMR (400 MHz, CDCl₃): δ (ppm) 0.76-0.87 (m, 1H), 1.03-1.07 (m, 1H), 1.62-1.69 (m, 1H), 1.98-2.03 (m, 1H), 2.16-2.24 (m, 1H), 2.66-2.73 (m, 1H), 4.38 (s, 1H), 7.13-7.17 (m, 4H), 7.26-7.29 (m, 2H), 7.36-7.38 (m, 2H), 7.39-7.43 (m, 1H), 7.57-7.59 (m, 2H), 7.85 (s, 1H).

Compound 13 was separated with chiral HPLC to afford Compound 13-1, and Compound 13-2.

Compound 13-1: LC-MS (ESI) m/z: 491 [M+H]⁺; ¹H-NMR (CDCl₃, 400 MHz): δ (ppm) 1.94-2.06 (m, 4H), 2.31-2.44 (m, 2H), 4.37-4.39 (m, 1H), 6.96 (d, J=8.8 Hz, 2H), 7.20 (d, J=8.8 Hz, 2H), 7.31 (d, J=8.8 Hz, 2H), 7.50-7.63 (m, 6H). Chiral separation conditions: MeOH contained 0.2% methanol ammonia; S,S-WHELK-Ol (4.6×100 mm, 5 um); retention time: 3.49 minutes.

Compound 13-2: LC-MS (ESI) m/z: 491 [M+H]⁺; ¹H-NMR (CDCl₃, 400 MHz): δ (ppm) 1.94-2.16 (m, 4H), 2.31-2.46 (m, 2H), 4.37-4.39 (m, 1H), 6.96 (d, J=8.8 Hz, 2H), 7.20 (d, J=8.8 Hz, 2H), 7.31 (d, J=8.8 Hz, 2H), 7.50-7.63 (m, 6H). Chiral separation conditions: MeOH contained 0.2% methanol ammonia; S,S-WHELK-Ol (4.6×100 mm, 5 um); retention time: 2.31 minutes.

### Example 14

### Synthesis of 3-(1,3-bis(4-chlorophenyl)-2-oxooctahydro-3aH-benzo[d]imidazol-3a-yl)benzonitrile (14), 3-((3 aR,7aR)-1,3-bis(4-chlorophenyl)-2-oxooctahydro-3aH-benzo[d]imidazol-3a-yl)benzonitrile (14-1), and 3-((3 aS,7aS)-1,3-bis(4-chlorophenyl)-2-oxooctahydro-3 aH-benzo[d]imidazol-3a-yl)benzonitrile (14-2)

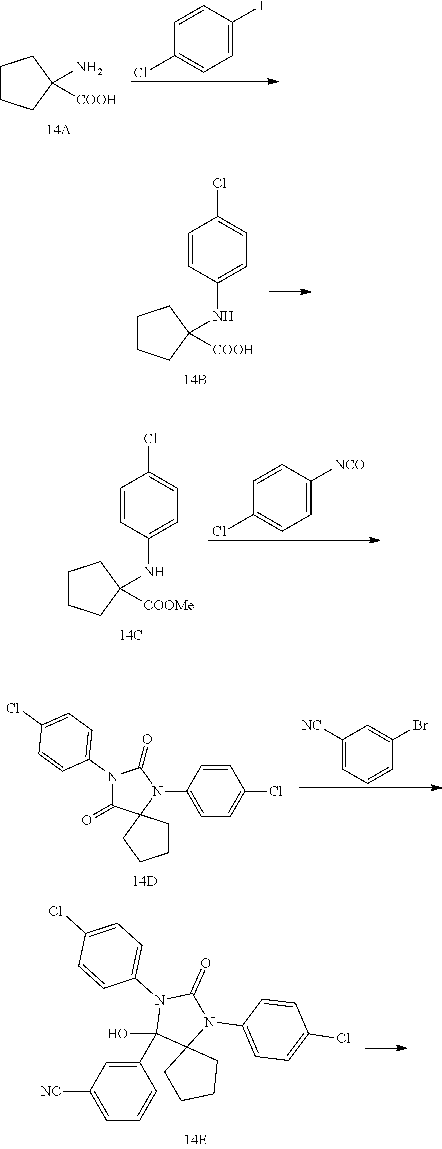

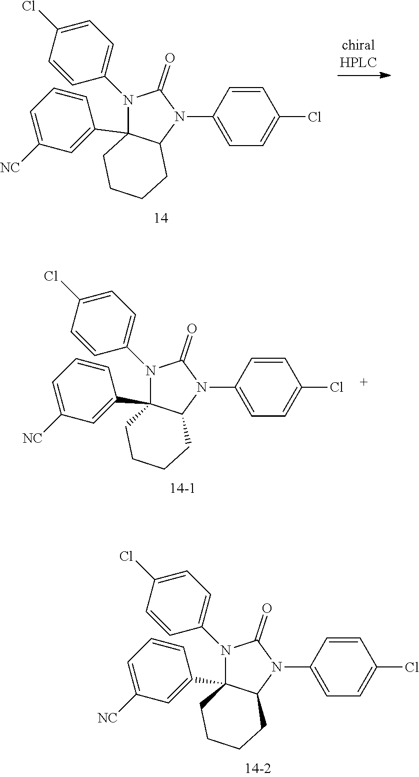 [SMILES: N#Cc1cccc(C23CCCCC2N(c2ccc(Cl)cc2)C(=O)N3c2ccc(Cl)cc2)c1 ; N#Cc1cccc([C@]23CCCC[C@H]2N(c2ccc(Cl)cc2)C(=O)N3c2ccc(Cl)cc2)c1 ; N#Cc1cccc([C@@]23CCCC[C@@H]2N(c2ccc(Cl)cc2)C(=O)N3c2ccc(Cl)cc2)c1]

Compounds 14B and 14C were synthesized by employing the procedures described for Compounds 12B and 12C using Compounds 14A and 14B in lieu of Compounds 12A and 12B.

Compound 14B. LC-MS (ESI) m/z: 240 [M+H]⁺.

Compound 14C. LC-MS (ESI) m/z: 254 [M+H]⁺; ¹H-NMR (CDCl₃, 400 MHz): δ (ppm) 1.71-1.75 (m, 4H), 1.82-1.88 (m, 2H), 2.21-2.28 (m, 2H), 3.59 (s, 3H), 4.03 (s, 1H), 6.36 (d, J=8.8 Hz, 2H), 7.01 (d, J=8.8 Hz, 2H).

A mixture of Compound 14C (1 g, 3.95 mmol) and 1-chloro-4-isocyanatobenzene (634 mg, 4.15 mmol) in toluene (20 mL) was heated at 100° C. for 48 hours. The mixture was concentrated and the residue was purified with flash column chromatography on silica gel (ethyl acetate in petroleum ether, 20% v/v) and preparative HPLC to yield Compound 14D. LC-MS (ESI) m/z: 375 [M+H]⁺. ¹H-NMR (CDCl₃, 400 MHz): δ (ppm) 1.53-1.62 (m, 2H), 1.83-1.93 (m, 2H), 2.03-2.09 (m, 2H), 2.25-2.32 (m, 2H), 7.26-7.29 (m, 2H), 7.43-7.48 (m, 6H).

Compound 14E was synthesized by employing the procedure described for Compound 6D using 3-bromobenzonitrile and Compounds 14D in lieu of 1-bromo-3-(trifluoromethyl)benzene and Compound 6C. LC-MS (ESI) m/z: 478 [M+H]⁺. H NMR (CDCl₃, 400 MHz): δ (ppm) 0.44-0.53 (m, 1H), 1.09-1.18 (m, 2H), 1.36-1.45 (m, 1H), 1.63-1.69 (m, 2H), 1.96-2.03 (m, 1H), 2.29-2.37 (m, 1H), 3.93 (s, 1H), 7.17 (d, J=9.2 Hz, 2H), 7.24 (s, 2H), 7.34 (d, J=8.8 Hz, 2H), 7.38-7.41 (m, 3H), 7.61 (d, J=8.0 Hz, 2H), 7.88 (s, 1H).

To a solution of Compound 14E (300 mg, 0.63 mmol) and triethylsilane (777 mg, 3.14 mmol) in DCM (10 mL) at 0° C. was added BF₃-Et₂O (178 mg, 1.26 mmol) and the mixture was stirred at 0° C. for 1 hour. The reaction mixture was concentrated under reduced pressure to give a crude product, which was purified with preparative HPLC to furnish Compound 14. LC-MS (ESI) m/z: 462 [M+H]⁺. ¹H-NMR (CDCl₃, 400 MHz): δ (ppm) 1.44-1.88 (m, 6H), 2.24-2.39 (m, 2H), 4.17 (t, J=5.2 Hz, 1H), 6.98 (d, J=8.8 Hz, 2H), 7.22 (d, J=8.8 Hz, 2H), 7.31 (d, J=9.2 Hz, 2H), 7.38 (d, J=8.8 Hz, 2H), 7.51-7.55 (m, 1H), 7.65-7.67 (m, 1H), 7.74-7.77 (m, 2H). Compound 14 was separated with chiral HPLC to yield Compound 14-1 and Compound 14-2.

Compound 14-1: LC-MS (ESI) m/z: 462 [M+H]⁺; ¹H-NMR (CDCl₃, 400 MHz): δ (ppm) 1.44-1.52 (m, 2H), 1.62-1.65 (m, 2H), 1.72-1.80 (m, 1H), 1.85-1.93 (m, 1H), 2.26-2.39 (m, 2H), 4.16 (t, J=4.8 Hz, 1H), 6.99 (d, J=8.8 Hz, 2H), 7.22 (d, J=8.8 Hz, 2H), 7.31 (d, J=8.8 Hz, 2H), 7.39 (d, J=9.2 Hz, 2H), 7.51-7.55 (m, 1H), 7.65-7.67 (m, 1H), 7.73-7.77 (m, 2H). Chiral separation conditions: MeOH contained 0.2% Methanol Ammonia; Cellulose-SC (100×4.6 mm, 5 μm); retention time: 1.56 minutes.

Compound 14-2: LC-MS (ESI) m/z: 462 [M+H]⁺; ¹H-NMR (CDCl₃, 400 MHz): δ (ppm) 1.44-1.52 (m, 2H), 1.62-1.66 (m, 2H), 1.74-1.80 (m, 1H), 1.85-1.93 (m, 1H), 2.26-2.39 (m, 2H), 4.16 (t, J=5.2 Hz, 1H), 6.99 (d, J=9.2 Hz, 2H), 7.22 (d, J=8.8 Hz, 2H), 7.31 (d, J=8.8 Hz, 2H), 7.39 (d, J=8.8 Hz, 2H), 7.51-7.55 (m, 1H), 7.65-7.67 (m, 1H), 7.73-7.77 (m, 2H). Chiral separation conditions: MeOH contained 0.2% Methanol Ammonia; Cellulose-SC (100×4.6 mm, 5 μm); retention time: 2.36 minutes.

### Example 15

### Synthesis of 1,3-bis(4-chlorophenyl)-3a-(3-(trifluoromethoxy)phenyl) octahydro-2H-benzo[d]imidazol-2-one (15-1), 1,3-bis(4-chlorophenyl)-4-(3-(trifluoromethoxy)phenyl)-1,3-diazaspiro[4.4]nonan-2-one (15), (R)-1,3-bis(4-chlorophenyl)-4-(3-(trifluoromethoxy)phenyl)-1,3-diazaspiro[4.4]nonan-2-one (15-2), and (S)-1,3-bis(4-chlorophenyl)-4-(3-(trifluoromethoxy)phenyl)-1,3-diazaspiro[4.4]nonan-2-one (15-3)

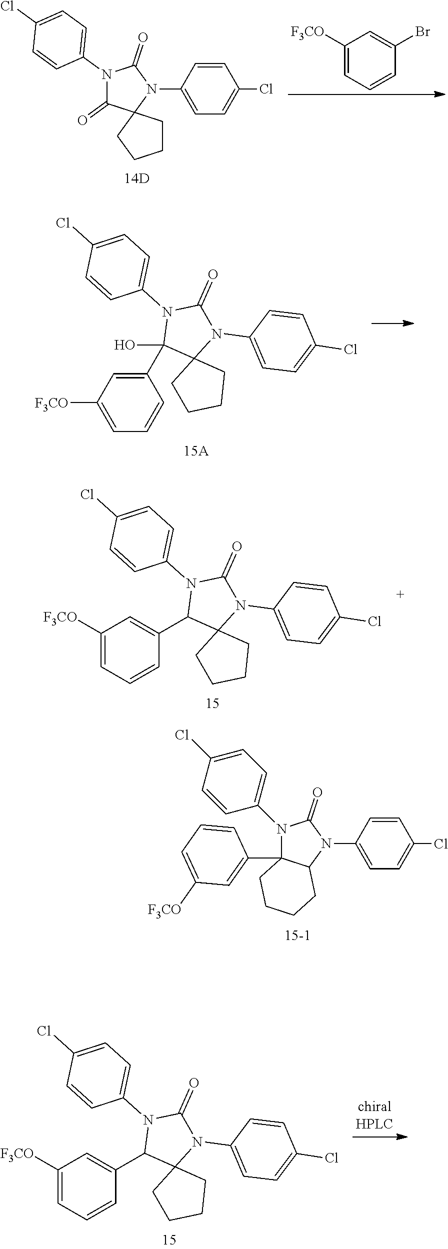 [SMILES: O=C1N(c2ccc(Cl)cc2)C(=O)C2(CCCC2)N1c1ccc(Cl)cc1 ; FC(F)(F)Oc1cccc(Br)c1 ; O=C1N(c2ccc(Cl)cc2)C2(CCCC2)C(O)(c2cccc(OC(F)(F)F)c2)N1c1ccc(Cl)cc1 ; O=C1N(c2ccc(Cl)cc2)C(c2cccc(OC(F)(F)F)c2)C2(CCCC2)N1c1ccc(Cl)cc1 ; O=C1N(c2ccc(Cl)cc2)C2CCCCC2(c2cccc(OC(F)(F)F)c2)N1c1ccc(Cl)cc1 ; O=C1N(c2ccc(Cl)cc2)C(c2cccc(OC(F)(F)F)c2)C2(CCCC2)N1c1ccc(Cl)cc1]

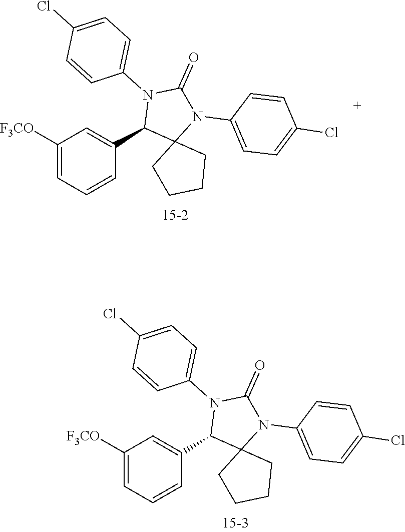 [SMILES: O=C1N(c2ccc(Cl)cc2)[C@H](c2cccc(OC(F)(F)F)c2)C2(CCCC2)N1c1ccc(Cl)cc1 ; O=C1N(c2ccc(Cl)cc2)[C@@H](c2cccc(OC(F)(F)F)c2)C2(CCCC2)N1c1ccc(Cl)cc1]

Compounds 15A, 15-1, and 15 were synthesized by employing the procedures described for Compounds 6D and 6 using 1-bromo-3-(trifluoromethoxy)benzene, Compounds 14D, and 15A at room temperature in lieu of 1-bromo-3-(trifluoromethyl)benzene, Compounds 6C, and 6D at 10° C.

Compound 15A. LC-MS (ESI) m/z: 537 [M+H]⁺. H NMR (CDCl₃, 400 MHz): δ (ppm) 0.45-0.52 (m, 1H), 1.09-1.16 (m, 2H), 1.37-1.43 (m, 1H), 1.63-1.64 (m, 2H), 1.91-1.99 (m, 1H), 2.32-2.39 (m, 1H), 3.91 (s, 1H), 7.15-7.17 (m, 4H), 7.20-7.23 (m, 2H), 7.30-7.41 (m, 6H).

Compound 15-1. LC-MS (ESI) m/z: 521 [M+H]⁺. H NMR (CDCl₃, 400 MHz): δ (ppm) 1.17-1.29 (m, 1H), 1.78-1.83 (m, 3H), 2.10-2.26 (m, 3H), 2.57-2.61 (m, 1H), 5.17 (t, J=4.0 Hz, 1H), 6.89-6.92 (m, 3H), 7.03 (d, J=8.0 Hz, 1H), 7.14 (d, J=8.0 Hz, 1H), 7.29-7.33 (m, 3H), 7.46 (s, 4H).

Compound 15 was separated with chiral HPLC to afford Compound 15-2 and 15-3.

Compound 15-2: LC-MS (ESI) m/z: 521 [M+H]⁺; ¹H-NMR (CD₃OD, 400 MHz): δ (ppm) 1.22-1.49 (m, 4H), 1.58-1.66 (m, 1H), 1.78-1.87 (m, 1H), 2.02-2.19 (m, 2H), 5.33 (s, 1H), 7.22-7.33 (m, 6H), 7.41-7.43 (m, 1H), 7.48-7.54 (m, 5H). Chiral separation conditions: MeOH contained 0.2% Methanol Ammonia; EnantioPak AD (100×4.6 mm, 5 μm); retention time: 1.24 minutes.

Compound 15-3: LC-MS (ESI) m/z: 521 [M+H]⁺; ¹H-NMR (CD₃OD, 400 MHz): δ (ppm) 1.11-1.39 (m, 4H), 1.49-1.57 (m, 1H), 1.69-1.76 (m, 1H), 1.91-2.09 (m, 2H), 5.22 (s, 1H), 7.11-7.22 (m, 6H), 7.31-7.32 (m, 1H), 7.38-7.43 (m, 5H). Chiral separation conditions: MeOH contained 0.2% Methanol Ammonia; EnantioPak AD (100×4.6 mm, 5 μm); retention time: 1.95 minutes.

### Example 16

### Synthesis of 1,3-bis(4-chlorophenyl)-3a-(3-(difluoromethyl)phenyl)hexahydro cyclopenta[d]imidazol-2(1H)-one (16), (3aS,6aS)-1,3-bis(4-chlorophenyl)-3a-(3-(difluoromethyl)phenyl) hexahydrocyclopenta[d]imidazol-2(1H)-one (16-1), and (3aR,6aR)-1,3-bis(4-chlorophenyl)-3a-(3-(difluoromethyl)phenyl)hexahydrocyclopenta[d]imidazol-2(1H)-one (16-2)

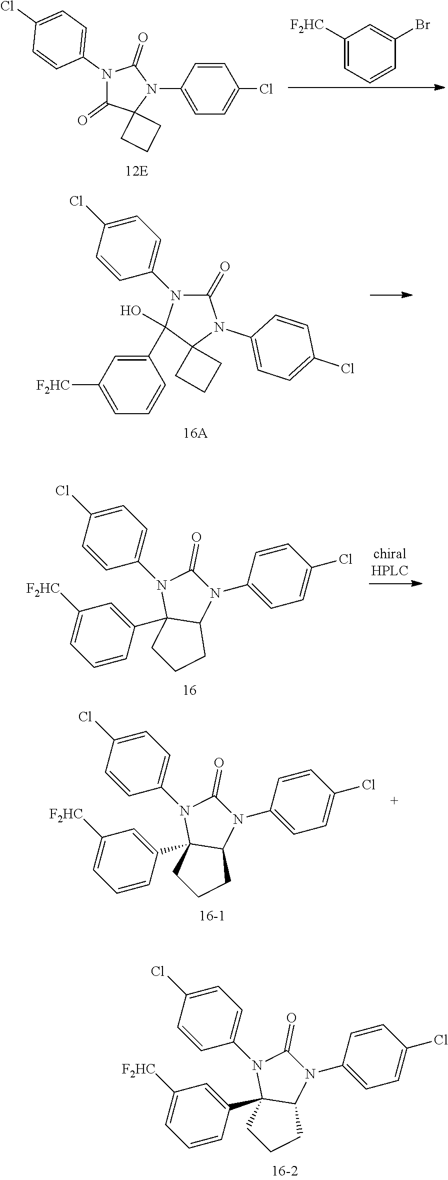 [SMILES: O=C1N(c2ccc(Cl)cc2)C(=O)C2(CCC2)N1c1ccc(Cl)cc1 ; FC(F)c1cccc(Br)c1 ; O=C1N(c2ccc(Cl)cc2)C2(CCC2)C(O)(c2cccc(C(F)F)c2)N1c1ccc(Cl)cc1 ; O=C1N(c2ccc(Cl)cc2)C2CCCC2(c2cccc(C(F)F)c2)N1c1ccc(Cl)cc1 ; O=C1N(c2ccc(Cl)cc2)[C@H]2CCC[C@@]2(c2cccc(C(F)F)c2)N1c1ccc(Cl)cc1 ; O=C1N(c2ccc(Cl)cc2)[C@@H]2CCC[C@]2(c2cccc(C(F)F)c2)N1c1ccc(Cl)cc1]

Compounds 16A and 16 were synthesized by employing the procedures described for Compounds 6D and 6 using 1-bromo-3-(difluoromethyl)benzene, Compounds 12E, and 16A at room temperature in lieu of 1-bromo-3-(trifluoromethyl)benzene, Compounds 6C, and 6D at 10° C.

Compound 16A. LC-MS (ESI) m/z: 489 [M+H]⁺. ¹H-NMR (CDCl₃, 400 MHz): δ (ppm) 0.76-0.90 (m, 1H), 1.04-1.16 (m, 1H), 1.70-1.79 (m, 1H), 1.96-2.04 (m, 1H), 2.20-2.30 (m, 1H), 2.69-2.79 (m, 1H), 3.67-3.72 (m, 1H), 6.42-6.80 (m, 1H), 6.87-7.07 (m, 1H), 7.12-7.26 (m, 3H), 7.30-7.51 (m, 7H), 7.52-7.60 (m, 1H). Compound 16 was separated with chiral HPLC to afford Compound 16-1 and Compound 16-2.

Compound 16-1: LC-MS (ESI) m/z: 473 [M+H]⁺; ¹H-NMR (CDCl₃, 400 MHz): δ (ppm) 1.90-2.08 (m, 3H), 2.10-2.24 (m, 1H), 2.27-2.35 (m, 1H), 2.40-2.50 (m, 1H), 4.36-4.41 (m, 1H), 6.49-6.81 (m, 1H), 6.94-7.02 (m, 2H), 7.16-7.22 (m, 2H), 7.28-7.34 (m, 2H), 7.46-7.62 (m, 6H). Chiral separation conditions: MeOH contained 0.2% methanol ammonia; OJ-H (4.6×250 mm, 5 μm); retention time: 1.56 minutes.

Compound 16-2: LC-MS (ESI) m/z: 473 [M+H]⁺; ¹H-NMR (CDCl₃, 400 MHz): δ (ppm) 1.90-2.08 (m, 3H), 2.10-2.24 (m, 1H), 2.26-2.37 (m, 1H), 2.40-2.50 (m, 1H), 4.36-4.41 (m, 1H), 6.48-6.80 (m, 1H), 6.94-7.02 (m, 2H), 7.16-7.22 (m, 2H), 7.28-7.34 (m, 2H), 7.46-7.64 (m, 6H). Chiral separation conditions: MeOH contained 0.2% methanol ammonia; OJ-H (4.6×250 mm, 5 μm); retention time: 1.95 minutes.

### Example 17

### Synthesis of 1,3-bis(4-chlorophenyl)-4,4-diethyl-5-(3-(trifluoromethoxy) phenyl)imidazolidin-2-one (17), (R)-1,3-bis(4-chlorophenyl)-4,4-diethyl-5-(3-(trifluoromethoxy)phenyl)imidazolidin-2-one (17-1), and (S)-1,3-bis(4-chlorophenyl)-4,4-diethyl-5-(3-(trifluoromethoxy)phenyl)imidazolidin-2-one (17-2)

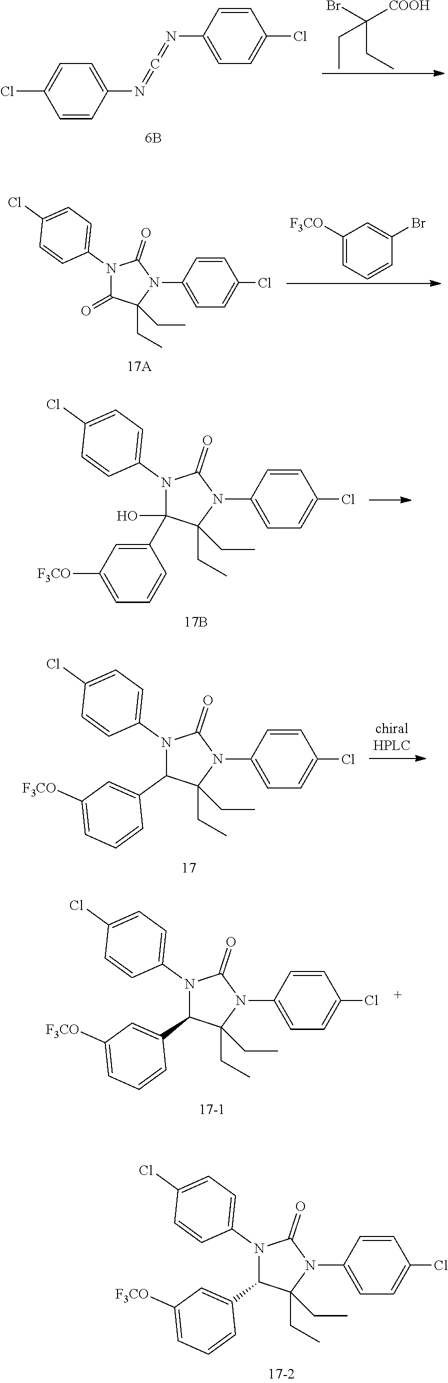

Compounds 17A, 17B, and 17 were synthesized by employing the procedures described for Compounds 6C, 6D, and 6 using 2-bromo-2-ethylbutanoic acid, Compounds 17A, 1-bromo-3-(trifluoromethoxy)benzene, and 17B at room temperature in lieu of 2-bromo-2-methylpropanoic acid, Compounds 6C, 1-bromo-3-(trifluoromethyl)benzene, and 6D at 10° C.

Compound 17A. LC-MS (ESI) m/z: 377 [M+H]⁺.

Compound 17B. LC-MS (ESI) m/z: 539 [M+H]⁺. ¹H-NMR (400 MHz, CDCl₃): δ (ppm) 0.58 (t, J=7.2 Hz, 3H), 1.09 (t, J=7.2 Hz, 3H), 1.15-1.28 (m, 1H), 1.32-1.44 (m, 1H), 1.77-1.89 (m, 1H), 2.12-2.24 (m, 1H), 3.46 (s, 1H), 7.09-7.25 (m, 7H), 7.32-7.50 (m, 5H).

Compound 17. LC-MS (ESI) m/z: 523 [M+H]⁺. ¹H-NMR (400 MHz, CDCl₃): δ (ppm) 0.48 (t, J=7.2 Hz, 3H), 1.12 (t, J=7.2 Hz, 3H), 1.27-1.34 (m, 2H), 1.79-1.87 (m, 1H), 1.88-2.08 (m, 1H), 4.96 (s, 1H), 7.16-7.22 (m, 7H), 7.36-7.42 (m, 5H).

Compound 17 was separated with chiral HPLC to afford Compound 17-1 and Compound 17-2.

Compound 17-1. LC-MS (ESI) m/z: 523 [M+H]⁺. ¹H-NMR (400 MHz, CDCl₃): δ (ppm) 0.48 (t, J=7.2 Hz, 3H), 1.12 (t, J=7.6 Hz, 3H), 1.24-1.33 (m, 2H), 1.78-1.89 (m, 1H), 1.89-2.00 (m, 1H), 4.96 (s, 1H), 7.15-7.23 (m, 6H), 7.26-7.30 (m, 1H), 7.36-7.44 (m, 5H). Chiral separation condition: MeOH contained 0.2% methanol ammonia; OJ-H (4.6×250 mm, 5 μm); retention time: 3.74 minute.

Compound 17-2. LC-MS (ESI) m/z: 523 [M+H]⁺. ¹H-NMR (400 MHz, CDCl₃): δ (ppm) 0.48 (t, J=7.2 Hz, 3H), 1.12 (t, J=7.6 Hz, 3H), 1.24-1.33 (m, 2H), 1.78-1.89 (m, 1H), 1.89-2.00 (m, 1H), 4.96 (s, 1H), 7.15-7.23 (m, 6H), 7.26-7.30 (m, 1H), 7.35-7.44 (m, 5H). Chiral separation condition: MeOH contained 0.2% methanol ammonia; OJ-H (4.6×250 mm, 5 μm); retention time: 4.48 minute.

### Example 18

### Synthesis of 1,3-bis(4-chlorophenyl)-3a-(3-hydroxyphenyl)hexahydro cyclopenta[d]imidazol-2(1H)-one (18)

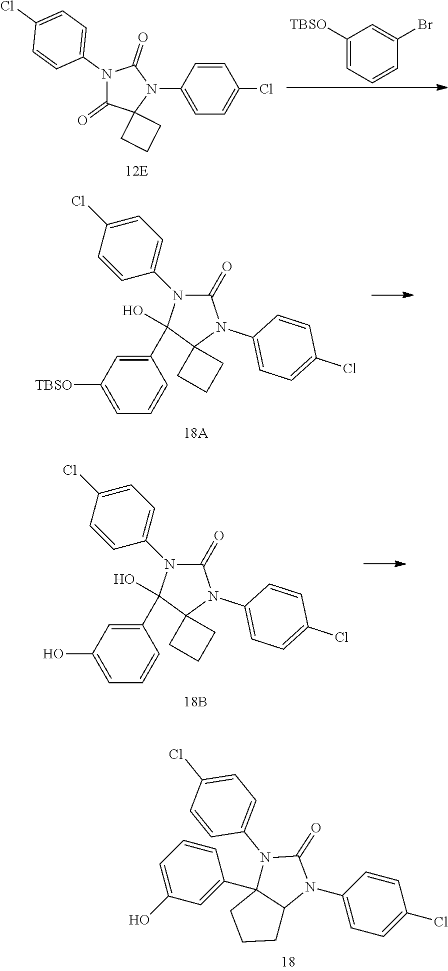 [SMILES: O=C1N(c2ccc(Cl)cc2)C(=O)C2(CCC2)N1c1ccc(Cl)cc1 ; CC(C)(C)[Si](C)(C)Oc1cccc(Br)c1 ; CC(C)(C)[Si](C)(C)Oc1cccc(C2(O)N(c3ccc(Cl)cc3)C(=O)N(c3ccc(Cl)cc3)C23CCC3)c1 ; O=C1N(c2ccc(Cl)cc2)C2(CCC2)C(O)(c2cccc(O)c2)N1c1ccc(Cl)cc1 ; O=C1N(c2ccc(Cl)cc2)C2CCCC2(c2cccc(O)c2)N1c1ccc(Cl)cc1]

Compounds 18A, 18B, and 18 were synthesized by employing the procedures described for Compounds 6D, 9B, and 14 using (3-bromophenoxy)(tert-butyl)dimethylsilane, Compounds 12E, 18A, and 18B at room temperature in lieu of 1-bromo-3-(trifluoromethyl)benzene, Compounds 6C, 9A, and 14E at 0° C.

Compound 18A. LC-MS (ESI) m/z: 569 [M+H]⁺.

Compound 18B. LC-MS (ESI) m/z: 455 [M+H]⁺. ¹H-NMR (400 MHz, CDCl₃): δ (ppm) 0.88-0.97 (m, 2H), 1.66-1.69 (m, 1H), 1.90-1.93 (m, 1H), 2.09-2.12 (m, 1H), 2.67-2.71 (m, 1H), 6.66-6.69 (m, 1H), 6.76-6.88 (m, 2H), 7.01-7.14 (m, 1H), 7.25-7.27 (m, 3H), 7.47-7.51 (m, 4H), 7.57-7.60 (m, 2H), 9.44 (s, 1H).

Compound 18. LC-MS (ESI) m/z: 439 [M+H]⁺; ¹H-NMR (CDCl₃, 400 MHz): δ (ppm) 1.87-2.15 (m, 3H), 2.03-2.27 (m, 2H), 2.34-2.45 (m, 1H), 4.35-4.37 (m, 1H), 5.35-5.37 (m, 1H), 6.79-6.94 (m, 3H), 7.01-7.03 (m, 2H), 7.16-7.18 (m, 2H), 7.23-7.26 (m, 2H), 7.26-7.28 (m, 1H), 7.55-7.58 (m, 2H).

### Example 19

### Synthesis of 1,3-bis(4-chlorophenyl)-4-ethyl-4-methyl-5-(3-(trifluoromethoxy)phenyl)imidazolidin-2-one (19), (4R,5R)-1,3-bis(4-chlorophenyl)-4-ethyl-4-methyl-5-(3-(trifluoromethoxy)phenyl)imidazolidin-2-one (19-1), (4R,5S)-1,3-bis(4-chlorophenyl)-4-ethyl-4-methyl-5-(3-(trifluoromethoxy)phenyl)imidazolidin-2-one (19-2), (4S,5S)-1,3-bis(4-chlorophenyl)-4-ethyl-4-methyl-5-(3-(trifluoromethoxy)phenyl) imidazolidin-2-one (19-3), and (4S,5R)-1,3-bis(4-chlorophenyl)-4-ethyl-4-methyl-5-(3-(trifluoromethoxy)phenyl)imidazolidin-2-one (19-4)

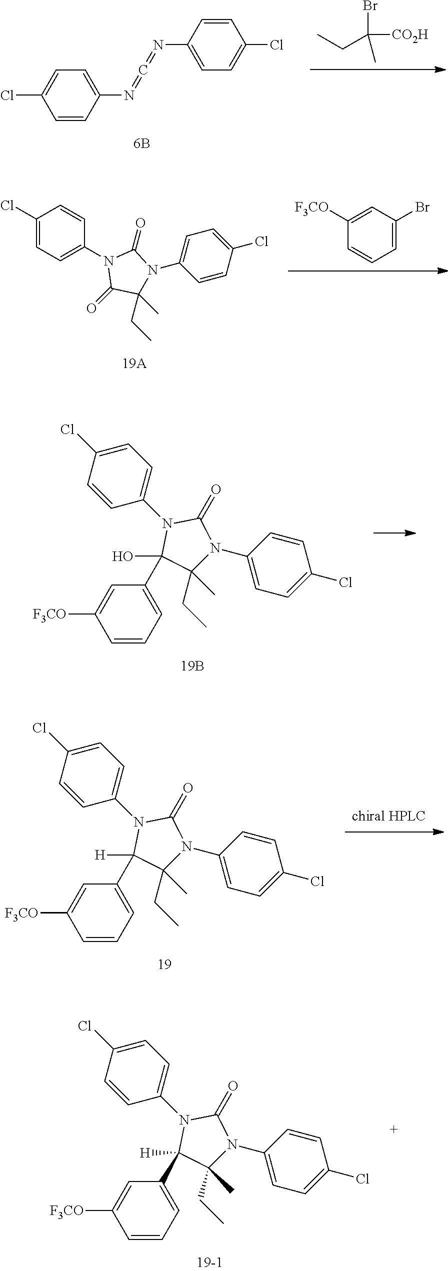

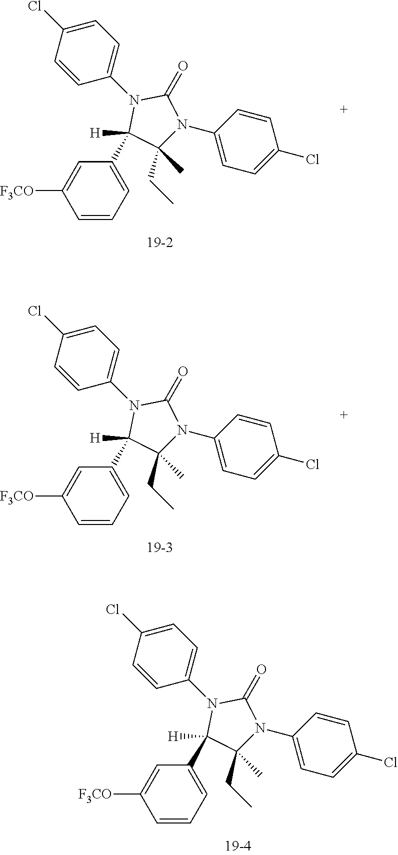 [SMILES: CC[C@]1(C)[C@H](c2cccc(OC(F)(F)F)c2)N(c2ccc(Cl)cc2)C(=O)N1c1ccc(Cl)cc1 ; CC[C@@]1(C)[C@H](c2cccc(OC(F)(F)F)c2)N(c2ccc(Cl)cc2)C(=O)N1c1ccc(Cl)cc1 ; CC[C@@]1(C)[C@@H](c2cccc(OC(F)(F)F)c2)N(c2ccc(Cl)cc2)C(=O)N1c1ccc(Cl)cc1]

Compounds 19A, 19B, and 19 were synthesized by employing the procedures described for Compounds 6C, 6D, and 6 using 2-bromo-2-methylbutanoic acid, Compounds 19A, 1-bromo-3-(trifluoromethoxy)benzene, and 19B at room temperature in lieu of 2-bromo-2-methylpropanoic acid, Compounds 6C, 1-bromo-3-(trifluoromethyl)benzene, and 6D at 10° C.

Compound 19A. LC-MS (ESI) m/z: 363 [M+H]⁺.

Compound 19B. LC-MS (ESI) m/z: 525 [M+H]⁺; ¹H-NMR (400 MHz, CDCl₃): δ (ppm) 0.86 (t, J=7.6 Hz, 3H), 0.91 (s, 3H), 1.74-1.80 (m, 1H), 1.96-2.02 (m, 1H), 3.62 (s, 1H), 7.14-7.19 (m, 5H), 7.26-7.29 (m, 2H), 7.34-7.38 (m, 5H).

Compound 19 was separated with chiral HPLC to give Compound 19-1, Compound 19-2, Compound 19-3, and Compound 19-4.

Compound 19-1: LC-MS (ESI) m/z: 509 [M+H]⁺; ¹H-NMR (400 MHz, CDCl₃): δ (ppm) 0.74 (s, 3H), 1.10 (t, J=7.6 Hz, 3H), 1.69-1.74 (m, 1H), 1.84-1.89 (m, 1H), 4.98 (s, 1H), 7.05-7.24 (m, 7H), 7.35-7.43 (m, 5H). Chiral separation conditions: MeOH contained 0.2% Methanol Ammonia; OJ-H (4.6×100 mm, 5 μm); retention time: 1.43 minutes.

Compound 19-2: LC-MS (ESI) m/z: 509 [M+H]⁺; ¹H-NMR (400 MHz, CDCl₃): δ (ppm) 0.53 (t, J=7.6 Hz, 3H), 1.32 (q, J=7.6 Hz, 2H), 1.53 (s, 3H), 4.92 (s, 1H), 7.14-7.23 (m, 6H), 7.36-7.42 (m, 6H). Chiral separation conditions: MeOH contained 0.2% Methanol Ammonia; OJ-H (4.6×100 mm, 5 μm); retention time: 1.99 minutes.

Compound 19-3: LC-MS (ESI) m/z: 509 [M+H]⁺; ¹H-NMR (400 MHz, CDCl₃): δ (ppm) 0.74 (s, 3H), 1.10 (t, J=7.6 Hz, 3H), 1.65-1.74 (m, 1H), 1.82-1.92 (m, 1H), 4.98 (s, 1H), 7.12-7.24 (m, 7H), 7.37-7.43 (m, 5H). Chiral separation conditions: MeOH contained 0.2% Methanol Ammonia; OJ-H (4.6×100 mm, 5 μm); retention time: 1.52 minutes.

Compound 19-4: LC-MS (ESI) m/z: 509 [M+H]⁺; ¹H-NMR (400 MHz, CDCl₃): δ (ppm) 0.53 (t, J=7.6 Hz, 3H), 1.32 (q, J=7.6 Hz, 2H), 1.53 (s, 3H), 4.92 (s, 1H), 7.17-7.23 (m, 6H), 7.36-7.42 (m, 6H). Chiral separation conditions: MeOH contained 0.2% Methanol Ammonia; OJ-H (4.6×100 mm, 5 μm); retention time: 2.2 minutes.

### Example 20

### Synthesis of 4,4-dimethyl-5-(3-(trifluoromethoxy)phenyl)-1,3-bis(4-(trifluoromethyl)phenyl)imidazolidin-2-one (20), (R)-4,4-dimethyl-5-(3-(trifluoromethoxy) phenyl)-1,3-bis(4-(trifluoromethyl)phenyl)imidazolidin-2-one (20-1), and (S)-4,4-dimethyl-5-(3-(trifluoromethoxy)phenyl)-1,3-bis(4-(trifluoromethyl)phenyl) imidazolidin-2-one (20-2)

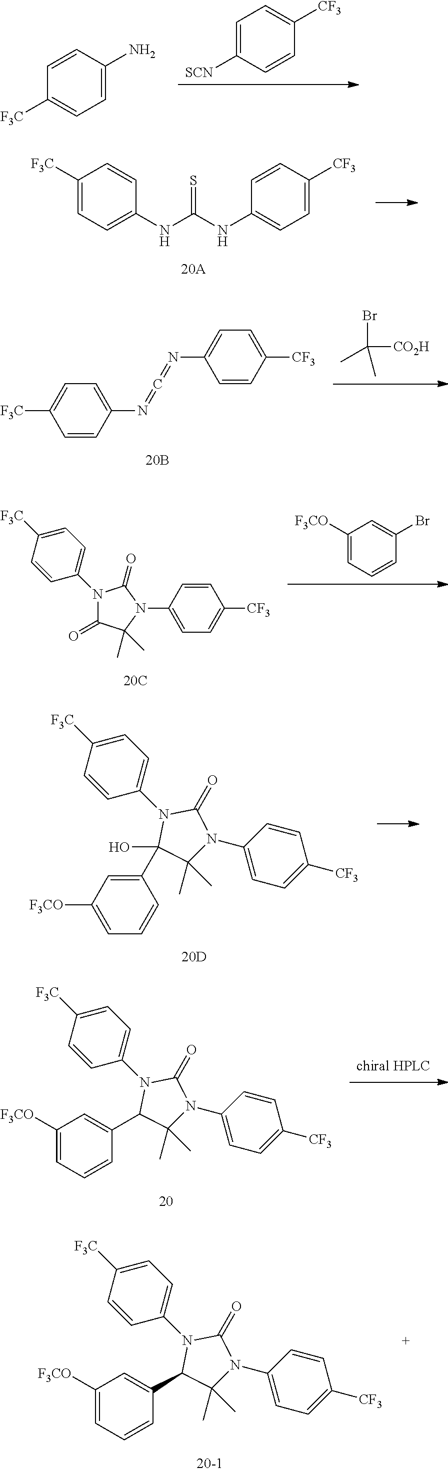

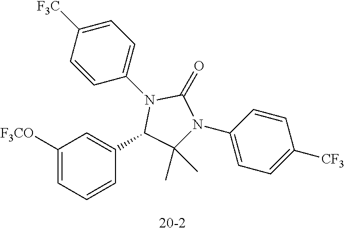 [SMILES: CC1(C)[C@H](c2cccc(OC(F)(F)F)c2)N(c2ccc(C(F)(F)F)cc2)C(=O)N1c1ccc(C(F)(F)F)cc1]

Compounds 20A, 20B, 20C, 20D, and 20 were synthesized by employing the procedures described for Compounds 6A, 6B, 6C, 6D, and 6 using 1-isothiocyanato-4-(trifluoromethyl)benzene, 4-(trifluoromethyl)aniline, Compounds 20A, 20B, 20C, 1-bromo-3-(trifluoromethoxy)benzene, and 20D at room temperature in lieu of 1-chloro-4-isothiocyanatobenzene, 4-chloroaniline, Compounds 6A, 6B, 6C, 1-bromo-3-(trifluoromethyl)benzene, and 6D at 10° C.

Compound 20A. LC-MS (ESI) m/z: 365 [M+H]⁺; ¹H-NMR (DMSO-d₆, 400 MHz): δ (ppm) 7.69-7.77 (m, 8H), 10.37 (s, 2H).

Compound 20B. LC-MS (ESI) m/z: non-ionizable compound under routine conditions used; ¹H-NMR (CDCl₃, 400 MHz): δ (ppm) 7.28 (d, J=8.4 Hz, 4H), 7.61 (d, J=8.4 Hz, 4H).

Compound 20C. LC-MS (ESI) m/z: 417 [M+H]⁺; ¹H-NMR (CD₃OD, 400 MHz): δ (ppm) 1.61 (s, 6H), 7.68-7.70 (m, 2H), 7.77-7.79 (m, 2H), 7.83-7.85 (m, 4H).

Compound 20D. LC-MS (ESI) m/z: 579 [M+H]⁺; ¹H-NMR (CD₃OD, 400 MHz): δ (ppm) 0.92 (s, 3H), 1.41 (s, 3H), 7.23-7.25 (m, 1H), 7.37-7.55 (m, 5H), 7.64-7.66 (m, 2H), 7.75-7.82 (m, 4H).

Compound 20 was separated with chiral HPLC to afford Compound 20-1 and Compound 20-2.

Compound 20-1: LC-MS (ESI) m/z: 563 [M+H]⁺; ¹H-NMR (CD₃OD, 400 MHz): δ (ppm) 0.87 (s, 3H), 1.62 (s, 3H), 5.46 (s, 1H), 7.26-7.55 (m, 8H), 7.52-7.55 (m, 2H), 7.77-7.79 (m, 2H). Chiral separation conditions: MeOH contained 0.2% Methanol Ammonia; S,S-Whelk-Ol (4.6×100 mm, 5 μm); retention time: 2.46 minutes.

Compound 20-2: LC-MS (ESI) m/z: 563 [M+H]⁺; ¹H-NMR (CD₃OD, 400 MHz): δ (ppm) 0.76 (s, 3H), 1.50 (s, 3H), 5.35 (s, 1H), 7.14-7.43 (m, 8H), 7.57-7.59 (m, 2H), 7.66-7.68 (m, 2H). Chiral separation conditions: MeOH contained 0.2% Methanol Ammonia, S,S-Whelk-Ol (4.6×100 mm, 5 μm); retention time: 1.20 minutes.

### Example 21

### Synthesis of 4,4-dimethyl-1,3-di-p-tolyl-5-(3-(trifluoromethoxy)phenyl) imidazolidin-2-one (21), (R)-4,4-dimethyl-1,3-di-p-tolyl-5-(3-(trifluoromethoxy)phenyl) imidazolidin-2-one (21-1) and (S)-4,4-dimethyl-1,3-di-p-tolyl-5-(3-(trifluoromethoxy) phenyl)imidazolidin-2-one (21-2)

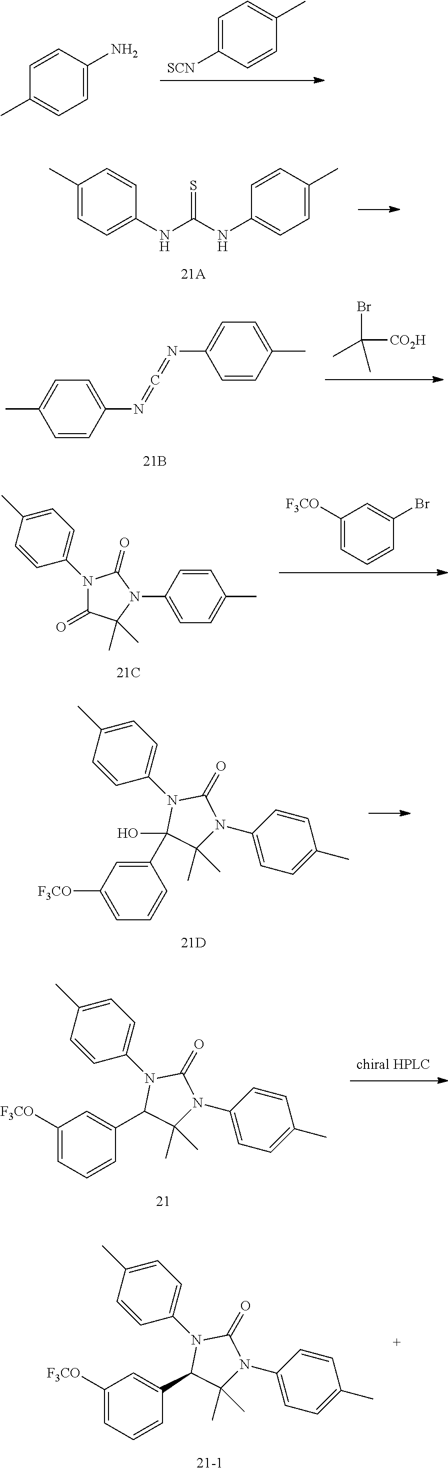

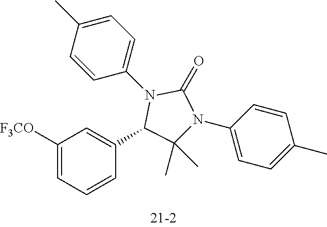 [SMILES: Cc1ccc(N2C(=O)N(c3ccc(C)cc3)C(C)(C)[C@@H]2c2cccc(OC(F)(F)F)c2)cc1]

Compounds 21A, 21B, 21C, 21D, and 21 were synthesized by employing the procedures described for Compounds 6A, 6B, 6C, 6D, and 6 using 1-isothiocyanato-4-methylbenzene, p-toluidine, Compounds 21A, 21B, 21C, 1-bromo-3-(trifluoromethoxy)benzene, and 21D at room temperature in lieu of 1-chloro-4-isothiocyanatobenzene, 4-chloroaniline, Compounds 6A, 6B, 6C, 1-bromo-3-(trifluoromethyl)benzene, and 6D at 10° C.

Compound 21A. LC-MS (ESI) m/z: 257 [M+H]⁺; ¹H-NMR (CDCl₃, 400 MHz): δ (ppm) 2.36 (s, 6H), 7.19-7.26 (m, 8H), 7.70 (s, 2H).

Compound 21B. ¹H-NMR (CDCl₃, 400 MHz): δ (ppm) 2.33 (s, 6H), 7.06 (d, J=8.0 Hz, 4H), 7.12 (d, J=8.0 Hz, 4H).

Compound 21C. LC-MS (ESI) m/z: 309 [M+H]⁺; ¹H-NMR (CDCl₃, 400 MHz): δ (ppm) 1.52 (s, 6H), 2.38 (s, 3H), 2.40 (s, 3H), 7.19 (d, J=8.0 Hz, 2H), 7.25-7.28 (m, 4H), 7.36 (d, J=8.0 Hz, 2H).

Compound 21D. LC-MS (ESI) m/z: 471 [M+H]⁺; ¹H-NMR (CDCl₃, 400 MHz): δ (ppm) 0.85 (s, 3H), 1.36 (s, 3H), 2.25 (s, 3H), 2.38 (s, 3H), 3.21 (s, 1H), 7.03 (d, J=8.0 Hz, 2H), 7.14-7.24 (m, 5H), 7.31-7.42 (m, 5H).

Compound 21 was separated with chiral HPLC to afford Compound 21-1 and Compound 21-2.

Compound 21-1: LC-MS (ESI) m/z: 455 [M+H]⁺; ¹H-NMR (CDCl₃, 400 MHz): δ (ppm) 0.77 (s, 3H), 1.50 (s, 3H), 2.24 (s, 3H), 2.37 (s, 3H), 4.97 (s, 1H), 7.03 (d, J=8.0 Hz, 2H), 7.11-7.24 (m, 7H), 7.30-7.39 (m, 3H). Chiral separation conditions: Methanol contained 0.2% methanol ammonia; IC (4.6×100 mm, 5 μm); 1.68 minutes.

Compound 21-2: LC-MS (ESI) m/z: 455 [M+H]⁺; ¹H-NMR (CDCl₃, 400 MHz): δ (ppm) 0.77 (s, 3H), 1.50 (s, 3H), 2.24 (s, 3H), 2.37 (s, 3H), 4.97 (s, 1H), 7.03 (d, J=8.0 Hz, 2H), 7.11-7.24 (m, 7H), 7.30-7.39 (m, 3H). Chiral separation conditions: Methanol contained 0.2% methanol ammonia; IC (4.6×100 mm, 5 μm); 2.51 minutes.

### Example 22

### Synthesis of (4R)-1,3-bis(4-chlorophenyl)-4-methyl-4-propyl-5-(3-(trifluoromethoxy)phenyl)imidazolidin-2-one (22-1), (4R,5R)-1,3-bis(4-chlorophenyl)-4-methyl-4-propyl-5-(3-(trifluoromethoxy)phenyl)imidazolidin-2-one (22-2), (4R,5S)-1,3-bis(4-chlorophenyl)-4-methyl-4-propyl-5-(3-(trifluoromethoxy)phenyl)imidazolidin-2-one (22-3), (4S)-1,3-bis(4-chlorophenyl)-4-methyl-4-propyl-5-(3-(trifluoromethoxy)phenyl) imidazolidin-2-one (22-4), and (4S,5S)-1,3-bis(4-chlorophenyl)-4-methyl-4-propyl-5-(3-(trifluoromethoxy)phenyl)imidazolidin-2-one (22-5)

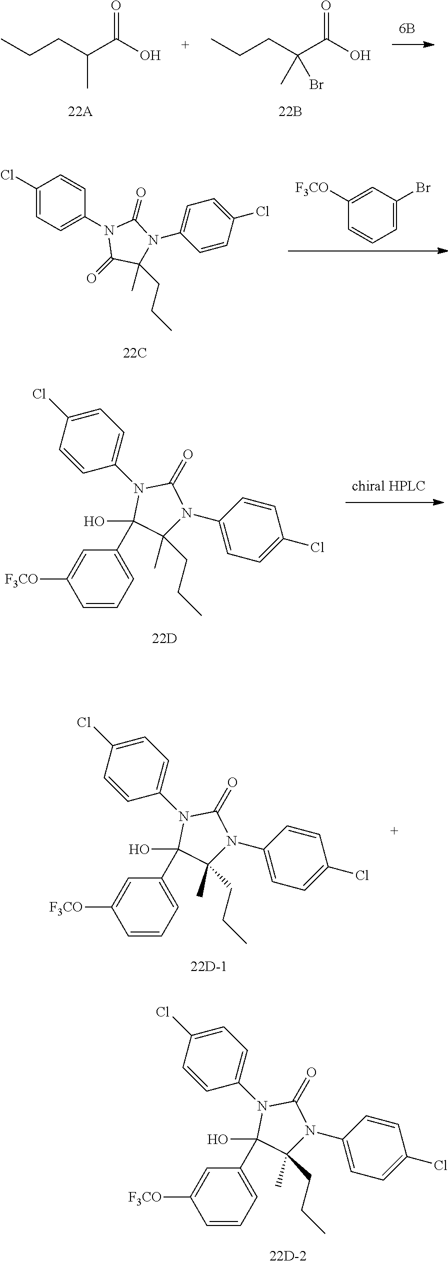

A mixture of 2-methylpentanoic acid 22A (5.8 g, 50 mmol) and NBS (9.3 g, 52.5 mmol) in CCl₄ (50 mL) was heated at reflux under nitrogen overnight. The resulting white solid (succinimide) was filtered off. The filtrate was concentrated to give a crude product, which was distilled under reduced pressure to afford Compound 22B. ¹H-NMR (400 MHz, CDCl₃): δ (ppm) 0.98 (t, J=7.6 Hz, 3H), 1.37-1.53 (m, 2H), 1.91 (s, 3H), 2.09-2.13 (m, 2H).

Compounds 22C and 22D were synthesized by employing the procedures described for Compounds 6C and 6D using Compounds 22B, 1-bromo-3-(trifluoromethoxy)benzene, and 22C in lieu of 2-bromo-2-methylpropanoic acid, 1-bromo-3-(trifluoromethyl)benzene, and Compound 6C.

Compound 22C. LC-MS (ESI) m/z: 377 [M+H]⁺.

Compound 22D. LC-MS (ESI) m/z: 539 [M+H]⁺. ¹H-NMR (400 MHz, CDCl₃): δ (ppm) 0.87 (t, J=6.8 Hz, 3H), 0.92 (s, 3H), 1.03-1.07 (m, 1H), 1.38-1.44 (m, 1H), 1.64-1.72 (m, 1H), 1.85-1.93 (m, 1H), 3.43 (s, 1H), 7.15-7.20 (m, 5H), 7.27-7.30 (m, 2H), 7.33-7.40 (m, 5H).

Compound 22D (500 mg) was separated with chiral HPLC to give Compound 22D-1 and Compound 22D-2.

Compound 22D-1: LC-MS (ESI) m/z: 539 [M+H]⁺. ¹H-NMR (400 MHz, CDCl₃): δ (ppm) 0.86 (t, J=6.8 Hz, 3H), 0.91 (s, 3H), 1.01-1.05 (m, 1H), 1.35-1.44 (m, 1H), 1.62-1.70 (m, 1H), 1.84-1.92 (m, 1H), 3.60 (s, 1H), 7.13-7.19 (m, 5H), 7.28-7.29 (m, 2H), 7.31-7.39 (m, 5H). Chiral separation conditions: MeOH contained 0.2% Methanol Ammonia; Cellulose-SC (4.6×100 mm, 5 μm); retention time: 1.04 minutes.

Compound 22D-2: LC-MS (ESI) m/z: 539 [M+H]⁺. ¹H-NMR (400 MHz, CDCl₃): δ (ppm) 0.86 (t, J=6.8 Hz, 3H), 0.91 (s, 3H), 0.99-1.08 (m, 1H), 1.37-1.44 (m, 1H), 1.63-1.70 (m, 1H), 1.84-1.92 (m, 1H), 3.58 (s, 1H), 7.14-7.19 (m, 5H), 7.28-7.30 (m, 2H), 7.34-7.39 (m, 5H). Chiral separation conditions: MeOH contained 0.2% Methanol Ammonia; Cellulose-SC (4.6×100 mm, 5 μm); retention time: 2.11 minutes.

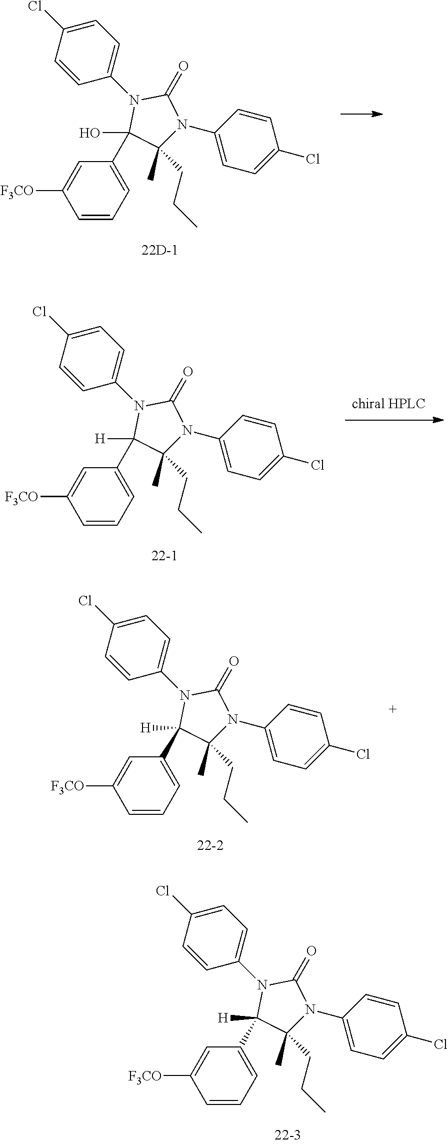 [SMILES: CCC[C@@]1(C)N(c2ccc(Cl)cc2)C(=O)N(c2ccc(Cl)cc2)C1(O)c1cccc(OC(F)(F)F)c1 ; CCC[C@]1(C)C(c2cccc(OC(F)(F)F)c2)N(c2ccc(Cl)cc2)C(=O)N1c1ccc(Cl)cc1 ; CCC[C@]1(C)[C@@H](c2cccc(OC(F)(F)F)c2)N(c2ccc(Cl)cc2)C(=O)N1c1ccc(Cl)cc1 ; CCC[C@]1(C)[C@H](c2cccc(OC(F)(F)F)c2)N(c2ccc(Cl)cc2)C(=O)N1c1ccc(Cl)cc1]

Compound 22-1 was synthesized by employing the procedure described for Compound 6 using Compound 22D-1 at room temperature in lieu of Compound 6D at 10° C. Compound 22-1 was separated with chiral HPLC to give Compound 22-2 and Compound 22-3.

Compound 22-2: LC-MS (ESI) m/z: 523 [M+H]⁺; ¹H-NMR (400 MHz, CDCl₃): δ (ppm) 0.49 (t, J=7.6 Hz, 3H), 0.86-0.90 (m, 1H), 1.02-1.15 (m, 2H), 1.23-1.31 (m, 1H), 1.52 (s, 3H), 4.91 (s, 1H), 7.16-7.23 (m, 6H), 7.29-7.31 (m, 1H), 7.36-7.42 (m, 5H). Chiral separation conditions: MeOH contained 0.2% Methanol Ammonia; IC (4.6×100 mm, m); retention time: 1.32 minutes.

Compound 22-3: LC-MS (ESI) m/z: 523 [M+H]⁺; ¹H-NMR (400 MHz, CDCl₃): δ (ppm) 0.50 (t, J=7.6 Hz, 3H), 0.86-0.91 (m, 1H), 1.01-1.16 (m, 2H), 1.23-1.31 (m, 1H), 1.52 (s, 3H), 4.91 (s, 1H), 7.16-7.23 (m, 6H), 7.29-7.31 (m, 1H), 7.36-7.42 (m, 5H). Chiral separation conditions: MeOH contained 0.2% Methanol Ammonia; IC (4.6×100 mm, m); retention time: 1.72 minutes.

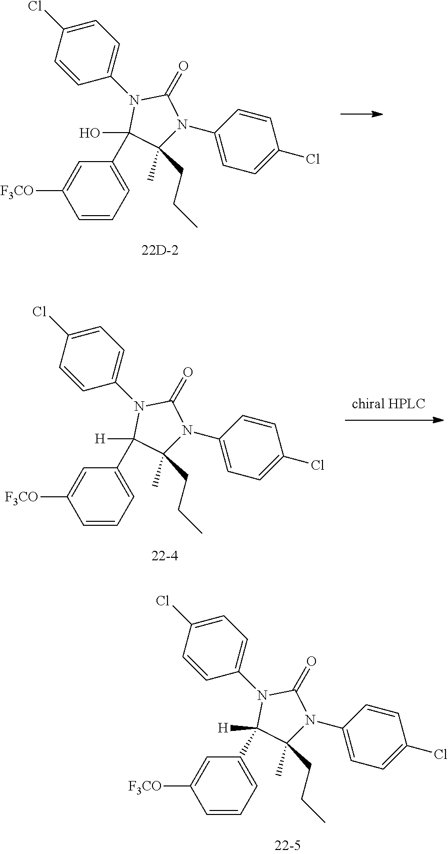 [SMILES: CCC[C@]1(C)N(c2ccc(Cl)cc2)C(=O)N(c2ccc(Cl)cc2)C1(O)c1cccc(OC(F)(F)F)c1 ; CCC[C@@]1(C)C(c2cccc(OC(F)(F)F)c2)N(c2ccc(Cl)cc2)C(=O)N1c1ccc(Cl)cc1 ; CCC[C@@]1(C)[C@H](c2cccc(OC(F)(F)F)c2)N(c2ccc(Cl)cc2)C(=O)N1c1ccc(Cl)cc1]

Compound 22-4 were synthesized by employing the procedure described for Compound 6 using Compound 22D-2 at room temperature in lieu of Compound 6D at 10° C. Compound 22-4 was separated with chiral HPLC to give Compound 22-5. LC-MS (ESI) m/z: 523 [M+H]⁺; ¹H-NMR (400 MHz, CDCl₃): δ (ppm) 0.50 (t, J=7.6 Hz, 3H), 0.86-0.89 (m, 1H), 1.02-1.16 (m, 2H), 1.23-1.29 (m, 1H), 1.52 (s, 3H), 4.91 (s, 1H), 7.16-7.23 (m, 6H), 7.29-7.31 (m, 1H), 7.36-7.42 (m, 5H). Chiral separation conditions: MeOH contained 0.2% Methanol Ammonia; IC (4.6×100 mm, 5 μm); retention time: 1.38 minutes.

### Example 23

### Synthesis of 1,3-bis(4-chlorophenyl)-3a-(3-(difluoromethoxy)phenyl) hexahydrocyclopenta[d]imidazol-2(1H)-one (23), (3aS,6aS)-1,3-bis(4-chlorophenyl)-3a-(3-(difluoromethoxy)phenyl)hexahydrocyclopenta [d]imidazol-2(1H)-one (23-1), and (3aR,6aR)-1,3-bis(4-chlorophenyl)-3a-(3-(difluoromethoxy)phenyl)hexahydro cyclopenta[d]imidazol-2(1H)-one (23-2)

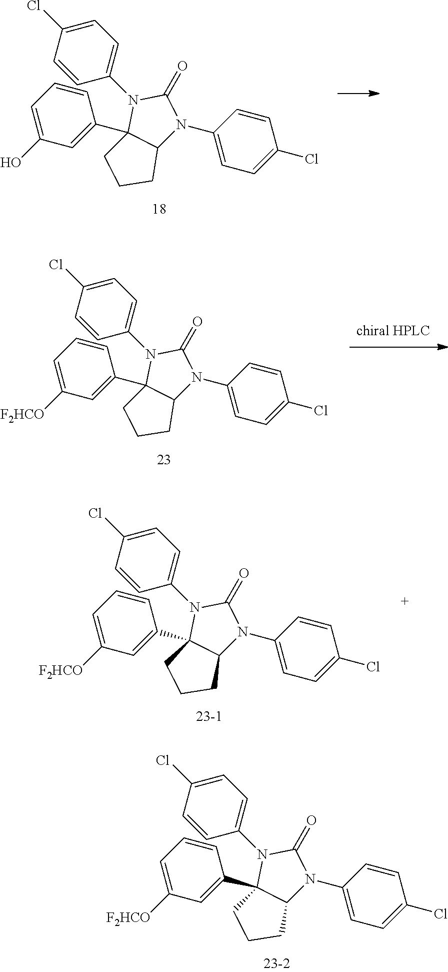 [SMILES: O=C1N(c2ccc(Cl)cc2)C2CCCC2(c2cccc(O)c2)N1c1ccc(Cl)cc1 ; O=C1N(c2ccc(Cl)cc2)C2CCCC2(c2cccc(OC(F)F)c2)N1c1ccc(Cl)cc1 ; O=C1N(c2ccc(Cl)cc2)[C@H]2CCC[C@@]2(c2cccc(OC(F)F)c2)N1c1ccc(Cl)cc1 ; O=C1N(c2ccc(Cl)cc2)[C@@H]2CCC[C@]2(c2cccc(OC(F)F)c2)N1c1ccc(Cl)cc1]

To a solution of Compound 18 (100 mg, 0.227 mmol) in acetonitrile (5 mL) and KOH solution (30%, 5 mL) was added 2-chloro-2,2-difluoro-1-phenylethanone (870 mg, 2.27 mmol). The mixture was stirred at 80° C. for 8 hours, poured into a saturated sodium bicarbonate solution (50 mL), and extracted with ethyl acetate (50 mL×3). The combined extracts were washed with brine (100 mL), dried over anhydrous sodium sulfate, filtered, and concentrated to give a crude product, which was purified with preparative HPLC to afford Compound 23, which was separated with chiral HPLC to afford Compound 23-1 and Compound 23-2.

Compound 23-1: LC-MS (ESI) m/z: 489 [M+H]⁺; ¹H-NMR (CDCl₃, 400 MHz): δ (ppm) 1.91-2.04 (m, 3H), 2.11-2.19 (m, 1H), 2.27-2.43 (m, 2H), 4.37-4.39 (m, 1H), 6.32-6.69 (m, 1H), 6.98-7.02 (m, 2H), 7.10-7.13 (m, 2H), 7.18-7.22 (m, 2H), 7.25-7.27 (m, 1H), 7.30-7.33 (m, 2H), 7.37-7.41 (m, 1H), 7.56-7.60 (m, 2H). Chiral separation conditions: MeOH contained 0.2% methanol ammonia; OJ (4.6×250 mm, 5 μm); retention time: 1.5 minutes.

Compound 23-2: LC-MS (ESI) m/z: 489 [M+H]⁺; ¹H-NMR (CDCl₃, 400 MHz): δ (ppm) 1.92-2.06 (m, 3H), 2.11-2.19 (m, 1H), 2.26-2.42 (m, 2H), 4.37-4.39 (m, 1H), 6.32-6.69 (m, 1H), 6.98-7.02 (m, 2H), 7.10-7.13 (m, 2H), 7.18-7.22 (m, 2H), 7.25-7.27 (m, 1H), 7.30-7.33 (m, 2H), 7.37-7.41 (m, 1H), 7.56-7.60 (m, 2H). Chiral separation conditions: MeOH contained 0.2% methanol ammonia; OJ (4.6×250 mm, 5 μm); retention time: 2.1 minutes.

### Example 24

### Synthesis of 3-(5,5-dimethyl-2-oxo-1,3-bis(4-(trifluoromethyl)phenyl) imidazolidin-4-yl)benzonitrile (24), (R)-3-(5,5-dimethyl-2-oxo-1,3-bis(4-(trifluoromethyl) phenyl)imidazolidin-4-yl)benzonitrile (24-1), and (S)-3-(5,5-dimethyl-2-oxo-1,3-bis(4-(trifluoromethyl)phenyl)imidazolidin-4-yl)benzonitrile (24-2)

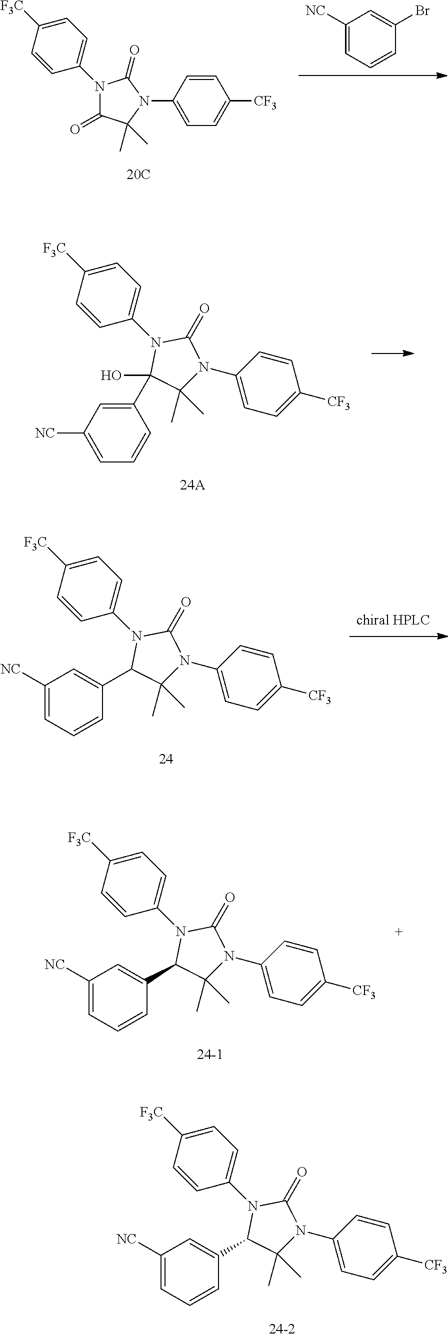 [SMILES: CC1(C)C(=O)N(c2ccc(C(F)(F)F)cc2)C(=O)N1c1ccc(C(F)(F)F)cc1 ; N#Cc1cccc(Br)c1 ; CC1(C)N(c2ccc(C(F)(F)F)cc2)C(=O)N(c2ccc(C(F)(F)F)cc2)C1(O)c1cccc(C#N)c1 ; CC1(C)C(c2cccc(C#N)c2)N(c2ccc(C(F)(F)F)cc2)C(=O)N1c1ccc(C(F)(F)F)cc1 ; CC1(C)[C@@H](c2cccc(C#N)c2)N(c2ccc(C(F)(F)F)cc2)C(=O)N1c1ccc(C(F)(F)F)cc1 ; CC1(C)[C@H](c2cccc(C#N)c2)N(c2ccc(C(F)(F)F)cc2)C(=O)N1c1ccc(C(F)(F)F)cc1]

Compounds 24A and 24 were synthesized by employing the procedures described for Compounds 6D and 6 using 3-bromobenzonitrile, Compounds 20C, and 24A at room temperature in lieu of 1-bromo-3-(trifluoromethyl)benzene, Compounds 6C, and 6D at 10° C.

Compound 24A. LC-MS (ESI) m/z: 520 [M+H]⁺. ¹H-NMR (DMSO-d₆, 400 MHz): δ (ppm) 0.83 (s, 3H), 1.31 (s, 3H), 7.53 (s, 1H), 7.61-7.73 (m, 7H), 7.79-7.88 (m, 5H).

Compound 24 was separated with chiral HPLC to afford Compound 24-1 and Compound 24-2.

Compound 24-1: LC-MS (ESI) m/z: 504 [M+H]⁺; ¹H-NMR (CD₃OD, 400 MHz): δ (ppm) 0.86 (s, 3H), 1.61 (s, 3H), 5.49 (s, 1H), 7.52-7.56 (m, 5H), 7.70-7.79 (m, 7H). Chiral separation conditions: MeOH contained 0.2% Methanol Ammonia; R,R-Whelk-01 (4.6×100 mm, 5 μm); retention time: 2.55 minutes.

Compound 24-2: LC-MS (ESI) m/z: 504 [M+H]⁺; ¹H-NMR (CD₃OD, 400 MHz): δ (ppm) 0.75 (s, 3H), 1.50 (s, 3H), 5.38 (s, 1H), 7.40-7.45 (m, 5H), 7.57-7.68 (m, 7H). Chiral separation conditions: MeOH contained 0.2% Methanol Ammonia; R,R-Whelk-01 (4.6×100 mm, 5 μm); retention time: 4.72 min.

### Example 25

### Synthesis of (4S,5S)-1,3-bis(4-chlorophenyl)-4-methyl-5-(3-(trifluoromethoxy)phenyl)-4-(trifluoromethyl)imidazolidin-2-one (25-1) and (4R,5R)-1,3-bis(4-chlorophenyl)-4-methyl-5-(3-(trifluoromethoxy)phenyl)-4-(trifluoromethyl) imidazolidin-2-one (25-2)

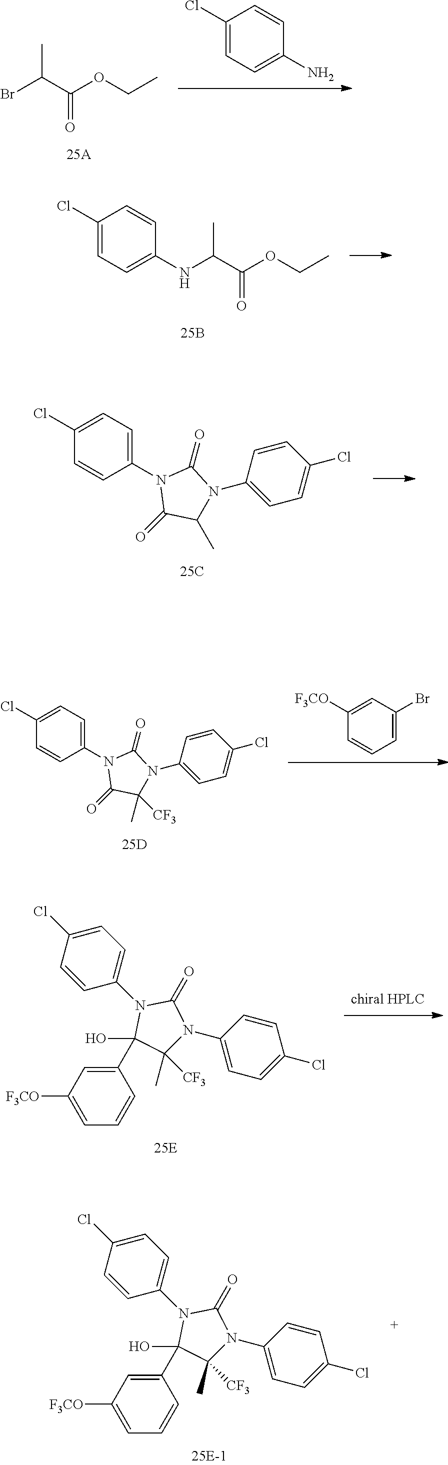

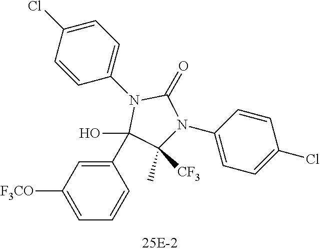 [SMILES: C[C@@]1(C(F)(F)F)N(c2ccc(Cl)cc2)C(=O)N(c2ccc(Cl)cc2)C1(O)c1cccc(OC(F)(F)F)c1]

A mixture of ethyl 2-bromopropanoate 25A (3.81 g, 21 mmol), 4-chloroaniline (2.67 g, 21 mmol), and sodium acetate (3.4 g, 42 mmol) was stirred at 125° C. overnight. After cooling down to room temperature, the mixture was diluted with water (50 mL) and extracted with ethyl acetate (100 mL×2). The combined extracts were washed with brine (100 mL), dried over anhydrous sodium sulfate, filtered, and concentrated to give a crude product, which was purified with flash column chromatography on silica gel (ethyl acetate in petroleum ether, from 0% to 8% v/v) to yield Compound 25B. LC-MS (ESI) m/z: 228 [M+H]⁺; ¹H-NMR (CDCl₃, 400 MHz): δ (ppm) 1.25 (d, J=6.8 Hz, 3H), 1.46 (d, J=6.8 Hz, 3H), 4.04-4.11 (m, 1H), 4.14-4.22 (m, 3H), 6.53 (d, J=8.8 Hz, 2H), 7.12 (d, J=8.8 Hz, 2H).

A mixture of Compound 25B (3.0 g, 13.2 mmol) and 1-chloro-4-isocyanatobenzene (2.0 g, 13.2 mmol) in pyridine (5 mL) was stirred at 50° C. for 16 hours and concentrated. The residue was purified with flash column chromatography on silica gel (ethyl acetate in petroleum ether, 10% v/v) to afford Compound 25C. LC-MS (ESI) m/z: 335 [M+H]⁺; ¹H-NMR (CDCl₃, 400 MHz): δ (ppm) 1.58 (d, J=7.2 Hz, 3H), 4.68-4.73 (m, 1H), 7.37-7.48 (m, 8H).

To a solution of Compound 25C (1.5 g, 5.0 mmol) in anhydrous THF (15 mL) was dropped a solution of LDA in n-hexane (1.0 M, 5.5 mL, 5.5 mmol) at −78° C. under nitrogen atmosphere and the mixture was stirred at −78° C. for 30 minutes. To the mixture was added 5-(trifluoromethyl)-5H-dibenzo[b,d]thiophenium trifluoromethanesulfonate (2.2 g, 5.5 mmol) in one portion, stirred at −78° C. for 10 minutes, and the mixture was stirred at room temperature overnight. The mixture was quenched with saturated ammonium chloride solution (20 mL) and extracted with ethyl acetate (30 ml×2). The combined extracts were washed with brine (40 mL), dried over anhydrous sodium sulfate, filtered, and evaporated under reduced pressure. The residue was purified with preparative HPLC to furnish Compound 25D. LC-MS (ESI) m/z: 403 [M+H]⁺; ¹H-NMR (CDCl₃, 400 MHz): δ (ppm) 1.81 (s, 3H), 7.25-7.27 (m, 2H), 7.41-7.48 (m, 6H).

Compound 25E was synthesized by employing the procedure described for Compound 6D using 1-bromo-3-(trifluoromethoxy)benzene and Compounds 25D in lieu of 1-bromo-3-(trifluoromethyl)benzene and Compound 6C. LC-MS (ESI) m/z: 565 [M+H]⁺; ¹H-NMR (CDCl₃, 400 MHz): δ (ppm) 1.03 (s, 3H), 7.185-7.22 (m, 4H), 7.26-7.31 (m, 4H), 7.39-7.42 (m, 4H). Compound 25E was separated with chiral HPLC to afford Compound 25E-1 and Compound 25E-2.

Compound 25E-1: LC-MS (ESI) m/z: 565 [M+H]⁺; ¹H-NMR (CDCl₃, 400 MHz): δ (ppm) 1.04 (s, 3H), 3.49 (s, 1H), 7.19-7.31 (m, 8H), 7.40-7.43 (m, 4H). Chiral separation conditions: MeOH contained 0.2% Methanol Ammonia; OJ-H (4.6×100 mm, 5 μm); retention time: 0.83 minutes.

Compound 25E-2: LC-MS (ESI) m/z: 565 [M+H]⁺; ¹H-NMR (CDCl₃, 400 MHz): δ (ppm) 1.04 (s, 3H), 3.47 (s, 1H), 7.14-7.31 (m, 8H), 7.40-7.52 (m, 4H). Chiral separation conditions: MeOH contained 0.2% Methanol Ammonia; OJ-H (4.6×100 mm, 5 μm); retention time: 1.01 minutes.

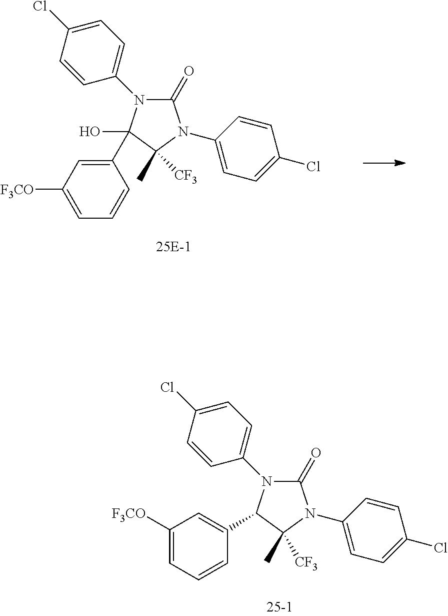 [SMILES: C[C@]1(C(F)(F)F)N(c2ccc(Cl)cc2)C(=O)N(c2ccc(Cl)cc2)C1(O)c1cccc(OC(F)(F)F)c1 ; C[C@@]1(C(F)(F)F)[C@H](c2cccc(OC(F)(F)F)c2)N(c2ccc(Cl)cc2)C(=O)N1c1ccc(Cl)cc1]

Compound 25-1 was synthesized by employing the procedure described for Compound 6 using Compound 25E-1 at room temperature in lieu of Compound 6D at 10° C. LC-MS (ESI) m/z: 549 [M+H]⁺; ¹H-NMR (CDCl₃, 400 MHz): δ (ppm) 1.79 (s, 3H), 5.29 (s, 1H), 7.20-7.26 (m, 8H), 7.41-7.44 (m, 4H); ¹H-NMR-NOESY (CDCl₃, 400 MHz): Me (1.79 ppm) has correlation with proton H (5.29 ppm). Chiral separation conditions: MeOH contained 0.2% Methanol Ammonia; Cellulose-SC (4.6×100 mm, 5 μm); retention time: 0.97 minutes.

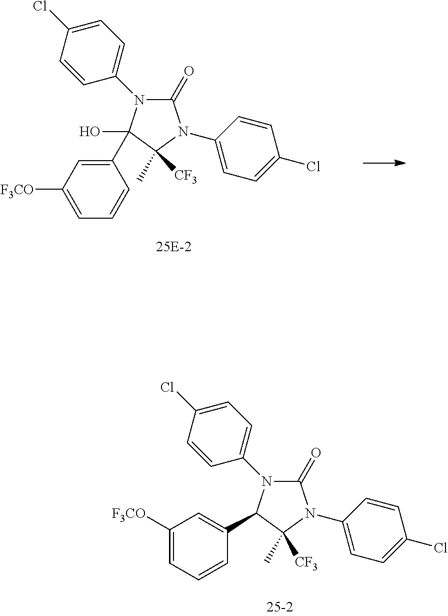 [SMILES: C[C@@]1(C(F)(F)F)N(c2ccc(Cl)cc2)C(=O)N(c2ccc(Cl)cc2)C1(O)c1cccc(OC(F)(F)F)c1 ; C[C@]1(C(F)(F)F)[C@@H](c2cccc(OC(F)(F)F)c2)N(c2ccc(Cl)cc2)C(=O)N1c1ccc(Cl)cc1]

Compound 25-2 was synthesized by employing the procedure described for Compound 6 using Compound 25E-2 at room temperature in lieu of Compound 6D at 10° C. LC-MS (ESI) m/z: 549 [M+H]⁺; ¹H-NMR (CDCl₃, 400 MHz): δ (ppm) 1.79 (s, 3H), 5.31 (s, 1H), 7.20-7.26 (m, 8H), 7.41-7.44 (m, 4H); ¹H-NMR-NOESY (CDCl₃, 400 MHz): Me (1.79 ppm) has a correlation with proton H (5.31 ppm). Chiral separation conditions: MeOH contained 0.2% Methanol Ammonia; Cellulose-SC (4.6×100 mm, 5 μm); retention time: 0.59 minutes.

### Example 26

### Synthesis of 4,4-dimethyl-1,3-diphenyl-5-(3-(trifluoromethoxy)phenyl) imidazolidin-2-one (26), (R)-4,4-dimethyl-1,3-diphenyl-5-(3-(trifluoromethoxy)phenyl) imidazolidin-2-one (26-1), and (S)-4,4-dimethyl-1,3-diphenyl-5-(3-(trifluoromethoxy) phenyl)imidazolidin-2-one (26-2)

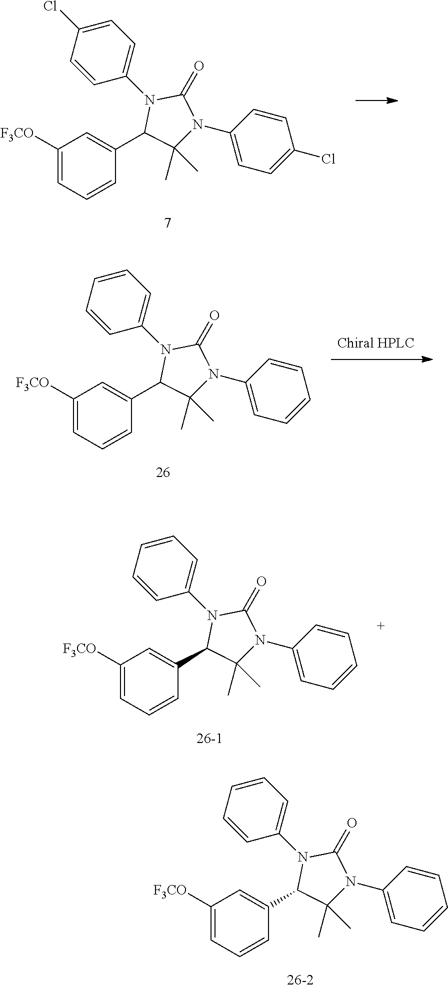 [SMILES: CC1(C)C(c2cccc(OC(F)(F)F)c2)N(c2ccc(Cl)cc2)C(=O)N1c1ccc(Cl)cc1 ; CC1(C)C(c2cccc(OC(F)(F)F)c2)N(c2ccccc2)C(=O)N1c1ccccc1 ; CC1(C)[C@@H](c2cccc(OC(F)(F)F)c2)N(c2ccccc2)C(=O)N1c1ccccc1 ; CC1(C)[C@H](c2cccc(OC(F)(F)F)c2)N(c2ccccc2)C(=O)N1c1ccccc1]

A mixture of Compound 7 (50 mg, 0.101 mmol) and palladium on activated carbon catalyst (5 mg, 10% w/w) in methanol (5 mL) was stirred at room temperature under hydrogen atmosphere (1 atm.) for 18 hours. The reaction mixture was filtered through Celite. The filtrate was concentrated and purified with preparative HPLC to afford Compound 26, which was separated with chiral HPLC to furnish Compound 26-1 and Compound 26-2.

Compound 26-1: LC-MS (ESI) m/z: 427 [M+H]⁺; ¹H-NMR (CDCl₃, 400 MHz): δ (ppm) 0.80 (s, 3H), 1.53 (s, 3H), 5.02 (s, 1H), 6.97-7.04 (m, 1H), 7.15-7.27 (m, 7H), 7.32-7.48 (m, 6H). Chiral separation conditions: MeOH contained 0.2% methanol ammonia; OD (4.6*250 mm, 5 μm); retention time: 1.4 minutes.

Compound 26-2: LC-MS (ESI) m/z: 427 [M+H]⁺; ¹H-NMR (CDCl₃, 400 MHz): δ (ppm) 0.80 (s, 3H), 1.53 (s, 3H), 5.02 (s, 1H), 6.97-7.04 (m, 1H), 7.17-7.28 (m, 7H), 7.34-7.46 (m, 6H). Chiral separation conditions: MeOH contained 0.2% methanol ammonia; OD (4.6*250 mm, 5 μm); retention time: 3.03 minutes.

### Example 27

### Synthesis of 3-((4S,5S)-1,3-bis(4-chlorophenyl)-5-methyl-2-oxo-5-(trifluoromethyl)imidazolidin-4-yl)benzonitrile (27-1) and 3-((4R,5R)-1,3-bis(4-chlorophenyl)-5-methyl-2-oxo-5-(trifluoromethyl)imidazolidin-4-yl)benzonitrile (27-2)

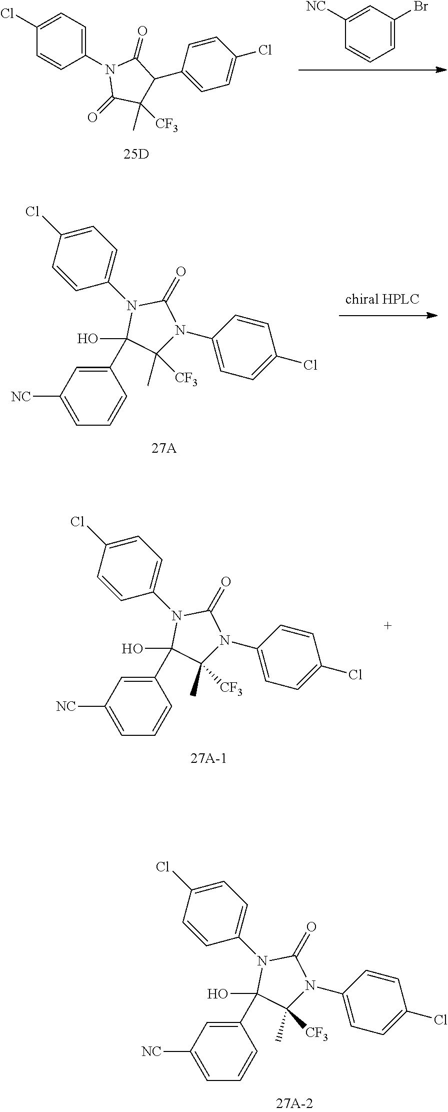 [SMILES: CC1(C(F)(F)F)C(=O)N(c2ccc(Cl)cc2)C(=O)C1c1ccc(Cl)cc1 ; N#Cc1cccc(Br)c1 ; CC1(C(F)(F)F)N(c2ccc(Cl)cc2)C(=O)N(c2ccc(Cl)cc2)C1(O)c1cccc(C#N)c1 ; C[C@]1(C(F)(F)F)N(c2ccc(Cl)cc2)C(=O)N(c2ccc(Cl)cc2)C1(O)c1cccc(C#N)c1 ; C[C@@]1(C(F)(F)F)N(c2ccc(Cl)cc2)C(=O)N(c2ccc(Cl)cc2)C1(O)c1cccc(C#N)c1]

Compound 27A was synthesized by employing the procedure described for Compound 6D using 3-bromobenzonitrile and Compounds 25D in lieu of 1-bromo-3-(trifluoromethyl)benzene and Compound 6C. LC-MS (ESI) m/z: 506 [M+H]⁺; ¹H-NMR (CDCl₃, 400 MHz): δ (ppm) 1.05 (s, 3H), 3.75 (s, 1H), 7.19 (d, J=8.8 Hz, 4H), 7.26-7.29 (m, 2H), 7.30 (d, J=8.8 Hz, 2H), 7.42 (d, J=8.8 Hz, 2H), 7.50-7.55 (m, 1H), 7.70 (d, J=7.2 Hz, 1H). Compound 27A was separated with chiral HPLC to afford Compound 27A-1 and Compound 27A-2.

Compound 27A-1: LC-MS (ESI) m/z: 506 [M+H]⁺; ¹H-NMR (CDCl₃, 400 MHz): δ (ppm) 1.05 (s, 3H), 3.93 (s, 1H), 7.18-7.22 (m, 4H), 7.26-7.31 (m, 3H), 7.41-7.43 (d, J=8.8 Hz, 2H), 7.43-7.50 (m, 1H), 7.70 (d, J=7.2 Hz, 2H). Chiral separation conditions: MeOH contained 0.2% Methanol Ammonia; (R, R)-Whelk-Ol (4.6×250 mm, 5 μm); retention time: 2.22 minutes.

Compound 27A-2: LC-MS (ESI) m/z: 506 [M+H]⁺; ¹H-NMR (CDCl₃, 400 MHz): δ (ppm) 1.05 (s, 3H), 4.04 (s, 1H), 7.18-7.22 (m, 4H), 7.26-7.31 (m, 3H), 7.40-7.43 (d, J=8.8 Hz, 2H), 7.43-7.52 (m, 1H), 7.67 (d, J=7.2 Hz, 2H), Chiral separation conditions: MeOH contained 0.2% Methanol Ammonia; (R, R)-Whelk-Ol (4.6×250 mm, 5 μm); retention time: 3.66 minutes.

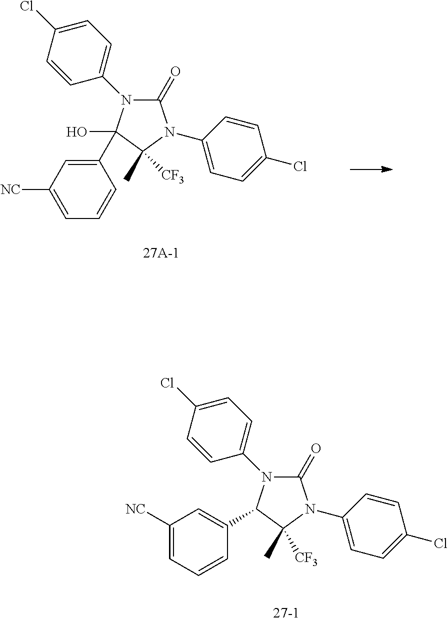 [SMILES: C[C@]1(C(F)(F)F)N(c2ccc(Cl)cc2)C(=O)N(c2ccc(Cl)cc2)C1(O)c1cccc(C#N)c1 ; C[C@@]1(C(F)(F)F)[C@H](c2cccc(C#N)c2)N(c2ccc(Cl)cc2)C(=O)N1c1ccc(Cl)cc1]

Compound 27-1 was synthesized by employing the procedure described for Compound 6 using Compound 27A-1 at room temperature in lieu of Compound 6D at 10° C. LC-MS (ESI) m/z: 490 [M+H]⁺; ¹H-NMR (CDCl₃, 400 MHz): δ (ppm) 1.80 (s, 3H), 5.31 (s, 1H), 7.14-7.26 (m, 6H), 7.41-7.72 (m, 6H). ¹H-NMR-NOESY (CDCl₃, 400 MHz): Me (1.80 ppm) has correlation with proton H (5.31 ppm). Chiral separation conditions: MeOH contained 0.2% Methanol Ammonia; OJ-H (4.6×250 mm, 5 μm); retention time: 3.56 minutes.

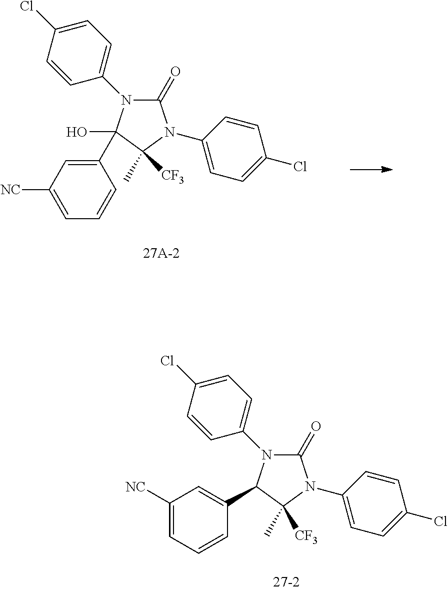 [SMILES: C[C@@]1(C(F)(F)F)N(c2ccc(Cl)cc2)C(=O)N(c2ccc(Cl)cc2)C1(O)c1cccc(C#N)c1 ; C[C@]1(C(F)(F)F)[C@@H](c2cccc(C#N)c2)N(c2ccc(Cl)cc2)C(=O)N1c1ccc(Cl)cc1]

Compound 27-2 was synthesized by employing the procedure described for Compound 6 using Compound 27A-2 at room temperature in lieu of Compound 6D at 10° C. LC-MS (ESI) m/z: 490 [M+H]⁺; ¹H-NMR (CDCl₃, 400 MHz): δ (ppm) 1.81 (s, 3H), 5.31 (s, 1H), 7.14-7.26 (m, 6H), 7.42-7.69 (m, 6H). ¹H-NMR-NOESY (CDCl₃, 400 MHz): Me (1.81 ppm) has a correlation with proton H (5.31 ppm). Chiral separation conditions: MeOH contained 0.2% Methanol Ammonia; OJ-H (4.6×250 mm, 5 μm); retention time: 4.07 minutes.

### Example 28

### Synthesis of 1,3-bis(3-chlorophenyl)-4,4-dimethyl-5-(3-(trifluoromethoxy)phenyl)imidazolidin-2-one (28), (R)-1,3-bis(3-chlorophenyl)-4,4-dimethyl-5-(3-(trifluoromethyl)phenyl)imidazolidin-2-one (28-1), and (S)-1,3-bis(3-chlorophenyl)-4,4-dimethyl-5-(3-(trifluoromethyl)phenyl)imidazolidin-2-one (28-2)

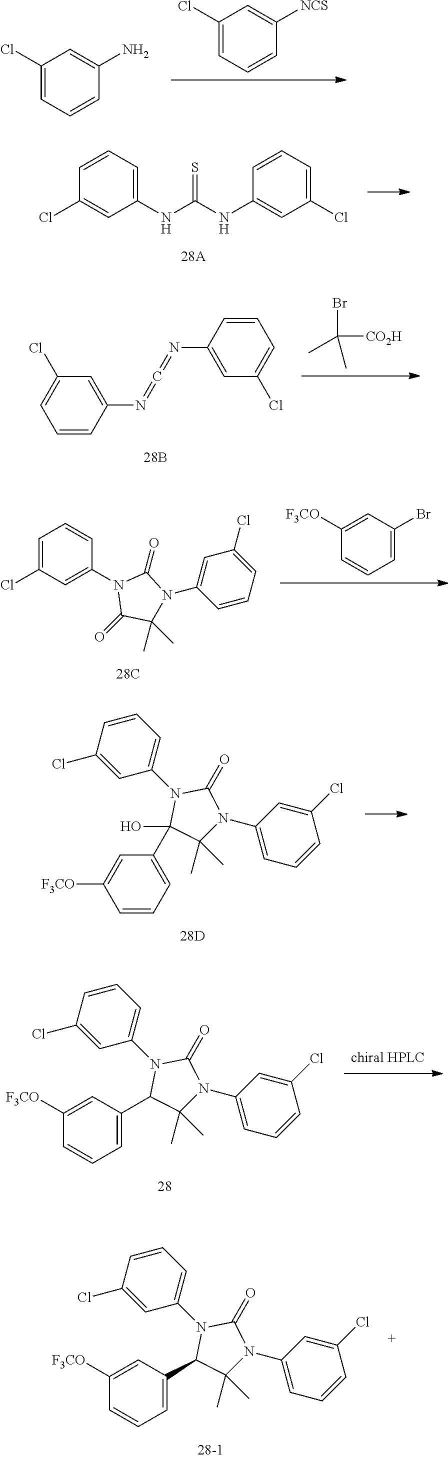

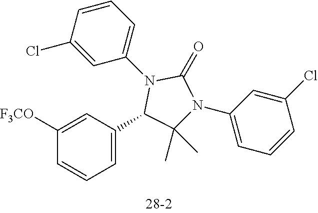 [SMILES: CC1(C)[C@H](c2cccc(OC(F)(F)F)c2)N(c2cccc(Cl)c2)C(=O)N1c1cccc(Cl)c1]

Compounds 28A, 28B, 28C, 28D, and 28 were synthesized by employing the procedures described for Compounds 6A, 6B, 6C, 6D, and 6 using 1-chloro-3-isothiocyanatobenzene, 3-chloroaniline, Compounds 28A, 28B, 28C, 1-bromo-3-(trifluoromethoxy)benzene, and 28D at room temperature in lieu of 1-chloro-4-isothiocyanatobenzene, 4-chloroaniline, Compounds 6A, 6B, 6C, 1-bromo-3-(trifluoromethyl)benzene, and 6D at 10° C.

Compound 28A. LC-MS (ESI) m/z: 297 [M+H]⁺.

Compound 28B. LC-MS: non-ionizable under routine conditions.

Compound 28C. LC-MS (ESI) m/z: 349 [M+H]⁺.

Compound 28D. LC-MS (ESI) m/z: 511 [M+H]⁺. ¹H-NMR (400 MHz, CDCl₃): δ (ppm) 0.73 (s, 3H), 1.24 (s, 3H), 5.06 (s, 1H), 6.96-7.07 (m, 3H), 7.18-7.27 (m, 8H), 7.49-7.50 (m, 1H).

Compound 28 was separated with chiral HPLC to yield Compound 28-1 and Compound 28-2.

Compound 28-1: LC-MS (ESI) m/z: 495 [M+H]⁺; ¹H-NMR (CDCl₃, 400 MHz): δ (ppm) 0.81 (s, 3H), 1.54 (s, 3H), 4.96 (s, 1H), 6.97-6.99 (m, 1H), 7.12-7.25 (m, 7H), 7.33-7.43 (m, 3H), 7.59 (s, 1H). Chiral separation conditions: MeOH contained 0.2% methanol ammonia; Enantiopak OD (4.6×100 mm, 5 um); retention time: 1.89 minutes.

Compound 28-2: LC-MS (ESI) m/z: 495 [M+H]⁺; ¹H-NMR (CDCl₃, 400 MHz): δ (ppm) 0.81 (s, 3H), 1.55 (s, 3H), 4.95 (s, 1H), 6.97-6.99 (m, 1H), 7.12-7.25 (m, 7H), 7.33-7.43 (m, 3H), 7.58 (s, 1H). Chiral separation conditions: MeOH contained 0.2% methanol ammonia; Enantiopak OD (4.6×100 mm, 5 um); retention time: 0.94 minutes.

### Example 29

### Synthesis of 5,7-bis(4-chlorophenyl)-8-(3-(trifluoromethoxy)phenyl)-2-oxa-5,7-diazaspiro[3.4]octan-6-one (29), (R)-5,7-bis(4-chlorophenyl)-8-(3-(trifluoromethoxy) phenyl)-2-oxa-5,7-diazaspiro[3.4]octan-6-one (29-1), and (S)-5,7-bis(4-chlorophenyl)-8-(3-(trifluoromethoxy)phenyl)-2-oxa-5,7-diazaspiro[3.4]octan-6-one (29-2)

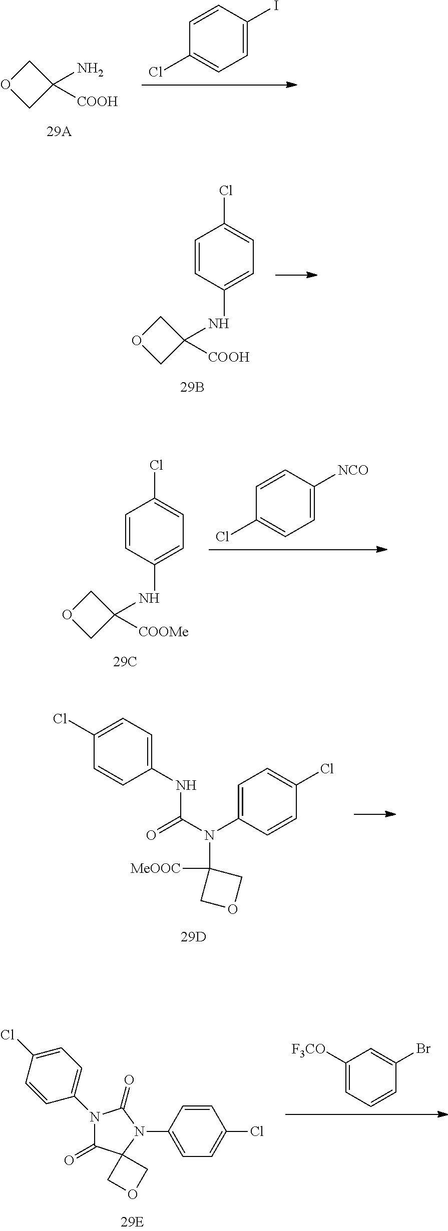

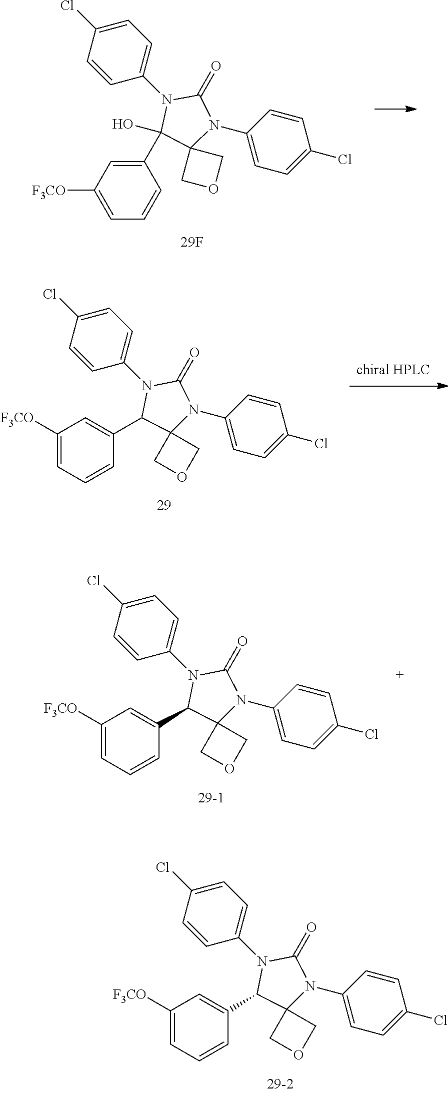 [SMILES: O=C1N(c2ccc(Cl)cc2)C2(COC2)C(O)(c2cccc(OC(F)(F)F)c2)N1c1ccc(Cl)cc1 ; O=C1N(c2ccc(Cl)cc2)C(c2cccc(OC(F)(F)F)c2)C2(COC2)N1c1ccc(Cl)cc1 ; O=C1N(c2ccc(Cl)cc2)[C@H](c2cccc(OC(F)(F)F)c2)C2(COC2)N1c1ccc(Cl)cc1 ; O=C1N(c2ccc(Cl)cc2)[C@@H](c2cccc(OC(F)(F)F)c2)C2(COC2)N1c1ccc(Cl)cc1]

Compound 29B was synthesized by employing the procedure described for Compound 12B using Compound 29A in lieu of Compound 12A, which was used directly in the next step without further purification. LC-MS (ESI) m/z: 228 [M+H]⁺.

To a solution of Compound 29B (800 mg, 3.51 mmol) in dichloromethane (20 mL) and methanol (5 mL) was added a solution of TMSCHN₂ in n-hexane (2 M, 2.6 mL, 5.27 mmol). The mixture was stirred at room temperature overnight and concentrated under reduced pressure. The residue was purified with flash column chromatography on silica gel (ethyl acetate in petroleum ether, from 0% to 15% v/v) to yield Compound 29C. LC-MS (ESI) m/z: 242 [M+H]⁺.

A mixture of Compound 29C (700 mg, 2.9 mmol) and 1-chloro-4-isocyanatobenzene (467 mg, 3.04 mmol) in pyridine (10 mL) was stirred at 80° C. overnight and concentrated under reduced pressure. The residue was purified with flash column chromatography on silica gel to yield Compound 29D. LC-MS (ESI) m/z: 395 [M+H]⁺.

Compounds 29E and 29F were synthesized by employing the procedures described for Compounds 12E and 6D using Compound 29D using Et₃N as base, 29E, and 1-bromo-3-(trifluoromethoxy)benzene in lieu of Compounds 12D using NH₃ as base, 6C, and 1-bromo-3-(trifluoromethyl)benzene.

Compound 29E. LC-MS (ESI) m/z: 363 [M+H]⁺; ¹H-NMR (CDCl₃, 400 MHz): δ (ppm) 4.91 (d, J=7.6 Hz, 2H), 5.15 (d, J=7.6 Hz, 2H), 7.45-7.55 (m, 8H).

Compound 29F. LC-MS (ESI) m/z: 525 [M+H]⁺; ¹H-NMR (CDCl₃, 400 MHz): δ (ppm) 3.96 (d, J=8.0 Hz, 1H), 4.38 (d, J=8.0 Hz, 1H), 4.57 (d, J=8.4 Hz, 1H), 5.01 (s, 1H), 5.12 (d, J=8.8 Hz, 1H), 7.11-7.22 (m, 7H), 7.30-7.42 (m, 5H).

To a stirred solution of Compound 29F (70 mg, 0.1336 mmol) in dichloromethane (5 mL) was added Et₃SiH (78 mg, 0.668 mmol) and TFA (76 mg, 0.668 mmol). The mixture was stirred at room temperature for three days and concentrated under reduced pressure. The residue was purified with preparative HPLC to furnish Compound 29, which was separated with chiral HPLC to yield Compound 29-1 and Compound 29-2.

Compound 29-1: LC-MS (ESI) m/z: 509 [M+H]⁺; ¹H-NMR (CDCl₃, 400 MHz): δ (ppm) 4.17 (d, J=6.8 Hz, 1H), 4.52 (d, J=6.4 Hz, 1H), 4.84 (d, J=6.0 Hz, 1H), 5.04 (d, J=6.0 Hz, 1H), 5.46 (s, 1H), 7.23-7.53 (m, 12H). Chiral separation conditions: MeOH contained 0.2% Methanol Ammonia; EnantioPak OD (4.6×100 mm, 5 μm); retention time: 1.33 minutes.

Compound 29-2: LC-MS (ESI) m/z: 509 [M+H]⁺; ¹H-NMR (CDCl₃, 400 MHz): δ (ppm) 4.17 (d, J=6.8 Hz, 1H), 4.52 (d, J=6.4 Hz, 1H), 4.84 (d, J=6.0 Hz, 1H), 5.04 (d, J=6.0 Hz, 1H), 5.46 (s, 1H), 7.23-7.53 (m, 12H); Chiral separation conditions: MeOH contained 0.2% Methanol Ammonia; EnantioPak OD (4.6×100 mm, 5 μm); retention time: 3.09 minutes.

### Example 30

### Synthesis of 3-(5,7-bis(4-chlorophenyl)-6-oxo-2-oxa-5,7-diazaspiro[3.4]octan-8-yl)benzonitrile (30), (R)-3-(5,7-bis(4-chlorophenyl)-6-oxo-2-oxa-5,7-diazaspiro[3.4]octan-8-yl)benzonitrile (30-1), and (S)-3-(5,7-bis(4-chlorophenyl)-6-oxo-2-oxa-5,7-diazaspiro[3.4]octan-8-yl)benzonitrile (30-2)

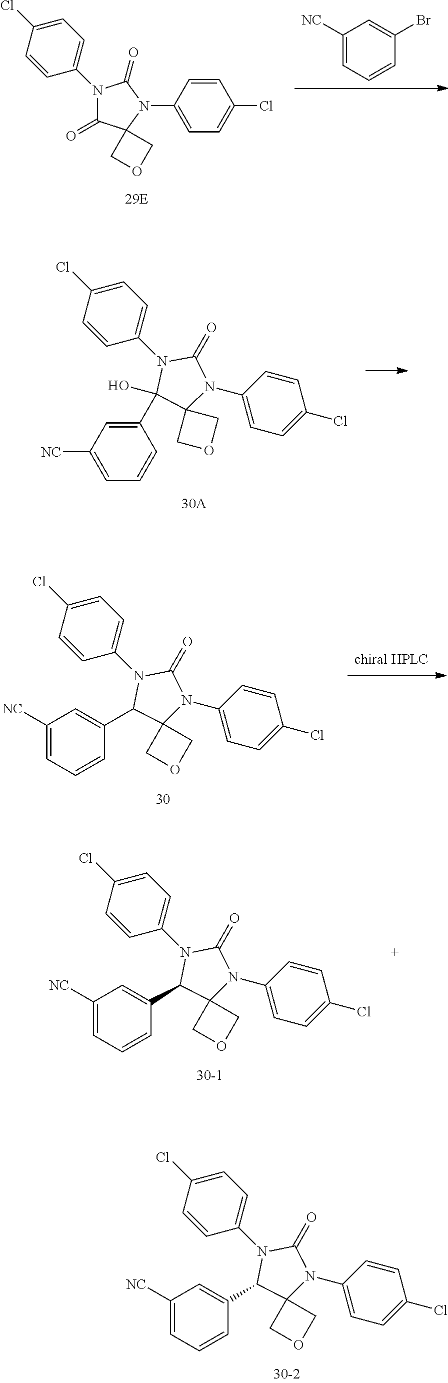 [SMILES: O=C1N(c2ccc(Cl)cc2)C(=O)C2(COC2)N1c1ccc(Cl)cc1 ; N#Cc1cccc(Br)c1 ; N#Cc1cccc(C2(O)N(c3ccc(Cl)cc3)C(=O)N(c3ccc(Cl)cc3)C23COC3)c1 ; N#Cc1cccc(C2N(c3ccc(Cl)cc3)C(=O)N(c3ccc(Cl)cc3)C23COC3)c1 ; N#Cc1cccc([C@H]2N(c3ccc(Cl)cc3)C(=O)N(c3ccc(Cl)cc3)C23COC3)c1 ; N#Cc1cccc([C@@H]2N(c3ccc(Cl)cc3)C(=O)N(c3ccc(Cl)cc3)C23COC3)c1]

Compounds 30A and 30 were synthesized by employing the procedures described for Compounds 6D and 29 using 3-bromobenzonitrile, Compounds 29E, and 30A in lieu of 1-bromo-3-(trifluoromethyl)benzene, Compounds 6C, and 29F.

Compound 30A. LC-MS (ESI) m/z: 466 [M+H]⁺; ¹H-NMR (CDCl₃, 400 MHz): δ (ppm) 3.87 (d, J=7.6 Hz, 1H), 4.41 (d, J=8.4 Hz, 1H), 4.63 (d, J=8.4 Hz, 1H), 5.05 (s, 1H), 5.12 (d, J=8.4 Hz, 1H), 7.14-7.20 (m, 6H), 7.37 (d, J=8.8 Hz, 2H), 7.45-7.55 (m, 2H), 7.64 (d, J=8.4 Hz, 1H), 7.84 (s, 1H).

Compound 30 was separated with chiral HPLC to yield Compound 30-1 and Compound 30-2.

Compound 30-1: LC-MS (ESI) m/z: 450 [M+H]⁺; ¹H-NMR (CDCl₃, 400 MHz): δ (ppm) 4.08 (d, J=8.4 Hz, 1H), 4.55 (d, J=8.0 Hz, 1H), 4.83 (d, J=7.6 Hz, 1H), 5.05 (d, J=7.2 Hz, 1H), 5.51 (s, 1H), 7.23-7.40 (m, 6H), 7.51-7.73 (m, 6H). Chiral separation conditions: MeOH contained 0.2% Methanol Ammonia; Cellulose-SC (4.6×100 mm, 5 μm); retention time: 1.3 minutes.

Compound 30-2: LC-MS (ESI) m/z: 450 [M+H]⁺; ¹H-NMR (CDCl₃, 400 MHz): δ (ppm) 4.08 (d, J=8.4 Hz, 1H), 4.55 (d, J=8.0 Hz, 1H), 4.83 (d, J=7.2 Hz, 1H), 5.05 (d, J=7.6 Hz, 1H), 5.51 (s, 1H), 7.23-7.40 (m, 6H), 7.51-7.73 (m, 6H). Chiral separation conditions: MeOH contained 0.2% Methanol Ammonia; Cellulose-SC (4.6×100 mm, 5 μm); retention time: 1.74 minutes.

### Example 31

### Synthesis of 1,3-bis(4-chlorophenyl)-3a-(3-(trifluoromethoxy)phenyl) hexahydropyrano[3,4-d]imidazol-2(3H)-one (31), (3aR)-1,3-bis(4-chlorophenyl)-3a-(4-(trifluoromethoxy)phenyl)hexahydropyrano[3,4-d]imidazol-2(3H)-one (31-1), and (3 aS)-1,3-bis(4-chlorophenyl)-3a-(4-(trifluoromethoxy)phenyl)hexahydropyrano[3,4-d]imidazol-2(3H)-one (31-2)

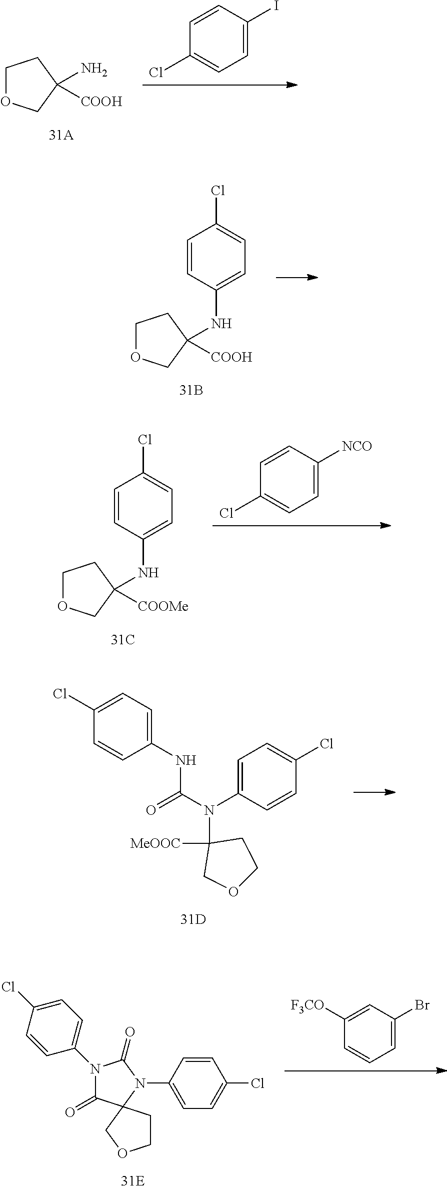

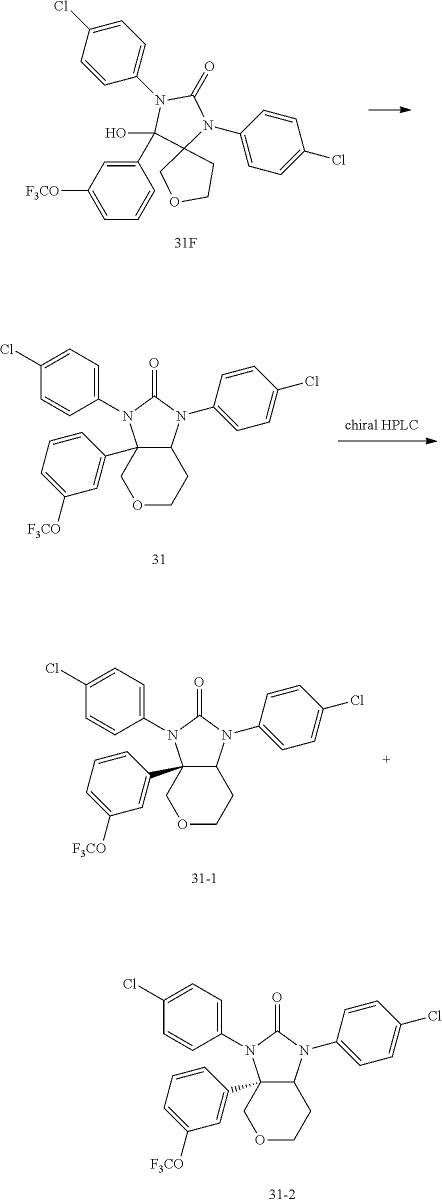 [SMILES: O=C1N(c2ccc(Cl)cc2)C2(CCOC2)C(O)(c2cccc(OC(F)(F)F)c2)N1c1ccc(Cl)cc1 ; O=C1N(c2ccc(Cl)cc2)C2CCOCC2(c2cccc(OC(F)(F)F)c2)N1c1ccc(Cl)cc1 ; O=C1N(c2ccc(Cl)cc2)C2CCOC[C@]2(c2cccc(OC(F)(F)F)c2)N1c1ccc(Cl)cc1 ; O=C1N(c2ccc(Cl)cc2)C2CCOC[C@@]2(c2cccc(OC(F)(F)F)c2)N1c1ccc(Cl)cc1]

Compounds 31B, 31C, 31D, 31E, 31F, and 31 were synthesized by employing the procedures described for Compounds 12B, 29C, 12D, 12E, 6D, and 14 using Compounds 31A, 31B, 31C at 100° C., 31D, 31E, 1-bromo-3-(trifluoromethoxy)benzene, and 31F at room temperature in lieu of Compounds 12A, 29B, 12C at 80° C., 12D, 6C, 1-bromo-3-(trifluoromethyl)benzene, and 14E at 0° C.

Compound 31B as a crude product, which was used directly in the next step without further purification. LC-MS (ESI) m/z: 240 [M−H]⁻.

Compound 31C. LC-MS (ESI) m/z: 256 [M+H]⁺.

Compound 31D. LC-MS (ESI) m/z: 409 [M+H]⁺.

Compound 31E. LC-MS (ESI) m/z: 377 [M+H]⁺.

Compound 31F. LC-MS (ESI) m/z: 539 [M+H]⁺. ¹H-NMR (400 MHz, CDCl₃): δ (ppm) 1.85-2.17 (m, 1H), 2.57-3.02 (m, 1H), 3.21-3.24 (m, 3H), 3.69-4.23 (m, 1H), 4.71-4.94 (m, 1H), 7.15-7.20 (m, 5H), 7.23-7.35 (m, 7H).

Compound 31. LC-MS (ESI) m/z: 523 [M+H]⁺. Compound 31 was separated with chiral HPLC to give Compound 31-1 and Compound 31-2.

Compound 31-1: LC-MS (ESI) m/z: 523 [M+H]⁺. ¹H-NMR (400 MHz, CDCl₃): δ (ppm) 1.87-1.99 (m, 2H), 3.65-3.72 (m, 1H), 3.79-3.84 (m, 1H), 4.09 (d, J=12.8 Hz, 1H), 4.27 (t, J=3.6 Hz, 1H), 4.60 (d, J=12.8 Hz, 1H), 7.03-7.06 (m, 2H), 7.17-7.22 (m, 3H), 7.30-7.32 (m, 2H) 7.38-7.44 (m, 3H), 7.52 (s, 1H), 7.56-7.58 (m, 1H). Chiral separation conditions: MeOH contained 0.2% NH₄OH; OZ-H (250×4.6 mm, 5 μm); retention time: 2.08 minutes.

Compound 31-2: LC-MS (ESI) m/z: 523 [M+H]⁺. ¹H-NMR (400 MHz, CDCl₃): δ (ppm) 1.91-1.98 (m, 2H), 3.65-3.71 (m, 1H), 3.79-3.84 (m, 1H), 4.09 (d, J=12.4 Hz, 1H), 4.27 (t, J=3.6 Hz, 1H), 4.60 (d, J=12.0 Hz, 1H), 7.03-7.06 (m, 2H), 7.17-7.22 (m, 3H), 7.30-7.32 (m, 2H) 7.38-7.44 (m, 3H), 7.52 (s, 1H), 7.56-7.58 (m, 1H). Chiral separation conditions: MeOH contained 0.2% NH₄OH; OZ-H (250×4.6 mm, 5 μm); retention time: 2.89 minutes.

### Example 32

### Synthesis of 3-(6-oxo-5,7-di-p-tolyl-2-oxa-5,7-diazaspiro[3.4]octan-8-yl)benzonitrile (32), (R)-3-(6-oxo-5,7-di-p-tolyl-2-oxa-5,7-diazaspiro[3.4]octan-8-yl)benzonitrile (32-1), and (S)-3-(6-oxo-5,7-di-p-tolyl-2-oxa-5,7-diazaspiro[3.4]octan-8-yl)benzonitrile (32-2)

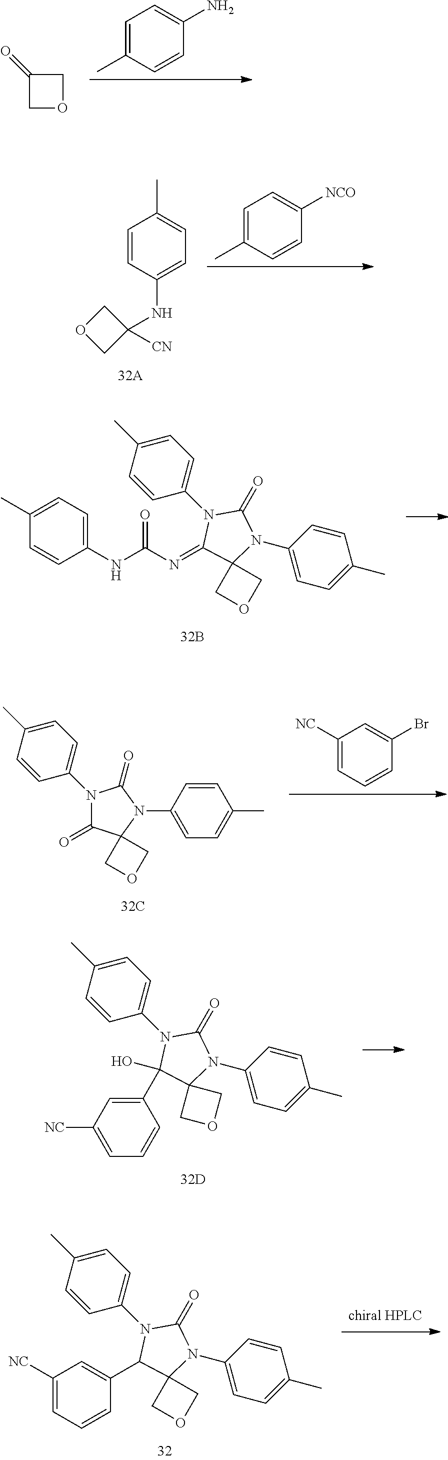

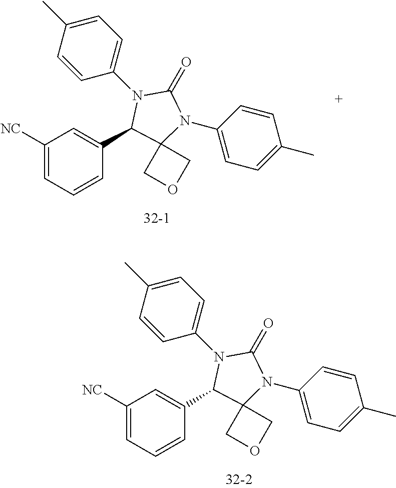 [SMILES: Cc1ccc(N2C(=O)N(c3ccc(C)cc3)C3(COC3)[C@H]2c2cccc(C#N)c2)cc1 ; Cc1ccc(N2C(=O)N(c3ccc(C)cc3)C3(COC3)[C@@H]2c2cccc(C#N)c2)cc1]

After a mixture of p-toluidine (3.21 g, 30 mmol), oxetan-3-one (4.32 g, 60 mmol), anhydrous sodium sulfate (30 g), and anhydrous MgSO₄ (10 g) in 2,2,2-trifluoroethanol (15 mL) and dichloromethane (150 mL) was stirred at room temperature for 1 hour, to it was dropped TMSCN (7.5 mL, 60 mmol) and the mixture was stirred at room temperature for 72 hours. The reaction mixture was filtered and the filtrate was concentrated under reduced pressure. The residue was purified with flash column chromatography on silica gel (ethyl acetate in petroleum ether, 9% v/v) to furnish Compound 32A. LC-MS (ESI) m/z: 189 [M+H]⁺; ¹H-NMR (CDCl₃, 400 MHz): δ (ppm) 2.28 (s, 3H), 4.21 (s, 1H), 4.72 (d, J=6.4 Hz, 2H), 5.12 (d, J=6.4 Hz, 2H), 6.46 (d, J=8.0 Hz, 2H), 7.08 (d, J=8.0 Hz, 2H).

To a solution of Compound 32A (2.0 g, 10.6 mmol) in anhydrous pyridine (30 mL) at 80° C. was added 1-isocyanato-4-methylbenzene (3.2 g, 42.4 mmol) and the mixture was stirred at 80° C. overnight. The mixture was evaporated to remove most of pyridine. The residue was triturated with methanol (20 mL), filtered, and dried to afford Compound 32B. LC-MS (ESI) m/z: 455 [M+H]⁺.

To a suspension of Compound 32B (4.0 g, 8.8 mmol) in THF (30 mL) and methanol (30 mL) was slowly added aqueous H₂SO₄ solution (2 M, 6 mL) at room temperature. The mixture was stirred at room temperature for 45 minutes and concentrated under reduced pressure. The residue was diluted with water (50 mL) and extracted with ethyl acetate (50 mL×3). The combined extracts were washed with brine (50 mL), dried over anhydrous sodium sulfate, filtered, and concentrated. The residue was purified with flash column chromatography on silica gel (ethyl acetate in petroleum ether, 40% v/v) to give Compound 32C. LC-MS (ESI) m/z: 323 [M+H]⁺; ¹H-NMR (DMSO-d₆, 400 MHz): δ (ppm) 2.35 (s, 3H), 2.39 (s, 3H), 4.67 (d, J=7.6 Hz, 2H), 4.83 (d, J=7.6 Hz, 2H), 7.29-7.38 (m, 6H), 7.47-7.49 (m, 2H).

Compounds 32D and 32 were synthesized by employing the procedures described for Compounds 6D and 6 using 3-bromobenzonitrile, Compounds 32C, and 32D at room temperature in lieu of 1-bromo-3-(trifluoromethyl)benzene, Compounds 6C, and 6D at 10° C.

Compound 32D. LC-MS (ESI) m/z: 426 [M+H]⁺; ¹H-NMR (CDCl₃, 400 MHz): δ (ppm) 2.25 (s, 3H), 2.40 (s, 3H), 3.84 (d, J=8.0 Hz, 1H), 4.44 (d, J=7.6 Hz, 1H), 4.59 (d, J=8.0 Hz, 1H), 5.60 (d, J=8.4 Hz, 1H), 5.34 (s, 1H), 6.96 (d, J=8.0 Hz, 2H), 7.06-7.17 (m, 6H), 7.41 (d, J=8.0 Hz, 1H), 7.57 (d, J=7.6 Hz, 2H), 7.84 (s, 1H).

Compound 32 was separated with chiral HPLC to yield Compound 32-1 and Compound 32-2.

Compound 32-1: LC-MS (ESI) m/z: 410 [M+H]⁺; ¹H-NMR (CDCl₃, 400 MHz): δ (ppm) 2.25 (s, 3H), 2.41 (s, 3H), 4.01 (d, J 8.4 Hz, 1H), 4.55 (d, J 8.0 Hz, 1H), 4.77 (d, J 6.8 Hz, 1H), 5.05 (d, J 7.2 Hz, 1H), 5.50 (s, 1H), 7.05 (d, J 8.4 Hz, 2H), 7.20 (d, J 8.0 Hz, 2H), 7.29-7.32 (m, 4H), 7.54 (t, J 7.6 Hz, 1H), 7.65-7.68 (m, 2H), 7.73 (s, 1H). Chiral separation conditions: MeOH contained 0.2% Methanol Ammonia; EnantioPak OD (4.6×100 mm, 5 μm); retention time: 1.16 minutes.

Compound 32-2: LC-MS (ESI) m/z: 410 [M+H]⁺; ¹H-NMR (CDCl₃, 400 MHz): δ (ppm) 2.24 (s, 3H), 2.40 (s, 3H), 4.00 (d, J 8.0 Hz, 1H), 4.54 (d, J 7.6 Hz, 1H), 4.76 (d, J 7.6 Hz, 1H), 5.03 (d, J 7.2 Hz, 1H), 5.51 (s, 1H), 7.04 (d, J 8.8 Hz, 2H), 7.20 (d, J=8.0 Hz, 2H), 7.29-7.31 (m, 4H), 7.52 (t, J=8.0 Hz, 1H), 7.63-7.66 (m, 2H), 7.72 (s, 1H). Chiral separation conditions: MeOH contained 0.2% Methanol Ammonia; EnantioPak OD (4.6×100 mm, 5 μm); retention time: 2.45 minutes.

### Example 33

### Synthesis of 3-(1,3-bis(4-chlorophenyl)-5-methyl-2-oxo-5-propylimidazolidin-4-yl)benzonitrile (33), 3-((4R,5R)-1,3-bis(4-chlorophenyl)-5-methyl-2-oxo-5-propylimidazolidin-4-yl)benzonitrile (33-1), 3-((4S,5S)-1,3-bis(4-chlorophenyl)-5-methyl-2-oxo-5-propylimidazolidin-4-yl)benzonitrile (33-2), 3-((4S,5R)-1,3-bis(4-chlorophenyl)-5-methyl-2-oxo-5-propylimidazolidin-4-yl)benzonitrile (33-3), and 3-((4R,5S)-1,3-bis(4-chlorophenyl)-5-methyl-2-oxo-5-propylimidazolidin-4-yl)benzonitrile (33-4)

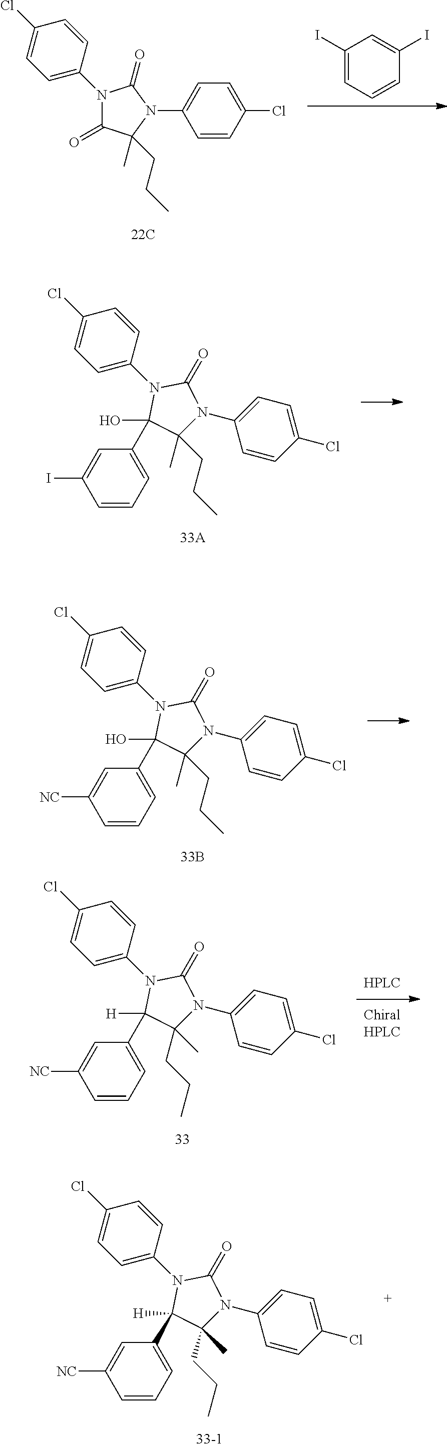 [SMILES: CCCC1(C)C(=O)N(c2ccc(Cl)cc2)C(=O)N1c1ccc(Cl)cc1 ; Ic1cccc(I)c1 ; CCCC1(C)N(c2ccc(Cl)cc2)C(=O)N(c2ccc(Cl)cc2)C1(O)c1cccc(I)c1 ; CCCC1(C)N(c2ccc(Cl)cc2)C(=O)N(c2ccc(Cl)cc2)C1(O)c1cccc(C#N)c1 ; CCCC1(C)C(c2cccc(C#N)c2)N(c2ccc(Cl)cc2)C(=O)N1c1ccc(Cl)cc1 ; CCC[C@]1(C)[C@@H](c2cccc(C#N)c2)N(c2ccc(Cl)cc2)C(=O)N1c1ccc(Cl)cc1]

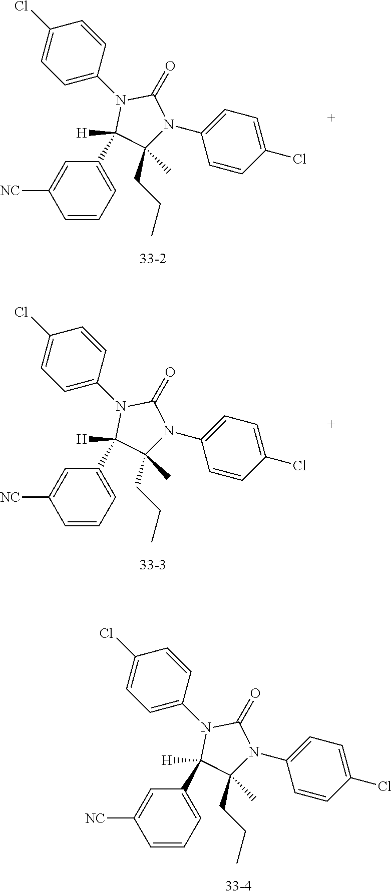 [SMILES: CCC[C@@]1(C)[C@H](c2cccc(C#N)c2)N(c2ccc(Cl)cc2)C(=O)N1c1ccc(Cl)cc1 ; CCC[C@]1(C)[C@H](c2cccc(C#N)c2)N(c2ccc(Cl)cc2)C(=O)N1c1ccc(Cl)cc1 ; CCC[C@@]1(C)[C@@H](c2cccc(C#N)c2)N(c2ccc(Cl)cc2)C(=O)N1c1ccc(Cl)cc1]

Compound 33A was synthesized by employing the procedure described for Compound 6D using 1,3-diiodobenzene and Compounds 22C at −60° C. in lieu of 1-bromo-3-(trifluoromethyl)benzene and Compound 6C at −78° C. MS (ESI) m/z: 581[M+H].

To a solution of Compound 33A (359 mg, 0.62 mmol) in DMF (8 mL) was added Pd₂(dba)₃ (57 mg, 0.062 mmol), dppf (69 mg, 0.124 mmol), and Zn(CN)₂ (109 mg, 0.93 mmol) and the mixture was stirred at 140° C. under nitrogen for 13 hours. After cooling down to room temperature, the mixture was diluted with water (5 mL) and extracted with ethyl acetate (5 mL×2). The combined extracts were dried over anhydrous sodium sulfate, filtered, and evaporated to give a crude product, which was purified by preparative HPLC to give Compound 33B. MS (ESI) m/z: 480 [M+H]; ¹H-NMR (400 MHz, CDCl₃): δ (ppm) 0.48, 0.87 (t, J=6.8 Hz, 3H), 0.85-0.88 (m, 1H), 0.93, 1.40 (s, 3H), 1.29-1.33 (m, 2H), 1.60-1.95 (m, 1H), 3.69, 3.75 (s, 1H), 7.15-7.22 (m, 4H), 7.27-7.45 (m, 5H), 7.60-7.65 (m, 2H), 7.87 (s, 1H).

Compound 33 was synthesized by employing the procedure described for Compound 6 using Compounds 33B at room temperature in lieu of Compound 6D at −10° C., which was separated with preparative HPLC followed by chiral HPLC to give Compound 33-1, Compound 33-2, Compound 33-3, and Compound 33-4.

Compound 33-1: LC-MS (ESI) m/z: 464 [M+H]⁺; ¹H-NMR (400 MHz, CDCl₃): δ (ppm) 0.75 (s, 3H), 1.00 (t, J=7.2 Hz, 3H), 1.25-1.28 (m, 2H), 1.58-1.63 (m, 2H), 5.03 (s, 1H), 7.14-7.21 (m, 4H), 7.38-7.41 (m, 4H), 7.51-7.52 (m, 2H), 7.56 (s, 1H), 7.64-7.65 (m, 1H). Chiral separation conditions: MeOH contained 0.2% Methanol Ammonia; EnantioPak AD (4.6×100 mm, 5 μm); retention time: 4.06 minutes.

Compound 33-2: LC-MS (ESI) m/z: 464 [M+H]⁺; ¹H-NMR (400 MHz, CDCl₃): δ (ppm) 0.75 (s, 3H), 1.00 (t, J=7.2 Hz, 3H), 1.25-1.28 (m, 2H), 1.57-1.63 (m, 2H), 5.03 (s, 1H), 7.14-7.21 (m, 4H), 7.38-7.41 (m, 4H), 7.51-7.52 (m, 2H), 7.56 (s, 1H), 7.63-7.64 (m, 1H). Chiral separation conditions: MeOH contained 0.2% Methanol Ammonia; EnantioPak AD (4.6×100 mm, 5 μm); retention time: 7.25 minutes.

Compound 33-3: LC-MS (ESI) m/z: 464 [M+H]⁺; ¹H-NMR (400 MHz, CDCl₃): δ (ppm) 0.51 (t, J=7.2 Hz, 3H), 0.82-0.85 (m, 1H), 1.05-1.10 (m, 2H), 1.26-1.31 (m, 1H), 1.53 (s, 3H), 4.94 (s, 1H), 7.17-7.21 (m, 4H), 7.34-7.37 (m, 2H), 7.39-7.43 (m, 2H), 7.48-7.52 (m, 1H), 7.60-7.66 (m, 3H). Chiral separation conditions: MeOH contained 0.2% Methanol Ammonia; Cellulose-SC (4.6×100 mm, 5 μm); retention time: 3.56 minutes.

Compound 33-4: LC-MS (ESI) m/z: 464 [M+H]⁺; ¹H-NMR (400 MHz, CDCl₃): δ (ppm) 0.51 (t, J=7.2 Hz, 3H), 0.78-0.85 (m, 1H), 1.06-1.07 (m, 2H), 1.26-1.28 (m, 1H), 1.53 (s, 3H), 4.93 (s, 1H), 7.17-7.21 (m, 4H), 7.34-7.37 (m, 2H), 7.40-7.42 (m, 2H), 7.50-7.52 (m, 1H), 7.60-7.66 (m, 3H). Chiral separation conditions: MeOH contained 0.2% Methanol Ammonia; Cellulose-SC (4.6×100 mm, 5 μm); retention time: 5.24 minutes.

### Example 34

### Synthesis of 3-(1,3-bis(4-chlorophenyl)-5-ethyl-5-methyl-2-oxoimidazolidin-4-yl)benzonitrile (34), 3-((4S,5S)-1,3-bis(4-chlorophenyl)-5-ethyl-5-methyl-2-oxoimidazolidin-4-yl)benzonitrile (34-1), 3-((4R,5R)-1,3-bis(4-chlorophenyl)-5-ethyl-5-methyl-2-oxoimidazolidin-4-yl)benzonitrile (34-2), 3-((4R,5S)-1,3-bis(4-chlorophenyl)-5-ethyl-5-methyl-2-oxoimidazolidin-4-yl)benzonitrile (34-3), and 3-((4S,5R)-1,3-bis(4-chlorophenyl)-5-ethyl-5-methyl-2-oxoimidazolidin-4-yl)benzonitrile (34-4)

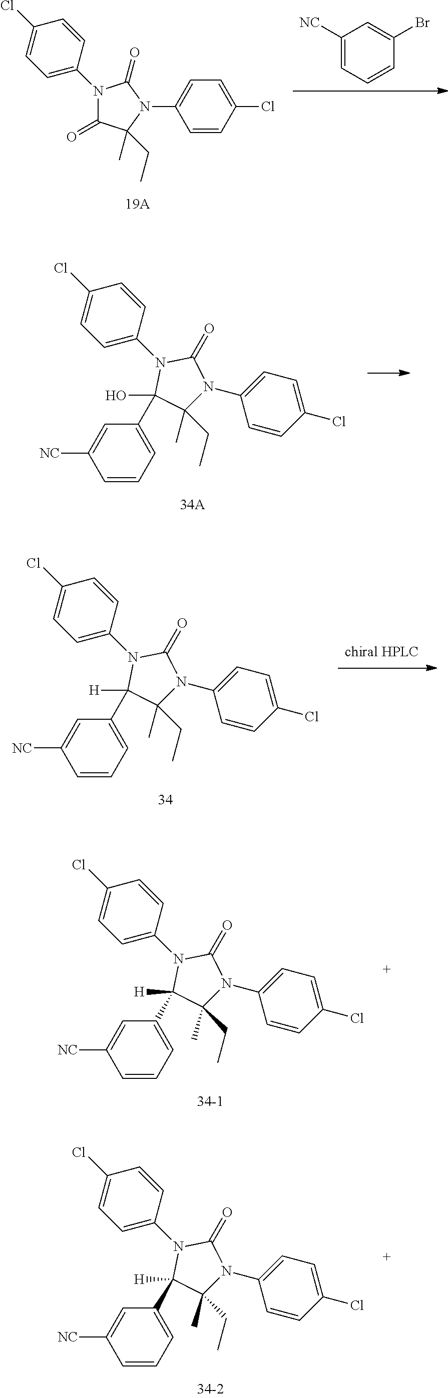 [SMILES: CCC1(C)C(=O)N(c2ccc(Cl)cc2)C(=O)N1c1ccc(Cl)cc1 ; N#Cc1cccc(Br)c1 ; CCC1(C)N(c2ccc(Cl)cc2)C(=O)N(c2ccc(Cl)cc2)C1(O)c1cccc(C#N)c1 ; CCC1(C)C(c2cccc(C#N)c2)N(c2ccc(Cl)cc2)C(=O)N1c1ccc(Cl)cc1 ; CC[C@@]1(C)[C@H](c2cccc(C#N)c2)N(c2ccc(Cl)cc2)C(=O)N1c1ccc(Cl)cc1 ; CC[C@]1(C)[C@@H](c2cccc(C#N)c2)N(c2ccc(Cl)cc2)C(=O)N1c1ccc(Cl)cc1]

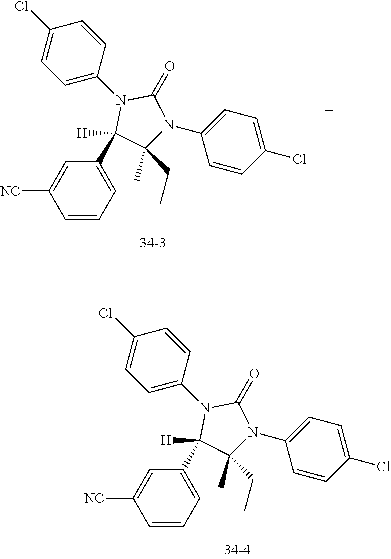 [SMILES: CC[C@@]1(C)[C@@H](c2cccc(C#N)c2)N(c2ccc(Cl)cc2)C(=O)N1c1ccc(Cl)cc1 ; CC[C@]1(C)[C@H](c2cccc(C#N)c2)N(c2ccc(Cl)cc2)C(=O)N1c1ccc(Cl)cc1]

Compounds 34A and 34 were synthesized by employing the procedures described for Compounds 6D and 6 using 3-bromobenzonitrile, Compounds 19A at −60° C., and 34A at room temperature in lieu of 1-bromo-3-(trifluoromethyl)benzene, Compounds 6C at −78° C., and 6D at 10° C.

Compound 34 was separated with chiral HPLC to give Compound 34-1, Compound 34-2, Compound 34-3, and Compound 34-4.

Compound 34-1: LC-MS (ESI) m/z: 450 [M+H]⁺; ¹H-NMR (400 MHz, CDCl₃): δ (ppm) 0.52 (t, J=7.2 Hz, 3H), 1.25-1.36 (m, 2H), 1.54 (s, 3H), 4.95 (s, 1H), 7.17-7.21 (m, 4H), 7.33-7.37 (m, 2H), 7.38-7.41 (m, 2H), 7.47-7.51 (m, 1H), 7.61-7.66 (m, 3H). Chiral separation conditions: MeOH contained 0.2% Methanol Ammonia; OJ-H (4.6×250 mm, 5 μm); retention time: 7.51 minutes.

Compound 34-2: LC-MS (ESI) m/z: 450 [M+H]⁺; ¹H-NMR (400 MHz, CDCl₃): δ (ppm) 0.53 (t, J=7.2 Hz, 3H), 1.25-1.34 (m, 2H), 1.51 (s, 3H), 4.95 (s, 1H), 7.17-7.21 (m, 4H), 7.34-7.36 (m, 2H), 7.38-7.41 (m, 2H), 7.47-7.51 (m, 1H), 7.61-7.66 (m, 3H). Chiral separation conditions: MeOH contained 0.2% Methanol Ammonia; OJ-H (4.6×250 mm, 5 μm); retention time: 6.7 minutes.

Compound 34-3: LC-MS (ESI) m/z: 450 [M+H]⁺; ¹H-NMR (400 MHz, CDCl₃): δ (ppm) 0.75 (s, 3H), 1.11 (t, J=7.2 Hz, 3H), 1.71-1.75 (m, 1H), 1.84-1.88 (m, 1H), 5.02 (s, 1H), 7.15-7.21 (m, 4H), 7.38-7.41 (m, 2H), 7.38-7.41 (m, 2H), 7.51-7.65 (m, 4H). Chiral separation conditions: MeOH (0.2% Methanol Ammonia; EnantioPak AD (4.6×100 mm, 5 μm); retention time: 1.48 minutes.

Compound 34-4: LC-MS (ESI) m/z: 450 [M+H]⁺; ¹H-NMR (400 MHz, CDCl₃): δ (ppm) 0.75 (s, 3H), 1.11 (t, J=7.2 Hz, 3H), 1.69-1.75 (m, 1H), 1.84-1.90 (m, 1H), 5.02 (s, 1H), 7.15-7.21 (m, 4H), 7.38-7.41 (m, 2H), 7.36-7.41 (m, 2H), 7.51-7.66 (m, 4H). Chiral separation conditions: MeOH (0.2% Methanol Ammonia; EnantioPak AD (4.6×100 mm, 5 μm); retention time: 2.19 minutes.

### Example 35

### Synthesis of 1,3-di-p-tolyl-4-(3-(trifluoromethoxy)phenyl)-1,3-diazaspiro[4.4]nonan-2-one (35), (R)-1,3-di-p-tolyl-4-(3-(trifluoromethoxy)phenyl)-1,3-diazaspiro[4.4]nonan-2-one (35-1), and (S)-1,3-di-p-tolyl-4-(3-(trifluoromethoxy)phenyl)-1,3-diazaspiro[4.4]nonan-2-one (35-2)

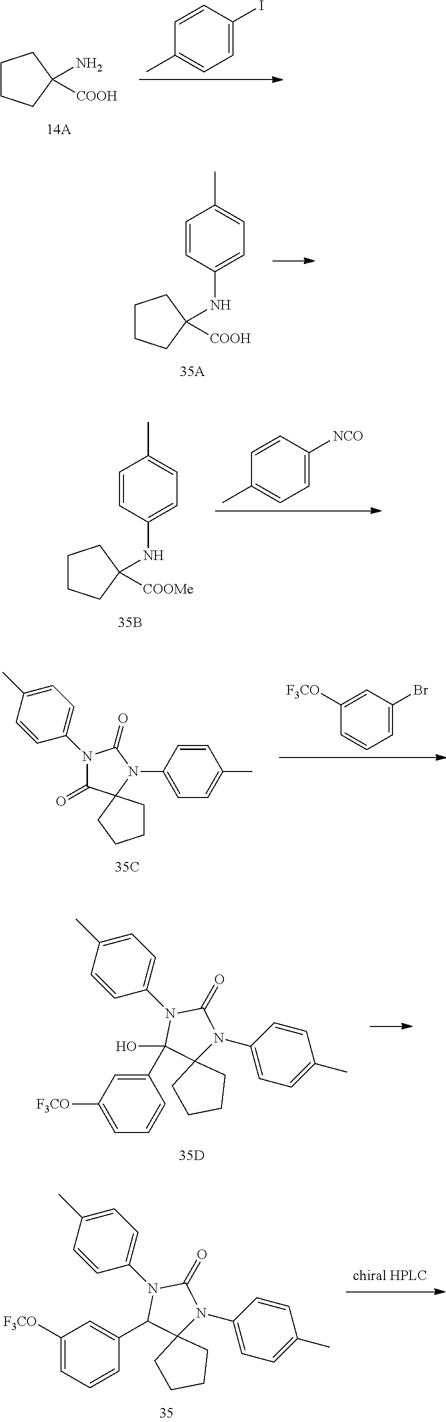

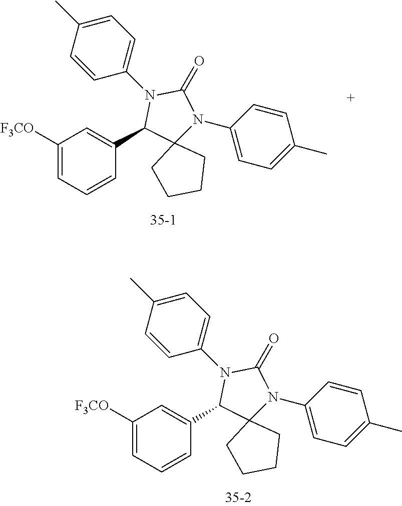 [SMILES: Cc1ccc(N2C(=O)N(c3ccc(C)cc3)C3(CCCC3)[C@H]2c2cccc(OC(F)(F)F)c2)cc1 ; Cc1ccc(N2C(=O)N(c3ccc(C)cc3)C3(CCCC3)[C@@H]2c2cccc(OC(F)(F)F)c2)cc1]

Compounds 35A, 35B, 35C, and 35D were synthesized by employing the procedures described for Compounds 12B, 12C, 14D, and 6D using Compounds 14A, 1-iodo-4-methylbenzene, 35A, 35B, 1-isocyanato-4-methylbenzene, 35C, 1-bromo-3-(trifluoromethoxy)benzene, and 35D in lieu of Compounds 12A, 1-chloro-4-iodobenzene, 12B, 1-chloro-4-isocyanatobenzene, 14C, 1-bromo-3-(trifluoromethyl)benzene, and 6C.

Compound 35A. LC-MS (ESI) m/z: 220 [M+H]⁺.

Compound 35B. LC-MS (ESI) m/z: 234 [M+H]⁺; ¹H-NMR (CDCl₃, 400 MHz): δ (ppm) 1.77-1.81 (m, 4H), 1.91-1.96 (m, 2H), 2.22 (s, 3H), 2.27-2.32 (m, 2H), 3.66 (s, 3H), 6.45 (d, J=6.8 Hz, 2H), 6.95 (d, J=6.4 Hz, 2H).

Compound 35C. LC-MS (ESI) m/z: 335 [M+H]⁺.

Compound 35D. LC-MS (ESI) m/z: 497 [M+H]⁺. ¹H-NMR (CDCl₃, 400 MHz): δ (ppm) 0.50-0.58 (m, 1H), 1.13-1.90 (m, 2H), 1.36-1.42 (m, 1H), 1.64-1.71 (m, 2H), 1.98-2.03 (m, 1H), 2.24 (s, 3H), 2.38 (s, 4H), 3.39 (s, 1H), 7.01 (d, J=6.4 Hz, 2H), 7.16-7.17 (m, 1H), 7.21-7.22 (m, 4H), 7.28-7.33 (m, 3H), 7.44-7.46 (m, 2H).

To a solution of Compound 35D (200 mg, 0.4 mmol) in AcOH (5 mL) was added Palladium on activated carbon (10%, 20 mg). The mixture was stirred at room temperature under hydrogen (1 atm.) for 12 hours. The reaction mixture was filtered and the filtrate was concentrated under reduced pressure. The residue was purified with column chromatography on silica gel (ethyl acetate in petroleum ether, 20% v/v) to furnish Compound 35. LC-MS (ESI) m/z: 481 [M+H]⁺. Compound 35 was separated with chiral HPLC to yield Compound 35-1 and Compound 35-2.

Compound 35-1: LC-MS (ESI) m/z: 481 [M+H]⁺; ¹H-NMR (CDCl₃, 400 MHz): δ (ppm) 1.09-1.14 (m, 1H), 1.26-1.42 (m, 3H), 1.49-1.62 (m, 2H), 1.95-2.06 (m, 2H), 2.23 (s, 3H), 2.37 (s, 3H), 4.86 (s, 1H), 7.01 (d, J=6.4 Hz, 2H), 7.10 (d, J=6.4 Hz, 2H), 7.17 (d, J=6.4 Hz, 1H), 7.20-7.22 (m, 3H), 7.29 (d, J=6.0 Hz, 1H), 7.36-7.41 (m, 3H). Chiral separation conditions: MeOH contained 0.2% Methanol Ammonia; OZ-H (100×4.6 mm, 5 μm); retention time: 1.22 minutes.

Compound 35-2: LC-MS (ESI) m/z: 481 [M+H]⁺; ¹H-NMR (CDCl₃, 400 MHz): δ (ppm) 1.09-1.15 (m, 1H), 1.26-1.49 (m, 3H), 1.63-1.76 (m, 2H), 1.95-2.06 (m, 2H), 2.23 (s, 3H), 2.37 (s, 3H), 4.86 (s, 1H), 7.01 (d, J=6.4 Hz, 2H), 7.10 (d, J=6.4 Hz, 2H), 7.16-7.18 (m, 1H), 7.20-7.22 (m, 3H), 7.29 (d, J=6.0 Hz, 1H), 7.36-7.41 (m, 3H). Chiral separation conditions: MeOH contained 0.2% Methanol Ammonia; OZ-H (100×4.6 mm, 5 μm); retention time: 3.18 minutes.

### Example 36

### Synthesis of 4,4′-(6-oxo-8-(3-(trifluoromethoxy)phenyl)-2-oxa-5,7-diazaspiro[3.4]octane-5,7-diyl)dibenzonitrile (36), (R)-4,4′-(6-oxo-8-(3-(trifluoromethoxy)phenyl)-2-oxa-5,7-diazaspiro[3.4]octane-5,7-diyl)dibenzonitrile (36-1) and (S)-4,4′-(6-oxo-8-(3-(trifluoromethoxy)phenyl)-2-oxa-5,7-diazaspiro[3.4]octane-5,7-diyl)dibenzonitrile (36-2)

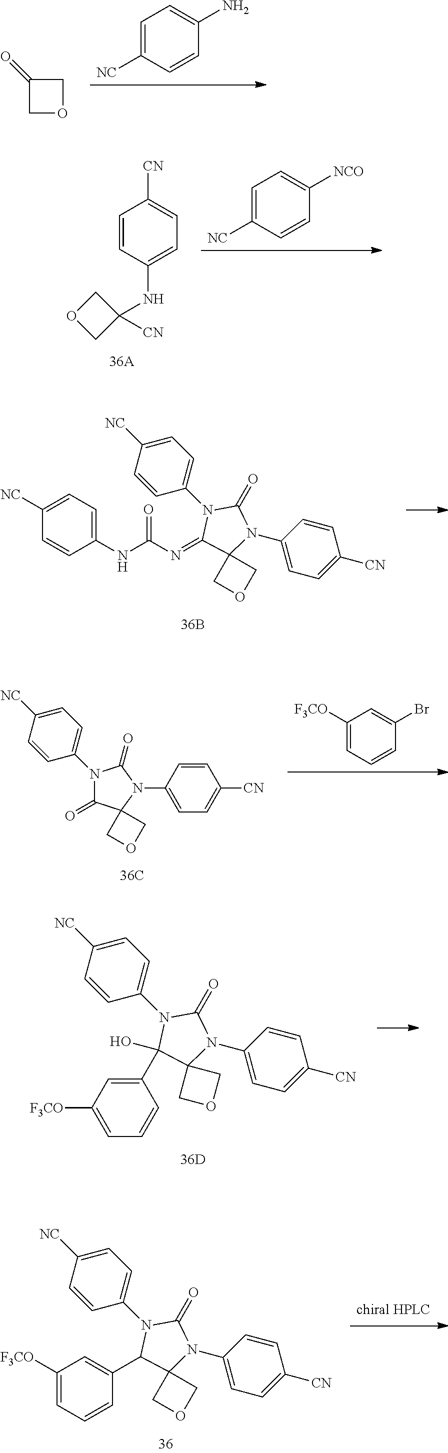

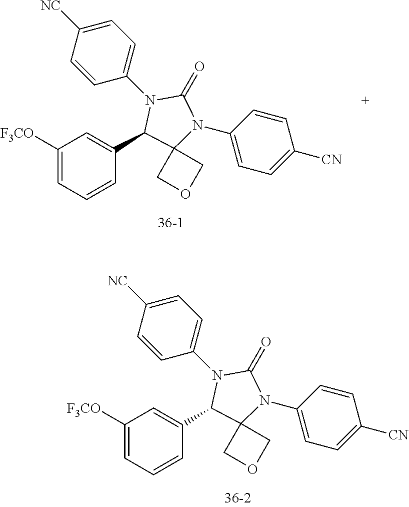 [SMILES: N#Cc1ccc(N2C(=O)N(c3ccc(C#N)cc3)C3(COC3)[C@H]2c2cccc(OC(F)(F)F)c2)cc1 ; N#Cc1ccc(N2C(=O)N(c3ccc(C#N)cc3)C3(COC3)[C@@H]2c2cccc(OC(F)(F)F)c2)cc1]

After a mixture of 4-aminobenzonitrile (1.64 g, 13.9 mmol), oxetan-3-one (2 g, 27.8 mmol), and anhydrous sodium sulfate (20 g) in dichloromethane (80 mL) was stirred at room temperature for 1 hour, to it was dropped TMSCN (3.4 mL, 27.8 mmol) and boron trifluoride diethyl etherate (3.4 mL) and the mixture was stirred at room temperature for 18 hours. The mixture was filtered and the filtrate was concentrated. The residue was purified with flash column chromatography on silica gel (ethyl acetate in petroleum ether, 55% v/v) to furnish Compound 36A. LC-MS (ESI) m/z: 200 [M+H]⁺; ¹H-NMR (CDCl₃, 400 MHz): δ (ppm) 4.73 (d, J=6.8 Hz, 2H), 4.93 (s, 1H), 5.17 (d, J=6.8 Hz, 2H), 6.55-6.58 (m, 2H), 7.56-7.58 (m, 2H).

Compounds 36B, 36C, 36D, and 36 were synthesized by employing the procedures described for Compounds 32B, 32C, 6D, and 29 using 4-isocyanatobenzonitrile, Compounds 36A, 36B, 1-bromo-3-(trifluoromethoxy)benzene, 36C, and 36D in lieu of 1-isocyanato-4-methylbenzene, Compounds 32A, 32B, 1-bromo-3-(trifluoromethyl)benzene, 6C, and 29F.

Compound 36B. LC-MS (ESI) m/z: 488 [M+H]⁺.

Compound 36C. LC-MS (ESI) m/z: 345 [M+H]⁺; ¹H-NMR (DMSO-d₆, 400 MHz): δ (ppm) 4.78 (d, J=8.4 Hz, 2H), 4.89 (d, J=8.4 Hz, 2H), 7.69-7.71 (m, 2H), 7.95-7.97 (m, 2H), 8.02-8.08 (m, 4H).

Compound 36D. LC-MS (ESI) m/z: 507 [M+H]⁺; ¹H-NMR (CDCl₃, 400 MHz): δ (ppm) 4.23 (d, J=8.4 Hz, 1H), 4.53 (s, 1H), 4.65 (d, J=8.4 Hz, 1H), 4.82 (d, J=8.8 Hz, 1H), 5.25 (d, J=8.8 Hz, 1H), 7.26-7.34 (m, 2H), 7.42-7.54 (m, 6H), 7.70 (d, J=8.4 Hz, 2H), 7.82 (d, J=8.8 Hz, 2H).

Compound 36 was separated with chiral HPLC to afford Compound 36-1 and Compound 36-2.

Compound 36-1: LC-MS (ESI) m/z: 491 [M+H]⁺; ¹H-NMR (CDCl₃, 400 MHz): δ (ppm) 4.28 (d, J 8.8 Hz, 1H), 4.53 (d, J 8.8 Hz, 1H), 4.94 (d, J 7.6 Hz, 1H), 5.10 (d, J 8.0 Hz, 1H), 5.46 (s, 1H), 7.22 (s, 1H), 7.26-7.33 (m, 2H), 7.49-7.61 (m, 5H), 7.67 (d, J 8.8 Hz, 2H), 7.82 (d, J 8.8 Hz, 2H). Chiral separation conditions: MeOH contained 0.2% Methanol Ammonia; OZ-H (4.6×100 mm, 5 μm); retention time: 0.87 minutes.

Compound 36-2: LC-MS (ESI) m/z: 491 [M+H]⁺; ¹H-NMR (CDCl₃, 400 MHz): δ (ppm) 4.28 (d, J 8.4 Hz, 1H), 4.53 (d, J 8.8 Hz, 1H), 4.95 (d, J 7.6 Hz, 1H), 5.09 (d, J 7.6 Hz, 1H), 5.48 (s, 1H), 7.22 (s, 1H), 7.27-7.34 (m, 2H), 7.49-7.61 (m, 5H), 7.67 (d, J 8.8 Hz, 2H), 7.80 (d, J 8.4 Hz, 2H). Chiral separation conditions: MeOH contained 0.2% Methanol Ammonia; OZ-H (4.6×100 mm, 5 μm); retention time: 2.86 minutes.

### Example 37

### Synthesis of 1,3-bis(4-chlorophenyl)-4-(methoxymethyl)-4-methyl-5-(3-(trifluoromethoxy)phenyl)imidazolidin-2-one (37), (4R,5R)-1,3-bis(4-chlorophenyl)-4-(methoxymethyl)-4-methyl-5-(3-(trifluoromethoxy)phenyl)imidazolidin-2-one (37-1), and (4S,5S)-1,3-bis(4-chlorophenyl)-4-(methoxymethyl)-4-methyl-5-(3-(trifluoromethoxy) phenyl)imidazolidin-2-one (37-2)

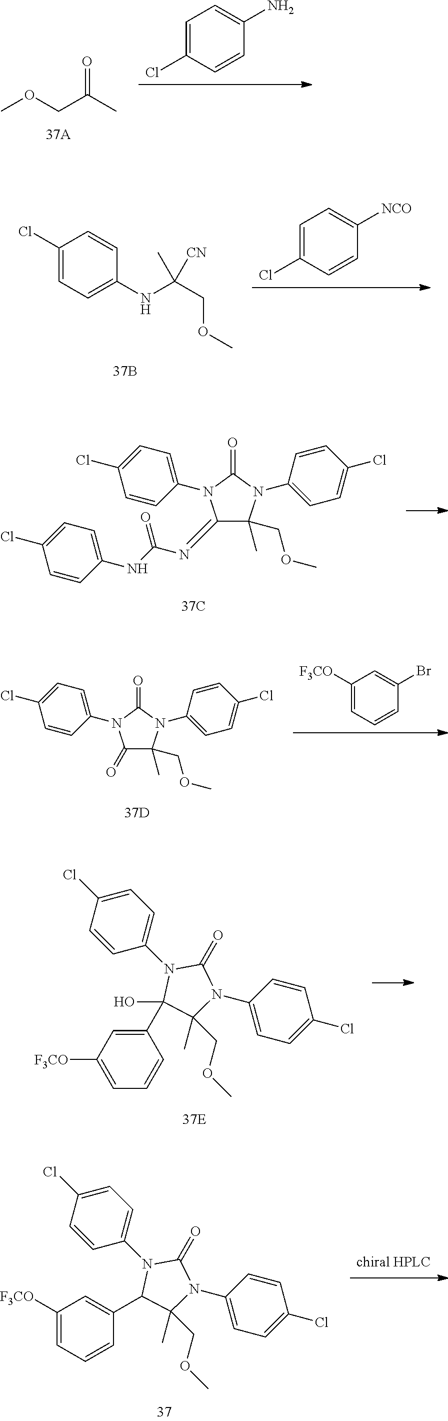

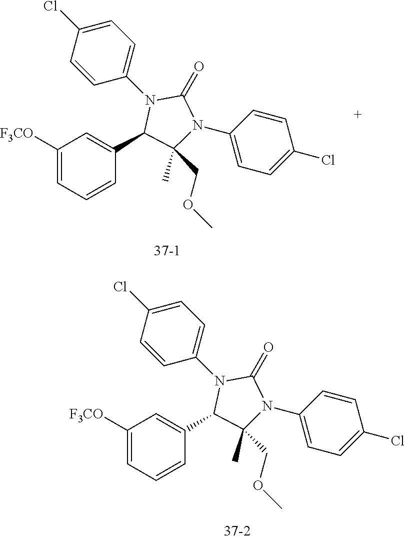 [SMILES: COC[C@@]1(C)[C@@H](c2cccc(OC(F)(F)F)c2)N(c2ccc(Cl)cc2)C(=O)N1c1ccc(Cl)cc1 ; COC[C@]1(C)[C@H](c2cccc(OC(F)(F)F)c2)N(c2ccc(Cl)cc2)C(=O)N1c1ccc(Cl)cc1]

Compounds 37B and 37C were synthesized by employing the procedures described for Compounds 32A and 32B using 4-chloroaniline, Compounds 37A without MgSO₄, 1-chloro-4-isocyanatobenzene, and 37B in lieu of p-toluidine, oxetan-3-one with MgSO₄, 1-isocyanato-4-methylbenzene, and 32A.

Compound 37B. LC-MS (ESI) m/z: 225 [M+H]⁺; ¹H-NMR (CDCl₃, 400 MHz): δ (ppm) 1.60 (s, 3H), 3.49 (s, 3H), 3.55 (d, J=9.2 Hz, 1H), 3.64 (d, J=9.2 Hz, 1H), 4.24 (s, 1H), 6.88 (d, J=8.8 Hz, 2H), 7.22 (d, J=8.8 Hz, 2H).

Compound 37C. LC-MS (ESI) m/z: 531 [M+H]⁺.

A mixture of Compound 37C (3.50 g, 6.60 mmol), methanol (50 mL) and aqueous hydrochloric acid solution (3 M, 5 mL) was stirred at 45° C. for 16 hours. The reaction mixture was diluted with brine (160 mL) and extracted with dichloromethane (100 mL×3). The combined extracts were dried over anhydrous sodium sulfate, filtered, and evaporated to give a crude product, which was purified with flash column chromatography on silica gel (methanol in dichloromethane, from 0% to 5% v/v) to afford Compound 37D. LC-MS (ESI) m/z: 379 [M+H]⁺; ¹H-NMR (CDCl₃, 400 MHz): δ (ppm) 1.45 (s, 3H), 3.23 (d, J=10.0 Hz, 1H), 3.34 (s, 3H), 3.64 (d, J=9.6 Hz, 1H), 7.29 (d, J=8.8 Hz, 2H), 7.43-7.47 (m, 6H).

Compounds 37E and 37 were synthesized by employing the procedures described for Compounds 6D and 29 using 1-bromo-3-(trifluoromethoxy)benzene, Compounds 37D, and 37E in lieu of 1-bromo-3-(trifluoromethyl)benzene, Compounds 6C, and 29F.

Compound 37E. LC-MS (ESI) m/z: 541 [M+H]⁺; ¹H-NMR (CDCl₃, 400 MHz): δ (ppm) 0.65, 1.25 (s, 3H), 2.44, 3.19 (d, J=10.4 Hz, 1H), 2.79, 3.68 (d, J=10.4 Hz, 1H), 3.06, 3.48 (s, 3H), 3.62, 5.77 (s, 1H), 7.14-7.20 (m, 3H), 7.29-7.37 (m, 3H), 7.42-7.52 (m, 6H).

Compound 37 was separated with chiral HPLC to give Compound 37-1 and Compound 37-2.

Compound 37-1: LC-MS (ESI) m/z: 525 [M+H]⁺; ¹H-NMR (CDCl₃, 400 MHz): δ (ppm) 1.47 (s, 3H), 2.65 (d, J=10.0 Hz, 1H), 2.79 (d, J=10.0 Hz, 1H), 2.92 (s, 3H), 5.08 (s, 1H), 7.16-7.24 (m, 3H), 7.25-7.41 (m, 9H); ¹H-NMR-NOESY (CDCl₃, 400 MHz): Me (1.47 ppm) has correlation with protone H (5.08 ppm). Chiral separation conditions: MeOH contained 0.2% Methanol ammonia; OZ-H (4.6×100 mm, 5 μm); retention time: 1.05 minutes.

Compound 37-2: LC-MS (ESI) m/z: 525 [M+H]⁺; ¹H-NMR (CDCl₃, 400 MHz): δ (ppm) 1.46 (s, 3H), 2.65 (d, J=10.0 Hz, 1H), 2.79 (d, J=10.0 Hz, 1H), 2.92 (s, 3H), 5.08 (s, 1H), 7.16-7.24 (m, 3H), 7.25-7.41 (m, 9H); ¹H-NMR-NOESY (CDCl₃, 400 MHz): Me (1.47 ppm) has correlation with protone H (5.08 ppm). Chiral separation conditions: MeOH contained 0.2% Methanol ammonia; OZ-H (4.6×100 mm, 5 μm); retention time: 1.93 minutes.

### Example 38

### Synthesis of 3-(1,3-bis(4-chlorophenyl)-5-(methoxymethyl)-5-methyl-2-oxoimidazolidin-4-yl)benzonitrile (38), 3-((4R,5R)-1,3-bis(4-chlorophenyl)-5-(methoxymethyl)-5-methyl-2-oxoimidazolidin-4-yl)benzonitrile (38-1), and 3-((4S,5S)-1,3-bis(4-chlorophenyl)-5-(methoxymethyl)-5-methyl-2-oxoimidazolidin-4-yl)benzonitrile (38-2)

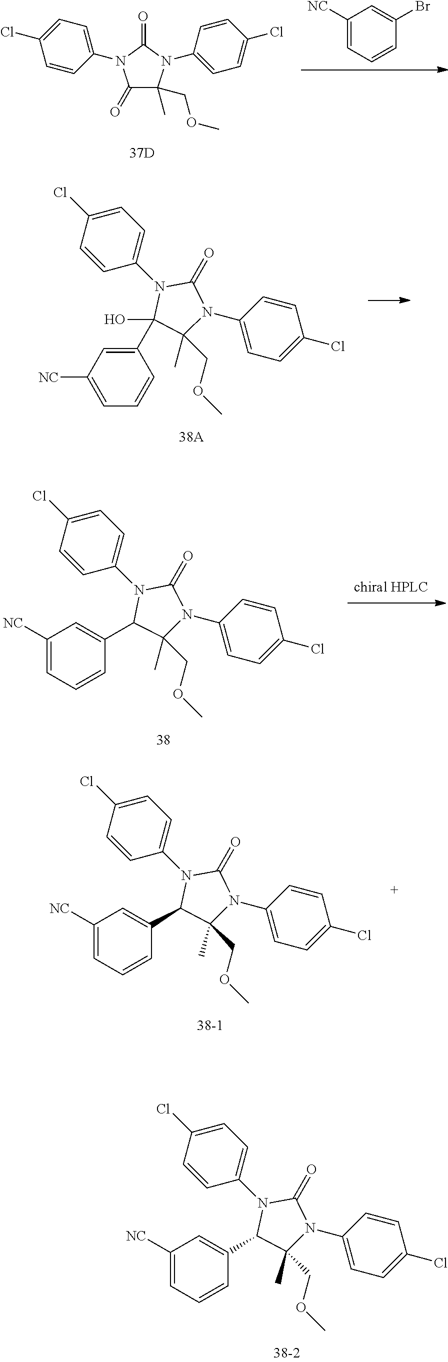 [SMILES: COCC1(C)C(=O)N(c2ccc(Cl)cc2)C(=O)N1c1ccc(Cl)cc1 ; N#Cc1cccc(Br)c1 ; COCC1(C)N(c2ccc(Cl)cc2)C(=O)N(c2ccc(Cl)cc2)C1(O)c1cccc(C#N)c1 ; COCC1(C)C(c2cccc(C#N)c2)N(c2ccc(Cl)cc2)C(=O)N1c1ccc(Cl)cc1 ; COC[C@@]1(C)[C@@H](c2cccc(C#N)c2)N(c2ccc(Cl)cc2)C(=O)N1c1ccc(Cl)cc1 ; COC[C@]1(C)[C@H](c2cccc(C#N)c2)N(c2ccc(Cl)cc2)C(=O)N1c1ccc(Cl)cc1]

Compounds 38A and 38 were synthesized by employing the procedures described for Compounds 6D and 29 using 3-bromobenzonitrile, Compounds 37D, and 38A in lieu of 1-bromo-3-(trifluoromethyl)benzene, Compounds 6C, and 29F.

Compound 38A. LC-MS (ESI) m/z: 482 [M+H]⁺; ¹H-NMR (CDCl₃, 400 MHz): δ (ppm) 0.64, 1.24 (s, 3H), 3.06, 3.49 (s, 3H), 3.20 (d, J=10.4 Hz, 1H), 3.68 (d, J=10.4 Hz, 1H), 5.88 (s, 1H), 7.15-7.18 (m, 2H), 7.23-7.30 (m, 2H), 7.38-7.46 (m, 5H), 7.62-7.70 (m, 3H).

Compound 38 was separated with chiral HPLC to give Compound 38-1 and Compound 38-2.

Compound 38-1: LC-MS (ESI) m/z: 466 [M+H]⁺; ¹H-NMR (CDCl₃, 400 MHz): δ (ppm) 1.47 (s, 3H), 2.61 (d, J=10.0 Hz, 1H), 2.79 (d, J=10.0 Hz, 1H), 2.92 (s, 3H), 5.10 (s, 1H), 7.16-7.19 (m, 2H), 7.23-7.47 (m, 7H), 7.60-7.63 (m, 2H), 7.69 (s, 1H); ¹H-NMR-NOESY (CDCl₃, 400 MHz): Me (1.47 ppm) has a correlation with protone H (5.10 ppm). Chiral separation conditions: MeOH contained 0.2% Methanol ammonia; OZ-H (4.6×100 mm, 5 μm); retention time: 1.70 minutes.

Compound 38-2: LC-MS (ESI) m/z: 466 [M+H]⁺; ¹H-NMR (CDCl₃, 400 MHz): δ (ppm) 1.47 (s, 3H), 2.59 (d, J=10.0 Hz, 1H), 2.79 (d, J=10.0 Hz, 1H), 2.92 (s, 3H), 5.10 (s, 1H), 7.16-7.19 (m, 2H), 7.23-7.46+(m, 7H), 7.57-7.63 (m, 2H), 7.69 (s, 1H); ¹H-NMR-NOESY (CDCl₃, 400 MHz): Me (1.47 ppm) has a correlation with protone H (5.10 ppm). Chiral separation conditions: MeOH contained 0.2% Methanol ammonia; OZ-H (4.6×100 mm, 5 μm); retention time: 2.38 minutes.

### Example 39

### Synthesis of 4,4′-(4-methyl-2-oxo-5-(3-(trifluoromethoxy)phenyl)-4-(trifluoromethyl)imidazolidine-1,3-diyl)dibenzonitrile (39), 4,4′-((4S,5S)-4-methyl-2-oxo-5-(3-(trifluoromethoxy)phenyl)-4-(trifluoromethyl)imidazolidine-1,3-diyl)dibenzonitrile (39-1), and 4,4′-((4R,5R)-4-methyl-2-oxo-5-(3-(trifluoromethoxy)phenyl)-4-(trifluoromethyl)imidazolidine-1,3-diyl)dibenzonitrile (39-2)

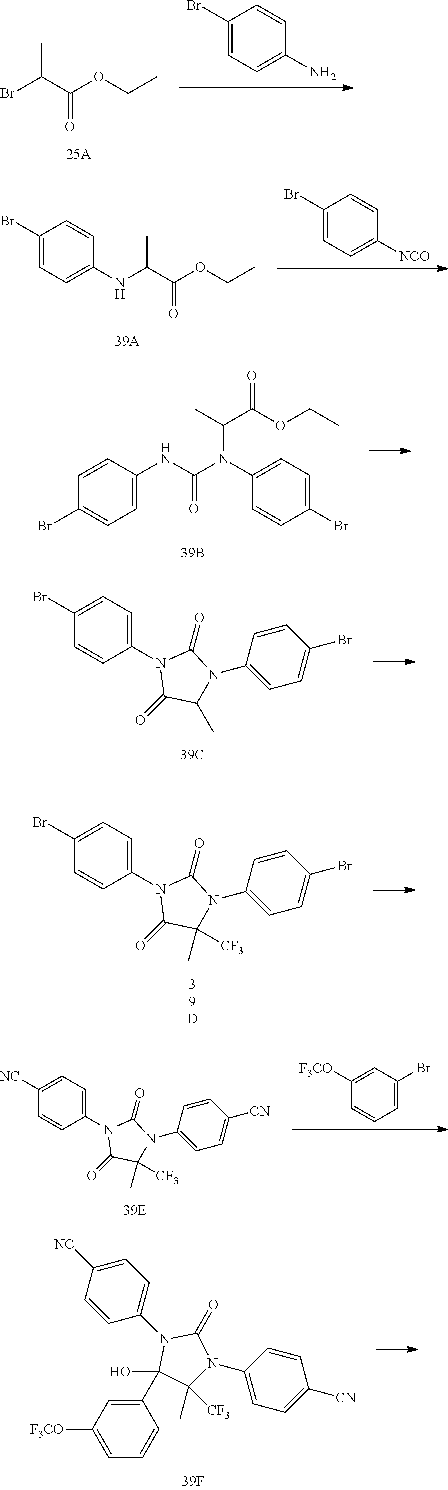

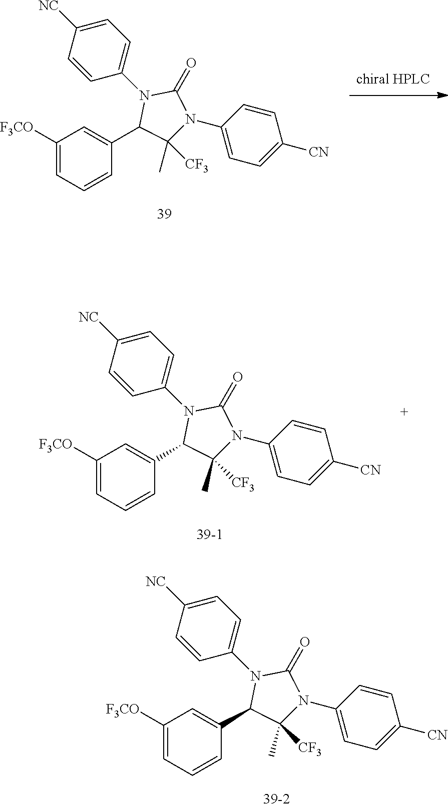 [SMILES: CC1(C(F)(F)F)C(c2cccc(OC(F)(F)F)c2)N(c2ccc(C#N)cc2)C(=O)N1c1ccc(C#N)cc1 ; C[C@@]1(C(F)(F)F)[C@H](c2cccc(OC(F)(F)F)c2)N(c2ccc(C#N)cc2)C(=O)N1c1ccc(C#N)cc1 ; C[C@]1(C(F)(F)F)[C@@H](c2cccc(OC(F)(F)F)c2)N(c2ccc(C#N)cc2)C(=O)N1c1ccc(C#N)cc1]

Compounds 39A, 39B, 39C, 39D, 39E, 39F, and 39 were synthesized by employing the procedures described for Compounds 25B, 12D, 29E, 25D, 33B, 6D, and 29 using 4-bromoaniline, Compounds 39A, 1-bromo-4-isocyanatobenzene, 39B, 39C, 39D, 39E, 1-bromo-3-(trifluoromethoxy)benzene, and 39F in lieu of 4-chloroaniline, Compounds 12C, 1-chloro-4-isocyanatobenzene, 29D, 25C, 33A, 6C, 1-bromo-3-(trifluoromethyl)benzene, and 29F.

Compound 39A. LC-MS (ESI) m/z: 272 [M+H]⁺; ¹H-NMR (CDCl₃, 400 MHz): δ (ppm) 1.27 (t, J=7.2 Hz, 3H), 1.47 (d, J=6.8 Hz, 3H), 4.05-4.12 (m, 1H), 4.12-4.17 (m, 1H), 4.20 (q, J=7.6 Hz, 2H), 6.49 (d, J=8.8 Hz, 2H), 7.26 (d, J=8.8 Hz, 2H).

Compound 39B. LC-MS (ESI) m/z: 469 [M+H]⁺.

Compound 39C. LC-MS (ESI) m/z: 423 [M+H]⁺; ¹H-NMR (CDCl₃, 400 MHz): δ (ppm) 1.60 (d, J=7.2 Hz, 3H), 4.73 (q, J=7.2 Hz, 1H), 7.38-7.41 (m, 4H), 7.58 (d, J=8.8 Hz, 2H), 7.63 (d, J=8.8 Hz, 2H).

Compound 39D. LC-MS (ESI) m/z: 491 [M+H]⁺; ¹H-NMR (CDCl₃, 400 MHz): δ (ppm) 1.83 (s, 3H), 7.22 (d, J=8.4 Hz, 2H), 7.38 (d, J=8.8 Hz, 2H), 7.63-7.66 (m, 4H).

Compound 39E. LC-MS (ESI) m/z: 385 [M+H]⁺; ¹H-NMR (CDCl₃, 400 MHz): δ (ppm) 1.91 (s, 3H), 7.50 (d, J=8.4 Hz, 2H), 7.71 (d, J=8.8 Hz, 2H), 7.82-7.86 (m, 4H).

Compound 39F. LC-MS (ESI) m/z: 547 [M+H]⁺; ¹H-NMR (CDCl₃, 400 MHz): δ (ppm) 1.10 (s, 3H), 3.85 (brs, 1H), 7.28 (s, 3H), 7.35 (d, J=8.0 Hz, 1H), 7.41 (d, J=8.4 Hz, 2H), 7.53 (s, 4H), 7.78 (d, J=8.4 Hz, 2H).

Compound 39 was separated with chiral HPLC to yield Compound 39-1 and Compound 39-2.

Compound 39-1: LC-MS (ESI) m/z: 531 [M+H]⁺; ¹H-NMR (CDCl₃, 400 MHz): δ (ppm) 1.88 (s, 3H), 5.36, 5.43 (s, 1H), 7.07-7.18 (m, 1H), 7.26 (d, J=8.4 Hz, 1H), 7.33-7.43 (m, 4H), 7.48 (d, J=8.4 Hz, 2H), 7.55 (d, J=8.8 Hz, 2H), 7.80 (d, J=8.4 Hz, 2H); ¹H-NMR-NOESY (CDCl₃, 400 MHz): Proton H (5.36-5.43 ppm) has correlation with Me (1.88 ppm). Chiral separation conditions: MeOH contained 0.2% Methanol Ammonia; S,S-Whelk-Ol (4.6×100 mm, 5 μm); retention time: 1.46 minutes.

Compound 39-2: LC-MS (ESI) m/z: 531 [M+H]⁺; ¹H-NMR (CDCl₃, 400 MHz): δ (ppm) 1.88 (s, 3H), 5.36, 5.43 (s, 1H), 7.07-7.17 (m, 1H), 7.26 (d, J=8.0 Hz, 1H), 7.33-7.43 (m, 4H), 7.48 (d, J=8.4 Hz, 2H), 7.54 (d, J=8.8 Hz, 2H), 7.80 (d, J=8.4 Hz, 2H); ¹H-NMR-NOESY (CDCl₃, 400 MHz): Proton H (5.36-5.43 ppm) has correlation with Me (1.88 ppm). Chiral separation conditions: MeOH contained 0.2% Methanol Ammonia; S,S-Whelk-Ol (4.6×100 mm 5 μm); retention time: 0.91 minutes.

### Example 40

### Synthesis of 4-(3-(4-chlorophenyl)-4,4-dimethyl-2-oxo-5-(3-(trifluoromethoxy)phenyl)imidazolidin-1-yl)benzonitrile (40), (R)-4-(3-(4-chlorophenyl)-4,4-dimethyl-2-oxo-5-(3-(trifluoromethoxy)phenyl)imidazolidin-1-yl)benzonitrile (40-1), and (S)-4-(3-(4-chlorophenyl)-4,4-dimethyl-2-oxo-5-(3-(trifluoromethoxy)phenyl)imidazolidin-1-yl)benzonitrile (40-2)

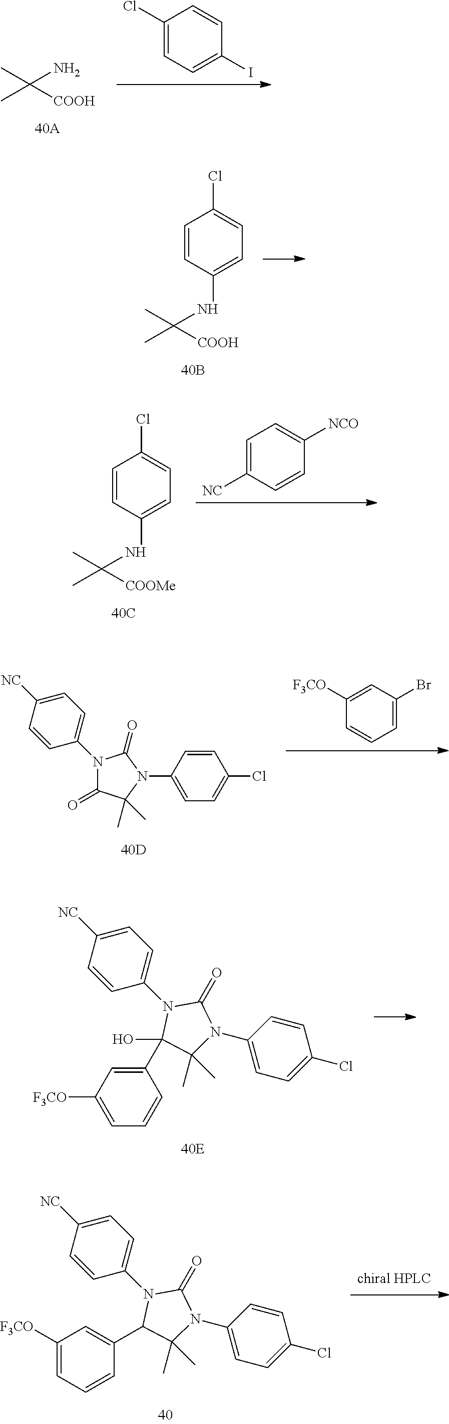

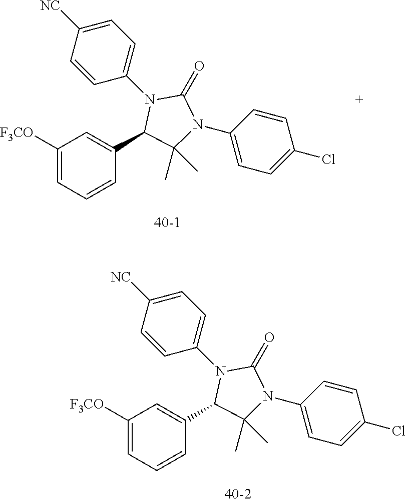 [SMILES: CC1(C)[C@@H](c2cccc(OC(F)(F)F)c2)N(c2ccc(C#N)cc2)C(=O)N1c1ccc(Cl)cc1 ; CC1(C)[C@H](c2cccc(OC(F)(F)F)c2)N(c2ccc(C#N)cc2)C(=O)N1c1ccc(Cl)cc1]

Compounds 40B, 40C, 40D, 40E, and 40 were synthesized by employing the procedures described for Compounds 12B, 12C, 14D, 6D, and 29 using Compounds 40A, 40B, 1-cyano-4-isocyanatobenzene, 40C, 40D, 1-bromo-3-(trifluoromethoxy)benzene, and 40E in lieu of Compounds 12A, 12B, 1-chloro-4-isocyanatobenzene, 14C, 6C, 1-bromo-3-(trifluoromethyl)benzene, and 29F.

Compound 40B was used directly in the next step. LC-MS (ESI) m/z: 214 [M+H]⁺.

Compound 40C. LC-MS (ESI) m/z: 228 [M+H]⁺.

Compound 40D. LC-MS (ESI) m/z: 340 [M+H]⁺. ¹H-NMR (400 MHz, CDCl₃): δ (ppm) 1.56 (s, 3H), 1.58 (s, 3H), 7.25-7.26 (m, 2H), 7.46-7.48 (m, 2H), 7.73-7.78 (m, 4H).

Compound 40E. LC-MS (ESI) m/z: 502 [M+H]⁺. ¹H-NMR (400 MHz, CDCl₃): δ (ppm) 0.78 (s, 3H), 1.29 (s, 3H), 4.57 (s, 1H), 7.10-7.19 (m, 4H), 7.23-7.26 (m, 4H), 7.43-7.45 (m, 2H), 7.52-7.54 (m, 2H).

Compound 40 was separated with chiral HPLC to afford Compound 40-1 and Compound 40-2.

Compound 40-1: LC-MS (ESI) m/z: 486 [M+H]⁺; ¹H-NMR (CDCl₃, 400 MHz): δ (ppm) 0.81 (s, 3H), 1.54 (s, 3H), 4.98 (s, 1H), 7.16-7.24 (m, 5H), 7.41-7.47 (m, 3H), 7.50-7.53 (m, 2H), 7.57-7.59 (m, 2H). Chiral separation conditions: MeOH contained 0.2% methanol ammonia; OJ (4.6×250 mm, 5 μm); retention time: 2.32 minutes.

Compound 40-2: LC-MS (ESI) m/z: 486 [M+H]⁺; ¹H-NMR (CDCl₃, 400 MHz): δ (ppm) 0.81 (s, 3H), 1.54 (s, 3H), 4.98 (s, 1H), 7.16-7.24 (m, 5H), 7.41-7.48 (m, 3H), 7.50-7.52 (m, 2H), 7.57-7.59 (m, 2H). Chiral separation conditions: MeOH contained 0.2% methanol ammonia; OJ (4.6×250 mm, 5 μm); retention time: 3.08 minutes.

### Example 41

### Synthesis of 4-(3-(4-chlorophenyl)-5,5-dimethyl-2-oxo-4-(3-(trifluoromethoxy)phenyl)imidazolidin-1-yl)benzonitrile (41), (R)-4-(3-(4-chlorophenyl)-5,5-dimethyl-2-oxo-4-(3-(trifluoromethoxy)phenyl)imidazolidin-1-yl)benzonitrile (41-1), and (S)-4-(3-(4-chlorophenyl)-5,5-dimethyl-2-oxo-4-(3-(trifluoromethoxy)phenyl)imidazolidin-1-yl)benzonitrile (41-2)

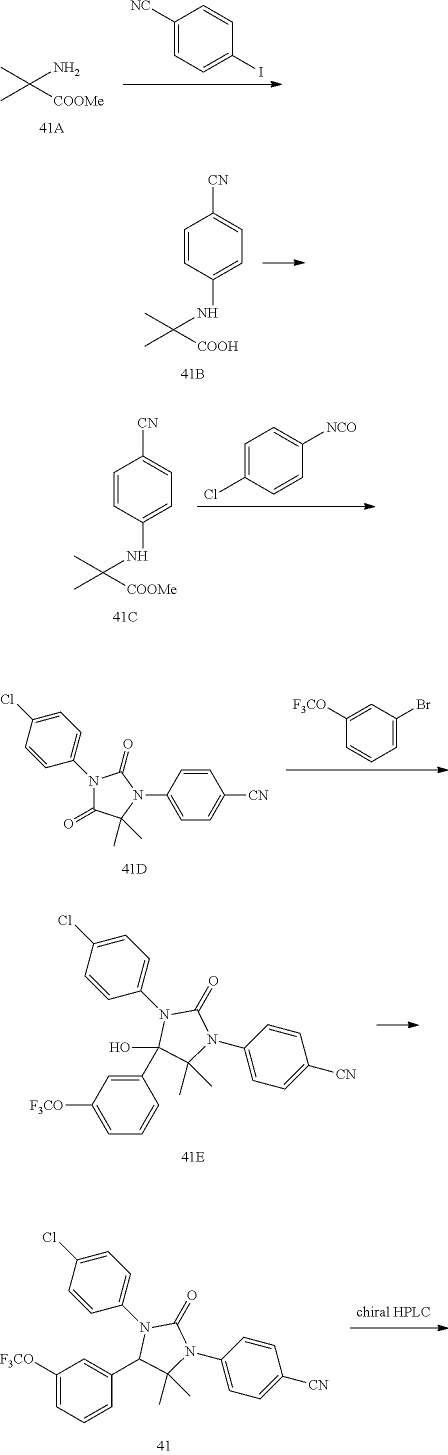

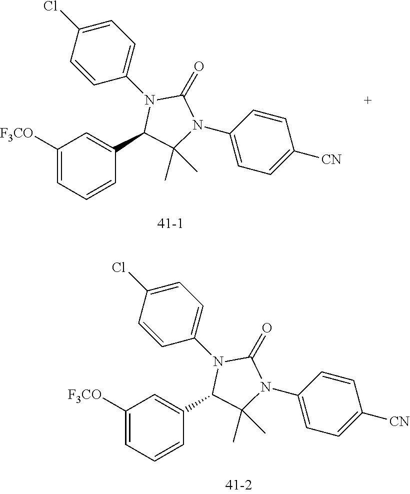 [SMILES: CC1(C)[C@@H](c2cccc(OC(F)(F)F)c2)N(c2ccc(Cl)cc2)C(=O)N1c1ccc(C#N)cc1 ; CC1(C)[C@H](c2cccc(OC(F)(F)F)c2)N(c2ccc(Cl)cc2)C(=O)N1c1ccc(C#N)cc1]

Compounds 41B, 41C, 41D, 41E, and 41 were synthesized by employing the procedures described for Compounds 12B, 12C, 14D, 6D, and 29 using 4-iodobenzonitrile, Compounds 41A, 41B, 41C, 41D, 1-bromo-3-(trifluoromethoxy)benzene, and 41E in lieu of 1-chloro-4-iodobenzene, Compounds 12A, 12B, 14C, 6C, 1-bromo-3-(trifluoromethyl)benzene, and 29F.

Compound 41B. LC-MS (ESI) m/z: 205 [M+H]⁺.

Compound 41C. LC-MS (ESI) m/z: 219 [M+H]⁺. ¹H-NMR (CDCl₃, 400 MHz): δ (ppm) 1.60 (s, 6H), 3.73 (s, 3H), 4.62 (s, 1H), 6.49 (d, J=9.2 Hz, 2H), 7.40 (d, J=8.8 Hz, 2H).

Compound 41D. LC-MS (ESI) m/z: 340 [M+H]⁺; ¹H-NMR (CDCl₃, 400 MHz): δ (ppm) 1.62 (s, 6H), 7.44-7.48 (m, 4H), 7.52-7.54 (m, 2H), 7.76-7.79 (m, 2H).

Compound 41E. LC-MS (ESI) m/z: 502 [M+H]⁺. ¹H-NMR (CDCl₃, 400 MHz): δ (ppm) 0.82 (s, 3H), 1.37 (s, 3H), 4.21 (s, 1H), 7.16-7.19 (m, 3H), 7.26-7.37 (m, 7H), 7.65 (d, J=6.8 Hz, 2H).

Compound 41. LC-MS (ESI) m/z: 486 [M+H]⁺. Compound 41 was separated with chiral HPLC to give Compound 41-1 and Compound 41-2.

Compound 41-1: LC-MS (ESI) m/z: 486 [M+H]⁺. ¹H-NMR (CDCl₃, 400 MHz): δ (ppm) 0.86 (s, 3H), 1.59 (s, 3H), 4.97 (s, 1H), 7.13 (s, 1H), 7.20-7.23 (m, 4H), 7.36 (d, J=7.2 Hz, 2H), 7.40-7.42 (m, 3H), 7.72 (d, J=6.8 Hz, 2H). Chiral separation conditions: MeOH contained 0.2% NH₄OH; OD-H (100×4.6 mm, 5 μm); retention time: 1.37 minutes.

Compound 41-2: LC-MS (ESI) m/z: 486 [M+H]⁺. ¹H-NMR (CDCl₃, 400 MHz): δ (ppm) 0.87 (s, 3H), 1.59 (s, 3H), 4.97 (s, 1H), 7.13 (s, 1H), 7.20-7.23 (m, 4H), 7.36 (d, J=7.2 Hz, 2H), 7.40-7.42 (m, 3H), 7.72 (d, J=6.8 Hz, 2H). Chiral separation conditions: MeOH contained 0.2% NH₄OH; OD-H (100×4.6 mm, 5 μm); retention time: 2.48 minutes.

### Example 42

### Synthesis of 4-(6-oxo-5-(p-tolyl)-8-(3-(trifluoromethoxy)phenyl)-2-oxa-5,7-diazaspiro[3.4]octan-7-yl)benzonitrile (42), (R)-4-(6-oxo-5-(p-tolyl)-8-(3-(trifluoromethoxy) phenyl)-2-oxa-5,7-diazaspiro[3.4]octan-7-yl)benzonitrile (42-1), and (S)-4-(6-oxo-5-(p-tolyl)-8-(3-(trifluoromethoxy)phenyl)-2-oxa-5,7-diazaspiro[3.4]octan-7-yl)benzonitrile (42-2)

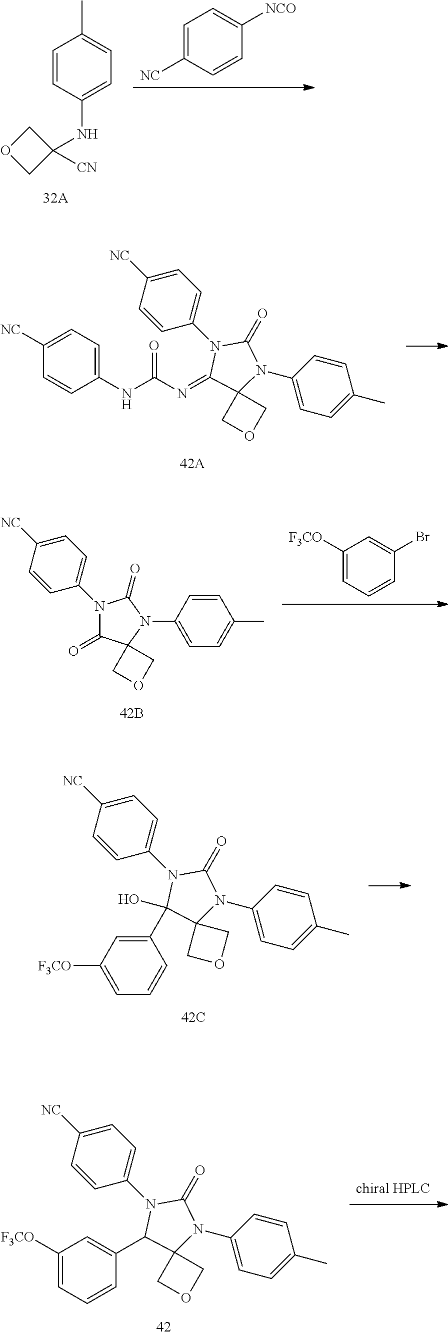

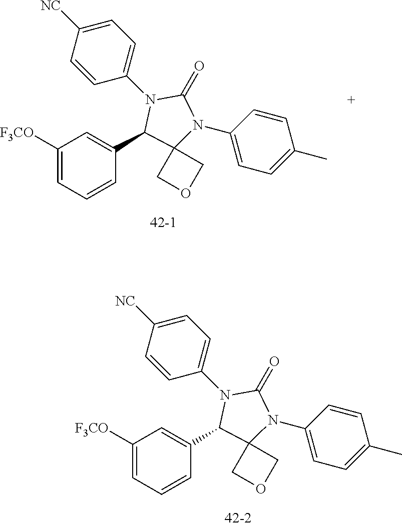 [SMILES: Cc1ccc(N2C(=O)N(c3ccc(C#N)cc3)[C@H](c3cccc(OC(F)(F)F)c3)C23COC3)cc1 ; Cc1ccc(N2C(=O)N(c3ccc(C#N)cc3)[C@@H](c3cccc(OC(F)(F)F)c3)C23COC3)cc1]

Compounds 42A, 42B, 42C, and 42 were synthesized by employing the procedures described for Compounds 32B, 32C, 6D, and 29 using 4-isocyanatobenzonitrile, Compounds 32A, 42A, 42B, 1-bromo-3-(trifluoromethoxy)benzene, and 42C in lieu of 1-isocyanato-4-methylbenzene, Compounds 32A, 32B, 6C, 1-bromo-3-(trifluoromethyl)benzene, and 29F.

Compound 42A. LC-MS (ESI) m/z: 477 [M+H]⁺.

Compound 42B. LC-MS (ESI) m/z: 334 [M+H]⁺.

Compound 42C. LC-MS (ESI) m/z: 496 [M+H]⁺; ¹H-NMR (CDCl₃, 400 MHz): δ (ppm) 2.44 (s, 3H), 3.98 (d, J=8.0 Hz, 1H), 4.50 (d, J=7.6 Hz, 1H), 4.64 (s, 1H), 4.69 (d, J=8.4 Hz, 1H), 5.15 (d, J=8.0 Hz, 1H), 7.20 (d, J=8.4 Hz, 2H), 7.24-7.30 (m, 5H), 7.41-7.43 (m, 3H), 7.51 (d, J=8.8 Hz, 2H).

Compound 42 was separated with chiral HPLC to yield Compound 42-1 and Compound 42-2.

Compound 42-1: LC-MS (ESI) m/z: 480 [M+H]⁺; ¹H-NMR (CDCl₃, 400 MHz): δ (ppm) 2.44 (s, 3H), 4.11 (d, J=8.0 Hz, 1H), 4.52 (d, J=8.4 Hz, 1H), 4.81 (d, J=7.6 Hz, 1H), 5.06 (d, J=7.6 Hz, 1H), 5.51 (s, 1H), 7.20 (d, J=8.4 Hz, 2H), 7.29 (d, J=8.8 Hz, 2H), 7.35 (d, J=8.0 Hz, 2H), 7.39 (d, J=8.0 Hz, 1H), 7.51-7.56 (m, 3H), 7.65 (d, J=8.8 Hz, 2H). Chiral separation conditions: n-Hexane/EtOH contained 0.1% DEA (70/30); OJ-H (4.6×250 mm, 5 μm); retention time: 13.65 minutes.

Compound 42-2: LC-MS (ESI) m/z: 480 [M+H]⁺; ¹H-NMR (CDCl₃, 400 MHz): δ (ppm) 2.44 (s, 3H), 4.11 (d, J=8.0 Hz, 1H), 4.52 (d, J=8.4 Hz, 1H), 4.81 (d, J=7.2 Hz, 1H), 5.06 (d, J=7.6 Hz, 1H), 5.52 (s, 1H), 7.20 (d, J=8.4 Hz, 2H), 7.29 (d, J=8.8 Hz, 2H), 7.35 (d, J=8.0 Hz, 2H), 7.39 (d, J=8.0 Hz, 1H), 7.51-7.56 (m, 3H), 7.65 (d, J=8.8 Hz, 2H). Chiral separation conditions: n-Hexane/EtOH contained 0.1% DEA (70/30); OJ-H (4.6×250 mm, 5 μm); retention time: 22.31 minutes.

### Example 43

### Synthesis of 4-(6-oxo-7-(p-tolyl)-8-(3-(trifluoromethoxy)phenyl)-2-oxa-5,7-diazaspiro[3.4]octan-5-yl)benzonitrile (43), (R)-4-(6-oxo-7-(p-tolyl)-8-(3-(trifluoromethoxy) phenyl)-2-oxa-5,7-diazaspiro[3.4]octan-5-yl)benzonitrile (43-1), and (S)-4-(6-oxo-7-(p-tolyl)-8-(3-(trifluoromethoxy)phenyl)-2-oxa-5,7-diazaspiro[3.4]octan-5-yl)benzonitrile (43-2)

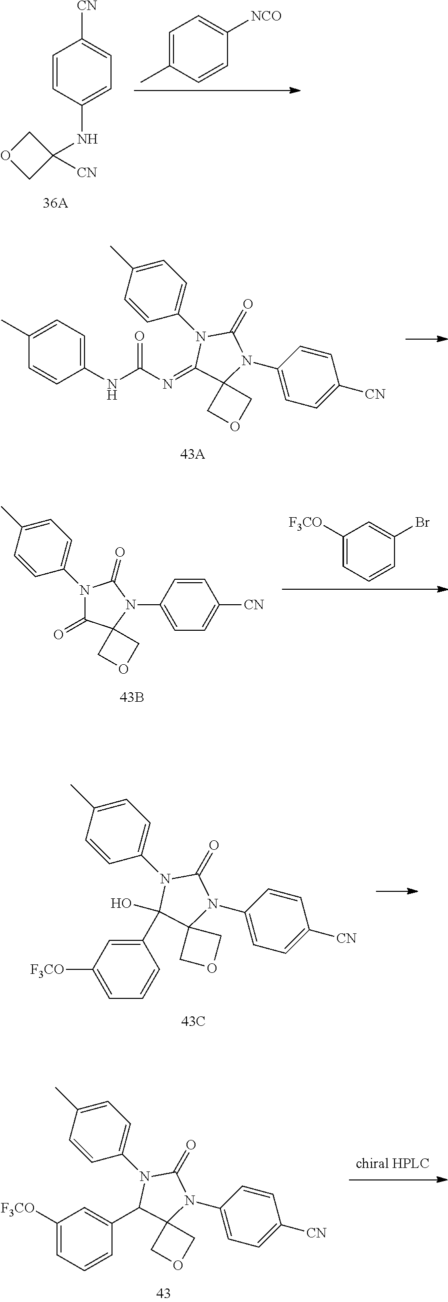

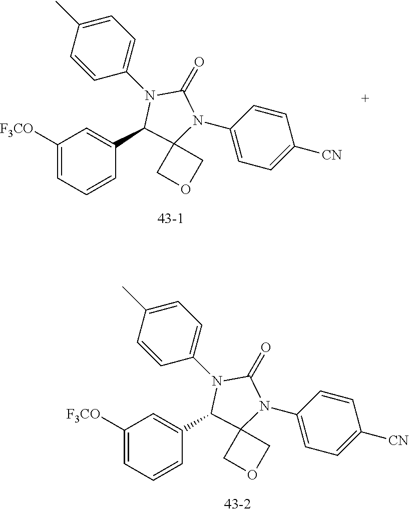 [SMILES: Cc1ccc(N2C(=O)N(c3ccc(C#N)cc3)C3(COC3)[C@H]2c2cccc(OC(F)(F)F)c2)cc1 ; Cc1ccc(N2C(=O)N(c3ccc(C#N)cc3)C3(COC3)[C@@H]2c2cccc(OC(F)(F)F)c2)cc1]

Compounds 43A, 43B, 43C, and 43 were synthesized by employing the procedures described for Compounds 32B, 32C, 6D, and 29 using Compounds 36A, 43A, 43B, 1-bromo-3-(trifluoromethoxy)benzene, and 43C in lieu of Compounds 32A, 32B, 6C, 1-bromo-3-(trifluoromethyl)benzene, and 29F.

Compound 43A. LC-MS (ESI) m/z: 466 [M+H]⁺.

Compound 43B. LC-MS (ESI) m/z: 334 [M+H]⁺; ¹H-NMR (CDCl₃, 400 MHz): δ (ppm) 2.41 (s, 3H), 5.03 (d, J=7.6 Hz, 2H), 5.25 (d, J=8.0 Hz, 2H), 7.31 (s, 4H), 7.82 (d, J=8.8 Hz, 2H), 8.05 (d, J=8.8 Hz, 2H).

Compound 43C. LC-MS (ESI) m/z: 496 [M+H]⁺; ¹H-NMR (CDCl₃, 400 MHz): δ (ppm) 2.27 (s, 3H), 4.15 (s, 1H), 4.20 (d, J=8.0 Hz, 1H), 4.58 (d, J=8.4 Hz, 1H), 4.77 (d, J=8.8 Hz, 1H), 5.26 (d, J=8.8 Hz, 1H), 7.04 (d, J=8.0 Hz, 2H), 7.17 (d, J=8.4 Hz, 2H), 7.22-7.25 (m, 1H), 7.38 (s, 1H), 7.44 (d, J=4.8 Hz, 2H), 7.68-7.71 (m, 2H), 7.77-7.79 (m, 2H).

Compound 43 was separated with chiral HPLC to yield Compound 43-1 and Compound 43-2.

Compound 43-1: LC-MS (ESI) m/z: 480 [M+H]⁺; ¹H-NMR (CDCl₃, 400 MHz): δ (ppm) 2.26 (s, 3H), 4.29 (d, J=8.8 Hz, 1H), 4.59 (d, J=8.8 Hz, 1H), 4.94 (d, J=8.0 Hz, 1H), 5.13 (d, J=7.6 Hz, 1H), 5.39 (s, 1H), 7.07 (d, J=8.4 Hz, 2H), 7.21-7.31 (m, 5H), 7.45 (t, J=8.0 Hz, 1H), 7.73-7.79 (m, 4H). Chiral separation conditions: MeOH contained 0.2% Methanol Ammonia; Cellulose-SC (4.6×100 mm, 5 μm); retention time: 1.07 minutes.

Compound 43-2: LC-MS (ESI) m/z: 480 [M+H]⁺; ¹H-NMR (CDCl₃, 400 MHz): δ (ppm) 2.26 (s, 3H), 4.29 (d, J=8.4 Hz, 1H), 4.59 (d, J=8.4 Hz, 1H), 4.94 (d, J=7.6 Hz, 1H), 5.12 (d, J=8.0 Hz, 1H), 5.39 (s, 1H), 7.07 (d, J=8.0 Hz, 2H), 7.21-7.31 (m, 5H), 7.45 (t, J=7.6 Hz, 1H), 7.73-7.78 (m, 4H); Chiral separation conditions: MeOH contained 0.2% Methanol Ammonia; Cellulose-SC (4.6×100 mm, 5 μm); retention time: 1.33 minutes.

### Example 44

### Synthesis of 4-(3-(4-bromophenyl)-5-methyl-2-oxo-4-(3-(trifluoromethoxy) phenyl)-5-(trifluoromethyl)imidazolidin-1-yl)benzonitrile (44), 4-((4S,5S)-3-(4-bromophenyl)-5-methyl-2-oxo-4-(3-(trifluoromethoxy)phenyl)-5-(trifluoromethyl) imidazolidin-1-yl)benzonitrile (44-1) and 4-((4R,5R)-3-(4-bromophenyl)-5-methyl-2-oxo-4-(3-(trifluoromethoxy)phenyl)-5-(trifluoromethyl)imidazolidin-1-yl)benzonitrile (44-2)

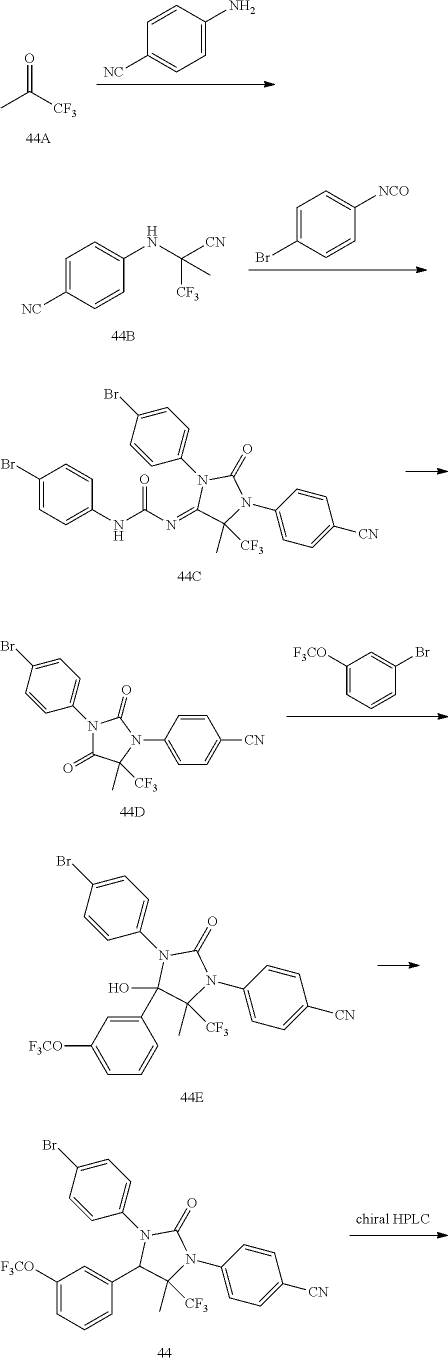

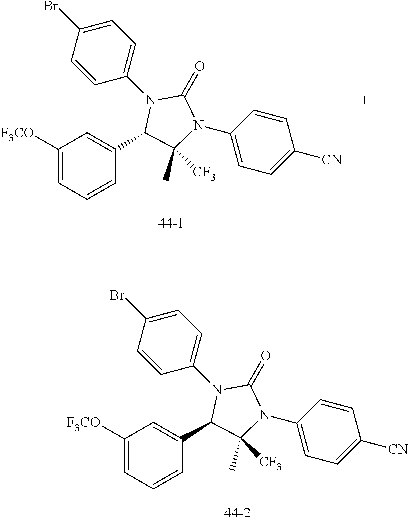

Compounds 44B, 44C, 44D, 44E, and 44 were synthesized by employing the procedures described for Compounds 36A, 32B, 32C, 6D, and 6 using Compounds 44A, 44B, 1-bromo-4-isocyanatobenzene, 44C, 44D, 1-bromo-3-(trifluoromethoxy)benzene, and 44E in lieu of oxetan-3-one, 32A, 1-isocyanato-4-methylbenzene, 32B, 6C, 1-bromo-3-(trifluoromethyl)benzene, and 6D.

Compound 44B. LC-MS (ESI) m/z: 240 [M+H]⁺; ¹H-NMR (CDCl₃, 400 MHz): δ (ppm) 1.96 (s, 3H), 4.38 (s, 1H), 7.03 (d, J=8.8 Hz, 2H), 7.55-7.58 (m, 2H).

Compound 44C. LC-MS (ESI) m/z: 634 [M+H]⁺; ¹H-NMR (CDCl₃, 400 MHz): δ (ppm) 1.92 (s, 3H), 6.85-6.90 (m, 3H), 7.15-7.17 (m, 2H), 7.36-7.48 (m, 6H), 7.80 (d, J=8.4 Hz, 2H).

Compound 44D. LC-MS (ESI) m/z: 438 [M+H]⁺; ¹H-NMR (CDCl₃, 400 MHz): δ (ppm) 1.87 (s, 3H), 7.35-7.37 (m, 2H), 7.46-7.48 (m, 2H), 7.63-7.65 (m, 2H), 7.80-7.82 (m, 2H).

Compound 44E. LC-MS (ESI) m/z: 600 [M+H]⁺; ¹H-NMR (CDCl₃, 500 MHz): δ (ppm) 1.07 (s, 3H), 3.83 (s, 1H), 7.21-7.24 (m, 2H), 7.26 (s, 2H), 7.29-7.31 (m, 1H), 7.37-7.40 (m, 4H), 7.48 (s, 1H), 7.73-7.74 (m, 2H).

Compound 44 was separated with chiral HPLC to yield Compound 44-1 and Compound 44-2.

Compound 44-1: LC-MS (ESI) m/z: 584 [M+H]⁺; ¹H-NMR (CDCl₃, 400 MHz): δ (ppm) 1.85 (s, 3H), 5.33 (s, 1H), 7.09-7.15 (m, 3H), 7.21 (d, J 8.0 Hz, 2H), 7.35-7.39 (m, 3H), 7.45-7.47 (m, 2H), 7.75-7.78 (m, 2H); COSY (CDCl₃, 400 MHz): Me (1.85 ppm) has correlation with Proton H (5.33 ppm). Chiral separation conditions: MeOH contained 0.2% Methanol Ammonia; OJ-H (4.6×100 mm, 5 μm); retention time: 1.28 minutes.

Compound 44-2: LC-MS (ESI) m/z: 584 [M+H]⁺; ¹H-NMR (CDCl₃, 400 MHz): δ (ppm) 1.85 (s, 3H), 5.33 (s, 1H), 7.09-7.15 (m, 3H), 7.21 (d, J 7.6 Hz, 2H), 7.35-7.38 (m, 3H), 7.45 (d, J 8.4 Hz, 2H), 7.75-7.78 (m, 2H). COSY (CDCl₃, 400 MHz): Me (1.85 ppm) has correlation with Proton H (5.33 ppm). Chiral separation conditions: MeOH contained 0.2% Methanol Ammonia; OJ-H (4.6×100 mm, 5 μm); retention time: 1.89 minutes.

### Example 45

### Synthesis of 3-(5,7-bis(4-chlorophenyl)-2,2-difluoro-6-oxo-5,7-diazaspiro[3.4]octan-8-yl)benzonitrile (45), (R)-3-(5,7-bis(4-chlorophenyl)-2,2-difluoro-6-oxo-5,7-diazaspiro[3.4]octan-8-yl)benzonitrile (45-1), and (S)-3-(5,7-bis(4-chlorophenyl)-2,2-difluoro-6-oxo-5,7-diazaspiro[3.4]octan-8-yl)benzonitrile (45-2)

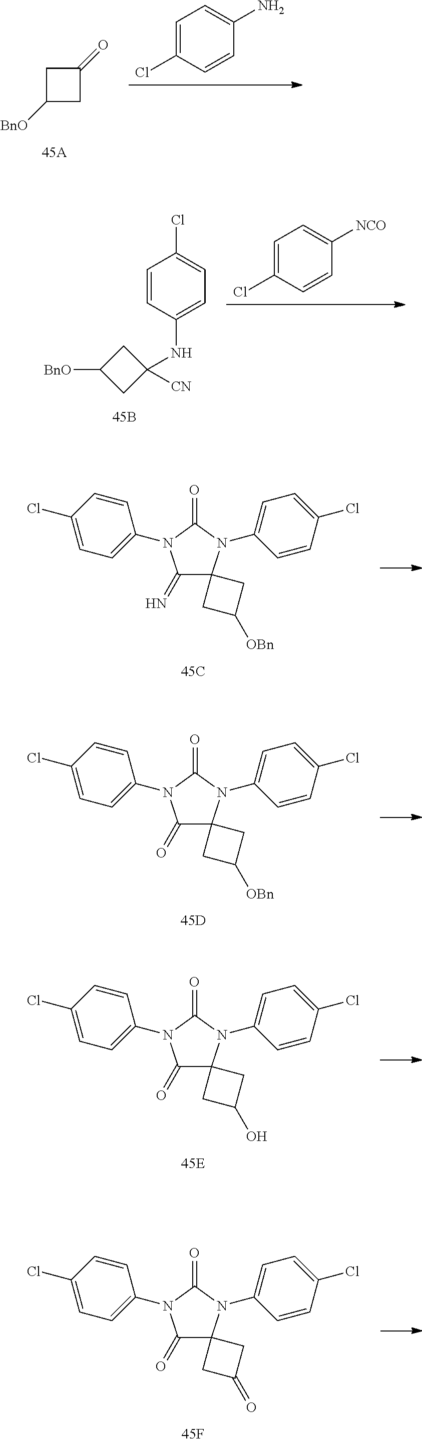

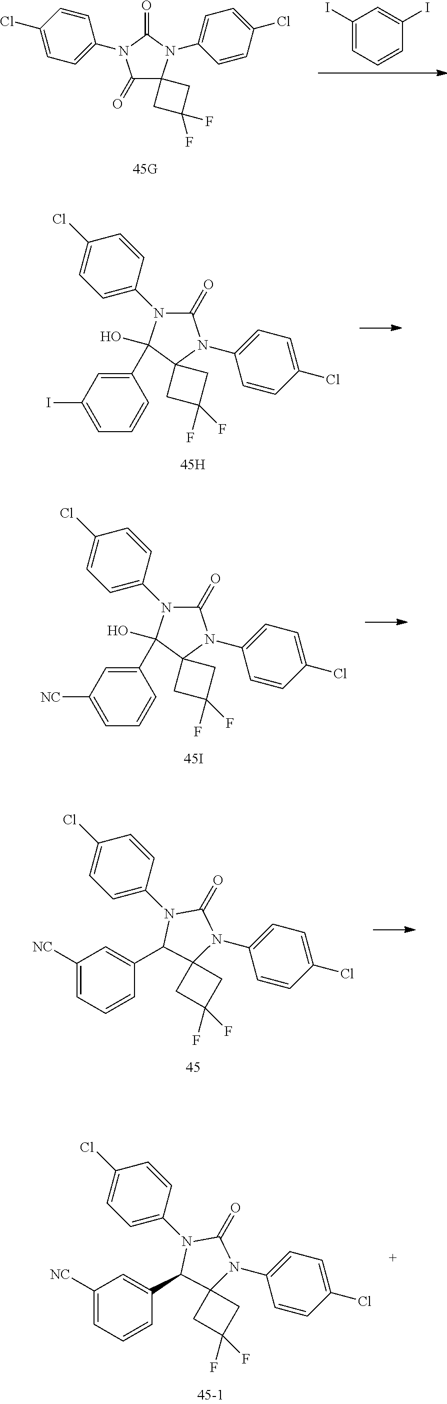 [SMILES: O=C1N(c2ccc(Cl)cc2)C(=O)C2(CC(F)(F)C2)N1c1ccc(Cl)cc1 ; Ic1cccc(I)c1 ; O=C1N(c2ccc(Cl)cc2)C2(CC(F)(F)C2)C(O)(c2cccc(I)c2)N1c1ccc(Cl)cc1 ; N#Cc1cccc(C2(O)N(c3ccc(Cl)cc3)C(=O)N(c3ccc(Cl)cc3)C23CC(F)(F)C3)c1 ; N#Cc1cccc(C2N(c3ccc(Cl)cc3)C(=O)N(c3ccc(Cl)cc3)C23CC(F)(F)C3)c1 ; N#Cc1cccc([C@H]2N(c3ccc(Cl)cc3)C(=O)N(c3ccc(Cl)cc3)C23CC(F)(F)C3)c1]

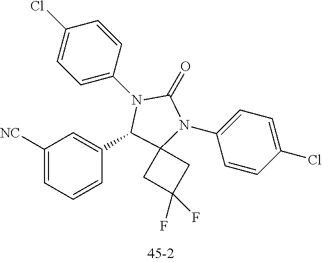 [SMILES: N#Cc1cccc([C@@H]2N(c3ccc(Cl)cc3)C(=O)N(c3ccc(Cl)cc3)C23CC(F)(F)C3)c1]

Compounds 45B, 45C, and 45D were synthesized by employing the procedures described for Compounds 32A, 32B, and 37D using 4-chloroaniline, Compounds 45A, 45B, 1-chloro-4-isocyanatobenzene, and 45C in lieu of p-toluidine, oxetan-3-one, 32A, 1-isocyanato-4-methylbenzene, and 37C.

Compound 45B. LC-MS (ESI) m/z: 313 [M+H]⁺; ¹H-NMR (400 MHz, CDCl₃): δ (ppm) 2.33-2.38 (m, 2H), 3.12-3.17 (m, 2H), 4.13 (s, 1H), 4.26-4.30 (m, 1H), 4.47 (s, 2H), 6.55-6.59 (m, 2H), 7.19-7.22 (m, 2H), 7.30-7.36 (m, 5H).

Compound 45C. LC-MS (ESI) m/z: 466 [M+H]⁺.

Compound 45D. LC-MS (ESI) m/z: 467 [M+H]⁺; ¹H-NMR (400 MHz, CDCl₃): δ (ppm) 2.33-2.38 (m, 2H), 2.82-2.86 (m, 2H), 4.34-4.37 (m, 1H), 4.40 (s, 2H), 7.23-7.26 (m, 4H), 7.29-7.34 (m, 3H), 7.43 (s, 4H), 7.48-7.50 (m, 2H).

To a solution of Compound 45D (4.6 g, 10 mmol) in dichloromethane (100 mL) was dropped TMSI (4.0 g, 20 mmol). The mixture was stirred at room temperature overnight and quenched with saturated sodium hydrogen sulfite solution. The organic layer was dried over anhydrous sodium sulfate, filtered, and evaporated to give a crude product, which was purified with column chromatography on silica gel (ethyl acetate in petroleum ether, 30% v/v) to furnish Compound 45E. LC-MS (ESI) m/z: 377 [M+H]⁺. ¹H-NMR (400 MHz, CDCl₃): δ (ppm) 1.97 (d, J=5.6 Hz, 1H), 2.30-2.36 (m, 2H), 2.87-2.92 (m, 2H), 4.63-4.67 (m, 1H), 7.24-7.27 (m, 2H), 7.44-7.53 (m, 6H).

A mixture of Compound 45E (377 mg, 1.0 mmol) and DMP (636 mg, 1.5 mmol) in dichloromethane (5 mL) was stirred at room temperature overnight. The reaction mixture was washed with saturated sodium hydrogen sulfite solution (5 mL), dried over anhydrous sodium sulfate, filtered, and concentrated. The crude product was purified by flash column chromatography on silica gel (ethyl acetate in petroleum ether, 20% v/v) to yield Compound 45F. LC-MS (ESI) m/z: 375 [M+H]⁺; ¹H-NMR (400 MHz, CDCl₃): δ (ppm) 3.47-3.52 (m, 2H), 3.79-3.84 (m, 2H), 7.31-7.33 (m, 2H), 7.48-7.49 (m, 4H), 7.51-7.53 (m, 2H).

A mixture of Compound 45F (750 mg, 2.0 mmol) and BAST (5 mL) was stirred at 60° C. for 5 hours. After cooling down to room temperature, the mixture was dropped into ice-water (20 mL) and extracted with ethyl acetate (10 mL×2). The combined extracts were dried over anhydrous sodium sulfate, filtered, and evaporated to give a crude product, which was purified by flash column chromatography on silica gel (ethyl acetate in petroleum ether, 20% v/v) to furnish to afford Compound 45G. LC-MS (ESI) m/z: 397 [M+H]⁺; ¹H-NMR (400 MHz, CDCl₃): δ (ppm) 3.03-3.10 (m, 2H), 3.37-3.44 (m, 2H), 7.28-7.31 (m, 2H), 7.45-7.48 (m, 4H), 7.51-7.54 (m, 2H).

Compounds 45H, 45I, and 45 were synthesized by employing the procedures described for Compounds 6D, 33B, and 6 using 1,3-diiodobenzene, Compounds 45G at −60° C., 45H, and 45I at room temperature in lieu of 1-bromo-3-(trifluoromethyl)benzene, Compounds 6C at −78° C., 33A, and 6D at 10° C.

Compound 45H. LC-MS (ESI) m/z: 601 [M+H]⁺; ¹H-NMR (400 MHz, CDCl₃): δ (ppm) 2.34-2.37 (m, 1H), 2.74-2.78 (m, 1H), 2.88-2.92 (m, 1H), 3.48-3.52 (m, 1H), 7.12-7.16 (m, 1H), 7.22-7.26 (m, 2H), 7.48-7.52 (m, 5H), 7.55-7.58 (m, 2H), 7.71-7.73 (m, 1H), 7.98 (br, 1H).

Compound 45I. MS (ESI) m/z: 500 [M+H]; ¹H-NMR (400 MHz, CDCl₃): δ (ppm) 2.16-2.26 (m, 1H), 2.50-2.57 (m, 1H), 2.72-2.80 (m, 1H), 3.32-3.40 (m, 1H), 5.18 (s, 1H), 7.05-7.07 (m, 2H), 7.16-7.21 (m, 4H), 7.33-7.35 (m, 2H), 7.42-7.52 (m, 2H), 7.61-7.78 (m, 2H).

Compound 45 was separated with chiral HPLC to give Compound 45-1 and Compound 45-2.

Compound 45-1: LC-MS (ESI) m/z: 484 [M+H]⁺; ¹H-NMR (CDCl₃, 400 MHz): δ (ppm) 2.17-2.23 (m, 1H), 2.48-2.60 (m, 1H), 2.86-2.93 (m, 2H), 5.26 (d, J=2.0 Hz, 1H), 7.11-7.15 (m, 2H), 7.20-7.24 (m, 2H), 7.40-7.44 (m, 2H), 7.47-7.50 (m, 2H), 7.55-7.60 (m, 2H), 7.64 (s, 1H), 7.68-7.71 (m, 1H). Chiral separation conditions: MeOH contained 0.2% Methanol Ammonia; OZ-H (100×4.6 mm, 5 μm); retention time: 1.15 minutes.

Compound 45-2: LC-MS (ESI) m/z: 484 [M+H]⁺; ¹H-NMR (CDCl₃, 400 MHz): δ (ppm) 2.17-2.23 (m, 1H), 2.51-2.56 (m, 1H), 2.86-2.92 (m, 2H), 5.26 (d, J=2.0 Hz, 1H), 7.11-7.14 (m, 2H), 7.20-7.24 (m, 2H), 7.40-7.43 (m, 2H), 7.48-7.50 (m, 2H), 7.55-7.60 (m, 2H), 7.64 (s, 1H), 7.68-7.71 (m, 1H). Chiral separation conditions: MeOH contained 0.2% Methanol Ammonia; OZ-H (100×4.6 mm, 5 μm); retention time: 2.19 minutes.

### Example 46

### Synthesis of 4-(7-(4-chlorophenyl)-6-oxo-8-(3-(trifluoromethoxy)phenyl)-2-oxa-5,7-diazaspiro[3.4]octan-5-yl)benzonitrile (46), (R)-4-(7-(4-chlorophenyl)-6-oxo-8-(3-(trifluoromethoxy)phenyl)-2-oxa-5,7-diazaspiro[3.4]octan-5-yl)benzonitrile (46-1) and (S)-4-(7-(4-chlorophenyl)-6-oxo-8-(3-(trifluoromethoxy)phenyl)-2-oxa-5,7-diazaspiro[3.4]octan-5-yl)benzonitrile (46-2)

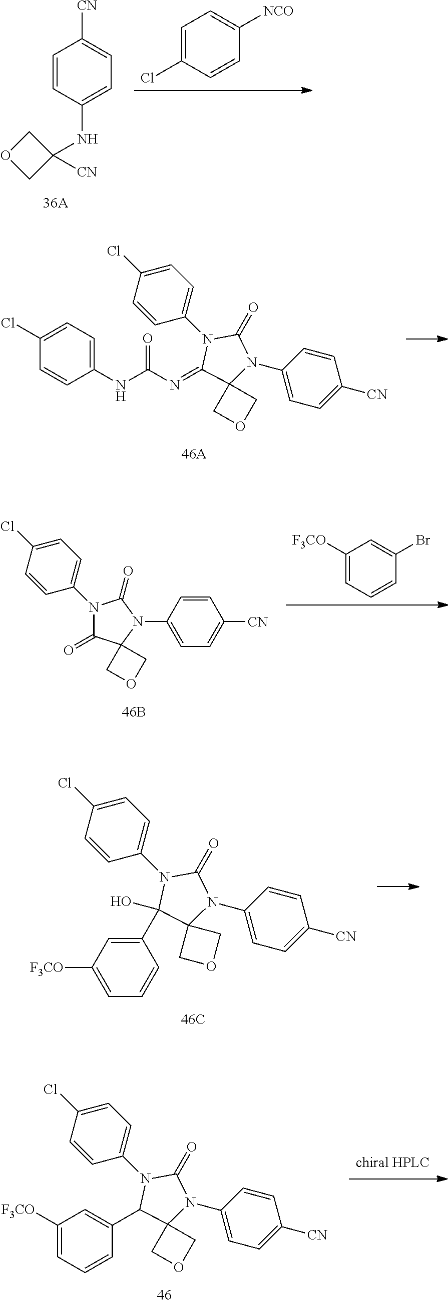

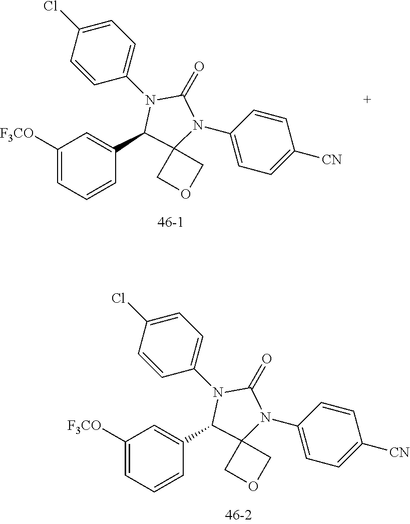 [SMILES: N#Cc1ccc(N2C(=O)N(c3ccc(Cl)cc3)[C@H](c3cccc(OC(F)(F)F)c3)C23COC3)cc1 ; N#Cc1ccc(N2C(=O)N(c3ccc(Cl)cc3)[C@@H](c3cccc(OC(F)(F)F)c3)C23COC3)cc1]

Compounds 46A, 46B, 46C, and 46 were synthesized by employing the procedures described for Compounds 32B, 32C, 6D, and 29 using 1-chloro-4-isocyanatobenzene, Compounds 36A, 46A, 46B, 1-bromo-3-(trifluoromethoxy)benzene, and 46C in lieu of 1-isocyanato-4-methylbenzene, Compounds 32A, 32B, 6C, 1-bromo-3-(trifluoromethyl)benzene, and 29F.

Compound 46A. LC-MS (ESI) m/z: 506 [M+H]⁺.

Compound 46B. LC-MS (ESI) m/z: 354 [M+H]⁺.

Compound 46C. LC-MS (ESI) m/z: 516 [M+H]⁺; ¹H-NMR (CD₃OD, 500 MHz): 4.12 (d, J=8.5 Hz, 1H), 4.64 (d, J=8.5 Hz, 1H), 4.77 (d, J=8.5 Hz, 1H), 5.27 (d, J=9.0 Hz, 1H), 7.23 (d, J=15 Hz, 2H), 7.30 (d, J=8.0 Hz, 1H), 7.47-7.54 (m, 4H), 7.60-7.63 (m, 1H), 7.91-7.95 (m, 4H).

Compound 46 was separated with chiral HPLC to give Compound 46-1 and Compound 46-2.

Compound 46-1: LC-MS (ESI) m/z: 500 [M+H]⁺; ¹H-NMR (CD₃OD, 500 MHz): δ (ppm) 4.22 (d, J=9.0 Hz, 1H), 4.54 (d, J=8.5 Hz, 1H), 5.04 (d, J=8.0 Hz, 1H), 5.08 (d, J=8.0 Hz, 1H), 5.90 (s, 1H), 7.28-7.29 (m, 2H), 7.31 (d, J=8.5 Hz, 1H), 7.42 (s, 1H), 7.49 (d, J=8.5 Hz, 3H), 7.55 (t, J=8.0 Hz, 1H), 7.82 (d, J=8.5 Hz, 2H), 7.92 (d, J=8.5 Hz, 2H). Chiral separation conditions: MeOH contained 0.2% Methanol Ammonia; R,R-Whelk-Ol (4.6×100 mm, 5 μm); retention time: 2.01 minutes.

Compound 46-2: LC-MS (ESI) m/z: 500 [M+H]⁺; ¹H-NMR (CD₃OD, 500 MHz): δ (ppm) 4.22 (d, J=9.0 Hz, 1H), 4.54 (d, J=8.5 Hz, 1H), 5.04 (d, J=8.0 Hz, 1H), 5.08 (d, J=8.0 Hz, 1H), 5.90 (s, 1H), 7.28-7.29 (m, 2H), 7.31 (d, J=8.5 Hz, 1H), 7.42 (s, 1H), 7.49 (d, J=8.5 Hz, 3H), 7.55 (t, J=8.0 Hz, 1H), 7.82 (d, J=8.5 Hz, 2H), 7.92 (d, J=8.5 Hz, 2H). Chiral separation conditions: MeOH contained 0.2% Methanol Ammonia; R,R-Whelk-Ol (4.6×100 mm, 5 μm); retention time: 3.62 minutes.

### Example 47

### Synthesis of 4-(5-(4-chlorophenyl)-6-oxo-8-(3-(trifluoromethoxy)phenyl)-2-oxa-5,7-diazaspiro[3.4]octan-7-yl)benzonitrile (47), (R)-4-(5-(4-chlorophenyl)-6-oxo-8-(3-(trifluoromethoxy)phenyl)-2-oxa-5,7-diazaspiro[3.4]octan-7-yl)benzonitrile (47-1), and (S)-4-(5-(4-chlorophenyl)-6-oxo-8-(3-(trifluoromethoxy)phenyl)-2-oxa-5,7-diazaspiro[3.4]octan-7-yl)benzonitrile (47-2)

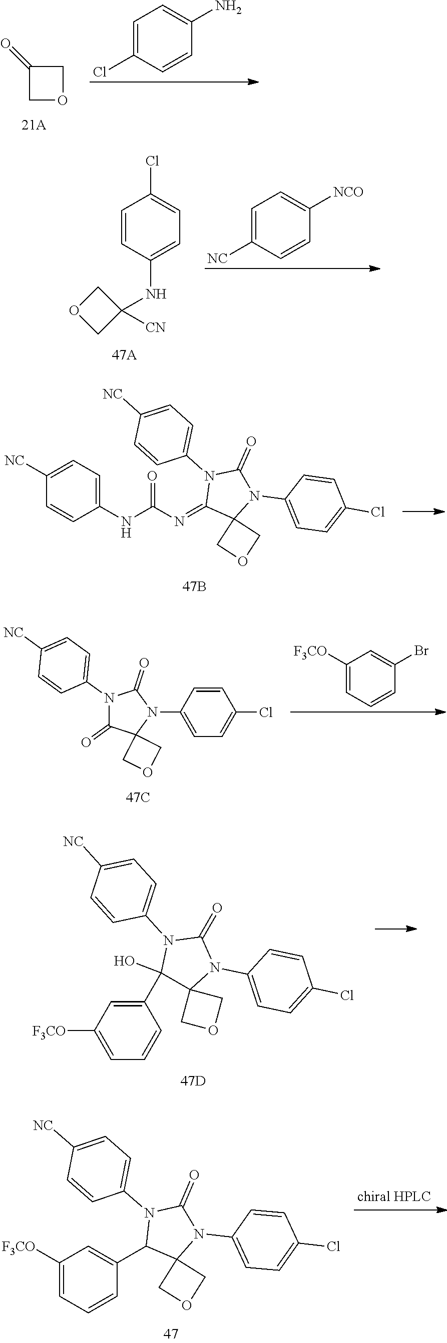

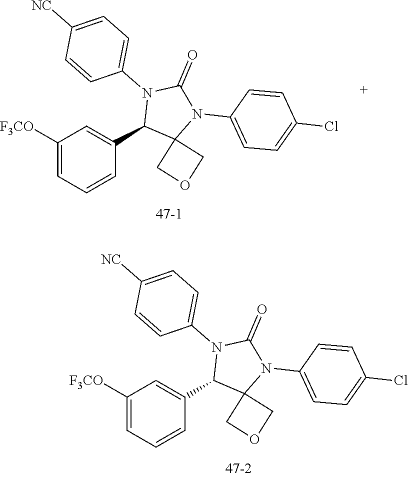 [SMILES: N#Cc1ccc(N2C(=O)N(c3ccc(Cl)cc3)C3(COC3)[C@H]2c2cccc(OC(F)(F)F)c2)cc1 ; N#Cc1ccc(N2C(=O)N(c3ccc(Cl)cc3)C3(COC3)[C@@H]2c2cccc(OC(F)(F)F)c2)cc1]

Compounds 47A, 47B, 47C, 47D, and 47 were synthesized by employing the procedures described for Compounds 32A, 32B, 32C, 6D, and 29 using 4-chloroaniline, 4-isocyanatobenzonitrile, Compounds 47A, 47B, 47C, 1-bromo-3-(trifluoromethoxy)benzene, and 47D in lieu of p-toluidine, 1-isocyanato-4-methylbenzene, Compounds 32A, 32B, 6C, 1-bromo-3-(trifluoromethyl)benzene, and 29F.

Compound 47A. LC-MS (ESI) m/z: 209 [M+H]⁺; ¹H-NMR (CDCl₃, 400 MHz): δ (ppm) 4.36 (s, 1H), 4.72 (d, J=6.4 Hz, 2H), 5.12 (d, J=6.4 Hz, 2H), 6.48 (d, J=9.2 Hz, 2H), 7.25 (d, J=9.2 Hz, 2H).

Compound 47B. LC-MS (ESI) m/z: 497 [M+H]⁺; ¹H-NMR (DMSO-d₆, 500 MHz): δ (ppm) 4.79 (d, J=6.4 Hz, 2H), 5.07 (d, J=6.0 Hz, 2H), 7.46 (d, J=6.4 Hz, 2H), 7.59 (d, J=6.4 Hz, 2H), 7.65-7.71 (m, 6H), 7.83 (d, J=6.8 Hz, 2H), 10.16 (s, 1H).

Compound 47C. LC-MS (ESI) m/z: 354 [M+H]⁺; ¹H-NMR (DMSO-d₆, 500 MHz): δ (ppm) 4.68 (d, J=6.0 Hz, 2H), 4.85 (d, J=6.4 Hz, 2H), 7.66 (s, 4H), 7.71 (t, J=5.6 Hz, 2H), 8.01 (q, J=4 Hz, 2H).

Compound 47D. LC-MS (ESI) m/z: 516 [M+H]⁺; ¹H-NMR (DMSO-d₆, 400 MHz): δ (ppm) 3.73 (d, J=8.4 Hz, 1H), 4.42 (d, J=8.0 Hz, 1H), 4.53 (d, J=8.4 Hz, 1H), 5.08 (d, J=8.4 Hz, 1H), 7.35-7.69 (m, 12H), 8.04 (s, 1H).

Compound 47 was separated with chiral HPLC to afford Compound 47-1 and Compound 47-2.

Compound 47-1: LC-MS (ESI) m/z: 500 [M+H]⁺; ¹H-NMR (CDCl₃, 400 MHz): δ (ppm) 4.14 (d, J=8 Hz, 1H), 4.47 (d, J=8 Hz, 1H), 4.82 (d, J=7.2 Hz, 1H), 5.01 (d, J=7.2 Hz, 1H), 5.49 (s, 1H), 7.23 (s, 1H), 7.27 (t, J=8.8 Hz, 3H), 7.34 (d, J=7.6 Hz, 1H), 7.49-7.54 (m, 5H), 7.61 (d, J=9.2 Hz, 2H). Chiral separation conditions: MeOH contained 0.2% Methanol Ammonia; OD-H (4.6×100 mm, 5 μm); retention time: 0.98 minutes.

Compound 47-2: LC-MS (ESI) m/z: 500 [M+H]⁺; ¹H-NMR (CDCl₃, 400 MHz): δ (ppm) 4.14 (d, J=8 Hz, 1H), 4.47 (d, J=8 Hz, 1H), 4.82 (d, J=7.6 Hz, 1H), 5.01 (d, J=7.6 Hz, 1H), 5.49 (s, 1H), 7.23 (s, 1H), 7.28 (q, J=6.8 Hz, 3H), 7.34 (d, J=7.6 Hz, 1H), 7.49-7.54 (m, 5H), 7.61 (d, J=9.2 Hz, 2H). Chiral separation conditions: MeOH contained 0.2% Methanol Ammonia; OD-H (4.6×100 mm, 5 μm); retention time: 1.36 minutes.

### Example 48

### Synthesis of 3-(3-(4-cyanophenyl)-5,5-dimethyl-2-oxo-1-(p-tolyl) imidazolidin-4-yl)benzonitrile (48), (R)-3-(3-(4-cyanophenyl)-5,5-dimethyl-2-oxo-1-(p-tolyl)imidazolidin-4-yl)benzonitrile (48-1), and (S)-3-(3-(4-cyanophenyl)-5,5-dimethyl-2-oxo-1-(p-tolyl)imidazolidin-4-yl)benzonitrile (48-2)

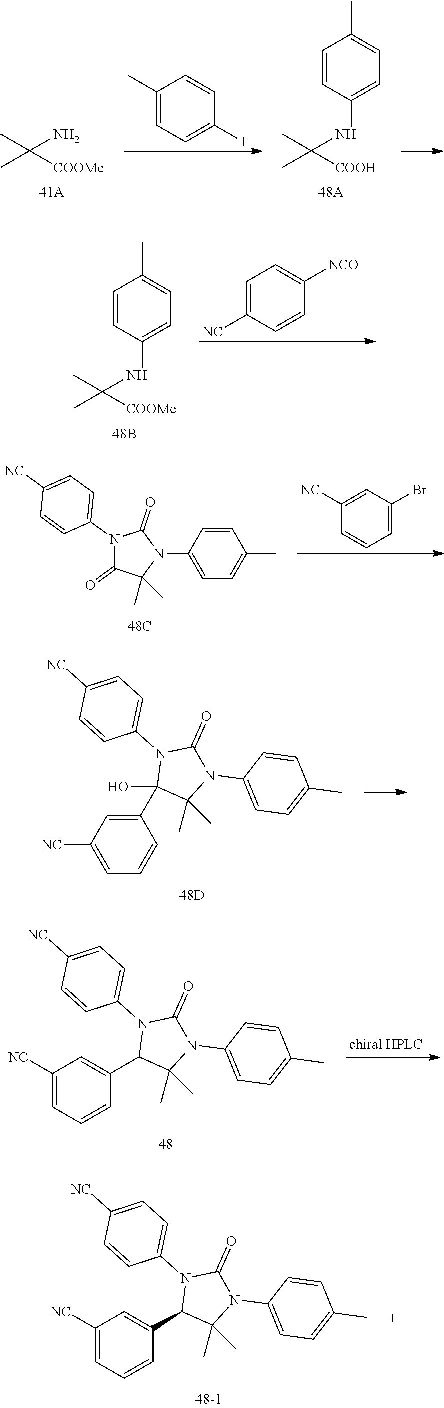

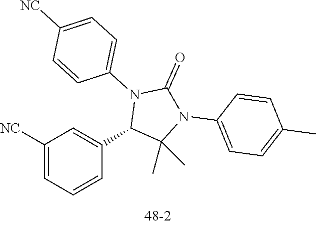 [SMILES: Cc1ccc(N2C(=O)N(c3ccc(C#N)cc3)[C@@H](c3cccc(C#N)c3)C2(C)C)cc1]

Compounds 48A, 48B, 48C, 48D, and 48 were synthesized by employing the procedures described for Compounds 12B, 12C, 14D, 6D, and 6 using 1-iodo-4-methylbenzene, Compounds 41A, 48A, 48B, 4-isocyanatobenzonitrile at 80° C., 48C, 3-bromobenzonitrile, and 48D in lieu of 1-chloro-4-iodobenzene, Compounds 12A, 12B, 14C, 1-chloro-4-isocyanatobenzene at 100° C., 6C, 1-bromo-3-(trifluoromethyl)benzene, and 6D.

Compound 48A. LC-MS (ESI) m/z: 194 [M+H]⁺.

Compound 48B. LC-MS (ESI) m/z: 208 [M+H]⁺.

Compound 48C. LC-MS (ESI) m/z: 320 [M+H]⁺; ¹H-NMR (CDCl₃, 400 MHz): δ (ppm) 1.44 (s, 6H), 3.34 (s, 3H), 7.30-7.31 (m, 4H), 7.73-7.75 (m, 2H), 7.99-8.01 (m, 2H).

Compound 48D. LC-MS (ESI) m/z: 423 [M+H]⁺. ¹H-NMR (CDCl₃, 400 MHz): δ (ppm) 0.81 (s, 3H), 1.31 (s, 3H), 2.38 (s, 3H), 4.34 (s, 1H), 7.09-7.11 (m, 2H), 7.20-7.22 (m, 2H), 7.43-7.46 (m, 4H), 7.55-7.63 (m, 4H).

Compound 48 was separated with chiral HPLC to give Compound 48-1 and Compound 48-2.

Compound 48-1: LC-MS (ESI) m/z: 407 [M+H]⁺; ¹H-NMR (CDCl₃, 400 MHz): δ (ppm) 0.78 (s, 3H), 1.54 (s, 3H), 2.38 (s, 3H), 5.00 (s, 1H), 7.08-7.10 (m, 2H), 7.23-7.25 (m, 2H), 7.49-7.68 (m, 8H). Chiral separation conditions: MeOH contained 0.2% Methanol Ammonia; IC (100×4.6 mm, 5 μm); retention time: 2.59 minutes.

Compound 48-2: LC-MS (ESI) m/z: 407 [M+H]⁺; ¹H-NMR (CDCl₃, 400 MHz): δ (ppm) 0.78 (s, 3H), 1.54 (s, 3H), 2.38 (s, 3H), 5.00 (s, 1H), 7.08-7.10 (m, 2H), 7.23-7.25 (m, 2H), 7.49-7.66 (m, 8H). Chiral separation conditions: MeOH contained 0.2% Methanol Ammonia; IC (100×4.6 mm, 5 μm); retention time: 3.56 minutes.

### Example 49

### Synthesis of 7-(4-chlorophenyl)-5-(p-tolyl)-8-(3-(trifluoromethoxy)phenyl)-2-oxa-5,7-diazaspiro[3.4]octan-6-one (49), (R)-7-(4-chlorophenyl)-5-(p-tolyl)-8-(3-(trifluoromethoxy)phenyl)-2-oxa-5,7-diazaspiro[3.4]octan-6-one (49-1), and (S)-7-(4-chlorophenyl)-5-(p-tolyl)-8-(3-(trifluoromethoxy)phenyl)-2-oxa-5,7-diazaspiro[3.4]octan-6-one (49-2)

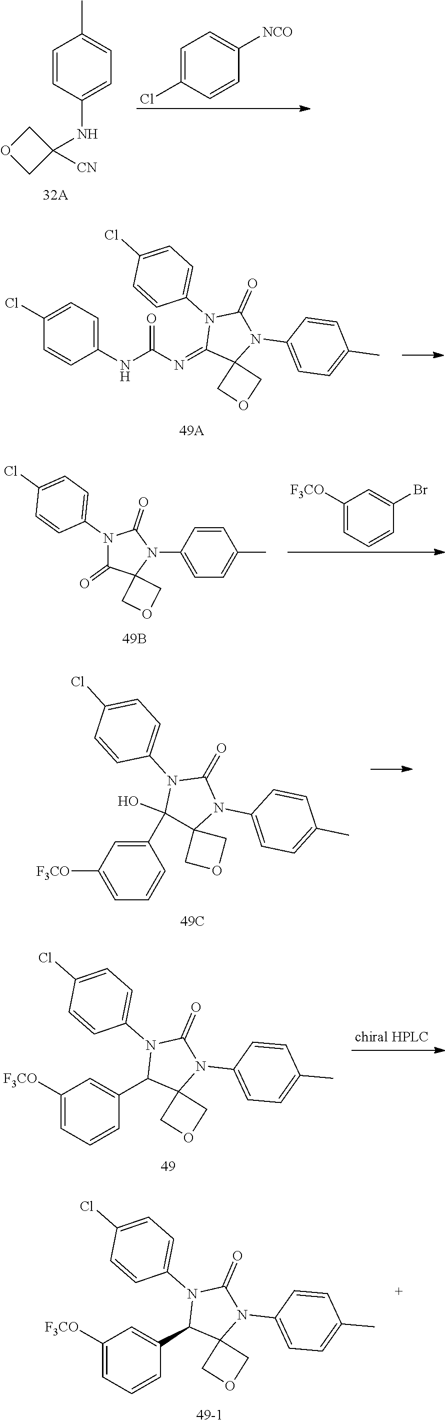

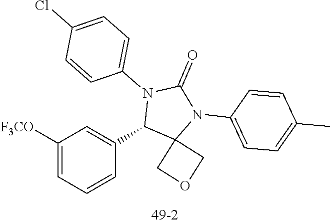 [SMILES: Cc1ccc(N2C(=O)N(c3ccc(Cl)cc3)[C@@H](c3cccc(OC(F)(F)F)c3)C23COC3)cc1]

Compounds 49A, 49B, 49C, and 49 were synthesized by employing the procedures described for Compounds 32B, 32C, 6D, and 29 using 1-chloro-4-isocyanatobenzene, Compounds 49A, 49B, 1-bromo-3-(trifluoromethoxy)benzene, and 49C in lieu of 1-isocyanato-4-methylbenzene, Compounds 32B, 6C, 1-bromo-3-(trifluoromethyl)benzene, and 29F.

Compound 49A. LC-MS (ESI) m/z: 495 [M+H]⁺; ¹H-NMR (DMSO-d₆, 400 MHz): δ (ppm) 2.40 (s, 3H), 4.78 (d, J=7.6 Hz, 2H), 5.06 (d, J=7.6 Hz, 2H), 7.28-7.34 (m, 5H), 7.37-7.40 (m, 5H), 7.47-7.51 (m, 2H), 9.73 (s, 1H).

Compound 49B. LC-MS (ESI) m/z: 343 [M+H]⁺; ¹H-NMR (DMSO-d₆, 400 MHz): δ (ppm) 2.39 (s, 3H), 4.67 (d, J=8.0 Hz, 2H), 4.84 (d, J=8.0 Hz, 2H), 7.38 (d, J=8.0 Hz, 2H), 7.46-7.51 (m, 4H), 7.59 (d, J=8.8 Hz, 2H).

Compound 49C. LC-MS (ESI) m/z: 505 [M+H]⁺; ¹H-NMR (CDCl₃, 400 MHz): δ (ppm) 2.43 (s, 3H), 3.99 (d, J=7.6 Hz, 1H), 4.49 (d, J=7.6 Hz, 1H), 4.68 (d, J=8.0 Hz, 1H), 4.78 (s, 1H), 5.15 (d, J=8.4 Hz, 1H), 7.15-7.18 (m, 4H), 7.22 (d, J=8.0 Hz, 1H), 7.26 (d, J=8.0 Hz, 2H), 7.30 (d, J=9.2 Hz, 2H), 7.36 (s, 1H), 7.39-7.41 (m, 2H).

Compound 49 was separated with chiral HPLC to yield Compound 49-1 and Compound 49-2.

Compound 49-1: LC-MS (ESI) m/z: 489 [M+H]⁺; ¹H-NMR (CDCl₃, 400 MHz): δ (ppm) 2.43 (s, 3H), 4.10 (d, J=7.6 Hz, 1H), 4.54 (d, J=7.6 Hz, 1H), 4.80 (d, J=7.2 Hz, 1H), 5.06 (d, J=7.2 Hz, 1H), 5.47 (s, 1H), 7.20-7.27 (m, 6H), 7.33 (d, J=8.0 Hz, 2H), 7.37 (d, J=8.0 Hz, 1H), 7.43 (d, J=9.2 Hz, 2H), 7.49 (t, J=7.6 Hz, 1H). Chiral separation conditions: MeOH contained 0.2% Methanol Ammonia; OD-H (4.6×100 mm, 5 μm); retention time: 0.97 minutes.

Compound 49-2: LC-MS (ESI) m/z: 489 [M+H]⁺; ¹H-NMR (CDCl₃, 400 MHz): δ (ppm) 2.43 (s, 3H), 4.10 (d, J=7.6 Hz, 1H), 4.54 (d, J=7.6 Hz, 1H), 4.80 (d, J=7.2 Hz, 1H), 5.06 (d, J=7.2 Hz, 1H), 5.47 (s, 1H), 7.20-7.27 (m, 6H), 7.33 (d, J=8.0 Hz, 2H), 7.37 (d, J=8.0 Hz, 1H), 7.43 (d, J=9.2 Hz, 2H), 7.49 (t, J=7.6 Hz, 1H). Chiral separation conditions: MeOH contained 0.2% Methanol Ammonia; OD-H (4.6×100 mm, 5 μm); retention time: 1.98 minutes.

### Example 50

### Synthesis of 4-(4,4-dimethyl-2-oxo-3-(p-tolyl)-5-(3-(trifluoromethoxy) phenyl)imidazolidin-1-yl)benzonitrile (50), (R)-4-(4,4-dimethyl-2-oxo-3-(p-tolyl)-5-(3-(trifluoromethoxy)phenyl)imidazolidin-1-yl)benzonitrile (50-1), and (S)-4-(4,4-dimethyl-2-oxo-3-(p-tolyl)-5-(3-(trifluoromethoxy)phenyl)imidazolidin-1-yl)benzonitrile (50-2)

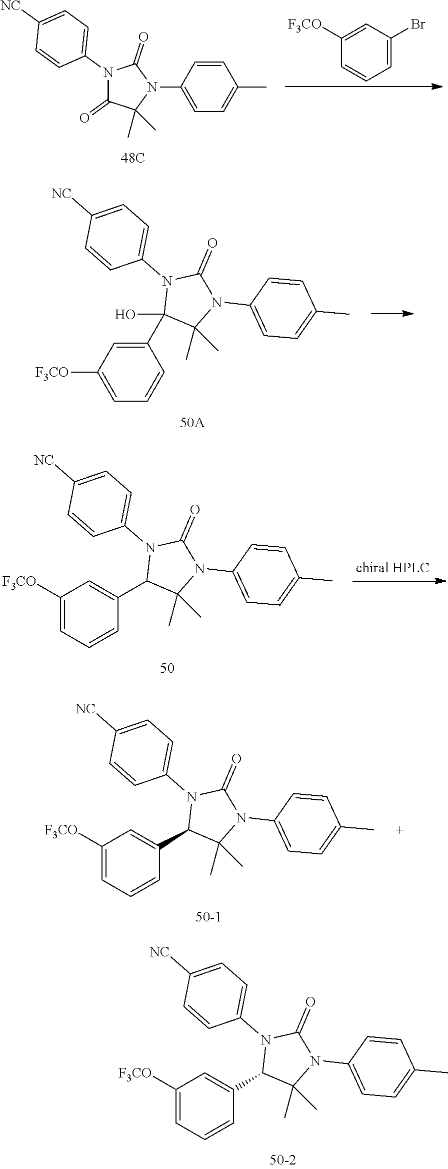 [SMILES: Cc1ccc(N2C(=O)N(c3ccc(C#N)cc3)C(=O)C2(C)C)cc1 ; FC(F)(F)Oc1cccc(Br)c1 ; Cc1ccc(N2C(=O)N(c3ccc(C#N)cc3)C(O)(c3cccc(OC(F)(F)F)c3)C2(C)C)cc1 ; Cc1ccc(N2C(=O)N(c3ccc(C#N)cc3)C(c3cccc(OC(F)(F)F)c3)C2(C)C)cc1 ; Cc1ccc(N2C(=O)N(c3ccc(C#N)cc3)[C@H](c3cccc(OC(F)(F)F)c3)C2(C)C)cc1 ; Cc1ccc(N2C(=O)N(c3ccc(C#N)cc3)[C@@H](c3cccc(OC(F)(F)F)c3)C2(C)C)cc1]

Compounds 50A and 50 were synthesized by employing the procedures described for Compounds 6D and 6 using 1-bromo-3-(trifluoromethoxy)benzene, Compounds 48C at −60° C., and 50A in lieu of 1-bromo-3-(trifluoromethyl)benzene, Compounds 6C at −78° C., and 6D.

Compound 50A. LC-MS (ESI) m/z: 482 [M+H]⁺. ¹H-NMR (CDCl₃, 400 MHz): δ (ppm) 0.80 (s, 3H), 1.29 (s, 3H), 2.37 (s, 3H), 4.55 (s, 1H), 7.07-7.09 (m, 2H), 7.15-7.22 (m, 4H), 7.30-7.35 (m, 2H), 7.42-7.45 (m, 2H), 7.57-7.59 (m, 2H).

Compound 50 was separated with chiral HPLC to give Compound 50-1 and Compound 50-2.

Compound 50-1: LC-MS (ESI) m/z: 466 [M+H]⁺; ¹H-NMR (CDCl₃, 400 MHz): δ (ppm) 0.78 (s, 3H), 1.53 (s, 3H), 2.38 (s, 3H), 4.97 (s, 1H), 7.08-7.14 (m, 3H), 7.20-7.38 (m, 5H), 7.43-7.50 (m, 2H), 7.75-7.60 (m, 2H). Chiral separation conditions: MeOH contained 0.2% Methanol Ammonia; S,S-Whelk-O 1 (100×4.6 mm, 5 μm); retention time: 3.23 minutes.

Compound 50-2: LC-MS (ESI) m/z: 466 [M+H]⁺; ¹H-NMR (CDCl₃, 400 MHz): δ (ppm) 0.79 (s, 3H), 1.53 (s, 3H), 2.38 (s, 3H), 4.97 (s, 1H), 7.08-7.10 (m, 4H), 7.21-7.26 (m, 4H), 7.48-7.60 (m, 4H). Chiral separation conditions: MeOH contained 0.2% Methanol Ammonia; S,S-Whelk-01 (100×4.6 mm, 5 μm); retention time: 1.48 minutes.

### Example 51

### Synthesis of 4,4′-((4R)-4-ethyl-4-methyl-2-oxo-5-(3-(trifluoromethoxy) phenyl)imidazolidine-1,3-diyl)dibenzonitrile (51-1), 4,4′-((4R,5S)-4-ethyl-4-methyl-2-oxo-5-(3-(trifluoromethoxy)phenyl)imidazolidine-1,3-diyl)dibenzonitrile (51-2), 4,4′-((4R,5R)-4-ethyl-4-methyl-2-oxo-5-(3-(trifluoromethoxy)phenyl)imidazolidine-1,3-diyl)dibenzonitrile (51-3), 4,4′-((4S)-4-ethyl-4-methyl-2-oxo-5-(3-(trifluoromethoxy)phenyl)imidazolidine-1,3-diyl)dibenzonitrile (51-4), 4,4′-((4S,5S)-4-ethyl-4-methyl-2-oxo-5-(3-(trifluoromethoxy) phenyl)imidazolidine-1,3-diyl)dibenzonitrile (51-5), and 4,4′-((4S,5R)-4-ethyl-4-methyl-2-oxo-5-(3-(trifluoromethoxy)phenyl)imidazolidine-1,3-diyl)dibenzonitrile (51-6)

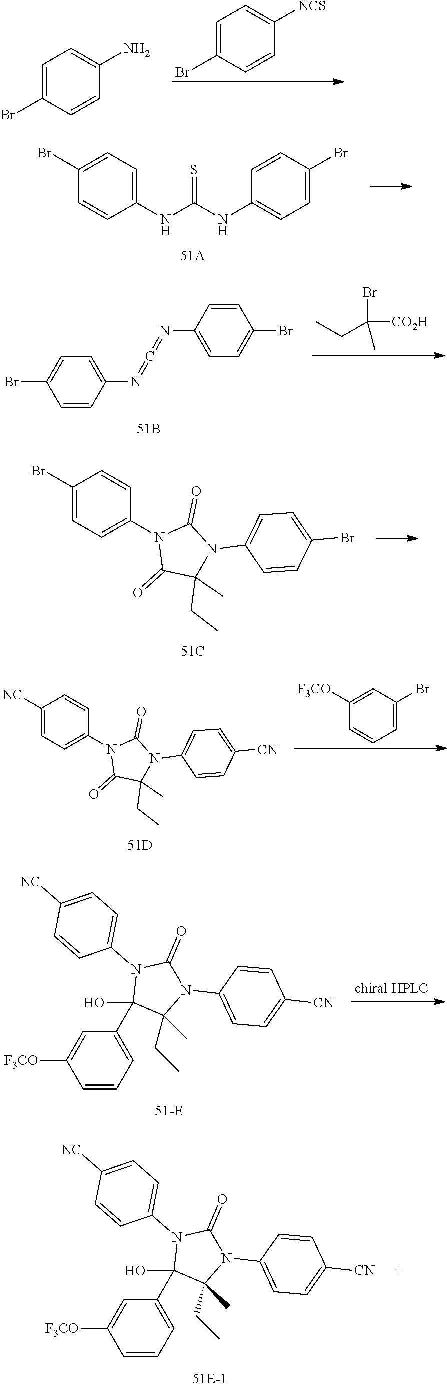

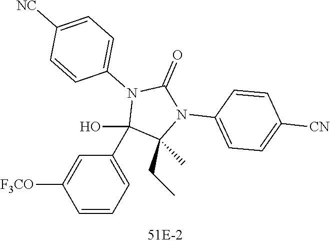 [SMILES: CC[C@]1(C)N(c2ccc(C#N)cc2)C(=O)N(c2ccc(C#N)cc2)C1(O)c1cccc(OC(F)(F)F)c1]

Compounds 51A, 51B, 51C, 51D, and 51E were synthesized by employing the procedures described for Compounds 6A, 6B, 6C, 33B, and 6D using 1-bromo-4-isocyanatobenzene, 4-bromoaniline, Compounds 51A, 51B, 2-bromo-2-methylbutanoic acid, 51C, 1-bromo-3-(trifluoromethoxy)benzene, and 51D at −60° C. in lieu of 1-isocyanato-4-methylbenzene, 4-chloroaniline, Compounds 6A, 6B, 2-bromo-2-methylpropanoic acid, 33A, 1-bromo-3-(trifluoromethyl)benzene, and 6C at −78° C.

Compound 51A. LC-MS (ESI) m/z: 385 [M+H]⁺.

Compound 51B. ¹H-NMR (CDCl₃, 400 MHz): δ (ppm) 7.03 (m, 4H), 7.44 (m, 4H).

Compound 51C. LC-MS (ESI) m/z: 451 [M+H]⁺; ¹H-NMR: (CDCl₃, 400 MHz): δ (ppm) 1.00 (t, J=7.2 Hz, 3H), 1.54 (s, 3H), 1.69-1.75 (m, 1H), 1.99-2.04 (m, 1H), 7.20-7.23 (m, 2H), 7.35-7.39 (m, 2H), 7.57-7.62 (m, 4H).

Compound 51D. MS (ESI) m/z: 345 [M+H]⁺.

Compound 51E was separated with chiral HPLC to give Compound 51E-1 and Compound 51E-2.

Compound 51E-1: LC-MS (ESI) m/z: 507 [M+H]⁺; ¹H-NMR (CDCl₃, 400 MHz): δ (ppm) 0.89 (t, J=7.2 Hz, 3H), 0.97 (s, 3H), 1.82-1.87 (m, 1H), 1.99-2.07 (m, 1H), 4.24 (s, 1H), 7.20-7.22 (m, 1H), 7.37-7.54 (m, 9H), 7.67-7.71 (m, 2H). Chiral separation conditions: MeOH contained 0.2% Methanol Ammonia; OD-H (4.6×100 mm, 5 μm); retention time: 1.31 minutes.

Compound 51E-2: LC-MS (ESI) m/z: 507 [M+H]⁺; ¹H-NMR (CDCl₃, 400 MHz): δ (ppm) 0.89 (t, J=7.2 Hz, 3H), 0.97 (s, 3H), 1.84-1.88 (m, 1H), 1.99-2.06 (m, 1H), 4.20 (s, 1H), 7.20-7.22 (m, 1H), 7.36-7.47 (m, 9H), 7.68-7.72 (m, 2H). Chiral separation conditions: MeOH contained 0.2% Methanol Ammonia; OD-H (4.6×100 mm, 5 μm); retention time: 2.22 minutes.

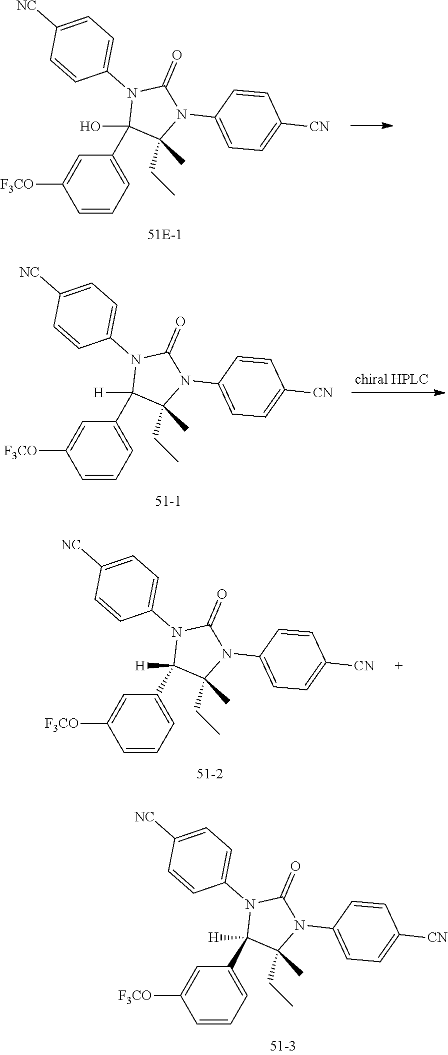 [SMILES: CC[C@@]1(C)N(c2ccc(C#N)cc2)C(=O)N(c2ccc(C#N)cc2)C1(O)c1cccc(OC(F)(F)F)c1 ; CC[C@]1(C)C(c2cccc(OC(F)(F)F)c2)N(c2ccc(C#N)cc2)C(=O)N1c1ccc(C#N)cc1 ; CC[C@]1(C)[C@H](c2cccc(OC(F)(F)F)c2)N(c2ccc(C#N)cc2)C(=O)N1c1ccc(C#N)cc1 ; CC[C@]1(C)[C@@H](c2cccc(OC(F)(F)F)c2)N(c2ccc(C#N)cc2)C(=O)N1c1ccc(C#N)cc1]

Compound 51-1 was synthesized by employing the procedure described for Compound 6 using Compound 51E-1 at room temperature in lieu of Compound 6D at 10° C., which was separated with chiral HPLC to give Compound 51-2 and Compound 51-3.

Compound 51-2: LC-MS (ESI) m/z: 491 [M+H]⁺; ¹H-NMR (CDCl₃, 400 MHz): δ (ppm) 0.59 (t, J=7.2 Hz, 3H), 1.33-1.41 (m, 2H), 1.61 (s, 3H), 4.98 (s, 1H), 7.23-7.25 (m, 2H), 7.29-7.31 (m, 1H), 7.38-7.41 (m, 2H), 7.43-7.47 (m, 1H), 7.51-7.58 (m, 4H), 7.73-7.75 (m, 2H). Chiral separation conditions: MeOH contained 0.2% Methanol Ammonia; S,S-Whelk-Ol (100×4.6 mm, 5 μm); retention time: 3.97 minutes.

Compound 51-3: LC-MS (ESI) m/z: 491 [M+H]⁺; ¹H-NMR (CDCl₃, 400 MHz): δ (ppm) 0.82 (s, 3H), 1.10 (t, J=7.2 Hz, 3H), 1.80-1.84 (m, 1H), 1.93-1.96 (m, 1H), 5.00 (s, 1H), 7.03-7.24 (m, 3H), 7.36-7.39 (m, 2H), 7.45-7.48 (m, 1H), 7.52-7.54 (m, 2H), 7.60-7.62 (m, 2H), 7.71-7.73 (m, 2H). Chiral separation conditions: MeOH contained 0.2% Methanol Ammonia; S,S-Whelk-Ol (100×4.6 mm, 5 μm); retention time: 2.05 minutes.

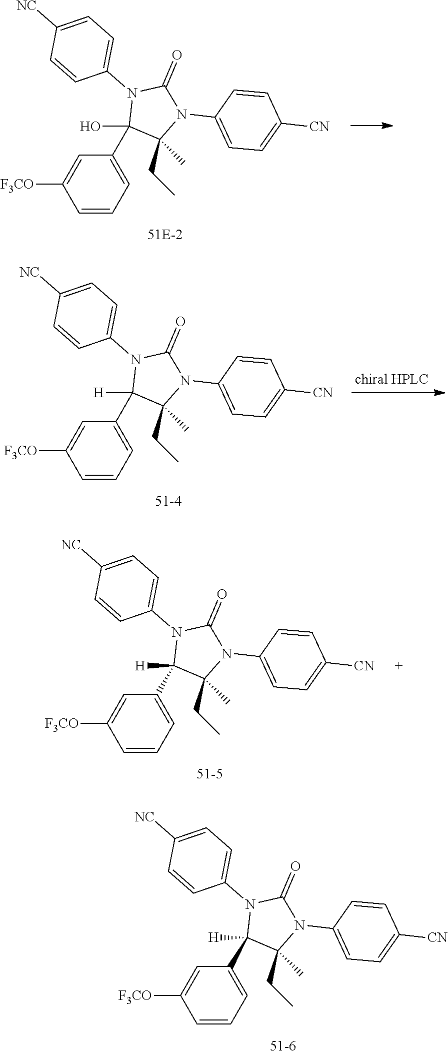 [SMILES: CC[C@]1(C)N(c2ccc(C#N)cc2)C(=O)N(c2ccc(C#N)cc2)C1(O)c1cccc(OC(F)(F)F)c1 ; CC[C@@]1(C)C(c2cccc(OC(F)(F)F)c2)N(c2ccc(C#N)cc2)C(=O)N1c1ccc(C#N)cc1 ; CC[C@@]1(C)[C@H](c2cccc(OC(F)(F)F)c2)N(c2ccc(C#N)cc2)C(=O)N1c1ccc(C#N)cc1 ; CC[C@@]1(C)[C@@H](c2cccc(OC(F)(F)F)c2)N(c2ccc(C#N)cc2)C(=O)N1c1ccc(C#N)cc1]

Compound 51-4 was synthesized by employing the procedure described for Compound 6 using Compound 51E-2 at room temperature in lieu of Compound 6D at 10° C., which was further purified with chiral HPLC to give Compound 51-5 and Compound 51-6.

Compound 51-5: LC-MS (ESI) m/z: 491 [M+H]⁺; ¹H-NMR (CDCl₃, 400 MHz): δ (ppm) 0.82 (s, 3H), 1.10 (t, J=7.2 Hz, 3H), 1.78-1.84 (m, 1H), 1.91-1.98 (m, 1H), 5.00 (s, 1H), 7.03-7.23 (m, 3H), 7.37-7.39 (m, 2H), 7.45-7.48 (m, 1H), 7.52-7.54 (m, 2H), 7.60-7.62 (m, 2H), 7.71-7.73 (m, 2H). Chiral separation conditions: MeOH contained 0.2% Methanol Ammonia; S,S-Whelk-Ol (100×4.6 mm, 5 μm); retention time: 2.93 minutes.

Compound 51-6: LC-MS (ESI) m/z: 491 [M+H]⁺; ¹H-NMR (CDCl₃, 400 MHz): δ (ppm) 0.59 (t, J=7.2 Hz, 3H), 1.35-1.39 (m, 2H), 1.61 (s, 3H), 4.98 (s, 1H), 7.23-7.25 (m, 2H), 7.29-7.31 (m, 1H), 7.39-7.45 (m, 3H), 7.51-7.58 (m, 4H), 7.73-7.75 (m, 2H). Chiral separation conditions: MeOH contained 0.2% Methanol Ammonia; S,S-Whelk-Ol (100×4.6 mm, 5 μm); retention time: 2.23 minutes.

### Example 52

### Synthesis of 4,4′-((4R)-4-methyl-2-oxo-4-propyl-5-(3-(trifluoromethoxy) phenyl)imidazolidine-1,3-diyl)dibenzonitrile (52-1), 4,4′-((4R,5S)-4-methyl-2-oxo-4-propyl-5-(3-(trifluoromethoxy)phenyl)imidazolidine-1,3-diyl)dibenzonitrile (52-2), 4,4′-((4R,5R)-4-methyl-2-oxo-4-propyl-5-(3-(trifluoromethoxy)phenyl) imidazolidine-1,3-diyl)dibenzonitrile (52-3), 4,4′-((4S)-4-methyl-2-oxo-4-propyl-5-(3-(trifluoromethoxy) phenyl)imidazolidine-1,3-diyl)dibenzonitrile (52-4), 4,4′-((4S,5S)-4-methyl-2-oxo-4-propyl-5-(3-(trifluoromethoxy)phenyl)imidazolidine-1,3-diyl)dibenzonitrile (52-5), and 4,4′-((4S,5R)-4-methyl-2-oxo-4-propyl-5-(3-(trifluoromethoxy)phenyl)imidazolidine-1,3-diyl)dibenzonitrile (52-6)

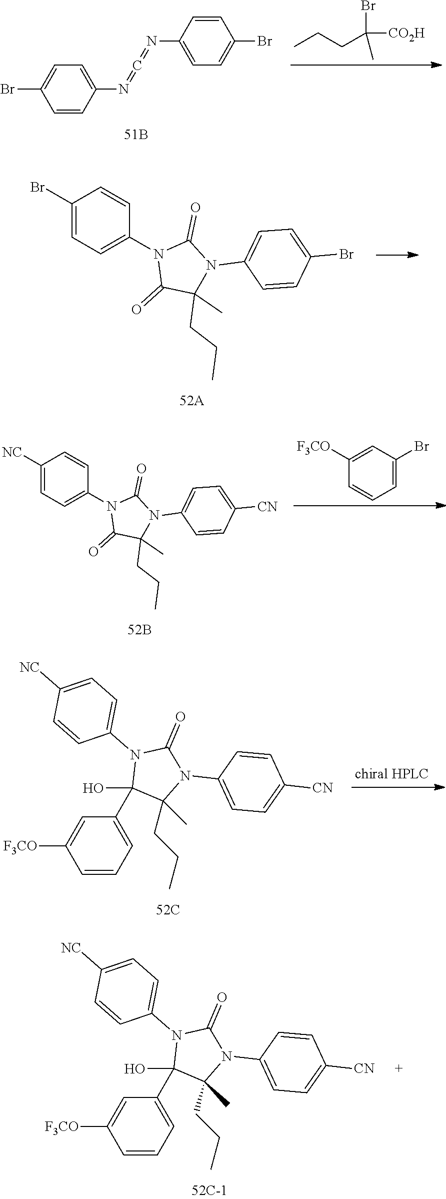

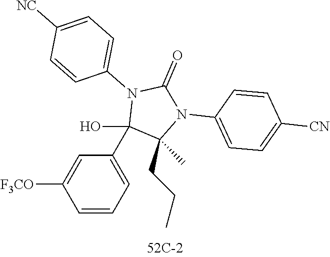 [SMILES: CCC[C@]1(C)N(c2ccc(C#N)cc2)C(=O)N(c2ccc(C#N)cc2)C1(O)c1cccc(OC(F)(F)F)c1]

Compounds 52A, 52B, and 52C were synthesized by employing the procedures described for Compounds 6C, 33B, and 6D using 2-bromo-2-methylpentanoic acid, Compounds 51B, 52A, 1-bromo-3-(trifluoromethoxy)benzene, and 52B at −60° C. in lieu of 2-bromo-2-methylpropanoic acid, Compounds 6B, 33A, 1-bromo-3-(trifluoromethyl)benzene, and 6C at −78° C.

Compound 52A. LC-MS (ESI) m/z: 465 [M+H]⁺; ¹H-NMR: (CDCl₃, 400 MHz): δ (ppm) 0.94 (t, J=7.2 Hz, 3H), 1.30-1.33 (m, 1H), 1.48-1.51 (m, 1H), 1.53 (s, 3H), 1.62-1.64 (m, 1H), 1.90-1.95 (m, 1H), 7.18-7.21 (m, 2H), 7.35-7.38 (m, 2H), 7.58-7.61 (m, 4H).

Compound 52B. MS (ESI) m/z: 359 [M+H]⁺.

Compound 52C was separated with chiral HPLC to give Compound 52C-1 and Compound 52C-2.

Compound 52C-1: LC-MS (ESI) m/z: 521 [M+H]⁺; ¹H-NMR (CDCl₃, 400 MHz): δ (ppm) 0.87 (t, J=7.2 Hz, 3H), 0.97 (s, 3H), 1.04-1.12 (m, 1H), 1.39-1.48 (m, 1H), 1.72-1.79 (m, 1H), 1.89-2.00 (m, 1H), 3.97 (s, 1H), 7.21-7.24 (m, 1H), 7.33-7.48 (m, 9H), 7.70-7.74 (m, 2H). Chiral separation conditions: MeOH contained 0.2% Methanol Ammonia; OD-H (4.6×100 mm, 5 μm); retention time: 1.02 minutes.

Compound 52C-2: LC-MS (ESI) m/z: 521 [M+H]⁺; ¹H-NMR (CDCl₃, 400 MHz): δ (ppm) 0.88 (t, J=7.2 Hz, 3H), 0.97 (s, 3H), 1.06-1.10 (m, 1H), 1.40-1.46 (m, 1H), 1.71-1.78 (m, 1H), 1.89-1.94 (m, 1H), 4.06 (s, 1H), 7.20-7.22 (m, 1H), 7.33-7.54 (m, 9H), 7.69-7.73 (m, 2H). Chiral separation conditions: MeOH contained 0.2% Methanol Ammonia; OD-H (4.6×100 mm, 5 μm); retention time: 1.65 minutes.

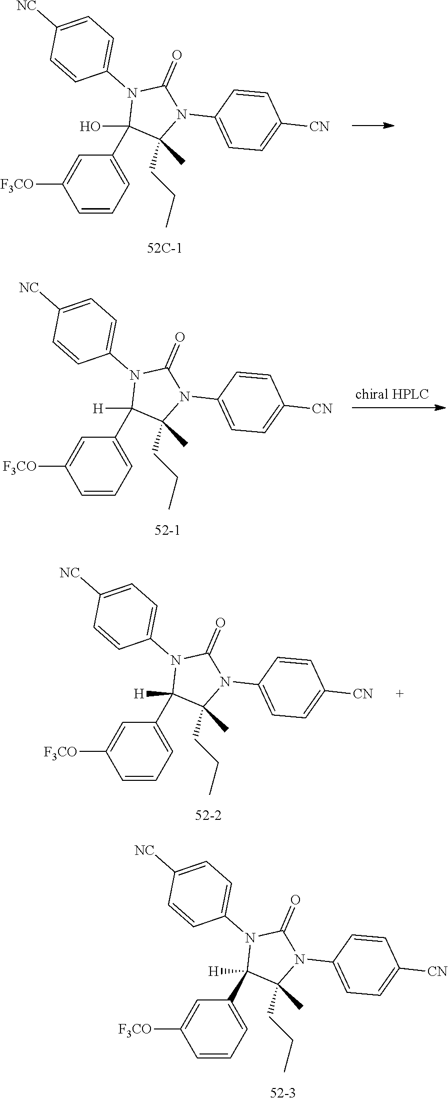 [SMILES: CCC[C@@]1(C)N(c2ccc(C#N)cc2)C(=O)N(c2ccc(C#N)cc2)C1(O)c1cccc(OC(F)(F)F)c1 ; CCC[C@]1(C)C(c2cccc(OC(F)(F)F)c2)N(c2ccc(C#N)cc2)C(=O)N1c1ccc(C#N)cc1 ; CCC[C@]1(C)[C@H](c2cccc(OC(F)(F)F)c2)N(c2ccc(C#N)cc2)C(=O)N1c1ccc(C#N)cc1 ; CCC[C@]1(C)[C@@H](c2cccc(OC(F)(F)F)c2)N(c2ccc(C#N)cc2)C(=O)N1c1ccc(C#N)cc1]

Compound 52-1 was synthesized by employing the procedure described for Compound 6 using Compound 52C-1 at room temperature in lieu of Compound 6D at 10° C., which was separated with chiral HPLC to give Compound 52-2 and Compound 52-3.

Compound 52-2: LC-MS (ESI) m/z: 505 [M+H]⁺; ¹H-NMR (CDCl₃, 400 MHz): δ (ppm) 0.52 (t, J=7.2 Hz, 3H), 0.95-0.97 (m, 1H), 1.06-1.15 (m, 2H), 1.30-1.33 (m, 1H), 1.60 (s, 3H), 4.97 (s, 1H), 7.19-7.24 (m, 2H), 7.29-7.31 (m, 1H), 7.37-7.41 (m, 2H), 7.37-7.47 (m, 1H), 7.49-7.59 (m, 4H), 7.73-7.76 (m, 2H). Chiral separation conditions: MeOH contained 0.2% Methanol Ammonia; S,S-Whelk-Ol (100×4.6 mm, 5 μm); retention time: 3.88 minutes.

Compound 52-3: LC-MS (ESI) m/z: 505 [M+H]⁺; ¹H-NMR (CDCl₃, 400 MHz): δ (ppm) 0.52 (t, J=7.2 Hz, 3H), 0.85-0.88 (m, 1H), 1.07-1.11 (m, 2H), 1.30-1.33 (m, 1H), 1.60 (s, 3H), 4.97 (s, 1H), 7.15-7.24 (m, 2H), 7.29-7.31 (m, 1H), 7.38-7.47 (m, 3H), 7.51-7.59 (m, 4H), 7.71-7.75 (m, 2H). Chiral separation conditions: MeOH contained 0.2% Methanol Ammonia; S,S-Whelk-Ol (100×4.6 mm, 5 μm); retention time: 2.21 minutes.

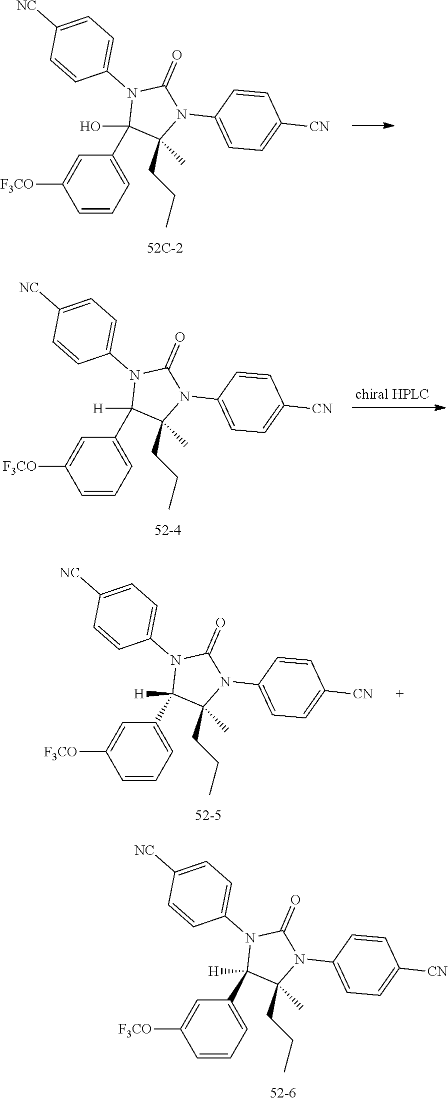 [SMILES: CCC[C@]1(C)N(c2ccc(C#N)cc2)C(=O)N(c2ccc(C#N)cc2)C1(O)c1cccc(OC(F)(F)F)c1 ; CCC[C@@]1(C)C(c2cccc(OC(F)(F)F)c2)N(c2ccc(C#N)cc2)C(=O)N1c1ccc(C#N)cc1 ; CCC[C@@]1(C)[C@H](c2cccc(OC(F)(F)F)c2)N(c2ccc(C#N)cc2)C(=O)N1c1ccc(C#N)cc1 ; CCC[C@@]1(C)[C@@H](c2cccc(OC(F)(F)F)c2)N(c2ccc(C#N)cc2)C(=O)N1c1ccc(C#N)cc1]

Compound 52-4 was synthesized by employing the procedure described for Compound 6 using Compound 52C-2 at room temperature in lieu of Compound 6D at 10° C., which was separated with chiral HPLC to give Compound 52-5 and Compound 52-6.

Compound 52-5: LC-MS (ESI) m/z: 505 [M+H]⁺; ¹H-NMR (CDCl₃, 400 MHz): δ (ppm) 0.52 (t, J=7.2 Hz, 3H), 0.95-0.97 (m, 1H), 1.06-1.15 (m, 2H), 1.33-1.36 (m, 1H), 1.60 (s, 3H), 4.97 (s, 1H), 7.19-7.24 (m, 2H), 7.29-7.31 (m, 1H), 7.38-7.40 (m, 2H), 7.43-7.45 (m, 1H), 7.51-7.53 (m, 2H), 7.56-7.59 (m, 2H), 7.73-7.75 (m, 2H). Chiral separation conditions: MeOH contained 0.2% Methanol Ammonia; S,S-Whelk-Ol (100×4.6 mm, 5 μm); retention time: 3.96 minutes.

Compound 52-6: LC-MS (ESI) m/z: 505 [M+H]⁺; ¹H-NMR (CDCl₃, 400 MHz): δ (ppm) 0.52 (t, J=7.2 Hz, 3H), 0.95-0.99 (m, 1H), 1.14-1.18 (m, 2H), 1.32-1.37 (m, 1H), 1.60 (s, 3H), 4.97 (s, 1H), 7.15-7.24 (m, 2H), 7.28-7.31 (m, 1H), 7.37-7.41 (m, 2H), 7.43-7.47 (m, 1H), 7.50-7.59 (m, 4H), 7.73-7.76 (m, 2H). Chiral separation conditions: MeOH contained 0.2% Methanol Ammonia; S,S-Whelk-Ol (100×4.6 mm, 5 μm); retention time: 2.14 minutes.

### Example 53

### Synthesis of 4-(5,5-dimethyl-2-oxo-3-(p-tolyl)-4-(3-(trifluoromethoxy)phenyl) imidazolidin-1-yl)benzonitrile (53), (R)-4-(5,5-dimethyl-2-oxo-3-(p-tolyl)-4-(3-(trifluoromethoxy)phenyl)imidazolidin-1-yl)benzonitrile (53-1), and (S)-4-(5,5-dimethyl-2-oxo-3-(p-tolyl)-4-(3-(trifluoromethoxy)phenyl)imidazolidin-1-yl)benzonitrile (53-2)

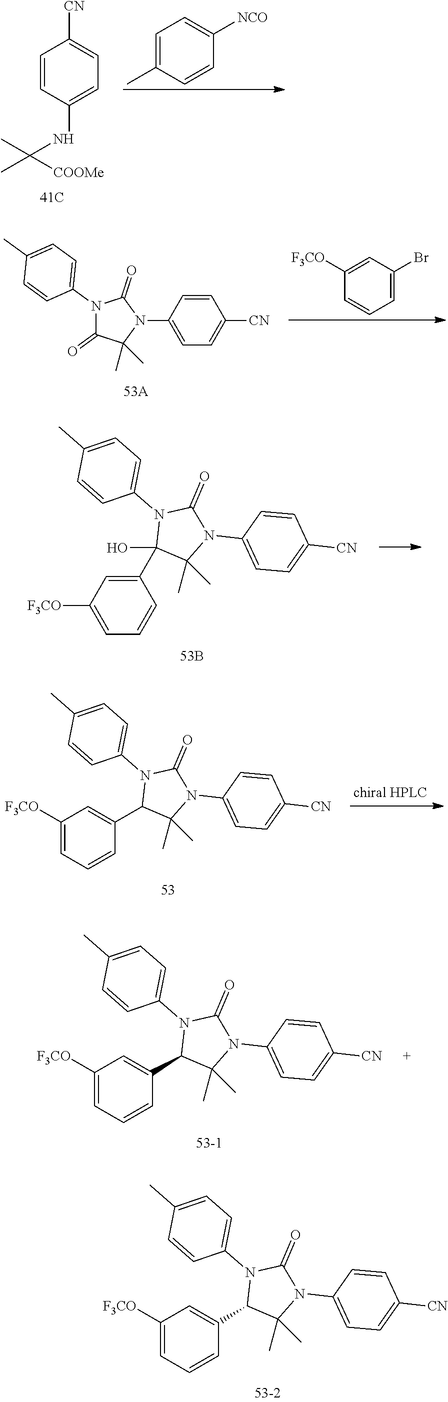

Compounds 53A, 53B, and 53 were synthesized by employing the procedures described for Compounds 14D, 6D, and 6 using 1-isocyanato-4-methylbenzene, Compounds 41C, 53A, 1-bromo-3-(trifluoromethoxy)benzene, and 53B at room temperature in lieu of 1-chloro-4-isocyanatobenzene, Compounds 14C, 6C, 1-bromo-3-(trifluoromethyl)benzene, and 6D at 10° C.

Compound 53A. LC-MS (ESI) m/z: 320 [M+H]⁺; ¹H-NMR (CDCl₃, 400 MHz): δ (ppm) 1.62 (s, 6H), 2.40 (s, 3H), 7.28-7.34 (m, 4H), 7.54-7.55 (m, 2H), 7.75-7.77 (m, 2H).

Compound 53B. LC-MS (ESI) m/z: 482 [M+H]⁺; ¹H-NMR (CDCl₃, 400 MHz): δ (ppm) 0.83 (s, 3H), 1.37 (s, 3H), 2.28 (s, 3H), 4.17 (s, 1H), 7.04 (d, J=6.8 Hz, 2H), 7.15 (d, J=6.4 Hz, 1H), 7.26-7.37 (m, 7H), 7.60 (d, J=6.8 Hz, 2H).

Compound 53. LC-MS (ESI) m/z: 466 [M+H]⁺. Compound 53 was separated with chiral HPLC to give Compound 53-1 and Compound 53-2.

Compound 53-1: LC-MS (ESI) m/z: 466 [M+H]⁺; ¹H-NMR (CDCl₃, 400 MHz): δ (ppm) 0.87 (s, 3H), 1.59 (s, 3H), 2.26 (s, 3H), 4.97 (s, 1H), 7.06 (d, J=6.8 Hz, 2H), 7.15-7.18 (m, 2H), 7.22-7.29 (m, 3H), 7.37-7.43 (m, 3H), 7.70 (d, J=6.8 Hz, 2H). Chiral separation conditions: MeOH contained 0.2% NH₄OH; OD-H (100×4.6 mm, 5 μm); retention time: 0.87 minutes.

Compound 53-2: LC-MS (ESI) m/z: 466 [M+H]⁺; ¹H-NMR (CDCl₃, 400 MHz): δ (ppm) 0.87 (s, 3H), 1.59 (s, 3H), 2.26 (s, 3H), 4.97 (s, 1H), 7.06 (d, J=6.8 Hz, 2H), 7.15-7.18 (m, 2H), 7.22-7.29 (m, 3H), 7.37-7.43 (m, 3H), 7.70 (d, J=6.8 Hz, 2H). Chiral separation conditions: MeOH contained 0.2% NH₄OH; OD-H (100×4.6 mm, 5 μm); retention time: 1.84 minutes.

### Example 54

### Synthesis of 3-(1-(4-cyanophenyl)-5,5-dimethyl-2-oxo-3-(p-tolyl) imidazolidin-4-yl)benzonitrile (54), (R)-3-(1-(4-cyanophenyl)-5,5-dimethyl-2-oxo-3-(p-tolyl)imidazolidin-4-yl)benzonitrile (54-1), and (S)-3-(1-(4-cyanophenyl)-5,5-dimethyl-2-oxo-3-(p-tolyl)imidazolidin-4-yl)benzonitrile (54-2)

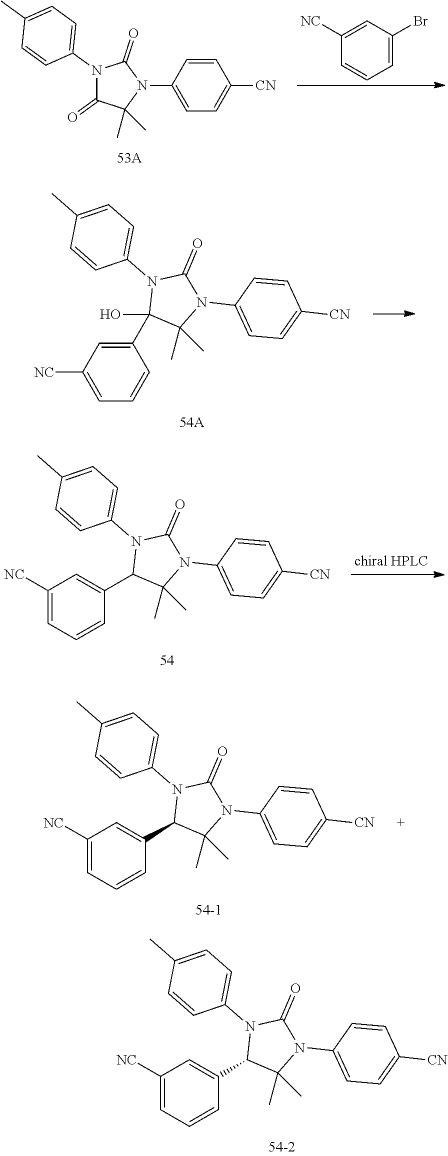 [SMILES: Cc1ccc(N2C(=O)N(c3ccc(C#N)cc3)C(C)(C)C2=O)cc1 ; N#Cc1cccc(Br)c1 ; Cc1ccc(N2C(=O)N(c3ccc(C#N)cc3)C(C)(C)C2(O)c2cccc(C#N)c2)cc1 ; Cc1ccc(N2C(=O)N(c3ccc(C#N)cc3)C(C)(C)C2c2cccc(C#N)c2)cc1 ; Cc1ccc(N2C(=O)N(c3ccc(C#N)cc3)C(C)(C)[C@H]2c2cccc(C#N)c2)cc1 ; Cc1ccc(N2C(=O)N(c3ccc(C#N)cc3)C(C)(C)[C@@H]2c2cccc(C#N)c2)cc1]

Compounds 54A and 54 were synthesized by employing the procedures described for Compounds 6D and 6 using Compounds 53A, 3-bromobenzonitrile, and 54A at room temperature in lieu of Compounds 6C, 1-bromo-3-(trifluoromethyl)benzene, and 6D at 10° C.

Compound 54A. LC-MS (ESI) m/z: 423 [M+H]⁺; ¹H-NMR (CDCl₃, 400 MHz): δ (ppm) 0.83 (s, 3H), 1.36 (s, 3H), 2.29 (s, 3H), 4.42 (s, 1H), 7.04 (d, J=6.8 Hz, 2H), 7.26 (d, J=6.4 Hz, 2H), 7.35-7.40 (m, 3H), 7.57-7.62 (m, 4H), 7.80 (s, 1H).

Compound 54. LC-MS (ESI) m/z: 407 [M+H]⁺. Compound 54 was separated with chiral HPLC to afford Compound 54-1 and Compound 54-2.

Compound 54-1: LC-MS (ESI) m/z: 407 [M+H]⁺. ¹H-NMR (CDCl₃, 400 MHz): δ (ppm) 0.87 (s, 3H), 1.59 (s, 3H), 2.26 (s, 3H), 5.01 (s, 1H), 7.06 (d, J=6.4 Hz, 2H), 7.25 (d, J=6.4 Hz, 2H), 7.43 (d, J=6.8 Hz, 2H), 7.47-7.50 (m, 1H), 7.54-7.55 (m, 1H), 7.59 (s, 1H), 7.62-7.63 (m, 1H), 7.71 (d, J=6.8 Hz, 2H). Chiral separation conditions: MeOH contained 0.2% NH₄OH; EnantioPak OD (100×4.6 mm, 5 μm); retention time: 1.64 minutes.

Compound 54-2: LC-MS (ESI) m/z: 407 [M+H]⁺. ¹H-NMR (CDCl₃, 400 MHz): δ (ppm) 0.87 (s, 3H), 1.59 (s, 3H), 2.26 (s, 3H), 5.01 (s, 1H), 7.06 (d, J=6.4 Hz, 2H), 7.25 (d, J=6.4 Hz, 2H), 7.43 (d, J=6.8 Hz, 2H), 7.47-7.50 (m, 1H), 7.54-7.55 (m, 1H), 7.59 (s, 1H), 7.62-7.63 (m, 1H), 7.71 (d, J=6.8 Hz, 2H). Chiral separation conditions: MeOH contained 0.2% NH₄OH; EnantioPak OD (100×4.6 mm, 5 μm); retention time: 3.16 minutes.

### Example 55

### Synthesis of 3-(7-(4-cyanophenyl)-6-oxo-5-(p-tolyl)-2-oxa-5,7-diazaspiro[3.4]octan-8-yl)benzonitrile (55), (R)-3-(7-(4-cyanophenyl)-6-oxo-5-(p-tolyl)-2-oxa-5,7-diazaspiro[3.4]octan-8-yl)benzonitrile (55-1), and (S)-3-(7-(4-cyanophenyl)-6-oxo-5-(p-tolyl)-2-oxa-5,7-diazaspiro[3.4]octan-8-yl)benzonitrile (55-2)

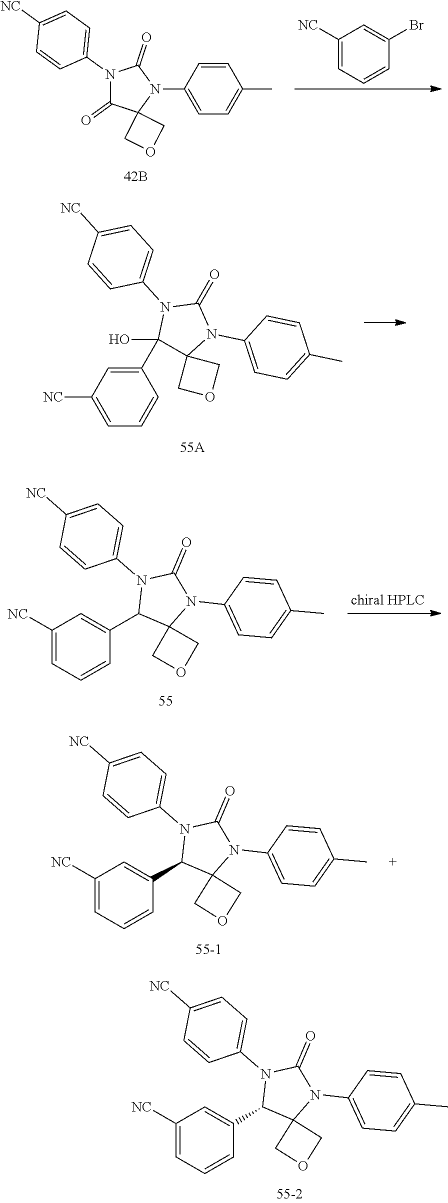 [SMILES: Cc1ccc(N2C(=O)N(c3ccc(C#N)cc3)C(=O)C23COC3)cc1 ; N#Cc1cccc(Br)c1 ; Cc1ccc(N2C(=O)N(c3ccc(C#N)cc3)C(O)(c3cccc(C#N)c3)C23COC3)cc1 ; Cc1ccc(N2C(=O)N(c3ccc(C#N)cc3)C(c3cccc(C#N)c3)C23COC3)cc1 ; Cc1ccc(N2C(=O)N(c3ccc(C#N)cc3)[C@H](c3cccc(C#N)c3)C23COC3)cc1 ; Cc1ccc(N2C(=O)N(c3ccc(C#N)cc3)[C@@H](c3cccc(C#N)c3)C23COC3)cc1]

Compounds 55A and 55 were synthesized by employing the procedures described for Compounds 6D and 6 using Compounds 42B, 3-bromobenzonitrile, and 55A at room temperature in lieu of Compounds 6C, 1-bromo-3-(trifluoromethyl)benzene, and 6D at 10° C.

Compound 55A. LC-MS (ESI) m/z: 437 [M+H]⁺; ¹H-NMR (CDCl₃, 400 MHz): δ (ppm) 2.43 (s, 3H), 3.85 (d, J=7.6 Hz, 1H), 4.50 (d, J=7.6 Hz, 1H), 4.68 (d, J=8.4 Hz, 1H), 5.11 (d, J=7.6 Hz, 1H), 5.40 (s, 1H), 7.19 (d, J=8.0 Hz, 2H), 7.26-7.28 (m, 3H), 7.41 (d, J=8.8 Hz, 2H), 7.47-7.53 (m, 3H), 7.67 (d, J=7.6 Hz, 1H), 7.90 (s, 1H).

Compound 55 was separated with chiral HPLC to yield Compound 55-1 and Compound 55-2.

Compound 55-1: LC-MS (ESI) m/z: 421 [M+H]⁺; ¹H-NMR (CDCl₃, 400 MHz): δ (ppm) 2.45 (s, 3H), 4.03 (d, J=8.0 Hz, 1H), 4.54 (d, J=7.6 Hz, 1H), 4.79 (d, J=7.2 Hz, 1H), 5.07 (d, J=7.2 Hz, 1H), 5.56 (s, 1H), 7.19 (d, J=8.0 Hz, 2H), 7.36 (d, J=8.0 Hz, 2H), 7.56 (d, J=8.8 Hz, 2H), 7.60-7.64 (m, 3H), 7.68 (d, J=8.0 Hz, 1H), 7.74-7.75 (m, 2H). Chiral separation conditions: MeOH contained 0.2% Methanol Ammonia; OZ-H (4.6×100 mm, 5 μm); retention time: 2.51 minutes.

Compound 55-2: LC-MS (ESI) m/z: 421 [M+H]⁺; ¹H-NMR (CDCl₃, 400 MHz): δ (ppm) 2.45 (s, 3H), 4.03 (d, J=8.4 Hz, 1H), 4.55 (d, J=7.6 Hz, 1H), 4.80 (d, J=7.2 Hz, 1H), 5.07 (d, J=7.2 Hz, 1H), 5.56 (s, 1H), 7.19 (d, J=8.0 Hz, 2H), 7.36 (d, J=8.0 Hz, 2H), 7.56 (d, J=8.8 Hz, 2H), 7.60-7.64 (m, 3H), 7.69 (d, J=8.0 Hz, 1H), 7.74-7.75 (m, 2H). Chiral separation conditions: MeOH contained 0.2% Methanol Ammonia; OZ-H (4.6×100 mm, 5 μm); retention time: 1.98 minutes.

### Example 56

### Synthesis of 1-(4-chlorophenyl)-4,4-dimethyl-3-(p-tolyl)-5-(3-(trifluoromethoxy)phenyl)imidazolidin-2-one (56), (R)-1-(4-chlorophenyl)-4,4-dimethyl-3-(p-tolyl)-5-(3-(trifluoromethoxy) phenyl)imidazolidin-2-one (56-1), and (S)-1-(4-chlorophenyl)-4,4-dimethyl-3-(p-tolyl)-5-(3-(trifluoromethoxy)phenyl)imidazolidin-2-one (56-2)

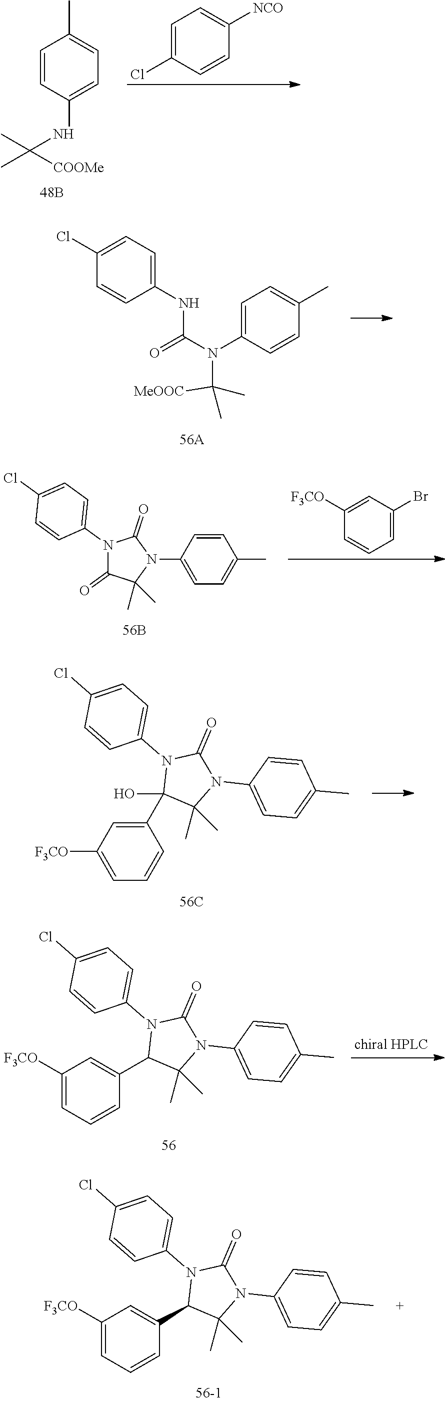

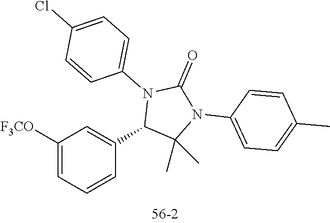 [SMILES: Cc1ccc(N2C(=O)N(c3ccc(Cl)cc3)[C@@H](c3cccc(OC(F)(F)F)c3)C2(C)C)cc1]

Compounds 56A, 56B, 56C, and 56 were synthesized by employing the procedures described for Compounds 12D, 12E, 6D, and 6 using Compounds 48B, 56A, 56B, 1-bromo-3-(trifluoromethoxy)benzene, and 56C at room temperature in lieu of Compounds 12B, 12C, 6C, 1-bromo-3-(trifluoromethyl)benzene, and 6D at 10° C.

Compound 56A. LC-MS (ESI) m/z: 361 [M+H]⁺.

Compound 56B. LC-MS (ESI) m/z: 329 [M+H]⁺.

Compound 56C. LC-MS (ESI) m/z: 491 [M+H]⁺. ¹H-NMR (CDCl₃, 400 MHz): δ (ppm) 0.83 (s, 3H), 1.32 (s, 3H), 2.38 (s, 3H), 3.82 (s, 1H), 7.12-7.18 (m, 5H), 7.21 (d, J=6.0 Hz, 2H), 7.32-7.33 (m, 3H), 7.41 (d, J=7.2 Hz, 2H).

Compound 56. LC-MS (ESI) m/z: 475 [M+H]⁺. Compound 56 was separated with chiral HPLC to yield Compound 56-1 and Compound 56-2.

Compound 56-1: LC-MS (ESI) m/z: 475 [M+H]⁺; ¹H-NMR (CDCl₃, 400 MHz): δ (ppm) 0.78 (s, 3H), 1.51 (s, 3H), 2.37 (s, 3H), 4.96 (s, 1H), 7.11 (d, J=6.4 Hz, 2H), 7.15 (s, 1H), 7.17-7.19 (m, 3H), 7.21-7.23 (m, 3H), 7.37-7.39 (m, 3H). Chiral separation conditions: MeOH contained 0.2% Methanol Ammonia; OZ-H (100×4.6 mm, 5 μm); retention time: 0.73 minutes.

Compound 56-2: LC-MS (ESI) m/z: 475 [M+H]⁺; ¹H-NMR (CDCl₃, 400 MHz): δ (ppm) 0.78 (s, 3H), 1.51 (s, 3H), 2.37 (s, 3H), 4.96 (s, 1H), 7.11 (d, J=6.8 Hz, 2H), 7.15 (s, 1H), 7.17-7.19 (m, 3H), 7.21-7.23 (m, 3H), 7.37-7.39 (m, 3H). Chiral separation condition MeOH contained 0.2% Methanol Ammonia; OZ-H (100×4.6 mm, 5 μm); retention time: 1.22 minutes.

### Example 57

### Synthesis of 3-(5-(4-cyanophenyl)-6-oxo-7-(p-tolyl)-2-oxa-5,7-diazaspiro[3.4]octan-8-yl)benzonitrile (57), (R)-3-(5-(4-cyanophenyl)-6-oxo-7-(p-tolyl)-2-oxa-5,7-diazaspiro[3.4]octan-8-yl)benzonitrile (57-1), and (S)-3-(5-(4-cyanophenyl)-6-oxo-7-(p-tolyl)-2-oxa-5,7-diazaspiro[3.4]octan-8-yl)benzonitrile (57-2)

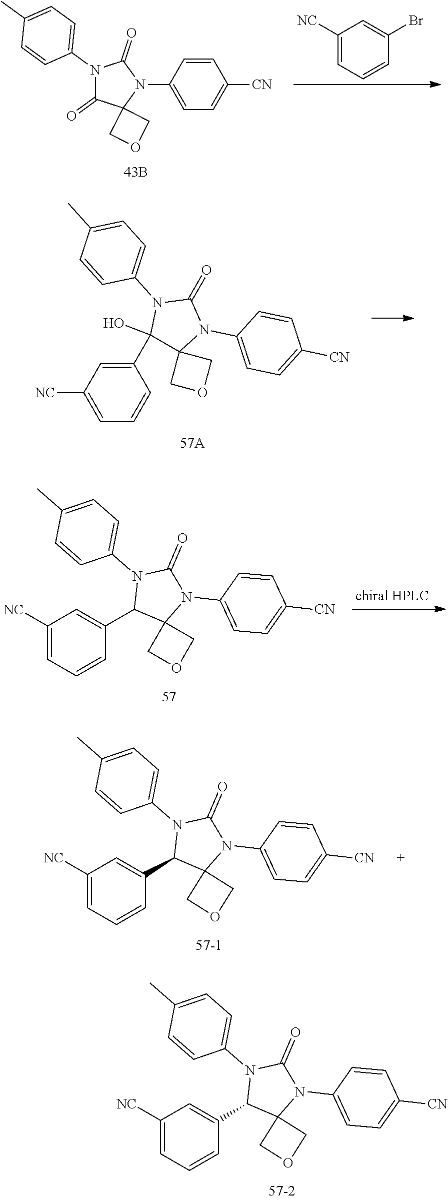 [SMILES: Cc1ccc(N2C(=O)N(c3ccc(C#N)cc3)C3(COC3)C2=O)cc1 ; N#Cc1cccc(Br)c1 ; Cc1ccc(N2C(=O)N(c3ccc(C#N)cc3)C3(COC3)C2(O)c2cccc(C#N)c2)cc1 ; Cc1ccc(N2C(=O)N(c3ccc(C#N)cc3)C3(COC3)C2c2cccc(C#N)c2)cc1 ; Cc1ccc(N2C(=O)N(c3ccc(C#N)cc3)C3(COC3)[C@H]2c2cccc(C#N)c2)cc1 ; Cc1ccc(N2C(=O)N(c3ccc(C#N)cc3)C3(COC3)[C@@H]2c2cccc(C#N)c2)cc1]

Compounds 57A and 57 were synthesized by employing the procedures described for Compounds 6D and 29 using Compounds 43B, 3-bromobenzonitrile, and 57A in lieu of Compounds 6C, 1-bromo-3-(trifluoromethyl)benzene, and 29F.

Compound 57A. LC-MS (ESI) m/z: 437 [M+H]⁺; ¹H-NMR (CDCl₃, 400 MHz): δ (ppm) 2.28 (s, 3H), 4.09 (d, J=8.0 Hz, 1H), 4.57-4.60 (m, 2H), 4.75 (d, J=8.8 Hz, 1H), 5.21 (d, J=8.8 Hz, 1H), 7.04 (d, J=8.4 Hz, 2H), 7.17 (d, J=8.4 Hz, 2H), 7.49 (t, J=7.6 Hz, 1H), 7.65-7.74 (m, 6H), 7.89 (s, 1H).

Compound 57 was separated with chiral HPLC to yield Compound 57-1 and Compound 57-2.

Compound 57-1: LC-MS (ESI) m/z: 421 [M+H]⁺; ¹H-NMR (CDCl₃, 400 MHz): δ (ppm) 2.27 (s, 3H), 4.22 (d, J=8.4 Hz, 1H), 4.62 (d, J=8.4 Hz, 1H), 4.93 (d, J=7.6 Hz, 1H), 5.14 (d, J=7.6 Hz, 1H), 5.44 (s, 1H), 7.08 (d, J=8.4 Hz, 2H), 7.24 (d, J=8.8 Hz, 2H), 7.53-7.57 (m, 1H), 7.60-7.62 (m, 1H), 7.66-7.68 (m, 2H), 7.73-7.80 (m, 4H). Chiral separation conditions: MeOH contained 0.2% Methanol Ammonia; OZ-H (4.6×100 mm, 5 μm); retention time: 2.33 minutes.

Compound 57-2: LC-MS (ESI) m/z: 421 [M+H]⁺; ¹H-NMR (CDCl₃, 400 MHz): δ (ppm) 2.27 (s, 3H), 4.22 (d, J=8.8 Hz, 1H), 4.62 (d, J=8.4 Hz, 1H), 4.93 (d, J=7.6 Hz, 1H), 5.14 (d, J=8.0 Hz, 1H), 5.44 (s, 1H), 7.08 (d, J=8.4 Hz, 2H), 7.24 (d, J=8.8 Hz, 2H), 7.53-7.57 (m, 1H), 7.60-7.62 (m, 1H), 7.66-7.69 (m, 2H), 7.73-7.80 (m, 4H); Chiral separation conditions: MeOH contained 0.2% Methanol Ammonia; OZ-H (4.6×100 mm, 5 m); retention time: 3.27 minutes.

### BIOLOGICAL EXAMPLES

The following examples describe ways in which the compounds described herein were tested to measure in vitro activity in enzymatic assays. A person of ordinary skill in the art would know that variations in the assay conditions could be used to determine the activity of the compounds.

### Biological Example 1

### Assay 1: CGT Enzymatic Assay

Full-length human CGT cDNA (see Appendix for sequence) was cloned into the BamH1/Xho1 site of pcDNA3.1 (+) mammalian expression vector (V90-20, Invitrogen, Carlsbad, Calif.) and plasmid was transfected into Chinese hamster ovary (CHO) cells. Lysate was prepared using M-PER (Mammalian Protein Extraction Reagent, ThermoFisher Scientific, Grand Island, N.Y.) in the presence of a protease inhibitor cocktail (P8340, Sigma, Saint Louis, Mo.). Each 100 mm dish (100% confluent, approximately 1×10⁷ cells) was lysed with 250 μl of M-PER containing protease inhibitors. Protein concentration was determined using Pierce BCA protein assay kit (ThermoFisher Scientific). Four micrograms of CHO/CGT lysate was incubated with various concentrations of a compound (0.001 μM-50 μM in DMSO) in 10 mM HEPES (pH 7.2) containing 35 μM dioleoylphosphatidylcholine, 5 mM MgCl₂, 5 mM MnCl₂, 1% BSA, 15 mM KCl, 1 mM EGTA, 8 mM CHAPS, 10 μM C6-NBD-dihydro-ceremide, 17.5 μM UDP-galactose, and 0.01% Tween 80 in a final reaction volume of 20 μl at 37° C. for 1 hour. The final concentration of DMSO was 0.5% in both compound treated and mock treated samples. Each individual reaction was diluted with 80 μl of methanol:acetronitrile (1:3) containing 5 μM N-docdecanoyl-NBD-galactosylceramide (internal standard) to stop the reaction and then 200 μl H₂O:acetonitile (1:1) was added to precipitate the protein. After sufficient mixing, plates were centrifuged at 2469 g for 38 mins, 200 μl of supernatant was transferred to a LC/MS 96-deep well plate and a second spin was performed at 2469 g for 10 mins. Final supernatant was injected in triplicate for Rapidfire/MS/MS analysis.

The quantitative analysis of C6-NBD-dihydro-ceremide and C6-NBD-dihydro-galactosylceramide was performed on a Rapid Fire 360 high-throughput mass spectrometry system (Agilent Technologies, Palo Alto, Calif.) coupled with a API4000⁺ triple quadrupole mass spectrometer (Applied Biosystems, Concord, Ontario, Canada). RapidFire software packages including RapidFire Control panel, RapidFire UI and RapidFire Integrator (Agilent Technologies) were used to control RapidFire and process data. Analyst 1.6.2 software packages (Applied Biosystems) were used to control MS system and acquire MS data. Ten μL of sample was loaded on a micro-scale C4 solid-phase extraction (SPE) cartridge (Agilent Technologies) and salts were removed using water supplemented with 0.1% formic acid at the flow rate of 1.5 mL/min in 3 sec. C6-NBD-dihydro-ceremide and C6-NBD-dihydro-galactosylceramide were co-eluted into the mass spectrometer using acetonitrile containing 0.1% formic acid at the flow rate of 1.0 mL/min for 3 sec. The total cycle time of one injection was 8 secs. The MS/MS detection was performed in ESI negative mode. The mass transition of C6-NBD-dihydro-galactosylceramide was m/z 752.6→678.6 using a −40 V collision energy, the mass transition of C6-NBD-dihydro-ceramide was m/z 590.6→115.8 using a −40 V collision energy and the mass transition of N-docdecanoyl-NBD-galactosylceramide was m/z 820.9→746.3 using a −45 V collision energy.

The C6-NBD-dihydro-galactosylceramide reading was normalized first by dividing the peak area of C6-NBD-dihydro-galactosylceramide by the peak area of the internal standard, N-docdecanoyl-NBD-galactosylceramide. IC₅₀ values were generated from sigmoidal dose-response (variable slope) curves with GraphPad Prism software (GraphPad Software, Inc., San Diego, Calif.) using the normalized peak areas of C6-NBD-dihydro-galactosylceramide or the percent inhibition of C6-NBD-dihydro-galactosylceramide accumulation relative to DMSO control.

Using the above assay, the following compounds were tested. In Table 1, biological data for the IC₅₀ values is provided as follows:

A is <1 M;

B is >1 to 10 M;

C is >10 to 50 M;

D is >50 to 100 M;

NA is not active under the current assay conditions; and

NT is not tested.

TABLE 1
Compounds of Formula (I) and Formula (II)

| Example | | |
|---|---|---|
| No. | Name | IC₅₀ |
| 1 | 1,3-bis(4-bromophenyl)-4-hydroxy-4-phenylimidazolidin-2-one | B |
| 2 | 1,3-bis(4-bromophenyl)-4-hydroxy-4-methylimidazolidin-2-one | NA |
| 3 | 3-(1,3-bis(4-chlorophenyl)-5-methyl-2-oxo-2,3-dihydro-1H- | A |
| | imidazol-4-yl)benzonitrile | |
| 4 | 1,3-bis(4-bromophenyl)-4-ethyl-4-hydroxyimidazolidin-2-one | C |
| 5 | 1,3-bis(4-bromophenyl)-4-(5-chlorothiophen-2-yl)-4- | C |
| | hydroxyimidazolidin-2-one | |
| 6 | 1,3-bis(4-bromophenyl)-4-butyl-4-hydroxyimidazolidin-2-one | NA |
| 7 | 1,3-bis(4-bromophenyl)-4-hydroxy-4-(thiophen-2-yl)imidazolidin- | B |
| | 2-one | |
| 8 | 4-(benzo[b]thiophen-2-yl)-1,3-bis(4-bromophenyl)-4- | D |
| | hydroxyimidazolidin-2-one | |
| 9 | 1,3-bis(4-bromophenyl)-4-hydroxy-4-(pyridin-2-yl)imidazolidin-2- | NA |
| | one | |
| 10 | 4-(benzo[d]thiazol-2-yl)-1,3-bis(4-bromophenyl)-4- | NA |
| | hydroxyimidazolidin-2-one | |
| 11 | 1,3-bis(4-fluorophenyl)-4-hydroxy-4-phenylimidazolidin-2-one | NA |
| 12 | 1,3-bis(4-chlorophenyl)-4-hydroxy-4-phenylimidazolidin-2-one | B |
| 13 | 1,3-bis(4-bromophenyl)-4-hydroxy-5-methyl-4- | A |
| | phenylimidazolidin-2-one | |
| 13-1 | (5S)-1,3-bis(4-bromophenyl)-4-hydroxy-5-methyl-4- | A |
| | phenylimidazolidin-2-one | |
| 13-2 | (5R)-1,3-bis(4-bromophenyl)-4-hydroxy-5-methyl-4- | A |
| | phenylimidazolidin-2-one | |
| 14 | 1,3-bis(4-bromophenyl)-4-hydroxy-4-(o-tolyl)imidazolidin-2-one | NA |
| 15 | 4-hydroxy-4-phenyl-1,3-bis(4- | NA |
| | (trifluoromethyl)phenyl)imidazolidin-2-one | |
| 16 | 1,3-bis(4-bromophenyl)-4-hydroxy-4-(m-tolyl)imidazolidin-2-one | B |
| 17 | 4-hydroxy-4-phenyl-1,3-di-p-tolylimidazolidin-2-one | B |
| 18 | 1,3-bis(4-bromophenyl)-1,3,4,5,6,7-hexahydro-2H- | C |
| | benzo[d]imidazol-2-one | |
| 19 | 1,3-bis(4-bromophenyl)-4-(4-chlorophenyl)-4- | C |
| | hydroxyimidazolidin-2-one | |
| 20 | 3-(4-bromophenyl)-4-hydroxy-1,4-diphenylimidazolidin-2-one | NA |
| 21 | 4-hydroxy-1,3,4-triphenylimidazolidin-2-one | NA |
| 22 | 1,3-bis(4-bromophenyl)-4,5-dipropyl-1,3-dihydro-2H-imidazol-2- | NA |
| | one | |
| 23 | 1,3-bis(4-bromophenyl)-4-hydroxy-4-(p-tolyl)imidazolidin-2-one | NA |
| 24 | 1,3-bis(4-bromophenyl)-4-hydroxy-4-(naphthalen-2- | NA |
| | yl)imidazolidin-2-one | |
| 25 | 1,3-bis(4-bromophenyl)-5-ethyl-4-hydroxy-4-phenylimidazolidin- | A |
| | 2-one | |
| 25-2 | (5R)-1,3-bis(4-bromophenyl)-5-ethyl-4-hydroxy-4- | A |
| | phenylimidazolidin-2-one | |
| 25-1 | (5S)-1,3-bis(4-bromophenyl)-5-ethyl-4-hydroxy-4- | A |
| | phenylimidazolidin-2-one | |
| 26 | 1,3-bis(4-bromophenyl)-4-hydroxy-4-phenylimidazolidine-2-thione | D |
| 27 | 4-benzoyl-1,3-bis(4-chlorophenyl)-5-propyl-1,3-dihydro-2H- | C |
| | imidazol-2-one | |
| 28 | 1-(4-bromophenyl)-4-hydroxy-3,4-diphenylimidazolidin-2-one | C |
| 29-1 | (5S)-1,3-bis(4-bromophenyl)-4-hydroxy-5-methyl-4-(m- | A |
| | tolyl)imidazolidin-2-one | |
| 29-2 | (5R)-1,3-bis(4-bromophenyl)-4-hydroxy-5-methyl-4-(m- | A |
| | tolyl)imidazolidin-2-one | |
| 30 | 1,3-bis(4-bromophenyl)-4-hydroxy-4-(3- | B |
| | methoxyphenyl)imidazolidin-2-one | |
| 31 | 1,3-bis(4-bromophenyl)-4-hydroxy-4-(3- | A |
| | (trifluoromethyl)phenyl)imidazolidin-2-one | |
| 32 | 1,3-bis(3,4-dichlorophenyl)-4-hydroxy-4-phenylimidazolidin-2-one | B |
| 33 | 1,3-bis(4-bromophenyl)-4-(3,5-dimethylphenyl)-4- | C |
| | hydroxyimidazolidin-2-one | |
| 34 | 1,3-bis(4-chlorophenyl)-4-hydroxy-4-(m-tolyl)imidazolidin-2-one | B |
| 35 | 1,3-bis(4-bromophenyl)-4-(3-chlorophenyl)-4- | B |
| | hydroxyimidazolidin-2-one | |
| 36 | 1,3-bis(4-chlorophenyl)-4-hydroxy-5-methyl-4-(m-tolyl)imidazolidin-2-one | A |
| 36-1 | (5S)-1,3-bis(4-chlorophenyl)-4-hydroxy-5-methyl-4-(m- | A |
| | tolyl)imidazolidin-2-one | |
| 36-2 | (5R)-1,3-bis(4-chlorophenyl)-4-hydroxy-5-methyl-4-(m- | B |
| | tolyl)imidazolidin-2-one | |
| 37 | 1,3-bis(4-chlorophenyl)-4-(3-(1-hydroxyethyl)phenyl)-5-methyl- | B |
| | 1,3-dihydro-2H-imidazol-2-one | |
| 38 | 3-(4-bromophenyl)-1-(4-chlorophenyl)-4-hydroxy-4- | C |
| | phenylimidazolidin-2-one | |
| 39 | 4-(3-chlorophenyl)-1,3-bis(4-chlorophenyl)-4- | A |
| | hydroxyimidazolidin-2-one | |
| 40 | 1,3-bis(4-chlorophenyl)-4-ethyl-5-(3-(trifluoromethyl)phenyl)-1,3- | A |
| | dihydro-2H-imidazol-2-one | |
| 41 | 1,3-bis(4-chlorophenyl)-4-(methoxymethyl)-5-(3- | A |
| | (trifluoromethyl)phenyl)-1,3-dihydro-2H-imidazol-2-one | |
| 42 | 1,3-bis(4-bromophenyl)-4-(3,5-dimethylphenyl)-4-hydroxy-5- | A |
| | methylimidazolidin-2-one | |
| 42-2 | (5R)-1,3-bis(4-bromophenyl)-4-(3,5-dimethylphenyl)-4-hydroxy-5- | B |
| | methylimidazolidin-2-one | |
| 42-1 | (5S)-1,3-bis(4-bromophenyl)-4-(3,5-dimethylphenyl)-4-hydroxy-5- | A |
| | methylimidazolidin-2-one | |
| 43 | 1-(4-bromophenyl)-3-(4-chlorophenyl)-4-hydroxy-4- | B |
| | phenylimidazolidin-2-one | |
| 44 | 3-(1,3-bis(4-chlorophenyl)-5-isopropyl-2-oxo-2,3-dihydro-1H- | A |
| | imidazol-4-yl)benzonitrile | |
| 45 | 1,3-bis(4-bromophenyl)-4-ethyl-4-hydroxy-5-phenylimidazolidin- | B |
| | 2-one | |
| 45-2 | (5R)-1,3-bis(4-bromophenyl)-4-ethyl-4-hydroxy-5- | C |
| | phenylimidazolidin-2-one | |
| 45-1 | (5S)-1,3-bis(4-bromophenyl)-4-ethyl-4-hydroxy-5- | A |
| | phenylimidazolidin-2-one | |
| 46 | 3-(1,3-bis(4-bromophenyl)-5-isopropyl-2-oxo-2,3-dihydro-1H- | A |
| | imidazol-4-yl)benzonitrile | |
| 47 | 1,3-bis(4-bromophenyl)-4-(3-ethylphenyl)-4-hydroxyimidazolidin- | B |
| | 2-one | |
| 48 | 3-(1,3-bis(4-chlorophenyl)-5-cyclopropyl-2-oxo-2,3-dihydro-1H- | A |
| | imidazol-4-yl)benzonitrile | |
| 49 | 1,3-bis(4-bromophenyl)-4-hydroxy-5-methyl-4-(thiophen-3- | B |
| | yl)imidazolidin-2-one | |
| 49-2 | (5R)-1,3-bis(4-bromophenyl)-4-hydroxy-5-methyl-4-(thiophen-2- | B |
| | yl)imidazolidin-2-one | |
| 49-1 | (5S)-1,3-bis(4-bromophenyl)-4-hydroxy-5-methyl-4-(thiophen-2- | B |
| | yl)imidazolidin-2-one | |
| 50 | 1,3-bis(4-chlorophenyl)-4-isopropyl-5-(3- | A |
| | (trifluoromethoxy)phenyl)-1,3-dihydro-2H-imidazol-2-one | |
| 51 | 1,3-bis(4-bromophenyl)-4-hydroxy-4-phenyl-5-propylimidazolidin- | A |
| | 2-one | |
| 51-2 | (5R)-1,3-bis(4-bromophenyl)-4-hydroxy-4-phenyl-5- | A |
| | propylimidazolidin-2-one | |
| 51-1 | (5S)-1,3-bis(4-bromophenyl)-4-hydroxy-4-phenyl-5- | B |
| | propylimidazolidin-2-one | |
| 52 | 1,3-bis(4-bromophenyl)-4-hydroxy-4-(pyridin-3-yl)imidazolidin-2- | C |
| | one | |
| 53-1 | (5S)-1,3-bis(4-chlorophenyl)-4-hydroxy-5-methyl-4- | A |
| | phenylimidazolidin-2-one | |
| 53-2 | (5R)-1,3-bis(4-chlorophenyl)-4-hydroxy-5-methyl-4- | A |
| | phenylimidazolidin-2-one | |
| 54 | 1,3-bis(4-bromophenyl)-4-hydroxy-4-(6-methoxypyridin-3- | NA |
| | yl)imidazolidin-2-one | |
| 55 | 4-(3-bromophenyl)-1,3-bis(4-chlorophenyl)-4- | B |
| | hydroxyimidazolidin-2-one | |
| 56 | 1,3-bis(4-chlorophenyl)-4-ethyl-4-hydroxyimidazolidin-2-one | NA |
| 57 | 1,3-bis(2,4-dichlorophenyl)-4-hydroxy-4-phenylimidazolidin-2-one | NA |
| 58 | 1,3-bis(4-bromophenyl)-4-hydroxy-4-(3- | A |
| | (trifluoromethoxy)phenyl)imidazolidin-2-one | |
| 59 | 1,3-bis(4-bromophenyl)-4-hydroxy-4-(3- | B |
| | isopropylphenyl)imidazolidin-2-one | |
| 60 | 1,3-bis(4-chlorophenyl)-4-(3-ethynylphenyl)-4- | A |
| | hydroxyimidazolidin-2-one | |
| 61 | 4-(1,3-bis(4-bromophenyl)-4-hydroxy-2-oxoimidazolidin-4- | NA |
| | yl)benzonitrile | |
| 62 | 4-(3-chlorophenyl)-1,3-bis(4-chlorophenyl)-4-hydroxy-5- | A |
| | methylimidazolidin-2-one | |
| 62-1 | (5S)-4-(3-chlorophenyl)-1,3-bis(4-chlorophenyl)-4-hydroxy-5- | A |
| | methylimidazolidin-2-one | |
| 62-2 | (5R)-4-(3-chlorophenyl)-1,3-bis(4-chlorophenyl)-4-hydroxy-5- | A |
| | methylimidazolidin-2-one | |
| 63 | 3-(1,3-bis(4-chlorophenyl)-4-hydroxy-2-oxoimidazolidin-4- | A |
| | yl)benzonitrile | |
| 64 | 1,3-bis(4-chlorophenyl)-4-(3-fluorophenyl)-4- | B |
| | hydroxyimidazolidin-2-one | |
| 65 | 1,3-bis(4-chlorophenyl)-4-cyclopropyl-5-(3- | A |
| | (trifluoromethoxy)phenyl)-1,3-dihydro-2H-imidazol-2-one | |
| 66 | 1,3-bis(4-bromophenyl)-4-hydroxy-5-methyl-4-(3- | A |
| | (trifluoromethoxy)phenyl)imidazolidin-2-one | |
| 66-1 | (5S)-1,3-bis(4-bromophenyl)-4-hydroxy-5-methyl-4-(3- | A |
| | (trifluoromethoxy)phenyl)imidazolidin-2-one | |
| 66-2 | (5R)-1,3-bis(4-bromophenyl)-4-hydroxy-5-methyl-4-(3- | A |
| | (trifluoromethoxy)phenyl)imidazolidin-2-one | |
| 67 | 1,3-bis(4-bromophenyl)-5-ethyl-4-hydroxy-4-(3- | A |
| | (trifluoromethyl)phenyl)imidazolidin-2-one | |
| 67-1 | (5S)-1,3-bis(4-bromophenyl)-5-ethyl-4-hydroxy-4-(3- | A |
| | (trifluoromethyl)phenyl)imidazolidin-2-one | |
| 67-2 | (5R)-1,3-bis(4-bromophenyl)-5-ethyl-4-hydroxy-4-(3- | A |
| | (trifluoromethyl)phenyl)imidazolidin-2-one | |
| 68 | 1,3-bis(4-bromophenyl)-4-(3-ethylphenyl)-4-hydroxy-5- | A |
| | methylimidazolidin-2-one | |
| 68-1 | (5S)-1,3-bis(4-bromophenyl)-4-(3-ethylphenyl)-4-hydroxy-5- | A |
| | methylimidazolidin-2-one | |
| 68-2 | (5R)-1,3-bis(4-bromophenyl)-4-(3-ethylphenyl)-4-hydroxy-5- | A |
| | methylimidazolidin-2-one | |
| 69 | 1,3-bis(4-chlorophenyl)-4-(3-ethylphenyl)-4-hydroxy-5- | A |
| | methylimidazolidin-2-one | |
| 69-1 | (5S)-1,3-bis(4-chlorophenyl)-4-(3-ethylphenyl)-4-hydroxy-5- | A |
| | methylimidazolidin-2-one | |
| 69-2 | (5R)-1,3-bis(4-chlorophenyl)-4-(3-ethylphenyl)-4-hydroxy-5- | A |
| | methylimidazolidin-2-one | |
| 70 | 1,3-bis(4-chlorophenyl)-4-cyclopropyl-5-(3- | A |
| | (difluoromethoxy)phenyl)-1,3-dihydro-2H-imidazol-2-one | |
| 71-1 | (5S)-1,3-bis(4-bromophenyl)-4-hydroxy-4-(3-methoxyphenyl)-5- | A |
| | methylimidazolidin-2-one | |
| 71-2 | (5R)-1,3-bis(4-bromophenyl)-4-hydroxy-4-(3-methoxyphenyl)-5- | A |
| | methylimidazolidin-2-one | |
| 72 | 3-(5,7-bis(4-chlorophenyl)-8-hydroxy-6-oxo-5,7- | A |
| | diazaspiro[3.4]octan-8-yl)benzonitrile | |
| 73 | 1,3-bis(4-bromophenyl)-4-(3-chlorophenyl)-4-hydroxy-5- | A |
| | methylimidazolidin-2-one | |
| 73-1 | (5S)-1,3-bis(4-bromophenyl)-4-(3-chlorophenyl)-4-hydroxy-5- | A |
| | methylimidazolidin-2-one | |
| 73-2 | (5R)-1,3-bis(4-bromophenyl)-4-(3-chlorophenyl)-4-hydroxy-5- | A |
| | methylimidazolidin-2-one | |
| 74-1 | (5S)-1,3-bis(4-bromophenyl)-4-hydroxy-5-methyl-4-(3- | A |
| | (trifluoromethyl)phenyl)imidazolidin-2-one | |
| 74-2 | (5R)-1,3-bis(4-bromophenyl)-4-hydroxy-5-methyl-4-(3- | A |
| | (trifluoromethyl)phenyl)imidazolidin-2-one | |
| 75-1 | (5S)-1,3-bis(4-chlorophenyl)-5-ethyl-4-hydroxy-4-(m- | A |
| | tolyl)imidazolidin-2-one | |
| 75-2 | (5R)-1,3-bis(4-chlorophenyl)-5-ethyl-4-hydroxy-4-(m- | A |
| | tolyl)imidazolidin-2-one | |
| 76-1 | (5S)-1,3-bis(4-bromophenyl)-5-ethyl-4-hydroxy-4-(m- | A |
| | tolyl)imidazolidin-2-one | |
| 76-2 | (5R)-1,3-bis(4-bromophenyl)-5-ethyl-4-hydroxy-4-(m- | A |
| | tolyl)imidazolidin-2-one | |
| 77-1 | (5S)-1,3-bis(4-chlorophenyl)-4-hydroxy-5-methyl-4-(3- | A |
| | (trifluoromethyl)phenyl)imidazolidin-2-one | |
| 77-2 | (5R)-1,3-bis(4-chlorophenyl)-4-hydroxy-5-methyl-4-(3- | A |
| | (trifluoromethyl)phenyl)imidazolidin-2-one | |
| 78 | 1,3-bis(4-chlorophenyl)-4-(3-cyclopropylphenyl)-4- | B |
| | hydroxyimidazolidin-2-one | |
| 79 | 1,3-bis(4-bromophenyl)-4-(3-ethoxyphenyl)-4- | B |
| | hydroxyimidazolidin-2-one | |
| 80-1 | (5S)-1,3-bis(4-chlorophenyl)-5-ethyl-4-hydroxy-4-(3- | A |
| | (trifluoromethyl)phenyl)imidazolidin-2-one | |
| 80-2 | (5R)-1,3-bis(4-chlorophenyl)-5-ethyl-4-hydroxy-4-(3- | A |
| | (trifluoromethyl)phenyl)imidazolidin-2-one | |
| 81 | 4-(3-chlorophenyl)-1,3-bis(4-chlorophenyl)-5-ethyl-4- | A |
| | hydroxyimidazolidin-2-one | |
| 81-1 | (5S)-4-(3-chlorophenyl)-1,3-bis(4-chlorophenyl)-5-ethyl-4- | A |
| | hydroxyimidazolidin-2-one | |
| 81-2 | (5R)-4-(3-chlorophenyl)-1,3-bis(4-chlorophenyl)-5-ethyl-4- | A |
| | hydroxyimidazolidin-2-one | |
| 82 | 1,3-bis(4-chlorophenyl)-5-ethyl-4-hydroxy-4-(3- | A |
| | (trifluoromethoxy)phenyl)imidazolidin-2-one | |
| 82-1 | (5R)-1,3-bis(4-chlorophenyl)-5-ethyl-4-hydroxy-4-(3- | A |
| | (trifluoromethoxy)phenyl)imidazolidin-2-one | |
| 82-2 | (5S)-1,3-bis(4-chlorophenyl)-5-ethyl-4-hydroxy-4-(3- | A |
| | (trifluoromethoxy)phenyl)imidazolidin-2-one | |
| 83 | 1,3-bis(4-bromophenyl)-5-ethyl-4-hydroxy-4-(3- | A |
| | (trifluoromethoxy)phenyl)imidazolidin-2-one | |
| 83-1 | (5R)-1,3-bis(4-bromophenyl)-5-ethyl-4-hydroxy-4-(3- | A |
| | (trifluoromethoxy)phenyl)imidazolidin-2-one | |
| 83-2 | (5S)-1,3-bis(4-bromophenyl)-5-ethyl-4-hydroxy-4-(3- | A |
| | (trifluoromethoxy)phenyl)imidazolidin-2-one | |
| 84-1 | methyl(5S)-1,3-bis(4-bromophenyl)-4-hydroxy-5-methyl-2- | NA |
| | oxoimidazolidine-4-carboxylate | |
| 84-2 | methyl(5R)-1,3-bis(4-bromophenyl)-4-hydroxy-5-methyl-2- | NA |
| | oxoimidazolidine-4-carboxylate | |
| 85 | 1,3-bis(4-chlorophenyl)-4-(3,5-dichlorophenyl)-4- | B |
| | hydroxyimidazolidin-2-one | |
| 86-1 | (5S)-1,3-bis(4-bromophenyl)-4-hydroxy-4,5-diphenylimidazolidin- | C |
| | 2-one | |
| 86-2 | (5R)-1,3-bis(4-bromophenyl)-4-hydroxy-4,5-diphenylimidazolidin- | B |
| | 2-one | |
| 87 | 1,3-bis(4-chlorophenyl)-4-hydroxy-5-propyl-4-(3- | A |
| | (trifluoromethyl)phenyl)imidazolidin-2-one | |
| 87-1 | (5S)-1,3-bis(4-chlorophenyl)-4-hydroxy-5-propyl-4-(3- | A |
| | (trifluoromethyl)phenyl)imidazolidin-2-one | |
| 87-2 | (5R)-1,3-bis(4-chlorophenyl)-4-hydroxy-5-propyl-4-(3- | A |
| | (trifluoromethyl)phenyl)imidazolidin-2-one | |
| 88 | 1,3-bis(4-bromophenyl)-4-(3-ethylphenyl)-4-hydroxy-5- | A |
| | propylimidazolidin-2-one | |
| 88-1 | (5S)-1,3-bis(4-bromophenyl)-4-(3-ethylphenyl)-4-hydroxy-5- | A |
| | propylimidazolidin-2-one | |
| 88-2 | (5R)-1,3-bis(4-bromophenyl)-4-(3-ethylphenyl)-4-hydroxy-5- | A |
| | propylimidazolidin-2-one | |
| 89 | 1,3-bis(4-chlorophenyl)-4-(3-ethylphenyl)-4-hydroxy-5- | A |
| | propylimidazolidin-2-one | |
| 89-1 | (5S)-1,3-bis(4-chlorophenyl)-4-(3-ethylphenyl)-4-hydroxy-5- | A |
| | propylimidazolidin-2-one | |
| 89-2 | (5R)-1,3-bis(4-chlorophenyl)-4-(3-ethylphenyl)-4-hydroxy-5- | A |
| | propylimidazolidin-2-one | |
| 90 | 1,3-bis(4-chlorophenyl)-4-hydroxy-5-propyl-4-(m- | A |
| | tolyl)imidazolidin-2-one | |
| 90-1 | (5S)-1,3-bis(4-chlorophenyl)-4-hydroxy-5-propyl-4-(m- | A |
| | tolyl)imidazolidin-2-one | |
| 90-2 | (5R)-1,3-bis(4-chlorophenyl)-4-hydroxy-5-propyl-4-(m- | A |
| | tolyl)imidazolidin-2-one | |
| 91-1 | (5S)-1,3-bis(4-chlorophenyl)-4-hydroxy-5-propyl-4-(3- | A |
| | (trifluoromethoxy)phenyl)imidazolidin-2-one | |
| 91-2 | (5R)-1,3-bis(4-chlorophenyl)-4-hydroxy-5-propyl-4-(3- | A |
| | (trifluoromethoxy)phenyl)imidazolidin-2-one | |
| 92-1 | (5S)-1,3-bis(4-chlorophenyl)-4-hydroxy-5-(methoxymethyl)-4-(3- | A |
| | (trifluoromethyl)phenyl)imidazolidin-2-one | |
| 92-2 | (5R)-1,3-bis(4-chlorophenyl)-4-hydroxy-5-(methoxymethyl)-4-(3- | A |
| | (trifluoromethyl)phenyl)imidazolidin-2-one | |
| 93 | 1,3-bis(4-chlorophenyl)-4-(3-ethynylphenyl)-4-hydroxy-5- | A |
| | methylimidazolidin-2-one | |
| 93-1 | (5S)-1,3-bis(4-chlorophenyl)-4-(3-ethynylphenyl)-4-hydroxy-5- | A |
| | methylimidazolidin-2-one | |
| 93-2 | (5R)-1,3-bis(4-chlorophenyl)-4-(3-ethynylphenyl)-4-hydroxy-5- | A |
| | methylimidazolidin-2-one | |
| 94 | 4-(3-bromophenyl)-1,3-bis(4-chlorophenyl)-4-hydroxy-5- | A |
| | methylimidazolidin-2-one | |
| 94-1 | (5S)-4-(3-bromophenyl)-1,3-bis(4-chlorophenyl)-4-hydroxy-5- | A |
| | methylimidazolidin-2-one | |
| 94-2 | (5R)-4-(3-bromophenyl)-1,3-bis(4-chlorophenyl)-4-hydroxy-5- | A |
| | methylimidazolidin-2-one | |
| 95 | 3-(1,3-bis(4-chlorophenyl)-4-hydroxy-2-oxoimidazolidin-4-yl)-5- | B |
| | chlorobenzonitrile | |
| 96 | 1,3-bis(4-bromophenyl)-4-hydroxy-5-propyl-4-(m- | A |
| | tolyl)imidazolidin-2-one | |
| 96-1 | (5S)-1,3-bis(4-bromophenyl)-4-hydroxy-5-propyl-4-(m- | A |
| | tolyl)imidazolidin-2-one | |
| 96-2 | (5R)-1,3-bis(4-bromophenyl)-4-hydroxy-5-propyl-4-(m- | A |
| | tolyl)imidazolidin-2-one | |
| 97 | 1,3-bis(4-bromophenyl)-4-hydroxy-5-methyl-4-(4-methylthiophen- | B |
| | 2-yl)imidazolidin-2-one | |
| 97-1 | (5S)-1,3-bis(4-bromophenyl)-4-hydroxy-5-methyl-4-(4- | B |
| | methylthiophen-2-yl)imidazolidin-2-one | |
| 97-2 | (5R)-1,3-bis(4-bromophenyl)-4-hydroxy-5-methyl-4-(4- | B |
| | methylthiophen-2-yl)imidazolidin-2-one | |
| 98 | 4-(3-bromo-5-chlorophenyl)-1,3-bis(4-chlorophenyl)-4- | B |
| | hydroxyimidazolidin-2-one | |
| 99 | 3-(1,3-bis(4-chlorophenyl)-4-hydroxy-5-methyl-2-oxoimidazolidin- | A |
| | 4-yl)benzonitrile | |
| 99-1 | 3-((5S)-1,3-bis(4-chlorophenyl)-4-hydroxy-5-methyl-2- | A |
| | oxoimidazolidin-4-yl)benzonitrile | |
| 99-2 | 3-((5R)-1,3-bis(4-chlorophenyl)-4-hydroxy-5-methyl-2- | A |
| | oxoimidazolidin-4-yl)benzonitrile | |
| 100-1 | (5S)-1,3-bis(4-bromophenyl)-4-hydroxy-5-propyl-4-(3- | A |
| | (trifluoromethyl)phenyl)imidazolidin-2-one | |
| 100-2 | (5R)-1,3-bis(4-bromophenyl)-4-hydroxy-5-propyl-4-(3- | A |
| | (trifluoromethyl)phenyl)imidazolidin-2-one | |
| 101 | 3-(1,3-bis(4-chlorophenyl)-4-hydroxy-2-oxo-5-propylimidazolidin- | A |
| | 4-yl)benzonitrile | |
| 101-1 | 3-((5S)-1,3-bis(4-chlorophenyl)-4-hydroxy-2-oxo-5- | A |
| | propylimidazolidin-4-yl)benzonitrile | |
| 101-2 | 3-((5R)-1,3-bis(4-chlorophenyl)-4-hydroxy-2-oxo-5- | A |
| | propylimidazolidin-4-yl)benzonitrile | |
| 102 | 3-(1,3-bis(4-chlorophenyl)-4-hydroxy-2-oxoimidazolidin-4-yl)-5- | B |
| | methylbenzonitrile | |
| 103 | 1,3-bis(4-chlorophenyl)-4-hydroxy-5-methyl-4-(3-(prop-1-yn-1- | A |
| | yl)phenyl)imidazolidin-2-one | |
| 103-1 | (5S)-1,3-bis(4-chlorophenyl)-4-hydroxy-5-methyl-4-(3-(prop-1-yn- | A |
| | 1-yl)phenyl)imidazolidin-2-one | |
| 103-2 | (5R)-1,3-bis(4-chlorophenyl)-4-hydroxy-5-methyl-4-(3-(prop-1-yn- | A |
| | 1-yl)phenyl)imidazolidin-2-one | |
| 104 | 3-(1,3-bis(4-ethylphenyl)-4-hydroxy-5-methyl-2-oxoimidazolidin- | A |
| | 4-yl)benzonitrile | |
| 104-1 | 3-((5S)-1,3-bis(4-ethylphenyl)-4-hydroxy-5-methyl-2- | C |
| | oxoimidazolidin-4-yl)benzonitrile | |
| 104-2 | 3-((5R)-1,3-bis(4-ethylphenyl)-4-hydroxy-5-methyl-2- | A |
| | oxoimidazolidin-4-yl)benzonitrile | |
| 105 | 3-(4-hydroxy-5-methyl-2-oxo-1,3-di-p-tolylimidazolidin-4- | A |
| | yl)benzonitrile | |
| 105-1 | 3-((5S)-4-hydroxy-5-methyl-2-oxo-1,3-di-p-tolylimidazolidin-4- | B |
| | yl)benzonitrile | |
| 105-2 | 3-((5R)-4-hydroxy-5-methyl-2-oxo-1,3-di-p-tolylimidazolidin-4- | A |
| | yl)benzonitrile | |
| 106 | 3-(1,3-bis(4-chlorophenyl)-5-ethyl-4-hydroxy-2-oxoimidazolidin- | A |
| | 4-yl)benzonitrile | |
| 106-1 | 3-((5S)-1,3-bis(4-chlorophenyl)-5-ethyl-4-hydroxy-2- | A |
| | oxoimidazolidin-4-yl)benzonitrile | |
| 106-2 | 3-((5R)-1,3-bis(4-chlorophenyl)-5-ethyl-4-hydroxy-2- | A |
| | oxoimidazolidin-4-yl)benzonitrile | |
| 107 | 4-hydroxy-5-methyl-1,3-di-m-tolyl-4-(3- | A |
| | (trifluoromethoxy)phenyl)imidazolidin-2-one | |
| 108 | 4-(3-chloro-5-ethynylphenyl)-1,3-bis(4-chlorophenyl)-4- | B |
| | hydroxyimidazolidin-2-one | |
| 109 | 1,3-bis(4-chlorophenyl)-4-hydroxy-4-phenyl-5-(thiophen-2- | B |
| | yl)imidazolidin-2-one | |
| 110 | 3-(1-(4-bromophenyl)-3-(4-chlorophenyl)-4-hydroxy-2- | A |
| | oxoimidazolidin-4-yl)-5-chlorobenzonitrile | |
| 111 | 4,4′-(4-hydroxy-2-oxo-4-phenylimidazolidine-1,3- | NA |
| | diyl)dibenzonitrile | |
| 112-1 | (5S)-1,3-bis(4-bromophenyl)-4-hydroxy-5-propyl-4-(3- | A |
| | (trifluoromethoxy)phenyl)imidazolidin-2-one | |
| 112-2 | (5R)-1,3-bis(4-bromophenyl)-4-hydroxy-5-propyl-4-(3- | A |
| | (trifluoromethoxy)phenyl)imidazolidin-2-one | |
| 113 | 3-(1,3-bis(4-bromophenyl)-4-hydroxy-2-oxoimidazolidin-4-yl)-5- | B |
| | chlorobenzonitrile | |
| 114 | 4-(3-chlorophenyl)-1,3-bis(4-chlorophenyl)-4-hydroxy-5- | A |
| | propylimidazolidin-2-one | |
| 114-1 | (5S)-4-(3-chlorophenyl)-1,3-bis(4-chlorophenyl)-4-hydroxy-5- | A |
| | propylimidazolidin-2-one | |
| 114-2 | (5R)-4-(3-chlorophenyl)-1,3-bis(4-chlorophenyl)-4-hydroxy-5- | A |
| | propylimidazolidin-2-one | |
| 115 | 1,3-bis(4-bromophenyl)-5-cyclopropyl-4-hydroxy-4- | A |
| | phenylimidazolidin-2-one | |
| 115-1 | (5S)-1,3-bis(4-bromophenyl)-5-cyclopropyl-4-hydroxy-4- | A |
| | phenylimidazolidin-2-one | |
| 115-2 | (5R)-1,3-bis(4-bromophenyl)-5-cyclopropyl-4-hydroxy-4- | A |
| | phenylimidazolidin-2-one | |
| 116 | 3-(5-butyl-1,3-bis(4-chlorophenyl)-4-hydroxy-2-oxoimidazolidin- | A |
| | 4-yl)benzonitrile | |
| 116-1 | 3-((5S)-5-butyl-1,3-bis(4-chlorophenyl)-4-hydroxy-2- | A |
| | oxoimidazolidin-4-yl)benzonitrile | |
| 116-2 | 3-((5R)-5-butyl-1,3-bis(4-chlorophenyl)-4-hydroxy-2- | A |
| | oxoimidazolidin-4-yl)benzonitrile | |
| 117-1 | (5S)-4-(3-chlorophenyl)-1,3-bis(4-chlorophenyl)-4-hydroxy-5-((S)- | B |
| | tetrahydrofuran-2-yl)imidazolidin-2-one | |
| 117-2 | (5R)-4-(3-chlorophenyl)-1,3-bis(4-chlorophenyl)-4-hydroxy-5- | B |
| | ((R)-tetrahydrofuran-2-yl)imidazolidin-2-one | |
| 118 | 1,3-bis(4-chlorophenyl)-4-(3-(difluoromethoxy)phenyl)-5-ethyl-4- | A |
| | hydroxyimidazolidin-2-one | |
| 118-1 | (5S)-1,3-bis(4-chlorophenyl)-4-(3-(difluoromethoxy)phenyl)-5- | A |
| | ethyl-4-hydroxyimidazolidin-2-one | |
| 118-2 | (5R)-1,3-bis(4-chlorophenyl)-4-(3-(difluoromethoxy)phenyl)-5- | A |
| | ethyl-4-hydroxyimidazolidin-2-one | |
| 119 | 1,3-bis(4-bromophenyl)-4-(3-chlorophenyl)-4-hydroxy-5- | A |
| | propylimidazolidin-2-one | |
| 119-1 | (5S)-1,3-bis(4-bromophenyl)-4-(3-chlorophenyl)-4-hydroxy-5- | A |
| | propylimidazolidin-2-one | |
| 119-2 | (5R)-1,3-bis(4-bromophenyl)-4-(3-chlorophenyl)-4-hydroxy-5- | A |
| | propylimidazolidin-2-one | |
| 120 | 1,3-bis(4-bromophenyl)-4-hydroxy-5,5-dimethyl-4- | A |
| | phenylimidazolidin-2-one | |
| 121 | 1,3-bis(4-chlorophenyl)-4-hydroxy-5-methyl-4,5- | B |
| | diphenylimidazolidin-2-one | |
| 121-1 | (4S,5S)-1,3-bis(4-chlorophenyl)-4-hydroxy-5-methyl-4,5- | C |
| | diphenylimidazolidin-2-one | |
| 121-2 | (4R,5S)-1,3-bis(4-chlorophenyl)-4-hydroxy-5-methyl-4,5- | NA |
| | diphenylimidazolidin-2-one | |
| 121-3 | (4S,5R)-1,3-bis(4-chlorophenyl)-4-hydroxy-5-methyl-4,5- | NA |
| | diphenylimidazolidin-2-one | |
| 121-4 | (4R,5R)-1,3-bis(4-chlorophenyl)-4-hydroxy-5-methyl-4,5- | B |
| | diphenylimidazolidin-2-one | |
| 122 | 1,3-bis(4-chlorophenyl)-4-hydroxy-4,5-dipropylimidazolidin-2-one | C |
| 123-1 | 3-((5S)-5-ethyl-1,3-bis(4-fluorophenyl)-4-hydroxy-2- | B |
| | oxoimidazolidin-4-yl)benzonitrile | |
| 123-2 | 34(5R)-5-ethyl-1,3-bis(4-fluorophenyl)-4-hydroxy-2- | A |
| | oxoimidazolidin-4-yl)benzonitrile | |
| 124 | 4-(3-acetylphenyl)-1,3-bis(4-chlorophenyl)-4-hydroxy-5- | A |
| | methylimidazolidin-2-one | |
| 125 | 1,3-bis(4-bromophenyl)-4-(3-(dimethylamino)phenyl)-4- | NA |
| | hydroxyimidazolidin-2-one | |
| 126 | 1,3-bis(4-bromophenyl)-5-hydroxy-4-(3-methoxyphenyl)-4- | B |
| | methylimidazolidin-2-one | |
| 126-1 | (4S)-1,3-bis(4-bromophenyl)-5-hydroxy-4-(3-methoxyphenyl)-4- | NA |
| | methylimidazolidin-2-one | |
| 126-2 | (4R)-1,3-bis(4-bromophenyl)-5-hydroxy-4-(3-methoxyphenyl)-4- | A |
| | methylimidazolidin-2-one | |
| 127 | 1,3-bis(4-bromophenyl)-4-(3-(tert-butyl)phenyl)-4- | C |
| | hydroxyimidazolidin-2-one | |
| 128 | 4-(3-(tert-butyl)phenyl)-1,3-bis(4-chlorophenyl)-4- | C |
| | hydroxyimidazolidin-2-one | |
| 129 | 1,3-bis(4-chlorophenyl)-4-hydroxy-5-methyl-4-(3- | A |
| | (trifluoromethoxy)phenyl)imidazolidin-2-one | |
| 19-1 | (5S)-1,3-bis(4-chlorophenyl)-4-hydroxy-5-methyl-4-(3- | A |
| | (trifluoromethoxy)phenyl)imidazolidin-2-one | |
| 129-2 | (5R)-1,3-bis(4-chlorophenyl)-4-hydroxy-5-methyl-4-(3- | A |
| | (trifluoromethoxy)phenyl)imidazolidin-2-one | |
| 130 | 1,3-bis(4-fluorophenyl)-4-hydroxy-5-methyl-4-(3- | A |
| | (trifluoromethoxy)phenyl)imidazolidin-2-one | |
| 130-1 | (5S)-1,3-bis(4-fluorophenyl)-4-hydroxy-5-methyl-4-(3- | A |
| | (trifluoromethoxy)phenyl)imidazolidin-2-one | |
| 130-2 | (5R)-1,3-bis(4-fluorophenyl)-4-hydroxy-5-methyl-4-(3- | A |
| | (trifluoromethoxy)phenyl)imidazolidin-2-one | |
| 131 | 5-ethyl-1,3-bis(4-fluorophenyl)-4-hydroxy-4-(3- | A |
| | (trifluoromethoxy)phenyl)imidazolidin-2-one | |
| 132 | 3-(1,3-bis(4-chlorophenyl)-5-(cyclopropylmethyl)-4-hydroxy-2- | A |
| | oxoimidazolidin-4-yl)benzonitrile | |
| 132-1 | 3-((5S)-1,3-bis(4-chlorophenyl)-5-(cyclopropylmethyl)-4-hydroxy-B | |
| | 2-oxoimidazolidin-4-yl)benzonitrile | |
| 132-2 | 3-((5R)-1,3-bis(4-chlorophenyl)-5-(cyclopropylmethyl)-4-hydroxy- | A |
| | 2-oxoimidazolidin-4-yl)benzonitrile | |
| 133 | 1,3-bis(4-chlorophenyl)-4-hydroxy-5,5-dimethyl-4-(3- | A |
| | (trifluoromethoxy)phenyl)imidazolidin-2-one | |
| 134 | 1,3-bis(4-chlorophenyl)-4-hydroxy-5,5-dimethyl-4-(3- | A |
| | (trifluoromethyl)phenyl)imidazolidin-2-one | |
| 135 | 3-(1,3-bis(4-chlorophenyl)-4-hydroxy-2-oxo-1,3- | A |
| | diazaspiro[4.4]nonan-4-yl)benzonitrile | |
| 136-1 | (5S)-4-(3-acetylphenyl)-1,3-bis(4-chlorophenyl)-4-hydroxy-5- | B |
| | methylimidazolidin-2-one | |
| 136-2 | (5R)-4-(3-acetylphenyl)-1,3-bis(4-chlorophenyl)-4-hydroxy-5- | A |
| | methylimidazolidin-2-one | |
| 137 | 1,3-bis(4-chlorophenyl)-4-hydroxy-4-(3-(1-hydroxyethyl)phenyl)- | A |
| | 5-methylimidazolidin-2-one | |
| 138 | 3-(1,3-bis(4-chlorophenyl)-4-hydroxy-5,5-dimethyl-2- | A |
| | oxoimidazolidin-4-yl)benzonitrile | |
| 139 | 5-ethyl-1,3-bis(4-fluorophenyl)-4-hydroxy-4-(3- | B |
| | (trifluoromethyl)phenyl)imidazolidin-2-one | |
| 139-1 | (5S)-5-ethyl-1,3-bis(4-fluorophenyl)-4-hydroxy-4-(3- | B |
| | (trifluoromethyl)phenyl)imidazolidin-2-one | |
| 139-2 | (5R)-5-ethyl-1,3-bis(4-fluorophenyl)-4-hydroxy-4-(3- | A |
| | (trifluoromethyl)phenyl)imidazolidin-2-one | |
| 140 | 1,3-bis(4-chlorophenyl)-4-hydroxy-4-(3-(2-hydroxypropan-2- | B |
| | yl)phenyl)-5-methylimidazolidin-2-one | |
| 140-1 | (5S)-1,3-bis(4-chlorophenyl)-4-hydroxy-4-(3-(2-hydroxypropan-2- | NA |
| | yl)phenyl)-5-methylimidazolidin-2-one | |
| 140-2 | (5R)-1,3-bis(4-chlorophenyl)-4-hydroxy-4-(3-(2-hydroxypropan-2- | B |
| | yl)phenyl)-5-methylimidazolidin-2-one | |
| 141 | 1,3-bis(4-bromophenyl)-5-hydroxy-5-phenylimidazolidine-2,4- | C |
| | dione | |
| 143 | 1,3-bis(4-bromophenyl)-5-ethyl-5-hydroxyimidazolidine-2,4-dione | C |
| 144 | 1,3-bis(4-bromophenyl)-5-hydroxy-5-(4- | D |
| | methoxyphenyl)imidazolidine-2,4-dione | |
| 145 | 5-hydroxy-1,3-bis(4-isopropylphenyl)-5-phenylimidazolidine-2,4- | D |
| | dione | |
| 146 | 1,3-bis(4-chlorophenyl)-4-hydroxy-4-(3- | A |
| | (trifluoromethoxy)phenyl)-1,3-diazaspiro[4.4]nonan-2-one | |
| 147 | 1,3-bis(4-chlorophenyl)-5-hydroxy-5-phenylimidazolidine-2,4- | C |
| | dione | |
| 148 | 1,3-bis(4-bromophenyl)-5-hydroxy-5-(naphthalen-2- | D |
| | yl)imidazolidine-2,4-dione | |
| 149 | 4-(3-chlorophenyl)-1,3-bis(4-chlorophenyl)-5-hydroxy-4- | A |
| | methylimidazolidin-2-one | |
| 149-1 | (4S)-4-(3-chlorophenyl)-1,3-bis(4-chlorophenyl)-5-hydroxy-4- | B |
| | methylimidazolidin-2-one | |
| 149-2 | (4R)-4-(3-chlorophenyl)-1,3-bis(4-chlorophenyl)-5-hydroxy-4- | A |
| | methylimidazolidin-2-one | |
| 150 | 5,7-bis(4-chlorophenyl)-8-hydroxy-8-(3- | A |
| | (trifluoromethoxy)phenyl)-2-oxa-5,7-diazaspiro[3.4]octan-6-one | |
| 151 | 3-(5,7-bis(4-chlorophenyl)-8-hydroxy-6-oxo-2-oxa-5,7- | A |
| | diazaspiro[3.4]octan-8-yl)benzonitrile | |
| 152 | 1,3-bis(4-bromophenyl)-5-butyl-5-hydroxyimidazolidine-2,4-dione | D |
| 153 | 5-hydroxy-5-(4-isopropylphenyl)-1,3-diphenylimidazolidine-2,4- | NA |
| | dione | |
| 154 | 1,3,5-tris(4-chlorophenyl)-5-hydroxyimidazolidine-2,4-dione | D |
| 155 | 1,3-bis(4-chloro-2-fluorophenyl)-5-hydroxy-5- | NA |
| | phenylimidazolidine-2,4-dione | |
| 156 | 5-(4-cyclopropoxyphenyl)-5-hydroxy-1,3-diphenylimidazolidine- | NA |
| | 2,4-dione | |
| 157 | 1,3-bis(4-bromophenyl)-5-hexyl-5-hydroxyimidazolidine-2,4-dione | D |
| 158 | 3-(1,3-bis(4-chlorophenyl)-4-hydroxy-5,5-dimethyl-2- | A |
| | oxoimidazolidin-4-yl)-5-bromobenzonitrile | |
| 159 | 1,3-bis(4-chlorophenyl)-4-hydroxy-5-methyl-4-(3- | A |
| | (trifluoromethoxy)phenyl)-5-(trifluoromethyl)imidazolidin-2-one | |
| 159-1 | (5S)-1,3-bis(4-chlorophenyl)-4-hydroxy-5-methyl-4-(3- | B |
| | (trifluoromethoxy)phenyl)-5-(trifluoromethyl)imidazolidin-2-one | |
| 159-2 | (5R)-1,3-bis(4-chlorophenyl)-4-hydroxy-5-methyl-4-(3- | A |
| | (trifluoromethoxy)phenyl)-5-(trifluoromethyl)imidazolidin-2-one | |
| 160 | 5-hydroxy-1,3-bis(4-methoxyphenyl)-5-phenylimidazolidine-2,4- | NA |
| | dione | |
| 161 | 1,3-bis(4-fluorophenyl)-5-hydroxy-5-phenylimidazolidine-2,4- | NA |
| | dione | |
| 162 | 1,3-bis(3-chlorophenyl)-5-hydroxy-5-phenylimidazolidine-2,4- | C |
| | dione | |
| 163 | 1,3-bis(4-chlorophenyl)-4-ethyl-5-hydroxy-4-methyl-5-(3- | A |
| | (trifluoromethoxy)phenyl)imidazolidin-2-one | |
| 163-1 | (4R)-1,3-bis(4-chlorophenyl)-4-ethyl-5-hydroxy-4-methyl-5-(3- | B |
| | (trifluoromethoxy)phenyl)imidazolidin-2-one | |
| 163-2 | (4S)-1,3-bis(4-chlorophenyl)-4-ethyl-5-hydroxy-4-methyl-5-(3- | A |
| | (trifluoromethoxy)phenyl)imidazolidin-2-one | |
| 164 | 1,3-bis(4-bromophenyl)-5-(4-cyclopropoxyphenyl)-5- | NA |
| | hydroxyimidazolidine-2,4-dione | |
| 165 | 5,7-bis(4-chlorophenyl)-8-(3-(difluoromethoxy)phenyl)-8-hydroxy- | A |
| | 5,7-diazaspiro[3.4]octan-6-one | |
| 166 | 1,3-bis(4-chlorophenyl)-5-((dimethylamino)methyl)-4-hydroxy-4- | A |
| | (3-(trifluoromethoxy)phenyl)imidazolidin-2-one | |
| 166-1 | (5S)-1,3-bis(4-chlorophenyl)-5-((dimethylamino)methyl)-4- | A |
| | hydroxy-4-(3-(trifluoromethoxy)phenyl)imidazolidin-2-one | |
| 166-2 | (5R)-1,3-bis(4-chlorophenyl)-5-((dimethylamino)methyl)-4- | A |
| | hydroxy-4-(3-(trifluoromethoxy)phenyl)imidazolidin-2-one | |
| 167 | 1,3-bis(4-bromophenyl)-5-(4-(cyclopentyloxy)phenyl)-5- | D |
| | hydroxyimidazolidine-2,4-dione | |
| 168 | 1,3-bis(4-(cyclopentyloxy)phenyl)-5-hydroxy-5- | NA |
| | phenylimidazolidine-2,4-dione | |
| 169 | 1,3-bis(4-bromophenyl)-5-(4-(cyclohexyloxy)phenyl)-5- | NA |
| | hydroxyimidazolidine-2,4-dione | |
| 170 | 1,3-bis(4-chlorophenyl)-4-hydroxy-5-methyl-5-propyl-4-(3- | A |
| | (trifluoromethoxy)phenyl)imidazolidin-2-one | |
| 170-1 | (5R)-1,3-bis(4-chlorophenyl)-4-hydroxy-5-methyl-5-propyl-4-(3- | C |
| | (trifluoromethoxy)phenyl)imidazolidin-2-one | |
| 170-2 | (5S)-1,3-bis(4-chlorophenyl)-4-hydroxy-5-methyl-5-propyl-4-(3- | A |
| | (trifluoromethoxy)phenyl)imidazolidin-2-one | |
| 171 | 1,3-bis(4-bromophenyl)-5-(3-chloro-4-cyclopropoxyphenyl)-5- | D |
| | hydroxyimidazolidine-2,4-dione | |
| 172 | 1,3-bis(4-bromophenyl)-5-(4-fluorophenyl)-5- | C |
| | hydroxyimidazolidine-2,4-dione | |
| 173 | 1,3-bis(4-bromophenyl)-5-hydroxy-5-(4-(piperidin-1- | NA |
| | yl)phenyl)imidazolidine-2,4-dione | |
| 174 | 1,3-bis(4-bromophenyl)-5-(4-cyclobutoxyphenyl)-5- | NA |
| | hydroxyimidazolidine-2,4-dione | |
| 176 | 4-hydroxy-5,5-dimethyl-1,3-di-p-tolyl-4-(3- | A |
| | (trifluoromethoxy)phenyl)imidazolidin-2-one | |
| 177 | 3-(1,3-bis(4-chlorophenyl)-4-hydroxy-5-methyl-2-oxo-5- | A |
| | (trifluoromethyl)imidazolidin-4-yl)benzonitrile | |
| 177-1 | 3-((5S)-1,3-bis(4-chlorophenyl)-4-hydroxy-5-methyl-2-oxo-5- | A |
| | (trifluoromethyl)imidazolidin-4-yl)benzonitrile | |
| 177-2 | 3-((5R)-1,3-bis(4-chlorophenyl)-4-hydroxy-5-methyl-2-oxo-5- | B |
| | (trifluoromethyl)imidazolidin-4-yl)benzonitrile | |
| 178 | 1,3-bis(4-bromophenyl)-5-(4′-fluoro-[1,1′-biphenyl]-4-yl)-5- | NA |
| | hydroxyimidazolidine-2,4-dione | |
| 179 | 1,3-bis(4-bromophenyl)-5-hydroxy-5-(3- | C |
| | methoxyphenyl)imidazolidine-2,4-dione | |
| 180 | 4,4′-(4-hydroxy-5-methyl-2-oxo-4-(3- | B |
| | (trifluoromethoxy)phenyl)imidazolidine-1,3-diyl)dibenzonitrile | |
| 180-1 | 4,4′-((5S)-4-hydroxy-5-methyl-2-oxo-4-(3- | A |
| | (trifluoromethoxy)phenyl)imidazolidine-1,3-diyl)dibenzonitrile | |
| 180-2 | 4,4′-((5R)-4-hydroxy-5-methyl-2-oxo-4-(3- | A |
| | (trifluoromethoxy)phenyl)imidazolidine-1,3-diyl)dibenzonitrile | |
| 181 | 1,3-bis(3-bromophenyl)-5-hydroxy-5-phenylimidazolidine-2,4- | D |
| | dione | |
| 182 | 1,3-bis(4-bromophenyl)-5-(4-butoxyphenyl)-5- | NA |
| | hydroxyimidazolidine-2,4-dione | |
| 183 | 1,3-bis(4-bromophenyl)-5-(4-(cyclopentylmethoxy)phenyl)-5- | NA |
| | hydroxyimidazolidine-2,4-dione | |
| 184 | 1,3-bis(4-bromophenyl)-5-(4-(cyclopropylmethoxy)phenyl)-5- | NA |
| | hydroxyimidazolidine-2,4-dione | |
| 185-1 | 3-((5R)-1,3-bis(4-chlorophenyl)-5-ethyl-4-hydroxy-5-methyl-2- | C |
| | oxoimidazolidin-4-yl)benzonitrile | |
| 185-2 | 3-((5S)-1,3-bis(4-chlorophenyl)-5-ethyl-4-hydroxy-5-methyl-2- | A |
| | oxoimidazolidin-4-yl)benzonitrile | |
| 186 | 5-(1,3-bis(4-chlorophenyl)-4-hydroxy-5,5-dimethyl-2- | A |
| | oxoimidazolidin-4-yl)nicotinonitrile | |
| 187-1 | 3-((5S)-1,3-bis(4-chlorophenyl)-5-cyclopropyl-4-hydroxy-2- | A |
| | oxoimidazolidin-4-yl)benzonitrile | |
| 187-2 | 3-((5R)-1,3-bis(4-chlorophenyl)-5-cyclopropyl-4-hydroxy-2- | A |
| | oxoimidazolidin-4-yl)benzonitrile | |
| 188 | 5-(1,3-bis(4-chlorophenyl)-4-hydroxy-5,5-dimethyl-2- | A |
| | oxoimidazolidin-4-yl)isophthalonitrile | |
| 189 | 1,3-bis(4-chlorophenyl)-4-hydroxy-5,5-dimethyl-4-(pyridin-3- | A |
| | yl)imidazolidin-2-one | |
| 190 | 1,3-bis(4-chlorophenyl)-5-hydroxy-4-methyl-4-(3- | A |
| | (trifluoromethyl)phenyl)imidazolidin-2-one | |
| 190-1 | (4S)-1,3-bis(4-chlorophenyl)-5-hydroxy-4-methyl-4-(3- | NA |
| | (trifluoromethyl)phenyl)imidazolidin-2-one | |
| 190-2 | (4R)-1,3-bis(4-chlorophenyl)-5-hydroxy-4-methyl-4-(3- | A |
| | (trifluoromethyl)phenyl)imidazolidin-2-one | |
| 191 | 1,3-bis(4-chlorophenyl)-5-cyclobutyl-4-hydroxy-4-(3- | A |
| | (trifluoromethoxy)phenyl)imidazolidin-2-one | |
| 191-1 | (5S)-1,3-bis(4-chlorophenyl)-5-cyclobutyl-4-hydroxy-4-(3- | A |
| | (trifluoromethoxy)phenyl)imidazolidin-2-one | |
| 191-2 | (5R)-1,3-bis(4-chlorophenyl)-5-cyclobutyl-4-hydroxy-4-(3- | A |
| | (trifluoromethoxy)phenyl)imidazolidin-2-one | |
| 192 | 3-(4-hydroxy-5,5-dimethyl-2-oxo-1,3-bis(4- | A |
| | (trifluoromethyl)phenyl)imidazolidin-4-yl)benzonitrile | |
| 193 | 1,3-bis(4-bromophenyl)-5-hydroxy-5-methylimidazolidine-2,4- | D |
| | dione | |
| 194 | 3-(1,3-bis(4-chlorophenyl)-5-cyclobutyl-4-hydroxy-2- | A |
| | oxoimidazolidin-4-yl)benzonitrile | |
| 194-1 | 3-((5S)-1,3-bis(4-chlorophenyl)-5-cyclobutyl-4-hydroxy-2- | A |
| | oxoimidazolidin-4-yl)benzonitrile | |
| 194-2 | 3-((5R)-1,3-bis(4-chlorophenyl)-5-cyclobutyl-4-hydroxy-2- | A |
| | oxoimidazolidin-4-yl)benzonitrile | |
| 195 | 1,3-bis(4-bromophenyl)-5-hydroxy-5-(naphthalen-1- | NA |
| | yl)imidazolidine-2,4-dione | |
| 196 | 4-hydroxy-5-isopropyl-4-(3-(trifluoromethoxy)phenyl)-1,3-bis(4- | A |
| | (trifluoromethyl)phenyl)imidazolidin-2-one | |
| 196-1 | (5S)-4-hydroxy-5-isopropyl-4-(3-(trifluoromethoxy)phenyl)-1,3- | A |
| | bis(4-(trifluoromethyl)phenyl)imidazolidin-2-one | |
| 196-2 | (5R)-4-hydroxy-5-isopropyl-4-(3-(trifluoromethoxy)phenyl)-1,3- | A |
| | bis(4-(trifluoromethyl)phenyl)imidazolidin-2-one | |
| 197 | 1,3-bis(4-bromophenyl)-5-(5-chlorothiophen-2-yl)-5- | C |
| | hydroxyimidazolidine-2,4-dione | |
| 198 | 1,3-bis(4-bromophenyl)-5-(furan-2-yl)-5-hydroxyimidazolidine- | NA |
| | 2,4-dione | |
| 199 | 1,3-bis(4-bromophenyl)-5-hydroxy-5-octylimidazolidine-2,4-dione | NA |
| 200 | 1,3-bis(4-bromophenyl)-5-hydroxy-5-(p-tolyl)imidazolidine-2,4- | D |
| | dione | |
| 201 | 1,3-bis(4-bromophenyl)-5-hydroxy-5-(thiazol-2-yl)imidazolidine- | NA |
| | 2,4-dione | |
| 202 | 1,3-bis(4-bromophenyl)-5-hydroxy-5-(6-methoxypyridin-3- | C |
| | yl)imidazolidine-2,4-dione | |
| 203 | 5-hydroxy-1,3-bis(4-iodophenyl)-5-phenylimidazolidine-2,4-dione | C |
| 205 | 1,3-bis(4-bromophenyl)-5-hydroxy-5-(thiophen-2-yl)imidazolidine- | D |
| | 2,4-dione | |
| 206 | 1,3-bis(4-bromophenyl)-5-hydroxy-5-(2- | NA |
| | methoxyphenyl)imidazolidine-2,4-dione | |
| 207 | 1,3-bis(4-bromophenyl)-5-hydroxy-5-(tetrahydro-2H-pyran-4- | C |
| | yl)imidazolidine-2,4-dione | |
| 208 | 1,3-bis(4-bromophenyl)-5-hydroxy-5-(5-methoxypyridin-2- | D |
| | yl)imidazolidine-2,4-dione | |
| 209-1 | (4S)-1,3-bis(4-bromophenyl)-4-cyclopropyl-5-hydroxy-4- | C |
| | phenylimidazolidin-2-one | |
| 209-2 | (4R)-1,3-bis(4-bromophenyl)-4-cyclopropyl-5-hydroxy-4- | A |
| | phenylimidazolidin-2-one | |
| 210 | 1,3-bis(4-bromophenyl)-5-hydroxy-5-(1H-indol-2- | C |
| | yl)imidazolidine-2,4-dione | |
| 211 | 1,3-bis(4-bromophenyl)-5-hydroxy-5-(1H-pyrrol-2- | C |
| | yl)imidazolidine-2,4-dione | |
| 212 | 3-(8-hydroxy-6-oxo-5,7-bis(4-(trifluoromethyl)phenyl)-5,7- | A |
| | diazaspiro[3.4]octan-8-yl)benzonitrile | |
| 213 | 5-(benzofuran-2-yl)-1,3-bis(4-bromophenyl)-5- | NA |
| | hydroxyimidazolidine-2,4-dione | |
| 214 | 5-(benzo[b]thiophen-2-yl)-1,3-bis(4-bromophenyl)-5- | C |
| | hydroxyimidazolidine-2,4-dione | |
| 215 | 1,3-bis(4-bromophenyl)-5-(2,3-dihydro-1H-inden-2-yl)-5- | D |
| | hydroxyimidazolidine-2,4-dione | |
| 216 | 1,3-bis(4-bromophenyl)-5-hydroxy-5- | NA |
| | (trifluoromethyl)imidazolidine-2,4-dione | |
| 217 | ethyl 1,3-bis(4-chlorophenyl)-4-hydroxy-2-oxo-5- | B |
| | phenylimidazolidine-4-carboxylate | |
| 218 | 1,3-bis(4-chlorophenyl)-4-hydroxy-4-(hydroxymethyl)-5- | C |
| | phenylimidazolidin-2-one | |
| 218-1 | (4S,5R)-1,3-bis(4-chlorophenyl)-4-hydroxy-4-(hydroxymethyl)-5- | C |
| | phenylimidazolidin-2-one | |
| 218-2 | (4R,5S)-1,3-bis(4-chlorophenyl)-4-hydroxy-4-(hydroxymethyl)-5- | C |
| | phenylimidazolidin-2-one | |
| 219-1 | (4S)-1,3-bis(4-bromophenyl)-5-hydroxy-4-methyl-4-(3- | C |
| | (trifluoromethyl)phenyl)imidazolidin-2-one | |
| 219-2 | (4R)-1,3-bis(4-bromophenyl)-5-hydroxy-4-methyl-4-(3- | A |
| | (trifluoromethyl)phenyl)imidazolidin-2-one | |
| 220 | 1,3-bis(4-chlorophenyl)-5-hydroxy-4-isopropyl-4-(3- | A |
| | (trifluoromethoxy)phenyl)imidazolidin-2-one | |
| 221 | 3-(1,3-bis(4-chlorophenyl)-4-hydroxy-2-oxo-7-oxa-1,3- | A |
| | diazaspiro[4.4]nonan-4-yl)benzonitrile | |
| 221-2 | 3-((4R)-1,3-bis(4-chlorophenyl)-4-hydroxy-2-oxo-7-oxa-1,3- | A |
| | diazaspiro[4.4]nonan-4-yl)benzonitrile | |
| 221-1 | 3-((4S)-1,3-bis(4-chlorophenyl)-4-hydroxy-2-oxo-7-oxa-1,3- | A |
| | diazaspiro[4.4]nonan-4-yl)benzonitrile | |
| 222 | 1,3-bis(4-chlorophenyl)-5-hydroxy-4-methyl-4-(m- | A |
| | tolyl)imidazolidin-2-one | |
| 222-1 | (4S)-1,3-bis(4-chlorophenyl)-5-hydroxy-4-methyl-4-(m- | C |
| | tolyl)imidazolidin-2-one | |
| 222-2 | (4R)-1,3-bis(4-chlorophenyl)-5-hydroxy-4-methyl-4-(m- | A |
| | tolyl)imidazolidin-2-one | |
| 223 | 1,3-bis(4-chlorophenyl)-4-hydroxy-5-methyl-4- | A |
| | phenylimidazolidin-2-one | |
| 223-1 | (4S)-1,3-bis(4-chlorophenyl)-5-hydroxy-4-methyl-4- | C |
| | phenylimidazolidin-2-one | |
| 223-2 | (4R)-1,3-bis(4-chlorophenyl)-5-hydroxy-4-methyl-4- | A |
| | phenylimidazolidin-2-one | |
| 224 | 4,6-bis(4-chlorophenyl)-7-hydroxy-7-(3- | A |
| | (trifluoromethoxy)phenyl)-4,6-diazaspiro[2.4]heptan-5-one | |
| 225 | 4,6-bis(4-chlorophenyl)-7-hydroxy-7-(3-(trifluoromethyl)phenyl)- | A |
| | 4,6-diazaspiro[2.4]heptan-5-one | |
| 226 | 3-(4,6-bis(4-chlorophenyl)-7-hydroxy-5-oxo-4,6- | A |
| | diazaspiro[2.4]heptan-7-yl)benzonitrile | |
| 227 | 4,6-bis(4-chlorophenyl)-7-hydroxy-7-(3-hydroxyphenyl)-4,6- | B |
| | diazaspiro[2.4]heptan-5-one | |
| 228 | 7-(3-chlorophenyl)-4,6-bis(4-chlorophenyl)-7-hydroxy-4,6- | A |
| | diazaspiro[2.4]heptan-5-one | |
| 229 | 3-(4-bromophenyl)-4-hydroxy-5-methyl-1-phenyl-4-(3- | A |
| | (trifluoromethoxy)phenyl)imidazolidin-2-one | |
| 230 | 4-hydroxy-5-methyl-1,3-diphenyl-4-(3- | B |
| | (trifluoromethoxy)phenyl)imidazolidin-2-one | |
| 230-1 | (5S)-4-hydroxy-5-methyl-1,3-diphenyl-4-(3- | B |
| | (trifluoromethoxy)phenyl)imidazolidin-2-one | |
| 230-2 | (5R)-4-hydroxy-5-methyl-1,3-diphenyl-4-(3- | B |
| | (trifluoromethoxy)phenyl)imidazolidin-2-one | |
| 231 | 3-(1,3-bis(4-chlorophenyl)-5-hydroxy-4-isopropyl-2- | B |
| | oxoimidazolidin-4-yl)benzonitrile | |
| 232 | 3-(1,3-bis(4-bromophenyl)-5-hydroxy-4-isopropyl-2- | B |
| | oxoimidazolidin-4-yl)benzonitrile | |
| 233 | 1,3-bis(4-chlorophenyl)-5-cyclopropyl-4-(3- | A |
| | (difluoromethoxy)phenyl)-4-hydroxyimidazolidin-2-one | |
| 233-1 | (5S)-1,3-bis(4-chlorophenyl)-5-cyclopropyl-4-(3- | A |
| | (difluoromethoxy)phenyl)-4-hydroxyimidazolidin-2-one | |
| 233-2 | (5R)-1,3-bis(4-chlorophenyl)-5-cyclopropyl-4-(3- | A |
| | (difluoromethoxy)phenyl)-4-hydroxyimidazolidin-2-one | |
| 234 | 1,3-bis(4-chlorophenyl)-4-hydroxy-4-(3- | A |
| | (trifluoromethoxy)phenyl)-7-oxa-1,3-diazaspiro[4.4]nonan-2-one | |
| 234-1 | (4R,5S)-1,3-bis(4-chlorophenyl)-4-hydroxy-4-(3- | A |
| | (trifluoromethoxy)phenyl)-7-oxa-1,3-diazaspiro[4.4]nonan-2-one | |
| 234-2 | (4S,5S)-1,3-bis(4-chlorophenyl)-4-hydroxy-4-(3- | A |
| | (trifluoromethoxy)phenyl)-7-oxa-1,3-diazaspiro[4.4]nonan-2-one | |
| 234-3 | (4R,5R)-1,3-bis(4-chlorophenyl)-4-hydroxy-4-(3- | A |
| | (trifluoromethoxy)phenyl)-7-oxa-1,3-diazaspiro[4.4]nonan-2-one | |
| 234-4 | (4S,5R)-1,3-bis(4-chlorophenyl)-4-hydroxy-4-(3- | A |
| | (trifluoromethoxy)phenyl)-7-oxa-1,3-diazaspiro[4.4]nonan-2-one | |
| 235 | 4-hydroxy-5-isopropyl-1,3-di-p-tolyl-4-(3- | A |
| | (trifluoromethoxy)phenyl)imidazolidin-2-one | |
| 235-1 | (5S)-4-hydroxy-5-isopropyl-1,3-di-p-tolyl-4-(3- | A |
| | (trifluoromethoxy)phenyl)imidazolidin-2-one | |
| 235-2 | (5R)-4-hydroxy-5-isopropyl-1,3-di-p-tolyl-4-(3- | A |
| | (trifluoromethoxy)phenyl)imidazolidin-2-one | |
| 236 | 4,6-bis(4-chlorophenyl)-7-(3-(difluoromethoxy)phenyl)-7-hydroxy- | A |
| | 4,6-diazaspiro[2.4]heptan-5-one | |
| 237 | 1,3-bis(4-chlorophenyl)-4-hydroxy-5-(oxetan-3-yl)-4-(3- | A |
| | (trifluoromethoxy)phenyl)imidazolidin-2-one | |
| 238-1 | (5S)-1,3-bis(4-chlorophenyl)-4-(3-(difluoromethoxy)phenyl)-4- | A |
| | hydroxy-5-(methoxymethyl)imidazolidin-2-one | |
| 238-2 | (5R)-1,3-bis(4-chlorophenyl)-4-(3-(difluoromethoxy)phenyl)-4- | A |
| | hydroxy-5-(methoxymethyl)imidazolidin-2-one | |
| 239 | 8-hydroxy-5,7-di-p-tolyl-8-(3-(trifluoromethoxy)phenyl)-5,7- | A |
| | diazaspiro[3.4]octan-6-one | |
| 240 | 5,7-bis(4-chlorophenyl)-2-fluoro-8-hydroxy-8-(3- | A |
| | (trifluoromethoxy)phenyl)-5,7-diazaspiro[3.4]octan-6-one | |
| 241 | 4-hydroxy-1,3-bis(4-methoxyphenyl)-5,5-dimethyl-4-(3- | A |
| | (trifluoromethoxy)phenyl)imidazolidin-2-one | |
| 242 | 5,7-bis(4-chlorophenyl)-8-hydroxy-8-(3- | A |
| | (trifluoromethoxy)phenyl)-5,7-diazaspiro[3.4]octan-6-one | |
| 243 | 1,3-bis(4-chlorophenyl)-4-propyl-5-(3-(trifluoromethyl)phenyl)- | B |
| | 1,3-dihydro-2H-imidazol-2-one | |
| 244 | 1,3-bis(4-chlorophenyl)-4-(3-ethylphenyl)-5-propyl-1,3-dihydro- | B |
| | 2H-imidazol-2-one | |
| 245 | 1,3-bis(4-chlorophenyl)-4-propyl-5-(3-(trifluoromethoxy)phenyl)- | A |
| | 1,3-dihydro-2H-imidazol-2-one | |
| 246 | 4-(3-bromophenyl)-1,3-bis(4-chlorophenyl)-5-methyl-1,3-dihydro- | A |
| | 2H-imidazol-2-one | |
| 247 | 1,3-bis(4-chlorophenyl)-4-ethyl-5-(3-(trifluoromethoxy)phenyl)- | A |
| | 1,3-dihydro-2H-imidazol-2-one | |
| 248 | 1,3-bis(4-bromophenyl)-4-propyl-5-(3-(trifluoromethoxy)phenyl)- | A |
| | 1,3-dihydro-2H-imidazol-2-one | |
| 249 | 1,3-bis(4-bromophenyl)-4-cyclopropyl-5-phenyl-1,3-dihydro-2H- | A |
| | imidazol-2-one | |
| 250 | 1,3-bis(4-bromophenyl)-4-(3-chlorophenyl)-5-propyl-1,3-dihydro- | A |
| | 2H-imidazol-2-one | |
| 251 | 1,3-bis(4-chlorophenyl)-4,5-dipropyl-1,3-dihydro-2H-imidazol-2- | NA |
| | one | |
| 252 | 3-(5-ethyl-1,3-bis(4-fluorophenyl)-2-oxo-2,3-dihydro-1H-imidazol- | B |
| | 4-yl)benzonitrile | |
| 253 | 4-(3-acetylphenyl)-1,3-bis(4-chlorophenyl)-5-methyl-1,3-dihydro- | B |
| | 2H-imidazol-2-one | |
| 254 | 4-(3-chlorophenyl)-1,3-bis(4-chlorophenyl)-5-(tetrahydrofuran-2- | B |
| | yl)-1,3-dihydro-2H-imidazol-2-one | |
| 256 | 1,3-bis(4-bromophenyl)-4-(1H-pyrrol-2-yl)-1,3-dihydro-2H- | NA |
| | imidazol-2-one | |
| 257 | 1,3-bis(4-bromophenyl)-4-methyl-5-phenyl-1,3-dihydro-2H- | A |
| | imidazol-2-one | |
| 258 | 3-(1,3-bis(4-chlorophenyl)-5-ethyl-2-oxo-2,3-dihydro-1H- | A |
| | imidazol-4-yl)benzonitrile | |
| 259 | 1,3-bis(4-chlorophenyl)-4-(m-tolyl)-1,3-dihydro-2H-imidazol-2- | NA |
| | one | |
| 260 | 1,3-bis(4-chlorophenyl)-4-methyl-5-(m-tolyl)-1,3-dihydro-2H- | B |
| | imidazol-2-one | |
| 261 | 1,3-bis(4-bromophenyl)-4-phenyl-5-propyl-1,3-dihydro-2H- | B |
| | imidazol-2-one | |
| 262 | 1,3-bis(4-bromophenyl)-4-methyl-5-(3-(trifluoromethoxy)phenyl)- | B |
| | 1,3-dihydro-2H-imidazol-2-one | |
| 263 | 1,3-bis(4-bromophenyl)-4-methyl-5-(m-tolyl)-1,3-dihydro-2H- | B |
| | imidazol-2-one | |
| 264 | 1,3-bis(4-bromophenyl)-4-ethyl-5-phenyl-1,3-dihydro-2H- | A |
| | imidazol-2-one | |
| 265 | 3-(1,3-bis(4-chlorophenyl)-2-oxo-2,3-dihydro-1H-imidazol-4-yl)-5- | NA |
| | chlorobenzonitrile | |
| 266 | 1,3-bis(4-bromophenyl)-4-methyl-5-(4-methylthiophen-2-yl)-1,3- | B |
| | dihydro-2H-imidazol-2-one | |
| 267 | 1,3-bis(4-bromophenyl)-4-isopropyl-5-phenyl-1,3-dihydro-2H- | A |
| | imidazol-2-one | |
| 268 | 3-(7-(4-chlorophenyl)-5-(4-cyanophenyl)-8-hydroxy-6-oxo-5,7- | A |
| | diazaspiro[3.4]octan-8-yl)benzonitrile | |
| 269 | 4-(7-(4-chlorophenyl)-8-hydroxy-6-oxo-8-(3- | A |
| | (trifluoromethoxy)phenyl)-5,7-diazaspiro[3.4]octan-5- | |
| | yl)benzonitrile | |
| 270 | 3-(1-(4-cyanophenyl)-4-hydroxy-5,5-dimethyl-2-oxo-3-(p- | A |
| | tolyl)imidazolidin-4-yl)benzonitrile | |
| 271 | 5,7-bis(4-chlorophenyl)-8-(3-(1,1-difluoroethyl)phenyl)-8-hydroxy- | A |
| | 2-oxa-5,7-diazaspiro[3.4]octan-6-one | |
| 272 | 5,7-bis(4-chlorophenyl)-8-(3-(difluoromethoxy)phenyl)-8-hydroxy- | A |
| | 2-oxa-5,7-diazaspiro[3.4]octan-6-one | |
| 272B | 5,7-bis(4-chlorophenyl)-8-hydroxy-8-(3-hydroxyphenyl)-2-oxa- | NT |
| | 5,7-diazaspiro[3.4]octan-6-one | |
| 273 | 4-(3-(4-chlorophenyl)-5-hydroxy-4,4-dimethyl-2-oxo-5-(3- | A |
| | (trifluoromethoxy)phenyl)imidazolidin-1-yl)benzonitrile | |
| 274 | 4-(5-(4-chlorophenyl)-8-hydroxy-6-oxo-8-(3- | A |
| | (trifluoromethoxy)phenyl)-2-oxa-5,7-diazaspiro[3.4]octan-7- | |
| | yl)benzonitrile | |
| 275 | 4,4′-(8-hydroxy-6-oxo-8-(3-(trifluoromethoxy)phenyl)-2-oxa-5,7- | A |
| | diazaspiro[3.4]octane-5,7-diyl)dibenzonitrile | |
| 276 | 4-(7-(4-chlorophenyl)-8-hydroxy-6-oxo-8-(3- | A |
| | (trifluoromethoxy)phenyl)-2-oxa-5,7-diazaspiro[3.4]octan-5- | |
| | yl)benzonitrile | |
| 277 | 4-(8-hydroxy-6-oxo-7-(p-tolyl)-8-(3-(trifluoromethoxy)phenyl)-2- | A |
| | oxa-5,7-diazaspiro[3.4]octan-5-yl)benzonitrile | |
| 278 | 3-(5-(4-cyanophenyl)-8-hydroxy-6-oxo-7-(p-tolyl)-2-oxa-5,7- | A |
| | diazaspiro[3.4]octan-8-yl)benzonitrile | |
| 279 | 4-(8-hydroxy-6-oxo-7-(p-tolyl)-8-(3-(trifluoromethoxy)phenyl)- | A |
| | 5,7-diazaspiro[3.4[octan-5-yl)benzonitrile | |
| 280 | 3-(5-(4-cyanophenyl)-8-hydroxy-6-oxo-7-(p-tolyl)-5,7- | A |
| | diazaspiro[3.4]octan-8-yl)benzonitrile | |
| 281 | 4-(8-hydroxy-6-oxo-5-(p-tolyl)-8-(3-(trifluoromethoxy)phenyl)- | A |
| | 5,7-diazaspiro[3.4]octan-7-yl)benzonitrile | |
| 282 | 3-(7-(4-cyanophenyl)-8-hydroxy-6-oxo-5-(p-tolyl)-5,7- | A |
| | diazaspiro[3.4]octan-8-yl)benzonitrile | |
| 283 | 3-(7-(4-chlorophenyl)-8-hydroxy-6-oxo-5-(p-tolyl)-2-oxa-5,7- | A |
| | diazaspiro[3.4]octan-8-yl)benzonitrile | |
| 284 | 8-hydroxy-5,7-di-p-tolyl-8-(3-(trifluoromethoxy)phenyl)-2-oxa- | A |
| | 5,7-diazaspiro[3.4]octan-6-one | |
| 285 | 8-(3-(difluoromethyl)phenyl)-8-hydroxy-5,7-di-p-tolyl-2-oxa-5,7- | A |
| | diazaspiro[3.4]octan-6-one | |
| 286 | 8-(3-(1,1-difluoroethyl)phenyl)-8-hydroxy-5,7-di-p-tolyl-2-oxa- | A |
| | 5,7-diazaspiro[3.4]octan-6-one | |
| 287 | 3-(8-hydroxy-6-oxo-5,7-di-p-tolyl-2-oxa-5,7-diazaspiro[3.4]octan- | A |
| | 8-yl)benzonitrile | |
| 288 | 4-(5-(4-chlorophenyl)-8-hydroxy-6-oxo-8-(3- | A |
| | (trifluoromethoxy)phenyl)-5,7-diazaspiro[3.4]octan-7- | |
| | yl)benzonitrile | |
| 289 | 3-(5-(4-chlorophenyl)-7-(4-cyanophenyl)-8-hydroxy-6-oxo-5,7- | A |
| | diazaspiro[3.4]octan-8-yl)benzonitrile | |
| 290 | 8-(3-(difluoromethoxy)phenyl)-8-hydroxy-5,7-di-p-tolyl-2-oxa-5,7- | A |
| | diazaspiro[3.4]octan-6-one | |
| 290B | 8-hydroxy-8-(3-hydroxyphenyl)-5,7-di-p-tolyl-2-oxa-5,7- | NT |
| | diazaspiro[3.4]octan-6-one | |
| 291 | 3-(1,3-bis(4-chlorophenyl)-4-hydroxy-5-methyl-2-oxo-5- | A |
| | propylimidazolidin-4-yl)benzonitrile | |
| 291A | 1,3-bis(4-chlorophenyl)-4-hydroxy-4-(3-iodophenyl)-5-methyl-5- | NT |
| | propylimidazolidin-2-one | |
| 292 | 4-(7-(4-bromophenyl)-8-hydroxy-6-oxo-8-(3- | A |
| | (trifluoromethoxy)phenyl)-5,7-diazaspiro[3.4]octan-5- | |
| | yl)benzonitrile | |
| 293 | 4,4′-(8-hydroxy-6-oxo-8-(3-(trifluoromethoxy)phenyl)-5,7- | A |
| | diazaspiro[3.4]octane-5,7-diyl)dibenzonitrile | |
| 294 | 4,4′-(8-(3-cyanophenyl)-8-hydroxy-6-oxo-5,7- | A |
| | diazaspiro[3.4]octane-5,7-diyl)dibenzonitrile | |
| 295 | 3-(7-(4-bromophenyl)-5-(4-cyanophenyl)-8-hydroxy-6-oxo-5,7- | A |
| | diazaspiro[3.4]octan-8-yl)benzonitrile | |
| 296 | 1,3-bis(4-chlorophenyl)-4-hydroxy-5-(methoxymethyl)-5-methyl- | A |
| | 4-(3-(trifluoromethoxy)phenyl)imidazolidin-2-one | |
| 296-1 | (4R,5R)-1,3-bis(4-chlorophenyl)-4-hydroxy-5-(methoxymethyl)-5- | |
| | methyl-4-(3-(trifluoromethoxy)phenyl)imidazolidin-2-one | B |
| 296-2 | (4S,5S)-1,3-bis(4-chlorophenyl)-4-hydroxy-5-(methoxymethyl)-5- | A |
| | methyl-4-(3-(trifluoromethoxy)phenyl)imidazolidin-2-one | |
| 296-3 | (4R,5S)-1,3-bis(4-chlorophenyl)-4-hydroxy-5-(methoxymethyl)-5- | B |
| | methyl-4-(3-(trifluoromethoxy)phenyl)imidazolidin-2-one | |
| 296-4 | (4S,5R)-1,3-bis(4-chlorophenyl)-4-hydroxy-5-(methoxymethyl)-5- | A |
| | methyl-4-(3-(trifluoromethoxy)phenyl)imidazolidin-2-one | |
| 297 | 3-(1,3-bis(4-chlorophenyl)-4-hydroxy-5-(methoxymethyl)-5- | A |
| | methyl-2-oxoimidazolidin-4-yl)benzonitrile | |
| 297-1 | 3-((5R)-1,3-bis(4-chlorophenyl)-4-hydroxy-5-(methoxymethyl)-5- | A |
| | methyl-2-oxoimidazolidin-4-yl)benzonitrile | |
| 297-2 | 3-((5S)-1,3-bis(4-chlorophenyl)-4-hydroxy-5-(methoxymethyl)-5- | A |
| | methyl-2-oxoimidazolidin-4-yl)benzonitrile | |
| 298 | 3-(5-(4-chlorophenyl)-8-hydroxy-6-oxo-7-(p-tolyl)-2-oxa-5,7- | A |
| | diazaspiro[3.4]octan-8-yl)benzonitrile | |
| 299 | 5-(4-chlorophenyl)-8-hydroxy-7-(p-tolyl)-8-(3- | A |
| | (trifluoromethoxy)phenyl)-2-oxa-5,7-diazaspiro[3.4]octan-6-one | |
| 300 | 3-(5,7-bis(4-chlorophenyl)-8-hydroxy-6-oxo-2,5,7- | B |
| | triazaspiro[3.4]octan-8-yl)benzonitrile 2,2,2-trifluoroacetate | |
| 301 | 3-(5,7-bis(4-chlorophenyl)-8-hydroxy-2-methyl-6-oxo-2,5,7- | A |
| | triazaspiro[3.4]octan-8-yl)benzonitrile 2,2,2-trifluoroacetate | |
| 302 | 8-hydroxy-8-(3-(trifluoromethoxy)phenyl)-5,7-bis(4- | A |
| | (trifluoromethyl)phenyl)-2-oxa-5,7-diazaspiro[3.4]octan-6-one | |
| 303 | 3-(8-hydroxy-6-oxo-5,7-bis(4-(trifluoromethyl)phenyl)-2-oxa-5,7- | A |
| | diazaspiro[3.4]octan-8-yl)benzonitrile | |
| 304 | 5,7-bis(4-chlorophenyl)-2,2-difluoro-8-hydroxy-8-(3- | A |
| | (trifluoromethoxy)phenyl)-5,7-diazaspiro | |
| 305 | 3-(5,7-bis(4-chlorophenyl)-2,2-difluoro-8-hydroxy-6-oxo-5,7- | A |
| | diazaspiro[3.4]octan-8-yl)benzonitrile | |
| 305A | 5,7-bis(4-chlorophenyl)-2,2-difluoro-8-hydroxy-8-(3-iodophenyl)- | NT |
| | 5,7-diazaspiro[3.4]octan-6-one | |
| 306 | 5-(5,7-bis(4-chlorophenyl)-8-hydroxy-6-oxo-2-oxa-5,7- | A |
| | diazaspiro[3.4]octan-8-yl)isophthalonitrile | |
| 306H | 5,7-bis(4-chlorophenyl)-8-(3,5-dibromophenyl)-8-hydroxy-2-oxa- | NT |
| | 5,7-diazaspiro[3.4]octan-6-one | |
| 307 | 4-(8-hydroxy-6-oxo-5-(p-tolyl)-8-(3-(trifluoromethoxy)phenyl)-2- | A |
| | oxa-5,7-diazaspiro[3.4]octan-7-yl)benzonitrile | |
| 308 | 3-(7-(4-cyanophenyl)-8-hydroxy-6-oxo-5-(p-tolyl)-2-oxa-5,7- | A |
| | diazaspiro[3.4]octan-8-yl)benzonitrile | |
| 309 | 3-(3-(4-chlorophenyl)-1-(4-cyanophenyl)-4-hydroxy-5,5-dimethyl- | A |
| | 2-oxoimidazolidin-4-yl)benzonitrile | |
| 310 | 8-(3-bromothiophen-2-yl)-5,7-bis(4-chlorophenyl)-8-hydroxy-2- | A |
| | oxa-5,7-diazaspiro[3.4]octan-6-one | |
| 311 | 7-(4-chlorophenyl)-8-hydroxy-5-(p-tolyl)-8-(3- | A |
| | (trifluoromethoxy)phenyl)-2-oxa-5,7-diazaspiro[3.4]octan-6-one | |
| 312 | 4-(4-hydroxy-5,5-dimethyl-2-oxo-3-(p-tolyl)-4-(3- | A |
| | (trifluoromethoxy)phenyl)imidazolidin-1-yl)benzonitrile | |
| 313 | 3-(3-(4-cyanophenyl)-4-hydroxy-5,5-dimethyl-2-oxo-1-(p- | A |
| | tolyl)imidazolidin-4-yl)benzonitrile | |
| 314 | 4-(5-hydroxy-4,4-dimethyl-2-oxo-3-(p-tolyl)-5-(3- | A |
| | (trifluoromethoxy)phenyl)imidazolidin-1-yl)benzonitrile | |
| 315 | 7-(4-chlorophenyl)-8-hydroxy-5-(p-tolyl)-8-(3- | A |
| | (trifluoromethoxy)phenyl)-5,7-diazaspiro[3.4]octan-6-one | |
| 316 | 3-(7-(4-chlorophenyl)-8-hydroxy-6-oxo-5-(p-tolyl)-5,7- | A |
| | diazaspiro[3.4]octan-8-yl)benzonitrile | |
| 317 | 3-(3-(4-chlorophenyl)-4-hydroxy-5,5-dimethyl-2-oxo-1-(p- | A |
| | tolyl)imidazolidin-4-yl)benzonitrile | |
| 318 | 1-(4-chlorophenyl)-5-hydroxy-4,4-dimethyl-3-(p-tolyl)-5-(3- | A |
| | (trifluoromethoxy)phenyl)imidazolidin-2-one | |
| 319 | 4,4′-(4-ethyl-5-hydroxy-4-methyl-2-oxo-5-(3- | NT |
| | (trifluoromethoxy)phenyl)imidazolidine-1,3-diyl)dibenzonitrile | |
| 319-1 | 4,4′-((4R)-4-ethyl-5-hydroxy-4-methyl-2-oxo-5-(3- | A |
| | (trifluoromethoxy)phenyl)imidazolidine-1,3-diyl)dibenzonitrile | |
| 319-2 | 4′-((4S)-4-ethyl-5-hydroxy-4-methyl-2-oxo-5-(3- | A |
| | (trifluoromethoxy)phenyl)imidazolidine-1,3-diyl)dibenzonitrile | |
| 320 | 4,4'-(4-hydroxy-5-methyl-2-oxo-5-propyl-4-(3- | NT |
| | (trifluoromethoxy)phenyl)imidazolidine-1,3-diyl)dibenzonitrile | |
| 320-1 | 4,4′-((5R)-4-hydroxy-5-methyl-2-oxo-5-propyl-4-(3- | NT |
| | (trifluoromethoxy)phenyl)imidazolidine-1,3-diyl)dibenzonitrile | |
| 320-2 | 4,4′-((5S)-4-hydroxy-5-methyl-2-oxo-5-propyl-4-(3- | A |
| | (trifluoromethoxy)phenyl)imidazolidine-1,3-diyl)dibenzonitrile | |
| 321 | 3-(1,3-bis(4-chlorophenyl)-5,5-diethyl-4-hydroxy-2- | A |
| | oxoimidazolidin-4-yl)benzonitrile | |
| 321E | 1,3-bis(4-chlorophenyl)-4,4-diethyl-5-hydroxy-5-(3- | NT |
| | iodophenyl)imidazolidin-2-one | |
| 322 | 4-(3-(4-chlorophenyl)-4-hydroxy-5,5-dimethyl-2-oxo-4-(3- | A |
| | (trifluoromethoxy)phenyl)imidazolidin-1-yl)benzonitrile | |
| 323 | 3-(1-(4-chlorophenyl)-3-(4-cyanophenyl)-4-hydroxy-5,5-dimethyl- | A |
| | 2-oxoimidazolidin-4-yl)benzonitrile | |
| 324-1 | 1,3-bis(4-chlorophenyl)-4-hydroxy-5,5-dimethyl-4-(3-(oxetan-2- | A |
| | yl)phenyl)imidazolidin-2-one | |
| 324-2 | 1,3-bis(4-chlorophenyl)-4-hydroxy-5,5-dimethyl-4-(3-(oxetan-3- | B |
| | yl)phenyl)imidazolidin-2-one | |
| 324A | 1,3-bis(4-chlorophenyl)-4-hydroxy-5,5-dimethyl-4-(3- | NT |
| | vinylphenyl)imidazolidin-2-one | |
| 324B | 1,3-bis(4-chlorophenyl)-4-hydroxy-5,5-dimethyl-4-(3-(oxiran-2- | NT |
| | yl)phenyl)imidazolidin-2-one | |
| 325 | 4-(3-(4-bromophenyl)-4-hydroxy-2-oxo-4-(3- | A |
| | (trifluoromethoxy)phenyl)-1,3-diazaspiro[4.4]nonan-1- | |
| | yl)benzonitrile | |
| 326 | 4,4′-(4-hydroxy-2-oxo-4-(3-(trifluoromethoxy)phenyl)-1,3- | A |
| | diazaspiro[4.4]nonane-1,3-diyl)dibenzonitrile | |
| 327 | 4-hydroxy-1,3-di-p-tolyl-4-(3-(trifluoromethoxy)phenyl)-1,3- | A |
| | diazaspiro[4.4]nonan-2-one | |
| 328 | 3-(4-hydroxy-1,3-bis(4-methoxyphenyl)-5,5-dimethyl-2- | B |
| | oxoimidazolidin-4-yl)benzonitrile | |
| 329 | 4-hydroxy-5-isopropyl-1,3-bis(4-methoxyphenyl)-4-(3- | A |
| | (trifluoromethoxy)phenyl)imidazolidin-2-one | |
| 329-1 | (5S)-4-hydroxy-5-isopropyl-1,3-bis(4-methoxyphenyl)-4-(3- | B |
| | (trifluoromethoxy)phenyl)imidazolidin-2-one | |
| 329-2 | (5R)-4-hydroxy-5-isopropyl-1,3-bis(4-methoxyphenyl)-4-(3- | A |
| | (trifluoromethoxy)phenyl)imidazolidin-2-one | |
| 330 | 3-(4-hydroxy-5-isopropyl-1,3-bis(4-methoxyphenyl)-2- | A |
| | oxoimidazolidin-4-yl)benzonitrile | |
| 331 | 5-(1,3-bis(4-chlorophenyl)-4-hydroxy-5-methyl-2-oxo-5- | A |
| | (trifluoromethyl)imidazolidin-4-yl)isophthalonitrile | |
| 331-1 | 5-((5S)-1,3-bis(4-chlorophenyl)-4-hydroxy-5-methyl-2-oxo-5- | A |
| | (trifluoromethyl)imidazolidin-4-yl)isophthalonitrile | |
| 331-2 | 5-((5R)-1,3-bis(4-chlorophenyl)-4-hydroxy-5-methyl-2-oxo-5- | A |
| | (trifluoromethyl)imidazolidin-4-yl)isophthalonitrile | |
| 331A | 1,3-bis(4-chlorophenyl)-4-(3,5-dibromophenyl)-4-hydroxy-5- | NT |
| | methyl-5-(trifluoromethyl)imidazolidin-2-one | |
| 332 | 5-(4-chlorophenyl)-8-hydroxy-7-phenyl-8-(3- | A |
| | (trifluoromethoxy)phenyl)-5,7-diazaspiro[3.4]octan-6-one | |
| 332A | 7-(4-bromophenyl)-5-(4-chlorophenyl)-8-hydroxy-8-(3- | NT |
| | (trifluoromethoxy)phenyl)-5,7-diazaspiro[3.4]octan-6-one | |
| 333 | 5-(4-chlorophenyl)-8-hydroxy-7-(p-tolyl)-8-(3- | A |
| | (trifluoromethoxy)phenyl)-5,7-diazaspiro[3.4]octan-6-one | |
| 334 | 7-(4-chlorophenyl)-8-hydroxy-5-phenyl-8-(3- | A |
| | (trifluoromethoxy)phenyl)-5,7-diazaspiro[3.4]octan-6-one | |
| 335 | 4-(8-hydroxy-6-oxo-5-phenyl-8-(3-(trifluoromethoxy)phenyl)-5,7- | A |
| | diazaspiro[3.4]octan-7-yl)benzonitrile | |
| 336 | 4-(8-hydroxy-6-oxo-7-phenyl-8-(3-(trifluoromethoxy)phenyl)-5,7- | A |
| | diazaspiro[3.4]octan-5-yl)benzonitrile | |
| 336B | 4-(7-(4-bromophenyl)-8-hydroxy-6-oxo-8-(3- | NT |
| | (trifluoromethoxy)phenyl)-5,7-diazaspiro[3.4]octan-5- | |
| | yl)benzonitrile | |
| 337 | 4-(7-(4-ethylphenyl)-8-hydroxy-6-oxo-8-(3- | A |
| | (trifluoromethoxy)phenyl)-2-oxa-5,7-diazaspiro[3.4]octan-5- | |
| | yl)benzonitrile | |
| 338 | 5-(4-chlorophenyl)-8-hydroxy-7-phenyl-8-(3- | NT |
| | (trifluoromethoxy)phenyl)-2-oxa-5,7-diazaspiro[3.4]octan-6-one | |

### Biological Example 2

### CGT Enzyme Assay

Full-length human CGT cDNA was cloned into the BamH1/Xho1 site of pcDNA3.1 (+) mammalian expression vector (V90-20, Invitrogen, Carlsbad, Calif.) and the plasmid was transfected into Chinese hamster ovary (CHO) cells. Lysate was prepared using M-PER (Mammalian Protein Extraction Reagent, ThermoFisher Scientific, Grand Island, N.Y.) in the presence of a protease inhibitor cocktail (P8340, Sigma, Saint Louis, Mo.). Each 100 mm dish (100% confluent, approximately 1×10⁷ cells) was lysed with 250 μL of M-PER containing protease inhibitors. Protein concentration was determined using a Pierce BCA protein assay kit (ThermoFisher Scientific). Four micrograms of CHO/CGT lysate was incubated with various concentrations of a compound (0.001 μM-50 μM) in 10 mM HEPES (pH 7.2) containing 35 μM dioleoylphosphatidylcholine, 5 mM MgCl₂, 5 mM MnCl₂, 1% BSA, 15 mM KCl, 1 mM EGTA, 8 mM CHAPS, 0.01% tween-80, 10 μM C6-NBD-dihydro-ceramide and 17.5 μM UDP-galactose in a final reaction volume of 20 μL at 37° C. for 1 hour. The final concentration of DMSO was 0.5% in both compound-treated and mock-treated samples. Each individual reaction was diluted with 80 μL of methanol:acetronitrile (1:3) containing 5 μM N-docdecanoyl-NBD-galactosylceramide (internal standard) to stop the reaction. 200 μL H₂O:acetonitile (1:1) was added to precipitate the protein. After sufficient mixing, the plates were centrifuged at 2469 g for 38 minutes, 200 μl of supernatant was transferred to a LC-MS 96-deep well plate and a second spin was performed at 2469 g for 10 minutes. The final supernatant was injected in triplicate for RapidFire/MS/MS analysis.

Quantitative Analysis Using the RapidFire/MS/MS Method:

The quantitative analysis of C6-NBD-dihydro-ceramide and C6-NBD-dihydro-galactosylceramide was performed on a Rapid Fire 360 high-throughput mass spectrometry system (Agilent Technologies, Palo Alto, Calif.) coupled with a API4000⁺ triple quadrupole mass spectrometer (Applied Biosystems, Concord, Ontario, Canada). RapidFire software packages, including RapidFire Control panel, RapidFire UI and RapidFire Integrator (Agilent Technologies), were used to control the RapidFire instrument and to process data. Analyst 1.6.2 software packages (Applied Biosystems) were used to control the MS system and acquire MS data. 10 μL of sample was loaded on a micro-scale C4 solid-phase extraction (SPE) cartridge (Agilent Technologies) and salts were removed using water supplemented with 0.1% formic acid at the flow rate of 1.5 mL/min for 3 sec. C6-NBD-dihydro-ceramide and C6-NBD-dihydro-galactosylceramide were co-eluted into the mass spectrometer using acetonitrile containing 0.1% formic acid at the flow rate of 1.0 mL/min for 3 sec. The total cycle time of one injection was 8 secs. The MS/MS detection was performed in ESI negative mode. The mass transition of C6-NBD-dihydro-galactosylceramide was m/z 752.6→678.6 using a −40 V collision energy, the mass transition of C6-NBD-dihydro-ceramide was m/z 590.6→115.8 using a −40 V collision energy and the mass transition of N-docdecanoyl-NBD-galactosylceramide was m/z 820.9→746.3 using a −45 V collision energy.

The C6-NBD-dihydro-galactosylceramide reading was normalized first by dividing the peak area of C6-NBD-dihydro-galactosylceramide by the peak area of the internal standard, N-docdecanoyl-NBD-galactosylceramide. IC₅₀ values (see Table 1, data ranges correspond to analysis using the RapidFire/MS/MS method with 0.01% Tween 80 added) were generated from sigmoidal dose-response curves (variable slope) with GraphPad Prism software (GraphPad Software, Inc., San Diego, Calif.) using the normalized peak areas of C6-NBD-dihydro-galactosylceramide or the percent inhibition of C6-NBD-dihydro-galactosylceramide accumulation relative to DMSO control.

An alternative RapidFire/MS/MS method can be used to generate IC₅₀ values. The method is identical to the one described above except that 0.01% tween is not used.

An alternative LC-MS/MS based method can be used. For the LC-MS/MS readout, the CGT assay is identical to the one described above except for the following: 10 μg of CHO/CGT lysate can be used as the CGT enzyme source, C12:0 (2R—OH) ceramide can be used as the ceramide substrate and 0.3 μM C12 ceramide can be used as the internal standard. Tween 80 is not present in the reaction mixture. The final supernatant can be injected for LC-MS/MS analysis.

Quantitative Analysis Using the LC-MS/MS Method:

The quantitative analysis of C12:0 (2R—OH) ceramide and C12:0 (2R—OH) galactosylceramide by LC-MS/MS can be performed on a Shimadzu ultra-fast liquid chromatography (Shimadzu, Japan) coupled with API 4000 mass spectrometer (Applied Biosystems, Concord, Ontario, Canada). Analyst 1.5 software packages (Applied Biosystems) can be used to control the LC-MS/MS system, as well as for data acquisition and processing. 10 μL of sample can be loaded onto a Luna C18 column (50 mm×2.0 mm, I.D. 3 μm, 100A) (Phenomenex, USA) for chromatographic separation. Mobile phase A can consist of HPLC grade water with 0.1% formic acid (v/v) and mobile phase B can consist of acetonitrile supplemented with 0.1% formic acid (v/v). The separation can be achieved using the following gradient program at a flow rate of 0.8 mL/min: the initial mobile phase can be 70% B, which can be increased in a linear fashion to 95% B in 1.50 min, and then maintained at 95% B until 3.50 min. The mobile phase can then be reset to 70% B within 0.01 min, and maintained until 4.50 min. The total run time can be 4.50 min. The MS/MS detection can be performed in ESI positive mode. The mass transition selected for quantification was can be m/z 498.5-264.3 for C12:0 (2R—OH) ceramide under the collision energy of 38.8 V, and the mass transition of C12:0 (2R—OH) galactosylceramide can be m/z 660.6→264.5 under the collision energy of 51 V. The mass transition of C12 ceramide used as internal standard can be m/z 482.4-264.3 under the collision energy of 33V. Compound IC₅₀ values can be calculated using a protocol identical to the one described for the RapidFire method.

### CGT Cellular Assay

A stable cell line overexpressing human full-length CGT was made by transfecting Chinese hamster ovary (CHO) cells with 10 μg hCGT-pcDNA3.1(+) DNA and 30 μL of X-tremeGENE HP DNA transfection reagent (6366236001, Roche, Indianapolis, Ind.), followed by 800 μg/mL G418 selection. CHO cells stably expressing human full-length CGT (B5 cells) were seeded onto a CytoOne 96-well TC plate (USA Scientific, CC7682-7596) at 3×10⁴ cells/well in F12K media containing 10% FBS and incubated overnight at 37° C., 5% CO₂. The next day, the media was removed and replaced with 90 μL incubation media (F12K media containing 5% FBS and 1.1 μM eliglustat). In a separate 96-well tissue culture plate, the test compounds were serially diluted in DMSO (1 mM to 0.1 μM) followed by a 100-fold dilution with F12K media containing 5% FBS. 10 μL of compound was added to the cells and incubated at 37° C., 5% CO₂ for 2 hours. The final concentration of DMSO was 0.1% in both compound-treated and mock-treated cells. The C6-NBD-dihydro-ceramide substrate was diluted with F12K medium containing 5% FBS and 11% BSA to make a 110 μM substrate solution. 10 μL of substrate solution was added to the plate and incubated at 37° C., 5% CO₂ for 1 hour. Following the reaction, the plate was washed two times with PBS followed by the addition of 120 μL of lipid extraction solvent (methanol with 0.5% acetic acid) containing 1 μM of N-dodecanoyl-NBD-galactosylceramide (internal standard). The sealed plate was placed on shaker for 2 hours (around 20 RPM) to extract product. After centrifugation at 2469 g for 30 mins, 80 μL of supernatant was transferred to a LC/MS 96-deep well plate containing 40 μL ddH₂O. After mixing thoroughly, a second spin was performed at 2469 g for 20 mins. The final supernatant was injected for LC-MS/MS analysis.

The quantitative analysis of C6-NBD-dihydro-ceramide and C6-NBD-dihydro-galactosylceramide was performed on a Shimadzu ultra-fast liquid chromatography (Shimadzu, Japan) coupled with API 4000 mass spectrometer (Applied Biosystems, Concord, Ontario, Canada). Analyst 1.5 software packages (Applied Biosystems) were used to control the LC-MS/MS system, as well as for data acquisition and processing. 10 μL of sample was loaded onto a KinetexC18 column (50 mm×2.1 mm, I.D.2.6 μm, 100A) (Phenomenex, USA) for chromatographic separation. Mobile phase A consisted of HPLC grade water with 0.1% formic acid (v/v), and mobile phase B consisted of acetonitrile supplemented with 0.1% formic acid (v/v). The separation was achieved using the following gradient program at a flow rate of 0.7 mL/min: the initial mobile phase was 35% B and was ramped in a linear fashion to 98% B in 2.40 min. From 2.40 to 3.00 min, the gradient was maintained at 98% B. Then, mobile phase was reset to 35% B in 0.01 min, and maintained until 3.50 min. The total run time was 3.50 mins. The MS/MS detection was performed in ESI negative mode. The mass transition of C6-NBD-dihydro-ceramide was m/z 590.4-516.4 under the collision energy of −34 V. The mass transition of C6-NBD-dihydro-galactosylceramide was m/z 752.4-678.5 under the collision energy of −46 V. The mass transition of N-Dodecanoyl-NBD-galactosylceramide which was used as internal standard was m/z 820.5-115.6 under the collision energy of −72V. Compound IC₅₀ values were calculated using a protocol identical to the ones described above for the CGT enzyme assay.

Using the above CGT enzyme and CGT cellular assays, the compounds of Table 2 were tested.

In Table 2, biological data range of IC₅₀ values calculated from the enzymatic assays (using the RapidFire/MS/MS method with 0.01% Tween 80 added) are provided, where:

A is <1 M;

B is ≥1 to 10 M;

C is >10 to 30 M;

D is >30 to 100 M; and

NT is not tested.

TABLE 2
Compounds of Formula (VII)

| Example | | IC₅₀ |
|---|---|---|
| No. | Name | (μM) |
| 1 | 1,3-bis(4-bromophenyl)octahydro-2H- | D |
| | benzo[d]imidazol-2-one | |
| 2-3 | (4S,5R)-1,3-bis(4-bromophenyl)-4-methyl-5- | B |
| | phenylimidazolidin-2-one | |
| 2-4 | (4R,5S)-1,3-bis(4-bromophenyl)-4-methyl-5- | NT |
| | phenylimidazolidin-2-one | |
| 2-1 | (4S,5S)-1,3-bis(4-bromophenyl)-4-methyl-5- | D |
| | phenylimidazolidin-2-one | |
| 2-2 | (4R,5R)-1,3-bis(4-bromophenyl)-4-methyl-5- | A |
| | phenylimidazolidin-2-one | |
| 3 | 1,3-bis(4-chlorophenyl)-4-methyl-5-(m- | D |
| | tolyl)imidazolidin-2-one | |
| 4-1 | (4S,5S)-1,3-bis(4-chlorophenyl)-4-methyl-5-(3- | C |
| | (trifluoromethoxy)phenyl)imidazolidin-2-one | |
| 4-2 | (4R,5R)-1,3-bis(4-chlorophenyl)-4-methyl-5-(3- | C |
| | (trifluoromethoxy)phenyl)imidazolidin-2-one | |
| 5-1 | 3-((4R,5R)-1,3-bis(4-chlorophenyl)-5-methyl-2- | C |
| | oxoimidazolidin-4-yl)benzonitrile | |
| 5-2 | 3-((4S,5S)-1,3-bis(4-chlorophenyl)-5-methyl-2- | C |
| | oxoimidazolidin-4-yl)benzonitrile | |
| 6 | 1,3-bis(4-chlorophenyl)-4,4-dimethyl-5-(3- | A |
| | (trifluoromethyl)phenyl)imidazolidin-2-one | |
| 7 | 1,3-bis(4-chlorophenyl)-4,4-dimethyl-5-(3- | A |
| | (trifluoromethoxy)phenyl)imidazolidin-2-one | |
| 8 | 5-(3-chlorophenyl)-1,3-bis(4-chlorophenyl)-4,4- | A |
| | dimethylimidazolidin-2-one | |
| 9 | 1,3-bis(4-chlorophenyl)-5-(3- | A |
| | (difluoromethoxy)phenyl)-4,4-dimethylimidazolidin- | |
| | 2-one | |
| 10 | 3-(1,3-bis(4-chlorophenyl)-5,5-dimethyl-2- | A |
| | oxoimidazolidin-4-yl)benzonitrile | |
| 8-1 | (R)-5-(3-chlorophenyl)-1,3-bis(4-chlorophenyl)-4,4- | C |
| | dimethylimidazolidin-2-one | |
| 8-2 | (S)-5-(3-chlorophenyl)-1,3-bis(4-chlorophenyl)-4,4- | A |
| | dimethylimidazolidin-2-one | |
| 10-1 | (R)-3-(1,3-bis(4-chlorophenyl)-5,5-dimethyl-2- | C |
| | oxoimidazolidin-4-yl)benzonitrile | |
| 10-2 | (S)-3-(1,3-bis(4-chlorophenyl)-5,5-dimethyl-2- | A |
| | oxoimidazolidin-4-yl)benzonitrile | |
| 7-1 | (R)-1,3-bis(4-chlorophenyl)-4,4-dimethyl-5-(3- | B |
| | (trifluoromethoxy)phenyl)imidazolidin-2-one | |
| 7-2 | (S)-1,3-bis(4-chlorophenyl)-4,4-dimethyl-5-(3- | A |
| | (trifluoromethoxy)phenyl)imidazolidin-2-one | |
| 6-1 | (R)-1,3-bis(4-chlorophenyl)-4,4-dimethyl-5-(3- | C |
| | (trifluoromethyl)phenyl)imidazolidin-2-one | |
| 6-2 | (S)-1,3-bis(4-chlorophenyl)-4,4-dimethyl-5-(3- | A |
| | (trifluoromethyl)phenyl)imidazolidin-2-one | |
| 9-1 | (R)-1,3-bis(4-chlorophenyl)-5-(3- | C |
| | (difluoromethoxy)phenyl)-4,4-dimethylimidazolidin- | |
| | 2-one | |
| 9-2 | (S)-1,3-bis(4-chlorophenyl)-5-(3- | A |
| | (difluoromethoxy)phenyl)-4,4-dimethylimidazolidin- | |
| | 2-one | |
| 11-1 | (4R,5R)-1,3-bis(4-bromophenyl)-4-methyl-5-(3- | B |
| | (trifluoromethoxy)phenyl)imidazolidin-2-one | |
| 11-2 | (4S,5S)-1,3-bis(4-bromophenyl)-4-methyl-5-(3- | A |
| | (trifluoromethoxy)phenyl)imidazolidin-2-one | |
| 12 | 1,3-bis(4-chlorophenyl)-3a-(3- | B |
| | (trifluoromethoxy)phenyl) | |
| | hexahydrocyclopenta[d]imidazol-2(1H)-one | |
| 12-1 | (3aS,6aS)-1,3-bis(4-chlorophenyl)- | B |
| | 3a-(3-(trifluoromethoxy) | |
| | phenyl)hexahydrocyclopenta[d]imidazol-2(1H)-one | |
| 12-2 | (3aR,6aR)-1,3-bis(4-chlorophenyl)-3a-(3- | C |
| | (trifluoromethoxy) | |
| | phenyl)hexahydrocyclopenta[d]imidazol-2(1H)-one | |
| 13-1 | (3aS,6aS)-1,3-bis(4-chlorophenyl)-3a- | C |
| | (3-(trifluoromethyl)phenyl) | |
| | hexahydrocyclopenta[d]imidazol-2(1H)-one | |
| 13-2 | (3aR,6aR)-1,3-bis(4-chlorophenyl)-3a-(3- | C |
| | (trifluoromethyl) | |
| | phenyl)hexahydrocyclopenta[d]imidazol-2(1H)-one | |
| 14 | 3-(1,3-bis(4-chlorophenyl)-2-oxooctahydro-3aH- | A |
| | benzo[d]imidazol-3a-yl)benzonitrile | |
| 15-1 | 1,3-bis(4-chlorophenyl)-3a-(3- | C |
| | (trifluoromethoxy)phenyl)octahydro-2H- | |
| | benzo[d]imidazol-2-one | |
| 16-1 | (3aS,6aS)-1,3-bis(4-chlorophenyl)-3a- | C |
| | (3-(difluoromethyl)phenyl) | |
| | hexahydrocyclopenta[d]imidazol-2(1H)-one | |
| 16-2 | (3aR,6aR)-1,3-bis(4-chlorophenyl)-3a-(3- | C |
| | (difluoromethyl) | |
| | phenyl)hexahydrocyclopenta[d]imidazol-2(1H)-one | |
| 17 | 1,3-bis(4-chlorophenyl)-4,4-diethyl-5-(3- | A |
| | (trifluoromethoxy) phenyl)imidazolidin-2-one | |
| 18 | 1,3-bis(4-chlorophenyl)-3a-(3-hydroxyphenyl) | C |
| | hexahydrocyclopenta[d]imidazol-2(1H)-one | |
| 15-2 | (R)-1,3-bis(4-chlorophenyl)-4-(3- | A |
| | (trifluoromethoxy)phenyl)-1,3-diazaspiro[4.4]nonan- | |
| | 2-one | |
| 15-3 | (S)-1,3-bis(4-chlorophenyl)-4-(3- | C |
| | (trifluoromethoxy)phenyl)-1,3-diazaspiro[4.4]nonan- | |
| | 2-one | |
| 19-1 | (4R,5R)-1,3-bis(4-chlorophenyl)-4-ethyl-4-methyl-5- | B |
| | (3-(trifluoromethoxy)phenyl)imidazolidin-2-one | |
| 19-2 | (4R,5S)-1,3-bis(4-chlorophenyl)-4-ethyl-4-methyl-5- | A |
| | (3-(trifluoromethoxy)phenyl)imidazolidin-2-one | |
| 19-3 | (4S,5S)-1,3-bis(4-chlorophenyl)-4-ethyl-4-methyl-5- | A |
| | (3-(trifluoromethoxy)phenyl)imidazolidin-2-one | |
| 19-4 | (4S,5R)-1,3-bis(4-chlorophenyl)-4-ethyl-4-methyl-5- | B |
| | (3-(trifluoromethoxy)phenyl)imidazolidin-2-one | |
| 20-1 | (R)-4,4-dimethyl-5-(3-(trifluoromethoxy)phenyl)-1,3- | A |
| | bis(4-(trifluoromethyl)phenyl)imidazolidin-2-one | |
| 20-2 | (S)-4,4-dimethyl-5-(3-(trifluoromethoxy)phenyl)-1,3- | C |
| | bis(4-(trifluoromethyl)phenyl)imidazolidin-2-one | |
| 21-1 | (R)-4,4-dimethyl-1,3-di-p-tolyl-5-(3- | B |
| | (trifluoromethoxy)phenyl)imidazolidin-2-one | |
| 21-2 | (S)-4,4-dimethyl-1,3-di-p-tolyl-5-(3- | A |
| | (trifluoromethoxy)phenyl)imidazolidin-2-one | |
| 22-2 | (4R,5R)-1,3-bis(4-chlorophenyl)-4-methyl-4-propyl- | A |
| | 5-(3-(trifluoromethoxy)phenyl)imidazolidin-2-one | |
| 22-3 | (4R,5S)-1,3-bis(4-chlorophenyl)-4-methyl-4-propyl- | A |
| | 5-(3-(trifluoromethoxy)phenyl)imidazolidin-2-one | |
| 22-5 | (4S,5S)-1,3-bis(4-chlorophenyl)-4-methyl-4-propyl- | C |
| | 5-(3-(trifluoromethoxy)phenyl)imidazolidin-2-one | |
| 23-1 | (3aS,6aS)-1,3-bis(4-chlorophenyl)-3a-(3- | C |
| | (difluoromethoxy) | |
| | phenyl)hexahydrocyclopenta[d]imidazol-2(1H)-one | |
| 23-2 | (3aR,6aR)-1,3-bis(4-chlorophenyl)-3a-(3- | C |
| | (difluoromethoxy) | |
| | phenyl)hexahydrocyclopenta[d]imidazol-2(1H)-one | |
| 24-1 | (R)-3-(5,5-dimethyl-2-oxo-1,3-bis(4- | A |
| | (trifluoromethyl)phenyl)imidazolidin-4- | |
| | yl)benzonitrile | |
| 24-2 | (S)-3-(5,5-dimethyl-2-oxo-1,3-bis(4- | C |
| | (trifluoromethyl)phenyl)imidazolidin-4- | |
| | yl)benzonitrile | |
| 25-1 | (4S,5S)-1,3-bis(4-chlorophenyl)-4-methyl-5-(3- | A |
| | (trifluoromethoxy)phenyl)-4- | |
| | (trifluoromethyl)imidazolidin-2-one | |
| 25-2 | (4R,5R)-1,3-bis(4-chlorophenyl)-4-methyl-5-(3- | B |
| | (trifluoromethoxy)phenyl)-4- | |
| | (trifluoromethyl)imidazolidin-2-one | |
| 17-1 | (R)-1,3-bis(4-chlorophenyl)-4,4-diethyl-5-(3- | A |
| | (trifluoromethoxy)phenyl)imidazolidin-2-one | |
| 17-2 | (S)-1,3-bis(4-chlorophenyl)-4,4-diethyl-5-(3- | B |
| | (trifluoromethoxy)phenyl)imidazolidin-2-one | |
| 26-1 | (R)-4,4-dimethyl-1,3-diphenyl-5-(3- | C |
| | (trifluoromethoxy)phenyl)imidazolidin-2-one | |
| 26-2 | (S)-4,4-dimethyl-1,3-diphenyl-5-(3- | B |
| | (trifluoromethoxy)phenyl)imidazolidin-2-one | |
| 14-1 | 3-((3aR,7aR)-1,3-bis(4-chlorophenyl)-2- | B |
| | oxooctahydro-3aH-benzo[d]imidazol-3a- | |
| | yl)benzonitrile | |
| 14-2 | 3-((3aS,7aS)-1,3-bis(4-chlorophenyl)-2- | C |
| | oxooctahydro-3aH-benzo[d]imidazol-3a- | |
| | yl)benzonitrile | |
| 27-1 | 3-((4S,5S)-1,3-bis(4-chlorophenyl)-5-methyl-2-oxo- | C |
| | 5-(trifluoromethyl)imidazolidin-4-yl)benzonitrile | |
| 27-2 | 3-((4R,5R)-1,3-bis(4-chlorophenyl)-5-methyl-2-oxo- | A |
| | 5-(trifluoromethyl)imidazolidin-4-yl)benzonitrile | |
| 28-1 | (R)-1,3-bis(3-chlorophenyl)-4,4-dimethyl-5-(3- | C |
| | (trifluoromethoxy)phenyl)imidazolidin-2-one | |
| 28-2 | (S)-1,3-bis(3-chlorophenyl)-4,4-dimethyl-5-(3- | A |
| | (trifluoromethoxy)phenyl)imidazolidin-2-one | |
| 29-1 | (R)-5,7-bis(4-chlorophenyl)-8-(3- | B |
| | (trifluoromethoxy)phenyl)-2-oxa-5,7- | |
| | diazaspiro[3.4]octan-6-one | |
| 29-2 | (S)-5,7-bis(4-chlorophenyl)-8-(3- | A |
| | (trifluoromethoxy)phenyl)-2-oxa-5,7- | |
| | diazaspiro[3.4]octan-6-one | |
| 30-1 | (R)-3-(5,7-bis(4-chlorophenyl)-6-oxo-2-oxa-5,7- | C |
| | diazaspiro[3.4]octan-8-yl)benzonitrile | |
| 30-2 | (S)-3-(5,7-bis(4-chlorophenyl)-6-oxo-2-oxa-5,7- | A |
| | diazaspiro[3.4]octan-8-yl)benzonitrile | |
| 31-1 | (3aR)-1,3-bis(4-chlorophenyl)-3a-(3- | C |
| | (trifluoromethoxy) phenyl)hexahydropyrano[3,4- | |
| | d]imidazol-2(3H)-one | |
| 31-2 | (3aS)-1,3-bis(4-chlorophenyl)-3a-(3- | A |
| | (trifluoromethoxy) phenyl)hexahydropyrano[3,4- | |
| | d]imidazol-2(3H)-one | |
| 32-1 | (R)-3-(6-oxo-5,7-di-p-tolyl-2-oxa-5,7- | C |
| | diazaspiro[3.4]octan-8-yl)benzonitrile | |
| 32-2 | (S)-3-(6-oxo-5,7-di-p-tolyl-2-oxa-5,7- | A |
| | diazaspiro[3.4]octan-8-yl)benzonitrile | |
| 33-3 | 3-((4S,5R)-1,3-bis(4-chlorophenyl)-5-methyl-2-oxo- | B |
| | 5-propylimidazolidin-4-yl)benzonitrile | |
| 33-4 | 3-((4R,5S)-1,3-bis(4-chlorophenyl)-5-methyl-2-oxo- | A |
| | 5-propylimidazolidin-4-yl)benzonitrile | |
| 34-1 | 3-((4S,5S)-1,3-bis(4-chlorophenyl)-5-ethyl-5-methyl- | A |
| | 2-oxoimidazolidin-4-yl)benzonitrile | |
| 35-1 | (R)-1,3-di-p-tolyl-4-(3-(trifluoromethoxy)phenyl)-1,3- | B |
| | diazaspiro[4.4]nonan-2-one | |
| 35-2 | (S)-1,3-di-p-tolyl-4-(3-(trifluoromethoxy)phenyl)-1,3- | A |
| | diazaspiro[4.4]nonan-2-one | |
| 33-1 | 3-((4R,5R)-1,3-bis(4-chlorophenyl)-5-methyl-2-oxo- | A |
| | 5-propylimidazolidin-4-yl)benzonitrile | |
| 33-2 | 3-((4S,5S)-1,3-bis(4-chlorophenyl)-5-methyl-2-oxo-5- | B |
| | propylimidazolidin-4-yl)benzonitrile | |
| 34-2 | 3-((4R,5R)-1,3-bis(4-chlorophenyl)-5-ethyl-5-methyl- | B |
| | 2-oxoimidazolidin-4-yl)benzonitrile | |
| 36-1 | (R)-4,4′-(6-oxo-8-(3-(trifluoromethoxy)phenyl)-2- | C |
| | oxa-5,7-diazaspiro[3.4]octane-5,7-diyl)dibenzonitrile | |
| 36-2 | (S)-4,4′-(6-oxo-8-(3-(trifluoromethoxy)phenyl)-2-oxa- | A |
| | 5,7-diazaspiro[3.4]octane-5,7-diyl)dibenzonitrile | |
| 37-1 | (4R,5R)-1,3-bis(4-chlorophenyl)-4-(methoxymethyl)- | B |
| | 4-methyl-5-(3- | |
| | (trifluoromethoxy)phenyl)imidazolidin-2-one | |
| 37-2 | (4S,5S)-1,3-bis(4-chlorophenyl)-4-(methoxymethyl)- | A |
| | 4-methyl-5-(3- | |
| | (trifluoromethoxy)phenyl)imidazolidin-2-one | |
| 34-3 | 3-((4R,5S)-1,3-bis(4-chlorophenyl)-5-ethyl-5-methyl- | A |
| | 2-oxoimidazolidin-4-yl)benzonitrile | |
| 34-4 | 3-((4S,5R)-1,3-bis(4-chlorophenyl)-5-ethyl-5-methyl- | C |
| | 2-oxoimidazolidin-4-yl)benzonitrile | |
| 38-1 | 3-((4R,5R)-1,3-bis(4-chlorophenyl)-5- | B |
| | (methoxymethyl)-5-methyl-2-oxoimidazolidin-4- | |
| | yl)benzonitrile | |
| 38-2 | 3-((4S,5S)-1,3-bis(4-chlorophenyl)-5- | A |
| | (methoxymethyl)-5-methyl-2-oxoimidazolidin-4- | |
| | yl)benzonitrile | |
| 39-1 | 4,4′-((4S,5S)-4-methyl-2-oxo-5-(3- | A |
| | (trifluoromethoxy)phenyl)-4- | |
| | (trifluoromethyl)imidazolidine-1,3-diyl)dibenzonitrile | |
| 39-2 | 4,4′-((4R,5R)-4-methyl-2-oxo-5-(3- | C |
| | (trifluoromethoxy)phenyl)-4- | |
| | (trifluoromethyl)imidazolidine-1,3-diyl)dibenzonitrile | |
| 40-1 | (R)-4-(3-(4-chlorophenyl)-4,4-dimethyl-2-oxo-5-(3- | C |
| | (trifluoromethoxy)phenyl)imidazolidin-1- | |
| | yl)benzonitrile | |
| 40-2 | (S)-4-(3-(4-chlorophenyl)-4,4-dimethyl-2-oxo-5-(3- | A |
| | (trifluoromethoxy)phenyl)imidazolidin-1- | |
| | yl)benzonitrile | |
| 41-1 | (R)-4-(3-(4-chlorophenyl)-5,5-dimethyl-2-oxo-4-(3- | C |
| | (trifluoromethoxy)phenyl)imidazolidin-1- | |
| | yl)benzonitrile | |
| 41-2 | (S)-4-(3-(4-chlorophenyl)-5,5-dimethyl-2-oxo-4-(3- | A |
| | (trifluoromethoxy)phenyl)imidazolidin-1- | |
| | yl)benzonitrile | |
| 42-1 | (R)-4-(6-oxo-5-(p-tolyl)-8-(3- | A |
| | (trifluoromethoxy)phenyl)-2-oxa-5,7- | |
| | diazaspiro[3.4]octan-7-yl)benzonitrile | |
| 42-2 | (S)-4-(6-oxo-5-(p-tolyl)-8-(3- | C |
| | (trifluoromethoxy)phenyl)-2-oxa-5,7- | |
| | diazaspiro[3.4]octan-7-yl)benzonitrile | |
| 43-1 | (R)-4-(6-oxo-7-(p-tolyl)-8-(3- | B |
| | (trifluoromethoxy)phenyl)-2-oxa-5,7- | |
| | diazaspiro[3.4]octan-5-yl)benzonitrile | |
| 43-2 | (S)-4-(6-oxo-7-(p-tolyl)-8-(3- | A |
| | (trifluoromethoxy)phenyl)-2-oxa-5,7- | |
| | diazaspiro[3.4]octan-5-yl)benzonitrile | |
| 44-1 | 4-((4S,5S)-3-(4-bromophenyl)-5-methyl-2-oxo-4-(3- | B |
| | (trifluoro methoxy)phenyl)-5- | |
| | (trifluoromethyl)imidazolidin-1-yl)benzonitrile | |
| 44-2 | 4-((4R,5R)-3-(4-bromophenyl)-5-methyl-2-oxo-4-(3- | A |
| | (trifluoro methoxy)phenyl)-5- | |
| | (trifluoromethyl)imidazolidin-1-yl)benzonitrile | |
| 45-1 | (R)-3-(5,7-bis(4-chlorophenyl)-2,2-difluoro-6-oxo- | B |
| | 5,7-diazaspiro[3.4]octan-8-yl)benzonitrile | |
| 45-2 | (S)-3-(5,7-bis(4-chlorophenyl)-2,2-difluoro-6-oxo- | A |
| | 5,7-diazaspiro[3.4]octan-8-yl)benzonitrile | |
| 46-1 | (R)-4-(7-(4-chlorophenyl)-6-oxo-8-(3- | A |
| | (trifluoromethoxy)phenyl)-2-oxa-5,7- | |
| | diazaspiro[3.4]octan-5-yl)benzonitrile | |
| 46-2 | (S)-4-(7-(4-chlorophenyl)-6-oxo-8-(3- | B |
| | (trifluoromethoxy)phenyl)-2-oxa-5,7- | |
| | diazaspiro[3.4]octan-5-yl)benzonitrile | |
| 47-1 | (R)-4-(5-(4-chlorophenyl)-6-oxo-8-(3- | C |
| | (trifluoromethoxy)phenyl)-2-oxa-5,7- | |
| | diazaspiro[3.4]octan-7-yl)benzonitrile | |
| 47-2 | (S)-4-(5-(4-chlorophenyl)-6-oxo-8-(3- | A |
| | (trifluoromethoxy)phenyl)-2-oxa-5,7- | |
| | diazaspiro[3.4]octan-7-yl)benzonitrile | |
| 48-1 | (R)-3-(3-(4-cyanophenyl)-5,5-dimethyl-2-oxo-1-(p- | C |
| | tolyl)imidazolidin-4-yl)benzonitrile | |
| 48-2 | (S)-3-(3-(4-cyanophenyl)-5,5-dimethyl-2-oxo-1-(p- | A |
| | tolyl)imidazolidin-4-yl)benzonitrile | |
| 49-1 | (R)-7-(4-chlorophenyl)-5-(p-tolyl)-8-(3- | B |
| | (trifluoromethoxy)phenyl)-2-oxa-5,7- | |
| | diazaspiro[3.4]octan-6-one | |
| 49-2 | (S)-7-(4-chlorophenyl)-5-(p-tolyl)-8-(3- | A |
| | (trifluoromethoxy)phenyl)-2-oxa-5,7- | |
| | diazaspiro[3.4]octan-6-one | |
| 50-1 | (R)-4-(4,4-dimethyl-2-oxo-3-(p-tolyl)-5-(3- | A |
| | (trifluoromethoxy)phenyl)imidazolidin-1- | |
| | yl)benzonitrile | |
| 50-2 | (S)-4-(4,4-dimethyl-2-oxo-3-(p-tolyl)-5-(3- | C |
| | (trifluoromethoxy)phenyl)imidazolidin-1- | |
| | yl)benzonitrile | |
| 51-2 | 4,4′-((4R,5S)-4-ethyl-4-methyl-2-oxo-5-(3- | A |
| | (trifluoromethoxy)phenyl)imidazolidine-1,3- | |
| | diyl)dibenzonitrile | |
| 51-3 | 4,4′-((4R,5R)-4-ethyl-4-methyl-2-oxo-5-(3- | B |
| | (trifluoromethoxy)phenyl)imidazolidine-1,3- | |
| | diyl)dibenzonitrile | |
| 51-6 | 4,4′-((4S,5R)-4-ethyl-4-methyl-2-oxo-5-(3- | C |
| | (trifluoromethoxy)phenyl)imidazolidine-1,3- | |
| | diyl)dibenzonitrile | |
| 52-2 | 4,4′-((4R,5S)-4-methyl-2-oxo-4-propyl-5-(3- | A |
| | (trifluoromethoxy)phenyl)imidazolidine-1,3- | |
| | diyl)dibenzonitrile | |
| 52-3 | 4,4′-((4R,5R)-4-methyl-2-oxo-4-propyl-5-(3- | B |
| | (trifluoromethoxy)phenyl)imidazolidine-1,3- | |
| | diyl)dibenzonitrile | |
| 52-6 | 4,4′-((4S,5R)-4-methyl-2-oxo-4-propyl-5-(3- | C |
| | (trifluoromethoxy)phenyl)imidazolidine-1,3- | |
| | diyl)dibenzonitrile | |
| 53-1 | (R)-4-(5,5-dimethyl-2-oxo-3-(p-tolyl)-4-(3- | B |
| | (trifluoromethoxy)phenyl)imidazolidin-1- | |
| | yl)benzonitrile | |
| 53-2 | (S)-4-(5,5-dimethyl-2-oxo-3-(p-tolyl)-4-(3- | A |
| | (trifluoromethoxy)phenyl)imidazolidin-1- | |
| | yl)benzonitrile | |
| 54-1 | (R)-3-(1-(4-cyanophenyl)-5,5-dimethyl-2-oxo-3-(p- | C |
| | tolyl)imidazolidin-4-yl)benzonitrile | |
| 54-2 | (S)-3-(1-(4-cyanophenyl)-5,5-dimethyl-2-oxo-3-(p- | A |
| | tolyl)imidazolidin-4-yl)benzonitrile | |
| 51-5 | 4,4′-((4S,5S)-4-ethyl-4-methyl-2-oxo-5-(3- | B |
| | (trifluoromethoxy)phenyl)imidazolidine-1,3- | |
| | diyl)dibenzonitrile | |
| 52-5 | 4,4′-((4S,5S)-4-methyl-2-oxo-4-propyl-5-(3- | A |
| | (trifluoromethoxy)phenyl)imidazolidine-1,3- | |
| | diyl)dibenzonitrile | |
| 55-1 | (R)-3-(7-(4-cyanophenyl)-6-oxo-5-(p-tolyl)-2-oxa- | C |
| | 5,7-diazaspiro[3.4]octan-8-yl)benzonitrile | |
| 55-2 | (S)-3-(7-(4-cyanophenyl)-6-oxo-5-(p-tolyl)-2-oxa- | B |
| | 5,7-diazaspiro[3.4]octan-8-yl)benzonitrile | |
| 56-1 | (R)-1-(4-chlorophenyl)-4,4-dimethyl-3-(p-tolyl)-5- | C |
| | (3-(trifluoromethoxy)phenyl)imidazolidin-2-one | |
| 56-2 | (S)-1-(4-chlorophenyl)-4,4-dimethyl-3-(p-tolyl)-5- | A |
| | (3-(trifluoromethoxy)phenyl)imidazolidin-2-one | |
| 57-1 | (R)-3-(5-(4-cyanophenyl)-6-oxo-7-(p-tolyl)-2-oxa- | C |
| | 5,7-diazaspiro[3.4]octan-8-yl)benzonitrile | |
| 57-2 | (S)-3-(5-(4-cyanophenyl)-6-oxo-7-(p-tolyl)-2-oxa- | A |
| | 5,7-diazaspiro[3.4]octan-8-yl)benzonitrile | |
| 2 | 1,3-bis(4-bromophenyl)-4-methyl-5- | NT |
| | phenylimidazolidin-2-one | |
| 4 | 1,3-bis(4-chlorophenyl)-4-methyl-5-(3- | NT |
| | (trifluoromethoxy)phenyl)imidazolidin-2-one | |
| 5 | 3-(1,3-bis(4-chlorophenyl)-5-methyl-2- | NT |
| | oxoimidazolidin-4-yl)benzonitrile | |
| 11 | 1,3-bis(4-bromophenyl)-4-methyl-5-(3- | NT |
| | (trifluoromethoxy)phenyl)imidazolidin-2-one | |
| 13 | 1,3-bis(4-chlorophenyl)-3a-(3- | NT |
| | (trifluoromethyl)phenyl) | |
| | hexahydrocyclopenta[d]imidazol-2(1H)-one | |
| 15 | 1,3-bis(4-chlorophenyl)-4-(3- | NT |
| | (trifluoromethoxy)phenyl)-1,3-diazaspiro[4.4]nonan- | |
| | 2-one | |
| 16 | 1,3-bis(4-chlorophenyl)-3a-(3-(difluoromethyl)phenyl) | NT |
| | hexahydrocyclopenta[d]imidazol-2(1H)-one | |
| 19 | 1,3-bis(4-chlorophenyl)-4-ethyl-4-methyl-5-(3- | NT |
| | (trifluoromethoxy)phenyl)imidazolidin-2-one | |
| 20 | 4,4-dimethyl-5-(3-(trifluoromethoxy)phenyl)-1,3- | NT |
| | bis(4-(trifluoromethyl)phenyl)imidazolidin-2-one | |
| 21 | 4,4-dimethyl-1,3-di-p-tolyl-5-(3- | NT |
| | (trifluoromethoxy)phenyl)imidazolidin-2-one | |
| 22-1 | (4R)-1,3-bis(4-chlorophenyl)-4-methyl-4-propyl-5-(3- | NT |
| | (trifluoromethoxy)phenyl)imidazolidin-2-one | |
| 22-4 | (4S)-1,3-bis(4-chlorophenyl)-4-methyl-4-propyl-5-(3- | NT |
| | (trifluoromethoxy)phenyl)imidazolidin-2-one | |
| 23 | 1,3-bis(4-chlorophenyl)-3a-(3- | NT |
| | (difluoromethoxy)phenyl) | |
| | hexahydrocyclopenta[d]imidazol-2(1H)-one | |
| 24 | 3-(5,5-dimethyl-2-oxo-1,3-bis(4-(trifluoromethyl) | NT |
| | phenyl)imidazolidin-4-yl)benzonitrile | |
| 26 | 4,4-dimethyl-1,3-diphenyl-5-(3- | NT |
| | (trifluoromethoxy)phenyl)imidazolidin-2-one | |
| 28 | 1,3-bis(3-chlorophenyl)-4,4-dimethyl-5-(3- | NT |
| | (trifluoromethoxy)phenyl)imidazolidin-2-one | |
| 29 | 5,7-bis(4-chlorophenyl)-8-(3- | NT |
| | (trifluoromethoxy)phenyl)-2-oxa-5,7- | |
| | diazaspiro[3.4]octan-6-one | |
| 30 | 3-(5,7-bis(4-chlorophenyl)-6-oxo-2-oxa-5,7- | NT |
| | diazaspiro[3.4]octan-8-yl)benzonitrile | |
| 31 | 1,3-bis(4-chlorophenyl)-3a-(3- | NT |
| | (trifluoromethoxy)phenyl)hexahydropyrano[3,4- | |
| | d]imidazol-2(3H)-one | |
| 32 | 3-(6-oxo-5,7-di-p-tolyl-2-oxa-5,7- | NT |
| | diazaspiro[3.4]octan-8-yl)benzonitrile | |
| 33 | 3-(1,3-bis(4-chlorophenyl)-5-methyl-2-oxo-5- | NT |
| | propylimidazolidin-4-yl)benzonitrile | |
| 34 | 3-(1,3-bis(4-chlorophenyl)-5-ethyl-5-methyl-2- | NT |
| | oxoimidazolidin-4-yl)benzonitrile | |
| 35 | 1,3-di-p-tolyl-4-(3-(trifluoromethoxy)phenyl)-1,3- | NT |
| | diazaspiro[4.4]nonan-2-one | |
| 36 | 4,4′-(6-oxo-8-(3-(trifluoromethoxy)phenyl)-2-oxa- | NT |
| | 5,7-diazaspiro[3.4]octane-5,7-diyl)dibenzonitrile | |
| 37 | 1,3-bis(4-chlorophenyl)-4-(methoxymethyl)-4- | NT |
| | methyl-5-(3-(trifluoromethoxy)phenyl)imidazolidin- | |
| | 2-one | |
| 38 | 3-(1,3-bis(4-chlorophenyl)-5-(methoxymethyl)-5- | NT |
| | methyl-2-oxoimidazolidin-4-yl)benzonitrile | |
| 39 | 4,4′-(4-methyl-2-oxo-5-(3-(trifluoromethoxy)phenyl)- | NT |
| | 4-(trifluoromethyl)imidazolidine-1,3- | |
| | diyl)dibenzonitrile | |
| 40 | 4-(3-(4-chlorophenyl)-4,4-dimethyl-2-oxo-5-(3- | NT |
| | (trifluoromethoxy)phenyl)imidazolidin-1- | |
| | yl)benzonitrile | |
| 41 | 4-(3-(4-chlorophenyl)-5,5-dimethyl-2-oxo-4-(3- | NT |
| | (trifluoromethoxy)phenyl)imidazolidin-1- | |
| | yl)benzonitrile | |
| 42 | 4-(6-oxo-5-(p-tolyl)-8-(3-(trifluoromethoxy)phenyl)- | NT |
| | 2-oxa-5,7-diazaspiro[3.4]octan-7-yl)benzonitrile | |
| 43 | 4-(6-oxo-7-(p-tolyl)-8-(3-(trifluoromethoxy)phenyl)- | NT |
| | 2-oxa-5,7-diazaspiro[3.4]octan-5-yl)benzonitrile | |
| 44 | 4-(3-(4-bromophenyl)-5-methyl-2-oxo-4-(3- | NT |
| | (trifluoromethoxy)phenyl)-5-(trifluoromethyl) | |
| | imidazolidin-l-yl)benzonitrile | |
| 45 | 3-(5,7-bis(4-chlorophenyl)-2,2-difluoro-6-oxo-5,7- | NT |
| | diazaspiro[3.4]octan-8-yl)benzonitrile | |
| 46 | 4-(7-(4-chlorophenyl)-6-oxo-8-(3- | NT |
| | (trifluoromethoxy)phenyl)-2-oxa-5,7- | |
| | diazaspiro[3.4]octan-5-yl)benzonitrile | |
| 47 | 4-(5-(4-chlorophenyl)-6-oxo-8-(3- | NT |
| | (trifluoromethoxy)phenyl)-2-oxa-5,7- | |
| | diazaspiro[3.4]octan-7-yl)benzonitrile | |
| 48 | 3-(3-(4-cyanophenyl)-5,5-dimethyl-2-oxo-1-(p- | NT |
| | tolyl)imidazolidin-4-yl)benzonitrile | |
| 49 | 7-(4-chlorophenyl)-5-(p-tolyl)-8-(3- | NT |
| | (trifluoromethoxy)phenyl)-2-oxa-5,7- | |
| | diazaspiro[3.4]octan-6-one | |
| 50 | 4-(4,4-dimethyl-2-oxo-3-(p-tolyl)-5-(3- | NT |
| | (trifluoromethoxy)phenyl)imidazolidin-1- | |
| | yl)benzonitrile | |
| 51-1 | 4,4′-((4R)-4-ethyl-4-methyl-2-oxo-5-(3- | NT |
| | (trifluoromethoxy)phenyl)imidazolidine-1,3- | |
| | diyl)dibenzonitrile | |
| 51-4 | 4,4′-((4S)-4-ethyl-4-methyl-2-oxo-5-(3- | NT |
| | (trifluoromethoxy)phenyl)imidazolidine-1,3- | |
| | diyl)dibenzonitrile | |
| 52-1 | 4,4′-((4R)-4-methyl-2-oxo-4-propyl-5-(3- | NT |
| | (trifluoromethoxy)phenyl)imidazolidine-1,3- | |
| | diyl)dibenzonitrile | |
| 52-4 | 4,4′-((4S)-4-methyl-2-oxo-4-propyl-5-(3- | NT |
| | (trifluoromethoxy)phenyl)imidazolidine-1,3- | |
| | diyl)dibenzonitrile | |
| 53 | 4-(5,5-dimethyl-2-oxo-3-(p-tolyl)-4-(3- | NT |
| | (trifluoromethoxy)phenyl)imidazolidin-1- | |
| | yl)benzonitrile | |
| 54 | 3-(1-(4-cyanophenyl)-5,5-dimethyl-2-oxo-3-(p- | NT |
| | tolyl)imidazolidin-4-yl)benzonitrile | |
| 55 | 3-(7-(4-cyanophenyl)-6-oxo-5-(p-tolyl)-2-oxa-5,7- | NT |
| | diazaspiro[3.4]octan-8-yl)benzonitrile | |
| 56 | 1-(4-chlorophenyl)-4,4-dimethyl-3-(p-tolyl)-5-(3- | NT |
| | (trifluoromethoxy)phenyl)imidazolidin-2-one | |
| 57 | 3-(5-(4-cyanophenyl)-6-oxo-7-(p-tolyl)-2-oxa-5,7- | NT |
| | diazaspiro[3.4]octan-8-yl)benzonitrile | |

### Twitcher Mouse Model

The twitcher is a naturally-occurring mouse mutant caused by an abnormality in the gene coded for galactosylceramidase (Kobayashi et al., Brain Res. 202: 479-483 (1980); Suzuki and Suzuki, Am. J Path. 111: 394-397 (1983)). It is therefore genetically equivalent to human globoid cell leukodystrophy (Krabbe disease). Affected mice develop clinical symptoms at the onset of the active myelination period and, if untreated, die by 35± days. The pathology is very similar to that in human disease. Toxicity of galactosylsphingosine (psychosine) that accumulates abnormally in the nervous system is considered to be primarily responsible for the pathogenesis.

To evaluate the potential efficacy of different compounds described herein in Krabbe disease, the Twitcher mouse model was used. This model is as described in Hawkins-Salsbury et al., J. Neurosci. 35(16): 6495-6505 (2015), which is incorporated herein by reference in its entirety.

If tested compounds are found which have a marked effect on the experimental endpoints (e.g., life span with improved motor function, GALC expression, psychosine levels, and neuroinflammation), an additional experiment is performed looking at effects on activity, inverted screen, and bar crossing tests, as well as average survival time, compared to vehicle-treated (control) mice.

Other objects, features and advantages of the compounds, methods and compositions described herein will become apparent from the following description. It should be understood, however, that the description and the specific examples, while indicating specific embodiments, are given by way of illustration only, since various changes and modifications within the spirit and scope of the present description will become apparent from this detailed description.

All publications including patents, patent applications and published patent applications cited herein are hereby incorporated by reference for all purposes.

We claim:

1. A compound of Formula I: 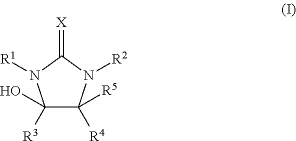
wherein:
X is O or S;
R¹ is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with 1 or 2 groups selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl; wherein the spirocycloalkyl and spiroheterocycloalkyl groups are each optionally substituted with 1 or 2 groups selected from halo, cyano, hydroxy, C₁₋₃ alkyl, alkoxy, amino, alkylamino, and dialkylamino;
or a single stereoisomer, mixture of stereoisomers, or pharmaceutically acceptable salt thereof.

2. The compound or pharmaceutically acceptable salt thereof of claim 1, wherein X is O.

3. The compound or pharmaceutically acceptable salt thereof of claim 1, wherein X is S.

4. The compound or pharmaceutically acceptable salt thereof of any one of claims 1-3, wherein R¹ is optionally substituted phenyl.

5. The compound or pharmaceutically acceptable salt thereof of any one of claims 1-4, wherein R¹ is optionally substituted with 1 or 2 groups selected from halo, cyano, alkyl, haloalkyl, alkoxy, cycloalkyloxy, amino, alkylamino, and dialkylamino.

6. The compound or pharmaceutically acceptable salt thereof of any one of claims 1-5, wherein R¹ is phenyl substituted with 1 or 2 groups selected from halo, cyano, alkyl, haloalkyl, alkoxy, and cycloalkyloxy.

7. The compound or pharmaceutically acceptable salt thereof of any one of claims 1-6, wherein R¹ is phenyl substituted with 1 or 2 groups selected from halo and alkyl.

8. The compound or pharmaceutically acceptable salt thereof of any one of claims 1-7, wherein R¹ is phenyl substituted with 1 or 2 groups selected from chloro, bromo, methyl, and ethyl.

9. The compound or pharmaceutically acceptable salt thereof of any one of claims 1-8, wherein R¹ and R² are the same.

10. The compound or pharmaceutically acceptable salt thereof of any one of claims 1-9, wherein R³ is alkyl, haloalkyl, hydroxyalkyl, alkoxycarbonyl, heterocycloalkyl, aryl, or heteroaryl; wherein the heterocycloalkyl, aryl, and heteroaryl groups are each optionally substituted.

11. The compound or pharmaceutically acceptable salt thereof of any one of claims 1-10, wherein R³ is aryl or heteroaryl; wherein the aryl or heteroaryl groups are each optionally substituted.

12. The compound or pharmaceutically acceptable salt thereof of any one of claims 1-11, wherein R³ is optionally substituted phenyl.

13. The compound or pharmaceutically acceptable salt thereof of any one of claims 1-12, wherein R³ is substituted phenyl.

14. The compound or pharmaceutically acceptable salt thereof of any one of claims 1-11, wherein R³ is optionally substituted heteroaryl.

15. The compound or pharmaceutically acceptable salt thereof of any one of claims 1-11 and 14, wherein R³ is pyrrolidinyl, furanyl, thienyl, thiazolyl, pyridinyl, benzimidazolyl, benzofuranyl, benzothienyl, or benzothiazolyl; wherein each are optionally substituted.

16. The compound or pharmaceutically acceptable salt thereof of any one of claims 1-15, wherein R³ is optionally substituted with 1 or 2 groups selected from halo, cyano, hydroxy, alkyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo.

17. The compound or pharmaceutically acceptable salt thereof of any one of claims 1-16, wherein R³ is optionally substituted with 1 or 2 groups selected from halo, cyano, alkyl, alkynyl, alkoxy, and haloalkoxy.

18. The compound or pharmaceutically acceptable salt thereof of any one of claims 1-17, wherein R³ is optionally substituted with 1 or 2 groups selected from chloro, cyano, methyl, ethyl, methoxy, difluoromethoxy, and trifluoromethoxy.

19. The compound or pharmaceutically acceptable salt thereof of any one of claims 1-9, wherein R³ is hydrogen.

20. The compound or pharmaceutically acceptable salt thereof of any one of claims 1-19, wherein:
R⁴ is hydrogen, alkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, or cycloalkyl.

21. The compound or pharmaceutically acceptable salt thereof of any one of claims 1-20, wherein R⁴ is hydrogen.

22. The compound or pharmaceutically acceptable salt thereof of any one of claims 1-21, wherein R⁵ is hydrogen.

23. The compound or pharmaceutically acceptable salt thereof of any one of claims 1-19, wherein R⁴ and R⁵ and the carbon to which they are attached, combine to form carbonyl, spirocycloalkyl, or spiroheterocycloalkyl.

24. The compound or pharmaceutically acceptable salt thereof of claim 23, wherein R⁴ and R⁵ and the carbon to which they are attached, combine to form carbonyl.

25. The compound or pharmaceutically acceptable salt thereof of claim 23, wherein R⁴ and R⁵ and the carbon to which they are attached, combine to form spirocycloalkyl.

26. The compound or pharmaceutically acceptable salt thereof of claim 25, wherein R⁴ and R⁵ and the carbon to which they are attached, combine to form spirocyclopropyl.

27. The compound or pharmaceutically acceptable salt thereof of any one of claims 1-19, wherein
R⁴ is hydrogen, alkyl, cycloalkyl, heterocycloalkyl, phenyl, or thienyl; wherein each is optionally substituted; and
R⁵ is hydrogen, alkyl, alkoxy, or cycloalkyl.

28. The compound or pharmaceutically acceptable salt thereof of claim 27, wherein R⁴ is cycloalkyl, heterocycloalkyl, phenyl, or thienyl.

29. The compound or pharmaceutically acceptable salt thereof of claim 27, wherein R⁴ is alkyl, cycloalkyl, or phenyl.

30. The compound or pharmaceutically acceptable salt thereof of claim 27, wherein R⁴ is hydrogen or alkyl.

31. The compound or pharmaceutically acceptable salt thereof of any one of claims 27, 29, and 30, wherein R⁴ is alkyl.

32. The compound or pharmaceutically acceptable salt thereof of claim 29, wherein R⁴ is methyl, ethyl, propyl, or cyclopropyl.

33. The compound or pharmaceutically acceptable salt thereof of claim 29, wherein R⁴ is methyl.

34. The compound or pharmaceutically acceptable salt thereof of any one of claims 27-33, wherein R⁵ is hydrogen or alkyl.

35. The compound or pharmaceutically acceptable salt thereof of any one of claims 27-33, wherein R⁵ is alkyl or cycloalkyl.

36. A compound of Formula (I): 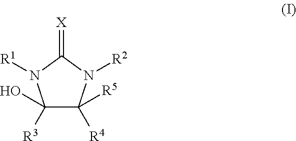
wherein:
X is O or S;
R¹ is aryl optionally substituted with 1 or 2 groups selected from halo, cyano, alkyl, haloalkyl, alkoxy, cycloalkyloxy, amino, alkylamino, and dialkylamino;
R² is aryl optionally substituted with 1 or 2 groups selected from halo, cyano, alkyl, haloalkyl, alkoxy, cycloalkyloxy, amino, alkylamino, and dialkylamino;
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, hydroxy, alkyl, alkenyl, alkynyl, haloalkyl, hydroxyalkyl, alkoxy, haloalkoxy, cycloalkyloxy, (cycloalkyl)alkoxy, dialkylamino, alkylcarbonyl, cycloalkyl, heterocycloalkyl, and phenyl optionally substituted with one group selected from halo;
R⁴ is hydrogen, alkyl, cycloalkyl, heterocycloalkyl, phenyl, or thienyl; wherein the alkyl is optionally substituted with 1 or 2 groups selected from halo, alkoxy, amino, alkylamino, dialkylamino; and
R⁵ is hydrogen, alkyl, or cycloalkyl;
or R⁴ and R⁵ and the carbon to which they are attached combine to form carbonyl, spirocycloalkyl, or spiroheterocycloalkyl optionally substituted with halo;
or a single stereoisomer, mixture of stereoisomers, or pharmaceutically acceptable salt thereof.

37. A compound of Formula Ia: 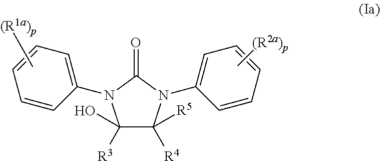
wherein:
R³ is hydrogen, alkyl, haloalkyl, hydroxyalkyl, alkoxycarbonyl, heterocycloalkyl, aryl, or heteroaryl; wherein the heterocycloalkyl, aryl, and heteroaryl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
R⁴ is hydrogen, alkyl, alkoxyalkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl;
R^{1a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R^{2a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
p is 0, 1, or 2; and
q is 0, 1, or 2;
or a single stereoisomer, mixture of stereoisomers, or pharmaceutically acceptable salt thereof.

38. A compound of Formula Ib: 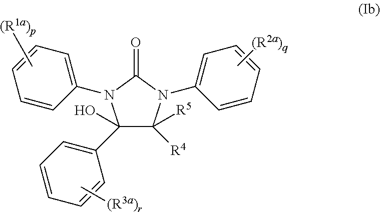
wherein:
R⁴ is hydrogen, alkyl, cycloalkyl, heterocycloalkyl, phenyl, or thienyl; wherein the alkyl is optionally substituted with 1 or 2 groups selected from halo, cyano, alkoxy, amino, alkylamino, dialkylamino, cycloalkyl, and heterocycloalkyl; and wherein the cycloalkyl, heterocycloalkyl, phenyl, and thienyl groups are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
R⁵ is hydrogen, alkyl, alkoxy, or cycloalkyl;
or R⁴ and R⁵ and the carbon to which they are attached combine to form carbonyl, spirocycloalkyl, or spiroheterocycloalkyl;
R^{1a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R^{2a} is selected from halo, cyano, nitro, alkyl, haloalkyl, hydroxyalkyl, alkoxy, cycloalkyloxy, amino, alkylamino, dialkylamino, alkylcarbonyl, alkoxycarbonyl, aminocarbonyl, alkylaminocarbonyl, dialkylaminocarbonyl, and alkylcarbonylamino;
R^{3a} selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, heterocycloalkyl, and phenyl optionally substituted with one group selected from halo, alkyl, and haloalkyl;
p is 0, 1, or 2;
q is 0, 1, or 2; and
r is 0, 1, or 2;
or a single stereoisomer, mixture of stereoisomers, or pharmaceutically acceptable salt thereof.

39. A compound selected from the group consisting of the compounds of Examples 1-2, 4-17, 19-21, 23-26, 28-36, 38-39, 42-43, 45, 47, 49, 51-64, 66-69, and 71-141, 143-242, or a single stereoisomer, mixture of stereoisomers, or pharmaceutically acceptable salt thereof.

40. A compound of Formula II: 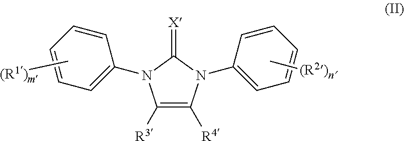
wherein:
X′ is O or S;
R^{1′} is halo, cyano, alkyl, haloalkyl, or alkoxy;
R^{2′} is halo, cyano, alkyl, haloalkyl, or alkoxy;
R^{3′} is alkyl, phenyl, heteroaryl with 5-6 ring atoms, or phenylcarbonyl, wherein the phenyl, heteroaryl, or phenylcarbonyl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl;
R^{4′} is hydrogen, alkyl, alkoxyalkyl, C₃₋₅ cycloalkyl, or 3-6 membered heterocycloalkyl, wherein the cycloalkyl and heterocycloalkyl groups are each independently optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R^{3′} and R^{4′} and the carbons to which they are attached combine to form a 5-6 membered cycloalkylene;
m′ is 1 or 2;
n′ is 1 or 2;
with the proviso that when R^{1′} and R^{2′} are each methoxy, then R^{3′} cannot be methoxy-substituted phenyl;
or a pharmaceutically acceptable salt thereof.

41. The compound or pharmaceutically acceptable salt thereof of claim 40, wherein X′ is O.

42. The compound or pharmaceutically acceptable salt thereof of claim 40, wherein X′ is S.

43. The compound or pharmaceutically acceptable salt thereof of any one of claims 40-42, wherein R^{1′} and R^{2′} are each independently selected from halo.

44. The compound or pharmaceutically acceptable salt thereof of claim 43, wherein R^{1′} and R^{2′} are each independently selected from chloro and bromo.

45. The compound or pharmaceutically acceptable salt thereof of claim 44, wherein R^{1′} and R^{2′} are each chloro.

46. The compound or pharmaceutically acceptable salt thereof of claim 44, wherein R^{1′} and R^{2′} are each bromo.

47. The compound or pharmaceutically acceptable salt thereof of any one of claims 40-42, wherein R^{1′} and R^{2′} are each independently selected from halo and alkyl.

48. The compound or pharmaceutically acceptable salt thereof of claim 47, wherein R^{1′} and R^{2′} are each independently selected from halo and methyl.

49. The compound or pharmaceutically acceptable salt thereof of any one of claims 40-42, wherein R^{1′} and R^{2′} are each independently selected from halo, cyano, haloalkyl, and alkoxy.

50. The compound or pharmaceutically acceptable salt thereof of claim 49, wherein R^{1′} and R^{2′} are each independently selected from halo, cyano, and haloalkyl.

51. The compound or pharmaceutically acceptable salt thereof of claim 50, wherein R^{1′} and R^{2′} are each independently selected from halo and cyano.

52. The compound or pharmaceutically acceptable salt thereof of any one of claim 40-51, wherein R^{1′} and R^{2′} are at the para position.

53. The compound or pharmaceutically acceptable salt thereof of any one of claims 40-52, wherein R^{3′} is alkyl, optionally substituted phenyl, or optionally substituted heteroaryl with 5-6 ring atoms.

54. The compound or pharmaceutically acceptable salt thereof of claim 53, wherein R^{3′} is optionally substituted phenyl, or optionally substituted heteroaryl with 5-6 ring atoms.

55. The compound or pharmaceutically acceptable salt thereof of claim 54, wherein R^{3′} is optionally substituted phenyl.

56. The compound or pharmaceutically acceptable salt thereof of claim 55, wherein R^{3′} is phenyl substituted with halo, cyano, alkyl, haloalkyl, hydroxyalkyl, haloalkoxy, or alkylcarbonyl.

57. The compound or pharmaceutically acceptable salt thereof of claim 54, wherein R^{3′} is optionally substituted heteroaryl with 5-6 ring atoms.

58. The compound or pharmaceutically acceptable salt thereof of any one of claims 40-57, wherein R^{4′} is hydrogen.

59. The compound or pharmaceutically acceptable salt thereof of any one of claims 40-57, wherein R^{4′} is alkyl, alkoxyalkyl, C₃₋₅ cycloalkyl, or 3-6 membered heterocycloalkyl.

60. The compound or pharmaceutically acceptable salt thereof of any one of claims 40-57, wherein R^{4′} is hydrogen, alkyl, or C₃₋₅ cycloalkyl.

61. The compound or pharmaceutically acceptable salt thereof of any one of claims 59 and 60, wherein R^{4′} is alkyl or C₃₋₅ cycloalkyl.

62. The compound or pharmaceutically acceptable salt thereof of claim 40, wherein:
X′ is O;
R^{1′} and R^{2′} are each independently selected from halo;
R^{3′} is alkyl, phenyl, heteroaryl with 5 ring atoms, or phenylcarbonyl, wherein the phenyl, heteroaryl, or phenylcarbonyl are each optionally substituted with a group selected from halo, cyano, alkyl, haloalkyl, hydroxyalkyl, haloalkoxy, and alkylcarbonyl;
R^{4′} is hydrogen, alkyl, alkoxyalkyl, C₃₋₅ cycloalkyl, or 3-6 membered heterocycloalkyl;
or R^{3′} and R^{4′} and the carbons to which they are attached combine to form a 5-6 membered cycloalkylene; and
m′ and n′ are each 1.

63. A compound of Formula IIa: 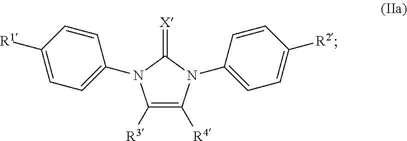
X′ is O or S;
R^{1′} is halo, cyano, alkyl, haloalkyl, or alkoxy;
R^{2′} is halo, cyano, alkyl, haloalkyl, or alkoxy;
R^{3′} is alkyl, phenyl, heteroaryl with 5-6 ring atoms, or phenylcarbonyl, wherein the phenyl, heteroaryl, or phenylcarbonyl are each optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl;
R^{4′} is hydrogen, alkyl, alkoxyalkyl, C₃₋₅ cycloalkyl, or 3-6 membered heterocycloalkyl, wherein the cycloalkyl and heterocycloalkyl groups are each independently optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl; and
or R^{3′} and R^{4′} and the carbons to which they are attached combine to form a 5-6 membered cycloalkylene;
or a pharmaceutically acceptable salt thereof.

64. A compound of Formula IIb: 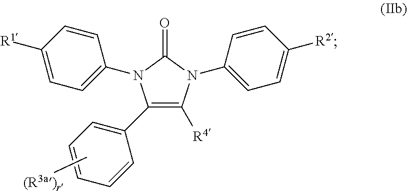
R^{1′} is halo, cyano, alkyl, haloalkyl, or alkoxy;
R^{2′} is halo, cyano, alkyl, haloalkyl, or alkoxy;
R^{4′} is hydrogen, alkyl, alkoxyalkyl, C₃₋₅ cycloalkyl, or 3-6 membered heterocycloalkyl, wherein the cycloalkyl and heterocycloalkyl groups are each independently optionally substituted with 1 or 2 groups selected from halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
R^{3a′} is selected from halo, cyano, nitro, hydroxy, alkyl, alkenyl, alkynyl, haloalkyl, hydroxyalkyl, alkoxy, hydroxyalkoxy, haloalkoxy, cycloalkyloxy, (cycloalkyl)alkoxy, amino, alkylamino, dialkylamino, alkylcarbonyl, alkoxycarbonyl, aminocarbonyl, alkylaminocarbonyl, dialkylaminocarbonyl, cycloalkyl, and heterocycloalkyl; and
r′ is 0, 1, or 2;
or a pharmaceutically acceptable salt thereof.

65. A compound selected from the group of Table 1 consisting of the Examples 1-12, 13, 13-1, 13-2, 14-24, 25, 25-1, 25-2, 26-28, 29-1, 29-2, 30-35, 36, 36-1, 36-2, 37-41, 42, 42-1, 42-2, 43, 44, 45, 45-1, 45-2, 46-48, 49, 49-1, 49-2, 50, 51, 51-1, 51-2, 52, 53-1, 53-2, 54-61, 62, 62-1, 62-2, 63-65, 66, 66-1, 66-2, 67, 67-1, 67-2, 68, 68-1, 68-2, 69, 69-1, 69-2, 70, 71-1, 71-2, 72, 73, 73-1, 73-2, 74-1, 74-2, 75-1, 75-2, 76-1, 76-2, 77-1, 77-2, 78, 79, 80-1, 80-2, 81, 81-1, 81-2, 82, 82-1, 82-2, 83, 83-1, 83-2, 84-1, 84-2, 85, 86-1, 86-2, 87, 87-1, 87-2, 88, 88-1, 88-2, 89, 89-1, 89-2, 90, 90-1, 90-2, 91-1, 91-2, 92-1, 92-2, 93, 93-1, 93-2, 94, 94-1, 94-2, 95, 96, 96-1, 96-2, 97, 97-1, 97-2, 98, 99, 99-1, 99-2, 100-1, 100-2, 101, 101-1, 101-2, 102, 103, 103-1, 103-2, 104, 104-1, 104-2, 105, 105-1, 105-2, 106, 106-1, 106-2, 107, 108, 109, 110, 111, 112-1, 112-2, 113, 114, 114-1, 114-2, 115, 115-1, 115-2, 116, 116-1, 116-2, 117-1, 117-2, 118, 118-1, 118-2, 119, 119-1, 119-2, 120, 121, 121-1, 121-2, 121-3, 121-4, 122, 123-1, 123-2, 124, 125, 126, 126-1, 126-2, 127, 128, 129, 129-1, 129-2, 130, 130-1, 130-2, 131, 132, 132-1, 132-2, 133-135, 136-1, 136-2, 137, 138, 139, 139-1, 139-2, 140, 140-1, 140-2, 141, 143-148, 149, 149-1, 149-2, 150-158, 159, 159-1, 159-2, 160-162, 161, 163-1, 163-2, 164, 165, 166, 166-1, 166-2, 167-169, 170, 170-1, 170-2, 171-176, 177, 177-1, 177-2, 178, 179, 180, 180-1, 180-2, 181-184, 185-1, 185-2, 186, 187-1, 187-2, 188, 189, 190, 190-1, 190-2, 191, 191-1, 191-2, 192, 193, 194, 194-1, 194-2, 195, 196, 196-1, 196-2, 197-208, 209-1, 209-2, 210-217, 218, 218-1, 218-2, 219-1, 219-2, 220, 221, 221-2, 221-1, 222, 222-1, 222-2, 223, 223-1, 223-2, 224-229, 230, 230-1, 230-2, 231, 232, 233, 233-1, 233-2, 234, 234-1, 234-2, 234-3, 234-4, 235, 235-1, 235-2, 236, 237, 238-1, 238-2, and 239-267, or a pharmaceutically acceptable salt thereof.

66. A compound selected from the group of Table 1 consisting of the Examples 268-295, 296, 296-1, 296-2, 296-3, 296-4, 297, 297-1, 297-2, 298-318, 319-1, 319-2, 320-1, 320-2, 321-323, 324-1, 324-2, 325-328, 329, 329-1, 329-2, 330, 331, 331-1, 331-2, and 332-338, or a pharmaceutically acceptable salt thereof.

67. A compound selected from the group of Table 1 consisting of the Examples 3, 18, 22, 27, 37, 40, 41, 44, 46, 48, 50, 65, 70, and 243-267, or a pharmaceutically acceptable salt thereof.

68. A pharmaceutical composition comprising the compound of any one of claims 1-65, optionally as a single stereoisomer, or mixture of stereoisomers, and additionally optionally as a pharmaceutically acceptable salt thereof, and a pharmaceutically acceptable excipient.

69. A method of treating a disease or disorder comprising administering to a subject having the disease or disorder the compound of any one of claims 1-65 or the pharmaceutical composition of claim 68.

70. The method of claim 69, wherein the disease or disorder is a lysosomal storage disease.

71. The method of claim 70, wherein the lysosomal storage disease is Krabbe disease or Metachromatic Leukodystrophy.

72. A compound of Formula VII: 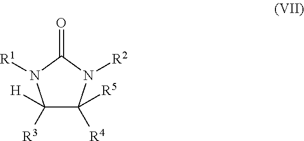
wherein:
R¹ is aryl optionally substituted with a group selected from the group consisting of halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R² is aryl optionally substituted with a group selected from the group consisting of halo, cyano, nitro, alkyl, alkenyl, haloalkoxy, haloalkyl, hydroxyalkyl, alkoxy, amino, alkylamino, dialkylamino, alkylcarbonyl, alkoxycarbonyl, aminocarbonyl, alkylaminocarbonyl, dialkylaminocarbonyl, and alkylcarbonylamino;
R³ is aryl optionally substituted with a group selected from the group consisting of halo, cyano, nitro, hydroxy, alkyl, alkenyl, alkynyl, haloalkyl, hydroxyalkyl, alkoxy, hydroxyalkoxy, haloalkoxy, amino, alkylamino, dialkylamino, alkylcarbonyl, alkoxycarbonyl, aminocarbonyl, alkylaminocarbonyl, and dialkylaminocarbonyl;
or R³ and R⁴, together with the carbon atoms to which they are attached combine to form a C₅₋₆ cycloalkyl or a 5-6 membered heterocycloalkyl;
R⁴ is hydrogen, alkyl, alkenyl, haloalkyl, or alkoxyalkylene;
R⁵ is hydrogen, alkyl, alkenyl, haloalkyl, alkoxyalkylene, cycloalkyl, or aryl, wherein the aryl is optionally substituted with a group selected from the group consisting of alkyl, cyano, haloalkyl, hydroxy, alkoxy, and haloalkoxy;
or R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 1 or 2 groups selected from halo, cyano, hydroxy, C₁₋₃ alkyl, alkoxy, amino, alkylamino, and dialkylamino; and
provided that R⁴ and R⁵ are not both hydrogen; and
provided that the compound is not (4S,5S)-4-(tert-butyl)-1,3-bis(4-methoxyphenyl)-5-phenylimidazolidin-2-one; and
optionally a single stereoisomer or mixture of stereoisomers thereof and additionally optionally a pharmaceutically acceptable salt thereof.

73. The compound or pharmaceutically acceptable salt thereof of claim 72, wherein R¹ and R², each independently, are optionally substituted phenyl.

74. The compound or pharmaceutically acceptable salt thereof of claim 72 or 73, wherein R¹ and R², each independently, are optionally substituted with a group selected from the group consisting of alkyl, cyano, halo, and haloalkyl.

75. The compound or pharmaceutically acceptable salt thereof of any one of claims 72-74, wherein R¹ and R², each independently, are phenyl substituted with a group selected from the group consisting of alkyl, cyano, halo, and haloalkyl.

76. The compound or pharmaceutically acceptable salt thereof of any one of claims 72-75, wherein R¹ and R², each independently, are phenyl substituted with a group selected from the group consisting of fluoro, chloro, bromo, iodo, methyl, ethyl, propyl, trifluoromethyl, and cyano.

77. The compound or pharmaceutically acceptable salt thereof of any one of claims 72-75, wherein R¹ and R², each independently, are phenyl substituted with an alkyl group.

78. The compound or pharmaceutically acceptable salt thereof of any one of claims 72-75, wherein R¹ and R², each independently, are phenyl substituted with a halo group.

79. The compound or pharmaceutically acceptable salt thereof of claim 72 or 73, wherein R¹ and R², each independently, are an unsubstituted phenyl.

80. The compound or pharmaceutically acceptable salt thereof of any one of claims 72-78, wherein R¹ and R² are the same.

81. The compound or pharmaceutically acceptable salt thereof of any one of claims 72-78, wherein R¹ and R² are different.

82. The compound or pharmaceutically acceptable salt thereof of any one of claims 72-76, 78, or 80, wherein R¹ and R², each independently, are phenyl substituted with chloro.

83. The compound or pharmaceutically acceptable salt thereof of any one of claims 72-82, wherein R³ is optionally substituted phenyl.

84. The compound or pharmaceutically acceptable salt thereof of any one of claims 72-83, wherein R³ is optionally substituted with a group selected from alkyl, cyano, halo, haloalkyl, and haloalkoxy.

85. The compound or pharmaceutically acceptable salt thereof of any one of claims 72-84, wherein R³ is phenyl substituted with a group selected from alkyl, cyano, halo, haloalkyl, and haloalkoxy.

86. The compound or pharmaceutically acceptable salt thereof of any one of claims 72-85, wherein R³ is phenyl substituted with a group selected from fluoro, chloro, bromo, iodo, methyl, ethyl, propyl, trifluoromethyl, difluoromethyl, cyano, trifluoromethoxy, and difluoromethoxy.

87. The compound or pharmaceutically acceptable salt thereof of any one of claims 72-85, wherein R³ is phenyl substituted with an alkyl group.

88. The compound or pharmaceutically acceptable salt thereof of any one of claims 72-85, wherein R³ is phenyl substituted with a halo group.

89. The compound or salt of any one of claims 72-88, wherein R³ is substituted phenyl.

90. The compound or pharmaceutically acceptable salt thereof of any one of claims 72-82, wherein R³ and R⁴, together with the carbon atoms to which they are attached combine to form C₅₋₆ cycloalkyl or 5-6 membered heterocycloalkyl.

91. The compound or pharmaceutically acceptable salt thereof of any one of claims 72-82, or 90, wherein R³ and R⁴, together with the carbon atoms to which they are attached combine to form C₅₋₆ cycloalkyl.

92. The compound or pharmaceutically acceptable salt thereof of any one of claims 72-82, 90, or 91, wherein the cycloalkyl is cyclopentyl or cyclohexyl.

93. The compound or pharmaceutically acceptable salt thereof of any one of claims 72-82, or 90, wherein R³ and R⁴, together with the carbon atoms to which they are attached combine to form 5-6 membered heterocycloalkyl.

94. The compound or pharmaceutically acceptable salt thereof of any one of claims 72-82, or 90, wherein the heterocycloalkyl is

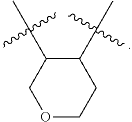 [SMILES: *C1CCOCC1*]

95. The compound or pharmaceutically acceptable salt thereof of any one of claims 90-94, wherein R⁵ is hydrogen.

96. The compound or pharmaceutically acceptable salt thereof of any one of claims 90-94, wherein R⁵ is phenyl substituted with a group selected from cyano, haloalkyl, hydroxy, and haloalkoxy.

97. The compound or pharmaceutically acceptable salt thereof of any one of claims 90-94, or 96, wherein R⁵ is phenyl substituted with a group selected from cyano, difluoromethyl, trifluoromethyl, hydroxy, trifluoromethoxy, and difluoromethoxy.

98. The compound or pharmaceutically acceptable salt thereof of any one of claims 72-89, wherein
R⁴ is hydrogen, alkyl, haloalkyl, or alkoxyalkylene; and
R⁵ is hydrogen, alkyl haloalkyl, or alkoxyalkylene.

99. The compound or pharmaceutically acceptable salt thereof of any one of claims 72-89, or 98 wherein R⁴ is alkyl, haloalkyl, or alkoxyalkylene, and R⁵ is hydrogen.

100. The compound or pharmaceutically acceptable salt thereof of any one of claims 72-89, 98, or 99, wherein R⁴ is methyl, ethyl, propyl, butyl, trifluoromethyl, or, methoxymethylene.

101. The compound or pharmaceutically acceptable salt thereof of any one of claims 72 to 89, or 98, wherein R⁵ is alkyl, haloalkyl, or alkoxyalkylene, and R⁴ is hydrogen, alkyl, haloalkyl, or alkoxyalkylene.

102. The compound or pharmaceutically acceptable salt thereof of any one of claims 72-89, 98, or 101, wherein R⁵ is methyl, ethyl, propyl, butyl, trifluoromethyl, or methoxymethylene.

103. The compound or pharmaceutically acceptable salt thereof of any one of claims 72-89, 98, 101 or 102, wherein R⁴ is hydrogen, methyl, ethyl, propyl, butyl, trifluoromethyl, or methoxymethylene.

104. The compound or pharmaceutically acceptable salt thereof of any one of claims 72-89, wherein R⁴ and R⁵, together with the carbon atom to which they are attached combine to form a C₃₋₆ spirocycloalkyl or a 3-6 membered spiroheterocycloalkyl, wherein the spirocycloalkyl is optionally substituted with 2 halo groups.

105. The compound or pharmaceutically acceptable salt thereof of any one of claims 72-89, or 104, wherein R⁴ and R⁵, together with the carbon atom to which they are attached combine to form a C₃₋₆ spirocycloalkyl, wherein the spirocycloalkyl is optionally substituted with 2 halo groups.

106. The compound or pharmaceutically acceptable salt thereof of any one of claims 72-89, 104, or 105, wherein the spirocycloalkyl is spirocyclopentyl or spirocyclobutyl, wherein the spirocyclobutyl is substituted with 2 fluoro groups.

107. The compound or pharmaceutically acceptable salt thereof of any one of claims 72-89 or 104, wherein R⁴ and R⁵, together with the carbon atom to which they are attached combine to form 3-6 membered spiroheterocycloalkyl.

108. The compound or pharmaceutically acceptable salt thereof of any one of claims 72-89, 104, or 107, wherein the spiroheterocycloalkyl is

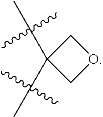 [SMILES: *C1(*)COC1]

109. A compound of Table 2; optionally as a single stereoisomer or mixture of stereoisomers thereof and additionally optionally a pharmaceutically acceptable salt thereof.

110. A compound selected from the group of Table 2 consisting of the compounds 1, 2-3, 2-1, 2-2, 3, 4-1, 4-2, 5-1, 5-2, 6, 7, 8, 9, 10, 8-1, 8-2, 10-1, 10-2, 7-1, 7-2, 6-1, 6-2, 9-1, 9-2, 11-1, 11-2, 12, 12-1, 12-2, 13-1, 13-2, 14, 15-1, 16-1, 16-2, 17, 18, 15-2, 15-1, 19-1, 19-2, 19-3, 19-4, 20-1, 20-2, 21-1, 21-2, 22-2, 22-3, 22-5, 23-1, 23-2, 24-1, 24-2, 25-1, 25-2, 17-1, 17-2, 26-1, 26-2, 14-1, 14-2, 27-1, 27-2, 28-1, 28-2, 29-1, 29-2, 30-1, 30-2, 31-1, 31-2, 32-1, 32-2, 33-3, 33-4, 34-1, 35-1, 35-2, 33-1, 33-2, 34-2, 36-1, 36-2, 37-1, 37-2, 34-3, 34-4, 38-1, 38-2, 39-1, 39-2, 40-1, 40-2, 41-1, 41-2, 42-1, 42-2, 43-1, 43-2, 44-1, 44-2, 45-1, 45-2, 46-1, 46-2, 47-1, 47-2, 48-1, 48-2, 49-1, 49-2, 50-1, 50-2, 51-2, 51-3, 51-6, 52-2, 52-3, 52-6, 53-1, 53-2, 54-1, 54-2, 51-5, 52-5, 55-1, 55-2, 56-1, 56-2, 57-1, and 57-2, or a single stereoisomer or mixture of stereoisomers thereof and additionally optionally as a pharmaceutically acceptable salt thereof.

111. A compound selected from the group of Table 2 consisting of the compounds 2-2, 6, 6-2, 7, 7-2, 8, 8-2, 9, 9-2, 10, 10-2, 11-2, 14, 15-2, 17, 17-1, 19-2, 19-3, 20-1, 21-2, 22-2, 22-3, 24-1, 25-1, 27-2, 28-2, 29-2, 30-2, 31-2, 32-2, 33-1, 33-4, 34-1, 34-3, 35-2, 36-2, 37-2, 38-2, 39-1, 40-2, 41-2, 42-1, 43-2, 44-2, 45-2, 46-1, 47-2, 48-2, 49-2, 50-1, 51-2, 52-2, 53-2, 54-2, 52-5, 56-2, and 57-2, or a single stereoisomer or mixture of stereoisomers thereof and additionally optionally as a pharmaceutically acceptable salt thereof.

112. A pharmaceutical composition comprising the compound of any one of claims 72-111, optionally as a single stereoisomer, or mixture of stereoisomers, and additionally optionally as a pharmaceutically acceptable salt thereof, and a pharmaceutically acceptable excipient.

113. A method of treating a disease or disorder comprising administering to a subject having the disease or disorder the compound or salt of any one of claims 72-111 or the pharmaceutical composition of claim 112.

114. The method of claim 113, wherein the disease or disorder is lysosomal storage disease.

115. The method of claim 114, wherein the lysosomal storage disease is Krabbe disease or Metachromatic Leukodystrophy.

116. The compound or pharmaceutically acceptable salt thereof of any one of claims 1-65 or the pharmaceutical composition of claim 68 for use as a medicament.

117. The compound or pharmaceutically acceptable salt thereof of any one of claims 1-65 or the pharmaceutical composition of claim 68 for use as a medicament in a method of treating lysosomal storage disease.

118. The compound or pharmaceutically acceptable salt thereof for use or the pharmaceutical composition for use of claim 117, wherein the lysosomal storage disease is Krabbe disease or Metachromatic Leukodystrophy.

119. The compound or pharmaceutically acceptable salt thereof of any one of claims 72-111 or the pharmaceutical composition of claim 112 for use as a medicament.

120. The compound or pharmaceutically acceptable salt thereof of any one of claims 72-111 or the pharmaceutical composition of claim 112, for use in a method of treating a lysosomal storage disease.

121. The compound or pharmaceutically acceptable salt thereof for use or the pharmaceutical composition for use of claim 120, wherein the lysosomal storage disease is Krabbe disease or Metachromatic Leukodystrophy.
